(19) United States
(12) Patent Application Publication
(10) Pub. No.: US 2004/0081958 A1
(43) Pub. Date: Apr. 29, 2004

(54) Identification and use of molecular markers indicating cellular reprogramming

(76) Inventors: Ken Eilertsen, Waterlo, WI (US); Martha Pfister-Genskow, Madison, WI (US); Lynette Childs, Madison, WI (US); Todd Forsythe, Ridgeway, WI (US); Michael D. Bishop, Rio, WI (US)

Correspondence Address: FOLEY & LARDNER, 402 WEST BROADWAY, 23RD FLOOR, SAN DIEGO, CA, 92101

(21) Appl. No.: 09/876,143

(22) Filed: Jun. 6, 2001

Related U.S. Application Data
(60) Provisional application No. 60/209,874, filed on Jun. 7, 2000.

(51) Int. Cl. C12Q 1/68; G06F 19/00; G01N 33/48; G01N 33/50; C07H 21/04

(52) U.S. Cl. 435/6; 702/20; 536/23.1

(57) ABSTRACT

The present invention is directed in part to methods and materials for identifying and evaluating the molecular events associated with cellular reprogramming. More particularly, the invention identifies one or more “expression events” occurring within cells, tissues, embryos, and/or animals that signal developmental competence or lineage-specific development. These expression events can be used to efficiently screen and select cells, tissues, embryos, and/or animals that are competent to undergo reprogramming from amongst a background of incompetent cells, tissues, embryos, and/or animals. Moreover, methods and molecules able to induce such expression events can be identified and used to induce competence in otherwise incompetent cells, tissues, embryos, and/or animals.

This application is related to, and claims priority from, provisional U.S. Patent Application No. 60/209,874, filed on Jun. 7, 2000, which is hereby incorporated by reference in its entirety, including all tables, figures, and claims.

### INTRODUCTION

The present invention relates in part to identifying and evaluating the molecular events associated with nuclear and cellular reprogramming. More particularly, the invention identifies one or more “expression events” occurring within cells, tissues, embryos, and/or animals that signal developmental competence or lineage-specific development.

### BACKGROUND OF THE INVENTION

The following description of the background of the invention is provided simply as an aid in understanding the invention and is not admitted to describe or constitute prior art to the invention.

Researchers have been developing methods for cloning animals over the past two decades. Some reported methods include the steps of (1) isolating a cell, most often an embryonic cell; (2) inserting that cell or a nucleus isolated from the cell into an enucleated oocyte (e.g., the nucleus of the oocyte was previously extracted), and (3) allowing an embryo to develop from the nuclear transfer oocyte in vivo. These methods, while useful, are severly limited due to poor efficiencies, as measured by the birth of live animals. In bovines, for example, embryos generated by in vitro fertilization techniques result in live births at a 50% or greater efficiency; artificial insemination techniques efficiencies are 90% or better. In contrast, live birth/nuclear transfer efficiencies are about 1% or less. Current methods to assess embryo viability and developmental competence rely on subjective measurements of embryo quality. See, e.g., Overström, 1996, Theriogenology 45: 3-16. In the context of nuclear transfer, these methods have proven to be of limited usefulness.

When a nuclear donor cell is inserted into a recipient oocyte, the oocyte environment alters the inserted nucleus in a process referred to as “cellular reprogramming.” This reprogramming can result in a developmentally competent nuclear transfer embryo; that is, an embryo able to result in a live birth. The underlying molecular mechanisms of cellular reprogramming remain poorly understood. Researchers have noted that DNA methylation patterns can be altered in the transition to developmental competence (see, e.g., Surani et al., 1990, Phil. Trans. R. Soc. Lond. B 326: 313-327; Monk, 1990, Phil. Trans. R. Soc. Lond. B 326: 299-312; Surani, 1999, Seminars in Cell and Dev. Biol. 10: 273-277); and that certain uridylic acid-rich nuclear RNA molecules and histone subtypes change as cells transition from developmental competence to a more differentiated state (see, e.g., Ray et al., 1997, Mol. and Cell. Biochem. 177: 79-88; Clarke et al., 1998, Dev. Genet. 22: 17-30).

Researchers have also described various gene products that may be related to pluripotence (i.e., the ability of a cell to differentiate into multiple cell lineages) and/or totipotence (i.e., the ability of a cell to differentiate into all the cells of an animal). Some possible examples are the oct-3 and oct-4 genes in mice (see, e.g., Rosner et al., 1990, Nature 345: 686-92; Shimazaki et al., 1993, EMBO J. 12: 4489-4498; Saijoh et al., 1996, Genes to Cells 1: 239-252; Wang and Schultz, 1996, Biochem. Cell Biol. 74: 579-584; Yeom et al., 1996, Development 122: 881-894; Brehm et al., 1997, Mol. and Cell. Biol. 17: 154-162; Brehm et al., Acta Pathol. Microbiol. et Immunol. Scand. 106: 114-126; Pesce et al., 1998, BioEssays 20: 722-732; and Pesce, 1999, Cells Tissues Organs 165: 144-152); and various mouse homeobox genes (see, e.g., Webb et al., 1993, Genomics 18: 464-466; and Chapman et al., 1997, Genomics 46: 223-33).

Moreover, researchers have also attempted to identify gene products that may be related to the ability of a pluripotent cell to differentiate into specific cell lineages, and to isolate specific stem cell populations. See, e.g., Bain et al., 1992, Soc. Neurosci. Abst. 18: 612 (abstract no. 265.13); Bain et al., 1993, Mol. Brain Res. 17: 23-30; Lelias et al., 1993, Proc. Natl. Acad. Sci. USA 90: 1479-1483; Urven et al., 1993, Biol. Reprod. 48: 564-574; U.S. Pat. No. 5,639,618, issued on Jun. 17, 1997 to Gay; Hendrikx et al., 1997, Exper. Hematol. 25: 878 (abstract no. 522); Walther and Bader, 1999, Mol. Brain Res. 68: 55-63; and U.S. Pat. No. 5,874,301, issued on Feb. 23, 1999 to Keller et al.

Additionally, researchers have developed a trap vector approach to identify potential developmentally related or lineage related genes. See, e.g., von Melchner et al., 1992, Genes and Dev. 6: 919-927; Reddy et al., 1992, Proc. Natl. Acad. Sci. USA 89: 6721-6725; Bruyns et al., 1994, Br. J. Haematol. 87 (Suppl. 1): 92 (abstract no. 362); Baker et al., 1997, Dev. Biol. 185: 201-214; Muth et al., 1998, Dev. Dynamics 212: 277-283; U.S. Pat. No. 5,922,601, issued on Jul. 13, 1999 to Baetscher et al.; and U.S. Pat. No. 5,928,888, issued on Jul. 27, 1999 to Whitney.

### SUMMARY OF THE INVENTION

The present invention concerns identifying and evaluating the molecular events associated with cellular reprogramming. More particularly, the invention identifies one or more “expression events” occurring within cells, tissues, embryos, and/or animals that signal developmental competence, developmental incompetence, lineage-specific development, viability, totipotency, or pluripotency. These expression events can then be used to efficiently screen and select cells, tissues, embryos, fetuses and/or animals that are competent to undergo reprogramming from amongst a background of incompetent cells, tissues, embryos, fetuses and/or animals. Moreover, methods and molecules able to induce such expression events can be identified and used to induce competence in otherwise incompetent cells, tissues, embryos, fetuses and/or animals.

The materials and methods described herein can be used to increase the efficiencies of cloning by nuclear transfer procedures from a success rate of less than 1% (measured by comparing the number of nuclear transfers required to produce a single live birth) to as much as 50% or more. Among the benefits provided are the ability to optimize culture conditions for competent donor cells and embryos, to optimize oocyte, donor cell, and embryo handling procedures, and to identify those donor cells, embryos and fetuses most likely to result in a live birth.

Furthermore, the materials and methods described herein can be used to increase the efficiencies of identifying cell populations for use in cell-based therapeutics and tissue regeneration. Among the benefits provided are the ability to optimize culture conditions for inducing stem cell populations to differentiate along a specific selected cell lineage, and to identify those stem cell populations most likely to provide a desired therapeutic benefit.

Thus, in a first aspect, the invention concerns the identification and use of one or more expressed sequence tags, the expression of which can be used to identify a cell, embryo, or fetus as being developmentally competent or developmentally incompetent.

In a first embodiment, cells can be identified as being developmentally competent based on the expression of an expressed sequence tag (or its complementary sequence) known to be present and/or expressed in a cell line that has been demonstrated to be developmentally competent, but that is present and/or expressed at a reduced or nondetectable level in a cell line that has been tested for, but has failed to demonstrate developmental competence. Similarly, cells can be identified as being developmentally incompetent based on the expression of an expressed sequence tag (or its complementary sequence) known to be present and/or expressed in a cell line that has been tested for, but has failed to demonstrate developmental competence, but that is present and/or expressed at a reduced or nondetectable level in a cell line that has been demonstrated to be developmentally competent.

The term “expressed sequence tag,” or “EST” as used herein refers to an isolated, enriched, or purified nucleic acid sequence representing a gene that is expressed in a tissue-specific or developmentally-specific manner by one or more cells. Such ESTs can be referred to as being “differentially expressed” in two cells or tissues. Preferably, an EST is obtained by sequencing one or more complementary DNA (“cDNA”) molecules prepared from messenger RNA (“mRNA”) strands present in a specific cell or tissue type. Methods for preparing cDNA molecules are well known to the skilled artisan. See, e.g., Sambrook, et al., 1989, Molecular Cloning: A Laboratory Manual, Second Edition, Cold Spring Harbor Press, Plainview, N.Y. In certain embodiments, an EST is obtained from sequences present in a tissue-specific or developmentally-specific cDNA library. An EST may represent the sequence of a full length gene or mRNA molecule, or may contain only a partial sequence. While an EST is preferably a sequence corresponding to an mRNA molecule itself (a “sense” sequence), in certain preferred embodiments an EST can be a sequence that is complementary to a nucleic acid molecule expressed in a tissue-specific or developmentally-specific manner (an “antisense” sequence). The term EST can refer to a sequence in any digital or alphanumeric form, such as a computer file, computer display, or printed table describing each sequence, or to the EST nucleic acid molecules themselves. An EST that is characteristic of a specific cell or tissue type may be referred to as a “marker” of that cell or tissue type.

While the present invention is described in terms of ESTs generated from a cDNA library, the skilled artisan will understand that nucleic acid sequences representing genes that are expressed in a tissue-specific or developmentally-specific manner by one or more cells can also be obtained from genomic DNA sequences. For example, Shoemaker et al., Nature 409: 922-927 describes microarray-based methods using exon arrays. Thus, the ESTs of the present invention may come from genomic sources, as well as from mRNA or cDNA sources.

Particularly preferred are one or more ESTs that are markers of developmental competence of cells, developmental incompetence of cells, developmental competence of embryos, developmental incompetence of embryos, lineage-specific development of cells, viability of cells, viability of embryos, viability of fetuses, totipotency of cells, pluripotency of cells, oocyte competence for nuclear transfer, oocyte incompetence for nuclear transfer, oocyte competence for in vitro fertilization, and oocyte incompetence for in vitro fertilization.

In preferred embodiments, an EST is at least about 9 nucleotides in length, at least about 10 nucleotides in length, at least about 11 nucleotides in length, at least about 12 nucleotides in length, at least about 13 nucleotides in length, at least about 14 nucleotides in length, at least about 15 nucleotides in length, at least about 16 nucleotides in length, at least about 17 nucleotides in length, at least about 18 nucleotides in length, at least about 19 nucleotides in length, at least about 20 nucleotides in length, at least about 25 nucleotides in length, at least about 30 nucleotides in length, at least about 35 nucleotides in length, at least about 40 nucleotides in length, at least about 45 nucleotides in length, at least about 50 nucleotides in length, at least about 55 nucleotides in length, at least about 60 nucleotides in length, at least about 65 nucleotides in length, at least about 70 nucleotides in length, at least about 75 nucleotides in length, at least about 80 nucleotides in length, at least about 90 nucleotides in length, at least about 100 nucleotides in length, at least about 125 nucleotides in length, at least about 150 nucleotides in length, at least about 175 nucleotides in length, at least about 200 nucleotides in length, at least about 300 nucleotides in length, at least about 400 nucleotides in length, at least about 500 nucleotides in length, at least about 1,000 nucleotides in length, at least about 5,000 nucleotides in length, at least about 10,000 nucleotides in length, at least about 50,000 nucleotides in length, and at least about 100,000 nucleotides in length.

The terms “complementary” and “complement” as used herein in reference to sequences refers to the ability of each of the various nucleotides to form a binding pair by hydrogen bonding with a specific complementary nucleotide. For example, the skilled artisan understands that guanine and cytosine are complementary nucleotides, as are adenine and thymine or uracil. A second sequence is complementary to a first sequence when substantially every nucleotide in the first sequence can be paired in register to a nucleotide in the second sequence. Two nucleic acid strands containing such complementary sequences can “hybridize,” or form a double stranded nucleic acid molecule. Nucleic acid hybridization techniques are well known in the art. See, e.g., Sambrook, et al., 1989, Molecular Cloning: A Laboratory Manual, Second Edition, Cold Spring Harbor Press, Plainview, N.Y.; U.S. Pat. No. 5,935,788, issued on Aug. 10, 1999 to Burmer et al., entitled “Subtractive Hybridization Techniques for Identifying Differentially Expressed and Commonly Expressed Nucleic Acid;” and U.S. Pat. No. 5,773,213, issued on Jun. 30, 1998 to Gullans et al., entitled “Method for Conducting Sequential Nucleic Acid Hybridization Steps,” each of which is incorporated in its entirety, including all tables, figures, and claims.

In preferred embodiments a second sequence is exactly complementary to a first sequence. In certain embodiments, however, a second sequence may contain one or more mismatched, additional, or deleted nucleotides and still be complementary to a first sequence, so long as the two strands contain sufficient complementary nucleotides for hybridization to occur. In preferred embodiments, a complementary sequence comprises 1% mismatched, additional, or deleted nucleotides, 2% mismatched, additional, or deleted nucleotides, 3% mismatched, additional, or deleted nucleotides, 4% mismatched, additional, or deleted nucleotides, 5% mismatched, additional, or deleted nucleotides, 6% mismatched, additional, or deleted nucleotides, 7% mismatched, additional, or deleted nucleotides, 8% mismatched, additional, or deleted nucleotides, 9% mismatched, additional, or deleted nucleotides, or 10% mismatched, additional, or deleted nucleotides. In particularly preferred embodiments, a sequence can be longer than its complementary sequence due to additional 5′ and/or 3′ nucleotides that do not overlap with the complementary region.

Isolated nucleic acids are unique in that they are not found in a pure or separated state in nature. Use of the term “isolated” indicates that a naturally occurring sequence has been removed from its normal cellular (i.e., chromosomal) environment. Thus, the sequence may be in a cell-free solution or placed in a different cellular environment. The term does not imply that the sequence is the only nucleic acid present, but that it is essentially free (about 90-95% pure at least) of non-nucleotide material naturally associated with it, and thus is distinguished from isolated chromosomes.

The term “enriched” in reference to nucleic acids means that the specific DNA or RNA molecule constitutes a significantly higher fraction (2- to 5-fold or more) of the total DNA or RNA present in the cells or solution of interest than in normal or diseased cells or in the cells from which the sequence was taken. This could be caused by a person by preferential reduction in the amount of other DNA or RNA present, or by a preferential increase in the amount of the specific DNA or RNA sequence, or by a combination of the two. However, it should be noted that enriched does not imply that there are no other DNA or RNA molecules present, just that the relative amount of the nucleic acid of interest has been significantly increased. The term “significant” is used to indicate that the level of increase is useful to the person making such an increase, and generally means an increase relative to other nucleic acids of about at least 2-fold, more preferably at least 5- to 10-fold or even more. The term also does not imply that there is no DNA or RNA from other sources. DNA from other sources may, for example, comprise DNA from a yeast or bacterial genome, or a cloning or expression vector.

It is also advantageous for some purposes that a nucleic acid molecule be in purified form. In this context, “purified” does not require absolute purity (such as a homogeneous preparation). Instead, it represents an indication that the molecule is relatively more pure than in its natural environment (compared to the natural level this level should be at least 2- to 5-fold greater, e.g., in terms of mg/mL). Individual clones isolated from a cDNA library may be purified to electrophoretic homogeneity. cDNA clones are not naturally occurring, but rather are preferably obtained via manipulation of a partially purified naturally occurring substance, typically messenger RNA (mRNA). The construction of a cDNA library from mRNA involves creating cDNAs by reverse transcription of mRNA. Pure individual cDNA clones can be isolated from the library by clonal selection. Thus, a process that includes the construction of a cDNA library from mRNA and isolation of distinct cDNA clones yields an approximate 10⁶-fold purification. Thus, purification of at least one order of magnitude, preferably two or three orders, and more preferably four or five orders of magnitude is expressly contemplated.

The term “expression” as used herein refers to the presence of an RNA molecule in a cell or tissue as a result of the transcription machinery of the cell or tissue. During transcription in eukaryotic cells, RNA molecules are synthesized from a complementary DNA template by one of three different RNA polymerase molecules. In most cases, the initial transcripted RNA molecule is not a functional RNA molecule, but is instead a precursor molecule that must be processed before it becomes a mature ribosomal, transfer, or messenger RNA molecule. Additionally, both primary transcripts and mature RNA molecules are subject to various degradation enzymes, and thus may be present as fragments of the original full length RNA molecule. The skilled artisan will therefore understand that expression can refer to the presence of the RNA molecule in any of these forms.

The term “developmentally competent” as used herein refers to a cell (or nucleus thereof), embryo, or fetus that is capable of developing into a live born animal. A developmentally competent cell can give rise to all of the cells of an animal when it is utilized as a source of nuclear donor material in a nuclear transfer procedure. In preferred embodiments, a “developmentally competent cell” has not yet been used in a nuclear transfer procedure, but is obtained from a cell line that has been demonstrated to produce cells that are capable of developing into a live born animal. Such a cell line is referred to as a “developmentally competent cell line.” A developmentally competent cell can be referred to as “totipotent.” A developmentally competent cell may be, but need not be, capable of passing its genetic characteristics through the germ line. In preferred embodiments, a developmentally competent cell line is so identified if 50%, 60%, 70%, 80%, or 90% of nuclear transfer embryos prepared using nuclear donors from that cell line are able to initiate pregnancy and reach 90 days of gestation in a maternal host. In other preferred embodiments, a developmentally competent cell line is so identified if 50%, 60%, 70%, 80%, or 90% of nuclear transfer embryos prepared using nuclear donors from that cell line are able to initiate pregnancy in a maternal host, and 50%, 60%, 70%, 80%, or 90% of those pregnancies result in a live birth.

The term “developmentally competent cell” and “developmentally competent cell line” may also refer to cells and cell lines expressing one or more nucleic acid sequences that are known to be present and/or expressed in a cell line that has been demonstrated to be developmentally competent, but that are present and/or expressed at a reduced or nondetectable level in a cell line that has been tested for, but has failed to demonstrate developmental competence. Such a nucleic acid can be referred to as a “marker” of a developmentally competent cell or cell line.

The term “developmentally incompetent” as used herein refers to a cell (or nucleus thereof), embryo, or fetus that is not capable of developing into a live born animal. In particularly preferred embodiments, a developmentally incompetent cell can give rise to all of the cells of an embryo or fetus when it is utilized as a source of nuclear donor material in a nuclear transfer procedure, but is incapable of giving rise to a live born animal. Thus, a developmentally incompetent cell may be “pluripotent,” but is not “totipotent.” In preferred embodiments, a “developmentally incompetent cell” is obtained from a cell line that has been tested for the ability to develop into a live born animal under conditions successfully used with developmentally competent cells, but has failed to demonstrate developmental competence. Such a cell line is referred to as a “developmentally incompetent cell line.” In preferred embodiments, a developmentally competent cell line is so identified if less than 50%, less than 40%, less than 30%, less than 20%, or less than 10% of nuclear transfer embryos prepared using nuclear donors from that cell line are able to initiate pregnancy and reach 90 days of gestation in a maternal host.

The term “developmentally incompetent cell” and “developmentally incompetent cell line” may also refer to cells and cell lines expressing one or more nucleic acid sequences that are known to be present and/or expressed in a cell line that has been tested for, but has failed to demonstrate developmental competence, but that are present and/or expressed at a reduced or nondetectable level in a cell line that has been demonstrated to be developmentally competent. Such a nucleic acid can be referred to as a “marker” of a developmentally incompetent cell or cell line.

In preferred embodiments, developmentally competent and incompetent cells include, but are not limited to, cells isolated from an embryo arising from the union of two gametes in vitro or in vivo; embryonic stem cells (ES cells) arising from cultured embryonic cells (e.g., pre-blastocyst cells and inner cell mass cells); inner cell mass cells isolated from of embryos; pre-blastocyst cells; fetal cells; primordial germ cells; germ cells (e.g., embryonic germ cells); somatic cells isolated from an animal; cumulus cells; amniotic cells; fetal fibroblast cells; genital ridge cells; differentiated cells; lineage-specific cells; and totipotent cells.

The term “identifies” or “identifying” as used herein with respect to cells refers to the ability to distinguish between cells having two distinct characteristics. In preferred embodiments, a developmentally competent cell or cell line can be distinguished from a developmentally incompetent cell or cell line. In certain preferred embodiments, an EST or ESTs identify a cell or cell line as “developmentally competent” if the EST sequences are present and/or expressed in embryos produced by nuclear transfer using a developmentally competent nuclear donor cell, but are present and/or expressed at a reduced or nondetectable level in embryos produced by nuclear transfer using a developmentally incompetent nuclear donor cell. Similarly, an EST or ESTs identify a cell or cell line as “developmentally incompetent” if the sequences are present and/or expressed in embryos produced by nuclear transfer using a developmentally incompetent nuclear donor cell, but are present and/or expressed at a reduced or nondetectable level in embryos produced by nuclear transfer using a developmentally competent nuclear donor cell.

The term “detectable level” as used herein refers to the ability of a comparison method to detect a nucleic acid molecule. The skilled artisan will understand that different comparison methods will have different sensitivities. For example, an RNA molecule present in low abundance in a cell may be below the detectable level of a hybridization assay due to the hybridization conditions used. Moreover, detection of a protein product by immunological means may not detect RNA molecules present in even moderate abundance. But it is well within the skill level of the ordinarily skilled artisan to determine which comparison methods may be appropriately used in specific circumstances. For example, a developmentally regulated RNA molecule may be present in high abundance in one developmental stage, but present in moderate abundance in a second developmental stage. The two developmental stages may be differentiated by a comparison method in which moderate abundance is below the detectable level, but high abundance detectable.

The term “totipotent” as used herein refers to a cell that gives rise to a live born animal. The term “totipotent” can also refer to a cell that gives rise to all of the cells in a particular animal. A totipotent cell can give rise to all of the cells of an animal when it is utilized in a procedure for developing an embryo from one or more nuclear transfer steps. Totipotent cells may also be used to generate incomplete animals such as those useful for organ harvesting, e.g., having genetic modifications to eliminate growth of an organ or appendage by manipulation of a homeotic gene. A totipotent cell may be, but need not be, capable of passing its genetic characteristics through the germ line.

The term “totipotent” as used herein is to be distinguished from the term “pluripotent.” The latter term refers to a cell capable of differentiating into a number of different cell types, but that cannot give rise to all of the cells in a live born animal. The term “totipotent” as used herein is also to be distinguished from the term “chimer” or “chimera.” The latter term refers to a developing cell mass, such as an embryo, fetus, or animal, that comprises a sub-group of cells harboring nuclear DNA with a significantly different nucleotide base sequence than the nuclear DNA of other cells in that cell mass.

The term “live born” as used herein preferably refers to an animal that exists ex utero. A “live born” animal may be an animal that is alive for at least one second from the time it exits the maternal host. A “live born” animal may not require the circulatory system of an in utero environment for survival. A “live born” animal may be an ambulatory animal. Such animals can include pre- and post-pubertal animals. As discussed previously, a live born animal may lack a portion of what exists in a physiologically normal animal of its kind.

In preferred embodiments, developmentally competent cells and developmentally incompetent cells are cultured; are cultured as cell lines; and are cultured as permanent cell lines.

The term “cultured” as used herein in reference to cells can refer to one or more cells that are undergoing cell division or not undergoing cell division in an in vitro environment. An in vitro environment can be any medium known in the art that is suitable for maintaining cells in vitro, such as suitable liquid media or agar, for example. Specific examples of suitable in vitro environments for cell cultures are described in Culture of Animal Cells: a manual of basic techniques (3^{rd} edition), 1994, R. I. Freshney (ed.), Wiley-Liss, Inc.; Cells: a laboratory manual (vol. 1), 1998, D. L. Spector, R. D. Goldman, L. A. Leinwand (eds.), Cold Spring Harbor Laboratory Press; and Animal Cells: culture and media, 1994, D. C. Darling, S. J. MorganJohn Wiley and Sons, Ltd., each of which is incorporated herein by reference in its entirety including all figures, tables, and drawings. Cells may be cultured in suspension and/or in monolayers with one or more substantially similar cells. Cells may be cultured in suspension and/or in monolayers with a heterogeneous population of cells. The term “heterogeneous” as utilized in the previous sentence can relate to any cell characteristics, such as cell type and cell cycle stage, for example. Cells may be cultured in suspension, cultured as monolayers attached to a solid support, and/or cultured on a layer of feeder cells, for example. Furthermore, cells may be successfully cultured by plating the cells in conditions where they lack cell to cell contact. Cells cultured as monolayers may be grown to confluence, where such cells will cease actively dividing due to contact inhibition. Preferably, cultured cells undergo cell division and are cultured for at least 5 days, more preferably for at least 10 days or 20 days, and most preferably for at least 30 days. Preferably, a significant number of cultured cells do not terminate while in culture. The terms “terminate” and “significant number are defined” hereafter. Nearly any type of cell can be placed in cell culture conditions. Cultured cells can be utilized to establish a cell line.

The term “cell line” as used herein refers to cultured cells that can be passaged at least one time without terminating. The invention relates to cell lines that can be passaged at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 80, 100, and 200 or more times. Cell passaging is defined hereafter. Examples of cell lines include, but are not limited to, cell lines derived from cells isolated from an embryo arising from the union of two gametes in vitro or in vivo; embryonic stem cell lines (ES cells) arising from cultured embryonic cells (e.g., pre-blastocyst cells and inner cell mass cells); cell lines arising from inner cell mass cells isolated from of embryos; cell lines obtained from pre-blastocyst cells; cell lines obtained from fetal cells; cell lines obtained from primordial germ cells; cell lines obtained from germ cells (e.g., embryonic germ cells); cell lines obtained from somatic cells isolated from an animal; cell lines obtained from cumulus cells; cell lines obtained from amniotic cells; cell lines obtained from fetal fibroblast cells; cell lines obtained from genital ridge cells; cell lines obtained from differentiated cells; lineage-specific cell lines; and cell lines obtained from totipotent cells.

In preferred embodiments (1) a cell or a cell line of the present invention is a mammalian cell or cell line; (2) a mammalian cell or cell line is selected from the group consisting of canid cells or cell lines, feuid cells or cell lines, murid cells or cell lines, leporid cells or cell lines, ursid cells or cell lines, mustelid cells or cell lines, and human and non-human primate cells or cell lines; (3) a mammalian cells or cell lines is an ungulate cells or cell lines; and (4) an ungulate cells or cell lines is selected from the group consisting of suid cells or cell lines, ovid cells or cell lines, equid cells or cell lines, bovid cells or cell lines, caprid cells or cell lines, and cervid cells or cell lines.

The term “mammalian” as used herein refers to any animal of the class Mammalia. Preferably, a mammalian cell or cell line is a placental, a monotreme and a marsupial. Most preferably, a mammalian cell or cell line is a bovine, a porcine, and a human and non-human primate. A mammalian cell or cell line can be isolated from any source of mammalian cells including, but not limited to, a mammalian embryo, a mammalian fetus, and a mammalian animal.

The term “canid” as used herein refers to any animal of the family Canidae. Preferably, a canid cell or cell line is isolated from a wolf, a jackal, a fox, and a domestic dog.

The term “felid” as used herein refers to any animal of the family Felidae. Preferably, a felid cell or cell line is isolated from a lion, a tiger, a leopard, a cheetah, a cougar, and a domestic cat.

The term “murid” as used herein refers to any animal of the family Muridae. Preferably, a murid cell or cell line is isolated from a mouse and a rat.

The term “leporid” as used herein refers to any animal of the family Leporidae. Preferably, a leporid cell or cell line is isolated from a rabbit.

The term “ursid” as used herein refers to any animal of the family Ursidae. Preferably, a ursid cell or cell line is isolated from a bear.

The term “mustelid” as used herein refers to any animal of the family Musteldae. Preferably, a mustelid cell or cell line is isolated from a weasel, a ferret, an otter, a mink, and a skunk.

The term “primate” as used herein refers to any animal of the Primate order. Preferably, a primate cell or cell line is isolated from an ape, a monkey, a chimpanzee, and a lemur.

The term “ungulate” as used herein refers to any animal of the polyphyletic group formerly known as the taxon Ungulata. Preferably, an ungulate cell or cell line is isolated from a camel, a hippopotamus, a horse, a tapir, and an elephant. Most preferably, an ungulate cell or cell line is isolated from a sheep, a cow, a goat, and a pig.

The term “ovid” as used herein refers to any animal of the family Ovidae. Preferably, an ovid cell or cell line is isolated from a sheep.

The term “suid” as used herein refers to any animal of the family Suidae. Preferably, a suid cell or cell line is isolated from a pig or a boar.

The term “equid” as used herein refers to any animal of the family Equidae. Preferably, an equid cell or cell line is isolated from a zebra or an ass. Most preferably, an equid cell or cell line is isolated from a horse.

The term “bovid” as used herein refers to any animal of the family Bovidae. Preferably, an bovid cell or cell line is isolated from an antelope, an oxen, a cow, a bison, and a goat.

The term “caprid” as used herein refers to any animal of the family Caprinae. Preferably, an caprid cell or cell line is isolated from a goat.

The term “cervid” as used herein refers to any animal of the family Cervidae. Preferably, an cervid cell or cell line is isolated from a deer.

The term “terminating” and “termiinate” as used herein with regard to cultured cells may refer to cells that undergo cell death, which can be measured using multiple techniques known to those skilled in the art (e.g., CytoTox96® Cytotoxicity Assay, Promega, Inc. catalog no. G1780; Celltiter96® Aqueous Cell Proliferation Assay Kit, Promega, Inc. catalog no. G3580; and Trypan Blue solution for cytotoxicity assays, Sigma catalog no. T6146). Termination may also be a result of apoptosis, which can be measured using multiple techniques known to persons skilled in the art (e.g., Dead End™ Apoptosis Detection Kit, Promega, Inc. catalog no. G7130). Terminated cells may be identified as those that have undergone cell death and/or apoptosis and have released from a solid surface in culture. In addition, terminated cells may lack intact membranes which can be identified by procedures described above. Also, terminated cells may exhibit decreased metabolic activity, which may be caused in part by decreased enzymatic activity that can be identified by calcein AM, for example. Furthermore, termination can be refer to cell cultures where a significant number of cultured cells terminate. The term “significant number” in the preceding sentence can refer to about 80% of the cells in culture, preferably about 90% of the cells in culture, more preferably about 100% of the cells in culture, and most preferably 100% of the cells in culture.

The term “suspension” as used herein refers to cell culture conditions in which cells are not attached to a solid support. Cells proliferating in suspension can be stirred while proliferating using apparatus well known to those skilled in the art.

The term “monolayer” as used herein refers to cells that are attached to a solid support while proliferating in suitable culture conditions. A small portion of cells proliferating in a monolayer under suitable growth conditions may be attached to cells in the monolayer but not to the solid support. Preferably less than 15% of these cells are not attached to the solid support, more preferably less than 10% of these cells are not attached to the solid support, and most preferably less than 5% of these cells are not attached to the solid support.

The term “plated” or “plating” as used herein in reference to cells can refer to establishing cell cultures in vitro. For example, cells can be diluted in cell culture media and then added to a cell culture plate, dish, or flask. Cell culture plates are commonly known to a person of ordinary skill in the art. Cells may be plated at a variety of concentrations and/or cell densities.

The term “cell plating” can also extend to the term “cell passaging.” Cells of the invention can be passaged using cell culture techniques well known to those skilled in the art. The term “cell passaging” can refer to a technique that involves the steps of (1) releasing cells from a solid support or substrate and disassociation of these cells, and (2) diluting the cells in media suitable for further cell proliferation. Cell passaging may also refer to removing a portion of liquid medium containing cultured cells and adding liquid medium to the original culture vessel to dilute the cells and allow further cell proliferation. In addition, cells may also be added to a new culture vessel which has been supplemented with medium suitable for further cell proliferation.

The term “proliferation” as used herein in reference to cells can refer to a group of cells that can increase in number over a period of time.

The term “confluence” as used herein refers to a group of cells where a large percentage of cells are physically contacted with at least one other cell in that group. Confluence may also be defined as a group of cells that grow to a maximum cell density in the conditions provided. For example, if a group of cells can proliferate in a monolayer and they are placed in a culture vessel in a suitable growth medium, they are confluent when the monolayer has spread across a significant surface area of the culture vessel. The surface area covered by the cells preferably represents about 50% of the total surface area, more preferably represents about 70% of the total surface area, and most preferably represents about 90% of the total surface area.

In further embodiments, expressed sequence tags can be grouped in numbers of 2 or more, and up to numbers of 10,000 or more, to provide a gene expression database. The expression of one or more expressed sequence tags in the database can be used to identify cells, embryos, or fetuses as being developmentally competent or developmentally incompetent.

Preferably, a gene expression database comprises two or more expressed sequence tags (or their complementary sequences) known to be present and/or expressed in a cell line that has been demonstrated to be developmentally competent, but that are present and/or expressed at a reduced or nondetectable level in a cell line that has been tested for, but has failed to demonstrate developmental competence. As discussed above, such ESTs can be referred to as being “differentially expressed.” Cells, embryos, and fetuses can be identified as developmentally competent based on the presence of at least one of the ESTs in such a gene expression database. In particularly preferred embodiments, a cell, embryo or fetus is identified as developmentally competent based on the presence of at least about 75% of the ESTs in such a gene expression database; at least about 90% of the ESTs in such a gene expression database; at least about 95% of the ESTs in such a gene expression database; and about 100% of the ESTs in such a gene expression database.

Likewise, a gene expression database preferably comprises two or more expressed sequence tags (or their complementary sequences) known to be present and/or expressed in a cell line that has been tested for, but has failed to demonstrate developmental competence, but that are present and/or expressed at a reduced or nondetectable level in a cell line that has been demonstrated to be developmentally competent. Cells, embryos, and fetuses can be identified as developmentally incompetent based on the presence of at least one of the former ESTs, and the absence of the latter ESTs. In particularly preferred embodiments, a cell, embryo or fetus is identified as developmentally incompetent based on the presence of at least about 75% of the ESTs in such a gene expression database; at least about 90% of the ESTs in such a gene expression database; at least about 95% of the ESTs in such a gene expression database; and about 100% of the ESTs in such a gene expression database.

Most preferably, a gene expression database comprises at least one EST (or its complementary sequence) known to be present and/or expressed in a cell line that has been demonstrated to be developmentally competent, but that is present and/or expressed at a reduced or nondetectable level in a cell line that has been tested for, but has failed to demonstrate developmental competence; and at least one EST (or its complementary sequence) known to be present and/or expressed in a cell line that has been tested for, but has failed to demonstrate developmental competence, but that is present and/or expressed at a reduced or nondetectable level in a cell line that has been demonstrated to be developmentally competent. In such embodiments, cells, embryos, and fetuses can be identified as developmentally competent based on the presence of at least one of the former ESTs, and the absence of the latter ESTs. Likewise, cells can be identified as developmentally incompetent based on the presence of at least one of the latter ESTs, and the absence of the former ESTs.

The term “gene expression database” as used herein refers to any set of two or more different ESTs. In certain preferred embodiments, a gene expression database can be a representation of two or more EST sequences in any digital or alphanumeric form, such as a computer file, computer display, or printed table describing each sequence. In other preferred embodiments, a gene expression database can be any format containing the EST nucleic acid molecules themselves. For example, a solution or a solid phase comprising two or more different ESTs can be a gene expression database as that term is used in the instant invention. In preferred embodiments, a gene expression database can contain at least about 2, 3, 4, 5, 10, 15, 20, 25, 30, 40, 50, 75, 100, 200, 500, 1000, 2000, 5000, 10000, 20000, 25000, 30000, 40000, 50000, or 100,000 different ESTs.

Particularly preferred are gene expression databases that contain one or more markers of developmental competence of cells, developmental incompetence of cells, developmental competence of embryo s, developmental incompetence of embryos, lineage-specific development of cells, viability of cells, viability of embryos, totipotency of cells, pluripotency of cells, oocyte competence for nuclear transfer, oocyte incompetence for nuclear transfer, oocyte competence for in vitro fertilization, and oocyte incompetence for in vitro fertilization.

The term “plurality” as used herein refers to 2 or more. In preferred embodiments, a plurality can be 3, 4, 5, 10, 15, 20, 25, 30, 40, 50, 75, 100, 200, 500, 1000, 2000, 5000, 10000, 20000, 25000, 30000, 40000, 50000, or 100000 or more.

In yet other embodiments, the invention relates to methods for identifying one or more expressed sequence tags, the expression of which can be used to identify cells, embryos, or fetuses as being developmentally competent or developmentally incompetent.

In preferred embodiments, one or more ESTs are identified by comparing one or more first nucleic acid molecules obtained from one or more embryos produced by nuclear transfer using a developmentally competent nuclear donor cell to one or more second nucleic acid molecules obtained from one or more embryos produced by nuclear transfer using a developmentally incompetent nuclear donor cell. ESTs that signal developmental competence are identified as one or more nucleic acid molecules that are present in the population of first nucleic acid molecules, but that are not present at a detectable level in the population of second nucleic acid molecules. Likewise, one or more nucleic acid molecules that are present in the population of second nucleic acid molecules, but that are not present at a detectable level in the population of first nucleic acid molecules are identified as ESTs that signal developmental incompetence.

In particularly preferred embodiments, an EST that signals developmental competence is a nucleic acid molecule (1) present in at least about 75% of embryos produced by nuclear transfer using a developmentally competent nuclear donor cell that are tested for the presence of the EST, but not present at a detectable level in at least about 75% of tested embryos produced by nuclear transfer using a developmentally incompetent nuclear donor cell; (2) present in at least about 90% of embryos produced by nuclear transfer using a developmentally competent nuclear donor cell that are tested for the presence of the EST, but not present at a detectable level in at least about 90% of tested embryos produced by nuclear transfer using a developmentally incompetent nuclear donor cell; (3) present in at least about 95% of embryos produced by nuclear transfer using a developmentally competent nuclear donor cell that are tested for the presence of the EST, but not present at a detectable level in at least about 95% of tested embryos produced by nuclear transfer using a developmentally incompetent nuclear donor cell; and (4) present in at least about 100% of embryos produced by nuclear transfer using a developmentally competent nuclear donor cell that are tested for the presence of the EST, but not present at a detectable level in at least about 100% of tested embryos produced by nuclear transfer using a developmentally incompetent nuclear donor cell.

In other particularly preferred embodiments, an EST that signals developmental incompetence is a nucleic acid molecule (1) present in at least about 75% of embryos produced by nuclear transfer using a developmentally incompetent nuclear donor cell that are tested for the presence of the EST, but not present at a detectable level in at least about 75% of tested embryos produced by nuclear transfer using a developmentally competent nuclear donor cell; (2) present in at least about 90% of embryos produced by nuclear transfer using a developmentally incompetent nuclear donor cell that are tested for the presence of the EST, but not present at a detectable level in at least about 90% of tested embryos produced by nuclear transfer using a developmentally competent nuclear donor cell; (3) present in at least about 95% of embryos produced by nuclear transfer using a developmentally incompetent nuclear donor cell that are tested for the presence of the EST, but not present at a detectable level in at least about 95% of tested embryos produced by nuclear transfer using a developmentally competent nuclear donor cell; and (4) present in at least about 100% of embryos produced by nuclear transfer using a developmentally incompetent nuclear donor cell that are tested for the presence of the EST, but not present at a detectable level in at least about 100% of tested embryos produced by nuclear transfer using a developmentally competent nuclear donor cell.

The term “comparing” as used herein in reference to nucleic acid molecules refers to the process of determining the homology or identity of a first nucleic acid sequence to a second nucleic acid sequence. Methods for comparing two nucleic acid sequences are well known to the skilled artisan. In preferred embodiments, such methods can comprise comparing the two sequences in any digital or alphanumeric form, such as a computer file, computer display, or printed table describing each sequence. In this case, comparisons can be made by eye, that is, by a direct comparison by the skilled artisan, or can rely on various computer programs known in the art. See, e.g., Altschul, et al. (1997) Nucleic Acids Res. 25:3389-3402 (Gapped BLAST or PSI-BLAST), Altschul, et al. (1990) J. Mol. Biol. 215:403-410 (BLAST), and Smith, et al. (1981) J. Mol. Biol. 147:195-197 (Smith-Waterman). In other preferred embodiments, comparison methods can comprise comparing two nucleic acid molecules themselves, for example by hybridization methods such as southern blotting, northern blotting, in situ hybridization, dot or slot blotting, arrayed nucleic acids (including nucleic acid macroarrays and microarrays, particularly DNA macroarrays and microarrays), and phage display. See generally, Sambrook, et al., 1989, Molecular Cloning: A Laboratory Manual, Second Edition, Cold Spring Harbor Press, Plainview, N.Y.; U.S. Pat. No. 6,004,755, issued on Dec. 21, 1999 to B. Wang, each of which is hereby incorporated in its entirety, including all drawings, claims, and tables. In such methods, typically a nucleic acid molecule that is complementary to a first sequence is compared to a second sequence. In particularly preferred embodiments, two nucleic acid molecules can be compared indirectly by comparing each nucleic acid to a reference nucleic acid library, preferably obtained from the same species as the source of the nucleic acid molecules. The term “nucleic acid library” is defined herein. The foregoing examples are not intended to be exclusive, and other methods for comparing two nucleic acid sequences known in the art are within the scope of the instant invention.

The term “comparing” may also refer to determining the homology or identity of one nucleic acid sequence to another by determining the homology or identity molecules produced from the nucleic acid sequences. For example, mRNA can be used by cells or by in vitro translation systems to produce proteins or peptides. These peptides or proteins can be compared by various immunological methods such as immunoblotting, competitive or noncompetitive immunoassay, and immunoprecipitation, and by various nonimmunological methods such as analytical centrifugation, amino acid analysis, sequencing, 1- and 2-dimensional electrophoresis (including both native and denaturing conditions such as SDS-PAGE), chromatography, peptide mapping, nuclear magnetic resonance, electron crystallography, and X-ray crystallography. See generally, Deutscher, ed., 1990, Methods in Enzymology, Volume 182, Academic Press, San Diego, Calif. Such methods can be referred to by the skilled artisan as “proteomics” or “functional proteomics” techniques.

In preferred embodiments, a nucleic acid sequence or molecule is at least about 9 nucleotides in length, at least about 10 nucleotides in length, at least about 11 nucleotides in length, at least about 12 nucleotides in length, at least about 13 nucleotides in length, at least about 14 nucleotides in length, at least about 15 nucleotides in length, at least about 16 nucleotides in length, at least about 17 nucleotides in length, at least about 18 nucleotides in length, at least about 19 nucleotides in length, at least about 20 nucleotides in length, at least about 25 nucleotides in length, at least about 30 nucleotides in length, at least about 35 nucleotides in length, at least about 40 nucleotides in length, at least about 45 nucleotides in length, at least about 50 nucleotides in length, at least about 55 nucleotides in length, at least about 60 nucleotides in length, at least about 65 nucleotides in length, at least about 70 nucleotides in length, at least about 75 nucleotides in length, at least about 80 nucleotides in length, at least about 90 nucleotides in length, at least about 100 nucleotides in length, at least about 125 nucleotides in length, at least about 150 nucleotides in length, at least about 175 nucleotides in length, at least about 200 nucleotides in length, at least about 300 nucleotides in length, at least about 400 nucleotides in length, at least about 500 nucleotides in length, at least about 750 nucleotides in length, at least about 1000 nucleotides in length, at least about 1250 nucleotides in length, at least about 1500 nucleotides in length, at least about 2000 nucleotides in length, and at least about 3000 nucleotides in length.

The term “homology” as used herein in reference to nucleic acid molecules refers to the amount of sequence similarity between a first and a second nucleic acid molecule. Two molecules displaying sufficient homology are said to be “homologous” to one another. The skilled artisan will understand that a second sequence may contain one or more mismatched, additional, or deleted nucleotides and still be homologous to a first sequence. In preferred embodiments, a homologous sequence comprises 1% mismatched, additional, or deleted nucleotides, 2% mismatched, additional, or deleted nucleotides, 3% mismatched, additional, or deleted nucleotides, 4% mismatched, additional, or deleted nucleotides, 5% mismatched, additional, or deleted nucleotides, 6% mismatched, additional, or deleted nucleotides, 7% mismatched, additional, or deleted nucleotides, 8% mismatched, additional, or deleted nucleotides, 9% mismatched, additional, or deleted nucleotides, or 10% mismatched, additional, or deleted nucleotides. A sequence displaying no mismatched, additional, or deleted nucleotides is said to be “identical” to a first sequence. In particularly preferred embodiments, a sequence can be longer than a homologous or identical sequence due to additional 5′ and/or 3′ nucleotides that do not overlap with the homologous or identical region.

In certain embodiments, two molecules are referred to as homologous if they contain sufficient sequence identity that a third nucleic acid molecule used as a probe is capable of hybridizing to both molecules. In particularly preferred embodiments, the probe molecule is complementary to one of the two homologous molecules. The skilled artisan will understand that the amount of homology required between the two molecules such that a probe will bind to both can be variable, depending on the stringency of the hybridization conditions employed.

Homology of two nucleic acid molecules may also be determined from assessing the amount of sequence similarity between a first and a second molecule produced from the nucleic acid sequences. For example, peptides or proteins can be compared by the various methods described above, and homologous nucleic acids identified based on similar or identical peptide maps, amino acid sequences, antibody bindings, etc.

The term “identifying” as used herein with respect to nucleic acid molecules refers to selecting one or more molecules exhibiting identity or homology to a target nucleic acid sequence of interest. In preferred embodiments, identifying can refer to selecting sequences representing one or more nucleic acid molecules in any digital or alphanumeric form, such as a computer file, computer display, or printed table describing each sequence. In other preferred embodiments, identifying can comprise selecting one or more nucleic acid molecules themselves.

The terms “nuclear transfer” and “nuclear transfer procedure” as used herein refers to introducing a full complement of nuclear DNA from one cell to an enucleated cell. Nuclear transfer methods are well known to a person of ordinary skill in the art. See, U.S. Pat. No. 4,994,384 to Prather et al., entitled “Multiplying Bovine Embryos,” issued on Feb. 19, 1991; U.S. Pat. No. 5,057,420 to Massey, entitled “Bovine Nuclear Transplantation,” issued on Oct. 15, 1991; U.S. Pat. No. 5,994,619, issued on Nov. 30, 1999 to Stice et al.; U.K. Patents Nos. GB 2,318,578 GB 2,331,751, issued on Jan. 19, 2000 to Campbell et al. and Wilmut et al., respectively, entitled “Quiescent Cell Populations For Nuclear Transfer”; U.S. Pat. No. 6,011,197 to Strelchenko et al., entitled “Method of Cloning Bovines Using Reprogrammed Non-Embryonic Bovine Cells,” issued on Jan. 4, 2000; U.S. Pat. No. 6,107,543; Proc. Nat'l. Acad. Sci. USA 96: 14984-14989 (1999); Nature Genetics 22: 127-128 (1999); Cell & Dev. Diol 10: 253-258 (1999); Nature Biotechnology 17: 456-461 (1999); Science 289: 1188-1190 (2000); Nature Biotechnol. 18: 1055-1059 (2000); and Nature 407: 86-90 (2000); each of which is incorporated herein by reference in its entirety, including all figures, tables, and drawings.

In a nuclear transfer procedure, a nuclear donor cell, or the nucleus thereof, is introduced into a recipient cell. A recipient cell is preferably an oocyte and is preferably enucleated. However, the invention relates in part to nuclear transfer, where a nucleus of an oocyte is not physically extracted from the nucleus. It is possible to establish a nuclear transfer embryo where nuclear DNA from the donor cell is replicated during cellular divisions. See, e.g., Wagoner et al., 1996, “Functional enucleation of bovine oocytes: effects of centrifugation and ultraviolet light,” Theriogenology 46: 279-284. In addition, nuclear transfer may be accomplished by combining one nuclear donor and more than one enucleated oocyte. Also, nuclear transfer may be accomplished by combining one nuclear donor, one or more enucleated oocytes, and the cytoplasm of one or more enucleated oocytes. The resulting combination of a nuclear donor cell and a recipient cell can be referred to variously as a “nuclear transfer embryo,” a “hybrid cell,” or a “cybrid.”

Furthermore, a nuclear donor may arise from an animal of the same specie from which a nuclear recipient is isolated. Alternatively, a nuclear donor may arise from an animal of a different specie from which a nuclear recipient is isolated. For example, a differentiated cell isolated from an ear punch of a water buffalo may be utilized as a nuclear donor and an oocyte isolated from a bovine animal may be utilized as a nuclear acceptor. Thus, xenospecific nuclear transfer is contemplated by the instant invention.

The term “nuclear donor” as used herein refers to any cell having nuclear DNA that can be translocated into an oocyte. A nuclear donor may be a nucleus that has been isolated from a cell. Multiple techniques are available to a person of ordinary skill in the art for isolating a nucleus from a cell and then utilizing the nucleus as a nuclear donor. See, e.g., U.S. Pat. No. 4,664,097, which is hereby incorporated by reference in its entirety including all figures, tables and drawings. Any type of cell can serve as a nuclear donor. Examples of nuclear donor cells include, but are not limited to, cultured and non-cultured cells isolated from an embryo arising from the union of two gametes in vitro or in vivo; embryonic stem cells (ES cells) arising from cultured embryonic cells (e.g., pre-blastocyst cells and inner cell mass cells); cultured and non-cultured cells arising from inner cell mass cells isolated from of embryos; cultured and non-cultured pre-blastocyst cells; cultured and non-cultured fetal cells; cultured and non-cultured primordial germ cells; cultured and non-cultured germ cells (e.g., embryonic germ cells); cultured and non-cultured somatic cells isolated from an animal; cultured and non-cultured cumulus cells; cultured and non-cultured amniotic cells; cultured and non-cultured fetal fibroblast cells; cultured and non-cultured genital ridge cells; cultured and non-cultured differentiated cells; cultured and non-cultured cells in a synchronous population; cultured and non-cultured cells in an asynchronous population; cultured and non-cultured serum-starved cells; cultured and non-cultured permanent cells; and cultured and non-cultured totipotent cells. See, e.g., Piedrahita et al., 1998, Biol. Reprod. 58: 1321-1329; Shim et al., 1997, Biol. Reprod. 57: 1089-1095; Tsung et al., 1995, Shih Yen Sheng Wu Hsueh Pao 28: 173-189; and Wheeler, 1994, Reprod. Fertil. Dev. 6: 563-568, each of which is incorporated herein by reference in its entirety including all figures, drawings, and tables. In addition, a nuclear donor may be a cell that was previously frozen or cryopreserved.

In particularly preferred embodiments, a nuclear donor cell is a transgenic cell. The term “transgenic” as used herein in reference to cells refers to a cell whose genome has been altered using recombinant DNA techniques. In preferred embodiments, a transgenic cell comprises one or more exogenous DNA sequences in its genome. In other preferred embodiments, a transgenic cell comprises a genome in which one or more endogenous genes have been deleted, duplicated, activated, or modified. In particularly preferred embodiments, a transgenic cell comprises a genome having both one or more exogenous DNA sequences, and one or more endogenous genes that have been deleted, duplicated, activated, or modified.

The term “activation” refers to any materials and methods useful for stimulating a cell to divide before, during, and after a nuclear transfer step. Cybrids may require stimulation in order to divide after a nuclear transfer has occurred. The invention pertains to any activation materials and methods known to a person of ordinary skill in the art. Although electrical pulses are sometimes sufficient for stimulating activation of cybrids, other means are sometimes useful or necessary for proper activation of the cybrid. Chemical materials and methods useful for activating embryos are described below in other preferred embodiments of the invention.

Examples of non-electrical means for activation include agents such as ethanol; inositol trisphosphate (IP₃); Ca⁺⁺ ionophores (e.g., ionomycin) and protein kinase inhibitors (e.g., 6-dimethylaminopurine (DMAP)); temperature change; protein synthesis inhibitors (e.g., cyclohexamide); phorbol esters such as phorbol 12-myristate 13-acetate (PMA); mechanical techniques; and thapsigargin. The invention includes any activation techniques known in the art. See, e.g., U.S. Pat. No. 5,496,720, entitled “Parthenogenic Oocyte Activation” to Susko-Parrish et al., issued on Mar. 5, 1996; and U.S. patent application Ser. No. 09/176,395, filed on Oct. 21, 1998, each of which is incorporated by reference herein in its entirety, including all figures, tables, and drawings.

The term “fusion” as used herein refers to the combination of portions of lipid membranes corresponding to the totipotent mammalian cell nuclear donor and the recipient oocyte. Lipid membranes can correspond to the plasma membranes of cells or nuclear membranes, for example. The fusion can occur between the nuclear donor and recipient oocyte when they are placed adjacent to one another, or when the nuclear donor is placed in the perivitelline space of the recipient oocyte, for example. Specific examples for translocation of the totipotent mammalian cell into the oocyte are described hereafter in other preferred embodiments. These techniques for translocation are fully described in the references cited previously herein in reference to nuclear transfer.

The term “electrical pulses” as used herein refers to subjecting the nuclear donor and recipient oocyte to electric current. For nuclear transfer, the nuclear donor and recipient oocyte can be aligned between electrodes and subjected to electrical current. The electrical current can be alternating current or direct current. The electrical current can be delivered to cells for a variety of different times as one pulse or as multiple pulses. The cells are typically cultured in a suitable medium for the delivery of electrical pulses. Examples of electrical pulse conditions utilized for nuclear transfer are described in the references and patents previously cited herein in reference to nuclear transfer.

The term “fusion agent” as used herein refers to any compound or biological organism that can increase the probability that portions of plasma membranes from different cells will fuse when a totipotent mammalian cell nuclear donor is placed adjacent to the recipient oocyte. In preferred embodiments fusion agents are selected from the group consisting of polyethylene glycol (PEG), trypsin, dimethylsulfoxide (DMSO), lectins, agglutinin, viruses, and Sendai virus. These examples are not meant to be limiting and other fusion agents known in the art are applicable and included herein.

The term “suitable concentration” as used herein in reference to fusion agents, refers to any concentration of a fusion agent that affords a measurable amount of fusion. Fusion can be measured by multiple techniques well known to a person of ordinary skill in the art, such as by utilizing a light microscope, dyes, and fluorescent lipids, for example.

For the purposes of the present invention, the term “embryo” or “embryonic” as used herein refers to a developing cell mass that has not implanted into the uterine membrane of a maternal host. Hence, the term “embryo” as used herein can refer to a fertilized oocyte, a cybrid (defined herein), a pre-blastocyst stage developing cell mass, and/or any other developing cell mass that is at a stage of development prior to implantation into the uterine membrane of a maternal host. Embryos of the invention may not display a genital ridge. Hence, an “embryonic cell” is isolated from and/or has arisen from an embryo.

An embryo can represent multiple stages of cell development. For example, a one cell embryo can be referred to as a zygote, a solid spherical mass of cells resulting from a cleaved embryo can be referred to as a morula, and an embryo having a blastocoel can be referred to as a blastocyst.

In preferred embodiments (1) an embryo of the present invention is a mammalian embryo; (2) a mammalian embryo is selected from the group consisting of canid embryos, felid embryos, murid embryos, leporid embryos, ursid embryos, mustelid embryos, and human and non-human primate embryos; (3) a mammalian embryos is an ungulate embryo; and (4) an ungulate embryo is selected from the group consisting of suid embryos, ovid embryos, equid embryos, bovid embryos, caprid embryos, and cervid embryos.

The terms “fetus” and “fetal” as used herein refers to a developing cell mass that has implanted into the uterine membrane of a maternal host. A fetus can include such defining features as a genital ridge, for example. A genital ridge is a feature easily identified by a person of ordinary skill in the art, and is a recognizable feature in fetuses of most animal species. The term “fetal cell” as used herein can refer to any cell isolated from and/or has arisen from a fetus or derived from a fetus. The term “non-fetal cell” is a cell that is not derived or isolated from a fetus.

In preferred embodiments (1) a fetus of the present invention is a mammalian fetus; (2) a mammalian fetus is selected from the group consisting of canid fetuses, felid fetuses, murid fetuses, leporid fetuses, ursid fetuses, mustelid fetuses, and human and non-human primate fetuses; (3) a mammalian fetus is an ungulate fetus; and (4) an ungulate fetus is selected from the group consisting of suid fetuses, ovid fetuses, equid fetuses, bovid fetuses, caprid fetuses, and cervid fetuses.

Additional embodiments relate to methods for preparing gene expression databases comprising two or more, and up to numbers of 10,000 or more, expressed sequence tags, the expression of which can be used to identify cells, embryos, or fetuses as being developmentally competent or developmentally incompetent.

In preferred embodiments, gene expression databases can be prepared by comparing one or more first nucleic acid molecules obtained from one or more embryos produced by nuclear transfer using a developmentally competent nuclear donor cell to one or more second nucleic acid molecules obtained from one or more embryos produced by nuclear transfer using a developmentally incompetent nuclear donor cell, and identifying one or more nucleic acid molecules that are present in the population of first nucleic acid molecules, but that are not present at a detectable level in the population of second nucleic acid molecules, to provide two or more expressed sequence tags. ESTs (or their complementary sequences) so identified can then be combined in a gene expression database. Cells, embryos, and fetuses can be identified as developmentally competent based on the presence of at least one of the ESTs in such a gene expression database. In particularly preferred embodiments, a cell, embryo or fetus is identified as developmentally competent based on the presence of at least about 75% of the ESTs in such a gene expression database; at least about 90% of the ESTs in such a gene expression database; at least about 95% of the ESTs in such a gene expression database; and about 100% of the ESTs in such a gene expression database.

Similarly, in other preferred embodiments, gene expression databases can be prepared by comparing one or more first nucleic acid molecules obtained from one or more embryos produced by nuclear transfer using a developmentally incompetent nuclear donor cell to one or more second nucleic acid molecules obtained from one or more embryos produced by nuclear transfer using a developmentally competent nuclear donor cell, and identifying one or more nucleic acid molecules that are present in the population of first nucleic acid molecules, but that are not present at a detectable level in the population of second nucleic acid molecules, to provide two or more expressed sequence tags. ESTs (or their complementary sequences) so identified can then be combined in a gene expression database. Cells, embryos, and fetuses can be identified as developmentally incompetent based on the presence of at least one of the ESTs in such a gene expression database. In particularly preferred embodiments, a cell, embryo or fetus is identified as developmentally incompetent based on the presence of at least about 75% of the ESTs in such a gene expression database; at least about 90% of the ESTs in such a gene expression database; at least about 95% of the ESTs in such a gene expression database; and about 100% of the ESTs in such a gene expression database.

Most preferably, gene expression databases can be prepared by comparing one or more first nucleic acid molecules obtained from one or more embryos produced by nuclear transfer using a developmentally competent nuclear donor cell to one or more second nucleic acid molecules obtained from one or more embryos produced by nuclear transfer using a developmentally incompetent nuclear donor cell, and identifying one or more ESTs that are present in the population of first nucleic acid molecules, but that are not present at a detectable level in the population of second nucleic acid molecules, and one or more ESTs that are present in the population of second nucleic acid molecules, but that are not present at a detectable level in the population of first nucleic acid molecules. ESTs (or their complementary sequences) so identified can then be combined in a gene expression database.

In particularly preferred embodiments, the comparing step comprises comparing one or more nucleic acid molecules to a reference nucleic acid library, preferably obtained from the same species as the source of the nucleic acid molecules. The term “nucleic acid library” as used herein refers to a collection of DNA molecules derived from and representing all or part of the genetic material of an organism, tissue, or cell. Examples of nucleic acid libraries are genomic libraries, which are derived from restriction fragments of a genome, and cDNA libraries, which are derived from the mRNA of an organism, tissue, or cell. In preferred embodiments, nucleic acid libraries can be developmentally specific, i.e., derived from a specific developmental stage, cell lineage specific, i.e., derived from a specific cell lineage, and/or tissue specific, i.e., derived from a specific tissue.

In yet other embodiments, the invention relates to methods for identifying a developmentally competent nuclear donor cell line, using the ESTs and gene expression databases of the invention.

These methods can comprise: performing one or more nuclear transfer procedures using cells(s) separated from a cell line to provide one or more nuclear transfer embryos; culturing each of the nuclear transfer embryos to at least two cells; separating at least one cell from each of the cultured embryos; determining the developmental competence of each of the separated embryonic cells by comparing one or more nucleic acid molecules from each embryonic cell to a gene expression database; and identifying those embryos resulting from nuclear transfer of a developmentally competent nuclear donor cell.

The term “separating” as used herein refers to isolating one or more cells from a cell mass or cell culture. Cells can be separated by mechanical and chemical means well known to the skilled artisan. Cells can also be separated, for example, by biopsy or needle aspiration of a cell mass or cell culture. In this context, a “cell mass” can refer to an embryo, a fetus, or an animal.

The term “determining the developmental competence” with respect to embryos refers to identifying if an embryo is capable of developing into a live born animal. In certain embodiments, developmental competence is determined by implanting an embryo in a maternal host and allowing the embryo to develop until it either terminates or results in a live born animal. In certain other embodiments, developmental competence is determined by comparing the nucleic acid sequences present and/or expressed in one or more cells of an embryo to one or more nucleic acids that identify a cell as “developmentally competent” or “developmentally incompetent.” Preferably, this determination is made using the ESTs and gene expression databases described herein. Cells obtained from an embryo can be used directly to determine the developmental competence of the source embryo, or the cells can be cultured prior to their use.

In preferred embodiments, an embryo is determined to be developmentally competent based on the presence in one or more cells obtained from the embryo of one or more nucleic acid sequences that are known to be present and/or expressed in a cell line that has been demonstrated to be developmentally competent, but that are present and/or expressed at a reduced or nondetectable level in a cell line that has been tested for, but has failed to demonstrate developmental competence. As discussed herein, expression of nucleic acid sequences in a cell may be indirectly detected by detecting molecules produced from the nucleic acid sequences, such as proteins or peptides.

In other preferred embodiments, an embryo is determined to be developmentally incompetent based on the presence in a cell obtained from the embryo of one or more nucleic acid sequences that are that are present and/or expressed in a cell line that has been tested for, but has failed to demonstrate developmental competence, but that are present and/or expressed at a reduced or nondetectable level in a cell line that has been demonstrated to be developmentally competent.

In yet other preferred embodiments, an embryo is determined to be developmentally competent based the presence in a cell obtained from the embryo of one or more nucleic acid sequences that are known to be present and/or expressed in a cell line that has been demonstrated to be developmentally competent, but that are present and/or expressed at a reduced or nondetectable level in a cell line that has been tested for, but has failed to demonstrate developmental competence; and the absence of one or more nucleic acid sequences that are that are present and/or expressed in a cell line that has been tested for, but has failed to demonstrate developmental competence, but that are present and/or expressed at a reduced or nondetectable level in a cell line that has been demonstrated to be developmentally competent.

In additional preferred embodiments, an embryo is determined to be developmentally incompetent based the presence in a cell obtained from the embryo of one or more nucleic acid sequences that are that are present and/or expressed in a cell line that has been tested for, but has failed to demonstrate developmental competence, but that are present and/or expressed at a reduced or nondetectable level in a cell line that has been demonstrated to be developmentally competent; and the absence of one or more nucleic acid sequences that are known to be present and/or expressed in a cell line that has been demonstrated to be developmentally competent, but that are present and/or expressed at a reduced or nondetectable level in a cell line that has been tested for, but has failed to demonstrate developmental competence.

In additional embodiments, the invention relates to methods for producing one or more embryos, fetuses, and/or animals by nuclear transfer procedures using a developmentally competent nuclear donor cell.

Methods for preparing embryos according to the invention preferably comprise: performing one or more nuclear transfer procedures using cells(s) separated from a cell line to provide one or more nuclear transfer embryos; culturing each of the nuclear transfer embryos to at least two cells; separating at least one cell from each of the cultured embryos; determining the developmental competence of each of the separated embryonic cells by comparing one or more nucleic acid molecules from each embryonic cell to a gene expression database; and identifying those embryos resulting from nuclear transfer of a developmentally competent nuclear donor cell. Preferred methods for preparing fetuses and/or animals further comprise implanting the identified embryos into one or more recipient females for development into one or more fetuses and/or animals.

The term “culturing” as used herein in reference to embryos refers to laboratory procedures that involve placing an embryo in a culture medium. An embryo can be placed in a culture medium for an appropriate amount of time to allow stasis of an embryo, or to allow the embryo to grow in the medium. Culture media suitable for culturing embryos are well-known to those skilled in the art. See, e.g., Nagashima et al., 1997, Mol. Reprod. Dev. 48: 339-343; Petters & Wells, 1993, J. Reprod. Fert. (Suppl) 48: 61-73; Reed et al., 1992, Theriogenology 37: 95-109; Dobrinsky et al., 1996, Biol. Reprod. 55: 1069-1074; U.S. Pat. No. 5,213,979, First et al., “In Vitro Culture of Bovine Embryos,” May 25, 1993; U.S. Pat. No. 5,096,822, Rosenkrans, Jr. et al., “Bovine Embryo Medium,” Mar. 17, 1992, each of which is incorporated herein by reference in its entirety, including all figures, tables, and drawings. Alternatively, an embryo may be “cultured in vivo,” for example by placing the embryo into the ligated oviduct of a recipient female, for an appropriate amount of time to allow stasis of an embryo, or to allow the embryo to grow. Techniques of culturing an embryo in vivo are well known to those skilled in the art.

The term “suitable medium” as used herein refers to any medium that allows cell proliferation or allows stasis of an embryo. If a medium allows cell proliferation, a suitable medium need not promote maximum proliferation, only measurable cell proliferation. A suitable medium for embryo development can be an embryo culture medium described herein by example. Embryos of the invention can be cultured in media with or without feeder cells. In preferred embodiments, the feeder cells can be cumulus cells.

The terms “maternal recipient” and “recipient female” as used herein refers to a female animal which is implanted with an embryo for development of the embryo. A maternal recipient may be either homospecific or xenospecific to the implanted embryo. For example it has been shown in the art that bovine embryos can develop in the oviducts of sheep. Stice & Keefer, 1993, “Multiple generational bovine embryo cloning,” Biology of Reproduction 48: 715-719. Implanting techniques are well known to a person of ordinary skill in the art. See, e.g., Polge & Day, 1982, “Embryo transplantation and preservation,” Control of Pig Reproduction, DJA Cole and GR Foxcroft, eds., London, UK, Butterworths, pp. 227-291; Gordon, 1997, “Embryo transfer and associated techniques in pigs,” Controlled reproduction in pigs (Gordon, ed), CAB International, Wallingford UK, pp 164-182; and Kojima, 1998, “Embryo transfer,” Manual of pig embryo transfer procedures, National Livestock Breeding Center, Japanese Society for Development of Swine Technology, pp 76-79, each of which is incorporated herein by reference in its entirety, including all figures, tables, and drawings.

In preferred embodiments (1) an embryo, fetus, or animal of the present invention is a mammalian embryo, fetus, or animal; (2) a mammal is selected from the group consisting of canids, felids, murids, leporids, ursids, mustelids, and human and non-human primates; (3) a mammal is an ungulate; and (4) an ungulate is selected from the group consisting of suids, ovids, equids, bovids, caprids, and cervids.

In particularly preferred embodiments, embryos, fetuses and/or animals of the invention are transgenic embryos, fetuses and/or animals. The term “transgenic” as used herein in reference to embryos, fetuses and animals refers to an embryo, fetus or animal comprising one or more cells whose genomes has been altered using recombinant DNA techniques. In preferred embodiments, a transgenic embryo, fetus, or animal comprises one or more transgenic cells. While germ line transmission is not a requirement of transgenic embryos, fetuses, or animals as that term is used herein, in particularly preferred embodiments a transgenic embryo, fetus, or animal can pass its transgenic characteristic(s) through the germ line. In certain embodiments, a transgenic embryo, fetus or animal expresses one or more exogenous genes as exogenous RNA and protein molecules. Most preferably, a transgenic embryo, fetus or animal results from a nuclear transfer procedure using a transgenic nuclear donor cell.

Additional embodiments relate to methods for assessing the effect of one or more changes to a nuclear transfer protocol by comparing the developmental competence of nuclear transfer embryos resulting from the changed protocol to the developmental competence of nuclear transfer embryos resulting from a baseline protocol, using the ESTs and expression databases of the invention

Preferably, changes in a nuclear transfer protocol are assessed by: performing one or more nuclear transfer procedures according to a first nuclear transfer protocol to produce one or more first protocol nuclear transfer embryos; performing one or more nuclear transfer procedures according to a second nuclear transfer protocol comprising one or more changes to said first nuclear transfer protocol, to produce one or more second protocol nuclear transfer embryos; determining the developmental competence of each of the first protocol and second protocol nuclear transfer embryos; and assessing the effect of the changes to the protocol by comparing the developmental competence of the first protocol nuclear transfer embryos to the developmental competence of the second protocol nuclear transfer embryos.

The term “assessing the effect of one or more changes in a nuclear transfer protocol” as used herein refers to the process of determining whether changing one or more variables in a nuclear transfer protocol alters the developmental competence of nuclear transfer embryos produced by the protocol. The skilled artisan will understand that the number of variables which may be changed are myriad, and can include changing the donor cell medium composition, the activation parameters, the fusion parameters, the embryo culture parameters, etc. By comparing the percentage of developmentally competent embryos produced by a baseline protocol to the percentage of developmentally competent embryos produced by the changed protocol, the effect of the changes can be determined. In preferred embodiments, the effect of the changes to the protocol is to increase the percentage of developmentally competent embryos produced. In certain embodiments, the effect of the changes to the protocol is to decrease the percentage of developmentally competent embryos produced.

The term “comparing the developmental competence” as used herein in reference to embryos refers to determining the percentage of developmentally competent embryos in two different group of embryos, and comparing the relative percentages in the two groups. The term “determining the developmental competence” of embryos is defined herein. In preferred embodiments, a group of embryos for comparison purposed comprise at least 2 embryos, at least 3 embryos, at least 4 embryos, at least 5 embryos, at least 6 embryos, at least 7 embryos, at least 8 embryos, at least 9 embryos, at least 10 embryos, at least 15 embryos, at least 20 embryos, at least 25 embryos, at least 30 embryos, at least 40 embryos, at least 50 embryos, at least 60 embryos, at least 70 embryos, at least 100 embryos, at least 200 embryos, at least 300 embryos, at least 400 embryos, and at least 500 embryos.

In further embodiments, the invention relates to nucleic acid arrays comprising the ESTs and gene expression libraries of the invention that can be used in methods, such as those described herein, to identify cells, embryos, or fetuses as being developmentally competent or developmentally incompetent.

The term “nucleic acid array” as used herein refers to one or more nucleic acid molecules affixed to a solid matrix. In certain embodiments, nucleic acid arrays can be used as solid supports for hybridization assays. Suitable solid matrices for attaching nucleic acids, and methods of attachment are well known in the art. See, e.g., U.S. Pat. No. 6,004,755, issued on Dec. 21, 1999 to B. Wang, entitled “Quantitative Microarray Hybridization Assays;” U.S. Pat. No. 5,861,242, issued on Jan. 19, 1999 to Chee et al., entitled “Array of Nucleic Acid Probes on Biological Chips for Diagnosis of HIV and Methods of Using the Same;” U.S. Pat. No. 5,830,645, issued on Nov. 3, 1998 to Pinkel et al., entitled “Comparative Fluorescence Hybridization to Nucleic Acid Arrays;” U.S. Pat. No. 5,667,976, issued Sep. 16, 1997 to Van Ness et al., entitled “Solid Supports for Nucleic Acid Hybridization Assays;” and U.S. Pat. No. 5,215,882, issued on Jun. 1, 1993 to Bahl et al., entitled “Method of Immobilizing Nucleic Acid on a Solid Surface for Use in Nucleic Acid Hybridization Assays,” each of which is incorporated in its entirety, including all tables, figures, and claims. In preferred embodiments, a solid phase can be papers, nitrocellulose membranes, nylon membranes, glass, magnetic materials, magnetic beads, polymeric beads, or silicon surfaces. In other preferred embodiments a solid phase can be a solid or semisolid polymer such as polyacrylamide gels and agarose gels.

Preferably, a nucleic acid array comprises at least one nucleic acid molecule, the expression of which (or its complementary sequence) identifies a cell as being developmentally competent or developmentally incompetent. More preferably, a nucleic acid array comprises from 2 to 10,000 or more nucleic acid molecules, the expression of which (or their complementary sequences) identifies a cell as being developmentally competent or developmentally incompetent. In particularly preferred embodiments, a nucleic acid array comprises at least one nucleic acid molecule, the expression of which (or its complementary sequence) identifies a cell as being developmentally competent, and at least one nucleic acid molecule, the expression of which (or its complementary sequence) identifies a cell as being developmentally incompetent.

In particularly preferred embodiments, a cell, embryo or fetus is identified as developmentally competent based on the presence of complementary sequences to at least about 75% of the ESTs comprised in such a nucleic acid array; at least about 90% of the ESTs comprised in such a nucleic acid array; at least about 95% of the ESTs comprised in such a nucleic acid array; and about 100% of the ESTs comprised in such a nucleic acid array.

In a second aspect, the invention concerns identifying and using one or more expressed sequence tags, the expression of which can be used to identify a cell, most preferably a stem cell, as being capable of committing to a specific cell lineage.

In certain embodiments, cells, and most preferably stem cells, can be identified as being capable of committing to a specific cell lineage based on the expression of an expressed sequence tag (or its complementary sequence) known to be present and/or expressed in a cell line that has been demonstrated to be capable of committing to that cell lineage, but that is present and/or expressed at a reduced or nondetectable level in a cell line that has been tested for, but has failed to demonstrate such a capability. Similarly, cells can be identified as being incapable of committing to a specific cell lineage based on the expression of an expressed sequence tag (or its complementary sequence) known to be present and/or expressed in a cell line that has been tested for, but has failed to demonstrate the capability of committing to the cell lineage, but that is present and/or expressed at a reduced or nondetectable level in a cell line that has been demonstrated to be capable of committing to that cell lineage.

The term “stem cell” as used herein refers to one or more cells capable of differentiating into one or more different cell lineages. For example, hematopoetic stem cells can differentiate into one or more different blood cell types such as erythrocytes, platelets, macrophages, lymphocytes, etc. Such cells are pluripotent. Alternatively, some stem cells differentiate into a single cell lineage. For example, epidermal stem cells can differentiate into cornified epidermal cells. Such cells are unipotent. In particularly preferred embodiments, a stem cell is an embryonic stem cell.

The term “embryonic stem cell” as used herein refers to pluripotent cells isolated from an embryo that are maintained in in vitro cell culture. Embryonic stem cells may be, but need not be, totipotent. Embryonic stem cells may be cultured with or without feeder cells. Embryonic stem cells can be established from embryonic cells isolated from embryos at any stage of development, including blastocyst stage embryos and pre-blastocyst stage embryos. Embryonic stem cells may have a rounded cell morphology and may grow in rounded cell clumps on feeder layers. Embryonic stem cells are well known to a person of ordinary skill in the art. See, e.g., WO 97/37009, entitled “Cultured Inner Cell Mass Cell-Lines Derived from Ungulate Embryos,” Stice and Golueke, published Oct. 9, 1997, and Yang & Anderson, 1992, Theriogenology 38: 315-335, each of which is incorporated herein by reference in its entirety, including all figures, tables, and drawings. See, also, e.g., Piedrahita et al., 1998, Biol. Reprod. 58: 1321-1329; Wianny et al., 1997, Biol. Reprod. 57: 756-764; Moore & Piedrahita, 1997, In Vitro Cell Biol. Anim. 33: 62-71; Moore, & Piedrahita, 1996, Mol. Reprod. Dev. 45: 139-144; Wheeler, 1994, Reprod. Fert. Dev. 6: 563-568; Hochereau-de Reviers & Perreau, Reprod. Nutr. Dev. 33: 475-493; Strojek et al., 1990, Theriogenology 33: 901-903; Piedrahita et al., 1990, Theriogenology 34: 879-901; and Evans et al., 1990, Theriogenology 33: 125-129, each of which is incorporated herein by reference in its entirety, including all figures, tables, and drawings.

In preferred embodiments (1) a stem cell or a stem cell line of the present invention is a mammalian stem cell or stem cell line; (2) a mammalian stem cell or stem cell line is selected from the group consisting of canid stem cells or stem cell lines, felid stem cells or stem cell lines, murid stem cells or stem cell lines, leporid stem cells or stem cell lines, ursid stem cells or stem cell lines, mustelid stem cells or stem cell lines, and human and non-human primate stem cells or stem cell lines; (3) a mammalian stem cells or stem cell lines is an ungulate stem cells or stem cell lines; and (4) an ungulate stem cells or stem cell lines is selected from the group consisting of suid stem cells or stem cell lines, ovid stem cells or stem cell lines, equid stem cells or stem cell lines, bovid stem cells or stem cell lines, caprid stem cells or stem cell lines, and cervid stem cells or stem cell lines.

The term “differentiated” as used herein refers to a cell that has developed from an unspecialized phenotype to a specialized phenotype.

The term “undifferentiated cell” as used herein refers to a precursor cell that has an unspecialized phenotype and is capable of differentiating. An example of an undifferentiated cell is a stem cell.

The term “committing to a specific cell lineage” as used herein refers to the ability of a cell to differentiate into a specific cell type. For example, a hematopoetic stem cell may be capable of committing to an erythrocyte, platelet, macrophage, lymphocyte, etc., lineage, while an embryonic stem cell may be capable of committing to a wider variety of cell lineages, such as a muscular, neuronal, hematopoetic, osteal, germinal, etc., cell lineage. A cell, and in particular a stem cell, may be capable of committing to certain cell lineages, yet incapable of committing to others. A cell may only commit to a specific cell lineage when exposed to a proper differentiation-inducing stimulus.

As discussed above, the term “identifies” or “identifying” as used herein with respect to cells refers to the ability to distinguish between cells having two distinct characteristics. In preferred embodiments, a cell or cell line that is capable of committing to a specific cell lineage can be distinguished from that is not capable of committing to that lineage. In certain preferred embodiments, an EST or ESTs identify a cell or cell line as “capable of committing to a specific cell lineage” if the EST sequences are present and/or expressed in stem cells known to be capable of committing to that lineage, but are present and/or expressed at a reduced or nondetectable level in stem cells that have been tested for, but have failed to demonstrate the ability to commit to that lineage. Similarly, an EST or ESTs identify a cell or cell line as “incapable of committing to a specific cell lineage” if the sequences are present and/or expressed in embryos in stem cells that have been tested for, but have failed to demonstrate the ability to commit to that lineage, but are present and/or expressed at a reduced or nondetectable level in known to be capable of committing to that lineage.

In additional embodiments, expressed sequence tags can be grouped in numbers of 2 or more, and up to numbers of 10,000 or more, to provide a gene expression database. The expression of one or more expressed sequence tags in the database can be used to identify cells, most preferably stem cells, capable of committing to a specific cell lineage.

Preferably, a gene expression database comprises two or more expressed sequence tags (or their complementary sequences) known to be present and/or expressed in a cell line that has been demonstrated to be capable of committing to a specific cell lineage, but that are present and/or expressed at a reduced or nondetectable level in a cell line that has been tested for, but has failed to demonstrate such a capability. Cells can be identified as capable of committing to a specific cell lineage based on the presence of at least one of the ESTs in such a gene expression database. In particularly preferred embodiments, cells can be identified as capable of committing to a specific cell lineage based on the presence of at least about 75% of the ESTs in such a gene expression database; at least about 90% of the ESTs in such a gene expression database; at least about 95% of the ESTs in such a gene expression database; and about 100% of the ESTs in such a gene expression database.

Likewise, a gene expression database preferably comprises two or more expressed sequence tags (or their complementary sequences) known to be present and/or expressed in a cell line that has been tested for, but has failed to demonstrate the capability of committing to a specific cell lineage, but that are present and/or expressed at a reduced or nondetectable level in a cell line that has been demonstrated to be capable of committing to that cell lineage. Cells can be identified as incapable of committing to a specific cell lineage based on the presence of at least one of the ESTs in such a gene expression database. In particularly preferred embodiments, cells can be identified as incapable of committing to a specific cell lineage based on the presence of at least about 75% of the ESTs in such a gene expression database; at least about 90% of the ESTs in such a gene expression database; at least about 95% of the ESTs in such a gene expression database; and about 100% of the ESTs in such a gene expression database.

Most preferably, a gene expression database comprises at least one EST (or its complementary sequence) known to be present and/or expressed in a cell line that has been demonstrated to be capable of committing to a specific cell lineage, but that is present and/or expressed at a reduced or nondetectable level in a cell line that has been tested for, but has failed to demonstrate such a capability; and at least one EST (or its complementary sequence) known to be present and/or expressed in a cell line that has been tested for, but has failed to demonstrate the capability of committing to a specific cell lineage, but that is present and/or expressed at a reduced or nondetectable level in a cell line that has been demonstrated to be capable of committing to that cell lineage. In such embodiments, cells can be identified as capable of committing to a specific cell lineage based on the presence on the presence of at least one of the former ESTs, and the absence of the latter ESTs. Likewise, cells can be identified as incapable of committing to a specific cell lineage based on the presence of at least one of the latter ESTs, and the absence of the former ESTs.

In further embodiments, the invention concerns methods for identifying one or more expressed sequence tags, the expression of which can be used to identify cells, most preferably stem cells, capable of committing to a specific cell lineage.

In preferred embodiments, one or more ESTs are identified by comparing one or more first nucleic acid molecules obtained from one or more cell lines that have been demonstrated to be capable of committing to a specific cell lineage to one or more second nucleic acid molecules obtained from one or more cell lines that have been tested for, but have failed to demonstrate such a capability. ESTs that signal the capability to commit to a specific cell lineage are identified as one or more nucleic acid molecules that are present in the population of first nucleic acid molecules, but that are not present at a detectable level in the population of second nucleic acid molecules. Likewise, one or more nucleic acid molecules that are present in the population of second nucleic acid molecules, but that are not present at a detectable level in the population of first nucleic acid molecules are identified as ESTs that signal the incapability to commit to a specific cell lineage.

In particularly preferred embodiments, an EST that signals the capability to commit to a specific cell lineage is a nucleic acid molecule (1) present and/or expressed in at least about 75% of cell lines that have been demonstrated to be capable of committing to a specific cell lineage, but that is present and/or expressed at a reduced or nondetectable level in at least about 75% of cell lines that have been tested for, but have failed to demonstrate such a capability; (2) present and/or expressed in at least about 90% of cell lines that have been demonstrated to be capable of committing to a specific cell lineage, but that is present and/or expressed at a reduced or nondetectable level in at least about 90% of cell lines that have been tested for, but have failed to demonstrate such a capability; (3) present and/or expressed in at least about 95% of cell lines that have been demonstrated to be capable of committing to a specific cell lineage, but that is present and/or expressed at a reduced or nondetectable level in at least about 95% of cell lines that have been tested for, but have failed to demonstrate such a capability; and (4) present and/or expressed in at least about 100% of cell lines that have been demonstrated to be capable of committing to a specific cell lineage, but that is present and/or expressed at a reduced or nondetectable level in at least about 100% of cell lines that have been tested for, but have failed to demonstrate such a capability.

In other particularly preferred embodiments, an EST that signals the incapability to commit to a specific cell lineage is a nucleic acid molecule (1) present and/or expressed in at least about 75% of cell lines that have been tested for, but have failed to demonstrate the ability to commit to a specific cell lineage, but that is present and/or expressed at a reduced or nondetectable level in at least about 75% of cell lines that have been demonstrated to be capable of committing to a specific cell lineage; (2) present and/or expressed in at least about 90% of cell lines that have been tested for, but have failed to demonstrate the ability to commit to a specific cell lineage, but that is present and/or expressed at a reduced or nondetectable level in at least about 90% of cell lines that have been demonstrated to be capable of committing to a specific cell lineage; (3) present and/or expressed in at least about 95% of cell lines that have been tested for, but have failed to demonstrate the ability to commit to a specific cell lineage, but that is present and/or expressed at a reduced or nondetectable level in at least about 95% of cell lines that have been demonstrated to be capable of committing to a specific cell lineage; and (4) present and/or expressed in at least about 100% of cell lines that have been tested for, but have failed to demonstrate the ability to commit to a specific cell lineage, but that is present and/or expressed at a reduced or nondetectable level in at least about 100% of cell lines that have been demonstrated to be capable of committing to a specific cell lineage.

In another aspect, the invention concerns methods that identify one or more molecules that affect the developmental competence of cells, cell lines, embryos, fetuses, and/or animals.

In certain embodiments, molecules can be identified that induce developmental competence in an otherwise developmentally incompetent cell line. Similarly, molecules can be identified that induce developmental incompetence in an otherwise developmentally competent cell line. Such molecules can be used to increase the availability of developmentally competent cells for use as nuclear donor cells in nuclear transfer procedures, for the treatment of certain diseases, or for preventing full term pregnancies.

In preferred embodiments, molecules that induce developmental competence in an otherwise developmentally incompetent cell line can be identified by: contacting a developmentally incompetent cell line with one or more molecules to provide a treated cell line; separating one or more cells from the treated cell line to provide one or more separated cells; performing one or more nuclear transfer procedures using one or more separated cells to provide one or more nuclear transfer embryos; and determining the developmental competence of each of the nuclear transfer embryos. In particularly preferred embodiments, developmental competence is determined by comparing a plurality of nucleic acid molecules obtained from each of the embryos to a gene expression database of the instant invention.

Likewise, molecules that induce developmental incompetence in an otherwise developmentally competent cell line can be identified by: contacting a developmentally competent cell line with one or more molecules to provide a treated cell line; separating one or more cells from the treated cell line to provide one or more separated cells; performing one or more nuclear transfer procedures using one or more separated cells to provide one or more nuclear transfer embryos; and determining the developmental competence of each of the nuclear transfer embryos. In particularly preferred embodiments, developmental competence is determined by comparing a plurality of nucleic acid molecules obtained from each of the embryos to a gene expression database of the instant invention.

The term “contacting” as used herein with respect to cells refers to bringing one or more cells together with one or more molecules, whether in an in vitro system (e.g., in a test tube or an ex vivo system) or an in vivo system. One or more cells may be removed from an organism for contacting with one or more molecules, and then the cells can be returned to the same or a different animal.

In further embodiments, one or more molecules identified as inducing or inhibiting developmental competence can be used to induce or inhibit developmental competence in cells, cell lines, embryos, fetuses, or animals, by administering one or more molecules so identified to cells, cell lines, embryos, fetuses, or animals. In particularly preferred embodiments, administering one or more molecules so identified can be used to treat diseases in an animal, embryo, or fetus, or to prevent a full term pregnancy.

The term “administering” as used herein refers to a method of contacting one or more molecules with the one or more cells, cell lines, embryos, fetuses, or animals. In the case of embryos, fetuses, and animals, cells may be contacted with one or more molecules while within an embryo, fetus, or animal; or cells may be removed from the embryo, fetus, or animal, contacted with one or more molecules, and then returned to the same or a different embryo, fetus, or animal. The compound can be prepared using a carrier such as dimethyl sulfoxide (DMSO) in an aqueous solution or preparation. The compounds may be administered to cells or tissues using a suitable buffered solution.

Cells existing outside an organism can be maintained or grown in cell culture dishes. For cells harbored within an organism, many techniques exist in the art to administer compounds, including (but not limited to) oral, parenteral, dermal, ocular, subcutaneous, and rectal applications. For cells outside of the organism, multiple techniques exist in the art to administer the compounds, including (but not limited to) cell microinjection techniques, transformation techniques, and carrier techniques.

In particularly preferred embodiments, one or more molecules can be administered to one or more cultured or non-cultured embryonic cells, embryonic stem cells, inner cell mass cells, fetal cells, embryonic germ cells, somatic cells, adult cells, neurons, glial cells, muscle cells, bone marrow cells, stem cells, hepatocytes, renal cells, muscle cells, cardiac cells, epidermal cells, oocytes, fertilized oocytes, spermatocytes, nuclear transfer embryos, pancreatic cells, lymphocytes, tumor cells, malignant cells, teratoma cells, seminoma cells, carcinoma cells, lymphoma cells, glioblastoma cells, hepatocellular carcinoma cells, and hamartoma cells.

The term “pharmaceutically acceptable composition” refers to a preparation comprising one or more molecules. The composition is acceptable if it does not appreciably cause irritations to the organism administered the composition.

The term “suitable buffered solution” refers to an aqueous preparation of a molecule that comprises a salt that can control the pH of the solution at low concentrations. Because the salt exists at low concentrations, the salt preferably does not alter the function of cells.

In another aspect, the invention concerns methods that identify and use one or more molecules that induce lineage specific development in a cell line, most preferably a stem cell line.

In certain embodiments, molecules so identified can the be used to induce lineage-specific development in one or more cells, preferably stem cells or stem cell lines, that might otherwise be incapable of such development.

In preferred embodiments, molecules that induce lineage specific development in a cell line are identified by: contacting a stem cell line known to be capable of differentiation into a specific cell type with one or more molecules to provide a treated cell line; and determining the capability of the treated cell line to differentiate into a specific cell type. In particularly preferred embodiments, the capability of the treated cell line to differentiate into the cell type of interest is determined by comparing a plurality of nucleic acid molecules obtained from one or more treated cells to a gene expression database of the instant invention.

The summary of the invention described above is not limiting and other features and advantages of the invention will be apparent from the following detailed description of the preferred embodiments, as well as from the claims.

### BRIEF DESCRIPTION OF THE TABLES

Tables 1A and B illustrates data concerning the developmental competence of 59 different nuclear donor cell lines.

Table 2 illustrates EST sequences screened for differential expression in developmentally competent bovine embryos versus developmentally incompetent bovine embryos.

### BRIEF DESCRIPTION OF THE FIGURES

FIG. 1 illustrates comparisons of EG+ and ES− donor cell expression profiles, determined using cDNA microarray, differential display, and direct sequencing methods.

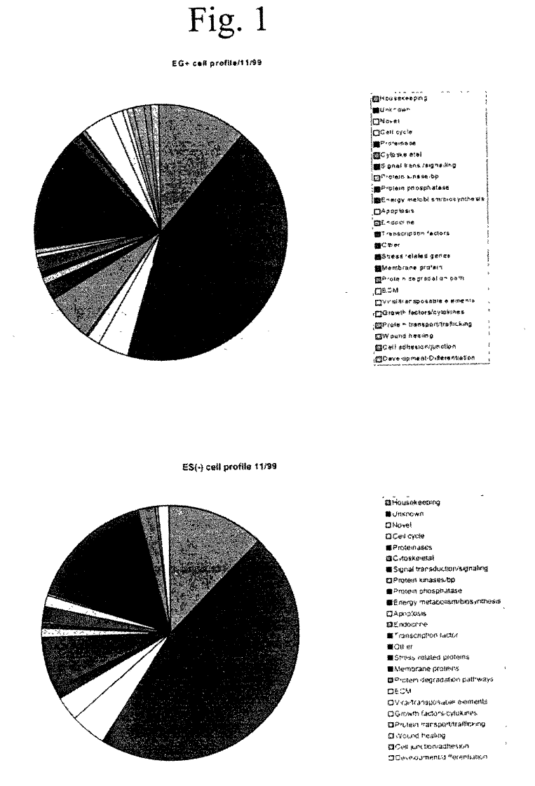

FIG. 2 illustrates immunoblot analysis of cultured EG+ and ES− donor cells.

FIG. 3 illustrates examples of differential display analyses comparing mRNA expression patterns in individual embryos prepared in vivo, or by nuclear transfer using EG+ and ES− donor cells.

FIG. 4A illustrates differential display analysis comapring banding patterns of 5 individual in vivo embryos, 6 individual IVF embryos, 5 individual NT embryos and the donor cell line (DC) used to reconstruct NT embryos. Arrows indicate bands present in all in vivo and at least 5 of 6 IVF produced embryos. FIG. 4B shows a histogram indicating the percentage of bands shared with in vivo embryos.

FIG. 5 illustrates identical cDNA arrays probed with mRNA representations of a single NT embryo (A) and a single in vivo embryo (B). Spots enclosed by circles represent clones detected at high levels in a single in vivo embryo and a single NT embryo reconstructed using a competent donor cell line, but at low levels (or undetected) in single NT embryos reconstructed from incompetent donor cell lines and an unknown cell line.

FIG. 6 illustrates a profile of the cDNA clones used for microarray analysis.

FIG. 7 illustrates cluster analysis performed on individual embryos prepared by nuclear transfer using developmentally competent and incompetent donor cell lines, and embryos prepared by nuclear transfer using donor cells obtained from a cell line of unknown developmental competence.

### DETAILED DESCRIPTION OF THE INVENTION

The present invention relates to materials and methods for evaluating and affecting the molecular events associated with cellular differentiation and reprogramming, and, in particular, for evaluating and affecting molecular events related to developmental competence and lineage-specific development. The invention provides numerous advantages over methods currently in use. For example, the methods described herein can dramatically increase the number of developmentally competent nuclear donor cells, oocytes, and embryos available. The methods described herein can also dramatically increase the efficiency of nuclear transfer procedures by identifying those nuclear donor cells, oocytes, and embryos most likely to result in successful live births, resulting in an increase in the number of viable embryos, fetuses, and live births, including transgenic embryos, fetuses, and animals. Moreover, the methods described herein can also dramatically increase the efficiency of nuclear transfer procedures by identifying techniques, such as oocyte and embryo maturation, oocyte activation, oocyte enucleation, timing of implantation, and maternal care most likely to result in successful live births.

As discussed herein, embryos produced by the methods described herein can be used in recloning procedures. Recloned embryos produced by such methods can exhibit enhanced developmental competence compared to embryos produced by a single round of nuclear transfer. In addition, recloning can enhance the efficiency of preparing transgenic embryos, fetuses and/or animals using gene targeting methods. Similarly, fetal cells (e.g., primordial germ cells) can be used as nuclear donor cells in multiple rounds of nuclear transfer for gene targeting methods. Following one or more rounds of nuclear transfer and genetic manipulation, cells obtained from the resulting embryos, fetuses, or animals exhibiting a gene targeting event (such as a knockout or a gene replacement) may be particularly useful as cell-based therapeutics.

Moreover, the materials and methods described herein can increase the efficiency at which cells, and particularly stem cells, can be induced to differentiate into a specific cell lineage. Particularly when coupled with the ability to perform gene targeting with increased efficiency, the instant methods can greatly foster development of cell-based therapeutics.

I. Obtaining and Using Tissue-Specific and Developmentally-Specific Marker Genes and Sequences

The instant invention describes methods to evaluate molecular events associated with cellular reprogramming and differentiation. The tissue-specific and developmentally-specific marker molecules described by the instant invention can be any molecules that are expressed differentially as cells undergo reprogramming to a developmentally competent state, or as cells commit to a specific differentiation pathway. Preferably, such marker molecules are nucleic acid molecules, such as mRNAs, or cDNAs obtained therefrom; however, downstream products of these nucleic acids, such as proteins resulting from translation of mRNAs, or products produced by those proteins, can also be associated with cellular reprogramming and differentiation by techniques well known to the skilled artisan.

A. Expressed Sequence Tags

Methods for identifying and isolating expressed sequence tags are well known to the ordinarily skilled artisan. mRNAs, or cDNAs prepared therefrom, are preferred as a source of expressed sequence tags, as these molecules represent the expressed subset of genomic nucleic acid sequences. Preferably, full length or partial length cDNA clones can be prepared from one or more cells, embryos, fetuses, tissues, or animals by methods such as those described in Sambrook, et al., 1989, Molecular Cloning: A Laboratory Manual, Second Edition, Cold Spring Harbor Press, Plainview, N.Y.; and Innis, et al, 1990, PCR Protocols: A Guide To Methods And Applications, Academic Press, San Diego, Calif. If necessary, RNA molecules that are present in only low abundance can be amplified by methods well known to those of skill in the art. See, e.g., Innis, et al., 1990.

B. Identifying Differentially Expressed Nucleic Acid Molecules

Tissue-specific and developmentally-specific nucleic acid molecules can be identified by comparing the mRNA or cDNA populations obtained from cells in two different differentiation or developmental states. Numerous methods are known to the skilled artisan for identifying commonly expressed and differentially expressed nucleic acid molecules. For example, northern analysis, nucleic acid sequencing, and S1 nuclease protection assays can be used to quantitate relative gene expression levels. Preferably, relative copy numbers of target nucleic acids can be determined as described in U.S. Pat. No. 5,830,645, issued to Pinkel et al. on Nov. 3, 1998, entitled “Comparative Fluorescence Hybridization to Nucleic Acid Arrays;” gene subtraction methods and differential display methods can identify sequences differeing in or common to two nucleic acid populations as described in U.S. Pat. No. 5,436,142, issued to Wigler et al. on Jul. 25, 1995, entitled “Methods for Producing Probes Capable of Distingushing Variant Genomic Sequences,” Liang and Pardee, 1997, Meth. Mol. Biol. 85: 3-11; and U.S. Pat. No. 5,935,788, issued to Burmer et al. on Aug. 10, 1999, entitled “Subtractive Hybridization Techniques for Identifying Differentially Expressed and Commonly Expressed Nucleic Acid;” and differential display PCR or RT-PCR can identify sequences differeing in or common to two nucleic acid populations as described in U.S. Pat. No. 5,773,213, issued to Gullans et al. on Jun. 30, 1998, entitled “Method for Conducting Sequential Nucleic Acid Hybridization Techniques.” Each of the references cited in this section are hereby incorporated by reference in their entirety, including all figures, tables, and claims.

C. Hybridization Supports

Nucleic acid hybridization techniques, such as those described herein, can be performed by methods that are well known to the ordinarily skilled artisan. See, e.g., Sambrook, et al., 1989, Molecular Cloning: A Laboratory Manual, Second Edition, Cold Spring Harbor Press, Plainview, N.Y.; and U.S. Pat. No. 5,215,882, issued to Bahl et al. on Jun. 1, 1993, entitled “Method of Immobilizing Nucleic Acid on a Solid Surface For Use In Nucleic Acid Hybridization Assays.” These methods can typically rely on affixing a test nucleic acid on a solid surface, such as cellulose or nylon papers or membranes or glass slides, which acts as a support for the hybridization assay. Numerous hybridization supports are known in the art. Particularly preferred hybridization supports are polymer beads and dipsticks, such as those described in U.S. Pat. No. 5,667,976, issued to Van Ness et al. on Sep. 16, 1997, entitled “Solid Supports for Nucleic Acid Hybridization Assays;” and nucleic acid arrays, macroarrays, and microarrays, such as those described in U.S. Pat. No. 5,861,242, issued to Chee et al. on Jan. 19, 1999, entitled “Array of Nucleic Acid Probes on Biolological Chips For Diagnosis of HIV and Methods of Using Same;” and U.S. Pat. No. 6,004,755, issued to Wang on Dec. 21, 1999, entitled “Quantitative Microarray Hybridization Assays.” Each of the references cited in this section are hereby incorporated by reference in their entirety, including all figures, tables, and claims.

D. Correlating Differentially Expressed Nucleic Acid Molecules to Cellular Reprogramming

The expression patterns of tissue-specific and developmentally-specific marker molecules can be analyzed to determine their correlation to characteristics such as developmental competence or incompetence, or to the ability to differentiate along a given lineage, using techniques well known to the skilled artisan. For example, Pearson correlation, as described in Golub et al., 1999, Science 286: 531-7; hierarchical clustering as described in (Iyer et. al., '99); and Euclidian distance analysis as described in Golub et al., 1999, Science 286: 531-7 can be used to predict which marker molecules are most closely related to a given characteristic.

Preferably, neighbor analysis as described in Golub et al., 1999, Science 286: 531-7, can be used to identify an idealized expression pattern that predicts a given characteristic. In this method, differences between classes relative to the standard deviation with each class are considered. Each gene or EST is represented by an expression vector e_{g}=(e_{g1}, e_{g2}, e_{g3}, . . . , e_{gs}), where e_{gi} denotes the log expression level of gene g in the i^{th} sample, for a total of s samples on two classes. The statistic P(g,c)=[μ₁(g)−μ₂(g)]/[σ₁(g)+σ₂(g)], where μₖ(g) and σₖ(g) denote the mean and standard deviation of the log expression levels of gene g across Sₖ samples in class k relates to the degree of correlation between a gene or EST and a given characteristic. Large values of (P(g,c)) indicate a strong correlation, while low values indicate a weak correlation, while the sign indicates in which class the gene or EST is more strongly expressed.

Finally, the observed correlations are examined by neighbor analysis to determine whether the density of genes correlated with a given characteristic is greater than would be predicted by chance.

E. Identifying Differentially Expressed Protein Markers

Tissue-specific and developmentally-specific nucleic acid molecules can be identified and characterized by various protein biochemistry techniques known to the skilled artisan, including immunoblotting, competitive or noncompetitive immunoassay, and immunoprecipitation, and by various nonimmunological methods such as analytical centrifugation, amino acid analysis, sequencing, 1- and 2-dimensional electrophoresis (including both native and denaturing conditions such as SDS-PAGE), chromatography, peptide mapping, nuclear magnetic resonance, electron crystallography, and X-ray crystallography. See generally, Deutscher, ed., 1990, Methods in Enzymology, Volume 182, Academic Press, San Diego, Calif. Particularly preferred methods, comprised under the general heading of “proteomics,” and including 2-dimensional electrophoresis coupled with mass spectroscopy, particularly MALDI-TOF mass spectroscopy, can provide insights into gene expression beyond the mRNA level, including posttranslational modifications that cannot be predicted based solely on a nucleic acid sequence. See, e.g., VanBogelen et al., 1999, Electrophoresis 20: 2149-59; Hatzimanikatis et al., 1999, Biotech. Prog. 15: 312-8; and Blackstock and Weir, 1999, Trends Biotech. 17: 121-7

II. Nuclear Transfer Procedures

Nuclear transfer procedures, i.e., methods in which a full complement of nuclear DNA is introduced from one cell into a second, enucleated, cell are well known to a person of ordinary skill in the art. See, U.S. Pat. No. 4,994,384 to Prather et al., entitled “Multiplying Bovine Embryos,” issued on Feb. 19, 1991; U.S. Pat. No. 5,057,420 to Massey, entitled “Bovine Nuclear Transplantation,” issued on Oct. 15, 1991; U.S. Pat. No. 5,994,619, issued on Nov. 30, 1999 to Stice et al., entitled “Production of Chimeric Bovine or Porcine Animals Using Cultured Inner Cell Mass Cells; U.K. Patents Nos. GB 2,318,578 GB 2,331,751, issued on Jan. 19, 2000 to Campbell et al. and Wilmut et al., respectively, entitled “Quiescent Cell Populations For Nuclear Transfer”; and U.S. Pat. No. 6,011,197 to Strelchenko et al., entitled “Method of Cloning Bovines Using Reprogrammed Non-Embryonic Bovine Cells,” issued on Jan. 4, 2000, each of which are hereby incorporated by reference in its entirety including all figures, tables and drawings.

A. Nuclear Donors

Nuclear donor material used to establish a mammalian nuclear transfer embryo can be obtained from a variety of cell types, including cultured and non-cultured cells isolated from an embryo arising from the union of two gametes in vitro or in vivo; cultured and non-cultured pluripotent cells, such as embryonic stem cells (ES cells) arising from cultured embryonic cells (e.g., pre-blastocyst cells and inner cell mass cells); cultured and non-cultured cells arising from inner cell mass cells isolated from embryos; cultured and non-cultured pre-blastocyst cells; cultured and non-cultured fetal cells; cultured and non-cultured primordial germ cells; cultured and non-cultured germ cells (e.g., embryonic germ cells); cultured and non-cultured somatic cells isolated from an animal or fetus; cultured and non-cultured cumulus cells; cultured and non-cultured amniotic cells; cultured and non-cultured fetal fibroblast cells; cultured and non-cultured genital ridge cells; cultured and non-cultured differentiated cells; cultured and non-cultured cells in a synchronous population; cultured and non-cultured cells in an asynchronous population; cultured and non-cultured serum-starved cells; cultured and non-cultured permanent cells; and cultured and non-cultured totipotent cells.

Particularly preferred mammalian nuclear donor cells are canids, felids, murids, leporids, mustelids, ursids, human and non-human primates, ungulates, ovids, suids, equids, bovids, caprids, and cervids. While pluripotent nuclear donor cells can typically give rise to the cloned embryos of the invention, a totipotent nuclear donor cell is generally preferable. For nuclear transfer techniques, a donor cell may be separated from a growing cell mass, isolated from a primary cell culture, and/or isolated from a cell line. An entire cell may be placed in the perivitelline space of a recipient oocyte or may be directly injected into a recipient oocyte by aspirating the nuclear donor into a needle or a Piezo drill, placing the needle/drill tip into a recipient oocyte, releasing the nuclear donor and removing the needle without significantly disrupting the plasma membrane of the oocyte. Also, a nucleus (e.g., a karyoplast) may be isolated from a nuclear donor and placed into the perivitelline space of a recipient oocyte or may be injected directly into a recipient oocyte, for example.

A variety of methods for culturing nuclear donor cells exist in the art. See, e.g., Culture of Animal Cells: a manual of basic techniques (3rd edition), 1994, Freshney (ed.), Wiley-Liss, Inc.; Cells: a laboratory manual (vol. 1), 1998), Spector, Goldman, Leinwand (eds.), Cold Spring Harbor Laboratory Press; and Animal Cells: culture and media, 1994, Darling & Morgan, John Wiley and Sons, Ltd., each of which is incorporated herein by reference in its entirety including all figures, tables, and drawings.

B. Transgenic Nuclear Donor Cells

Materials and methods readily available to a person of ordinary skill in the art can be utilized to convert the nuclear donor cells of the invention (e.g., amniotic cells and follicular cells) into transgenic cells. Once nuclear DNA is modified in a nuclear donor cell, embryos, fetuses, and animals arising from these cells can also comprise the modified nuclear DNA. Hence, materials and methods readily available to a person of ordinary skill in the art can be applied to nuclear donor cells to produce transgenic cloned and chimeric animals. See, e.g., EPO 264 166, entitled “Transgenic Animals Secreting Desired Proteins Into Milk”; WO 94/19935, entitled “Isolation of Components of Interest From Milk”; WO 93/22432, entitled “Method for Identifying Transgenic Pre-implantation Embryos”; WO 95/17085, entitled “Transgenic Production of Antibodies in Milk;” Hammer et al., 1985, Nature 315: 680-685; Miller et al., 1986, J. Endocrinology 120: 481-488; Williams et al., 1992, J. Ani. Sci. 70: 2207-2111; Piedrahita et al., 1998, Biol. Reprod. 58: 1321-1329; Piedrahita et al., 1997, J. Reprod. Fert. (suppl.) 52: 245-254; and Nottle et al, 1997, J. Reprod. Fert. (suppl.) 52: 245-254, each of which is incorporated herein by reference in its entirety including all figures, drawings and tables.

Methods for generating transgenic cells typically include (1) assembling a suitable DNA construct useful for inserting a specific DNA sequence into nuclear DNA of a cell; (2) transfecting the DNA sequence into cells; (3) allowing random insertion and/or homologous recombination to occur. A modification resulting from such a process may include insertion of a suitable DNA construct(s) into a target genome; deletion of DNA from a target genome; and/or mutation of a target genome.

DNA constructs can comprise a gene of interest as well as a variety of elements including regulatory promoters, insulators, enhancers, and repressors as well as elements for ribosomal binding to RNA transcribed from a DNA construct. DNA constructs can also encode ribozymes and anti-sense DNA and/or RNA. Moreover, DNA constructs can comprise a selection element, such as a gene for drug selection of transformants. These examples are well known to a person of ordinary skill in the art and are not meant to be limiting.

Due to effective recombinant DNA techniques available in conjunction with DNA sequences for regulatory elements and genes readily available in data bases and the commercial sector, a person of ordinary skill in the art can readily generate a DNA construct appropriate for establishing transgenic cells using materials and methods described herein. For example, transfection techniques are well known to a person of ordinary skill in the art and materials and methods for carrying out transfection of DNA constructs into cells are commercially available. For example, materials that can be used to transfect cells with DNA constructs are lipophillic compounds, such as Lipofectin™, Superfect™, LipoTAXI™, and CLONfectin™. Particular lipophillic compounds can be induced to form liposomes for mediating transfection of the DNA construct into the cells. In addition, cationic based transfection agents that are known in the art can be utilized to transfect cells with nucleic acid molecules (e.g., calcium phosphate precipitation, DEAE-dextran, polybrene, polyamine). Other techniques are known in the art that use protein-based or amphipathic polyamines as transfection reagents. Also, electroporation techniques known in the art can be utilized to translocate nucleic acid molecules into cells. Furthermore, particle bombardment techniques known in the art can be utilized to introduce exogenous DNA into cells. Target sequences from a DNA construct can be inserted into specific regions of nuclear DNA by rational design of a DNA construct. Such design techniques and methods are well known to a person of ordinary skill in the art. See, U.S. Pat. No. 5,633,067, “Method of Producing a Transgenic Bovine or Transgenic Bovine Embryo,” DeBoer et al., issued May 27, 1997; U.S. Pat. No. 5,612,205, “Homologous Recombination in Mammalian Cells,” Kay et al., issued Mar. 18, 1997; and PCT publication WO 93/22432, “Method for Identifying Transgenic Pre-Implantation Embryos,” each of which is incorporated herein by reference in its entirety, including all figures, drawings, and tables. Once a desired DNA sequence is inserted into the nuclear DNA of a cell, the location of an insertion region as well as the frequency with which the desired DNA sequence has inserted into the nuclear genome can be identified by methods well known to those skilled in the art.

Once a transgene or transgenes are inserted into nuclear DNA of a nuclear donor cell, that cell can be used for cloning a transgenic animal. A description of embodiments related to transgenic animals are described in further detail hereafter.

i. Diseases and Parasites

Desired DNA sequences can be inserted into nuclear DNA of a cell to enhance the resistance of a cloned transgenic animal to particular parasites, diseases, and infectious agents. Examples of parasites include worms, flies, ticks, and fleas. Examples of infectious agents include bacteria, fingi, and viruses. Examples of diseases include Atrophic rhinitis, Cholera, Leptospirosis, Pseudorabies, Pasturellosis, and Brucellosis. These examples are not limiting and the invention relates to any disease or parasite or infectious agent known in the art. See, e.g., Hagan & Bruners Infectious Diesases of Domestic Animals (7th edition), Gillespie & Timoney, copyright 1981, Cornell University Press, Ithaca NY.

A transgene can confer resistance to a particular parasite or disease by completely abrogating or partially alleviating symptoms of the disease or parasitic condition, or by producing a protein which controls the parasite or disease.

ii. Elements of DNA Constructs and Production of DNA Constructs

A wide variety of transcriptional and translational regulatory sequences may be inserted into nuclear DNA of a nuclear donor cell. Transcriptional and translational regulatory signals may be derived from viral sources, such as adenovirus, bovine papilloma virus, cytomegalovirus, simian virus, or the like, whereas the regulatory signals can be associated with a particular gene sequence having a potential for high levels of expression. Additionally, promoters from mammalian expression products, such as actin, casein, alpha-lactalbumin, uroplakin, collagen, myosin, and the like, may be employed. Transcriptional regulatory signals may be selected which allow for repression or activation, so that expression of a gene product can be modulated. Of interest are regulatory signals which can be repressed or initiated by external factors such as chemicals or drugs. These examples are not limiting and the invention relates to any regulatory elements. Other examples of regulatory elements are described herein.

iii. Examples of Preferred Recombinant Products

A variety of proteins and polypeptides can be encoded by a gene harbored within a DNA construct suitable for creating transgenic cells. Those proteins or polypeptides include hormones, growth factors, enzymes, clotting factors, apolipoproteins, receptors, drugs, pharmaceuticals, bioceuticals, nutraceuticals, oncogenes, tumor antigens, tumor suppressors, cytokines, viral antigens, parasitic antigens, bacterial antigens and chemically synthesized polymers and polymers biosynthesized and/or modified by chemical, cellular and/or enzymatic processes. Specific examples of these compounds include proinsulin, insulin, growth hormone, androgen receptors, insulin-like growth factor I, insulin-like growth factor II, insulin growth factor binding proteins, epidermal growth factor, TGF-α, TGF-β, dermal growth factor, platelet derived growth factor (PDGF), angiogenesis factors (e.g., acidic fibroblast growth factor, basic fibroblast growth factor, and angiogenin), angiogenesis inhibitors (e.g., endostatin and angiostatin), matrix proteins (Type IV collagen, Type VII collagen, laminin), oncogenes (ras, fos, myc, erb, src, sis, jun), E6 or E7 transforming sequence, p53 protein, cytokine receptor, IL-1, IL-6, IL-8, IL-2, α, β, or γ IFN, GMCSF, GCSF, viral capsid protein, and proteins from viral, bacterial and parasitic organisms. Other specific proteins or polypeptides which can be expressed include: phenylalanine hydroxylase, α-1-antitrypsin, cholesterol-7β-hydroxylase, truncated apolipoprotein B, lipoprotein lipase, apolipoprotein E, apolipo-protein A1, LDL receptor, scavenger receptor for oxidized lipoproteins, molecular variants of each, VEGF, and combinations thereof. Other examples are antibodies (monoclonal or polyclonal), antibody fragments, clotting factors, apolipoproteins, drugs, tumor antigens, viral antigens, parasitic antigens, monoclonal antibodies, and bacterial antigens. One skilled in the art readily appreciates that these proteins belong to a wide variety of classes of proteins, and that other proteins within these classes or outside of these classes can also be used. These are only examples and are not meant to be limiting in any way.

It should also be noted that the genetic material which is incorporated into the cells from DNA constructs includes (1) nucleic acid sequences not normally present in target cells; (2) nucleic acid molecules which are normally present in target cells but not expressed at physiological significant levels; (3) nucleic acid sequences normally present in target cells and normally expressed at physiological desired levels; (4) other nucleic acid sequences which can be modified for expression in target cells; and (5) any combination of the above.

In addition, DNA constructs may become incorporated into nuclear DNA of cells, where incorporated DNA can be transcribed into ribonucleic acid molecules that can cleave other RNA molecules at specific regions. Ribonucleic acid molecules which can cleave RNA molecules are referred to in the art as ribozymes. Ribozymes are themselves RNA molecules. Ribozymes can bind to discrete regions on a RNA molecule, and then specifically cleave a region within that binding region or adjacent to the binding region. Ribozyme techniques can thereby decrease the amount of polypeptide translated from formerly intact message RNA molecules.

Furthermore, DNA constructs can be incorporated into nuclear DNA of cells and when transcribed produce RNA that can bind to both specific RNA or DNA sequences. Nucleic acid sequences can be utilized in anti-sense techniques, where nucleic acids bind to a message (mRNA) in order to block translation. Anti-sense techniques can thereby block or partially block the synthesis of particular polypeptides in cells.

C. Recipient Cells

A recipient cell is a cell into which the nuclear donor is inserted. Preferably, the recipient cell is enucleated, i.e., the recipient cell nucleus chromosomal material is removed or inactivated. A recipient cell is preferably an oocyte with a portion of its ooplasm removed, where the removed ooplasm comprises the oocyte nucleus genetic material. Enucleation techniques are well known to a person of ordinary skill in the art, as described hereafter. Other recipient cells, e.g., a two cell enucleated embryo, are known to the ordinarily skilled artisan. A recipient cell can also be rendered “functionally enucleated,” for example by ultraviolet irradiation. See, e.g., Bradshaw et al. (1995), Molecular Reproduction and Development 41:503-12.

i. Isolation of Oocytes

Oocytes can be isolated from oviducts and/or ovaries of live animals by oviductal recovery procedures or transvaginal oocyte recovery procedures well known in the art. Furthermore, oocytes can be isolated from deceased animals. For example, ovaries can be obtained from abattoirs and oocytes can be aspirated from these ovaries. Oocytes can also be isolated from ovaries of a recently sacrificed animal or when an ovary has been frozen and/or thawed. Oocytes may be isolated from ovarian follicles at any stage of development, including primordial follicles, primary follicles, secondary follicles, growing follicles, vesicular follicles, maturing follicles, mature follicles, and graafian follicles. Moreover, oocytes can be isolated from follicles which are obtained from animals, and which are grown and matured in culture. Materials and methods for isolating oocytes from various developmental stages of ovarian follicles are known to those skilled in the art. See, e.g., Laboratory Production of Cattle Embryos, 1994, Ian Gordon, CAB International; Anatomy and Physiology of Farm Animals (5th ed.), 1992, R. D. Frandson and T. L. Spurgeon, Lea & Febiger, each of which is incorporated herein by reference in its entirety including all figures, drawings, and tables.

In preferred embodiments, the recipient oocyte is a mammalian oocyte. Particularly preferred are canid, felid, murid, leporid, mustelid, human and non-human primate, ungulate, ovid, suid, equid, bovid, caprid, and cervid recipient oocytes. A nuclear donor cell and a recipient oocyte may be isolated from an animal of the same species or different species. For example, a porcine cumulus cell can be inserted into a porcine enucleated oocyte. Alternatively, a wild boar cumulus cell can be inserted into a domesticated porcine oocyte. Any nuclear donor/recipient oocyte combinations are envisioned by the invention. Preferably a nuclear donor and recipient oocyte are isolated from the same species. Xenospecific NT techniques can be utilized to produce cloned animals that are endangered or extinct.

Oocytes can be activated by electrical and/or non-electrical means before, during, and/or after a nuclear donor is introduced to recipient oocyte. For example, an oocyte can be placed in a medium containing one or more components suitable for non-electrical activation prior to fusion with a nuclear donor. Also, a cybrid can be placed in a medium containing one or more components suitable for non-electrical activation. Activation processes are discussed in greater detail hereafter.

ii. Oocyte Maturation

Oocytes and cumulus cell/oocyte complexes can be matured in vivo, and more preferably, can be matured in an in vitro environment. The length of time oocytes is matured can vary, depending upon species. In preferred embodiments, oocytes can be matured for (1) greater than about 10 hours; (2) greater than about 20 hours; (3) greater than about 24 hours; (4) greater than about 30 hours; (5) greater than about 40 hours; (6) greater than about 50 hours; (7) greater than about 60 hours (8) greater than about 72 hours; (9) greater than about 80 hours; (10) greater than about 90 hours; and (11) greater than about 100 hours. The term “about” with respect to oocyte maturation refers to plus or minus 5 hours.

A variety of media well known to a person of ordinary skill in the art can be used for maturing oocytes in vitro. See, e.g., (i) Alm & Hinrichs, 1996, J. Reprod. Fert. 107: 215-220 and Alm & Torner, 1994, Theriogenology 42: 345-349 for equine oocytes; (ii); Ledda et al., 1997, Journal of Reproduction and Fertility 109:73-78; Byrd et al., 1997, Theriogenology 47: 857-864; Wilmut et al., 1997, Nature 385: 810-813; and LeGal, 1996, Theriogenology 45: 1177-1 for caprine and ovine oocytes; (iii); Lorenzo et al., 1996, Journal of Reproduction and Fertility 107:109-117 and Jelinkova et al., 1994, Molecular Reproduction and Development 37:210-215 for leporidine oocytes; (iv) Nickson et al., 1993, J. Reprod. Fert. (Suppl. 47): 231-240; Yamada et al., 1993, J. Reprod. Fert. (Suppl. 47): 227-229; and Mahi & Yanagimachi, 1976, Journal of Experimental Zoology 196; 189-196 for canine oocytes; (v) Fukui et al., 1991, Theriogenology 35: 499-512 and Pollard et al., 1995, Theriogenology 43: 301 for cervidine oocytes; and (vi) Del Campo et al., 1995, Theriogenology 43: 21-30 and Del Campo et al., 1994, Theriogenology 41: 187 for camelid oocytes. One example of such a medium suitable for maturing oocytes in vitro is depicted in an exemplary embodiment described herein. Oocytes can be successfully matured in such a medium within an environment comprising 5% CO₂ at 39° C. Oocytes may be cryopreserved and then thawed before placing the oocytes in maturation medium. Cryopreservation procedures for cells and embryos are well known in the art as discussed herein.

Components of an oocyte maturation medium can include molecules that arrest oocyte maturation. Examples of such components are 6-dimethylaminopurine (DMAP) and isobutylmethylxanthine (IBMX). IBMX has been reported to reversibly arrest oocytes, but the efficiencies of arrest maintenance are quite low. See, e.g., Rose-Hellkant and Bavister, 1996, Mol. Reprod. Develop. 44: 241-249. However, oocytes may be arrested at the germinal vesicle stage with a relatively high efficiency by incubating oocytes at 31° C. in an effective concentration of IBMX. Preferably, oocytes are incubated the entire time that oocytes are collected. Concentrations of IBMX suitable for oocyte maturation are 0.01 mM to 20 mM IBMX, preferably 0.05 mM to 10 mM IBMX, and more preferably about 0.1 mM IBMX to about 0.5 mM IBMX, and most preferably 0.1 mM IBMX to 0.5 mM IBMX. The exemplary oocyte maturation procedures are not meant to be limiting and the invention relates to any oocyte maturation procedure known to a person of ordinary skill in the art.

D. Nuclear Transfer

A nuclear donor can be translocated into a nuclear acceptor, preferably an oocyte, most preferably an enucleated oocyte, using a variety of materials and methods that are well known to a person of ordinary skill in the art. In one example, a nuclear donor may be directly injected into a recipient oocyte. This direct injection can be accomplished by gently pulling a nuclear donor into a needle, piercing a recipient oocyte with that needle, releasing the nuclear donor into the oocyte, and removing the needle from the oocyte without significantly disrupting its membrane. Appropriate needles can be fashioned from glass capillary tubes, as defined in the art and specifically by publications incorporated herein by reference.

In another example, at least a portion of plasma membrane from a nuclear donor and recipient oocyte can be fused together by utilizing techniques well known to a person of ordinary skill in the art. See, Willadsen, 1986, Nature 320:63-65, hereby incorporated herein by reference in its entirety including all figures, tables, and drawings. Typically, lipid membranes can be fused together by electrical and chemical means, as defined previously and in other publications incorporated herein by reference.

Examples of non-electrical means of cell fusion involve incubating cybrids in solutions comprising polyethylene glycol (PEG), and/or Sendai virus. PEG molecules of a wide range of molecular weight can be utilized for cell fusion.

Processes for fusion that are not explicitly discussed herein can be determined without undue experimentation. For example, modifications to cell fusion techniques can be monitored for their efficiency by viewing the degree of cell fusion under a microscope. The resulting cybrid can then be cloned and identified as totipotent by the methods described below for identifying totipotent cells, which can include tests for selectable markers and/or tests for developing an animal.

E. Activation

Examples of electrical processes for activation are well known in the art. Although electrical pulses are sometimes sufficient for stimulating cell activation, other non-electrical means for activation are useful and are often necessary for proper activation of a cell. Electrical and non-electrical activation may be used separately, or may be used together for activating a cell. Chemical materials and methods useful for non-electrical activation are described below in other preferred embodiments of the invention. When two or more chemical components are introduced to a cell for activating the cell, the components can be added simultaneously or individually in steps.

Examples of components that are useful for non-electrical activation include ethanol; inositol trisphosphate (IP3); divalent ions (e.g., addition of Ca2+ and/or Sr2+); microfilament inhibitors (e.g., cytochalasin B); ionophores for divalent ions (e.g., the Ca2+ ionophore ionomycin); protein kinase inhibitors (e.g., 6-dimethylaminopurine (DMAP)); protein synthesis inhibitors (e.g., cyclohexamide); phorbol esters such as phorbol 12-myristate 13-acetate (PMA); and thapsigargin. It is also known that temperature change and mechanical techniques are also useful for non-electrical activation. The invention includes any activation techniques known in the art. See, e.g., U.S. Pat. No. 5,496,720, entitled “Parthenogenic Oocyte Activation,” issued on Mar. 5, 1996, Susko-Parrish et al., and Wakayama et al., 1998, Nature 394: 369-374, each of which is incorporated herein by reference in its entirety, including all figures, tables, and drawings.

Examples of preferred protein kinase inhibitors are protein kinase A, G, and C inhibitors such as 6-dimethylaminopurine (DMAP), staurosporin, 2-aminopurine, sphingosine. Tyrosine kinase inhibitors may also be utilized to activate cells.

Preferred methods for activating cells are depicted in exemplary embodiments described herein. Activation materials and methods that are not explicitly discussed herein can be identified by modifying specified conditions defined in exemplary protocols described hereafter and in U.S. Pat. No. 5,496,720.

Activation efficiency and totipotency that result from any modifications of activation procedures can be identified by methods described herein. Methods for identifying totipotent embryos can include one or more tests, such as (a) identifying specific markers for totipotent cells in embryos, and (b) by determining whether embryos are totipotent by allowing them to develop into an animal. Therefore, the invention relates to any modifications to activation procedures described herein even though these modifications may not be explicitly stated herein.

F. Manipulation of Embryos, Fetuses, and Animals Resulting from Nuclear Transfer

An embryo resulting from a NT process can be manipulated in a variety of manners. The invention relates to cloned embryos, fetuses, and animals that arise from at least one NT. Two or more NT procedures may be performed to enhance nuclear transfer efficiency of totipotent embryo, fetus, and animal production and/or placental development. Incorporating two or more NT cycles into methods for cloned embryos, fetuses, and animals can provide further advantages. For example, incorporating multiple NT procedures provides a method for multiplying the number of cloned embryos, fetuses, and animals. Moreover, gene targeting methods require that both copies of a given gene in a diploid cell be targeted in order to knock out or replace the gene. Such methods may require two or more NT procedures in order to efficiently target the gene. The skilled artisan will understand that the methods required for such manipulations will vary, depending on the species of interest.

When multiple NT procedures are utilized for formation of a cloned embryo, fetus, or animal, oocytes that have been matured for any period of time can be utilized as recipients in the first, second or subsequent NT procedures. For example, if a first NT and then a second NT are performed, the first NT can utilize an oocyte that has been matured for about 53 hours as a recipient and the second NT may utilize an oocyte that has been matured for less than about 53 hours as a recipient. Alternatively, the first NT may utilize an oocyte that has been matured for about 53 hours as a recipient and the second NT may utilize an oocyte that has been matured for greater than about 53 hours as a recipient for a two-cycle NT regime. In addition, both NT cycles may utilize oocytes that have been matured for about 53 hours as recipients, both NT cycles may utilize oocytes that have been matured for less than about 53 hours as recipients, and both NT cycles may utilize oocytes that have been matured for greater than about 53 hours as recipients in a two-cycle NT regime.

For NT techniques that incorporate two or more NT cycles, one or more of the NT cycles may be preceded, followed, and/or carried out simultaneously with an activation step. As defined previously herein, an activation step may be accomplished by electrical and/or non-electrical means as defined herein. An activation step may also be carried out at the same time as a NT cycle (e.g., simultaneously with the NT cycle) and/or an activation step may be carried out prior to a NT cycle. Cloned embryos resulting from a NT cycle can be (1) disaggregated or (2) allowed to develop further.

If embryos are disaggregated, the disaggregated embryonic derived cells can be utilized to establish cultured cells. Any type of embryonic cell can be utilized to establish cultured cells. These cultured cells are sometimes referred to as embryonic stem cells or embryonic stem-like cells in the scientific literature. Embryonic stem cells can be derived from early embryos, morulae, and blastocyst stage embryos. Multiple methods are known to a person of ordinary skill in the art for producing cultured embryonic cells. These methods are enumerated in specific references previously incorporated by reference herein.

If embryos are allowed to develop into a fetus in utero, or a live-born animal, cells isolated from that fetus or animal can be utilized to establish cultured cells. In preferred embodiments, primordial germ cells, genital ridge cells, and fetal fibroblast cells can be isolated from a fetus. Similarly, a variety of somatic cells or germ cells can be isolated form a live-born animal. Such cultured cells can be established by utilizing culture methods well known to a person of ordinary skill in the art. Such methods are enumerated in publications previously incorporated herein by reference and are discussed herein.

Cloned embryos resulting from NT can also be manipulated by cryopreserving and/or thawing such embryos. See, e.g., Nagashima et al., 1989, Japanese J. Anim. Reprod. 35: 130-134 and Feng et al., 1991, Theriogenology 35: 199, each of which is incorporated herein by reference in its entirety including all tables, figures, and drawings. Other embryo manipulation methods include in vitro culture processes; performing embryo transfer into a maternal recipient; disaggregating blastomeres for NT processes; disaggregating blastomeres or inner cell mass cells for establishing cell lines for use in NT procedures; embryo splitting procedures; embryo aggregating procedures; embryo sexing procedures; and embryo biopsying procedures. Exemplary manipulation procedures are not meant to be limiting and the invention relates to any embryo manipulation procedure known to a person of ordinary skill in the art.

G. Development of Cloned Embryos

i. Culture of Embryos In Vitro

Cloning procedures discussed herein provide an advantage of culturing cells and embryos in vitro prior to implantation into a recipient female. Methods for culturing embryos in vitro are described in detail herein. In addition, exemplary embodiments for media suitable for culturing cloned embryos in vitro are described hereafter. Feeder cell layers may or may not be utilized for culturing cloned embryos in vitro. Feeder cells are described previously and in exemplary embodiments hereafter.

ii. Development of Embryos In Vivo

Cloned embryos can be cultured in an artificial or natural uterine environment after NT procedures. Moreover, cloned embryos can be cultured in vivo prior to, subsequent to, or in the absence of culture of the embryo in vitro. Examples of artificial development environments are being developed and some are known to those skilled in the art. Components of the artificial environment can be modified, for example, by altering the amount of a component or components and by monitoring the growth rate of an embryo.

Methods for implanting embryos into the uterus of an animal are also well known in the art, as discussed previously. Preferably, developmental stage of the embryo(s) is correlated with the estrus cycle of an animal.

Embryos from one species can be placed into a uterine environment in an animal from another species. For example it has been shown in the art that bovine embryos can develop in oviducts of sheep. Stice & Keefer, 1993, “Multiple generational bovine embryo cloning,” Biology of Reproduction 48: 715-719. The invention relates to any combination of an embryo in any homospecific or xenospecific uterine environment. A xenospecific in utero development regime can allow for efficient production of cloned animals of an endangered species. For example, a wild boar embryo can develop in the uterus of a domestic porcine sow.

Once an embryo is placed into the uterus of a recipient female, the embryo can develop to term. Alternatively, an embryo can be allowed to develop in the uterus and then can be removed at a chosen time. Surgical methods are well known in the art for removing fetuses from uteri before parturition.

III. Materials and Methods for Oocyte Maturation, Oocyte Enucleation, Cell Activation, In Vitro Embryo Development, and Other Processes

Where descriptions of oocyte maturation, oocyte enucleation, cell activation, in vitro embryo development, and other processes are described herein in relation to mammals in general, the following references provide additional descriptions of such process for specific mammals. The following references are provided to aid the reader in understanding the invention and are not admitted to describe or constitute prior art to the present invention. With regard to suids, researchers have reported materials and methods for oocyte maturation, oocyte enucleation, cell activation, in vitro embryo development, and other processes. See, e.g., Grocholová et al., 1997, J. Exp. Zoology 277: 49-56; Schoenbeck et al., 1993, Theriogenology 40: 257-266; Prather et al., 1989, Biology of Reproduction 41: 414-418; Prather et al., 1991, Molecular Reproduction and Development 28: 405-409; Jolliff & Prather, 1997, Biol. Reprod. 56: 544-548; Mattioli et al., 1991, Molecular Reproduction and Development 30: 109-125; Terlouw et al., 1992, Theriogenology 37: 309; Prochazka et al., 1992, J. Reprod. Fert. 96: 725-734; Funahashi et al., 1993, Molecular Reproduction and Development 36: 361-367; Prather et al., Bio. Rep. Vol. 50 Sup 1: 282; Nussbaum et al., 1995, Molecular Reproduction and Development 41: 70-75; Funahashi et al., 1995, Zygote 3: 273-281; Wang et al., 1997, Biology of Reproduction 56: 1376-1382; Piedrahita et al., 1989, Biology of Reproduction 58: 1321-1329; Machaty et al., 1997, Biology of Reproduction 57: 85-91; and Machaty et al., 1995, Biology of Reproduction 52: 753-758.

With regard to bovids, researchers have reported materials and methods for oocyte maturation, oocyte enucleation, cell activation, in vitro embryo development, and other processes. See, e.g., U.S. Pat. Nos. 5,453,357 and 5,670,372, entitled “Pluripotent Embryonic Stem Cells and Methods of Making Same,” Hogan; Sims & First, 1993, Theriogenology 39:313; Keefer et al., 1994, Mol. Reprod. Dev. 38:264-268; U.S. Pat. No. 4,994,384, “Multiplying Bovine Embryos,” Prather et al.; U.S. Pat. No. 5,057,420, “Bovine Nuclear Transplantation,” Massey & Willadsen; Delhaise et al., 1995, Reprod. Fert. Develop. 7:1217-1219; Lavoir 1994, J. Reprod. Dev. 37:413-424; PCT application WO 95/10599 entitled “Embryonic Stem Cell-Like Cells”; Stice et al., 1996, Biol. Reprod. 54: 100-110; Strelchenko, 1996, Theriogenology 45: 130-141; WO 97/37009, entitled “Cultured Inner Cell Mass Cell-Lines Derived from Ungulate Embryos,” Stice and Golueke, published Oct. 9, 1997; U.S. Pat. No. 5,213,979, entitled “In vitro Culture of Bovine Embryos,” First et al., May 25, 1993; U.S. Pat. No. 5,096,822, entitled “Bovine Embryo Medium,” Rosenkrans, Jr. et al., Mar. 17, 1992; Seidel and Elsden, 1997, Embryo Transfer in Dairy Cattle, W. D. Hoard & Sons, Co., Hoards Dairyman; Stice & Keefer, 1993, “Multiple generational bovine embryo cloning,” Biology of Reproduction 48: 715-719; Wagoner et al., 1996, “Functional enucleation of bovine oocytes: effects of centrifugation and ultraviolet light,” Theriogenology 46: 279-284; Pieterse et al., 1988, “Aspiration of bovine oocytes during transvaginal ultrasound scanning of the ovaries,” Theriogenology 30: 751-762; Saito et al., 1992, Roux's Arch. Dev. Biol. 201: 134-141; and U.S. Pat. No. 5,496,720, entitled “Parthenogenic Oocyte Activation,” Mar. 5, 1996, Susko-Parrish et al.

With regard to felids, researchers have reported materials and methods for oocyte maturation, oocyte enucleation, cell activation, in vitro embryo development, and other processes. See, e.g., Swanson et al., 1996, Molecular Reprod. Dev. 43: 298-305; Donoghue et al., 1996, J. Reprod. and Fertility 107: 53-58; Goritz et al., 1996, J. Reprod. and Fertility 106: 117-124; Hoffert et al., 1997, Molecular Reprod. Dev. 48: 208-215; Donoghue et al., 1990, Biology of Reprod. 43: 733-744; Wood et al., 1995, J. Reprod. Fertility 104: 315-323; Donoghue et al., 1992, Biology Reprod. 46: 972-980; Johnston et al., 1991, J. Reprod. Fert 92: 377-382; Luvoni et al., 1993, J. Reprod. Fert. Suppl. 47: 203-207; Roth et al., 1997, Biology of Reprod. 57: 165-171; and Jewgenow, 1996, Theriogenology 45: 889-895.

With regard to canids, researchers have reported materials and methods for oocyte maturation, oocyte enucleation, cell activation, in vitro embryo development, and other processes. See, e.g., Nickson et al., 1993, J. Reprod. Fert. (Suppl. 47): 231-240; Yamada et al., 1993, J. Reprod. Fert. (Suppl. 47): 227-229; Mahi &Yanagimachi, 1976, Journal of Experimental Zoology 196; 189-196; Yamada et al., 1992, Biology of Reproduction 46: 853-858; Farstad et al., 1993, Journal of Reproduction and Fertility (Suppl. 47): 219-226; Bolamba et al., 1998, Theriogenology 49: 933-942; Durrant et al., 1998, Theriogenology 49: 917-932; and Hewitt et al., 1998, Theriogenology 49: 1083-1101.

With regard to equids, researchers have reported materials and methods for oocyte maturation, oocyte enucleation, cell activation, in vitro embryo development, and other processes. See, e.g., Alm & Hinrichs, 1996, J. Reprod. Fert. 107: 215-220; Alm & Torner, 1994, Theriogenology 42: 345-349; Hinrichs et al., 1993, Biol. Reprod. 48: 363-370; Hinrichs et al., 1995, J. Reprod. Fert. 104: 149-156; Hinrichs et al., 1995, Biology of Reproduction Monograph 1: 319-324; and Dell'Aquila et al., 1997, Theriogenology 47: 1139-1156.

With regard to ovids and caprids, researchers have reported materials and methods for oocyte maturation, oocyte enucleation, cell activation, in vitro embryo development, and other processes. See, e.g., Willadsen, 1986, Nature 320: 63-66; Ruffing et al., 1993, Biology of Reproduction 48: 889-904; Smith & Wilmut, 1989, Biology of Reproduction 40: 1027-1035; McLaughlin et al., 1991, Theriogenology 35: 240; Campbell et al., 1995, Theriogenology 43: 181; Cambell et al., 1996, Theriogenology 45: 286; Campbell et al., 1996, Nature 380: 64-66; Wilmut et al., 1997, Nature 385: 810-813; Ledda et al., 1997, Journal of Reproduction and Fertility 109:73-78; Byrd et al., 1997, Theriogenology 47: 857-864; Wilmut et al., 1997, Nature 385: 810-813; LeGal, 1996, Theriogenology 45: 1177-1; Pawshe et al., 1996, Theriogenology 46: 971-982; Gall et al., 1993, Molecular Reproduction and Development 36: 500-506; Walker et al., 1996, Biology of Reproduction 55: 703-708; and Gardner et al., 1994, Biology of Reproduction 50: 390-400.

With regard to murids, researchers have reported materials and methods for oocyte maturation, oocyte enucleation, cell activation, in vitro embryo development, and other processes. See, e.g., Downs & Mastropolo, 1997, Mol. Reprod. Dev. 46: 551-566; Kim & Schuetz, 1991, Cell Tissue Res. 265: 105-112; Downs, 1995, Dev. Biol. 167: 502-512; Kito & Bavister; 1997, J. Reprod. Fert. 110: 35-46; Zhang & Rutledge, 1991, Mol. Reprod. Dev. 28: 292-296; Bos-Mickich & Whitingharn, 1995, Mol. Reprod. Devel. 42: 254-260; Cuthbertson, 1983, J. Exp. Zool. 226: 311-314; Shaw & Trounson, 1989, Gamete Res. 24: 269-279; Sakkas & Trounson, 1991, Reprod. Fert. Dev. 3: 99-108; Kito & Bavister, 1997, J. Reprod. Fert. 110: 35-46; Bavister, 1995, Human Reprod. Update 1: 91-148; Erbach et al., 1994, Biol. Reprod. 50: 1027-1033; and Ho et al., 1995, Mol. Reprod. Dev. 41: 232-238.

With regard to leporids, researchers have reported materials and methods for oocyte maturation, oocyte enucleation, cell activation, in vitro embryo development, and other processes. See, e.g., Kanka et al., 199, Molecular Reproduction and Development 43: 135-144; Lui et al., 1996, Molecular Reproduction and Development 45: 157-162; Du et al., 1995, Journal of Reproduction and Fertility 104: 219-223; Farrell & Foote, 1995, Journal of Reproduction and Fertility 103: 127-130; Sofikitis et al., 1996, Fertility and Sterility 65: 176-185; Adenot et al., 1997, Molecular Reproduction and Development 46: 325-336; Lorenzo et al., 1996, Journal of Reproduction and Fertility 107:109-117; and Jelinkova et al., 1994, Molecular Reproduction and Development 37:210-215.

With regard to mustelids, researchers have reported materials and methods for oocyte maturation, oocyte enucleation, cell activation, in vitro embryo development, and other processes. See, e.g., Johnston et al., 1994, Journal of Experimental Zoology 269: 53-61; Polejaeva et al., 1997, Journal of Reproduction and Fertility 109: 229-236; and Moreau et al., 1995, Biology of Reproduction 53: 511-518.

With regard to cervids, researchers have reported materials and methods for oocyte maturation, oocyte enucleation, cell activation, in vitro embryo development, and other processes. See, e.g., Berg et al., 1995, Theriogenology 44: 247-254; Berg et al., 1994, Theriogenology 41: 160; Fukui et al., 1991, Theriogenology 35: 499-512; and Pollard et al., 1995, Theriogenology 43: 301.

With regard to camelids, researchers have reported materials and methods for oocyte maturation, oocyte enucleation, cell activation, in vitro embryo development, and other processes. See, e.g., Del Campo et al., 1995, Theriogenology 43: 21-30; Del Campo et al., 1994, Theriogenology 41: 187; McKinnon et al., 1994, Theriogenology 41: 145-150; Wiepz & Chapman, 1985, Theriogenology 24: 251-257; and Del Campo et al., 1994, Theriogenology 41: 1219-1229.

With regard to non-human primates, researchers have reported materials and methods for oocyte maturation, oocyte enucleation, cell activation, in vitro embryo development, and other processes. See, e.g., Edward, 1965, Nature (Lond) 208: 349-351; Morgan et al., 1991, Biol. Reprod. 45: 89-93; Meng et al., 1997, Biol. Reprod. 57: 454-459; We et al., 1996, Biol. Reprod. 55: 260-270; Bavister et al., 1983, Biol. Reprod. 28: 983-999; Weston et al., 1996, Mol. Reprod. Dev. 44: 88-92; Enders et al., 1989, Biol. Reprod. 41: 715-727; Wolf et al., 1990, Molec. Reprod. Dev. 27: 261-280; and Zhang et al., 1994, Biol. Reprod. 51: 433-440.

Each reference set forth in this section is incorporated herein by reference in its entirety, including all figures, tables, and drawings.

IV. Cloned Animals

As described previously herein, the invention provides advantages of assessing the genotype and phenotype of an animal before cloning that animal. In preferred embodiments, an animal to be cloned is a mammal. Particularly preferred mammals are canid, felid, murid, leporid, mustelid, ursid, primate, ungulate, ovid, suid, equid, bovid, caprid, and cervid animals. Preferably, a cloned animal has a genome that is substantially similar to that of a nuclear donor cell. More preferably, a cloned animal has a genome that is identical to that of a nuclear donor cell. Differences between a genotype and/or phenotype of the nuclear donor cell and that of the cloned animal can result from natural processes, such as differences in DNA methylation or differences in telomere length for example, and can also result from differences in the intrauterine environment during development. Differences between nuclear donor DNA and that of a cloned animal may also result from the addition of one or more transgenes.

A cloned animal can also be a member of a plurality of animals which share substantially similar, and preferably identical, genomes with a nuclear donor. Differences between a genotype and/or phenotype of each cloned animal and that of other cloned animals descended from a common nuclear donor can result from natural processes, such as differences in DNA methylation or differences in telomere length for example, and can also result from differences in intrauterine environment during development, as described above.

Multiple products can be isolated from a cloned animal. The following discussion of such products is not meant to be limiting and the invention relates to any products which may be isolated or collected from a cloned animal using techniques known to a person of ordinary skill in the art. Products can be any body fluids or organs isolated from the animal, or any products isolated from fluids or organs. In preferred embodiments, products such as meat may be collected from cloned animals. In preferred embodiments, products may be present in mammary tissue of a female transgenic animal, or present in products of mammary tissue such as breast milk (containing one or more recombinant proteins), which may then be collected and subjected to purification techniques. In another example, semen can be collected from a cloned animal and cryopreserved. Semen can also be separated into sex-specific fractions of sperm. See, U.S. Pat. Nos. 5,439,362, 5,346,990, and 5,021,244, entitled “Sex-associated Membrane Proteins and Methods for Increasing the Probability that Offspring Will be of a Desired Sex,” Spaulding, and issued on Aug. 8, 1995, Sep. 13, 1994, and Jun. 4, 1991, respectively, each of which is incorporated herein by reference in its entirety including all figures, drawings, and tables. Methods of collecting semen are well known to a person of ordinary skill in the art, as discussed previously. In another embodiment, the invention relates to determining a phenotype of an animal, which is a neutered animal, and then cloning this animal such that resulting cloned animals are reproductively functional and can be used to produce semen. Other preferred embodiments of the invention relate to such products as xenograft materials, sperm, embryos, oocytes, any type of cells, and offspring harvested from cloned animals of the invention.

Xenograft materials can relate to any cellular material extracted from one organism and placed into another organism. Medical procedures for extracting the cellular material from one organism and grafting it into another organism are well known to a person of ordinary skill in the art. Examples of preferable xenograft cellular materials can be selected from the group consisting of liver, lung, heart, nerve, brain, gallbladder, kidney, skin, bone, small intestine, large intestine, and pancreas cellular material.

As discussed in a previous section, transgenic animals can be generated from methods of the invention by using transgenic techniques well known to those of ordinary skill in the art. Preferably, cloned transgenic animals are produced from such methods. Cloned transgenic animals can be engineered such that they are resistant or partially resistant to diseases and parasites endemic to such animals. Examples of such diseases and parasites are outlined in a preceding section.

Moreover, cloned transgenic animals can be engineered such that they produce a recombinant product. Examples of recombinant products are outlined in a preceding section. Expression of such products can be directed to particular cells or regions within cloned transgenic animals by selectively engineering a suitable promoter element and other regulatory elements to achieve this end.

For example, human recombinant products can be expressed in urine of cattle by operably linking a uroplakin promoter to the DNA sequence encoding a recombinant product. Alternatively, examples are well known to a person of ordinary skill in the art for selectively expressing human recombinant products in milk of an ungulate animal.

Once a recombinant product or recombinant products have been expressed in a particular tissue or fluid of a cloned transgenic animal, suitable tissue or fluid can be collected using methods well known in the art. Recombinant products can be purified from such fluid or tissue by using standard purification techniques well known to a person of ordinary skill in the art.

V. Method for Treating a Disease or Disorder

The present invention also relates to a method for treating a disease or disorder comprising the step of administering to a patient in need of such a treatment one or more molecules identified as inducing or inhibiting developmental competence or as inducing lineage specific development in a cell line.

Toxicity and therapeutic efficacy of substances, or compounds, can be determined by standard pharmaceutical procedures in cell cultures or experimental animals. The dose ratio between toxic and therapeutic effects is the therapeutic index and it can be expressed as the ratio LD50/ED50. Compounds which exhibit large therapeutic indices are preferred. The data obtained from these cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized.

For any compound used in the methods of the invention, the therapeutically effective dose can be estimated initially from cell culture assays. For example, a dose can be formulated in animal models to achieve a circulating plasma concentration range that includes the IC50 as determined in cell culture (e.g., the concentration of the test compound which achieves a half-maximal disruption of the protein complex, or a half-maximal inhibition of the cellular level and/or activity of a complex component). Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., 1975, in “The Pharmacological Basis of Therapeutics”, Ch. 1 p1).

It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the oncogenic disorder of interest will vary with the severity of the condition to be treated and with the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in “Remington's Pharmaceutical Sciences,” 1990, 18th ed., Mack Publishing Co., Easton, Pa. Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein.

In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of, for example, tablets, dragees, capsules, or solutions.

The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The proper dosage of a compound depends on various factors such as the type of disease being treated, the particular composition being used and the size and physiological condition of the patient. Therapeutically effective doses for the compounds described herein can be estimated initially from cell culture and animal models. For example, a dose can be formulated in animal models to achieve a circulating concentration range that initially takes into account the IC50 as determined in cell culture assays. The animal model data can be used to more accurately determine useful doses in humans.

Plasma half-life and biodistribution of the drug and metabolites in the plasma, tumors and major organs can also be determined to facilitate the selection of drugs most appropriate to inhibit a disorder. Such measurements can be carried out. For example, HPLC analysis can be performed on the plasma of animals treated with the drug and the location of radiolabeled compounds can be determined using detection methods such as X-ray, CAT scan and MRI. Compounds that show potent inhibitory activity in the screening assays, but have poor pharmacokinetic characteristics, can be optimized by altering the chemical structure and retesting. In this regard, compounds displaying good pharmacokinetic characteristics can be used as a model.

Toxicity studies can also be carried out by measuring the blood cell composition. For example, toxicity studies can be carried out in a suitable animal model as follows: 1) the compound is administered to mice (an untreated control mouse should also be used); 2) blood samples are periodically obtained via the tail vein from one mouse in each treatment group; and 3) the samples are analyzed for red and white blood cell counts, blood cell composition and the percent of lymphocytes versus polymorphonuclear cells. A comparison of results for each dosing regime with the controls indicates if toxicity is present.

At the termination of each toxicity study, further studies can be carried out by sacrificing the animals (preferably, in accordance with the American Veterinary Medical Association guidelines Report of the American Veterinary Medical Assoc. Panel on Euthanasia, Journal of American Veterinary Medical Assoc., 202:229-249, 1993). Representative animals from each treatment group can then be examined by gross necropsy for immediate evidence of metastasis, unusual illness or toxicity. Gross abnormalities in tissue are noted and tissues are examined histologically. Compounds causing a reduction in body weight or blood components are less preferred, as are compounds having an adverse effect on major organs. In general, the greater the adverse effect the less preferred the compound.

For the treatment of cancers the expected daily dose of a hydrophobic pharmaceutical agent is between 1 to 500 mg/day, preferably 1 to 250 mg/day, and most preferably 1 to 50 mg/day. Drugs can be delivered less frequently provided plasma levels of the active moiety are sufficient to maintain therapeutic effectiveness.

Plasma levels should reflect the potency of the drug. Generally, the more potent the compound the lower the plasma levels necessary to achieve efficacy.

VI. Cell-Based Therapeutics

Cell-based therapeutics rely on the ability of a cell, and in particular a stem cell, to differentiate along a specific cell lineage. The ability to direct lineage-specific differentiation can provide a virtually unlimited suply of source material for the treatment of diseases by tissue repair and regeneration. For example, hematopoetic stem cells have been used for many years to repopulate the bone marrow of animals, including humans, which have lost the ability to produce one or more blood cells. Methods for administering cell-based therapeutics are known to those of ordinary skill in the art. See, e.g., Stein, et al., International Publication No. WO 98/39427, published on Mar. 6, 1997, entitled “Gene Therapy Using Bone Marrow Transplants Transfected With Therapeutic Genes Under the Control of Tissue-Specific Promotors,” which is hereby incorporated by reference in its entirety, including all tables, figures, and claims.

### EXAMPLES

### Materials and Methods

### Example 1

### In vivo, In vitro, and Nuclear Transfer Embryos

Cryopreserved bovine in vivo embryos were purchased commercially. Bovine cumulus oocyte complexes were recovered from slaughterhouse ovaries by aspiration and in vitro matured in maturation medium at 39° C. in a 5% CO2 in air atmosphere as described in U.S. Pat. No. 5,453,366, issued to Sims et al. on Sep. 26, 1995, and/or U.S. Pat. No. 5,096,822, issued to Rosenkrans et al. on Mar. 17, 1992, each of which is hereby incorporated by reference in its entirety, including all tables, figures, and claims. A preferred maturation medium was prepared by combining 4.4 mL Medium 199 (Gibco-BRL 11150-042), 500 μL fetal calf serum (Hyclone), 50 μL Pen-Strep (Gibco-BRL 15140-122), 50 μL pyruvate (2 mg/mL in medium 199), 25 μL LH (Sioux Biochemical), and 5 μL estradiol (Sigma Chemical E-8875). Matured oocytes were inseminated by combining sperm and matured oocytes in a fertilization drop as described in U.S. Pat. Nos. 5,453,366 and/or 5,096,822. CR2 medium (CR1 medium supplemented with amino acids as described in U.S. Pat. No. 5,096,822)+6 mg/mL was preferred as a fertilization medium. Fertilized oocytes were matured in CR2 medium supplemented with 10% FCS and collected on five day post insemination.

### Example 2

### Cell Culture Conditions of Donor Cells, Embryonic Germ Cells and Embryonic Stem Cells

Bovine embryonic germ cells were derived from the genital ridge of 55 day old bovine fetuses and cultured in alpha-MEM (Gibco-BRL) supplemented with 10% fetal bovine serum (Hyclone) and 0.1 mM 2-mercaptoethanol (Gibco-BRL). Confluent culture dishes were passaged in 1× Trypsin-EDTA (Gibco-BRL) at least once before use in nuclear transfer. Bovine embryonic stem cells were derived from bovine nuclear transfer blastocyst that were on mitotically inactivated mouse fibroblast feeder cells in alpha-MEM (Gibco-BRL). Some ES cell cultures were supplemented with 50 ng/ml recombinant human leukemia inhibitory factor (rhLIF) (R & D Systems), 50 ng/ml fibroblast growth factor basic (bFGF) (R & D systems), and 1× Antibiotic-Antimycotic (Gibco-BRL).

### Example 3

### Nuclear Transfer Embryos

Matured oocyte complexes were pooled in HECM/HEPES and vortexed for three minutes to strip cumulus and placed in Hoescht medium 30 minutes prior to enucleation. Enucleation (removal of polar body and metaphase plate) was performed and oocytes were flashed with UV light (less than 10 seconds) to confirm enucleation. Enucleated oocytes were washed with HECM/HEPES and put back into a drop of CR2 medium prior to transfer of donor cells within the oocyte cytoplasma. Fusion of the enucleated oocyte and the donor cell was performed on a BTX 200 Electrocell fusion machine in a 500 μM fusion chamber by an electrical pulse of 90 V for about 15,sec. After fusion the resultant NTs were placed into CR2 medium plus fetal calf serum (Gibco-BRL) until activation. Fused NTs were activated between 4-9 hours later by exposing them to 5 μM ionomycin in HECM/HEPES supplemented with 1 mg/ml BSA for four minutes.

### Example 4

### RNA Isolation

Total RNA was isolated from single embryos (in vivo, in vitro, and nuclear transfer) using the RNeasy kit according to manufacturer's protocols (Qiagen). All buffers and reagents were supplied by the manufacturer with the exception of β-mercaptoethanol (Fisher Scientific). Briefly, in vitro and nuclear transfer embryos were collected (Day 5) and transfererd into 1.5 ml microcentrifuge tubes containing 350 μL RLT buffer and frozen at −80° C. prior to RNA isolation. In vivo bovine embryos were cryopreserved prior to RNA isolation and transferred into a 1.5 ml microcentrifuge containing 350 μL RLT buffer prior to RNA isolation. β-mercaptoethanol (0.145M) was added to the RLT buffer and embryos after incubation on ice. The embryos were homogenized by vortexing for 30 seconds. After addition of 70% ethanol (350 μl) the homogenized lysates were applied to the RNeasy mini spin column and centrifuged for 15 seconds at 10,000 rpm (discarded flow-through). The wash buffer RW1 (7001) was applied to the RNeasy column and centrifuge for 15 seconds at 10,000 rpm (discarded flow-through). The RNA was precipitated by addition of 500 μl of RPE buffer onto the RNeasy column and centrifuged for 15 seconds at 10,000 rpm (discarded flow-through). An additional 500 μl of RPE buffer was applied onto the RNeasy column and centrifuged for two minutes at maximum speed to dry the RNeasy membrane. The RNeasy column was transferred into a new 1.5 ml collection tube (supplied by manufacturer) and 30 μl of Rnase-free water was applied directly onto the RNeasy membrane. The RNeasy membrane was centrifuge for one minute at 10,000 rpm to elute the RNA.

Alternatively, RNA is isolated from single embryos using the Micro RNA Isolation Kit (Stratagene) according to the manufacturer's protocols. Briefly, individual embryos were incubated in 200 μL of denaturing buffer and 1.6 μL of β-mercaptoethanol at room temperature for 5 minutes. Extraction was performed in 20 μL of 2M sodium acetate, 200 μL phenol, and 60 μL chloroform:isoamyl alcohol. The aqueous layer was collected and mixed with 1 μL glycogen (10 mg/mL), and precipitated with 200 μL isopropanol. The sample was washed with 70% ethanol, air dried, and resuspended in 16 μL RNase-free water, 2 μL DNase I reaction buffer, 1 μL RNasin, and 1 μL DNase I. The resulting solution was incubated at 37° C. for 30 minutes, the nucleic acid was precipitated, and the resulting pellet resuspended in 10 μL DEPC-treated water.

### Example 5

### First-Strand Synthesis of cDNA and Amplication of cDNA

Total RNA isolated from single in vivo, in vitro, and nuclear transfer embryos was used to produce cDNA with the SMART PCR cDNA synthesis kit following manufacturer's protocol (Clontech). Briefly, 3 μl of RNA sample was combined with 1 μl of cDNA synthesis (CDS) primer (10 μM) (5′-AAGCAGTGGTAACAACGCAGAGTACT₍₃₀₎ N₋₁ N-3′; N=A, C, G, OR T; N1=A, G, or C) and 1 μl of SMART II Oligonucleotide (10 μM) (5′-AAGCAGTGGTAACAACGCAGAGTACGCGGG-3′) into a 0.5 ml microcentrifuge tube. Contents were mixed and briefly centrifuged prior to incubation at 70° C. in a therma cycler for 2 minutes. After incubation, the tubes were spun briefly in a microcentrifuge to collect contents at the bottom. The tubes were kept at room temperature. The following was added to each reaction tube: 2 μl of 5× First-Strand Buffer (250 mM Tris-HCl (pH 8.3), 375 mM KCl, 30 mM MgC12), 1 μl of DTT (20 mM), 1 μl of 50×dNTP (10 mM each dNTP), and 1 μl of MMLV reverse transcriptase (Superscript II, 200 units/μl; Gibco-BRL). Microcentrifuge tubes were gently mixed and then spun in a microcentrifuge. The reaction mixtures were overlayed with one drop of mineral oil (to prevent evaporation) and incubated at 42° C. for 1 hour in a therma cycler. The first-strand reaction product was diluted by adding 40 μl of TE buffer (10 mM Tris (pH 7.6), 1 mM EDTA). Microcentrifuge tubes were heated at 72° C. for 7 minutes to inactivate the reverse transcriptase. For amplication of cDNA, the PCR thermal cycler was preheated to 95° C. For each embryo cDNA sample, 10 μl of single-stranded cDNA was transferred into a 0.5 ml microcentrifuge tube. The following was added to each reaction tube (supplied by manufacturer; Clontech): 74 μl of sterile deionized H₂O, 10 μl of 10× Advantage 2 PCR Buffer, 2 μl of 50×dNTP (10 mM each dNTP), 2 μl of PCR primer (10 μM) (6FAM-5′-AAGCAGTGGTAACAACGCAGAGT-3′; modified at the 5′ end with 6FAM), and 2 μl of 50× Advantage 2 Polymerase Mix. Contents in microcentrifuge tubes were mixed well and spun briefly in microcentrifuge. The reaction mixtures were overlayed with two drops of mineral oil (to prevent evaporation). Thermal cycling paramaters were as follows: one cycle at 95° C. for 1 min, followed by 25 cycles at 95° C. for 15 sec, 65° C. for 30 sec, and 68° C. for 6 min. To confirm amplification of cDNA was successful, a 5 μl aliquot of each sample was electrophoresed on a 1.0% agarose/ethidium bromide gel in 1× TBE buffer. Typical results, indicative of a successful PCR according to the manufacturer (Clontech) had a moderately strong smear of cDNA from 0.5 to 6 kb and several bright bands corresponding to abundant transcripts.

### Example 6

### Linear Amplification of RNA using T7 polymerase by Reverse Transcription (RT)

10 μL of purified RNA was mixed with 1 μL T7-oligo(dT) primer (5′-TCTAGTCGACGGCCAGTGAATTGTAATAGCACTCACTATAGGGCGT₂₁-3′) (0.5 mg/mL) to initiate first strand synthesis. The primer and RNA were incubated at 70° C. for 10 minutes, followed by incubation at 42° C. for 5 minutes. Next, 4 μL of first strand reaction buffer (2 μL 0.1M DTT, 1 μL 10 mM dNTPs, 1 μL RNasin (Promega), and 1 μL SuperScript II (Life Technologies) were added, and the resulting mixture incubated at 42° C. for 1 hour. Subsequently, 30 μL of second strand buffer (3 μL 10 mM dNTPs, 4 μL DNA polymerase I, 1 μL E. coli RNase H, 1 μL E. coli DNA ligase, and 92 μL RNase-free water) was added, and the mixture incubated at 16° C. for 2 hours, followed by addition of 2 μL T4 DNA polymerase and incubation at 16° C. for 10 minutes. cDNA was extracted with pehnol-chloroform, and washed 3 times with 500 μL on a Microcon-100 column (Millipore).

Amplification was accomplished using the Ampliscribe T7 Transcription Kit (Epicentre Technologies) according to manufacturer's instructions. Briefly, 8 μL of cDNA was added to 2 μL of 10×Ampliscribe T7 buffer, 1.5 μL each of 100 mM ATP, CTP, GTP, and UTP, 2 μL 0.1 M DTT, and 2 μL T7 RNA polymerase, and incubated at 42° C. for 3 hours. The resulting RNA was washed 3 times using a Microcon-100 column, collected, and dried to 10 μL.

RNA from the first amplification round was mixed with 1 μL random hexamers (Pharmacia) (1 mg/mL), incubated at 70° C. for 10 minutes, chilled on ice, then brought to room temperature. To this sample, 4 μL of first strand buffer, 2 μL 0.1 M DTT, 1 μL 10 mM dNTPs, 1 μL RNasin, and 1 μL SuperScript II were added, and the resulting mixture incubated at room temperature for 5 minutes, followed by 37° C. for 1 hour. Subsequently, 1 μL RNase H was added, followed by an incubation at 37° C. for 20 minutes, 95° C. for 2 minutes, then chilling on ice. For second strand cDNA synthesis, 1 μL T7-oligo(dT) primer (0.5 mg/mL) was added, and the mixture incubated at 70° C. for minutes and 42° C. for 10 minutes. To this solution, 30 μL of second strand synthesis buffer, 3 μL 10 mM dNTPs, 4 μL polymerase I, 1 μL E. Coli RNase H, and 90 μL RNase-free water was added, followed by incubation at 16° C. for 2 hours. 2 μL of T4 DNA polymerase was then added, followed by incubation for 10 minutes. Double stranded DNA was then extracted with 150 μL phenol-chloroform, and washed using a Microcon-100 column. The resulting cDNA could be used for a additional rounds of in vitro transcription and RNA amplification.

### Example 7

### Differential Display

The identification of differentially expressed RNAs from in vivo, in vitro, and nuclear transfer derived embryos was performed using reagents supplies in the Delta Differential Display kit and following manufacturer's protocol (Clontech). Briefly, 2 μl of PCR amplified in vivo, in vitro, or nuclear transfer single embryo cDNA were used as templates and amplified in the presence of [α-³³P]dATP (50 nM) (Amersham), 1.0 μM each of arbitrary “P” and oligo(dT) primers, 2× KlenTaq PCR reaction buffer, 50 μM each dNTP, and 2.5× Advantage KlenTaq Polymerase Mix. Thermal cycling conditions were as follows: One cycle at 94° C. for 5 min, 40° C. for 5 min, and 68° C. for 5 min; two cycles at 94° C. for 2 min, 40° C. for 5 min, and 68° C. for 5 min; followed by 25 cycles at 94° C. 1 min, 60° C. 1 min, and 68° C. for 2 min; final extension was performed for an additional 7 min at 68° C. Differential display products were loaded onto 6% denaturing polyacrylamide gels and electrophoresed at 70W for a minimum of seven hours. Polyacrylamide gels were transferred to Whatman paper and dried under vacuum at 75° C. for 60 min. X-ray film was exposed to the gels at room temperature overnight with intensifying screens. Differentially expressed bands were excised from dried gels and transferred into 0.6-ml microcentrifuge tubes. The gel slices (along with the Whatman paper) were soaked in 100 μl H₂O for 10 minutes, followed by heating at 99.9° C. in thermal cycler for 15 min. Microcentrifuge tubes were spun for 2 min to collect condensation and pellet the gel and paper debris. The supernatants were transferred to new 0.6-ml microcentrifuge tubes and 10 μl of 3M NaOAC, 5 μl of glycogen (10 mg/ml) and 450 μl of 100% ethanol were added. Samples were placed at −80° C. for 30 min and spun at maximum speed in microcentrifuge for 10 min to pellet DNA. Supernatants were removed and the DNA pellets were washed with 200 μl of ice-cold 85% ethanol. Samples were spun briefly and residual ethanol was removed. DNA pellets were resuspended in 20 μl of sterile H₂O. Eluted bands were stored at −20° C. Differentially expressed bands were reamplified using primer(s) used in the original differential display PCR. Each 12 μl reaction contained 2 μl of eluted DD band, 0.5 μM each primer, 0.8 μM each dNTP, 1.5 mM MgCl₂, 1×PCR buffer (AmpliTaq) and 0.2 units AmpliTaq DNA Polymerase (Perkin Elmer). Thermal cycling conditions were: 3 min at 94° C., followed by 20 cycles of 1 min at 94° C., 1 min at 60° C., 1 min at 72° C., and a final extension of 4 min at 72° C. Re-amplication products were cloned into pGEM-T vector (Promega) and sequenced using ABI Prism BigDye terminator cycle sequencing kit (PE Applied Biosystems) and automated nucleotide sequencer (GeneSys). The resulting sequencing data were aligned and analyzed using SeqMan (DNASTAR), and BLAST (Basic Local Alignment Search Tool).

### Example 8

### Embryonic Germ (EG) Cell cDNA Library

A bovine EG cell cDNA library was custom made by Stratagene (La Jolla, Calif.). Briefly, bovine EG cells isolated from the genital ridges of a slaughterhouse bovine fetus were grown at Infigen, Inc., in α-MEM (Gibco BRL) supplemented with 10% fetal bovine serum (Gibco-BRL) and 0.1 mM β-mercaptoethanol (Gibso BRL). cDNA was synthesized from RNA isolated from 80×10⁶ EG cells. For directional cloning an Xho I site was introducted at the 3′ end of the cDNA, by using an oligo(dT) primer contiaing an Xho I site for priming first strand synthesis, and ligating an EcoRI adapter to the 5′ end of the double-stranded cDNA. The directional cDNA was then ligated into lambda arms of the Uni-ZAP vector (Stratagene) cut with EcoRI and XhoI. The average insert size is 1.0 kb with a size range of 0.5-2.2 kb. The estimated amplified titer is 1.2×1010 pfy (plaque forming units)/ml, representing 10⁶ recombinants. In vivo mass excision of the pBluescript phagemid from the Uni-ZAP XR vector was performed to generate a subtraction library. Briefly, overnight cultures of XL-1Blue MRF′ and SOLR cells grown in LB broth supplemented with 0.2% (w/v) maltose and 10 mM MgSO₄ were spun down and resuspended in 10 mM MgSO₄ to an OD₆₀₀ of 1.0 (8×10⁸ cells/ml). In a 50 ml centrifuge tube 10⁷ pfu of the amplified lambda bacteriophage library was combined with 10⁸ XL1-blue MRF′ cells and 10⁹ pfu of ExAssist helper phage and incubated at 37° C. for 15 minutes. LB broth was added to the mixture and incubated at 37° C. for 3 hours with shaking. The centrifuge tube was heated at 65° C. for 20 minutes, followed by spining at 1000×g for 10 min. The supernatant was decanted into a new sterile centrifuge tube, diluted, and combined with 200 μl of SOLR cells (previously diluted to 8×10⁸ cells/ml) in a 1.5 ml microcentrifuge tube and incubated at 37° C. for 15 min. A portion of the cell mixture was plated onto LB-ampicillin agar plates (100 μg/ml) and incubated overnight at 37° C. Individual colonies were picked from the agar plates and transferred in single wells of a 96 well block containing 1.3 ml LB broth supplemented with 100 μg/ml ampicillian and incubated for 24 hours in a shaking 37° C. incubator. The bacterial cells were harvested by centrifugation for 5 min at 1500×g. Medium was removed by inverting the block. Plasmid DNA was isolated using the R.E.A.L. Prep 96 Plasmid kit (Qiagen) following manufacturer's protocol and supplied reagents. Briefly, bacterial pellets were resuspended in Buffer R2 and lysed after the addition of Buffer R3. The 96 well blocks were placed in a boiling water bath for 5 min and cooled down to room temperature by incubating on ice for 10 min. The bacterial lysates were transferred to the wells of the QIA filter 96 well plate and transferred to another 96 well block by vacuum. The DNA was desalted and concentrated by adding 0.7 volumes of room temperature isopropanol to each well and inverted to mix. The plasmid DNA was pelleted by centrifugation at 2500×g for 15 min. DNA pellets were washed with 0.5 ml cold 70% ethanol and centrifuged to reconcentrate the pellets. Plasmid DNA pellets were air dried and redissolved in 50 μl of Tris-EDTA, pH 8.0.

### Example 9

### Sequencing of Bovine EG cDNA/EST Library

Sequencing of cloned cDNA inserts from the EG cDNA library was performed using the ABI Prism Big Dye Terminator cycle Sequencing kit (PE-Biosystems) following manufacturer's protocol and supplied reagents. Sequencing reactions were electrophoresed and analyzed using an automated nucleotide sequencer (Genesys9600 and/or Perkin Elmer ABI 377). The resulting sequencing data were aligned and analyzed using SeqMan (DNASTAR), and BLAST (Basic Local Alignment Search Tools).

### Example 10

### Macroarray Preparation and Use

Insert cDNA samples from the bovine EG cDNA library were amplified by PCR using flanking vector specific primers T7 and T3. Each 50 μl reaction contained 2 μl of DNA template, 1× AmpliTaq Reacton buffer, 1.5 mM MgCl₂, 0.5 μM each primer, 0.8 μM each dNTP, and 0.2 units AmpliTaq DNA Polymerase (Perkin Elmer). Thermal cycling conditions were: 3 min at 94° C., followed by 30 cycles of 1 min at 94° C., 1 min at 60° C., 1 min at 72° C., and a final extension of 4 min at 72° C. Following PCR amplification of the clone inserts, the PCR products were spotted onto neutral nylon membranes soaked in 0.5 M NaOH/1.5 M NaCl using the HDR tool and Biomek 2000 (Beckman). After spotting onto nylon membranes, the DNA was neutraliszed in 1.0 M Tris-Cl pH 7.4/1.5 M NaCl. DNA was cross-linked by WV irradiation. Nylon membranes were pre-hybridized at 65° C. for four hours in modified Church buffer containing 0.25 M Na₂HPO₄ (pH 7.2), 7% SDS, 1 mM EDTA and 0.5 mg/ml denatured salmon sperm DNA. The membranes were hybridized in Church Buffer at 65° C. for a minimum of 16 hours using ethanol precipitated amplified probe (previously described). Nylon membranes were washed twice in 2×SSC/0.1% SDS at room temperature with gentle agitation. To detect the hybridization of amplified probe to target, the ECF Signal Amplificaiton module (Amersham Pharmacia Biotech) was used. Briefly, after a blocking step, the membrane was incubated with an anti-fluorescein alkaline phosphatase (AP) conjugate (amplified probe contained 6FAM). After washing off the excess conjugate, detection reagent was added and probe-bound AP catalyzed the conversion of the detection reagent to a highly fluorescent product. The fluorescent product was visualized using a signal FluorImager (Molecular Dynamics).

### Example 11

### Microarray Use

cDNA probes were labeled with Cye-3 and Cye-5 dyes using the Superscript Choice System for cDNA synthesis (Gibco-BRL) according to manufacturer's instructions. DNA affixed on a glass slide microarray was hybridized, and scanned using a Genepix 4000 Scanner and integrated software (Axon Instruments, Inc.). Formamide-based hybridization conditions at 42° C. were preferred over aqueous solutions containing either polyethylene glycol or dextran sulfate. Denhardt's Solution was preferred as a blocking reagent, although SDS, salmon sperm DNA, tRNA, or Cot, DNA may be used. Information related to intensity values, intensity ratios, normalization constants, and confidence intervals was assigned to each target. Data was typically viewed as a normalized ratio (Cye-3/Cye-5), in which significant deviations from 1 (no change) are indicative of increased (>1) or decreased (<1) levels of gene expression.

### Example 12

### Developmentally Competent and Developmentally Incompetent Cell Lines

The competence of 59 bovine cell lines previously used in nuclear transfer procedures were compared for the ability to produce live-born animals. The donor cell lines exhibit a range of competencies for successful reprogramming. Critical variables correlated with these competency ranges include culture media, number of passages and days in culture. The minimum standard for competency was defined as producing pregnancy initiation rates of greater than 50%, 90 days gestation, and live birth. 90 days appears to be a key indicator of live birth for cattle, as over 50% of NT fetuses that reach this mark survive full term. In tables 1A and B, embryos generated from a competent donor cell line will be identified by a ‘+’ while ‘−’ embryos generated from an incompetent donor cell line will be distinguished by a ‘−’. For example, BFEG⁺ refers to a competent EG cell line, while BFEG⁻ refers to an incompetent EG cell line.

### Example 13

### Assessing the Effect of Changes in a Nuclear Transfer Protocol on Developmental Competence

Two cell lines in particular illustrate the differences in range of competencies: BFES⁺ and BFES⁻. These are embryonic stem cell lines cultured under different conditions and used to produce nuclear transfer embryos. Line BFES⁻ represents a stem cell line cultured using conditions that produced donor ES cells used for greater than 50,000 nuclear transfers. From this pool of NT embryos, 2000 were transferred into recipients over a two year period, and all failed to develop beyond 55 days in utero. By contrast, using novel culture procedures aimed at minimizing differentiation and maximizing embryonic stem (ES) cell growth, Infigen generated stem cell line BFES⁺ to use as nuclear donors. Briefly, bovine embryonic stem cells were derived from bovine nuclear transfer blastocyst that were on mitotically inactivated mouse fibroblast feeder cells in alpha-MEM (Gibco-BRL). Some ES cell cultures were supplemented with 50 ng/ml recombinant human leukemia inhibitory factor (rhLIF) (R & D Systems), 50 ng/ml fibroblast growth factor basic (bFGF) (R & D systems), and 1× Antibiotic-Antimycotic (Gibco-BRL).

These cells were used to construct embryos that have sustained pregnancies greater than 90 days in 10% of the transferred embryos. This data suggests that developmentally competent reprogramming can be enhanced by culture conditions of donor cell lines.

### Example 14

### Identifying Molecular Events Related to Developmental Competence by Immunoblot Analysis

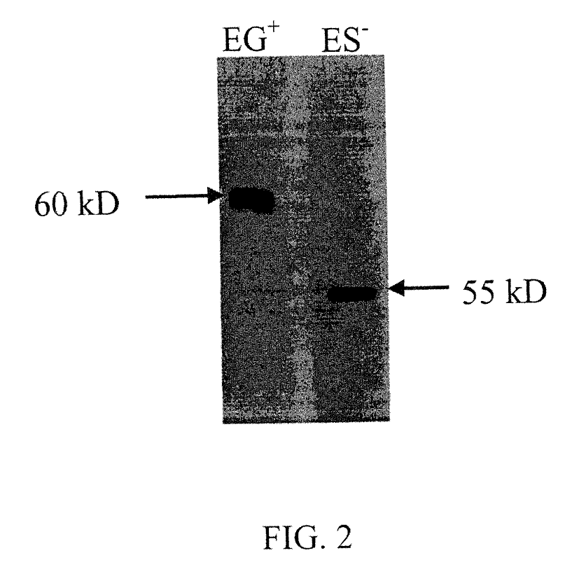

Immunoblot analysis was performed using standard protocols and essentially as described in Harlow and Lane (Antibodies: A Laboratroy Manual, pgs 471-506). Briefly, cells were grown as described previously and resuspended in approxiamtely 10 volumes of sample buffer (2% SDS, 100 mM DTt, 60 mM Tris, pH 6.8, 0.1% bromophenol blue). Samples were boiled for 5 minutes and immediately loaded onto 10-20% Tris/glycine SDS-polyacrylamide gradient gels. Proteins were separated by electrophoresis at 100-125 V until the dye front reached the bottom of the gel. Proteins were transferred to nitrocellulose in transfer buffer (50 mM Tris, 380 mM glycine, 0.1% SDS, 20% methanol) at 100 volts for 1 hour. Mouse anti-histone deacetylase 2 primary antibody (Santa Cruz Biotechnology, Inc.) was used at a dilution of 1:100 in blocking solution (5% wt/vol nonfat dry milk, 0.2% Tween 20, 0.02% sodium azide in PBS). Goat anti-mouse secondary antibody conjugated to horse radish peroxidase (Santa Cruz Biotechnology, Inc.) was used at a dilution of 1:500, also in blocking solution. Detection was accomplished using ECL+Western botting detection system (Amershampharmacia, cat. # RPN2132). Immunoblot analysis has identified a potentially novel, 55 kD isoform of bovine histone deacetylase 2 (HDAC2) present in bovine BFES⁻ donor cells, but absent in competent bovine BFEG⁺ donor cells. By contrast, an approximately 60 kD band is detected in bovine BFEG⁺ cells but not BFES⁻ cells. (FIG. 2).

In addition, we have determined that histone deacetylase 1 is present in bovine BFEG⁺ cell lines but absent in BFES⁻ cells (data not shown). It has been presumed that successful reprogramming requires extensive chromatin remodeling, a process highly dependent on histone acetylases and deacetylases. See, e.g., Liang and Pardee, 1992, Science 257: 967-971; Wilmut, 1998, Scientific American 279: 58-63. Taken together, these observations suggest that donor cells can impact reprogramming and developmental competence by activating or deactivating genes and/or biochemical pathways that in turn could enhance or disrupt the reprogramming process. For example, novel deacytalses may alter chromatin remodeling kinetics.

### Example 15

### Identifying Molecular Events Related to Developmental Competence by Differential Display

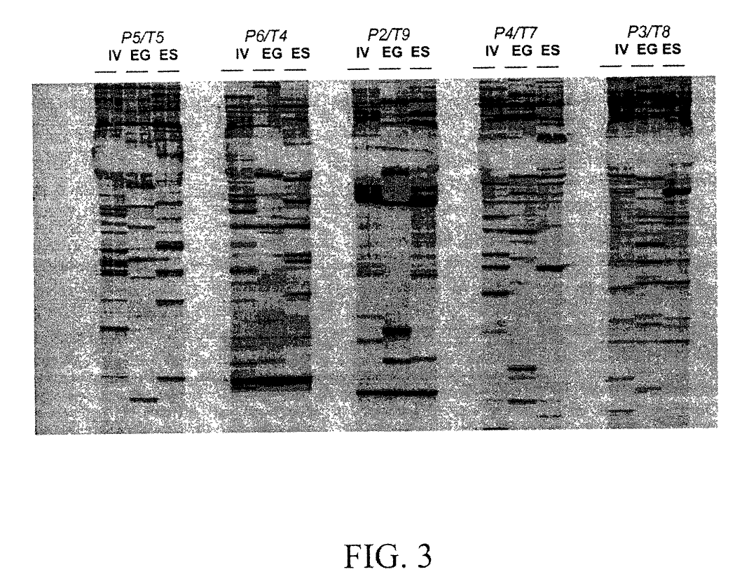

Differential display (DD) was used to compare mRNA profiles of single embryos generated by nuclear transfer to in vivo embryos. The nuclear transfer embryos were reconstructed from a competent cell line [BFES⁺] and an incompetent cell line [BFEG⁻]. DD was used to calculate the percentage of bands conserved between single day 7 in vivo embryos, and single day 5 NT embryos generated from incompetent EG⁻, and competent ES⁺ donor cells. It is important to note that day 7 bovine in vivo embryos and day 5 bovine NT embryos have identical morphology, the same number of cells, and are considered to be at the optimal stages for comparison. This analysis revealed a 73% difference in banding patterns between day 7 in vivo embryos and day 5 BFES⁺ embryos, and a 74% difference between day 7 in vivo embryos and day 5 BFEG⁻ NT embryos (FIG. 3). A band was considered different if present in the in vivo sample but absent in either of the NT samples.

These results suggest that for individual embryos, mRNA expression patterns of embryos reconstructed with donor nuclei may not be converted to blastocyst patterns that represent the best model of successful reprogramming (i.e., in vivo produced embryo). These data further suggest a potentially large number of genes may have altered expression levels in NT reconstructed embryos when compared to in vivo embryos. The observation that nuclear transfer embryos reconstructed from competent cell lines may also have distinct DD patterns from in vivo embryo patterns may partially account for the poor efficiencies of the nuclear transfer process.

By contrast, researchers using differential display protocols demonstrated that expression patterns are highly conserved (95%) between in vivo, IVF, and NT embryos, suggesting that developmental programs very similar to those detected for in vivo embryos can be established after nuclear transfer. See DeSousa et al., 1999, Cloning 1: 63-69. However, this analysis was based on single embryo equivalent representations obtained from pools of embryos. This method can mask differences between individual embryos, which in turn may account for individual embryo differences during development and the low live birth rates observed by artisans. For example, if 20% of NT embryos were developmentally competent, pooling template from 5 embryos likely would produce results substantially similar to results from a competent in vivo embryo. The data provided herein suggest that the vast majority of individual, NT reconstructed embryos may not reproduce expression patterns similar to in vivo patterns. The ability to monitor single embryos is critical to minimize genetic noise that might obscure underlying reproducible expression patterns. Since differences appear to be readily detectable at a single embryo level, deficiencies and/or differences in the mRNA profiles of NT embryos when compared to in vivo embryos ultimately will help identify genes/mechanisms responsible for low (live birth) efficiencies and developmental problems.

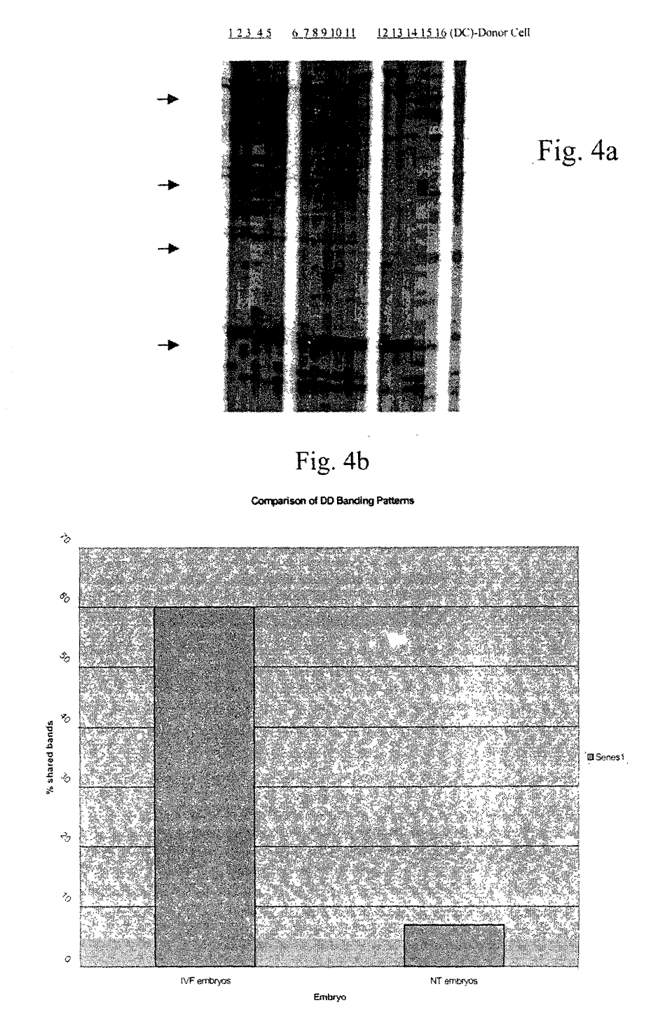

FIG. 4 describes comparing banding patterns generated by differential display (FIGS. 4A & B) between five individual day 7 in vivo embryos (lanes 1-5); six individual day 5 IVF embryos (lanes 6 and 11)]; five individual embryos reconstructed by NT [three day 5 embryos (lanes 12-14), one day 7 (lane 15) and one day 8 (Lane 16)] using a developmentally incompetent cell line, and the developmentally incompetent donor cell (DC) line. Day 7 bovine in vivo embryos and day 5 bovine NT embryos have identical morphology, the same number of cells, and are considered to be at the proper stages for accurate comparison. Briefly, RNA from each embryo was isolated, reverse transcribed and amplified using the protocol described previously. Differential display reactions were performed using one of 15 primer pairs. Analysis of banding patterns (FIG. 4A) revealed 122 bands present in all 5 individual day 7 in vivo embryo samples. Seventy three (60%) of the 122 bands were also identified in five day 5 individual IVF produced embryos. (Less than 10% of the bands were present in the sixth, day 6 IVF embryo, lane 11.) In sharp contrast only 9 (7%) of the 122 bands were identified in all of the day 5 individual embryos reconstructed by NT (FIG. 4B). (The number of matching bands was less for day 7 and day 8. The bands that did match showed altered expression patterns.) Moreover, these were the only 9 bands (of over 700 identified bands observed cumulatively in the 5 NT samples) present in all day 3 NT reconstructed embryos. This indicates tremendous heterogeneity among individual NT produced embryos collected at precise times after activation and not observed in either in vivo or IVF embryos. These banding patterns further support the hypothesis that low NT efficiencies may be due partly to improper reprogramming, exemplified by the different banding patterns between individual embryos reconstructed by NT. Most importantly, these data indicate that genes expressed at high levels in developmentally competent embryos, but at low or undetectable levels in developmentally incompetent embryos can be identified and used to determine an idealized expression pattern. The consistent amplification of identical bands in all individual in vivo embryo samples and all individual IVF embryo samples further demonstrates that the embryo harvesting and amplification protocols described herein are reliable for detecting gene products whose expression levels are either relatively high or low.

These results also suggest that, for individual embryos, mRNA expression patterns of embryos reconstructed with donor nuclei may not be converted to blastocyst patterns that represent a best model of successfuil reprogramming (i.e., in vivo produced embryo). This is further supported in a report by Eckert and Niemann (Mol. Human Reprod. 4: 957-65, 1998) who identified perturbations in mRNA expression patterns specific to the LIF-LIF receptor system in embryos generated in vitro and possibly correlated with improper blastocyst development. By contrast, de Sousa et. al. used differential display protocols to demonstrate that expression patterns are highly conserved (˜95%) between in vivo, IVF, and NT embryos, suggesting that developmental programs very similar to those detected for in vivo embryos can be established after nuclear transfer. However, their analysis was based on pools of embryos. This method can mask differences between individual embryos, which in turn may account for individual embryo differences during development and the low live birth rates observed. For example, if 20% of NT embryos were developmentally competent, pooling template from 5 embryos likely would produce results substantially similar to results from a competent in vivo embryo. The data provided herein suggests the vast majority of individual, NT reconstructed embryos may not reproduce expression patterns similar to in vivo patterns. The ability to monitor single embryos is critical to minimize genetic noise that might obscure underlying reproducible expression patterns. Since differences appear to be readily detectable at a single embryo level, deficiencies and/or differences in the mRNA profiles of NT embryos when compared to in vivo embryos may help identify genes/mechanisms responsible for low (live birth) efficiencies and developmental problems.

### Example 16

### Identifying Molecular Events Related to Developmental Competence by Differential Display Using Microarrays

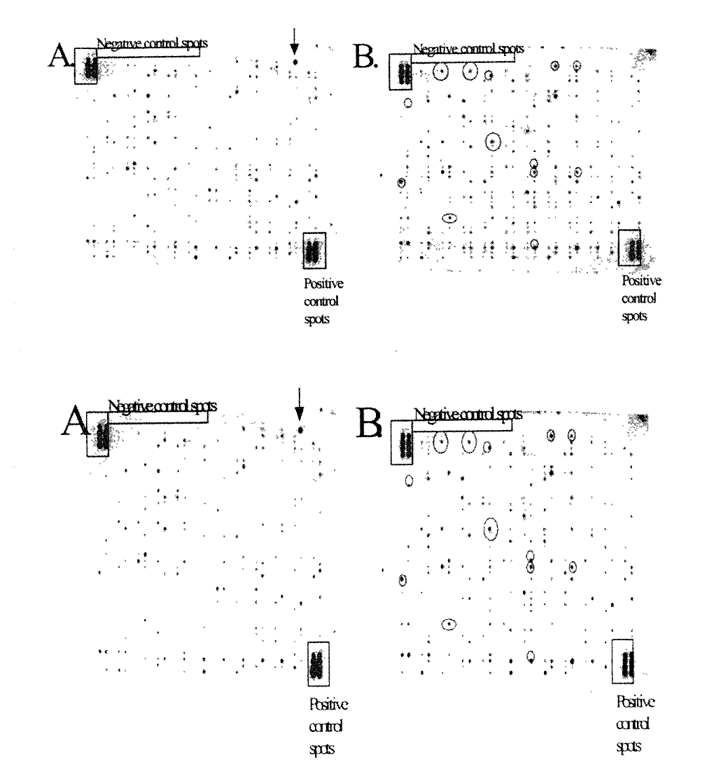

Though differential display can be used to identify reprogramming differences between embryos generated by NT and those produced in vivo, the method cannot be used in a high throughput format and cannot be performed on a sufficiently broad scale to characterize reprogramming at a molecular level. Limitations include very labor intensive procedures after identification of differentially expressed bands and confirmation of differential expression. Also, standard differential display does not allow genomic scale comparison and sophisticated statistical analysis of expression data, and thus prevents ‘rapid’ elucidation of comprehensive molecular patterns and relationships. To reconfirm our results and compensate for the limitations of differential display cDNA microarray technology was used to investigate and compare expression profiles of single in vivo and in vitro derived embryos (FIG. 5).

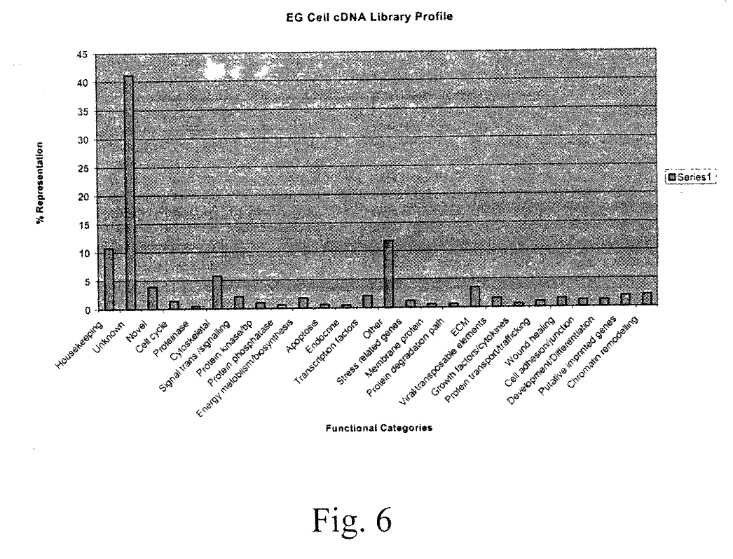

The microarray was comprised of cDNA clones representing numerous functional classes and gene families, including unknown ESTs, genes putatively associated with reprogramming (SNF2), cell cycle progression (quiescen, cyclins), cell adhesion-extracellular matrix (collagen, fibronectin), apoptosis (p53), imprinting (Igf2 and Igf2r), transcription (STAT), embryonic signaling (interferon tau), and signal transduction (JAK) (FIG. 6).

To amplify and detect hybridization signals, protocols for linear and exponential amplification of cDNA representing mRNA from a single embryo were employed. See, e.g., Van Gelder et al., U.S. Pat. No. 5,716,785, issued on Feb. 10, 1998, which is hereby incorporated by reference, including all tables, figures, and claims. After incorporating appropriate controls 744 PCR amplified arrayed cDNA clones obtained from an arrayed EG cell cDNA library were spotted onto nylon membranes, which in turn were probed with cDNA representations of a single in vivo embryo and 4 single embryos generated by NT from 1 competent (+), 1 unknown, and 2 incompetent (−) cell lines. A similar comparison of conservation after hybridization has confirmed basic plus/minus differences in expression patterns between individual embryos. Embryos generated from the 2 incompetent and unknown cell lines ranged in similarity from 18-85%, while the embryo generated from the competent cell line had a similarity to the in vivo embryo of 88%. Even a 3% difference in similarity between NT embryos generated from competent and incompetent (+ and −) donor cell lines represents 22 of the 744 genes screened with detectable (on/off) plus/minus differences. Studies in Phase II propose measuring expression levels of 10,000 genes. These data suggest that, from this pool, as many as 300 genes may have detectable plus/minus expression levels, and many more will likely have less subtle, but measurable differences. These results are the first visualization of broad changes in mRNA expression patterns between individual nuclear transfer and in vivo embryos.

Nucleotide sequences analyzed by the methods described herein are provided in Tables 2 and 3. Each sequence was determined to have a positive, negative, or neutral association with successful cellular reprogramming. The individual nucleotides in these sequences are provided as A=adenine; T=thymine; G=guanine; C=cytosine; N=nucleotide not determined. Individual sequences in Table 2 begin with a sequence identifier, and are separated by blank lines, while those in Table 3 are separated by two blank lines.

### Example 17

### Statistical Analysis of Molecular Events Related to Developmental Competence

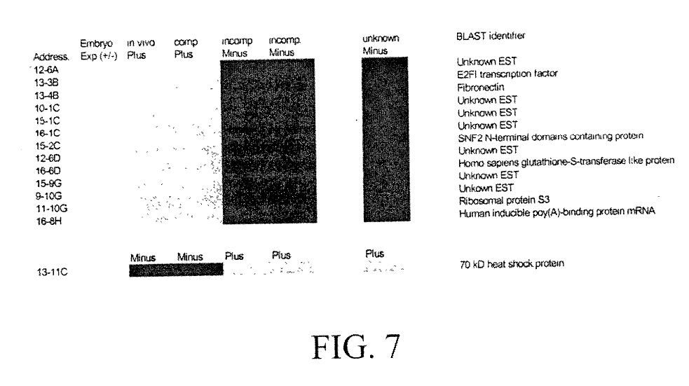

Clustering analysis was used to identify an idealized expression pattern for a developmentally competent embryo. 14 genes uniquely associated with reprogramming were identified (FIG. 7). The following EST sequences were identified as being associated with reprogramming: 990809a-88, 990726a-13, 990726a-14, 990726a-14, 990729a-1, 990729a-13, 990928a-9, 990928a-10, 990928a-65, 991108a-13, 991108a-14, 991108a-87, 991115a2-13, 991115a2-24, and 991115a2-92. Thirteen genes were always expressed in both the in vivo and competent cell derived embryos, but not in any of the embryos generated from incompetent cell lines. One gene was not expressed in the 2 competent embryo samples, but was detected in all three incompetent samples. The embryo derived from the unknown cell line had an expression pattern that matched 100% the embryos generated from the 2 incompetent cell lines. Transfer of embryos generated from the unknown cell line into recipient heifers failed to meet the criteria of a developmentally competent cell line (no pregnancy initiation was detected for any of the recipients), suggesting that it may be feasible to 1) identify an ‘idealized expression pattern’ of genes representing developmentally competent reprogramming and 2) identify genes that can be used to predict developmental viability. Gene expression differences between in vivo and nuclear transfer embryos are likely to contribute to the high inefficiencies associated with nuclear transfer cloning and potentially represent reprogramming deficiencies.

### Example 18

### Identifying Developmentally Competent and Incompetent Nuclear Donor Cell Lines, and Developmentally Competent and Incompetent Nuclear Transfer Embryos

In order to determine if a nuclear donor cell line is comprised of developmentally competent or incompetent cells, one or more cells are separated from the cell line and used as nuclear donors to provide one or more nuclear transfer embryos by the methods described herein. RNA or protein is isolated, and optionally amplified, for identification of molecular markers that indicate developmental competence or incompetence. If the embryos are cultured in vivo or in vitro to at least the two cell stage, the embryo can be divided into two or more portions, such that at least part of the embryo is retained for possible implantation into a maternal host.

The invention illustratively described herein may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

The inventions illustratively described herein may suitably be practiced in the absence of any element or elements, limitation or limitations, not specifically disclosed herein. Thus, for example, the terms “comprising”, “including,” containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

Table 1A and B: illustrates data concerning the developmental competence of 59 different nuclear donor cell lines.

TABLE 1A
Incompetent cell lines.

| | Pregnancy | Full | Number of ongoing |
|---|---|---|---|
| Cell Line: | Initiation (%) | term calves | pregnancies |
| BF101/65-c238-15 | 0 | 0 | 0 |
| BF104/15-16 | 0 | 0 | 0 |
| BF108-17 | 10 | 0 | 1 |
| BF12n2-18 | 15.79 | 0 | 0 |
| BF13n1-19 | 10.00 | 0 | 0 |
| BF15-20 | 13.64 | 0 | 0 |
| BF15-21 | 18.75 | 0 | 0 |
| BF15-22 | 25.00 | 4 | 0 |
| BF15-23 | 4.55 | 0 | 0 |
| BF15-24 | 19.77 | 0 | 0 |
| BF15-25 | 20.25 | 0 | 0 |
| BF15-26 | 40.00 | 0 | 0 |
| BF15-27 | 20.00 | 1 | 0 |
| BF15-28 | 36.36 | 0 | 0 |
| BF15-29 | 1.92 | 0 | 0 |
| BF15-30 | 13.24 | 0 | 0 |
| BF15-31 | 29.41 | 0 | 0 |
| BF15n4-32 | 22.22 | 0 | 0 |
| BF15n7-33 | 37.50 | 0 | 2 |
| BF19-34 | 33.33 | 1 | 0 |
| BF24/15-35 | 25.00 | 2 | 0 |
| BF33/21-36 | 0.00 | 0 | 0 |
| BF65-37 | 27.27 | 0 | 0 |
| BF65-38 | 44.44 | 0 | 1 |
| 8F65-39 | 28.13 | 0 | 0 |
| BF65c119-40 | 15.79 | 0 | 0 |
| BF65c238-41 | 21.05 | 0 | 0 |
| BF65c36-42 | 34.78 | 0 | 0 |
| BFES-43 | 33.00 | 0 | 0 |
| BF65c46-44 | 37.50 | 0 | 1 |
| BF65c7-45 | 38.10 | 0 | 0 |
| BF65c7-46 | 33.33 | 0 | 0 |
| BF65c9-47 | 0.00 | 0 | 0 |
| BF68-48 | 20.00 | 0 | 0 |
| BF68n2-49 | 16.67 | 0 | 0 |
| BF74c2-50 | 0.00 | 0 | 0 |
| BF75-51 | 40.00 | 0 | 2 |
| BF83/65c36-52 | 41.67 | 0 | 4 |
| BF85c2-53 | 45.45 | 0 | 2 |
| BF85c26-54 | 30.77 | 0 | 3 |
| BF85c51-55 | 45.45 | 0 | 4 |
| PGC-56 | 12.12 | 4 | 0 |
| PGC-57 | 12.2 | 2 | 0 |
| PGC-58 | 8.33 | 1 | 0 |

TABLE 1B
Competent Cell Lines:

| | Pregnancy | #Full term | #Ongoing |
|---|---|---|---|
| Cell Line ID | initiation (%) | calves | pregnancies (>90 days) |
| BF12n7-1 | 50.00 | 11 | |
| BF15-2 | 54.55 | 4 | |
| BF15-3 | 58.8 | 4 | |
| BF15-4 | 61.54 | 5 | |
| BF21-5 | 62.5 | 1 | |
| BF22/15-6 | 64.29 | 3 | |
| BF25-7 | 68.75 | 5 | |
| BF83/65-8 | 50.00 | — | 2 |
| BF84/65-9 | 68.42 | — | 7 |
| BF85c102-10 | 76.92 | — | 2 |
| BF85c19-11 | 50.00 | — | 2 |
| BF90/68-12 | 60.00 | — | 5 |
| BF91/65c42-1 | 58.33 | — | 1 |

TABLE 2
>‘000127a-001.scf’ came from CONTIG 1 at offset 0;
“E:\SEQUENCE\export\EST_db\000127a\000127a-001.scf” (43>314)
AATTTGGCACGAGACTCATTTACGAAACCGGGCTGGAACCGCAGCTGTCGCGGATCTGAACGCGAGCTGTTCCTGTGGGTGCGCAGCTACCTTGCTGCGGACA
GCTGAGGGAAAAAGGAAGATGGGGTCTTTTGTCTCATTTTTGAAACCTCGGAAAACTGTCACCATGCCCCACTCCTACCCCACCCTTTCTGTTGAGCAGAAAA
AAGAGATGTTCGACATTGCCCTGCGGGTTGTGGGGCCAAGCAAAAGCATTCTGGCCGCAATGAAAA
>‘000127a-002.scf’ came from CONTIG 2 at offset 0;
“E:\SEQUENCE\export\EST_db\000127a\000127a-002.scf” (48>536)
CTCGAGGTGGACACCACCCTCAAGAGCCTGAGCCAGCAGATCGAGAACATCCGGAGCCCTGAAGGCAGCCGCAAGAACCCCGCCCGCACCTGCCGTGACCTCA
AGATGTGCCACTCTGACTGGAAGAGCGGAGAATACTGGATTGACCCCAACCAAGGCTGCAACCTGGATGCCATTAAGGTCTTCTGCAACATGGAAACCGGTGA
GACCTGTGTATACCCCACTCAGCCCAGCGTGGCCCAAAAGAAACTGTATATCAACAAGAACCCCAAGGAAAAGAGCACGTCTGGGACGGCGAGAGCATGACCG
GCGGATTCCAAGTCGAGTATTGCGGCAGAGGTCCGATCCTGCCGATGTGGCCATCCAGCTGACTTTTCTGCGCCTGATGTGCACCGAGGCCTCTCAAATCATC
ACTTACACTGCAGTACACGTGGCCTACATGACCACAGACTGGCACTCAAGAGGCCCTGCTCTCAGGCTCACGAATNG
>‘000127a-003.scf’ came from CONTIG 3 at offset 0;
“E:\SEQUENCE\export\EST_db\000127a\000127a-003.scf” (55>553)
GCACGAGGCAACCTCACGCTGCGGGACTACAGCGGACAGGGAGTCGTGAAACTGGACGTCCAGCCGAAGTGCTTGGCCGTCGGCCCCGGGGGCTACACCGTGG
TCGTGTGCATTGGCCAGATCGTTCTGCTGAAGGACCAGAGGAAGTGCTTCAGCATCGACAACCCCGGCTATGAGCCCGAAGTAGTGGCCTGTCACCCGGGTGG
TGAGACAGTGGCCGTCGGGGCGCGGATGGAACGTCCGNCTCTACTCATCCTGGCACCACGCTGAAGACGAGGNCAGCTCTGGAGCCAAGGCCCCGGCCGACTG
GCGTTTCCACGACGCGCCTTCTGCTGGTGCGACCCACAAGTGTCACGTCTCACGTTGCGACGCTATCGAGACACGTTTTATGACACACAAGATGTTGCTGCCG
TCTCGACACACATTGCTGGTGCTGAATATGGATGGTGACCTGAGACTAACAGTANATCAATGCACGTCCATGACATGCTGTGAAAC
>‘000127a-004.scf’ came from CONTIG 4 at offset 0;
“E:\SEQUENCE\export\EST_db\000127a\000127a-004.scf” (48>649)
CCTCAGCTGCCCCCACCAGCTCCTGGGGTCCACCCACCAGCACCAGTGGTCCCCCAACCAGCCTCGGGGTGCATCCCCCTGCTCCTGGGGTTCATCCCCCAGC
TCCTGGGGTCCACCCCCCAGCACCAGTGGTTCACCCACCAGCATCTGGGGTCCACCCCCCAGCTCCAGGGGTCCACCCTCCAGCCCCAGGAGTCCACCCTCCT
GCTCCGGGAGTCCACCCTCCAGCCCCAGGGGTCCATCCTCCCCATCTGCTGGCGTTCACCCCCAGACACCAGTGGTGCACCCACCAGCTCCTGCAGTTCACCC
CCNAGTCGGGGGTGCACCCAACTCCTGCAGTTCACCCCCANGCTCCAGGGNTCCACCACCAGCTCCGGNGTCCACCCACCAGCCCCTGGNATCCACCCCAGCC
TCTGGNGTCACCCNNCTCTCTGNNGTCATCTCCGCTCTGGGTCCATCCAGCACTGNGTGCACCCTCAATCTGGTGCATTCCCACACCTGCCCGTGCTGAGCCC
CATACCTCGAGCCTGGACTTCTCCCTCCCACACTGAACACCCTACTCGTTGTTTGTTTTGGTTGCCTAAAAATTTTGTGCTGGG
>‘000127a-005.scf’ came from CONTIG 5 at offset 0;
“E:\SEQUENCE\export\EST_db\000127a\000127a-005.scf” (53>543)
GCACGAGGGTCATCAAGGTGCAGTTGGCAGTCCAGGCCCTGCAGGCCCCAGAGGACCTGTTGGACCTACGGGCCCCCTGGCAAGGACGGAGCAAGTGGACACC
CTGGTCCCATTGGACCACCGGGGCCCCGAGGTAACAGAGGTGAAAGAGGATCTGAGGGCTGGGGAGGCCACCCAGGACAACCAGGCCCTCCTGGACCTCCTGG
TGCCCCTGGTCCATGTTGTGGTGCTGGCGGNGTTGCTGCCATTGCTGGTGTTGGAGCCGAAAAAGCTGGTGGGTTTGCCCCATATTATGGAGATGAACCGATA
GATTTCAAATCACACCGATGAGATATGACCTCACTCAATCANGTCATGGACAAATAGAAGTCTCATTAGTCTGATGGTTTCCGTATAACCNTGCACGGACTGC
AGGNACCTGAATCTGCCATCTGACTCAGATGGAGATATGNNGTGTATCTACCAAGNTGCAAATGATGCTTTTAGTCTAT
>‘000127a-006.scf’ came from CONTIG 6 at offset 0;
“E:\SEQUENCE\export\EST_db\000127a\000127a-006.scf” (47>578)
TAAACCCAAGCCCTTGACCTCTTACAGGAGCTTTGTCTGCCCTCTTAATAACATCCGGCCTAACCATGTGATTTCACTTTAACTCAATGACCCTGCTAATAAT
TGGCCTAACAACAAATATACTAACAATATACCAATGATGACGACATGTTATCCGAGAAAGCACCTTCCAAGGGCACCATACCCCAGCTGTCCAAAAAGGCCTC
CGTTATGGAATAATTCTTTTTATTATCTCCGAAGTACTATTCTTTACNGNATTTTCTGAGCTTTCTACCACTCAAGCCTCGCCCCACCCCTGAACTANNGCGC
TGCTGACCCCCACGAGCATTCACCCACTAAACCCCTANAAAGTCCACTGCTCACACCTCTGTCCTATGGCTTTCGGAGTTCTATTTACTGAGCCATCATATTT
ATAGAAGGGACGAAGCTATATACAGCCTATTTTCACATACATAGATCTATCACACTATCANCTCAATATTGAGCACTTATTCTCGAGNGTTACGTCATTTTGT
ACCAGCTCCGCTCCTC
>‘000127a-007.scf’ came from CONTIG 7 at offset 0;
“E:\SEQUENCE\export\EST_db\000127a\000127a-007.scf” (52>397)
GCACGAGGAGATGGCGGGTGACGACAAGCACATGTTCAAGCAGTGGCTGCCAGGAGGGCCAGCCACATGTGCTGGAGCTCTGTCCACCCAACCTAGCCTACCC
ACAATGGTAAACCAGGGGGAGATGAAGCCCTACACAGGCACCGAGACCTTTACTATGCCCTATGATTTCGCGATCATTACCACCGACCTATTACCGACCACCT
ATGGGGACCATCATGGCGTTACGCGGAGATAGCCGACCCATGGAGGGAAGAATTGTGTGAGTATTAATCACAATGATAACTTGGAATGACTGTCTATATTTTA
AGGTAGTTGTTTTGGTTTAGTCCTATCTGGAGAGTGA
>‘000127a-008.scf’ came from CONTIG 8 at offset 0;
“E:\SEQUENCE\export\EST_db\000127a\000127a-008.scf” (46>662)
GGCCAGTCCTCTACCCTCACTTCAGGCTCTCAGATCCCAACTCTTGTCCCACTCGTCGAGCGTTAACTGATGTTATGGTTTAAGTAGTGCTGAAATTTTGGTG
GCTAATCTGCCTCCACCCCTAGCCACAGAGATCCCTTTTTTGGAGGAATGACTCGCGATGAAGACGAAGATGATGAGGAAGAGGAAGAAGAAGGAGTCACGTG
GGGCCGTGGGAACTCGAGGTTTGAGGGTCCCCAGTCCCCCGAGGAATTTAGCTTTGGCTTCAGCTTCAGCCCAAGAGGAGGAATGCGTTTCCACGATAACTTC
GNCTTTGATGACCTAATACGGNATTTCAATAACATCTTCAGCGAGATGGNGGCCTGGACCTTGCCTTCCCGCCCCTCTGTACTTCCAGGTCCTGAGTCAGAGA
CACCTGGTGAGAGACGGCAGTAGGACAGACGCTTCGGACTACTGCTTATTATNCANATATCACAGCCCAGACTTNGTGGGGCTGGNAAGGAGCANGACTGTAT
CTCCAACACACCGACGTGCCNCCAAACTTTCTAGNTGTGAACTGNCTGACCCCTCTAACAGAAAAATGATTTTTTCAGTTTCAGAGTTTGCCATTGGGCC
>‘000127a-010.scf’ came from CONTIG 9 at offset 0;
“E:\SEQUENCE\export\EST_db\000127a\000127a-010.scf” (52>598)
GCACGAGGGTGAGCAAGCCCACTCTGCTGGCATCACCTCCTGCTCAGGTTTTCTCTGCCAGCACAGCCCGGCTTTGGGTCAAGTGGTCAGAGTATGTGGCCAT
CTTGTCACCAGCGGAAGGAACGTGCAGGGAACATCGCTGCTCTGGTTGACTGACAGGCTGGGCTGCAGAGCGGCTCCTGGAGCAGCTCAAGGCCTGCGAGCTG
CAGGATGAGGCTGACATGCAGCGGTAGTTCCCGCATGGCCCAGGCCCTGGGCTGTGCAGGGGACTCNGATGAGGAGTTCACCAGCTGCTACCCACCACCCCGA
GACCTGCATCTTNGCACCATGACGATATCACAGGACAAGGCTGTGTTCATGGAGGGCCATGTTCTTGTGTCCACAGTGACCTGAGTCACTGAGGTTTATGCAC
AGACTNGTGGTCAATAACGGNTGTGTAGCCAGTCACTGGTGAGTANCCAACACGTGGTTTTCGAACAGGCAGCGTCAGGCTGAAGTGGAAGAATCAGATGTTA
CAGAACGATCACCCAACTCACGTGTTCTCT
>‘000127a-011.scf’ came from CONTIG 10 at offset 0;
“E:\SEQUENCE\export\EST_db\000127a\000127a-011.scf” (49>614)
GCACGAGGCTCCACAGTTCAAAAGACCCCTTTCATCACCAACCCTGGGTATGACACTGGAAACGGTATTCAGCTTCCCGGCACTTCTGGTCAGCAGCCCAGTC
TTGGGCAACAAATGATCTTTGAGGAGCATGGTTTTAGGCGAACCACACCGCCCACCACGGCCACCCCCGTAAGGCATAGGCCAAGACCGTATCCGCCGAATGT
AAATGAGGAGATCCAAATTGGTCATGTCCCCANGGGAGACGTAGACCATCATCTCTACCCTCACGTTGTGGGACTCAANTCCAATGCTTCTACNAGCCAAGAA
GCTCTCTCTCANACAACCATCTCATGGACCCCATTNCCAGAAACGTCTGAGTATATCATTTCATGTCATTCAGTTGGCATTGATGAGAACCTTACAGTTCGAG
TTCTGGATCTTGCTAGGCCACCTGACGGCCTCACAGAGGNCCACTACACATATATGAGGAGTAAAGACCACGAGCAGAAGTGCGGAGNTGGTACGTGGCATCT
GTGACAGGCGATCACCCAAGATCTGTTCACCTACGNTCCATTGCTGGAGAN
>‘000127a-012.scf’ came from CONTIG 11 at offset 0;
“E:\SEQUENCE\export\EST_db\000127a\000127a-012.scf” (48>603)
TTGCAAAGACACTAGGAAAACGGTCTTCTTTGCTAGGCTCTCAAGATGAGGCTTGGCCGAACAGGTTCTGTTACTATTTTTACCTTCACAGTTATCGTTAGTT
CCATATGTTTGTCAAAACACAGACCATTCTCGTTCCCCAGCTAGAAAGCAATAGGTTAAATTCTAAAAGCTGTTTGCTTTTTCGTCTTCGCCTTTAAATCCTT
GGAAGTTATCTCTTCCTGCTCCCCTACAGTATATATGGTTGGGAAACTGTGAAAGGAAGAAGGNGTGGTCTGTNAGGGAACTCCATCCATGGGGCCTNCTAGA
GCGGGTGTNGTGTTCCTACACCACTCCCCTCTCAGCAGAAGGGCTGCACACATACACCAGAGAACTTCGCCTTCCTTTTTCACTTICCACTTTCCCACCATGC
TTCGTTTACTACTAGATGAGAGTTATGACCACAGCTCTANACGTTACGCTTTGTATTATTAATTTCATACATANACATCTTGTCTACACCANACTACATAGTC
GTTGTCTATACTTTATATAGGNTAGNGGGATGTACTTATA
>‘000127a-013.scf’ came from CONTIG 12 at offset 0;
“E:\SEQUENCE\export\EST_db\000127a\000127a-013.scf” (52>585)
GCACGAGGCATGACTGACAATGATCTTATCAATATTCTTGACCCTTTTTATCATCTTTCAACTAAAAGTTTCAAAACACAACTTTTATCACAATCCAGAACTG
ACACCAACAAAAATATTAAAACAAAACACCCCTTGAGAAACAAAATGAACGAAAATTTATTTACCTCTTTTATTACCCCTGTTATTTTAGGTCTCCCTCTTTC
GTACCCTTATCGTACTATTCCCAAGCCTACTATTCCCAACATCAAACCGACTAGTAAGCAATCGCTTTGNTACCCTCCACAATGAATACTTCAACTTGTATCA
AAACAAAAATGAGTATCCACAATTCTAAAGGACAACATGAACATTATATTATATTCTGATNCTATTTTTTGATCATCAATCTACTAGCCTATACCCATTCTTT
CCACCACACACACTATCATAACTAGCTACCCTCCCTGTGGCGNAGCGGATCAGATCGCATATATAGCTACTGCCTTTTTACCAGACACCCTCCTATCATCTNA
TTGATTTACTTTTTTTCTN
>‘000127a-015.scf’ came from CONTIG 13 at offset 0;
“E:\SEQUENCE\export\EST_db\000127a\000127a-015.scf” (48>658)
AATAAAAAATACTATGACTAAAATTACTGTGGTTGAGAAATTTGGGTAATTTGGGTAATTTATAGTACCAGTGTTCAGAAATCTTCCTAACTTGTATTTGGTA
AAACCCAAGTAAACAATTTCAGCTAATCTCAGATGGATTATTTTACTTATGAAATTTCTCTGTTCACTTTCTCTTCTTTATCCTGCTTCACAGCTTTTGGGTG
TTGCCACATGATAAAGTATCATATCAACTCTAGATGATTAGGCAGAANATTTTTATCAAAGTCAAGNGTTACTTTTAGCAACTGGGTAAAGGNAAGNNGACTA
ATAGCTGCTTCATTANAAAGAAACTCAAAAATATAGAGNTCTCTTTANTAGACATAGCATACAGCATACAGTTCTTCACTAGATATTGCTTAGATGTTACCAC
TTCTTTGCACTATTCTAAAGATGAAACAGAANATAAACTACTANAACACAGGCAGAATCTCTCTATGTTTATTCTTTGCTCAAACTGGGCTTGATNAGGTTCT
ACTGAACCACTACCTTTTNTGTAGACAGGCAATTAACGGAGATTATAAGTCTTTTAGCCTTCCCACAAATTTTTAATGCTTTTTTCTACAGCATTAAAATTAA
TGT
>‘000127a-016.scf’ came from CONTIG 14 at offset 0;
“E:\SEQUENCE\export\EST_db\000127a\000127a-016.scf” (52>645)
GCACGAGGGTACAGAGGCCGGCTCCCGGAGCCAGCTGAGTTGCGGCCTCTACGACATGTCGGGGCACCAGCAGGGCACGGAGCTCGACCTTAGCTGGATCTCC
AAAATACAAGTGAATAAACCAGCAGTGCTAAGACGTGCAGAACAAATCCAGGCTCGTAGACCTGTGAAAAAGGAGTGGCAGGCTCGATGGCTCCTGAAGGCTG
TTACCTGTATAGATCTTACTACACTNTCAGGTGATGACACAGCTTCCAACATTCAAAGGCTGTGTTATAAAGCCAAGTATCCAATCCGGGAAGACCTCTTAAA
AGCTNTAAATATGCATGATAAAGGCATCACTACAGCTGCCGNTTGTGGTTATCCTGCCCGCGNGNGNGATGCAGTGAGAGCTCTAAGGCTGCGGCTGTGACAT
CCCATGGCATCAGGGGCCCTGGCTTCCGCTGCACAGACTCATTGATACACGATTAGAGAATAGATGGNCGGGNAGAGGGCTACGGAATGAGGGNGGATAACAG
ACTGGGCTGCAGCCAGGAAGCCGNTGAGAGACGCAGTGCAGGCGGGGAGCATCCAACATCTACCAGAATGATCTCCAAN
>‘000127a-017.scf’ came from CONTIG 15 at offset 0;
“E:\SEQUENCE\export\EST_db\000127a\000127a-017.scf” (52>584)
GCACGAGGAAATTATTTACTAAACTAAACCTTAGGAAAAGATGTTTTGCATCAGTCTTAATAGGCAAAAGTTCATGCTTAGTGACTGGAGAGATTTTTGCTTA
TAGCTTTTCCCTCTATAAATACTATCTAGAATGAAGCTAATTTAGGAACAAGACTAACATTCAAAAAACCCTTAGTGCATATATTTTTAATTATTATTAGCTC
ATTATTAGATCATTTATTNTCATCTGTATTTGCCATTAAAATTTATTTGCCTTTATATCTTTAGAATATTGAGTTTGTGTCTTTCTGTTTATCATTGCTACAA
GTTTTATAAAAAGAACCTTCACTAGTACATGCCAGAAGATCATATTTCTGCTAAGTATTATTNTTTTTAAAATCAGATGTCGCTGTATTATTGGTCATGCAGC
GAGTAGAGGAAATGTACAGACAGAGTTTTCTTTAGCCATGACCTTTCTCTCTGNAGNTGNTGCTTTAAGCAGATTTCTCTTATATATGNCTTCTTTCTCTACT
CTTAGATACTTGCTGT
>‘000127a-018.scf’ came from CONTIG 16 at offset 0;
“E:\SEQUENCE\export\EST_db\000127a\000127a-018.scf” (44>670)
GAGCCCCACCTGGAGCTGCGCGCAGGCTTGTAGCGCGGAGCCCAGCGCAGAACCTCGCCCGCGCAGTCCCTCGCGCGCCCCGCGTCTCGGCGCTGATTTCCAG
GCCCCGGAGCCGCGCCAAGCGCTGCGAGCGGACCCGGGAAGAGCTCCGGCCCCCGCCGCCACCGCTTCACCGGCTTGGCTCCCTCCGCCCCCGGGGGGGTCGC
GCACCCACGATGCTGCAGGGCCCCGGCTCCCTGCTGCTGATCGTCCTCGCCTCGCACTGCTGCTTGGGCTCGGCGCGCGGGCTCTTCTTCGCCAGTCCGACTT
CCCCTACAAACGCAGCAACTGCAAGCCCATCCCGGCCAACCTGCAGCTGTGCCACGGCATACAATATCAAAACATGCGGCTGCCCNACCTGCTGGGNCACGAG
TACATGAGGNAGGTGCTAGAGCAGGCGGCGCCTGGATCCGCTGGTCTTGAAGCATGCCACCGGACACCAGAAGTTCTGTGCTGCTCTTCGGCCGTCTGGCTCG
ACGACTGGAGAAACATAGCCGGCACTGCTCTGGTGCAGTGAGACCTGGCTCATCATGTCGCCTTGCTTCGTGGNCGATGCTGATGTACGCTCCCAGAACACAC
TTGCTCCCTCT
>‘000127a-019.scf’ came from CONTIG 17 at offset 0;
“E:\SEQUENCE\export\EST_db\000127a\000127a-019.scf” (51>444)
GCACGAGGAAGAGGCCTCGGGCCCCACGAGCATCCGCGTGCACTTGGAAGCCGGCCGCTTCCACCTGGACGGCAGCCGCGAGAGCTTCGACTGCCTCTTCGAG
CTGCTGGAGCACTACGTGGCGGCGCCGCGCCGCATGCTGGGGGCCCCGCTGCGCCAGCGCCGCGTGCGGCCGCTGCAGGAGCTGTGCCGCCAGCGATCGTGGC
CACCGTGGGCCGCGAGAAACCTGCGCGCATCCCCCTCAACCCCGTCCTCCGCGACTACCTGAGCTCCTTCCCCTCCANATCTGACCAGCCGCACACCGCAACA
TTACTGNAGCGCCCTCTACTATTTTCTATATTATTATTATTTTNNCTGACATGTGGNTGCCTTCCCATCTGGTGTAGGTACGG
>‘000127a-020.scf’ came from CONTIG 18 at offset 0;
“E:\SEQUENCE\export\EST_db\000127a\000127a-020.scf” (41>577)
CTCGTATTTGATGGACTCTAACTACATCGATCCGAAATTTCCTCCATGCGAAGAATATTCGCAAAATAGCTACATCCCTGAACACAGTCCGGAATATTACGGC
CGGACCAGGGAATCGGGATTCCAGCATCACCACCAGGAGCTGTACCCACCACCGCCTCCGCGCCCTATCTACCCTGAGCGCCAGTATAGCTGCACCAGTCTCC
AGGGGCCGGGCAATTCGCGAGGCCACGGGCCGGCCAGGCGGGCCACCACCACCCCGAGAAATCACAGCGCTCTGCGAGCCGGCGCCTCTCTCAAGCGCCTCCG
CCTCCCCGTCCCCAGCCCCGCCAGCCTGCAGCCAGCCAGCCCCTGACCATCCCTCCAGCGCCGCCAGCAAGCATCCATAGTCTACCATGGGATGAAAAAATCC
ACGTTGCACGGTGTACCCCATTTACGTTAGGGGAACCNACGCTCGAGACGCTAACCGCAGCAGTCTGATAAAAGAAGAGATTATTACATCGTACTGACGAGAG
AGAGGAGATGGCCATGTGG
>‘000127a-021.scf’ came from CONTIG 19 at offset 0;
“E:\SEQUENCE\export\EST_db\000127a\000127a-021.scf” (38>612)
CTGCAGTTGGCCCCTACTGGCCGGGGTGGTCTGGGGAGTGGACAGATTGAGATTGTGTTGGCAAGTGTTTGTCTTTTCTGAATGCAATGTGGCAAAGCAGGAA
AAGAGCCTAGGTTTGCAGAATATATGTGCGTGCATGCTGAGTTGCTTCAGTTGTGTGACCCCACGGACTGCAGCCCACCAGCCCCCTCTCTGGCATTAATTCT
ACAGGCAGGAATACTGGAGTGGGTTGCCTTGCCCTCCTCCAGGGATTTCCTGACCCAGGATCAAATTCACGTCTCTTACATCTCCTGCACTGGCAAGACAGTT
CTTTACCACTGCCCCACCTGGAAGCNAATATACACANGATGACAAAGCTCAAACTCATTCTGACCCCACACCTCTGTCTGTTCTAGTCCCACACGAGCTTGCT
CTTTCTACTGNGNGNCCACTAAACGACTGTTCTNCTGRGCGCTTACTCACAGTATGGNNCTATCANAGTCACTGTTGCTTTGATGCTAGTCACATAACCTGGA
CTTCTACCTTTTATGTTTGTTTTTTATAAAAACTAACTGATTTTTTTTTTTTNTTATC
>‘000127a-065.scf’ came from CONTIG 19 at offset 0;
“E:\SEQUENCE\export\EST_db\000127a\000127a-065.scf” (46>576)
TTTTTTAACTTTTTTTTTTTTTTTTTACTGGCATTTTTGTCTCTGATTCTCTTCAGCCCTCACCCCTGGCCTTCATCTGTCTTGATTGACATCTTTGCTTTCT
TCTGTCCCCTTCACTCCAGATCCCTAAGTTCCCTTCCAGCTTGGGGACTCAGGGTGGGATGTGGTGTGGAGGAGAAGCCCCAGGCCCAAAATTCCATCTATTC
TTCCTGGATCCCAGAGGGTGGGGTAGAGAAGAGGGGGGNCATCCCCAGCCCCCCAGCACTGAGGAAGAATGGGGCTCTTAAGGCCTTAGCTCTGATCCCTTCC
CCCTTCTCCCTGCCCCCAGNACTGNGCCACTTCTGAGTTGGGCAGCGGGTTCTAGCTCAGCTCAGCTGAGAATGTTAGAAACTACAACATAATTCTATAATTA
GTTTTGTGTCTTCAAAAAAAAAAAAAAAAAAACTGAGGGGGCCCCCGTACCCATCGCCTATGGATCGTATACATTCCGGCGNCGTTACAGCCGGACTGAAAAC
TGCCGTACC
>‘000127a-022.scf’ came from CONTIG 20 at offset 0;
“E:\SEQUENCE\export\EST_db\000127a\000127a-022.scf” (48>664)
TTCTCCTTTGCCTGGCCGGGAGGGCCTTGGCAGCCCCTCAACAGGAAGCCTTGCCTGATGAGACAGAAGTGGTGGAAGAAACCGTGGCCGAGGTGGCCGAGGT
ACCCGTGGGAGCCAACCCCGTCCAGGTGGAAGTAGGAGAATTCGATGATGGTGCTGAGGAAACCGAAGAGGAGGTGGTGGCCGAGAACCCCTGCCAGAACCAC
CACTGCAAACACGGCAAGGTGTGCGAACTGGACGAGAACAACACCCCCATGTGTGTGCAGGACCCACCAGCTGCCCTGCCCCCATCGGCGAGTTTGAGAAGGT
GTGCAGCAACGACAACAAGACCTTCGACTCTTTCTGCCACTTCTTTGCCACCAAGTGCACACTGGAGGGCACCATGAAGGGGCACATACTCCTACTGGACTAC
ATCGGGCCTTTGCAATACATCCCNCCTGCCTGGACTCGANTGATGTATTCCCTTGGCATGCGGATGGCTAGTACGTCTGTCAGCTGTACGAAGGAGAGGACAC
ACTCTGACGATACAGAGCTGGAGAAAGATCACGAATGAACGCCGGGCGGGACATCTGGATGCGNCGGATGNAAAATAACAGACTTTCTGCCGGAACGG
>‘000127a-023.scf’ came from CONTIG 21 at offset 0;
“E:\SEQUENCE\export\EST_db\000127a\000127a-023.scf” (45>583)
CTGGGGGAAGACCATGGACTTCGTCGACGTGAATGAGAGCAACGCACGCTGGGTGCAGGATCTTCCGCCTCAAAGCTTACGCCAGCCCCCGCCAAGCTGGAGT
CCATTGACGGTGCCCGCTACCACGCCCTGCTGATCCCCAGCTGTCCCGGGGCCCTGGTGGACCTGGCCAGCGGGTCCCTGGCTCGCATCTTGCAGCACTTCCA
CTCTGAGAGCAAACCCATCTGCGCTGTGGGCCACGGCGTGGCCGCCCTCTGCTGCGCCACTAGTGAGGACAGGGTCTGGGTGTTCCAAGGCTACAGCGTCACC
GGGCCCTCAGTGTACGAGCTCGTGCGGGCGCCCGGTTTCGCCCACCTGCCCCNTGATGTGGAGACTTCGTGTAGGACGCGGGGNCCTGCTTCAGCGCCAGCGA
CCTGACGCATGCATGTGTGCTGGATCGCCACCTGGTCACGGNCAGACGCCAGCTCACATTCTGNCGTGCAAANCTGTCTTNCTTGTGCAGCGNAAGGAGNAGN
CCCNGGCAGCCCCGACAATGCATGHCGRGCAAANCTGTGTTNCTTTGTGCAGCGNAAGGAGNAGNCCCNGGCAGCCCCGACAATGCA
>‘000127a-024.scf’ came from CONTIG 22 at offset 0;
“E:\SEQUENCE\export\EST_db\000127a\000127a-024.scf” (54>633)
GCACGAGGGAGGTCACCCACATCCCTTCAAGATGAAAGCCGGGTGCTGACCTTGGCTGTGCTCTTCCTGACGGGGAGCCAGGCTCGGCATTTCTGGCAGCAAG
ATGACCCCCAGTCATCCTGGGATCGGGTGAAGGATTTTGCCACCGTATGTGGAAGCAATCAAGGATAGTGGCAGAGACTATGTGGCCCAATTCGAAGCCTCCG
CTTTGGGAAAACAGCTCAACCTGAAACTCCTGGACAACTGNGAACACCCTGGCCAGCACGTTGTCCAAAGTGCGTGAACAGCTNGGCCCAGTGACCCCAGAGN
NTCTGGACAAGGTGNAAAAAGAGACCGCGTGCCTGAAGCAGGAGATGCACAAGGACCTGAGGAGGTGAGCAGAAGTGCAGCCCTACTGTACGAGTTCAAAAGA
GGGCAGAGGAGTGGGAGACTACGNCAGAGTGGCGCTGCGNNAGAGTCGGAGGGCGGGCAAGTGCAGAGTGAGACAGTGACCGTGCCAGACTCGCACGCGCCGC
CCGAAACTGGCAGAGTGCGCTACGACACTGCAGGTGCGCCGCGGGGGCTAAGGGGGGGCGGGA
>‘000127a-025.scf’ came from CONTIG 23 at offset 0;
“E:\SEQUENCE\export\EST_db\000127a\000127a-025.scf” (47>576)
TTTTTTTTTTCCAGTTGGATAAATCATGAGATTTATTTATATTTCACTTTAAAAGCCACAACATAACAAACGTCACTTTCTGGAGAGTCATCTTATTATAAAT
ACATCAAGTGCACTTAATACAATGAAACTCGTCTTTTGGTACCACTGTGAGACCATCAGGTGAGGCTTTGTTAAATTTCCCTCTAAACTTAGGCTGAGATGAT
CTCAATTCAGGAGGGTCATAGCAAACTTTTTCCAAAACTACAGCGAAGAAGCTGTAACAACACCCGCGCCCTCCCAATGACAACGCNGTAGAGAAAGTTACTT
TGCCCCACTNACCCGGGCCCTACCCGCTCTGGCACGTGAGATTCAGCAGCAGGAGACGAGCGCCACAGCGTGTGCAGGCCAGACCCGGTCACTGTACTCAGNC
CTGACCACAAATATCCTATCAACGAAGTCTAAGGTAAAGAACTAACAGGGGCTCTGTCTACAAGCTTGCTCTCAATGAAACCCGNCTTTATAACTGGTACAAG
GACTCACTTGGTGTT
>‘000127a-028.scf’ came from CONTIG 24 at offset 0;
“E:\SEQUENCE\export\EST_db\000127a\000127a-028.scf” (53>645)
GCACGAGGGGAGGTTGGACGCTGTGCACCCTCCTCCCCTCCTCTCGCCGGCAACGTCTGAATCCGGACACCATGGACTCGGTTCGCCCCCRCRGGCTGATGCT
TCTGTCGCTGCTCCTCGTGGGAACTGCGCTGGGTGATGCCTCGCAGGCGCCGCCAGGAAATAACGCGGAGATCTGCCTCCTGCCCCCGGAGGACGGGCCCTGC
CGGGCGCGGATCCCCAGTTACTACTACGACAGGTACACGCAGAGCTGCCGCGAGTTCATGTACGGAGGCTGCGAGGGCAATGCCAACAATTTTGAAACTTTGG
AGGGCCTGCACGAAGCGNGCTGGAAGATTGAGAAAGTTTCCCAAAATTGCCGGNTAAAAGNGAATAGTAGCAGTGTGGGGAGCTCAGAGACAGTATTCTTCAT
CTAGTTCTTGACATGTAATAGTTATATTGCGGTGTCACACATGAGACCGGTCCCGTGAGCTACTGTATGACTCTGGCACCAAGAGACTCATATTTGCTACACC
CAAGAGAGGCTGTGTTGCTAGTCCTCGTATATTTTACCANAACAAGCTGAGGCTCACTACGTGNGAGGAGNATAATTGC
>‘000127a-029.scf’ came from CONTIG 25 at offset 0;
“E:\SEQUENCE\export\EST_db\000127a\000127a-029.scf” (49>593)
GCACGAGGCAATACACTAGGTAATACTGTTTTAGGTTTCCCTTTTTCCCTCTTTATCTGAAAGTATCCTTGGTGTACGGCGTCGTGTCAGCTTCAGAATACTG
GGTGCTCGTCACCTGTACACACGCACAACCTGTGGTTTCTCAGGGTCTTCAGCGGGATAGCTTGGGGCACGTGTTCACTGGCGTTCTCTCGCTGCCTACGGTG
GGTTCTTGTTGATTAGAGAATCTAATAGTTTATGTAGTTGAATCCCAACCTTTTCAGGTACCCGCCCACTGTATTGTACTATACTTCCTTTCGTTTTCTTTCT
CCCTCACTGAGCCTGTTCTCTTTGAAGRGCACATTTCACACTATTTAGATCACACATGTTACGAATATCACGTAATATTGTCTGTCTGTAGTTGTATTAGATG
GTTCGTGGGATCCTCTGGGTCCATTCACTAGCTGCATGTACATTTTTCTTGTTNCATGNTGAGTATGTATTCATATATTGTTCACGATTTTACAGTTATTGTC
TTGTATTTTTATTTTCTGTTATATGATG
>‘000127a-030.scf’ came from CONTIG 26 at offset 0;
“E:\SEQUENCE\export\EST_db\000127a\000127a-030.scf” (53>583)
GCACGAGGATGGGAGTAGACATCCGCCACAACAAGGACCAAAGGGTCGACGACAGGAGCCCAAGAGCCAGGACATTTACCTGAGGCTGTTGGGCAAGCTGTAT
AGGGTCCTGGGAACAGCGAACCAACTCCACCTTCAATCAGGTTGCCCTCAAGAGATTGTTCATGAGCCGGACCAACAGGCCACCGCTCTCTTTCCCGGATGAT
CCGGAAGATGAAGATGCCTGGCCGGGAGGGCAAAACAGCTGTGGGCGGGGGACTATAACCGAAGATGTTCGTGTGCAGGAGGTGCCGCAACTGAAGGTGTNGT
GCTTGCGAGAGCAGACGCGCCCGCAGTACTCGCTCAAGGCGGNAGTGAGCGGCAAGCGCGAGCCTCGGCTCATACT
>‘000127a-031.scf’ came from CONTIG 27 at offset 0;
“E:\SEQUENCE\export\EST_db\000127a\000127a-031.scf” (47>393)
CrrCGrrCTGAATGrfGAGCThfAAGCCAACThFFrCCCTCTCGTCCACmCATCIGAGGCmYrAG117CCTCnCACrFI7CTGCCATAAGGGAGGNGATCATC
TGCATATATGAGGAUGATAmCTCCTGGCAATCUGATTCCAGCTIGTGC2FFCIFrCCAGCCCAGCGGUCTCATGATGTACTCTGCATATGATAAATAAACAGG
GNGACAATATACAGCcTGACGTACTCCTTCCTATGGCCACGCTGGCCATGTCCAGGTCnACTGGCGTCCTGACCTGCATACACGAflCTCGAGCTCGGGNGCC
GG
>‘000127a-032.scf’ came from CONTIG 28 at offset 0;
“E:\SEQUENCE\export\EST_db\000127a\000127a-032.scf” (53>635)
GCACGAGGCnPCTCCAATCATAAACCACGTGGCCAGGGGCCGCTGCAGCCACACAGCCGGACAGCCTCTCAATACTTI7GTGCTCTFJTGATCKFI0ACTGAT
GGCGTGATCACAGACCTCGATGAGACCAGACAGGCGAYI7GTTAATGCTGCCAAGCTGCCTATGTCCATCATCATCGTCGGCGTTGGAGGCGCGGACflCGGC
GCCATGGAGCCTAGATGGCGACGGCGGGCCTCCGCTCCCCGACCGGCGGGCCGCCAGGGATAUGTNCAGAUGTGCCTCAGGCAGCTCCAGAACGCTCCAAAAG
AGCACTGCTCIAGCGNCCTGGCGGNAGGCCCCCAGCAAGGGANGGGGCTACnCATCACATACACTCCmCTCCCCAG1CCCGGCCGAAATGAGAGCGCCCTGGT
CGTCGAGCAGAGTGTGCTGGTGGAGCACAGACTCACATCTCAATGCGArrGTCACACCTCTCANCCTACATTACATGCACCTACGnGGAGTCATTAAGCTFIT
CrFrTACTG1YrGAGAAGGCTACYFAGTTGC
>‘000127a-033.scf’ came from CONTIG 29 at offset 0;
“E:\SEQUENCE\export\EST_db\000127a\000127a-033.scf” (48>605)
CTAGTGGrrArrAAGACmCGGGGCTGAffAACCCTAACTCTCCCnCAGGGCAGCCCIGTGCTAGAAGACAAGGGTG71TGGC7FITCGGGATGGTGGACTCTG
AGAAGGACGCGGCTGTAGCCGAAGCTAGGACTGACTGAAGAGGACAGCG1YIATGT1TTCAAGGGGGATGAAGTCAGAGTACGATGGCGAGTrrCTGCTGACA
CCCTGGTGGAGTCTGCYfGATGTCCTAGAGGACCCTGTGGAAGAGGAGGTGAACGAGAGCTGCAGGCAPGAGAATAUGAAGATGATAACAANACTA1TGGCTA
CCGCGACTCANAACAYFACAAAGCCTNATGAGACGCCGCGGNAGATGCACCCCTACAThCCThCTCCCCTNCGACGCAAGTGGCAAAGTAGCTGACCCTAAGC
TGATGAArrGKFICTACGAGCCTCATGGNAGANCTGTGACATCCCAACAGCCCACAGCGAAGAAACGCAGCTCGTNAGCACCAGAAACAACTGAGAGTGAGCT
GAATIIGATGT
>‘OO0127a-034.scf’ came from CONTIG 30 at offset 0;
“E:\SEQUENCE\export\EST_db\000127a\000127a-034.scf” (47>553)
CTGCTTGGCAGTTTGATTTTTAAATTGTGTTAGAACATAAGCTGTTTCAGAAAAATATGAAAAATGTATGGCTGCCTTTTGAAATATTTGATGCCTTGTCCTA
CAGGATACTGCAAAGAACATGGCTGTCCTAAAATTGTAAAAATTGTATAAACAAGTCACAAATGCCAGTTTTCTAAAAACTTTTCAGATTTTTCCCTTGATAT
GAAGGTAAGGAACATATACAGGTATGGAGTATTTGACTGAAAACAGTGTAGGTTATGGTGGAGACACAGACACAGAATTTTCAGAGATTTGCTAGTGGTAGGT
ACTGAANTGAATACCCNNAGTAGCTGTAATGTCCCCTGAGACAGGTAGTCTTTCATACTAACACAGAGACTTTGTTGGNTCATTATAACACATGCGATGTNGT
AAATGTGNTCAGGGAGAAGNTAGGAACTTGNATGATTTGGACAAGAGTTGAAAGGATATCATAGNTAGAGGAGGNTGAAANTACTGNAAGTTGNT
>‘000127a-035.scf’ came from CONTIG 31 at offset 0;
“E:\SEQUENCE\export\EST_db\000127a\000127a-035scf” (48>603)
CAGGTGGCACAGCCCGCCATCACCGACAACAAGGATGGCACTGTGACTGTGCGCTACGCCCCCAGTGAAGCCGGCCTGCACGAGATGGATATCCGCTATGACA
ACATGCACATCCCAGGCAGCCCCCTACAGTJCTACGTGGAIFITATGTCAACTGTGGCCATGTCACAGCCTATGGGGCAGGCCTCACCCATGGGGTGGTGAAT
AAGCCCGGTATGTICACCGTCAACACCAAGGATGCGGGCGAGGGGGGC1TGTCCCTGGCCATFIGNAGGCCCCTNCAAGCAGAGATCAGCTGCACCGACAACC
ANGATGNGACGNGCAGNNGTCTCCTACTNGCCGTGACCTGGNGACTACACATCCTGGNCAAGTACAACGACAGCATANCCGGGCAGCCC2ITCACTGNCAGGT
CACAGNTGACGACTCCTGCGCATGTCCACTGAAGTGGGCTTGNCGNCGACTCCGATCACATGCGNAGACGACTCACCTCTGACGCAAAGGNGCCCCCTCGGCG
GNAAANCTGCTGTGNANCGGTGGCAGNCACAGGGATCTATCTC
>‘000127a-036.scf’ came from CONTIG 32 at offset 0;
“E:\SEQUENCE\export\EST_db\000127a\000127a-03 6scf” (48>605)
CAAAATCCTCACCCTGCAAATGAGAATGCTGGGTGGGCACAGAAGCACGTGAGAGAAGGCAGGNGTGCGGXAGCCCACCTGGGGGCTGGGCTCCCCAGTCTGC
GCACCfCAGGCTGCTTAACTCAAGCTTGAGTTFGTGGACCTGCTCAGTCTGCTCGACCACAACCTTTCATAGGTGAflGCTAAGAGGG1TITITCflAAAAAG
AAAAAAAAAAAAAGAATATTGTCAAAAATGGflG7ITTGCACACCCTGTGAATT7FfC1TCCTCCAAATGGAGACTCATGT-ErATGACTACTAT-rTAAAAA
GACTCCAYrTAAAGCACANTFrTATGAAAACAAATAANTCCATGTFrTAATGTCATGTATACTFFAATATCPTCTCTACAGTAGCTCAGTTATAGAGTGlTrl
TrATTACAA1ITATGIIT1TGTCGGAGGAAACCGCCCAGAGAGNGATCGGGACAGGAGAGNTATGITrGrrCTAAnATFrGAGTGGGcTATAC7FrCGCTGGG
TTCATGTTC1TGGTGACTACAATAATrCIFrCAACTAAAAAATCAYITAAG
>‘000127a-037.scf’ came from CONTIG 33 at offset O;
“E:\SEQUENCE\export\EST_db\000127a\000127a-037scf” (46>513)
CTGACGCTGAGCAGCGTGGTGAGATGGCTGTAAGGATGCTCATGCCAAGCTGGCCGAGCTGGAGGCCGCTCTGAGGAACGCCAAGCAGGACATGGCGCGGCAG
CTGCGCGAGTACCAGGAGCTCATGAATGTGAAGCTGGCCCTGGGACGTGGAGATJGCCACCTACAGGAAGCTGCTGGAGGGCGAGGAGAGCCGGCTGGAGTCT
GGGATGCAGAACATGAGTATCCACACCAAGACCACGAGAGGCTACGCAGGTGGACTGAC2FI7CGTCCTACGGGACCCCTGGCrrCAACTACAGCCTGAGCCC
CGGCTCCTTCAGCCCACCAGATCCAAGCCTGTGGGTGTGAAGAAGAATGAGACCCGCGATAGGAAGACTGGGGTCGTGTTCTCTGATGTGCTGTGCTAGTGTA
TGGGCTCTGCGGNGCCTrCCATCCTCrI7CGCITCATGCTTCTTGCGAGNAGCTGGCG
>‘000127a-038.scf’ came from CONTIG 34 at offset 0;
“E:\SEQUENCE\export\EST_db\000127a\000127a-038scf” (1>498)
AGGGCGTTAATAGTCCGCAGATrGCAGTCAAACACCCGCTCACGACCGACCCTGTCTCCGCTCGCGCCCATCTACACAAGGCCCCGTGGAACAAGGITCGCAG
GCTGGGGGTCCCCCCTCCTGAAGGACAACGTCTCCTACACGGGCAGGCC7FPTGGTGCTGTATCACTGACTGAGAGCAGAGCCTCCTGAGAGCCTGAGCTCGT
CCTGCACTCCCACCCCATCCCNACCAGGCGGCCCGGCTCCTCCAGTGCAGATGGCACAGGGGGTGGACAGCTCTCCTCCAGTGCCCGGGACCTGCACCCACCA
CGNNGCTGGAGCTGGGGCAGATGGNGACAGCGACCCTGCGCACTGCAGGGATCTACGACTGTNCTGGGCTCAGGCTGGGCAGTGGCGITIGTNCAAGNATATT
ATAATCAGCTGTGCTCCCAAAAAAAAAAAAAAATAAAAAAANAAAAAAAAAAAAAAATGAGG
GGGCCGTACCCATCCCTAAGGTGTA
>‘000127a-039,scf came from CONTIG 35 at offset 0;
“E:\SEQUENCE\export\EST_db\000127a\000127a-039scf” (54>514)
GCACGAGGGAAACAGTGAAAATAGCTCTGCTGTGTGCTGGAGCTCAGGAG1TFJGCCAAAACAATCTGGGCCTACATAAGATYGGAAKFATCTGTCTATGGTG
AGACCTGAAAGGAGTGAT1TAGACAAGAAACAATGTCCATFCAGCAATATFJCCCAAAAGGAAACUCACCCCYI0CAAATGGTATATGGAAACTGYrGCTATE
FI7CCTAAAAT1TEFAAAAITITrTCTAAATGACTGAGTGCTAAATACTGYIACTCAAGThJTAAATGCCACCACTCAAGGAAAGAGAAACTATNGAAGAAAT
AArFA7m7AATATANTTGCAGTFI7GGGGGAGAAGAAATAATACAThrAGNGTATTAA7FrCATATGCTAGGAAGTGCATCTAGAATTFATGGGATG1TrGAT
GGNAGAGTTGTGCTGGTACTGAAGATACAACTTTNThTGTflTATITGGNGTA
>‘000127a-040.scf’ came from CONTIG 36 at offset 0;
“E:\SEQUENCE\export\EST_db\000127a\000127a-040.scf0(53>617)
GCACGAGGACGGCAGTCGCTAGCCCCTCACCACCTCCATCCTGACTCCTGACCTGTAGG1TGGGCACCACCATCAGAGCCACCTCCCAGTGCTGACCCCTCCC
CTCAGCAGCCCTGTAACAAGTGCCHGTAAGAAAAGCGGGGGAAGTGGGAGCAGCCACGYITAGTCTCTGGAGGTAGGTTATCCCTGGGAGACYFGAAGGCTGG
G1TFGArFAAGAAAACTCTPTCCACCCCCCACAACTACTrCCGGACTAAGGAAYFAGGGGAGCATCCGrrCAGAAGCCTGAGAAG7FrATCCTATGCTGATGG
AGGAGCCATGCTGCTTCATCCTGCGTGAATGCAGNTGGCTCTCCITGCTGCTGNGATCACCCCAGCAGACCCATAGCCCCCCAGCCTGGTGCTGGCTGCTCCA
GCCCACCATGGTACATGGCTCCCCATACATAGCTCAflCCCANCATGISINAGAAGCCNAGTGCGNAGNTCTGNGTATGTNATCACCAGCCrrGNCTGCrFCG
GGCTCACAGCACGGAGGAACACCCGCTTCTCCACCTACYIGIFI7GATCTAAAAGA
>‘0001 27a-04 1 .scf’ came from CONTIG 37 at offset 0;
“E:\SEQUENCE\export\EST_db\000127a\000127a-041scf” (52>584)
GGACGAGGGCAGCAGCTGGGGGCCGGCAGflGCCYI7GGGGACCGCGGGCCCCTCCCTCCTCCTCTCCCCTGCGCCTCGCGTACCCCACCGAGGCGCGGCCGA
CTCCCCGGCCTCCCTGCCGCCGGCTCGGCGGAGCCGCAGCGGCGCCCCGCGAGAGGCGGAGCCGCTCCCAAGATGTCGCAGACGGCCATGTCCGAGACCTACG
AT7TflTGTfTAAKFCYTGGTFATGGAAATGCAGGAACTGGCAAGTCITGCTTGC7FJ0CATCAGThTATFITGAAAAAAAAATCANAGATGACTCANATCAT
ACAATAGGAGTGGAATNTGGTTCAAAGATATAAATGNTGGNGGThAATATGTAAAGNTACAGATATGGGACACAGCAGGCCAGAACGATTCAGGTCTGTGACA
AGAGCTACTACGAGTGCAGCGGGGCCTGCTGTCTACACAYITACCAGCGAAAACTACATGCGCTACTAATGNTTACAGATGCCGATGCTGCGAGCCAAACACG
NCTCATCCCTGCGGAACACAAG
>‘10001 27a-042.scf’ came from CONTIG 38 at offset 0;
“E:\SEQUENCE\export\EST_db\0001 27a\000127a-042scf” (48>516)
TFTGAGAAACCTCTGCGCCATGAGAGCGAAGTGGAGGAAGAAGCGAATGCGCAGGCTGAAGCGCAAAAGAAGAAAGATGAGGCAGAGGTCCAAGTAAAC7FGT
ACACCCATGGAAGCCACAGAAGCAGAAACAAGGGAAGCCAGAGGCCAGGGACGCTGGTACAAAGTGTGGACTGCATGCCTACTATCTAGAACTTATCAATGGA
TCTGGAACATCTATGGCCATTCTGATCACCTITGACCACCTTTGCGAGACCTACCTTGCTCATATCAAAGCCGTCCCT1TTGGTCCATTGCCCTGGACCTGTG
ATAACTATGGACTAGYfCTCTCTCAGTFFGTGGCTGAATGTAACGNGTACAATAAATCATCTNCTJTGCTGTCTTATCGGAAGAAAAAAAAAAAAAAAAAAAA
AAAAAAAAAAAAAAAAAAAAAAAAAAAAAACTGAGGGGGCCGGNACCNATCGNCCTATG
>‘1000 127a-043.scf’ came from CONTIG 39 at offset 0;
“E:\SEQUENCE\export\EST_db\000127a\000127a-043 scf” (52>491)
GCACGAGGCCCCACTCTCCCCCGCCCCGCTCTITPITFAAACATGTGATEITGTGTATFrGGCCTGAGCTGAGCC7FICATCGCAGTGTGTGGGCTIGCTCTA
ArrGCAGCTCTCAGTC1TCTCTTGTGGAGCACGGGCTCCGGAGCGCATGGACTCAGTAGFI7GCAGCTCGAAACTCYFG2FFGTCACAAGCTGGC7F17AA1F
ACCATGTGCCATGTGGAATGTTAGCTCCCCACCACGGGTCTAACCCGCGCCTGCG2FrGGAAGGCAGA7IITCrrAACCATITGGCCACCATCGCAATATCAN
GGCCTGCCTCTGCTAACCACACTCCATACATCCCrTCTTNCTCCGCTCCNNCCTGCACGTATCTGTCTCTrGTCTGGATGCTG1TTCTAGTAGTTCGThIGGC
CTGTGTrIITCCATCTCYI7ATGrFGnG1TTGCTCNNTC
>‘000127a-044.scf’ came from CONTIG 40 at offset 0;
“E:\SEQUENCE\export\EST_db\000127a\000127a-044scf” (50>491)
GCACGAGGAGGCCTCGCTCTPCCTCGCGAACAAGCATCATGAGCTTCAACACCCAATCCACCTTCTCCAACTACCGGTCCCTGGGCTCCGTGCAGTCGTCGGG
CCACCGGGTCCGACCGGTCAGCAGCGCGGCCAGCGTCTATGCAGGCGCCGGGGGCTCGGGCTCCCGGATCTCCGTGTCCCGCACCACCAGCGTCCGGAGCGGC
TGTGGGGTACGGGAACCTGCGCGCCGAGATGGCCGAAGGTCTGGTGGGTGTAGAGGGCATCCATGACGAAAAGGAGAACCATGCAAACTGAAATGACCGCTGT
CCTCTACTAGAGAAGANNGAGAGCTGCATGCGATATCGCAACTGCAGACAAATCCGGTACACTGTAGAGATGTACTCAGTCATITAATGGCGCATACTGTATA
TATITIAGTCTGGGCTAATFJTGA1TITITGT
>‘000127a-088.scf’ came from CONTIG 40 at offset 24;
“E:\SEQUENCE\export\EST_db\000127a\000127a-088scf” (50>618)
TCGCGAACAAGCATCATGAGCTTCAGCACCCAATCCACC7FCTCCAACTACCGGTCCCTGGGCTCCGTGCAGTCGTCGGGCCACCGGGTCCGACCGGTCAGCA
GCGCGGCCAGCGTCTATGCAGGCGCCGGGGGCTCGGGCTCCCGGATCTCCGTGTCCCGCACCACCAGCGTCCGGGGCGGCTGGGGGTCCGGGAACCTGGGCGC
CGGGATGGCCGGGGGTCTGGTGGGTGTAGGGGGCATCCAGGGCGAGAAGGAGACCATGCAAGACCTGAATGACCGCCTGGCCTCCTACCTGGAGAANGTNGAG
AGCCTGGAGGCGGATAACCGNAGACTGGAGAGCAAAATCCGGGAACACCTGGAGAAGAAGAACCCCAGTGAGAGACTGGGCGCAFI0ACTGTAGATCATCGAG
GACTGAGGCTCATATITrGCAAYrCTGGGACACGCCGCATCGTCTGCAGATGATATGNCCGTCTGCTGCTNTGACTCAGAGTCAGTATGAAAGACTGCCTGCG
CAGCTGGGAGAGGACTACCGGCTCGCAGTCATGTGACACATGTCACGCTGCGTGA
>‘000127a-045.scf’ came from CONTIG 41 at offset 0;
“E:\SEQUENCE\export\EST_db\000127a\000127a-045scf” (52>359)
GCACGAGGCAATCT7FFCGCCATCCTAGCCGAGAGGGTATCAAGTCGACATCTGCAGGAGTCAGACACGCTCGGGGCCTACTGAGAAGCCTCCCAGACGC117
CA7FITCTCTCTCTTGGGThI7ACGGTAGGGCACGAAGAGGGTGAGCTGAAAGGTFfGTAGAAGCTCCAGflGCTCGCCACCCTCCTGGACTGNAGAAACAGG
NCCCITCCAGGGATTCGTAGCGGACTAGTGGAGCCGCAGGNACTAAAGCGGCGGCGCGCGCTCCGNAATCCCNNATCTGGGTCCANAATACACANCTANATNN
GCTFJ
>‘000127a-046.scf’ came from CONTIG 42 at offset 0;
“E:\SEQUENCE\export\EST_db\000127a\000127a-046.scf” (48>562)
CGGCGGCGArJGTGGTGACTGAGCGGAGCCCAGCGACAGGATGGCTGGGCACAGATTGGTG1TGGTArFAGGAGACCTGCACATCCCACATCGGCGCAACAGr
FrGCCAGCTAAGTITCAAAAAGCTGCTGGTGCCAGGGAAGA1ITCAGCACATFrCTCTGCACTGGAAACCYfTGCACCAAAGAGAGTrATGACTATCTCAAGA
CTCTGGCTGGCGATGTCCATATFTGTGAGAGGAGACTTCGATGAGAATCTGAATFJATCCAGAGCAGAAAGNTGTGACTGTJTGGGCCAGTGANAATGCTCTG
ATCCATGGACATCAGTTATFI0CATGGNGAGATATGCCCAGCTAGCCCTArrGCAAGCAGTNGATGGGACA1TCTPATFFCAGACATACCATAATFrGTAGCA
TTGGCTGNAKFrATTCTC1TAIFrCCGTrCTGCCCTGAGCTATATGTCrFGGNNACACATATTCTCTFITGGTGAGTAITCAGCFfACGTGTC1TFGTGTFAT
ATGGAGAGAAA
>‘000127a-048.scf’ came from CONTIG 43 at offset 0;
“E:\SEQUENCE\export\EST_db\000127a\000127a-048scf” (54>584)
GCACGAGGGTTGATAAGGCATCTACGITErAGAAGCTCTGGCCACTAGTCG7FFAAGATGATGGCTCTGATGGCAThICTATGGAYPATAACGAGTCATCTGT
GAGAGAGAGTCACTCTGGACAGGC7FI0GTTACGCTGACTGACCCAGAGGTCCTGGGGGGAATGGCACCTTGTCCTCGCTCTTAAGAGAACCTGTGGAAGGAA
ACACAGAGTAAACGTGGCTGCCGThICACAACTGTGGAAGGAAATGTGTGAGCGAATGAAGGATC1TAGAAYICAAAGTAGAGGGAAGCCCACCnGTCTACTG
ATFITTFrGATGTATA2FrCACAGCGTCCTThFAAGATCTGNGAATGAGACTCTTCTAANCTCTATACTCITGCACTCTAACGCAGATCACAGTCTTATATAA
CTATTTATCNANNNAAGTATCAnCTAAGATGTrNTITNGGAAAACTGTAGAATGTAATGTACTATGAACTGATATCTGTCAAGTATTrATATAATACTGTTTA
NTTTACTGTITfTGGTGATCTA
>‘1000 127a-049.scf’ came from CONTIG 44 at offset 0;
“E:\SEQUENCE\export\EST_db\000127a\O00127a-049scf” (53>599)
GCACGAGGGCACAACACTGAGAAAACTAGGGAAGACTCAGGCTGAGAGCATGGCCTGTGTCCAGGATAGTCACAGTAnG1FCAAATAAAGAAGCFICCTGGCA
AGTCAGTcTAGCTAGTATCCTATGTATCGIrCGYI0GA7ITI17CAACACr2CTGAAGTCTCCCTCTAnAGTCTTCTCCCCAAGGGACATATGAGAAGACTCC
TGGAGAAAAATCTGITI7AAAATATGTGCTCTGTITGCTGTGAAUCCAGCTGCTGTGACUAATAAGGGCAGATGAAAGGCACYCAGAGATGGAGCATCmGGTA
ACATACCAAnGGGACACTATCAGCAGGTGGGGCTGACAGAAnnACCCATGGGTCThCATCACACCCCNCTATCCCATCCTCTNCATGGAANTACATATAACTA
AATCAAAGAGAGCnTCTcGAGCTATACAAT
>‘000127a-050.scf’ came from CONTIG 45 at offset 0;
“E:\SEQUENCE\export\EST_db\000127a\000127a-050scf” (52>430)
GCACGAGGCCCACCCCCAACCTGCGGGCACACGAGGAGGAGGCGAGGGTGGCCGGGCTGGGCTGGAAGGTGAGGCTGGACTCCTGGTAAGTGTCAGGCTCCCT
GGCACAGGGCnCCCTGTGUGTATAATCCTGCTTrATAAATAThTAACTCCCACTGTTAGGAAnGCTnGAGGCAGAGACTGAGflCCTGGCGGGCTGCTGCATG
GCCCCTCFAGTGGCmGNAGTGGACAGGNTGThGNAGmGIGGGCAGGTGTAT17CTGAAAGCCCTFFPGTCCATAGGCT
>‘000127a-051.scf’ came from CONTIG 46 at offset 0;
“E:\SEQUENCE\export\EST_db\000127a\000127a-051scf” (43>355)
AAGCAAAThrTCTGCAGAATCACAAATCCATGAGCTGAGTGACTGTGGCGGATCAGTCTCTAATCACCCCTGTGCTAGAATGCTCYI7ATGNNGTAGNTC
>‘000127a-052.scf’ came from CONTIG 47 at offset 0;
“E:\SEQUENCE\export\EST_db\000127a\000127a-052scf” (48>395)
ClTAGTGGATCYGTGTGGCGTGCCTCTCTGCTCTATGCGTGCTGIGGCCGTCACACCTATGCCGAGCTAAArFCTGCCAAYrATCACAAAGCCATGAACTGTG
TGACTGCGCGGTGGCAIAGCGGTCCTCTGGGTGCTGCTGTCCCYfCACThfCCGTCCCCTCTGTGCGTACTATCGAGAGATGAGGCTGAAATCTATGAAGTAC
TGGGTGAAAGAAAGGTGAGAGGGGCTGJGTGACCATGATCTCGCTTACAGAAGAATAAGGCACCCCCGTGCTAGATAAGCATGATTGAGGCCCGCATICCAAG
AAGACT
>‘000127a-053.scf’ came from CONTIG 48 at offset 0;
“E:\SEQUENCE\export\EST_db\000127a\000127a-053scf” (49>527)
CAGAGGTGCAAC1TTC7LTCGGTCGTCCCGAATCCGGG1TCATCCGACACCAGCCGCCTCCACCATGCCGCCTAAGYFCGACCCCAACGAGATAAAAGTCGTG
TACCTGAGGTGCACCGGTGGGG1GTCGGTGCCACGTCTGCCCTGGCCCCCAAGATCGGCCCTCTGGGTCTGTCTCCAAAAAAGGTCGGTGATGACATCGCCAA
GGCAACTGGTGAnGGAAGGGTCTGAGGAHACAGTGAAACTGACCATTCAGCAGACAAGCCCAAGAGGNGGTACCnCTGCnCTGCCCTGATCATCANAGCCCTC
IGGCCCACCAGGNACAGANAGAAGCAGAANAACA7FITAAGCACAGGGAANACKFIACHnGAThNGAGATCGTCACATflGCCGGNCAGATGCGCArCGGTCT
CTAGCTAGAGATCT2CTGGANCAAGAGACTGGACGNCCACCTGTGGN
>‘000127a-055.scf’ came from CONTIG 49 at offset 0;
“E:\SEQUENCE\export\EST_db\000127a\000127a-055scf” (48>610)
TFUCCCAGGTGCTGCTGGCCGAGTCGGTCCCCCCGGCCCCTCTGGAAATGCTGGACCCCCTGGCCCTCCTGGCCCTGCTGGCAAAGAAAGCAGCAAAGGCCCC
CGCGGTGAGACTGGCCCCGCTGAGCGTCCCGGTGAAGTCGGTCCCCCTGGTCCCCCTGACCCCGCTGGTGAGAGGAGCCTCTGGTGCTGACGGACCTGCTGGA
GCCTGGCACTCCTGGACCTCAAGTAnGCTGGACACGTGATGTGCGTCCTGCTGGTCAGPGAGAAAGAGCATCCCTGTC7FFCTGCACCTCTGTGAACCAGCAA
AC1GTCATCTGAGCAATAGAGAACTGCCCCCTGTCCCATGGCCCCTGA7FFGTTGTACCCTGTGAGFI7GTACGGGAGGAGTCTGGGTGAGTACCrGACAAAT
GTCGTGCCAGGGGAGGGGGGAACGCCGACCGGGCGGCTCCGCGGCCCGCCTGCGACTGCGCAGAGGATGGGGGACGGCTGGGCCTGGTCGCCGGGGCGGGCCG
GGGCCAGCCCCGGGAGGGGAAGAAAAGGAA
>‘000127a-056.scf’ came from CONTIG 50 at offset 0;
“E:\SEQUENCE\export\EST_db\000127a\000127a-056scf” (49>595)
CTCGAGTITHTIY1YITITITFFAThFrCCI1YrAArITITCTGAAGGATATACACCACATATCCCATGGGCAATAAAGCGCAYJ7CAATGTGTh2ATAAGCC
AAACAGTCAC7ITJGThI7AAGCAAACACATGTACAAAGTAAAATAAAACCACAAAATAATGAACTGCATG1TCATAACATACAAAAAYJGCTGCCTACTCAG
TAGGTAACTACAACAITCCAACTCCTGAAYI7ATAThrATAAAThI7ACATINTCAGThI7AAAAAAATAGACTFr)CCnGTITLTGCTCCCAAAGCCTCCCC
CCGATCACCACTCCCYrGCCCCCTTNAGCTAGAGGTGAGCACATCCCTCACAATTGCACTGTCAGNCCGNGTCAGCAGGNCGCATCACACAAAGGCACCCAGA
GTGNAANC1TNTTAANCAAAAGNNACAAAAAACTACI7CAAAAAAGAGAAAAACAACGNNATGCNCTGGGA
>‘000127a-057.scf’ came from CONTIG 51 at offset 0;
“E:\SEQUENCE\export\EST_db\000127a\000127a-057.scf” (49>619)
GTGATAYrCAAACGAATAGTCCGTCAACCCCAGACACTGGTI17GAAGAAATTGAGACTTGATCATAGGACTGTATAGTGCACAGCGCCAGCATGTATGCTAG
GAGCAGTGGGAGGAGGCCAGTAGAAAGCC7FGTCATCThIAGGGGTAGTGATGTGACTGCTAThFGGAGTGTCACTGAAAAGGAAAACITJAGCATGCTCACT
GATCTGCCTATAGCTCCAGCAACAGCTCGGATGTGCGTTCTCCAGCCATCATGAGGCTGAGTCAAGTTCGTCTCTAAGTCAGAACAGCAGKFITCAGCTATGA
CATTCTGATTCAAGACATI1TGTFFCAGGAATCAGAATrCTGTCTArrAGACTGGGACAGCTGNGGCAAGCTAAATTGCCTGTNACAAGCCAGAYnTI-IYIT
AYPGATACTGTAATAYfGTGTGTAIFI7ATATATA1TGTACGNTATCTAAGITM1AAAAGTGTTGTGCTTTYPGNTFITGThYFPATGCYPGATATFI7CAGA
GYYAGCTCATmTGACACATAGTAGACGAAGCTGrIGATATCAAGCAGATGAATCAATAATTTGG
>‘000127a-059.scf’ came from CONTIG 52 at offset 0;
“E:\SEQUENCE\export\EST_db\000127a\000127a-059scf” (50>595)
GCACGAGGCCGGTGTCCCCGCGCCAGAGACGCAGCAGCGCTCCCTCTGCCCACACCCACCGCGCCCTCGCGCTCGCCTCTCCHCCGGAGCCAGTCCGTGCTAC
CGCAGTCGCCCAGTCCACCACCACCCTCTGCAGCCATGTCCACCAGGTCCGTGTCCTCGTCCTCCTACCGCAGGATG7FJCGGCGGCCCCGGCACCGCAAGTC
GGCCGAGCTCCACCCGGAGCTACGTGACCACATCCACCCGCACCTACAGCCTGGGCAGCGCGCTGCGCCCCACCACCAGCCGCACCCTCTACACCTCGTCCCC
GGTGGNCGTGTACGCCACGCGCNTCTCGGNCGTGCGCCTGCGAGCGGNCGTGCCCGGCGTGCGGNTGCTGCAGACTCGGTGGAC1TCTCGGGCCGACGCCATC
ACACCCGAGTCAAGACACCGCACAAGAGAAGTGGAGCGCAGGACTCATGACGCTCGNCACTACTGACAGTGCGCITCGGACACAAACAGTCTGTGCTGAGTGG
CACTCAGGCAAGCAGGCGCGGGGAC7ITAA
>‘000127a-060.scf’ came from CONTIG 53 at offset 0;
“EASEQUENCE\export\EST_db\000127a\000127a-060scf” (1>277)
AGGGCGGYJ7AATAGTCCGCGAGACCGTCCCTfCTCAACCCAGFYGAAGAGCTC7FfATC1TCAAAAAGAACTCYfACTCAAGLTAAACATCACAGGGCTGAC
TACACTAGGGGGYfYfATTGCCTCTGTGCYPGTTCYAAATCTG1T1TGGACGATCGCTACGAATCACTATGTCAATCAGCAAGGTGAAGAACTAAGACATGAA
GGAGAACCGGATGTCCYrGrrGTrGGCCTCTCAITrYFFFGACYrGGGGAAGACGAGGrErGGCTGGiN
>‘000127a-061.scf’ came from CONTIG 54 at offset 0;
“E:\SEQUENCE\export\EST_db\000127a\000127a-061scf” (55>678)
GCACGAGGCTGTCACTTrGTAAAGCTTTCAATCAAAACCACCCTGAAAGCACAACAGCAGAATrCAATGATTCTGACTCTGGCAiTTCACTGAACACAACAAG
CTCCAAGCATGGCATCACCAGACCACTCAGTGGAATC7FJCCATCTATGGAGACACA7FJ7GC7FI7GGCYFCAGTGATTCTGAAATGGAAGAGATAGATAGT
ACCCCTGGAAATGTCAAACAGAAGGGGCCCAAAACACCGCCAGTGTGGCCTCCTGGGGACCCAGTCCAACC7FJTGTCGTCATCACAGGGGAACAGCGCTGCA
GCACGCGA1TCCCAGAGTGAAAACGCACCAAAGAAAGAGTACCTGTAAGTCCGGGTCATCGAAAAACGCCAYrCACATAAGACAAACAYfCAGCCGC1NGGAG
GCTCACCTCACAAGAAGAGAGCTACGGNNCAAAGCTCTCATATCCATTCCTGTAGAAAGACAACCTCCAGII1GAGAC1TCATGAATGAGTGCAGGAGCArFC
ACGAGCTCACTGCATAAiTTAAACTACTAGAGGGNAGATAAGGGCTGTCAAAATGCAAAAGAAACGGAATATAGGNACGGGCAGATTAATAITAAAAAAAAAG
AAAAAGTCAAG
>‘000127a-062.scf’ came from CONTIG 55 at offset 0;
“EASEQUENCE\export\EST_db\000127a\000127a-062scf” (47>628)
CTGACAGTGAGCCCGAGTCCGAGGTATITGAAATCACGGAC7ETCACCACTGCCTCGGAATGGGAAAGGrFrATTrCCAAAGTFIGAAGAAG7Fn7TGAATGA
nGGAAKITGATrGGAAACTCTGTGGGAAAGCCACYFGAAAAGGGTATA7FfTACTTCTGGGACATGGGAAGAGAAATCAGATGAGATCTCC1TI7GCAGACTT
CAAG2FICTCAGTCACTCATCAYITATC7FPGTACAAGAATCCACTGATAAAGAAGCAAAGGATGAAGTACTAGAAGATGYrATFrCCACAACCTATGCAAGA
ATTGCTGTGTATGAATAATGACrITCCTCCCAGAGCACATFrGCCTGGNAAGATGGNATGGNACTCGAGAGNTNGGGNNGATAGCACCCTGCTGCAACATGAT
GCTGTCCTCATGAATCTAAGTGCATCTTCTCTGACTCTGTGTCTATGGCITGGAAACACTGCTGNCAGNGCCACTCrrGTGCAGATCTCACAATGGCGAGATG
ATGGGNNGAAGTCAGGNNCTGGGNNCGACTGATL1TGAATGGTCTCTCGAAAGGCCAATAGATACTATJTICAGNCTGT
>‘t000127a-063.scf’ came from CONTIG 56 at offset 0;
“EASEQUENCE\export\EST_db\000127a\000127a-063scf” (48>639)
GAAGGA7FITPCCAG1TAGTfTCTCCATGT1TGCCCGAAGCCCAAAAAGTCTCTGTCCAGTFCTTITGTfAGGACAGTAAGACCCAGTGAAGCCCAAGYI7AA
GTGGAAGGCCCTGAATAAIITCCAGAAGACCCAITCYFATCCYPAAAATCCTAGAGTGGTCAAGGGACAGAGTGATTTGTFJTCCGAAAGAGTCCGAGGTATA
TGTG7FPATACAGTGTCAGThfGGAAAGCTGATGAAG7FLTCGCAYICThITrAGGAGGTCAAGAAAGGCACANNAATGCTGCTGCAGAAGGNGGNNGGNNGT
CA1TN1TGCCAGrJGTCTAGCTrGA1TGCAGACGGITCTGTNAGTGTTATGGNCGTGTATGTATCACTCAJTCATGGT117GATGAGTAAATATGATGAAATG
NCTGANACTGAGGCTGGNATATACAAATGCAGCTGACCTATCTCAAGACTACGTCAGCAGGTAGCAACTAACATATCTAGAGTATATTTGTAATITGATATGA
ACAGAA7FACGAGTGGG
>‘000127a-064.scf’ came from CONTIG 57 at offset 0;
“E:\SEQUENCE\export\EST_db\000127a\000127a-064scf” (50>649)
I17CCAGTCCTGThI7CCACCTACAGAGTCCCAACGTGGTI0ATI0GCAGGCAACGTTCCCTCTCTCCGTGGTCTAGAAGCCCCTCCCCCAAGGTAGAAAGAA
GGGAAGAAGCTAACTCCAGTGT1TCCGTITGCACTGATCCCCAAYJ7CAGTCCAGGAGGGGGCYGGTAACCCCTGTCCGTCAATATCCTGGCACC7FI7GGGC
7FI7GTGAACGCCTCCTAGCCAAATCACTAGAGTACAGTGACCCCAGCCTCCTGCCTGTCCCGAGTGAGCCCTCCCCACCCTGACCGTGCTAACTGTGTGTAC
ATATATA1ITCTACATATATGTATATAAACCCGCACTGCCATGTGTACCCTEFCTGTGGTGTCTAGCATFPAACTFI7AI7GTCTAGGCCGGGCGGGGTGGNA
GGNAAATGCCACAGTGAGGGNGTGGCAGAGTCAATTGCTATATATCGAAAAGAAAACYPYI17AACLTNATLTCACATGCATCTCAGAGATATTANAAGYJAG
GAGGGGGAGYJ1GGAGTGGGAAAAACIFJAGGGAGGAGCTGCTGTGNAGAAGAACAATGCTGGAGACCFJ7CACCAGCACCAGCGCC7FI7GCCTGTGGCA
>‘000127a-066.scf’ came from CONTIG 58 at offset 0;
“E:\SEQUENCE\export\EST_db\000127a\000127a-066scf” (53>292)
GCACGAGGCCGGTCTCCGCGCGGGTCTGGGTCGCGGAACCCGGTGGCTGCTGTGCGGGCGTCATGTCAGACAACGAGGACAAThFJGATGGAGACGAC1YrGA
TGACGGGAGGAGGATGAAGGGCTCGATGACTrTGGAAATGCCGAGGAGGAGGGCCAGGATAACG7FfGAGA1ITCTCCCCTFfGGAGAGCGACCGCGAGCCAA
CCANAAACCAATCACACACCATATATGACCACTATGA
>‘000127a-068.scf’ came from CONTIG 59 at offset 0;
“E:\SEQUENCE\export\EST_db\000127a\000127a-068.scf” (49>600)
GCTCTFrCTFfTGrnCCCAGCTACAACTTGCAGAGCCATCACATCCCAGGAACATTTAGGCAAAATATAAAGGTGTGTGTCTAAAATATGTCTFITGAAGAAG
CACTTCCTCCTTCCTCAGTGC7FITCTGTCCCAC7FI7GGCTTCTGCT1jGGCCACCTGGGGCTGCCGCAAAGCAGGAGTCCITCTGGCCCTGGCTGTGAGTG
T1TTGTGTCCAAAAAGTGrrTAAGGCGCAGTGGAAG7IITAAAGAAGTCTAAGACACAGTCCYrGrrCrrAAkAAGATCACCGGCAGGNTAAAANTACAGTGC
TAAAATATAAGCGTATACCTGAGAAATCKFrCATGTFI7CAGCTGYI7KITGGAGTGTCTGCTAGGTGCAGACTGTAGGGTGAATATAATAATAAGAATAGAC
TGNTGCTCCTCAGCAGTTCTGTCTCTCAGAGGTATCGAGTATATATCAGATAGGAATA7FrGGAGCCCTGCATGCCTTGCCATNAAGCTCAGGAGGCAGATCA
CGATGAAGGACGGCGAGTAGCGAGGATGCCmTGNATAACGN
>‘000127a-070.scf’ came from GONTIG 60 at offset 0;
“E:\SEQUENCE\exportkEST_db\000127a\000127a-070scf” (48>619)
CGGCTGTGTCTTCTCTGCTCTCCCAGCCGCCGCTGCCGACCCCGCCGCCCGCCGCCCGCCGGACTCGGACGCGTGGCCGGCTGAGCCTGACCTGTGGCCTGTC
CCTGGCCCTCTAGTGGAGCCTCCAGGCGGGCHCTGTCTACGTGCCCTACAGAGGAGCCATCCCACCAGCCAGACGCTCCCAGTCGTCACCCAGGCAGGCGGTP
GGCTTGGCCCCAACCTGGGCTGGACACGTGTCCCCCGCTCCTCTCCCCGCTGGCGrGGCTGTCCTCCTGGGCAGCCCGTCCCCCACCCACTGGNCCCCAGATG
GGCGAGGGAGAGGTCGACAACATGTGGCACCGTCTGCCTCAAGTACCTGCTCF1CAGCTTCTACTGCTGTCTGGCTGGCTGflCTGGCCGTCATGGCCGTGGG
CATCTGGACGCTGGCCCTCAGAGCGACTACATCACCTGCTGGCCTCGGCACCTATCTGCCACACGTACTNCTGGGGNGGGGGCATGYFGTNTGGGACCGGGCC
TGGCTGTTGGCACCTCAGGACGAGGACTGCTGGCGTCTTP
>‘000127a-071.scf’ came from CONTIG 61 at offset 0;
“E:\SEQUENCE\export\EST_db\000127a\000127a-071scf” (52>598)
GCACGAGGAGAAC1TTCAGGGACCCTGTGAAGCAGCTCCAGCCGAGATGTGCGAGGAGGAGGACAGCACCGCCTTGGTGTGTGACAATGGCTCTGGGCTCTGT
AAGGCCGGCT1GCGGGGGATGATGCTCCCAGGGCTGErCCCATCTATCGTGGGACGTCCTCGACATCAGGGAGTGATGGTGGGTGGGACAGGACAGCTACGTG
GGTGATGAAGCACANAGCANAAGAGGAATCCTGACCCTGGTACCCGATAGAGCACGGCATCATCACCAACTGNGACGACATGGAAAAGATCTGGCACCACTCT
CTACATGAGCTTCGTGTTGCCCCTGGAGCATCCAACCCnCTCACCGAGGCGCCCCTGNNACCCCAGCCANCGGNAGNATGACCAGAATGNnGAGACnTCATGT
CCAGNCTGTATGTGGCTACAGCGGCTGTCCCTCTACGCCTCTGGCGCACACTGNCATGNGCTGACTTGAGAAGNGNCACCACACGGCCATCATGAGGCTCGCC
TGCCC
>‘000127a-072.scf’ came from CONTIG 62 at offset 0;
“E:\SEQUENCE\export\EST_db\000127a\000127a-072scf” (49>521)
TCrrAAACCTGAAGTGGACTACACCATCACTGTCTATGCTGCCACCGGCCGGGGGACAGCCCGGCGCAGCAAGCCCGrL17CCATCAAYrACCGAACAGAAAT
TGACAAACCATCCCAGATGCJGTGACTGATGCACTGCTCCTAAAAATGGCCCAGGACCATCGATAACGACCTGTGAGTCCAGATCCAGATGACAAYFGAAGGC
YGCAGCCCACAGTGCAGTATGTGGACAGAGTCTATGCTCTCCGGAGAGAGTCACGCTCTGGCAGACAGCGGNTACCACCAACTGCTGCGATCCTACCTGAGAG
AGTGACACCCTACCAGCTGACTGCCAGCGACGGCACGCATGmAGCTCATGGGTAGGAGCCGGGGA
>‘000127a-073.scf’ came from CONTIG 63 at offset 0;
“E:\SEQUENCE\export\EST_db\000127a\000127a-073scf” (49>374)
CGGACAYFCAGACAGAACGTGCGTACCAAAAGCAACCGACCATCmCTAGAGGGTCCTGCYFGGAGAAACTGGCAAAGAAAAGGTCCCTCGATACTACAAGAAC
AGGTCTGGGCCGACTCCAAAGGAGGCCATCGAGGGCACCTACA117GACAAGAAATGCCCrmACGGGTAATGTCTCCAnCGAGGGCGGATCCTGTCTGGCGTG
GTGACCAAAATGAAGATGCAGAGGACCATCGTCATCCGCCGAGACTACC711TCACTACATCCGAAAGTACAANCCGC71TGAGAAGCGCCACAGACAGGGCC
>‘000127a-074.scf’ came from CONTIG 64 at offset 0;
“E:\SEQUENCE\export\EST_db\000127a\000127a-074scf” (49>601)
AAAAAGAACCAYFCGGATATATGGGAATAGmGGGCTATAATGTCAATCGGAUTCTAGGTCATCGTATGAGCCCACCATATATI7CACTGTCGGAATAGACGTC
GACACACGAGCCTACCACATCAGCCACTATAATATTGCTATrCCAACCGGGGTAAAAGTCCAGCTGAGGCICACTTCATGGAGGTTATCAAATGGTCTCCTGC
TATAATGTGAGCCCTAGGCmAThFITCUAmACAGTAGGGGGAACTGGAATTGTCYrAACCAACTCTTCCCTCGATKFI7GGTCYI7CACGACACATACTACGN
TGNCGCACAmCCACTATGTTL1TATCAATAGGAGCTGNA7FPTGCTATATAGGGGAThIGNTCA1TGATTCCACTATCTCAGGTATACTCTCACGATACAGAG
CCAAAATCACTCGCATATKFI7GTAGCGNCATATAACCTCTCCACACACCTAGACA2FPGGCTGCTCGCGAACTCGACACAGAGCTACCATAGAAACTATCAT
AGCTATCTTCCTACGAG
>‘000127a-075.scf’ came from CONTIG 65 at offset 0;
“E:\SEQUBNCE\export\EST_db\000127a\000127a-075scf” (55>670)
GCACGAGGGGTATCTGCTCACGCCATGAACAACTCTGGCCCCACCGCCCAGATCAITGAGCGGGAGGGCTGGAAGACCAACATGGACrGnGGGCATCGGAAAG
CTGTGACTGTCGTGAArCAACCCTMTC1TCAAGAAGAAGCAGAAGAATGGCAGCTCCGCGAAGCCCAGCTGCCCATACTGCTGCTGCGCCGTCGGCAGCAAGG
ACCGCTCGCTGTCCGTCTGGCTCACGTGCCTGAAACGGCCTIYI7GGTGGTCATCCACGAGCTGTfTGACAAATCCATCATGGACATCTCCTGGACCCTGAAT
GGGCTGNGCATCCTGGTATGCTCCATGGACGGCTCCGTGGCCThFCTGGACTrCTCCTAGACGAGCTGGGAGAACCCCTGAGCGAGGANAGTAGAGCCGCATC
CACCATCCACTACGGCAGAGCTGGCCTCATGACGAGCCCACTGTCACGCTGTCATGAGACCAGAAGCTGAGTACAGGCAGCGCACAGTGGACGCAGGNCCCAG
NCAGGACGCGACCGGNCAGAAAAACC
>‘000127a-076.scf’ came from CONTIG 66 at offset 0;
“E:\SEQUENCE\export\EST_db\000127a\000127a-076scf” (50>5 18)
TGGAGTJTCTCCCACCCACGCCGG11TCTGGCCTGCCKITGACGACGGGCTGCGGCGGGCTGCCTATGAGCGGGGCGTCAAGGTACGCCTGCTGATCAGCTGC
TGGGGACACTCTGACCCCTCAATGCGGGCCIFfCCTGCTGTCCCTGGCTGCCCTGCGTGACAACCACACCCACTCCGACATCCAGGTGAAACTCITJGTGGTC
CCTGCGGACGATGCCCAGGCCCGAATCCC10TATGCCCGCGTCAACCATAACAAGTACATGGTGACTGAACGGGCCACCTACATCGGAACCTCCAACTGGTCT
GGCAGCTACYITCACCGAGACGGCAGGCACCTCGCTGCTGGTGACACAGAACGGNCGNNGTGGCCTGCGAAGCCAGCTGAGGNCCGTGTITCCGGGNTCTCCC
ATCCCTGTCCCTGTGCCCNCCGCTCTGTFJGACCCGNTGTGATCANCAGGCTCCTNTCGCAACC
>‘000127a-077.scf’ came from CONTIG 67 at offset 0;
“E:\SEQUENCE\export\EST_db\000127a\000127a-077scf” (55>522)
GCACGAGGCAGTGTC7FJTCGCAGGTJCGTGGTGCGGACCATGTGTGCAGTGCTGGGGCTCGTGGCCCGGCAGGAGGACTCTGGACTCCGGGATCACCGTGTC
AGGGTCCTCATFI1TCCAACCACGTGACACCmCGACCACAACATAGTCAACflGCTCACCAGCTGTAGCACCCCTCTACTCAATAGTCCCCCAAGCHGTGTGC
TGGTCTCGGNGCmATGGAGATGGATGGTCAGGGCGAGNTGGTGGAGTCACTCAAGAGA1CTGTGCflCAACAAGGCTFFCCCCCTACCCCTCTGCTGCTATFI
7CCCCGAGGAAGAAGCCACCAATGGCCGGGAGGNNGCTCTGCGCTFI7CANYrCCTGGCCATITFFCTAUCATGATGTGGTACAGCCTCTTTACTGNCGAGTN
CAGAGACCTCI17GTCTCGTGACGGTGTCAGATGCATCTGGGTCTANAATGCTGGTNT
>‘000127a-078.scf’ came from CONTIG 68 at offset 0;
“E:\SEQUENCE\export\EST_db\000127a\000127a-078scf” (1>349)
CCCGGGGCGGCGrrAACTAGGTCCCGGCTGAGCGGGCGGGCATCCCAGAGGCCAAmGCTGACCCTGGCCCAGAAGGCAGTGGAGCTGGGCTCGCTGCAGAACA
CAAAGGATGCCAGTGGCTCTGAGGAGAAGAGAAAAAGTGTGYrGGC1TCAACTACCAAGTGTGGGGTGGGAGThPTCTGAGCCTGCC1TANGCAAGCGAGCAC
GAGAGGACAGCGGGATGGTACCCCTCATCATCCCAATGTCTGTGCCTGTGCGGGCAGTGGACCCACTGANGCAGCTCAGCTGNAGGTGTGATGAGATGAAAAG
GTNCCGACAGCACCTGCTGACACAACCGTCATCAThI7G
>‘000127a-080.scf’ came from CONTIG 69 at offset 0;
“E:\SEQUENCE\export\EST_db\000127a\000127a-080scf” (22>530)
TTACCATGGATCCTCCGGACTGCATGCAAGCTGAGTGTCJTCCC1TGCCGTCCCCGCTCGTACAGTCTCGCTCATCTCGTTGCCGCCCAGTCCCCGCGCCCCC
CGGCCGTACGAGCATCCGGCCCCCGTAGCGGACGCCATGCTGCGGGCACGCCCCGTGCThFGGGCTGTGGThTI7GACCGCACTGACGTTGTTCCGCGGTCCG
CCGGTGGTFrCGAGCTGGGGCGGGCACGATGGGCGCCGGCCCAGTGGTGCGCTGCGAGCCGTGCGATGCGCGTGCCGTTGCCCAGTGCGCGCTGCCGCCCCCC
TCCTCCCGTGCGCCGAGCTGGGCGCGAGCCGGCTGGGATGCTGTCTACGAGCGCGCTGCGCGAGGTCAGCCTGCGACGCCATCCGAGGGTGTGTh7CCGGCTC
G7FrGTCGTCGCGCTGTGATCGCGCCCG7FI7CAGGTTGTGGTGGCGGGGCTCGGCCATGCGCGCGTGTGCGCTGCC1TACCGTrCGTCGGTAGGATGCGTGG
>‘000127a-081.scf’ came from CONTIG 70 at offset 0;
“E:\SEQUENCE\export\EST_db\000127a\000127a-081.scf” (47>636)
AAYrGGCCCGAGGCGGATCGCCCCTCGACTGCAGTCmGCATCCGAGAGACCATGGTGGGCTCCCCGCGCGCCCCACTGCTCCTGCTGGCATCCCTGATCGTCG
CCCTGGCCCTGGCCCTGGCCGTGAGCCCCGCGGCAGCGCAGGGCCCTAGGAAGGGTCGCCTGCTGNGCGGCCTGATGGAGGCGGACGTCTGAGGAGGGCGTGC
AGGAGGCGCTGTCCmGCGGTCAGCGAGTTCAACAAGCGGAGCAACGACGCACCAGAGCCGCGTGGTGCGCGTGGTGCGCGCCCGCAAGCAGGTCGTGTCAAGG
ATG1CTAmCGGACGGGNAGCTFI7GGCCGGACTACATGTACCAAGNNCCCAGCCATCTATACAGCTGTCCrICATACCAGCCGCACCTGAGAGGGAAAGCTGT
GCTC1TNCAGThTACGNCGNCCATGGATGACACATCANCTGNTGAANTNAGCTGCAGNATCAGCAGCCACTGACCGCCATCTGCTCTGCGGCCACACTGGNGG
GATGCTATGGCGGCCTCCCATGCGCCTGCAACAGCTCTGGCATGNTGAGC
>‘000127a-084.scf’ came from CONTIG 71 at offset 0;
“E:\SEQUENCE\export\EST_db\000127a\000127a-084scf” (49>‘316)
rAGGAGCCAAGGCAATrCAGCTGAACAGTAACAGTGTTCAAGC1T“GCTACAAGGGAGCAGCGCUAAAAACATGGGCAGAGTCCAGGAAGCAATAATACACTT
TCGGGAGGCTATACGTCGCGCCflGTATGGGCTATCATTGTTACTAAACTCnAGAGCAAATGCACAAACCIIThACCmACCACCGC
>‘000127a-085.scf’ came from CONTIG 72 at offset 0;
“E:\SEQUENCE\export\EST_db\000127a\000127a-085scf” (49>‘427)
TGAGAGTGAAACTGCAGCCGGCGTCCAGCTCTAAGCTrCCTGC7FPTCAGTCCmGACGCCTCCAGCTGTGATCTCTCAGATGCTGCTGCTGGACAATCGACAC
AGAGCCCATCCGGACGGTATIGCTGACACAACCAGGGTGGACAGCCmCAGCGAAGTAGGAGAAGTGAGACCCGGACCTGACAGTCGGGTGCAGCCTGACTCCT
CCCATGACAGAGC2FrGCCGnCACGCnATGCTAATGCCrmGCTGTCTAGATAGGACTGATCATGGTGATAGTGCAGAGTGCCGAGCTGTCCTGACATCNAGCT
CTGGATGGCAGCCTCCGAC7FJA7FITGGANAGTGTGIFfGTGGT
>‘000127a-086.scf’ came from CONTIG 73 at offset 0;
“E:\SEQUENCE\export\EST_db\000127a\000127a-086scf” (49>‘664)
ThnFFFFrAAAAAAAGAnACAGAAAACACThFACTGAAFCCThGCTAAGACAGTCGAAGGATCTGAGAGACAGCAAGCACAACACAGTACAAAAGGAGAAGGG
AATGGCCAGTGCGACACTAACACAGCACAAAGGGAACCAGGCGGAAGCAACCACACAGTGGIflAGGCATTTATACTTAATCAGGAEVFEFAAGCYEPAAAAG
TCCAGCATAAAGGGGGNGGAGTGGATGGNGACAGGGGCTAAGCrfATCTACAATCACCAACCICACACTGGCTCICCACGTGAGAAGNGGAGGNTAAACCTGC
CTACAGAGGCCCAGCTAGAGCATGCCTAGGCAGTCACATITAGGTGTCGATGTCACA7FFGGCTGTACATGmAAGGGACGATTCACCAGACTGGCTCCATCAC
CTGGCTACGAAGP7GAGTFPCT17GCA2F17GCTCAGAGNCAAGCACTGNAGAGTCATCAATAGCTAGTGCTGYITACGNCTGGGNCAAGGCTCTATAACTCA
C117CTAGGAGmAGATACAGAAATCAGCACCTAGAGGAAA
>‘000127a-087.scf’ came from CONTIG 74 at offset 0;
“E:\SEQUENCE\export\EST_db\000127a\000127a-087scf” (50>‘630)
CTACAACCACCTCTACCTCCAAGGCAATAGGATCAATGAGCTCCATCAGCAGCTTCTGCACCGTGGTGGATGTCATGAACITCTCCAAGCTGCAGGTGCTGCG
CCTGGATGGCAACGAGATCGCGCAGCGCCATGCCCGCTGACGCGCCCCTCTGCCTGCGCCTGGCTAGCCTCATCGAGATCTGAGCGCCACTGGGCGCAACAGA
CCGCGTGACAGAAGNCCATGGGCTCTCTCTCTAGTCnCYICCCTGTAGGCAGNThTAGGGGNAGNCAGGGAGACAGCAGCNCTGCTGAAGGACATGACACGTC
CGCCGACAGIGTGGflGGCAGAAGGTNGTAACCCTGAAGNCCAGNCCCCGAAACTCATACCCTCAGTCTCACAGGATCAGGGNCTGACATGNCTGAGCATAAT
ACTGGCmGAGTATGCTGATFI7GGCAGACTGACGCTCCCGGGCGGCTGGCGGCGAACYrGGCCCNAGTGTGYITAAFrACCnGC
>‘000127a-089.scf’ came from CONTIG 75 at offset 0;
“E:\SEQUENCE\export\EST_db\000127a\000127a-089scf” (55>‘387)
GCACGAGGCCCTGTACACATATCCTGAAAACTGGAGGGCCCGGCCCTCAGCCGGTCAGTACAGCGGGGCTCAGGTCCGCGTGCTCTCCGCACCACCCCACCCA
nGGCCACCCCGCACCCCCGAAmCTCCGTAAAYffCCTGCTGGCAAGGnCCAGCCmGAGGGTGACGATGGACTGTGTGnCGAGAGCTGCCAGCCTACTATGTGA
GCAACGAGGAGGCGGNGGTACTCCCGAGGCAGCAGCACAGGTGGTGCAGGGGGTGAGCTNTGCTGATAGCGACATAGTGCCACCGCCAGCTGGGGG
>‘000127a-090.scf’ came from CONTIG 76 at offset 0;
“E:\SEQUENCE\export\EST_db\000127a\000127a-090scf” (52>‘432)
GCACGAGGCACCATCGAGAACGTGAAGGCCAAGATCCAGGATAAGGAAGGCAflCCCCCTGACCAGCGAGTCGACCCTGCACCTGGTCCTCCGTCTGAGGGGT
GGTATGCAGAYmCGTGGGACCCTGACCGAAGAGGGCANTCCCCCCGCCANCANAGCTCATCTIT2GCGGCAGCACTGGAAANAGACGACTCYfflGATACACA
TCAAAANAATGACCTGCCCTGTCCTCGTGGGGGGGGT
>‘000127a-091.scf’ came from CONTIG 77 at offset 0;
“E:\SEQUENCE\export\EST_db\000127a\000127a-091scf” (52>‘619)
TCCAGmGGTACCAATGATAAGGACTCAGACrrGTGTCTGGTTGTGAGAGAGAGTIGCAGAGAAGGAGTFFAACAGCATCAGTFJACTGAGGCCKF2CCCATGG
CCCGAGACTGGGAGCTGCTCCCCAGTGCTTCTGCCTCAGCTGAGCCACAATCCAAGAACCTGGCYICTGGGCACTGTGGCCCCGAGACCAGGTCCTCAGGCCA
GCGCITGTACCCTGAGATCYI7CTATGGCAGCCCTGGGCCTCCCAGnCTCACGTCTCAGGAGGAGCCATAGACTCTCAA7FrACATCCCAACAGTGGAGGCUC
CGTCCTGAGACACCCTCACTGCACTCDACAGATCACAGCCCCTGTACCTCCCCACGGTCCCAGCCCCGCCCTCGGCACTGCTCTCAGGGNTAGCTGTACAGGG
GCCA7ITICTGGATITCTNCGCACTGCAGCACAGTACTGNGTAGCTGNCGCTGNAGGGflCTCTACCCTCATCCnCTNTACTCAGCTCTGACTATCCTTGGCC
CCACCTGTTCAGTTGCGGGNTGCTGCTAGGTAGAGGGGG
>‘t000127a-092.scf’ came from CONTIG 78 at offset 0;
“E:\SEQUENCE\export\EST_db\000127a\000127a-092scf” (55>563)
GCACGAGGAGATGATCCAGCTGGGGGACGGTGCAGTACCTGCCCATCCTGCAGACCAGCTGAGCAACCGTGTCAAGGACCTCATGGTCATCAACCGCTCCAGC
ACTGAGCTGCCGCTCACCGTGTCCTACGACAAGATCTCGCTGGGGCGGCTGCGCYrCTGGATCCACATGCAGGACGCCGTGTACTCACTGCAGCAGnCGGAYF
CTCAGAAAGATGCTGACGAAGTAAAGGGGATCGTCGACAGCCflGTATCTTGGCGCTGACCmCTCGTGGCTGCAmCACCTACTNTNTGATnGCTGGCGN1CGA
CATCAGcTCTGCAAGAAGAAAGAGAGCATGATCGGCATGTCCACCAGCAGTGCTCTGGCGCTGGTCAGCACCGGGNTCTCITCCTGI1TGCTGCTGGACGACA
NACAGCTCTGTGNCTGNGGCCGGGGCATGNAGCCCCTCNAGCTGGAAAGGAAAAGGNCTGAGATGACG
>‘000127a-093.scf’ came from CONTIG 79 at offset 0;
“E:\SEQUENCE\export\EST_db\000127a\000127a-093 scf” (57>347)
GCACGAGGACCCTGCCATCCTCTCACnCCCTGGAACACTGGCACCTCCCTGCCCCACTlGGTGGGCGGCGGGTCCTCATGAACCTAGTGGGGCTCGGTGCCCC
AGCCCGGGTCTGGCGGCCAGACAGAGACCCGGGACCGTCCAGCCCCTACCCTCTCCCACCTGCCnCCTCCTGAGGAGGGCTCCACflGGACGCCAFPAGAATG
GCGCCCCC7FI7AGCTGAGTAGCGGGAAACCTGAGCCCACCGGGCCAGTGGACACCTGCGGCCAGTGGTGCTCAAG
>‘000127a-094.scf’ came from CONTIG 80 at offset 0;
“E:\SEQUENCE\exportEST_db\000127a\000127a-5094scf” (51>346)
CTCGCACACAGGGTTAnG1TACCATCTTTCTAAKFI7CCATATATATGCGUAGTATACTGTAGGTGTTYfrCrffCTGGCrfAC1TCACTCTGTATAATAGGY
fCCAGThrCATCCATCTGAAGAACTGACAAATATArrCrFfITAATGGCTGAGTAATACTCCATTGTGTATATGTACCACAGCmCnATCCACATCTGCTGATG
GGCATCTAGGGCTCATGTCCTGGCTAATAAACAGTGCTGCGATGCATTCTACCAAGAGGGGAGGNNGGGG
>‘000127a-095.scf’ came from CONTIG 81 at offset 0;
“E:\SEQUENCE\export\EST_db\000127a\000127a-095scf” (51>350)
CGCACCCTCACCGAGCACTCCTACGCCGTGTCTGCCGTGTCTTCCGTGGCTGAGGCACAGAAGGCAGCCTGCCCGGGAACAGGACAGCAGCTCACCTGCGAAA
ACAGGGGGAGAACTCTGGCTGflGCAGCAAAACCGAAAACATGCCGAAAGCCAAAGACACTCTCCGTACCCCAGGITCAGATTCCACTCCAGAAAATATACCA
CCCCCTCCAGCAAGCAACTGGGAGGTGCGTCAAGAAGTGTCCGCAGCCAGCTGCAGCTCTFfTCCC7ITPCAAATCCCCACCACGAA
>‘000 127a-096.scf’ came from CONTIG 82 at offset 0;
“E:\SEQUENCE\exportEST_db\000127a\000127a-096scf” (50>473)
CTGACAflAAFGTACACCCAACCCYFAAAAGATATGnCTGAAGATGCCmGGTnGATAGGGGnGAAGGAGACCCTAAGTATTrAGGAmTAAAGTmTAGCCCCmA
GCATGTTGGCCTGPGCATGTAAGGGTGGGCAGAGGGGCAmACAACCTGATCTCTCCTCCCTGGGCnCTCCTNGTGCCAAGC1TGGTGGGTGGCNTAAAAGGGN
ACAGACATCTCCffCCATCGACCTGTACCCTCTGCTGGCCGCTCCTCCAAAGCTAAAGGTCCCTGCTGGACGCACTGCTCflGGACCCAATAGAGGAAACACA
CGTGACACGATCAACTCAGACCGCATCAGmAGTGAGCCTGGATCAGTCTTFJCAGTCTGGGA
>‘000128a-001.scf’ came from CONTIG 1 at offset 0;
“E:\SEQUENCE\expOtt\EST_db\000128a\000128a-001scf” (46>520)
AArrGGCACGAGGGCACTCCAGCTCAGCACATGACCCTGAGACCGCCGTCCACTGTGGATGACCAGGCGGGAGAGATAGCTGACAAGATCTACAACCTGGATI
TGGGTACACGAGCGGCAAGGAGGAGCAGGCCGCCTACAACACGCTGATGGAAGTCTCTGCCTCCATGCTGUCCGCGTCCAGCACCACTACCTCACACTATGAG
AAGCGGCGATffGTCTGGAGGAGCGAGGACGAGCTGGmAGIGGCCCACCTGATCCCTCGGCGGCTGGAGAGGTGAGCAGCCACTGGTCCAGCCTcCTGcGIGc
GccTATACAGAGCCGNGTGGACACCGTGCCCTACCTCmGCCGCACGAGGNAGGTCGGNCTGCGTATGNGNGGNTACAGCATCTCAGGACACCAAGCACGTGCG
AGAGAAAATAGTGTCATGGNCCGCACTACGGGAGGCAGAN
>‘000128a-002.scf’ came from CONTIG 2 at offset 0;
“E:\SEQUENCE\expOrt\EST_db\000128a\000128a-002scf” (49>547)
CGACCCCCHGTGThCATGTCCGCGGCGCTCTGCTCTGGACGGCGGATACCATGTGCGCCGGTCAGCGCTCGGCGGGCGCCCTGGCGGCGGCAGCCCCGCGCAC
AGAGTACGCCCTCCGCGCGCCCTCTGGCCGGCGGGGAGCCCCCCTGGCCTCCCTGCGGGGCAAGGAGCTGCTCAGAGCGGAGCATAGCTCTGAGGCACAACGG
AGCGGGACTACACCCAGIGTGACCTGCAGAGGGGflUCGACCCCGGGCCTGGACGAGCTCGGCTCGCCTGCACCAGGTGGGCATGAGGACGCCAGACGAGAGA
TCTGATTGCTGAGTACGCCGACAGCGNGGGTCGAGCCAACTAGCCGGGCGAGGJThGGAGGCTCGTCTCGCTTCTGGGAGTCGCCAGCAGGACAGCATGr2AT
ACGACTAGTATACGCCCGGGGCGIGTCTGATTGAATCTGGGCCAAGGGCCGGCC
>‘000128a-003.scf’ came from CONTIG 3 at offset 0;
“E:\SEQUENCE\export\EST_db\000128a\000128a-003scf” (52>478)
GCAGGAGGCThHT1TEEITrCCTCCTTGCTACTATAACGmCCCCAGGGATGCCGTGACCCCTGAGCGATGGGTGGACCATCCTGTCTGCAGAGACCCCCCTGC
GATGCCCGGGGCGCCAGCCCTGCATAGATGATGATCfCACACAAAGAGGAANrfGTAACnATACTCCGAGCGCTGGAYmGACATCCATGAGGG1T1CTG
>‘000128a-004.scf’ came from CONTIG 4 at offset 0;
“E:\SEQUENCE\expOrt\EST_db\000128a\000128a-004scf” (48>489)
CGGGrTTGAGTATAGCCGTrCTGGAAATCCCACCCGGAAGCTTGGAGCAGTGGCGGCGCTGGATGGGGCTAAGTACAGTrGGCCmGC1TCAGG1TrAGCAGCC
ACTGTGACCAACCCATCTCnAAAAGCAGGAGACCAGAYFAThrGTATGGATGATGTGNTATGGAGGTACCAGATACflCAGGCAGAnACACCAGAAACCNAAG
CTGNTGGNATGACCCCCACANACCCTAGCGAAGATGAGACAVGAAGCCTGCGCACATACGGNCCATANACATGNAGACATAT1GGGNTGNGGATACACmATGT
CAGCATATh7CAGCGCCYFGCTCTGGAGCGATAG
>‘000128a-005.scf’ came from CONTIG 5 at offset 0;
“E:\SEQUENCE\export\EST_db\000128a\000128a-005scf” (46>459)
TGGGCCCCCCTGGAYFGGCTGGACCCCCTGGCGAGTCTGGACGTGAGGGAGCTCCTGGTGCTGAAGGATCCCCTGGACGAGATGGITCTCCTGGCGCCAAGGG
TGACCGTGGTGAGACCGGCCCTGCTGGACCTCCTGGTGCTCCTGGCGCTCCCGGTGCCCCCGGCCCTGTCGGACCTGCCGGCAAGAGCGGAGATCGTGGTGAG
ACCGGTCCTGCTGGTCCTGCTGATCCCA1TFGGCCCCGNTGGTGCCCGNGGCCGCG1TNUCCCCAAGGCCCCGNGGAGACAAGGGAGAGACAGGCGAACAGGG
CGACAGTAGCATTAAGGGTCAGGNGGGTCTCTGGTCTCCAGGNTCCCCCGGCCCCTCCGCIFLTNCTGTGAGCAGGTCC1TTCGAGCTCTGTCTGCTGTCCCC
GCGC
>‘000128a-047.scf’ came from CONTIG 5 at offset 14;
“E:\SEQUENCE\export\EST_db\000128a\000128a-047scf” (50>548)
FTGGCTGGACCCCCTGGCGAGTCTGGACGTGAGGGAGCTCCTGGTGCTGAAGGATCCCCTGGACGAGATGGYTCTCCTGGCGCCAAGGGTGAGCGTGGTGAGA
CCGGCCCTGCTGGACCTCCTGGTGCTCCTGGCGCTCCCGGTGCCCCCGGCCCTGTCGGACCTGCCGACAAGAGCGGTGATCGTGGTGAGACGGGTCCTGCTGG
TCCTGCTGATCCCATFTGGCCCCGrJ7GGTGCCCGTGGGCCCGCTGGTACCCTITNNTCCCGTGGTGACAAGGGTGAGACAGGCGAATCAGGCGACAGTAGCA
TAANGGGGTCACGTGCTCTCTGGTCTCCAGGGTCCCNCCGCCCCTCCGCITNCTGTGAGCAGGTCCITNCGAGCTCTGTCTGCTGTCCCGCCGTCCCTGCTTG
CTGTTCTCCCGCAGATGACTCATGGCTCCAGCCCATCGTCCCTGCCTGAGCGATGGATGTGTGGCTGTCCTCGCCTCGACCCTGTCCAGN
>‘000128a-079.scf’ came from CONTIG 5 at offset 43;
“E:\SEQUENCE\export\EST_db\000128a\000128a-079scf” (49>648)
TGTGGAGCTCCTGGTGCTGAAGGATCCCCTGGACGAGATGGrITCTCCTGGCGCCAAGGGTGACCGTGGTGAGACCGGCCCTGCTGGACCTCCTGGTGCTCCT
GGCGCTCCCGGAGCCCACGGCCCTGTCGGACCTGCCGACAAGAGCGGTGATCGTGGTGAGACCGGTCCTGCTGATCCTGCTGATCCCATFJGGGCCCG7IITG
GTGCCCGTGGCCCCGCTGGACCCCAAGGCCCCCGAGGAGACAAGGGTGAGTNCYI7CGAACAGGGCGACAGATGCKITAATGGTCACCGTGGCTICTCTGATC
TCAGGGTACCACCCGGCCCTCCNGGCTCTCCTGTGAGCAAGGTCCTANCGAGCCTCTGTCTGCTGGGCCCGCGTCCCCTGCTTGCTGCTCTCCNGCAAATGAC
TCATGTCTCCAGCCCATCGTCCCTGGCCTGAGTCCCTGTGTGCTGTCTGCTGTCTCCGCCTCTGACCCTGTCCCAGTCTCCACGGGCTCACTGACTCTCCCAN
CACTCAAAGTCGAGTGGCGTI7ACGGCGGTGCATGGNCGGACGACGCGGGCCCACTCACGNGCAAGAAAACGGCCCGGCCGAACCCCCCCC
>‘000128a-006.scf’ came from CONTIG 6 at offset 0;
“E:\SEQUENCE\export\EST_db\000128a\000128a-5006scf” (51>507)
GCACGATGTGGAGCTCCTGGTGCTGAAGGATCCCCTGGACGAGATGGTTCTCCTGGCGCCAAGGGTGACCGTGGTGAGACCGGCCCTGCTGGACCTCCTGGTG
CTCCTGGCGCTCCCGGAGCCCACGGCCCTGTCGGACCTGCCGACAAGAGCGGTGATCGTGGTGAGACCGGTCCTGCTGATCCTGCTGATCCCATTGGGCCCGT
TGGTGCCCGTGGCCCCGCTGGACCCCAAGGCCCCCGAGGAGACAAGGGTGAGTNCTTCGAACAGGGCGACAGATGCKITAATGGTCACCGTGGCTTCTCTGAT
CTCAGGGTACCACCCGGCCCTCCNGGCTCTCCTGTGAGCAAGGTCCTANCGAGCCTCTGTCTGCTGGGCCCGCGTCCCCTGCTTGCTGCTCTCCNGCAAATGA
CTCATGTCTCCAGCCCATCGTCCCTGGCCTGAGTCCCTGTGTGCTGTCTGCTGTCTCCGCCTCTGACCCTGTCCCAGTCTCCACGGGCTCACTGACTCTCCCA
NCACTCAAAGTCGAGTGGCGTTACGGCGGTGCATGGNCGGACGACGCGGGCCCACTCACGNGCAAGAAAACGGCCCGGCCGAACCCCCCCC
>‘000128a-006.scf’ came from CONTIG 6 at offset 43;
“E:\SEQUENCE\export\EST_db\000128a\000128a-006.scf” (51>507)
GCACGAGGCAGAGTCCTGGTGACGATGCCGCGTTCGCCCCCTCAGTCCACTCCGCCAGCCCTCCACCGCTGCGCCCCGCCAGCCCGCCCCTTTCCTGTGCCAG
GCACTGACAGGCACCATGCCCCACCAATACCCAGCACTCACCCCGGAGCAGAAGAAGGAGCTCTGTGACATCGCTCACCGGATTGTGGCTCCGGGCAGGGCAT
CCTGACCGCAGATGAGTCCACCGGGAGCATTGCCAAGCGACTGCAGACTNTTTACACCGAGAACACTNGAGAGAACCGGCGCTACTACCGCCAACTGCTGCTG
ACTGCCGATGACCGCGAGAATCCCTGCATCGGGGCGTCATCCTCTTCACGAGACGCTGTACCAGAAGCCGATGATGGGNCGNCCTTNCCCCCAGTTATAAAGC
CCAGGCGNGGGGNGGGCAATAAGGAGAAAAGGGGGGGGNCCCTG
>‘000128a-007.scf’ came from CONTIG 7 at offset 0;
“E:\SEQUENCE\export\EST_db\000128a\000128a-007.scf” (47>316)
TCGGTGGGATCTCTFTACGTCCTCTGGACTGGAGGCCGAGCCCCCGCCCGCGGCCGAGCCCCGCGCCCGGCGTCTCCGCCCGGTGTGCTCTCCGCAGTGTTCC
TGGGCTTGGGAAGACCTCGGGGAACATGGCGAGGCAGCGGTGGTTTAACGGGAAGGACGGTGACTGTTAGCCTGTGAACGAAAGCGAGAGTGAGCCGCCTCAC
GCTCCGGACCAAGAGTGATCTTGAACTTGAGGCTGCTACTAGATTATTGCGCACACCTGCTNTC
>‘000128a-008.scf’ came from CONTIG 8 at offset 0;
“E:\SEQUENCE\export\EST_db\000128a\000128a-008.scf” (45>464)
TAAAAAAAGGAAGCTTGGTCCACTTGCTTGAAGACCCACGTGGGGGTAAGTCCTTTTTCTGCCCATTGGGCTTATGACACCCCAGCACTGCCCTTTCTGCTCC
TTTCTCCATGCCTTCTTAGGGCCTCCCCTCCACTGGTCCCCAAATCTAAGTCTCCCCAAAAGACACAGGAAACAATGCATTGTCTGCCCAGCAACCAAAGGCA
ATGCTGAAACACCCAAGAGGCCCCCACACTCCCAGCCCACTTCCYfCACCCAGAACCTCNTNTTCTGGGGGACCTGGAGTGCTCAGACTGCCANAGAAGCTTT
ACCATCTGGCATCCCTGGGGCCCGGGGACATTCCCCTCTITGTTITGAGGGAAGCATGCCAGGGGGACACTGGCCCThCATCACAGNTGGAGGAANGCAGAAG
GGNNCAGANG
>‘000128a-010.scf’ came from CONTIG 9 at offset 0;
“E:\SEQUENCE\export\EST_db\000128a\000128a-010.scf” (316>320)
TTGAA
>‘000128a-012.scf’ came from CONTIG 10 at offset 0;
“E:\SEQUENCE\export\EST_db\000128a\000128a-012.scf” (43>539)
TCTCAACTCCATGATTTGTGGAGAGAAGGAACGAATCTTTGATGGGAAAAACAGAGAAAAATCCCTCTTTTTTCCCCCCCATTCTTATAAAATCTTCCTTTCA
TCAGTTTTTTATAAAAGTTGCTTTTTTCATTTGCAAGTTGTACAGTTTACCACTACTGCTGCTGCTACTGCTAAGACGCTTCAGTCGAGTCCGACTCTGTGCG
ACCCCATAGACGGCAGCCCACCAGGCTCCCCTGTCCCCGGGACTCTAGGCATTAGAAATTNTTTTTATCTGTGAGGTATTATAAGTCATTAACTATTCCCTTT
CGATAATAGATGTAAGCAACTATTAAAAATATTAGTTAGTCACAGATTTGACTGAAAATCTCTTACAAGAAGAGGAACAGAAATAANGAGCAGNTNGAATGGN
GCTGNNACATGGAAACATCAAGAAAGGNGACACACTGTTTTTTTTTCTATCTCTATGTTTAATTTAGNAAAACAACTNTGATG
>‘000128a-017.scf’ came from CONTIG 11 at offset 0;
“E:\SEQUENCE\export\EST_db\000128a\000128a-017.scf” (100>532)
AGGAAGGTCGGGGCTGGGGGGGGGAAGGCGCGGGGGTGGGGGGGGGAGGAGGGGAAGGGGAGGGGGGGGGGGGCGAGGAGGGGAGGGGGGGAAAAGGGGGAGG
GGAAGGGAAAGAAAGGAGGGGGGAGAAGAAAAGAAAAGAGAAAGGGGGGGAATTTGAGAAGGAGGGGAAAAAAAGGGGAAAAGGGAAAAAAGGGACAAAGGAA
AAGGGGGGGGGGGCGAAAAAAAAAACGGGAAGGGGCCGAACGAAGGAGGAAAAAACAAAAGGGGAATTAAAGCAGAGAGGGAAAGGGGGGACTACGGGACAAA
GTTGGGAATGAAGGAACAAAGGCCAAAAGACGCGCCCCAGGGGCCGGGAAAAAAACAAAAACGAGATAGCGAACGGGAAAGGAAAAAAAGATACGATTAAAAA
CCCCCCCCCCAAATACGGGAA
>‘000128a-018.scf’ came from CONTIG 12 at offset 0;
“E:\SEQUENCE\export\EST_db\000128a\000128a-018.scf” (60>253)
AAATATTAAGAACGTAAGAAAAGCTCACTATATAGAAAATGCTATACCCGGAACAAAACAATGGGGGAAATGCTGGTGGAGGAGACAAAAGGGAAAAGCAAAG
GCAAAATGGCAGGGAGGAAGGGAGAGGAAAAGAAACCGGAAGGGCGGGAAAGGCAGGGAGAACACCGCGGGTGAAGGGCGGGATAGAGATG
>‘000128a-019.scf’ came from CONTIG 13 at offset 0;
“E:\SEQUENCE\export\EST_db\000128a\000128a-019.scf” (99>348)
AATAAGAAATAGGCTCCTTGTAAGTCTTTATGAAAACGGAAACTTAGAGGGTAAGAGGACTGGTGGGGAATACAAGTTGGTTGGGATPAAGAGATGATGAGCA
GGAGAAAGAAAAATTATGACCATATATGGAATGGAGGAAGAGGAGGGAATATGGGAGAAAGAAAAGGGAAAGGGACGGACGGCGGCTTTGAGGGAAGAAACGC
GGTCTTCCAAGAAATAGAATAAAAGAAAAAGAAGAGGAGGGGGG
>‘000128a-020.scf’ came from CONTIG 14 at offset 0;
“E:\SEQUENCE\export\EST_db\000128a\000128a-020.scf” (1>365)
AGGGGGGTTAATAGACCCGCGCGGGGCCGGCGCTGATACACCACCTGGGGGAACCCTTCCGCGCAGAGAAAAGACCTGGACCCAGGCAAGAGGGACAGAGACA
TCCAGCCGAAGAAGGGCGCCTGAGAAAACATATATGGGAACGACAAAAGAGACACTATGAGGCAAAGGGCCTGGGGATCCATTGTAGGAGGGATCCAAAGCCA
ATGCCTCAGCGCGGAAGAACCTGGCCTTTGGAACCAAAGGCCACACACCGCGAAGGAGCCAACAAAAAGGGGAGGGGGCCTCTTGGAACGAAAAGGCTGCCCG
AAAAAAATAATTGAATTGGGGGGGAAAAATGGCAAAAAACCTAAGGGGGTTGGCCGC
>‘000128a-021.scf’ came from CONTIG 15 at offset 0;
“E:\SEQUENCE\export\EST_db\000128a\000128a-021.scf” (3>262)
GCGTGGGCGTTTAACTCGGTCCCGTCTCGCGTTCCGGCCTTFGATATCTTTCATCTTCGTTGGGTAACCGATCTCGCGCAGACTGAAAATACCCTGGGGCACC
CTATGGCCACAAGCTTGGTGACCCAAACGCTACCACTCGCCAGTTGCCGGACATGATATGTGCGCCTGTGAGATTAGAGAGATAAGGGGAAGTGAGAGAGAGA
GAAGAAGAGGGGGGGGGGGAGGGGGGGGGGGGTTTTGGGGGGGGGGGGAGGGGG
>‘000128a-022.scf’ came from CONTIG 16 at offset 0;
“E:\SEQUENCE\export\EST_db\000128a\000128a-022.scf” (411>415)
CAGAA
>‘000128a-023.scf’ came from CONTIG 17 at offset 0;
“E:\SEQUENCE\export\EST_db\000128a\000128a-023.scf” (336>462)
GGGGCGCGCGGGCTCTGGTGGGCCGGGAGGAGGGGGTAGGGGGGCACGGGGGGGGGGAGCTTTGGCAGGATAGAGGCCCCAAAAGGAGAGGGAACCCGCCGCC
CCGGGGGGGGCAGGGCCTGGGCGG
>‘000128a-024.scf’ came from CONTIG 18 at offset 0;
“E:\SEQUENCE\export\EST_db\000128a\000128a-024.scf” (29>467)
GGCTGTGTGGACTTACAGGACTCTGAGGGCGCTCCTCATGAATGAAAATTAAGCAACACGGAGGGAAGGGAAAGCGCACCACTTCATATAACTTAAACAGTTG
TGAGCAACCTGGCTAGCCTGGCTGGCGCTATGAGACGGGAGCTGGGCACGAAGAGCCACTGGTCGTAAGAAAAGGAAGTCGGGGAGTTTGAGGCGCAAAAAAA
AAGCCCGCAGCCCCCGGACCGTCAAGCCAAATGGGGCCTGGGGACCAGGGGACCAAGGGACGACCAGCGGGACAGGGCCAGAGGAACGTAAGAAGGGCAACGC
AAAGGGCAAGGCCGCGCACCGCTAAACCACACCGCACGCCGCCCAATAAAACATGGGGTAAAGCGCGCCTTTTGTCCCCGCCCCCCCCTCATTTTCTGGGGGT
CGGTTTTGTAAATTAAAAAAATTTTT
>‘000128a-025.scf’ came from CONTIG 19 at offset 0;
“E:\SEQUENCE\export\EST_db\000128a\000128a-025.scf” (1>247)
CACGGGTGCCCTTTAATATGGTCCCCCGGGCTGCTGCTGATTTCTTTTTCTTTTGGTAGGCTGATGCACTTTGTTTTGCCAGTCAATGGCAAGATAAAACTAA
GTGAGAAAGAATGCAAGGGATAAAAAAAATTTGCATAGGGCATGTGAAAATGTCTCCATTTTTGGTTGTTGGGGTAAGATGGGGTGGTGTGGGGTGAAGTAGG
GGATGGGGTTGAGGGGAGGAGGCAGGTGGTGAAGGGGAGG
>‘000128a-026.scf’ came from CONTIG 12 at offset 0;
“E:\SEQUENCE\export\EST_db\000128a\000128a-026.scf” (1>587)
CAGGGGCGTCGTITACTTTGATTCCCGGCTGCGGAATTCGCACGAGATTGATCTACCAGCTCCAATTAAAACTGCAGGCATAAGCCCAAAATGTGACTAATAT
AAAAGCCCTCAGGGATGTAATPAAATTATCCCAGTGCCTTTFFTAGTTTTTAATATCAAGATTGAATCTGTTATGTAGAGGCACCAAAATGAATGTCATGCTG
GNAGTCTGTATGCATGACTCAAATATACCTAATACAATGTCAGAGNTGTATAAGCCAGCAGAATTTATTTTATAGCAATTCAGTATCTGTTTACCTACAGGNT
CGGGGTTGGGGAGTATATTATGAAGAATCAGATTAGAACTGACTACTAAGAGACTATGGATCCACTATTAGCCACTCAATATAGACCACGCTAGACCCNAGAG
AGCTATACAAAACAAAATCTATTCNCCCTACTTATTTAAGTCTCCTTTATACATACCAGGCCTACTGCTAGACGAAAGTGAGGGGCCTGGGAGGCACACTGAC
TCCCTTCCTGCCCAGCATAGGAATAAGAGTCAAAGAGAGATACTCACACCTTTCCCCTTCGGGCTAAAGCGCG
>‘000128a-027.scf’ came from CONTIG 21 at offset 0;
“E:\SEQUENCE\export\EST_db\000128a\000128a-027.scf” (1>587)
CGCTGCATGGCACATGCCGCCACAATGCCACACTATACGAT
>‘000128a-028.scf’ came from CONTIG 22 at offset 0;
“E:\SEQUENCE\export\EST_db\000128a\000128a-028.scf” (1>525)
CACGGGGGGCGTCTAAACTAGGGATCCCCCGGCTCAGAACTGCACGAGTAGAACAAACCACACGTTAATACTGCTGTGTGATATGAAGCCATAGATCTTTCTA
CTATGCTTTGGATACGACCTACGCCCAGTGCAGAGTCGGGGGTGCGTGCTTACAGACCAGATGATGTATGATTTGCGCTGGAGCATGCGAGAAGATAAATAAC
TACCCTAGCGCGAAGAGGCTCTATAGCATAAGTATGCGAGAACATGCGGACCAGTTCTGAAGTAGAATAAGAGCATGCTGGAGCTGTTTCTTGCAGTGGACAC
AGTGAAAGACACATAGGCTGACAACAGATGTGCATGACAGATGAGAGACGACTCACCGAGGAAGACACAAAAAATAAGGAAAGGGCGCAGAAAGTGAGCACTC
AGAGGTAACTGGCGACATGGGACTTGGCAATCAGCGCAGGAGACGGGCCTACTCACGCTGGCTTTGCACACCTAATTGAGAAAGGAGCGAGGGCGACTGGGGG
TGGGGGTGTGTG
>‘000128a-029.scf’ came from CONTIG 23 at offset 0;
“E:\SEQUENCE\export\EST_db\000128a\000128a-029.scf” (1>363)
AGGGGCTCTAACAGTCCCGCTAGATGCCAGCATCTCTCAGAAGCGCAGGTCGCACTCGGTAGGAGCGCGCCAGGCAGGCGGGGAGCGAGGGACGAGAGACGAC
AGAAGGGCCCTAACCGAAACTGTGCCGCCCAACTGGCAGCTGGAGGAAACGTACGCCAGTGAGACCCAAGTTTCCCTATCAGAGACGGCAGAACACTACGGTG
GCGAGAGCCCAGCCGAGCGCCAAGCCTCGAGGCGGGAGGAGCAGTTGGCTTTGGGCTGCTGTAAGCAGTGACCACACGCGTTAAAGTCTCAGACCCCAAAAAA
AAAAAAAAAACGGGCCGCCAGCTAGAGTTTAACGCGGTAGGGAGGACGGTCCTAC
>‘000128a-031.scf’ came from CONTIG 24 at offset 0;
“E:\SEQUENCE\export\EST_db\000128a\000128a-031.scf” (1>431)
AGGGGCTTAACTGTCCCGTAGCTGCTGGCCGGGTGAGACTTTTGCTACCCGATCAACCGGCGACGGGCGGTCGCTCCGGACTCGCAGCGGGGAGGGTCACTAC
TCTGCGACGAGGGAAGAGCGGCGTGTCCGGAGCAGAGGAAAAATTGACTAATGAACGATAGAGCTCTAGTTTTGCTGGACCCCGAGAACGACACTCCGCCACC
ACGGATCCCAGGACTCTGACGAACCAAAAGCATGATGAGAGACGAAGTAAAAGCTTTGTGGGGAGGAGGGGAACACCTATCGACACGACCCCGTGACAAGGGA
TGGGGCACCTAGATGAAGAAAAATACAGTGAGTTATGAGGAAAAAACTGAACTTGTAATTCCACTGAGGGGGTAAATAATCAGGATGGCCTCCGCGCTCTTGC
GAAACCGCTTTCTTTAAAG
>‘000128a-032.scf’ came from CONTIG 25 at offset 0;
“E:\SEQUENCE\export\EST_db\000128a\000128a-032.scf” (178>503)
GTCGCGCAGAACGCAGAACAGCCCCGAGATCACCCAGACTGCCGAACGAGCCAGACTTGGGGTGCCCCTCGAAAGCAGACAGAGCTGAACTAAAGGGCCCTTT
TTGGACGGGAACCACGCCTGATTTCACAAAACAAGCGGCAATAGAGAGCTGTATCCCACCTACTTATAGCAAACCAAGGAGAAGGCCCAAAATTTTCAAAACA
AAGAGAAAGACTGGGATTTGCCCAAAGTAGGAGGAGCGAACCGGATCCCCTCAATGAATGCACAGATCAATTACTAAAGCTTCCCGGCAAAAACTGTGCCGCC
TCAACATTAGAACGA
>‘000128a-034.scf’ came from CONTIG 26 at offset 0;
“E:\SEQUENCE\export\EST_db\000128a\000128a-034.scf” (1>120)
CAGGGGGCGTTAACAGGATCCGGCTGAGGAAAAGTCATGTACGCATGCTACCAGGAAAGAAATCAAGGGACGGCACTTGAGAAGCCTITATGGGGGGTGAGGA
GGGGGGGGGGGGGGGGG
>‘000128a-035.scf’ came from CONTIG 27 at offset 0;
“E:\SEQUENCE\export\EST_db\000128a\000128a-035.scf” (54>235)
GCACGAGGCTGTFfTCATLTAGCTGCTTTATATGGAGAAGAGAGAAATTCTGTGTTTCCAGATCCCAACAAGGGGTGCATAGAGTCTGAAGACATTCTTTTCT
ATTTTCCTAATCCCCCTTCTGCTGTCTCTGGGAGTGCCTACTGGCACAGAGGCAAGGTATTTGCAGAGAACAGAAANGT
>‘000128a-036.scf’ came from CONTIG 28 at offset 0;
“E:\SEQUENCE\export\EST_db\000128a\000128a-036.scf” (43>286)
CCCGCGTCTATTTCAGAGCCCCCCGGGGCTGCGGCGAGGCCCGGGCGCGCGGACGAGAGGGCCCATGAGGCGGCAGGGAAGGTCACGGTCAAGTACGACCGCA
AGTAGCTACGGAAGCGCCTCAACCTGGAAGAGTGGATCCTGGAGCAGCTCACTCGCCTCTACGACTGCCAGGAAGAGGAGATCCCAGAGCTGGAAATCGACGT
GGATGAACTCCTGGACATGGAAAGCGATGATACCCGG
>‘000128a-037.scf’ came from CONTIG 29 at offset 0;
“E:\SEQUENCE\export\EST_db\000128a\000128a-037.scf” (1>281)
CACGGGCGCATAGGTCCCGGCTGAGGTTGGCTGTTATTGGATTGTGAAATGCTTACTACGAAAATCTGAAGCTAGCCAACGATGATTGAAATCACAAGTGGGA
CAGCAGGAGGAGTATCAGCTCTCTGAAACTCTCAGCAGCTCGCGAGCCGCATAACACTGCGACGATAACAGAGACGCTTGTTCAGTCCTNAGAATCACAAACC
AGCAGATGAACTAAAGACGCAGGACAACTATTCATACCCATGTTGGAAACATGACTTCAAAAAATAAATAAT
>‘000128a-038.scf’ came from CONTIG 30 at offset 0;
“E:\SEQUENCE\export\EST_db\000128a\000128a-038.scf” (8>426)
GTTAATAGATCCGGCTGAGTGAAATGTGACACCATTCACTCAAAAAGGATTGACCCTAACCAGAGGACCTACTGCAACGAGCATCACTGGCCCAGCTTGAGCA
GGGACAGAGAGAGTCAGAGCTGGCCCTGTACGCAAAGGAAGCAGCTCAGGTAGCTCGCAATCAGTGAGAGAGTCGGACAATGAAACAGATTNCGCACYFGTTG
CCGCGGAGACGACAGGCACGCACNGCTGTGCAGCAGCCGAGACGATACTCAACCTACAAAACGTCAAAGTGAAGGAATGTGAGAAGAAGGGCAGCCGTGAACA
CACGACGCTTGCTAACAGAACAAAACCCACTGCCAACAGCCGACGGCCAGCCTAAACCAGAGACTAAAGAAAAAAAGACTACACAACCATGGGGATTGAAAGG
CGCGAGG
>‘000128a-039.scf’ came from CONTIG 31 at offset 0;
“E:\SEQUENCE\export\EST_db\000128a\000128a-039.scf” (48>549)
GGAAATTGCCCCAGATGCGTCCTTCATTGATGACGAAGCATTTAAGCGGCTGCAGGGCAAAAGGAACCGAGGGAGGGAGGAGATCAACTGTGTGGAGATCAAA
GGTGACGACCAGCTCAGTGGGGGCCCAGCAGTGGATGACCAAGTCATTGACAGAAGAAGAAACCATGAAATCATTCAGCAAAAAGAAAGGTGAGCAGCCAACA
GGCCAGCAGCGGCGGAAACACCAGATTACGTACCTGATTCATCAGGCTAAGGAGGTTNTNCTGGAGCTGAAGAAACACTGGTCGGGAGAACAAGCTCAGCCGC
CGGCAGACCCAAAGCAAATANNCGATCTAGGGCTCTGGACTGACTGCTCTGGATCNCTGCAAGCCACTGGCTCGGGCCCAGCTCGNCTCTGGACCCAGCTGAT
CGAGCCAGATCTCTTTCCTGAGACCNAGCCTCGCTCTGGAGATGACTATTGAAGATTFITTCTACAAAGTAAAAATCACTCTTCTGGCTGGA
>‘000128a-040.scf’ came from CONTIG 32 at offset 0;
“E:\SEQUENCE\export\EST_db\000128a\000128a-040.scf” (47>605)
TTTTTTTTTTTTGCAACAGTAACCCCCTGTCCAGAGTCTGACTGTAGCTGAACTGTTCAGACTGAGGAATGGAGCAGGCTGTGGGCGCACGCCTGATCCCTCC
TGGGCGAGCGCCCCCACCCTCAGGGAACAGGCTGGAGCCAGACCAGCTCACTGCTTGCTGGCCACCACACACTAGCCATACAGAACATCATCATTATCTTCTG
AGTACACACTGCCACCTGTGCCACCGCCACTGCCCTGACGGGGACAGCTCATTCTNGTTACTGAGGGAATCTGAGCTGGCAAGCCTCGACTTGCTGAAGGGCT
GAGCAACATTCATACTGCGGAGTCATTATACTGACAGACGGCGGCAAGCGCATAGCTCTCAAGGATCCGCGGATCTAGCCTGATCATCTTTTATTCATGCTGA
CGGTGTTGCGTCCGTCTGCCATTACTTGATGATCAGAGGCACTGAGGCGTGCAATTGGCAGCATCAAAACCTATCTTTACAGACTATCATCTGCATGGCTGGC
GTGCTGAAGAACGGCTCTTGAAGGAAAAGGAAAGGCGGGAGAGG
>‘000128a-041.scf’ came from CONTIG 33 at offset 0;
“E:\SEQUENCE\export\EST_db\000128a\000128a-041.scf” (14>537)
TTCTATACTATGGATCCCCGGGCTGCGGTTTTTTTTTTTTTTTTTTTTTGAGGTTTAGAATCTGCTTTTATTATGAATATAAAATATACATACAACATAATAC
ACATTTACACKTTTACAATTTGCAGTTAGTTTCACTTTTTTGAGCACGTTTGGTTCTGCACGGCACAGTGGCCCATGTTCCTLTCCACTTTACACTCAAGACA
TTCTCTTCACCTTGATACATACTTGGGAAGAAATACCAGGCCGAGCGTCANATGGCGAGCTTCACTGTCTTCCCAGAATCACTTNTCTTTCTGATTCCCCTTT
GCTGCTACAGGCTTTCATGGCGCTAAACTCCAGCATAAAAAATGCAGAGAAAAGAGGCTGCAGAATCCCCTTCGNCTTTCAAATCACCCTGAAACATCCCACC
TGACGAGGNGAACATCATAGCATGCTGGATAACATAGCTAAAAACTACTACAGGAGCTTGAAGGACACATACAACTAACTGTCACCATGATGATACAAGGATC
GAATCGGGTAT
>‘000128a-042.scf’ came from CONTIG 34 at offset 0;
“E:\SEQUENCE\export\EST_db\000128a\000128a-042.scf” (48>687)
TAATTCGGCACGAGGGTTGCTTTGGGAGCTCTCCAGCACCTTCAGGAAAGGTACAAGAAATTNTTAACAAATCATTTTAAAATGCAATTTAAAATTCTCAATA
AAAGTGATTTTTTAAAGACATTTATTTATGGCTGAGCTGGGTCTTCTTCGCTGCGCACGGGCTFFCGTCTAGTTGCTGTGCAAGGGCGTCTCATTGCAGAGAC
TTCTCTTGTTCAGAGCACAGGCTCTAGACGCGGAGCTGCAGTACTTTNCGCACANCNNNTCAGNAGNTGTAGCTCCCAGACTGTAGAGGGCAGACTCAGTAGA
TGGGGAGGGGCACAGCTTAGTGCTCTGAGGCATGTGAGAATCTCCAGATCAAGAATCAACCTGTGTGTCCTGCATTGCAGGCAGATTACACTGAGCCTCAGGN
AAGCCAAAGNGATATTAATATTAAATACTCTGATTGATATGAATTTTTTTTTCCTGTAATCATCAGAAAAAGGGCGGCAACAAAAACTACGCCAAGCACACTG
CTCATCTCATTACTTAAAACANAACTACGTCNAAGAAGAGAGAACTTCTATACAGATTACCAGACTTTAAAAATAGTGGGCTGGAATTGGTTTATGGGAATGG
AAAAAATTTGTTTCCCGG
>‘000128a-043.scf’ came from CONTIG 35 at offset 0;
“E:\SEQUENCE\export\EST_db\000128a\000128a-043.scf” (334>638)
AGAAGACAAGGAAACAGAGAAGAACAAGGGCACCCAAACACACACACCCCGGGCCCTGCGGTTCGGACGGATGGTGCCGGCCTGAAGAACTCCCAGAGGTCCG
AAGGGACGGAAGGGGGAAAAGGGTTGGGTGGAAACCCCCCCCTITCCGGCGGCGATCTGGACCCThTITACCCAGAGGGGGCTCGCCCTAAGAGGTTTATGTA
GGCCGGGGGGCCGAATAACTGCAAAGAAATTAAGTGCAGAGTTTTAAAGTGAAAATTTTTTGAACCCTACCTCCTAATTTGTTTTTCTGGGGGGGGTTTAG
>‘000128a-044.scf’ came from CONTIG 36 at offset 0;
“E:\SEQUENCE\export\EST_db\000128a\000128a-044.scf” (1>359)
CGGGGGGTTATAGTCCGCTAGTGGCGCGGGTGGTTGTGAGAGGGTGTAATGTAGACAGCTATTAGAATACAAGGAATTAAAAAGGAAGGCAAAATGATATATG
TGAAGATGTAATCAATAAAAAAGACACACACTAATCAAGTGTGGTAGACAAATATATTGTAATGCTATGCGATAGAGAATATGATTTTGGCAGCTTTGCTACG
ACTCCAATATGNGGAAGTGGNCGAAATTTGTGGGTTTGACGGCGTGTGGCGCGCTCTATAGTGGTGAAATTTGAGCAGGAGGAGGAGATCAGGAAGTGGGCAC
GGCTTCTGTCGAGCGATGGAATCTCGATAGGTTGGGCTAGGGCGGAGGGT
>‘000128a-045.scf’ came from CONTIG 37 at offset 0;
“E:\SEQUENCE\export\EST_db\000128a\000128a-045.scf” (50>308)
GCACGAGGGGCAGAGGTGCAACTTTCTTCGGGCGGCCCGAATCCGGGTTCATCCGACACCAGCCGCCTCCACCATGCCGCCTAAGTTCGACCCCAACGAGATA
AAAGGCGGAGCGTGCTTTAGATGTGGGCCGGGGCTTCGGGATGCGGCATCCCTCCCCGTATTNCGTCCGGGCCGCGGCGCCCGCCGCTATGGGCTTTCCCACG
TCGGGCCTCAAGGCCGCTGCCTCCTAAGGGCCCTGCCTCTGGTGCTGNNNNNA
>‘000128a-046.scf’ came from CONTIG 38 at offset 0;
“E:\SEQUENCE\export\EST_db\000128a\000128a-046.scf” (54>610)
GCACGAGGGCTCCCCCCTCCCCCCGAGCGCCGCTCTGGCCGCACTGCGCTCGCCCTGAGCTCCGGGCTCCTGCTAAGCCAGCGCCGCTGTCGCCTCCCTCCAG
TCGCCATCATGATCATCTACCGGGACCTCATTAGCCATGACGAGATGTTCTCCGACATCTACAAGATCCGGGAGGTCGCGGACGGGCTGTGTCTGGAGGTGGA
GGNGAAGATGATCAGTAGGACAGATGATAACATCGATGACTCGCTCNTTNATGGAAATGCCTCCGCTGAAGGCCCCGAGGGCGAAGGTACCGAAAGCACAGTA
ATCACTGGTGTCGATATTGTCATGAACCATCACTTTGCAGGANACCAGCTTCACAAAGAAAGCTACAAGAANGTACATCAAGATTACTGAAGNCAATCAAGGG
AACTTGAGAACAGAGACAGAAGAGAANACCTTTATGACAGGGNCTGCGAACAATCAGCCATCCTTGCTATTJCAAACATCAGTCTTATGTGAAAACTGATNCA
ATGCATGGTGCTTGTGACTACGGAGAGGGGNACCCATATGATTT
>‘000128a-048.scf’ came from CONTIG 39 at offset 0;
“E:\SEQUENCE\export\EST_db\000128a\000128a-048.scf” (52>580)
GCACGAGGGTGCTTGAGTTCCGTCTGCGGCCAAGGAGCTCGTCCCCCACCTACCCCCACCCTCTTCCTCTCTCCTCCCAATCCCAGGCGTTCCCGACACTCTA
GGCGTCAGGAGGCACGCCGACCAGCGTTCCGGCTGGGGAAAGGGTGGCGAGCGGGACCGCCCGACGTTGGGGTTCTAGTGTGAGACGCAGGTGCGGTCGGTNT
CAGGAATTAGGACATCGGCTGGGCCTGAAACTCGCTGGGCATGCAGAAATGTCCCTCGNCCGCGGAGACTGGCTGTCTCACGGAGCGAGGGACGTGCATGTAC
CCCCGCCTCAGAAAGCGGCCNGCTGGCAGCCTCATGGAGGTGGNNTGTGAGCAGTGNGAATAACACGCCANAAAGCTAGCAGCTGGCGACAAAGTCAAATCCT
CTGCGNNCCACCCACCCAGCATCAGCAACTACGCCAGGGACAGNGCCTGCCCATCTGNGTCTCGTCCTCCGACCCCCTCCCCCCCCCTCTCTGCTACTCCCGN
GCCACGNATGTG
>‘000128a-049.scf’ came from CONTIG 40 at offset 0;
“E:\SEQUENCE\export\EST_db\000128a\000128a-049.scf” (48>538)
TTTCTTGGAACTAAGAAGGACTTACCTGACATTGGCCTCATTCTGGCCTTCACTTGTTCATAAGAATCATGGAACCAGAGTTTGAGTTAAGAAACTTGGAAAA
AGCAGCTGAAAACATCTCAGAGTCCACTTAACAATTTAAAAATTCCACTTAAGATGCTAAATAATCCATTGCTTATGTAGCAACTCAACGATGTTCTCAGGNT
CCCCAGTTCTTTCTGTCTTTCCCCTCTGNGATCCTTATTGTATGGGGCTTTGNNNNCACGGNNTTCTCAGNGAGTACATATAGGCTGNTGCTGCNTCAGACAT
TTNCATCTCTCTCAAGACTGGNAGTAGGNAGCAAAACCTTNGTCTTTCAGTCTCTGTTTTTATTTGGAAGAANAATCTCTTCTCAAAGTCCTCACATTTNCTT
NATCTCATACATAGGATTTTCCCTACAATCTGATCACAGGAGCTGAAACGACAGGANGGNAGTGATGCTGGTAGACCATG
>‘000128a-050.scf’ came from CONTIG 40 at offset 0;
“E:\SEQUENCE\export\EST_db\000128a\000128a-050.scf” (8>563)
GCGTTAACTAGGGTCCCCGGCGCGGGGTCTGTGCTTACAGGCTGGACGGACAGACCCAGGACGCCCCTTGCTCCAGCCTCCGACCACCCTCCAACCTTTTTTC
CAGTCGCAACCTTCGGAGTCAGCCACTCAGCTGTCCGCGATCACCGGGACCAGCCACCTTTTTTAATCTCTTATTATFACCGACCAATCATGAGCTGCCAGAT
TCGTCAGAATTATTCTACGAGGTGGTAGGCGNNCGTCACCGCCTGGTTAACATGCATCTGCNNNNCTCTACACCTACCTCTCTCTGGGCTTCTATTTCGACGC
GACGATGTGGCCTGGAGGGTGGGGGTCACTTTTTCGCGAATGGNCCAGAGAAGCGCGAGGCCGGAACGCTCTTGAAACTGCAAACCAGCGGGCGGCCGGCCCT
CTCTGGGAGGNCAAAACCATCTAGAGANGGGGGTAAACCAGACCTAGGAGCCGCTCTCGANAGAAAACTGATANCTNTGGACGCAGGCTGGCTTTGCCCGGAA
CCCAATTGGACTCTGAAACATCTAATGGAAGAANCAAA
>‘000128a-051.scf’ came from CONTIG 42 at offset 0;
“E:\SEQUENCE\export\EST_db\000128a\000128a-051.scf” (8>563)
CCACCGTGTGGCGCCGCTCTATACTATGGATCCCCGGGCTGCAGGCTTCAGTGGCAGCCAGTGCAGGGGGTCAGGGATCATGGGGGAGAGCGCTCTGGAGTCG
GGGCCTGCGCCCGGAGCGCCGGCAGGGGGTCCGGTGCACGCCGTCACGGTGGCGACCCTGCTGGAGAAGCTGGCGACCATGCTGGAGAGGCTGCGCGAGCGGC
AGGGGGGCCTGACTCAGATGCAGGGCGGGCTGGCGGGCTCCGTGCGCCGCATCGAGAGCAACCTGGGCGCGCTGAGACGCAGCCACGATACCACAGTTTTGAC
GCTGGCGCAGCTGCTGGCCAAGGCGGAGCGCGTGGGCTCGCACGCGGATGCCGACCAGAACGCGCCGTGCGCCGCGCGCCCAGAGCAGAGCTGGAGACACCAC
GACTGTGGTGCGCGCGGTAGTCCACGTCTGCCTTAAGAGAAGCTGAATCCCACCAGCCTTCANAAGCGCGGACCCTAGCCCGGGAACTGGCCAACTGCCGACC
GACGAAAAGTCAACAGAGAGCCGGGAGCAGCCGAGCGAGGACCGGTGAGAAGACAACTGCAGCCCTCAGCCAAGGCGCGCCACACAGCGTAGCCCCGCTGGCG
CGAGNTGGCCGAGACACCTGGGCACGACAACCCCAACGGGACGGACGGGCGAG
>‘000128a-052.scf’ came from CONTIG 43 at offset 0;
“E:\SEQUENCE\export\EST_db\000128a\000128a-052.scf” (44>569)
AATTGGCACGAGCTCAGGGCACCAACGACTCACTGATGAGGCAGATGAGGGAGCTAGAGGACCGCTTTGCTAGTGAGGCCAGCGGCTACCAGGACAACATTGC
CCGCCTGGAGGAGGAGATCCGACACCTCAAGGATGAGATGGCCCGCCACCTGCGCGAGTACCAGGACCTGCTCAATGTGAAGATGGCGCTGGCACGTGCAGAT
TGCCACCTACCGGAAGCTGCTGGAGGGCGAGGAGAGCCGGATCAACCTNNNNNATCAGACCTTTCTCTGCCCTCAACTTCGAGAAACAAGCCNCGACAGAGGG
GGTGTGAAGTCATACCAGAAGACGNGATGATCAAGACATGAGACCGGNAGGNNGAGTCGGAGTGAGGCAACACAGAGATGAGTGCTCTAAGCAGAGTTTTTGC
TGCAANACGGCTCACTTGTCTACTGCTCTAAGCANNCTCTCTTCAGCACACCCACCATGTTCCTCAACTTGACTGTTCGGACCCTTTGTCCCAGGAAAGACAC
TCAGCAGTACCC
>‘000128a-053.scf’ came from CONTIG 44 at offset 0;
“E:\SEQUENCE\export\EST_db\000128a\000128a-053.scf” (43>620)
ATTTCGCACGAGGGGAAGGTTTTCCTGGCAGGTTGATTTCGAGTCAGCATGTCGGCTTCAATGAAGGAATGCCTTCAGCTTCAGCTGCTGGAGATGGAAATGC
TGTTTTCTATGTTTCCTAACCAAGGAGAAGTAAAACTTGAAGATGTCAATGCCCTGACGAACATAAAGAGATACTTGGACGGCATAAGGGAGGCCTTGCCACC
AAAAATCGAATTTGTGATCACCCTGCAGATCGAGGAGCCCAAGGTGAAAATTTTCTTGCAAGTAACCATGCCTCACAGGTACCCCTATGTAGCACTACAGATG
TGTGCACGGTCTGCAGAACTTGACAGACAGCAGCAGCTGCTTCTCAACAAAGGCCTCACTTCCTACATCGGGACTGTTTGATCCCAGGGAGCTCTGTGTGTGG
GCGGCATNCAGAGTTACAGACAACAGGCCTCCTACTTCTGACAGAAGCTGTGGACGAACATGAACAGCAAGCCATCAGAACACTCTNCGATGTGACTACGCAC
ATATJTCAGAGACCTCGAAAGACTGAGNCGGAAGTNGACGACGAATTGAGAAGAAGCGGATACG
>‘000128a-054.scf’ came from CONTIG 44 at offset 0;
“E:\SEQUENCE\export\EST_db\000128a\000128a-054.scf” (39>53)
GGTCTGCAGTTGCTC
>‘000128a-055.scf’ came from CONTIG 46 at offset 0;
“E:\SEQUENCE\export\EST_db\000128a\000128a-055.scf” (10>290)
CGTTAACTGGGTCCCCGGGCTCAGTLTCCGCCCCCGCCCCACGCGCCCAAAAGGCTGGAGCCTCATCGCTCCATGTGTGGTATTTGGGGCTCTTTGGGGGAGA
TGACTGGCTTTTTGTTTGTGGGTGATGGGTTTTAATATTGCTCACAGGGGTCCGGGAGCATTTCGTTTTGAAAATGTTAATGGGTACACCAATTGCTGGTTTG
GATTTCACCGGTTGGAGAGAAGATGACCAGCTGTGTGGTATGTCTGCCGATCCGAGAGAAAGAAATTATNCTCCTT
>‘000128a-057.scf’ came from CONTIG 47 at offset 0;
“E:\SEQUENCE\export\EST_db\000128a\000128a-057.scf” (53>548)
GCACGAGGCCAGTACACAGGTGTCGTGAAAACCACCGTTAAACCTAAGCCAAAAGGGAAAGGAGAAGACCCACATCAACATCGTTGTCATTGGGCACGTAGAT
TCAGGGAAGTCTACCACGACTGGCCATGTGATCTACAAATGTGGCGGGATCGACAAGAGAACAATTGAAAAGTTCGAGAAGGAGGCTGCCGAGATGGGAAAGG
GCTCCTTCAAATATGCCTGGGTCTVGGACAAACTTAAAGCTGANNTCTTGCGTGGTATCACCATTGATATCTCGCTGTGGAAATTTGAGACCAGCGAGTACTA
TGNTACCATCATTGATGCCCCCAGACACAGAGACTTCATCANAAACATGATTACAGGCACATCCCCAGCTGACTGTGCTGTCCTGGATCGTGTGCTGGTGGTG
GTGGATTTGAAGCCCGGATCTNCAGAAACGGCAGACCCGNGAGCATGCCCTTTTGGCTTTACACCTGGTGNNGAANCACTANTG
>‘000128a-056.scf’ came from CONTIG 47 at offset 20;
“E:\SEQUENCE\export\EST_db\000128a\000128a-056.scf” (77>499)
TGTTGTGGAAACCACCGCTTAACCTAAGGCAAAATGGGAAAGGAGAAGACCCACATTAACATCGGTGTCATTGGGCACGTAGATTCAGGGAAATCTACCACGA
CTGGCCATCTGATCTACAAATGTGGCGGGGATCGACAAGAGAACAATGAAAAGGTCGAGAAGGGAGCTGCCGAGATGGGAAAGGCTCTTTAAATGCTGGTCTG
ACAACTTAAGTTTTTGACGGGGTCACATGATTCTCCTGGGAATTGAACAGAGTATATGTACTATGAGCCAAGAACAAACTATCAAACTGTTCGGCCTCCAGTG
ATGGCTGCGATGGTGTGGTTGGATFGAGCGATTTCAACGCAACCGACTGCCTTGTTACTGGGGAAATATGGGGTACAAGATCCTACCCTANCAAAAACAATGT
GAAACCTTTAA
>‘000128a-058.scf’ came from CONTIG 48 at offset 0;
“E:\SEQUENCE\export\EST_db\000128a\000128a-058.scf” (9>565)
GCGTTAAACTAGGGATCCCCCGGCTGAGTATTCGGCACGAGGGATCCCACAAGAGGCTGGACCCTAGACATACTCTTGAATAACGCACACATGGTGTATAACT
AGCACGCACGTAAAACATCCGTTTTACGTAGCCACAGGAGGGGGAAACCATATAGATGAAAACACTGAGTCTGAGACGTGGACATCCCAGAAGTGTATAAACT
AAACTTACCTGAGTATAGGAAGAACCTCTGATATCCGTGAGCAAGACAAGGAAAAAGAAGAATTCTGAATATATGGGAAAGCCAGCGCTATAGGGAGAGCAAC
AAGGACCATGAGAGCCAGATATATGATGGGGAATAGAGAGGACACACGAGGATAACATACACATAGAGAAAGCTATCAACGGGGAAAAATAAGCGATGAGACA
CTCATGGTGGACTGAACGGCCGGCATAGGGAGGCAACGATAGTATACGAGGGAAACGAACAATATGACGCAATAAACGAGTAGAAAATGGGAGCTACACAGTG
TTTAGGAGGAGCTATGGACAAACGGGTTTTAAATTCCGG
>‘000128a-059.scf’ came from CONTIG 48 at offset 0;
“E:\SEQUENCE\export\EST_db\000128a\000128a-059.scf” (40>518)
GTCTGCTATTCGGCACGAGGGTTTACCAGCTTAGAGGTCTTGGACCAATTGAAGAAGACACTATTCTTGTCATAGATCCAAATAATGCTGCAGTACTCCAGTC
CAGTGGAAAAAATCTGTTTTACTTGCCACATGGCTTGAGTATAGATAAAGATGGAAAGTATTGGGTCACAGACGTGGCGCTTCATGAGGTGGTCAAACTAGAT
GCAAAGAGTAAAGAAGGCCCTCTGCTAACCGTGAGAAGGAGCATGCAACCAGGCAGTTTTCTAGAAACACTTCTGTGCAGCCCACCGATGTGGCTGNGGGACC
AGACACCGGAACCATCTATGTGTCAGATGACTACTGCAACAGTCGCCTTGTGCAGAGTTCACCAAGTGAAAAATTCATCACACAGTGGGGAGAAGCGTCTTAG
AAAACAANCCTATACAGACCCAGTCAGAGTTCTCACAACTTGCCCTGTNGCCTCCCTGGCCACTGGG
>‘000128a-060.scf’ came from CONTIG 50 at offset 0;
“E:\SEQUENCE\export\EST_db\000128a\000128a-060.scf” (45>469)
TTCCAATGAATATTCAGGACTGATTTCCTTTAGGATTGACTGGTTGGATCTCCTTGCAGTCTAGGAGGTATTACTTACATTGTAAAATTTACCCCTGTAAAGT
ATACAATTCAGTGGGTCTTTATATACTCACAGTTATATTTAATAACACTAATCTCAGAAATTTCATCACCCCCAAAAGAAGTCACATACAGATTTAGCAGTCA
CTTCCCATTTTCTTCCTNCCATCTCCAAGCAGCCACTATACTAGTNNNNACTATGATTTGTCTACTTTGGACTTTAACTATATGAATTCTACAATATGGGCTT
TTGTGACCATTNNCTTTACTTTACAAATATTGATATTGTGGAGGGCAGGGGCATACTCATTCTCCTCTGTTCTTAGTAGATGGNGAGAGAGAAAGAAAAAAAA
AAAATGTAAA
>‘000128a-063.scf’ came from CONTIG 51 at offset 0;
“E:\SEQUENCE\export\EST_db\000128a\000128a-063.scf” (49>634)
CGCTGACTATTCTCAACCAACCATAAAGATATTGGTACCCTFTTATCTACTATTTGGTGCYTTGGGCCGGTATAGTAGGAACAGCTCTAAGCCTTCTAATTCG
CGCTGAATTAGGCCAACCCGGAACTCTGCTCGGAGACGACCAAATCTACAACGTAGTTGTAACCGCACACGCATTGTAATAATCTTCTTCATAGTAATACCAA
TCATAATTTGGAGGATTCGGTAACTGACTTGTTCCCCTAATATTGGTGCTNNNNTATAGCATTGCCCGAATAAATATATAAGCTCTGACTCTCCCTCCTCATT
CTACTACTCTGCATCTCTATAGTGAGCTGGGCAGGACAGGTGACCCGGTACCTCCTTACAGCAACTAACCTGCAGAGCTAGAAGACTAACATTTCTCTTAACT
AAAGAGATCTCATTTAGAGCACACTTATACACATATAAAAAGACCCGCAGGACATACAACCTTGTGTGAACGATATACGGGACATATACTTCCTGTTTAAGGC
TACAAGTTTAAACGAACAAAACCTTTGACGGGAGAGGACTTTTTAACTTTTGTTTGCCCGAGTTTTATTTCG
>‘000128a-064.scf’ came from CONTIG 52 at offset 0;
“E:\SEQUENCE\export\EST_db\000128a\000128a-064.scf” (54>595)
GCACGAGGGGGATCTACACTCGAGTCAAGAACTACGTTGACTGGATAAGGAAGACAATGCAGGAGTATAGTGCCCCCAGTGTAGACTAACCATACAGGTCCCA
CCAGCCTCTCTAAGGGCTGTGACCGCTCTGGACTTTCTCTTCCTCACAATAGTTCCATTATTTGACCATGACTGAGAGAGGACACGGGAGTGAGATTGAGCTA
GTGCCAGGACTTGGATGTCGGGACACTGGGTTGGAGGTAGGGTGTNTNTCCTGTGGCTGTGTTGGTCTTTCAGTATAGATGGACTAACTACATGGGGTCCTCT
CCCCCGAGTCCATCCTGTGGAGTTCAGTGTGCAAGGGAAACCCTCTCTTTCTCTATTCATGGGTGGNNAGGGTCCTCTTCTGGATGACCCACTCCTGTTACAG
ATCTGACTCTGAAATTTGCTGTGGGGCATTCTCTTGATTTTGGGTTNCCCTTTACCGTTGAAGTTGACCACACGTTCTGCTACTACTGTAATAAGCATGTTAT
AACCCAAAAAANAAAAAACTGGA
>‘000128a-065.scf’ came from CONTIG 53 at offset 0;
“E:\SEQUENCE\export\EST_db\000128a\000128a-065.scf” (50>578)
GCACGAGGTGAAACCTTAAATCCCACAACATTTATTATAAAGGTGCTGTAAAGGGAAACGCTGGGCTTCATGACGGGCTTATCGGTAGGATTTCTGGTAGCGG
GCACGGGCACCAGGACCTCCAAACTTCTTGGATTCGCAGCGACGGGGATCGGCTACCAGCAGGGTCCGGTCATACTGGATGAGGATGTCTTTGATCTCCTTCT
TGGAAGCCTCATCCACATATTTGTGGTAATAGGCCACCAAGGCTNATTAGATGGACTGGCGGATGGCGTAAATCTGGGCGACGAGACCACCACCCTTCACTCG
GACGCGGATGTCCACACCAGCAAATCGCTCCTTGCCCNAGAGCAGAACAGGTNCCAGTAGCTTGTATGCAGCGTGCGCGGTTCGATCATTCTCAGGGTCGTGC
GTTCACCTGATGAGGNCGNTACCTCGTTTGCAGTGCGCCAGGCTGTGGCCGNCTTCTTTACGNCCGAGACTTGCACGACTGCAGAGGCCCTTTGGACGCATGG
CTCAGGCGCAGAACG
>‘000128a-066.scf’ came from CONTIG 54 at offset 0;
“E:\SEQUENCE\export\EST_db\000128a\000128a-066.scf” (50>515)
CCGGGGACGAGGGCCTGACCCACTNACAAACGTGTGCTAGACTACCCGAGGGACCCTTGAAAGCCAAAGCTCGGTCCCCGGTAGCGTCGAAGGCTACGAATCT
TGTCCGCACCAACGCCGACCTGAGGGGGGAGGGGCTGCAGCCAGGAAGACAGAGCGGAAAGAACAAAGGAGGGCGAGGAAAACAGGCGAAGCACAAAGAAAAA
AACAATAAACCAGCGCAGTCGGAGGAGGCACACGTGTGGATGGGATGAGCTCTTCTATGAGAAGGACAGCGCCGGTGCAGCCAGACCTGAATGCGAGGAGGAG
GAAGAGACAGAAGTGGGAGAGGAGCAGGAGTAGGCGGCAGATTGGACCTAGCACAGCTACAGAGAATGATGGCTGGGGGGAGAAGAGGCTGGATAGAGCGCGG
GTAGATATGACACAAAAAGATGACGAGAACGCGCTGAGAACACGGGAGCAGAGA
>‘000128a-067.scf’ came from CONTIG 55 at offset 0;
“E:\SEQUENCE\export\EST_db\000128a\000128a-067.scf” (9>545)
CCGTTAAACTAGGGATCCCCGGGCTGCAGGCAGAGACGGGCGCCGTCCCATCTCGGCCTCTGGGTAACTTCTGCTTGACTGACCGACCGTGACCCTGACATAG
CGTCATATITCATGGCAGCCAAGGGAGGCACTGTCAAAGCCGCTTCAGGCTTCAACGCTGCCGAAGATGCCCAGACCCTGAGGAAGGCCATGAAAGGGCTTGG
CACAGATGAAGATGCCATCATCAACGTCCTGACCTATCGCAGCACGGCCCAGCGCCAGGAAATCCGGACAGCCTACAGTNTTTCCATCGGCAGGGACCTGATG
GACGACTTGAAGTCAGAACTGAGTGCAAACTTCGAGCAGATGATCCTGGAGATGATGACACCCCACGAGCTGTACGATGTGCAGGAGCTGCGTAAGGNCATGA
AGGNAGCTGGCACAGATGAAGGCTGCTGATTGAGATCCTGGCTCCNGACACCGAGAAGAACGGCGCATAACAGACCTACACTGCATATGCGNAGCTGAAAAGA
ATGGTGGACCGCATCATGTCACG
>‘000128a-068.scf’ came from CONTIG 56 at offset 0;
“E:\SEQUENCE\export\EST_db\000128a\000128a-068.scf” (51>578)
CCGGGGCTATATTTATfGGCGCCTTCTCTCAACTGATCCTGTCACAGCTAAAGAAGTAGTCTTGTCTGAAAAGCCACTGATTTCTGAGGAGACAGATCTTATT
GAGCCAACTCTACTGGATGAGCTAATCTGCCACATTGGTCTTTGGCCTCAGTGTACCATAAGCCGCCCAATGCTTTTGTGGAAGGAAGTCATGGAATCCATCG
CAAACAGTTGCCAATACATCATGGGAGCACTGATGCAGGAGACAGCCTTNTNGGGACCACCACTGGCACCAACCTGAAGCAGCCTCAGGTTATTCCCTCCAGG
TGACCTTTGNGGGATCTTTAAACCTGACCNNTGTCCCAGGCATGTGCACAGTATCTLTCATGCAATGGAGCAGGGATCTCTGGAGANGCTAAAGGGGNGGGAA
GCCTCTCCACATCAGGCGCACCTGTCCTCCTATCTGCGGCAGAAGGCGAGAACGTGACTTCCGGAAGCAGCACGGGAAGGGTCAGTGTGCGCTGAAAGTAAAC
GGAATAAAATATA
>‘000128a-069.scf’ came from CONTIG 57 at offset 0;
“E:\SEQUENCE\export\EST_db\000128a\000128a-069.scf” (49>593)
TCAGGACAGAAATGTACAAGCTCATGGTCCGTATTCATAAATAGGAGATTTCGGGAAGATGAGACTGGATTATAAACAGGATCAGTCAGAAGCTGAAGATCTT
AAGGATATGCAGTGGAAGGCAAACATCTCTTCATTCCACAACAGAAAACTATGACGATCAGAAACTTTAAGTGAAAAAAAAAAAACAACGTTGTAAGAGGAAC
ACATCCTTCAGATAACCAAACAATGTAAAATACTGCAGGGTTTTGACCGTTNTGTGGAGTGTAAGAAGGTGGACTCAATTTCAGTGTCACTCTAATTCGCATT
GTGGATCAGAATCTTGGAGCCAAAAAAAAGGAAATCCAATCATAGCACANAGCTTGGTGGNTTATTGAATAACATTTANATAATCATAATGGAAANTGCTGTP
ATGGNNTTCATCTTTTTTAGAGAATGCTATTACGGTACAGANTGNAGTGNCATATTATCACCTGATGCGNANNNAGGCATACAGGNAGATGCAGGNCGGCGCT
GGNAAGNAGGNNGNCTTTAAGNGGNAGCGN
>‘000128a-070.scf’ came from CONTIG 58 at offset 0;
“E:\SEQUENCE\export\EST_db\000128a\000128a-070.scf” (50>628)
CGTGGCTACAGCTTCACCACCACGGCCGAGCGGGAAATCGTCCGTGACATCAAGGAGAAGCTCTGCTACGTGGGCCTGGACTTCGAGCAGGAGATGGCCACCG
CGGCCTCCAGCTCCTCCCTGGAGAAGAGCTACGAGCTTCCTGACGGGCAGGTCATCACCATCGGCAATGAGCGGTTCCGCTGCCCTGAGGCTCTCTTCCAGCC
TTCCTTCCTGGGCATGGAATCCTGCGGCATTCACGAAACTACCTTCANTTTTTCATGAAGTGTGACGTCGACATCCGCAAGGACCTCTACGCCAACACGGAGC
TGTCCGGCGGGACCACCATGTACCCCGGCATCGCGGACAGGATGCAGAAAGAGATCACTGCCCTGGCACCCAGCACATGAAGATCAAGATCATCGCGCCCCCT
GAGCGCAGTACTNCGTGTGGATGGNCGCTCCATCCTGGCTCGCTGCCACCTTCACAGAGNGGATCACAGCAGAGACGAGAGCCGCCCTCCTCGGCACGCAAGC
TCTAGCGACGTAGCGCGTACCCTTTCTGCAAACTACTGCCAAACGAAGAAATGAGTCTTTGTTT
>‘000128a-071.scf’ came from CONTIG 59 at offset 0;
“E:\SEQUENCE\export\EST_db\000128a\000128a-071.scf” (55>577)
GCACGAGGCAGGAATGGGGCCCTTACCAAGGAAAAGTCGGTGAAGCATATGGACTTGTGCCTGACCGTGGTGGACCGGGCACCTGGCTCCCTCATAAAGCTGC
AGGGCTGCCGGGAGAACGACAGCAGGCAGAAATGGGAGCAGATCGAGGGCAATTCCAAGCTGCGGCATGTGGGCAGCAACCTGTGCCTGGACAGCCGCGCAGC
CAAGACGGGCGGCCTGAGCGTGGAGGTGTGCGGCCCGGCGCTTNNTTAGCAGTGGAAGTTCTCGCTCAACCTGCAGCCGTAGGGGAGCCTCCCGCGTCTGCCC
GCGCCCGGCCACCCAGCGACGAGCACGTCATCAAGTCTGTTTCTTAATACTTNCGAGAAACTATATACCTCAGTATTCATCATGTCTGCAGGTCGGAGACTAG
CGCGGNGAGGGCGCACCAGAGCGGAGGAGAGGAGCTNTGCGCCCTCTCGCCTGCGCTGGCGNCCACACCCTGGAGCACGGNCCGAGNNNGACGGAAGAAGGGC
CTGCCAGG
>‘000128a-072.scf’ came from CONTIG 60 at offset 0;
“E:\SEQUENCE\export\EST_db\000128a\000128a-072.scf” (36>612)
GTGCTGCATGAATTCGGCTCGAGGCTGCCCGGTGCTTGCCTGCTGCCTGCCTGCCTGCCACTGTGGGTFCCCAGCACCATGAGGGCCTGGATCTTCTTTCTCC
TTTGCCTGGCCGGGAGGGCCTTGGCAGCCCCTCAACAGGAAGCCTTGCCTGATGAGACAGAAGTGGAGGAAGAAACCGAGGCCGAGGTGGCCGAGGTACCCGT
GGGAGCCAACCCCGACCAGGTGGAAGCAGGAGAATTCGATGATGGTGCCGAGGAAACCTTCTAGGAGGTGGAGGCCGAGAACCCCTGCCAGAACCACCACTGC
AAACACGGCAAGGAGAGAGAACTGGACGAGAACAACACCCGCTGTGTGAGAGCCAGCACCCCACCAGCTGCCCTGCCCGCATCGGCGAGTTGAGAAAGAGTGC
AGCAACGACAAACAGACCTTCGACTCTTGCTGCCCACTTCTTGCAGCAGNGGACACTGGAGGCACCAGAAGGGCACAACTGCACTGACTACACGGGCCCTGCA
AACATGCCCCCTGCTGGACTCGAGCTGACGATTCCCTGCGCAGCGGACTGGCTAGTACGACT
>‘000128a-073.scf’ came from CONTIG 61 at offset 0;
“E:\SEQUENCE\export\EST_db\000128a\000128a-073.scf” (55>610)
GCACGAGGCTTCAAGGCCCTCATTGCCGCTCAGTACAGCGGGGCTCAGGTCCGCGTGCTCTCCGCACCACCCCACTTCCATTTTGGCCAAACCAACCGCACCC
CCGAATTTCTCCGTATATTCCTGCTGGCAAGGTTCCAGCCTTTGAGGGTGACGATGGATTCTGTGTGTTCGAGAGCAATGCCATTGCCTACTATGTGAGCAAC
GAGGAGTTGCGGAGAAGCACTCCCGAGGCAGCAGCACAGGTNNNNTCAGAGGGTGAGCTATGCTGATAGCGACATAGTGCCACCGGCCAGCGGGGGGGGTGTT
GCCTACCTTTAGCATCATGCACCACAACAAAGCAGCCACAGAAGATGCAGAGCAGGAGGTGAGGCGAANTCTGNNGCTGCTGGATGGTCACTTGAAGACGAGA
CTTNTCTGGGTGGCGAACGCGTGNACGCTGCTGAATCACAGATGTCTGCACCTGTGTITGGTTTACAACAGNTCTGGAGCCTCTLTTCGCCAGNCCTTCCTAT
ACCCACGCTGGTCTTACTGCATATCANCCCCATCGGNCTGTTG
>‘000128a-074.scf’ came from CONTIG 62 at offset 0;
“E:\SEQUENCE\export\EST_db\000128a\000128a-074.scf” (16>50)
AATGACCCCGGCTGATAATCGTIGAGGGGAGCTGA
>‘000128a-075.scf’ came from CONTIG 63 at offset 0;
“E:\SEQUENCE\export\EST_db\000128a\000128a-075.scf” (56>567)
GCACGAGGGGATTTTGCAGACATGATTCATATTTCCTTCTTAAAGTGTATTTCTTTCTGCTCGCAGTTAAACAGAGAAGGAGGGTCTGGCGTGGCCTGTCTGA
GATTGTTAATGATGTAAATTGAGTCCCTGGTTTITTTACTTCCGTCTCGTGTCACATGACCGGCGTGCGCATGGAGTAGAAGGATGATGCTGAGAAGTCAAGG
AACTGAGCACGCGGACGAACAGAGGCCGCGTCAGGCGCCTTTCCACCAACCCACCCTCTCCCCTCAGTTTGGTTGTTAGTCTCACCCAGTCTCCTTGAGAAGA
TGGAGGGAGGCTGACACAACAGCGCGACACTACCCTGTGCCCCGCCCGACCATCACGAGCCTACGTCCTCAAGAGCGGTGCGGCGCTGTGCGTGGAGAGTCAA
ACAGTATATGTGTATGAAACATGTACACATCAAGTTATGATATAAGATCTCAACTTCTAATTTAATTTTAAAACTGATGTLTGTCTAGGGGGGTTTGTCTT
>‘000128a-076.scf’ came from CONTIG 64 at offset 0;
“E:\SEQUENCE\export\EST_db\000128a\000128a-076.scf” (50>486)
CTCGTGTGGACTTTATCTTTGAGGGCAGAGAAGAAAGTAATTTTAAGTGTGGAAGATAGATTGATTATGTTGGAATTCTGACACCGTAGCTCTATGGTACATG
AAGCTCCCTCATATGAACCTTCAAGTTGAGAGCTTTGTAAGATGCTGATGTGTGTTCACGTGTCCGGTCACATAAGTTAGTTGACGTGTCTGGCGTACTTCTC
AAGGTAGTGTACTGTAAGATTAAAAGATGTTGTATTTTTTTGTGTTTGTNNTAAGGTATTATTTGGTGAAAAGGATTGGGAATCCTACTACACTACAATACTA
TACAGTTGATTGTTAGATGGGTACCTAGGCTAACTNNTGTGGACTTATGAACAAATTGGATTTATGAACATGCTCTTAAATGGAACTCATTCATATGAAGGNG
ACTTACTGTTATAGTAGATGGGGAGG
>‘000128a-077.scf’ came from CONTIG 65 at offset 0;
“E:\SEQUENCE\export\EST_db\000128a\000128a-077.scf” (47>555)
TGTTGAGTGGTTGTGAGATTTAAAAAGCAAAACAGACCACTGCTGGCTGGTTGGTGCCGAGGTGAATCACCCAAAAGCTCCCATGCCACAGAGCGAAGCCGAG
ACCACGAAACCTGGGTTCGAGGCCCAGTGGGAAGGGGTGCATGGCCTCACTCCTCACTGCTGCTCACTTCCTCCAGGGCAGCATCTCCCCTGNGTTTGCACCC
ATGGGACTCATCTTTTGGGAGGGTTTTTGTGGTTGTNTTGTTGTACCTTTTTTTAAGGAGCAGAGAGGCCAGTGATCACCCCCGAGCCGGGCTGGGTAGCAGG
TGACCTGACATGCGNNGATGTGCCCTCAAGAGCCTGGNGCCTTCACCGCTTTGTGCTTGGGCTGNGTGTGCCTCGCTCCCTCTGGGGCGGNGGCCGCTGCAGA
CCTGCCCNAGGGTGCAGNNCGGGAGCCACCACTGATCATACCAGGCTGNCAGCCACGGACGCTCGCCTGGTCTGCTCACTTCCCTGGCTGGANNAGT
>‘000128a-078.scf’ came from CONTIG 66 at offset 0;
“E:\SEQUENCE\export\EST_db\000128a\000128a-078.scf” (49>560)
TTATTCGGCACGAGGATCATTGATGCCCCAGGACACAGAGACTTCATCAAAAACATGATTACAGGCACATCCCAGGCTGACTGTGCTGTCCTGATCGTTGCTG
CTGGTGTTGGTGAATTTGAAGCCGGTATCTCCAAGAACGGGCAGACCCGTGAGCATGCCCTTTTGGCTTACACCCTGNGTGTGAAACAACTAATTGTTGGCGT
TAACAAAATGGATTCCACTGAGCCACCCTATAGCCAGAAGAGATACGNTTNAATGGTTAGGGAAGTCAGCAGCTATATTAAGAAAATTGGCTACAAACCCGAC
ACAGTAGCATTTGTGCCAATTTCTGGCTGGAATGATNGACACATGCTAGAACCAGTGCTATATGCCATTLTCAGGGATGGAAGTCACCCGTAGGGACGCATGC
CAGGGAACCACCTGCNTGAGCTCTGATGCATCTGCACCACTCGCCACTGACAACCTGGGTGCTCTCAGAGCTATAATGGGGATGTACGCCTGGGNCGGG
>‘000128a-080.scf’ came from CONTIG 67 at offset 0;
“E:\SEQUENCE\export\EST_db\000128a\000128a-080.scf” (46>302)
TGTTATTCGGCACGAGGTAAGTGTAATCGGCAGAAACAACAGCAACATCTTTGACCTGAACGGGAATTTCCCGGACCAGTTCGTTCAGATCACAGAGCCCACC
CAACCAGAAACTATGGCTGAGATGAGCTGGATGAAGACCTATCCATTTGTGCTGGCAGCAAACCTGGATGGAGGGACTTTGGAGGGTAACTACCCTTTGTGAG
ATGATGAACAAGGCAGTGCCACATATAGGAAATGACCAGATGATGCTGTTCT
>‘000128a-081.scf’ came from CONTIG 68 at offset 0;
“E:\SEQUENCE\export\EST_db\000128a\000128a-081.scf” (51>540)
GGAGATGAACCGATAGATTTCAAAATCAACACCGATGAGATTATGACCTCAGTCAAATCAGTCAATGGACAAATAGAAAGCCTCATTAGTCCTGATGGTTCCC
GTAAAAACCCTGCACGGAACTGCAGGGACCTGAAATTCTGCCATCCTGAACTCCAGAGTGGAGAATATTGNGTTGATCCTAACCAAGGITGCAAATTGGATGC
TATTAAAGTCTACTGTAACATGGAAACTGNGGAAACGTGGATAAGNNTNAGTCCTTTGACTATCCCACAGAAGAACTGGTGGACAGATTCTGGTGCTGAGAAG
AACATGTTTTGGTTTGGAGAAATGATGAGGTGGNTTTCAGTTNAGCTATGGGCATCTGAACTTCCGAAGACGTCTCGATGTCAGGTGGCATCGTNCGACTTTC
TNCAGCCGGCTCTCAAACATCAATATCACTGCAGAATACATGCTACTGGATCATGCAGGGNAATGTAAGAAGCTGAAGT
>‘000128a-092.scf’ came from CONTIG 68 at offset 441;
“E:\SEQUENCE\export\EST_db\000128a\000128a-092.scf” (63>428)
GCTTATAAAGCCATAAACATAGGATACAAGAAGCTGAAGTTGCGGCGGGTAGGTAAGAAAAAATGAAAGGAGAAGAACAGGACGGACACGGGCAGGAGGAAAG
GACCAGGGGGAAGGGCGGGAGAGGGGGACAGAGGAAGGAGGGCGGAGGGGGGGGGGCCTGGAGGAGAGGAGGAGGGAGAGAGGGAGAGAAAAAGAGAGACGAA
GAGAAAAACAGAGGAAGGCAGAGAGGAGACAGAGAGGGACGCGAAGATAGGAGAAGCAAAGGCTAAGTGGCTAGAAGAATGGAAGCAAACAAAGAGACACACG
ACAAGAAAACCACACCGCACGCTACTAAAAAGAATGGAAAAAAAGAAGACAAATITGT
>‘000128a-082.scf’ came from CONTIG 69 at offset 0;
“E:\SEQUENCE\export\EST_db\000128a\000128a-082.scf” (53>345)
TCGGACCCGTGTCGGAACCTGACCGGACAAGAAGACGGAGCATCGGTGAGATGTGTACACGGCGTCACTGGCATGGGTGCCGTGGCATGTGTCACCATATGAG
CACCACGCCTGGATGGCACCGCTGGGCACCGCGGCTTGGCACACCACAAGGGCCCTCGCCGCTGAGATGGACGANAGGAGGTGGAGTAGCAGAGACGTATACA
GCAGGGCGGAACAAGCAGGGACAGTATGATAGGGAGTACATCACTGTGGTGACATTGCATCATGGGATACTCATCATGATGCTGCCA
>‘000128a-083.scf’ came from CONTIG 70 at offset 0;
“E:\SEQUENCE\export\EST_db\000128a\000128a-083.scf” (51>541)
CGGGCGGCCGCCTGGCCCGGGCAGTGACGCGGGCGCTGGCGCTGGCCCTGGTGCTGGCCCTGGTGGTCGGACTGTTCCTGAGCGGCCTGACCGGCGCGATCCC
GACCCCGAGGGGCCAACGGGGACGGGGGATGCCGGTTCCGCCCGCCTACCGCTGTCGCTCGCTGATCCTGGACCCCGAGACGGGCCAGCTGCGCGTGGAGGAT
GGGCGCCACCCTGACGCCGGAGCCTGAGGCCAACCTTACGAACGTTCCACGCGAGAGCGGGAGGGCCTTTGTGGAGCTGCACACGAACGGGCGCTTGAATGAC
AGACTGCAGGCCTACGCCGCAGGCGAGAAGGAGGCTGCTGAGTGCGGAGAGCTGATCTACATGTACTGGATGAACACGATGGAGAATTAGTGCGGACCCTTCG
AGTATGAAGGGGTATACTGTGAGATGCTCAAGAACTTGCTAGAGGGCAACCTGTAGTGGATGCAGAAAGATATGGAGCT
>‘000128a-084.scf’ came from CONTIG 71 at offset 0;
“E:\SEQUENCE\export\EST_db\000128a\000128a-084.scf” (47>645)
CTGGTCCCCCAGGTCCTCCCAGCGGCGGCTACGACTTGAGCTTCCTGCCCCAGCCACCTCAAGAGAAGGCTCACGATGGTGGCCGCTACTACCGGGCTGATGA
TGCCAATGTGGTCCGTGACGGTGACCTCGAGGTGGACACCACCCTCAAGAGCCTGTGCCAGCAGATCGAGAACATCCGGAGCCCTGAAGGCAGCCGCAAGAAC
CCCGCCCGCACCTGCCGTGACCTCAAGATGTGCCACTCTGAGTGNTNTAGCGGAGAATACTGGATTGAGCCCAACCAAGGCTGCAACCTGGATGCCATTAAGG
TCTTCTGCAACATGGAAACCGGAGAGACCTGTGTATACCCCACTCAGCCCAGCGTGACCCAGAAGAACTGGTATATCAGCAAGAACCCCAGGAATAGAGCACG
TCTGGTACGCGAGAGCATGACCGGCGAITNCATTCGAGTATGGCGGCAGGGTNCGATCTGNCGATGGGNCATCCACTGACTTTCTGCGCCTGAGTNCACGAGG
CCNCCANAACATCACTACATGCAGAACAGNGGNCTAACTGACGACAATGGCACTCAGAAGCCTGTNCTCAGGCTCANGGATAGA
>‘000128a-085.scf’ came from CONTIG 72 at offset 0;
“E:\SEQUENCE\export\EST_db\000128a\000128a-085.scf” (304>310)
CAAGCAG
>‘000128a-087.scf’ came from CONTIG 73 at offset 0;
“E:\SEQUENCE\export\EST_db\000128a\000128a-087.scf” (293>298)
AAACAC
>‘000128a-088.scf’ came from CONTIG 74 at offset 0;
“E:\SEQUENCE\export\EST_db\000128a\000128a-088.scf” (302>308)
CAANGN
>‘000128a-090.scf’ came from CONTIG 75 at offset 0;
“E:\SEQUENCE\export\EST_db\000128a\000128a-090.scf” (56>598)
GCACGAGGTCTCCACGTACGGTCAGTTCTACGGTGGTGACAGCTAGATCATTCTGTACAACTACCGCCACGGCGGCCGTCAGGGACAGATCATCTACAACTGG
CAGGGCGCCCAGTCCACCCAGGATGAGGACGCTGCCTCGGCCATCCTGACCGCTCAGCTGGACGAGGGAGGCTGGGAGGGGACTCCCGAGCAGAGCCGAGAGG
GCCAAGGCAAGGAGCCCGGTCACCTCATGAGCCTGAGTTCTCGGAAACCCATGATCATGTACAGGGGCGGGACCTGCGGCGAGGGAGGGCAGACGGCGCCGCG
CCAGCACCGGCCTGGTCCAGGGCCGGGCCAGCAGCTCTGGAGCCACCCGAGCCGTGGAGGTGATGGCCAAGGCTGCGCGCTGATTCCAACGATGCCTTTGACC
TGAGACCCCTCGGCGCCTACTGTGGTGGGTGCGGAGCAGCGAGCAGAAAGATGTGCCTGGACTGTCAGTGGCTCGGCCAACCGGCGGTGCAGAGGCAGAGCCA
AGCTTTGGAGCCTGTGGGAGGCCCTCCG
>‘000128a-091.scf’ came from CONTIG 76 at offset 0;
“E:\SEQUENCE\export\EST_db\000128a\000128a-091.scf” (80>368)
CATTACATATTGTGACTGCTACTGTTATAATGATCCACGATCACTACGAGCACGAATGTTACGACGCTGTTACACGATGAAAGTGGGTGGGCAGTGGCATGGA
TAATGGCGTCACACGAAGTAATAATAATAGCAGAAACTGGCATAAAAGAAATCAATGGCACACACACAACGGCGGAAGGACAACTGCCAAGACAGAACCGCAA
ATGTAGCTACATTGGCTGCTGTAGTGTGGAAGGAGGAGGGAGCGAGGAAAGGGAGAGGGCGAGGCGTGGATGGACGAGTGGGG
>‘000128a-093.scf’ came from CONTIG 77 at offset 0;
“E:\SEQUENCE\export\EST_db\000128a\000128a-093.scf” (52>500)
TTTCGYPTAGAGTTTGTTTATGCTTTATATGCAACTCTCTTATGATTGCACAAATCCTAACTAAAATTCTAGATAGATAAAGCTTCGTGGGGCTAGGTAAGAA
ATTATGCCAAGCTGGAGAACAGCCAGGCCCGGGAGAAAGGAACGACCAGCCGTTAGGCCGGAAGGAGACAGAGGACGAGGCCGAAGGGGGACAAGCTAGGAAG
ACCAGAAGAAAGACAGACAGGGACAAAAAAAAGAAAGCCAACAAAAAAACACATGAAAAAAGGCAGGTGACAAAGAACGACGCGATTATTTGAGAATCAAAGG
CTACTATTGTTTTTAACACTAAAAGTAAAAAACGACGTAGCGGCGATAAGAAAACACCCGGCAAGCTTCTAAAAAAAGAGTGGGAAGAAAAAAGCAAAGTGCA
AGAAACGGAAATCAAAAGCTAAATGAAAGGCCACTCC
>‘000128a-094.scf’ came from CONTIG 78 at offset 0;
“E:\SEQUENCE\export\EST_db\000128a\000128a-094.scf” (1>358)
CTCAGGGGCGGCGTCTAAACTAGGTCCCGGCGCAGCCTCTGGCTGGTTCCCTACACGACCTTCCCCTAGCATCTCCAGACCATCCATTGAGCGTCGTACATGA
CGACACTAAGGACACCTTGAAGCAACAGGAAAGAGAACAGAGGGGGAAAGAAGAGAGGGAACCAATAGAAGTGGGGAAAGGAGGAGGAAGGACCAAGGGAACG
GCAGGACAAAGGACGAAGTTCTAGAAAGGAAAACGGAAAAAGAGAAGAAAAGGAGACCAAGAAAAAAGAAGAAAGGCAATAAACAAGTAAAAGAACCACGATG
GGGAGGAAGGGGAGAAACGCACACATAGCGGCAGGCGGGACCGGGGAAA
>‘000128a-095.scf’ came from CONTIG 79 at offset 0;
“E:\SEQUENCE\export\EST_db\000128a\000128a-095.scf” (53>524)
CGGTCTGGGGCTGGTGTPCCAGCGCCACATACCTTAGCACCAGATGGACAGCTCCACAGCCCAGAGTCTCCTCTGTGGCCTGAGCTACACCTCTGCCGGCTCC
CTCAGCCGCCACCTCTTGATCGGCCACAAGGTGAGAGACCAGGAGGAGGAAGAGGCAGAGGCGGCAGAGCCAGAGGAGGGGCTGGGGGAGGAGGTGCCCATGG
GGACCAAGGAGAACGGACTGGAAGAAAGTGCCGAGGTCTCCAGTGTGAGGCGACTCCGAGACGAAGAGATCTCTGAGCCTGGCCCAGGATGAGGATGGAGCAC
GAGATGCTCCTAAACGACCACAGACCTGTCAGGACCGGGACAGCCACACACCGAGACCCGCAGTGTGACCAGAGGCCCTGCAAGGCATGGGGCGGGTGGACGC
GGGNCGACGCNGCGCTGCTGTGGGGACAAGGGAAANAACAGCCGACCAGGGNGAGTGGCG
>‘000128a-096.scf’ came from CONTIG 80 at offset 0;
“E:\SEQUENCE\export\EST_db\000128a\000128a-096.scf” (42>393)
GGTCTGCATGCGGTTTTTTTTGTGGTTTTTTCTGGCGTCAGACGTTATATGTTAGTGGTGTGGGACTTTACTAGTTCTAGACTGATATACGATCCTGTATGGT
GTGCGGTGTGCTTTAAAATGAACACACAAAAGACATAATGGAGAAGAGGAAAAGAGGAGGAAAGGAGAGAAGAACGAAAAAGAGGGAGCGGAGTGGAGAGGAA
GGGAGGGGGAGGAAGAAAGAAAAAGGGGAGGGAAGAGAGAAGGGGAGGTCTGGGGAGGAGAAAGGGAGAAGGAGGAAGGGAGAGAGGAGAAGAGAAGAGTGAG
ATAAAAAGGACAGACAAGAGAACAGAAGAGAACAGAAGGAGAGA
>‘000203a-001.scf’ came from CONTIG 1 at offset 0;
“E:\SEQUENCE\export\EST_db\000203a\000203a-001.scf” (45>465)
GCACGAGGCTGTTTTATATTCGCCCATTCCAGTCCATTTTTAATTCTCTGATTTCCTAATATGTTGATGITCACTCTTGCCATCTCCTGTTTGACCACTTTCA
ATTTGCCTTGATTCATGGACCTAACATTCCAGGTTCCTGTGCAATATTGCTCTTTTTATCATCAAACTTTACTTCTATCACTAATTACATCCATAACTGGGTG
GTGTITTTTGCTTGTTTTCATTCTCTTCTTCTTTTTGGAGTATTTTTCCACTGATCTTCATTAACATATGGGGCACCTACCGACCTGGGGGGGTGATCTTTTC
ATGTCTTTCTTTTTGCTTTTTCTGTTATGGGGTTTCAAGCAAGATATGAAGAGTTTGCTTTCCTTTTCCGGGACACGTTTGTCAGATCACACAGACTGCCGCT
GGGGTTG
>‘000203a-002.scf’ came from CONTIG 2 at offset 0;
“E:\SEQUENCE\export\EST_db\000203a\000203a-002.scf” (48>297)
GCACGAGGGATTCTTATACTTTCTGAGGGAGTTTAATGACCACTAGAGCTTGTCCTCATATTTTTTTCAGCTTAATACTGTATGTCTCGTAAGATGGGCCTTA
TTGCCTGTATTCTITGATATGTGATTAAGCCTATAGCTTTCAGTGACCAAACATTTTACAGAGTAAAAAATGTTAGGAAGCAGAAAAAGAAAATCTGATTTAT
TCTATGTCTCATTTATCCAGCCCTGCACTTAGATAGAAGTGTGC
>‘000203a-003.scf’ came from CONTIG 3 at offset 0;
“E:\SEQUENCE\export\EST_db\000203a\000203a-003.scf” (40>551)
TTTTGACAGAGAACATTTTTCTCACATACACTTTCAGAGTCAAAGCTGTGGATGGGGGAGATCCCCCCAGATCTGCAACAGCCACGGTCTCTCTCTTTGTGAT
GGATGAGAATGACAATGCTCCCACTGTCACCCTTCCCAGAAATATTTCCTACACTTTACTGCCACCTTCGAGTAACGTCAGGACAGTAGTAGCTACGGGGTTG
GCAACAGACAGTGATGATGGCATCAATGCAGACCTTAACTACAGCNATGGGGGAGGGAATTCCTTCAAGCTGTTTGAGATTGATTCACCAGTGGGNGGGTTTT
CTTAAAGGAAACTCACCCAAAGCATTATGGCTTGCACAGGNTGGTGTGCCAGNGATGACAGGGGCAGCTTCCCATCTACACGATCTGTGCTGTGTTGTCATGA
AAGGTTCTAAGCACTGGATGACTCCCAAAGCAAAACTGCCNCCCCATCACCAGAATATACGGGACCAGCTATAATTACACAAAATAATTTGGGGGGGG
>‘000203a-004.scf’ came from CONTIG 4 at offset 0;
“E:\SEQUENCE\export\EST_db\000203a\000203a-004.scf” (43>365)
GCACGAGGGCCCTTTGACGTTCCGGCCGCGCGCCCCGCGCCTCGTCGCTATGCCTCGCAATTGAGGAAATCAAGGACTTTCTGCTCACAGCCCGCCGCAAGGA
CGCCAAGTCCGTCAAGATCAAGAAAAATAAGGATAATGTGAAGYTAAGTTTCGATGCAGCAGATACCTTTACACCTTGGTCATCACAGACAAAGAGAAGGCAG
AGAAGCTGAAGCAGTCCCTGCCCCCCGGGTTNTCGTGAAGGAGCTGAAAATGAACCACGCATGCTGCTTTGAACTGTATTAAATTTTTTAAACTCAAAAAAAA
AAAAAAAA
>‘000203a-005.scf’ came from CONTIG 5 at offset 0;
“E:\SEQUENCE\export\EST_db\000203a\000203a-005.scf” (45>563)
GCACGAGGCCAAGAATACAGTCACCTGCAGCCGGGGGACCACCTGACTGACATCACCTTAAAGGTGGCAGGTAGGATCCATGCCAAAAAGAGCTTCTGGAGGA
AAGCTCATCTCTATGACCTTCGAGGAGAGGGGGTCAAGNTGCAAGTCATGGCCAAnCCACGAACAAATCTGAAGAAGTTTATTCGTATTAACAACAAACTGCG
CCGNNGAGACATAATTGGAGTCCCAGGCANTCCCTGGAAAACCAANAAGGNCGAGCNTAGCGTCATCCCCTATGAAATCACACTGCTGTCTCCTTGCCTGCAC
ATGTTACCTCATCTTCACTTTCGCCTCAAAGACAAGGAACACCGTATCGTCAGAGATACTTGGACTTGATTCTGATGACTTGTGAGCAGAAGTTTATCTCCGC
TCTNATAATCACGTTTTATNAAGTTCGNTGAATGGNNATTCTAAAATGTAACTCCATGAGAACATCATCCAGGGNAGCTGTGCTAGCTTTACACTACAA
>‘000203a-006.scf’ came from CONTIG 6 at offset 0;
“E:\SEQUENCE\export\EST_db\000203a\000203a-006.scf” (47>562)
GCACGAGGGTTTATTAGTGTACAAGGAGGCTTCAAGAGGGCTTCTGTGGTGACCCCGTGGTAAAGCATCTGCCTACCAGTGCAGGAGACTCCAGTTCAGTCTG
GTCTGGGAAGATGCCACACACCCGGGGGAAACTGAGCCCATGTACCACAACTGCTGAGCCTGTGTTCTAGAATCCGGGGAGCTGGCACGAGAAGTCACAGCAA
TGAGAAGCCCACACACTACTANAGAGTAGCCACACTCACCACACAAGGCTTNCCTTGTGCTCAGTTGTTAGGAATCTGCCTGCATGGCGGAGACCTGGGTCGA
TTCCTGGTCGGAAAGATCCCTGGAGAAGGAAAAGCTACCTGCCGGAGCCACACGGAAGACCCACCTGACAGTCTGTGAAGAACTGAGAGCAGGGATAAACTAG
GATCCTTGATTGTCAACTCTATCKAAACAAACTCTTCTGTTTTGTTTGTTTCACACTTCTGCGTGCAAGCTTTCCGCCCCTTTTNAAATAAATTTATTATATT
>‘000203a-007.scf’ came from CONTIG 7 at offset 0;
“E:\SEQUENCE\export\EST_db\000203a\000203a-007.scf” (32>465)
GCTGCAGGAATTCGGCACGAGGCTAGTTTCTTGTTTTTTTTTTTTTTTTTTTTTTTTTTTTTTTGCCTTTTCTTCCACTTTATTTCATATTCCCACCACAATA
ATGACTCCTTTAATTTAAACTAAAAACCATANAGGGTTCCCTGAAATTGTGGCAGCAAAGGAATGAAAGTGTCAAATACCGAGGGACAGGTGGGGTGGGGAAT
CACCGAATCGTCTCACTGGGCTCTTGAAGTTGCTGGCGGCTGAAGCTGCAGCTGGTAGGGCATTGATGGTATCTGAAACCGAAGCCTGGGCCAACCTGGTGGC
GGCCCCTTGGCCGGTACTGGGGTGCACATGAAAACATTGAAGGACCCGCGCCGCAGAAGCGCCTCCGGGGGGGGGCCTGTTGATTGGGGGTACACCCCTCCCC
TGGGAAAAAATTTCCATGGCT
>‘000203a-008.scf’ came from CONTIG 8 at offset 0;
“E:\SEQUENCE\export\EST_db\000203a\000203a-008.scf” (39>747)
CAAAATGTCTTGAAATGATATTACCATAATTTTAAGTAGGAAAGTTACCTGAACACTTCTGCTTCCACTTAACTGACTGGCCCGCAATATTGTAGGAACAGCA
TGTCCTTTGTACTGTGGTATTCAGAACAGCCACAGCACTCACTTTTTCCAAATGATTCTAGTAATTGCCTAGAAATATCTTTTTCTTACCTGTTATTTATTAA
TTTTTCCCCATATTTTTATATGGAAAAAAAAATTGTATTGAAGATACTTAGTATGAGTTGATAAGAGGAACTTGTTCTAATTATGTTTGGTGGATTATTTTTA
TACTGTATGTGCCAAAGCTTTACTACGTGGGAAAGACAACTGTTTAATAAAGAATTACTTCCCAAAAAAAAAAAAAAAAAAAAAAATAACCGGAGGGGGGCCC
GGTCCCCATCGTCCTATGGGAGCGTTACCATCCACGGGCGGCGCTTACAGCNCGGACGGGAACCCGCCGTCCCCACCTACGCCTGCACCCACCCCCTTCCCGT
GGGTAAAGGAAAACCCCACCACGCCTCCACGTGCGCACCGAGGCGAGGAAAGAAGGGTAATTTGTAATCGTAATTTTTATATATTTTTACATGCCAAGCCATC
CTTAAAAAAAAAAGAAGGGGGGGTGTGTAAAAACTCTTAAAGCCCCCGAGAAAAACCTAGGGCCCCCCCCCCCTCTTTGGGGGCGAC
>‘000203a-009.scf’ came from CONTIG 9 at offset 0;
“E:\SEQUENCE\export\EST_db\000203a\000203a-009.scf” (37>606)
TAATTCGGCACGAGGCTCATTTTTCCATCTCTTATGAGGACACACATCACTGAATTTAGGTTTACCCTAAGTCCAGAATAATCTCATCTTGAGATCCTGAAAC
TTATCACATTTGCAAAAATTCAAGAGCCAGGGAAAGCTGAGCAGTGACTGCTAATGGAACAGGGTTTGCTTCGAGGGTGATGAGAGTGTTCAGGGGTAGACAG
GGATGCTGTTTGTACGACTCAGTGAATATACTAAAACCCNAGGGATTGCATGCTTTAAAGAAGAAGCTTAATGTTTGTGAAATTAGTCTCAATATAGCTGTTA
TTTTTAAAAGAGCCTGGCTCGGGGAGCCATCAATCATACTGCTATTTTTATATCGATGTGCCAGCAGAAGTATTCTTCTTTATGACACTGTTTTACTTTGGCT
GTCTCCCACCTGGTTTAAATACATTGAACAGAACCCAGNGAAAGCCTATGGTACAGGGAGAGCCCCGCTTTGCCATGAGGGATAGAATTGGTGATGCCAGATG
CCAGAGATTCCAAGAATTTGTGAAAAACCAGACTCCGGGGCCGGGTAACCTTCTCCG
>‘000203a-010.scf’ came from CONTIG 10 at offset 0;
“E:\SEQUENCE\export\EST_db\000203a\000203a-010.scf” (44>427)
GCACGAGGAGAGAACTAGTCTCGAGTTTTTTTTTTTTTTTTTTAACTGAAGGAAAATTTCTTTACAATGCTGTGTTGGTTTCTGTCATACCAACGTGAATCAA
TCATAATTATATTATATATCCTGATGGCACATGTTAAGAATGCATTTTCTCGTTTGAACATTACTGAGTTGGGAGATATGCAGGTTATGGATTAGTCTCTCTT
GTGACTACTGACTTAACTAAAATTCAGAAGATACAGCCATTTACCTACAGTCCTCCAGTTAAAACATGGCAGACCTGAGCCTANAACCCAGTTTGCTCATTTT
GACTCCAGTATCACCCAACTATACCTAAAATGGTCCCTCTGCAGATACTATTCAAAGCACTTTATTTACTAT
>‘000203a-011.scf’ came from CONTIG 11 at offset 0;
“E:\SEQUENCE\export\EST_db\000203a\000203a-011.scf” (31>278)
CTGCAGAATTGACACGAGCACCCTCTCAAGACCGAGCTGCTGCGGGCCACACTCCTACAGTCTGTGCAAGCCCCCGAGTTCACCCCCAAGTCTGGAGGGAAGA
ACCATGTCTGTGACCAGCAACTGCAAAGAGCCAATGCCTGTGTGGTTGACAGCCGGCGTGGAGATCTCATAGCTACTCTTGCCACTGCCCGACAGCTCCCTGG
CTCAAAAATTACCCTCATCTACTTGATAAGGATGATGTACACC
>‘000203a-012.scf’ came from CONTIG 12 at offset 0;
“E:\SEQUENCE\export\EST_db\000203a\000203a-012.scf” (1>719)
TGATGCCTTCTAATTATGGTTCCCCCGGGCTGCTGGTGAGACCTGTGTATACCCCACTTTGCGCTGTGTGGTTCCAGAAGAACTGGTATATCAGTAAGAACCC
CAAGGAAAAGATGGCTCGTCTGGTACGGAGAGAGCATGACCGTCGGATTTTCAGTTCGAGTATGGCGGCCAGGGGTCCGATCCTGCCGATGTGGCCATCCAGC
TGACTTTCCTGCGCCTGATGTTCACCGAGGGTTCCATAACATCACCCTACCACTGCAAGAACAAGAGTGGCCTACATGGGACCAACTGACTGGCAACCGTCAA
GATGCCCTGCTCCTCCAGGGCTCCAACGAAGTACGAAATCCGGGCCGAGGACAACAGCCGCTCCACTACAGCGACACCTAAAATGGCTGCACGATCACACCGG
ACCCTGGGCAAGAAGAGACGAATACACACCAAACTCCGCTGCCCACATGATGGCCCCTTGAAGTGGCGCCCATACAGAATTCGTTCGAGTGGCCGCCTGTTCT
GTTCTCCTTCCCCACCGCTCCTCACCAACCCTGCCCGACTCGAAAACAACACCAACGAACCCAAAACAAAAGGAAAATCACAGCTGAAAATTTTCTGCTTTCT
TAATATTTATTACACAACTACAACAAAGACACTCAAAAAAAAACAGGACGCCCCCCCTAGGCATAATATCGGTTACGGAGGACCGCCCCCTCCTCCC
>‘000203a-013.scf’ came from CONTIG 13 at offset 0;
“E:\SEQUENCE\export\EST_db\000203a\000203a-013.scf” (284>351)
ATGAAGCGTTATATTTTGTTAAACCGTTATATTTTGTTAATCACCTCATTTTTTACCCATAAGCGC
>‘000203a-015.scf’ came from CONTIG 14 at offset 0;
“E:\SEQUENCE\export\EST_db\000203a\000203a-015.scf” (1>680)
CTGCGGCCCTCTACACTATGGATCCCCGGGCTGCAGGCGGAAGATGGCGGCCACGGCGGTGAACGGGGTGGCCGGCACCTCGAGCTCGGGGTCTGCGGCGGCC
TCGGGCGCGATCCTGCAGGCCGCGGCCGGCATGTACGAGCAGCTCAAGGGCGAGTGGAACCGGAAAAGCCTAATCTTATCAAGTGCGGNGGAAGAGCTGGGCC
GTCTCAAGCTGGTTTTGTTGGAGCTCAACTTCCTGCCAACNNCACAGGACCCAAATGACCAAGCAGCAGCTCATTCTGGCCCGTGACATACTGGAGATCCGGG
CTCAAGGAGTATCCTACGCAAGGACATCCCCTCCTCGAGCGGGACATGGCCCAGCTCAAGTGCTACTACTTCGATTACAAGGAGCAGCTCCCAGAGTCAGCCT
ACATGCACCACTCCTGGGCCTCACCTCCTCTTCTGCTGTCCCAAACCGNTGGCTGATTCCACCAGACTGGACGGTGCCTGCCAAGACATCCAACCACGGTACA
CAAGCATCGGGNCCTCGAGCATACGAGGAGGCAGTACATAGTATTCTGGCAAGCACATCCCGCGAACTACCTTCTCATGATTCGCTGAACTCAGAAGAAGITG
TGANGAAGGCATGAAATCTTTTACAAGCCCGACCCCCTCACAACCAAAAAAAAAAACCAAA
>‘000203a-016.scf’ came from CONTIG 15 at offset 0;
“E:\SEQUENCE\export\EST_db\000203a\000203a-016.scf” (8>560)
GCTCTATACTATGGATCCCCGGGCTGCAGGTTCGCTTAGGCGCAGACGGGCAAACAGAGCCAGCATGCCGGTCGCCCGGAGCTGGGTTTGTCGCAAAACCTAT
GTGACCCCGCGGAGACCCTTTCGAGAAGTCCCGCCTCGACCAAGAGCTGAAGCTGATCGGCGAGTATGGGCTCCGGGACAAACGTGAGGTCTGGAGGGTCAAA
TTCACCCTGGCCAAGATCCGAAAGGCTGNCCGGGAGCTGCTGACGCTGGATGAGAAAGACCCGCGCGTCTGTPCGAAGTAATGCCCTGTGCGGCGGCTCGTCC
GTATCGGGTGGTGGATGAGGCAAGATGAAGCTGGATACATCCTGGGCTGAAGATGAAGATTTTTTGAGAGACGCCTGCAGACCAGTCTTCAGCTGGGCTGCCC
AGCCATCACCAGCCCGGGCTCTCCGCACGCACACAGGTCGCAGCAGGGAGACATCCGTCTCATGGCGCTGGACTCCAAACCATCACTCTCCTCCTCCCCTCGC
GGGGCGNCCGGCCGGGAAGAANAAGCAAAGACAGGGGGT
>‘000203a-017.scf’ came from CONTIG 16 at offset 0;
“E:\SEQUENCE\export\EST_db\000203a\000203a-017.scf” (44>531)
GCACGAGGAGTGACCAGGGTTAGCTGGGATGCCCTCAGACTGCACTGGACCAGCCCCGATGGGATCTATGAACGGTTTGTCATTAAGATCCGGGAGACTGACC
AGCCCCAAGAAGTTCACAGTCTCACGGTTCCTGGCAGCCAGCACTCCGTGGAGATNTCCAGCCTCAAGGCTGGTACCTCTTACACAATCACCCTGCGTGGCGA
GGTCAGGGACCACAGCACTCAACCCCTTGCTGTGGAGGTCATCACAGCGGAGCTCCCCCAGCTGGGAGACTTATTCGNGACTGAGGCTGGCTGGGATGGCCTC
ANACTCAACTGGACCGCAGCTGATCAGGCCCTTGAGCACTTGTCATTCAGGCGCAGGAGGCCACAGGGTGGNAGGCGCTCAAACTCCCGGGGCCCAGGACATG
CGGCTGGGACATCCGGGCCCTGAGCGCNCCCCTACAGAGCACATCCACGGTGATCGGGCTATAGACCAGGGTCTT
>‘000203a-018.scf’ came from CONTIG 17 at offset 0;
“E:\SEQUENCE\export\EST_db\000203a\000203a-018.scf” (13>586)
AAATATGGATCCCCGGGCTGCAGNAATTCGGCACGAGGGTACCATCTNTTTTTTCAAACCTGGCAGGAATCCCCGGGGGGAAGCCCGCATACTCCTTCCACGT
TACCGCAGATGGTCAGATGCAGCCCGTCCCCTTCCCCCCAGATGCCCTCATCGGCCCTGGCATCCCCCGACACGCTCGCCAGATCAACACCGTGAGCCATGGA
GAGGTGGTGTGTGCGGTGACCATCAGCAACCCCACGCGACACGTGTACACGGGTGGGAAGGGCTGCGTCAAGGTCTGGGACATCAGCCACCCCGGCAACAAGA
GCCCGTCTCTCAGCTCGATTGTCTGAACAGGGATAAACTACATCCGTTCTGCAAATTGCTCCCTGATGGCTGCACTCTCATAGTGAGAGGGGAAGCTAGTACC
CTGTCCATCTGGGACCTGCGGCTCCCACCCGCGCATCAAGCAGACTGACGCCTCGGCCCCGCTGCTCGCCCTGCCATCAGCCGGACTCAAGTCTGCTCTCGGC
TGCAGCGAGGCACATGCTGGTGGGACTGCACACCAACGCGTGAGGCATNCAGGCACCGA
>‘000203a-019.scf’ came from CONTIG 18 at offset 0;
“E:\SEQUENCE\export\EST_db\000203a\000203a-019.scf” (13>287)
AAATATGGATCCCCCGGCTGCAGNAATTCGGCACGAGGCAGGCCTTTTTTTTCTCTCTCAGACAACCATCTCATGGACCCCATTCCAGGAAAGCTCTGAGTAT
ATCATTTCATGTCATCCAGTTGGCATTGATGAAGAACCCTTACAGTTCCGAGTTCCTGGAACCTCTGCTAGTGCCACCUGACGGGCCTCACCAGAAGGGCCAC
CTACAACATCATATGGNAAGCAGTAAAAAACAANCAGAGCAGAAAGTTCGCGAGGAGGGGGTTNCCG
>‘000203a-020.scf’ came from CONTIG 19 at offset 0;
“E:\SEQUENCE\export\EST_db\000203a\000203a-020.scf” (2>215)
CTTCCGTTATACTAAGGATCCCGGCCGCGGAATTCGGCACGAGCCTCAGTTTTTTTTTTCAGCCTCAGGCCCACCCTGAGGGTTCTCCTCCAAGCTGGCATCG
CCCCACTTTACAGATGACCACCCCAGGCTTGGACAGGGCCGCCCCTGGACAAGAAGCTGATCAAGGCCCTCTTTGACGTGCTGGCGCACCCCCAGAACTACTT
CAAGTACA
>‘000203a-021.scf’ came from CONTIG 20 at offset 0;
“E:\SEQUENCE\export\EST_db\000203a\000203a-021.scf” (29>265)
CCTGCAGAATTCGCACGAGGAGAATCTATTTTTTCTCTTGATGAGGGTGAAAGAGGAAAGTGAATAAGCTGGCTTAAGACTCAACATTCAAAAAACTAACATC
GTGGCATCTGGTCCCATCACTTCATGGCAAATAGATAAGGAAAAAGTAGAACGGGGTCAGGCTTAATTTTTTTTGGCTCGAAAATCACTGCAGATGGGGGTTG
CAGCCATGAAATTAGAGATGCTTGCTTTCTTG
>‘000203a-022.scf’ came from CONTIG 21 at offset 0;
“E:\SEQUENCE\export\EST_db\000203a\000203a-022.scf” (36>646)
AATTCGCACGAGGTGGAAGCTTTTTTGTCGGGGGTTGTGACTGGGGGCCGGAGTGCCCCACCCGATTGGTGGGTCCCCTTCCGCATTTAGGGTCCCTGAGCAT
GCTTTCTTGCCAGGGAGCTGGAAAGTTTTCTGACCCTTTTCCCCAGAAAGAGAGACAATAGATTGCCTTCATTTTGATGTCTGTGGCCTCAAAATTGATCATT
TCCTGTCTCCTCCCTCCCTCCCCGCCCTGGGGCCCCCCGCCCATTGATCCCCACCCCTCCAGAGCCACTTANGACCCACTTCTGACTAAYFATGGATTCCAGA
TGCTTGGGATAAAAGAAAAAGGACCAAGAACCCCTCCGCCTCTCTGACCTGGCCAAAGCCCTCCCCCAATCCCCAGGTCTCTGGAGGGCTCTGCTTAAGCCCG
CCTCACCGANAGNAGGNATGTAGCTGTAGAAACAACCATGCAAACTGGGTGGCCTGCAGTTTACACCACCCAATCTTCCCTCCTGGCTCCTTACATGATGAGG
ACAACTGGCTGAGAAGGGCGCAAGCGTCTGGCTCACTGCTATTTCTGAAATAGAACTGGCTCTTGCTGGCGTGGCCTGGGTAGGGCCGGCAGAGGGG
>‘000203a-023.scf’ came from CONTIG 22 at offset 0;
“E:\SEQUENCE\export\EST_db\000203a\000203a-023.scf” (1>640)
GTGCGTTAAATAGGATCCCGGCTGAGAATGCCGAGGAAAAGGCCAGGTTAAAAGGGCCCGGCCCGGCCGGGTGAAGAAGCAGGAGGCCAAGAAGGTGGTCAAC
CCCCTGCTCGAGAAGAGGCCCAAGAATTTTGGCATTGGACAGGACATCCAACCCAAGAGGGACCTCACCCGCTTTGTCAAATGGCCCCGCTACATCCGGCTGC
AGCGGCAAAGGGCTATTCTTATATAAGCGCCTGGAAGTGCCTCCTGCAATTAACGAGTTCACGGAGGCCCTGGACCGACCAACAGCTACTCAACTGCTAAGCT
GGCCCCAAGAACAGACCACAGACAAACAAGAGAAAAAGCAGAGCTGCTGGCCGAACTGAGAAAAAAGCGCGGGCAAAGGCGAGTCCCTACCAGAGCCCACTGT
CCTTCGAGCAGGTNCACACGGCCACACCTGGGGAAGACAGAAGCTCAGTGTTGTGATCGTCAGAGTGGTCCCTTGGCTGGGGTCTCTGCTGCCTGGGCGCAGA
GGGGNTTCTATGCTATAAGGCAGGCGGCTGGCGCGTGCCAGAGACGGCCACGACTTACCATGACGGGAAAGAGCTGTTAGGGGAACCAGACATTAAACAAAAA
GAACGGGTGGGGGAGGCGGGCA
>‘000203a-024.scf’ came from CONTIG 23 at offset 0;
“E:\SEQUENCE\export\EST_db\000203a\000203a-024.scf” (1>602)
CGCCGCCGCTTAAACTATGGATCCCCGGGCTGCAGNAATTCGGCACGAGGATTACAAGCGGATAGAAGGGCTAAAAATCAAAGGCGAAGAGTTCATATGACTC
TGGAGTTACCATGGAGTGCTGATAGAGCAATTCAGCAATTTGGACGAACTCATAGATCAAATCAAGTTACCGCTCCTGAATATGTCTTTCTGATTFTCTGATT
GGCAGAAGAACAAAGATTTGCATCTATTGTTTGGTAAAAGACTTGAGAGTTTGGGCGCACCTACACATGGAGACAGAAGAGCAACAAAAACTAGAAAACCGAG
CCGCGTCCACCTTCGATAATAAGATGGAAGAAAAGCTTTAAAAATTGTGATGAAATCCAATGTGAAACCAAATTCTTTCCTTGGTTCACCACTCCAGACTATC
CTGGAGATTCTTTAAGAGTTTCGCAAGACTGATAGAGTGTCTTATAAAGTGAAAAAGTCAGAATCTTCTTTTATAAAATTTAAAACAAAGNAAATTTTAACAA
TTTGGGCGGGAGGGCCCACAAAGCCTTTTTTTTTCGCACCCCTCCTGCGGCTCAAAGCAAAAAAGAAAAACATAGGAATTAATGTTGTTG
>‘000203a-025.scf’ came from CONTIG 24 at offset 0;
“E:\SEQUENCE\export\EST_db\000203a\000203a-025.scf” (29>176)
GGGCTGCAGCTCCATGGGGTGTTGGTGCCTGCCAGCCACGGAGGCCGGGCGGCCAGAACGCGCACAGAGGGATATGATATGGTCCGGTGTGATGGAGAGAGCA
AGCGGGACCGTGCAGCCTCCCAGGACACTGGCCCCGCGGGGAGCC
>‘000203a-065.scf’ came from CONTIG 25 at offset 0;
“E:\SEQUENCE\export\EST_db\000203a\000203a-065.scf” (1>665)
GTGGCGCCCTCTAAACTATGGATCCCCGGGCTGCAGGTGGACTACACCATCACTGTCTATGCTGTCACCGGCCGGGGGACAGCCCGGCAAGGAGCAAGCCCGT
TTGCATCAATTTACCGAACAGAAATTGACAAACCATCCCAGATGCAAGTGACTGATGTCCAAGACAACAGCATTAGTGTCAGGTGGCTGCCTTCAAGTTCCCC
TGTTACTGGTTACAGAGTGACCACTGCTCCTAAAAATGGCCCAGGACCATCGAAAACGAAAACTGTAGGTCCAGATCAAACAGAAATGACAATTGAAGGGCTG
CAGCCCACAGTGGAGTATGTGGTCAGTGTCTATGCTCAGAATCAAAACGGAGAGAGTCAGCCTCTGGTTCAGACAAGCGTTACCCACCATTTCTGCACCAACC
AACTGAAATTNACTCAGTGACACCACCAGCTGACTGCCAGGACGCACCNATGTCACTCACTGGTTCGAGGCGGNGACCCGAGAAAGACGNACGAGAAGAATCA
CCTGCTCTGAACTATCGGTTGTTCAGACTAGTTGCACCAATGAGGAGGCTTGCTTTAGACCTGACACAACGCTAGGAGGTCAATTGAAAGCACTCAAAGGCCG
GGAAAGTTGAACACTCCTTATGAACAAATGAAAACGGTCAGTGGCTGC
>‘000203a-026.scf’ came from CONTIG 25 at offset 0;
“E:\SEQUENCE\export\EST_db\000203a\000203a-026.scf” (38>628)
AATTCGCACGAGTGTCTATGCTGTCCCGGCCGGGGGACAGCCCGGCAAGCAGCAAGCCCGTTTCCATCAATTACCGAACAGAAATTGACAAACCATCCCAGAT
GCAAGTGACTGATGTCCAAGACAACAGCATTAGTGTCAGGTGGCTGCCCTCAAGTTCCCCTGTTACTGGTTACAGAGTGACCACTGCTCCTAATAATGGCCCC
AGACCATCGAAAACGAAAACTGTAGGTCCAGATCAAACAGAAATGACAANTGAAAGCTTGCAGCCCACAGTGGAGTATGTGGTCAGTGGCTATGCTCAAAATT
CAAAACGAGAGAGTCAGCCTCTGGGTGAAACAGCGGAACCACCATTCCTGCACCACCCACCTGAATTACTCAGNGAAACCAACAGCTGACTGCCCAGGNACGC
ACCCATTTCACTCACTGTTCGAGGCGGTGACCCGAAGAGAGACGNACGAGAAAAATCACGTGCTCTGAACTATCGGGTGTATAGACTAGTTGCACAAATTAGG
AGGCTAGTCTAGACCTGAAGCAACGCTCAGATGTACATTGAAAGAGCCTCAAAGCCGGGAAAGCTTGAACCCTACA
>‘000203a-027.scf’ came from CONTIG 26 at offset 0;
“E:\SEQUENCE\export\EST_db\000203a\000203a-027.scf” (30>646)
CTGCAGAAATTCGGCACGAGGAGCGAGTCGTGTTAGGTGCGCGTGGAAACTAGGGTCATGGCTGCGCCCGGTCCAGCGCTCTGCGTTTTCGACGTGGACGGGA
CCCTGACGGCCCCGCGGCAGAAAATTACCAAAGACATGGATTGCTTTCTGCAAAAACTGAGGCAGAAAATCAAAATTGGTGTCGTCGGCGGGTCGGACTTTGA
GAAAGTACAGGAGCAGCTGGGAGATGACGTTATTAAAAAATATGATFACGTGTTTCCAGAAAATGGCTTGGTAGCATACAGAGATGGGAAACTCTTGTGTAAA
CAGAATATTTAAGGTCACCTGGGTGAAACCCTAATCAAGATATATTCACTACTGTCTGAGCTACATCGCGAAAAATCAGCTCCNGAAAAAAAGGNCACTTCAT
AGAGTCCGTAACGTGAGCTGACGTGTCGCCGACGGAAAAGCTGCAGCAGAANAACCATGTATCTACGACTGTACAAAAGAAACATAAACAAAGTCGGAGNATT
GCAAAGATTGCTGTAAGGCTACGTTTCTAGAGNCAATCACTTATTCTCCCTAGCTGAACAATACGCTGGAACGGGAAGAAGATAAACTTATTTTTGGACAAC
>‘000203a-028.scf’ came from CONTIG 27 at offset 0;
“E:\SEQUENCE\export\EST_db\000203a\000203a-028.scf” (40>622)
CACGTAGGGGCGACCGCAGGCCCTCTCCCGAGGAGCTGGACAAGGGCATCGACCCCGAGAGCCCCCTGTTTCAGGCCATTGGACAACCCCGTGGTGCAGCTGG
GCCTGACCAACCCGAAGACCTTACTAGCATTTGAAGACATGCTCGAGAACCCGCTGAACAGCACCCAGTGGATGAACGACCCGGAGACGGGCCCGGGCATGCT
GCAGATCTCAGAATCTTCCAGACCCTGAACCGCACATATGCCGCGCACTGCAGCTGCCAGCCCAGAGAGCCTCTTCTTCCCAGCCCAGGGGTGGGGAGAGGGT
GCAGACCCCAAGGTGCGCCTGGGCTGGGGGCGGGGAGCAGGGGGGCNTGGAGGGACCCTGCCCCTGGGTGTGGCGCCAGGCCGCACTCCGCTGGATCTTCTGG
AAAAACTCGGNGGCAGGGCCGGGTGGCTCCACCCCTGACAGGTTACGACAGGCGCCACCGGGAAGGGGGCTCCTTCAGGCCCTGGCTCTGACGTATTGATTAA
CGAGCGCGCTGGAAGACCTGTTTGAAAAAGAATGTCAACCAGTTAGGAAGGATAATGGGAAAAAAA
>‘000203a-029.scf’ came from CONTIG 28 at offset 0;
“E:\SEQUENCE\export\EST_db\000203a\000203a-029.scf” (35>595)
AATTCGCACGAGGTCATCCCTAAGTGGCCTGAAGATGGACAAAGGGAAGTAACAGGCACGTGATGTTGGCAAGGATGCTTCTAGGGCTAGAGGATCAGTGGTG
GGAGAGAGCTGCAGAATCCACCAGCCAGAACTGCAGATAACGATATCTATGGTCAGGGGCTGTGACTGAGAGAAGGAAACTGAGGTTGTGTTCTGAAAGTACA
TAAACTCTCACATATACCCAGTTCTTCACCATCTTCCCTCCTCACTTTGCAGNGCCATTTTTTTTTTGCATITAGGCAAATTGCTCAGACTTTCCAGAGCCAT
GCCCATCCCGTCTCTGGAACCCCCACACCTCTGAGAGTGGGATCACCACGTCCTGCAGGGCTGCTCCCCTCCAACTACCTTTAGAGAGCAGGACAGGAGCTGT
TTCACCACAAGACAAAATCAAACGAGAGCAGACGGGTAAACAAANAAGACAGGGGCAATGTTTTCTTGNGTTTTGTTTTTTTTCCATTGGAGGTGACACAAAA
ATTCAAGCTACAGTTCCCCTCTCCCCCCCATTTTTTTTTAACAAANA
>‘000203a-030.scf’ came from CONTIG 29 at offset 0;
“E:\SEQUENCE\export\EST_db\000203a\000203a-030.scf” (36>676)
CTCATCCTACCATATAGATATTGGTACCCTTTATCTACTATTTGGTGCTTGGGCCGGTATAGTAGGAACAGCTCTAAGCCTTCTAATTCGCGCTGAATTAGGC
CAACCCGGAACTCTGCTCGGAGACGACCAAATCTACAACGTAGTTGTAACCGCACACGCATTTGTAATAATCTTCTTTCATAGTAATAGCAATCATAATTGGA
GGATTCGGNAACTGACTTGTTCCCCTAATATTTGGTGCTCCCGATATAGCATTTCCCCGAATAAATAAATAAGCTTCTGACTCCTCCCTCCCTCATTCTACTA
CTCCTCGCATCCTTATAATTGAAGCTGAGGCAGAAACAGCTGAACCGNGNACCCCTCCTTANNCAGCAACCTACCATGCAGGAGCTNATAGAACTACCATTTC
TTTTCACTTACANGAGTCCTCATTTTAGAGCATCAACTCTTACACAATACAACATAAGCCCCGCATGCCATACAACCCTTGTGTTGATCGNATATACGCGACT
ATATATTGCTCTITTACACGCACCAGCTTTAAACGAACTATCACTGTCACGCGAGAGAACTTTTTTACATTTTGTTTTGGCCCGAGCTTTTTTTCTGGGGGAT
TCTTTGCCCCTCAAAAAAACAGTA
>‘000203a-031.scf’ came from CONTIG 30 at offset 0;
“E:\SEQUENCE\export\EST_db\000203a\000203a-031.scf” (13>195)
TACTATGGATCCCCGGGCTGCAGGNAGTTTTTTTTTTTTTTTTTTTGTACAAATCAAGCATTTTATTACATAAATAAAAGCAGCACGCTTTTATTTTCTATTT
AAATACCATACACGAGATTTAAAATCACATTTGGCAGTGGACTGCAGGATGCTCAGACTTCACCCACATCACNTTGGATT
>‘000203a-033.scf’ came from (F3, 033) no description length
779GGCGCCCTCTAAATATGGATCCCCGGGCTGCAGGAATTCGGCACGAGGCCGGACGGGTGTCCTTCTCTGGAGGCTCCTCGCTGGTCGTGGGGGAGCCGGG
AGGGCATGGCTGGCTGCCCGAAAGAGAGTGCGAGACGGTGACCTGCTGTCTCTTTTCGGAGCGGGACGCCGCCGGAGCTCCCCGAGAAGCCGGCGAACCCCTG
GTCGGGGCGGCCCTAGAGCCAGAGGCGGTGGGCGGGAGCGCGAAGCCCGCTCGCGGGTGCTGCTGCTGTAGCAGGAACTCAAGATGGTCACGATCTCGCTGCT
GAAGCGGCTCAAGGAACGCTCGTTGGCCACGCTGCTGGAGGGGGGAGACCCGCGGGGGGGGCCGGGCGGCTGCGGGCTGTGCCCGCCGCCGACCTCCCCTGGC
GGCCACCCCGCCGCGCACTGGTGTCGGACCCCCTGCCTGGCCCACCTCAGCCGCCGGGGCCCAGCCCTGGCGGGGTCCACTCCCCCCCCCCGAGGCCCCAGGG
CCGCACCCCACGCTCACCGCTCGGGGCCAAATACCGGCCGCCTACCTGTTTCCCCGGACAACAACACGATTATTATCCCTTGCTAATGAAAAAGGCCCCCTTT
CATCCGGAATCAACCACTGCCGCCCCCACCACCTGGCGGGGCTGTACGGCGGGGGGCTCCCTCCCCCCCCCCATTTCTCTCTTTTTCTCATTACATTTGGGGG
CTATATATATAATATATTATTATAGATATTATTTTTTTTTCTATCTATATTTTTA
>‘000203a-034.scf’ came from CONTIG 31 at offset 0;
“E:\SEQUENCE\export\EST_db\000203a\000203a-034.scf” (28>623)
GGGCTGCAGAATTCGCACGAGGCTGTCTGCTCGTGGTGGAGATGGCAGTAGGATCATTTTTTGATGATTTTCGAGAAGCGTACTATLTGGCTTCGTCACAATA
CTCCAGAGGATGCGAAGGTCATGTCATGGTGGGATTATGGCTACCAGATTACAGCTATGGCGAATCGGACGATTTTAGTGGATAATAACACGTGGAATAATAC
CCATATATCTCGAGTAGGGCAGGCCATGGCATCCACAGAAGAAAAAGCCTATGAGATCATGAAGGAGCTTGATGTCAGCTATGTGCTGGTCATTTTTGGNAGC
CTCACTGGGATTCTTCAAATGACATCAACAAATTTTCTGTGGATGGGCCGGATTGGAAGGAGCACAGATACAGGAAACACATACAGGACACGATATTATACTC
CACTGGNGATTCGNGTGGACCCGGAGGCTCCCANGCTGCTCACTGCTTTAGACAAAAGGGTACTACGATTGACAGGNTACCAAAACCACGGCCCTAGCTTTAC
CGGCCGGATGCGAGATGGAATAAACTCGACTGAGTCTAAAAACAACACACCACATGCTGGCGAATTCAAGGAGACCGNAATCAG
>‘000203a-046.scf’ came from CONTIG 32 at offset 0;
“E:\SEQUENCE\export\EST_db\000203a\000203a-046.scf” (37>597)
NAATTCGGCACGAGGGCATGAATGTCCTGGCCGATGCTCTCAAGAGTATCAACAATGCCGAAAAGAGAGGCAAACGCCAGGTCCTTATTAGGCCGTGCTCCAA
AGTCATCGTCAGGTTCTAACAGTGATGATGAAGCATGANTACATTGACGAATNTGAAATCAYPGATGANTCACAGAGCTGGAAAATTGGTGNGAACCTCACNA
GCAGGGCTAATAAGTGTGGAGNGATCAGCCCTAGATTGATGTGCAACTCAAAGATCTAGAAAATGGCAGAATACCTGCTCCATCCCGCAGTFGGTTTCATGTA
CTGACAACTCAGCTGCATCATGGACATGAAGAGCAAGACGAAACATACAGAGGAAATCTTGATTCTTTTTAGGAGTATACTACAATAATGCTCAAGACTTGTG
CTTCTTAAAAAAAAAAAAAAAACGACCGCACTGATGACAGTCTACATATTCTGACCTTTTATCTCACTAAAGTCAACCACTTTTCCATCAACGAACACAAAAT
AAAAAAAACCCTGAAAAAAAAAAATTTTTTTTTTTTCTTTTT
>‘000203a-035.scf’ came from CONTIG 32 at offset 542;
“E:\SEQUENCE\export\EST_db\000203a\000203a-035.scf” (37>597)
TTTTTTTTTTTTCCTTCTCGCTCCCTTCCTTTCTTCCTTACTTACTTCTTTTGCTFFTGGCTGCATTTTCTTTAAATTCGACACAGTTATGTTAAAAAATATA
TGCATTGTACTTAGAGTTTGGTGTAATTTAAAATATGTGGAGTGATTTCATTCACTCTCCTGTTTTAAACATTTGTTAAGGACTCAGCATGTGAAGGAGCAAG
AGATATAGTCATTTTTATTAGAAAACTTCAGTGTTCTAATTTCATCAGAGACCGNGAATAATCAGAAGATGACATGATTTACTGGAATATACAGCTTATCAAG
GACTTCGTTATTTATGATGGTTATTTAAAATC
>‘000203a-037.scf” came from CONTIG 33 at offset 0;
“E:\SEQUENCE\export\EST_db\000203a\000203a-037.scf’ (37>554)
CGCAGCCACTCCGACCGGTGCCGCCTCGTCCTGCTTCGCCATGACTTCCTACAGCTATCGCCAGTCGTCGTCCACCTCGTCCTTCGGGGGTATGGGCGGCGGC
TCCATGCGCTTCGGGGCTGGGAAGCGCCTTCCGCGCGCCCAGCATCCATGGNGGCTCAAGTGGCCGCGGCGTGTCGGTGTCCTCCGCCCGCTTCGTGTCCTCG
TCCTCCGGGGGCTACGGCGGCGGCTATGGGGCGCCCTGGCCACCTTCGACGGGCTGCTGGCGGGCAACGAGAAACTCACCATGCAAAACCTCACGACCGCCTG
GCCTCCTACCCTGAGAAGTGCGCGCCCTGGAGAGCCAACAGCGATTGGAGTGAAAATCGCGACTGGACCAAAACAAGGCCGGCCCGCCGCGACTACACCTACT
CAAACATAAGACTGCGNACCAACTCGTGGCACATGAAACTCATAATCTGCATACACAGCCGTCGCTGCAAGACTCGCACATTTGAGACGACAGCTGGCAGAGG
GAGC
>‘000203a-038.scf” came from CONTIG 34 at offset 0;
“E:\SEQUENCE\export\EST_db\000203a\000203a-038.scf’ (38>594)
NAATTCGGCACGAGGAGCAGATCCTGGCTGCCCTCGAGAAAGGCTGCAGCTTCCTGCCGGACCAGTACCGCAAGCAGTGTGACCAGTTTGTGACGGAGTATGA
GCCAGTGCTGATAGAAATCCTGGTGGAGGGGATGGACCCTTCCTTCGTGTGCTTGAAGATTGGAGCCTGCCCAGCAACCCACAAGCCGCTTTTGGGAGCTGAG
AAATGTGTCTGGGGCCCGACCTTACTGGTGCCAGAACATGGAGTCGCAGCCCTGTGCACCGCGTCGAGCACTGCAGCGTCAGGNGTGAAACTAGGGCACGCTT
CACCCTGAAAAACTGCAGCGTCTTTTCTGCTCGGTTGTCTGGGGTAACCACACCAATTGTGACTTTGTATAAAAAAGACCCTTCCTCATCCTTNTTCTCCCTC
TTGTGCGTGCTTGCAGGCAGTGACTGCTGTTTGGTCCTTTTTGTAAAAAGCGAACCTCCTGAGTTTTGATTGTGGCGGGGTAGGGGAAAGGGTTGTCGGAGGA
ACGACCTCGCGAGGCCGCCCCGCTGTTGGGGGGGCCTGCGCT
>‘000203a-040.scf” came from CONTIG 35 at offset 0;
“E:\SEQUENCE\export\EST_db\000203a\000203a-040.scf’ (29>585)
GGGCTGCAGGTTAATTCATTTTTCTGGAAAAAGAGAAGATGTTTATTTATTTATTTTTCCATGGTAAATTCTTTTGAATCTGCCTCTTAAACCTAACTCTGGG
CTCTCTCAGGAGGGGCAAAGAGGACCTTTGAGTTAAACCCTCCAATGGAGACCCTGGGAAAGAACCGGAGGCATAACACCCNAGCCGCCCTCCAACTGGACTG
TANGACTCCCCAGACCCGCTGCCCAGCTGCTTCTGCCCATCGNTCTGCCTGGTTGGGTTNTGGGTCCTGGATCCCACCCGAGCCCTGTAGGATGGCACCACAA
GCCCTACATGAAGAGCTTTGTGGTGTCACTAAAATGTGTGTTTCGGCACGTTGCTGTCATTCTGCCTGNCTGCCATGCTGAAAAGCTGGCACAGCCCGANAAG
CCAGCGAAAACACCTTCTGCCAGANCTCTGNCCCACTCGAGATGAGACCACCAGGTGCTGTCCTCCCAGAACAGGTATTATATTTAAGTAAAACTGTTACTAA
AAAGTTTGTTCCAACTTATTCAAAAGAAGAGAAAAGGGGCGT
>‘000203a-041.scf” came from CONTIG 36 at offset 0;
“E:\SEQUENCE\export\EST_db\000203a\000203a-041.scf’ (1>593)
GGGGCCCTCTAAAGTATGGATCCCCGGGCTGCAGNAATTCGGCACGAGGCGGACCTGCTGGAGCTCCTGGCACTCCTGGACCTCAGGTATTGCTGGACAGCGT
GGTGTGGTCGGCCTGCCTGGTCAGAGAGGAGAAAGAGGCTTCCCTGGTCTTCCTGGCCCCTCTGGTGAACCCGGCAAANCAGGTCCTTCTGGAGCAAGTGGTG
AACGTGGCCCCCCTGGTCCCATGGGCCCCCCTGGATTGGCTGGACCCCCTGGCGAGTCTGGACGTGAGGGAGCTCCTGGTGCTGAANGATCCCCTGGACGAGA
TGGTTCTCTGGCGCCAAGGNTGACGNNGTGAGACCGNCCCTGCTGACTCCTGTGCTCCTGCGCTGCCGGGCCCCCGNCCCTGTCGACTGCCGCAGACGNNGAC
GGGTGAGACGGCCTGCTGTCTGCTGTCCCATGCCCGTTGTGCCGGGCCCGTGNACCCAGCCCCCGGGACAGGTGAAAGCGACAGGGACGAGCATAAGTCACGN
GCTCTTGTCTCAGTCCCCGCCTCCGCTTCTGGAGCAGTCTTCGACTTGTCTGTGGCCGCGCCCCGTTTTCTCGCA
>‘000203a-047.scf” came from CONTIG 36 at offset 42;
“E:\SEQUENCE\export\EST_db\000203a\000203a-047.scf’ (41>205)
CACGAGGACGGACCTGCTGGAGCTCCTGGCACTCCTGGACCTCAAGGTATTGCTGGACAGCGTGGTGTGGTCGGCCTGCCTGGTCAGAGAGGAGAAAGAGGCT
TCCCTGGTCTTCCTGGCGCCTCTGGTGAACCGGGCAAACAAGGTCCTTCTGGAGCAAGTGGTGAACGTGGCCCCCCTGGTCCCATGGNNCCCCCTGNATTGTC
TGGACCCCCTGGCGAGTCTGGACGTGAGGGAGCTNCTGGTGCTGAAGGATCCCCTGGACGANATTGTTCTCCTGGCGCAAAGGTGACCGTGGTGAGAACCGGC
CCTGCTGACCCTCTGTGCTCCTGCGCTCCCGTGCCCCCGNCCNTGTCGACTGCCGCAGACGTGATCGTGGTGAAACAGGCTGCTGTCTGCTGTCCATGNCCNG
TGTGCCNGGNCCCCTGNACCCAGACCCGGTGACAGGGAAAAGCAACAACG
>‘000203a-042.scf” came from CONTIG 37 at offset 0;
“E:\SEQUENCE\export\EST_db\000203a\000203a-042.scf’ (1>652)
CGGCGTCCCTCTANACTATGGATCCCCGGGCTGCAGTGGTTCTGCAGCTCTGTGGCAAGCCGCGGAGTCTGGGTTCTGATCCGCAGGATGGGGTTTGTTAAAG
TTGTCAAGAACAAGGCCTACTTCAAGAGATACCAAGTGAAATTCAGAAGAAGGCGAGAGGGCAAAACTGACTACTATGCTCGGAAACGATTGGTAATCCAAGA
TAAAAATAAGTACAACACACCTAAATACAGAATGATTTGTTCGTGTAACGAACAGAGATATCATTTGTCAGATTGCTTATGCCCGTATAGAAGGAGATATGAT
AGGTTGTGCAGCTTATGCTCACGAACTCCCAAATATGGNGTGAAGGNTGGCCTGACAATTATGCTGCGCATATTGTACTGGCCTGCTGCTGCCCCGCAGCTTC
TTTATAGGTTGGATGGACAAAATTATGAAGCNAGACGAGGNGATGGAGAGATACATGNGNAAGCATCGAGCCAACTGGGCCTCACTGTACTGNAGCAGACTGC
AAACTCTACGAGTTAAGTTTGGCCCTAGGACGCGAGAGCTGCTTTCTACAGACACGTCCTGTTGATCAAAGCAAATCAGCGAGCCCGAAGCATAGGCAAAGTG
AATACGCCCCTGTGGAAAAAATCGAAAACTTTTCA
>‘000203a-044.scf” came from CONTIG 38 at offset 0;
“E:\SEQUENCE\export\EST_db\000203a\000203a-044.scf’ (1>627)
CGGCGCCCCTCTACAACTATGGATCCCCGGGCTGCAGNAATTCGGCACGAGGCAGGACAATCAAGTGTGGCAGCTGGGCTCATCGTCCCCAAACTTCACTCTG
GAGGGACATGAGAAAGGTGTGAATTGCATTGATTACTACAGGGATGGTGACAAGCCATACCTCATCTCTAGAGCAGATGACCGTCTTGTGAAAATATGGACTA
TCAGAATAAACTTGTGTACAGACACTGGAGAGGACATGCCCCAAATGTGTCTTGTGCCCAGTTCATCCTGAGCTGCCCATTTTATCACAANTTCAAAAAATGA
AACTGGCGTTTTGGCATTCAAGCACCTTCGCCTTGAGAGGACTTGATTATGAATGGAGAAGAGATGGGGGGGCCGCCGCGAGGTCCATAACGTGTTTTGGCTT
TGAGAAAGAAGCATATGTTAACTTGTCGGAGAACTGCTTGTGTGGTGCCAAGGAAAATAATGGGCCAACATCAAAATCACCAGCCACTAAACAAGGAGAGTGT
AATTAAAGAAAAGATGCCTGCATAAAAATGCAGTFGAATTACTCAATATCACAATCTAAGCGCGGTGGGGGGGGGGGGGAATTTTTCCGGAGCCCGAAACAAT
GTGGGCCG
>‘000203a-045.scf” came from CONTIG 39 at offset 0;
“E:\SEQUENCE\export\EST_db\000203a\000203a-045.scf’ (44>624)
GCACGAGGCTTGCCTGCTGCCTGCCTGCCTGCCACTGAGGGTTCCCAGCACCATGAGGGCCTGGATCTTCTTTCTCCTTTGCCTGGCCGGGAGGGCCTTGGCA
GCCCCTCAACAGCAAGCCTTGCCTGATGAGACAGAAGTGGTGGAAGAAACCGTGGCCGAGGTGGCCGAGGTACCCGTGGGAGCGAACCCCGTCCCAGGGGAAA
TAAGAAGAATCGATGATGGTGCCGAGGAAACCGAGGGGAGAGTGGGGANCGAGAACCCCCGCCAAACCACCACTGCAACACGGCAGGNGTGTGAACTGAACGA
GAACACACCCCATGGTGTTGGCCAGACCCCACCACTGCCCTGCCCNTCGCGAAGTTGAGAGTGTGCACAACGACAACAGACTTCGATCCCTGCCATTTTTGCA
CNAGGNACATGNAGGCACCAAAAGGCCCAACTCACTGNCTACTCGGCCTGAATACATCCCCTGCTGCATCGACTGATGATCCTGGCTGCGACGCTAAACGCCG
NCACTGACAAGGAAGCACACTCGACGAAAATATGGATGAAAAACCAAATAAAGCCGGGGGGGCCTC
>‘000203a-048.scf” came from CONTIG 40 at offset 0;
“E:\SEQUENCE\export\EST_db\000203a\000203a-048.scf’ (38>559)
TTATACTCCAAGGCCTGGGAAAATCACATAATCAAGATTGAATTGTTTCAGAAATATTGGCAGGATTCTTGGACTGTGTCTACTACAGAATGAACTGTGTCCT
ATCACATFGAATAGACATGTGAHAAAGTGTTGCTTGGTAGGAAAGTCAATTGGCACGATTTTGCTTTTTTTGAGCCTGTGATGTACGAGAAGTTGCGGGCACT
TATTCTTGCTTCTCANAGTTCAGATGCTGATGCTGTTTTCTCAGCAATGGATTTGGCATJTGCAATTGACCTGTGTTAAGAGAAGAGGGGAGACAGNTGAACT
ATTTNCTATGTGTAATATACCAGTCACTCTCAAATGTTATGAGTATGTGCGGAATATGCTGACATAAATGTNNGTAGTGCAGACACCATACTGCATGAGAAGT
CTCTGNTGTGCTTCAAAATCATATANATTACACAGAAATTAGCTTTGTTAGCTGGNGAGNTACGGCGAGCGTCAGTCACTCTTCTGAGATAGAAAAGTGAACT
TGCGTC
>‘000203a-049.scf” came from CONTIG 41 at offset 0;
“E:\SEQUENCE\export\EST_db\000203a\000203a-049.scf’ (1>306)
GGGCGCCCTTTAAATAGGATCCCCGGCCTCAGGGTGGCAAGAGGCCGTGCTATTTTTTTTTTTTGTAGAAGTTTGTCGCTGATGGCATCTTCAAAGCTGAACT
GAACGAGTTTCTCACTCGGGAGCTGGCTGAAGATGGGTACTCTGGAGTFGAGGTCCGAGTTACACCAACCAGGACAGAAATCATTTATCTTGGNCGACCAGAC
ACAGAATGTACTTGGTGAGAAGGGCCGGCGGATCCGGGAATTGACTGCTGTGGTTCAGAAGAGATTrGGCTTCCCTGAAGCAGTGTAAAGCTTATGCTGAAAA
>‘000203a-050.scf” came from CONTIG 42 at offset 0;
“E:\SEQUENCE\export\EST_db\000203a\000203a-050.scf’ (39>525)
NATTCGGCACGAGGCTAGTCTCGAGTTTTTTTTTTTTTTTTTTTCTTTGGAAAACCAAACATGCTTTATTTCATTTTTTTTCACAATTTATTTAAACATCTCA
CATATACAAAATAGGTACAATTTTAATTTTTCTGCTTGTCCGAGAAACAAGACTTCTTTGGAACCATGGNAGAGGATGAAAATGAGACTGGCAAAGAACAAAT
GCTGAANTTAAGAAGAGACAANTGTGGGCAAATGATCCACTTACTTTTGTGGAATAAGATGTAAAGTACTGATGTTTAAAGTCAAATGAAAAAAATACACAAT
ACAGCTCAACAGCAGAGGAGTATCTCTTCTCAAATTCTCCTAGCACCATCAACATTCTTNCAGTATCTGAAATACTGTTAATTAGCACCTTCGTATTTTGAAC
NAAAAAACACAAATACCTCAGCTCATCTCTGGTCAGCACTCACGGTGTGGTATCACACTCACAGGAAANGTTTTGA
>‘000203a-051.scf” came from CONTIG 43 at offset 0;
“E:\SEQUENCE\export\EST_db\000203a\000203a-051.scf’ (38>406)
NAATTCGGCACGAGGATCATATAGTAAACCCAAGCCCTTGACCTCTTAGAGGAGCTTTGTCTGCCCTCTTAATAACATCCGGCCTAACCATGTGACTTTCACT
TTAACTCAATGACCCTGCTAATAATTGGCCTAACAACAATATACTAACAATATACCAATGATGACGAGATGTTATCCGAGAAAGCACCTFUTCCAGGGGCACA
TACCCCAGGTGTCCAAAAAAGCCTCCGTTATGAATATTCTTTTTATTATCTCCAAGTACTATTCTTTACCGATTTTTTTGAGCTTTTACCACTCAGCCTCGCC
CCACCCCTGACCTAGCGCTGCTGACCCCCACACGGATTCACCCACTAACCCCTACAAGTCC
>‘000203a-053.scf” came from CONTIG 44 at offset 0;
“E:\SEQUENCE\export\EST_db\000203a\000203a-053.scf’ (37>515)
TGAGAGCAGCAGCCAAAAACCACGCTCGAGTGACAGTAGTATGTGAGCCGGAGGACTATGCAGCTGTAGCCTCAGAGATGCAGGATTCTGACAGCAAAGACAC
GTCCTTGGAGACAAGACGCCAGTTAGCCTTGAAGGCTTTTACTCATACAGCACAGTATGATGAAGCAATTTCAGATTACTTCAGGAAAGAGTACAGTAAAGGA
GTATCTCAGATGCCCCCTGAGTATGGAATGAANCGTCATCAGACTCCTGCCCAGCTGTATACGCTGAAGCCCAAGCTCCNTTATCACAGTCTGAATGGAGCCC
NTGATITATAANCTGGGTGATGCTTTGAATGCCTGCAGCTGGTGAAGGAACTCNAAGAGCTTTTAGCTTNCACTGCTGCGTCTTCAAACATGTAGCCCACAGG
CTGCTGTTGGATTCACTCATGAAGAGAAACCACTCTGCATGTTATGATTGTACAAACCTCCACCGCA
>‘000203a-054.scf” came from CONTIG 45 at offset 0;
“E:\SEQUENCE\export\EST_db\000203a\000203a-054.scf’ (40>404)
CCTAAATTACTCAATAGTTTTAAAGTGTTACATATTCAAAGCCTTTTCCAGACCAGGGAGAGAGTTCTGTTAGAGTGAAGGGTAGTGTCTCTTGCGCATTCTG
TGTGTGTATTTCTAAATGCTACTGTGTGTGTTTGTGTGTGCTCCCACAGTTTATATGCAAAGACTFTGAGCAACATTTATAAAAAGTATTTTCTCTTAGAACA
ATTCAAGAGATTTLATTTTGTGGCTACCACAGNCACTGCCAGTGGATTGTTTTTTCTTCTAAATCTGAATATTGACCAAAAATTTGGTGATTTTTATGACTTG
TGTTGGTGTTTAATTTTCTTAAAAATTTAAACTTGGTTAAAATTCAGAAATC
>‘000203a-055.scf” came from CONTIG 46 at offset 0;
“E:\SEQUENCE\export\EST_db\000203a\000203a-055.scf’ (1>728)
GTGCCTCTCCCTATACTATGTATCCCCGCGCTGCAGTATATTTTGCATGTAGGAGAACATGTAATCTCTATCTTCCCTTAGCAGGATCAAACCTAGGGCCTTC
TGCATTGCGAGCACAGAAGCCTATCCTCTGGACCACTTGGGGGAGTCCCCGCTTTTCTTTGCATCCCAAAGAATATTATAACTAACCTAAAGAAAACCGCATT
TTCCCCTTATCGGCGCGCTTCTTTCCTTTAGTCTAAGACAATAATGTCTTATTGTCCTGGGGGGGACATAGTCAGTACGGGTTAAGCTCCTCTAACCTTTGGT
GGCATTTTTTGCCCCAAATATTGCTTTTCCAAAAACCACAGAGGCTGTTTCTTTTATTAAATTCCTTCTGGCGCGCCCCACTTGGGGGTGGGTTGGCCTCTTG
GGTCTTTCTTAATAATAAAACAAAACGGTTTGACTGTGTCTCCCCCAGGTACTTTTTTTCTCTCTTTCAGAGTACTGTGAACCGGACTCCAGTTTTCTCCTGG
GACGTCAAATTTTCCTACTCTCATCGCCTCTGCTGTGGCCTTTTCCATTTCAAGAAATTCGACCTATTATTCACTCCTTAAAAAATATATGATGCCCCGCCCC
TCTACTTGCTTCGCGACAGACACAACAATTTTCTTCTAAGCAGGCGAACCACAACAGAATAGGAAGATATCTATACAGAAGGAAAGAACGTATTCATGGCGAT
CT
>‘000203a-056.scf” came from CONTIG 47 at offset 0;
“E:\SEQUENCE\export\EST_db\000203a\000203a-056.scf’ (17>140)
TCAGGGATCCCCGGGCTGTCAGCTGCGTCAAGTGTTTGTCATGAATGTTTTCCTGGGCCTGGCGGCGACTACAGCGGTGCTGTTCTGTCCTGTCGCGGGCTGC
GGTGACTCGCTGGCGGGTCTTC
>‘000203a-057.scf” came from CONTIG 48 at offset 0;
“E:\SEQUENCE\export\EST_db\000203a\000203a-057.scf’ (10>722)
CTCTATACTATGGATCCCCGGGCTGCAGNAATTCGGCACGAGGAGGTATGTCTGAATGTGTTTCGACTATTTACAGCCCCGTTTCCTCGCAGGGCCCCATGAG
TAAGCTGTGGCGGCGCGGGAGCACCTCTGGGGCTATGGAGGCCCCTGAGCCTGGGGAAGCCCTGGAGTTGAGTCTAGCGGGTGCCCACGGCCACGGAGTGCAC
AAGAAAAAGCACAAGAAACACATGAAGAAACACAAGATAAAACACTACCAGGATGAAGAGGCTGGGCCAACGCAGCAGTCTCCTGCCAAGCCCCAGCTCATAC
TCAAATCAAGTTGGGCGGGCAGGTCTTGGGCACCAAGAGTGTTCCTACCTTGACTGTGATCCCTGAGGTCCTCGCTCACCCCTCTCCCTTATGTNTGGGAACA
TGAAGAGTACCTGTGAAGGAGCCCCCTGACATACCGCGCTGCTGGAGAAACAAAACTGCCCCTCCCATGGGNACTGCTGNGGNTANAGCCAGAGAAGAGAATA
CGAGGGCTGAGCTTGNAAAGGGAGCGAGACAGAAACCAAAGAAACAGACGCTGCACGTGCAGAGTTGTGAAGCCGAGCACTCCGGTGCCTCTGCTGGCTGCGG
CCCTTACAAAAGGGGAGGGGGGGCGGAAGGGGGGGAGGGGCGGGAAAAAAAAAAACCCCAAGAGCGGGGGGGGGGGCGGGGGGGGGGGGGCCCCG
>‘000203a-058.scf” came from CONTIG 49 at offset 0;
“E:\SEQUENCE\export\EST_db\000203a\000203a-058.scf’ (38>620)
NAAAGACTTCATGAAGGAGAAGAAAAGGAAAAAAATCAAAGGATGACTACAGCCTCTTfCATCTCAGCTTTGCAAAGCATGACTCAACAGAGAGATCACATTC
TTGAAGACTCTCCAGACCACAGATTGAAGATAAGTGTTGTGCTGCACTATTTCTGTTGCTACTCGTGAGAGTCACTACCAGCCTCTCTTCTCAGGATTAATGA
ATGGGCCAGAAACCGGGGGCAGAATGACTNCAGGGGCACCCCCAACACCAGATATTGGCATAAGGAAACAGTACAATGAAAAAAGTCATACTTGGACCCTTGT
CATTCCACANAGCATGATGTACTATCTAAAACAAAAAGAAGAGCTGCTTTGGAGAAGGGTTTTAAAAGTFGTTTATCAAAANAAAGATTANAAGAGGAAAACT
CCAGTNTATNAGATGGCATTTACTTTNAAGGCTCTGTCCCTGTGTAGGCATGAGATTGATACAGNACAGACGGGCTGCCAANCAGNTACTAATGTNTCTGCTC
TAAGAAGGGNGCTGTTGCTTCCTTGTGATATCTTGCAGAGCCGACTGAGGACAGAAACACAATAAG
>‘000203a-059.scf” came from CONTIG 50 at offset 0;
“E:\SEQUENCE\export\EST_db\000203a\000203a-059.scf’ (39>620)
CAATTCGGCACGAGGGTGAGTGACATCGTCTTTAAACCCTGCGTGGCAATCCCTGACGCACCGCCGTGATGCCCAGGGAAGACAGGGCGACCTGGAAGTCCAA
CTACTTCCTTAAGATCATCCAACTTCTGGATGATTATCCAAAATGCTTCATTGTGGGAGCAGACAACGTGGGCTCCAAGCAGATGCAGCAGATCCGCATGTCC
CTCCGCGGGAAGGCTGTGGTGCTGATGGGCAAGAACACGATGATGCGCAAGGCCATCCGAGGGCATCTGGAAACAACCCGCTCTGGAGAACTGTGCCTCACAC
CGNGGAATGTGNNGCTCGGTCACCAAGAGACCTACTGAGACAGGACAGCTGCTGCCACAGGGCCACTGCGCCCGGCTGTGCATACGCCGGGAGACCTGGCCAC
CAGACATGNCTGGGCCGAAAGACTTCTCTCAGCTTAGCACACACGATACTCAGGCAGAGAACCTGAGAGGCATGATAAAAGAACAGAGCGCAGAGCACGCGAC
ACGACACCCCTTCCTGCGCCCCAAGGGGAAAGCAGATACCCAGCTAAAAAGACCGCTCCTCGGGGGG
>‘000203a-083.scf” came from CONTIG 50 at offset 0;
“E:\SEQUENCE\export\EST_db\000203a\000203a-083.scf’ (45>614)
GGACGAGGGTGACATCGTCTTTAAACCCTGCGTGGCAATCCCTGACGCACCGCCGTGATGCCCAGGGAAGACAGGGCGACCTGGAAGTCCAACTACTTCCTTA
AGATCATCCAACTTCTGGATGATTATCCAAAATGCTTCATTGTGGGAGCAGACAACGTGGGCTCCAAGCAGATGCAGCAGATCCGCATGTCCCTCCGCGGGAA
GGCTGTGGTGCTGATGGGCAAGAACACGATGATGCGCAAGGCCATCCGAGGGCATCTGGAAAACAACCCGGCTCTGGAGAAACTGTTGCCTCACATCCGGGGG
AATGTTGGCTTCGTGTTCACCAAGGAGACCTCACTGAGATCAGGGACATGCTGCTGGCCAACAGTGCCAGCTGCGCCCGGCTGTGCCATACGCGGGTGAAGCC
TGGCCAGCCAAACATGTCTGGGCCCGAGAGACCTCTCTTCAGCTTAGCACACAGAANATTCAGGCACATGAAATCTGAGAGGCACGATAGAAGAACAAGAGCG
CACGAGCAGCGTGACAGCGACACCCCTTTCTGGCGNATGACAGGTGACAGCAATT
>‘000203a-060.scf” came from CONTIG 51 at offset 0;
“E:\SEQUENCE\export\EST_db\000203a\000203a-060.scf’ (1>262)
GGGCTAACAGTCCGCGAGCCCGGCAATCCGCAGCCGGGCCCACAGGAACATGCGTCTGCTTGGGGGGGAGAGGGCCGGGCTAGAGCGAGCAAGGTGAGGGGGG
GGGGGGGGGACCTCCCGCGGATACAAGGTCACACACCCCTCCTAATGCAGAAGGCGACGGTTGCAGGAAGGGCAAAATAAGGACTCGCAAGGTGTCTAGGGGA
ACGAGTAAATGAAAGGCCACGGCGCGAGACGCGAGCGACCACCCAGGAGAACCGCG
>‘000203a-061.scf” came from CONTIG 52 at offset 0;
“E:\SEQUENCE\export\EST_db\000203a\000203a-061.scf’ (29>494)
NAATTGGGCACGAGGGTGAGTTTCATTGAGTTAAATAAATACCTTTTGGAAAGGAGTTTGCCGATGCACCAAAAAAGCCTGTCTGCGCTGTAGGAATGTGTGG
TGAAGCTCAATTTCTGTTTTATGAAACCTGTTTGGGCGGGGGTCTGGGGGTTGCACAGAGAATGAGTTCTTGTATTTCGCGTCACACAGGTAGTTATGGAAAT
ATGTTATTGTACTGTGTAAAGATGCCCAGCCATTTTGATTGTTTGGCTTTTTACTTGTACCTTTTCAAGCTTTTGCTATACATCTGGAACCCTCAACACATAC
TGTGTTGTACTTCCTTTTGTAATGATTTTTAATGGAAGTTTGCACATAACTCTTTGTTATACTGTAGGATAATCTTGGGGGAAAATATTTTGCATATCAAAAA
AAAAAAAAAAAAAAAACCGAGGGGGCCCGCCCCCATTCCCCTTFA
>‘000203a-062.scf” came from CONTIG 53 at offset 0;
“E:\SEQUENCE\export\EST_db\000203a\000203a-062.scf’ (1>630)
CGGGCGCCGTTAACTAGGTCCCCGGCTCAGCAGACACAGTGTCGTGAAAACCACCGTTAAACCTAAGCCAAAATGGGAAAGGAGAAGACCCACATCAACATCG
TGTCATTGGGCACGTAGATTCAGGGAAGTCTACCACGACTGGCCATCTGATCTACAAATGTGGCGGGATCGACAAGAGAACAATTGAAAAGTTCGAGAAGGAG
GCTGCCGAGATGGGAAAGGGCTCCTTCNAATATGCGTGGGTCTTGGACANACTTAAAGCTGAACGNGAGCGNGGNATCACCATTGATATCTCCCTGTGGAAAT
TTGAGACCAGCAAGTACTATGNTACCATCATTGATGCCCCAGGACACAGAGACTTCATCAAAAACATGATTACAGGCACATCCCCAGCTGACTGTGCTGTCCT
GGTCGGTGCTGCTGGGTTGGNNGAATTGAAGCCGGATCTCCAAGACGGCAGACCCGNGAGCTGCTTTTTGCTTACACCTGGNNGNGAAAACACTATTGTGCGN
NTACAAAGGATNCACTGACACCTATACAGAGAATCAANAATGTTAGNANCACACTTATANAAATGCTCACCCGACANACATTGGCCATTTGCTGAAGGACAAG
CTAACAAGCT
>‘000203a-075.scf” came from CONTIG 53 at offset 27;
“E:\SEQUENCE\export\EST_db\000203a\000203a-075.scf’ (41>615)
CGGGACACAGGTGTCGTGAAAACCACCGTTAAACCTAAGCCAAAATGGGAAAGGAGAAGACCCACATCAACATCGTTGTCATTGGGCACGTAGATTCAGGGAA
GTCTACCACGACTGGCCATCTGATCTATAAATGTGGCGGGATCGACAAGAGAACAATTGAAAAGTTCGAGAAGGAGGCTGCCGAGATGGGAAAGGGCTCCTTC
AAATATGCCTGGGGTCTTGGACAAACTTAAAGCTGAACGTGAGCGTGGTATCACCATTGATATCNTCCTGTGGNNAATTGAGACCAGCAGTACTATGNTACCA
TCATTGATGCCCGCAGACACAGAGACTTCATCANAAACATGATTACAGGCACATCCCAGCTGACTGTGCTGTCCTGATCGTGTGCTGTGNTGGNNGAATTGAG
CCNGCATCTCCAAGACGGCAGACCCGGAGCTGCCCTTTTGGCTACACCTGGTGTGAAAACACTATGTTGGCGTTACAATGGATNCACTGACACCTTANCAGAG
AATCAANAAATGTAGAAGCAGACTATTAAAAATGCTCACCCGACAGACTTGGCCATTTGT
>‘000203a-063.scf” came from CONTIG 54 at offset 0;
“E:\SEQUENCE\export\EST_db\000203a\000203a-063.scf’ (10>605)
GCTCTATACTATGGATCCCCGGGCTGCAGCGTCACTTACGTCACTCGTTCGGAGTCGTATATCGGGGGAAATTGCTACATTCTGTCAGGGTCACGTGATGCAA
CCCTTCTGCTCTGGTACTGGAATGGAAAAAGCAGTGGTATTGGAGATAACCCGGGCAGTGAGACTGCCACTCCGCGGGCCATTCTGACAGGCCACGACTACGA
GATCACTTGTGCTGCTGTCTGCGCGGAGCTCGGCCTCGTGCTAAGTGGCTCCAAAGAGGGACCATGTCTCATACATTCCATGAATGGNAGACTGNNTAGGGAC
TTGNAGGNTCCANAAAACTGCGTGAAACCAAANCTCATTCANGCGTCGAGAGAGGCCATTGTGTCATTTTTATGAAAATGGGCTCTCTGCACATCATGTAACG
GAAAGCTGAGCCACATGGAACGACATACATAAGGCATGACTGACGGNATGGCAGACTGCTCACGGAGAACAGGGGGCTCAGTCTGCGGGTCGACTAACATGTC
GCTACAGTGGAGCGGATCGGCTGGCTGCTAACAAGGCGCTGTGCTCCTAGACACGTGTTCACATCACGGGAGCAACACCCT
>‘000203a-064.scf” came from CONTIG 55 at offset 0;
“E:\SEQUENCE\export\EST_db\000203a\000203a-064.scf’ (10>605)
GCACGAGGCCTGGACCCCCTGGTCCCCCAGGTCCTCCCAGCGGCGGCTACGACTTGAGCTTCCTGCCCCAGCGACCTCAAGAGAAGGCTCACGATGGTGGCCG
CTACTACCGGGCTGATGATGCCAATGTGGTCCGTGACCGTGACCTCGAGGTGGACACCACCCTCAAGAGCCTGAGCCAGCAGATCGAGAACATCCGGAGCCCT
GAAGGCAGCCGCAAGAACCCCGCCCGCACCTGCCGTGACCTCAAGAGTGCCACTCTGACTGGAAGATGCGAGATACTGGATTGACCCNCACCANNGCTGCACC
TGGATGCCATTAANNGTCTCTGCACATGGAACCGGTGAGACCTGGTATACCCACTCAGCCANGTGGCCCATATAACTGTATATCACAGAACCCAGTAAAAAGC
AGGTCTGTACGGGAGACTGACGGCGATTCAGTCGATTGCGGCAGGGTGGACTGCGAGGGCATCATGATTCTGGCTGAGNCACGAGCTCAAAATACTACATGAG
ACAGGNCTATGACACAATGCACTAAAGCCGTCTCAGCTCAGATGA
>‘000203a-066.scf” came from CONTIG 56 at offset 0;
“E:\SEQUENCE\export\EST_db\000203a\000203a-066.scf’ (39>329)
GTCTCAATGTCCGTGGCGCTGAGGCAAGCGTTGTGGGGGAGAAGGGTAGCGACTGTAGCTGCCGTTTCCGTTTCCAAGGTFLTCGACCAGGTCGTTGAGCACT
FFCCACATGGAGGCTGGCACAGGACCAAACTCGAGACACGCAACTCATAACAGTTGATGAAAAATTGGATATTACTACTATAACTGGTGTTCCAGAAGAGCAT
ATCAAAACTAGAAAAGCCAGATCTTTGGTCCTGCTCGNCATAGATGCAGTCTGTAGTTAACAAACACAAGAATGGAGATGGAGGTTG
>‘000203a-067.scf” came from CONTIG 57 at offset 0;
“E:\SEQUENCE\export\EST_db\000203a\000203a-067.scf’ (45>669)
GCACGAGTGGCGGATGACGCCGGTGCTGCGGGAGGGCCCGGAGGCCCCGGGGGCCCCGGAATGGGAGGCCGCGGTGGCTTCCGCGGAGGCTTCGGTAGTGGCG
CCCGGGGCCGGGGTCGCGGCCGGGGTCGGGGCCGGGGCAGAAGCCGCGGAGCTCGCGGAGGGCAAGGTCGAGGACAAGGAGTGGCTCCCCGTTACCAAGCTGG
GCCGCCTGGTCAAGACATGAAGATCAAGTCTTTTGAGGAGATCTACCTTTCTCTGTGCCTATCAAGAGGCTGAGATATTGACTTTTTCCTGGGAGCATCCTTG
AAGATGAGTTTTGAAGATTATGCCGGGCAAAAGCAGACCCGGGCTGCCCAGGAACCAGTTCAAGGCGTTGTTGCTTTGGGGGATAGAACGACTGGGGGCTGGT
GGCAGGCCCAAGAAAATACCCTGCCTCCGGGGGCCATCTTCTGCTAAGTGTCCACGCCCGGGCAAGAGCTTAGGGGAACANAAGACACCCCCCCGTCTGCAGG
GACGGCTGGGTCCGGCGGGCCCTACCTCCCAAGACGGCTCCTCGCCCGGCCAAACGGAGAGCGCTGACAGTCCTTGCCGGCGGTGCCCTGGCATCCAGCCTTG
TCTGCA
>‘000203a-068.scf” came from CONTIG 58 at offset 0;
“E:\SEQUENCE\export\EST_db\000203a\000203a068.scf’ (40>680)
TTTTTTTATGGCTTCTTCTTTCTTTATTGGACGCTTTGTAGATGTCACGCAGGTCTAAAAGTTACACCGTTAAATAATTATTTAAAAACCAACCAGGATTAAG
GCCCTGGCCCAGAGCTCCAAACCAGAAGCAGAAAGGAATGGTGGCGGTGGGCTGGGGGGGTATTCCTCCAACATCACCAAAACCCAGAGAACGAGGATCCTAA
GCTTTTCACAGGCCAACCCGGGCACGGGCCTGCAGGCTGACCCTCGGAGGCCTCTGGCTGCATCACTATCAGATCAAAACCAGCGAGGAGCTGCCGGGAACAG
CCAGCCGAGTCCAGACATGGACACAGTAGCTGGATGGACACGAGACGGACAGGTCCTGTCCAGCTGTGGACAGGATTCAGATGCAAGCTAGGCAGTGGGGGCA
GGGGCTGGGGAGCAGAATGAAGCATGCAGGAGGGGCCCCGGGGCCTGGCTCANCCACCGGCCGCCGAGCCTCACCGTGTGGGNTCGCTGGGGNCTGGGCTCCC
GCCGACACTGGACTTGAGGCTCTGNAGCGAGAGTTCCAGCCATTGTGATGTTGCTCTCACAACAAATNCTTGCCACTGGGAGAATTGAGATGGTGACAAACTA
CGCCCAAGCATGTGGATGCCAGC
>‘000203a-069.scf” came from CONTIG 59 at offset 0;
“E:\SEQUENCE\export\EST_db\000203a\000203a-069.scf’ (12>643)
CTCTATACTAGGGATCCCCGGGCTGCAGTAATTCGGCACGAGGCTCGGTTTTTTTTTTTTTTTTTTGCTCTAATTAAAATTTTTATTGAAATCTCTCAAACGT
TACCAAGAAATAGTTTTTGCAAAAGGGAGGGAAGGGAAAAAACAAACAACAACAACAAAAAACAGCTAACAAGCAAATTCAACATGGGAGCTCCCTCTGCTGG
TCTGCAGTAGGTTGATATGTTACAAACACATTCCCAGAGACAAATCTATTTGCTGGAGAAGGGACAAAAAACAGGTGTGTGGGCTTTGCCTCNAGAGAGAACA
CTGGCATGCAGNGAGCGGGGTAGTGAAAGCAGAGGAGCAGCGCAGCGCTAAGTCGCTGGTACAGAAGTACGGGCCACACTCAGGCTATGNGTAAAGGCAGCCT
GTGACTCTATGTCTCTGCATGACTGAGACAGGTGGCAAGNAACTGGGTTGGAGCCTACTATTGTCTTGCTCTCGGGTTGCTACAACCTATGTAGGTTGCGATT
AGACGACGAGCCTCAAGATTTGCGCTTTGTTGACATGCAATGCAACTAGCTGTATTACTTAAAACTTTACCTATGTGAAAAATAATCCGTGATCAAGGAAGGC
AAAAAAACTCTTT
>‘000203a-070.scf” came from CONTIG 60 at offset 0;
“E:\SEQUENCE\export\EST_db\000203a\000203a-070.scf’ (19>728)
CTACGGCATCCCGCGGGCTGCAGGTCGAGTTTTTTTTTTTTTTTTTTTTAGTTAATCTTTACATTTAGTGCTTTAAAATAAATATAATAATATAATTATCAAA
CATACAGTGAGAAGTAAAGCACACGTGTGAACGGCATGTCACAGGAGTTCACTCAGGACTGTTTCAACACTCAGCACTGGAGAAACCGCACAGGCCTACCTAT
GTACAGACGACCCAACCGCCAGGGCGAGGCCACCGCGTCCCCTCCGTCCTCATGGACACGGCCACTCCCCCTTGCGTTGAAATGACCAGTTTGCATGTTTTAA
CTTTTCTCTCCGCTTCGAGCTTCAGTTTTTTTTTTTTCTTTTGCAGTTTTGAAAAAATTCAAGTAACACTCCCAAGAAAAAAAAAGTGCAAACTAATAAGGGA
CTCAGAGTCCGGCGCCGGTCAGGGGCAGCGCACAGCGGGGGGGCAGCCGGCGGAGTCTGTCCCGGAACACGGGGCGCAGGACCCCGCCCACTCGAGGAGGGGG
GGACGGAGGCCGGCCTGGGTCCAAAGCGCCAGACCTTTGTTGAAAGCAGCACAGCCCGCGAACGAACGCAGTCCGCGGCCGACTTCGGACCAAGGAAGGAGAG
GGGGAAAAATAAAAGTATTAGGATCCTTTTATAAAATATAAAATTTTCAAATTTTATAAAGGGCGCCCCCGGGGGGGGGGGCCCC
>‘000203a-071.scf” came from CONTIG 61 at offset 0;
“E:\SEQUENCE\export\EST_db\000203a\000203a-071.scf’ (38>713)
TTGACATCCTCCATTTCGCTGTGCTGAGTTAGTACAGGGAAGACGGGGAAGTGAAATGCCAACAGTAGTAGATGGGGGACTTCCCTGGTAGTCCAGTTGTTGA
GACTTCGCCTTCCAGTGCTGGGGGGTGCAGGTTCAGTCCCTGATGTGGGAGCTAAGATCCCTCATACCTCATGGCCAAAAAACCAGAATGTAAACAACAGATA
CAATATTGTAACATATTCAATAAAGAATTAAAAAAAAATTTTTTTATAAAATATACTTAAAAAAAAACCTGACAGTTTCACAGAGAGGGCTGTGATAGGATTG
CTCATGAGGAATCCAAGGAATAGAAGTTTTGATAGAGGATGATGGGAAGTGTGTTAGCAGAAGTAGGACTGCCTACTGTCCATCTGGACCATGNAAAGCACAN
ATTCATCCCGCACAGCTTGGAGAGAATGTCTCACTAAGAGCTCATGCCTTGTATTCCTCCCACATTATTTGTATTGTTGGTTTTATATACGTTTTTTTGCTGA
TATTACCATATTAGGTTTGTGTGATGATTGTAGGACATTCTATGTAGAGAAAGATAAACTTAAAAAAGAAGCCCTTTTTCTTTTTTTNTTAAATAATGCATCT
TAAGTGAGTCACCCTTTCGGCAAAAAAGAGAACACTTTACTTATTACTATTTCTT
>‘000203a-072.scf” came from CONTIG 62 at offset 0;
“E:\SEQUENCE\export\EST_db\000203a\000203a-072.scf’ (38>334)
CTGGTGCTTTCGGGCTCGCTGCGGTGCCGACAGTCCGTTCATGCGTCGCGTTTGAGGGCAGGGGGTGGCTCAGCGGCTGGCTCGCAGCTTTCTCCGCTGGCTG
AGGCCCGCCACAGCCGACATGGGCTGTTTCTGCGCGGTTCCGGAAGAATTCTATTGCGAAGATTTGCTTCTGAATGAATCGAAGTTAACTCTCACCACCCANC
AAACAGGCATCAGAAAATCACGAAAGGGCTCATTGTCCTFFGAGCACCGTATCCGCCACTCAACCCTGGGGAGGTGAGTATTTTTGGCTTGC
>‘000203a-073.scf” came from CONTIG 63 at offset 0;
“E:\SEQUENCE\export\EST_db\000203a\000203a-073.scf’ (1>639)
CGGGGCGCCCTCTAAACTATGGATCCCCGGGCTGCAGGCCCAGTTCCCTCCCAGAACATTCTTGGGACCAGCCACCTTTCCCCAGGTGTGTGCTGCCCACTGC
CACCCAGAGGTGGGATGGCAGGTTCCAGGTTCCCTCTTGGATCCCAGGCTTCCCCTGACATCAGCACCATTCAGTGGTTTTCCTGGCTCCATCGCTATCGCTT
CATGCTGAATGGACAGGACTGTTGACCTGTCTCAAGAAGCCCATAAGATTTGAGCAGAAACGNTGACTTCCTTGTACCTCTGCCAGAGCAGCTCCCTTCTCCC
CCCGAGAATTTCACCAGATCTCTTCATGCTGCTCTTNATTTNCGTACTTGAAGTGGNTCTGCCGACAAACAGCAGACTTTGTGTGTCCCACTCTGACTTGCAG
GACGGNAGNTTCTTTCTCTTAAGTTGATCGCTTCTTGCTGTGACTGNCCCGCTGGNGAAAATGGTTTTGCCTCGTTTTAAGTGAACAGGAGACTAGATGCTGT
ACTAAACAGATGGAACCGAGAGAAACACTACCATCAGTAAAGACCACCACCCAACACTCATAAACGCTAACAAATCTGCTGAAAGATGTAGCTGGAAAAAAAA
AACGGGGGCCGGCCACCTTA
>‘000203a-074.scf” came from CONTIG 64 at offset 0;
“E:\SEQUENCE\export\EST_db\000203a\000203a-074.scf’ (1>580)
GCGGGCGCGCTCTAAACATGGATCCCCGGGCTGCAGGTTCCTGTGTCGTCTTGGAGGTGACTCGGCGTGATTGAATTTGCGGCATCTTCGCATTCACTCACAG
GTCAAAATGCAGATCTTCGTGAAAACCCTGACCGGCAAGACCATCACGCTGGAGGTGGAGCCCAGTGACACCATCGAGAACGTGAAGGCCAAGAATCCAGATA
AGGAAGGCATTCCCCCTGACCAGCAGAGGCTCATCTTTGCCGGCAAGCAGCTGGAAGATGGCCGCACTCTNTCTGATTACAACATNCAAAAGAGTCGACCCTG
CACCTGGNCCTNCGTCTGAGGGGGGNATGCANAATTTCGGAAACCCNNTGACGCAGACATCACCTGGAAGGGAGCCCANGACACCACGANAACGGAAGCCNAA
ATCAGATAGAGGCATNNCCCCGACACAAGCTCATCTTGCGCAGCACTGGAGAGGCGCCTCTTTGATACACANCAAAAGGCGACTGCCCGGCCTCGGGAGGGGG
AGCAATCTCGAAACCGACGCAGACATACTGAGGGGGCAGCACACCAAAGAAGCAAAGAAAAAAGA
>‘000203a-076.scf” came from CONTIG 65 at offset 0;
“E:\SEQUENCE\export\EST_db\000203a\000203a-076.scf’ (45>598)
GCACGAGGCCTTCATCCAGCACCTTCCCCTGAGTGAGCGCATCCGGGGCACCGTCGGACCAAAGAGCAAGGCAGAGTGTGAGATTCTAATGATGGTGGGCCTG
CCCGCTGCTGGCAAAACCACGTGGGCCATCAAACATGCAGCCTCCAACCCCTCCAAGAAGTACAACATCCTGNGTACCAATGCCATCATGGATAAGATGCGGG
TAATGGGCCTACGCCGTCAGCGAAACTACGCCGGCCGCTGGGACGTCCTGATCCCAGCAGCCACTCAGTGCCTCAACCGTCTCATCCAGATTGCTGCCCGCAA
GAAGCGCAACTATATCCTANATCAGACAAATGTTTATGGGTCAGCCCAGAGACGAAAAATGAGACCATTTGAAGGCTTTCAGCGCAAAGTATTGTATTGTNCC
NACTGATGAGACTGNAAGACGAACAATAAGCGACTGACGAGAAGGAAGGAGTCCNANACACGCGTCTTAAAATGAAAGCACTTCACGTGCCGATGTGGGACTT
GTGGAGAGNGCTGTCATGAGCTGCAGGAAAGGAGCGAC
>‘000203a-077.scf” came from CONTIG 66 at offset 0;
“E:\SEQUENCE\export\EST_db\000203a\000203a-077.scf’ (46>306)
CACGAGGGGAATCTTGTCCTTCCAGGTCCGCCAGTTTAAGCGCCTTTATGAACATATTAAAAATGACAAGTACCTTGTGGGCCAGCGCCTCGTGAACTATGAA
CGGAAATCCGGCAAACAAGGCACATCACCACCACCTCGACAGTCGTCCCAAGAATAAAGTGGTTGTCTCCACTACCTTGGCCTTCGGCTTGCCTTCACGTGTC
CTTTTTTGTGGACTTCTCTCTCTGGAGATTTCCCCAGTGATCTCTCAGCGTTGTT
>‘000203a-078.scf” came from CONTIG 67 at offset 0;
“E:\SEQUENCE\export\EST_db\000203a\000203a-078.scf’ (39>281)
CTAGTCTGAGTTTTTTTTTTTTTTTTTTTTTTTTTTTTTCCTGTACTACTATCACTATTCTCAGGTGGGTTTTTGAGAATGAATGTGCAGAGTTTATGATGTG
TGTCAAGCATGCCTCGATAGCCACAGGCTTTACAAAAATTACCTATTGCTTTGGATTGACATGCAAATCTGTTTCATGATCTCACACTCACGACAGAAAACAA
ATTTTTTTATGAATCCATCCACCATGCTT
>‘000203a-079.scf” came from CONTIG 68 at offset 0;
“E:\SEQUENCE\export\EST_db\000203a\000203a-079.scf’ (16>24)
ACAGGGATC
>‘000203a-080.scf” came from CONTIG 69 at offset 0;
“E:\SEQUENCE\export\EST_db\000203a\000203a-080.scf’ (38>623)
NAATTCGGCACGAGGCAAGCGCCTGCTGGAGCCCCCGTGCTCCTTGCACTTGAACTCTATGGGGTTTGGTGGGCAGAGGCTCAGGAGTCCCCTGGATTTCCCC
AGCTGGTATCCTGGGACGTGGTAAGCCCTGGGGCTGGGGTAGCATGGGATCCCCCGAGGACCCANATTCTGGTACTNAGGGCAAGGNGAGGNGAACCCGNACC
TCANCCGTCCCCCAGTCTACAGCCTGAGCCCAGTGTGCTCCCAGCTCCCCANTCCNCATGAAGCCTGCCGGNGGCTGGCAGNAGGGNTTAGAGGNNCTGGCCT
TCGATTCCTTTTCTGTCGCGCTGCTTTCACCCGCTTGCTGCAGCTTTGCTCTGGCCTGATGATCGTGCTTTGTTCTCTGTACTGTTAACTGAGCATGCCACAT
TTGTTGAAATGTTGTTCAAGTGTAAGCAAGGAGAGGTCCAATTGTGATGGGGATGGAGGCATGGACTCTGCTTCTATCCTTCTACTTATCTGAAATGTTGCTT
CTGCTGTTGGATTATTATACAGGGCAACCTATACAGCGAAAAAAAAAGGCAAAAAATTCTCTACCACGAGA
>‘000203a-080.scf” came from CONTIG 70 at offset 0;
“E:\SEQUENCE\export\EST_db\000203a\000203a-081.scf’ (41>563)
CTCCAGTTACCTCTGCCAGTACCGCTGTGTCAACGAGCCGGGCCGCTTCTCCTGCCACTGTCCACAGGGCTATCAGCTGCTGGCCACGCGCCTGTGCCAAGAC
ATTGACGAGTGTGAGTCGGGTGCGCACCAGTGCTCTGAGGCCCAGACTTGTGTCAACTTCCACGGNGGCTACCGCTGTGTGGACACCAACCGCTGTGTGGAGC
CTTACGTCCNAGTGTCCGACAATCGCTGTCTCTGTCCGGCCTCAACCCCCTGTGCCGGGAGCAGCCCTCATCATCGTGCACCGTATATGAGCATCACCTCGAG
CGGAGCGTACCGCGGACGTNGTTNCAATCAANCANCNNTCGTCTACCTGTGCTACATGCTTTCAATCGTGCTGTAACTCGCAGGAACTCTACATAGCAATCAC
ATGCACGCTGCTGTCTCGCTCGGCTGGACGGCCCCGGATACGGCTGACTGAGAGTCACTTACTCTCTGATACGGCACTTTTGAATACGCTTGGGGCTACTTTG
GGGGGG
>‘000203a-082.scf” came from CONTIG 69 at offset 0;
“E:\SEQUENCE\export\EST_db\000203a\000203a-082.scf’ (48>533)
GCACGAGGGCCTGCTGCAGCCCGGCTGCCAGCTGGAGTCCCTGTGGGTGAAGTCCTGCGGGTTTACGGCCGCCTGCTGCCAGCACTTCAGCTCTATGCTGACC
CAGAACAAGCATCTCTTGGAGCTGCAGCTGAGCAGCAACCCGCTGGGCGACGCGGGCGTCCACGTGCTGTGCCAGGCCCTGGGGCCAGCCGGCACTGTGCTGC
GGGTGCTCTGGGTGGGCGACTGTGAGCTGACGAACAGCAGCTGTGGCGGCCTGGCCCTCACTCTGCTGGCCCAGCCCCACCTGCGGNAGCTGGACCTGANNCA
TACGGNCTGGGCGACCCCCGCGTCTGCAGCTGCTGGGGCAGCTGGAGCACCCGCTGCAGCTGGAGCACTGTCCTGTCGACTCTATGGACCGAGCATGGACGAC
GCTGCGGCTGTGGAGAAAGCAGCTGGNCTGCGATCTTTCTGACCCGTCCCCAGNGCGTNATGAAAAGTNCATCA
>‘000203a-084.scf” came from CONTIG 72 at offset 0;
“E:\SEQUENCE\export\EST_db\000203a\000203a-084.scf’ (47>388)
GCACGAGGCACAGTAGCATCACTTCAGAAAGGAGCCAGACTTATTCTCAAAGAACTATGTTCACACTTTTCAGCAGAAATAGCGATGGTTGTAACATATGTAT
CCCCTCCCTCGGATTGAAGGCACAATCTACAGTGTTTCTTCGCTTCTTTTCTGATCTGGGGCATGAAAAACCAAGATTGAGATTTGAACTATGAGTCTCCTGC
ATGGCAACATAATGTGTGTCACCGTCAGGCCAAACAGCCAGCCCTGAACGGTGGNTTTATTACTTGTGTATTTGTGTTGGATGATAAACACTCATCATCTCTC
CTGTAGTCCCTGCTCATTTCACTTAACCCTAN
>‘000203a-085.scf” came from CONTIG 73 at offset 0;
“E:\SEQUENCE\export\EST_db\000203a\000203a-085.scf’ (9>658)
CGCTCTATACTAGGGATCCCCGGGCTGCAGAATTGGCACGAGGGGAGCTCCGCATCCACACCGGCCAGCCCAGATCCCGAGGTCTGACAGCGCCCGGCCCAGA
TCCACAAGCCTGCCAGGAGCCAGCCGAGAGCCAGCCGGCCGCGCGCTCCTACCCCAGCAGTCTCTGTCCTTCGGCCTGAGCCCCGCGTCCTTCCCGGGACCTC
TGCCCCTCGGGCAGTGCTGCCACCCTGCCGGCCATGGAGACCCCGTCCCAGCGGCGCGCCACCCGCAGCGGNGCGCAGGCCAGCTCCACCCCGCTGCCACCCA
CCCGCATCACCCGGCTGCAGGAGAAGAAGACCTACAGGAGCTCAATGACCGNCTGGCTGTCTACATCGACCGTGTGCGGGCGCTGGAAACGAAATGCAGTCTG
CGCCTCGCACACTGATCTGAGAGGGGGCAGCCGGAGGGTCTGGCTTAAGCCCGCTCCAGCCGACTGGGGAGCCGCCAGACCTGACCGTGGNCAGACGCGCCGC
GCGCGGACGACAAGGGAAGAGTCAGGACCAGCACGCATCAAAGAGGAACGAGGCCCAGCCGCTAGACGAGGCGTCACCAGAGCGGCGGCTGTTAGAAGCCGGG
GGACGGACGGGGAGGCATGGGCCCGGGGCAAC
>‘000203a-086.scf” came from CONTIG 74 at offset 0;
“E:\SEQUENCE\export\EST_db\000203a\000203a-086.scf’ (48>633)
GCACGAGGATGAATTTTCACTGGCCCTTCGGCATCTGGTTGTGCAAAGCCAATTCCTTCATTGCCCAGTTGAACATGTTTGCCAGTGTCTTCTTCCTGATGGT
GATAAGCCTGGACCGCTATATCTACTPGATCCACCCGGTCTTATCTCATCGGTACCGTACCCTCAGGAACTCTCTGATTGTTATTATAGTTGTTTGGCTTTTG
GCTTCACTAATGGGTGGGCCAGCTCTGTACTTCCGGGACACTCTGGAGTTGAATAACCACACTCTTTGCTATAACAACTTCCATGAGCATGATGTGGACCTCA
GGTTGNTGAGGCATCATGTTCTGACCTGGGAGAAAGTTATTGTTGGGTACCCTCTCCCTCTGCTAACAAGAGCATTTGCTACTTGGCCTCATCTCAAGAGAAG
AACGAGCACCTGTACTCAGAAGCCTCCTGACCACCCGGCGNGGNCATGCCTTNCGATTGCTGAATCCTATCACTGTTACATTGGAACCACGACCACACATACT
ATTCACCAAGCTACAGCACACCCCTTCACGCCGNGTTCTCAAATGCTGACCCCCCTTACCCGATATAAAAG
>‘000203a-087.scf” came from CONTIG 75 at offset 0;
“E:\SEQUENCE\export\EST_db\000203a\000203a-087.scf’ (46>645)
GCACGAGGATTTAATATTGTGGAGGGTGGGGCTTCCAGGTGTTACAGnGCTGGTTGCTGAGCCATGCCCAACTCTTTGCAACCCCATGGACTGCAGACCGCCA
GGCTCCTCTGTCCATGGTTGTCCAGGCAAGAATACTAGAGTGTGTTGCCACTCTCTTCTCCAGGGTATCTTCCGAATATAGGGATCAAACCTGGATCCCCTGG
ATTGCAGGCAGATTCTTATCCTCTGAGCCACCAGGGAAGCTCCTAGTCACCCTAAAACCTCCAAATTCTTAAAAAAATTACCCTATCTACTTCCACCCCAGTC
TTTCTCTCTTCTTCTTTTGGTGTCTTGSATTTTTGCTTTGGCTCTGCCACTGCATCACATCACCTCTTCCAGCCTGACTATGAGTCGCCTCAGACTCAGAGCA
GTTCACTCACGAATCTTGGCTTGACCACATACTCTCGNACTTGGCTCTGACTGCTTTTTTTATTGTTATTCGACATCTCCACCCGCGAGATCTCTTTGGACAG
CCTTGTATAACATCTGTTATACCTTTTGTACGCTATTTGGGAAAAATAATTAAAAGGGGCTCCCCCAAAAAATTACGCAA
>‘000203a-088.scf” came from CONTIG 76 at offset 0;
“E:\SEQUENCE\export\EST_db\000203a\000203a-088.scf’ (19>21)
TAT
>‘000203a-089.scf” came from CONTIG 77 at offset 0;
“E:\SEQUENCE\export\EST_db\000203a\000203a-089.scf’ (1>428)
AGGTGGCGGCGCTCTTATTATGGATCCCCCGGGCTGCAGAATTCGCACGAGGGAGGCCTTTCGGCCGCAGCCATGGCGCCCAGCCGGAATGGCATGATCCTGA
AGCCCCACTTCCACAAGGACTGGCAGCGGCGCGTGGCCACGTGGTTCAACCAGCCGGCTCGCAAGATCCGTAGACGCAAGGCCCGGCAGGCCAAGGCGCGCCG
CATTGCCCCACGCCCCGCGTCCGGTCCTCTCCGGCCGGTGGTGAGATGCCCGACGGGTCAGTACCACACGAAGGTTCGTGCCGGCAGGGGCTTCAGCCTGGAG
GAGCTAAGGGTGGCCGGCATCCACAAGAAGGTGCCCGGACCATTGNNGATCTCGTGGAGCCGNAGCGCGGANCAAGTGCACGGAGTCCCTGCAGGCCACGTGC
AGCGCTCAAGGAGTAN
>‘000203a-090.scf” came from CONTIG 77 at offset 0;
“E:\SEQUENCE\export\EST_db\000203a\000203a-090.scf’ (42>591)
NAATTCGGCACGAGGGGAAGTGTATAATTTCCTGGCCACTGCAGGTGCCAAGTACGGCGTGGGCTTCTGGAGGCCTGGCTCTGGAATCATTCACCAGATCATT
CTGGAAAACTATGCGTACCCTGGGGTTCTTCTGATTGGCACTGATTCCCACACCCCTAATGGCGGTGGCCTGAGAGGCATCTGCATTGTAGTCGGAGGTGCTG
ATGCCGGGNACGTCATGACTGGGATCCCCTGGGAGTTGAAAGGGCCCCAGGTGATTGGGCGTGAAGCTGACAGGCTCCCTCTCTGGCTGGACCTCACCTAAGA
TGTGATCCTGAAGGTGCGGGTATCCTCACAGTGAAAGGTGGCACGGGCGCCATCGGGNAGTACCACGGGCCTGGAGTAACTGCATCTCTGCCCCGCATGCGAC
CTCTGCACATGGTGCAGAATCGGCCACACTTGTGTTCCCTACACACAGAGAANAATACTGACAGACGGCGGCAATATGCACCTGTGAGAGATACTGTACTGCT
TGCTGCCTTTACAATTATATTACCTAGGCGA
>‘000203a-091.scf” came from CONTIG 79 at offset 0;
“E:\SEQUENCE\export\EST_db\000203a\000203a-091.scf’ (41>338)
TAATTCGGCACGAGGCCCCTTTCATCACCAACCCTGGGTATGACACTGGAAACGGTATTCATCUCCCGGCACTTCTGGGCAGCAGCCCAGTCTTGGGCAACAA
ATGATCTTTGAGGAACATGGTTTTAGGCGAACCACACCGCCCACCACGGCCACCCNCGTAAGGCATAAGCCAAGACCGTATCCGCCGAATGTAAATGAGGAGA
TCCAAATTGTTCATGTCCCCAGAGGAGACGTAGACCATCATCTCTACCCTCACGTTGTGGGACTCAATGCAAATGCTTCTACAGGCCAAGA
>‘000203a-092.scf” came from CONTIG 80 at offset 0;
“E:\SEQUENCE\export\EST_db\000203a\000203a-092.scf’ (47>391)
GCACGAGGCAGCCCGAGGACAGCCAGCAGGACCTGCCTGGGGAGCGCCACGCCCTCCTGGAGGAAGAGAACCGGGTGTGGCACCTGGTGCGGCCCACGGACGA
GGTGGACGAAGGCAAGTCCAAGTGCGGCAGCGTGAAGGAGAAGGAGCGTACCAAGGCCATCACCGAGATCTACCTGACCCGCCTGCTGTCCGTCAAGGGCACG
CTGCAGCAGTTCGTGGACAACTTCTTTCANNAGCGTGCTGCGCCCGGGAAACGCGTGCCACCGGGCGTCAAGTACTTCTTCGATTTTNCTGNACGAGCAGCAG
AAAAGCATGACATTANAGATGNANGACACCNATTNC
>‘000203a-093.scf” came from CONTIG 81 at offset 0;
“E:\SEQUENCE\export\EST_db\000203a\000203a-093.scf’ (44>356)
CAAAAACCAGAAGTGACGGGAGGTGCTGCGCTCCCCTGCGTCGTGGCAAAGTCAGCTGGCCTCTTGTGTGTGCGTGTGTGCGTGTGAGGAGCCGAGTGTGGGT
GTGTGGCGGGCGTGGGAGCAGCTTTCTCACATAGTGCCTTATACACGCTCTAAAGAAACCAGTCTTACATGTTTAAGAACAAGCAGTGTTACATTTTCTACAC
TACCTTNCATFLTCAGTAGCTTTGATGACCAGTTTTGCAGTTCATGGAGGAAATCATGGNNGCGTCCCAAGGGGCTCCCCATGCCCGAGAGCCGACTGGTCNT
GTGACG
>‘000203a-094.scf” came from CONTIG 82 at offset 0;
“E:\SEQUENCE\export\EST_db\000203a\000203a-094.scf’ (40>373)
GGGTTTTTTTTTTTTTTTTTTTTGTAATAAATAAAAAGTTTATTAACAAGGAATGCACTTTTCCAGCCACAAGTGTCTTCAAAAATTAACAAAACAAAAAAAA
TATATATATGGCCATAGTTCACAGTTAAGCAGCCAAAAGCTGCTCCAATTATAGCCTTTAAACAACATGTGAGCATCCTCCCTTTCCCTCCCCTTCAGTAAGT
ATATTCACAGCTTCAAGTCCTCTGTCCGAAGCACTCTCCACAGAGAGAAGTTAAGAGTCAATGCACCTTTCTGGAAAATTGTCTGAAAAGCTTTANNAAACAG
TACGTCAAGGAAACTGCTTCGGNTC
>‘000203a-095.scf” came from CONTIG 83 at offset 0;
“E:\SEQUENCE\export\EST_db\000203a\000203a-095.scf’ (42>489)
CGACAGCCTAGAGGGCTPCGTGCTGTGTCACTCCATCGCTGGGGGAACAGGCTCTGGCCTGGGCTCCTAGCTCTTAGAACGGCTCAACGACAGGTACCCCAAG
AAGCTGGTGCAGACATAGTCAGTGTTTCCCAACCAGGATGAGATGAGCGATGTGGTGGTCCAGCCCTACAACTCACTGCTCACGCTCTAGAGGCTGACCCANA
ACGCCGACTGTGTGGTGGTGCTGGACAACACTGGCCTGAACCGGATCGCCACAGACCGCCTGCACATCCAGAATCCCTCATTCTCCCANATCAACCAGCTGGT
GTCCACCATCATGTCAGCCAGCACCACACCCTGCGCTACCCCGGCTACATGAAACACGACCTCATCGGCCTCATCGCCTCGCTTATTTCGACGCCACGCTNCA
CTTNCTCTGACTGTTTCACCCCCTCCACAGNACAGCG
>‘000203a-096.scf” came from CONTIG 88 at offset 0;
“E:\SEQUENCE\export\EST_db\000203a\000203a-096.scf’ (43>460)
CATCAGGCTCGAGGGCTCTGTTGTGCGGACTGCTCCCCCTGGACCCTCTGGTTTCTCTGGGCCCTCTGACCTCTTTGATCCTGCTGGTAAAGAAGGGCTTCGT
GGGCCTCGTGGGGACCAAGGTCCAGTTGGTCGAAGTGGAGAGACAGGTGCCTCTGGCCCTCCTGGCTTTGGTGAGAAGGGTCCCTCTGGAGAGCCTGGTACTG
CTGGGCCCTCTGGGACCCCAGCCCACAAGGCCTTTTTGTNGCTCCTGTTTTTCTGGGTCTGCCAGCTCTACAGTGAGCGCGACTACACGTGTCGTGATCTGTG
GAGGGGTTGACACCTCTTTTCCTCGTTACATATAAAAATGTAAACCTGCCTTAACTGGACATATGACCTGATACTCACTTTATATTTTTTCTGGCTTTCTTAA
CAAA
>‘000203a-001.scf” came from CONTIG 1 at offset 0;
“E:\SEQUENCE\export\EST_db\000203a\000203a-001.scf’ (45>465)
GCACGAGGCTGTTTTATATTCGCCCATTCCAGTCCATTTTAATTCTCTGATTCCTAATATGTTGATGTTCACTCTTGCCATCTCCTGTTTGACCACTTTCAAT
TTGCCTTGATTCATGGACCTAACATTCCAGGTTCCTGTGCAATATTGCTCTTTTTATCATCAAACTTTACTTCTATCACTAATTACATCCATAACTGGGTGGT
GTTTTTTGCTTTGTTTTCATTCTCTTCTTCTTTTTGGAGTATTTTTCCACTGATCTTCATTAACATATGGGGCACCTACCGACCTGGGGGGGTGATC7TTTTC
ATGTCTTTCTTTTTGCTTTTTATTCTGTTATGGGGTTTCAAGCAAGATATGAAGAGTTTGCTTTCCTTTTCCGGGACACGTTTGTCAGATCACACAGACTGCC
GCTGGGGTTG
>‘000203a-002.scf” came from CONTIG 2 at offset 0;
“E:\SEQUENCE\export\EST_db\000203a\000203a-002.scf’ (48>297)
GCACGAGGGATTCTTATACTTTCTGAGGGAGTTTAATGACCACTAGAGCTTGTCCTCATATTTTTTTCAGCTTAATACTGTATGTCTCGTAAGATGGGCCTTA
TTGCCTGTATTCTTTGATATGTGATTAAGCCTATAGCTTTCAGTGACCAAACATTTTACAGAGTAAAAAATGTTAGGAAGCAGAAAAAGAAAATCTGATTTAT
TCTATGTCTCATTTATCCAGCCCTGCACTTAGATAGAAGTGTGC
>‘000203a-003.scf” came from CONTIG 3 at offset 0;
“E:\SEQUENCE\export\EST_db\000203a\000203a-003.scf’ (40>551)
TTTTGACAGAGAACATTTTTCTCACATACACTTTCAGAGTCAAAGCTGTGGATGGGGGAGATCCCCCCAGATCTGCAAGAGCCACGGTCTCTCTCTTTGTGAT
GGATGAGAATGACAATGCTCCCACTGTCACCCTTCCCAGAAATATTTCCTACACTTTACTGCCACCTTCGAGTAACGTCAGGACAGTAGTAGCTACGGGGTTG
GCAACAGACAGTGATGATGGCATCAATGCAGACCTTAACTACAGCNATGGGGGAGGGATTCCTTCAAGCTGYTGAGATGATTCACCAGTGGGNGGGTTTTCYF
AAAGGAAACTCACCCAAAGCATTATGGCTTGCACAGGNTGGTGTGCCAGNGATGACAGGGGCAGCTTCCCATCTACACGATCTGTGCTGTGTTGTCATGAAAG
GTTCTAAGCACTGGATGACTCCCAAAGCAAAACTGCCNCCCCATCACCAGAATATACGGGACCAGCTATAATTACACAAAATAATTTGGGGGGGG
>‘000203a-004.scf” came from CONTIG 4 at offset 0;
“E:\SEQUENCE\export\EST_db\000203a\000203a-004.scf’ (43>365)
GCACGAGGGCCCTTTGACGTTCCGGCCGCGCGCCCCGCGCCTCGTCGCTATGCCTCGCAAAATTTAGGAAATCAAGGACTTTCTGCTCACAGCCCGCCGCAAG
GACGCCAAGTCCGTCAAGATCAAGAAAAATAAGGATAATGTGAAGTTTAAAGTTGGATGCAGCAGATACCTTTACACCTTGGTCATCACAGACAAAGAGAAGG
CAGAGAAGCTGAAGCAGTCCCTGCCCCCCGGTTNGNNNCGTGAAGGAGCTGAAATGAACCACGCATGCTGCTTTGAACTGTATTAAATTTTTTAAATTCTCAA
AAAAAAAAAAAAAA
>‘000203a-005.scf” came from CONTIG 5 at offset 0;
“E:\SEQUENCE\export\EST_db\000203a\000203a-005.scf’ (45>563)
GCACGAGGCCAAGAATACAGTCACCTGCAGCCGGGGGACCACCTGACTGACATCACCTTAAAGGTGGCAGGTAGGATCCATGCCAAAAGAGCTTCTGGAGGAA
AGCTCATCTTCTATGACCTTCGAGGAGAGGGGGTCAAGNTGCAAGTCATGGCCAATTCCACGAATTACAAATCTGAAGAAGAATTTATTCGTATTAACAACAA
ACTGCGCCGNNGAGACATAATTGGAGTCCCAGGCANTCCCTGGAAAACCAANAAGGNCGAGCNTAGCGTCATCCCCTATGAAATCACACTGCTGTCTCCTTGC
CTGCACATGTTACCTCATCTTCACTTTCGCCTCAAAGACAAGGAACACCGTATCGTCAGAGATACTTGGACTTGATTCTGATGACTTGTGAGCAGAAGTTTAT
CTCCGCTCTNATAATCACGTTTTATNAAGTTCTTGNTGAATGGNNATTCTAAAATGTAACTCCATGAGAACATCATCCAGGGNAGCTGTGCTAGCTTTACACT
ACAA
>‘000203a-006.scf” came from CONTIG 6 at offset 0;
“E:\SEQUENCE\export\EST_db\000203a\000203a-006.scf’ (47>562)
GCACGAGGGTTTAATTAGTGTACAAGGAGGCTTCAAGAGGGCTTCTGTGGTGACCCCGTGGTAAAGCATCTGCCTACCAGTGCAGGAGACTCCAGTTCAGTGT
GGTCTGGGAAGATGCCACACACCCGGGGGAAACTGAGCCCATGTACCACAACTGCTGAGCCTGTGTTCTAGAATCCGGGGAGCTGGCACGAGAAGTCACAGCA
ATGAGAAGCCCACACACTACTANAGAGTAGCCACACTCACCACACAAGGCTTNCCTTGTGCTCAGTTGTTAGGAATCTGCCTGCATGGCGGAGACCTGGGTCG
ATTCCTGGTCGGAAGATCCCTGGAGAAGGAAAGCTACCTGCCGGAGCCACACGGAAGACCCACCTGACAGTCTGTGAAGAACTGAGAGCAGGGATAAACTAGG
ATCCTTGATTGTCAACTCTATCAAACAAACTCTTCTGTTTGTTTGCTTTCACACTTCTGCGTGCAAGCTTTTCCGCCCCTTTTNAAATAAATAATTTATTATA
TT
>‘000203a-007.scf” came from CONTIG 7 at offset 0;
“E:\SEQUENCE\export\EST_db\000203a\000203a-007.scf’ (32>465)
GCTGCAGGAATTCGGCACGAGGCTAGTTTCTTGTTTTTTTTTTTTTTTTTTTTTTTTTTTTTTTGCCTTTTCTTCCACTTTATTTCATATTCCCACCACAATA
ATGACTCCTTTAATTTAAACTAAAAACCATANAGGGTTCCCTGAAATTGTGGCAGCAAAGGAATGAAAGTGTCAAATACCGAGGGACAGGTGGGGTGGGGAAT
CACCGAATCGTCTCACTGGGCTCTTGAAGTTGCTGGCGGCTGAAGCTGCAGCTGGTAGGGCATTGATGGTATCTGAAACCGAAAGCCTGGGCCAACCTGGTGG
CGGCCCCTTGGCCGGTACTGGGGTGCACATGAAAACATTGAAGGACCCGCGCCGCAGAAGCGCCTCCGGGGGGGGGCCTGTTGATTGGGGGTACACCCCTCCC
CTGGGAAAAAATTTCCATGGCT
>‘000203a-008.scf” came from CONTIG 8 at offset 0;
“E:\SEQUENCE\export\EST_db\000203a\000203a-008.scf’ (39>747)
CAAAATGTCTTGAAATGATTACCATAATTTTAAGTAGGAAAGTTACCTGAACAGTTCTGCTTCCACTTAACTGACTGGCCCGCAATATTGTAGGAACAGCATG
TCCTTTGTACTGTGGTATTCAGAACAGCCACAGCACTCACTTTTTCCAAATGATTCTAGTAATTGCCTAGAAATATCTTTTTCTTACCTGTTATTTATTAATT
TTTCCCCATATTTTTATATGGAAAAAAAAATTGTATTGAAGATACTTAGTATGCAGTTGATAAGAGGAATCTGTTCTAATTATGTTTGGTGGATTATTTTTAT
ACTGTATGTGCCAAAGCTTTACTACTGTGGAAAGACAACTTTAATAAAGAATTACTTCCCAAAAAAAAAAAAAAAAAAAAAAAATAACCGGAGGGGGGCCCGG
TCGCCATCGTCCTATGGGAGCGTTACCATCCACGGGCGGCGCTTACAGCNCGGACGGGAACCCGCCGTCCCCACCTACGCCTGCACCCACCCCCTTCCCGTGG
GTAAAGGAAAACCCCACCACGCCTCCACGTGCGCACCGAGGCGAGGAAAGAAGGGTAATTTGTAATCGTAATTTTTATATATTTTACATGCCAAGCCATCCTA
AAAAAAAAAGAAGGGGGGGGTGTGTAAAAACTCTTAAAGCCCCCGAGAAAAACCTAGGGCCCCCCCCCCCCTCTTTGGGGGCGAC
>‘000203a-009.scf” came from CONTIG 9 at offset 0;
“E:\SEQUENCE\export\EST_db\000203a\000203a-009.scf’ (37>606)
TAATTCGGCACGAGGCTCATTTTTCCATCTCTTATGAGGACACACATCACTGAATTTAGGTTACCCTAAGTCCAGAATAATCTCATCTTGAGATCCTGAAACT
TATCACATTTGCAAAATTCAAGAGCCAGGGAAAGCTGAGCAGTGACTGCTAATGGAACAGGGTTTGCTTCGAGGGTGATGAGAGTGTTCAGGGGTAGACAGGG
ATGCTGTTTGTACGACTCAGTGAATATACTAAAACCCNAGGGATTGCATGCTTTAAAGAAGAAGCTTAATGTTTGTGAAATTAGTCTCAATATAGCTGTTATT
TTTAAAAGAGCCTGGCTCGGGGAGCCATCAATCATACTGCTATTTTTATATCGATGTGCCAGCAGAAGTATTCTTAAATCTTTATGACACTGTTTTACTTTTG
GCTGTCTCCCACCTGGTTTAAATACATTGAACAGAACCCAGNGAAAGCCTATGGTACAGGGAGAGCCCCGCTTGCCATGAGGGATAGAATTGGTGATGCCAGA
GATTCCAAGAATTTGTGAAAAACCAGACTCCGGGGCCGGGTAACCTTCTCCG
>‘000203a-010.scf” came from CONTIG 10 at offset 0;
“E:\SEQUENCE\export\EST_db\000203a\000203a-010.scf’ (44>427)
GCACGAGGAGAGAACTAGTCTCGAGTTTTTTTTTTTTTTTTTTTTAACTGAAGGAAAATTTCTTTACAATGCTGTGTTGGTTTCTGTCATACCAACGTGAATC
AATCATAATTATATTATATATCCTGATGGCACATGTTAAGAATGCATTTTCTCGTTTGAACATTACTGAGTTGGGAGATATGCAGGTTATGGATTAGTCTCTC
TTGTGACTACTGACTTAACTAAAATTCAGAAGATACAGCCATTTACCTACAGTCCTCCAGTTAAAACATGGCAGACCTGAGCCTANAACCCAGTTTGCTCATT
TTCACTCCAGTATGACCCAACTATACCAAAATGTTCCCTCTGCAGATACTATTCAAAGCACTTTATTTATTTCTAT
>‘000203a-011.scf” came from CONTIG 11 at offset 0;
“E:\SEQUENCE\export\EST_db\000203a\000203a-011.scf’ (31>278)
CTGCAGAATTGACACGAGCACCCTCTCAAGACCGAGCTGCTGCGGCCACACTCCTACAGTCTGTGCAAGCCCCCGAGTTCACCCCCAAGTCTGGAGGGAAGAA
CCATGTCTGTGACCAGCAACTGCAAAGAGCCAATGCCTGTGTGGTTGACAGCCGGCGTGGAGATCTCATAGCTACTGTTGCCACTGCCCGACAGCTCCCTGGC
TCAAAAATTACCCTCATCTACTTGATAAGGATGATGTACACC
>‘000203a-012.scf” came from CONTIG 12 at offset 0;
“E:\SEQUENCE\export\EST_db\000203a\000203a-012.scf’ (1>719)
TGATGCCTTCTAATTATGGTTCCCCCGGGCTGCTGGTGAGACCTGTGTATACCCCACGCCCTGTGTGGTTCCAGAAGAACTGGTATATCAGTAAGAACCCCAA
GGAAAAGATGGCTCGTCTGGTACGGAGAGAGCATGACCGTCGGATTTCAGTTCGAGTATGGCGGCCAGGGGTCCGATCCTGCCGATGTGGCCATCCAGCTGAC
TTTCCTGCGCCTGATGTTCACCGAGGGTCTTCCATAACATCACCCTACCACTGCAAGAACAAGAGTGGCCTACATGGGACCAACTGACTGGCAACCCTCAAGA
TGCCCTGCTCCTCCAGGGCTCCAACGAAGTACGAAATCCGGGCCGAGGACAACAGCCGCTCCACTACAGCGACACCTAAAATGGCTGCACGATCACACCGGAC
CCTGGGCAAGAAGAGACGAATACAAACACCAAACTCCGCTGCCCACATGATGGCCCCTTGAAGTGGCGCCCATACAGAATTCGTTCGAGTGGCCGCCTGTTCT
GAACTCCTTTCCCCACCGCTCCTCACCAACCCTGCCCGACTCGAAAACAACACCAACGAACCCAAACAAAAGGAAAATCACAGCTGAAAATTTTCTGCTTTCT
TAATATTTATTTACACAACTACAACAAAGACACTCAAAAAAAAACAGGACGCCCCCCCTAGGCATAATATCGGTTACGGAGGACCGCCCCCTCCTCCC
>‘000203a-013.scf” came from CONTIG 13 at offset 0;
“E:\SEQUENCE\export\EST_db\000203a\000203a-013.scf’ (284>351)
ATGAAGCGTTATATTTTGTTAAATTCCGTTATATTTTGTTAACACCTCATTTTTTACCCATAAGCGC
>‘000203a-015.scf” came from CONTIG 14 at offset 0;
“E:\SEQUENCE\export\EST_db\000203a\000203a-015.scf’ (1>680)
CTGCGGCCCTCTACACTATGGATCCCCGGGCTGCAGGCGGAAGATGGCGGCCACGGCGGTGAACGGGGTGGCCGGCACCTCGAGCTCGGGGTCTGCGGCGGCC
TCGGGCGCGATCCTGCAGGCCGGGGCCGGCATGTACGAGCAGCTCAAGGGCGAGTGGAACCGGAAAAGCCCTAATCTTATCAAGTGCGGNGGAAGAGCTGGGG
CGTCTCAAGCTGGTTTTGTITGGAGCTCAACTTCCTGCCAACNNCACAGGACCCAAATGAGCAAGCAGCAGCTCATTCTGGCCCGTGACATACTGGAGATCCG
GGCTCATTGGAGTATCCTACGCAAGGACATCCCGTCCTCGAGCGGGACATGGCCCAGCTCAAGTGCTACTACTTCGATTACAAGGAGCAGCTCCCAGAGTCAG
CCTACATGCACCACTCCTGGGCCTCACCTCCTCTTCTGCTGTCCCAAACCGNTGGCTGATTCCACCAGACTGGACGGTGCCTGCCAAGACATCCAACCACGGT
ACACAAGCATCGGGNCCTCGAGCATACGAGGAGGCAGTACATAGTATTCTGGCAAGCACATCCCGCGAACTACCTTCTCATGATTCGCTGAACTCAGAAGAAG
TTGTGANGAAGGCATGAAATCTTTACAAGCCCGACCCCCTCACAAGCAAAAAAAAAAACCAAA
>‘000203a-016.scf” came from CONTIG 15 at offset 0;
“E:\SEQUENCE\export\EST_db\000203a\000203a-016.scf’ (8>560)
GCTCTATACTATGGATCCCCGGGCTGCAGGTTCGCTTAGGCGCAGACGGGCAAACAGAGCCAGCATGCCGGTCGCCCGGAGCTGGGTTTGTCGCAAAACCTAT
GTGACCCCGCGGAGACCCTTCGAGAAGTCCCGCCTCGACCAAGAGCTGAAGCTGATCGGCGAGTATGGGCTCCGGGACAAACGTGAGGTCTGGAGGGTCAAAT
TCACCCTGGCCAAGATCCGAAAGGCTGNCCGGGAGCTGCTGACGCTGGATGAGAAAGACCCGCGCGTCTGTTCGAAGTAATGCCCTGTGCGGCGGCTCGTCGG
TATCGGGTGCTGGATGAGGCAAGATGAAGCTGGATACATCCTGGGCTGAAGATGAAGATTTTTTGAGAGACGCCTGCAGACCAGTCTTCAGCTGGGCTGCCCA
GCCATCACCAGCCCGGGCTCTCCGCACGCACACAGGTCGCAGCAGGGAGACATCCGTGTCATGGCGCTGGACTCCAAACCATCACTCTCCTCCTCCCCTCGCG
GGGCGNCCGGCCGGGAAGAANAAGCAAAGACAGGGGGT
>‘000203a-017.scf” came from CONTIG 16 at offset 0;
“E:\SEQUENCE\export\EST_db\000203a\000203a-017.scf’ (44>531)
GCACGAGGAGTGACCAGGGTTAGCTGGGATGCCCTCAGACTGCACTGGACCAGCCCCGATGGGATCTATGAACGGTTTGTCATTAAGATCCGGGAGACTGACC
AGCCCCAAGAAGTTCACAGTCTCACGGTTCCTGGCAGCCAGCACTCCGTGGAGATNTCCAGCCTCAAGGCTGGTACCTCTTACACAATCACCCTGCGTGGCGA
GGTCAGGGACCACAGCACTCAACCCCTTGCTGTGGAGGTCATCACAGCGGAGGTCCCCCAGCTGGGAGACTTATTCGNGACTGAGGCTGGCTGGGATGGCCTC
ANACTCAACTGGACCGCAGCTGATCAGGCCCTTGAGCACTTTGTCATTCAGGCGCAGGAGGCCACAGGGTGGNAGGCGCTCAAACTCCCGGGGCCCAGGACAT
GCGGCTGGGACATCCGGGCCCTGAGCGCNCCCCTACAGAGCACATCCACGGTGATCGGGCTATAGACCAGGCTCTT
>‘000203a-018.scf” came from CONTIG 17 at offset 0;
“E:\SEQUENCE\export\EST_db\000203a\000203a-018.scf’ (13>586)
AAATATGGATCCCCGGGCTGCAGNAATTCGGCACGAGGGTACCATCTATTTTTTCAAACCTGGCAGGAATCCCCGGGGGGAAGCCCGCATACTCCTTCCACGT
TACCGCAGATGGTCAGATGCAGCCCGTCCCCTTCCCCCCAGATGCCCTCATCGGCCCTGGCATCCCCCGACACGCTCGCCAGATCAACACCCTGAGCCATGGA
GAGGTGGTGTGTGCGGTGACCATCAGCAACCCCACGCGACACGTGTACACGGGTGGGAAGGGCTGCGTCAAGGTCTGGGACATCAGCGACCCCGGCAACAAGA
GCCCGTCTCTCAGCTCGATTGTCTGAACAGGGATAAACTACATCCGTTCTGCAAATTGCTCCCTGATGGCTGCACTCTCATAGTGAGAGGGGAAGCTAGTACC
CTGTCCATCTGGGACCTGCGGCTCCCACCCGCGCATCAAGCAGACTGACGCCTCGGCCCCGCTGCTCGCCCTGCCATCAGCCGGACTCAAGTCTGCTCTCGGC
TGCAGCGAGGCACATGCTGGTGGGACTGCACACCAACGCGTGAGGCATNCAGGCACCGA
>‘000203a-019.scf” came from CONTIG 18 at offset 0;
“E:\SEQUENCE\export\EST_db\000203a\000203a-019.scf’ (13>287)
AAATATGGATCCCCCGGCTGCAGNAATTCGGCACGAGGCAGGCCTTTTTTTTCTCTCTCAGACAACCATCTCATGGACCCCATTCCAGGAAAGCTCTGAGTAT
ATCATTTCATGTCATCCAGTTGGCATTGATGAAGAACCCTTACAGTTCCGAGTTCCTGGAACCTCTGCTAGTGCCACCTTGACGGGCCTCACCAGAAGGGCCA
CCTACAACATCATATGGNAAGCAGTAAAAAAACAANCAGAGCAGAAAGTTCGCGAGGAGGGGGTTNCCG
>‘000203a-020.scf” came from CONTIG 19 at offset 0;
“E:\SEQUENCE\export\EST_db\000203a\000203a-020.scf’ (2>215)
CTTCCGTTATACTAAGGATCCCGGCCGCGGAATTCGGCACGAGCCTCAGTTTTTTTTTCAGCCTCAGGCCCACCCTGAGGGTTCCTCCTCCAAGCTGGCATCG
CCCCACTTTACAGATGACCACCCCAGGCTTGGACAGGGCCGCCCCTGGACAAGAAGCTGATCAAGGCCCTCTTTGACGTGCTGGCGCACCCCCAGAACTACTT
CAAGTACA
>‘000203a-021.scf” came from CONTIG 20 at offset 0;
“E:\SEQUENCE\export\EST_db\000203a\000203a-021.scf’ (29>265)
CCTGCAGAATTCGCACGAGGAGAATCTATTTTTTCTCTTGATGAGGGTGAAAGAGGAAAGTGAATAAGCTGGCTTAAGACTCAACATTCAAAAAACTAACATC
GTGGCATCTGGTCCCATCACTTCATGGCAAATAGATAAGGAAAAAGTAGAACGGGGTCAGGCTTAATTTTTTTGGCTCCAAAATCACTGCAGATGGGGGTTGC
AGCCATGAAATTAGAGATGCTTGCTTCTTG
>‘000203a-022.scf” came from CONTIG 21 at offset 0;
“E:\SEQUENCE\export\EST_db\000203a\000203a-022.scf’ (36>646)
AATTCGCACGAGGTGGAAGCTTTTTTCGGGGGTTGTGACTGGGGGCCGGAGTGCCCCACCCGATTGGTGGGTCCCCTTCCGCATTTAGGGTCCCTGAGCATGC
TTTCTTGCCAGGGAGCTGGAAAGTTTTCTGACCCTTTTCCCCAGAAAGAGAGACAATAGATTGCCTTCATTTTGATGTCTGTGGCCTCAAAATTGATCATTTC
CTGTCTCCTCCCTCCCTCCCCGCCCTGGGGCCCCCCGCCGATITCATCCCCACCCCTCCAGAGCCACTTANGACCCACTTCTGACTAATTATGGATTCCAGAT
GCTTGGGATAAAAGAAAAAGGACCAAGAACCCCTCCCCCTCTCTGACCTGGCCAAAGCCCTCCCCCAATCCCCAGGTCTCTGGAGGGCTCTGCTTAAGCCCGC
CTCACCGANAGNAGGNATGTAGCTGTAGAAACAACCATGCAAACTGGGTGGCCTGCAGTTACACCACCCAATCTTCCCTCCTGGCTCCTTACATGATGAGGAC
AACTGGCTGAGAAGGGCGCAAGCGTCTGGCTCACTGCTATTCTGAAATAGAACTGGCTCTTGCTGGCGTGGCCTGGGTAGGGCCGGCAGAGGGG
>‘000203a-023.scf” came from CONTIG 22 at offset 0;
“E:\SEQUENCE\export\EST_db\000203a\000203a-023.scf’ (1>640)
GTGCGTTAAATAGGATCCCGGCTGAGAATGCCGAGGAAAAGGCCAGGTTAAAAGGGCCCGGCCCGGCCGGGTGAAGAAGCAGGAGGCCAAGAAGGTGGTCAAC
CCCCTGCTCGAGAAGAGGCCCAAGAATTTTGGCATTGGACAGGACATCCAACCCAAGAGGGACCTCACCCGCTTTGTCAAATGGCCCCGCTACATCCGGCTGC
AGCGGCAAAGGGCTATTCTTTATAAGCGCCTGGAAGTGCCTCCTGCAATTAACCAGTTCACGCAGGCCCTGGACCGACCAACAGCTACTCAACTGCTTAAGCT
GGCCCCAAGAACAGACCACAGACAAACAAGAGAAAAAGCAGAGCTGCTGGCCGAACTGAGAAAAAAGCGCGGGCAAAGGCGAGTCCCTACCAGAGCCCACTGT
CCTTCGAGCAGGTNCACACGGCCACACCTGGGGAAGACAGAAGCTCAGTGTTGTGATCGTCAGAGTGGTCCCTTGGCTGGGGTCTCTGCTGCCTGGGCGCAGA
GGGGNTTCTATGCTATAAGGCAGGCGGCTGGCGCGTGCCAGAGACGGCCACGACTTTACCATCACGGGAAAGAGCTGTTAGGGGAACCAGACATTAAACAAAA
AGAACGGGTGGGGGAGGCGGGCA
>‘000203a-024.scf” came from CONTIG 23 at offset 0;
“E:\SEQUENCE\export\EST_db\000203a\000203a-024.scf’ (1>602)
CGCCGCCGCTTAAACTATGGATCCCCGGGCTGCAGNAATTCGGCACGAGGATTAGAAGCGGATAGAAGGGCTAAAAATCAAAGGCGAAGAGTTCATATGACTC
TGGAGTTACCATGGAGTGCTGATAGAGCAATTCAGCAATTTGGACGAACTCATAGATCAAATCAAGTTACCGCTCCTGAATATGTCTTTCTGATTTTCTGATT
GGCAGAAGAACAAAGATTTGCATCTATTGTTGGTAAAAGACTTTGAGAGTTTGGGCGCACCTACACATGGAGACAGAAGAGCAACAAAAACTAGAAAACCGAG
CCGCGTCCACCTTCGATAATAAGATGGAAGAAAAGCTTTAAAAATGTGATGAAATCCAATGTGAAACCAAATTCCTTGGTTCACCACTCCAGACTATCCTGGA
GATTCTTTAAGAGTTCGCAAGACTGATAGAGTGTCTTATAAAGTGAAAAAGTCAGAATCTTCTTTTATAAAATTTAAAACAAAGNAAATTTTAACAATTTGGG
CGGGAGGGCCCACAAAGCCTTTTTTTTTCGCACCCCTCCTGCGGCTCAAAGCAAAAAAGAAAAACATAGGAATTAATGTTGTTG
>‘000203a-025.scf” came from CONTIG 24 at offset 0;
“E:\SEQUENCE\export\EST_db\000203a\000203a-025.scf’ (29>176)
GGGCTGCAGCTCCATGGGGTGTTGGTGCCTGCCAGCCACGGAGGCCGGGCGGCCAGAACGCGCACAGAGGGATATGATATGGTCCGGTGTGATGGAGAGAGCA
AGCGGGACCGTGCAGCCTCCCAGGACACTGGCCCCGCGGGGAGCC
>‘000203a-065.scf” came from CONTIG 25 at offset 0;
“E:\SEQUENCE\export\EST_db\000203a\000203a-065.scf’ (1>665)
GTGGCGCCCTCTAAACTATGGATCCCCGGGCTGCAGGTGGACTACACCATCACTGTCTATGCTGTCACCGGCCGGGGGACAGCCCGGCAAGCAGCAAGCCCGT
TTCCATCAATTACCGAACAGAAATTGACAAACCATCCCAGATGCAAGTGACTGATGTCCAAGACAACAGCATTAGTGTCAGGTGGCTGCCTTCAAGTTCCCCT
GTTACTGGTTACAGAGTGACCACTGCTCCTAAAAATGGCCCAGGACCATCGAAAACGAAAACTGTAGGTCCAGATCAAACAGAAATGACAATTGAAGGGCTGC
AGCCCACAGTGGAGTATGTGGTCAGTGTCTATGCTCAGAATCAAAACGGAGAGAGTCAGCCTCTGGTTCAGACAAGCGTTACCCACCATTTCTGCACCAACCA
ACTGAAATTNACTCAGTGACACCACCAGCTGACTGCCAGGACGCACCNATGTCACTCAGTGGTTCGAGGCGGNGACCCGAGAAAGACGNACGAGAAGAATCAC
CTGCTCTGAACTATCGGTfGTITCAGACTAGTTGCACCAATGAGGAGGCTTGCTTTAGACCTGACACAACGCTAGGAGGTCAATTGAAAGCACTCAAAGGCCG
GGAAAGTTGAACACTCCTTATGAACAAATGAAAACGGTCAGTGGCTCC
>‘000203a-026.scf” came from CONTIG 25 at offset 40;
“E:\SEQUENCE\export\EST_db\000203a\000203a-026.scf’ (38>628)
AATTCGCACGAGTGTCTATGCTGTCCCGGCCGGGGGACAGCCCGGCAAGCAGCAAGCCCGTTTCCATCAATTACCGAACAGAAATTGACAAACCATCCCAGAT
GCAAGTGACTGATGTCCAAGACAACAGCATTAGTGTCAGGTGGCTGCCCTCAAGTTCCCCTGTTACTGGTTACAGAGTGACCACTGCTCCTAATAATGGCCCC
AGACCATCGAAAACGAAAACTGTAGGTCCAGATCAAACAGAAATGACAANTGAAAGCTTGCAGCCCACAGTGGAGTATGTGGTCAGTGGCTATGCTCAAAATT
CAAAACGAGAGAGTCAGCCTCTGGGTCAAACAGCGGAACCACCATTCCTGCACCACCCACCTGAATTACTCAGNGAAACCAACAGCTGACTGCCCAGGNACGC
ACCCATTTCACTCACTGTTCGAGGCGGTGACCCGAAGAGAGACGNACGAGAAAAATCACCTGCTCTGAACTATCGGGTGTATAGACTAGTTGCACAAATTAGG
AGGCTAGTCTAGACCTGAAGCAACGCTCAGATGTACATTGAAAGAGCCTCAAAGCCGGGAAAGCTTGAACCCTACA
>‘000203a-027.scf” came from CONTIG 26 at offset 0;
“E:\SEQUENCE\export\EST_db\000203a\000203a-027.scf’ (30>646)
CTGCAGAAATTCGGCACGAGGAGCGAGTCCTGTTAGGTGCGCGTGGAAACTAGGGTCATGGCTGCGCCCGGTCCAGCGCTCTGCCTTTTCGACGTGGACGGGA
CCCTGACGGCCCCGCGGCAGAAAATTACCAAAGACATGGATTGCTTTCTGCAAAAACTGAGGCAGAAAATCAAAATTGGTGTCGTCGGCGGGTCGGACTTTGA
GAAAGTACAGGAGCAGCTGGGAGATGACGTTATTAAAAAATATGNTTACGTGTTTCCAGAAAATGGCTTGGTAGCATACAGAGATGGGAAACTCTTGTGTAAA
CAGAATATTTAAGGTCACCTGGGTGAAACCCTAATCAAGATATATTCACTACTGTCTGAGCTACATCGCGAAAAATCAGCTCCNGAAAAAAAGGNCACTTCAT
AGAGTCCGTAACGTGAGCTGACGTGTCGCCGACGGAAAAGCTGCAGCAGAANAACCATGTATCTACGACTGTACAAAAGAAACATAAACAAAGTCGGAGNATT
GCAAAGATTGCTGTAAGGCTACGTTTTTCTAGAGNCAATCACTTATTCTCCCTAGCTGAACAATACGCTGGAACGGGAAGAAGATAAACTTATTTTTGGACAA
C
>‘000203a-028.scf” came from CONTIG 27 at offset 0;
“E:\SEQUENCE\export\EST_db\000203a\000203a-028.scf’ (40>622)
CACGTAGGGGCGACCGCAGGCCCTCTCCCGAGGAGCTGGACAAGGGCATCGACCCCGAGAGCCCCCTGTTTCAGGCCATTCTGGACAACCCCGTGGTGCAGCT
GGGCCTGACCAACCCGAAGACCTTACTAGCATTTGAAGACATGCTCGAGAACCCGCTGAACAGCACCCAGTGGATGAACGACCCGGAGACGGGCCCGGGCATG
CTGCAGATCTCAGAATCTTCCAGACCCTGAACCGCACATATGCCGCGCACTGCAGCTGCCAGCCCAGAGAGCCTCTFCTFITCCCAGCCCAGGGGTGGGGAGA
GGGTGCAGACCCCAAGGTGCGCCTGGGCTGGGGGCGGGGAGCAGGGGGGCNTGGAGGGACCCTGCCCCTGGGTGTGGCGCCAGGCCGCACTCCGCTGGATCTT
CTGGAAAAACTCGGNGGCAGGGCCGGGTGGCTCCACCCCTGACAGGTTACGACAGGCGCCACCGGGAAGGGGGCTCCTTCAGGCCCTGGCTCTGACGTATTGA
TTAACGAGCGCGCTGGAAGACCTGTTTGAAAAAGAATGTCAACCAGTTAGGAAGGATAATGGGAAAAAAA
>‘000203a-029.scf” came from CONTIG 28 at offset 0;
“E:\SEQUENCE\export\EST_db\000203a\000203a-029.scf’ (35>595)
AATTCGCACGAGGTCATCCCTAAGTGGCCTGAAGATGGACAAAGGGAAGTAACAGGCACGTGATGTTGGCAAGGATGCTTCTAGGGCTAGAGGATCAGTGGTG
GGAGAGAGCTGCAGAATCCACCAGCCAGAACTGCAGATAACGATATCTATGGTCAGGGGCTGTGACTGAGAGAAGGAAACTGAGGTTGTGTTCTGAAAGTACA
TAAACTCTCACATATACCCAGTTCTTCACCATCTTCCCTCCTCACTTTGCAGNGCCATTTTTTTTTGCATTAGGCAAATTGCTCAGACTTCCAGAGCCATGCC
CATCCCGTCTCTGGAACCCCCACACCTCTGAGAGTGGGATCACCACGTCCTGCAGGGCTGCTCCCCTCCAAGTACCTTTAGAGAGCAGGACAGGAGCTGTTTC
ACCACAAGAGAAAATCAAACGAGAGCAGACGGGTAAACAAANAAGACAGGGGCAATGTTTTCTTGNGTTTTGTTTTTTTTTCCATTGGAGGTGACACAAAAAT
TCAAGCTACAGTTCCCCTCTCCCCCCGATTTTTTTTTAACAAANA
>‘000203a-030.scf” came from CONTIG 29 at offset 0;
“E:\SEQUENCE\export\EST_db\000203a\000203a-030.scf’ (36>676)
CTCATCCTACCATATAGATATTGGTACCCTTTATCTACTATTTGGTGCTTGGGCCGGTATAGTAGGAACAGCTCTAAGCCTTCTAATTCGCGCTGAATTAGGC
CAACCCGGAACTCTGCTCGGAGACGACCAAATCTACAACGTAGTTGTAACCGCACACGCATTTGTAATAATCTTCTTCATAGTAATACCAATCATAATTGGAG
GATTCGGNAACTGACTTCCCCTAATATHGGTGCTCCCGATATAGCATTTCCCCGAATAAATAAATAAGCTTCTGACTCCTCCCTCCCTCATTCTACTACTCCT
CGCATCCTTATAATTGAAGCTGAGGCAGAAACAGCTGAACCGNGNACCCCTCCTTANNCAGCAACCTACCATGCAGGAGCTNATAGAACTACCATTTCTTTTC
ACTTACANGAGTCCTCATTTTAGAGCATCAACTTCTACACAATACAACATAAGCCCCGCATGCCATACAACCCTTGTGTTGATCGNATATACGCGACTATATA
YFGCTCTTTTACACGCACCAGCTTTAAACGAACTATCACTCTCACGCGAGAGAACTTTTTTACATTTTGTTTTGGCCCGAGCTTTTTTTCTGGGGGATTCTTT
GCCCCTCAAAAAAACAGTA
>‘000203a-031.scf” came from CONTIG 30 at offset 0;
“E:\SEQUENCE\export\EST_db\000203a\000203a-031.scf’ (13>195)
TACTATGGATCCCCGGGCTGCAGGNAGTTTTTTTTTTTTTTTTTTTGTACAAATCAAGCATTTTATTACATAAATAAAAGCAGCACGCTTTTATTTTCTATTT
AAATACCATACACGAGATTTAAAATCACATTTGGCAGTGGACTGCAGGATGCTCAGACTTCACCCACATCACNTTGGATT
>‘000203a-033.scf” came from (F3,033)
no description length
779GGCGCCCTCTAAATATGGATCCCCGGGCTGCAGGAAtTCGGCACGAGGCCGGACCGGTGTCCttCTCTGGAGGCTCCTCGCTGGTCGTGGGGGAGCCGGG
AGGGCATGGCTGGCTGCCCGAAAGAGACTGCGAGACGGTGACCTGCTGTCTCTTTTCGGAGCGGGACGCCGCCGGAGCTCCCCGAGAAGCCGGCGAACCCCTG
GTCGGGGCGGCCCTAGAGCCAGAGGCGGTGGGCGGGAGCGCGAAGCCCGCTCGCGGGTGCTGCTGCTGTAGCAGGAACTCAAGATGGTCACGATCTCGCTGCT
GAAGCGGCTCAAGGAACGCTCGTTGGCCACGCTGCTGGAGGGGGGAGACCCGCGGGGGGGGCCGGGCGGCTGCGGGCTGTGCCCGCCGCCGACCTCCCCTGGC
GGCCACCCCGCCGCGCACTGGTGTCGGACCCCCTCCCTGGCCCACCTCAGCCGCCGGGGCCCAGCCCTGGCGGGGTCCACTCCCCCCCCCCGAGGCCCCAGGG
CCGCACCCCACCCTCACCGCTCGGGGCCAAATACCGCCCGCCTACCTGTTTCCCCGGACAACAACACGATTATTATCCCTTGCTAATGAAAAACGCCCCCTTT
CATCCGGAATCAACCACTGCCGCCCCCACCACCTGGCGGGGCTGTACGGCGGGGGGCTCCCTCCCCCCCCCCATTT7CTCTCTTTTTTTTCGTCTCATTACAT
TTGGGGGCTATATATATAATATATTATTATAGATATTATTTTTTTTTCTATCTATATTTTTA
>‘000203a-034.scf” came from CONTIG 31 at offset 0;
“E:\SEQUENCE\export\EST_db\000203a\000203a-034.scf’ (28>623)
GGGCTGCAGAATTCGCACGAGGCTGTCTGCTCGTGGTGGAGATGGCAGTAGGATCATTTTTGATGATTTTCGAGAAGCGTACTATTGGCTTCGTCACAATACT
CCAGAGGATGCGAAGGTCATGTCATGGTGGGATTATGGCTACCAGATTACAGCTATGGCGAATCGGACGATTTTAGTGGATAATAACACGTGGAATAATACCC
ATATATCTCGAGTAGGGCAGGCCATGGCATCCACAGAAGAAAAAGCCTATGAGATCATGAAGGAGCTTGATGTCAGCTATGTGCTGGTCATTTTTGGNAGCCT
CACTGGGATTCTTCAAATGACATCAACAAATTTCTGTGGATGGGCCGGATTGGAAGGAGCACAGATACAGGAAACACATACAGGACACGATATTATACTCCAC
TGGNGATTCGNGTGGACCCGGAGGCTCCCANGCTGCTCACTGCTTTAGACAAAAGGGTACTACGATTGACAGGNTACCAAAACCACGGCCCTAGCTTTACCGG
CCGGATGCGAGATGGAATAAACTCGACTGAGTCTAAAAACAACACACCACATGCTGGCGAATTCAAGGAGACCGNAATCAG
>‘000203a-046.scf” came from CONTIG 32 at offset 0;
“E:\SEQUENCE\export\EST_db\000203a\000203a-046.scf’ (37>597)
NAATTCGGCACGAGGGCATGAATGTCCTGGCCGATGCTCTCAAGAGTATCAACAATGCCGAAAAGAGAGGCAAACGCCAGGTCCTTATTAGGCCGTGCTCCAA
AGTCATCGTCAGGTTTCTAACAGTGATGATGAAGCATGANTACATTGACGAATNTGAAATCATTGATGANTCACAGAGCTGGAAAATTGGTGNGAACCTCACN
AGCAGGGCTAATAAGTGTGGAGNGATCAGCCCTAGATTGATGTGCAACTCAAAGATCTAGAAAATGGCAGAATACCTGCTCCATCCCGCAGTTGGTTTCATGT
ACTGACAACTCAGCTGCATCATGGACATGAAGAGCAAGACGAAACATACAGAGGAAATCTTGATTCTTTTTAGGAGTATACTACAATAATGCTCAAGACTTGT
GCTTCTTAAAAAAAAAAAAAAAACGACCGCACTGATGACAGATCTACATATTCTGACCTTTTTATCTCACTAAAGTCAACCACTTTTCCATCAACGAACACAA
AATAAAAAAAACCCTGAAAAAAAAAAATTTTTTTTTTTTCTTTTTT
>‘000203a-046.scf” came from CONTIG 32 at offset 0;
“E:\SEQUENCE\export\EST_db\000203a\000203a-046.scf’ (37>597)
TTTTTTTTTTTCCTTTCTCGCTCCCTTCCTTTCTTCCTTACTTACTTCTTTGCTTTTGGCTGCATTTTCTTTAAATTCGACACAGTTATGTTAAAAAATATAT
GCATTTGTACTTAGAGTTTGGTGTAATTTAAAATATGTGGAGTGATTTCATTCACTCTCCTGTTTTAAACATTTGTTAAGGACTCAGCATGTGAAGGAGCAAG
AGATATAGTCATTTTTATTAGAAAACTTCAGTGTTCTAATTTGATCAGAGACCGNGAATAATCAGAAGATGACATGATTTACTTGGAATATACAGCTTATCAA
GGACTTCGUTTATGATGGTTATTTAAAATC
>‘000203a-037.scf” came from CONTIG 33 at offset 0;
“E:\SEQUENCE\export\EST_db\000203a\000203a-037.scf’ (37>554)
CGCAGCCACTCCGACCGGTGCCGCCTCGTCCTGCTTCGCCATGACTTCCTACAGCTATCGCCAGTCGTCGTCCACCTC5GTCCTTCGGGGGTATGGGCGGCGG
CTCCATGCGCTTCGGGGCTGGGAAGCGCCTTCCGCGCGCCCAGCATCCATGGNGGCTCAAGTGGCCGCGGCGTGTCGGTGTCCTCCGCCCGCTTCGTGTCCTC
GTCCTCCGGGGGCTACGGCGGCGGCTATGGGGCGCCCTGGCCACCTTCGACGGGCTGCTGGCGGGCAACGAGAAACTCACCATGCAAAACCTCACGACCGCCT
GGCCTCCTACCCTGAGAAGTGCGCGCCCTGGAGAGCCAACAGCGATTGGAGTGAAAATCGCGACTGGACCAAAACAAGGCCGGCCCGCCGCGACTACACCTAC
TCAAACATAAGACTGCGNACCAACTCGTGGCACATGAAACTGATAATCTGCATACACAGCCGTCGCTGCAAGACTCGCACATTGAGACGACAGCTGGCAGAGG
GAGC
>‘000203a-038.scf” came from CONTIG 34 at offset 0;
“E:\SEQUENCE\export\EST_db\000203a\000203a-038.scf’ (38>594)
NAATTCGGCACGAGGAGCAGATCCTGGCTGCCCTCGAGAAAGGCTGCAGCTTCCTGCCGGACCAGTACCGCAAGCAGTGTGACCAGTTTGTGAGGGAGTATGA
GCCAGTGCTGATAGAAATCCTGGTGGAGGGGATGGACCCTTCCTTCGTGTGCTTGAAGATTGGAGCCTGCCCAGCAACCCACAAGCCGCTTTTGGGAGCTGAG
AAATGTGTCTGGGGCCCGACCTTACTGGTGCCAGAACATGGAGTCGCAGCCCTGTGCACCGCGTCGAGCACTGCAGCGTCACGNGTGAAACTAGGGCACGCTT
CACCCTGAAAAACTGCAGCGTCTTTTCTGCTCGGTTGTCTGGGGTAACCACACCAATTGTGACTTTGTATAAAAAAGACCCTTCCTCATCCTTNTTCTCCCTC
TTGTGCGTGCTTGCAGGCAGTGACTGCTGTTTCGTCCTTTTTGTAAAAAGCGAACCTCCTGAGTTTTGATTGTGGCGGGGTAGGGGAAAGGGTTGTCGGAGGA
ACGACCTCGCGAGGCCGCCCCGCTGTTGGGGGGGCCTGCGCT
>‘000203a-040.scf” came from CONTIG 35 at offset 0;
“E:\SEQUENCE\export\EST_db\000203a\000203a-040.scf’ (29>585)
GGGCTGCAGGTTAATTCATTTTTCTGGAAAAAGAGAAGATGTTTATTTATTTTTCCATGGTAAATTCTTTTGAATCTGCCTCTTAAACCTAACTCTGGGCTCT
CTCAGGAGGGGCAAAGAGGACCTTTGAGTTAAAACCCTCCAATGGAGACCCTGGGAAAGAACCGGAGGCATAACACCCNAGCCGCCCTCCAACTGGACTGTAN
GACTCCCCAGACCCGCTGCCCAGCTGCTTCTGCCCATCGNTCTGCCTGGTTGGGTTNTGGGTCCTGGATCCCACCCGAGCCCTGTAGGATGGCACCACAAGCC
CTACATGAAGAGCTTTGTGGTGTCACTAAAATGTGTGTTTCGGCACGTTGCTGTCATTCTGCCTGNCTGCCATGCTGAAAAGCTGGCACAGCCCGANAAGCCA
GCGAAAACACCTTCTGCCAGANCTCTGNCCCACTCGAGATGAGACCACCAGCTGCTGTCCTCCCAGAACAGGTATTATATTTAAGTAAAACTGTTACTAAAAA
GTTTGTTCCAACTTATTCAAAACAAGAGAAAAGGGGCGT
>‘000203a-041.scf” came from CONTIG 36 at offset 0;
“E:\SEQUENCE\export\EST_db\000203a\000203a-041.scf’ (1>593)
GGGGCCCTCTAAACTATGGATCCCCGGGCTGCAGNAATTCGGCACGAGGCGGACCTGCTGGAGCTCCTGGCACTCCTGGACCTCAGGTATTGCTGGACAGCGT
GGTGTGGTCGGCCTGCCTGGTCAGAGAGGAGAAAGAGGCTFFCCCTGGTCTTCCTGGCCCCTCTGGTGAACCCGGCAAANCAGGTCCTTCTGGAGCAAGTGGT
GAACGTGGCCCCCCTGGTCCCATGGGCCCCCCTGGATTGGCTGGACCCCCTGGCGAGTCTGGACGTGAGGGAGCTCCTGGTGCTGAANGATCCCCTGGACGAG
ATGGTTCTCTGGCGCCAAGGNTGACGNNGTGAGACCGNCCCTGCTGACTCCTGTGCTCCTGCGCTCCCGGGCCCCCGNCCCTGTCGACTGCCGCAGACGNNGA
CGGGTGAGACGGCCTGCTGTCTGCTGTCCCATGCCCGTTGTGCCGGGCCCGTGNACCCAGCCCCCGGGACAGGTGAAAGCGACAGGGACGAGCATAAGTCACG
NGCTCTTGTCTCAGTCCCCGCCTCCGCTTCTGGAGCAGTCTTCGACTTGTCTGTGGCCGCGCCCCGTTTTGTTCTCGCA
>‘000203a-047.scf” came from CONTIG 36 at offset 0;
“E:\SEQUENCE\export\EST_db\000203a\000203a-047.scf’ (41>502)
CACGAGGACGGACCTGCTGGAGCTCCTGGCACTCCTGGACCTCAAGGTATTGCTGGACAGCGTGGTGTGGTCGGCCTGCCTGGTCAGAGAGGAGAAAGAGGCT
TCCCTGGTCTTCCTGGCCCCTCTGGTGAACCCGGCAAACAAGGTCCTTCTGGAGCAAGTGGTGAACGTGGCCCCCCTGGTCCCATGGNNCCCGCTGNATTGTC
TGGACCCCCTGGCGAGTCTGGACGTGAGGGAGCTNCTGGTGCTGAAGGATCCCCTGGACGANATTGTTCTCCTGGCGCAAAGGTGACCGTGGTGAGAACCGGC
CCTGCTGACCCTCTGTGCTCCTGCGCTCCCGTGCCCCCGNCCNTGTCGACTGCCGCAGACGTGATCGTGGTGAAACAGGCTGCTGTCTGCTGTCCATGNCCNG
TGTGCCNGGNCCCCTGNACCCAGACCCGGTGACAGGGAAAAGCAACAACG
>‘000203a-042.scf” came from CONTIG 37 at offset 0;
“E:\SEQUENCE\export\EST_db\000203a\000203a-042.scf’ (1>652)
CGGCGTCCCTCTANACTATGGATCCCCGGGCTGCAGTGGTTCTGCAGCTCTGTGGCAAGCCGCGGAGTCTGGGTTCTGATCCGCAGGATGGGGTTTGTTAAAG
TTGTCAAGAACAAGGCCTACTTCAAGAGATACCAAGTGAAATTCAGAAGAAGGCGAGAGGGCAAAACTGACTACTATGCTCGGAAACGATTGGTAATCCAAGA
TAAAAATAAGTACAACACACCTAAATACAGAATGATTGATTCGTGTAACGAACAGAGATATCATTTGTCAGATTGCTTATGCCCGTATAGAAGGAGATATGAT
AGGTTGTGCAGCTTATGCTCACGAACTCCCAAATATGGNGTGAAGGNTGGCCTGACAATTATGCTGCGCATATTGTACTGGCCTGCTGCTGCCCCGCAGCTTC
TTTIATAGGTTGGATGGACAAAATTATGAAGCNAGACGAGGNGATGGAGAGATACATGNGNAAGCATCGAGCCAACTGGGCCTCACTGTACTGNAGCAGACTG
CAAACTCTACGAGTTAAGTTTGGCCCTAGGACGCGAGAGCTGCTTTCTACAGACACGTCCTGTTATCAAAGCAAATCAGCGAGCCCGAAGCATAGGCAAAGTG
AATACGCCCCTGTGGAAAAAATCCAAAACTTTTCA
>‘000203a-044.scf” came from CONTIG 38 at offset 0;
“E:\SEQUENCE\export\EST_db\000203a\000203a-044.scf’ (1>627)
CGGCGCCCCTCTACAACTATGGATCCCCGGGCTGCAGNAATTCGGCACGAGGCAGGACAATCAAGTGTGGCAGCTGGGCTCATCGTCCCCAAACTTCACTCTG
GAGGGACATGAGAAAGGTGTGAATTGCATTGATTACTACAGGGATGGTGACAAGCCATACCTCATCTCTAGAGCAGATGACCGTCTTGTGAAAATATGGACTA
TCAGAATAAACTTGTGTACAGACACTGGAGAGGACATGCCCCAAATGTGTCTTGTGCCCAGTTCATCCTGAGCTGCCCATTTTATCACAANTTCAAAAAATGA
AACTGGCGTTTTGGCATTCAAGCACCTTCGCCTTGAGAGGACTTGATTATGAATGGAGAAGAGATGGGGGGGCCGCCGCGAGGTCCATAACGTGTTTTGGCTT
TGAGAAAGAAGCATATGTTAACTTGTCGGAGAACTGCTTGTCTGGTGCCAAGGAAAATAATGGGCCAACATCAAAATCACCAGCCACTAAACAAGGAGAGTGT
AATTAAAGAAAAGATGCCTGCATAAAAATGCAGTTGAATTACTCAATATCACAATCTAAGCGCGGTGGGGGGGGGGGGGAAATTTTTCCGCAGCCCGAAACAA
TGTGGGCCG
>‘000203a-045.scf” came from CONTIG 39 at offset 0;
“E:\SEQUENCE\export\EST_db\000203a\000203a-045.scf’ (44>624)
GCACGAGGCTTGCCTGCTGCCTGCCTGCCTGCCACTGAGGGTTCCCAGCACCATGAGGGCCTGGATCTTCTTTCTCCTTTGCCTGGCCGGGAGGGCCTTGGCA
GCCCCTCAACAGCAAGCCTTGCCTGATGAGACAGAAGTGGTGGAAGAAACCGTGGCCGAGGTGGCCGAGGTACCCGTGGGAGCCAACCCCGTCCCAGGGGAAA
TAAGAAGAATCGATGATGGTGCCGAGGAAACCGAGGGGAGAGTGGGGANCGAGAACCCCCGCCAAACCACCACTGCAACACGGCAGGNGTGTGAACTGAACGA
GAACACACCCCATGGTGTTGGCCAGACCCCACCACTGCCCTGCCCNTCGCGAAGTTGAGAGTGTGCACAACGACAACAGACTTCGATCCCTGCCATTTTTGCA
CNAGGNACATGNAGGCACCAAAAGGCCCAACTCACTGNCTACTCGGCCTGAATACATCCCCTGGTGCATCGACTGATGATCCTGGCTGCGACGCTAAACGCCG
NCACTGACAAGGAAGCACACTCGACGAAAATATGGATGAAAAACCAAATAAAGCCGGGGGGGCCTC
>‘000203a-048.scf” came from CONTIG 40 at offset 0;
“E:\SEQUENCE\export\EST_db\000203a\000203a-048.scf’ (38>559)
TTATACTCCAAGGCCTGGCAAAATCACATAATCAAGATTGAATTGTTTCAGAAATATTGGCAGGATTCTTGGACTGTGTCTACTACAGAATGAACTGTGTCCT
ATCACATTGAATAGACATGTGATTAAAGTGTTGCTTGGTAGGAAAGTCAATTGGCACGATTTTGCTTTTTTTGACCCTGTGATGTACGAGAAGTTGCGGGCAC
TTATTCTTGCTTCTCANAGTTCAGATGCTGATGCTGTTTTCTCAGCAATGGATTTGGCATTTGCAAUGACCTGTGITAAGAGAAGAGGGGAGACAGNTGAACT
ATTNCTATGTGTAATATACCAGTCACTCTCAAATGTTATGAGTATGTGCGGAATATGCTGACATAAATGTNNGTAGTGCAGACACCATACTGCATGAGAAGTC
TCTGNTGTGCTTCAAAATCATATANATTACACAGAAATTAGGTTTGTTAGCTGGNGAGNTACGGCGAGCGTCAGTCACTCTTCTGAGATAGAAAAGTGAACTT
GCGTC
>‘000203a-049.scf” came from CONTIG 41 at offset 0;
“E:\SEQUENCE\export\EST_db\000203a\000203a-049.scf’ (1>306)
GGGCGCCCTTAAATAGGATCCCCGGCCTCAGGGTGGCAAGAGGCCGTGCTATTTTTTTTTTGTAGAAGTTTGTCGCTGATGGCATCTTCAAAGCTGAACTGAA
CGAGTTTCTCACTCGGGAGCTGGCTGAAGATGGGTACTCTGGAGTTGAGGTCCGAGTTACACCAACCAGGACAGAAATCATTATCTTGGNCCACCAGACACAG
AATGTACTTGGTGAGAAGGGCCGGCGGATCCGGGAATTGACTGCTGTGGTTCAGAAGAGATTTGGCTTCCCTGAAGCAGTGTAAAGCTTATGCTGAAAA
>‘000203a-050.scf” came from CONTIG 42 at offset 0;
“E:\SEQUENCE\export\EST_db\000203a\000203a-050.scf’ (39>525)
NAATTCGGCACGAGGCTAGTCTCGAGTTTTTTTTTTTTTTTTTTTCTTTGGAAAACCAAACATGCTTTATTTCATTTTTTTCACAATTTATTTAAACATCTCA
CATATACAAAATAGGTACAATTTAATTTTTCTGCTTGTCCGAGAAACAAGACTTCTTTGGAACCATGGNAGAGGATGAAAATGAGACTGGCAAAGAACAAATG
CTGAANTTAAAGAAGAGACAANTGTGGGCAAATGATCCACTTACTTTTGTGGAATAAGATGTAAAGTACTGATGTTAAAGTCAAATGAAAAAAATACACAATA
CAGCTCAACAGCAGAGGAGTATCTCTTCTCAAATTCTCCTAGCACCATCAACATTCTTNCAGTATCTGAAATACTGTTATTAGCACCTTCGTATTTTGAACNA
AAAAACACAAATACCTCAGCTCATCTCTGGTCAGCACTCACGGTGTGGTATCACACTCACAGGAAANGTTTTGA
>‘000203a-051.scf” came from CONTIG 43 at offset 0;
“E:\SEQUENCE\export\EST_db\000203a\000203a-051.scf’ (38>406)
NAATTCGGCACGAGGATCATATAGTAAACCCAAGCCCTTGACCTCTTACAGGAGCTTTGTCTGCCCTCTTAATAACATCCGGCCTAACCATGTGATTTCACTT
TAACTCAATGACCCTGCTAATAATTGGCCTAACAACAAATATACTAACAATATACCAATGATGACGAGATGTTATCCGAGAAAGCACCTTTCCAGGGGCACAT
ACCCCAGCTGTCCAAAAAAGCCTCCGTTATGAATATTCTTTTTATTATCTCCAAGTACTATTCTTTACCGATTTTTTTGAGCTTTTACCACTCAGCCTCGCCC
CACCCCTGACCTAGCGCTGCTGACCCCCACACGCATTCACCCACTAACCCCTACAAGTCC
>‘000203a-053.scf” came from CONTIG 44 at offset 0;
“E:\SEQUENCE\export\EST_db\000203a\000203a-053.scf’ (37>515)
TGAGAGCAGCAGCCAAAAACCACGCTCGAGTGACAGTAGTATGTGAGCCGGAGGACTATGCAGCTGTAGCCTCAGAGATGCAGGATTCTGACAGCAAAGACAC
GTCCTTGGAGACAAGACGCCAGTTAGCCTTGAAGGCTTTTACTCATACAGCACAGTATGATGAAGCAATTTCAGATTACTTCAGGAAAGAGTACAGTAAAGGA
GTATCTCAGATGCCCCCTGAGTATGGAATGAANCCTCATCAGACTCCTGCCCAGCTGTATACGCTGAAGCCCAAGCTCCNTTATCACAGTCTGAATGGAGCCC
NTGATTTATAANCTGGGTGATGCTTTGAATGCCTGCAGCTGGTGAAGGAACTCNAAGAGCTTTTAGCTTNCACTGCTGCGTCTTCAAACATGTAGCCCACAGG
CTGCTGTTGGATTCACTCATGAAGAGAAACCACTCTGCATGTTATGATTGTACAAACCTCCACCGCA
>‘000203a-054.scf” came from CONTIG 45 at offset 0;
“E:\SEQUENCE\export\EST_db\000203a\000203a-054.scf’ (40>404)
CCTAAATTACTCAATAGTTTTAAAGTGTTACATATTCAAAGCCTTTTCCAGACCAGGGAGAGAGTTCTGTTAGAGTGAAGGGTAGTGTCTCTTGCGCATTCTG
TGTGTGTATTTCTAAATGCTACTGTGTGTGTTTGTGTGTGCTCCCACAGTTTATATGCAAAGACTTTGAGCAACATTTATAAAAAGTATTTTCTCTTTAGAAC
AATTCAAGAGATTTATTTTGTGGCTACCACAGNCACTGCCAGTGGATTGTTTTTTCTCTTAAATCTGAATATTGACCAAAAATTTGGTGATTTTTATGACTTT
GTTGTG7FPGGTGTTTAATTTTCTTAAAAATTTAAACTTTGGTTAAAATTCAGAAATC
>‘000203a-055.scf” came from CONTIG 46 at offset 0;
“E:\SEQUENCE\export\EST_db\000203a\000203a-055.scf’ (1>728)
GTGCCTCTCCCTATACTATGTATCCCCGCGCTGCAGTATATTTTGCATGTAGGAGAACATGTAATCTCTATCTTCCCTTAGCAGGATCAAACCTAGGGCCTTC
TGCATTGCGAGCACAGAAGCCTATCCTCTGGACCACTTGGGGGAGTCCCCGCTTTTCTTTGCATCCCAAAGAATATTATAACTAACCTAAAGAAAACCGCATT
TTCCCCTTATCGGCGCGCTTCTTTCCTTTAGTCTAAGACAATAATGTCTTATTGTCCTGGGGGGGACATAGTCAGTACGGGTTAAGCTCCTCTAACCTTTGGT
GGCATTTTTTGCCCCAAATATTGCTTTTCCAAAAACCACAGAGGCTGTTTCTTTTATTAAATTCCTTCTGCCGCCCCCCACTTGGGGGTGGGTTGGCCTCTTG
GGTCTTTTCTTAATAATAAAACAAAACGGTTTGACTGTGTCTCCCCCAGGTACTTTTTTTTCTCTCTTTCAGAGTACTGTCAACCGGACTCCAGTTTTCTCCT
GGGACGTCAAATTTTCCTACTCTCATCGCCTCTGCTGTGGCCTTTTTCCATTTCAAGAAATTCTCGACCTATTATTCACTCCTTAAAAAATATATCATGCCCC
CCCCCTCTACTTGCTTTCGCGACAGACACAACAATTTTCTTCTAAGCAGGCGAACCACAACAGAATAGGAAGATATCTATACAGAAGGAAAGAACGTATTCAT
GGCGATCT
>‘000203a-056.scf” came from CONTIG 47 at offset 0;
“E:\SEQUENCE\export\EST_db\000203a\000203a-056.scf’ (17>140)
TCAGGGATCCCCGGGCTGTCAGCTGCGTCAAGTGTTGTCATGAATGTTTTCCTGGGCCTGGCGGCGACTACAGCGGTGCTGTTCTGTCCTGTCGCGGGCTGCG
GTGACTCGCTGGCGGCTCTTC
>‘000203a-057.scf” came from CONTIG 48 at offset 0;
“E:\SEQUENCE\export\EST_db\000203a\000203a-057.scf’ (10>722)
CTGTATACTATGGATCCCCGGGCTGCAGNAATTCGGCACGAGGAGGTATGTCTGAATGTGTTTCGACTATTTACAGCCCCCTTTCCTCGCAGGGCCCCATGAG
TAAGCTGTGGCGGCGCGGGAGCACCTCTGGGGCTATGGAGGCCCCTGAGCCTGGGGAAGCCCTGGAGTTGAGTCTAGCGGGTGCCCACGGCCACGGAGTGCAC
AAGAAAAAGCACAAGAAACACATGAAGAAACACAAGATAAAACACTACCAGGATGAAGAGGCTGGGCCAACGCAGCAGTCTCCTGCCAAGCCCCAGCTCATAC
TCAAATCAAGTTGGGCGGGCAGGTCTTGGGCACCAAGAGTGTTCCTACCTTCACTGTGATCCCTGAGGTCCTCGCTCACCCCTCTCCCTTATGTNTGGGAACA
TGAAGAGTACCTGTGAAGGAGCCCCCTGACATACCGCGCTGCTGGAGAAACAAAACTGCCCCTCCCATGGGNACTGCTGNGGNTANAGCCAGAGAAGAGAATA
CGAGGGCTGAGCTTGNAAAGGGAGCGAGACAGAAACCAAAGAAACAGACGCTGCACGTGCAGAGTTGTGAAGCCGAGCACTCCGGTGCCTCTGCTGGCTGCGG
CCCTTACAAAAGGGGAGGGGGGGCGGAAGGGGGGGAGGGGCGGGAAAAAAAAAAACCCCAAGAGCGGGGGGGGGGGCGGGGGGGGGGGGGCCCCG
>‘000203a-058.scf” came from CONTIG 49 at offset 0;
“E:\SEQUENCE\export\EST_db\000203a\000203a-058.scf’ (38>620)
NAAAGACTTCATGAAGGAGAAGAAAAGGAAAAAAATCAAAGGATGACTACAGCCTCTCTTCATCTCAGCTTTGCAAAGCATGACTCAACAGAGAGATCACATT
CTTGAAGACTCTCCAGACCACAGATTGAAGATAAGTTGTTGTGCTGCACTATTTCTGYYGCTACTCGTGAGAGTCACTACCAGCCTCTCTTCTCAGGATTAAT
GAATGGGCCAGAAACCGGGGGCAGAATGACTNCAGGGGCACCCCCAACACCAGATATTGGCATAAGGAAACAGTACAATGAAAAAAGTCATACTTGGACCCTT
GTCATTCCACANAGCATGATGTACTATCTAAAACAAAAAGAAGAGCTGCTTTGGAGAAGGGTTTTAAAAGTTGTTTATCAAAANAAAGATTANAAGAGGAAAA
CTCCAGTNTATNAGATGGCATTTACTTTNAAGGCTCTGTCCCTGTGTAGGCATGAGATTGATACAGNACAGACGGGCTGCCAANCAGNTACTAATGTNTCTGC
TCTAAGAAGGGNCCTCTTGCTTCCTTGTGATATCTTGCAGAGCCGACTGAGGACAGAAACACAATAAG
>‘000203a-059.scf” came from CONTIG 50 at offset 0;
“E:\SEQUENCE\export\EST_db\000203a\000203a-059.scf’ (39>620)
CAATTCGGCACGAGGGTGAGTGACATCGTCTTTAAACCCTGCGTGGCAATCCCTGACGCACCGCCGTGATGCCCAGGGAAGACAGGGCGACCTGGAAGTCCAA
CTACTTCCTTAAGATCATCCAACTTCTGGATGATTATCCAAAATGCTTCATTGTGGGAGCAGACAACGTGGGCTCCAAGCAGATGCAGCAGATCCGCATGTCC
CTCCGCGGGAAGGCTGTGGTGCTGATGGGCAAGAACACGATGATGCGCAAGGCCATCCGAGGGCATCTGGAAACAACCCGCTCTGGAGAACTGTGCCTCACAC
CGNGGAATGTGNNGCTCGGTCACCAAGAGACCTACTGAGACAGGACAGCTGCTGCCACAGGGCCACTGCGCCCGGCTGTGCATACGCCGGGAGACCTGGCCAC
CAGACATGNCTGGGCCGAAAGACTTCTCTCAGCTTAGCACACACGATACTCAGGCACAGAACGTGAGAGGCATGATAAAAGAACAGAGGGCAGAGCACGCGAC
ACGACACCCCTTCCTGCGCCCCAAGGGGAAAGCAGATACCCAGCTAAAAAGACCGCTCCTCGGGGGG
>‘000203a-083.scf” came from CONTIG 50 at offset 7;
“E:\SEQUENCE\export\EST_db\000203a\000203a-083.scf’ (45>614)
GCACGAGGGTGACATCGTCTTTAAACCCTGCGTGGCAATCCCTGACGCACCGCCGTGATGCCCAGGGAAGACAGGGCGACCTGGAAGTCCAACTACTTCCTTA
AGATCATCCAACTTCTGGATGATTATCCAAAATGCTTCATITGTGGGAGCAGACAACGTGGGCTCCAAGCAGATGCAGCAGATCCGCATGTCCCTCCGCGGGA
AGGCTGTGGTGCTGATGGGCAAGAACACGATGATGCGCAAGGCCATCCGAGGGCATCTGGAAAACAACCCGGCTCTGGAGAAACTGTTGCCTCACATCCGGGG
GAATGTTGGCTTCGTGTTCACCAAGGAGACCTCACTGAGATCAGGGACATGCTGCTGGCCAACAGTGCCAGCTGCGCCCGGCTGTGCCATACGCCGGTGAAGC
CTGGCCAGCCAAACATGTCTGGGCCCGAGAGACCTCTCTTCAGCTTAGCACACAGAANATTCAGGCACATGAAATCTGAGAGGCACGATAGAAGAACAAGAGC
GCACGAGCAGCGTGACAGCGACACCCCTTTCTCGCGNATCACAGGTGACAGCAATT
>‘000203a-060.scf” came from CONTIG 51 at offset 0;
“E:\SEQUENCE\export\EST_db\000203a\000203a-060.scf’ (1>262)
GGGCTAACAGTCCGCGAGCCCGGCAATCCGCAGCCGGGCCCACAGGAACATGCGTCTGCTTGGGGGGGAGAGGGCCGGGCTAGAGCGAGCAAGGTGAGGGGGG
GGGGGGGGGACCTCCCGCGGATACAAGGTCACACACCCCTCCTAATGCAGAAGGCGACGGTTGCAGGAAGGGCAAAATAAGGACTCGCAAGGTGTCTAGGGGA
ACGAGTAAATGAAAGGCCACGGCGCGAGACGCGAGCGACCACCCAGGAGAACCGCG
>‘000203a-061.scf” came from CONTIG 52 at offset 0;
“E:\SEQUENCE\export\EST_db\000203a\000203a-061.scf’ (39>494)
NAATTCGGCACGAGGGTGAGTTCATTGAGTTAAATAAATACCTTTTGGAAAGGAGTTTGCCGATGCACCAAAAAAGCCTGTCTGCGCTGTAGGAATGTGTGGT
GAAGCTCAATTTCTGTTTTATGAAACCTGTTTGGGCGGGGGTCTGGGGGTTGCACAGAGAATGAGTTCTTGTATTTCGCGTCACACAGGTAGTTATGGAAATA
TGTTATTGTACTGTGTAAAGATGCCCAGCCATTTTGATTGTTTGGCTTTTTACTTTTGTACCTTTTCAAGCTTTTGCTATACATCTGGAACCCTCAACACATA
CTGTGTTGTACTTCCTTTTGTAATGATTTTTAATGGAAGTTTGCACATAACTCTTGTTATACTGTACGATAATCTTGGGGGAAAATATTTTGCATATCAAAAA
AAAAAAAAAAAAAAAACCGAGGGGGCCCGCCCCCATTCCCCTTA
>‘000203a-062.scf” came from CONTIG 53 at offset 0;
“E:\SEQUENCE\export\EST_db\000203a\000203a-062.scf’ (1>630)
CGGGCGCCGTTAACTAGGTCCCCGGCTCAGCAGACACAGTGTCGTGAAAACCACCGTTAAACCTAAGCCAAAATGGGAAAGGAGAAGACCCACATCAACATCG
TTGTCATTGGGCACGTAGATTCAGGGAAGTCTACCACGACTGGCCATCTGATCTACAAATGTGGCGGGATCGACAAGAGAACAATTGAAAAGTTCGAGAAGGA
GGCTGCCGAGATGGGAAAGGGCTCCTTCNAATATGCCTGGGTCTTGGACANACTTAAAGCTGAACGNGAGCGNGGNATCACCATTGATATCTCCCTGTGGAAA
TTTGAGACCAGCAAGTACTATGNTACCATCATTGATGCCCCAGGACACAGAGACTTCATCAAAAACATGATTACAGGCACATCCCCAGCTGACTGTGCTGTCC
TGGTCGGTGCTGCTGGGTTGGNNGAATTGAAGCCGGATCTCCAAGACGGCAGACCCGNGAGCTGCCTTTTTGCTTACACCTGGNNGNGAAAACACTATTGTGC
GNNTACAAAGGATNCACTGACACCTATACAGAGAATCAANAATGTTAGNANCACACTTATANAAATGCTCACCCGACANACATTGGCCATTTGCTGAAGGACA
AGCTAACAAGCT
>‘000203a-075.scf” came from CONTIG 53 at offset 27;
“E:\SEQUENCE\export\EST_db\000203a\000203a-075.scf’ (41>615)
CGGGACACAGGTGTCGTGAAAAGCACCGTTAAACCTAAGCCAAAATGGGAAAGGAGAAGACCCAGATCAACATCGTTGTCATTGGGCACGTAGATTCAGGGAA
GTCTACCACGACTGGCCATCTGATCTATAAATGTGGCGGGATCGACAAGAGAACAATTGAAAAGTTCGAGAAGGAGGCTGCCGAGATGGGAAAGGGCTCCTTC
AAATATGCCTGGGGTCTTGGACAAACTTAAAGCTGAACGTGAGCGTGGTATCACCATTGATATCNTCCTGTGGNNAATTGAGACCAGCAGTACTATGNTACCA
TCATTGATGCCCCCAGACACAGAGACTTCATCANAAACATGATTACAGGCACATCCCAGCTGACTGTGCTGTCCTGATCGTGTGCTGTGNTGGNNGAATTGAG
CCNGCATCTCCAAGACGGCAGACCCGGAGCTGCCCTTTTGGCTACACCTGGTGTGAAAACACTATGTTGGCGTTACAATGGATNCACTGACACCTTANCAGAG
AATCAANAAATGTAGAAGCAGACTATTAAAAATGCTCACCCGACAGACTTGGCCATTTGT
>‘000203a-063.scf” came from CONTIG 54 at offset 0;
“E:\SEQUENCE\export\EST_db\000203a\000203a-063.scf’ (10>605)
GCTCTATACTATGGATCCCCGGGCTGCAGCGTCACTTACCTCACTCGTTCGGAGTCGTATATCGGGGGAAATTGCTACATTCTGTCAGGGTCACGTGATGCAA
CCCTTCTGCTCTGGTACTGGAATGGAAAAAGCAGTGGTATTGGAGATAACCCGGGCAGTGAGACTGCCACTCCGCGGGCCATTCTGACAGGCCACGACTACGA
GATCACTTGTGCTGCTGTCTGCGCGGAGCTCGGCCTCGTGCTAAGTGGCTCCAAAGAGGGACCATGTCTCATACATTCCATGAATGGNAGACTGNNTAGGGAC
TTGNAGGNTCCANAAAACTGCCTGAAACCAAANCTCATTCANGCGTCGAGAGAGGCCATTGTGTCATTTTTATGAAAATGGGCTCTCTGCACATCATGTAACG
GAAAGCTCAGCCACATGGAACGACATACATAAGGCATCACTGACGGNATGGCAGACTGCTCACGGAGAACAGGGGGCTCAGTCTGCGGGTCGACTAACATGTC
GCTACAGTGGAGCGGATCGGCTGGCTGCTAACAAGGCGCTGTGCTCCTAGACACGTG7FICACATCACGGGACCAACACCCT
>‘000203a-064.scf” came from CONTIG 55 at offset 0;
“E:\SEQUENCE\export\EST_db\000203a\000203a-064.scf’ (44>603)
GCACGAGGCCTGGACCCCCTGGTCCCCCAGGTCCTCCCAGCGGCGGCTACGACTTGAGCTTCCTGCCCCAGCCACCTCAAGAGAAGGCTCACGATGGTGGCCG
CTACTACCGGGCTGATGATGCCAATGTGGTCCGTGACCGTGACCTCGAGGTGGACACCACCCTCAAGAGCCTGAGCCAGCAGATCGAGAACATCCGGAGCCCT
GAAGGCAGCCGCAAGAACCCCGCCCGCACCTGCCGTGACCTCAAGAGTGCCACTCTGACTGGAAGATGCGAGATACTGGATTGACCCNCACCANNGCTGCACG
TGGATGCCATTAANNGTCTCTGCACATGGAACCGGTGAGACCTGGTATACCCACTCAGCCANGTGGCCCATATAACTGTATATCACAGAACCCAGTAAAAAGC
ACGTCTGTACGGGAGACTGACGGCGATTCAGTCGATTGCGGCAGGGTCGACTGCGAGGGCATCATGATTCTGGCTGAGNCACGAGCTCAAAATACTACATGAG
ACAGGNCTATGACACAATGCACTAAAGCCGTCTCAGCTCAGATGA
>‘000203a-066.scf” came from CONTIG 56 at offset 0;
“E:\SEQUENCE\export\EST_db\000203a\000203a-066.scf’ (39>329)
GTCTCAATGTCCGTGGCGCTGAGGCAAGCTTGTGGGGGAGAAGGGTAGCGACTGTAGCTGCCGTTTCCGTTTCCAAGGTTTCGACCAGGTCGTTGAGCACTTC
CACATGGAGGCTGGCACAGGACCAAACTCGAGACACGCAACTCATAACAGTTGATGAAAAATTGGATATTACTACTATAACTGGTGTTCCAGAAGAGCATATC
AAAACTAGAAAAGCCAGATCTTTGGTCCTGCTCGNCATACATGCAGTCTGTAGTTAACAAACACAAGAATGGAGATGGAGGTTG
>‘000203a-067.scf” came from CONTIG 57 at offset 0;
“E:\SEQUENCE\export\EST_db\000203a\000203a-067.scf’ (45>669)
GCACGAGTGGCGGATGACGCCGGTGCTGCGGGAGGGCCCGGAGGCCCCGGGGGCCCCGGAATGGGAGGCCGCGGTGGCTTCCGCGGAGGCTTCGGTAGTGGCG
CCCGGGGCCGGGGTCGCGGCCGGGGTCGGGGCCGGGGCAGAAGCCGCGGAGCTCGCGGAGGGCAAGGTCGAGGACAAGGAGTGGCTCCCCGTTACCAAGCTGG
GCCGCCTGGTCAAGACATGAAGATCAAGTCTTTTGAGGAGATCTACCTTTCTCTCTGCCTATCAAGAGGCTGAGATATTGACTTTTTTCCTGGGAGCATCCTT
GAAGATGAGTTTTGAAGATTATGCCGGGCAAAACCAGACCCGGGCTGCCCAGGAACCAGTTCAAGGCGTTGTTGCTTTCGGGGATACAACGACTGGGGGCTGG
TGGCAGGCCCAAGAAAATACCCTGCCTCCGGGGGCCATCTTCTGCTAAGTGTCCACGCCCGGGCAAGAGCTTAGGGGAACANAAGACACCCCCCCGTCTGCAG
GGACGGCTGGGTCCGGCGGGCCCTACCTCCCAAGACGGCTCCTCGCCCGGCCAAACGGAGAGCGCTGACAGTCCTTTGCCGGCGCTGCCCTGGCATCCAGCCT
TGTCTCCA
>‘000203a-068.scf” came from CONTIG 58 at offset 0;
“E:\SEQUENCE\export\EST_db\000203a\000203a-068.scf’ (40>680)
TTTTTTTATGGCTTCTTCTTTCTTTATTGGACGCTTTGTAGATGTCACGCAGGTCTAAAAGTTACACCGTTAAATAATTATTTAAAAACCAACCAGGATTAAG
GCCCTGGCCCAGAGCTCCAAACCAGAAGCAGAAAGGAATGGTGGCGGTGGGCTGGGGGGGTATTCCTCCAACATCACCAAAACCCAGAGAACGAGGATCCTAA
GCTTTTCACAGGCCAACCCGGGCACGGGCCTGCAGGCTGACCCTCGGAGGCCTCTGGCTGCATCACTATCAGATCAAAACCAGCGAGGAGCTGCCGGGAACAG
CCAGCCGAGTCCAGACATGGACACAGTAGCTGGATGGACACGAGACGGACAGGTCCTGTCCAGCTGTGGACAGGATTCAGATGCAAGCTAGGCAGTGGGGGCA
GGGGCTGGGGAGCAGAATGAAGCATGCAGGAGGGGCCCCGGGGCCTGGCTCANCCACCGGCCGCCGAGCCTCACCGTGTGGGNTCGCTGGGGNCTGGGCTCCC
GCCCACACTGGACTTGAGGCTCTGNAGCGAGAGTTCCAGCCATTGTGATGTTGCTCTCACAACAAATNCTTGCCACTGGGAGAATTGAGATGGTGACAAACTA
CGCCCAAGCATGTGGATGCCAGC
>‘000203a-069.scf” came from CONTIG 59 at offset 0;
“E:\SEQUENCE\export\EST_db\000203a\000203a-069.scf’ (12>643)
CTCTATACTAGGGATCCCCGGGCTGCAGTAATTCGGCACGAGGCTCGGTTTTTTTTTTTTTTTTGCTCTAATTAAAATTTTTATTGAAATCTCTCAAACGTTA
CCAAGAAATAGTTTTTGCAAAAGGGAGGGAAGGGAAAAAACAAACAACAACAACAAAAAAGAGCTAACAAGCAAATTCAACATGGGAGCTCCCTCTGCTGGTC
TGCAGTAGGTTGATATGTTACAAACACATTCCCAGAGACAAATCTATTTGCTGGAGAAGGGACAAAAAACAGGTGTGTGGGCTTTGCCTCNAGAGAGAACACT
GGCATGCAGNGAGCGGGGTAGTGAAAGCAGAGGAGCAGCGCAGCGCTAAGTCGCTGGTACAGAAGTACGGGCCACACTCAGGCTATGNGTAAAGGCAGCCTGT
GACTCTATGTCTCTGCATGACTGAGACAGGTGGCAAGNAACTGGGTTGGAGCCTACTATTGTCTTGCTCTCGGGTTGCTACAACCTATGTAGGTTGCGATTAG
ACGACGAGCCTCAAGATTTGCGCTTTGTTGACATGCAATGCAACTAGCTGTATTACTTTAAAACTTTACCTATGTGAAAAATAATCCGTGATCAAGGAAGGCA
AAAAAACTCTTT
>‘000203a-070.scf” came from CONTIG 60 at offset 0;
“E:\SEQUENCE\export\EST_db\000203a\000203a-070.scf’ (19>728)
CTACGGCATCCCCCGGGCTGCAGGTCGAGTTTTTTTTTTTTTTTTTTTTAGTTAATTACTTTATTACATTTTAGTGCTTTCTTAAAATAAATATAATAATATA
ATTATCAAACATACAGTGAGAAGTAAAGCACACGTGTGAACGGCATGTCACAGGAGTTCACTCAGGACTGTTTCAACACTCAGCACTGGAGAAACCGCACAGG
CCTACCTATGTACAGACGACCCAACCGCCAGGGCGAGGCCACCGCGTCCCCTCCGTCCTCATGGACACGGCCACTCCCCCTTGCGTTGAAATGACCAGTTTGC
ATGTTTTAACTTTTCTCTCCGTTGAGCTTCAGTTTTTTTTTTTTCTTTTGCAGTTTTGAAAAAATTCAAGTAACACTCCCAAGAAAAAAAAAGTGCAAACTAA
TAAGGGACTCAGAGTCCGGCGCCGGTCAGGGGCAGCGCACAGCGGGGGGGCAGCCGGCCGAGTCTGTCCCGGAACACGGGGCGCAGGACCCCGCCCACTCGAG
GAGGGGGGGACGGAGGCCGGCCTGGGTCCAAAGCGCCAGACCTTTGTTGAAAGCAGCACAGCCCGCGAACGAACGCAGTCCGCGGCCGACTTCGGACCAAGGA
AGGAGAGGGGGAAAAATAAAAGTATTAGGATCCTTTTATAAAATATAAAATTTTCAAATTTTATAAAGGGCGCCCCCGGGGGGGGGGGCCCC
>‘000203a-071.scf” came from CONTIG 61 at offset 0;
“E:\SEQUENCE\export\EST_db\000203a\000203a-071.scf’ (38>713)
TTGACATCCTCCATTTCGCTGTGCTGAGTTAGTACAGGGAAGACGGGGAAGTGAAATGCCAACAGTAGTAGATGGGGGACTTCCCTGGTAGTCCAGTTGTTGA
GACTTCGCCTTCCAGTGCTGGGGGGTGCAGGTTCAGTCCCTGATGTGGGAGCTAAGATCCCTCATACCTCATGGCCAAAAAACCAGAATGTAAACAACAGATA
CAATATJTGTAACATATTCAATAAAGAATTAAAAAAAAATTTTTTTATAAAATATATTAAAAAAAAACCTGACAGTTTCACAGAGAGGGCTGTGATAGGATTG
CTCATGAGGAAATCCAAGGAATAGAAGTTTTTGATAGAGGATGATGGGAAGTGTGTTAGCAGAAGTAGGACTGCCTACTGTCCATCTGGACCATGNAAAGCAC
ANATTCATCCCGCACAGCTTGGAGAGAATGTCTCACTAAGAGCTCATGCCTTGTATTCCTCCCACATTATTTGTATTGTTGGTTTTATATACGTTTTTTTGCT
GATATTACCATATTAGGTTTGTGTGATGATTGTAGGACATTCTATGTAGAGAAAGATAAACTTAAAAAAGAAGCCCTTTTTCTTTTTTTTNTTAAATAATGCA
TCTTAAGTGAGTCACCCTTTCGGCAAAAAAGAGAACACTTTACTTATTACTATTTCTT
>‘000203a-072.scf” came from CONTIG 62 at offset 0;
“E:\SEQUENCE\export\EST_db\000203a\000203a-072.scf’ (38>334)
CTGGTGCTTTCGGCCTCGCTGCGGTGCCGACAGTCCGTTCATGCCTCGCGTTTGAGGGCAGGGGGTGGCTCAGCGGCTGGCTCGCAGCTTTCTCCGCTGGCTG
AGGCCCGCCACAGCCGACATGGGCTGTTTCTGCGCGGTTCCGGAAGAATTCTATTGCGAAGATTTGCTTCTGAATGAATCCAAGTTAACTCTCACCACCCANC
AAACAGGCATCAGAAAATCACGAAAGGGCTCATTGTCCTTGAGCACCGTATCCGCCACTCAACCCTGGGGAGGTGAGTATTTTTGGCTTGC
>‘000203a-073.scf” came from CONTIG 63 at offset 0;
“E:\SEQUENCE\export\EST_db\000203a\000203a-073.scf’ (1>639)
CGGGGCGCCCTCTAAACTATGGATCCCCGGGCTGCAGGCCCAGTTTCCCTCCCAGAACATTCTTGGGACCAGCCACCTTTCCCCAGGTGTGTGCTGCCCACTG
CCACCCAGAGGTGGGATGGCAGGTTCCAGGTTCCCTCTTGGATCCCAGGCTTCCCCTGACATCAGCACCATTCAGTGGTTTTCCTGGCTCCATCGCTATCGCT
TCATGCTGAATGGACAGGACTGTTGACCTGTCTCAAGAAGCCCATAAGATTTGAGCAGAAACGNTGACTTCCTTGTACCTCTGCCAGAGCAGCTCCCTFFCTC
CCCCCCAGAATTTCACCAGATCTCTTCATGCTGCTCTTNATTTNCGTACTTGAAGTGGNTCTGCCGACAAACAGCAGACTLTTGTGTGTCCCACTCTGACTTG
CAGGACGGNAGNTTCTTTCTCTTAAGTTGATCGCTTCTTGCTGTGACTGNCCCGCTGGNGAAAATGGTTTTGCCTCGTTTTAAGTGAACAGGAGACTAGATGC
TGTACTAAACAGATGGAACCGAGAGAAACACTACCATCAGTAAAGACCACCACCCAACACTCATAAACGCTAACAAATCTGCTGAAAGATGTAGCTGGAAAAA
AAAAACGGGGGCCGGCCACCTTA
>‘000203a-074.scf” came from CONTIG 64 at offset 0;
“E:\SEQUENCE\export\EST_db\000203a\000203a-074.scf’ (1>580)
GCGGGCGCGCTCTAAACATGGATCCCCGGGCTGCAGGTTCCTGTGTCGTCTTGGAGGTGACTCGGCGTGATTGAATTTGCGGCATCTTCGCATTCACTCACAG
GTCAAAATGCAGATCTTCGTGAAAACCCTGACCGGCAAGACCATCACCCTGGAGGTGGAGCCCAGTGACACCATCGAGAACGTGAAGGCCAAGAATCCAGATA
AGGAAGGCATTCCCCCTGACCAGCAGAGGCTCATCTTTGCCGGCAAGCAGCTGGAAGATGGCCGCACTCTNTCTGATTACAACATNCAAAAGAGTCGACCCTG
CACCTGGNCCTNCGTCTGAGGGGGGNATGCANAATTTCGGAAACCCNNTGACGCAGACATCACCTGGAAGGGAGCCCANGACACCACGANAACGGAAGCCNAA
ATCAGATAGAGGCATNNCCCCGACACAAGCTCATCTTGCGCAGCACTGGAGAGGCGCCTCTTTGATACACANCAAAAGGCGACTGCCCGGCCTCGCGAGGGGG
AGCAATCTCGAAACCGACGCAGACATACTGAGGGGGCAGCACACCAAAGAAGCAAACAAAAAAGA
>‘000203a-076.scf” came from CONTIG 65 at offset 0;
“E:\SEQUENCE\export\EST_db\000203a\000203a-076.scf’ (45>598)
GCACGAGGCCTTCATCCAGCACCTTCCCCTGAGTGAGCGCATCCGGGGGACCGTCGGACCAAAGAGCAAGGCAGAGTGTGAGATTCTAATGATGGTGGGCCTG
CCCGCTGCTGGCAAAACCACGTGGGCCATCAAACATGCAGCCTCCAACCCCTCCAAGAAGTACAACATCCTGNGTACCAATGCCATCATGGATAAGATGCGGG
TAATGGGCCTACGCCGTCAGCGAAACTACGCCGGCCGCTGGGACGTCCTGATCCCAGCAGCCACTCAGTGCCTCAACCGTCTCATCCAGATTTGCTGCCCGCA
AGAAGCGCAACTATATCCTANATCAGACAAATGTTTATGGGTCAGCCCAGAGACGAAAAATGAGACCATTTGAAGGCTTTCAGCGCAAAGTATTGTATTTGTN
CCNACTGATGAGACTGNAAGACGAACAATAAGCGACTGACGAGAAGGAAGGAGTCCNANACACGCGTCTTAAAATGAAAGCACTTCACGTGCCGATGTGGGAC
TTCTGGAGAGNGCTGTCATGAGCTGCAGGAAAGGAGCGAC
>‘000203a-077.scf” came from CONTIG 66 at offset 0;
“E:\SEQUENCE\export\EST_db\000203a\000203a-077.scf’ (46>306)
CACGAGGGGAATCTTGTCCTTCCAGGTCCGCCAGTTTAAGCGCCTTTATGAACATATTTAAAAATGACAAGTACCTTGTGGGCCAGCGCCTCGTGAACTATGA
ACGGAAATCCGGCAAACAAGGCACATCACCACCACCTCCACAGTCGTCCCAAGAATAAAGTGGTTGTCTCCACTACCTTGGCCTTCCCCTTGCCTTCACGTGT
CCTTTTTTGTGGACTTCTCTCTCTGGAGATTTCCCCAGTGATCTCTCAGCGTTGTT
>‘000203a-078.scf” came from CONTIG 67 at offset 0;
“E:\SEQUENCE\export\EST_db\000203a\000203a-078.scf’ (39>281)
CTAGTCTGAGTTTTTTTTTTTTTTTTTTTTTTTTTTTTTCCTGTACTACTATCACTATTCTCAGGTGGGTTTTTGAGAATGAATGTGCAGAGTTTATGATGTG
TGTCAAGCATGCCTCGATAGCCACAGGCTTTACAAAAATTACCTATTGTTGCTTCTTTGGATTGACATGCAAATCTGTTTCATGATTCTCACACTCACGACAG
AAAACAAATTTTTTTATGAATCCATCCACCATGCTT
>‘000203a-068.scf” came from CONTIG 68 at offset 0;
“E:\SEQUENCE\export\EST_db\000203a\000203a-079.scf’ (16>24)
ACAGGGATC
>‘000203a-080.scf” came from CONTIG 69 at offset 0;
“E:\SEQUENCE\export\EST_db\000203a\000203a-080.scf’ (16>24)
NAATTCGGCACGAGGCAAGCGCCTGCTGGAGCCCCCGTGCTCCTTGCACTTGAACTCTATGGGGTTTFGGTGGGCAGAGGCTCAGGAGTCCCCTGGATTTCCC
CAGCTGGTATCCTGGGACGTGGTAAGCTTGGGGCTGGGGTAGCATGGGATCCCCCGAGGACCCANATTCTGGTACTNAGGGCAAGGNGAGGNGAACCCGNACC
TCANCCGTCCCCCAGTCTACAGCCTGAGCCCAGTGTGCTCCCAGCTCCCCANTCCNCATGAAGCCTGCCGGNGGCTGGCAGNAGGGNTTAGAGGNNCTGGCCT
TCGATTCCTTTTCTGTCGCGCTGCTTTCACCCGCTTCCTGCAGCTTTGCTCTGGCCTGATGATCGTGCTTTGTTCTCTGTACTGTTAACTGAGCATGCCACAT
TTGTTGAAATGTTGTTCAAGTGTAAGCAAGGAGAGGTCCAATTGTGATGGGGATGGAGGCATGGACTCTGCTTCTATCCTTCTACTTATGTGAAATGTTGCTT
CTGCTGTTGGATTATTATACAGGGCAACCTATACAGCGAAAAAAAAAGGCAAAAAATTCTCTACCACGAGA
>‘000203a-081.scf” came from CONTIG 70 at offset 0;
“E:\SEQUENCE\export\EST_db\000203a\000203a-081.scf’ (41>563)
CTCCAGTTACCTCTGCCAGTACCGCTGTGTCAACGAGCCGGGCCGCTTCTCCTGCCACTGTCCACAGGGCTATCAGCTGCTGGCCACGCGCCTGTGCCAAGAC
ATTGACGAGTGTGAGTCGGGTGCGCACCAGTGCTCTGAGGCCCAGACTTGTGTCAACTTCCACGGNGGCTACCGCTGTGTGGACACCAACCGCTGTGTGGAGC
CTTACGTCCNAGTGTCCGACAATCGCTGTCTCTGTCCGGCCTCAACCCCCTGTGCCGGGAGCAGCCCTCATCATCGTGCACCGTATATGAGCATCACCTCGAG
CGGAGCGTACCGCGGACGTNGTTNCAATCAANCANCNNTCGTCTACCTGTGCTACATGCTTTCAATCGTGCTGTAACTCGCAGGAACTCTACATAGGAATCAC
ATGCACGCTGCTGTCTCGCTCGGCTGGACGGCCCCGGATACGGCTGACTGAGAGTCACTTACTCTCTGATACGGCACTCTTTTGAATACGCTTGGGGCTACTT
TGGGGGGG
>‘000203a-082.scf” came from CONTIG 71 at offset 0;
“E:\SEQUENCE\export\EST_db\000203a\000203a-082.scf’ (48>533)
GCACGAGGGCCTGCTGCAGCCCGGCTGCCAGCTGGAGTCCCTGTGGGTGAAGTCCTGCGGGTTTACGGCCGCCTGCTGCCAGCACTTCAGCTCTATGCTGACC
CAGAACAAGCATCTCTTGGAGCTGCAGCTGAGCAGCAACCCGGTGGGCGACGCGGGCGTCCACGTGCTGTGCCAGGGCCTGGGGCCAGCCGGCACTGTGCTGC
GGGTGCTCTGGGTGGGCGACTGTGAGCTGACGAACAGCAGCTGTGGCGGCCTGGCCCTCACTCTGCTGGCCCAGGCCCACCTGCGGNAGCTGGACCTGANNCA
TACGGNCTGGGCGACCCCCGCGTCTGCAGCTGCTGGGGCAGCTGGAGCACCCGCTGCAGCTGGAGCACTGTCCTGTCGACTCTATGGACCGAGCATGGACGAC
GCTGCGGCTGTGGAGAAAGCAGCTGGNCTGCGATCTTTCTGACCCGTCCCCAGNGCGTNATGAAAAGTNCATCA
>‘000203a-084.scf” came from CONTIG 72 at offset 0;
“E:\SEQUENCE\export\EST_db\000203a\000203a-084.scf’ (47>388)
GCACGAGGCACAGTAGCATCACTTCAGAAAGGAGCCAGACTTATTCTCAAAGAACTATGTTCACACTTTTCAGCAGAAATAGCGATGGTTGTAACATATGTAT
CCCCTCCCTCGGATTTGAAGGCACAATCTACAGTGTTTCTTCGCTTCTTTTCTGATCTGGGGCATGAAAAACCAAGATTGAGATTTGAACTATGAGTCTCCTG
CATGGCAACATAATGTGTGTCACCGTCAGGCCAAACAGCCAGCCCTGAACGGTGGNTTTATTACTTGTGTATTTGTGTTGGATGATAAACACTCATCATCTCT
CCTGTAGTCCCTGCTCATTTCACTTAACCCTAN
>‘000203a-085.scf” came from CONTIG 73 at offset 0;
“E:\SEQUENCE\export\EST_db\000203a\000203a-085.scf’ (9>658)
CGCTCTATACTAGGGATCCCCGGGCTGCAGAATTGGCACGAGGGGAGCTCCGCATCCACACCGGCCAGCCCAGATCCCGAGGTCTGACAGCGCCCGGCCCAGA
TCCACAAGCCTGCCAGGAGCCAGCCGAGAGCCAGCCGGCCGCGCGCTCCTACCCCAGCAGTCTCTGTCCTTCGGCCTGAGCCCCGCGTCCTTCCCGGGACCTC
TGCCCCTCGGGCAGTGCTGCCACCCTGCCGGCCATGGAGACCCCGTCCCAGCGGCGCGCCACCCGCAGCGGNGCGCAGGCCAGCTCCACCCCGCTGCCACCCA
CCCGCATCACCCGGCTGCAGGAGAAGAAGACCTACAGGAGCTCAATGACCGNCTGGCTGTCTACATCGACCGTGTGCGGGCGCTGGAAACGAAATGCAGTCTG
CGCCTCGCACACTGATCTGAGAGGGGGCAGCCGGAGGGTCTGGCTTAAGCCCGCTCCAGCCGACTGGGGAGCCGCCAGACCTGACCGTGGNCAGACGCGCCGC
GCGCGGACGACAAGGGAAGAGTCAGGACCAGCACGCATCAAAGAGGAACGAGGCCCAGCCGCTAGACGAGGCGTCACCAGAGCGGCGGCTGTTAGAAGCCGGG
GGACGGACGGGGAGGCATGGGCCCGGGGCAAC
>‘000203a-086.scf” came from CONTIG 74 at offset 0;
“E:\SEQUENCE\export\EST_db\000203a\000203a-086.scf’ (48>633)
GCACGAGGATGAATTTTCACTGGCCCTTCGGCATCTGGTTGTGCAAAGCCAATTCCTPCATTGCCCAGTTGAACATGTTTGCCAGTGTCTTCCTGATGGTGAT
AAGCCTGGACCGCTATATCTACTTGATCCACCCGGTCTTATCTCATCGGTACCGTACCCTCAGGAACTCTCTGATTGTTATTATAGTTGTTGGCTTTTGGCTT
CACTAATGGGTGGGCCAGCTCTGTACTTCCGGGACACTCTGGAGTTGAATAACCACACTCTTTGCTATAACAACTTCCATGAGCATGATGTGGACCTCAGGTT
GNTGAGGCATCATGTTCTGACCTGGGAGAAAGTTATTGTTGGGTACCCTCTCCCTCTGCTAACAAGAGCATTTGCTACTTGGCCTCATCTCAAGAGAAGAACG
AGCACCTGTACTCAGAAGCCTCCTGACCACCCGGCGNGGNCATGCCTTNCGATTGCTGAATCCTATCACTGTTACATTGGAACCACGACCACACATACTATTC
ACCAAGCTACAGCACACCCCTTCACGCCGNGTTCTCAAATGCTGACCCCCCTTACCCGATATAAAAG
>‘000203a-087.scf” came from CONTIG 75 at offset 0;
“E:\SEQUENCE\export\EST_db\000203a\000203a-087.scf’ (46>645)
GCACGAGGATTTAATATTGTGGAGGGTGGGGCTTCCAGGTGAATACAGTTGCTGGTTGCTGAGCCATGCCCAACTCTTTGCAACCCCATGGACTGCAGACCGC
CAGGCTCCTCTGTCCATGGAATTGTCCAGGCAAGAATACTAGAGTGTGITGCCACTCTCTTCTCCAGGGTATCTTCCGAATATAGGGATCAAACCTGGATCCC
CTGGATTGCAGGCAGATTCTTTATCCTCTGAGCCACCAGGGAAGCTCCTAGTCACCCTAAAACCTCCAAATTCTTAAAAAAATTACCCTATCTACTTCCACCC
CAGTCTTTCTCTCTTCTTCTTTTGGTGTCTTGATTTTTGCTTTTGGCTCTGCCACTGCATCACATCACCTCTTCCAGCCTGACTATGAGTCGCCTCAGACTCA
GAGCAGTTCACTCACGAATCTTGGCTTGACCACATACTCTCGNACTTGGCTCTGACTGCTTTTTTTATTGTTATTCGACATCTCCACCCGCGAGATCTCTTTG
GACAGCCTTGTATAACATCTGTTATACGTTTTGTACGCTATTGGGAAAAATAATTAAAAGGGGCTCCCCCAAAAAATTACGCAA
>‘000203a-088.scf” came from CONTIG 76 at offset 0;
“E:\SEQUENCE\export\EST_db\000203a\000203a-088.scf’ (19>21)
TAT
>‘000203a-089.scf” came from CONTIG 77 at offset 0;
“E:\SEQUENCE\export\EST_db\000203a\000203a-089.scf’ (1>428)
AGGTGGCGGCGCTCTTATTATGGATCCCCCGGGCTGCAGAATTCGCACGAGGGAGGCCTTTCGGCCGCAGCCATGGCGCCCAGCCGGAATGGCATGATCCTGA
AGCCCCACTTCCACAAGGACTGGCAGCGGCGCGTGGCCACGTGGTTCAACCAGCCGGCTCGCAAGATCCGTAGACGCAAGGCCCGGCAGGCCAAGGCGCGCCG
CATTGCCCCACGCCCCGCGTCCGGTCCTCTCCGGCCGGTGGTGAGATGCCCGACGGGTCAGTACCACACGAAGGTTCGTGCCGGCAGGGGCTTCAGCCTGGAG
GAGCTAAGGGTGGCCGGCATCCACAAGAAGGTGCCCGGACCATTGNNGATCTCGTGGACCCGNAGCGCGGANCAAGTGCACGGAGTCCCTGCAGGCCACGTGC
AGCGCTCAAGGAGTAN
>‘000203a-090.scf” came from CONTIG 78 at offset 0;
“E:\SEQUENCE\export\EST_db\000203a\000203a-090.scf’ (42>591)
NAATTCGGCACGAGGGGAAGTGTATAATTTCCTGGCCACTGCAGGTGCCAAGTACGGCGTGGGCTTCTGGAGGCCTGGCTCTGGAATCATTCACCAGATCATT
CTGGAAAACTATGCGTACCCTGGGGTTCTTCTGATTGGCACTGATTCCCACACCCCTAATGGCGGTGGCCTGAGAGGCATCTGCATTGTAGTCGGAGGTGCTG
ATGCCGGGNACGTCATGACTGGGATCCCCTGGGAGTTGAAAGGGCCCCAGGTGATTTGGGCGTGAAGCTGACAGGCTCCCTCTCTGGCTGGACCTCACCTAAG
ATGTGATCCTGAAGGTGCGGGTATCCTCACAGTGAAAGGTGGCACGGGCGCCATCGGGNAGTACCACGGGCCTGGAGTAACTCCATCTCTGCCCCGCATGCGA
CCTCTGCACATGGTGCAGAATCGGCCACACTTGTGTTCCCTACACACAGAGAANAATACTGACAGACGGCGGCAATATGCACCTGTGAGATTAAGATACTGTA
CTGCTTGCTGCCTTTACAATTATATTACCTAGGCGA
>‘000203a-091.scf” came from CONTIG 79 at offset 0;
“E:\SEQUENCE\export\EST_db\000203a\000203a-091.scf’ (41>338)
TAATTCGGCACGAGGCCCCTTTCATCACCAACCCTGGGTATGACACTGGAAACGGTATTCATCTTCCCGGCACTTCTGGGCAGCAGCCCAGTCTTGGGCAACA
AATGATCTTTGAGGAACATGGTTTTAGGCGAACCACACCGCCCACCACGGCCACCCNCGTAAGGCATAAGCCAAGACCGTATCCGCCGAATGTAAATGAGGAG
ATCCAAATTCATGTCCCCAGAGGAGACGTAGACCATCATCTCTACCCTCACGTTGTGGGACTCAATCCAAATGCTTCTACAGGCCAAGA
>‘000203a-092.scf” came from CONTIG 80 at offset 0;
“E:\SEQUENCE\export\EST_db\000203a\000203a-092.scf’ (47>391)
GCACGAGGCAGCCCGAGGACAGCCAGCAGGACCTGCCTGGGGAGCGCCACGCCCTCCTGGAGGAAGAGAACCGGGTGTGGCACCTGGTGCGGCCCACGGACGA
GGTGGACGAAGGCAAGTCCAAGTGCGGCAGCGTGAGGAGAAGGAGCGTACCAAGGCCATCACCGAGATCTACCTGACCCGCCTGCTGTCCGTCAAGGGCACGC
TGCAGCAGTTCGTGGACAACTTCTTTCANNAGCGTGCTGCGCCCGGGAAACGCGTGCCACCGGGCGTCAAGTACTTCTTCGATTTTNCTGNACGAGCAGCAGA
AAAGCATGACATTANAGATGNANGACACCNATTNC
>‘000203a-093.scf” came from CONTIG 81 at offset 0;
“E:\SEQUENCE\export\EST_db\000203a\000203a-093.scf’ (44>356)
CAAAAACCAGAAGTGACGGGAGGTGCTGCGCTCCCCTGCGTCGTGGCAAAGTCAGCTGGCCTCTTGTGTGTGCGTGTGTGCGTGTGAGGAGCCGAGTGTGGGT
GTGTGGCGGGCGTGGGAGCAGCTTTCTCACATAGTGCCTTATACACGCTCTAAAGAAACCAGTCTTACATGTTAAGAACAACCAGTGTTACATTTTCTACACT
ACCTTCATTTCAGTAGCTTTGATGACCAGTTTTGCAGTTCATGGAGGAAATCATGGNNGCGTCCCAAGGGGCTCCCCATGCCCGAGAGCCGACTGGTCNTGTG
ACG
>‘000203a-094.scf” came from CONTIG 82 at offset 0;
“E:\SEQUENCE\export\EST_db\000203a\000203a-094.scf’ (40>373)
GGGTTTTTTTTTTTTTTTTTTTTGTAATAAATAAAAAGTTTATTAACAAGGAATGCACTTTTCCAGCCACAAGTGTCTTCAAAAATTAACAAAACAAAAAAAA
TATATATATGGCCATAGTTCACAGTTAAGCAGCCAAAAGCTGCTCCAATTATAGCCTTTAAACAACATGTGAGCATCCTCCCTTTCCCTCCCCTTCAGTAAGT
ATATTCACAGCTTCAAGTCCTCTGTCCGAAGCACTCTCCACAGAGAGAAGTTAAGAGTCAATGCACCTTTCTGCAAAATTGTCTGAAAAGCTTTANNAAACAG
TACGTCAAGGAAACTGTTCGGNTC
>‘000203a-095.scf” came from CONTIG 83 at offset 0;
“E:\SEQUENCE\export\EST_db\000203a\000203a-095.scf’ (42>489)
CGACAGCCTAGAGGGCTTCGTGCTGTGTCACTCCATCGCTGGGGGAACAGGCTCTGGCCTGGGCTCCTACCTTAGAACGGCTCAACGACAGGTACCCCAAGAA
GCTGGTGCAGACATACTCAGTGTTTCCCAACCAGGATGAGATGAGCGATGTGGTGGTCCAGCCCTACAACTCACTGCTCACGCTCTAGAGGCTGACCCANAAC
GCCGACTGTGTGGTGGTGCTGGACAACACTGCCCTGAACCGGATCGCCACAGACCGCCTGCACATCCAGAATCCCTCAITCTCCCANATCAACCAGCTGGTGT
CCACCATCATGTCAGCCAGCACCACACCCTGCGCTACCCCGGCTACATGAAACACGACCTCATCGGCCTCATCGCCTCGCTTATTCCACGCCACGCTNCACTT
TNCTCTGACTGTTTCACCCCCTCCACAGNACAGCG
>‘000203a-096.scf” came from CONTIG 84 at offset 0;
“E:\SEQUENCE\export\EST_db\000203a\000203a-096.scf’ (43>460)
CATCAGGCTCGAGGGCTCTGTTGTGCGGACTGCTCCCCCTGGACCCTCTGGTTTCTCTGGGCCCTCTGACCTCTTTGATCCTGCTGGTAAAGAAGGGCTTCGT
GGGCCTCGTGGGGACCAAGGTCCAGTTGGTCGAAGTGGAGAGACAGGTGCCTCTGGCCCTCCTGGCTTTGTTGGTGAGAAGGGTCCCTCTGGAGAGCCTGGTA
CTGCTGGGCCCTCTGGGACCCCAGCCCACAAGGCCTTTTTGTNGCTCCTGTTTTTTCTGGGTCTCCCAGCTCTACAGTGAGCGCGACTACACGTGTCGTGATC
TGTGGAGGGGTTGACACCTCTTTTCCTCGTTACATATAAAAATGTAAACCTGCCTAATAACTGGACATATGACCTGATACTCACTTATATTTTTTCTGGCTTT
CHAACAAA
>‘990729A-001.scf” came from CONTIG 1 at offset 0;
“E:\SEQUENCE\export\EST_db\990729a\990729A-001.scf’ (56>557)
GCACGAGGGGTGGTTTTGCTGTGTTAGCAGCAATGTCAATACAAGGTTCTGCAAATTTACAAACCCAATGGAATTGTTGGGGGAATTCAACAATTTGCCACAA
GAAGAACTTATTGAATGGATTAAATATAATACTAAACCGGATGCAGGGTTTGCGGGTGCCATGCCCACAATGGCAAGTGTTAAACCTCTCCGCACTCGGGCCG
TTGTGAATCATCCACATTATGAAGATGCGGGTTTAAGAGCCAGAACAAAATAGTATTTCGATGATAAACGGAAGCAGTGAGAAAGGAGGAGACTGTAAAGTAC
AGTGAATTTTCATTTGGAGAATGGGGGTAAAAAATACAAGCGGATGCAGAGCGGAATTGGGATGGGAAACCTGTATGCGGAAATCCCTTTCAACCTTTAAGAG
GCAAACCCCTCCCCGATTCCAACAAGGACAAAACTAAAAATAGAGGACGCGCTCAAAAACCCAAAGGGTTCTTTGTTTAACTGCTGTT
>‘990729A-008.scf” came from CONTIG 2 at offset 0;
“E:\SEQUENCE\export\EST_db\990729a\990729A-08.scf’ (315>902)
GAGGTTGGGGTGTGGTGTTGGGTGGGTAGGTGGTTGTGTTATGTGTGTTGTGGTGTGGTGTTGTTGTTGGTGGGTGTTGGTTGTGGTGTGAGCTTGTGGTTGT
GTCGGTGTTGTGTGGTGTTCTGGTCTGTGTTGTCGTCTTGTTGTTTCTTTGTTTTGTTTTTCTTTGTTCGCTGTTTGTGGTCGTCTGTATTCTCTTTCTCTGA
TGTTTGTTTCGTTCGTTTTCTTATTTCTTTATGTCTTATCTTTGTAATCTTTTTTTATTTTTTTCATTTTTATATTCTATTCCTTATATATATGTTACTCTTC
TTTTCTTTCCTTTTTTTTTTTTCTTTGTTTTTTTGTTTCTTTATATCTATATCTTAGCTTCTTCTATTTATATTCTTACGTACTACTACATCTTTTCCTTTCT
CTTATATAATTTTTTTCTATTCACTTTTTTGTTTATCTCTTCACTTTTCTTTTTCTCTCCCCCCCATTTTATTTTATTTTCTATTTTCTTCTCTTCTCATTCT
TGTTGTCGTATCTGCTCTATCTTTTCTGCATTTTCTCTCTTTTTTTTAATAATTTATTTAAT
>‘990729A-028.scf” came from CONTIG 2 at offset 149;
“E:\SEQUENCE\export\EST_db\990729a\990729A-028.scf’ (291>388)
GGTTGTTTTGTTTTTTGTTTTTTTTTTTGTTCTTTTTTTTTTTTTTTTTTTATTATTTATTGTATTTTTTCTTTTTTTTTTTTTTATTTTTTTATTT
>‘990729A-012.scf” came from CONTIG 2 at offset 234;
“E:\SEQUENCE\export\EST_db\990729a\990729A-012.scf’ (576>659)
TTATATTTCTTTTATTCATCTTTTTTTATTATTTCATTTTTATTATATACTTTTCATTTTTTATTTTATTTCTTTTAATCCTCTT
>‘990729A-002.scf” came from CONTIG 2 at offset 430;
“E:\SEQUENCE\export\EST_db\990729a\990729A-002.scf’ (249>314)
ATTCTATTTCTTTTTTATATCTCTTCCTTCCTTTGTGTTTTGTGTCTCTTTTTTTTTCTCTTTGGT
>‘990729A-003.scf” came from CONTIG 3 at offset 0;
“E:\SEQUENCE\export\EST_db\990729a\990729A-003.scf’ (48>272)
TTGGAGGAACGAGGGGGGGGCTGATTCCGCAGGACGCCGCCGTCGGGGCCTTCGTAUGGTGGTGGCGAGAGGGCCGGCCGCCGAGGACAGGCCGTGGCGGGTC
GAGGCACTCCCCAGGAGAGCAACATTCATAGGGTGGGTTGGATAGACGGGGTACCCGGCCCTGACCGATATACATGGCCGTTGTGGGACATTATTTCACTGTT
GGAGGGCCCTTCCA
>‘990729A-004.scf” came from CONTIG 4 at offset 0;
“E:\SEQUENCE\export\EST_db\990729a\990729A-004.scf’ (62>70)
TTTTTTTTT
>‘990729A-005.scf” came from CONTIG 5 at offset 0;
“E:\SEQUENCE\export\EST_db\990729a\990729A-005.scf’ (51>589)
CTGCCAAGGAGGGCAAGACATACAGGTGGTAAGGTGATGCCCGCTGTACCTTCTTCACCAAGGTCCGGAGATGACAGATACCACTCCAAGAGCGCACACAGGA
GGGCCAAAGCCCCACAAGTGGCCCGCACTGCGTCCAAGAGCCTTTGCGCAACGCGCTCCTCACCTGGGCCCAGCACCCTGCGCATCCACCTAGGCGTGCAAGC
CCTACCACTGGGTCCTACTGTGATAAGAGCCTTTCGACAGCTCTCCCACCTCCAACAGCACACCAAAAANCACACAAGCGACAAACCCTCAAGGGCCCACATC
TGGGGTGGAATGGGTTCACTCAATCTCCACCCCCAACCACCACGCCAGGCAAAAGGGCAAGCTCAAGGGCCCACTGCTCCGGGCCCCCGGCCCGCCCCTGCGA
CCCCCCTCGCCATGCCTCACCGCCAGCCGCTGTGGGGAGGGGGGGGCGCCCCCGGACTACTATGAGAATACAACACAGGGGGGGCGGGGGCACTGCCCCAGGA
GAGCCCCATCGGGGATTTTTTGGG
>‘990729A-006.scf” came from CONTIG 6 at offset 0;
“E:\SEQUENCE\export\EST_db\990729a\990729A-006.scf’ (57>631)
GCACGAGGGTTTCCTGGCATCCTCAGGTGTACTGGNGATCCTGTGTCTATATTGCTCCCTCACTGAAACTTCCAATAGCTGCCCTGTAGGCTGCAAACTCAGA
TGCCCGTGTGGGCAAGTGATGGAGATCAGTGAATATCTGGGTACTAAAAAAAAAAAGCACCATAAAAACTAGACAGGGTTTTTTAAAGAAACTAGACAGGGGT
TTTTAAAAACTGGAAAGAGCATGTTCCCTGGCCTAGCCTCCATTTCCATGAAGGAGTAAAGGCCATCCTGACCTCAGGTAGTGAACAATCTATGTGCCTGACA
CACAGGTGCTGGGGCGAGGCTGNGAGTGAGAGAAAGGCCCCTCCTCTCCTGGGTGGATGCCAGATCTACAGACCTGAGCCCACAGGAGCCACACCAGGGCAAG
NAATACTGAAACATCTGCAAGACCAGATGTCAGNNTGTTTTAAGAAAGCCTGAATCAGATTTTCTGTGAATTCATTTTTGCAATGGGATTACAGCAGAAATCC
ACTTTTTATGATTATGTTAGTGGAATCTAAAAAAAAAAAAACTGAGGGGCCCGACCTATN
>‘990729A-007.scf” came from CONTIG 7 at offset 0;
“E:\SEQUENCE\export\EST_db\990729a\990729A-007.scf’ (57>619)
GCACGAGGCTAAAGCCATGCCTCCTCCTGCAGAACCAGATTCTCTCTCTTTGACCTTCCCCCTCCTGTTTCCCCAATGTTTAAAATGTTTGGATGGTTTGTTG
TTCTGCCTGGGGTCAAGGTGCTAATATAGACTTAAACGAATACATTAACGGAGCTAAACACAGACATAACCCAAGTCACGACATTCTTAGCTGTAACTCAGCT
CTCACGGCCTCTTGCTCACCCATGAATGGTCCCGTTTTCCTCTTGCCGCGTGCACCTCCACCCATTGTCTTGGGGGCACATGGGTGGAACACTTGATCTGCTC
GCGTCTGCCTTCAACACACATTGCATCTTCAGATTTTCTACTTTTCTGTGTCAAAACTAAATTCACCAAGTCAGACTTGTGTTAATTTATATCAGGGGATGGG
CTGNCAGGGGGCATGCCTAGNGGCCCCGAGAGGACAAGGGGAGAACAGCACGGATGTGGCAGGTGCTCTAGGCTGAGATCAGGGGGAGAGACGCGATCACAGC
AGACGCGATACGATCTTGTCGGGCGGACGCACAGAACGAGCGN
>‘990729A-009.scf” came from CONTIG 8 at offset 0;
“E:\SEQUENCE\export\EST_db\990729a\990729A-009.scf’ (50>328)
TGAAAACTTCGCTGTTCTATGCTTGGATCTTTGGGAGGGTCAAGTCTGGGCACCATACTTTGACAATCTTCCTCATCACCAACGATGGAAGCGGATGCTGGGG
GAGTTTGGCAACGGGACGTGGGNGTGTTACAGGCAGCAGAAAAGCAAATGAAGGACAAGCAGGGAGAAGAGCAAAGGCTACATGGAGGGAGGAGGAGGAGAAA
AAAGGCGAGGGAGAGGAAGAGGCAGACAAAGATGATACGAAGACAAGGATGAGGATGAGAGATAGAGAGAA
>‘990729A-010.scf” came from CONTIG 9 at offset 0;
“E:\SEQUENCE\export\EST_db\990729a\990729A-010.scf’ (56>610)
GCACGAGGGTTTATTGTTGCTTCTCCCTTAGCGTGTCCTGCTCAGGGTCAGGGCCTAGCTACAGATTGGGCTTCCAGAATTTAATAACTTTTTTAAAGTCAGA
CCTTGGCAAGAACTACAATATTTGGGGTTACATGCCCTTGACTTTTTAATAAACATCTCTTTCTAAGGAGATGCAGAAGGAAAGGAGGGTCTAAGCGAGACTC
TGGCCTACCTATAATAATGTCTGGGAAACAGGCTAAACTTAAGGCCATGGTCCACTCTTACTTCAGAGAGGGAGCTCAGATTGTGACATTACTGTTTTCCTTT
TCTGGCCTTTTCTCCTGGATAGAAGGAAGAATTGGAAATAGTTTGAGTAGTGGTTGGTTCATATTACCTCCTCTTGGTTGTGTTATTTTTTTTTTGCCCCCCC
ATATTAGGCAGGGCCCCCTGATGAGATGAGGGGGCTAGGCTACAGNGCCTGAGGGAAGACTATTTGGTTGAGGAGAGATCTCCTACAAAGNGAGGGGTAGAAC
CTCCTTCTACTATCTCTTACACAGATCGTCTATCCCA
>‘990729A-011.scf” came from CONTIG 10 at offset 0;
“E:\SEQUENCE\export\EST_db\990729a\990729A-011.scf’ (46>647)
TAGGAATTTTTTTTTTTTTTTTTTATCAAACAAAATCACATTTATTTCAATAGCTGCAAGTGCAAATACAGCAAGCCAGTACACAGTTCAGGACACTGCTTTA
AGTACAATGAAAACTTGATATACAAAAAATGAAAGAGGAAATAGCAGAAATAAAGCTCGATTTTTTTAAAAAGTTGAAGTAACCTGAAAACCTTTAGACTAAA
AGAGTCTTACATCAATTCATAAATGCCTTAATTTATAGATAACAGTGGTACATTGTAATCAAAAAGGACTTCCCAAAGAGTCATTTCGAACTGATCAGCTCCA
GATAAGAAATTCCTGGCAGTGGCTACTCTGGGATTCGGCACTCACTCATACTGTACAGACTGGTATAGTTCAATCCATGGCATTTTGATTTTTATTCAAATTC
CAANAAGGTAAATACCTTTTTGGTAAGNTCAATAGGAGGCCCACACCACTACCAAAGAGTCCTGAAAATGCCACTATATATTAAAGGTCTAAGACCATTTTGG
CTTAGGCCCATGTACAATGTGGGGAAAGAGTCATNANACTCGTACTTAAATCTTTGTAGATGCAAAGGCCCTAAGCTNCCAGAAACGNT
>‘990729A-013.scf” came from CONTIG 11 at offset 0;
“E:\SEQUENCE\export\EST_db\990729a\990729A-013.scf’ (55>549)
GCACGAGGTGAAAGGTTAATGGTTTGGCACATAGTTATTTGCATTTTAAAATCTCTGGTGGGGTGGTGGATGTTTAGGTGTGGGATTTAGGGGACAAGGAACC
GGTTTCTTAGCTAAAGTGTTCAAGTACTGTTTTGAACAGCTTATTCATTTTTGCCAGCCCTTCTGGACATCCCACACCCACCCATCTTTGCTAGGCTTAAAAG
ACTGAGCTATCCCCAGTGGCTGGCTGGCCTTGGGATTGGCTGCAACCTTGTTAGAACCACACAGGTTCCATCCTCACCTAGCCGCCTGTTACTTGAGGCGAGG
GGACAAAGACCGCCGCAGGACAGAGTTTAAGACTCAGCTTCAGGACCCTTGGHCCCCTTACCTAAACCCTAATCTACCCCTTCTTTTTCCTGGAGACTTTCAC
ACTCTTTTTTAGAGCGACACCAATATAAGCTCTATGCTAAAGGAGAGAAAGAGGCTGGGCGACTTTATTCCCACCAACAGTA
>‘990729A-014.scf” came from CONTIG 12 at offset 0;
“E:\SEQUENCE\export\EST_db\990729a\990729A-014.scf’ (49>564)
TGCTGCGGCAGCCACAAGAGGGGTCGGGCCTGGGAGCATCCCTGAATGAGAGCAGCCTGCAGGACATCATTCTGGAAACAGTGCCCGGGGGAGCCAGGACCCC
AGGAGGAGGCTGAAGAGGAAGGTGGGGGCGGTGAGGGCATAGCCCTCCCGGCCTCTCAGGGCACGTCGAGCCCTATCATCCACGTCGTGAACCAGACCAACGC
CCAGGGCGAGCGGGAGGTTCGTCTATTACGTGCTGTTCGAAGCCCGGGGAGAGCCCCCACCCGCCTCTGAGCCCCCCTCGGGGGCGTCATGGGAGAGCTTCAG
GAGCAGCGGAAGACCAGAAGTCCAGATGGTGTGAAGCTGCAGGCCCCACCCTTGTACCNCAGCCTGGAGCTGAGCCGCTGTGGCAGCGCCTAGGGCAGCCTGT
CACGATGCTTAGAGGAGCTGGAGACGGTGCACCTGAGCATGCGCACTACAGCGAGACGTCTTTTGCATCACTCTTGTGGCGAAGTTGTTTGAGGGGACCGNGG
G
>‘990729A-015.scf” came from CONTIG 13 at offset 0;
“E:\SEQUENCE\export\EST_db\990729a\990729A-015.scf’ (47>430)
TCGAGTTTTTTTTTTTTTTTTTTTGTAAAAAACATTTATTAATTAGCCACAACCTAACAAACCCTGCTCACCTGCTTCATCCCCTTTCCTGCTTGGGAGGGAG
GGCTCCTTGGTATGCAGAGCCACAAAGTGGGCGATCCAAGGGGAGATGCTCCTGGTGACTTCCCCACGGACCTTGCTACTGGCCTTGGCTCCTCACTGGTAGT
CGGCACCTTGGACTCAAACCTCATCTGCCTTPGGTTGCCTACTGGCAACTCTGGTTAGAGGGCTTGGCCCAGCTGCTGGCCCTTTCACCCCAGAAGTCTGTGC
AGCCTTGTGCCCAGCCAGGCCTACTTGAGCACAAGCATGGCCTCCGTGCCGTCCTTGGCGNGCAGGTAG
>‘990729A-020.scf” came from CONTIG 14 at offset 0;
“E:\SEQUENCE\export\EST_db\990729a\990729A-020.scf’ (362>694)
TTGTTGTTTTGAGTTCGTGGTCTCTCTTTGTCTTCTTTTTTTTCTTTTTATTTTGCTTTTTTTTTTTTTTTTTTTTTTTTTTTTTCTTTTTCTTTTTTTTCTT
TTTTTTTTTCTTTTCTTCTTCTTTTTTTTTTTCTTTTTTTTTTTTCTCTCATTTTCTTTTTCTTTTCTTTTTTCTTTTCCTTTCTTTATTTTATTTTTTTTAT
TTTTCACTTTTTTCTAATTCCACTTCTCTTCTTTTCTCCTCTACTTTCACTTGTTATAATGCTTCTCCATAGACTCATTAATGTCTAACTAGGATGAATATAA
TTCTCACCCATCTTTTCCTCCGA
>‘990729A-031.scf” came from CONTIG 14 at offset 0;
“E:\SEQUENCE\export\EST_db\990729a\990729A-031.scf’ (45>126)
TTGGGTTTTTTTTTTTTTTTTTTTTTTTTTTTTTTTTGTTTTTTTTTTTTTTTTTTTTTTTTTTTTTTTTTTTTTTTTATGG
>‘990729A-016.scf” came from CONTIG 14 at offset 0;
“E:\SEQUENCE\export\EST_db\990729a\990729A-016.scf’ (443>608)
TTCTTCTGTCTTTCTTCTTTTTTTTTTTTTTTTTTTTCTTTTTTTTTTTTTTTTTTCTCTTCTTTCTTTTCTTTTTTTTCTATTTCTTTTTCTTTTATTTTTT
CTTTTCTTTTCTATTTTTTTTTTTCTTCTGCTTCTTTTCTTTTCTTGTTGTTTTTTTTTGT
>‘990729A-017.scf” came from CONTIG 15 at offset 0;
“E:\SEQUENCE\export\EST_db\990729a\990729A-017.scf’ (46>592)
TGGCTGGCTFCATGATTCCTTAAAAAGGCCCCCACCACAGAGGGAGGACAAGAAGGCGGCTGAGAAGAAACGGGGAGGACAAAGACCAAGAAGAAGCACGGAC
AGGGAAGTCCAGGCGCCCGGGACGAGGAGGAGGGAAGACAATGAGGGTGGCGAGTGGGAGAGAGTCCGAGGTGGCGGGCCCCTCGYTTAAGGAAAAGCCAAAA
ATGTGTTGCCAAGGGAACTGAGATCACCCATGCTGTTGTCATCAAGAAACTGAATGAGATCCTACGGCACGAGGAAGAAAGGAACTGATCGTGCAGCCAGATG
AGCTGTGGGCTGCGGGTCAGTTGCCTTGAAAACACCTAGAGAGGGCGCATCGCAAGATAAGTTCACATCATTGCTTTTTTTACTACACCCCACCTGCTCGACA
TGAGACGGAGTGCAGAAGAGTTGACTGCTCATGAGTGTGGCATCTGTTGCAACCTACTCTTGTGAGAGAATATGGAAAGGAGACGCTACCAACACCCGGGTCG
GCTGCTTTACTGGGACGAGTAGATTCCAAAGCG
>‘990729A-018.scf” came from CONTIG 16 at offset 0;
“E:\SEQUENCE\export\EST_db\990729a\990729A-018.scf’ (55>562)
GCACGAGGGTTTGTGGAAAATAACTCCTTCACTTCAGCTTGTTGTCATGGAAAGGGTGTTGACTTCCGTGTCTGGAGACTTGATGAGTCTTGGTGCTATCAAT
TACAGTGTGAGTTTGGGCAAGGCACAGGTTGCCCTAGATTTATTGCTTCATTTCTAAAGAGAGGAAGTAATGCCTGACCTGCCTACCTCACAGGGCTATTGAG
GGGATCCAGNGAGACAGGATACACGTAAATGTGATTTTGTAAGATGAAAAGTATTGGACGGGGGAAAGAATTTAAGCCATAGATTTTAGACTATTTTTCAAAT
GACTGAAAGAAATTTTGATTTCCTGGCTTACCAACCTCATAGAGAGAGACAATGAATTCAGCTGCTAAGGAAAAAACTTAGNAGTCTATGTTCTTGTCAAGGG
CCCGTGGTCTGCGCGCTACATTTCTCCTCAGCAGCGACTGCCACACACTGCGAGGAGGGAAAAGAAGAGGNGACAAGATGAGA
>‘990729A-019.scf” came from CONTIG 17 at offset 0;
“E:\SEQUENCE\export\EST_db\990729a\990729A-019.scf’ (49>570)
GTGAGGATCTCGTCTCTGCGCCTIGAGCCATGCCGTCCAAGGCCCTCTGCAGTCGGTGCAAGTCTTCGGACGTAAGAAGACGGCCACAGCCGTGGCGCACTGC
AAACGAGGTAACGGCCTCATCAAGGTGAACGGACGACCCCTGGAGATGATCGAACCGCGCACGCTGCAATACAAGCTACTGGAACCTGTTGTGCTCCTGGGCA
AGGAGCGATTTGCTGGTGTGGACATCCGCGTCCGAGGAAGGTGGTGGTCACGTCGCCCAGATTTACCCATCCGCCAGTCCATCTCCAAGCCTTGGTGCCTATT
ACCAGAATAGGGGATGAGGCTTGCAGAAGAGATCAAAGACTCCTATCCAGATGACCGACCTGCTGTAGCCGATCCCGCGCTGCGATCAAAGTTGGAGGCCGNG
CCCGGCCGCTCCAAAATCTCCGTAGCCGGCTGAAGCACGTNCCTTCACACTTTATAAGTTTGGATTAGTTTAGAAAAAAAAAAAAAAAAAATGGGGGGCCGAC
CCATGCT
>‘990729A-021.scf” came from CONTIG 18 at offset 0;
“E:\SEQUENCE\export\EST_db\990729a\990729A-021.scf’ (46>562)
TGGTTTTTTTTTTTAGTTTGGTTGGTTTTGGTTTTTATTAACAGCTTTATTGAGATACAATTCACATACCGCAGACTTTACTCATTTAAAGAGTTAACAGTTC
AGAGATTTTATATATTCACAGAATTGACCATCACCACAGATTTTTAACATTTTCAACACCCCCAAAAGAAAGGCGCTACCTTGTAGCAGTTACTCCTCAGATT
TTCCCCAACCCCCCAACTACAGGCAACCACTAAACCACATTCTGTGTCTGACTGGCCAACTGGGGACATGACTATAATGGGATATTATGTGGCCGTGTGTGCG
AACCAGCTCAGGGCTTAAGGAGCAGGAAGCAAAGGAAAGGCTGGATGTTGCCGCAGAGACGAAGGCGAGGCAGGAGCTCGGGTGGGGAGGCCAGGCAGAGGAG
CAGGAGCGCGTCTCTACCAATCTNGCTCAGAGACAGTAGGCTGGGCGCGGCCCGGGCTTTCACCTCTCGCTATCAAGGCGGACACATCCACAAAAAACCGCG
>‘990729A-022.scf” came from CONTIG 19 at offset 0;
“E:\SEQUENCE\export\EST_db\990729a\990729A-022.scf’ (197>625)
ACTAGGGGAGAAAACGGGCACAACGCTGTAATAAGGCAAGGGATGACTTTTATCCCCCCCCAAAAGGGGGGAAGGGGGGAGTCCGCCCCCCCGGGGAACGGGG
AACAGAGGGAGGCAGCGGGGGGTCTCTGGTGGTGCTTTCGTGCCAGCACCCCCTATCCGGGCGCGGGGGTAAGGGGAGTCCCCCCAGGAGACCCCAAAAAAAG
AATTTTTTCTGAACCTTTAATTGGGCCTGTCTTTAATAATTGTGGGGGGGGGGGTTCTTTGCTCCACCACAAGAGACGGAGGGGGGATATGCTGGGGGGGGCC
GGCAGAGGAGGTTGGCCCCCGGGGCAGACCTGCAAGGCGGGCGCGGCGCCGAGGAACAAAGCGGCCCGCCCCCCCTCCTGGCCGGGCCTCCCCCCGCCTGGGG
GGGGGGGGGCCACGT
>‘990729A-023.scf” came from CONTIG 20 at offset 0;
“E:\SEQUENCE\export\EST_db\990729a\990729A-023.scf’ (48>557)
CGCGAGCCCCAGGACCTGTGTGCAGTAGCCGCGCATCCCGAGCCGGACCACGTCGGAGTTCCTCTGGACCCAAGACATCAGAAGCCATGTCGAAGCACCACAG
CGACGCCGGGACGGCCTTCATTCAGACTCAGCAGCTGCACGCAGCCATGGCCGACACATTCCTGGAGCACATGTGCCGCCTGGACATCGACTCACCGCCCATT
ACGGCCCGAAACACCGGCATCATCTGTACCATCGGCCCAGCTTCAGAGCAGTGGAGACATTGAAGGAGATGATTAAGTCTGGATGTATGTGCTCGTTGAACTT
TCTCATGAACCACGGTACACGCAAGACCATAAGATGACGTGAGGCCGAGGCTTGCTCAACCCTTCTTTCGGCGGGCGGGCCTGACACTAGACGAGATCGACTG
GTCACAGGCGCGCCGTGGGGGGCGAGAGGAGCCACGAACACTGCAGCTACTGAAGGGCGACTCGGCGACCAACATGAGGGGGGGCGAGATACGAGAGC
>‘990729A-024.scf” came from CONTIG 21 at offset 0;
“E:\SEQUENCE\export\EST_db\990729a\990729A-024.scf’ (101>231)
CGCGGGTTGGGTGTGCTGGTGGCTTGGTTTTTTCTGCTGATTGTCTTGCTGGTTGCAGTTGTCTTTGTCGTTGTGTTTGGTTTTTTGTATGTTGGTGTTGTTT
GTGTGGCTATTATGGTGTGTGTGGGTGTA
>‘990729A-025.scf” came from CONTIG 22 at offset 0;
“E:\SEQUENCE\export\EST_db\990729a\990729A-025.scf’ (31>550)
GTCCCCCGGGCTGTGGTAGAACCTTTATTTTGTGGTGAATAATCCTATAATTGCTTGGAGTTTATTTTGTTTATTATTTTCTATTAAAAAATTGTCAGACCTT
AAAAAAGAAAAGTAAGGTTTAAAGCATCATGTTGGCAAGTCATTTTTATGTGTGGGGGAATTTGGCAAATAAATTTTAGGGGGATTGTTCTTTTTTCCTTCTT
GGACTTTTTATCAGGGAGAACATGTCAGGATAAAATTTAAAACTAAATTAATCATTGGGGGCGGTGAAACAATGAATAATATAGGGTGGGGGGCTCTATTTTC
TTATTCTGTTGTATGACAGGACTGGATCGCTTTGTGTTTTTAAATATTAAAAGATAGGGGGTTGCCACTTCTGGTGGTTGCTTTGGACTGTGGCCCGGGCAGC
CGATTGGGGGAGGAGCAAAAAATGATACTATTTGTGTGGGGCAGGGCTGGTGGGACAGGAACATTTTTGTGTGTGGGTGTTTATTTTTTAGGGAGAG
>‘990729A-026.scf” came from CONTIG 23 at offset 0;
“E:\SEQUENCE\export\EST_db\990729a\990729A-026.scf’ (44>536)
TTTGTGAGTCTCATTTTAAGTGGCCTTGATATTTAAAACTATTCCTGCCACCAATTCTTTTCCTTGGCCACTTTTTCCTCGTGTCTCTTCCTGCATGCTGCTT
TATTTGCTTCCCCACCACCCTGGGGTATGAGTTATTTAAAAATGAAAGGGGTAAACTAGTGGGGTTGTGGAGATTAACATAAAGCACTGATTTAACTTGCTAA
GTAAACTGAAAGATAAATCCTGACTGCCTACTATCCAATGTCAGTTAACCGCGTCCTCCCCTTCATTTTCTCAGTCCCCTAAAGCTTCTGTCCCGGATTCCTT
CAGCTCTTGACTTCACGTTGCTCTTCTCTTCTCCCGCTTTGCTCCTTCCTGTCTNCATGAGTTGATGAAATGGAAGATTGCATGCACTAGGTTGGAGGGGGTG
NGGTNTGTCTTTCTACTAAGGTATAGCCATCACTTCCTAGATAAATACTACCTAAATTGATGTCTCATTTG
>‘990729A-030.scf” came from CONTIG 23 at offset 0;
“E:\SEQUENCE\export\EST_db\990729a\990729A-030.scf’ (45>376)
TTGGTGAGTGTCATTTTAAGTGGCCTTGATATTTAAAACTATTCCTGCCACCAATTCTTTTCCTTGGCCACTTTTTCCTCCTGTGTCTTGCTGCATGCTGGTT
TATTTGCTTCCTCCCCACCACCCTGTGGAATGAGTTATTGAAAAAGGAAAGGGGTAAACTAGCGGGGTTGCGGAGATGAACATAAAGGACTGATGTAACTTGC
TAAGAAAACTGAAAGATAAAACCTGACTGCCTACTATGCAACGGCAGTTCCGCGTCCTCCCCTTCATTTGCTCAGGCCCCTAAAGCGCTGCCCCGGATTCCTT
CTTTGCTCTTGACTTCACTTG
>‘990729A-027.scf” came from CONTIG 24 at offset 0;
“E:\SEQUENCE\export\EST_db\990729a\990729A-027.scf’ (54>547)
GCACGAGGATCTTGCTGCTTATATGTACCTGTGCTTATATCCGATCCTTGGCACCCAGCCTCCTGGACAGAAATAAAACTGGGTTTGGGTATATTTTGGAAGT
GTGCCAGAATTGGTGAACGGAAGAGTCCGTATGTTGCAGTGTGCTGTATCGTGATGGCCTTCAGCATCCTTTTCATACAGTAGCTTGGAACAACGCCAGAATT
CCAGGCGCTATCAGATTTAAATATGACAAAAAAGGACGATCTGCCGAAAATAGAGGAAAGAATGGTTAACCCTTATCTCTCAAATTGAAGAGCTACACTCTCA
CTGCGTTCTCCTTTTTGTATTGGACCAAGTCTTATAAAAATTAGAGTAACATTAATACCGAGTGAAATGGNCTGAACATCACCCACACTNCGCTCATATACAT
TTGCTTGTCATCTTTGGCTGATCAGCTTAGGAGATCTTAGCCAAGAAAAACAAAGTAATATAGTCCCTTCTGGATGAAG
>‘990729A-029.scf” came from CONTIG 25 at offset 0;
“E:\SEQUENCE\export\EST_db\990729a\990729A-029.scf’ (55>468)
GCACGAGGCTGGGCTGTGCAAAGCTGGGTTTGCAGGAGATGACGCCCCCCGCGCCGTCTTTTCATTGTGGGGCGGCCTCGTGACCAAGGGGGTGATGGGGGGA
ATGGGGCAAAAAGACAGGTATGGGGGAGATGAAAATCAAAAGAAGGGGGGGGATCTTACTCTCAAATACCCCATTGAACACCGCATAATTACTAACTGGGGTG
ACAGGGAGAAAAACTGGCACCACTCCTTCTACAATGAGCTGCGGGGGGCCCCGAGAACACCCCACCTGTCACAAAGCCCCCCTGAACCCCAGGCAACAAGAGA
AAGACCAAAACAAGTTGAAACTCAACACCCGCAGGACGCGGCCTCAACTGGCTTCCTCTTTGCTTTGGCGACAACGCAGGGCCGGATTAGGAGGGGACCACA
>‘990729A-032.scf” came from CONTIG 26 at offset 0;
“E:\SEQUENCE\export\EST_db\990729a\990729A-032.scf’ (58>61)
TTGC
>‘990729A-033.scf” came from CONTIG 27 at offset 0;
“E:\SEQUENCE\export\EST_db\990729a\990729A-033.scf’ (54>518)
GCACGAGGGTGGAGTCCCACTGCTGTGCCTCGGATGTGAAGCTCAAGCTGTACGACCGGAGTCTGGAGTCAAACCCGGAGCAGCTGCAGGCCATGAAGCACAT
CGTTATGGGCACCACCCGCCCCGCCCCCTACATCATCTTTGGGCCTCCGGGGACAGGCAAGACTGTCACCCTAGTGGAAGCCATCAAGCAGGTGGTGAAGCAC
TTGCCCAAAGCCCACATCCTGGCCTGCGCTCCGTCCAACTCAGGGGCTGACCTCCTCTGTCAGGCCTCCGGGTCACTTACCCACTCCATCTACGNCCTCTGGC
GCCCACAGGATATCCGCTGGCCCTGAGACTCAGCCCTGTGTTACTGGAGCAAAGAGGGATTTGTTTTTCTTCAGAAGAGCTCAGNATTCGNGCTTATTACACC
TCTCCTGCAGCGTGGCTCAGCACTTCCTCATCCTCCACCTCTTTCGCAGCGGC
>‘990729A-034.scf” came from CONTIG 28 at offset 0;
“E:\SEQUENCE\export\EST_db\990729a\990729A-034.scf’ (46>481)
TGGAATTCGGCACGAGGGCGAGAGGAGGGGGCTGGGCCGTGGGGAGCCCCGCGGAATGGGGCACCGTGGGCTACTTCTGCTGATGCTGTTTAGGCGGGTTCCT
CTGGGACGCATTCACCGGCTGACGCTGACGGGGGAGAAGCGAGCAGATATCCAACTGAACAGCTTTGGTTTCTACACCAACGGCTCCGTGGAGGTGAATCTGA
GCGCTCCTGAGGCTAGGCCGCCAGGATACAGAAGAGAAGGCCCCGCTGTGGGGGTCAGGCTGACCCGGTGAGATCTGCAGCATTCGCTCCTATCAATCGGGAC
TCATGAGTGGGTCTACGGAAAACAGAGCAGCCCTGGTCTTACTCACAAACAAGGATTGGAGCCAGTCGAAAGATGGGAGCAAAAAATATTCTCTTGCTGGCTC
CTCGCATCACCTCAACAGGCTCCGA
>‘990729A-035.scf” came from CONTIG 29 at offset 0;
“E:\SEQUENCE\export\EST_db\990729a\990729A-035.scf’ (49>380)
GGGGTGGGAGCAGGAGGCACGCGGGGTGTGAGGCCACGCATGAGCGGACGCTAACCCCCACCCCAGCCGCAAAGAGTCTACATGTTTAGGGTCTAGACATGTT
CAGCTTTGTGGACCTCCGGCTCCTGCTCCTCTTAGCGGCCACCGCCCTCCTGGCCCTGCTGGCAAAGAAGGCAGCAAAGGCCCCCGCGGTGAGACTGGCCCCG
CTGGGCGTCCCGNGGAAGTCGGCCCCCCTGGTCCCCCTGGCCCCGCGGGGAGAAAAGGGAGCCCCTGGGCTGACGGACCTGCTGAGCTCCTGCACTCCTGACC
TCAGGTATTGCGGACACGAGGGGG
>‘990729A-036.scf” came from CONTIG 30 at offset 0;
“E:\SEQUENCE\export\EST_db\990729a\990729A-036.scf’ (55>461)
GCACGAGGGACTGACAATGATCTTATCAATATTCTTGGACCCTTTTTATCATCTTTCAACTAAAAGTTTCAAAACACAACTTTTATCACAATCCAGAACTGAC
ACCAACAAAAATATTACAAAACACCCCTTGAGAAACAAAATGAACGAAAATTTATTTACCTCTTTTTATTACCCCTGTGATTGTAGGTCTGCCTCTCGTGACC
CTCATCGGACTATTCGCAAGCCTACTATTTCCACATCAAACCGACTAGAAGGCATCGCTTTGTACCCTCCACATGAATACTCTACTTGTATAAAACAATATGA
GTATCACAATCTTAGGACAAACAGACATTATATTATATCTTGTCCTTTTGGGAGCACAAACTACTAGCCTTACCCCCTCTTCCACCACACACACAT
>‘990729A-037.scf” came from CONTIG 31 at offset 0;
“E:\SEQUENCE\export\EST_db\990729a\990729A-037.scf’ (55>532)
GCACGAGGGCCCTCGGCCCATTTCGAGTTCAGACAGCGACAGTGATAAATCCACAGAAGACCCCCCAATGGGTGAGCCTCACCATCACCCAACCCCTCCAATA
GAGTGAGCGAAGCTGCCATCCTTCCCAGCATAACTCCCACCTAGCCCTTCATTTGCCCATAATATATGAGAGCTAGAAGGGCCCTTAGGAAGCCTGTCATTCA
ATCCCCTCACTTTATAGATGGGGAAACTGAAGCCCAGAGCCACTAACCCAACCAGATTCCCATCCGGGGGCCCTTCATTTATCACTCACCTTTTCTCTTCTCA
TTCTCGTGGGGAATATCCTTTGCCACTGTGTCCTAAGGCTAGTAACTGCCAAGGGAAGTGCAAGGGGGGGGCAGGGCTGTGGGCGCTGGGGTGCACACGCGCC
AGAGTGGCTTGCTGGTGAGGGTGAGGAATTTCAGCCAGACGGAAAGCCAGCTTGAGACCCCT
>‘990729A-038.scf” came from CONTIG 32 at offset 0;
“E:\SEQUENCE\export\EST_db\990729a\990729A-038.scf’ (49>243)
GTATGAAGTAAGGTTTTAGTTGGTTCAAATGATTCCAAATGGGGGGCGGTTGGCTTGGCATACAGAGGACACTCTGGGGTGTGGGAAGGTGGGGAAGGAAGGG
AAGGAGAGGGCCGCCTGCCGGGGTTGTGTGTTTGAAGCTGATCTCCCGAGCGGCCCCGTGGCGCCTGGCCTGCGTTTTGTGTGAGTTGAAAG
>‘990729A-039.scf” came from CONTIG 33 at offset 0;
“E:\SEQUENCE\export\EST_db\990729a\990729A-039.scf’ (1>603)
CCGGGCGGCGGGCGCTCTATAACTATTGGGTCCCCCGGGCTGTTTTTTTTCGGCACGAAGATGATGTTGAATGGTGTTGTGTGAGACCTACAAGACCTTTTAT
TAACTAACACCCAAAAAAGTCGTGCTTTTATTATGGGGGACGGAATACAAAAGGAGGAAGGAAAGAAACACCTGGGGGAATGGGCGGATTGGGCCTTAGAGGA
GGGGGTGAAGCAGGGGUGGGGGTTTGTGAGTTTTGTCAAAGGAACGCACTGGTCGTAGCCAACACCCCCCCTTCCAACAACACAGGAGATGACTGTACACATG
GACATCACCTGATGGCCAACACTGAAATCAGATTGTTTTTTTTCTTTGCAGGCAAGGAGGGGAAGCCCTTTCCAGCCGGAAAAACAAGACCGGGTGCGAGATG
TGGCGAACACATGTACTCCCTTTGGCAATTCACACTCAAATGAAGATGTGGGGAAACCACCACCCCTGCGGGGTGCCCTCCTTAATCCTGCTCTCATTGGAGG
TAATTGGCTTTAGGCTGGTCCGTTGTCGGGGCGGCGACCTTGTGGGGTCGTGCTTGCTGTCGGCCCGCCCCCCCCGCCAAC
>‘990729A-040.scf” came from CONTIG 34 at offset 0;
“E:\SEQUENCE\export\EST_db\990729a\990729A-040.scf’ (39>122)
CGGGCTGCAGGAAACCGGCATACGAGGGTTGAAGTTGTATTGGTGTCTTGTAGACAATACTAGAAGTGTTTTTATTATTTAT
>‘990729A-041.scf” came from CONTIG 35 at offset 0;
“E:\SEQUENCE\export\EST_db\990729a\990729A-041.scf’ (48>56)
TTTTTTGGC
>‘990729A-042.scf” came from CONTIG 36 at offset 0;
“E:\SEQUENCE\export\EST_db\990729a\990729A-042.scf’ (46>511)
TGGTTTTTTTCAGTTAAAAAGGCAAAAACTTTATTTAGTTTTTGGGGGAAATACAAGATGCATGTAAACATAAAATACAAAACAAAACAACCCAAATCTTACA
GTCTAGAAGCATGCCAAGACAGAACATTTTCTGCAGACCAGAGTCCCGTCAAAAGGATAAAGGGCACCTGGAAAGGGGGGGGGCAAGGGGCTGGGTCCCTTCC
CCAAGGACACTGCTTTTTGTGATGAGAACAACTGAAAAAAACCAACCCATTATAAAAATATAGAAACTGAGACAGTTTACACCACCTGGGCCTGGAATTTTTA
GCCTCGACTGCCTGATTCATGTTCTTTCTTCGTTCTGTGTGAGGAGAAAGGGATGACCCCGCAGCCCCAGGCCGTGGGCGGGGGGGGGGGGGCACGGGAGAGG
CCCAAACAACAGTGCTCGGCAGCAGAACCCAACCACCTACAACGCCCCACCGCT
>‘990729A-043.scf” came from CONTIG 37 at offset 0;
“E:\SEQUENCE\export\EST_db\990729a\990729A-043.scf’ (55>565)
GCACGAGGCTTGCCCGCCACCCGTGCGCCCAGTCCGAGAGGCCAGCCCAGTTCTCCCGGTCCTCACTGCCCGCCGGCCGGCCCGTCCCCCACTGCAACCATGG
ACGCCATCAAGAAGAAGATGCAGATGTTAAAACTGGACAAGGAGAATGCCATCGACCGCGCAGAGCAGGCTGAGGCCGACAAGAAGCAAGCTGAGGACCGCTG
CAAGCAGCTGGAAGAGGAGCAGCAGGCCCTCCAAAAAAGCTAAAAGGACGGAGACGAGNGGAAAAGATTCTGATCAGGAAGGATGCCAGAGAAACTGAGCAGC
TGAGAAGAAGCACTGTGCTGAGCAGAGGGCCTCCTGACCGGCATCACTGGAGAGAGAGCTGACGGCCAGAGCTCTGCTCAGCCGAGAGCGGGGAGTGAAAGAG
TGAGAAGGAGAAGATGAGTCATGAACGACTTGAGAGAGAAAAGAGTGAGAAGCATGAGAGCACCATGCGGATCGACGAATGGAGGGCGAACGGATCGAG
>‘990729A-044.scf” came from CONTIG 38 at offset 0;
“E:\SEQUENCE\export\EST_db\990729a\990729A-044.scf’ (49>550)
CCAAGGACGCCTTATGACTCCGTTTACTAAATGTACCACTCAGACCTGCAGGCCTTGCTTGGGGTGGGACCTTGATACTGGAGTTTTGGTTATTAATCCTTCC
TCCTCTGACCCTAAATTCAGAACACAGAAAGGGATCCAGTCAGGGAATGGAAGGAAATCTCACCACGAAAGGCTTAAGTAAACTCTTAAAAAGCAGTTGTATT
GAGGTATTTTTTTAAGTGCACAATTTGATAAGTTTTGACATCTATATGAAATCATGGCCACAATCAAGACATAGGTGTATCTCTCACTGCCGNACGTTACTGC
TGAAGACGTCGTTTTGCTATTACTCTCTCAGAATCTGAGCACTGNAGATCAGAGACTGATTACAGGGCCCTATGACCTGTCCTATCTTCTAAGACCGCGAGAG
CCACAGACTACAGCTCTGAAGAGATAACGGCCAAAGACTTGTACGAAACAACAGCTTCTCAGACTTTATATGGTGATAGACGAGACGAGAA
>‘990729A-045.scf” came from CONTIG 39 at offset 0;
“E:\SEQUENCE\export\EST_db\990729a\990729A-045.scf’ (55>561)
GCACGAGGCTTATCTCCAGGTGCGTACGGGAGCTGAGGTGGGCGAAACTTCGAGGGGTGAGGAGAGGGTGCCGGGATCCAGGTGTGAGAGAGGGGTGGGCGTG
AAGGCGAAAAGAACGGGCCCGCCCCTTTCCGGCCTGGAAAGTAGTTTCTGTGGGTCCCTGGGAACGTCGGAATACCAGATCTCGATCCGTGGGGGCGGGGTCC
CTGGGGGAACTTGAGCGCCCCCTTCTGGGAACCGGCGGGTCTGTTCGCAGGGACTGCTGTTGGGGCCTGATTGGTTAGACAGACGTTCCCCGAAGCCACGGGA
AGCCCTACCCGCGGGGCGTGGGTGGGGGATCCCTACTPAGTACTCCTGCCTCTCCTGCATCGCAGCCCCTCCCTAGTGCAUGTCCCTGTCCGGGCCATNAGAC
ATGCACCACCACGCGGCGCTCTGTTGAGAAGGAAGGACCTCGTCTCAGCTTGCTGGGAGAACCGAGCCCTTGCTCGCCACAACGGAAAAGAGAG
>‘990729A-046.scf” came from CONTIG 40 at offset 0;
“E:\SEQUENCE\export\EST_db\990729a\990729A-046.scf’ (55>533)
GCACGAGGCTGACATCGGCCTCCTGCAGAGCCTCCAGAACTTGGCCGTCACGGCCAACCGGATCGAGGCGCTGCCACCCGAGCTCTTCCAGGGCCGGAAGCTG
GGGGCCCTGCACCTGGGCAACAACGTGCTGCAGCCGCTGCCCTCGCGGGTGGGCGAGCTGACCAGCCTGACCCAGATCGAGCTGCGTGGCAACCGACTGGAGT
GCCTGCCTGTGGAGCTGGGCGAGTGGCCGCTGCTCAGCGCAGTGGCCTGGTGGTGGAGGAGGACCTGTTAACACCCTGCCCCTGAGTGAAGAGCGCTCTGGAG
GTGACAGGAGCAGCCTGAGTCCATGCATGAGCACGGTGCCCTGGGGGCGCCGCACCGACCCCAGCAGCCTGACCCGAACCAGAGCGACGACACCAGCACCTGC
AGAGGCGCGGGCTGNCGACAAGACGACTGAGGTGCCCTTTTCTGGATAGCCCCAGCGGCGCGAGGAA
>‘990729A-047.scf” came from CONTIG 41 at offset 0;
“E:\SEQUENCE\export\EST_db\990729a\990729A-047.scf’ (55>544)
GCACGAGGCTCGCTCCGGTGTCCCCGCGCCAGAGACACAGCAGCGCTCCCTCTGCCCACACCCACCGCGCCCTCGCGCTCGCCTCTCCTTCCGGAGCCAGTCC
GTGCTACCGCAGTCGCCCAGCCCACCACCACCCTCTGCAGCCATGTCCACCAGGTCCGTGTCCTCGTCCTCCTACCGCAGGATGTTCGGCGGCCCCGGCAGCG
CAGGCGGCCGAGCTCCACCCGGGCTACGTGACCACATCCACCCGCACCTACAGGCGGGCAGGCGCTGGCCCACCCCGCCGCACCTTACACCTGTCCCGGTGGC
GGGTCGCCAGCGCTCTGCCGGCGCTGGGGGGGGTGCGGCGGCGGTGTGAGACCGGGGACTGCTGTGGCAGCCTCACACGATCAGACACCGACCACAGAGGAGC
GAGACCAGACCGTCCACACCGCAGGCGCTCGACAAAACAACGTGCGACGAGGCTAGGCAGCAGGGCGGGACTTCAGAG
>‘990729A-048.scf” came from CONTIG 42 at offset 0;
“E:\SEQUENCE\export\EST_db\990729a\990729A-048.scf’ (46>593)
TGGAATTCGGCACGAGGCACCAACCGATTCGACCAGTTGACGACGAATCGGACCCCTTCGAGGTGTTGAAGGCAGCAGAGAACAAGAAAAAAGAAGCCGGCGG
GGGCGGCGTTGGGGGCCCTGGGGCTAAGAGCGCAGCTCAGGCCGCAGCTCAGACCAACTCCAATGCGGCGGGCAAACAGCTGGGTAAAGAGTCCCAGAAAGAC
CGCAAGAATCCGCTGCCCCCCAGCGTCGGCGTGGTTGACAAGAAGGAGGAGACGCAGCCGCCTGGGCGCTGAAGAAAGAGGAATAAGACGTGTTGGAAGAGAC
CTGATCAACAACTTCGGGTGAAGGGAAGATAATGAAGGAGACCGAAGGCGACCACCTGTGAAAGAGATTCGAAAGCCATTGAGAAAGGTNGAGGAGAGATTTC
CGTGATGACGATTTTGCCGCCTTCCGAGCCGGTGGTCTGGAGGGCGGGAGCCGGACGGGAGGGCGGAGAGCTTGTCTCGGCAACGGATTGTAGCTGGGAGGGG
ACGCCGAGCGGGCAAGGGGGGGATGAACC
>‘990729A-049.scf” came from CONTIG 43 at offset 0;
“E:\SEQUENCE\export\EST_db\990729a\990729A-049.scf’ (49>505)
TCTTGCTGCCGGATCTGGCTTCCGTGGGGACCCTCCCTTGATGAATATGGCCGTCCTTTGCTTATTATCAAGGATCAAGACCGCAAGTCTCGATTTATGGGAC
TTGAGGGCCTCAAGTCTCATATAATGGCAGCAAAGGCTGTGGGAAATACAATGAAACATCGCTTGGACCAAATGGGCTTGATAAGATGATGGTGGATAAAAGA
TGGAGACGCGACCGTGACCAATGACGGCGCCACCATCTTAAGCCTGAGGACGGTGACCCCAGACGGCAGCTGAGGCTGAACTGCCAAATCCAGATGTGAGAGG
GAGAGGACCACAGAGGGTTGGCTGCTGCGCCTGGGGAGAGCGAGAGAGCTGGCCGGGATGACCCACGAGGCCGCGATACAACGCGGCGCTGCTTGAAACCGAC
AATANGACGGGCTGAGACGAAAACGTGCCCCTCAAGCAGACCGCT
>‘990729A-050.scf” came from CONTIG 44 at offset 0;
“E:\SEQUENCE\export\EST_db\990729a\990729A-050.scf’ (46>198)
TTGGGTTTACATCTCCCCACATTTCATACCAGTATTCCAACAGATTCTTTATTACTTAAACCCAAAACCACTTCAAACCATTACCCTTGGATTGGGACTTAGC
CTTTAGCTGTGCACACGGAGAAAATTCGCGCCACATTTGGGCCTCCACAC
>‘990729A-051.scf” came from CONTIG 45 at offset 0;
“E:\SEQUENCE\export\EST_db\990729a\990729A-051.scf’ (45>193)
TTTGAATTCGGCACGAGGCTCGGGTTTTTTTTTTTTTTTTTAGGTTTTTAAATCAACTTTTCCAATAAGCAACTAGGGTTAGCCCACATAAATATGCTACCAA
TAAATGAGAACGCTTAATGGCTTATTACATGCTATGTATGTGCTT
>‘990729A-052.scf” came from CONTIG 46 at offset 0;
“E:\SEQUENCE\export\EST_db\990729a\990729A-052.scf’ (45>513)
TTGGTTTTTCCTTACTTGGAGATCTTCTATATAAATACTGTAAATGACATCTTTTCGTACGGACCTGTTCGAGCGTTGCTTTCCAGAGCCCACGAACAGTGCT
GGCCCGATCTTGGGCTCTGGCTGCCCCATCCGCGTGGAGCCCTTGCAGGAAGCCCCGGCGAGCAGAGGAGCCGCGCCTGGGTCCCAGCAGCGCTCACTAGTCT
GTCACTTGGCCCGGNGCGGGCTCGTCGTCATCTTCTTATGGCCAGGATGTACTGACTAAATCTGGTTAGCAAATCAGACCTCCTCCCTTCAGAGCATACAGGT
CATCTCCTCAGCTTCCTCCACCGAGCTCGTGGGAGGGGCGGACCCCGGGGCTTGAGGGGAGGGGGAGCTGCCTCCTGGGAGCTGAGCCCTCCAGACAAACCTT
CTTCTCCTCGCATGCAACAGGCAGACCCGCTTATCGTCATCACTTCAAAAGAAGAGC
>‘990729A-053.scf” came from CONTIG 47 at offset 0;
“E:\SEQUENCE\export\EST_db\990729a\990729A-053.scf’ (56>573)
GCACGAGGAGAGAACTAGTCTCGAGTTTTTTTTTTTTTTTTTTACGAACGAGGCAATTTATTAACCCAGCATCATTTGTTCTAATGCTTCTTGTTGGCAGCTG
CCACCTGTCCAGCGATTCTGTCCAGATCTCTCTGTCCCTGAGGCGTCAGTTTGCGGCCCCCATCTTGGTCCTCCACCATTTTCAGCCCCTCCAGGGCTTGGAG
GACCCGCCGGGCCACGCTCTTGGAGCCTCTGCTGAAGTGGGTGGGCATGACGCCGTTCCTCTGGCGCCCCCCATAGATCTTGGTCATGGAGCCAACCCCACGC
CACCCCGGGGTACAGGGGCCGNNGGCCGGGAGCGCTCGGGTGTAGACCAGTCTCANGTAGGGAGCAGTTTTTATGCTGGCCGCTGACGCGTCCACCATCAGGA
CTTCAGCTCCCGACTTTTGGGAAGCTGCAAGCTTGACACTCTGCTGTGAATCTTAGGCTCTGCCTCAGGCCGGGGTCCGTGTAACACGAACGGGGACGTCCGC
>‘990729A-054.scf” came from CONTIG 48 at offset 0;
“E:\SEQUENCE\export\EST_db\990729a\990729A-054.scf’ (56>577)
GCACGAGGGATCAAAAAGTAATAAGCAACCTTTTGAACCTATGATTATTTATGCACACTTCTAGTTTTGTTTTGATATTTAAGAACTGTTGATCATCTAAAGT
TTCTATGCACAGCACTGGCATCTTCAAGCAATTTAAAATTTGAGAATATCCATCAAAAATTCTTACCTTCTGGAGATGACTTTGTCACATATAGAGTTATTGA
ATAAGCATGTTGTGCACCTGGAACCAACATAGGGCTGTAGGTCAATTATACTTTGAAAAAAAAAAAGTTCTTGCCTTCCTTATTCTCAAGCATCCCAAATTTT
GCAACCTCCNTCTTTCTGGCCCCAATCACCAAAGAAGATGGACCCTGCCCAGCCCTTGCTTTGAGCCCCTCCCTCCTTCCCTCAGCTTCCTGAGACGCTATAT
GAATGACCACACNCACAGAAAAACACTGGTTTTCCYYAAGGTAGTTCCGGGGGGGGGGAGGCCCAGACCAGAGGCCGGACAANACAGAAATGGAGAAGACGNT
TC
>‘990729A-055.scf” came from CONTIG 49 at offset 0;
“E:\SEQUENCE\export\EST_db\990729a\990729A-055.scf’ (56>534)
GCACGAGGCAAGTCTCTACATTTGTTCTCAGGCAGGTTCTAGTATTTTTAGGGGCAGGGTCAATCGAAATTGACCCTTCAGATTAGTCAATCCAAGTTACTGA
CATTTCCAGTATTACTTAGTATTTTTTGTGTTATGTTGGCGAGTAGGCTTTTCATATTTTTATACAATGAATATAGCCCTTGGGTGTATCTCTAACAGAATGT
GCTCATCGACATTTACTAGCACAAACTTTCAGTTTTGATTTCATGCTCTACTTTAACTGACCCATATTATTCTCTTAATCGCAAGACTTAGTGAGAAAGAGAA
AGCAGTTCTGACCATCGGAAAGGCCTGCCAGCTGCTTTGATTATAGGGGCTGCCGGTCCAGGACGNTGGCACAAACCCACACACACAGAGACAGGCCTCGGAT
GGAGAAGGANAAAGACAACAAAACAATACATTGTTAGGCTACAGGAAGGAAACTACGC
>‘990729A-056.scf” came from CONTIG 50 at offset 0;
“E:\SEQUENCE\export\EST_db\990729a\990729A-056.scf’ (56>450)
GCACGAGGCTTCACCGTCTCTGCCATGCACGGGAAACATGGGACCAAAAAGAACGAGACGTTATCATGAGGGAGTTCCGCTCTGGCTCTAGCAGAGTATTGAT
TACCACTGACCTACTGGCCAGAGGTATTGATGTACAGGAAGTTTCCTAGTCATCAACTATGACCTCCCCACCAATAGGGAAAACTATATCCACAGAATTGGGC
GTGGCGGGACGTTTCGGCCGTAGGGTGTGGCTATTAACATGGTGACAGAGAGGACAGAGGACTCTCGAGACTCGAAACCTCTACACACCTCCTTGGGGAATGC
CCTCATGTTGCTGCCTCTCTGGGGGGGCTGTTGGCTCCTACCACAGCCGGCTGAAACCTGGGGGGCGAGGCGCAGGGANGGGGGA
>‘990729A-057.scf” came from CONTIG 51 at offset 0;
“E:\SEQUENCE\export\EST_db\990729a\990729A-057.scf’ (56>460)
GCACGAGGATTGATCAGAGCATTGAGCAATACAATTTAATTCACTCCCCCTCCCTUCCCCCTCTCCAAAAGATTTGGAATTTTTTTTTTTCAACACTCTTACA
CCTGTTGTGGAAAATGTGAACCTTGTAAGAAAACCAAAATAAAAATTGAAAAATAAAAACCATGAACACATTTGCAAAAAAAAAAAAAAAAAACTGGAGGGGG
GGCCCGGTACCCCGCCCTATAGTGAGTCGTATTACAATTCACTGGCCGACGCTTACAACGTCGAGACTGGGAAAACCCTGGCGTTACCCAACTTAATCGCCTT
GCAGCCATCCCCCTTTCGCGAGCTGGGCGAATAGCGAAGAGGCCCGCCCGACCGCCCTCCCACAGCTGGCAGCCGATGGGAAGGCAATGGGG
>‘990729A-058.scf” came from CONTIG 52 at offset 0;
“E:\SEQUENCE\export\EST_db\990729a\990729A-058.scf’ (56>590)
GCACGAGGATCAGCCGCTCGGTGACCGTCACAGGGCCTACCTGATGCAGAAGATGAACCTGTCGCTCAACGATGCCTACGACTTTGTCAAGAGGAAAAAGTCC
AACATCTCACCCAACTTCAACTTTATGGGGCAGCTGCTGGACTTCGAGCGGACGCTGGGGCTGAGCAGCCCGTGTGACAACCACACCCCCAGCGAGCAGCTCT
ATTTCTCCACACCTACCAACCACAACCTGTTCCCACTCAACACGCTCGAGTCCACGTGAGGCCGGGGGCACTGGGCGATGGGCTAGCCCCTCCCGGGCCCCCA
CAGGGCCCGCCGGCAGGGCCCCAGCCTGCCGCCTCTGGCCCGAGGACCCAGACTCACCGTGCCGGGTGAGCTCCCTCAGGTCCCACACCGGCCTGCACGGCAG
ACTTTCGAGGGCCGAGCGACAGACACAGGCTTGGAGTCCTGGGCCTCTCTGNCCAGACCTCTTCTGTGAGCTCAGCGNTGCTGTTTTTAAGACACCACGACGG
TTACTTTACTTGCAGAACCC
>‘990729A-059.scf” came from CONTIG 53 at offset 0;
“E:\SEQUENCE\export\EST_db\990729a\990729A-059.scf’ (46>550)
TTGGTGCAGCGCCTCTCGTCTTGCAGGCTCTCCTAGCTTTCGCGTCGCTTCCGGAAACATGGGCCTCCGGTGTGGCTGTCTCTGATGGGGATCATCAAGTGTT
CAACGACATGAAAAGTGCGTAAGTCGAGGACACCAGAGGAAGTGAAGAAGCGCAGGAAGGCGGTGCTCTTCTGCCTGAGTGAGGACTTGAAGATTTTTATCCT
GGAGGGGGGCATGGTGATCCTGGGGGGTGTCGTGGGCCAGACGGTGACGACCCCTTTGCCCCCUGTCAAGATGCTCCAGACAGGGCTGCCGCTCGCCCTTATG
TGCACCTAGAACCCAGAAGCGGAGGGGGCCTGTGTCTTTCTCTGGCCCCGCGGGCCCCCCTAGACAATGACTTGGGCTCCAGACGCTTATAGAGCTGCGGGTC
AACTGATTACAGCCACGCTCAGGGGGCAACCCTCCCCTCCGAAACCGGGCACCTCCTTTCCGCGGAGCCTGGGCCCCCACCCTCTGGCTG
>‘990729A-060.scf” came from CONTIG 54 at offset 0;
“E:\SEQUENCE\export\EST_db\990729a\990729A-060.scf’ (30>644)
CGGATCCCCCGGCCTGCAGGAAHCGGCACGAGGGTTTTCATGACCTGCTATCGCAGCTGGATGATCAATACAGTCGCTTTTCTTTGGAGAATAATTTTTTATT
GCAACATAACATAAGGAAAAGCAAGCGTAACCTTCAGGATAATTTTCAGGAAGACCCAATACAGATGTCTATGATCATCTGTCTGTCTGAAGGAGGAAAGAAA
GATCTTGGATCATGCCCAGAGAATCAGCCAGGCGCAGTCTGGGAATATCCAGAGCACTGTAATGTTAGACAAACAGAAGGAGCTTGACAGCAAAGGCAGAAAT
GTGGAGGTTATGAGTATTGAACATGAAATCAAGACTCTAGAAGACTTGCAAGATGAATATGACTTTAAATGCAAAAACCTTGCAGAACAGAGAACATGAAACC
AATGGCGTGGCAAAGAAGCACCAGAAACAAGAACAGCCGTTACTCCACAAGATGTCCTAATGCTGGACAATGGAGAAAGGAGTTGTCCCAAAAAATAGAGTGG
TGAATGCCACTGACTACCCAGAGCCCGAAATGATGACTGGGGGGGGAACGGGACAGCGAGGCGGTTGGGGGCCCCACGCTGCCGTTAATGT
>‘990729A-061.scf” came from CONTIG 55 at offset 0;
“E:\SEQUENCE\export\EST_db\990729a\990729A-061.scf’ (47>604)
TGGTTTTTTTTTTTTTTTTTAATTGTTCAAGCTTGTTTTATTTTGCCATGTGAATTTTTAAAAAATTGATAAAACACTAGGAACTAATTCACAACTCTTTCTA
CCCTCTTTTTATGTCAAAAATACAAAATGACTATCCATTGCATATCAGGAGAAATTAAGCTTTTTACAGAACAGGTTGGAAGAGACTATAGGTGATAATTAAT
AAGATTTAAGAATATTTTCCATAAAAACAACAAATGAGAACCTCTTACATAAAATTCTAAATACATGCTAAATATATTAGGAAAACAAACATATTTTGGACAT
TGTTATACATGCCTATAAAANGAGTTGGGGCTGTTAAAAAACTAATAAAATGCTACTACCANACTATATACAAAACTCTTAAAACTAGTTTTCTCTTACATAT
GGCTCTGAATATTTATGACACAAACTATTCACGAACAGACGGGATCATTGAGATGAGCAAATTAAATTATTCCCGGAAAAAACCTCAGTTTCATTTCCTATTA
ATAAGTGNTAATACTGGCCATCGTGACGCATAAAAAG
>‘990729A-062.scf” came from CONTIG 56 at offset 0;
“E:\SEQUENCE\export\EST_db\990729a\990729A-062.scf’ (50>470)
GGAGAGTCGGTGGGATCTCTTTACGGCCTCTGGACTGGAGGCCGAGCCCCCGCCCGCGGCCGAGCCCCGCGCCCGGCGTCTCCGGCGGGGTGCTCTCCGCAGT
TTCCTGGCTTGGAAGCCATGGGAACATGGCGAGGCAGCGGTGGTTTAACGGGAAGGACGGAGACTGTTAGCCTGTGAACGAAAGCGAGAGTGAGCCGCCTCAC
GCTCCGGACCAAGAGTGATCTTGAACnGTGGCTGCTACTAGATTTTTGCCCAGACCTCCTCGTATGCTGGGGCTTCTCCATGGAGTGGGGCCAGGGTGACCGG
GGACTTGCCGCAAGTCAGACGGCTGCAGGTCACAGCAGTCCAGNCTGTCCACCTTTGAGCGCGGGCGCATGTCCTTCCCACCCCGAGACCCCGCTGGCTTGAC
CCAAAACG
>‘990729A-063.scf” came from CONTIG 57 at offset 0;
“E:\SEQUENCE\export\EST_db\990729a\990729A-063.scf’ (47>374)
TCTGGTTGCTGTTTTTTTTTTTTTTTTTTAATTTACTAAGTCTCCTTTATTTTTGTTACCAATAATAAAACATTTGGGAGAGATTTGTAAAAACCAGGCCAGC
CCAGGGCACTGAGATACTGGGACAGGGTATTTCCCATGAGCATTCCTTGGTGGGGGTCAGGCCGTAGCTCTGGCTCCATTCGGTTTGGTGGGCTGTTGCCTCG
CCCCCCTTTGCTGCCCTGTAGAACACACGGGGGGGACTGGGGGCGGGCAATGGATCCCCTTGCCGGTCCTTGCGCCCGGCTTCTGGGGTCCTCAGCTGACCTC
CCTCGTTTCGTTTCGGGGG
>‘990729A-064.scf” came from CONTIG 58 at offset 0;
“E:\SEQUENCE\export\EST_db\990729a\990729A-064.scf’ (51>548)
GGACTGTGGAGAAGGGCAAGGACCCCAAGGGCCCCGGAGGGTGAGGAGTCGGAGGAGGAGTGGGCTCCAGTAGAGAAAATCAAGTGCCCCCCCATCGGGATGG
AGTCACACCGCATTGAGGACAACCAGATCCGGGCCTCCTCCATGCTGCGCCACGGCCTGGGTGCACAGCGAGGCCGGGTCAACATGCAGGCTGGCGACACTGA
GGACGACTACTACGATGGGGCGTGGTGTGCTGAGGATGACTCCCAACCCAGGGATAGAGGGGACACGAGAAGACCACAATTCACAGCGCCACACCAGGCCGGA
CTCCGCATCATGACACUGGACCCCTCTCGGGGCTCACACGACGCAGCGGGGGATGACACACGCTACAGAAAGCTTCAGGGACGGATAGACCGCCGGCGACGAC
TCCGGCGNAGGCCGTCATCCACACCTCCCGAAGAGCGGCGCGCGAGGCGGGGCCGGCCTGCCGTCACGCGAGGGGACCGGCCGCT
>‘990729A-065.scf” came from CONTIG 59 at offset 0;
“E:\SEQUENCE\export\EST_db\990729a\990729A-065.scf’ (48>589)
TAGGCAACTCTATCAAACGCTTACTAATTGGAAGCCTCTTCGCAGGATACATCATTTCCAACAATATTCCTCCAACAACAATTCCCCAAATAACTATGCCCTA
CTACCTAAAAACAACAGGCCTAATTGTTACAATCCTAGGCTTCATCTTAGCCCTAGAAATCAGTAATATAACTTCTAAAAATATCACTACCCCTCAAACGCCT
TCAAGTTCTCAACCTTGCTAGGGTATTTCCCCACAATTATACATCGCCTAGCTCCATACATAAATTTATCAATAGCCAAAAATCAGCATCCTGCCTCTAGACC
TATCTGACTGGAGCCATCCTACCMAAAACCATCTCACTCGCCCAATAAAAGCTGTACCTGGNCACAAGCAAAAGACTGATCAACTTATTCCTTCCTTCTATCA
CATCCTATAGATATCTATTATTACAGAGTATTCATATACCACACACATAAAAGACACCATACATACTACAGAGCTACTGTAAGCGAGTCTTGCCTTACTAAGA
CAGATCCGTTATAATACATCCTAN
>‘990729A-066.scf” came from CONTIG 60 at offset 0;
“E:\SEQUENCE\export\EST_db\990729a\990729A-066.scf’ (44>143)
TTTTTTTGCCAGCCACTTCTACCGGCAGATTGGGAGGCGAGCGCTGGGTGTGGAACATCATTCTCACCACCAGTCTCTTCTCTGTGCCTTTCTTCCTGAC
>‘990729A-067.scf” came from CONTIG 61 at offset 0;
“E:\SEQUENCE\export\EST_db\990729a\990729A-067.scf’ (49>55)
ATGAATT
>‘990729A-068.scf” came from CONTIG 62 at offset 0;
“E:\SEQUENCE\export\EST_db\990729a\990729A-068.scf’ (57>391)
GGGCACGAGGCGGTTGTTAACCTGGGGTATGTTGGTTTCGTGGAGATGCCCATGAGTGTTTTATTTCACCTGGTGGCCAGAGCTGAAATGGAACTTATTGAAA
AGTAAGGTGTAAGCTTAGATGGAAAGAAGATCTTGGTAATAGGAGCCCATGGGTCTTTGGAAAGCCACCCTACATTTGTGTGTTCCAGAGAAAAGGGACCATG
ACAATGAGCTACCAGTGGAAAACACCTCAGCTGCAAGACAAGCTACAGGAGGCTGGTCTTGAGGTCTTGGGCTCACCCAAGCCAGAAGAGATCCCCTTTCTTG
GTTCAACGGGAACTACTGTTTAAACTG
>‘990729A-069.scf” came from CONTIG 62 at offset 2;
“E:\SEQUENCE\export\EST_db\990729a\990729A-069.scf’ (59>496)
GCACGAGGCGGATGTCAACCTGGGGAAGTTGGTCCGAGGAGATGCCCATGAGTGTTTTATTTCACCTGTGGCCAGAGCTGTAATCGAACTTCTTGAAAAGTCA
GGTGTCAGCTTAGATGGAAAGAAGATCTTGGTAATAGGAGCCCATGGGTCTTTGGAAGCCACCCTACAATGTCTGTTCCAGAGAAAAGGGTCCATGACAATGA
GCTCCCAGTGGAAAACACCTCAGCTTCAAGGCAAGCTACAGGAGGCTGATATTGTGGTCTTGGGCTCACCCAAGCCAGAAGAGATCCNCCTTTCTTGGATTCA
ACCGGGAACTACTGTTTTCAACTGTTCGCATGACTTTCTATCAGGGAAGGCTGCATGCATTTCTTCTGGCGTCCATGGTATTAGCCCCATCGCCAAGATGTGG
NTCTCCTTGCTGCTGCTCTGCGAATA
>‘990729A-070.scf” came from CONTIG 63 at offset 0;
“E:\SEQUENCE\export\EST_db\990729a\990729A-070.scf’ (55>274)
GGGCACGAGGCGTGTTTTTTTTTTTTTTTTTGTACTGCTCAACTTGGTACTTATGAAATGATCATTACCTAATGGTCCACTAAATTTACATATTCAGGAAATT
ATATATAGAATACTGCAAAAACACAGTAGAAGACTGAAGGTGGCCCGGTTCAGCTCATGAAATCCCTTCACTCCCAAGCATGTTGTCCTTTGAACTCCAAAGT
GAACTGGCTGGAC
>‘990729A-071.scf” came from CONTIG 64 at offset 0;
“E:\SEQUENCE\export\EST_db\990729a\990729A-071.scf’ (54>478)
CAAAGAGCTGCCCCCATCCGAGTTCCTGCAAGTGGGCCGAGGGCCACGTGGTCTGGGGGGTGAACGTGGTCGCTCCCCCAGCGGGGGCGGGCCGGTGCAGGGC
CGACCGGGGTTCACCAAGGATCGGCCCGGGTCAGTGGATCGGCACCCGCTGGGCCGAGGCACCACGTGGTCCGGGGCTTGATGCTGGTCCAACCTACCTAGAC
ATGACTLTTCATCCTTGTGCGGGGAGCAGGGACCTGTCCGCGGGGGATCCACACGCTGGGCCCGGGAGTTCGACATTTTACCGAGAGGTGGGCGGGGAGCCTG
TGCCCTGTGCCAGGGACGATTCCAGCAACGGCTTCCCCGTGCCCAGCATGGGCCCAGCCCCCACGNGCACCCCACCAGAACCCAAAACCGATCTGCGTNCTCA
CGCTGACCCGAGACC
>‘990729A-072.scf” came from CONTIG 65 at offset 0;
“E:\SEQUENCE\export\EST_db\990729a\990729A-072.scf’ (54>526)
TCTCGCGAGATCCGCCTCCTCAATACCAAGCGCCTGTGTGTGGCAGAGCCGGGGTGAGACGAAGAGACAATCCTTCCCAGCCGCCAGGATAATCAAGAGTTTT
GGCCGGACCTTCGAGCACACACCGAGATAGTGAGGAGCCAGACGAAAAGCACAGACTATGGCGGCTGAAACGGATTAATAAGGAACTTAGTGATTGGGCCCGC
GACCCTCCAGCACAATGTTCTGCAGGTCCAGGGGGGATGATATGTTTCATTGGCAAGCCACAATTTGGGACCTAAGACAGCCCATATAGGCGGTGTATTCTTT
TGACATTCATTTCCTACGACACCCCTCAACCCTAGNTGCATGACAACAGAATTANATCCAATATAACGNAAGGCGCATTGTCTGAATTCAGACCAGTGTTCCT
GTTACATFFTAAGTCTTTTCATTGTTATGTTGGACCAACCAAGACCCTAGGCGGATGCGACT
>‘990809A-089.scf” came from CONTIG 1 at offset 0;
“c:\export\EG_DB\990820a\990820A-089.scf’ (60>576)
GCACGAGGCAGAGAGTCATAAAAGGTTTTTAGCAGAGGAGTAGTCTTTTTAGAACAATGATTCCGACAGTAGTATATGGAAATGGGAGCAGATACAGATTTAG
GCAAGGAGGCCAGAAGACGATCTCAACAGTAGTAACACTGATAAGAGGTATTGGTGGTCTAGATTTAGAGTAAAATGCAAAACTAAGGTTAGATCTATTAAAA
TACATGACTCAAAGGAAAAATTGAAGACCTAAAATTGGCTTCAATCTATTTAAAGAAAAAAAAAAAAAAAAACTCGGGGGGGGCCCCGGACCCAATTGGCTCT
AGTGAGTCGTATACAATCACTGGCCGCCGTTTACACGGGGNGACTGGAAAACCTGCGTACCCACTTATTGCCTGCAGCACATCCCTTCGCCGCGNCGTATAGG
AAGAGCCCGCCGTCGCCCTTCACAGTGGCAGCGATGGGATGGAGATGTAGGTATTTTGTAAATGCGTTATTTGTTATAGTCTTTTTACAATGCGACGCAATCT
TTA
>‘990809A-065.scf” came from CONTIG 2 at offset 0;
“c:\export\EG_DB\990820a\990820A-065.scf’ (56>544)
TTTATTTAAACCTAATTCCCCCCTTTTTGCCTTTAAAGAGCCAATATCGTTTAAGGTTTGTTTGGTTTTTTTTTTTTTTTTCCAAAATGAGAGGGAAATGGGC
ATTTTCTGTGTTGGGATGGGACTTGTAGCTTTGACTGCTCCGGGCCGGGCATCTCAGGGACTTCGCTGCTCTTTTTTCCTAGGAACGCCCCAGGTTTCCGGTG
GGAAGACTCTCGGGGCCCGGGAATTCCTCGGGGCCGCCCGGCCAGCCGGCGCGCGCCAGTGCCTCCGCCGCCCACGCGAGTCTGCTCGACAGAGGCGGAGCGT
GGCCGAGCTCAGGCTGCAGCTGCGGCAGATGCGACAGCTCCAGTACGGCGGCGTCCCCGGGNCGGNGNCCCTGACCGGGCTCTCCCCACCGCTGCCTCCGCAC
CCCGCCCACACATGTGCGGTGGCTGCTTGCGCATCTTGTACGATGATGAGGCGGGGCAGGCGGCCACCGGCTCTCCTA
>‘990809A-017.scf” came from CONTIG 3 at offset 0;
“C:\export\EG_DB\990820a\990820A-017.scf’ (451>525)
AAATATATATATTTTTTTTTTTTTTTTTTTTTTTTTTTTTTTTTTTTTTTTTTTTTTTTTTTTTTTTCTTTTTTT
>‘990809A-043.scf” came from CONTIG 3 at offset 32;
“C:\export\EG_DB\990820a\990820A-043.scf’ (50>572)
TGGTTTTTTTTTTTTTTTTTTTTTTTTTTTTTTTTCATTTCCCATTTATTTTTGGTTTTGGGGGCAATGTGATGTTTTAAAAATGAAAAAAAGAAGAAAGATG
AACACAATATAAATTTGAATTGTAAAATAGGACAAAATCAAGAAGTGGGGAAGCAGAAGGGGGAAAGGAGGGATGGTTGCCAAAAGATTGAAGGGGCGCTGGG
GCCTGTGGGGGGAATGACGTGTGGGGGGCAGCACATACCTTTCCATATAAGGACAAAAGGGGTTGATCCGTCTCATTTTTTTTTGTTAAGAAAAGAGGGGGGG
GGGGGGGCTTTCTGGGGACACTTAAGAACCAAGTTCTTTCTTGGTTTCTTGACCCCCTTTTCAAACAGAGAAGGCGGGATTCACGCTTGTAGAAGAGATAAGA
GGAAGAGAAGAATCCGACTGAGGGGGGGGCAGAACTGGAGGTAAGGCTAGGAAGAAGGGAGAGATGAAAGAAATAAAGTCACACCGCCAGCGGGGGACGGGAA
CCGCACCAGA
>‘990809A-061.scf” came from CONTIG 4 at offset 0;
“C:\export\EG_DB\990820a\990820A-061.scf’ (319>477)
CACCACCTCAACATCTTTTTTCTTTTTTCTTCAAAAATCTTGTGTTTTTTCTTTACCCCTTATATTTATTTCCTTAGGATGTGTGGGTGTTTTTTTTTTTTTT
TTTTCTATTTATCCCCGCCCTTCTTCTTCATCTTTTTCCTTTTTCTTCCCATCCT
>‘990809A-095.scf” came from CONTIG 5 at offset 0;
“C:\export\EG_DB\990820a\990820A-095.scf’ (62>535)
GCACGAGGCTGCCCTCTGGTCTGCCTCAGATCCAAGATGTCCAGTCCCCTGGAGCAGGCGCTGGCTGTGATGGTCGCCACCTTCCACAAGTACTCTGGCCAAG
AGGGCGACAAGTTCAAGCTGAGTAAGGGGGAGATGAAGGAACTTCTGCACAAGGAGCTGCCCAGCTTTGTGGGGGAGAAGGTGGATGAGGAGGGCCTGAAGAA
GCTGATGGGTGATCTGGATGAGAACAGAGACCAGCAGGGGGACTTCCAGGAGTACGCCGTCTTCCTGCCCTCACACGATCATGTGCAATGATTTTTTTTCAGG
GCTCCCCAGCACGGGCCTGATGCAAGCTTGGGCTCCCTGCTTGGGTCTTTCGCCCAGAAGACTCTTTATTTTTTTTTTTACTCATAACTTTTTGGTGTTGCAG
GAAAAAAAAAAAAAAAAAAAAAAAAAAAAAAAAAAAAAAAAAAACTGGGGCCGGCCCCATCG
>‘990809A-056.scf” came from CONTIG 6 at offset 0;
“C:\export\EG_DB\990820a\990820A-056.scf’ (53>609)
TGGGTTTTTTTTTTTTTTTTTTTTTATTTTAAATATACAGTAGGAAACATTTATTTTAACACTTCTAAAAGATATTTCTCCATGCCTGATGATTTGATATAAA
AATCAAACCCATCATACTTTCCCCATCAGTCTCTCTACATCAAGGGCAATCAGAAATTGTACAACATGAATATCTGCTTCTGAAACAAAAATTACAAATTAAG
TGATAACAAAAATACACAAATCAACTGGACCCTAAACAAATCTAATGAATTCCCCCCCCAGCCCCTCAATCACCTAGAGCTTCTTTCTTCAGCCTCATTCTGC
TCCTTTTCCTTTCTTTGCTTGGCCATGAACTTCTCTGTATTTTGCTTATGACGTGTACTCTTGCGGGATTTGTCTTGCTTTTCTCTGAGTAGIAAGAGGGACA
ATGGACAGAGGATCCAGCCTCGNACAGAAGTCTAATCTTAGGACACAGGGCCACGACTCCTGAGGCCTCATCTTTGCGTCTGGTGGTCTGATCTTATCAATCT
CACTATGGAGGCTTCTCTTCTGTTCACGACACTTTCGAG
>‘990809A-027.scf” came from CONTIG 7 at offset 0;
“C:\export\EG_DB\990820a\990820A-027.scf’ (53>464)
TTTTGTTTCCCAAATGTCTCTCCAGACTGTCTACATCTCCGCTACAATCACCTGAGTACCAACTGTGTCATCTCTCACCATTTGTATCTGTCTCCGTGCAATC
CATCCTCCACTAGAGCCAGAATGTTCTTTCCAAGATACAGATGTCAGCTGGCCATCCTCCATCATAAACCCTTCAATGGCACTCACTGCTCAGTAAAGACATT
CTGCCTFFGTGGCCCCTGCTACCCTCTGTGACCTTCTCTGCCACCATGGCTCTCCCAGCTCACTCCCAGAGTCTCAGAGTTCATGTTCCATGTGACCTCCTGC
ACAGAACCTCTGCCAGTGCTCTTTATCCCCTCTTCACCTGCAATCCAAGCGCTACTTCTTCAGAAAAAAAAAAAAAACAACCCTTACGGCGNTGCGC
>‘990809A-079.scf” came from CONTIG 8 at offset 0;
“C:\export\EG_DB\990820a\990820A-079.scf’ (58>614)
GCACGAGGCGCTGACTATTCTCAACCAACCATAAAGATATTGGTACCCTTTATCTACTATTTGGTGCTTGGGCCGGTATAGTAGGAACAGCTCTAAGCCTTCT
AATTCGCGCTGAATTAGGCCAACCCGGAACTCTGCTCGGAGACGACCAAATCTACAACGTAGTFPGCAACCGCACACGCATTGTAATAATCTTCTTCATAGTA
ATAGCAATCATAATTGGAGGATTCGGTAACTGACTTGTTCCCCTAATAATTGGTGCTCCCGATATAGCATTTCCCCGAATAAATAATATAAGCTTCTGACTCC
TCCGTCGCTGATTCCTACTACTCCTCGCATCCTCTATAGTTGAAGCTGGGGCAGGAACAGGCTGNACCGNGTACCCTCCCTTTAGCAGCCAACCTAGCCATGC
AGGAGCTCATAGATCTACCATTTCTCTTACCTTACAGAGTTTCTCATTTAGAGCATCACTCATTCACATTATCACTAAGCCCCGCATGCCATACCACCCTTGT
TGAGAACGAATATACGCGACTCTCTACTTGTCTTTTGCGCGC
>‘990809A-021.scf” came from CONTIG 8 at offset 9;
“C:\export\EG_DB\990820a\990820A-021.scf’ (61>617)
GCACGAGGCTCAACCAACCATAAAGATATTGGTTACCCTTTATCTACTATTTGGGTGCTTGGGCCGGGATAGGAGGAACAGCTCTAAACCTTCTAATTCGCGC
TGAATTAGGCCAACCCGGAACTCTGCTCGGAGACGACCAAATCTACAACGTAGGTGTAACCGCACACGCATTTGTAATAATCTTCTTCATAGTAATACCAATC
ATAATTGGGAGGATTCGGTAACTGACTTCCCCTAATAATTGGTGCTCCCGATATAGCATTTCCCCGAATAAATAATATAAGCTTCTGACTCCTCCCTCCCTCA
TTCCTACTACTCCTCGCATCGTCTATAGTTGAAGCTGGGGCAGGAACAGGCTNGACCGCGTACCCTCCCTTAGCAGCAACCTAGCCATGCAGGAGCTCATAGA
TCTACCTTTTTCTTTCACTAGCAGAGTTTCTGATTTTAGAGCATCACTCATACACAATATAACAAAGCCCCGCATGCCATACAACCCTCTGTGTGATCGTATA
TACGCGACTCTCTCTTGCTCTGTTTGAGCGGTACAGCTTA
>‘990809A-006.scf” came from CONTIG 9 at offset 9;
“C:\export\EG_DB\990820a\990820A-006.scf’ (51>493)
TTGGAATTAGGCACGAGGACCCACATACCTTCAAAAGAAAACGAGGTGCTGACCTTGGCTGTGCTCTCTTCCTGACGGGGAGCCAGGCTCGGCATTTTTGGCA
GCAAGATGACCCCCAGTCATCCTGGGATCGGGTGAAGGATTTTTGCCACCGTGTATGTGGAAGCAATCAAGGATAGAGGCAGAGACTATGTGGCCCAATTCGA
AGCCTCCGCTTTGGGAAAACAGCTCAACCTGAAACTCCTGGACAACTGGGACACCCTGGCCAGCACGTTTGTCCAAAAGCGTGAACAGCTGGCCCAATGACCC
AGAGTTCTGGGACAACCTGGAAAAGAGACCGCGTCGCTGAGGCAGAGATGCCAGGACTGAAGGAGTGAAGAGAGGGCAGCCTACTGACGGTCAGAGAAGGACG
AGAGTGAGATTACGCGAAGGGGGCGTGGCGGAGT
>‘990809A-057.scf” came from CONTIG 9 at offset 17;
“C:\export\EG_DB\990820a\990820A-057.scf’ (54>482)
GACAGGATCCCTTCAAGAGAAGCCGGGTGCTGACCTTGGCTGTGCTCTTCCTGACGGGGAGCCAGGCTCGGCATTTCTGGCAGCAAGATGACCCCCAGTCATC
CTGGGATCGGGTGAAGGATTTTGCCACCGTGTATGTGGAAGCAATCAAGGATAGTGGCAGAGACTATGTGGCCCAATTCGAAGCCTCCGCTTTGGGAAAACAG
CTCAACCTGAAACTCCTGGACAACTGGGACACCCTGGCCAGCACGTTTGTCCAAAGTGCGTGAACAGCTGGGCCCAGTGACCCAGGAGTTCTGGGACAACCTG
GAAAGGAGACCGCGGTCGCTGAGCAGGAGATGCACAGGACCTGGAGGAGTGAAGCAGAAGGCAGCCCTCCTGACGAGTTCCAGAGAGGGGACGAGAGTGGAGA
CTACCGCGAGGTGGNGCG
>‘990809A-066.scf” came from CONTIG 10 at offset 0;
“C:\export\EG_DB\990820a\990820A-066.scf’ (62>596)
GCACGATGGCACTTCCTGGCACCTCTAGGTATTGCTGGTCAGGGGGGTGTGGGCGGCCTGCCTGGCCAGAGCAGGAGAAAGAGGCTTCCCTGGGCTTCGTGGG
CCGTCTGGTGAACCCGGCAAACAAGGTGCTTTGGGAGCAAGGGGTGAACGTGGCCCCCCTGGCTCCCATGGGTCCCCCCTTGGATTTGGCTGTGTCCCCCCTG
GCGAGTTCTGGACATTGTAGGGATCTCCCTGTGTGCTGAATGCATCCCCTGGACGAATAGTTCTCCCCTGGCGCCAAGGGTGACCTCGGCTGAGACCGTCCCT
TCTGGTCCTCTTGTGCTTTTGGGGCTCCTGTGTCCCCGTTCCTGTTGTCCTGCCTGGTATGATTGTTTTCGTGTTTTCCGTGCCTTGTGTTCTGTGTTTCCTT
TTCCCCTTTGGTCCGTGTCCCGCTTGCCCTAGTCCCCTGGTGAATGTGTGACAGGTACAGTCAAAAGCTTAAGGCCACCTTTTCTCTGCTCATGTCCCCCCCC
TCCGTTTCTGGTGTCAGTCT
>‘990809A-093.scf” came from CONTIG 10 at offset 5;
“C:\export\EG_DB\990820a\990820A-093.scf’ (56>547)
CTGGCACTCCTGGACCTCAAGGTATTGCTGGACAGGGTGGTGTGGGGCGGCCTGCCTGGTCAGAGAGGGAGAAAGAGGCTTCCCTGGGTACTTCCTGGGCCCC
TCTGGCGAACCCGGCAAACAAGGTCCTTCTGGAGCAAGTGGGGAACGTGGCCCCCCTGGTCCCATGGGCCCCCCTGGATTTGGCTGGACCCCCTGGCGAGTCT
GGACGTGAGGGAGCTCCTGGGGCTGAAGGATCCCCTGCCGAGATGGCTCTCCTGGCGCCAGGGTGACCGNGGNGAGACCGGCCTGCTGACCTNCTGTGCTGGT
GCGCTCCCGGGCCCCCGCCCTGTCGGACCTGCGCAGAGGNGATGTGGGAGACGGGCTGTTGTCTGCTGTCCCATGCCCGTGTGCCCGGCCCCGTGACCCAGGC
CCGGNGACAGGNGGACAGGACAGGCACGAGAATAAGTNACGGCTGTTGTCTCAGAACCCGCCTTCGCTTCTGGAGAGACCT
>‘990809A-037.scf” came from CONTIG 11 at offset 0;
“C:\export\EG_DB\990820a\990820A-037.scf’ (60>606)
GCACGAGGCCCGGCCCTCCTGGACCCCCTGGGTCCCCCAGATCCTCCCAGCGGCGGCGTACGACTTGGAGCTTCCTGCCCCAACCACCTCAAGAGAAGGGTCA
CGATGGGGGGCCGCTACTACCGGGGCTGATGATGCCAATGTGGTCCGTGACCGCGACCTCGAGGTGGACACCACCCTCAAGAGCCTGAGCCAGCAGATCGAGA
ACATCCGGAGCCCTGAAGGGAGACGCAAGAACCCCGCCCGCACCTGCCGCGACCTCAAGATGTGCCACTCTGACTGGAAGAGCGGAGAATACTGGGTTGACCC
CAACCAAAGGTGCAACCTGGATGCCATTAAGGGGTTTTGCACATGGGACCCGGGAGAGCTGTGNTACCCCACTCAGCCAGGTGCCCAGAGTACGTTATTCATC
AGAACCCAGAAAAAGGACGCGGGGCGGGGAGATGACGCGGTGCATCGGGTTGGGGCAGGGTCGTGCTGCGTGGGCTCACTGCTTCTGGCTGTGGCCCGCGCTG
CAACATACTACCTCAGAGGGGGCTCTGTCCGG
>‘990809A-092.scf” came from CONTIG 12 at offset 0;
“C:\export\EG_DB\990820a\990820A-092.scf’ (61>533)
GCACGAGGCAGAGGTCTTCCTGGCTTAAAGGGACACAATGGGTTGCAAGGTCTCCCGGGTCTTGCTGGTCATCATGGCGATCAAGGTGCTCCCGGGGCTGTGG
GTTCCCGCTGGTCCCAGGGGCCCTGCTGGTCCTTCTGGCCCCGCTGGCAAAGACGGGCGCATTGGACAGCCTGGTGCAGGCGGACCTGCTGGCATTCGTGGCT
CTCAGGGAGCCAAGGGCCTGCTGGCCCTCCTGGGCCCCCTGGCCCTCCTGGAGCCCCTGGCCCAAGGGNGGGGGTACGAGGTTGGTTTGATGGAGACTTTCTA
CAGGGCGCCAGCCCGCTCACAACTTCTTCAACCCAGGATATGAAGTGTGCTCTCTGATATCTCACAACAATGAGACCTTCTCTCAAGGATTAGAGAACCACCG
CCAGCGGACTGAACCACCCCCACAGACAGGGTCTCTGTLTGACTACAGAGACTTGAGCTTAAA
>‘990809A-013.scf” came from CONTIG 13 at offset 0;
“C:\export\EG_DB\990820a\990820A-013.scf’ (55>581)
CTTTACCCAGCCTATCTCAGAAGTTGTAGATGAAGTAATTCAGAATTGTCCTATCGATGTCAGACGTCCTCTCTACAAGAATATTGTGCCTCTCTGGAGGTTC
AACCATGTTCAGGGACTTTGGACGTCGGTTGCAAAGAGATTTGAAAAGAACTGTAGATGCCAGGCTGAAATTAAAGAGGAATTGAGGGGTGGTAGATTGAAGC
CAAAACCTATTGATGTACAAGCCATTACACATCACATGCAACGATACGCAGCTTGGTTTGGAGGATCTATGCTGGCTTCCACACCTGCGTTCTACCAAGCATG
CCACACCAAAAAGGATTATGAAGAAATTGGACCTAGCTTCGTCGCCACATCCAGCGCTTGGGTCATGTCGCAAAATGGCTTCATAGTTTGGGGTAGGGGGGGA
GGAGAAAAGACTTCTGTTACCTGCTGCTGGTGGCTGCTGGCACCTGACTGATCATAGACAACATCATATCAGATATTTATAGATACACAGCGAAGAAGAGGCA
AAGATAGNGTTT
>‘990809A-010.scf” came from CONTIG 14 at offset 0;
“C:\export\EG_DB\990820a\990820A-010.scf’ (47>589)
TTGTTAAAGTGGCCTACAGCACCGAGGGCGCCAATTCCAGTTCATCCAGGTTGCAGGGCGGGCAGGAGATAAGATATTTATAGGTAATGTGAACAACAGCGGC
CTGAAGATTAACCTGTTTGATACCCCCTTGGAGACGCAGGATGTGAGACTGGTACCCATCATCTGCCACCGGGGCTGCACCCTCCGCTTTGAACTCCTTGGCT
GGGGAGTTGAATGGATGCACTGAACCCCTAGGCCTGAAGGATAATACCATCCCCAACAAGCAGATCACAGCCTCCAGCTACTACAAAACCTGGGGCCTGAGTG
CCTTTAGCTGGTTTCCCTACTACGCACGACTGGATATCAGGGCAGTTCAACGCCTGACCGNCCACACCAACAGGCCTCTGAGTGCTGAGATGACCTGGCTCCA
GAGCGGTCACGGCATATCACCAGGGCCCGGACTTGCCCATCATTGTGCTGCTCAGGGGCTTGNGTGTGNGGACCGCTGGACAGACCGGGCCCAGAGAGATTTC
TGTACTGACATATCCAAAAGAAATTGAC
>‘990809A-002.scf” came from CONTIG 15 at offset 0;
“C:\export\EG_DB\990820a\990820A-002.scf’ (52>545)
TTGTTTTGGTTTCTTTGAATTCCTAGAGACACAGATTCTGATAGAGAAAATTATATTAAGTTATAGAAAACTTTACAGGATAATAGCTTTATATTCATTGCAC
ACTTACCTTGAATAGTCATGGCGTTATCATAGTATCATTAGTATAGTTATCTGTATTCATTAATTGGCATTTTATGGGTTTCAGGTAGACATGACATGACATG
ACTCTAACAAAATTAAAAGAAGTAGGGCTTGTTTTACGAATTATAGTTTTATTCCTTCUGGTATATAAGAATTTGTTAAGCCAGCATATTTAGATTTATTTGT
GCAGATGCTGNTAATGTCTGGATAACTATCTTTACTTTATGGGGGGTAAATATGTGTTTTGGGTGATGTGTGGCAGCCACTGGTATGTGTGTATCTGGTCCCA
TTTACAGTAGACACATTCTCTGTTTTGTACTTCACAGCTAAATTTTTGGTTATTTCTTACCACTGCTNCCANATTA
>‘990809A-012.scf” came from CONTIG 16 at offset 0;
“C:\export\EG_DB\990820a\990820A-012.scf’ (54>607)
TTAACACCTACAAACCTTCCAACTCCCCACTCTACGCAAAAACAGCTATCTCATACGCCCTCATTACCAGCATAATTCCCACAATAATATTATAACACTGAGG
GCAAAAACTAATTATTTCAAACTGACACTGACTAACCATGCAAACTCTTAAATTATACCTCAACTTTAAAATAGACTATTTTTGAATAATATTTATCCCAATT
GCGCTATTGGTCACATGATCTATTATAGAATTCTTAATATGATTTATATACTCAGACCCCAATATTAACAAAATGTTCAAATATCUCTCCTATCCTCATTTCT
TTGCGTGTCCTTGTTACCGCAAACAACCTCTTGCGGGTTTGATTGGCTGAGGAGAGTGGGATATATCTTTTTGCTCATCGGTGATGATGCGGCGAGGAGAGCA
AAACGGAGGCGTCAGCATGCTGTTATCGGTCGGGCGTTGTTGGTGNGTAGATGTTCGTAAATTCAGTCCGGACGGCACGATCTTTCTTACCAACACCAACTCC
TGGTGTCTGCTGTTGACGGTATCGACATTGGCTC
>‘990809A-008.scf” came from CONTIG 17 at offset 0;
“C:\export\EG_DB\990820a\990820A-008.scf’ (61>623)
GCACGAGGAGAATCTTAAACCAAAATAATGCAGCAGGAGATGCCTGTGTTCCAGGTGCATTGAAAGCCAATGAGAAGTTATCTGAAGAGAGAGCACAAGATAC
ATACTGTGATGGTTCACCTTTACCTGAAGATTTTACAGAGTCTACCAAAATGAATGGCTGTGAAGAACAAAAGTGAAGAGGTAAAAGTGAAAGCTTAATTCAA
AAGACAGAAGAAAAGAAGACTGAGGATGATGAAATAACATGGGGAAGTGATGAATTGCCAATAGAAACAACAGACCATGAAGATTCCAATAAAGAGCATCCCT
TTCTGACAAATGAGGAACTCACCACACTCCCCATCATCAAAGTGCTTCCCTCCGCGAGTACACTGCTGGCCAGCTGCAGTCAGNGATGCGGTGTTGCGGGGNN
TACTCGATCAGGGATCCATCTAGGAGCTGGAGAATCTCAAGAATAAAACTTGATCATGTCTATTGACAGACGAGGAAACAGAGGAGAACGAATAAATATACGC
CCTGACGCTCGGNGCCTCTGGGTGAGGGGNTTTCACGCGCTC
>‘990809A-048.scf” came from CONTIG 18 at offset 0;
“C:\export\EG_DB\990820a\990820A-008.scf’ (62>553)
GCACGAGGGGGGGCTCAGTCCGCAACCGCCGCCGCCGCGCCTCCGTATCGGTGCTGGGAGGGGCCGCCGCCGAGACAGCCGTGCGGGCGAGCATCCCCAGGCA
GCACATTAAGAGTGGGTGATAGCGGTGTCCCGCCCTCACCGATTACATCCCGTTCTGCACATATGCACTGGGAGGGCCTTCCAGCTATAAAGTAGGCACCATG
GCTGAGAAGGTCGACTGCCACTACTGCAGAGACAACCTGCGAGGGGAAGAAGTAACGTGCAGAAAGACGGCCACCACTGCTGCCTCAAGTGCTTCGACTAGTT
CTGCGCCAACAGNTGTGTGGCAGTGCCCGCAGCCCATCGGCGCCGACTCCTAGGAGTGCACTACAGGAACCGCTACTTGCACCAACCTGCTTGGGCTGTTTTA
GTGCTCCGCCCTTTGGCAGAGAGACTTCGTGACAAGAAACAGATCCTGGCACAGGCCCCTCGGAGACAACCCAGGCTGGCT
>‘990809A-049.scf” came from CONTIG 19 at offset 0;
“C:\export\EG_DB\990820a\990820A-049.scf’ (62>438)
GCACGAGGGATTAAAGGTCTGGAAACACATAAATGGATTGCAATGAACTTTCCAGATGTAGTAGTTCATGGGGATTCTCACGGCAACAGTCGACTCATTGGAA
AAGACCCTGAGACTGGGAAATATTCAAGGCAGAAGGAGAAGAGGGGGACAGAGGATGGAGATGGGGGGGAGGCATCACCGAGTCAATGGATATGAACTTGGGC
AGACTCCGGAGATGGCAAACTCCGACCGTCTGCAGGCCATGGGGTGGCGAGAGGCGGCACATTTGGGGACGGACAACACCAACAAAGATATTTGGAGCCACYT
AAATTTTAATGATGCTCAGGTCATTAGTATGTGCTAAKTTGAGGTCCCAGTTACCCAATGGCGGGG
>‘990809A-009.scf” came from CONTIG 20 at offset 0;
“C:\export\EG_DB\990820a\990820A-009.scf’ (51>605)
TATGAGAGCAGCACCTTCCCCCTCCTCCTTTCCACACCTGCAAACTCTTTTGCTTGGGCTGAATATTTAGTGTAATTACATCTCAGCTTTGAGGGGCTCCAGA
GGCAAATCCCCGGATTAAAAGGTTCCTCGGTTGTGAAAATATACAAGAGAAATCATGAAGGCAACTATCATCTTTCTCTTGGTTGCACAAGTTTCCTGGGCTG
GACCATTTCAACAGAAAGGCTTATTPGACTTTATGCTGGAAGATGAGGCTTCTGGGATAGGCCCGGAAGAGCACTTTCCTGAAGATCCTGAAATAGAGCCTAT
GGGCCCAGTCTGCCCCTTCCGCTGTCAGTGCCATCTGCGAGTTGTCCAGTGTTCTGATCTGGGTCTGGAAAATACCAAAAGACCTCCTCCGCTACTGCGCTGC
GGACCGCAAAACACAAAATACTGAGACAAAGATGAGACTTAGAACTGAGACCTCTAGACTGTTCNATCACAACAAATAGCAAACAGCCTGGGCUGCTCTTGGT
GAATGGACACTATCTTCCAGATAACGAGGATGCAGAAAA
>‘990809A-028.scf” came from CONTIG 21 at offset 0;
“C:\export\EG_DB\990820a\990820A-028.scf’ (61>564)
GCACGAGGCAACCACGTGCTGAAGGGCAAGGACGCCAGCTGCCTGGCAGAACAGTGGGTCTGGCAAGAAGGGGGGACACAGGCCGCCCTGGGGGAGCAAGAAA
TCCAAGAAGAAGAGCGGCCGGGCCAAGGGCTTGGGCGGGGGCAGCAGGAAACAGAGGAAGGAGCTGGGCGACCTCGACGGAGACCCCAGCCCCGAGGAGGACG
AGGGCATCCAGAAGGCUCCCCGCTCACACACAGCCCCCCTGACGAGCTCTGAGCCTAACCCAGTGGCTTTTGCTGACTGAGAGCCTGAGCTGGGAGCCCAGGT
GTCTGGGCCCCCGGGCTGCACAGATGGAGCAGCGCAGGCCTCAGCGGTGCCTGCCTGGCCGTGCCGCCTCCTGCCGTGGAACTCCAGCCTGGGGNAACTFFNA
TCAGTTCGGGCAGGCGGCTGCCTTTCCTCTGGCCAGCGTCTTCCTGACCAACTCAGCGGGCCCAGATTCAGATGCCAGACTCATGTGACTGG
>‘990809A-024.scf” came from CONTIG 22 at offset 0;
“C:\export\EG_DB\990820a\990820A-024.scf’ (53>501)
TTTCAGCCATCCCTTGGGTTTGAAGACAAGCTCACGAACTCCCCACACGCCCACCACCCCCCTACCCAACTGGGCAGCCCTTCAACGAGGATGGTATCGAGGG
ACTGAAACATCTTACCTGTTCCTGTCTGTCTGGCCGCAGGGTCTAGGAGCCCCCAGGACACAGCATGAGTGGGCCTTGGACCGCTACCCCGCTATGGGCCTGC
AGACTTGGTTCCAAAGGCCTGGGCGGACAGACCGCCCAGTCACCACCTTCACCCTAGCTTGGCCACCCCAGGGCGACAAAGAGCAGCAGGGGGCGGGGGCAGC
CGCGCGGACAAAGCCGGATTTCCTGGTCGCGGCTGGCTCTGTTTCCCTGCGCTCCCCCCCCGCCGTGTGTTCCGCCGAGCTGACGTCTAACGGGGAGGGTAGA
AGGACAACCCGCAAGGTGTGGGATTGAAGGAATGTGGC
>‘990809A-020.scf” came from CONTIG 23 at offset 0;
“C:\export\EG_DB\990820a\990820A-020.scf’ (58>610)
GCACGAGGGTCAGTGTTAGCATCAACACCACGCTCCCATCTACCATTGAGCCTTGCATCTTGCCATCCCCATGGCAAGGAAGCCTTTCTCTGCTCAGATTCCT
CCTACCCTTGAAGACTTAGCTCAAAATGCAACACCTGTATCTGCTTCAGCTCATCCTATCTCTCAACTCCCTTATATGTTTGTACTTCTTACTTGAATACTTA
ATCCTATATGCTGGTGAGGGTTACTGAGTCTCTTCTATGTCTCAGTCATGTCTTATCAACAAAGCTGCAAACTTACCAAGAGACAGATCACTTCCACTCCCCA
GGGTGCCTATCACAGGTTTTAGTAGCAGAGAGGAGCTCAAATCCACTGTTGATTTCATCTTTGTTCCATTTGCTATTATCAGTTCATTTAACAAAGAGNAGTA
TGCTCTATACCTGCTTTTCAAAAGAAACTAAATCAGAAATATTATAAACAGAGAAGGTCATCTGTTGCTCTTGGCAGAGCCTGACAGNCNTAAACGAAAGCTC
ANGTCCCAGGAACATGGCCACGGTCGGGGGAGCGTC
T
>‘990809A-025.scf” came from CONTIG 24 at offset 0;
“C:\export\EG_DB\990820a\990820A-025.scf’ (55>597)
GGAAGATGTATGGAGACATGGCCTAAAGCCAGAGACAGGGAGAACAGGTGAACATTTTAGGGTGTCACTTGAATCGATTCACATCTCATTTTTGTGTACACGT
GATTTCAGGGGCACAAGTTATTTAAATCTGTGCTTCTAACTGGGGAAAAGAAAAATTCCCACCAAATTCAAAATACTGTGCCATGTGATATTCAAACCAATAG
TCCGCCAACCCCAGACACTGGTTTGAAGAAATTGAGACTTGATCATAGGACTGTATTAGTGCACAGCGCCAGCATGTATGCTAGGAGCAGGGGAGGAGGGCAG
CAGAAAGCCTT7GTATCTTTGGGGGGGNGGAGTGACTGGTTTTGGATGTGACTGAAAAGAAAACTTAGCATGCTCCTGTCTGCCTTAGCTCCAGCACGCCGGT
GTGCGCCCCACCTCAGAGCGAGCAGTCGCTCTAGCAGACACAATCACTTTGACTTTGATCAGACATGTCAGAATAGATCTGCTITAACGACGCCGGCAGCGAT
GCCCACAGCAATNTTTTGTACGAATTGGGNN
>‘990809A-026.scf” came from CONTIG 25 at offset 0;
“C:\export\EG_DB\990820a\990820A-026.scf’ (57>601)
AGGAACGAGGCCCAGCTCTATGTAGGCAGGGCGGGGTGGAGCTCACCTGCTCCCATGCTCTGGATGGACAGCAACTACTCTACGGGGACCAGGGGCACAAGAC
TCAGCAACTGGACCACCAGGCACCCCCCCAACCCAGAGGGGCTGGAGTFFGGGTCCTGAGGGTTCCCAGGACCAACGGCTGCAAAAACCGGATGTTACAGGAA
GGAGGCCTGCCTCCTCAGGGCCTCAGATTCTGAAATGCCCAGAGGGAGAGGGTGTCAAGCTGCGCTGTGAGCTGGGGGCACGGACCGGGAGAGAGCGCGAATC
TGCAACTGGTTGCGCGTGTGGGCCAAAACCCTCTACAGCGGGAGCACCAGCCTTTAGACGGAGAGTGAGGCGGGATGAGCCCCGAAAGCACTATAAAACTGCT
CGACGGTTCCAGAGAGCTGCAGGGGCCAGGGGCAAGGACAAGGAGAGCAGCCCGGTGCTCTTGATCTATCTATCTGCCATGCCTCCGTCCGTCGCCTTTTACT
TAAAATGATTTTAAGCTCGTCGCGGCGNCT
>‘990809A-022.scf” came from CONTIG 26 at offset 0;
“C:\export\EG_DB\990820a\990820A-022.scf’ (60>522)
GCACGAGGCTCCCTGTGGATCCTGTTCCACTTCCTGACGGGGCAAGGCAGGTCAGGAAGGTGTAGACCATCCTCAGGAAAGAGCCAAGGCCCAGGAAGTCCTC
CAAGCCATCCGGGGGCTTCGTCCGCTTCTTCGGGTGGCGAGAGGGCGCTGGCCACTTTGGGCAGATGGCCTCTGGCTCCATGCACCGGGTGGGGAGGCTGAAC
AGCGCCGTCCTTTGGGTCTGGGCCAACCACAACAAGGTGAACGCTCGCCTCGCAGGCGCCCCCAGCGAGGACCCCCAGGTCCCCAAGAGGCAGNGGCCACCCC
GCGAGCTCTGGTCCGCCTGCCACAATGAACTACGGGCACGCCTGTGTGGGACCTGCACAACATCTCAGATCTTGAGACCACTACTCCCCAGCAAAACTGCTAT
ACTTTCTTTGCTGGCCGGGGCCGGGCGGGGGAGGAGGAGGAGGAAACA
>‘990809A-003.scf” came from CONTIG 27 at offset 0;
“C:\export\EG_DB\990820a\990820A-003.scf’ (1>516)
CCTCTCGGGGGGGCCGCTTAAATGGGTCCCCGGGTTTTTGCCGGGCATTTACCCTTCAGAAAACGAGACCACCAATTCCTTTCTTTCCCGGCTGGGGACCAGC
CAGNGTTCCTGCCCACCCAGATGCTGGTGAAGATCATGGCAGATATTGCCAGAGGCATGGAGTATCTGAGTACCAAGAGATTCATACACCGGGACCTGGCTGC
TAGGAATTGCATGCTGAATGAGAACATGTCGGTGTGGTGTGGCTGACTTTGGGCTCTCCAAGAAGATCTACAACGGGGACTACTACCGGCAGGGACGCATGGC
CAAGATGCCGGNCAAGTGGATTGGCATCGAGAGCCTGGCGGACCGTGTCTATCCAGCAGAGCGATGTTGGGGCTTFGGGTGACGATGTGGAGATGGCAGCGAG
GCAACCCCTATCAGNATGGAGACAGGAGATATGATACTGGCCAGGAACGCTGAGCACCGTGACTGCTGACGACGACGCCGTGTCGTGCTGGGCTAACCCCGAC
GC
>‘990809A-001.scf” came from CONTIG 28 at offset 0;
“C:\export\EG_DB\990820a\990820A-001.scf’ (62>533)
GCACGAGGCTACCAGTATGGATTCAACCTGGTCATGTCCCATCCTCATGCTGTCAATGAGATTGCGCTGAGTCTCAACAACAAGAATCCAAGGACCAAAGCCC
TTGTCTTAGAGCTCCTGGGAGCTGTGTGTTTTGGTACGAGGAGGGCACGAAATCATTCTCGCTGCCTTTGACAATTTCAAAGAGGTGTGCAAGGAGCTGCACC
GCTTTGAGAAGCTGATGGAGTATTTCCGGAATGAGGACAGCAACATCGACTTCATGGTGGCCTGCATGCAGTTTATCAACTCGGGGGTGCACTCAGTGGAAGA
CATGAACTTTCGGGTCCACCTGCAGTTGAGTTCACAAAGCTGGGCTGGAGGAGTTCTGCAGAGTCACGCACCAGAGAGGGGAGCGCAGCGCAGATCAGGCGAC
CTGACACGGTCGTGTGGGGGGTGTGGGGTGCGGACAAAAGTGGCCTGGAAGGGAGAGTGGG
>‘990809A-004.scf” came from CONTIG 29 at offset 0;
“C:\export\EG_DB\990820a\990820A-004.scf’ (50>557)
TTGCAAGAGAGAAGACAAAGCAAATGAACTCAAAGACAAGATGGGCAAGTGGAGAGATGATGGGGAAGGATTGGGGAGCTTTGTTCTGCTGGCTCCTCTGGTC
TCAGAAATGAGAATTATAATTCCATTCAACAATGAGAATGGACAGCAGACATTGGGAAGGGAGTGAGTGACTGATTAGATGAGGGGCACAAATTGGAGATGCC
CAGAAACACTAAGTTTCCTCTTAAGTTACTGGCCTPGAATTTCAGTGGTAGCCAGCCACTAGAGTGGAGTTTTCTCTATCCTTATTCAGCTGGGAAGCTGGGG
GTTTGGGTTGAAAGAGTTGAATTTATCTAGGTTGTGACTTTGCCAGCAGATGTAGCCACCAATGAGAGAGAGCAAATATGCCAATAGAAGCTTACTTNTTCTG
NAGCCCGNGGNCTCTGGATGCTACTGTTCATAGATAACCGCAGCAGAAAGGGAACCGCCCCGTACAGGAGCTGGACGAATATGCTCCGTCAC
>‘990809A-007.scf” came from CONTIG 30 at offset 0;
“C:\export\EG_DB\990820a\990820A-007.scf’ (52>536)
TGGCCGGCTCCCGCGGCGGCTCCCGCGGCGGGCTCCCTAGGTTAGTGTGATCTCAACTCAAGAGAAAGGTGGGGCTATCATGGCATCTATCTGGGTTGGGAAA
GCGAGGGACAATAAGAGATTATGCTGGCTTAACCCATCGGGGGATGCTGAAGCGATTCGTAAGGCCATCAGAGGGAATTGGGGACCGACGAGAAAACACTGAT
CAGCATTCTGACTGAGAGGACGAATGCACAGCGGCTGCTGATTGCTAAGGAATATCAAGCACTATGTGGAAAGGAACTGAAAGATGACTTGAAGGGTGATCTC
TCTGGCCACTTCAAGCATCTCATGGTAGCCCTCGCCGCCCCACCCGCAGNGTTCGCTGCGAAACAGCTGAGAATCCATGAGGGCATGGGACAATGAGATGCAC
GATCGAAATCTACCACTGACAGCAGCAATGCAGAGACGGCATCCTCTTCACGAGAGAGGACTGAGATGGATA
>‘990809A-030.scf” came from CONTIG 31 at offset 0;
“C:\export\EG_DB\990820a\990820A-030.scf’ (48>602)
TTGTGGCAGGATGGGCAAGTGTTGCGGTCTTCGTACTGCCAGGAAGCTCCGCAGCCACCGACGAGACCAGAAGTGGGATGATAAGCAGTACAAGAAAGCCCAT
CTGGGCACAGGCCTGAAGGCCAACCCTTTTGGGGGCGCTTCTCACGCTAAGGGAATTGTGCTGGAAAAAGTAGGAGTTGAAGCCAAACAGCCAAATTCTGCAA
TCAGGAAGTGTGTCAGGGTTCAGCTAATCAAGAATGGCAAAAAGATCACTGCTTTTGTTTCCAATGATGGGTGCTTGAATTTTATTTGAGAAAATGATGAAGT
TCTGGTTGGTGGATTTGGTCGCAAAGGTTATGCTGGTGGTGACATTCCTGGGGTCGTTTTAGGTTGGAAAATAGTCATTGTTTTTTTTTGGTTATCAAAGCAA
GAGGAAAGACAAATATAAATTTGTGATGAAGACGATGAATAATTTTTTTACAAAAAAAAAAAATGGGGGGGCCGGCCCATTGCTTTTGGGTTGTTTATTATGG
CGGGTTAACGGGATGGAAACTGGGTCCACTTTTGTTCG
>‘990809A-031.scf” came from CONTIG 32 at offset 0;
“C:\export\EG_DB\990820a\990820A-031.scf’ (1>545)
ACCACGGGGGGCGTTAAACTAGGGATCCCCCGGCTGAGAATGGCACGAGGGACATTCTACCCTGGCCGCTGACTCGGGAACCTCAATAACCAAACCAGCCGAT
TCAAGGGGCCAGGGCTTTGGTGGGGCACCCGATCCCACGGGCTGACCACCACCCCCTCACGGAAGCCTCTTACGAGAACCTGCCCACCATTGCCCTGGGCAAC
ACGGACTCTCCTCTGTGCTATCGGGACATCGGCACCCCGCGCCACAACAAGGGGGCGCACTAGGGGGGGTGGGTGGCGGGTGCTCGCCCGGGGAAGTCACGCG
CATGCGGGGCACCATCTCCCCGCGCCCCGGGGGAGGGTGTCCGCCTCTCTTGTCAGAGAGCTGAGAGAAGAAAAGAAGAAGGGAGCAGAGAGAGCTGGGCCAG
AGGGTTTGGGTTGATGGTTGCTCGCTTCTATTTGGCTGGATGCGAGGGACACGCCTGAGGGCGCGCTCCGGCATCAGGGCGACTAGAGGAGCGAGCTCCCTGA
ACGCGCGCCACGACACCCGAGGAGAACACC
>‘990809A-035.scf” came from CONTIG 33 at offset 0;
“C:\export\EG_DB\990820a\990820A-035.scf’ (13>36)
AGGCGGCCGCTCTATGATACTATT
>‘990809A-033.scf” came from CONTIG 34 at offset 0;
“C:\export\EG_DB\990820a\990820A-033.scf’ (61>588)
GCACGAGGCCCAAAGACACTGAAGTTTTCTGGAACAATGGCAGAAGTTGGGTTTGAGAGGAGGAGTGTTCTGTCTTAAAGCATGTGGACCAGAGGTCAGTAGA
TGATAGAAACATGTAAGTGTACATAGTAGTATTGTCAGATGTCAAAGATGCCAGGATGGAGGCTGGGTGGGGTCTAAAGTGGCATTAATGGGTTAATAAATTG
TCACCCCTATCCTCAGTTCTATGGTAGGTGAAATGTACAGTTAGTGTGGGGAGATGTTGTGTTTATTGGGTCTTTTTCTTTTACATAAAGATGAAGATCCACA
GGGTTGTATGGGTTGAGGGAGAGAGACAGAGAGAAGAGGTACAGAGCTGAAGGGTTGAGACAGGGAGGNAACTGACTCCTTTGGCTATAGATATAGGACGAAC
CCTATTGATTATTACCCAACATCAAGNNGATAGCAACAAAAGCGGCGGGGGGGCCGCCACAGGTGGGACCATGCTGGCTGGCATAAATTAACAGCCCCTCGGC
GAGGGACGGCCTN
>‘990809A-040.scf” came from CONTIG 35 at offset 0;
“C:\export\EG_DB\990820a\990820A-040.scf’ (60>587)
GCACGAGGGTGGGGTCCCCCTGGGGGTGGGGGGCGGGAGACATCTGGGCATCCAGCCCCCCCAGGTCCTCTTACTCTCTCTCTCCTTTCCTCCATCCACAGGT
GATAGTGAGGTGCGGAAGCTGGAGGTGGCGCTGGGCGTACATCTTCGAGATGCCAGGCGTGGGCAGAGGCTCCGCTCAGGGGCGCACGGGGTGGTTGCGGGAC
CTCCCAACGCCGGCAAAAGCAGCCTGGTGAACCTGCTCAGGGGTGGGGCGGGGGCGGGGCTAGGGGCAGGGGCGGGGCTGGAGCTAAGCTGCTGGGCTTGTGG
GGTAGGGAGGGGCCTGGGAGGGTGAAAACTGGCCGGGCCGGGGGGGGCCTAGGGAGGGACCCTCCATTCCACCACCGCTCCTCTGGCCACCCCACCCCGGCCG
GAGCTGGTCATCGGGCCCGAGCGGGACACCGGAGTCTGGACCCGGGACTGGCGATTCACGCTGTGGGACATGGGGTGGGAGGGGGGCTGGGCGGGGGGCGGGC
CAAAGAGGAGCGA
>‘990809A-036.scf” came from CONTIG 36 at offset 0;
“C:\export\EG_DB\990820a\990820A-036.scf’ (55>584)
CTCCCCCCGAGCGCCGCTCTGGCCGCACTGCGCTCGCCCTGAGCTCCGGGCTGCTGCTAAGCCAGCGGCGCTGTCGCCTCCCTCCAGTCGCCATCATGATCAT
CTACCGGGACCTCATTAGCCATGACGAGATGTTCTCCGACATCTACAAGATCCGGGGGGGGGCGGACGGGCTGTGTCTGGAGGTGGAGGGGAAGATGGGCAGA
AGGACAGAGGGGAACATCGATGACTCGCTCAATTGGTGGAAATGCCTCCGCTGAAGGCCCCGGGGCGAAGGTGCCGAAAGCACAGAATCACTGGGTGCNGTGT
TGTCTTGAACCATCACTTTGCAGGAAACCAGCTTACAAAAGAGCCTACACGAGTACTAAAAGATACATGAAGGAATCAATGGAAACTGTACACACAGACAGAA
GAGAAAACCTTTTGACGGGGCTGAGACAAATCAGCACATCTGCTATTTAAAATATATTTTTTTGTGAAACATGATCAATGCGGGTGGTTGTGGCTACGGAGGT
GGGNANCCATTTGATTT
>‘990809A-038.scf” came from CONTIG 37 at offset 0;
“C:\export\EG_DB\990820a\990820A-038.scf’ (54>610)
CAGCAACCGGCCTGCCTTCATGCCCTCCGAGGGCAAGATGGTGTCGGACATCAACAACGGCTGGCAGCACCTGGAGCAGGCCGAGAAGGGCTACGAGGAGTGG
CTGCTGAACGAGATCCGCCGGCTGGAGCGGCTCGACCACCTGGCAGAGAAGTTCCGGCAGAAGGCCTCCATCCACGAGGCCTGGACCGATGGGAAGGAGGCCA
TGCTGAAGCACCGGGACTATGAGACGGCCACCCTGTCGGACATCAAGGCCCTCATCCGCAAGCACGAAGCCTTCGAGAGCGACCTGGCCGCCCACCAGGACCG
CGTGGAGCAGATTGCCGCCATCGCCCCAGAGCTCAACGAGCTGGATTACTACGACTCCCACACGTCAACACGCGCTGCCAGAAGATCTGTGACCAGTGGGACG
CCCTGGCTCTCTTCCCACAGNCGCAGGGAGCCCCTGAGANAACGAGAGCAGCTGAGACATCGACAGCTGCACTGGAGTCGCCAGCGGGCGCCCCTCACACTGG
ATGANGNGCATGGAGACTCAGACAGTCATCGCCCACATCAGN
>‘990809A-042.scf” came from CONTIG 38 at offset 0;
“C:\export\EG_DB\990820a\990820A-042.scf’ (54>549)
CAAAATTCTGAAAGCTGAATTTGTTACATAGTCTCAGTGAGCTCTTAACAGAATAGTGTATGTTATTTGGGGGGAAAAGCAAACCTGAAAGGATTTTTCATGA
ATACTTCTTAAGCTTAAATTATTTATTTGTCTATGTCCAGGCTTAGTTGTAGCATGCGGGATCATTCATTGGTTGGTGTGCGTGGGCTTCTGTCTAGTTGTGG
CATGTGGGTTCAATAATTGTGGTGCACAGGCTTAGTTACCCAGGAGTTGTGGATCTTAGTTTCCTGATCAGGGATTTGAACCTGCGTGCCCTGCATTTGAAGG
TGGATTTCTCGACTGCTGGACCAGCAGGGAGTCCCTACTGAAATATTTTGTATTAAATAAAAGGGTTGGCTGGGTTCCCTCTGCAGGGCCCAGGCATCAATAC
AAGACAGCGCGGGGNGGGGCTGGGGCGGCGGGGGGGCGGAGTGAGGACAGTTAGCTGGAGGAGTTTGGATGGGGGGGGGGCGCGGA
>‘990809A-039.scf” came from CONTIG 39 at offset 0;
“C:\export\EG_DB\990820a\990820A-039.scf’ (53>591)
TTCTCCTCGGGCATCACGGGCTGCATCAAGAACCTGGTGCTGCACTCCGCCCGGCCCGGCGGCGCGCCCCCGCAGCCAGTAGACATGCAGCACGGTGCCCAGG
CAGGGGCCAACACACGCCCCTGCCCCTCGTAGGCCCTGCCTGCCCCGCACGGACTCCTGGGCCGCACCCCAGCCCCGCAACGGCGACTATATTATTATTAAAT
ATTATTATGATGATGATGATGAATATTTTGTAAGAAACCGAGGCGATGCCACGCTTTGCTGCTACTGCCCTGGGCTGGACTGGAGGGTGGGCACGTCACGCCC
TCCCGCCCCCACCCACAAACACACCTGGGCAGAGCCACAGGCTGTGGGCACAGCAGGTTGCACCAGAGCCGTGCCTCGGGGGCCACCAGACACGGGTTAGGCG
CAGTGGCTCATGGGTCAGACCGCCCCACACAGACCCCCCAGCAGGCTGCCGNCGTTGTCAGCTGGGCGGGCCCTATTCTGGAGCGCATGCTCACCGCCCTGCA
GCACTGAACCACAAACCGGAGAGGA
>‘990809A-045.scf” came from CONTIG 40 at offset 0;
“C:\export\EG_DB\990820a\990820A-045.scf’ (61>588)
GCACGAGGTGGAGAAGGGGGCAGACCTCAAGTGGGGGAGCCACCTGGGCTGAGGTGCCTGGGCCAAGTTAGACCTTGGGCCTGAAGGCTTCTGGTGGGTAGCC
GGCCCCCTCCCCACTGAAGCACGGAGCTCTAAGAAGTCAAACACGTGTTGACTCATTTGTTGGAGAAATTCAGCTCATGGGCTTCCTGCATTCCAGGGTGCTG
GTATGCCAGAAATTCTCTGGAGGAGCAAGCAGGGAAGTCTTGTCTTAAAGTAAAAATGCTAATAAAACATCTCTAGAATCTGCTCCTTCCTTTTCACCCAGAC
AGCCACTGACTAAATTACCTTTTGTTCTCATTTGCTGAATTGCCTTGGCTGCTTAATTGGCTTCNCTGTTTTTGGTTTCTNCACACCCACTTCATCTGTCCTC
CAATGGGTTCAGATGATATTAAAATGCANACTGCCACGTTCTGCCTTGGTTNCCCCACGCTGCAGATGAGGCAGATGCTAGTGGGCCTCTGGACCTCAGAACT
TGGGCTNATGAGG
>‘990809A-053.scf” came from CONTIG 41 at offset 0;
“C:\export\EG_DB\990820a\990820A-053.scf’ (55>485)
CAAAATTGACCTTAAACAAGGAAAGTTTGAAGTCACCATCTTTGACTTGGGAGGTGGAAAAAGAATTCGAGGAATCTGGAAGAATTACTATGCTGAGTCCTAT
GGGGTAATATTTGTTGTGGATTCAAGTGATGAAGAAAGAATGGAGGAAACAAAAGAGACAATGTCAGAAGTGCTGTGACACCCTCGGATATCCGGAAAGCCTA
TATTGGTGTTGGCAAATAAACAGGATAAGGGAGGGGGCTCTAGGAGAAGCTGATGTGATTGAGTGGTGTTTTTGGAAAGCTCGACATGAGCACACGTGCTGTG
TTAGATAAACCCTGTGTGCAGACTGGGATATGGAAAGAAAATGACATGTTCATTAAAAGGGCTTTTTTGGTTCTACTTTATTGCAGGGGCTTGTTGCCTTAAG
AACGCTCCAAAAACACA
>‘990809A-046.scf” came from CONTIG 42 at offset 0;
“C:\export\EG_DB\990820a\990820A-046.scf’ (53>593)
TGAGACATTCCCATGTTTCGGAGGATTTACAGACAGGAGGATTCGCTCACCCTCACATCTAGCAGGTTTTTGTAAACGTGACCCTTGGCTGCATCTCCCATCT
TCAGCACAGCTCAAGCACCCCAAACGTGTCCTTTCTCCCCCATAGACTGACAGGTGGGATCAGCTCCCCGGTAACCTTCTCTCCCTTCTCCATCTTCTCCCAC
ACCTTGTCCATCCATAAAAAGCAGATTTTGGGGGTCCTTCCACGCCTTGTCCCTTTCTTTGTGTCTTTTTTTTAAGTGATATTTTTAGAAATACATGTGAAAT
ACCAAGGATTAATGTCTGCCCCTCTGCGACCTCTCTTCACCTCTTTTTCATAAAGCTGCTCTTTATGTTGCTTACATGCCTPATATATGTTTGTGAAGATATA
TATTGAGAGTATTGTATATATTATATATTTTTGTTGGACATCGATCCTTCTGAACTCTGCCAGCCGNTTCTCTCTTCCTTCACATATCAGCAACGCGCCATAC
CCAGCCTNGAGCAANGGGGGAGAGNA
>‘990809A-044.scf” came from CONTIG 43 at offset 0;
“C:\export\EG_DB\990820a\990820A-046.scf’ (59>584)
GCACGAGGGGCATGTTGCGCGCCGTTGCGCTTGCCGCCGCCCGCCTCGGACCCCGCCAGGGCCGCCGCCTGCTGTCCGCCGCCACCCAGGCCGCGCCGACCCC
CAACCAGCAGCCTGAAGTCTTGTACAACCAGATCTTTATAAACAATGAGTGGCATGATGCCGACAGCAAGAAAACCTTCCCCACGGTCAATCCATCCACTGGG
GATGTCATCTGTCACGTGGCTGAAGGGGACAAGGCAGACGGGACAGAGCAGGGAAGGCTGCCCGGGCCGCATTCCAGCTGGCTCGCCCTGCGCCGCATGGACG
CGTGCGGGGGGGCCGGCTGTGAACCGCCTGGCTGTCTGATTGAGGAGACCGACCTACTTGCAGGCTGGAGACCCTGAAAGACAGCCCTTATATCTCTACCGTG
ATCTGACAGGCTCAGTGCTGCGTCTTGCGCTGGCTGCAAACAGGAAACATCCATGACGGACACTCAGTCCCGCAGACGGGAGGGGGCGAAATCAGGACTCCGC
CGGCGCTGTAC
>‘990809A-054.scf” came from CONTIG 44 at offset 0;
“C:\export\EG_DB\990820a\990820A-054.scf’ (62>492)
GCACGAGGCTCACTTTGGTTTTTTAAATGACGTTATTTCTGAGGCTTGACGTCCCACAAGCTAATCTGTTTCTTCAAGGCCCTGGACCGGCAAGGGCAATCTA
GGCTATGGGGAGTGTTAGCTTGTGTTGCTGACTTAAGACTTCAGCCCTTTCGCTGTCACCTGTACCAAGTGCCAGGCCAACAGAGGGGGGGGGGAGGCAGCTT
CACGACGGGGCTAGGGGAGTCTGGAAGGAAGAAGCTGCACGCGGGGAGGCTGGGCCTGGGGAAATGAGCATCTTGGACTCATAAGGCCTGTTCTTCTTTGTTC
CTGGTCTGGTCCACAGGCACCAATTTTTCTTTTTTTTTGTTTGCTTCTTTGGTTTTGCTTCTGGAGCCCATGTTGTGGCTGAGGCAGACCGCGGAACCCACAA
AGAGGGGTGGGCCCCTGCC
>‘990809A-051.scf” came from CONTIG 45 at offset 0;
“C:\export\EG_DB\990820a\990820A-051.scf’ (56>551)
CTCAGATCGCAGCGGAAGAGTCGTGCTTTTCTAACGTTTCTTTAGCTTCCAAATCCCGACATACAGAGGCTGGTAAAAGCAGAGCAGATCTGGTCAGGTCCTG
AGACCGCTGAGTCCAGAGCAATGTTGCTGAAGACAGTGCTCTTGCTGGCCTTGGCGTCCCAGGTGCTAGTCCTGGAGAACGGGCTCCTGCGGAAGCCACCCAT
GGGGTGGCTGGCCTGGGGAACGCTTCCGCTGCAACATCGACTGCAGTGAGGGACCCGAAGAACTGCATCAGTGAGCAGCTCTTATGGAGATGGCTGACCGGCT
GGCGCAGGATGGATGGGGGGACCTGGCTACGTATACCTTAACATCGTGACTGCTGATTGTGGGCGTGATGCCAAGGCACCCGGTGCGGGACGCAGCGCTTCCC
ACGCATGCCTTCTGCTGCTTGCTACTCCTGGCCGAGCTGGCTTTACAGACTGGNCACTCACTGCTGGGTACCGGCCCCGCTGACA
>‘990809A-034.scf” came from CONTIG 46 at offset 0;
“C:\export\EG_DB\990820a\990820A-034.scf’ (60>595)
GCACGAGGCACTGGCTGGGAAGCACGGGGGATGACCTTCGTCGCACGAAGACGGAGATTTCTGAGATGAACCGGAACATCAACCGTTTTGCAGGGCTGAGATC
GAGGGTGCTTAAAGGCCAGAGGGCTTGCCTGGAGGCTGCCATCGCTGACGCTGAGCAGCGTGGGGAGATGGCTGTTAAGGATGCTCAAGCCAAGCTGGCCGGG
CTGGAGGCCGCTCTGAGGAACGCCAAGCAGGACATGGCGCGGCAGCTGCGCGAGTACCAAGAGCTCATGAATGTCAAGCTGGCCCTGGACGGTGGAGATTGCC
ACCTACAGGAAGCTGCTGGGGGGCGAGGAGAGCCGGCTGGGTCTGGATGCAGAACATGTATATCCACACCAAGACACCGTGGCTTCGCAGTGCCTGACTTCGC
CCACGGACCCTGCTCACTACACCCGGCCCGCTCCTTACCCACCACTCCACCGGGGTGTGAAAGAGGGACCCGAGGGAGCGGGCCGGCCCTGTGTCCGCCAAGG
AGGCCCGGCGCCTCCCCCCCC
>‘990809A-052.scf” came from CONTIG 47 at offset 0;
“C:\export\EG_DB\990820a\990820A-052.scf’ (62>521)
GGACGAGGGCCAACCGGGGTCGGAGAAGATCTCAAGATGGCTGGACGGGAACTTGCTCTAAAAACCATTGGACTGGGGTAGCTTTTGGGGGGGATCATCCCTC
GGAACCAGAAGGCGGGCTAACTCCTTGAAGTCCTGGAATGAGACCCTAACCTCCAGGTTGGCTACTCTGCCTGAGAAGCCACCTGCCATCGACTGGGCTTTCT
ACAGGCCAACGTGGCAAAGGCTGCTTGGTGGATGACTTGAGAAGAAGTTTATGCCTCGAGGTGTCTTACCAGAGATAAATTACTGCCAGGTGGTGCTGAAGAA
AGAAGATGTGAAAGTGTGTGGGTTTGTCTCATCAAAACAGATTATGATTGATAGAGTGTGGAAAGAGGAATATTTCGTCAATGACTTGGGATGAGAAGATTCA
CACCAATAACAAGAGACCCCTGCTCCAGCATGGACTTTGTGGTGCG
>‘990809A-050.scf” came from CONTIG 48 at offset 0;
“C:\export\EG_DB\990820a\990820A-050.scf’ (60>423)
GCACGAGGGGGATATGTGCCCAGGTCCTGCCCTACCTGTCCCAAGAGCACCAGCAGCAGGTCTTGGGAGCCATTGAGAGGGGCTAAGCAGGTCACTGCTCCTG
AGCTGAACTCCATCATCCGACAGCAGCTCCAAGCCCACCAGCTGTCTCAGCTGCAGGCTCTGGCCCTGCCCCTGACCCCCCTGCCTGTGGGGCTGCAGCCCCC
TTCTCTGCCGGCGGTCAGCGCAGGTACCGGCCTCCTCTCGCTGTCGGCGCTGGGCTCCCAGGCCCACCTCTCCATGAAGACAAAAACGGGCATGATGGTGACA
CCCACCAGGAGGACGACGGCGAGAAGTCGATTATGGGCGGTGGTGGGGNGGGGGN
>‘990809A-015.scf” came from CONTIG 49 at offset 0;
“C:\export\EG_DB\990820a\990820A-015.scf’ (61>564)
GCACGAGGCTCCGGTGTCCCCGCGCCAGAGACGCAGCAGCGCTCCCTCTGCCCACACCCACCGCGCCCTCGCGCTCGCCTCTCCTTCCGGAGCCAGTCCGTGC
TACCGCAGTCGCCCAGTCCACCACCACCCTCTGCAGCCATGTCCACCAGGTCCGTGTCCTCGTCCTCCTACCGCAGGATGTTCGGGGGCCCCGGGACCGCAAG
TCGGCCGAGCTCCACCCGGAGCTACGTGACCACATCCACCGGCACCTACAGCCTGGGCAGCGCGCTGCGCCCCACCACCAGCCGCACCCTCTACACCTCGGGC
CCGGGTGGGCGTGTACGCCACGCGCTCCTCGGCCGTGCGCCTGCGGAGCGGCGGGGCCGGCGTGCGGGTGCTGCAGACTCGGTGGACTTCTGNTGGCCGACGC
CTCAACACCGGTTCAAGACACCGCACCACGAGAGTGGAGCTGCAGAGCCATGACGCTCGCCACTACATGACAGGCGCTTCTGGACAGAAACA
>‘990809A-096.scf” came from CONTIG 50 at offset 0;
“C:\export\EG_DB\990820a\990820A-096.scf’ (60>437)
TGGTACGGGGATAGCGTCTGCGCTATGGCATATACGTCATCAACCAGCACGGGGAGGACACCTTCAACCGGGCCAAGCTGCTCAACGTGGGTTTCCTAGAGGC
ACTCAAGGAGGACTCCACCTACAACTGCTTCATCTTCAGTGACGTGGACCTGGTCCCCATGGGATGACCGCAACCTGTACCGCTGNGGTGGCCAGCCCCGCCA
CTTTGCCATTGCCATGGGCAAATTTGGCTTCCGGCTGCCCTATGCTGGCTACTTCGAGGGGTGTCGGGCCTGAATAATCCCAGNTCCTGGGAATCATGGCTTG
CCAACGAGTCTGNGGTGGGTGGTGAGATGATGACTCTTCACCGGTCTCCTGCTTTGATGAAGATCTGGGT
>‘990809A-055.scf” came from CONTIG 51 at offset 0;
“C:\export\EG_DB\990820a\990820A-055.scf’ (60>587)
GCACGAGGGTGTAATCTTGTGATATCACCTCTGTAAGCCTGGATCTCCCCAGGTGTCAAAGGAGGCAGTTGAATAAGAGGAACTATGAACTCTTCTATCTGTG
TTATATAGACGGCCCATTTCAATCTAGAGCAGGGGAATGCCATCCCAAGAGGGCACTTTTACAGAGGAGGGGGGTTTGTCAACATCTTTGGTTGACATTACTC
TGGTGTAGGGAGGGACGGAGAAGGCAATGGCACCCCACTCCGGCACTCTTGCCTGGAAAATCCCATGGATGGAGGAGCCTGTAGGCTGCAGNCCATGGGGGGT
CGAGAGTCAGACACGACTGAGCGACTCACTTTCATTTTCACTTCCTGCATTAAGAAGAAAGGCACCCACTCCAGGTCTGCCGGGAATCCCAGATGGGGAGCCG
TGCTGTGCTTGGGGACACAAGCGACACACGAGGACTAGCGCGCGCAGGGGGAGCCGNGTTGATAGATGCTCAACCTCACGGGACCTGCGGTGTACATAGATAT
ACTATATTTGCGA

| | |
|---|---|
| >‘990809A-064.scf” came from CONTIG 52 at offset 0; | “C:\export\EG_DB\990820a\990820A-064.scf’ (59>611) |
| GCACGAGGAATTTATCAAAAATCCCAATAACTCAACACAGAATTTGCACCCTAACCAAATATTTACAAA | |
| CACCACTAGCTAACATAACACGCCCATACACAGACCACAGAATGAATTACCTACGCAAGGGGTAATGT | |
| ACATAACATTAATGTAATAAAGACATAATATGTATATAGTACATTAAATTATATGCCCCATGCATATA | |
| AGCAAGTACATGACCTCTATAGCAGTACATAATACATATAATTATTGACTGTACATAGTACATTATGTC | |
| AAATTCATTCTTGATAGTATATCTATTATATATTCCTACCATTAGATCACGAGCTTAATTACCATGCCG | |
| CGTGAAACCAGCAACCGGCTAGCAGGGATCCCTCTGTCGCTCGGGCCCTAAACCNGGGGGTCGCTTCT | |
| ATGAATTNTCCAGGCTCTGTTCTTTCTCAGGCCATGTATTAAACGTGCTTCTTNGTCTAATAGAATCTGA | |
| TGACTATGCTATAGCCAGCTACCATACGGCTGCNTCTTGTTTTTTGTGAGCTGACTAGTTGCCGN | |
| AAN | |
| >‘990809A-094.scf” came from CONTIG 53 at offset 0; | |
| “C:\export\EG_DB\990820a\990820A-094.scf’ (54>456) | |
| CGCACCGTCAGGCTGTACTGCAGGGCCGCGGGGGTGCCCAGTGCCACCATCACCTGGAGGAAGGAAGGGGGCAGCCTCCCCCCACAGGCCCGTGCAGAGCGCA | |
| CAGACATTGCCACCGTGGTCATCGCCGCCATCACGGCCGGCGACGCCGGCTTTTACCTCTGTGTGGCCACCAGCCCTGCGGGCACCGCCCAGGCCCGGATTCA | |
| AGTGGTCGTCCTTCCAGGTGCCACCACCCCACCGGTCAGGATTGAGTCCTCCTCGCCTTTTGTGACCGAAGGACAGACCCTGNACCTCAACTGCGGGGTGTCA | |
| GGGCTGGCCCACAGCCAGATCACGTGGTGCAGCGAGGGGGCAGCCTGCCTCCCACGCCCAGTGCGCGGCTCCCGCTGCGGCGCCCCAGTATTAC | |
| >‘990809A-058.scf” came from CONTIG 54 at offset 0; | |
| “C:\export\EG_DB\990820a\990820A-058.scf’ (56>578) | |
| CTTCGTCCCTCTGGGGTGGAAACCGGAAAATTCTCAGGATAGCGTGTACCTCCAGTACTGTAAAGTCTGCCAAGCATACAAGGCACCACGGCCACATCACTGC | |
| AGAAAGTGTAACAGATGTGTGATGAAGATGGACCATCACTGCCCTTGGATCAACAACTGCTGGGGCTATCAGAATCATGCTTCCTTCACGCTGTTCCTCCTTT | |
| TAGCACCACTGGGCTGCATTCACGCTGCCTTTATTTTTGTTATGACCATGTATACGCAGCTTTATAATCGGCTCTCCTTTGGTGGAACACGGNCAAGATTGAT | |
| ATGAGTGCAGGCCGCAGAGACCCTCTCCGTTATTCTTTTGATTAGCTGCTTTGCGCCACCTGTTTGCCTGGGTTTAGCTTAGAACACCTACGTCGGTGTGNTT | |
| TTATCAGAGAAATATCTAAAACAACTCATGATATGATGAGAGAGTAGATGATTATATATATAATGATTTGTTTCTTGTTGGAGGATGAACTCACAGATAATGC | |
| GGTCGAGGN | |
| >‘990809A-059.scf” came from CONTIG 55 at offset 0; | |
| “C:\export\EG_DB\990820a\990820A-059.scf’ (56>604) | |
| CTTTCTGAAGAGCCAGGAATTCCTTCAGGCTCGCACCCCGACCTCAGCCAGCACCCCCATCCCACCCACCCCTCAGGCTCCCTGGCCTGCTGTAGATGCCGAG | |
| ATCAGAGCCCAGGATGCCCCTGTGTCTCTGCCCCAAGCACGAGTTGGGAGGCAACAGGTGCCAGAAGTCATGTGGGCTGAAGCCAAGGTGGCCATCCCCGCCA | |
| GCGTCCTGCCAGGACCAGAGGAGCCTGGGGGCCAGCAACAAGAGCCCAGACCAAGCCAGACCCCTGAAGATCATGCCTCCCTGCTCCAGCCCTCACCCTGACC | |
| ACTCCAGTCTAGAGACCAAAGATGGAGAACCCAGGCATCTAGAGAGACCAGCAGATCCCAGGAGGAGATGAAGCCACTGTGGGGCTGACAGAAAGAAACAAGG | |
| TAGAGCCAAGCAGGAGCAGCTGCAGAGAAACGGCAGAGAGCGTCTGGACGGAAAATTCAGATGCCAGCCTTGAAAAGAGCTTGACCTTGGATAAAATCAAGCA | |
| TGNTCCTGGAACAACCTGAACTTAACCTAAAGGG | |
| >‘990809A-084.scf” came from CONTIG 56 at offset 0; | |
| “C:\export\EG_DB\990820a\990820A-084.scf’ (56>504) | |
| CTTCGGCACGAGGCCCCCTTCCTAGGTCGGGGGGGACTTTTGTCTACCCTTCCCTCACCCTCGAGGACCCTAGTGGCCTCTGATGCCAGGGGTGCAGTGCCTG | |
| CCCAGTGAAGGAAAGTAGAAGAAAGAGGCAAGGCCCGCTCCCGGCTCAATGTTTGACCTTCCCAGGCCCCATTCGCCCCTTTATGTAACTGTCTTCTTATATA | |
| AATGGTGATCTTTTTCTCTTCATCCACCATTGATGTTGGGTCAAGAAACTGGGCTGGATGGGATGAGCACCCATGGTCCGTCCTGTGCATCTTTTCCTTGCTT | |
| ATTACGTGGTTTGGGCTGTCAGCCAGATTATATTCCCCAACCCATTTTTGCCTTCCTCCTTCCCGGGTGCCTTGGTGTTTGGTTTATTCCGGGGTCCAGTPCG | |
| TATTTTTGGGGGTTCTTGGTGGGGGTCTTTCTTCTCA | |
| >‘990809A-063.scf” came from CONTIG 57 at offset 0; | |
| “C:\export\EG_DB\990820a\990820A-063.scf’ (62>547) | |
| GCACGAGGGTGGGTTAAGCATGAATCCTTTTACACAGTCATTAATATTGTCTTTTAGGGTTTATGTAGTATTCTATAGTTTTTGGAATGAACATAAGAAAAAT | |
| AAGTAGTACTTCATAGATCCTGCTGCATGCCAGACCCTATTCCAAGTATTTTACATATATTGATTTATTTAATTATCAGAATGATCTTATGTAGGAACTACTG | |
| CTTCTATATCAATCCAGCAGTCCCTGATTTCCACACTGTAGATGAGGGAGGATAGCTCACAGGCAGCAAATGAAGCAACAATGTGCTTCACAGCANACTGTTA | |
| GCAGATTCTTAAGGCAAAAAAAATGGCGATGGTTGTCTCATTATCAGGAACCTAAGCACGCTGATGTCCCCGCGTCCCCTCTGTCCACTGGGGAGACGCCACA | |
| CCCTTCTTATGTTTCCCTTGAGCATAACATCTACCTTAACACCCCCCCGCATTATCAGCAAAAGAA | |
| ACACAA>‘990809A-060.scf” came from CONTIG 58 at offset 0; | |
| “C:\export\EG_DB\990820a\990820A-060.scf’ (58>515) | |
| GGGCACGATGCTCACTTTGATTTTTTATATGACGTTCTTTTCTGATGTTTAACACCCCACAAAATATTTTCATGATTTACGTCCCTGTGCACCCAAGGGCCGT | |
| CGATGCTATGAAGAGCGATGGGTTGCTGGGGGGCCTTAAAACTGAAAAGCTTTCGGTTGCCCCTGTGCCAATCACCAGGACAAAAGAGGTGGGGGGGAAGCAG | |
| CTAGACAACTACGTCTAGGGGAGCGAGCTGGAACAAGGGCACACACGGACGCCGCGCCTGCCTGAATGAGGGACAGGGACTCCGACGACCTGTGTTCTTGTGA | |
| TTCTGGACGTGACACAGGCACTCTGCGCTGCGCCTTTCTGTACGCTCTCTTGCTTCGCTGCGGGCGGCCCTTGAGGCGGACTGAGGATGAGCGCGCAACGCCA | |
| CACGAGGCCGCGGGCCCCTGCCCAGCGCGGCGCGAGAGGAAGTGCCC | |
| >‘990809A-091.scf” came from CONTIG 59 at offset 0; | |
| “C:\export\EG_DB\990820a\990820A-091.scf’ (56>557) | |
| CGGACTGGGAGGTGAACCTCACCGACTCCTTCTGGAACTGGGAGAAGGGCTTGGTTCTTGAAACTCCTCAGGTCGGACTTTTTTTTTTTTTTTAAACTGGGGG | |
| CTATGCTGCCCTTTCAATAAGGTTTTTCAATCGTTGGTGTTTGCGTTTCCAACTTAAGAGAATTCCAGGCACTCCCCTTCCCCCTCCAGTGACATACTTGGGC | |
| AAGCGGTCATCGTFFGCGTCATGGGGCAGACGGGGGGAGCTTCCTGCTGCCGNGCGGGGGTGGGGGCCGGGAGGAGGACCTGGGTGTGGGCCGCCCTGGGGAA | |
| TGGAGGNGGGCGGCCTGAGCACTGCGCCTGCTGCGGTTATTGCCCGAGCCCCTCGCCTCGGGGTAGAGGNCCGACTATTTCTTTAAAATTTTTTCTGTGGGCG | |
| TTGAGTGGGATGTCACGTCCAGCTGCCTCAACCCACAACACACCGACGTCTGCCGATACAAATGAGGAGCAGAGCGACTGATCGGCTGCGA | |
| >‘990809A-088.scf” came from CONTIG 60 at offset 0; | |
| “C:\export\EG_DB\990820a\990820A-088.scf’ (56>586) | |
| CTTGATTTGAGTCTGTTTCTAATCCCTGTGTCCTTTGCTCTCCAGGGGATGTGTTCCTTTCATATTTGTTGGCACCCGAGAAAACATCAGCAATGCTCAGGCT | |
| CTGCTGGAATATCACCTCTCCTACCTGCAGGAGGTGGAGCAGCTGCGCTTGGAGAGGCTGCAGATTGATGAGCAGCTTCGGCAGATTGGGCTGGGCTTTCGGC | |
| CTCCTGGAAGGGGGCGGGGCAGCGGCAGCAGGGACAAGGCTGGATATACCACTGATGAGAGCTCCTCCTCTTCCCTTCATACCACACGAACCTATGGGGGCAG | |
| GTATGGGGGCCGGGCCGGGGCCGGAGGACAGGCGGTCCTGCCTATGCTGTGAGACGNATCAGAGAAGAGGGAGAGCCCCCCCGGCTGGCCCGGCGACGGGATC | |
| CCCGCCCGGGGAGAAAGCCGAGCGNCTATAGAGGCGGGTAGGGACCCCACTGCCCCGNCCACTAGATCACTCTTATGATATGTAGCTGAGACAACGAATCTTC | |
| AACACGGCACATCACA | |
| >‘990809A-078.scf” came from CONTIG 61 at offset 0; | |
| “C:\export\EG_DB\990820a\990820A-078.scf’ (55>588) | |
| GTTTTTTTTTTTTTTTTTTGATGGAATGTAAATCTTTTATTTAAACAGTTGTCTTTCCACAGTAGTAAAGCTTTGGCACATACAGTATAAAAAATAATCACCA | |
| ACCATAATTAGACCAGATTCCTCTTATCAACTGCATACTAAGTATCTTCAGTACAATTTTTTTTCCATATAAAAATACTGGGAAAAATTGATAAATAACAGGT | |
| AAGAAAAAGATTTCTAGGCAATTTACTAGAATCATTGGGAAAAGTGAGTACTGGGGCTGTTCTGAATACCACAGTACAAAGGACATGCTGTTCCTACAATATT | |
| GCGGGCCAGTCAGTTAAGTGGAAGCAGAAGTGTTCAGGTAACTTTCCTACTTAAAATTTGGTAATATCATTTCAAGACATTTGTATCTTGGTTGGGTGCATGT | |
| GCTCCCTAGGATCCCATCCAAATCACAGTAGATCACTCA7FITAAATCTGATGCATGGATATTrGAGAATGATACCTCTGCTCATGATGAGAAAGCTGAACAC | |
| TCAGGGAGCTGGAGAGCGT | |
| >‘990809A-032.scf” came from CONTIG 62 at offset 0; | |
| “C:\export\EG_DB\990820a\990820A-032.scf’ (53>590) | |
| TGGTTTTCTTAGGCACGGGGGGAGCTGAGTAGGTGTGGGGATGGGACAGGGAAGGGCAAAGGACAGAGCGGGGGGACCTTTGCCTCTCCAGGTGCCCCACGGC | |
| CAGCCCCCCGCGTCCTTCCTGCACTGCTCCCACACCCCACACCCCCAGGGCCCTGAGGGAAAGACAGGCCCCGAGGCCCCAGGCTGGAGAGAATAGGCCGAGG | |
| CATGATGCCGCACTCCTGGCCCCGAGACTTCCCCTTCATCCCCTCCCACTGCCCACAGACCCCTTTTGACTCTCATCCTGAAGCCTAGAAAGAGAGAGAAGCG | |
| GGGNGGGGTGGGTCTGTGGGGNGACGGGCGGAGGAGGCGGGGAGCAGGGAAAGGCGGGAGCCCTCTGTGCTGGTTTTTACCAGATACACAGCAGCTTCCAATA | |
| TATTATTCACCCCTGAAAAAAAAAAAAAATGAGGGGGCCGGGACCATCGNCTATGGAGNGATACATCATGNCGCGTTTAGAGGGATGGAAACTGCGTACACTA | |
| TGCTGACCATCCGTTGCGGGGGT | |
| >‘990809A-090.scf” came from CONTIG 63 at offset 0; | |
| “C:\export\EG_DB\990820a\990820A-090.scf’ (60>584) | |
| GCACGAGGCTCGGGAGGTCAGAAAGCCGGGCCGCGGGCGGCACCGAGAACTGGAGCTGGGATCGGGGACGCACAGAGGTCAGGGGAAGTAATCCTGGACCATG | |
| ACTCAGCAGCCACTTCGAGGTGTGACCAGTCTGCGTTTCAACCAAGACCAGAGCTGCTTTTGCTGTGCTATGGAGACAGGTGTGCGCATCTACAACGTGGAGC | |
| CATTGATGGAGAAGGGGCATCTGGACCATGAGCAGGGGGGCAGCATGGGCCTGGTGGAAATGCTGCACCGCTCCAACCTGCTGGCCCTGTGGGCGGGGGTAGC | |
| AGCCCCAAGTTCTCAGAGATCTCAGGGCTGTCTGGGACGATGCCCGGGGGGGCAGGACTCCAAGACAGCTGTGCTGGAGTTCACTTCACAAGCCGGCGGCTGG | |
| CGCTGCGCATGACAATCGGATCGGCTGAGAACGCTTATGGTTCTCTCCTGACATCCGAAGCTGTGATTGCACCGGACACCCAGGGCTTGGACTTGTCCACTGA | |
| AACAGTGTGG | |
| >‘990809A-087.scf” came from CONTIG 64 at offset 0; | |
| “C:\export\EG_DB\990820a\990820A-087.scf’ (61>433) | |
| GCACGAGGCGAACGAGCAGTACCGGGCGCTGCGCCCCGACCTGGCGGTAGGGGGAGGGTGGATAGGAGGGGTGCGGCGGCGGGGGGGAGCGGGGGAACCCGGA | |
| CCCCACAGGACATGCGAGGACGGGGGGCCTCTCCCCTGGGTCAGTACTGTCTTTGCTCCATGTCCAGACATCTTCCTGTTTTAAAATGAACAAAGCTCTCACT | |
| TCAAAATCCACACTTCATTTGGGACTAGACAGTCGGGGGTTGGGGTTGTTTCCTTACTGCTAAACAAAATCCTCGACCCGGGACTCTGATTTGGGGACCAAAA | |
| GCCCCCCAGAACCTCACCTGTGAGCCTCGCTGTGTGGTGCGGGGAGGGTGGGTTGGGGGNNNGGG | |
| >‘990809A-067.scf” came from CONTIG 65 at offset 0; | |
| “C:\export\EG_DB\990820a\990820A-087.scf’ (59>555) | |
| GCACGAGGCTGCAGATTCCYFCCTATAACTATTTATAGGTAGTCCTGACTCCACTACCCGCTTCCGAAATGGGCTGTTCACGCGGAAGAGGCTCACGTTTCAC | |
| ACAGCAGAACCCGAGAGAATGGTGTGAACTGTCGGGGGGGAGTGCATGCAGGAGGCAGACTGAGTGCCCTGCTGTGCTCACCTGTGCTTTATCACTCTTCTAG | |
| CATGGCGTCCACGCTGGTCTCCCTCGGGAACCAGCTGTGTCAGAGGCCCCTCTGTGGCTGGATGTCTAGCTTGCTCCGCCAGGCACAGCGGNGCTTTTGGGGG | |
| CCGCAGGGGGCGGCGCTGTGCTGGGCTCACCCCGCGCGGNNACACGCTCCCTCTTGGCCNCACGGGTGTCCGGTCTCCCTCTCTGGCTTCGCTGTCCGCATTA | |
| GTCACANGAGAGCCTGGCTCCCCTGCCCTTATTTGNATGAAATGGCTAGATCTGCCTTACTCTTACTGATGATGAATTGATATGC | |
| >‘990809A-068.scf” came from CONTIG 66 at offset 0; | |
| “C:\export\EG_DB\990820a\990820A-068.scf’ (56>612) | |
| GTTGAGTCTAGGAACCGTCCCAGCATGGCTCCCCTCCACCGCCACCACCACCACCATGTCCCACCCCCTGCGATGGCAGGTGATCTAGCTCAGGGGGGCCTCC | |
| CAGGCTGAGCAGGAAAGGAAGTTTCCAGAAAACCTGGGCTGGGGGAGGAGTCCTGGGGACAGCAGATGCCTGCCGCAGAGGGCTGGCTGCCTGTGGACCCTTC | |
| CCAGCTCAGTGGAGGCCACGTTAGGGGCCCTCAAGCAACCCAGAAGCACAAATTGGTGGTTTGGGGCCACGCCCAGCTGGGCTGGCATCCACGAACCTGGAGA | |
| GTTGGCTATGGCAGCACCAGGGCCTCGGCCCCACTCCCCTCCCAGGGTCCCCGTCCTTTCCCCCGCCAGGCTCTGCTCAGGCCAGCCCCTGAGCCGNCAGCGG | |
| GCCCCTCTACCGGGGCCTGGTCTTGAGCACACCCTGTCCACTCACCCACTCTNNTTCCTCGCCACCCTTCCCCGCACCATCGCCCTATATCGGGTTGCTCTCA | |
| GGCCCACTGAGTCACTTTCTANNTTGTCTGGCCTGCGCGCGT | |
| >‘990809A-069.scf” came from CONTIG 67 at offset 0; | |
| “C:\export\EG_DB\990820a\990820A-069.scf’ (60>613) | |
| GCACGAGGGGAAGCCCATGTTTTATGTGTGCACACACACACAAATGTACACACACTCATGGTCTGCCAGTCTAGCAGTGGGAAAATGAAGATGAGGCAGGGTC | |
| TGAATGTCCACTTGCTCTCACTGACTGCCACGGAGCATGGTATGTGAAGTGGGGTCTTATCTGGAGAACTGTCCATCAGGGCTGTAGTCCTGGCTACAGTCCA | |
| CCAGGGCTGTAACATGCATTATCCATTTACAGTCAGCGAAACCATCGATAGAAAAGCCACAACAACAAGGGAGCTGCAGGAAAAGAATCATGTAAGCCCTCCT | |
| GGGTCCTTCATGGCTTTAGGAAATAGGGAAAGGCACCTAAAATGAGCAGGAAGGGCAACTAGAGTGGGGTTGGAGGTGGGGCAACCACATTTCAAGGGCTGCC | |
| AGTCTCAACGAAGCTGCCCAAAGCGACACCTATGGTGTGGTGTGTGCCCGAAGGGCCAGCAAGAGGAGTCTACATTTAAAGCTTACGTGACTCTTGTGCCATG | |
| CTAAACCACCTCAAGCAGGGGGGCCAACCCGGCGGGCGN | |
| >‘990809A-075.scf” came from CONTIG 68 at offset 0; | |
| “C:\export\EG_DB\990820a\990820A-075.scf’ (60>563) | |
| GCACGAGGGTGAAATTCAGAAGAAGGCGAGAGGGCAAAACTGACTACTATGCTCGGAAACGATTGGTAATCCAAGATAAAAATAAGTACAACACACCTAAATA | |
| CAGAATGATTGTTCGTGTAACGAACAGAGATATCATTTGTCAGATTGGTTATGGCCGTATAGAAGGAGATATGATAGTTTGTGCAGCTTATGCTCACGAACTC | |
| CCAAAATATGGTGTGAAGGTTGGCCTGACAAATTATGCTGCGGCATATTGTGCTGGCCTGCTGCTGGCCCGCAGGCTTCTTAATAGGTTTGGTATGGACAAAA | |
| TTTATGAAGGGCAGTCGAGGTGACTGGAGATGAATACATGGGGAAGCTCGATGTCACCTGTGCCTCACCTGTACCGGTGCGGACTGCCGCACACTACGGGATA | |
| AGTTTTGGGCCTAAGGAGCGCGNGGAGCTGCTTCCTCCAGACCACGGTCTGNTTGATTAGAGCAAGATCAGGCTGGACACGAGCCAATGCCA | |
| >‘990809A-074.scf” came from CONTIG 69 at offset 0; | |
| “C:\export\EG_DB\990820a\990820A-074.scf’ (62>380) | |
| GCACGAGGAAACAGGTTATTTTACCCTACTGATGATGTGTTGTTGCCATGGTAATCCTGCTCAGTACGAGAGGAACCGCAGTTCAGACATTTGGTGTATGTGC | |
| TTGGCTGAGGAGCCAATGGGGCGAAGCTACCATCTGTGGGATTATGACTGAACGCCTCTAAGTCAGAATCCCGCCCAGGCGGAACGATACGGCAGCGCCGCGG | |
| GAGCCTCGGTTGGCCTCGGATAGCCGGCCCCCCGCCGCCCCCGCCGGCGGGCCGTCGCCCGCGTCCCCCGGGGCGCGGCGCGGGGCGCCCCCGCTGCGCGTCG | |
| GGACCGGGG | |
| >‘990809A-086.scf” came from CONTIG 70 at offset 0; | |
| “C:\export\EG_DB\990820a\990820A-086.scf’ (62>593) | |
| GCACGAGGCTAAGACCCGTGTGCAGCAGCGGCGGGCGGGGGTAGAGGCGGGGGCGGGGGCGGCGGCAGCGGCAGCGGCAGCGGGGCTCGGGAGGCAGCGGTTG | |
| GGGTCGCGGCGAGCGGACGGGGTCGAGTCAGTGCGTTCGCGCGAGTTGGAATCGTAGCCTCTTAAAATGGCAGATGATTTGGACTTCGAGACAGGAGATGCAG | |
| GGGCCTCAGCCACCTTCCCGATGCAGTGCTCAGCATTACGTAAGAATGGCTTCGTGGTGCTCAAAGGCCGGCCATGTAAGAATGTGGAGATGTCAACTTCTAA | |
| GACCGGCAGCACGGGCATGCCAAGTCCATCTGGTTGTATGACATTTCATGGAAGAAATACCAGATTCTGCCATCACTCATATAGGAGTCCCCACATAAAGAAC | |
| GATTCACTGTTGCATCAGATGATACTTCACTCCCAAGAAGGGAGTGCGGAGACTCCGCGCCGAGAGACCTGCAGAAAGACAAAACACGGGAGAAAATTGTCAG | |
| GCGCCCCTGACAGGCG | |
| >‘990809A-072.scf” came from CONTIG 71 at offset 0; | |
| “C:\export\EG_DB\990820a\990820A-072.scf’ (293>632) | |
| CAAATTGTAAGCGTTAATATTTTGTTTAAAATTCGCGTTAAATTTTTGCTAAATCAGCTCATTTTTTAACCAATAGGCCGAAATCGGCAAAATCCCTTATAAA | |
| TCAAAAGAATAGACCGTGATAGGGTTGAGTGTTGTTCCAGTTTGCAACAAGAGTCCACTATTAAAGAACGTGGACTCCAACGTCAAAGGCGAAAAACCGCTTC | |
| AGGCGATGCCCCATACGGAACCATACCCTATAAGTTTTTGTGTCGAGTGCCGTAGCACTAATCGAACCTAAGAAGCCCGTTTAAGCTTACGGGAAGCGGGAAG | |
| TGGAGTAAGAGGAAAAGGAAGGGGGGCCCTGC | |
| >‘990809A-073.scf” came from CONTIG 72 at offset 0; | |
| “C:\export\EG_DB\990820a\990820A-073.scf’ (60>570) | |
| GCACGAGGAGAAGGGGGACGTGGTGCTGCAGAGTGACCACGTGATCGAGACCCTGACCAAGACAGCCCTCAGCGCTGACCGAGTGAACAACATCAACATCAAC | |
| CAGGGCAGCATCACGTTCGCAGGGGGGCCCGGCAGGGATGGCATCATTGACTTCACACCCGGCTCGGAGCTGCTCATCACCAAGGCCAAGAACGGGCACCTGG | |
| CTGTGGTGGCCCCGCGGCTGAACTCGCGGGGATGAAGGCGCCCAGCGGACCCCTCGCGCCTCCCAGTGCTTCGCTCATCCCCTCCTNCCTTCCCAGCTACCAA | |
| AGACTCGAGCTTGCAGACAGGGACCCAGGGACACCTCNGAGCCCACCGACAAACTCCCGCCTNCTGCTCGGCCCTCTCTGNGGGGGCGGGAGGGGCGCAGGAG | |
| CTGCCCAGNAGTGGGCAGCCGGGCCACACATAGGAGAGCCGGGCAGAGCAGCGCGCAGCCCCTGNCATGCAATATTGAGAGAGGACTTTGTGAGTTTTN | |
| >‘990809A-016.scf” came from CONTIG 73 at offset 0; | |
| “C:\export\EG_DB\990820a\990820A-016.scf’ (51>603) | |
| TATGGAATGATGCTGGCAGGCCTAAGGTTCAAGTAGAATACAAGGGAGAGACAAAGAGTTTTTACCCAGAGGAGGTGTCATCCATGGTCCTGACAAAGATGAA | |
| GGAAATCGCAGAAGCCTACCTTGGGAAGACGGTTACCAACGCTGTCGTCACAGTACCTGCCTATTTTAATGACTCTCAGCGTCAGGCTACCAAAGATGCTGGA | |
| ACTATTGCTGGTCTCAACGTACTTCGAATCATCAATGAGCCAACTGCTGCTGCTATTGCCTATGGCTTAGACAAAAAGGGTGGAGCAGAAAGGAACGTGCTGA | |
| TCTTTGTTTTAGGGGTGGCACTTTTGATGTGTTAATCCTCACTATTGGGATGGATCTTTGAGTCAATCTACACTGGAGTACTCCTTGGGTGGGAGACTTGACA | |
| CCGCTGTTTACCATTTTTGCGAGTCAGCGGACACAGAAGATTAGGAAACAGAGGTGGCGCGCTCGTCGGTGGGGGGCTAGCGCCTTTTTCGCCCCAGCAGTTG | |
| GATGTTCCTTTGAGGATGCTTTACTTTTCCGGCCCTTC | |
| >‘990809A-014.scf” came from CONTIG 74 at offset 0; | |
| “C:\export\EG_DB\990820a\990820A-014.scf’ (61>603) | |
| GCACGAGGCAACGTCATCCGCTATTTCCCCACGCAAGCGCTCAACTTCGCTTTCAAAGACAAGTACAAGCAGATCTTCCTGGGGGGCGTGGACAAGCGCACGC | |
| AGGTCTGGAGGTACTTTGCGGGCAACCTGGCCTCCGGCGGGGCGGCCGGGGCCACTTCCCTGTGCTTCGTCTACCCGCTGGATTTCGCCCGAACCCGCCTGGG | |
| GGCCGACGTGGGCAAGTCGGGCAGTGAGCGCGAGTTCAGGGCCTGGGAGATTGTCTGGTGAAGATGACCAAGTCCGACGGCATCCGCGGGCTGTACCAGGCTT | |
| CAAGGGNNGGTGCAGGGCATCATCATCTACCGCGCGNCTACTTCGCATCTACGAACCGNCAGGGCTGCTCCCGACCCCAGACACGCCATGNGGTGAGGTGAAG | |
| ATCGGCAGACGGACGGCGGGGGGCGTGGCTCTACCCTCGCACGGCGGGGGCTGAGAGCAGCGGCGCAAGGACGCACATGACAGCCCGGGCTGTGCGAGACTAG | |
| ACAGGCGAGGCTTTGAGGCCTGGCAGCN | |
| >‘990809A-018.scf” came from CONTIG 75 at offset 0; | |
| “C:\export\EG_DB\990820a\990820A-018.scf’ (61>603) | |
| AAAGCGCTCAAATTTATGACATACAACAGCTGTGGCCAGACTCTGCTTAAATCAAGAGACAATATGTCTAGCAAGCACTGCTATGGAAAACTGGAAAATTGTG | |
| TGGGCCAAAACAAAACTTGCCAATGGGACTTCCAGTATGATTGTGCCCAAGCAACGGAAACTCTCAGCAAGGCTATGAGAAGGGAAAGGGACTGTGTGTCAAA | |
| TATTGAGCAGAGGGCAGAATCCGATCAAGGGGAATTTGTGGAACATCTTATTTCCAAATGTGTCATTATCCAACATGGGCACATAAACTCATATCTAAACCTT | |
| GTTGTTGAGGGGATTTCTTACTNTCTGCCGCTGGNGTGTAGTCACATTGTGGGACACCTGTGTACTGGGTGGGGATCATTTGTGTGCGNGACGGGTGGANAGA | |
| GGGACGCGNGCGGCGGTGGTGTTTGGGAGAGAGATGAAGAAGGGAGGCGATGTTGGGGAGGCGGGGACGAGGCNGGGCGGTTTGTAAAACAACCCGNGGAAGG | |
| CCCCACCTTTTTGTGGCTTCTATTTCGCC | |
| >‘990809A-019.scf” came from CONTIG 76 at offset 0; | |
| “C:\export\EG_DB\990820a\990820A-019.scf’ (54>593) | |
| CGGCAGCATGTCTCACAGGAAGTTCTCTGCTCCCAGGCATGGGTTCCCTGGGGCTTCCTGGCTCGGAAGCGCAGCAGCCGGCACCGCGGGGAAGGTGGAAGAG | |
| CTTTCCCCAAGGATGACTCTTCCAAGCCTGTGCACCTCACTGGCTTTCTTGGCTACAAGGCTGGCATGACCCACATTTGGGGAGGGAGGTCGATAGGCCAGGG | |
| GCCAAGGTGAACAAGAAGGAAGGGGGGGGAGGCTGTGACCATCGGGGAGACTCCGGCCCATGGTGATTGTGGGCATCGTGGGCTACGTGGAAACACCCCGGGC | |
| CTCCGGACCTTTAAGACCATCTTTGTGAGCATATTAGCGACGAGGGCAAAGGCGCTTTACAAGACTGCATAAGACAGAAGAAGGCCTCACAAAACTGAGGAAG | |
| GCAGACGCGACGCAGAAGAGTTGGAGGATTACAGATAAAAGACTGCAGTATCGGCATGCCAACCAATCGCTGTCTTGGCAGAGAGCCCTATGAGTCAGGACGA | |
| GCCGNGCCAAATGACGGCGGGAGTGGC | |
| >‘990809A-083.scf” came from CONTIG 77 at offset 0; | |
| “C:\export\EG_DB\990820a\990820A-083.scf’ (55>579) | |
| GTTGACCTTGACCTGGGTAACTATGAACGTTTCCTTGATATCCGCCTCACCAAGGACAATAATCTGACCACTGGCAAGATCTATCAGTACGTCATTAACAAGG | |
| AACGCAAAGGAGATTAGGGGAAAACCGTCCAAGTGGTCCCGCACATCACAGATGCAATCCAGGAGTGGGTAATGAGACAGGCCTTAATACCCGTGGACGAAGA | |
| TGGTCTGGAACCTCAAGTGTGTGTTATCGAGCTTGGTGGGACAGTAGGAGATATAGAAAGCATGCCCTTTATTGAGGCCTTCCGTCAATTCCAGTTCAGGGTC | |
| AAAAGAGAGGAACTCTGTTATATTCATGTCAGCCTCGTGCCGCAGCCAAGTTCACAGGGGACAGAGACTAACCCACCAGACAGGGTCGGGACTCAGAGGCTTG | |
| GCTTCCCAGTCTGTTGTTGCCGGGCTCAATCTCTGACCGCAGGAAAGAAAATATGAGTCTGCTGGAACCGACAGGACTGGCCTGAGCTCGCCTTACGGTCCCT | |
| ATGTAGA | |
| >‘990809A-081.scf” came from CONTIG 78 at offset 0; | |
| “C:\export\EG_DB\990820a\990820A-081.scf’ (61>602) | |
| GCACGAGGGATTTTTATTTTTCAGCCTGTTTGTTTTCAAGGTGGAGAATAAAAAACTGACTCTGTTCCAATCTTATTGGTACCAATCAGTATACATCACTTAA | |
| AGCTGTTGCTCCTGAGCTTATATTGAAGTAGCCCTAAGTACCTGGTGAAGTTACATGTATAAGAGAGTTACACATTTGGGGGTTCAGTTGATTTCAACGTAGT | |
| AACATAAACATACTCTAGAACTATACACAAAGATTTACAATTTAAAAACATAATCAGNCACCTTATTACCTGGAAATTTATTACTTTTACTACTCTTCATTTG | |
| CTTCAGACAATACATATTTCATTCCTTATAATCTAGAATTCAAGAACCAGGACTCTAATCTTTTTCCTCTCATCTCTTATCTTTCTAAAATTGCCTGGGTAGA | |
| GATCAGGTATAAATATTTATATTAATTGATTATTATGGTAGGGGGAGGCAAGAACCAACCATACAGGATCATGCTACGATTTAGACATAGNCAAAACAGAGAA | |
| GGANNATATTTTTAAGGATATATAT | |
| >‘990809A-077.scf” came from CONTIG 79 at offset 0; | |
| “C:\export\EG_DB\990820a\990820A-077.scf’ (55>571) | |
| GTGGTACTAACCTTACTTCCCTTAGTGTTGACCCGGAGAAGGAACGATGGTGCTGGATCTGGATTTGTTTCGGGTGGATAAAGGGGGGGAACCCAGCACTCAT | |
| CCGAGAGTCGCAGGAGAAGCGCTTCAAGGACCCGGGACTGGTGGACCAGCTGGTGAAGGCAGACAGCGAGTGGCGACGATGCAGATTTCGGGCAGACAACTTG | |
| AACAAGCTGAAGAACCTATGCAGCAAGAGAATTGGAGAGAAAATGAAGAAAAAAGAGCCAGTGGGAAATGATGAGTGCATTCCAGAAGAGTATTAAATCTCGA | |
| TGACCTCACTGCAGACACTCTAACTAACCTGAAAGTCTCACAGATCAAAAAGTCCGCCTCCTCGNCGACGAAGCCTCTGCAGGTGACGCCGAGCGGATAAAAC | |
| GGGAGCTGACGCCTGAGAGCGTTCGGAGATCGGGACCTCTGCACCGNCGNGCCATCACGAGACAGATGCGACACAAGANAGAGATTGGGGGATGTGAGNCAGA | |
| GA | |
| >‘990809A-085.scf” came from CONTIG 80 at offset 0; | |
| “C:\export\EG_DB\990820a\990820A-085.scf’ (27>51) | |
| TATCTAGTGGTTTCCCCCCGCTTCT | |
| >‘990809A-082.scf” came from CONTIG 81 at offset 0; | |
| “C:\export\EG_DB\990820a\990820A-082.scf’ (61>456) | |
| GCACGAGGCTCTGGCGTCCCGAATTGCGTCTGTTTTTTCTCACAGGCTGTGTCCCGTCCGCTGGCCCAACACCTCAGGGGAACGATGGGCCGCAGAGTCCACA | |
| GCCACTGCCGGCATCACCGCGGAGCTGGGTTCTGCCGACAAAATTGAAGAAGATGCCCCTGCTCCTTCTACTTCTGCAGATAAAGTGGAGAGTCTGGATGGGG | |
| ATAGTGAAGCTAAGAAACTCTTGGGATTAGGACAGAAACATCTGGGAATGGGTGATATTCCTGCAGCTGGCAATGCCTTCCAGGAAGCAGCTAGNCTTTTAGG | |
| TAAGAAGGAGGGAGAGACAGCTAATGAATGTGGAGAAAGCCTCTTTTTTTATGGGAATCGCTTTGGAGTGGGCAGATGGGAGATGGG | |
| >‘990809A-082.scf” came from CONTIG 82 at offset 0; | |
| “C:\export\EG_DB\990820a\990820A-071.scf’ (49>598) | |
| TAGGCGCAGATCACCCTCGGCCTGGTCAAGTTCAAGAACCACAGGCCATCCAGACCGTGCGCGCCCGGCAGAGCCTGGGGCCGGGGCGCTGTGTGCTAGAGCG | |
| CGGGGGCGCGCCTGGCCTGCCCGAGGTCGTGCCAGCTTCTGGGCCCCCATCGGCTCTCGGAGCGTGACTGTGCTCCTGACTGTGCTCCGAGTGGCGAGGCTGC | |
| GGCTGCGGACACCCCCGTGGAGGAGGCGTTGGAGTCACGGGTCAGCGGGAGCCAGCAAGGAGGGGGACAGCCACGGAGAGCTGAGGATGAGGATTCGGCCCCG | |
| GGCGCTCAGGCTCTCAGGACGTGCCTGTCCCAGCCCAGCCTCTGAGCTCCAGCAAGCCCCGACCGCTGCGCCCTATTGCTACTCTGCAGATGTCCGACCAGCT | |
| TGGGCTGGGCTGCAGAGGCGGACCCGGATCGTGACCACATTTAGACTGGAGCCGCTGGGACACGGACAACCCCAGCTAATTGATCACGAGCCATCTGGGTGTG | |
| GGCTGGTCCTCCGACCCCCCTCGCCCTTTTACATC | |
| >‘990809A-070.scf” came from CONTIG 83 at offset 0; | |
| “C:\export\EG_DB\990820a\990820A-070.scf’ (59>543) | |
| GCACGAGGGTTTCACATAAAACACAGGGAAAAATGTCACGCTTGAAGGTGGCCCTTCAGTTCTCCTAGTGAAAGGGCATGTAGCAACAGTACCTTGTGCCTGG | |
| CCATCTTTACTCCAGCTCACGTGCTCTGGTACCTCATTGTGTCCTCACAGCAGCCCTGGAAGGCGGGAGGGGCGCTACTGCCATCCTTGTGTGTGTGAAAACT | |
| TGAGACCGAGATAAGGCCAGCGACTTGCCTGACTAAAGTTGGCTAGGTTTGCCCAAGCTGTGCCATCATTCCACACTCAGCAGAGACGGGGAGAGCACCTGTG | |
| GGGCAGAGACTGAAAAAAGTCCCCAGNTCCGCTTTGTCCTCCTGGGCCCGCGGCTGCCCTGCCCAGCCAGGTACTGTTTTCTCTTCACGATGATAAACCTGAC | |
| ACCAGTCTTGTGCGACACAGGGGCCTGGCTATCTCTGCATATCACAACATACTGGCTCCTGGGGCACGGNAAGA | |
| >‘990809A-080.scf” came from CONTIG 84 at offset 0; | |
| “C:\export\EG_DB\990820a\990820A-080.scf’ (61>603) | |
| GCACGAGGCYFGCATCCAGTTTGACTACAAAGGAGCTTCATCTTTCAAGATAACCCGTGGAATTGAAGCAGTTGGTGGTAAATTAAGCGTGACGTCAACAAGG | |
| GAAAACATGGCCTATACTGTGGAATGCCTGCGGGATGATGITGATATTCTAATGGAGTTCCTGCTCAATGTCACCACAGCACCAGAATTTCGACGCTGGGAGG | |
| TAGCTGCCCTTCAGCCTCAGCTAAGGATTGACAAAGCTGTGGCTCTTCAGAATCCACAGGCTCACGTCATTGAAAATTTGCATGCTGCCGCTTACAGAAATGC | |
| CTTGGCTAATTCCTTATATTGTCCTGATTATAGGATTGGAAAAGGACACCAGTGAGTTACTGACTATGTACAGAATCATTTACAGTGCAGGAGGCTTTGTTGA | |
| CTGGGGNGTCATCTGCCTAAGCAGTGCTGACACTTCTACATAAGGGGGCTTGTTATTGGGCAAGCCAGACCATGAGGAAATGAGACAGAGAGACGCCGCCATC | |
| CGTTTGGCAAAGCACCTGAAGCAAACAA | |
| >‘990809A-076.scf” came from CONTIG 86 at offset 0; | |
| “C:\export\EG_DB\990820a\990820A-076.scf’ (56>562) | |
| TTTGATTGGGAGGAAAAAGACAGGCCAAGAAGCTCTCTCTCAAACAACCATCTCATGGACCCCATTCCAGGAAAGCTCTGAGTATATCATTTCATGTCATCCA | |
| GTTGGCATTTGATGAAGAACCCTTACAGTTCCGAGTTCCTGGAACCTCTGCTAGTGCCACCTTGACGGGCCTCACCAGAGGGGCCACCTACAACATCATTAGG | |
| GAGGCAGCAAAAGACCAGCAGAGGCAGAAGGTTCGCGGGAGGGGGGGTACCGNGGGCATTCTGTTGACCAGGGCCCGAGCCAGCCCACACATGACTCCTGCTT | |
| CGACCCCTCACGGCTCCCATATGCCATTGGAGAGGGTGGGAGGGATGGCTGTTTGGCTTTACTCTCGGCGCGCTTAGCTTGGCGTGGGGATTCCATGCGATAT | |
| CTATGTGCCTGACAGGGAGAACTCAGAAGAGGAAGGGACGGAGGGAGATGGCGAGAGGCGCCGGCCTGAATGAAAGAGATAGGTGACCAGAGCCAG | |
| >‘990809A-005.scf” came from CONTIG 86 at offset 0; | |
| “C:\export\EG_DB\990820a\990820A-005.scf’ (5>15) | |
| ATTTTCGCGGN | |
| >‘990809A-011.scf” came from CONTIG 87 at offset 0; | |
| “C:\export\EG_DB\990820a\990820A-011.scf’ (49>63) | |
| TTTTGTTTAACACCT | |
| >‘990809A-023.scf” came from CONTIG 88 at offset 0; | |
| “C:\export\EG_DB\990820a\990820A-023.scf’ (1>14) | |
| GCCGCATGAAACCC | |
| >‘990809A-041.scf” came from CONTIG 89 at offset 0; | |
| “C:\export\EG_DB\990820a\990820A-041.scf’ (5>17) | |
| ATTTGAAAACCCC | |
| >‘990820A-045.scf” came from CONTIG 1 at offset 0; | |
| “E:\SEQUENCE\export\EG_DB\990820a\990820A-045.scf’ (58>535) | |
| AGGCACGAGGCAGAGACGCAGCAGCGGCTCCCTCTGCCCACACCCACCGCGCCCTCGCGCTCGCCTCTCCTTCCGGAGCCAGTCCGTGCTACCGCAGGCGCCC | |
| AGTCCACCACCACCCTCTGCAGCCATGTGCACCAGGTCCGTGTCCTCGTCCTCCTACCGCAGGATGTTCGGCGGCCCCGGCACCGGAGTCGGGCGAGCTCCAC | |
| CCGGAGCTACGTGACCACATCCACCCGCACCTACAGGCTGGGCAGGGCGCTGCGCCCCACCACCAGCCGCACCCTCTACACCTCGGCCCCGGGGGGGCGGGGC | |
| CCCCAGCGCTGCCTGGCCGTGCGCTGCGGGCGGCGGGCCGCGTGGGGGGGTGGAGAATTGGGGGATTCCGGGGCCGACGCCTCACACGATTCAAGACACCGAC | |
| ACGGGAGGGGGGCGCAGACCCAAGCCGCTCGCAAATAAGGACAGGGGCTCTGAGAGAAAAAGAACG | |
| >‘990820A-013.scf” came from CONTIG 1 at offset 3; | |
| “E:\SEQUENCE\export\EG_DB\990820a\990820A-013.scf’ (54>434) | |
| CCCGCGCCAGAAACGCAGCATCGCTCCCTCTGCCCACACCCACCGCGCCCTCGTGTCTCGCCTCTCCTTGCGGAGCCAGTCCGTGCTACCGCAGTCGCCCAGT | |
| CCACCACCACCCTCTGCAGCCATGTCCACCAGGTCCGGGCCCCGGGCTCCTACCGGAGGATGGCGGGGGGCCCGGGACCGGAGGGCGGCCGAGCTTCACCGGG | |
| GCTACGTGACCACATCCACCGGACCTACAGGCGGGGAAGGGCGCCGGGGCCCACCCCCGCCGGACCCTTACACCGGGGCCGGGGGGGGGGCCCCCCCCGCCCC | |
| GGGCCGGGGGCGGGGGGGGGGGGGCGGGGGGGGGGGGGGGGGCGGGGGCTTGGGGGGCGGCCCCCAAACCAA | |
| >‘990820A-002.scf” came from CONTIG 2 at offset 0; | |
| “E:\SEQUENCE\export\EG_DB\990820a\990820A-002.scf’ (62>518) | |
| GGGCGGGGCGTTTTTTTTTTTTTTTTTTTTAGCGGGGTGGCTAGGGGGCTTCTGGAGTCTTTTATTTTGTAAATATATAAGATACCTTTTTTTTGTTTTTATT | |
| TGTTGAAAAGAAAGCTTTCATTGGCTTTTTATGGTGGGGGACATGGGGGGAAAGGGGGGAACCCCATGCATGGCGCACTCCCCCCCAGGGGGGGGGGGGGGGG | |
| GAAAGGGGCGGAAAACCCCGGGGCTCCTGGTGCAAAAGCTTCCCGGGACAGGGTCCAAATGCAGGAACCAAAGGGGCCGGGGAGGCGGGGGGGGAACCGCGGA | |
| GGGGTTCACTCTTGGGAGGACATAACATGATTCGGCCCCAACTTCCCCACCCGGGGTAAAAATTTCTAAAACCCCCGGTTGGACTGGGGGGCGGGGGGGCCGG | |
| GCCCGGGTGGGTGGAAACAGGGGCCCAAAAAAAAAAGCGACC | |
| >‘990820A-043.scf” came from CONTIG 3 at offset 0; | |
| “E:\SEQUENCE\export\EG_DB\990820a\990820A-043.scf’ (58>460) | |
| TGGCACGAGGGAAAAGGTGGCTGGTGGTGAGTTGCTGGGTTCCTCCCTGGTGCCTACTGGGTTTTCCCAGGGTGGCTGGTTGGGGCGGAGGGCGGGGCCCCCC | |
| CGGCCCCCTTTTGGGAAAATGCCTGGGACCCCCCTTGGCCCCCTCCCTGGCGCCTGGGTGGGAAAAAAAAGGGAGCAAAAAAGGCCCCCGGCGGGGGAAGAAT | |
| GGGGCCCCGGTTGGGGGGGCCCCGGGGGAAGGGCCGGGGCCCCCCTGGTCCCCCCCTGGGCCCCGGGTGGGGGGAGAAAAAGAGGCCCCGTGGGGGCGGACGG | |
| GCACCTGGTTGGGGGCTCCCGGGGACCTCCCTGGAACCCTAAAGGAATTTGGTGGGACAGGGGGGGGGGGGGGGGGGGACGGGCCCGGGCCAAA | |
| >‘990820A-035.scf” came from CONTIG 4 at offset 0; | |
| “E:\SEQUENCE\export\EG_DB\990820a\990820A-035.scf’ (60>530) | |
| GCACGAGGGTTTTTTTTTTTTTTTTTTTTTTTTTTTTTTTTTTTTTTGGGGTTAACAATTTTTTATTAGGGGGGAGGTTCGGAACAAGGAAGAAAGGGGGGGG | |
| AGGAGGGAGGGAGGGGGGGGGAGGAGAACACCAAGCCGGGGTAGGTGGGCCACGGCCAGAGGGAAAAAGGGGGCCCCGGGATGGAAGGGGAGCGGGGAGGGGG | |
| ACGGGGGGCAGGCAACAGGAGGGGGACACGGAGAGGGGGGGGGGGTAGGGGACAAGGGGACCCCACATCCCCGCCGGTCCAATGGTAACAGAACAACCAAAGC | |
| CCCGGGGGGGACAAGGGGGGCAACTTGCGTTGGAACAGGGTTAAAATCGGGGAAGGGTGACCAACGCCCTTGGCGGGGGGTGGAGAAAAAAAAAAGAAAAAAC | |
| CCCCGGACAAAAAGACCCCATTATGTTGGTGGAGAGGGGAAGTGGGGGGGAACCCACGN | |
| >‘990820A-014.scf” came from CONTIG 5 at offset 0; | |
| “E:\SEQUENCE\export\EG_DB\990820a\990820A-014.scf’ (56>446) | |
| GCACGAGGGTTTTTTTTTTTTTTTTTTTGGGTACCAAATTACTTTATTTGGAAGGAATGGGTAGTAAAGAAAGAACTTAAGTTGATGTTTTGTTACAACTTAT | |
| AGAAAAGATGAAGGTAACCCAAATATGGCTGCACTGCCTTGGGGACCAGGGAAGGCACCCCTGTGGGTGTGGGAAACCAGCCTGGGGCTTTAGTTTCACTGGG | |
| GCCAGGGTGTGCTTGGGAAAGGGGACCTCTGCCTTGCCAGATCCCGGAGCCCCCCTCTTGGGCCAGGTGGTTTTGGGGGCACCCATTTTTTGGTGGTTTCCCC | |
| CGTCTATTAAGGAAGGGGTTAAAGAAAACACAAAGGGGTTATTTTTTTAGGGGGGTGGGGGGGGCCGGGGGCGGTTCCACCT | |
| >‘990820A-011.scf” came from CONTIG 6 at offset 0; | |
| “E:\SEQUENCE\export\EG_DB\990820a\990820A-011.scf’ (60>496) | |
| GCACGAGGGTTTTTTTTTTTTTTTTTTGCTGTACTGAGCAGCATGTGGGAACCTTAGTTCCTTGGTCCAGGGGATTGAACCTGGGCCCCCTGCAGTAGAAGTA | |
| CAGAGTTAACCACTGGAACTGGTACCAGGGTATTCCACACATAGATTTTAGGAGCTTTGTGCTGTATGCTTTTAAATGCTGGCATTCCAGAGATAACATGAAG | |
| ACAAACTGGGTTTGGAGAGTCTCATGATCTGTTTTCCCCTCCGGGGCTGGCCATAGAAGACTGAAATCCAGGGAAGAAAGATGCTGCAGCAGCAGAGAGCGCT | |
| GATTCAAACTACCTGCTTACAGGCATGCTCGACGGCAGAGGCAGGCGGGGCTAAACAAACCGGGGACTATGTTTCTAATTGAAATACGTGTGGGCTTTCCTTG | |
| TTGGCCGGGGCAGGAAGGAACGAGTGG | |
| >‘990820A-003.scf” came from CONTIG 7 at offset 0; | |
| “E:\SEQUENCE\export\EG_DB\990820a\990820A-003.scf’ (61>525) | |
| GCACGAGGGTTTTTTTTTTTTTTTTTCTCATTAAACTTTTTTTAATGGGTCTCAAATTCTGTGACAGATTTTTGGTCAAGTTGTTTTCATTAAAAAGTACTGA | |
| TTTTTAAAAACTAATAACTTAAACTGCCACACACAAAACATATGGTCCACAAAAACATTCTCCTTTCCTTCTGAAGGTTTTACGATGCATTGTTATCATTAAC | |
| CAGTCTTTTACTATTAAACTTAAATGGCCAATTGAGACAAACAGTTCTGAGACCGTCCTTCGACCACTGATTAAGACTGGGGTGGCGGTATTTGGGGATATAT | |
| TTCATTTACCTTTTGACTTTTTGGCAGACTGGGACCTTCCACCCCAGCGCCTCCTGCCATGTTTTGAGACCCCCGCGATGTTTGTTAGTCCGCCAGAAACGCC | |
| AGGGAGGAATAAAGAACTTTGCCCATGGGTTGCCGACCTTAACGTGGGGGGGCCC | |
| >‘990820A-038.scf” came from CONTIG 8 at offset 0; | |
| “E:\SEQUENCE\export\EG_DB\990820a\990820A-038.scf’ (50>535) | |
| AGGCTCGAGTTTTTTTTTTTTTTTTTTGGGAAAACAAGAAATCAGTTTATTTTCATCATTTTCTTGGCATTTGGAAGGTACTCCCCGGATGGACATCGCTTGG | |
| GCCTTGAGATTCTTTGCCATAGACCCTAAACCACCACACAACTGCAACCAACCACTTTACGAGGGTTCCCCTCTCTGTCAATTTTACAGAGGCCTACCCCTTC | |
| CCCTAGTTTCTTTTTGCCATCAACCTTAATAAGGTGATTTGAAGCTCAGCACAAAGGGCCCTCACCAACTTGACATACATTAGCTCATCACAGTGGGATGCGA | |
| GCACACACAGAGGGGCTTGGCGCTTGGGCTAAGCTTCGAGATTCCGATTCCAGGCTAGCCATGGGATGAGGCGCCTCAGCCCCTGCGAAAGATAACGCCTTAC | |
| ACCCCGCACATGCCTCCTGCCTGGGGGGGTAGGGGGGGCAAACTGATGCCCGACCTCCCCCCGCGTCCCGGTGN | |
| >‘990820A-072.scf” came from CONTIG 9 at offset 0; | |
| “E:\SEQUENCE\export\EG_DB\990820a\990820A-072.scf’ (9>114) | |
| GGGGGGGGGGCCGTTTTTTTTATTTTGTTTTTCTTTTTTTTTTCTTTTTTTTTTTTTTTATTTTTTTTTGTTTGTGTTTGGTTTTTTTTTTTGGTTTTTTTGT | |
| TG | |
| >‘990820A-096.scf” came from CONTIG 10 at offset 0; | |
| “E:\SEQUENCE\export\EG_DB\990820a\990820A-096.scf’ (55>399) | |
| GTCTCGAGTTTTTTTTTTTTTTTTTTCAGTCCTGGATGAGGCTGCAGCTTTGGCACATCATCCACTGTCCCCAGCCATGATGTAAAGGGCACAGACTTTGGGG | |
| TFPGTCATAGGATCCCTTGACAAATTCGATGTAGAGTTGCCTGGGAAGGTGGACACCTCGCCCTGGACGCTCAGCTTCCCCTTCCGGATGCTCATCGGGATGA | |
| TCTCATCATGGGCAGTACTGGGCCCTACAAGATCAAAGATATCCAGGCCCTTCACCACCCCGTGGCCGGTCAACCGCACGTAAAACACCTTTTGTTGGGACGG | |
| GCCAAGAAGACCCAAAGAACTTAACACCGCACGTAG | |
| >‘990820A-062.scf” came from CONTIG 11 at offset 0; | |
| “E:\SEQUENCE\export\EG_DB\990820a\990820A-062.scf’ (56>435) | |
| AAGCCTCATGTGTATGAGTGTTTTCCCCTCAGACTGCACACCTTTCATCTCTTGTAGCTGCTCTGGCGTAGAGTTGCAGAGATCTTCATGGTGAGCAATTAAG | |
| AAATTTTAGTGAAAAGTAGATAACAATTTCAGAAATCAGTTTCTCTGGTCTTTTGTAATACTGTTGGCTTCCCATGGCTTTTTTTGGAGTTGTTTATTGAATA | |
| TGTGGTTTTGACAGCCTCCTCATTACAGTTTCTTAAATGCATACTGGTTTGTAAAGAATTATTGACGTTATTCATTCCATTTATGAGAAAAGAGAGAACAGCT | |
| AATAAAACTTATTGGAAAATCGGAAAAAAAAAAAAAAAAAAACTGAGGGGGGCCCGTACCCCATCGCCTT | |
| >‘990820A-050.scf” came from CONTIG 12 at offset 0; | |
| “E:\SEQUENCE\export\EG_DB\990820a\990820A-050.scf’ (60>520) | |
| GCACGAGGTCGAGTTTTTTTTTTTTTTTTTTAAACACTACAGAGTGCAAATCAGGTTCTTCACAATAGATTGAGTATTAAGCAGTTCTTGAAAGAATGAGGGG | |
| GGAAGAAGAAAAGCCCAAGTGAATAAAACATTGAAACTATTCCCCTTGAAAATAAATTCTAAAATGATGCGGAATGTGAAATAAGCTTTAAACATAGGGGATC | |
| CGAGTTTATAGATAGAAACAAAAAGTAGTCCTTNATGAAATAAGGTTACAAGAACATGTGGCTGTTTTTCCCTGTTATAAGTGGAAGCGAGAAGAGACGAGTT | |
| TGGGACGAAATAGCGACTCAGATCACTTTCCTTCAGAGCTGACCCAAGCAGCTGAGTGCCGAGCTGTGAAGGAACCAAGCAACGGGCCGGGGGGACGAACGCG | |
| GGCCGCTGCAGAACACGGGCGACATAACAAGGGGGCTCGAGCTGTGTT | |
| >‘990820A-001.scf” came from CONTIG 13 at offset 0; | |
| “E:\SEQUENCE\export\EG_DB\990820a\990820A-001.scf’ (11>536) | |
| GGGCGGGCGTCTTTATATTTGGATCCCCCGGGCTGGGGGGAGCGAGGCGGGAACGGGGCGTTGGGTCTGTCGTTTGGGCCGCCCAGACACCTTTACTGTAAAG | |
| ATGGTCAACGTACCTAAAACCCCCAGGACTTTTTGTTGGAAGGGTGGAAAGCATCAGCCTCACAAAGTGACCCAGGATAAGAAGGGGCAAAGGATTGCCTGGG | |
| TGCCCAGGGAAAGAGGCGCCTTGGATCGGGAGCAAAGGGGGTCCCGGGGGGCAAAACCAAGCCAATTTTTCCGGAAGAAGGGGTAAACCACCAAGAAAAAGGT | |
| GGCGAGACTTGGATGCGTGGGGCCCAACTTGCGGATCCAAAAGGATGCTGGCCTTTAAAAGGGGCGGCCTTTTGGAATTGGGGAGAAAAGAAAAAAGGGCCCA | |
| GGTTCCCTTTAAATTTGGGTTTTTTGTTTTTTTTTGAGAAAAGGGGAGTTAAAAAATGGTTGTGGGTAATTTGGGTTTTCTTTTAAAAAAAAAAAAAATGGGG | |
| GGGGGCGGGCTT | |
| >‘990820A-079.scf” came from CONTIG 14 at offset 0; | |
| “E:\SEQUENCE\export\EG_DB\990820a\990820A-079.scf’ (54>478) | |
| TTTTTTTTTCGTAAAAACCTGTTTTTAATTTTGTATAAAACAAAGGGTGGCCTCCGCCCCAGGGGGCTGTAGGGAGAATTCAAGCTAGACCAGCTGGNGGTGG | |
| GGGGTCACAGGCCTACCTCGGGGGGGGGGACGGGCCCTGGAGGGGGGACAGGGAAGGCATGGCAGGGGGCCCAAGGCCCACAGAGCACCCCGGGCCGCCCCCC | |
| CGCCCCGGGGGCAGGAGACTAGCCCCTCCGGGGGCCCCCCTTGGGGGTGCGGGGGGGGGCCCCAGGCCCCGCAGCCTCTAACAAGACGAGAGAAAAGGGGGAA | |
| GGGGGGAAAGCACCAGGGAGGGAGAACCCTGGGGCCCCCGGCTGCGGGGGGCCGCTTCCCCCTACAACCCCCAGCTTCTGGCCAGGCGGCGCACAACTGCACA | |
| GGCCCTGTGGGC | |
| >‘990820A-015.scf” came from CONTIG 15 at offset 0; | |
| “E:\SEQUENCE\export\EG_DB\990820a\990820A-015.scf’ (60>454) | |
| GGACGAGGGCGGGAACGTGCTGGACCCTCTGTGCCAGCTGGGCAACCCCCAGCYJTCGGGTTTTTTCGCACCAACTTCTTCATCCAGGGTGGGTGCGGGGCCG | |
| GCACCGGGCCCAGCCCGGAGGGATACCCGTGGCAGGGTCCCGGATCCCCCATGGAAGGATGGACCAGAAGTGGACCCTCAGGAAACTACCTCGGGCGCGGTTA | |
| CCATGTGCGGGGGGCCGCCGGGGGGGCGAGGGGGGAGGAACGGGTCCAAAAAGGAGGAGGGAACACAGGAACGTAGGGATAAAAAAGCCAGATTTTAAGGGGG | |
| CCTCGGGGCAGTGGGACAGGGAGAACTTTTAATTTCCAGAATTTGTTTCCTAGGGGGAAGGGGGCCGGGGGGGGTCGGGGGGGGGGAC | |
| >‘990820A-009.scf” came from CONTIG 15 at offset 368; | |
| “E:\SEQUENCE\export\EG_DB\990820a\990820A-009.scf’ (60>430) | |
| GGACGAGGGGGGGGTCGGGGGGGGGACCGGGCCCTGCTGGGTCCTGCTTGGGCCCATTGGTCCCCCTTGGGTGCCCGGGGGCCCCCCTGGAACCCCAAGGGCC | |
| CGGGGGGGGAAGGGGGGGGGAAAGGGGGAAAAGGGGGACAAGAGGGAAATAAGGGGGGACCGGGGGTGTTTTTGGTGTTCAGGGGGCCCCCCCGGGCCTTCCC | |
| GGCTTTGCTGGGGAGCAAGGGGTCTTTTCGGGACCTTTGGGTGCTGGTGGGTCCCCGCCGGTGCCCCCGGGGTTTTTGCGGGGTNTTCCGGGGAAAAAGGGAC | |
| TGAAGGGCTGGCCCAGCCCCTTTGGGCCCGCGGGGGCCAAAGGCGGGATGGGGATGGTGGGG | |
| >‘990820A-019.scf” came from CONTIG 16 at offset 0; | |
| “E:\SEQUENCE\export\EG_DB\990820a\990820A-019.scf’ (60>343) | |
| GCACGAGGGGGGGGCGGGGTGCCGGGTTTGGGGCGCGACGCCGAAGGCGTGGGAGGAAGGCGGAGTGGGGGCCGGCCGGGGGCCGTTTTGGGAACGGCAACGA | |
| ACAGGAAGTTGGGGCCCCGCCGGGCCCGGGGCGGACGGGGGGGGGGTGGGGGGGGAAGGGGGGTCGGGGGGGGGCCCGGCCCCTGGGGGGGTTTGTGGGCTTG | |
| AAATTCCCAAAGGGGGGGAACCCTGGCCCCCCCCACATAATTCTTCCTGGGGGTGCAAGGGGGGGGGGGGGCGCCG | |
| >‘990820A-094.scf” came from CONTIG 17 at offset 0; | |
| “E:\SEQUENCE\export\EG_DB\990820a\990820A-094.scf’ (59>475) | |
| GGGGACGAGGAGAAGCCATTAGAGGTTGGGCCCCATGCACAGATGGAATAGACTACACCAATCATTCAAACACCCTCCCTTCTTGGTTTACTTTGCACGACTT | |
| GCAAATGAGGAGAAGAAGAAAAAAATCGCTGCACCCCAACCAAGGACACTGAGTGGCAGAGCAAACCTGGCACTTTGCGTGGAAGAAGATGCACCTGAAATTC | |
| TGGCAAAAAGCAAGACCGGGGGCCTGATGGGAAGACATGGTTATGGGCCGGAGCCCCCGGAGCACATAAATGGGGGGGACCAGAATCAGCCCTCTTTAAAGTG | |
| GGGGATGGAAAAGTGTATGGGACTGGAAACCCGGGGGCTAAGGGGGGGGGTGGGGGGGTAAAGGTTTTAGGGGGGGGGAAGGGGGGGGTTTTCGGTGGGGGGC | |
| GAGCCC | |
| >‘990820A-005.scf” came from CONTIG 18 at offset 0; | |
| “E:\SEQUENCE\export\EG_DB\990820a\990820A-005.scf’ (118>329) | |
| TGTCTTTCCACTTCAGGGATGACCCTTTCCCAACGACGTGAATCTCGCAAGGCTTCCTCCCTTTTAACGAATGGGACGTCTGGCGGGAGAGTCCGGGGGTTCA | |
| GCACACGCTTGGAGGTGGTGGGCCCCATGGGGACCTGTACTGGCTTGGCCGGCTGTGTGTGTGGACGGCGCTTTCGGGGCGACTTTGGGGGGGCCCAAAGTCC | |
| CCA | |
| >‘990820A-017.scf” came from CONTIG 19 at offset 0; | |
| “E:\SEQUENCE\export\EG_DB\990820a\990820A-017.scf’ (60>389) | |
| GGCACGAGGAAGTACTTCTTGTCCTGGGAAAATTCCCATGGGATGGGAGGGACGCCTGGGTAGGTCTTACAGGTCTATGTGGGTGCGCAAAAGAGGTCGGGAC | |
| GGCGACCTGAAGTGAACTTTCACATTAAAGGCTGGTCACCCATTGTTTGCATACATTTTTATGGTTCTTTGAGTTTTGTTAAGGTTCTTATGACTTTTTTTCT | |
| GGGGGCTTTTCCTGGGTGGGCTAAAGAGGGTTTAAAGCCGTCCGGCCTGGCAATGCCAGAAAGACCCCAGGGTTCAATTCCCTGGGGTTGGGAAAGATCNCCC | |
| TTGGAAAAGGGAAATGGGAAA | |
| >‘990820A-008.scf” came from CONTIG 20 at offset 0; | |
| “E:\SEQUENCE\export\EG_DB\990820a\990820A-008.scf’ (55>287) | |
| AAAACATCACGTTGGGAGAACCGCCTGGGGTTTTGCACTCGGGNGGGGGGTATTTTGGGGACCTTTACTGGGCAGCTCCTGAAAGGCGAGACACTTGTGAACA | |
| TTCAAGTGAAGCAAAAGCTTTCATGATTACAGTGCAGCAGCCGCCCCGAGCCCCGTGCTTGGCAACATTCCCCCAAGGATGGGAGGGCGGGAGGGCCCATTCC | |
| TCCGGTTTTTTTTTTAGCTTTTTTTTA | |
| >‘990820A-004.scf” came from CONTIG 21 at offset 0; | |
| “E:\SEQUENCE\export\EG_DB\990820a\990820A-004.scf’ (209>270) | |
| CCGGGAACCCAAAGGGATTTTTTCTGTTTGCTGAGATTATACTATGCCTGGAGATTCTGATC | |
| >‘990820A-016.scf” came from CONTIG 22 at offset 0; | |
| “E:\SEQUENCE\export\EG_DB\990820a\990820A-016.scf’ (54>535) | |
| GTCCACAGGCGTGCTGGGCTCCCCGCTCATCTCACTGCTCTTCTGGATCCTCATCTGCTTCTCCATCGCGGCCCTGTTCACCAAGCGCTACAGCATCCGCCCC | |
| CTCATCGTGGCGCTCATCCTGCGTTCCATCTACTACCTGGGCATTGNGCCCACGCTCAACATCCTGGGTGCCCTCAACCTGACCAACAAGATCGGGGTCGTGG | |
| GGAGCTTTGTGGGCAACCGTGGCACCTTCATCCGGGGCTATAAGGCCATGGGCATGGACATGGAGTTCCTTACCACGTGGGCTACATTCTAACGGGGTGTTTT | |
| GGCCCTTTGGCCCCGAGCCTTTTACGGATTCTGTTTTTACGCTTTTACGGGAAGGACCTGGAAAAGGGAAAAAAAGGGACCCGAAGGCCGCCAACTGCTGCCG | |
| CCGGTGGCCCCATCGGCTACCTTTTCTTTGGGGTTCCTTCCAAGAACATTTATCTTGGGGGCCGGTGCC | |
| >‘990820A-033.scf” came from CONTIG 23 at offset 0; | |
| “E:\SEQUENCE\export\EG_DB\990820a\990820A-033.scf’ (54>544) | |
| CGCCGCGCACGCCATGGGAGGCCGGGGACTGACGACGAAGTCATACGGTAAGCGTCGTCCTAATCGGATGGAGGATGGTGGCTGGGAGATGAATCGGAAGAAT | |
| TATATCTGCTGGTGAAAAGTTTCATTAAATGGTGCAACTCAGGATCTCAGGAAGAGGGATACAGCCAGTACCAACGTATGCTGAGCACGCTGTCCCAGTGGGA | |
| ATTTTCAATGGGCAAAACTTTGCTGGTATATGATATGAATCTCAGAGAAATGGAAAATTATGAAAAAATTTACAAGAGATAGAATGGTGCATTGCTGGGAGCC | |
| CATGAAAAAATTGCTGAGTGCAAAAGCCAATTCTTCAGCAAAACGATACAAAAAATGGCAGATAGATGCACTGGCAAGGGATCCACACATCAGCAGGCCCAAC | |
| GCAAAAGACTCGGGCCTGGAAAGAATAGACTCTTAAAATTTAAGAGGGNGAAGAAACGGAAAGAACGAACGCCCAGGCTC | |
| >‘990820A-020.scf” came from CONTIG 24 at offset 0; | |
| “E:\SEQUENCE\export\EG_DB\990820a\990820A-020.scf’ (60>504) | |
| GCACGAGGCAGAGGTGCAACTTTCTTCGGTACGTCCCGAATCCGGGTTCATCCGACACCAGCCGCCTCCACCATGGCCGCCTAAGTTTCGACCCCAACGAGAT | |
| AAAAGTCGTGGACCTGAGGTGCACCGGTGGGGAAGACGGTGCCACGTCTGCCCTGGCCCCCAAGATCGGCCCTCTGGGTCTGTCTGCAAAAAAGGGCGGTGAT | |
| GACATCGCCAGGGCAACGGTGGTTGGAAGGGTCTGAGGATTACAGGGAAACTGACCATTNCAAACAGACAGGCCCAGATGGGGGGGGCCTTTGGCTTGCCTTT | |
| GATCTTCAAGCCCCAAGGACCCCCAGGAAAGAAGAAAAAAAAAATTAAACCAAGAACATTTTTTTGAGAATGAAATTGCCGGGGAGCGTTGGGGTTTATTGGA | |
| ATTTGGGACATAGGGAATCGGGACGGCGGGGGG | |
| >‘990820A-010.scf” came from CONTIG 24 at offset 0; | |
| “E:\SEQUENCE\export\EG_DB\990820a\990820A-010.scf’ (60>532) | |
| GCACGAGGGTGTGTTCTGGGGAGAGGAGCACCACTCCGGACATTACTGGCTACATAATCACCACCACCCCAACAGACGCGCCAGGAGGCGATACGTGCTCTGG | |
| AGGAAGAGGGGCATGCGGATCAGAGTTCCTGCACCTTTGAAAACCTGAGTCCGGGCCTGTTGTACAATGTTCAGTGTTTACACTGGCAAAGATGACAAGGAAA | |
| GAGTCCATATGTCTGATACCATCTTTCCATCTGTCCCTCCTCCCAGTGATTGGCGATTTACCAATGTTGGCCCTGACACATGCGTGGCACCTGGCCTCCACCT | |
| CATCCATCGAACGACCACCTCTGTTTCGCTCTGGCTGGGAAAAAGAGGGAGGTGCCGGCTGCCTTTCTCCTCAACAACCAGGATTTAAAATTCTGCCGGCCCG | |
| ACTTTATAAGGTTCAAGGTAAAGAAAAGAAGCAACTTTAAGAAGAAAAAACGCTTGATTCCC | |
| >‘990820A-007.scf” came from CONTIG 26 at offset 0; | |
| “E:\SEQUENCE\export\EG_DB\990820a\990820A-007.scf’ (58>532) | |
| TGGGACGAGGGGGGGCCTGGGGGCTAAAGGGAGAAAGAGGACGGCTCCTGGGGGGGGAATGGGGGTAGGGATGGCCGCTCCCGGGAGCCCGCGCGACCGCGCC | |
| GGGAGGGTTTTGTGGGCTGCCGGCTCCCCCAGGGCCCCCAAAGGTGTGAAAGGGGGAACGTGGGCAGGTCCTGGTGGGTCCTGGTGGCTGGTGGGTTGCCCGG | |
| TGGGTCGGGGGGCCTCCTGGCCCCTCCTGGGAGGAATGGGTTACCCCACGCCCCCCCAGGCTCCAGGGGGGGCTCCAGGGCAAAGATGGGCCCCCCAAGGTCC | |
| ACCCTGGCAGGTATTGGGGCTTGTTGGGAAGCCCGGGATTCTCTGGACCAAGGGGGTTTTTTGTGACCAGAGGGGAGGGGGGACCCGGGCCCCAGGGGCCTTC | |
| GGGGGCTCAGCCCACTAGAATTGATGATTATTGGAGCCCAAGGTTGAGGCCCCCAGGCTCTAG | |
| >‘990820A-023.scf” came from CONTIG 27 at offset 0; | |
| “E:\SEQUENCE\export\EG_DB\990820a\990820A-023.scf’ (60>543) | |
| GCAGGAGGGTGAGGCGGGGTGGGGGCGGAAGCACGGGTGTCGAGCGGCTCGACTGGGGAGTTTTTGGCTGGAGCAACGGGGAGCACCATTGGATATCTGGACT | |
| TGGTATGACGAGTTTTGGGGAATTACATCGGAACCAGAGCTTGATTCTGATGAAGAATGATGATTGAATTGGGCAGAGAGACCAAAGATCTTGATGAGGTCGA | |
| AGAGGACGAGGACGACGACGAGGGGGCCGAACACAACGAAAAACACCCTGGGTGGTGGGGGGGGTGCAGGAAGACAAAAAGACCACCCCACCGCCGGGGGGGG | |
| GGACGGCCCCAAGGGGGACCATAGTAAGAGGAAGACACCAGCCCTCCAAAACCATATTAAGCAGGAAACCAAAAATTACTCGATGAGCAGAGTCCTGCACGGG | |
| GCGCAATGGTTTTGGCGGAATGATGAAATCGGCTTATAAAATGGACCTTTGGGCTCTGACCCGAAGAAGTTT | |
| >‘990820A-031.scf” came from CONTIG 28 at offset 0; | |
| “E:\SEQUENCE\export\EG_DB\990820a\990820A-031.scf’ (54>351) | |
| CTGCCTGCAGGGGCATCCTGAGTGTGGATTCCACTGTCAGCTCCTCTCCTTCCACCTGTGCTGTGACTCCGAGGGATATGTACGTTGCACTTTCTCCCAGAAA | |
| ATCTGGGCCAAGTGCTGTGAAGCGGGGGAATGGAGAAGGCTAGAGGGAGACCCACTACCACCTTCAAAGACCTGTGCCCAGAGAAACCCAAGGGGGGAAGAAA | |
| AGCTGGCATCGCGGCGNGGGTCTCAGGAGATACCCGATAGAGACTGACAGCTTTGATGTGCCCTGTACCCCAGTGCTTTGCTTACAGGAGGT | |
| >‘990820A-027.scf” came from CONTIG 29 at offset 0; | |
| “E:\SEQUENCE\export\EG_DB\990820a\990820A-027.scf’ (60>518) | |
| GCACGAGGGGGTCCTGGACAGCATAAGCATCATTGACACGCCGGGGTATCCTGTCTGGAGGAGAAGCAGCGGCATCAGCAGAGGGTATGACTTTGCGGCCGTC | |
| CTCGAGTGGTTTGCCGAGCGGGTGGACCGTATCATCCTGCTCTTTGACGCCCACAAGCTGGACATCTCTGACGAGTTCTCAGAGGTCATCAAGGCCCTCAGGA | |
| CCACGAGAACAAGATCCGTGTGGGGCTGAACAAGGCCGACCAGATTGAGACGCAGCAGCTGATGCGGGGCTACGGGCCCTCATGGGGCGCCTGGGAAGATCAT | |
| CAACACCCCGAGGGGTCCGGGTGACATTGGCTCTTTGTCCCACCGCTCTCATCCCGACACGCAGGTTTGAGCTGAGAGAGACTTAAGAATCAGCTTGCCCGAA | |
| GCGCCCTAGAGCTATGACTATAAAGGGCGGGGCCAGGCAGCGATT | |
| >‘990820A-022.scf” came from CONTIG 30 at offset 0; | |
| “E:\SEQUENCE\export\EG_DB\990820a\990820A-022.scf’ (55>247) | |
| TTTAAGTACGAGGGTGAGGTGAGGGGGGGGGAAGCACGCAATCCACCGGCCGACTGTGAGTTTTTGGTTGGAGGAAGGAGAGCACCATGGATACTGAGTTGAA | |
| TGACGAGTTCGGGAATGACATCGCACCAGAGTGGGCCGGTGGAGGAGAGGATGTTGAAAAGGGCTGAGGGACCAAAGATGTTGATGAGGT | |
| >‘990820A-029.scf” came from CONTIG 31 at offset 0; | |
| “E:\SEQUENCE\export\EG_DB\990820a\990820A-029.scf’ (60>526) | |
| GCACGAGGAGCGCCGCTCTGGCCGCACTGCGCTCGCCCTGAGCTCCGGGCTCCTGCTAAGCGAGCGCCGCTGTCGCCTCCCTCCAGTCGCCATCATGATCATC | |
| TACCGGGACCTCATTAGCCATGACGAGATGTTCTCCGACATCTACAAGATCCGGGAGGTCGCGGACGGGCTGGGCTGGGAGGTGGAGGGGGAAGATGGTCAGT | |
| AGGACAGAGGGTAACATCGATGACTCGCTCATTGGTGAAATGCCTCGCTGAAGGCCCGAGGCGAAGGGTACGAAAGCACAGGATCACTGGTGTCGATATTGCA | |
| TGAACATCACTGCAGAAAACAGCTNACAAAGAAGCCACAGAAGACATAAAGATACAGAAGGCAACAAGGAAACTGAAGAAAAGACAAAGAGAAAACTTTTGAA | |
| GGGGCGCAAACAAACAGCCAACTGCTATTAAAACATAAGCTTAGGGGAAAAGAT | |
| >‘990820A-021.scf” came from CONTIG 32 at offset 0; | |
| “E:\SEQUENCE\export\EG_DB\990820a\990820A-021.scf’ (54>537) | |
| CTTAATTCTTCTATGTCCAATGGGAACAAGCGATGCTGGACCCTTTTCGATTCACATGGATGATATGAGATGGACGGAAGGGGNAGGCCGAGGGTCGGTGGGA | |
| AAGAGCACAATGAGTGTTATCATGCCATCTGTTGGTCACAGNGTTAAACTTGGAATGGATCTTACGAAGGTAGTTCTTCCAACATTTATTCTTGAAAGAAGAT | |
| CTCTTTTAGAAATGTATGCAGACTTTTTTGCTCATCCGGCCCTGTFTGTGAGTATTAGGGACCAAAGGATGCCAGGACCGAATGGTTAAGGTGTGAAAATGTA | |
| TTTTTCACCGTTTCACGCAGAAGAAAGGACGGTGGCCAAAAGCCACCAACCCCTTGGGCGAAATTTAGGGCACTGACATACAAAGAACGAGAGAGGGGGCAGG | |
| CCAAAGACAGTCCTGGTTCCAGAAAAGAACATTGGGGCGGCAGTTCCACATCCCCCTTACTTTTTGCGGGGG | |
| >‘990820A-032.scf” came from CONTIG 33 at offset 0; | |
| “E:\SEQUENCE\export\EG_DB\990820a\990820A-032.scf’ (54>526) | |
| TGCCCGTGAACCTGCTCCGAGCCGGCCGCATCTTCGTGTTCGAGCCACCCCCGGGCGTGAAGGCCAAGATGCTGAGGACCTTCAGCAGCATCCCCGTGTCTCG | |
| GATATGCAAGTCTCCCAATGAGCGTGGTCGCITGTACTTCCTGCTGGCCTGGNTCCACGCCGACATCCAGGAGCGCTTACGATACGCACCCCTGGGCTGGGCG | |
| AAGAAGGATGAGTTCGGGGAGTCTGACCTGCGCTCGGCCTGCGACACGGGGGACACGTGGCTGGACGACACAGACAAGGGAGACAGAACATCTCGCCAGACAG | |
| ATGCCCGGTCCGGGCTCAGAGCCTGTGGGCCATGCTNTATGGGGCGGGGGACACGAGTCGACAGCGCTGCTACACTTCCTGACGCCGTCACACCGGGTTNGAA | |
| GGNGGTCAGCTGGCTGCAGGCGAGCACAGGACATCAAGCGGCGCATAGCGAAGGATGGCAT | |
| >‘990820A-028.scf” came from CONTIG 34 at offset 0; | |
| “E:\SEQUENCE\export\EG_DB\990820a\990820A-028.scf’ (60>520) | |
| GCACGAGGGTCATTGCTGGTTCTGGATTTTTGGCACGTCTTTGGGTCTCATGTTGCTAACCCAACACCAGAGGACTTAAGGCTGTCAGGAACAGTTCTTATTT | |
| GGGATTTCAGAGCAATTACTGAAGAAGGATTATGTTCTTGTGAGAAAACGAATTTATGATTCAGAAACCCCTAAAGACAAGGTACTAAGCAACAAAATATGCA | |
| AACAGATGTTGNTAATTAACTGTCATTTTTGTCTTACAGCTGTTTGCAGACAAAGATCCAAAGACAGCAGATTAGTTTTACAAAGGGTATCTGATTATTAGTT | |
| TGTTGTGNGTGTCTCCTTTTGTCCTTTATAATAGTTATATCTTTAATTTACAGAAACTTCGGCTTGACACTGAGAGAAGATTGGTTAAAGTCCTGTTTACAAT | |
| ATTCGGTTTTGGCAGGATGAAAACGTACTCTGGTTGCTATTAGATGACTGN | |
| >‘990820A-034.scf” came from CONTIG 35 at offset 0; | |
| “E:\SEQUENCE\export\EG_DB\990820a\990820A-034.scf’ (60>526) | |
| GCACGAGGGCCGGCTCTGGGTGGTCAGGCCTTGCGAAGCTGACCTGGAGGAGAACATTAAGAAAGGCAAAAAGTGCATCCGGACGCCCAAAATCTCCAAGCCT | |
| ATCAAGTTTGAGCTTTCTGGCTGCACCAGCATGAAGACATACCGAGCTAAATTCTGCGGAGGGTGCACAGACGGGCGGCGCTGCCACCCCCCACAGAACCACC | |
| ACCCTTCCGNGGAGGTTCAAGTGGCCTGATGGGGAGGGCATGAAGAAGAGCATGAGGTTCATCAAGACCGCGCCTGCCATTACACCGCCCCGAGACATGACTC | |
| TTTGGGCCCTGACTACAGAAGATGATGGGGACAGGCCTAAGCCGGGCAGGGGACCGGAACATTTTGCTGGCCTGAATGTTCCATTATTTTGGTGCCGGGGTTC | |
| AGGCCCAGTTTTTAATGGGTTTAAGGGAAAAAAAATCCCCAAATAAAATGGGCTT | |
| >‘990820A-037.scf” came from CONTIG 36 at offset 0; | |
| “E:\SEQUENCE\export\EG_DB\990820a\990820A-037.scf’ (11>408) | |
| AATTCGGGCTCTGTACAACTAGATGATCTACAGGGCTGCTGGAATTATGCACGATTGTCTATTCTATATTGTTCACCCAATATCTTGTGCGTAATAATGGTTG | |
| ATACTTGTTGTCCTGTGGGGAAATATGTTATGCCGGTCTACAATTGCACATCATTGTAACGAAGGCGGAAAACATAAAGATGTAGAAGCGTGGGGAAGGGCTA | |
| AAGGAAGGAGGCTCAAGTGAAATTAATGTGCGATGGGGGTCCCTGCCCCCGCTTTCCCGGGGGCCAAGACCTACCCCGGCCAAGGTGGTTTTAACGAAAGNGG | |
| AACATCGGGGCGAGGAGAGGCGGCGTTTTGCTCCTTTTCGCCTCTTTTGGTTTCTTTTGTTAAATTAAAAAAGTATTGTTGGAAAAAA | |
| >‘990820A-039.scf” came from CONTIG 37 at offset 0; | |
| “E:\SEQUENCE\export\EG_DB\990820a\990820A-039.scf’ (60>580) | |
| GCACGAGGACCCCAGGGCCTGATGGGAAACAAGGGTGGAACCGGGGGGTGGTTGGGCGGCTCCAGGGCACTGTTGGGCCCATTATGGGTCCTTAGCGGGACTC | |
| CCCAGGGAGAGAGGGGGTGCGGGCTGGGCATTCCTGGAAGGCAAGGGGAGAAAAAGGGTGAAAACTGGGCTCAGAGGGTGACATTTGGGAGCCCCTGGTAGAG | |
| AAGGTGGCTCGTGGGGGCTTCTGGTGGCTATTGTGGGCTCCTTGGCCCCTGCTGGAGCCAAAGGGGGACCGGCGGTGAAAACTTGTCCCGCTGGCCCTGCTGG | |
| CCCCTGCTGGTCCTCGGGGTAGCCCCTGGGAACGGTGGGGAGGCCGGGCCCCGCTGGCCCCAACGAATTTGTGGTNCCGGTGGGCTGGTGGTCACCTGTGCTA | |
| AAGAAAAAAAGAACCAAAGACCAAGGGGAAATGGCCTGTGGCCCCCAGCCCCGGGGACCGGCGGCCGCTGCCAAAGCCCCCTGGCCTGCGGAAGCGGGGAGGA | |
| GGGCCC | |
| >‘990820A-040.scf” came from CONTIG 38 at offset 0; | |
| “E:\SEQUENCE\export\EG_DB\990820a\990820A-040.scf’ (55>520) | |
| GGACAAAATCTACTTCATGGCTGGGGCCAGCAGGAAAGAGGCCGAATCTTCTCCCTTTGTTGAGCGACTTCTGAAAAAGGGCTATGAAGTGATTTATCTCACA | |
| GAACCTGTGGACGAATACTGCATTCAGGCTCTTTCCGGTITCGACGGGAAGAGGTTCCAGAATGTTGCCAAAGAAGGAGTGAAGTTGATGAAAGTGAGAAAAG | |
| GAGGAGAGGCGCGAAAGCAGTGAGAAAGAAATTGGGCCTCTGCTAAATGGATGAAAGATAAAGCCCTCAGGACAAGACGGGAAGGCTGGGGGGCTGACCGCCT | |
| GACAGAAGCTCGGGGGCCTGGAGCCGCCGNCGGGGGGTGGGCACTGAGAGGTCAGAAAGCCAGCTCCCGCAGCANGCCTCTCACCATTTTTTGCGGCCAAGAA | |
| AATTGAATTACCCCACCCCCCTATCGGCCGCCGGGGGGAAGAGAGAAAGACAAAG | |
| >‘990820A-041.scf” came from CONTIG 39 at offset 0; | |
| “E:\SEQUENCE\export\EG_DB\990820a\990820A-041.scf’ (60>514) | |
| GCAGGAGGATTTAGTGAGATTTATGCTCTTTAAATTTACATGCGTAGATACAATGTTTATCACTCTAAAATCATTATATAGTTTTATAATCATCTATGTATCT | |
| AGAAAAAATGATTTATTAACTGTAGGATGAAAGACCATTTGGCAGTTTCCCAGACTTTTTGACTTTATCAGATTATAAATAAACTATTACTGCTTACATAATG | |
| GGGGCTTAGTGTTTGCTGTTTTGAGGTCTCATTCTTAAATTTTTCCCCTTCTATAATGGATTATTAAAAGGATATTATATATAATGGATATTATAAAGGGTTC | |
| ATACTAGGCTGAAAAGATCTAGGCAAAATTAAAATATCTTTTTGTGAGAAACTATAATAGTATTATTTGTTTTCACAAAACACTCCATGACTCCTCCTCTTTC | |
| AATTATAATTTAAACCCCCAAAACCCAGACCTTGGCTTGGGGN | |
| >‘990820A-046.scf” came from CONTIG 40 at offset 0; | |
| “E:\SEQUENCE\export\EG_DB\990820a\990820A-046.scf’ (55>533) | |
| TTAAAAAAAAAAAAGGAAGGAAGGAAAGAAGGAGACAGGAAGGAAGTATTGAAAAAAGGATAAAGAGCAGAAAGGAAAGGAGGAACAGAAGGAAAAAGCAAGG | |
| AATAAATAAATCACAGTTACTTGAAATTCATAATCAGAAGAAATGAATTATATCTCCAGCATATGTTGTATAACTCTATGCTAATAAATTTGTAGATCTATTA | |
| GAAAATGTGTGATTAATAAATGATACAAGAAAATACAGAAGATTGGCGTTACTATGAAAGAAATGAAATATTGTTAGTTTTTCTTGAAAGAGGGTGGGGGCCG | |
| CTGATTTTTTAGGGAATTCTTTATGCTTTCTGAACAGATAGCCTGGTTAATTAAATACTTAGATATAAGAAAAAGAAATTGTAGGTATTTTGGGAGGCACTTA | |
| CATGATTAAAACTAAGAACCCCAAGGCTGACCATTAGACAATTTGGGAATCTAATTTAAATTCAACC | |
| >‘990820A-049.scf” came from CONTIG 41 at offset 0; | |
| “E:\SEQUENCE\export\EG_DB\990820a\990820A-049.scf’ (60>510) | |
| GCACGAGGGCGAATCGCAGAGCTCTGCACTTCGTGTTCAAAGGGGAAACCGCTTCGAGACGGCGTGTTTCTATCGCGATGTCCTGGGGATGAAGATTCTTCGG | |
| CATGAGGAATTCCAGGACGGCTGGAAAGCTGCCTGTAATGGGCCTTATGATGGGAAGTGGAGTAAAACAATGGTGGGGTATGGACCTGAGGATGATCACTTTG | |
| TCACAGAGCTGACTTACAATTACGGCATGGCAGCTACCAGCTTGGCATGACTTCCTGGTATCACGGNGGCTCCAGGCAGCTGGCAGCACGCTAGAAGCTGAGG | |
| GGCGCTCAGGAATGGAGATGGGGGGTTGGACCAAGCCCCTGAGATTTTAGTTTTTGCGGACGGGCCCCCTACCAACCGATAAAGAACTTGCGGGGCGACTCAA | |
| AGCCTGACACGGCCTACTACGGGAGAAATTTGAGAGTGG | |
| >‘990820A-052.scf” came from CONTIG 42 at offset 0; | |
| “E:\SEQUENCE\export\EG_DB\990820a\990820A-052.scf’ (57>345) | |
| GGACGAGGGGCGAGGCTATAGGTCCATGGTAATGCTCAAGAAATGTCATGAGATTGACTTTCATTAGACAAAAAACAGCAAGCATTTTTAAACCCTGGTTGGG | |
| CGGTTTTATTTTTCTTGTGAATTTAAAAACTTTTITTAAACTTTTTTTTAAGGGGGGGATGAGAGGGGGACCGGGGGGGTTTTTTTTTTGGCTTTTGGTTTTA | |
| ACCAGGGGGACTTTCGGGCCCAAAAAACATAAATACATTGGGGCACAAGGAGAATAAGGGAAAAAAACATAAACCCTTTGGGGCAAG | |
| >‘990820A-053.scf” came from CONTIG 43 at offset 0; | |
| “E:\SEQUENCE\export\EG_DB\990820a\990820A-053.scf’ (60>202) | |
| GCACGAGGATAATATCGTAGGTTTATAAAACCTATTTATAACACTTTTTACATATATGTACATAGTATTGTTTGCTTTACGTGTTGACCATAAGCCTTGGGTT | |
| GAACCTTAAAGGAGCTAAGGAACTGGAAATCTTAACTTAT | |
| >‘990820A-054.scf” came from CONTIG 44 at offset 0; | |
| “E:\SEQUENCE\export\EG_DB\990820a\990820A-054.scf’ (60>298) | |
| GCACGAGGCCCCCGCCCATGGCTAGCCTCCTGCTCCTGCTTCTCTGCCTTGGTTGGGTGTCTACCTTCCAGGGGCCCATGGCTGCAGACGTGGGAACTGGCTC | |
| TTCTGAGCTCAAGTGTGCAGCGCACATCGCGGGACCTGTCCTGTCAGCAGCCTGAGCTGACCGTCCTCCTCCCGAGGGCCCCGCGGGNCACAGAGAAGAAGCC | |
| CGCCCCCCCGGGCCGAGAGGCCCAAGGGTTGGA | |
| >‘990820A-055.scf” came from CONTIG 45 at offset 0; | |
| “E:\SEQUENCE\export\EG_DB\990820a\990820A-055.scf’ (51>514) | |
| TGGTTTTTTTGGAAAACCAAACATGCTTTATTTCATTTTTTTCACAATTTATTTAAACATCTCACATATACAAAATAGGTACAATTTAATTTTTCTGCTTGTC | |
| CGAGAAACAAGACTTCTTTGGAACCATGGGAGAGGATGAAAATGAGACTGGCAAAGAACAAATGCTGAATTTAAAGAAGAGGACAATGTTGGGCAAATGATCC | |
| ACTTACTTTGGGGGAATAAGAGGAAAGGACTGATGTTAAGACAATGAAAAAAAAAACACAAACAGCTCACAGCGNGGAGGATCTTTCTCAATCCCTAGCAGAT | |
| CAACATTCTCAGAATCTGAAAACTGGTATTAGCACCTGGATTTGACAAAAACAAATACCCACTCTCCTGTCAGACTGCGGGGGACAACCACGAAGGGTTTGGG | |
| GAGTTAAGTTGGAGTGGGTTAACTCCCTCGGGAGAATTAAGCCCGGGGGCA | |
| >‘990820A-056.scf” came from CONTIG 46 at offset 0; | |
| “E:\SEQUENCE\export\EG_DB\990820a\990820A-056.scf’ (54>506) | |
| TGAGAGCCCACAAGGACCACAGTGGAAGATCCTGGCAGAAAGGAGAGTGCCACCCAGCACCCATCCCTCACCTTCCATATTTCCTTTCTCAACACCTGCTAGG | |
| GCCTGGCAAAAGAGCTTTTCCCAGAAGACTTAGGGGAAACCAAACTGATATTTTTTTTGGCCAAGTCATACATCTTGCAGGATCTCATTCCCCAACCAGGGAC | |
| TGAATCCAGCCCACAACAATAAAAGCGCTGAATCCTACCCACTAGACCACCAGGGAAATTCCAAACTGATACCTAGAAGGGGGNGGGTATPATGGAGTTTAGG | |
| CTTCCTGCGCTAAAACACCNCAACCTGCTACTGTATGGGAAAGGNAGGGACCACACAGGAGAAGAACCACCTCAGCCCTGGAATGGGAAGAAAATTACATGTC | |
| AGTTTTGGGCGGAAATCAGGCCAAAAGAAAGAGACCCTC | |
| >‘990820A-057.scf” came from CONTIG 47 at offset 0; | |
| “E:\SEQUENCE\export\EG_DB\990820a\990820A-057.scf’ (54>504) | |
| GCAAGATGATGACTTCTTGGCCACCTTGAAAGAGCTGGAAGCAACCCTCCGGACCCAGAGCCTCTCTCTGGAGTGATTCCTGAAGGGAAGATCCTGAACAACA | |
| CCTACTACCAAGAATGCCTCTTCTAGGTGCACAGCTACAGCAGGCCGCCAAGAGCAGCGGGGACGCAGGGGTGGCAGACATCTGCTCCCAGTGGCTGCTGACA | |
| AGCCGCAGCCGGGGTGCCCATGGTTCGGCCTGCAGCAGTGACCCTGGCTGCAGGCCAGACAGCGNGGGGCAGGAGGATGGCGCCTTTCAGCTCTTTCTCCGGG | |
| GGGCAGGACTTCCTGGCTCACCCAGGTGGAAGGCGGGAAGNACCCACCTGCATCTGAGCAGATGGACTTCCGCAGGCCAGTTTGAAAAGTGAGGCGGGGGGGG | |
| TGGCGGGAGCCGCCTTTACCAGAGGAGGGGGGGGAAGCC | |
| >‘990820A-058.scf” came from CONTIG 48 at offset 0; | |
| “E:\SEQUENCE\export\EG_DB\990820a\990820A-058.scf’ (54>525) | |
| CTTTTCCAAACACGGGAGCCAGGATCATGCTGTTCACTGGGGGACCCCCCACCCAGGGAACCTGGCATGGGGGGTGGTAGATGAAAGGTTCCTATTCGTTCCT | |
| GGCATGACATTGAGAAAGACAATGGCCGCTTCATGAAAAAGGCAACCAAGCACTATGAGATGCTTGCGAACCGCACTGCTGCAAACGGTCACTGCATTGATAT | |
| TTACGCCTGTGCCCTTGATCAGACTGGACTCCTGGAGATGAAGAGTTGTCCAATCGTACTGGAGGGTACATGGGGAGGGAGATTCTTTCAACACTTCTCTCTT | |
| CAGCAGACATTCAAAGATTTTAGTAAGATTTATGGAATTCCGATGGGCTTTGTGTACTTGAAGAAAGACCAGGGGCGGAGGGCGGGAGCATGACCTGGGGGTT | |
| TAAAGGGAGGACGGGGGGCAAAAAGACTGGGGCGGGCAGGGCAAGGAAAATGGGCG | |
| >‘990820A-059.scf” came from CONTIG 49 at offset 0; | |
| “E:\SEQUENCE\export\EG_DB\990820a\990820A-059.scf’ (54>533) | |
| CAGCAGCAGCAGCAGCAGCAGCAGCAGCCTCGGGGGGGGCGGCAGCAGCAGCAGCGGCGGGGCGGCCCGCGCGGGTGTTTATGTGGGGTCGCGGGGTCTCCTG | |
| GCAGCATGGCGGACTACCTGATCAGCGGCGGCACCGGTTACGTGCCCGAGGATGGGCTCACTGCGCAGCAGCTGTTCGCCAACGCCGACGTCCTCACCTACAA | |
| CGACTTCCTGATTCTCCCAGGATTTATAGACTTCACAGCTGATGAAGTGGGATCTGACTTCAGCCCTGACTCGAAGATCACACTAAAGACGCCATCATCTCAT | |
| CCCCCATGGACACTGNGACGGAGGTGACATGGCCTTGCATGGCTTGATGGCGGTATGGGHCATCACACACTGACTCAGAATTCAGCCATGAGGGCGAAGGAAG | |
| AAAATGAGAGGCTCATCAGGCCCGGGGGCTAACCCCGCTACGGGGGAGGGCGAGGCAAACCGGCGGA | |
| >‘990820A-060.scf” came from CONTIG 50 at offset 0; | |
| “E:\SEQUENCE\export\EG_DB\990820a\990820A-060.scf’ (60>529) | |
| GCACGAGGGATCAATGGAACAGATGGAGACACTTTTCACCCGCTAATCACCAAAGATGAAGTCCTTTATGTCTTCCCATCTGATTTTTGCAGGTCAGTGTATA | |
| TAACTTTCAGTGACTTTGAGAGTGCCCAGGGACTGCCTGCCTTGAGGTATAAGGGGCCTGCAGAAATATTAGCCAATACCTCAGACAACGCTGGCTTCTGTAT | |
| ACCTAAAGGAAACTGCCTGGGGTCAGGAGGTTTGAATGTCAGCGGCTGGAAGAATGGCGCAGCTATATCATGCCTTCCCACACTTCTACCAGGCAGATGAAAA | |
| GTTGGCTCTGGCCTGGAGGCATGCATCCAATAGGGATATCATGGGAGCTTGGGGCATCAACCCCTGCTGGATATCCTAGAGCAGCAGAGGTCGAATAATGGTA | |
| TGTAGAAAATAATGACTCATGAACAGAAAATTCAACCCGGTTCCCAGGGGGAATAAAG | |
| >‘990820A-061.scf” came from CONTIG 51 at offset 0; | |
| “E:\SEQUENCE\export\EG_DB\990820a\990820A-061.scf’ (54>521) | |
| TCGGAGTTTGCCACTTATCGGGCATCCTTGGTGTCCGCAGCACTCTCTCCCTCTCTCTAGGGCGGCGACCTCCGGCGGCCGGAAAGTCACCATGTCCATCCTG | |
| AAGGTCCACGCCAGAGAGATCTTTGACTCTCGTGGGAATCCCACCGTTGAGGTTGATCTCTTCACCGCGAAAGGGCTCTTCAGAGCTGCTGGGCCCAGTGGCG | |
| CCNCAACTGGAATCTATGAAGGCCTGGAGCTCCGGGACAATGATAAGACGCGCTACATGGGGGAAGGGGGCTCAAGGCTGGTGAGCACATCAATAAAACTATG | |
| GCGCCGCCCTGTTAGCAGAAGCTGACGCGGGGAGAGAAAGAACGACAGGGAGATAAGAGGAAGGACAAAAAAGGCAGTTGGGCGACGCCTCGGGGGGGCTGCT | |
| GGTGCAGGTGGCGGGGGAGGGGGCCCCCACCCCCACGCCCGGGGGGAGGGGGGAAN | |
| >‘990820A-025.scf” came from CONTIG 52 at offset 0; | |
| “E:\SEQUENCE\export\EG_DB\990820a\990820A-025.scf’ (60>249) | |
| GCACGAGGGTTGGGGTCCGGGCCCCTCGCTTCTACCCTGACCAAAACCGAAGTCCTGACGTGTTGGGGTCCGGCCCCCTCGCTTTTACCAAGACCAAAACTGA | |
| AGTTCTGACGTGTTGGGGGCCGGACCCTCTCTTCTACCCACACTAAAACCGAAGTTCGACGTTTTAGGGGGCCACTGGGCCCGAATC | |
| >‘990820A-066.scf” came from CONTIG 53 at offset 0; | |
| “E:\SEQUENCE\export\EG_DB\990820a\990820A-066.scf’ (55>526) | |
| GCGTAATTTTCAAAAGAAAAACCATAAAGTTAGCTGAATAGTTTCTTTATTTAGACATTGAATCACAACTGTCCACTTAGAAGATTTTTAAAAAAAATTGTTT | |
| ATGTGTCTTTACACTAAAACAGAACTTAAAAGATGAATTTTATGGCTCATGTAACCTACAAGGTTGAGAGCAGYIATAATTTTTACTGGCCTAGCGCCATTAA | |
| AAAGTATTTAAGATTATAGTAGGAAGTATGTAATTATTCATTTATATTACCTTAAACGATGGATGAACCATCACACATTTCAGCCATTAAAAGGAAAATAGAG | |
| CATCATACATGATTCAGAGACAAGCAGCGTATCACTATTAGACTAATAGAATTTTGTTATATTTCCACTTCTGTTTTCAGCGGACAAAGAAAACAGTGGCCGC | |
| CTGCTTGATTAATAACCCCAAACCTTTGCTTTTAGGCGAAATAAACGAATCCAACCA | |
| >‘990820A-073.scf” came from CONTIG 54 at offset 0; | |
| “E:\SEQUENCE\export\EG_DB\990820a\990820A-073.scf’ (60>537) | |
| GCACGAGGCTCATGCCTGTGCTGCTGCAGGGCCAGGCCCGACTGGTGGAAGAGTGTCATGGGCGCCGGGGCAAAGCTGCTGGCCTGCGATGGCAATGAAATTG | |
| ACACCATGTTTGTGGACCGACGAGGGACAGGTGAGCCCCAGGCACAGAAGCTGGTGATCTGCTGTGAGGGGAACGCNAGCTCTATGAGGGGGGCTGCGCCTCC | |
| ACACCTCTGGAAGCTGATATTCAGTCCTGGGGCTGAATCATCCAGGCTTTGCTGGAAGCACGGGGNGGCCGTTCCCACAGAATGAAGCCCACAGCATGGATGT | |
| GGTGGGCCCAGTTGCCATTCACCGCTGGGCTTTAAGCCGAGAATTTATCCTTTTCCTGGCCATGGGGGGTCACGGTCGCGGCCGCCTGTCCTACCGGCTTATG | |
| CCGGACCTGGTGCTTCTTTGTGACCGGGCCCTGCATGAAGAATCCAAAAGTGGGGGCTGGGCCCGN | |
| >‘990820A-064.scf” came from CONTIG 55 at offset 0; | |
| “E:\SEQUENCE\export\EG_DB\990820a\990820A-064.scf’ (60>465) | |
| GCACGAGGTTACAATGCCAGCTTGGGAAAACAGAGAGTGGTTTTCTTCTAAAGGCGATCTGACGAACTCAAAGGTCCTAGAGAAGGAGGTGTCCCGCAGCCCC | |
| ACCACCAGCAGCATCACCAGCGGCTACTTTTCCCACAGTGCCTCCAACGCCACCCTGTCCGACATGGTGGTCCCTTCCAGTGACAGCTCAGATCAGTTGGCCC | |
| TTCCAACGAAGACACAGATTCCAGCGAGCATCCGGACCGTCCCTTGGGCAGATTTCAGACCATCTTCAAACAAGAGTTGACAGAACTAGAAAGAGCTTGGGAA | |
| AGGATAGATGACCGGGTGCCAGTCAGAAAAAGGCCTTACCAAGGGGCCCTTGCCCCCAGTTCCGGGAAAACTCAAACCTCTGTGGACGCTGGTTAGA | |
| >‘990820A-063.scf” came from CONTIG 56 at offset 0; | |
| “E:\SEQUENCE\export\EG_DB\990820a\990820A-063.scf’ (54>534) | |
| CTTTTGTCAATTGTACCTCCATAAAACTGGGAGGAAATGAACAATAAACAGTAAAAGAGTCAGTGTTTTTGTTTTTTAAAGAACAGGGAATCTCAGTGCTACT | |
| CTTACACTGTTTCAGAGAACAGGAAAATAAGGAATACTTTTCATTGTTTTATGAAGCAAGGTTAACACTAGCGCCGAACCTGATGTAAAGATTTACTAAAAAG | |
| AAAAAAAATAAACATAGCTCAGTCTTACTTTTGAGAGCTACAGGGGTAATTTGGCCTTCTAAGAAAAGTTTGTATGTTTTAGATTTTATACTGAAGGATTAAG | |
| GGGAAGATGTCATGATGACTTTTTTTTTTTCTTGCCTGTCGTTTTTACTTTAAATCTTAGATTGAATATATATACCTAGATTTTGGTCTAGGTGGCCGACGCT | |
| TCTTTTTAGGGGTCTGGGAGGAGGAGGCCGCGACCTTTTTGGATATTAATTGAAATGGGGATGGACCGN | |
| >‘990820A-069.scf” came from CONTIG 57 at offset 0; | |
| “E:\SEQUENCE\export\EG_DB\990820a\990820A-069.scf’ (56>464) | |
| AGGCACGAGGGCGGGGGGTGAGGGAGCGACCGGGGGCCGAGTCGCCGGACCCCGGCCGGGGCGATGTGGGAGGCGGGCAGCGGTGGAGCTGGGGGCCCGGGGC | |
| TGTTGGGTATACTGTTGGCCCTCTCGCTGTCGGGGGGCCGTGCCGCCAAGAGCGACGCGGGGCTTGTGACCTGCGTGTCGGTGTTGAAACTTGTTAAAACGCA | |
| GAAACGGGGGCGGGGTGCACTCGCACGAAATAATACGAATCCGCCAAGGCCAAGAGCGGGGGGACGGGGGGAGGGGCCCAAGAACCCCAAAAGTATTGCGGTT | |
| CCCGGGGGGACGGAGGCGGAGGCCCCGCGGTCCCCGGGCCGGGGAAAGGGGGAGCTGTCCACGGCTAACGAAAAACCGCCCCCCCATTCCTGCCGCTT | |
| >‘990820A-071.scf” came from CONTIG 58 at offset 0; | |
| “E:\SEQUENCE\export\EG_DB\990820a\990820A-071.scf’ (56>459) | |
| ACCGTGGCCGGGTGGCCGGGGTACCCGGGGAGCCAACCCCGTCCAGGTGGAAGTAGGAGAATTCGATGATGGTGCTGAGGAAACCGAAGAGGAGGTGGTGGCC | |
| GAGAACCCCTGCGAGAACGACCACTGCAAACACGGGAAGGTGTGCGAAGTGGACGAGAACAACACCCCCATGTGTGTGTGCCAGGACCCCACCAGCTGCCCTG | |
| CCCCCATCGCCGAGAAGAGAAGGTGTGCAGCAACGACAACAGACCTTCGACTCTCCTGTCACTTCTTGCCACCAGTGCACACTGGAGGGCACCAAGAAGGCCA | |
| CAACTCCACTGACTACATGGACTTGAAAACTCCCCCTGCGGGATTCGGCTGCTGAATCCCTTGGCTGGGTTGGAAGAAGGCGGGCGCCGGCGA | |
| >‘990820A-065.scf” came from CONTIG 59 at offset 0; | |
| “E:\SEQUENCE\export\EG_DB\990820a\990820A-065.scf’ (54>525) | |
| CAGATTCCAGGTTGCTGACTCTTTCAGCAGAGCCGGGCCTACGCTTTTGCAACCATGTCTAAAGGACCTGCAGTTGGCATTGATCTTGGCACCACCTATTCTT | |
| GTGTGGGTGTCTTCCAGCACGGAAAGGTGGAAATAATTGCCAATGATCAGGGGAACCGAACCACCCCAAGCTATGTCGCCTTTACTGATACCGCGGTTAATCG | |
| GTGATGCAGCAAAGAACCAAGTCGCAATGAATCCCACCAACACGGGTTTTGATGCCCAACGACTTATGGACGAAGATTTGATGATGCTGTTGCCAGTCTGATT | |
| GAAACATTGGCCCTCATGNGGGNGAAGAGCTGCAGCCTAAGGTCAGNAGAAACAAGGAAGACAAAGCTTTACCANAGAGTGGACCTGGCCTGCAAGAGAAGAA | |
| ACGAAAGCCACTTGGAGACGGTCCAGCTGGGCCAGCCGCTTTTTAGACTAAGGAGG | |
| >‘990820A-070.scf” came from CONTIG 60 at offset 0; | |
| “E:\SEQUENCE\export\EG_DB\990820a\990820A-070.scf’ (54>550) | |
| CGGGGCCGAAGGGGGAGCCTATGGGCTCAGGGGCCCAACTGGGGGATCCCGCCGGCTGGTGCTGGGTGGCCCATGGGCCCTGGGCCGGGAGGACTCGGGGAAC | |
| CTGGCAACGGCGTGAANGCGTTTGACTCGGGTGCTTGGGTGATACGGCNGTCTTGTCATCCGGGCCTGTGCAGTTGGACGTATCCCCTTGCACACCCTACCCC | |
| AAGGAAGAAGGAGAAGTACGCGTGTCAGAGAGGTTGCAGGACTGTTTTCAATTTGTCAGTTTGTGGATGATGGAATTTGATTTAAATCGGACCAAATGGAATG | |
| NGAATCTGCATGTACAGAAGCATATTCCCAATCTGAGAGCAATAGCTTGCCATCTGGTGCCAGACAGCGGCAACCAGAAAGGACAGAGCACTATGGCCGAGCA | |
| AAAGCACTACTTTCCTTTACTTGAAGAAATCTGAGGAAAGGGACCTGACAAAGCTATACCCCTGCGACTTTACTCAGCGTGTGGT | |
| >‘990820A-086.scf” came from CONTIG 61 at offset 0; | |
| “E:\SEQUENCE\export\EG_DB\990820a\990820A-086.scf’ (60>445) | |
| GCACGAGGCTCTGAACTTTTTACGAAGAAATATGGACTTTGTATGGTGAGCTTCATGTTGAAGTGGTTTTTTTTTTTGTTGGTTTTTTGTTTTTTTAATTTGG | |
| AAAATCTGGAAGTTAGCTTGGTTTTAATAGGGGCTATGCTCTACACTCCCCTCCCCTCCCCCCGCCCCGCCCACCCCCCACCCCACTCTTAACGGCCATTAGG | |
| GTTTACTGACCCACTGGCTAAAGTTCCACTCTGTTTACTACATCATAATGCTGGGTTGCGGCCTTTTTGTTTTTTTTATATTATAAAAAAAGAAAAGTGGGGA | |
| TGGGTTGGAGCCCAGCGCCCTGACCTTGGGGGGATGTAACAGGCCCGGGACCGGGGCTCGGGCCCCGACC | |
| >‘990820A-047.scf” came from CONTIG 62 at offset 0; | |
| “E:\SEQUENCE\export\EG_DB\990820a\990820A-047.scf’ (60>514) | |
| GCACGAGGCATGATTAATCACACTGTTCGGGCTGGCCAGTTTTTCATGCATGCAAGCTTGACAATTGAGCACAGTCAGGCGTTTGTATTAAAAACGAAAAAGT | |
| GAAAAAACACAAAACCTACTCAGAGGGTTCTAGTTCAAATTGTTAGTGTAAATTGTAGCCGGTTTACTGAGAAGAGCNGTTAAAAGGCTCACCTCAGGAGCCC | |
| GGTGGGTAACTGGCCAGGCGGAGCGCCCCTGCTGCGGGCGGGCCTCTCTACCGCCCCTGGCGACCGTGACGCGCTGGCGGGGCGCCCGCTGGGTCGGGGGGGG | |
| TGCCGGGTGGCTGGTGGAAGGGGGCAGGTTTCACATTATTGAATTTAAAAAAAATTTTTAGGAGATTTGGGCTTTGTTTAAACCTCGACGGGCTGGGCCGCGG | |
| GGGGGGGGGGGGCCCCACAACCAAACCGGGGAAGAAA | |
| >‘990820A-051.scf” came from CONTIG 63 at offset 0; | |
| “E:\SEQUENCE\export\EG_DB\990820a\990820A-051.scf’ (54>507) | |
| GTTTCGGGGGCCAAGCGGGCTGTTTACTTATGCCGGAAAAGGGAAGTTTAACATCCAGCCCCAACAAAAAGGGCTCCACCAGAAGCGCHGCGTGGCCAGGAAC | |
| CTGGAATGATTGCCGAAGCACAAGAATTACTCCATGCTGCAGCTGATCCGGGCCATTCTGAAAGACCCACAAGATCCCACCAGTGCTTTTGGTGGTTTGGCAA | |
| CCAGACTGAAAAGACATTATTTTACGAGAGGACTTGGGGGACTGCAGGCCCGCCCCCCGCCGTTATGTTGGTTTACCTGGTAACCCCCAAAAGCCCCGGGGAC | |
| GGAACCCGGAACCCTGGCCCTCCTGTTTTTCTGGGCGGGGGTGCAGGGGGGTGCCCGTGACTTTGGGTGGGGACTGGGGGTTGAGGGGCAGCGGCACCTCCCT | |
| GCGCCCGCCCCCGGCCCAGGTTGAGGGGGGTTCTTCCCGCCC | |
| >‘990820A-092.scf” came from CONTIG 64 at offset 0; | |
| “E:\SEQUENCE\export\EG_DB\990820a\990820A-092.scf’ (61>496) | |
| GCACGAGGCTTCTTTCATGGATTGTATATTGTTTTCCTTTATGGCTGATCTCACACTTGCATGCTTCCCTCCCCACCCAAAAGCTATTCTTTAAAATCGGATG | |
| TTTTAAAAGAAAAAAACCCTCAGATTCTCAACAGGAAACAGTAAACTGGTGTGGCAGTAGTTCAGAAATCACGATACAAATGTAAACAAATCACTATGCAAAA | |
| GATAAAAAAGGAACAAAAGGAACAAATCTATGGACAGAAGTAAATGCTAAGAGACCGGAATCATTTTGTTTTCACCACAAATTGCAAGAAAAGAAGAACAAGC | |
| TCGACGGCGAACAAAGAAAAGATAAAGGCCCGACCAGGATAATTAAAAAGAACTGGAGGGGAAGAGGGAAGGGGGGTCCGGGGTACGTGGACCTATCAGGGGC | |
| GGGCACAGGAGC | |
| >‘990820A-093.scf” came from CONTIG 65 at offset 0; | |
| “E:\SEQUENCE\export\EG_DB\990820a\990820A-093.scf’ (55>471) | |
| CGTTGCCGTCGCCATGACCCGCGGTAACCAGCGCGAGCTCGCCCGCCAGAAGAATATGAAAAAGCAGACGACTCGGTTAAGGGAAAGCGCCGAGATGACGGGC | |
| TTTCTGCTGCCGCCCGCAAGCAGAGGTAGCCCCAGGGAGGGGAGGGGAGGCTGGGGTGAGACCTCGGTCTGGTTTCTGAGTGCCCCCGGGTCTGACCTTAAGG | |
| GCAAGGGCGGGAGTTCACATTCAAATGCAGAAGAGGGTAGGACAGCCCGTACTTTGGGCCTCTTGCTGCCATTTGCCCTCCTTCCCCCAACCTTTCCTGGGGG | |
| GGGGGGGGGGATTGGGACAAAACCGTGGCCGGGTGGCCGACTGGACCCTGGCAAAGCAAAGAAACGATGCTTGGGCCACTTTTTCCTGGTTCCGGGGGAACAC | |
| ACCAT | |
| >‘990820A-084.scf” came from CONTIG 66 at offset 0; | |
| “E:\SEQUENCE\export\EG_DB\990820a\990820A-084.scf’ (58>374) | |
| AACACTTTGAAAGAATTTGACCAATAATGTATGTTTGGCTTGTGCTTTAGTTTTGTAAGGCATACTTTTTTGCTTGAATCTGTGTCCAGGAGAGTTTGGATGT | |
| TTAATGGTTCTTGAGCTATATTTAATATATTACCAGTTGAGATATATAAAATTGGACATATTGAAATAAAAACCAGGGGCTAAGGAGGGGGAAAGACGGGGTA | |
| CAGGGAGACCGGAGGGCGGTACCCATATGTGTGGCATGACGAACAACCACCCATTTACCCTCCTGGGGTTCGGTGGAGAAAAAAAAAGAAAACTG | |
| >‘990820A-087.scf” came from CONTIG 67 at offset 0; | |
| “E:\SEQUENCE\export\EG_DB\990820a\990820A-084.scf’ (54>521) | |
| GCTGACTATCTCAACCAACCATAAAGATATTGGTACCCTTTATCTACTATTTGGTGCTTGGGCCGGGATAGGAGGAACAGCTCTAAGCCTTCTAATTCGCGCT | |
| GAATTAGGCCAACCCGGAACTCTGCTCGGAGACGACCAAATCTACAACGTAGGTGGAACCGCACACGCATTTGTAATAATCTTCTTCATAGTAATACCAAGCA | |
| TAATTGAAGATTGGGGAACTGACTTGTTCCCCTAATATTTGGTGCTCCCGATTAGCATTTCCCGAAAATAAAATTTAAGTTTTGGACTCCTCCTTCTTATTCC | |
| TCCTATCCCTGGATTCTTTTTGGGGAGGGGGCGGGAAAGGGGACCGGGGCCCCCCTTTGAGGCACCTAGCCTGGCGGGGTTTAACTTTTTTTTTTATTTTTGG | |
| GGTTTTTATTTGGGGCCTTAATTTTTAAAATTTTAATTAAACCCGGAGGG | |
| >‘990820A-044.scf” came from CONTIG 68 at offset 0; | |
| “E:\SEQUENCE\export\EG_DB\990820a\990820A-044.scf’ (54>605) | |
| GAGACATTCCTCCACCTTAAGGAGGGGGAGCGGGGGCTTCCATTTCCTCCTTCCTCCTCCCTTCCTCCTCGGAGCTCCCCACAGCGCTCCCCGGGCCCCAGGC | |
| ACGCCACCACATGGAAGGAAAGGCCGTTGAGGCATTGTTGTTCCGAGGGATTTTTGGGCGGGGTTCCATGCGGGCGCCCCACTCGGGAGCCCCCCTGGGCCTT | |
| TTCCAGCCACTCCCCACGGGCCCGGGGCTGCGGGGGGGCCGGGCCAACATACAAGAACCCGGGGAGGACGCCCTATGAGTGTTGCTGTGGGACGGGAGCCGAC | |
| TTCTGACCTGGGGGACATCAACGGGCACCACCTCCCAGCAACCACATGGGGGGGGGGGGCCTGGGAAGGCTTGGAGGCGGGGGGGGCCGGCATGAACACCCTG | |
| GGTCAAAGGGCCGGTGGCGGGGGCGGGGCCCCATTTGGGACCCCGGCCGCGGGGGGAGGGAGGCCCCCCTCCGGCCCGGGGCGGCCCCAGGGGAGGGGGGAGA | |
| ATTTGGAGGGGAGAGAATGAGCCGGCTGCCCACCCCA | |
| >‘990820A-026.scf” came from CONTIG 69 at offset 0; | |
| “E:\SEQUENCE\export\EG_DB\990820a\990820A-026.scf’ (60>583) | |
| GGACGAGGGGTACATCTACGAGGATCCAGCTGTCGGGCCATCACGGGGCTCCAGCAGGGCCCGGGCTGGCTCTGGGGGCGCAGCATCGTGCCAGAGGGAAGGC | |
| ACAACGCACCAGGCACTTCAGTGCGCAAAGTCGGGAACCCTCCAACCAAAACCAGATAACTACGGTTCAAGATGGGCGACCTTGGATTGGGGGGTTCACCTTC | |
| GGTCCTCCAAAGTGGGGAAAAAAGACAAGACAAAGGTCGGTGTCCCCCCACCATCTTCTTTCCACTGTGGACCCCTGGGGGAAGGGAGGGGTTCCCCGGGTTC | |
| CAGCCACGAGACTGGCCGACTGCCAGTACCTTTTCTCCGGCCCCACCTGTGCCGGGGGGCCCGCTGGGGGGGGCTTTGACGGGGGATGGGAGGGGTGGACCCC | |
| AGGAGCCATAGGCTCCAGAGGGCACCGGCGGCGGGCAGGGCTCACCCGCGCTGGGGCCCCACCGCCGCCGGCCCCCCGCGCGGCAAAAGGGGCCGGGGAGGGA | |
| GGAGCGGTT | |
| >‘990820A-075.scf” came from CONTIG 70 at offset 0; | |
| “E:\SEQUENCE\export\EG_DB\990820a\990820A-075.scf’ (53>527) | |
| TTTTTGTCTATTTTGTTCGTTGAAGTATCCCAAGCACCTAAAATAATGTACACAGCAGGTGGGTGTATTGAATAAGGGTAATAAATATTTATGAGATACCCAG | |
| TATGTATTGGGACAAATATGAGCATTTAGTTGTGGCATGGAGAATGAGAATGACCTTTGGCAAGGTCAAAGAGAGTGGAAGAAGCAGAGTTTCTCACTTCAGA | |
| ATTTTAAAAATTTACTCAAGGGGAAGCGGGAGGGAAGAAGCCACCGAGGAGGCCTCTTGTAACTGCCTTGATATCGCCAAAGCACAGAAAAAAATTTGGGGGT | |
| TTGCCTGCTCCCTTAATTGTGTTGGCCTAGACTTTATATATCATATGGAACCTATTCAGCAACAACATTTAGCTGGCTGATAAGAGACCTGAGGAGCGAGGTA | |
| CTAACCAAAACGAGCCGGGGGCCGCAAAAAATCTGGATAAGAAAAGGCAACACGAACAGTTC | |
| >‘990820A-074.scf” came from CONTIG 71 at offset 0; | |
| “E:\SEQUENCE\export\EG_DB\990820a\990820A-074.scf’ (60>390) | |
| GCACGAGGCTTTATTTTTGATAThTGTATGAGCTTTTTATAAGTTTTGGATTTTGACCCCTTATCTGTCATACTGTGTGCAAATGTTCTCCCATTCAGTAGGT | |
| TATCTTTTCATTTTGTTGATAGTTTGCTTTACTGAGAAAAAGAGATAATTAGGACCCATTTATTGATTTTATGCTTTTATTTCTTTTGCCTCAGGAGACAGAT | |
| CCAGAAGAATATTGGTGGAATAGAGGGAAAGAGGGGTTTGCTTGGCTTTTTTTTAGAGGTTTATTGCTTAAAACTCGCATTAAGTCTCTATTTGATTTGGGGT | |
| TATTTTGCTTAGAATGAGAAA | |
| >‘990820A-082.scf” came from CONTIG 72 at offset 0; | |
| “E:\SEQUENCE\export\EG_DB\990820a\990820A-082.scf’ (60>513) | |
| GCACGAGGCAGGTTTACAACCTGGTACTGACTACAAGATCCACTTGTACACCTTGAATGACAATGCCCGGAGCTCCCCTGTGGTCATCGATGCCTCCACTGCC | |
| ATTGATGCACCATCCAACCTGCGTTTCCTGGCCACCACACCCAACTCCCTGCTGGTATCATGGCAGCCACCCCGGCCAGGATTACTGTTTACATCATCAAAGA | |
| CCAGAAGCCTGGGTCCCCTCTAGAGAAGGGCCCCTCGCCCCCGCCTGGGGNCAAAGAAGCTTATATAATGGCCGGAAACAGAAACAAAAAACAACCAAGCCAT | |
| GCCCTAAAAACAACGAAAAGGAGCCTTGATGGAGGAAAAAGAAAAGAGCCCCAGCGGGACCCTCCACCCAACTTATGGCAAAATTGGTGGTCCTCCAAGAAAA | |
| AACCCTTTTACCAACCGGTTGAACGAAAGTTTAATTCCGGCT | |
| >‘990820A-076.scf” came from CONTIG 73 at offset 0; | |
| “E:\SEQUENCE\export\EG_DB\990820a\990820A-076.scf’ (54>517) | |
| TTTGTATAGAGCTGATGTGTGGGCTTTTAGAAACATAAATAAATAATAAAAGATCAAAAAGCAGGAGGCACATGACTATACTGGAAAATATAAAAGCCACATC | |
| TGTTTCCATAGTTGCCTTTTGACAGCGATTGGCAAAGAACCATTGAATGTATTCTCACAGTCTTTGCTTATGTCATGGAACCTTTGTCCCTAACTCTCCTTTC | |
| CTGAGGTATCGGGGCAGAAGGGTTGAATTATGAAATTGGGGTCTGTAAGGATTTGTTCAGCTTGAGTTGTTGCTCTTTAGTTGGTTTCCTGTGCTTTCATGGG | |
| ACGGTGAGCTCTGTTAGAAGATTTCAAGCCTCTAACCACCTATTATGGTGGCAGCGAGGAGCGCTGGACTGCGCAGACCAGAGGCCGTGCTTGGTGCTACACT | |
| TGGTCATATCCTAATTAATGGCCTCATGGGACGTTTATTTATTAATGTGTT | |
| >‘990820A-080.scf” came from CONTIG 74 at offset 0; | |
| “E:\SEQUENCE\export\EG_DB\990820a\990820A-080.scf’ (54>517) | |
| CACAGCCTCCTTTCAGCTGTTTCTGTCTCAAAGTCTTTGCTAAAACATTTCTCTCTGCTACGATCCTCTCCCATTCTGCCCCCAACTTCATCTCATTGTTGCT | |
| GTCATGTATTAGTCCAAATGTTACTTCCCAAGGAAAGCTTTCTGACAGGACCAAAATAAGTCTGCATTTCATATGCTTCAGAGCCACCTGAACTCTAAGCTAT | |
| TCACTATTTTATGTATTTAGGTAATTTAGTGATTGAGGTGTGTCTCTCTTATGGACTTTAAAGTCTAGAGAACATAAGCCCTGGCTCTTAGCCTCTCTGATCT | |
| CAGAGCCCAGACAAAGATGAAGAAGAACCACTCATCCGGCATCAGAAAGGGAAATTCCCCAGCTCCAGCCCAGATGGGCTTCTGCCAGCAGGGCCGGGATCCG | |
| GGTAAGCCGGCCGATAGGCGGGAATTAGCCGAGCTTTGGGGGAGCGGTT | |
| >‘990820A-78.scf” came from CONTIG 75 at offset 0; | |
| “E:\SEQUENCE\export\EG_DB\990820a\990820A-078.scf’ (60>424) | |
| GCACGAGGGTGCTCCCGGCAGCCGCGGTCCTCTCAGTGCATGGGGCGCCGCTCTGGTTCCGGCCCTTGCTGGTGCGGGAGGGGGGTGCTGGCGCGGGAGCGCT | |
| TTTTCNGTTTTGGCTGGCTGGCGGGGACTTCGGGGAAAGGGAGGGGGAATGCGGGAGCAGAGGGAAGGCACCGCCCCCCCCATGGGGAGGGCTGGTGGCCTCC | |
| GGGGCGGGAGATTCAGGGGCCGGGGGGCCCGCGGCGGGACCGGAGCCCCCGGGACCCTTTCTGCCCAAACCATGAGCATTCTTCAAAGAAAGCAATGGGTTGT | |
| TTGGTTTTTTGAGACCCAAAGGCCCCAGGGACCCTGCCCGGGAGGCAGCATGGGGG | |
| >‘990820A-077.scf” came from CONTIG 76 at offset 0; | |
| “E:\SEQUENCE\export\EG_DB\990820a\990820A-077.scf’ (54>528) | |
| TAGTAACTATTGGCATTAACCAACCTTACCTAGCTTTCCTCCACATCTGTACCCACGCCTTTTTCAAAGCTATACTAATCATATGCTCCGGTTCCATTATTCA | |
| CAGCCTAAACGACGAACAAGATATTCGAAAAATAGGAGGCCTATTTAAAGCCATGCCATTCACCACAACAGCCCTCATTGTTGGCAGTCTCGCACTAACAGGA | |
| ATACCCTTCCTCACAGGATTCTACTCCAAAGACCTAATCATCGAAGCCGGCAACACGTCTTATACCACGCCTGAGCCCTTCTTATAACATTATTGCCACTCTT | |
| TTAAGGTATTTACGCACCGTATTATTTTTTGGACTTCTGGACACCCGATTCCTACCTAGTAAATTAAGAAACACCCGTCTGATCAACCAACTTCGCTACTATT | |
| GGAGCCGTTCCAGATTCATATTCCACATATCCTCACACAATCCCAATACATGCCTATCCTAAC | |
| >‘990820A-081.scf” came from CONTIG 77 at offset 0; | |
| “E:\SEQUENCE\export\EG_DB\990820a\990820A-081.scf’ (54>539) | |
| GGTCTGCGGCTCCAAGTATCTTGGGGCGGGGCGAGGAGTGCCCTCGACCACGTGTGGATCTGCTCGTGCTCTTTCCCGNCCATTGGGCCTCCTGTGTGACGTG | |
| GGTTGACATGGGGCACACCTGTGGGGCCTGGGGTGCTGACCTCTGGCTACCCCGCACCTGACCAACCAGATGTGGGGGAAGGAATTTAGGGCTGTTTCTCTAG | |
| CCCCCACGGAGCCACCAGNGAGGGGGGTCCTGCCCCAGACCTCTGGACCAAGATTATACAGGGGAAATGAAAGAGCCCGAGCACTCTGCCCAGAACCTTGTCA | |
| CAAGAATGCCAAAACGCGCATCGGCCACGCAGGCAACAGGCAGCCTGGGAACCAAGCTGGAAACATTCTTGCCCTGGGTCTGAGTGGACCCATGNAGCATCTG | |
| GCTATTGAAGAAGAAAAAAAAAGGGGCGCAGGCCCTTGCTTTGAAAGCCACCCTTTACTTTAAAGGCTAGGTTC | |
| >‘990820A-090.scf” came from CONTIG 78 at offset 0; | |
| “E:\SEQUENCE\export\EG_DB\990820a\990820A-090.scf’ (60>330) | |
| GGACGAGGTGAGAGCCCTGCAGGTGCGGACCGGGAAGTTCAGGCCCAGCGATGAGCACCACCCGGGAAGTCAAGGCTGATGGATACGTGGACAACCTCGCAGA | |
| GGCCGNGGACCTGCTGCTGCAGCACGCGGACAAGTGACGGGCCTTCTGGGAGGGCCCCGCCTCCTGCCACCCTGCCCCGTGCCCCAACCACCCAGGGTGCTGC | |
| CCTCGGCCCCTGGATAAGAAGGGAGAAGGGGGCAGCCAGGCTGGTTTGGGCCCCCGGGCCACCCA | |
| >‘990820A-088.scf” came from CONTIG 79 at offset 0; | |
| “E:\SEQUENCE\export\EG_DB\990820a\990820A-088.scf’ (54>514) | |
| CTCGCGGCGGCAAGATGGCCGTGCAAATTTCCAAAAAGAGGAAGTTTGTCGCTGATGGACATCTTCAAAGCTGGAACTGTAACGAGTTTCTCACTCGGGAGCT | |
| GGCTGAAGATGGGTACTCTGGAGTTGAGGTCCGAGTTACACCAACCAGGACAGAAATCATTATCTTGGCCACCAGGACACAGAGGACTTGGGGAGGGGCCGGG | |
| GGATCCCGGGATTGACTGCTGTGGTTCAAAGAGTTTGCCTCCCCTGAAGCAGGGTAGAGCTTTATGCCGAAAAGGAAGCCCCAGAGGCCTGGGGCCCTTGGCC | |
| AGCCGGGTTCCGCGTTCCAACTCCAGGGGCCCGCGGGCGGGGCCCGCTTGGGGCCGGGGCCTAGGGGAGGGGCCAAGCGGGAGGCGGGGGCGGAAAACCGGGC | |
| AAGGCCAACCCGAATGGGGGGCCCGAACCCCGGGGCCCCGTACC | |
| >‘990820A-085.scf” came from CONTIG 80 at offset 0; | |
| “E:\SEQUENCE\export\EG_DB\990820a\990820A-085.scf’ (54>520) | |
| GTTGAGAGGAGCGTGGCCTTCTCCTCTCCCGCCATGGCGTGTGCTCGTCCACTGATATCAGTGTACTCCGAAAGGGGGAGTCCTCTGGCAAAAATGTCACTTT | |
| GCCTGCTGTGTTCAAGGCTCCCATTCGACCCGATATTGTTAACTTTGTTCACACCAACTTGCGCAAAAACAACAGACAGCCCCTAGCTGTCAGTGAATAAGCA | |
| GGCCATCAAACCAGTGCTGAGTCTTGGGGGACCGGCGAGCTGTGGCTCGATTCCCAGGGTTCGGGGGGGGGGGACCACCGTCCCGCCCGGGGGCTTTGGAAAC | |
| AGGGGGGGGGGCCGATGTTGGGCCACTAAACCTGCGCGGTGCCCCGAAAGGAAACCACCAAACCAACCCCTCCGCTPTCCTGCTGCCCACCCTCCCCCCCGGC | |
| AGGCAAAGGAAAGAAAAAGAAAACCCGACCCCTTGGGGGGGGAAAAGGAGGCCCCC | |
| >‘990820A-067.scf” came from CONTIG 81 at offset 0; | |
| “E:\SEQUENCE\export\EG_DB\990820a\990820A-067.scf’ (54>455) | |
| GTCAAATTGTGTCCTCCATCACTGTTTCCCTGAGGTTTGATGGTGCCCTGAATGTGGATCTGACAGAGTTCCAGACCAACCTGGTGCCCTATCCCCGCATCCA | |
| CTTCCCTCTGGCCACATACGCCCCTGTCATCTCTGCTGAGAAAGCCTACCATGAACAGCTTTCTGTAGCAGAGATCACCAATGCTTGCTTTGAGCCAGCCAAC | |
| CAGATGGGGAAATGTGACCCTCGCCATGGGAAATACATGGCCTGCTGCCTGTTGGACCGGGGGATGGGGTTCCCAAAGATGGCAATGCTGCCATTGCCACCAT | |
| CAAGACCACCCACCATCCAATTTGGGGACCGGGCCCACGGCTTAAGGGTGGATTACTCCACCTCCCCTGGGACCGGGGGGAACGGCAAGGACA | |
| >‘990820A-068.scf” came from CONTIG 82 at offset 0; | |
| “E:\SEQUENCE\export\EG_DB\990820a\990820A-068.scf’ (58>322) | |
| AGTGGCGAGGTGCGTCCTTGTGTGCCTGGCCTCTFITGCTGGTCCTGCCTTCACGGGGTGTGTCGCCAGTAGACCGCGGGAACAAGGCTCGGTCCCGCTAGCC | |
| ACCGCCGCCTACTCTTGGTCTTACAGATCACCGGCCTGGACTTCCGTGGGTGCGCAGAGGCTGCCATGTCACCGGGTTTAGGATCACCCCTCACCAGCCGCAT | |
| CGCGATGAGCCTGGAGAAGGGGGGGTGCTGACGCTGACCAGAGGCGCGAAAGATAAAGAG | |
| >‘990820A-095.scf” came from CONTIG 83 at offset 0; | |
| “E:\SEQUENCE\export\EG_DB\990820a\990820A-095.scf’ (1>528) | |
| TGTTCTCTCATCCGGGTGTGTGTCGGGGCTGCTTCTAGTTCGTTGTTGGCATCCCGCCTCGGTTTCGTGCTACTGATGAGGGCGTGTGACGCCACCTGTGTCA | |
| TCCACTGGCGTCGTGTTCGGCTCCTTTGTGGCCTACTTTGCTTTCTCTGCCCGGACTGGGTGGCGGGTCCCCCTGGGTGGCCTGGAATACGTGCTTTTTGGGG | |
| TTTGCACTCTCGGCTCTAACACGCTTTGCAACTATTTCTCTTCTCATTCCTATAGTCCTTCACTCATGGCTTGGTTTCTTTTGCGATGTCCAACAGCCAACCC | |
| CGCCCCCACGTCCCCCTCCACTCTCCCTCCTTCCACACATAATATAATAAACATTGTAACTACAAATTGTAAGTGAAAGTCTCACTGTTCTATTAATGTTGCG | |
| TGCTTTTCTCTAACCCTCTTATCCTTGCTTGTCTCATTTTGGCTTCTCTTTATTCTTCCTCAATCAATTCACATAAATATCTTATTTATGTTG7TTTGTTCTC | |
| TTGCGTTC | |
| >‘990820A-091.scf” came from CONTIG 84 at offset 0; | |
| “E:\SEQUENCE\export\EG_DB\990820a\990820A-091.scf’ (56>478) | |
| GCTCTGAGTGGTTTCCTGTTGCCAGGGCTCTAAGACCCCTCACATTCCTGCAGACCTCCAAACTGCCCGGGGCTTGCCTGCTGCCTGCCTGCCTGCCACTGAG | |
| GGTTCCCAGCACCATGAGGGCCTGGATCTTCTTTCTCCTTTGCCTGGCCGGGAGGGCCTTGGCAGCCCCTCAACAGGAAGCCTTGCCTGATGAGACAGAAAGG | |
| GAGGAAGAAACCGGGGGCGAGGGGGCCGGGGACCCGGGGGAGCCCACCCCGCCCGGGGGAAGTAGGGAAATCGGGGGTGGGGGCCGGGAAACCGGGAGGAGGG | |
| GGGGGCCGGACCCCCGCCAACCACAAGCAAAACGGGAGGGGGGGAAGGGAGAAACAAACCCCTGGGGGGCCCGCCCCCCCCTGCCTGCCCCTGGGGAGTGGAA | |
| GGGGCACACAA | |
| >‘990820A-082.scf” came from CONTIG 85 at offset 0; | |
| “E:\SEQUENCE\export\EG_DB\990820a\990820A-083.scf’ (58>513) | |
| GGGCACGAGGCCCAGCAGGGCCCTAGCCTTGCCCTGCCGTCCTCCCATCACTCTAGCCCACCTGTATCAAGGGCCGGGGAGGGGCCGCCCCTGCTGCCCACCC | |
| CAGCATGAGGTTCTGAGCCACACCCTCCCCACAGACAGCCGCTCCGACCGCTGGTCCCAGATGCCAGCAAGCAGGGGGTGAAGGAGGACACAGAGGACGGGCG | |
| GCCGAGGCCCGGGACAGCCCAGCCCCGGTCCTTTCGCATCCCTGCCCACCTCAAAGCACCGGGGTTATGCAAGACGCGGATGAGAGGCACCGAGAAACAGCCC | |
| GGGGCCCAGGACGAAGCCCCACCCCAACCCAACAAACCAGAACCCGGGCTGGGGAAAAATGAATTGGAAAGGGGGAAACACAAGAAGGGGGGGGGCCCCCCAG | |
| GGCCCGGCCTCCCCCGGCCGGGGGGGCCGGGACCCATGGCCCTT | |
| >‘990820A-089.scf” came from CONTIG 86 at offset 0; | |
| “E:\SEQUENCE\export\EG_DB\990820a\990820A-089.scf’ (46>647) | |
| CGCTTGCAGGGCAGGCAGCGGGAGCGCACGGGAATGTTTGTGCCTGGCGTTGGCTGCGTTACCTTCAGGGCGGCCGGGCGCGAGAACCCTGGACCCATGGGAA | |
| ACCCTCACAGGCTTTCCGTTGGGGCCCCTTTTGCAGCTTGGCGCTTTTTTGGCCCCTAACGCTTTTCAAACGGGAGGGTTACGGTTATTGGGCAAATTTGGGT | |
| TGGGAGAAAGATTTTTTTTAATTTGGTTGCCTTTCTGAACAACTTTGAATAAAGTGGAAGGGGGTTTCTCAAAATTCAAACCACGGGAACAAGGCCCCCAAAA | |
| TGTTTCCAAAATTTAAACAACCAAAAACAATGGATTTGGGTGGTAGGGGGGTTTTTATGGGGGAATTTGGCTTTCCCCTTGCCCAAAAATTTTTTGAAAGCAA | |
| GAACTAAAAAAGTGCCCAGCCTTTCCACACATAAGGGCCTGACCCCCGGGGGCATTTGGTGGGTTGTTTGCCCCAATTCCTGGCCTCCTGGCCTTTTCCTTTT | |
| TTTTGTTCCAATATTTGGGGGGGTTTGGGATAAGGAGGGGCCGGTTTCCCGTTTGCCCCGTGGGCCCCCCCAAAAAATTTAGACTTTT | |
| >‘990913a2-001.scf” came from CONTIG 1 at offset 0; | |
| “E:\SEQUENCE\export\EG_DB\990913a2\990913a2-001.scf’ (57>62) | |
| TTAGGG | |
| >‘990913a2-002.scf” came from CONTIG 2 at offset 0; | |
| “E:\SEQUENCE\export\EG_DB\990913a2\990913a2-002.scf’ (13>49) | |
| GTCTGAATAGGGATCCCCGGCTGCTTTTTTNGTTTGA | |
| >‘990913a2-004.scf” came from CONTIG 3 at offset 0; | |
| “E:\SEQUENCE\export\EG_DB\990913a2\990913a2-004.scf’ (51>218) | |
| CTTTTTCGGCTGGAGCGGGGCCCGTGCGGGATCGTGGTGCTTGGGGGTGAGGGGCGAGAGGGTGCGGGGGTGGGTCGGCGGATTGGCTGGGAGGGAGGATGCC | |
| CGACGAACGGTGGCCGCTTGCGGGGGCCCATTTTATGTGGTAAGGGGGGTAGAAAGGGATGGGCG | |
| >‘990913a2-005.scf” came from CONTIG 4 at offset 0; | |
| “E:\SEQUENCE\export\EG_DB\990913a2\990913a2-005.scf’ (51>356) | |
| CCAAAATCTACAGCCGCTCACTGTTACCTGTCCTGCGTTCAGGACGTGTTAAGGCCGTTGCACATATTACTGGTGGAGGAAAACTGGAAAACATCCCCAGAGT | |
| CCTCCCTCAGAAATTGGGGGTGAATTTAGATGCCCAGACCTGGAGGGTCCCCAGGATCTTCTCATGGTTACAGCAGGAAGGCCACCTCTCTGAAGAGGAGATG | |
| GCCAGAACATTTAACTGTGGGATTGGGGCTGCCCTCGGGGTATCAGAGGACCCGGNGAAGCAGACTCTGCGGATATTGAGCAGCACCAGAAGAAGCCGCG | |
| >‘990913a2-007.scf” came from CONTIG 5 at offset 0; | |
| “E:\SEQUENCE\export\EG_DB\990913a2\990913a2-007.scf’ (57>326) | |
| GCACGAGGCACGTGTGATTGTGTCTCCACACATCCGTGCATGTFCCCTGCCTTCCCCTCCGCTCCCTGCCCGCCTGCCCTCTGGCCCTTACCATGGGCGGGCC | |
| CCTGCAGTGTGGTCTGTTGCCCAGGAGGCGAGCGCAGGGACTGAGCTAGAGGATACAGGAGCCTGGGCTCCCAAAATGCCAAAAACTCACACATATTCTCGCT | |
| GAAGGGCCGTAGCCTGCCTCACCACCATTTCACACCCTCACCTGGGACCTGGGCTCTTTTTTTT | |
| >‘990913a2-008.scf” came from CONTIG 6 at offset 0; | |
| “E:\SEQUENCE\export\EG_DB\990913a2\990913a2-008.scf’ (49>556) | |
| TGTTAGCTTAAAAGATGGCGGGTTCCGCTGTTTGCCGGGCAGCTGGCGCCGGGAGCGAGTGCTGCTACGCACCCGCCGCTCGCCGGCCCTGCTGAGTCGGCTG | |
| ACTGGGGGGCACCGCCACCTACGCCCAGGCTCTCCACAGCGAGCCAGAGACGCAAGTCAGCCAGCTGGACAACGGGCTGCGAGTGGCCTCGGAGCAGTCTTCC | |
| CAGCCTACCTGCACGGTGGGGGTATGGATTGATGCTGGCAGCCGNTACGAGAGTGAGAAGAACAAGGGGGCTGCTACTTTGGGAGCATCTGCTTTCAAGGAAC | |
| AAACATCGCCTGGCATGCTTGGAGAGGAGGTGAGAGCATGGGGCCATCTATGCCTACACACCGGAGCCACGCTTATAATAAGAGTATCAAGACTGCAAAGTGA | |
| GAGTCTGCCGCACTGCAGATCANGTCAGACTCGAATGAGAGAGCGGAGGATCTGAGAGTGAGAATACCATCTGCGACGTCTTATACTGTTGCCGCT | |
| >‘990913a2-009.scf” came from CONTIG 7 at offset 0; | |
| “E:\SEQUENCE\export\EG_DB\990913a2\990913a2-009.scf’ (51>340) | |
| TTGTTCCAAGATTATTTCAGAGCAAATCTGAAGGAGGACCACTGACAAGCCCAAAATATTTAATATACCTGATGAAATGGCCAATCAATATATGGAGAGGGCC | |
| ATCAATGTAACCTCCCTCAAAATTTAAACCACGTTAGGTATGACCTACACATTAGCTATCACCTATGTCACACTGTACTTAAGCTTACTCCCAAGTGGAAGCT | |
| ATTTTTATATTTTAGANTCAGTCGCTCAGTCATGTGTGACTCTGCGACCCCAGGACTGCAGNACTACAGGCTTTCCTGTCCATCA | |
| >‘990913a2-010.scf” came from CONTIG 8 at offset 0; | |
| “E:\SEQUENCE\export\EG_DB\990913a2\990913a2-010.scf’ (47>342) | |
| GGTTTTTTCACCACATCAATGGGGTAACGGTTTTTCCCAGCCACACGCTGCATGACAGGCCACCGGCCATTTTTCAGTTGCTGAATAAACGCGCCGGGAATAC | |
| GACGGCTACCCACCACAAGCACGCTGCCGCCACCTTTCAGGGATGAACGCTGCCCCTTTTTACGACGCCTGCGGCGCGAAAGGAGAACCCGCGCATTACCCAG | |
| CTTGATTACGGGCAAATCCCCCCGGTTAACTTTGATTCTGGCCTGCGGATTTTTGACCGTGGGCCTTTTCAGCCTGGCCCTTTCTTTACC | |
| >‘990913a2-011.scf” came from CONTIG 9 at offset 0; | |
| “E:\SEQUENCE\export\EG_DB\990913a2\990913a2-011.scf’ (62>109) | |
| AAGCAACGGCACGGCCAAGGGCATCAGCTGCCACTTCTGGGGACGTGG | |
| >‘990913a2-014.scf” came from CONTIG 10 at offset 0; | |
| “E:\SEQUENCE\export\EG_DB\990913a2\990913a2-014.scf’ (52>227) | |
| TGGCACGAGGCAGAGGCTCCAGGAGGCCCTGCAGGTGGAGGTGAAAGCTGGGAGGACGAGGAGGCCGTGCGCCTCGCCCAGACCAGACTGGTAGAGGAGGAGG | |
| AGGAGAAGCTGAAGCAGCTGTTGCAGCTGAAGGAAGGAACAGAGAGGCGCTTACATCGAAACGGGCACAGGCA | |
| >‘990913a2-015.scf” came from CONTIG 11 at offset 0; | |
| “E:\SEQUENCE\export\EG_DB\990913a2\990913a2-015.scf’ (56>572) | |
| GCACGAGGGACAAAATAGCCCTTCTCCCTATGGAGAACCAAGCAGTTTCCATAACAGAGCCACTTGCTGGATGTATTCTCGAAGGACTCAAGCAAGCGTACAC | |
| TAAGACTTTAAACTATGCTAAGTTGAGTGACATATAACAGGGAGAGAAGGAAACTCCTGATAAACCTCTAGTATAGACTACGGGAGGCTCTCTGCAAGTTTAC | |
| TGACATTGATTCTAAAAGGGCAGACAGAGAAATTTTCTTAAAAGATAGATTTCTCACTCAGACAGCTCCAAATATCTGCCATAAGATACAAAACAGGCATTGG | |
| ACCAATCAGACTTTAGAAAACTGTGCAGCTGGTCAGTGGTGTATATTGTAGAGATATGAGAGAAAATAGANGCATAAAGAACCAGCAAGACTGAGCCCACTGG | |
| CTTAGATTGCTTGAACAACTGGAAAAGCCAGGNAACGGNGAAAGGATGAGCTGTTTTGGGAAGAGGATGTAGTGGAGCTTAGCTCTACTACCCGCCCTGTAGC | |
| T | |
| >‘990913a2-017.scf” came from CONTIG 12 at offset 0; | |
| “E:\SEQUENCE\export\EG_DB\990913a2\990913a2-017.scf’ (49>312) | |
| TATTTGGCACGAGGGTCCCTCATTCCTACTACTCCTCGCATTCTCTATATTTGAAGGTGGGGGAGGGACAAGGTGGACCGCGGTCCCTCCCTTTGCAAGGGAA | |
| ACTAAGCCATGCCGGGGGCTTGAGTAGATCTATCCATTTTCTCTTTTCACTTTGTAGGAGTTTTGTCAATTTTTGGAGCCATCAACTTCATTTCAACAATTAT | |
| CAACATAAAGCCCGCCGCCATGGGACAACACCCAACCCCCTGTTCGGTGATCCGCGTG | |
| >‘990913a2-016.scf” came from CONTIG 12 at offset 6; | |
| “E:\SEQUENCE\export\EG_DB\990913a2\990913a2-016.scf’ (55>576) | |
| GCACGAGGCTCCCTCATTCCTACTACTCCTCGCATCCTCTATAGTTGAAGCTGGGGACAGGAACAGGCTGAACCGCGTTCCCTCCCTTAGCAGGCAACCTAGC | |
| CCATGCAGGAGCTTCAGTAGATCTAACCATTTTCTCTTTACACTTAGCAGGAGTTTCCTCAATTNTAGGAGCCATCAACTTCATTACAACAATTATCAACATA | |
| AAGCCCCCCGCAATGTCACACTACCAAACCCCTCTGTTCGTATGATTCCGTCATAATTACCNGCCGCACTACTACTACTCTCGCTCCCTGTNTTAGCAGCCGG | |
| CGTCACAATGCTATTACAGACCGGAACCCTAATACACCTTCTCGACCGGCAGAGGNAGAGACCTTTTTATTTAACACTATCTGTTTCTTGGCACCGCAGTCTT | |
| ATTTTATCTACTGNGTGGGATAATTTCTTNGTGACCACTATAGAAAAAAGAACATCGATTATGGGATGTGGGCTTATGTAATCGTTTCTAGTTTGTTGACCAC | |
| CTTTATA | |
| >‘990913a2-019.scf” came from CONTIG 13 at offset 0; | |
| “E:\SEQUENCE\export\EG_DB\990913a2\990913a2-019.scf’ (48>315) | |
| CAAAATCGAGAATAATGAGGGAGTTCGGAGGTTTGACGAGATCCTGGAAGCCAGTGATGGGATCATGGTGGGGTCGTGGTGGATGTGGGCATTGAGATACCCT | |
| GGAGAGAAGGTCTTCCTFGCCCCAGAGATGATGATTGGTCGGNGCAACCGAGCTGGGAAGCCCGTCATCTGTGCCACACAAATGCTGGAGAGATGATCAAGAA | |
| GCCTCGCCCTACCCGGCGGAGGGAGNNGACGGGCCATGCCGGCTTGATGGACGGACTGCTCA | |
| >‘990913a2-020.scf” came from CONTIG 14 at offset 0; | |
| “E:\SEQUENCE\export\EG_DB\990913a2\990913a2-020.scf’ (48>580) | |
| CGAGTGGGAGCCCCAGGGCCGCGGTCTGGAAGCGGAACTTGTCGGATTGTTTTAAGAAAATGGCAGACAAGCCCGACTTGGGGNGAAATCAACAGCTTCGATA | |
| AGGGCCAAGCTGAAGAAGACTGAGACGCAAGAGAAGAACACCCTGCCGACCAAAGAGACCATTGAGCAGGAGAAGCAAGCAAAGTGAGATTTCCCGCGAACCT | |
| GGGGATTCTCCACCGCATCATGTTGGAGACCCTAGCCGNGGTGTGGAGGGAGAGCCACCTGCANGATGTACACGAGCGACAGCTGGACTGTGAACCCGGGCNC | |
| TCGTGCCGGCGCACCGGCCGCGGGGGTTGATGGGACCTTCCATCGGACTGCCAATTTTCCGGTTGGCTGGATATTATAAATATTTGTTGATAATAAAATAACA | |
| CACCCGGGAAAAAAAAAAAAAAAATGGGGGGGCCGGCCCATTCGCCTTTGGGGGTTTTCATCACTGGCGGTTTCACGGGATGGGAACCCCGGTCCACTTATGC | |
| TTCCCCTCCCTTTCCCTT | |
| >‘990913a2-021.scf” came from CONTIG 15 at offset 0; | “E:\SEQUENCE\export\EG_DB\990913a2\990913a2- |

021.scf’ (45>59)
TTTTTTTTTGTACGC
>‘990913a2-022.scf” came from CONTIG 16 at offset 0;
“E:\SEQUENCE\export\EG_DB\990913a2\990913a2-022.scf’ (53>59)
GTGACGA
>‘990913a2-025.scf” came from CONTIG 17 at offset 0;
“E:\SEQUENCE\export\EG_DB\990913a2\990913a2-025.scf’ (47>348)
TGTTTTTTCTTCCTTGTGCAGAGATGCTATCACCCACACATCTCCCCACCCTGCCACATGCAGCCTGGAATTCTCCTATGGTTTTCCAGTGGGTCTGTAGTCT
GCCAGAGGAGCAGGGGGTGGCCTCAGACAGAAGCCATGAAGAAGATCCCCACACAAAGACTTTAAGATCCACAACGCTTACTTGTTTTCATTTATTTTGATAG
AGCCATACAAATCANTATTCCAAAGGATGGGGAACAGCGCAGCTAAGCAGACTTTATTTCCCAGCCTATCATATGCTCGTTGNTTCTGCCTTAGTA
>‘990913a2-026.scf” came from CONTIG 18 at offset 0;
“E:\SEQUENCE\export\EG_DB\990913a2\990913a2-026.scf’ (43>597)
CGAGAACCTGCAGGACTTAGTGGGGAAGCCGGTGTTCACAGTGGAGCGCATGTATGACGTGACGCCGCCTGGCGTGGTCATGGGGCTGGCCTGGACGGAGCAT
GGGAAGACTCCACGCTGTTTGTTGAGACGTCCCTGAGACGGCCGAGGGACAGAGACAGNGACAAGGGGGACAAGGATGGGAGCCTGGAGGGGACCGGCCAGCT
GGGGGAGGGATGAAAGAGAGCGCCGCATATCCTACACTTCCCCAGGCCCTCCTGATGCAGCACGACTCCGCCAACAGTTCCTGTTGAACTCCCCATCCACTGC
ACGTGCTGAAGGGCCACCCCAAGACGCCCGAGGGCCGCTGACATCTCTCNGNCCTCTCTCTGCTTTGACGGCAGGCNGAGAAACTGCTGACGCGAGGCTCTCC
AGCAAAGTGCTGTGGCGGTTAGAGAGACACCGCCAACGGCGGGACCGCTCCCTCACTAGACAGAGATCTTTACTGTTCTTTCCCAGCCCTGGGGCTGGGGCCT
CCCGATTTTCTTCGTTCGGGAGGGCGGGCCGGGGGGGGCG
>‘990913a2-028.scf” came from CONTIG 19 at offset 0;
“E:\SEQUENCE\export\EG_DB\990913a2\990913a2-028.scf’ (1>538)
ACGGGGGCGGCGTCTAACTAGNGGTCCCCCGGGCTGCAGGTATTCGGCACGAGGCTGTGCTGGACTGCATTTCCATGGAAGGGTGCATAGTCTGCTCTTTTTG
TCTGGA1TCCCTCATCAAGCTGGTCAGAGTCACGAACGTCGCCCTGTGTGGGTGTGGGCCGGACCCTGTCCTTCCCGTCACTGTGTGCCTGTCCCCGCCCACT
GCCCCAGGTCCTGGAGCTTCTTAGAAGTCTGAATGCCTGGNGGCAGAGCCTTCCGCTGTCTTCTTCAAGATCATCCGCGNGTCCTCACCTTTGTTTTTTTTTG
TCAATGTTCAAATCATACATCTATTTCTCCCGAGTGTGTGTGTGGTTTTATGCNTTGTTTATGTTAGATATTACATATGGGAAGAATGACTCTTGCATATGGA
AGTAAAGACATAATAAAAAATATACCTGGCAATTTGTGCTACTACAACAAAGGCTGTCGCTATTTTATGTTATTTAAAAAAAAACATATCAGCATTCACAAAA
TCTTATATTAAAATAAAAACT
>‘990913a2-029.scf” came from CONTIG 20 at offset 0;
“E:\SEQUENCE\export\EG_DB\990913a2\990913a2-029.scf’ (41>144)
TGGTTTTTATTGTAAACAATGCATTCATCGACCTTCCAGCCCCATCAAACATTTCATAATGATGAAATTTCGGTTCCCTCCTGGGAATCTGCCTAATCCTACA
A
>‘990913a2-031.scf” came from CONTIG 21 at offset 0;
“E:\SEQUENCE\export\EG_DB\990913a2\990913a2-031.scf’ (53>58)
CAAAGA
>‘990913a2-032.scf” came from CONTIG 22 at offset 0;
“E:\SEQUENCE\export\EG_DB\990913a2\990913a2-032.scf’ (52>59)
TTGGACGA
>‘990913a2-034.scf” came from CONTIG 23 at offset 0;
“E:\SEQUENCE\export\EG_DB\990913a2\990913a2-034.scf’ (46>624)
GTACGAGGCTAGGCCGTGCAGCTGCTGCCACCGCCGTCTGTCTGCCTGCCCTCCCGTCGGTCCACCCGCAGCATGAGCGGCCTGCGCGTCTACAGCACGTGGG
TCACCGGCTCCCGCGAAATCAAGTACCAGCAGAGTGAGGTGACCCGCATCCTGGATGGGAAGCGCATCCAGTACCAGCTAGTGGACATCTCCCAAGACAACGC
CCTGCGGGACGAGATGCGAGCCTTGGCCGGGCACCCCAAGGCCACCCCACCCCAGATTGTCAACGGAGAGCAGTATTGTGGGGACTATGAGCTCTTCTGTGAG
GCTGGGGACATACACACTGANGAGNTCCTGATACTGCCTGAGCAGCCAGACCCTGACTCGTCATACATTCCCTCCCACCATCACGCGGCTGAGGACCTGGACG
ACTCCTGTTTTCTACTGACGGGGCTTCCCTCACCAGACCCTCTCTCCTACTTAGCCCTCTTTCATCACACACATCTCACCACGCTAAATGATTAGACAGCAAG
GTGTGCTAGTGGCCTGGTGGCCTCTGCTGTGTGGCCTGTGTCATACAGTTTCAAGCGCATCGCG
>‘990913a2-035.scf” came from CONTIG 24 at offset 0;
“E:\SEQUENCE\export\EG_DB\990913a2\990913a2-035.scf’ (53>562)
GCACGAGGCCCCTTCAAGGCCAGAGTCACAGGTGACGGCCTCGTCAGCAACCACAGCCTCCATGAGACATCATCTGTGTTTGTGGACTCCCTGACCAAGACTG
CCACCATCCCCCAGCACAGTGCCCCAGGCCCGGGTCCCACTGATGCCAGCAAGGGGCTGGCCAAAGGCGTGGGGCTGAGCAAGGCCTACATGGACCAGAAGAG
CAGCTTCACGGTGGACTGCAGCAAAGCAGGCAACAACATGCTGCTGTGGGGCGTGCATGGGCCCCCGACGCCCTGTGAGAGATCACTGGGAACACGTGGCAGC
AGCTCTCAGAGTGCCTACTGCTCAGGACAGGGGGANACCGCTGTGGNCAGGGGGGGACAGCCATCCGGGCAGCCTCGAGCCTGGGGCTGGTGTGGCGCTCCAT
CACACTCCAGACAGAGGCCCTCGCTGCCCTGCACCACACACGCCCACCCGCAGCCTGCACCTCCTACCTGCCTCTGGTGGTACTGCTCANGGCAAGAG
>‘990913a2-037.scf” came from CONTIG 25 at offset 0;
“E:\SEQUENCE\export\EG_DB\990913a2\990913a2-037.scf’ (54>628)
GCACGAGGACCTCTTCTGCCCCCCGTGCCCTGTGCCGACCACCTGCTGTCCTGGTGCAGCATGTGTTACTTCTGGCTCCCATGCCCTAGATCTGCTGGTGGTA
ACCTTGGAAGAGCTGGCCAGGCCTGGAGGTTTCCTTCTCCATTTGTAGTGTICCAGAGTGCCATGATTGCCACGCCCCACCAGAGCTCCACTTGTCATGCTCG
CGGCCCATCCACACACCTTCTTGCCTCTTGCCTCCTGCAGGGGGGGGGGGGATGGGAGTGAGACAGCCCGGCCCCTCTTCCCCTCTCCTCCCACTGAGCACCA
GCTGCTGCTGCCAGNGAAGCTCATGACCGGGGCGGNCAGCTTCCCTCCACTGACGAATCKATTATCTTAACTAATAAATCACTATTAAAGACCAACTAACTAA
TGATGAAGATGGCGAACATTTAACTCGTTTTAGGTTTTGTTTCTAGTGCCGTGTTTTTTTACCTGGTTATATATTTTATAATTTACTGTGATGACAAGAATGC
TGTTCTTGAGACATTTTGATTTGTTATTNGTTTTCTTTCTTTCCCTACATCTACCATATC
>‘990913a2-038.scf” came from CONTIG 26 at offset 0;
“E:\SEQUENCE\export\EG_DB\990913a2\990913a2-038.scf’ (53>537)
GCACGAGGCAGCGGGCGAGCTGGGACCCGCGTGGCATCCTGCCTCCCTGCCCGCGAAGTGACAGTTTACAAAATTATTTTCTGCAAAAAAGAAAAAAAAGTTA
CGCTAAAAAAAAAAGCCAAAAATACCCACAAAACCACATATTCTATTATACAAAAAGTATTCTTTTCTCCACCCGCTTAAAAGGAAAAGAGGAAGAATTACCC
CTTTGCACCGCAATGTTTTGTTTTGCTGGGACATAAGCAAACACCCCAGCCAATGTTATATCCATCCTTTTTTTCGTTTTTTTTTTTCCTTTCTTTCTGCCCC
TCTGCTGNTCCATTCCCCATCTCCTGGCCCCTTGTGGGGAGTGGGAGGTGGGGCGGGGAAATCTGCCAAAGCCATGTGCTGTGTGTGCTGCCCCTGTCCTGAA
ATTTTTGTTTTAAAAATTTTGATTGTTGTTTTTAGAAAAAAAAAAAAAAACCCGATGAAGAAAGACCCTGACG
>‘990913a2-040.scf” came from CONTIG 27 at offset 0;
“E:\SEQUENCE\export\EG_DB\990913a2\990913a2-040.scf’ (54>148)
GCACGAGGGTTTGGGAGAGAGCCAGCTATTCTGGGGGGAAAATTTTGAGGGAAAACAACTATTAAATGGGGTGGATTTAGTTTTTTTTGTCCCTA
>‘990913a2-041.scf” came from CONTIG 28 at offset 0;
“E:\SEQUENCE\export\EG_DB\990913a2\990913a2-041.scf’ (1>634)
CGGGGGGGCCTTAAATATGGTCCCGGCTGCAGAAAATGAAGCTGAACAAAATCTTCATTGCGGAGGTACTTGCCTCATCCGAGCGAGCCCGCCGACATGCTGA
GCAAGAGAGAGACGAGCTGGCGGACGAGATTGCCAATAGCGCCCTCCGCAAGTCTGCACTTPCCGGATGAGAAGCGGCGGCCTGGAGGGCTCGATCGCACAGC
TAGAGGAGGAGGTGGGAGGGGAGCAGAGCAACATGGAGCTGCTCAATGACCGCTTCCGCAAGACCACACTGCAAGGGGACACGCTTGACACTGAGCTGCGGCC
GAGCGCAGCGCTGCCCCACAGAGCGTAATGCGCGCAGCAGCTGGAGGGGAGAACAAAAGTTGAAGCCAGCTGAGGAGCGGGAGGGCGGCAGTCCAGTTATGGC
ACCTCTTTGTTTGAAGCAGATGGGGTGGAGGGAGTGTGCAGGAGCAGGAAGACCGGCCATAATATCGGGTACGGAGAGTGTAGATTTTTGCGTGAGTGAAGCG
CTGCGTCATTTAGAAAGGGAGCATGCAGTGAAGTTAGGGGGTGGGAGGGGAGAGTCAGCCGCTTTGCCACCCGGGACGTGGCTGGCCCGGGCGCGGGCGCCGG
AAGGGGGGGGCGTGTCT
>‘990913a2-043.scf” came from CONTIG 29 at offset 0;
“E:\SEQUENCE\export\EG_DB\990913a2\990913a2-043.scf’ (1>368)
CGGGGGCTTTAATATGTCCCGCTAGAATGACAGTCCTGTGATGATAGGTGTTGTTTATGTCCGGACTTGTCTGTGCTGATGTTTTTTATTTGATCGGCAGGAC
AGAGGATTTTGGGGTAGTTCAAGAAAACAAAAAGAAAGGAGGAAGAAAAGTGAGCTCAACGAGCCCGCGTGCTTTTAAGAAACATGTTTGCCTGACAGCTGGC
CTGCCACGCAACAGATTCGCTTTCATAAGATTGCAACAACAAAGTTTATGTACATTAAATAAATAAAAAATGAAAAAACAACAAAATTTGAGTTGTGTATAAT
AAAAGGAGAGAAAAATAATGGTGTTTAGTAATTTTGTTGTTTTGTTTTTTTTATAATT
>‘990913a2-044.scf” came from CONTIG 30 at offset 0;
“E:\SEQUENCE\export\EG_DB\990913a2\990913a2-044.scf’ (46>224)
GCACGAGGCTCCCCCCGGCGCCGCTCTGGCCGCACTGCGCTCGCCCTGAGCTCCGGTCTCCTGCTAAGCCAGCGCCGCTGTCGCCTCCCTCCAGTCGCCATCA
TGATCATCTACCGGGACCCTCATTAGCCATGACGAGATGTTCTCCGACATCTACAAGATCCGGGAGGGGGCGGACG
>‘990913a2-065.scf” came from CONTIG 30 at offset 0;
“E:\SEQUENCE\export\EG_DB\990913a2\990913a2-065.scf’ (54>601)
GCACGAGGCCCCCCTCCCCCCGAGCGCCGCTCTGGCCGCACTGCGCTCGCCCTGAGCTCCGGGCTCCTGCTAAGCCAGCGCCGCTGTCGCCTCCCTCCAGTCG
CCATCATGATCATCTACCGGGACCTCATTAGCCATGACGAGATGTTCTCCGACATCTACAAGATCCGGGAGGTCGCGGACGGGCTGTGTCTGGGGGGGGAGGG
GAAGATGGNCAGTAGGACAGAGGGTAACATCGATGACTCGCTCATTGGTGGGAAATGCCTCGCGCTGAGGCCCCGAGGCGAAGGTACCGAAGCACAGTATCAC
TGGTGTCGATATGTCATGATCATCACTGCAGGTAACAGCTCACATAGAAGCTACAGAATACATCAAGATACATGAGTATCATGGAACTGAGAACAAACAAAGA
TAAATCTTTGAAGGCTGAGACAATAGCCATCTGTATTCAAACTTATTTTTGGAACATGATGAATGCTGGTGTTCGGCTCGGAGTGGACCTTTTATTTTTAGTG
TTAATGNAAGTTAAGATGGTTCTGTATACTGT
>‘990913a2-045.scf” came from CONTIG 31 at offset 0;
“E:\SEQUENCE\export\EG_DB\990913a2\990913a2-045.scf’ (1>208)
CGGGGGGGCGTTAGACTAGGGTCCCCGGCTGAGGAATTGGCACGAGCCCCCTCCCCCCTAGGCCCGACACCCCGTCTCTGGTACAAGCAGACCAAGAGGCTCA
AGCCATGGCAGACCAAAGCCGGGGGAAGAGATGCAAGCACAGAAGGCCTCCCGGGTAAAGATGTGACTGGTGGGCTCCTTTCCTGCTATACTGGGGAGGGAGC
AA
>‘990913a2-046.scf” came from CONTIG 32 at offset 0;
“E:\SEQUENCE\export\EG_DB\990913a2\990913a2-046.scf’ (1>454)
CGGGGGGGCGTTAGACTGTGGTCCCCGGCTGAGTATAACTTTTTTTTTCTTTCTTGTGAAGAGCTCATGTTAATAGAGAGATGGAGCCCAAGAACAGACAGCT
CGGTTTCGAAATACAAGTTTAGGAAAATCTTATCTCAGTCATGCATAAATATGCAGGGATATGGCAGAAGACACCAGAGCAGATGCAGAGAGCCATTTTGTGG
ATGGATTGGATTATTTAATAACATTACCTTACTGGGGAGGGAGGATTGGAAAAAAAAATGCCTTTGTGACAGCTTCTTATCTTTTTATTGTTGTTTCTTCTTG
TGGTCTTGTATGAGTGTGAATCATTCCTTCTTGTTATGNTNATTNGTAGTTNCAGTGAATGTGATACTTTTTATGATTTTTTCAAGTTTGAAACTTTATCAGT
CTGTGTATAGACATACGTTTNCTTTAAAATTATGCGTGTAA
>‘990913a2-047.scf” came from CONTIG 33 at offset 0;
“E:\SEQUENCE\export\EG_DB\990913a2\990913a2-047.scf’ (50>534)
GCACGAGGCCGATCTCTGCGAAGAGCTGGAACCCCGGGACCTTGGCGAAGTCCAAAATAAAGAGTGTCGAGACGCCTGGTGTGTGTTGGTGTTGTTGTGNGCG
TGCACGCCTGTGTGGGTGTGGGGTGTGTGTATGTGTGGTGTGTGTGTGTGTCTCTTTGTGTGGTGTATGTCTGTGTGTATGTGGCTGGGTGTGTCTGTGAGAC
TTGCCCGCGCGGTCTTGNGCCCCGCGCTTCCCGGGGCGCAGGTGCATTGCAGACGGCACTGGGGTCCGCCGAGGGGAGCCTGGTGGTGATTTTGGTCCTCCTT
CACCGCCCGCCAGCCCTGGGAGAGCCTTTGTTCAGCTGATGTGAGGGCTCCTGTTCTAGTCATGCCTGTCTGGACAACGATCAAGTCCGAGCACGCGGGGAGG
CCCTAAACCCACTGACCTCAACCAAAAAGGAGGCGGACAACAAATCTCCCACCTGGGTGTGGGGTTGGGAAGG
>‘990913a2-049.scf” came from CONTIG 34 at offset 0;
“E:\SEQUENCE\export\EG_DB\990913a2\990913a2-049.scf’ (8>579)
CGGCGTCTATAATAGNGGTCCCCCGGGCTGCAGGCGGGTGCACGGGTGGCGAGCGCAGACGGGAGAGGCTGCCAGACGAGCACCATGGCTCCCTGCCCGCTTC
GCCCGCTGCTGTGGGTCCTCGTGCTGGGGCTTGGGCTGGCGCTGCTGCGCGCCGCGGGCGGGGAGGGAGTGCCAGGCACCACCCCCTGCTCTCGCGGGACCTC
CTGGAGCGCGGACCTAAACAAGTGCATGGACTGTGCCTCGGGCCGGGCGAGAGCGCACAAGGACTTTTGCCTGGGCTGCACTGCGCGCCCTCCAGCCCCCCTC
TTGTTGGGTGGGGCATTCTGGGGGGGCCCTGGGCTGGCCCTGGGCTGGGGTGGTTTTTGGTGCTGGGCTGGGAGGGGGCGCAGAAAAGAATTTTACACCCCAT
CGGGGACGGGGGGGAGTGGCTGGGGGGCCTGTCAGGCGGGGCGCCCCGCCGGAGAGGTGGGCCGTCTTTTTTTTTTTTTTTGTGCTCCAACTCCAACCAACAA
GCAGCTGTCTACACAGGGGGGAGATGGGAAGGATATTTTTTAGTTGTCCATTTCAAG
>‘990913a2-052.scf” came from CONTIG 35 at offset 0;
“E:\SEQUENCE\export\EG_DB\990913a2\990913a2-052.scf’ (8>705)
CGGCGTCTATATTATCTGTGGCTCGGCCTGTTTGCTGCTCTCTAACATCCATACTTGTACTGTACTTTCACCTGTGGACCCTCTCCACATCAGACTTTTACTG
TCCCTTATGGTTGTATACATTCCAAACCTATTCCAGTTGGTCCCAAGTGTCCATCCCTTATAACTCTGCCCTTGCTATTTAACCTCCAACTAAGGTGGTCTAG
ATTTCCCTGATGGCTCAGCTGTGAAAGAATCCATTTTCAGCGCACAGGAAACCAGAGACGCAGGTTCAATCCGTGGGTCGGGATGATCCCCTAGAGAAGGAAA
TTGTCTCCCACTCCAGTITTCTTGCCTGAAAAATCCCTGGACAGAGGAACCTGGCTGCTTCAGCGCAGAGGGTTGCACAGACTCACACCGACTGACCACCTAC
CACATTGCTATGTGCTTTCTTTTTTATCTTCTCATGCTATTGCCAACCTGAATGCGACCTAACAGACCTGAAGAGGAATAAGAGAAATCTTCTGACGAAAGCA
TTAAAAAGCAGAAAGCCUAAATCGAATATCTTGAAAGACTCATACATTAGAAATTGCTATGTTCTTAAACTCAACATTTCTCTTTTACCTCAACCTAAAGAAC
CGTCCGTTCCTGTTTAGGGGAAATAAAAATCACACTGCCACCTAACCAAGGAACATAAACATTTACTTCCTCCTCCTCT
>‘990913a2-051.scf” came from CONTIG 35 at offset 34;
“E:\SEQUENCE\export\EG_DB\990913a2\990913a2-051.scf’ (47>556)
CTGCAGTCTAACATCCAGCCTTGTCCTGTACTTTCACCTCTCACCCTCCCACAGCAGCTTCACTGCCCTTATGGTGTAACATCCAACCATTCCAGNGGTCCAA
GAGCCACCCTACAACTCTCCCTTGCTATTTAACCTCCAACTAAGGAGGTCTAGATTTFCCCTGATGGCTCAACTGTGAAAGAATCCATATACAGNGCAGAGGA
GACAAGAGACGCAGGGTCAATCCGCGGGTCGGGAAGATCCCCTAGAGAGGAAATGGTAACCCACTCCAGTATTCTTGCCTGTAAAATACATGGACAGAGGAGC
CTGCGAGCTCAGTGCAGAGGGTTGCAAAGACTCTACACGACTGAGCACTANCACATGCTTTGTTGCTTTTTTTTTTTTATCTTAGTTATTGACAACATGATGG
ACCTACAGTACATGAGATGGATACAAGGAATATTTGTCGGAGCAATAAGAAGTAGAAGGATTGATTGAATATTTTTAATGACTATACATTATAAATTTC
>‘990913a2-053.scf” came from CONTIG 36 at offset 0;
“E:\SEQUENCE\export\EG_DB\990913a2\990913a2-053.scf’ (48>595)
TCTTGTTGAAATGAAATACCACAACAAAAAGCAAGGAACAATGAGCAAGAGAAAGGAGAGCTTTTAAAAAATTAACTGGACCACTTTTGGGGGTTGGGGAGCA
GAACTGCTTTTGGTGATCTCACGTGACGTGTGAAGGTACACTGTGCTTCTTTGCTTCAGGAAATATAGGGTTGTCTGGAAGGAACCCAAGGTAGAGCCAACGG
GGGGGGGGTTTTTTCAAATCCCAAACTCACCGACAGGCGTGAATGTTCCTTGTAGTCCTCTGTAGACTGTGTCCTGGCCCTGTCAGCCCTTCTTCAGCCTGCA
CCCTGTTCCAGAGCCAGTGAGACGGAGACACTGTGATGGAGCTCTCACTTCCAAGTGATAGTATTATTAATTTGTGTGTGTCAGACTATGACTCAGATGTGGG
TGGATTTTTATTTAACGATAATGACATTATAAAACTTTATTTGTATTTGTATTTAATATCATTTCATTTTTAATCTTTATTTGAGCACGTGTTAAAATTAATA
CTTTGGTTTATGTTCTGATTTTNTTTTTACTCTA
>‘990913a2-054.scf” came from CONTIG 37 at offset 0;
“E:\SEQUENCE\export\EG_DB\990913a2\990913a2-054.scf’ (52>252)
GGGCACGAGGTAAACCCAAGCCCTTGACCTCTTACAGGAGCTTTGTCTGCCCTCTTAATAACATCGGGCCTAACCATGTGATTTCACTTTAACTCAATGACCC
TGCTAATAATTGGCCTAACAACAAATATAGTAACAATATACCAATGATGACGAGATGTTATCCGAGAAAGCACCTTCCAAGGGCACCATACCCAGCTG
>‘990913a2-055.scf” came from CONTIG 38 at offset 0;
“E:\SEQUENCE\export\EG_DB\990913a2\990913a2-055.scf’ (277>558)
GAGGAGAAACTTCACGGNGGTCCCCAGNTGGATTTATTGTTGAAGGAGAACCACCAGGAAAGGGTTCCCACTTGCTCTTGGTCTGCTGGCCACCTGCTCTCAA
AAAATACTACCTTGCAGACAAGATGCTACGGATGAGGAACTGCAACGAAAGATTTTTTGAGGTCCAGATGGGATGTGCGAGGCACACGATCACTCCTGTTGTT
GTGGTGTGTAAAAAAGATGGAAAGAAATATAATTAAAAAAAAACACTGCTGCTTTTTGTTTGTCTGGGTTGGGG
>‘990913a2-056.scf” came from CONTIG 39 at offset 0;
“E:\SEQUENCE\export\EG_DB\990913a2\990913a2-056.scf’ (46>553)
TGGGACGAGGGTCAATCCTAGACATTATAATGCATGGTATGGTTTAGGAATGATTTATTACAAGCAAGAAAAATTCAGCCTTGCAGAAATGCATTTCCAGAAA
GCACTTGACATCAACCGTCAAAGTTCAGTTTTGCTTTGCCACATTGGGGTAGTTCAGCATGCACTGAAAAAATCTGAGAAGGCTTTGGATACCCTAAAGAAAG
CCATTGTTATTGATCCTAAGAACCTTCTATGCAAATTTCAGAGAGCCTCAGTTTTATTTGGAAATGAAAAATATAGTCTGCTTTACAGAACTTNGAGAATGAT
CACATNGTTCCCAANGATCCCTCGTTACTCTTAATAGAAGGTTAAAGAGTTAGTCAACGCACTCGCCTGTGATNTCTTTGGGTTGGGTAAATCCTAAGACCAA
TACCAATAAAAGCAATGTAACCTACTTCAAGAGAGAGCATACCCAGAGAAAATATGGACAAGATCCAGAGAGAGAG
>‘990913a2-057.scf” came from CONTIG 40 at offset 0;
“E:\SEQUENCE\export\EG_DB\990913a2\990913a2-057.scf’ (48>291)
TCCATACGGCGTLTGTCTTGAATTCCCGTCGTAACTTAAAGGGAAGCTTTCACAATGTCCGGGAGCCCTTGATGTCCTGCAAATGAAGGAGGAGGATGTCCTC
AAATTCCTTGCAGCAGGAACCCACTTAGGGGGCACCAACCTTGACTTCCAAATGGAACAATACATCTACAAAAGGAAAAGTGATGGCATCTACATCATAAATC
TGAAGAGGACGGGGGAGAAGCTTCTGTTGGGCGCTCGGC
>‘990913a2-059.scf” came from CONTIG 41 at offset 0;
“E:\SEQUENCE\export\EG_DB\990913a2\990913a2-059.scf’ (46>612)
TGCTGGTGCTGTTGGCCCAAGAGGTCCCAGTGGCCCACAAGGTATTCGAGGTGACAAGGGAGAGCCTGGTGATAAGGGTCCCAGAGGTCTTCCTGGCTTAAAG
GGACACAATGGGTTGCAAGGGCTCCCGGGTCTTGCTGGGCATCATGGGGATCAAGGGGCTCCCGGGGCTGGGGGTCCCGCTGGGCCCAGGGGCCCTGCTGGTC
CTTCTGGCCCCAGCTGCAAAGACGGGCGCATTTGACAGACTGNNGCACGTGGACCTGCTGGCATTTCGTGGCTTCAAGGTAGCCAAGGGTCTGCTGGCCCTCC
TGGCCCCCTGGCCCTTCTGACCTCCTGGCCAAGGGGGGGGGTTCGATTTGGGTTTGTGTGACTTTTCAGGCTGCCCACCCTCCTACTACTCTCTCNACCCAGA
TTTGAANTGTGCACTTGTATCTTCACAACAATTGAAACTTTTTTTCAAAGCTTTAGAAAACCCCTCGCATCCCGACTTGACCACCCCCCAAGGGACTGTTTTA
TTGTTGACTATCCAAGTGTCTTGTTGTTTAAGTTTTTTTTTTTTTGCGAACT
>‘990913a2-075.scf” came from CONTIG 41 at offset 534;
“E:\SEQUENCE\export\EG_DB\990913a2\990913a2-075.scf’ (48>547)
CGGACATTCAGACAGAACGTGCGTACCAAAAGCAACCGACCATCTTTCAAAATAAAAAGAGGGTCCTGCTTGGAGAAACTGGCAAAGAAAGCTCCCTGATACT
ACAGAACTTGGTCTGGGCTTCAAGACTCCAAAGGAGGCCATCGAGGGCACCTACATTGACAAGAAATGCCCTTTTACGGGTAATGTCTCCATTCGGGGCGGAT
CCTGTCTGGCGTGGTGACCAAAATGAAGATGGAGAGGACCATCGTCATCCGCCGAGACTACCTTCACTACATCGAAAGTACAACCGCTTTGAGAAGCGCANAG
AACTNCCGCGTGCACCTTTCTCCTGCTTAGGGACGCCAGATCGCGACATGTCACGTGGGCGAGTGCGGCCCTGGCAGACTGGCGCTCATGTCTCAGGTCACCA
GCTCCGCACAAGAGCATTCGAAGTTGAATGGCCTTGCTGTCCCAACACATAGTATTNCATTCAAAAAAAAAAAAAAAAAAATAAAAAT
>‘990913a2-060.scf” came from CONTIG 42 at offset 0;
“E:\SEQUENCE\export\EG_DB\990913a2\990913a2-060.scf’ (52>263)
GCACGAGGCCCTTCTTCACCCCTTGCCCCTCGCCATCACCCCGACTCTCAACACAGCACAAACCCTGCAAACCCAAAGAGAATATTAATACTTGAAGCAAGAA
GGGTGCATGCCAGTCCCTCTCAATCATGGCCAGGGGATGCCAGGGCTTGCGCCCCTGGACTCGTAGCCCTGCCCAGGGCAAGGAGGGCTTTTCCCTAGAGCTA
CTGGGC
>‘990913a2-061.scf” came from CONTIG 43 at offset 0;
“E:\SEQUENCE\export\EG_DB\990913a2\990913a2-061.scf’ (54>571)
GCACGAGGGACTGTTTTCATCAAGAATAGTCTCTTGAGAAAACTTCAAATTTACGCTAACAAAAGGTACATTTGCCTCAGGGATGTCAGTATTTTTTAGAAGC
TCCACACATGGCTGTATCATAAAGAAAACTTAGTTGAATTATTTTGACTGCTGTTTTCTGCCCTGTACTGGAGCTTTAGGTTGCTTGTTGACTTTGGGACAAC
TTTGGGTGATGAGGCTGCAGCTGATTTCTATCTTGATTCATCAGGTACGGGAGTTTGAGGGCGCAGTCATAGCTCTCTTTATTTACTGGCCACCNAGACAGAC
NAAAGTCGAGCCCTGAGGGAGGCTTTTATCGTCAGAATTTGCCCATCTCATGACCTCATGCCACAGTTTTCGTGTTCGTGTTGTATATTTTTAGGTTCCGTGG
GACTGCCATCAGTCAGCCGACCGCGGCAGACAGCACTACCCCTCAGCTCTCTAACTCCACATCCTATATTCTCTGCGCCTGTGCCACCTGCTGATTTCAATGC
TT
>‘990913a2-062.scf” came from CONTIG 44 at offset 0;
“E:\SEQUENCE\export\EG_DB\990913a2\990913a2-062.scf’ (49>602)
GTTTCTGCGACCTGTTCTCGAACGCTCAGCGTGGTCCTCCTTCCGATTTGGAGATGCCTGTCTGGGGGAGGTGGCAACAAGTGCGGGGCGCTGTGGGGAGGAC
ACGGTACCACGGCCGAGGAGGTGCAGNGTGACGGCCGGAGCTTCCACCGCTGCTGCTTCCTGTGCATGGTTTGCAGGAAGAATTTAGATAGTACAACAGTGGC
AATTCACGATGAAGAAATCTACTGCAAATCCTGCTACGGAAAGAAGTACGGGCCAAAAGGGCTACGGGTACGGCCAGGGCGCAGGCACCCTCAACATGGACCG
GGCGAGAGGTTGGGCATCAAGCCGAAAGTGTTTCAACCTCACAGACCGACACAAATCAAACACTTTAATTTGTCAGAATTGGAGGTGCTGGAGTGTCAGATGT
GGGTTCCGTTCGCTNCGATAGACTTGTAGCTGAAGCCTGCACAAACTGTTCGTGTGCTAGGCGGGAGGTCTAGTCTACACTTGATGAGAAAAGTGAATTTTGT
AGATGTCCGAGACTCGCCCAGTTTGGTTCGCAGGCTGGCC
>‘990913a2-063.scf” came from CONTIG 45 at offset 0;
“E:\SEQUENCE\export\EG_DB\990913a2\990913a2-063.scf’ (47>258)
GCACGAGGGAAAGTTCAGACATCAGGTAAGTGTGTTCTAATTAAAAGATCATGAGTTCAAATCCCAAGCAGGGTTTTAGCTGGATTTGAGTCCTGGCCATGTG
GGTTTGAGTCCCAAACTAGGTTTTTGGCTAGATTCAAATACTGGCACATGGGTTCAAGTGCCAATATGAGGTAAATGGNTTTAAGACCATTTAGGTTTTTGTA
TTCTTT
>‘990913a2-066.scf” came from CONTIG 46 at offset 0;
“E:\SEQUENCE\export\EG_DB\990913a2\990913a2-066.scf’ (47>584)
CGCCGGCCCCAGCGCGCTGCCACCGCTGCCCACCCACCATCAACCAGCATGTCCTCCGCTCACTTCAACCGAGGCCCCGCCTACGGGCTGTGGGCTGAGGACA
AGAACAAGCTGGCCCAGAAGTATGACCACCAGGGGGAGCAGGAGCTCCGAGAGTGGGTCGAGGGGGTGACGGGGCGCCGCATCGGCAACAACTTCATGGACGG
ACUAAAGACGGCATGATTCTTTGTGAGTTCATCAATAAGCTCCAGCCAGGCTCCGTGAAGAAAGTAAATGAGTCCACTCATAACTGGCATCAGCTGAAGAACA
TCGTCAACTCATCATGCCATCACCAGTCGGGGTGAAGCCCTACGATATCTTGAGGCCACGACCTGTTCGACACACCACCAACGCAGTGCATCACCTTTGCGCT
GCCAGATGACAGAGAAAGGACAGGNGATGGGGAGGAATAGCGGAAGCGTACGTGATTGAGCAAGAGCTAAAGAAGCGACATTNGTGTCAGTGGACCCAGTGCA
GCAGAGCTGAGGTAGGGCCGCG
>‘990913a2-067.scf” came from CONTIG 47 at offset 0;
“E:\SEQUENCE\export\EG_DB\990913a2\990913a2-067.scf’ (290>553)
AACTTGAGTACTACACAAGCTGGCTGGATACTGGGCCAGCATCACTCCCACGCTCCTTTCACTGTACCAAAGACAGAGAAATGTCAGTTGGGAGCCAGGGACA
TGTCATGTCCGCAGTACAACTTGATCTGATGTGTGTGAGAGGACGTTCTTCATTCTCCACTTGATACAGCACCTATCGACAGGACCCCACCCACTTTGTTTTA
TGATTACAGTTTCGAGTCCTTGCCTTCCTCCCACCCAGATACCTGACTTACCTCCCT
>‘990913a2-069.scf” came from CONTIG 48 at offset 0;
“E:\SEQUENCE\export\EG_DB\990913a2\990913a2-069.scf’ (48>147)
GCACGAGGCCCCGCTGAGAGGAGGCAGCCAGTGCGACTCCCACCGCCCGGCGACCCACCTTCCTCCAAGTCCGACGGGCAGCCGCCCCCGGGGCGACAAG
>‘990913a2-071.scf” came from CONTIG 49 at offset 0;
“E:\SEQUENCE\export\EG_DB\990913a2\990913a2-071.scf’ (55>586)
GCACGAGGCGAGTTCGACGGGAGAGGTTCCAGAATGTTGCCAAAGAAGGAGTGAAGTTTGATGAAAGTGAGAAAAGTAAGGAGAGTCGTGAAGCAGTTGAGAA
AGAATTTGAGCCTCTGCTAAACTGGATGAAAGATAAAGCACTCAAGGACAAGATCGAGAAGGCTGNGGNGTCTCAGCGCCTGACAGAGTCTCCGGGTGCTCTG
CCAGCCAGCCAGACGGGCGGGNCTGGCACATGGAGAGGATCATGAAAGCCCCAGCATACCANACAGGCATGGACATCTCATCCATTATTATGCCACCAGATAA
AACATTTGTTTTAACCCACCACCCACTATCATAACTGCTTGAAGAGTTAGAGAGAGATGAATACTGTCAGATCTGTGTGTTTGTTGAACACACCTCGTCTGAT
TCTTTGCAACCTAAGATTGAGAAGATANATGATCTCGCTCACTAACATCACTGTGCAAGTGAGAGACCAGAGACTGAGACACAAGCACGAAGAACAAAGATAA
AGAGGAGACAGACGAC
>‘990913a2-072.scf” came from CONTIG 50 at offset 0;
“E:\SEQUENCE\export\EG_DB\990913a2\990913a2-072.scf’ (55>600)
GCACGAGGGCCTGCCTGCCCAACCTGGACCGCGTGGGCCACCCCAAGGAGCGCTGCTICGCTTCTGATGGACGGGCCCTGCTGCCCGCTTTCACTGTACCCTG
GCCACACCCACCCCTCCTGCCGCTGTTCATTAACACCACCCGCCCCACGTCTCCAGCTGCGGACGGGTTCAGCCTGGCCTGCCCGTGCCTGGGAGGTCACCCC
GCTGGGCTGGCCCCTGAGGGGCCCCTTTGGGAGCAGGTGTGTGGTACAGGTGGGCTGCTGTTGGCCACCTTTAAGACAGATTCCTGTCTAGGCCTCGCCGGAG
ATGTTCCTTCCCTCTCGNCCCTCCACATACACACTACTGGTGGAGGCTGCCAGCACCATGCTCCCAACTCCCAGACATCCACACCTGGAAATTAGAGAAAGTA
CAGCAACCTTACTACAGCGCAGCCTCACCACTGAGACATGNCTNCNCCCTGCTGTTAGACCCGCCAGCTTCAAGTGAACTATGTACTGAACCATAGTCCATGG
ATGGCTGTGGTTGAGTGNCNACACCAAGCC
>‘990913a2-074.scf” came from CONTIG 51 at offset 0;
“E:\SEQUENCE\export\EG_DB\990913a2\990913a2-074.scf’ (55>269)
GCACGAGGATAAGACCAATTGGTATAAAACAAGATTACTAGGAGGAAAAAGAAAATATTGTGGTTTAGAACTGGTACTGGAGAAGGCAATGGCACCCCACTCC
AGTACTTGCCTGGAAACTCCCATGGATGGAGGAGCCTAGGGGGCTGCAGNCCATGGGGGGTGCTAAGAGTCAGACATGACTGAGCGACTTCGCTTTCACTTTT
TACTTTC
>‘990913a2-076.scf” came from CONTIG 52 at offset 0;
“E:\SEQUENCE\export\EG_DB\990913a2\990913a2-076.scf’ (49>616)
GAGCTCCAGGACCGACAGGATGGGGTCAGCCTGCATCAAAGTCACCAAGTACTTCCTCTTCCTCTTTCAACTTGCTCTTCTTTGTCCTGGGCGCGGNGATCCT
GGGCTTGGGATGTGGATCCTGGTCGACAAGAACAGTTTCATCTCCATCTTACAGACCTCCTCCACCTCGTTCAAGGTGGCGGCCTACGTCTTCATCAGACGCG
GGGGCGCTCACCATGCTCATGGGCTFCCTGGGCTGCCTGGGNGCCGNCAAAGAGGTCCGCTGCCTGCTGGGATGTACTTTGCCTTCCTCCTGCTGATCCTCAT
CGCCAGNTGACTGCCCGGGTCTTTTTCTATTCAACATGGGCCAGCTGAAGCAGGAGAGGGCAGCATCGTGACAAACTATTCACACTACACGACGGCTCGNGAC
AATTGCAGAGCCTGGATACTGCAGCTTAGGAAGTGTGGGCTGGTCAATACTCATGNAAGAAAACGACTATGATGACACACACTTCCTGTCTGGAAAATGAGAG
AGAGCTCTGAGCCAGAGGTNNTTGAGTCTTGCACAGACAACGCAAACCCAAAAG
>‘990913a2-077.scf” came from CONTIG 53 at offset 0;
“E:\SEQUENCE\export\EG_DB\990913a2\990913a2-077.scf’ (53>548)
GCACGAGGCTGAGGCTCGCTCTTTGCTCTGGGGATTCTGTGGCTTTTCCCAGTGCTAACCCACTCAACAAGAAACGGTTTACCAGACCATACAGACCCAGAAG
ACAATGAAATTGTCTGCTTCTTAAAAGTGCAAATAGCTGAAGCAATTAACTTACAAGATAAGAACTTAATGGCTCAGCTTCAGGAAACAATGCGTTGNGTGTG
CGGCTTTGATAACCGGACCTGTAGGAAGCTGTTTGGCGTCCATCGCCGAGGACTACAGGAAGCGGGCCGCCTACATTGCTTACCTCACTCGCTGTCGACAGGA
CTGCAGACACGCAGGCTACCTGGAAGGCTCTGCAGAGGTTCTGCGGACAAGAGTGGCCATCCTACTTACCACGCTGTGTGAGTGTGCTTGGAGAAGAGAGAGA
TCAGGAATATTCAGATTTATAATACAGCGTGTGTAAACCCCAAGTGAGATTTTGCAGTCTGTCGGCGATGCCAGAGCATTGGANA
>‘990913a2-078.scf” came from CONTIG 53 at offset 0;
“E:\SEQUENCE\export\EG_DB\990913a2\990913a2-078.scf’ (49>453)
GCACGAGGCTGTGGCTCGCTCTTTGTTCTGGGGGTTCTGTGGCTTTCCCAGCGCTAACCCACTCAACAAGAAACGGTTTACCAGACCATACAGACCCAGAAGA
CAATGAAATTGTGTGCTTCTTAAAAGTGCAAATAGCTGAAGCAATTAACTTACAAGATAAGAACTTAATGGGTCAACTTCAGGAAACAATGCGTTTGTGTGTG
CCGCTTTGATAACCGGACCTGTGGGAAGCTGCTGGCGTCCATCGCCGAGGACTAGAGGAAGCGGGCCGCCTACTTGCTTTCCNACTCGCTGTCGACAAGACTG
CAGACACGCAGCTCACTGGTAGACTCTGCAGTGGTTGTGNGGGACAAGAGTGGCCATCCTACTTACCACGTTGTGTAGTTGTGCTGGAGAAGAGAGA
>‘990913a2-079.scf” came from CONTIG 54 at offset 0;
“E:\SEQUENCE\export\EG_DB\990913a2\990913a2-079.scf’ (49>483)
CCCAGGCCCAGGCGGCCTGCCTATCCCCTCCTACTCCCGTCGGTGCCTCTCCCACCCCTGTATCTGCGCCCGGAGCCAGTTTGTTTGTCCAGCGGCGGCCTCC
GCCGCTCGAGCTGTTTGCGGAGCTTAGCGCGCCGCCGCGCAGCGCCGGCCCAATTAAGTCTAATTCATTATTCAGCGCCCCAGTGCTCCCGCGCCCAGCAGCT
GGCGGCTCCAGCGTCACTTTGATGCCCCTCCCAGCCCTGGCCCCGCCGTGAGTGCTAGGGAGCAGACAGAGAGAGCTGCTTTCCCTTCTCGGACCGAGAAGAG
ACTGAGGGGGTGGGATGTANNGAGGACAAATCTGTGACTGTGGGAGCGACGCGCAGCGNTCGACCACCCCACCACATAAAATGGAACTGANATCACCTTTTTT
TAAAGAGGAACGGCCCCCCTACT
>‘990913a2-080.scf” came from CONTIG 55 at offset 0;
“E:\SEQUENCE\export\EG_DB\990913a2\990913a2-080.scf’ (54>301)
GAACGAGGGCGGGAGCTGGGGTCTCTGGAGCGGGATGGCAGCGGGAGCCGGCCGGAGTTGGTCCCTAGGCTCACAGATCCCGCTCTCTGGCCTGAAACATGGC
CCGGGGGACCTGGCCCTCTAACCCGGCCTCGCCCCGACACGGCCGCCATGCCCAAGAGAGGGAAGCGACTCAAGTTCCGGGCCCAAGACGCCTGCTCAGGAAG
AGTGACCGTGGGGGATTATGCCAACTCGGATCCGGCAGGCGG
>‘990913a2-081.scf” came from CONTIG 55 at offset 0;
“E:\SEQUENCE\export\EG_DB\990913a2\990913a2-081.scf’ (54>687)
GAACGAGGGCGGGAGCTGCGGTCTCTGGGAGCGGGGTGGCAGCGGAGCCGGCCGGAGTTGGCCCTAGCTCACAGATCCGCTTTGGCCTGAAACATGGCCCGGG
ACCTGGCCCTCTAACCCGGCCTCGCCCCGACACGGCCGCCATGCCCAAGAGAGGGAAGCGACTCAAGTTCCGGGCCCAAGACGCCTGCTCAGGAAGAGTGACC
GTGGGGGATTATGCCAACTCGGATCCGGCAGTCGGGAGGGCTGGAAGGGGCAAGAAAGCTGGTCGCCAATGCTGTTCATAAGAAGTAAAATCTCTCTGTGGCT
TGGAAGCCTCCCAGGTTCCGGCAGTGGTAGCTCTTTCTGGGGCTGTGAGCCCTGGACACATCGAAGAAGAGGGAACTGATGCCCAGAGAAAGATACATGAGAG
ACTACTACAAAAAAGAAAGCAGAGAACAAGAGACCGAGGNACGGAGAGAGAGATCCAGGATTTTGGTTTGTGCCTCTTATCGACTGAGAATTGGATTTTCCTG
ATTGAGACGCCGACTGCCTGCCCTGTGCTTTGACAGGGACGAACATAACACGATCCTFGCCTTGCCTTCACCGACAAGAAAAGACGCTCGCTGGGTCGCCTGC
CATTTGGCTTCGTCGTT
>‘990913a2-082.scf” came from CONTIG 56 at offset 0;
“E:\SEQUENCE\export\EG_DB\990913a2\990913a2-082.scf’ (55>136)
GCACGAGGCTTCTCCAAGCATCACCCTGGGGAGTGTTTTCTAGACTTTTTCTCATACATGGGGAGCAGTAAGGTTTATCTAT
>‘990913a2-084.scf” came from CONTIG 57 at offset 0;
“E:\SEQUENCE\export\EG_DB\990913a2\990913a2-084.scf’ (42>252)
ATAGACCTGTGGTCTPACATACACGCACCCACACACACACGCATTTATGTTTTTTTTTTTTATTTAGTGGCTTGAAAAATGAAGAAAATAATGTTTTTTGCTT
TTATGTGGGAAATCATGGAATACCATACGAGTTAAGGCGTTCCTCTTTTCCTCTTCGCTCACCCACGGGGTGACACCCACGCTCCCCCACACCACTCATTGTT
GGGAT
>‘990913a2-085.scf” came from CONTIG 58 at offset 0;
“E:\SEQUENCE\export\EG_DB\990913a2\990913a2-085.scf’ (55>578)
GCACGAGGGCCCGGCGGAGTGGGGGCTGGGGGTGGCCATGGATGATGATATFTGCTGCGCTCGTGGTCGACAACGGCTCCGGCATGTGCAAGGCCGGCTTCGC
GGGCGACGATGCTCCCCGGGCCGTCTTCCCGTCCATCGNGGGGCGCCCCCGGCACCAGGGCGTAATGGTGGGCATGGGCCAGAAGGACTCGGACGGGGGGATG
AGGCTCAGAGCAAGAGAGCATCCTGACCCTCAGTACCCGATTGAGCACGGCATCGTCACCAACTGGGACGACATGAGAAAGATCTGGCACCACACCTCTACAA
CGGGTCCGTGTGGGCCCTGAGAGCCCCCCGGCTGTGACCGAGCCCCCTGACCCCCAGCAACCGGAGAGATACCAATATGTTTAGACCTCACACCCTCCATTAC
CGGCCTTCAGTGGCTGCCTGTTGCCTTGCCCACCACGATCGGAGGATCGTGACGGTACCCCACGGCCATTAGAGTTACCCTTCCTGCATCTCTTGACCGGTGC
GGACTGCGACA
>‘990913a2-086.scf” came from CONTIG 59 at offset 0;
“E:\SEQUENCE\export\EG_DB\990913a2\990913a2-086.scf’ (49>646)
GGAAAAGGACCGTGGCCCTGAGGGCCAGTTTGTGATGGGTTATGTGGTACGACTGCACAATTTTCACCAGCTGTCTGCACCCCAGCCCTGTTTTACCTTCTGC
CATCCTAACAAAGATGCTATGATTGACAACAATCGCTACTGTACCTTGGGGGTTGCTGTGGAGGGGGACACAGGGCTGGGTGGGTTTGCAGGCTACTTGAGAC
TGTGCTTTATCAGGACATCACTCTGAGTATCCGGCCAGAGACTCACTCTTCTGGGGTGGTTTCATGGTTTCCCATCCTCTTCCCCATTAAGCAGCGCATTACG
GGGGCGGAGGCCAGACCTCTGGTGCGGTGTGGCGTGCAGCACTCTAGAAGGGGGGATGAGGGGCTGGACGGACGGGCTGCTTGCTTCACACCCCACAGCGTCT
ACACATNGCTCTACCCGGCGAGGGCGAAGCGGGAGAGCGAGATTGTTCGAGCGAGAGGGAGACACTGGCTTGTCGCGGCCATCAAAGACTTAGCTGCTTCTCC
TCTCAGAGCAAGGGATGGGCCAGCACCACGGAGATAGCCGGAGGGATAGTGTTGGTTCACGCTCGCCAGCCCGGAGCGGATG
>‘990913a2-088.scf” came from CONTIG 60 at offset 0;
“E:\SEQUENCE\export\EG_DB\990913a2\990913a2-088.scf’ (217>525)
AGCATGATAGACAACTTCACTCCTTTGCTCCCCCAGGGCCACCCACCCCACTCACACCCAGCGAGGGACTTCCCCACCCCCACGCAGGGGGTAACCCTGACAG
CCCAGGAGGACTCACACCTCCTCCGTCCGGCTGAGCCCCTTGGGTCCTGGGAGAGACTCTTCAGCTGGTTCCTCCTCGATCCTCGCCCCCCGCCCGGNGGGAC
GGGGTNGCAGNGAGGCGGGTGTGGCTGAGGGGGGCTCGGGCTGCCCGGCTGAGGGTGGGCGGGGGGGGGTTCTTCTGGCTGGAGATGGGGGGTGGTCTGTTAA
TC
>‘990913a2-090.scf” came from CONTIG 61 at offset 0;
“E:\SEQUENCE\export\EG_DB\990913a2\990913a2-090.scf’ (56>651)
GCACGAGGGTTTAAAGTAAGCTCTGTTCCTTTCAATTCAGTCGTTCAGTTGTGTCCGACTCTTTGCGACCCCATGGGACTGCGGCACGCCAGGCTTCCCTGCC
CATCAACTCCCAGAGCTTGCTCAAACTCATGTCTGTCAAGNCGGTGATGGACTCTGTTAGAGAATTTTAAATGCTATCTCTGTGTTTAGGTGAAGCAGTACCA
CTCTGTTTTTAAGTGTTGTGTGTAGCTGGACACTGTTTCAGATTACTTGCTTCCATCTTGTCTCTCTCTCACATTTCATTATTGAAGATTATGAATATACAGA
GNAAAGAGAATAGTATCAGAACTGCCATGATTCATACCCAAGTCAAAAATAACACTACAGCTAGCTGATTATTATAATGCNTCCACACCTCACTCACTGATTT
TAAGTAATCTACATGTATTTTTNTTAATATTCATGCTGCTTAAAATAGAATCTTATTCACTAACACGACTCTTAGCTTTAAATGCTGATTCTAGTGTCAAATT
GGTTTATTTTTAATAAAAAAATTTTTTTTGCTAAAGAATTTAAGTTTTCTTCAATAAAAGTTGTTTTTTTTATTCAAA
>‘990913a2-091.scf” came from CONTIG 62 at offset 0;
“E:\SEQUENCE\export\EG_DB\990913a2\990913a2-091.scf’ (51>168)
TTTGGGGACGAGGCCAAAACGAATGCTTGGCTGCAGACAGGGTGGCCGCTTGGGCCCCCTCGGCGCGAGCCCACGTAACTGGTTTGTACCGCTTTTGTGGCCG
TGTTCTACGCGCGAC
>‘990913a2-092.scf” came from CONTIG 63 at offset 0;
“E:\SEQUENCE\export\EG_DB\990913a2\990913a2-092.scf’ (56>355)
GCACGAGGCCGGCCCCGTGCTGCTCTCACCCCAGTGGCCCTTTCCCAGGGGACCTCTCCCACCCTCCTCGAAAGAAGGACCGAAAGAACCGAAGTGGGGCGCG
GAGGGGCGACAGGCTTTGGGGTACTCCGGAGGCCCCGCCCAGCCCCTGGGGATGGGGAAAAGAAGACTCGAAACAAGAAAAGCAAGAAGCGGAAATTGAAAAA
AGCAGAACGAGGGGATAGGCTCCCACCTCCTGGGCCTCCCCGGGCACCCCCCAGCGATACAAACCTGAAGAGAAGAGGAGGAGAAGGAGGGGGA
>‘990913a2-093.scf” came from CONTIG 64 at offset 0;
“E:\SEQUENCE\export\EG_DB\990913a2\990913a2-093.scf’ (50>529)
TTTTTCCTGGGATGTGAGGCAATCTGTTGGCAAAAATTACATGTATTTGTCCTTCCTATTTGTAAGATTATATTTAATGATATTCTTTTCTTTATCAGAGACT
CTCACTGCAGGCAGNGCTATTTCTTGTGCCTAAGACTATTTCTGAAAGTTGCATCACTAATGATACTTGCCGAGTTGAGGGTGCAGAAAGTTTCTCATACCAT
ATTCAAGATAATACCAAGCACATGTGGCCGCTGGAGAGTGAAGCTGTAGTACAGAATGCGTAAATTCTGTCCCCAGGAAATCTGAGATAAGCAGGGCTGCTGG
GGTTTTTCCCCTACTCTGAGCTGTGTTCTTCTCTTTGGTAGCCTATGCTGNATAATGGATGCTAATAGTAATATTTTATGCTAGATTCAGATTTGTCCTAGTA
GTGTCTTTTTCTATGTAGAAACTTAACATGGATTTAGGGTGTGTGTTTTTTTAAAGGGAAGGAGATT
>‘990913a2-094.scf” came from CONTIG 65 at offset 0;
“E:\SEQUENCE\export\EG_DB\990913a2\990913a2-094.scf’ (51>517)
CAATTATCAAGGAGAGTTGAAAAAAGGTGATAACATCTATAATACGAGGACGAGAAAGAAAGTGCGGGTGCAGCGACTGGTTCGCATGCATGCCGACATGATG
GAGATGTTGAGGAAGGGTTTGCTGGAGACATCTGTGCATTGTTTGGCATTGACTGTGCCAGCGGAGACACATTCACAAACAAAGATAATAGCGGCCTTTCTAT
GGAGTCAATPCATGTTCCTGATCCTGTCATTTCTATAGCAAGAAACCCGTCTAACAAGAATGATACTGAAAAATTTTCAAAGTTTTTGCAGGTTACAGAGAAG
ATTCCACATTATAGTCACTTTGAGACTGCAGCAAAAGACATTGNTTTGTATGGGAGATTACACTGGAATTTTTCTCACGAAGGAAGAGATATGNTTGCTTGTT
TACAGAAAACATAGTGTTTTAAGAATTTTTGTCCCTGTCTTTTCTTTCCATAAAA
>‘990913a2-096.scf” came from CONTIG 66 at offset 0;
“E:\SEQUENCE\export\EG_DB\990913a2\990913a2-096.scf’ (58>577)
GCACGAGGGCACCACCAGCGGAAGCGCAACAACCTCATGACGCTGCCGGCGCACCGGCTGCAGCACCCCGTGCTGCTCTCCCGCCTGGTGGTCCTGGACCACC
CCCACCGCTGCAACGTCACCTACAACGGCAACAACGGCATCCAGTACGTGGCCAGCCAGGCCGAGCAGAATGCGTGGGAAGTGGGCTCCCCGCGCTCCTACTC
AGAGGCCCTGCTGGATCAAAGACCCGCCTGGTACGACCTTCCTCCGCCACCCTACTCTTAGACACTGATCTTTGAACCAAGCCGACCTGCCCCTTACCGCTCC
GGTCGTGCGTGCTGACAGCGCCGCTCCCAGCAGCCACAGCCTCTGAGCGTGACACACCACCACACCCGGGCACCTGACCGCAGAGGCCCCGTGACCCAGGACT
CGCCCCAGCAGGCATGAGACAATAACTTGCTTTTCAGTTTTTTGGTCTTTCTTTGCTTATGTCTAAACTGCGCTACGAGTTCTCGGGCCTTAGGGGGTTGAGG
GCGTT
>‘990928a-001.scf” came from CONTIG 66 at offset 0;
“E:\SEQUENCE\export\EG_db\990928a\990928a-001.scf’ (44>661)
GGACGAGGGGCAGCATCGGCCTTTGCAGGGGGGGCAGCAGCACCAGGCTCTGCAGCGGCAACCCCCCCCAGCGGCTTAAGCCATGGCGCTTTTCACGGAATTC
AGCAGCAGCCTTGCTGTAACCGACAAAGACATCTTCGAATTAAACACATTCCTCGAGTTAAACACCAAAACCTCGCAACATGGACGAGATGAACTTTCTGAGC
AGGGAGGGGTGAGGGGGGGACTTCGTGTCCCCCTTCGACCAGTGGGGTGTGGGGGCGTGGAAAAGAGCCTAAGACTACTAAATGACAACCTGGAGGGGGCCAA
GCACTTCAAACATCATGGGTTCTCCTGCGAAAGGGTAAGCAGGCTCCTCCGAATGGCTGGTGTGGATGGTTGGNCTCAAAAACAGAAAGGAGATGCTTCTCCG
GAAGATGGATGAGGAGAAAAGATTGAAGAGGTGATTGATATCTGTAGAAAGAGACTGAAACAGACAATGACCTGCCAGCCGAGAACGGGAACCCTTACCCTAA
CAGAAATACAAGAACCCCAAACAACCAATGCACTCCAAGATACACATGACAGGCCCTCCTTTTCCCTTCCCTCCGCCCTGCTCCCCTATTCTTTTTATTAGA
>‘990928a-002.scf” came from CONTIG 2 at offset 0;
“E:\SEQUENCE\export\EG_DB\990928a\990928a-002.scf’ (38>631)
CTCCAGCTCCTTTCCATCTGAAGCTGAGTGACTCTCCATTGGACTGACTGGGCAGCCTGCCTCCCCTTACCCGGGACGCCAGTACCGTCTCATTCTTGGGTAC
CAAATTTCCACTTTGCACCCCAGGTTCTCTCTCACATTCCCAAGTCCCCACTTTGAGTTCTTGTGTAGGAATCCCAAATTGCCTTTGGTAAGGGGCCCATCAC
TTACCTCCAAACTTTCTTAAGCCAATAGTCTTGCCTCTGAGCTACAAGCGCTACAGTAAGGGCCTGCTGCTCCATTTCAAGCTTCTTCAAAACTGACTCCTGC
CAGCAAAGGGCCAGAAACTCAGTTGGCTGCCCTGCTGGTCTCTGGGGGTCCATTTGTACCATAAATGCAATTTGGGAAGAAGAGGAGAAGCAAGNGGAGCCTG
ATTTCCTAATTATGGTTATCTGCTTCTTGTTCCTGTGGGAAGNTGGCTTACTGAAACAAAAGGAGCTGCTGCTTTATGTGGGCTTTAAATTATCTACTATAAA
ATTTCAAATAATACTATTTTTGTTTTATTTTAAAACTTTTAAATTTGTTTTTCTTACATAGTTAAAGAAGGAAAT
>‘990928a-003.scf” came from CONTIG 3 at offset 0;
“E:\SEQUENCE\export\EG_DB\990928a\990928a-003.scf’ (44>582)
GAACGAGGAAATCTTATGTCATGGGTAGGCGTGTAATATGGGGACACTGTGTTTAAAAGAAAAGAAAAAAACAAAACCCACATGCCACCATTTTTCTGAATCA
AATTCTACAGGGGAATGGGGGGTAAAGACTTGTACTGTGCAGGCAGCTAGATTGGGAAATGGGGGAACTAGAAGCAACGAGCACTGGAGAGCCCTCTACACTC
TTCCCCGCTGGCATCCTATTGCTTTTTGGACTATGAAGGAGAAAAGAAAAAATAGCAAGCACGTTCACATCCTTGCCCTGTTATCAACTGGGCCTGGCGCTCT
GCTAGTCATTCCCTTCCTTTTTCCTCTATCCATTTGTCAACCCTTGAGAAGCATGATTCACCTAAAATCGAGGACCGGGAACCTTGATNTGAGAGTCTGGGCT
CTCTGGCTCTTTGTGCTCCTTGCAGAGCACCACCAGCTCTGCCTCAGCCACGCTCTCTGGCAGATGGAGATCTGGCTTTTCCTGCCACCCAACCATAGCATAG
CTTAACATACACTTTATTAAAATA
>‘990928a-004.scf” came from CONTIG 4 at offset 0;
“E:\SEQUENCE\export\EG_DB\990928a\990928a-004.scf’ (34>704)
TATGTGGAAGTGATGGAAGTGGGGGTCCTGTGGGCCCTGCTGGTCCCATTGGGTCTGTCTGGCCCTCCAGGCTTCCCAGGGGCTCCTGGCCCCAAGGGTGAAA
CTCGGGACCTGTTGGTAACTCCTGGCCCTGCTGGGTCCCGCGGGGTCCCCGTGGGTGAAGTGGGGCCTCCCAGGTCCTTTCTGGCCCTGTCGGGACCTCCTGG
GAAACCCCGGAGCCAATGGGCTTTCTGGGGCTAAGGGTGCTGCLTGGCCTTCCCGGTGGTGCTGGGGCTCCCGGCCTCCCTGGACCCCGGAGTATTCCTGGCC
CTGGTGGCGCTGCTGGGCTACTGGCACCAAATGACTTGTTGGTGAGCCCGGCCCAGATGGTCGAAAGGAGAGAGCGGCAACAGGGCGAGCCGGGGCTGGGGGG
ACCCAGGCCTCTGCCCCAAGGGAAAGAAGAAATAAGCTCACTGGAAAATCGACCCGCTGCCCCCAGAACTCTGGGTGAGGGAACCTGGTCCGGGGCACTGGAC
GACGCAACTGGGCATGGCCGCGGTGCGGGGCTCGGCTGTGGGGGAGGCCATGTAATTGGGGCTGGGACTGGCTTGGACCAGTTCCGTCCTGAATTGCCATGTA
AAGTTTGGTTCTGTTTAGGAATGCCTGGCAGGAAAAAAATGATTTTCTTCCACC
>‘990928a-005.scf” came from CONTIG 5 at offset 0;
“E:\SEQUENCE\export\EG_DB\990928a\990928a-005.scf’ (12>609)
TCTACGGATCCCCCGGCCTGCGGGCTTAAGCACCAGAACAGGGGGAAATGGGCAGAATCAAAGGCAATTATGGGGCAAATAGGACCTGGAGGCTTGCCAGGCT
ATGCAGGAACAGCTGGCCAGAAACAAAAAGCTGACGCAGAAGGGCCGGGCGGGCTCTGAGAGTGAGGAAGAGGGGGAAGGACAGGAGGAGGAGGAAGAACCTC
TTGTCCCTGACCTGGGGAATGAGGTGCAGATAAAGGCAGATGGACTGAACCCCTGGATGTTCCGGAATCAATTCTTTGATGCCAAAGGAGCTGAGGGCCACAA
AGAACTGGAAGATCCTGCTGAGCCTGGAGCCCAGAAGACTTCTGAAAGAGAGGAAAAAAAAGCAGCGGGGGAGGAAGAAACTTTTTTGAAAGAATTTGAGGAA
AGGGAAACACTTACACAAAACACTGACCTAATCAATGGGAGACCTGGGCACAAACGGAACAAAGGATCGTACAACCACGCAGGGTGCCGATTGAGGCCCTGGG
CAGAAACCCCACGGAAACATAACCAGGAACAAAATGAATCACGGGAAAGACAAAGAGGAACGGGCGGGGGGAAGGGCCTTGTG
>‘990928a-006.scf” came from CONTIG 6 at offset 0;
“E:\SEQUENCE\export\EG_DB\990928a\990928a-006.scf’ (37>598)
CTTTTTTGAAAAGGAAACTGAAAGAAAGAGGGCAAGACTATTTAAGAGCTGGTAGCTGCACTGGGCAGTCTCAGCTCCTCAGCTCCCGGGCGGGACCAGTGGG
CCACCTCTGCAGGGGACTCCTGCCACCGGGCTTGTCTGGACCACTGAGCAAATCTGCCAGCACCCCAACCAGCTGTGAACATCTTCCCAGAAAATGGCAGACA
TGTCCAATGGCGAGCAGGGCTGTGGGTCCCCCCTGGAGCTGTTCCACAGCATCGCTGCTCAGGGGAGCTCGTAAGGGACCTCAAAGCCAGAAACGCAGCCAGG
ATGAAATTGATTCTGAGTGAAGATGTGTTGTCCTTAAAAAGAGCTACAAACTGCCACAGGGAGGATACAAGTGGGACTGTCCTCAGGGACCCGCGCCCGAAGG
GGGAGGCTGGAGCCACCGAGCAACAGACTTGTGATNCTGGAAAGCAACAACAGGCCAAGCATGATACACAGCTATTGTGATTGAACAGAAATGAAGAGTGNAA
ACGAAAAAGACAGACAAACAACGCTCTGCAGTATTTTTTAACAAATC
>‘990928a-007.scf” came from CONTIG 7 at offset 0;
“E:\SEQUENCE\export\EG_DB\990928a\990928a-007.scf’ (42>642)
GCACGAGGGTCAACATGACTGACAATGATCTTATCAATATTCTTGACCCTTTTTATCATCTTCAACTAAAAGTTTCAAAACACAACTTTTATCACAATCCAAA
ACTGACACCAACAAAAATATTAAAACAAAACACCCCTTGAGAAACAAAATGAACGAAAATTTATTTACCTCTTTTATTACCCCTGTAATTTTATGTCTCCCTC
TCGTAACCCTTATCGTACTATTCCCAAGCCTACTATTCCCAACATCAAACCGACTAGTAAGCAATCGCTTTGTAACCCTCCAACAATGAATACTTCAACTTGT
ATCAAAACAAATAATGAGTATCCACAATTCTAAAGGACAAACATGAACATTAATATTAATATCTCTGATCCTATTTTTGAATCAAACAACCTACTAGNCCTAT
TACCCGATCATTCACACCAACACACAATATCAATAAACTAGCATAGCCTCCCCTGGAGCAGAGCCGGATTACAGGATTCGCATAAACTAAGATCACTGTCATT
TCTACACAGNACACCACTCCTATCCATATAGANTATGAACTTAGTCTTTTTTACTAGTCTNCGGCGTACAGTAATATGAGAACTA
>‘990928a-008.scf” came from CONTIG 8 at offset 0;
“E:\SEQUENCE\export\EG_DB\990928a\990928a-008.scf’ (42>463)
GCACGAGGCTCACATTCCTGCAGACCTCCAGACTGCCCGGAGCTTGCCTGCTGCCTGCCTGCCTGCCACTGAGGGTTCCCAACACCATGAGGGCCTGGATCTT
CTTTCTCCTTTGCCTGGCCGGGAGGGCCTTGGCAGCCCCTCAACAGGAAGCCTTGCCTGATGAGACAGAAAGGGAGGAAGAAACCGGGGCCGAGGGGGCCGAG
GTACCCGCGGGAGCCAACCCCGTCCAGAGGGAAATAGGAGAATTCGATGATGGTGCCGAGGAAACCGAGGAGGAGGNGGTGGCCGAGAACCCCTGCCACAACC
ACCACTGCAAACACGGCAAGGGGTGTGAACTGNACGAGAACAACACCNCATGTGTGTGTGCCAAGACCCCACCAGCTGCCCTGCCCCCACGCCGAGGTGAGAA
GNNGGCAGCA
>‘990928a-096.scf” came from CONTIG 8 at offset 35;
“E:\SEQUENCE\export\EG_DB\990928a\990928a-096.scf’ (34>418)
CCGGGAGCTTGCCTGCTGCCTGCCTGCCTGCCACTGAGGGTTCCCAACACCATGAGGGCCTGGATCTTTTTTCTCCTTTGCCTGGCCGGGAGGGGCCTTGGCA
GCCCCTCAACAGGAAGCCTTGCCTGATGAGACAGAAGGGGAGGAAGAAACCGTGGCCGAGGGGGCCGAGGTACCCGTGGGAGCCAAGCCCGTCCAGGGGGAAG
TAGGAGAATTCGATGATGGTGCTGAGGAAACCGAAGAGGAGGNGGAGGCCGAGAACCCCTGCCACAACCACCACTGCAAACACGGCAAGGGGTGCGAACTGGA
CGAGAACAACACCCCCATGTGTGTGTGCCCAGACCCCACCAGCTGCCCTGCCCCCATCGGCGAGTTGAGAAG
>‘990928a-011.scf” came from CONTIG 9 at offset 0;
“E:\SEQUENCE\export\EG_DB\990928a\990928a-011.scf’ (36>481)
TTTTATTTACAATGCTAAGGGCTAATGTTTTTTATTTATAATACTAAATGCTAATATTCTTTGTTGTTTATTTCCCCTCAAACTTCAACCTTTAATTAAATTA
TCATCTTTAAGCTTTTGCATTTTGTTTTAATGACTGGGGTCTTAGAGCCATTGATGTCTTATGTTTGTTTTTCAAATCATATTTTTCTGCTGTTGTGGATTTG
TGTAGCTATTTGAATCATGGGCTGTTGCTTCCATTGAAGGATCTTCTGCCTTTTACTGTGGGATAACTGTTAAAATTTATGGTGAACTTTATAAGAGCTTATT
AGCATTAAAGCAGCCACCCGCAGAGAAAGGGAAACCAATCTGGAAAGCTGTGGAGTACTCATAGAATACTAATATGAGGACCAAGGACTACTAGCAACCTGAG
CCATTTCCACCTAAACGGGAACGCGTTGAG
>‘990928a-012.scf” came from CONTIG 10 at offset 0;
“E:\SEQUENCE\export\EG_DB\990928a\990928a-012.scf’ (41>541)
GCACGAGGGAAAGATTCATTGGAGATTGGGACCAGGGGTTCAAAGATTCACTGGGGTTGGTACCTTACTCTCCAAAAAGAGCAATATAGAGATGTCTCACAAA
GTTCATCCTAAACCTATTTTCTAATTTGGGAAGCTAAAAGATCACAGGCAATGTAAAACATCCATTTTTATCACTGTCATTTGTTTTCTTATTACCTCTCTCT
AGTATCTTCCACTTCTAATTATATATATATTATATTTTTTTTTTTTGGGGGGAGGGGGATAATAAGTCTTTATTTCACCATCAAATAAATTCAGGTCATGTCA
GTTTGGTAACCAAATGGATCATAAGAAAAATGTTTGCTCCATATTTTTTAAATTGTAAAAAAAAAAAAAAAAACTGAGGGGGGCCCGTACCCATTCGCCCTTA
TGTAGTGTATACATTACTGTCGTGGTTACACGTGNGACTGGAAACCTGCCGTCCCACTATCGCTGCACCTCCCCTTGCCGTGGG
>‘990928a-013.scf” came from CONTIG 11 at offset 0;
“E:\SEQUENCE\export\EG_DB\990928a\990928a-013.scf’ (40>603)
GCACGAGGCCTCACAGTGCCTGGCCAAGCCCTGGCCTGTGGGAGCAACAGGGTCAACAAGCTGGGCCTGGACCACCTCTCCCTAGGGGAGGAGCCTGCCCTAC
ACCAGCAAGGGGAGGAGGCCCTGAACTTCACTCCATTAGGTTCTGCACCCCTGGACCAGGAGCCCGTGCTGAGTGTGGATTTGGGCACTGCTGATTCAGCTGG
TGTAACTGCCTCTGGTGACTGCTACACTTTCGGCAGGAATCAGCATGGGCAGTTTGGGTACCAATGCTCGCCGGGTCAGCCGGGCAGCCTGTCAAGTCCAGGG
GCTCCAGGGCATCAAGATAGCGATGGTGGGCTGGGGGATGCCTTCACTGTTGCCATTGGGCAGAAGGGAAGGTACTCCTGGGCAAAGGGCCCGAGTNCGCTGG
GAGAGAGAGNAGACGCCGACTCCTAGGCAGGCAGTGGAGAGACCACCCTACCATGACCTTGTGCCTGTGCCTGCACACTTCTGCTTCGCCTTACGAGAACATG
CCCCTGCCACTGATCATCTGTACCTGATGTTTCTGGAGTTGAAGGAGGCA
>‘990928a-014.scf” came from CONTIG 12 at offset 0;
“E:\SEQUENCE\export\EG_DB\990928a\990928a-014.scf’ (41>599)
GCACGAGGAAGAATCTCTCTTTAGTTCTTTGTACGGAGGTGAAAAAAATAAAAAAATCCTTCAAAAATGGCAATGGTATCTGAATTCCTCAAGCAGGCCTGGT
TTATTGAAAATGAAAAGCAGGAATACATTAAAACGGTGAAAGGATCCAAAGGTGGTCCTGGGTCAGCAGTGAGCCCCTATCCTACGTTCAATCCATCCTCGGA
TGTTGAGGCCTTGCACAAAGCAATCACAGTTAAAGGTGTGGATGAAGCAACCATCATTGAAATTCTGACTAAAAGAAACAATGCACAGCGTCAGCAGATCAAA
GCGGCCTATTGTGGAGAGAAAGGAAAGCCCCTGATGAAGTTCTGATAAAGCCCTCCTCGTCACCTTGAGAGTTGTTTTGCTTCTTTGAAACTCAGCCAGTTGA
GCCGAGACTCCTGCTGCCTGAAGGCCTGGACGATGAGACCTTGATGAATCTGCATCAGACTACANNAAATCAAAATACAAACTTANAAAACTAGAAAAATGCT
AAGACTGCTCAACCTTGAATATAAGTTTGTGTTGTTGTGCCGTC
>‘990928a-015.scf” came from CONTIG 13 at offset 0;
“E:\SEQUENCE\export\EG_DB\990928a\990928a-015.scf’ (40>545)
GCACGAGGGCCTAACACAAATTAAGAATATGTTTCAGTTTATATCATATTTCAAAACACATTTAACTGTTTCTTAAAGCTTTACATTTTTAGTTACAACCTCC
AGAGATTTTGAGCCTCATTTTCTTCAATACTTGAAATAGAGGGAGCTAGAACACTTCATCATGTGTAATCTGATAAACCTGCTGCAAGAGCCATAATTTTGAG
GACTTTCTAAGGGAATTGTGGGGATCCAGGATTTATAATTTCTTGATCTAAACTTGCATAAAGGAAATACCACACGTGCACATTTCATAGTATGAAATAAGAA
GTCATTTTTACACTTTATTATGTAAGTAATATATGCACCTTTCATAATCCATGTGTGTTCACTAAGCATGTTATATAATATGATTGACAGATTTGTTTTTTTC
TANAATAAATATTTGTGGGGTTTGTTTTGTTACAATATCATTTTGCTATTAACATGCAATAGACGGATGTGCCTTTTATAAGATAGTTTCGGG
>‘990928a-088.scf” came from CONTIG 14 at offset 0;
“E:\SEQUENCE\export\EG_DB\990928a\990928a-088.scf’ (36>595)
CGAGATGGTTCTCCTGGCGCCAAGGGTGACCGTGGGGAGACCGGCCCTGCTGGGACCTCCTGGTGGCTCCTGGGCGCTCCCGGGTGCCCCCGGGCCCTGTCGG
GACCTGCCGGGCAAGAGGCGGTGGATCGTGGGTGAGACCGGGTCCTGCTGGGTCCTGCTGGGTCCCATTGGGCCCCGTTGGGTGCCCGTGGGCCCCGCTGGGA
CCCCAAGGGCCCCCGTGGTGACAAGGGTGAGACAGGCGAACAGGGCGACAGAGGCATTAAGGGGCACCGTGGCTTCTCTGGTCTCCAGGGTCCCCCCGGCCCT
CCCGGCTCTCCTGGGGAGCAAGGGTCCTTTCCGAGCCTCTGGTCCTGCTGGTCCNCGCGGTCCCCCTGGCTTGCTGGTTCTCCCGCAAAAATGACTAATGGTC
TCCCAGCCCCATCGTCCCCTGGGCTGNAGNNGAACTGGGAGGTGGTCTGCTGGTCTCCCGGCCTCTGACCCCTGNCCCCAGCCTCCACGAGATACATTGACTA
CTGCCCACCACCTAAAAAGATAGAGTGTCGGTATCGGCTTGTGCA
>‘990928a-017.scf” came from CONTIG 14 at offset 259;
“E:\SEQUENCE\export\EG_DB\990928a\990928a-017.scf’ (40>593)
GCACGAGGGGCACCGTGGGTTTTCTGGTCTCCAGGGGCCCCCCGGCCCTCCCGGCTCTCCTGGGGAGCAAGGTCCTTTCGGAGCCTCTGGTCCTGCTGGGTCC
CCGCGGGGCCCCCTGGGTTTGCTGGTTCTCCCGGCAAAGATGGACTCAATGGTCTCCCACGCCCCATCGGTCCCCCTGGGCCTCGAGGTCGCACTGGTGATGC
TGGTCCTGCTGGTCCTCCCGGCCCTCCTGGACCCCCTGGTCCCCCACATCCTCCCAGCGGAGGGTACGACTTGAGC1TCCTGCCCCAGCCACCTCAATAGAAA
GATCACGATGGTGGACGCTACTACCGAGCTGATGATGCCATGTGGGGCGTGACCGGGACTCGAGAGGACACCACCTCAAGACCTGAGCAGAAATCGAGAAATN
CGAACCTGNAGCAGACGCAGACCCCGCCGACCTGCGGACTCAAATTGCACTTGATGGAGAAGGAGATAATGATGACCCACCAGCTGCACTGATGCATAAGTCT
TGGAATGGAACGGGAGACTGGATACCCCTATCCACGGAC
>‘990928a-018.scf” came from CONTIG 15 at offset 0;
“E:\SEQUENCE\export\EG_DB\990928a\990928a-018.scf’ (34>577)
CGTTCGACAGATAGCCGGAACTTCTGTGCTGTGCCAGCGGCATCCCTGAGAAAAAGGGGAAGGTCGGAGTGAACGGATT7CGGCCGCATCGGGCGCCTGGGCA
CCAGGCTGTTTTTAATTCTGGCAAAGGGGACATCGTCGCGATCAATGACCCCTTCTTTGTCCTTCACTACATGGTCTACATGTTCCAGTATGATTCCACCCAC
GGCAAGTTCAACGGCACAGTCAAGGCAGAGAACGGGAAGCTCGTCATCAATGGAAAGGCCATGACCATCTTCCAGGAGCGAGATCCTGCCAACATCAATGGGG
TGATGCTGGTGCTGAGATGTGGGGGAGGCACTGNNGTTCTCACTACCATGGAGAGGCTGTGCTCACTTGAGGGGGAGCCAGAGGTCATCATCTTGCCGTTTGC
CAGCCCCCTGTTGGGAGGGGGGAACAGANAANATACACACTCAAATGCACATGGTCCGCACACACGCTGCCCCCGCCAGCATCTACACTTGCATGGAGACTAT
ACATTCAGCCTCCTCACAAAATGGAGGCCC
>‘990928a-019.scf” came from CONTIG 16 at offset 0;
“E:\SEQUENCE\export\EG_DB\990928a\990928a-019.scf’ (34>624)
CGGAAACCACGTACCGGCAAACTGGCCTGGCTCCCGGGCAGGAGTATGATATATCCTTGCACATTGTGAAAAACAATACCCGGGGCCCTGGCCTGAAGAGAGA
GACAACTACCCGCTTGGACGCCCCCAGCCAGATTGAAGGGAAAGATGTCACAGACACCACCGCTTTGATCACTTGGTCCAAGCCCCTGGCCGAGATCGATAGC
ATTGAGCTCATGTACGGGATCAAAGACATGCCAGGAGATCGTACCACCATCGATCTCACACACGAAGAGAACCATTACTCCATTGAGAACTTGAAGCCGGACA
CCGAGTACGAGGTGGCCCTCATCTCCCGCAGGGCCGACATGTCCAGCAACCCCGACAAAGAGAACTTCACAAACAGCCTGCATGCGGCCAGAAATCTCCGCCG
CATCTCCCAAACAGACACAGCATCACCTGGAGTGGAGGAACGTCAGGCAGCCGCCGACAGTAAGAATTAATATGCTCCATCTTGGAGCGACACGCGGAGAGAA
GAGCCAGAACAAACACACAACAAACACCTACAGGCTGAGGCAGAAGATTTGGTTGGGGGTGGTGAAGAGAAGGAAG
>‘990928a-020.scf” came from CONTIG 17 at offset 0;
“E:\SEQUENCE\export\EG_DB\990928a\990928a-020.scf’ (35>533)
CTGAAATTTAAGGAATATAATCTATTGCTTTATTTTGGCACTGGTTTGCAGCATGTGATGTTCATTTTCTGTGTACATGATATGTCCTCATCTGGATGCTAAA
CTCAGGAAATCAGAGAAATGCATATGCGTTTTCTATGTTGATTTCTCCTTTTCTTAATCATGCTTGTCTTAAAAAAAATATTATCTAAAATTTACTGGGAACT
GGATTCATAGCTGGCACCTTGTTTTTATGTAAACAGATTGCAGATAGACCAAAGATCATCTGGACCATTTTGTGGTTCCCTGCCTGTCTTCTGATTATCGCTC
TCTCATCCATCCTTTCTCTTCTAGAAATTTTCCTTTTCTGGTCTTCTCCTTATGCTTTCTTTGATCTCTGACTTTCAGTGACTTANGGGAATAGTTACATTCC
CCTGGATTANCTGNTAGCTACCTCCTGCATCCCTTANGGATTTTTATCTTAATTCTAATTTGTCTTCAGTTANATCCTTCTCCAATGT
>‘990928a-023.scf” came from CONTIG 18 at offset 0;
“E:\SEQUENCE\export\EG_DB\990928a\990928a-023.scf’ (35>601)
CGCCACTGAAGATCCTGGTGTCGCCATGGGCCGCCGCCCCGCCCGGGGTTAGCGGGATTGTAAGAACAAGCCGCACCCAAAGTCTCGCTTCTGTCGAGGGGGC
CCTGATGCTAAGATTCGCATCTTTGACTTGGGGCGTAAGAAGGCCAAAGTGGATGAGTTCCCACTCTGTGGCCACATGGGGTGAGATGAGTATGAGCAGCTCT
CCTCTGAAGCCCTGGAGGCTGCCCGTATTTGTGCCAACAAGTACATGGTGAAAAGATGTGGCAAAGATGGTTTTCACATCCGAGTGCGGCTCCACCCCTTCCA
TGTCATCCGCATCAACAAGATGTTGTGTTGTGCTGGAGCTGATAGACTCCAGACAGGTATGCGCGGTGCCTTTGTAAAGCCCCAGGCACAGTNGGCCAGGTCC
ACTTGNCCCAGTCATATGTCCATCCGCACCAGCTGCAGACAAGGACATGGATTGAGCCTCCGCGGCCAGTTCAGTTCCTGCCGCACAAATCAATCTTATAATG
AGATTACAAGTCACCGAGATTGAGAATGAGAGAAAGGATATCAACGCTGGGG
>‘990928a-051.scf” came from CONTIG 18 at offset 0;
“E:\SEQUENCE\export\EG_DB\990928a\990928a-051.scf’ (33>601)
CGCCACTGAAGATCCTGGTGTCGCCATGGGCCGCCGCCCCGCCCGGGGTTACCGGTATTGTAAGAACAAGCCGTACCCAAAGTCTCGCTTCTGTCGAGGTGTC
CCTGATGCTAAGATTCGCATCTTTGACTTGGGGCGTAAGAAGGCCAAAGTGGATGAGTTCCCACTCTGTGGCCACATGGTGTCAGATGAGTATGAGCAGTTCT
CCTCTGAAGCCCTGGAGGCTGCCCGTATTTGTGCCAACAAGTACATGGTGAAAAGCTGGGCAAAGATGGTTTTCACATCCGAGTGCGGCTCCACCCCTTCCAT
GTCATCCGCATCAACAAGATGTTGTGTTGTGCTGAAGCTGATAGACTCCAGACAGGTATGCGCGGTGCCTTTGGAAAGCACCAGGGCACAGTGGCCAAGGTCC
ACATTGTCCAGTCATTAGTCCATCCGCACCAGCTGCAGAACAGGAAATGTGATGAAGCCTGCGCGGCCAGTTCAAGTCCTGCCGGACAAATCACATTTAAATT
GGGATTACAAGTCAAGCGAGAATGGAAAGTGGAGAAAAGATATCCAAGGTGGGG
>‘990928a-024.scf” came from CONTIG 19 at offset 0;
“E:\SEQUENCE\export\EG_DB\990928a\990928a-024.scf’ (27>564)
GTGGTGGTGCTGTCTCAGGGGCTGAAGCCCATAAGGCCCTCAGGAAAGCAGAAAGCTTGTATATATCTCTTTGTGTCGTGGAAGCCAAAGTGAAGGCCCAGGC
CGCGCCGAACAGGGACGTGCAGAGAGAGATTGCGGACCTCGGTGAGGCCTTGGCCACTGCCTTCATCCCTCANTGGCAAAATGATGAATTACGGGAGAATTTT
AAGTATCTGAATAAGGTCATGGATGACCTATACCGGGCTAGCAAAGCCGATGTGCAGAATCGGGTGTTGGAGAAGACTAATCAACTCATCGACAGCAGCCCCA
ACCAGCCCCTGGTCATGCTGGGAGTGGAGAGCGGCGCCTCGGCCAAGGCCTGATCGAAGCCTGTACTCTTTANATGCTTCCCCCCACACGCCGTCTGCTCTTT
ACATGGATACGAGCCGCAGATACTGCTTGGTCAGTACCCACATGCAGCACGGGGCTGAGGCATGAGGGGGAGAGGGTCAGCTGTGATGCAAGAGAGGAAACGG
CTGTAGCCAGCAGAGGGCTCTGT
>‘990928a-026.scf” came from CONTIG 20 at offset 0;
“E:\SEQUENCE\export\EG_DB\990928a\990928a-026.scf’ (34>601)
GGAGCGAGCGCCATGGTGCTGCTGCACGTGCTTTTCGAGCACGCGGGCGGCTACGCGCTTCTGGCGCTGAAGGAGGAGGAGGAGATCAGCCTTCTGCTGCCGC
AGGTGGAGGAGGGCGTGCTGAACCTGGGCAAATTCCACAACATCTTCGTCTTGTGGCCTTTTGTCCCTTTTCCTCGTCCCAAGTTGCCTTGGAAAATGCCAAC
GCTGTGTCTGAAGGTGTTGTTCATGAGGACCTCGGCCTGCTCTTGGAGACTCACCTGCCACCCAAAAAGAAGAAAGTACTTCTGGGAGTFEGGGGACCCCAAG
ATAGGTGCGGCTATACAAGAGGAGTTAGGGTACAACTGCCAGACTGGAGGTGTCATAGCCGAGATCCTTCGAGGAGTTCGTCTGCACTTCCACAACCTGGTGA
AAGGTCTGACCGATTTGTCTGGCTGTAAAGCCCAACTGGGCTGGGACACAGCTATTCTGTGCGAAAGTTAAGTTATGTGAACCGATGGACACATGATTATCCA
GTCTATANCCTCCTGGACACTGGAAAAGATATCATACTTCTTCTGCGTGTAGG
>‘990928a-027.scf” came from CONTIG 21 at offset 0;
“E:\SEQUENCE\export\EG_DB\990928a\990928a-027.scf’ (33>427)
CTTAATTCAAAGAGGGGTGGAAACGTGGAGCAGCGGAAGTCTTCCTGGGCGCCTAGATTCCCGGGAGGGAAGGAGGAGCTGACTGGGAATAATTTTTTCTCCT
CACCCCCCACGCCCCCGGATATCGCCCTCGGCACTCACCATGAAGAAAAATGAATAGAGGGGGGGCACATGGATTTCCAGTAGCCGCCGAGTGTGGGAACTCC
CAGGTTTGCTTTTTATGGTTTTCTGGTGAAAAGCGGCATGTGAACAAATCCATGAACAAACCCAAGAGCTGTGAATATTCGCACAGAAGGTGCTGGGGCGGAG
TGGGAGGCGGCGGCAAGTAGCCTTCAGGTGGGAGGGAGAGAGAAGATCGCCCCACTGACCTTGAAGACCTCCCTGTAAGTCCCACG
>‘990928a-029.scf” came from CONTIG 22 at offset 0;
“E:\SEQUENCE\export\EG_DB\990928a\990928a-029.scf’ (39>424)
GCACGAGGCTCTTTCTGTAAAAGGTGCAGAGGTGGCATCAATGACGATGAATGTGATCCACACTGTGCCTAACTTGGATTGGCTTTCTGTGTGGATCAAAGCC
TATGCTTTTGTGCATGTTGGTGACAATTCGAGAGCAATCAATACCATTTGTTCACTANAGAAAAAGTCCTTGTTGAGAGATAACGCGGGACCTACTGGGAAGC
TTAGCAGATCTGTACTTCAGAGCTGGAGACAATAAGAACTCTGTCCTCAAGTTTGAACAGCACAGAGTTTGGATCCATATCTAATAAAGGAATGATGTGTATG
GCTATCTCCCTGCAGAGAAGACGATGGAGNAGNNGGAGACCTTGCTGCCGCCTTTTATATATCTGACCACAGCACACC
>‘990928a-030.scf” came from CONTIG 23 at offset 0;
“E:\SEQUENCE\export\EG_DB\990928a\990928a-030.scf’ (39>593)
GCACGAGGCGGAGACGCGAGGAGCAGGAGGCTCGGGAGAAGGCGCAGGCCGAGCAGAGGAGCAGGAGCGGCTGCAGAAGCAAAAAGAGGAGGCCGAAGCTCGG
TGCCCGAGAAGAAGCAGAGCGGGAGCGTCTGGAGCGGGAAAAGCACTTCCAGCGGGAGGAGCAGGAGCGGCAAGAGCGTATAAAGCGCTTGGAGGAGATTATG
AAGAGGACTCGGAAGTCAGAAACTGCTGAAACCAACAAGCAGGACAGAAGGAGGCGACGGCCAACAATTCCAGCCCACGGATAGACCCTGCGAAAGCTGTGGA
GGGTCGGCCTGCCGGGGTGCAGATGAGGAGCTGGGCCCCCAGAGCCTCATGGGAGCCTGCCAACAAGAAGTCGCTGGGTCCCTGGTGATGGGCTGCAGCTTGC
CACGCACCAGATAACGGTTNTCTCTATGGACCCTCNGACAAGAGTGGCNCGACGCACAGCTCTCTGCCTTCCAAGCATAACTATTATAAATTGGTGGAGCCCT
AGCCATAATCTTAGGTTGCTGTTCAGCGAGTATAGCTCTT
>‘990928a-031.scf” came from CONTIG 24 at offset 0;
“E:\SEQUENCE\export\EG_DB\990928a\990928a-031.scf’ (34>418)
CAGAGATGGAGGCAAACATGAAGGGAAAGACATGTAATGAAGATTTTTTTTTTTTTTAATCGTGGTCTTTTTTTTTTTTCCAAAGCAATGTTTAAACTAATAA
ATATTTTTTTATGAAGGGGAAAGCGGGAAAATCACATCTCACATTTTTGTACCTTCCCGTGTATGCGTCGGGGTTCGGCGTCTGCATCACCAACGCGGAAGGT
GGGCCATCAGGCGGCTCGTTCTTTGGGGTAGGGGAGGGGCGGAGAACCAAGATTTTGCATCAAGCTCTGGATCCTGACGGGGTTGTATGTTGGGACTCTGAGT
TGGGGAATAGTCACAACACTGCCAGAACTGGATAGGGACCTCGATTGAAAGGACACAGCTACTGCTTCCACTT
>‘990928a-032.scf” came from CONTIG 25 at offset 0;
“E:\SEQUENCE\export\EG_DB\990928a\990928a-032.scf’ (39>446)
GCACGAGGCCCAGGGGGGAAGGCGTGGAGGCTCCTGTGGTAGCCCTCCTGAGCTGTGCTGGAAGCCTGGACTGACCAATCACCCAATCCCAGTTCTTCGTGCA
AAACTCTGTGGCCAGCCTGGCATCAGTTCAGGCCTCTGCTGGGGGGAGGGGGGCCAGGCCTGGTTGTCTAAATGCAGGCAGCTGGGAGAGGGGGGGTGGGTAT
GTTTCCATGGTTACCATGGGTGTGGAGAGAAGTTGGGGCCCCTACTCTCCCAGCTGAGCGGCCCTGCGGCTTGAGTGGATGCATTGATGCGTNTCTGTCCTGA
GGCTCATCTGTGTGTGTGTTTGTGGTTTAAAATACTATGTTTGCCCCTCCTCACAAAAAATGCACTTGTATATTCCCTTTAGTGAAAATGGTAAGGGAT
>‘990928a-035.scf” came from CONTIG 26 at offset 0;
“E:\SEQUENCE\export\EG_DB\990928a\990928a-035.scf’ (39>316)
GCACGAGGCTTAACCCACTGGGTTTATAAGAAGTATCCAAACGTGTTTAAGAAGATGCGAGGCATTGTGGAAGAGAGCGTGACTGGGGTTCACAGGCTGTATC
AGCTCTCCAAAGCTGGGAAGCTCTGTGTTCCGGCCATGAACGTCAATGATTTTGTTACGAAACAGAAGTTTGATAACTTATACTGCTGCGGAGAATCCATTTT
GGATGGCCTGAAGAGGACCACAGATGTAATGTTTGGTGGGAAACAAGGGTAGTGTGTGGGTTGTGGAAGGGG
>‘990928a-036.scf” came from CONTIG 27 at offset 0;
“E:\SEQUENCE\export\EG_DB\990928a\990928a-036.scf’ (39>557)
GCACGAGGGCGCACTGCGCCCGGCCCAGCTTCTTTCAAAATGTCTACCGTTCATGAAATTCTGTGCAAGCTCAGTTTGGAGGGTGATCACTCCACACCTCCAA
TGCATACGGGTCAGTCAAAGCGTACACTAACTTTGATGCTGAGCGGGATGCTCTGAACATTGAAACAGACATCAAGACCAAAGGTGTGGATGAGGTCACCATC
GTCAACATCCTGACCAACCGCAGCAATGAACAGAGACAGGATATTGCCTTCGCCTACCACAAAAGGACCAAGAAGGAACTTGCATCAGCACTGAAGTCAGGCT
TGTCTGGCCACCTGAGACAGTGATTNTGGNCCTATTGTAAATACTGCTCATATGATGCTTCTGACTGAAAGCGTCATAAGGGGCTGGGACTGATGAGACTTCT
ATTGGAACATCTGCTCAGGAGAACCAGACTGCAGAATCACAGAGTTTAAGAAAGTACAAAGGATTGGAAAGAATTGNTTCGAACATTGGGATTCGCAGTGAGG
CGCC
>‘990928a-037.scf” came from CONTIG 28 at offset 0;
“E:\SEQUENCE\export\EG_DB\990928a\990928a-037.scf’ (39>565)
GCACGAGGATATTCCATTTTGCATTTTCCCCCTACCCCCAAGAAAAAGAAATGCAAAAAGGCAAAATGGTTGTTTGAGGAGGTTTTACAAATAGATGAGAAAA
GAAGAGAAGCGAAAAGCAAAGGAGAAAAGGAAAGATACATCCATCTGAATGCAGAGTTCCAAACAATATCAAGCAGAGATTTAAAAAAGCCTTCCTAAGTGAA
CAATGCCAACAAAGGTCCATCTATTCAAAGCTGTGGTTTTTCCAGTAGTCGGGTATGGATGTGAGAGTTGGAGTATAAAGAAAGCTGAGCGCCAAAGAATTGA
TGCTTTTGAACTGTGGTGTTGTTGAAGACTCTTGTNATCCCTTGGACTGCNAGGAATCACATCATATCATTCTAAGNAAANACTGCTGCATATTCCTGGAGGA
CTGAGTTGAAGCTGAACTCCATACTTTGCCTCTGATGCAACACTGACTCATGAAGACGTGATGTGGGAGATGCAGNAGAGAGAGGGAGAAGAGATGAAGGTGG
ATGCTCCTGCTC
>‘990928a-053.scf” came from CONTIG 28 at offset 0;
“E:\SEQUENCE\export\EG_DB\990928a\990928a-053.scf’ (39>582)
GCACGAGGATATTCCATTTTGCATTTTCCCCCTACCCCCAAAAAAAAGAAATGCAAAAAGGCAAAATGGTTGTTTGAGGAGGTTTTACAAATAGATGAGAAAA
GAAGAGAAGCGAAAAGCAAAGGAGAAAAGGAAAGATACATCCATCTGAATGCAGAGTTCCAAACAATATCAAGCAGAGATTTAAAAAAGCCTTCCTAAGTGAA
CAATGCCAACAAAGGTCCATCTAGTCAAAGCTGTGGTTTTTCCAGTAGTCGGGTATGGATGTGAGATTGGAGTATATAGAAAGCTGAGCGCCAAAGATTGATG
CTTTTGACTGTGNGNTGTTGAAGACTCTGAGAGTCCCTTGACTGCCAGAATCAATCATTCTTCTAAAGAATCAGTCTGATATTCCTGGAAGACGAGTTGAAGC
GAACTCATACTTGCCTCTGAGCAAGACTGATCATTGAAAACTGATGTGGAGATGCAGAGAGAGAGGAGACGAGACAAGGTGATGCTCCTGCTCCTGAGATTGG
AATCAGGTGGAGGACGGAGGCTGTGGGCG
>‘990928a-038.scf” came from CONTIG 29 at offset 0;
“E:\SEQUENCE\export\EG_DB\990928a\990928a-038.scf’ (33>524)
TGGATGTGCAAAATTATTCTTCCATACTGAGTATGAGAAAACAACTCCCAGAGGAAACCTTATGGATATAAAGAGGGTGGGAAGACCCTCAGGCAAAAGAAGG
TCTTAGTCTACATGAGAGAATTAAAAATGGAGAGAAACCCTTTGAATGTACTGCCTGTAGGAAAACTTTCAGCAAGAAGTCACACCTCATTGTACACTGGAGA
ACTCATACAGGAGAGAAACCCTTTGGATGTACAGAATGTGGAAAAGCTTTTAGCCAAAAATCTCAGCTCA7FFATACACCTGAGAACTCATACAGGAGAGCGA
CCCTTTGAGTGTCCAGAAGTGGGAAAAGCTTTCAGAGAAAAATCTACTGTCATTATACATTACAGGACTCATACAGCAGAGAAACCTATGAATGTATGGAAGN
NGGAAAGCCTTCACTCAAAGTCAAACTCATGTCCATCAGAAACCCCACAGAGAAAAACTATGATGCCGAATGGGGGATCTTT
>‘990928a-039.scf” came from CONTIG 30 at offset 0;
“E:\SEQUENCE\export\EG_DB\990928a\990928a-039.scf’ (33>543)
CGGCGGCGGCTCCCGTCGGCCTTCTGCAAGAGTGACCGTAGGGAGGCCGGAAAGCGGGCGCGCGCGGCGGACAGGCGGGCGGGAGGCCGGAGCTTATTGAATG
AAAACTGCCAGAGACTGAAAAACCAAACATGAGGGGAGCAGGTGCTGCAAAGTTGGGGGTCGCTGTGGCAGTATTTTTACTGACATTCTATGTTATTTCTCAA
GTATTTCGAAATAAAAATGGATGCAAGTTTAAGAAATCTATTTGCAAGGTCAGCATTGGATGCGGTTGTACGTTCTACAAAACCTCCCAGATATAAATGTGGA
ATCTCAAAATCTTGCCCTGAGAAGCATTTTGCTTTTAAAATGGCAGTGGAGCAGCCATGTGGGGGGACCCAAATCTGCCTAGAGACATGTTTTATGAGTGGTG
TAAAAATAGTTGAAANAGAATCAAGNTGCCTGGTAATGGCAAAACGAAGACTAAAACACCAATTTTGACTGGGGGGGNAAGGGCCCTTTTTATTCTAAGC
>‘990928a-041.scf” came from CONTIG 31 at offset 0;
“E:\SEQUENCE\export\EG_DB\990928a\990928a-041.scf’ (39>575)
GCACGAGGCGGGCGTTCTGAGCATACTTTATTTCCGCTCTACTTGCTGCCTGCTGCTTACTCATCCTGATAGTACCACTATCTTTAAACTCCTAACATATAAA
GGGATAAACAAAAATGATTTGTGGGGATGGAGAGAAGAATAGGATGATGGCACTTAGTAAACTCGATGTATACCCAGTATATCATTTATCATTTGCTGGGTAA
CAAATAACCCCCTCAAATGTAGTGGCTTCAACACCATTTATTTATCTCATGATTCAAAGTCAGTAGTTTGAGTTGAGTTCAGTTGGGCGGTTTTTCTGCTCGT
GGTTGGGCTCACTCTATGGTCAGTTGCTAGGCAGGGGGATGGCTAGTGTCTAGAGTAGACTTAACCAAATGACTATCTCTGTTTCACATGTTCTGTCTATATT
CACTAGCCACCTGGGCTGTTGACTATAGCTGCATGATACAGGAGGATAGAGGACCCCACCTATCCTCTCTGAGCCTGCCCTGCGATGAGCTAGAGTTTTTTTG
ATAACAATAGAAGGAAACTGA
>‘990928a-042.scf” came from CONTIG 32 at offset 0;
“E:\SEQUENCE\export\EG_DB\990928a\990928a-042.scf’ (39>470)
GCACGAGGATCCATGGGCACTATGCAAAAATCGGCACCGTCCTGACGTTCTTTTGTAACAGCTCCTATGTTCTCAGTGGTAATGAGATGAGAACTTGCCAGCA
AAAGGAGAGTGGTCAGGGAAACAGCCAATCTGCATAAAAGCCTGCCGAGAACGAAAAATCTCAGAGCTGGTGAGACGGAAAGTTCTTCCAATGCAGGTTCAGT
CAAGGGAGACGCCATTACATCAGCTATACTCATCAACCTTGAACAAGCAGAAACTGCAGTATGCCCCTACCAAGAATCCAGTCCTTCCCTTTGTAGACCTGCC
GCCTGNGTACCAACATCTGCACACCCACTCATACGAGTGCATCTCACCTTTATCNGCGCTGGCAGCACCGGAGACGGTCTAAGATGNGAGGGAGGGGCGGGCC
CATCTGCTNCTATTGTGGAA
>‘990928a-044.scf” came from CONTIG 33 at offset 0;
“E:\SEQUENCE\export\EG_DB\990928a\990928a-044.scf’ (39>566)
GCACGAGGGGAAGCCCTCTTCTTTGTTGGTTATCCATCTTAAATGTAGCAGTGTGCACATGTCAGTCCCAAACTCCCTAACTATCCCTCCCCCAAATCAGAAT
TTTAGTTAGGAATGGGAATTCCTGTAGTTACATAGAAACTTAGAGAGAGGCAGCTTGTTGAAGGTTATAGAACTATGCAGCTAGTCACAGAGCTAAAACCACA
GAAAGGTATCTGTATTCCTTTTTCCTGTTTATACTCTATCAGATTGCATATTTACCTTATGGAGGAAAAAAACAGATTTAAGAAATCTGTTTATGGCACCAAT
ATTTTTTCTTTCAAGCTTTGTCTTTCCAGCACCAAACGTTAGATGTAATACACAAGCACACATACTGTGTGTGTGGGTGTGTGCATTTGTGCATGTGGATTTT
ACAAAAGTTNTTATTGAAAAGCAAGAAATATATAGCACTGATACCTCTTTCTGGACACAGATACTGGTTTCTTAACATATCACCTCCCCTCTCCAAAAAAATT
TCAGCAGNAAAC
>‘990928a-046.scf” came from CONTIG 34 at offset 0;
“E:\SEQUENCE\export\EG_DB\990928a\990928a-046.scf’ (39>526)
GCACGAGGCTCTCCTTACCTAAGTCCAGATTTGTTTTTAAGGCATAGAATGTATAGCAACCCACCCAGTTCCAATCTGACTGATTTTTCCAGACAAGAAACAA
GCAAACTGACCATGGATGGCTGGAGGGCATCGACACCGACTGCAGGATACACGATGCCGCAAACAGGCAGCAGCCTCTTCATAGGAGTGTCCAGCTACTCCCT
GGCTTCCCCACTGTTGTATAAAGAAGGATTTGGCTGGTCTTTGTTGCCACTTCCTAAGAGGAACCTATAAACTTTCATAACTTCCTTGGTGATAGGAGTGTCT
TTGTTATTCATGGTGGACCCTTTGGATCTCAACTTTTTACTTAACGAGGTGACTCATGGTGGGCCCTANATAGTTTCAGGATGGGCTGGGTGTGGCTGAGAGA
TCAACCATGAGATACAAAGCTGGGCCCTGAGTATCTGCTTCTGACATCTGGGTGGGAGAGAGAAAGGGAAATGAGN
>‘990928a-047.scf” came from CONTIG 35 at offset 0;
“E:\SEQUENCE\export\EG_DB\990928a\990928a-047.scf’ (33>617)
AATTCGTGACGAGGAGAGAACTAGTCTCGTGTTTTTTTTTTTTTTTTTTTTTTTTTTTTTTTTTTTTTTGTTTATTTTTTCAATTTTTAAAAAACAAATTACC
CCAACAAAACACAAAACTCCCCATCCCAAAGCAGATAAGCTTAACACGTTAGGGTTAACAGGAGAGAAAGGGGTTAAGTCACACACGACTTGGGGGTTGGGGG
GTGACGCGGCCGGAACCCGGGGGGGGGCACGGTTGCTTATCGCCCCAAATAAATAAACACGGGGCCCTCCCCTCAGGGAGAGGAGGGGGAAGGGAAGCACGAC
GGCTGGACAAACATGCGCGTTATGCCAAAACGTGGCTTAAAAAAAAAGAAAACCCACAAGCAGGAACGCTCAGGACACCTGTGAGTAGGGTGGGCAGGAGGGG
ATCGGCCAAAAGCCAAACCACCGAACCCGGGGGCCTGGGGCCCCACCTGCCTCACAGATGCGGGACCCCACCCTGGCTTTCGAATAAGGAGGTAGGGGGACAC
ACCCACCCTAGGCCATCCCAGGAGGCCCAGCTTGCAGCCCCGCCCCGGGTCCCCGCCATTGGAGCGGGGG
>‘990928a-049.scf” came from CONTIG 36 at offset 0;
“E:\SEQUENCE\export\EG_DB\990928a\990928a-049.scf’ (26>628)
CTGTTTAATTCGGCACGAGGCTCAGCTGCAGCCCGCACCGTTGCCGCCTGGTACCGGGTTGGGAACAGGAGCCTAGGGGTGCTTGCAGCAGGCCGTCCCGGGC
AGCAAGGTGGGGGGCCCTACAGCGATGCTCCGAGGCATGTACCTCACTCGGAATGGGAACCTCCAGAGGCGGCACACCATGAAGGAAGGCAAGGACATGAAGA
ACAAGCTGGGCATCTTTAGGCGGCGGAACGAATCGCCTGGGGCCCAGCCAAGGGGCAAGACAGACAAAGTGATGAAGTCATTCAAGCCCACTTCAGATGAAGC
GCTCAAGTGGGCGAGTCCTTTGAGAGCTACTGGTCCACAATAGGGTCTAGCGGTGTTCCATGCCTTCCTCCGACTGAGTTTATGAGGAGAACTGGATTCTGCT
GGATGCGAGACTTCAGAAGNNCAGCACAGCCAGATGCGCCAAGCANAAAACTTGCTGGACATCGCATCAGCGGCAGGAGAAACTGACTGNAACAGGACAACAA
GACACTANANGTACTCGGCTGTTGACTCACAATGCATTGGTATGTGAGATCTACCCCTCTCCTGCCTTCTGACTTACAAAAAAAACCC
>‘990928a-050.scf” came from CONTIG 37 at offset 0;
“E:\SEQUENCE\export\EG_DB\990928a\990928a-050.scf’ (42>608)
GCACGAGGCTCGCTTCTCCCGGCCGCATCCCGCGGGACCGGAGCGAGCAGCGGGGACCATGTTCCGACGCAAGTTGACGGCCCTTGACTACCACAATCCTGCC
GGCTTCAACTGCAAAGATGAAACAGAATTTAGGAACTTCATTGTTTGGCTTGAAGACCAGAAAATCAGACACTATAAAATTGAAGACAGAGGTAATTTAAGAA
ACATCCACAGCAGTGACTGGCCCAAGTTCTTTGAAAAGTATCTCAGAGATGTTAACTGTCCTTTCAAGATTCAAGATCGACAGGAAGCAATCGACTGGCTTCT
TGGCTTAACTGTTAGACTTGAGTATGGAGATAATGCTGAAAAATACAACAAGGACTTGGTACCTGATAATACAAAAAATGCTGACAATGCAGCTAAAATGCAG
ACCATTGATCATTTGGATGTAAATATCCTGATTTAAAGGCTGTGTATGTCTTGGCTACCTTCTCAATCCACGTATGATGATACTGNAAGCTAAGCATTGCATT
TGTCCAGACGCCGACCAGAGCATGCTAGCAATAATGAGAGGTGCTGTGCTAA
>‘990928a-052.scf” came from CONTIG 38 at offset 0;
“E:\SEQUENCE\export\EG_DB\990928a\990928a-052.scf’ (34>527)
CACCGCCGGGGGGAGAGGAGGGGAGCCTGTCCGAGCCCCGGCCCCAAGTAACGCCGCCGCCCCGGAGCCGCCTTGGAGTCCTCCTCCCCACTAATTGCCTCTT
TGCATAGCACCGGCCCCTGCCCCCACGCTCACTGGTACCACTACAGCGGGACGGGCCATGGCGGGGCGGGGAGGCGCAGCGCGACCCAACGGACCAGCTGCTG
GGAACAAGATCTGTCAGTTTAATCTGGTCCTGCTGGGGGGAGTCCGCAGTGGGCAAATCCAGCCTCGTCCTCCGCTTCGTCAAGGGTCAGTTCCACGAATACC
AAGAATAGCACATTGGAGCGGCCTTGCTCACACAGACCGTCTGCTTGGAGACACACAGTCAAATTGAGATCTGGGACACAATGGACAGACGGATCACACCTGC
CCCCTGTATATCGGGGGCCCAGCTGCTCGGGGGCTACACATCACACACAAACCTTGACGGCAGACTGAGAAGANGTAAGAGN
>‘990928a-055.scf” came from CONTIG 39 at offset 0;
“E:\SEQUENCE\export\EG_DB\990928a\990928a-055.scf’ (31>112)
TTTTTATATCACAATCAATAGATTTACCAACAACCGGGAGCTGCAACTGCCTCTTTGAATGGGGGTGGGGTGGGGGAGNAGG
>‘990928a-056.scf” came from CONTIG 40 at offset 0;
“E:\SEQUENCE\export\EG_DB\990928a\990928a-056.scf’ (26>376)
CTGCTGGCCATACCTCTTAATAATACTTGATTTTTATACATTCACATGTTTTGCTCAAGTTTGTAGAAGACACTAGGAGAATTCCATTCCATTTCCTGGATGG
TTGCTGCTCTGGCTTTTTAAAACTTGGAACTAACGTTTATTTAAAGCAAAGAAAAATCTTTAAGAGGCTAAATTGATGCTGCTATTATTGCTGTGATTGTATA
AAGATTAGGATTCATGCCAGTTTTTTTAAATCTAAAAAATCATGTGATTGCATTTAAGGGTTTATATTTAAAAAAAGAAATAAAGTTTTAAGAGCAACACATT
TACTTATTATATGTGGAAATACTTGTTANGTGT
>‘990928a-056.scf” came from CONTIG 40 at offset 0;
“E:\SEQUENCE\export\EG_DB\990928a\990928a-056.scf’ (26>376)
CTGCTTGCGGACCTGCTGGAGCTCCTGGCACTCCTGGACCTCAAGGTATTGCTGGACAGCGTGGGGTGGTCGGCCTGCCTGGGCAGAGAGGAGAAAGAGGCTT
CCCTGGTCTTCCTGGGCCCCTCTGGGGAACCCGGCAAACAAGGTCCTTCTGGAGCAAGGGGTGAACGCGGCCCCCCTGGTCCCATGGGCCCCCCTGGATTGGC
TGGACCCCCTGGCGAGTCTGGACGTGAGGGAGCTCCTGGGGCTGAAGGATCCCACTGACGATATGGTTCTCCTGGCGCCAAGGGTGACCGNGGNGAGACCGGC
CCTGCTGGACCTCCTGGTGCTCCCTGCGCTCCCCGGGCCCCCGCCCCTGTCGGACCTGCCGGCAGACGGGNGATCGNGGNGAGACCCGTCTGCTGGTCTGCTG
GTCCATTGCCCGGTGTGGCCGNGGCCCCCNTGACCCAGCCCCGAGNGACAGGGGAGAAGGGACAGGCGAAAGATAGGGCACGGGGTTTTGTTCCAGCCCCCCG
CCTCGCTTCTGGAGATGACTCGAGCTTGTCTTGTGCCCCGCCCGCTTGTGTTTCGAATTGA
>‘990928a-058.scf” came from CONTIG 42 at offset 0;
“E:\SEQUENCE\export\EG_DB\990928a\990928a-058.scf’ (26>628)
CTGCTTTAATTCGGCACGAGGCAGTGACATTGTGCCCACATGGGACGGTATTCGGATGGGGGAGAGACTCCGCACCATGTCCTGCAGCGACAAGATCCTGCGC
TGGAACGTGCTGGGGCCTGCAGGGGGCACTGflGACCCACTTCCTGGAGCCTGTGTATCTCAAATCCGTCACTCTGGGTTACCTATTCAGCCAGGGGCACCTG
ACCCGCGCCATTTGCTGTCGTGTGACAAGAGATGGAAGAGCGTTTGAGGATGGACTTCGACATCCCTTATTGTCAACCACCCCAAGGTTGGCCGAGTAAGAGT
ATACAAATCCAAAAGGCAAGATGGGAGACCAAGAGAAAAGGTCAACTGGTGTTTGGGTGATGCTACGACTCGGATCTGGATGGACAGAGCACCGGGAGGACCA
CGGACGAATGTCCGGAGAACAAAGACATTTTCTTATTAAGAGCTTGTCTTCGATCCGAAAAATATTAATCTCTAGGGAGCAAAAAGTCCGGATACAAAACAAA
CTATTAAAATTTGAGAAGGCTAGGAAGGTACAGCCAGAGAAAATTTACTGCGGGTGGCGGGTGGGGGGGTTCAGGGGGAAAGGGG
>‘990928a-059.scf” came from CONTIG 43 at offset 0;
“E:\SEQUENCE\export\EG_DB\990928a\990928a-059.scf’ (34>633)
TTCAGAATGAAGCTGAACATCTCTTTCCCGGCCACTGGCTGCCAGAAGCTCATTGAAGGGGACGATGAACGAAAACTTCGTACCTTCTACGAGAAGCGTATGG
CCACAGAAGGTGCTGCTGACGCTCTGGGGGAAGAATGGAAGGGTTATGTGGTCCGAATCAGAGGCGGGAACGATAAGCAGGGTTTCCCCATGAAGCAGGGTGT
CTTGACCCATGGCAGAGTTCGCCTGCTACTGAGTAAGGGGCATTCCTGTTACAGACCAAGGAGGACTGAAGAGAGAAAGCGCAAATCTGTACGGGGTTGCATT
GTGGATGCCAATCTGAGTGTTCTCATTTGGTCATCAGGAAAAAAGGGATAAGGATATTCCTGGACTCACTGATACTACAGGCCCTGTGCCTGGGTCCCAAAGA
CCAGCAAATCCGCAACTTTCATCTCTTATGAANAGATGCCGCATATGTGGGGAAGCCCTAACAAACGTAGAAACTAGACTAAGCACAGATCACGCCTGGACTC
CGANTTGCACCAAGCGCGGTTGTTGAGATAGGATATAAAAAGAGAGTGGAATGTACTTGTAGAAGAGAGCAGAAAGAGAAGAGCA
>‘990928a-062.scf” came from CONTIG 44 at offset 0;
“E:\SEQUENCE\export\EG_DB\990928a\990928a-062.scf’ (26>467)
CTGCTGGTTTTTTTTTTTTTCAGTTTCTAAATCATTACTTTTTATTTTGAAAGATTTGTCAAACTCTTCACACCGGGGCAAGAGTTTGCATGATTAATAAGAA
GCAGTTTTTTCATGAAATGCTTGGAGGGGAACGAGTTCTCAGCCTGTGAGATCCGACCATCCCATTGACTTTGAAATTTCTTTTGATTAATATAAAGAAAAAG
TGGGGAGGGGAGAAGAGGAGGAACATGCTAGCGACTGAAAGATCTCTGGTGACAGCCATCCAAATGTGAAAAAAGAAAACAGAAAACCCAAAAGAAAACCAAT
TTGCCACCTGCCCTTTTCTGTTTTACCACGCTCTGCTCCTCGCTCTTGTTTTTGTCTTTCTGGTTGAAACAGCCTCCCTGCCCTGCATCTCCTAAAGAAGTTT
GGAGGGGAGCACCCCTGCCGACCGGGGACC
>‘990928a-063.scf” came from CONTIG 45 at offset 0;
“E:\SEQUENCE\export\EG_DB\990928a\990928a-063.scf’ (33>412)
TTTTTTTTTGGTAAGGTTGAATGCACTTTGGTTTTTGGTCATGTTCAGTTGGTCAAAGATAAAAACTAAGTTTGAGAGATGAATGCAAAGGAAAAAAATATTT
TCCAAAGTCCATGTGAAATTGTCTCCCATTTTTTGGCTTTTGGGGGGTTTTCAGTTTGGGTTGTTTGTCTGTTTCCAGAGTCAGGGGCAAGTGGGHGGGTGGG
AGGGAGCCAGGTTGGGGTGGAAGGAGTTTACAGGAAGCAGGCAGGGCCAACGTCGAAGCCGAATTCCTGGTCTGNGGCGCCAACGTCCAAGGGGGCCACATCG
ATGATGGGCAAGCGGGAGGTCTTGTTGTTTTTGTATTCGATCACTGTCTTGCCCCACGCTCCGTGTGACT
>‘990928a-064.scf” came from CONTIG 46 at offset 0;
“E:\SEQUENCE\export\EG_DB\990928a\990928a-064.scf’ (27>552)
TGCAGGTTCGCTTAGGGGCAGACGGGCAAACAGAGCCAGCATGCCGGTCGCCCGGAGGTGGGTTTGTCGCAAAACCTATGTGACCCCGCGGAGACCCTTCGAG
AAGTCCCGCCTCGACCAAGAGCTGAAGCTGATCGGCGAGTATGGGCTCCGGAACAAACGTGAGGTCTGGAGGGTCAAATTCACCCTGGCCAAGATCCGAAAGG
CTGCCCGGGAGCTGCTGACGCTGGATGAGAAAGACCCGCGGCGTCTGTTCGAAGGTAATGCCCTGTTGCGGCGGGTCGTCCGTATCGGGGTGCTGGATGAGGG
CANGATGAAGCTGGATTACATCCTGNGCCTGAAATTGAGGATTTNTTGGAGAGACGCCTGCAGACCCANGTCTTCAAGCTGGGCCTGCNCAAGTCCATCCACC
AGCCCGTGTGCTCATCCGCCAGGCCACATCAGGTCCGAGCAAGGGGGGACATCCCGTCTTCTCGGCGCCTGACTCGCAAAACAATCGACTCTCCTGCCTCCCT
CGCGGGGCCGC
>‘990928a-068.scf” came from CONTIG 47 at offset 0;
“E:\SEQUENCE\export\EG_DB\990928a\990928a-068.scf’ (34>622)
GTTGGATTTATTATTAAATTTTATTATGGATGCACGTACAAAAAATGAAGAATAACGACACACAACACTATGGAGAATAATTTAAACAAATTTTCTGTGATAA
TGAAACCAAAGCCGCCACTTTATTTATTGTTTGTTGTGGATGATTTTATAAGGATGGAGGGGATGAGGATTCTGGAGCGGTAATCGAGCATGCTTGTTTTTTA
TAAAACAAAATCTAACGGAGGACCTGGAATTAGATTGCAAGTCAATATTTTTGTTCTAGAGTTATCATAGCGAAAGACCATATGACCAAACCATGGTATTTAT
TGACTTGACTAATATTGAAAAAATGGACCAGGACCAACCAAGAATAAGAAATATTGCTTAGAACAAATGAATTCAAAATTTATTTGGCTTTTTTTTACTTTTT
AATTTTGAAGTCGTATGTTTTTTTAAAAAAATTCAGAATGTACGTTTGCCTGTTTCCTTCTTTTCCTTTGCATTTCAAAATTTGTGCTTTCATAAAGAGACTT
GTTCGAAAGCTTCTTATTTTGTTGAGTTTGTTAAGTCTTATTTGGGAACAATGTTTTGAATGGTAATAAAA
>‘990928a-077.scf” came from CONTIG 48 at offset 0;
“E:\SEQUENCE\export\EG_DB\990928a\990928a-077.scf’ (39>465)
GCACGAGGCCGAAGACGTCCTCGATGTCCAGCTGGCATTCCTCCGACTTCTCTCCAGCCGGGCCTCTCAGAACATCACATATCACTGCAAGAATAGCATTGCA
TACATGGATCATGCCAGTGGGGATGTAAAGAAAGCCTTGAAGCTGATGGGGATCAAATGAAGGTGACAAGGCTGAAGGAATAGCAAATTCACATACACAGTTC
TGGAGGATGGTTGCACAAAACACACTGGGGAATGGGGCAACAGTCTTCCAGTATCAAACACGCAGGGCCGTCAGACTACCTATTGTAGATATTGCACCTTAGT
ATCGGGGGTCCTGATCAAGATTTGGTGCGGACATTGGGCCTGTTTGGTTTTTTTATACAAACTCTTCCAAGACCACAAAAACTCACCTCCTATGTCTTTTGTT
TTATTT7GTC
>‘990928a-069.scf” came from CONTIG 48 at offset 0;
“E:\SEQUENCE\export\EG_DB\990928a\990928a-069.scf’ (34>497)
GGTGAATTCAAGGTTGAAGGAAATATTAAATTCACATACACAGTTGTGGAGGATGGTTGAACAAAACACACTGGGGAATGGGGGAAAACAGTCTTCCAGTATC
AAACACGCAAGGACGACATACTACCTATTGTTGATATTGCACCCTATGATATATGGGGGCCTGATCAAGAATTTGGTGGGGACATTGGGCCTGTTTGCTTTTT
ATATACCAAACTCTATCCTAAGTCCCAGAAAAAGCTTCACACTCCATATGTTGCTTTTGTTCTAATCTTGTCAGCCAGGACAAGCGACCAAGTTCCAGTTATT
TATTTCGAAAATTCTTGGGAAAAAGNGCAATTTGACCAAAAAGATATTTGTTTTGCTATTACCCCAATACAGTCAGAGCTTATTGTTTATTTTTTTCCATTTC
AATTCAAAAGCCTCATGCTGCTATAATAAAAAACTCACACTCTCCATACACT
>‘990928a-070.scf” came from CONTIG 49 at offset 0;
“E:\SEQUENCE\export\EG_DB\990928a\990928a-070.scf’ (34>475)
CGCGAGTGGCATATTGGTTGCTACCTTGCAGGCCTGTGCACCCTGGAACCTGCCCTAGAACTTTGTGTGCTGGACTTGGGGAGGAGGAATCCTGGGGCTGGAT
ACGTTCCTTCTAAAAACTTCCCCCTTCAAGTGCAAGAGCGGCATCTGGATCATTGAGGAGTGTGAGGGCAGGTTTCTGGCTCACAGGGACCTGCCGCACACAT
ACCTGAGAATCAATCGCTTGCTCCTGTGCCATCTAGAGAGGTGTGGACACCTGATGACTGTGGAGACACCACCAGGGGGAACGTAGCATTGTTACTGGCTGAC
TCCAGCAAGTCTGCGCATGGAGCTGCTGATGCCCCCTTTCACTATCCTCCGGTCTTCACCTTCCCACGTATGAGATGACTACCCTCTATATATAAACGNTATG
ANGCCACCTGCCTACTCCCCTCCCACACAC
>‘990928a-072.scf” came from CONTIG 50 at offset 0;
“E:\SEQUENCE\export\EG_DB\990928a\990928a-072.scf’ (34>259)
GGTGAGCGTGTATGGGGGTGCCCAGTCCCAGGAGAGAGGGGACAGAGAAGGACAGGCCTGTAGTCATTAGGATGGGCCTTCGTGCTGAGTAGCAATGTGTATA
CCATTTGGGCTATCAGAGGTACCCCTGGGCAGGAGCCTCCACACACCCCCTTCCCTCTTCTCTCTCCATGACTCTTCTTCACATCCTATCTTTCTTCTAAGAG
GGGGAGGGTAGGGGAAAATTT
>‘990928a-075.scf” came from CONTIG 51 at offset 0;
“E:\SEQUENCE\export\EG_DB\990928a\990928a-075.scf’ (34>577)
CACGGAAGACTGGGAGTCTGTTCTGGGGCCAGAAGATCCGGGCAGGTGTCCAGCCAACAGCTGACCGCAGACTGCGTCTTGTCTCCGTTTCCGCAGGCACCAT
GAGCCAGAACACCGAGGTGGGCATGAAGGAAGTGGAACTGAACGAGCTGGAACCCGAGAAGCAGCCGATGAACGCGGCGTGTGGGGCGGGGATGGCCGTGGTG
GTGGGGGGCGGCACCGAGAAGAATGGTCTGGTTAAGATCAAGGTGGGCGACGACGAGACGGACGCAGCGGCCGAGGCCAAGCTCACGGGCCTGTCTAAAGAGG
AACTGTGAAGGTGGGGGGCAGCCCCGCCTGGGTACGCACCCGCTGGGCGCTGCTGCTGGTCTTCTGGTTGGGTGGTGGGCTGCTGGCGCGCCCGGGTCATCAT
TTGCAGCGCCACCCTGCGCGAGCTGCTACAGAATGAGGGCAAGGGGCCTCTACGCATTGAACCTCGGCCTCCTGGCCAGAGCAGCAACTACGACCTAAGAAGG
AGGATACTAACCCTGAGGAGGTTTGGTGG
>‘990928a-079.scf” came from CONTIG 52 at offset 0;
“E:\SEQUENCE\export\EG_DB\990928a\990928a-079.scf’ (34>357)
TCTTGAAGATCTTTTTTGTGCAGTTCTTCTGTGTATTCTTGCCACCTCTTCTTAATATCTTCTGCTTCTATTAGGTCCATACCATTTCTGTCCTTTCGAGCCC
ATCTTTGCATGAAATGTTCCCTTGGTATCTCTAATTTTCTTGAAAAGATCTCTAGTCTTTCCCATTCTGTTGTTTTCCTATATTTCTTTGTATTGATCTCTGA
GGAAGGCTTTCTTATCTCTTCTTGCTATTCTTTGGAACTCTGCATTCAGATGCTTATATCTTTTCTTTTCTCCTTTGCTTTTTGCTTCTCTTCTTTTCACAGC
TATTTGTTAGGCC
>‘990928a-083.scf” came from CONTIG 53 at offset 0;
“E:\SEQUENCE\export\EG_DB\990928a\990928a-083.scf’ (41>427)
GCACGAGGAAACCGTGGTGAACCGGGTCCTGCCGGTGCTGTTGGTCCTGCTGGTGCTGTTGGCCCAAGAGGTCCCAGTGGCCCACAAGGTATTCGAGGTGACA
AGGGAGAGCCTGGTGATAAGGGTCCCAGAGGTCTTCCTGGCTTAAAGGGACACAATGGGTTGCAAGGTCTCCCGGGTCTTGCTGGTCATCATGGCGATCAAGG
TGCTCCCGGTGCTGTGGGGTCCCGCTGGTCCCAGGGGCCCTGCTGGTCCTTCTGGCCCCGCTGGCAAAGACGGCGCATGGACACCTNNNNGCAGTGGACTGCT
GGCATTCGTGCTCTCANGGTAGCCAAGTCCTGCTGGCCTCCTGTCCCCCTGCCCTTCTGGACTCTGCCAGGNGGGGGG
>‘990928a-084.scf” came from CONTIG 54 at offset 0;
“E:\SEQUENCE\export\EG_DB\990928a\990928a-084.scf’ (36>436)
GCCGGTCTGGAGGAACTGGGGTCGACCTGAGTTCTGGGGGTGTGGTAGGCCAGGGCGGGGTGTGGCCTCCCTCCAGGCGCCATCATGATCATATACCGGGGAC
CTCATTAGGGCATGACGAGATGGTTTCCGACATCTACAAGATCCGGGAGGTAGGGGGCGGGGTGGGGGTGGAGGGGGAGGGGTTTGATGGTCAGTAGGACAGA
GGGTAACATCGATGACTGGCTCATTGGTGGAAATGCCTCCGCTGAAGGCCCCGAGGGCGAAGGTACGGAAAGCACAGAAATCACTGGTGTCGATATTGTGATG
AACCATCACTTGCAGGAAACCAGCTTTACAAAAGAAGACTACACTAAGTACATCAAAGATTACATGAAGCCTATCAAAGGGAAACTTGAAGAC
>‘990928a-085.scf” came from CONTIG 55 at offset 0;
“E:\SEQUENCE\export\EG_DB\990928a\990928a-085.scf’ (41>633)
GCACGAGGGCTATCCTAATCGCAATTGGAAGGATGCAGGGCTGAAGAACGGTGTACCAGCTGTGGGCCTGAAGCTTAATGACCTCATCCAACGGTTGCAGCTG
TGCTACCAGCTCACCACAGTTGGCAAGTTTGAGGAGGCTGTGGAAAAATTCCGGTCCATTTTACTCAGCGTGCCACTTCTTGTTGTAGACAATAAAGAAGAGA
TTGCAGAGGCTCAGCAGCTTATCACCATTTGCCGTGAGTACATTGTGGGTTTGTCCATGGAGACAGAAAGGAAGAAACTGCCCAAGGAAACTCTAGACAGCAG
AGCGCATCTGTGAGATGGCAGCCTATTTCACCCACTCAAACCTGCAGCCAGTGCCATGATCCTGTGTTGCGTACAGCCCTCACCTCTTCTTCAGCTCAAGACT
CAGNATGCTGCCGCCUGCTCGCGCCTGTTGGACTCGGCCCCAGCGGAGGGATCACAACCGCAAACCTGTTGCTGGAGAAAACCAANAGCCATAATCATATACA
TGACACCTTGAATTGGCTGTCTTCGCCCTTACGGAAGCAGGAGAAGACGTAGGGGCTGTTTCTGTTAGTAGATGAGA
>‘990928a-087.scf” came from CONTIG 56 at offset 0;
“E:\SEQUENCE\export\EG_DB\990928a\990928a-087.scf’ (35>492)
TTTTTTTTGAAAGAAACATCGCTTTTTTTATTTTTTACACAGTTCAACTGGGACACAAGATATTCTATAAAAATTCCCAATAAGAGCTAAAGAAAACCCTTTG
TGTATTTGGTACATATTACTCTAAAAAGTTTCTGAACTCTGTTTAATAGCAGGTCTCCTCTTGATAGTTNTTTGGTACTGCCAGTTTTTGTTGTCAGGTCTGT
TGGTTTCCACTACTTCATTCACTTTATCTAGCAGTGAAGCCTACCAGCACCAATAACCTGGAGAGTTCCTGATCAATGAACTCCACACCAACACCAAAAGCTT
ATGCCATATAGACCAGGAGTAATGAACTGCACGATTCCAGCTGGTGGCTGTACGCATGTATTGGCATTTCTCTACGTTATATCGATAATGAGAGCAAAAGTCA
TCCTTTTTCTTTGCTTTCCCCATATCTACACT
>‘990928a-089.scf” came from CONTIG 57 at offset 0;
“E:\SEQUENCE\export\EG_DB\990928a\990928a-089.scf’ (42>552)
GCACGAGGGGGGAGAAAAGGGGTCAGGTTCTTCTCGTGTCAAGCAAAATTCTGCCTCAACTTAACTATTGCAGCAGGGTACCCAGGCCTTTCGGGAGTCCCTG
GACTGTCTGCCTAACCACATGCAGTCAGCCGCTTAACGCTACACCAGTCTCATCCCCTCTGTCCCCCATTCACAGGCCGGGGGAGTGGGGGAGACAGTTGGCC
ACCTTCCTCTGTATATTAATTGGCATGATATTGGTATATAGGATTTTAGCAATTCATGATAAATATGTCAGGCTAGGCTTTACTATTTTAATGCTTATGGACA
TTGTATATTTGTATTTTAAGACCAAGTAGACCAAGTCACAAAGGTCTCTCTCTAGCGTACCATACACCCGNGGCCGGNAGAAGGCTGGAAGGCTCCACCAGTT
CTGAAGGCAGCTGCTTTCCTGTCCTCTGTGCTGGACGGCCCTGTCCACTATAACTAAACTGGCTCCGAGGGTACGCATAGNGGCTTACGTCTCA
>‘990928a-091.scf” came from CONTIG 58 at offset 0;
“E:\SEQUENCE\export\EG_DB\990928a\990928a-091.scf’ (43>555)
GGACGAGGCGCCTGCGGAGCGGCGTGCCCGGCGTGCGGGTGCTGCAGGGACTCGGTGGACTTCTCGTTGGCCGACGCCATCAACACCGAGTTCAAGAACACCC
GCACCAACGAGAAGGTGGAGCTGCAGGAGCTCAATGACCGCTTCGCCAACTACATCGACAAGGTGCGCTTCCTGGAGCAGCANAACAAGATCCTGCTGGCTGA
GCTTGAGCAGCTCAAGGGCCAGGGCAAGGCGCGGCCTGGGGGACCTCTACGAGGAGGGAAGAGCGAGAGCTGGGCCGGCAGGGGACCAACTCACCACGACAAA
GCCCGGTCGAGGGGGAGGCGACAACCTGCCGAAGACATATGAGGGTCGGGAGAAGTAGGAGGAGAGAGCTTGAAGCGAGACATCTGCATCTTTTAACAGATGT
GACATGGCTTTTGGCCGTTTGCCTGGAGGAAGGGGATCTTGAAAAAATGACTTTTATAATGATAATAGAATCAGAACTAGCCAATCATAACAA
>‘990928a-095.scf” came from CONTIG 59 at offset 0;
“E:\SEQUENCE\export\EG_DB\990928a\990928a-095.scf’ (44>509)
GCACGAGGGAACTAAGGAACATATTGGACTTAACAAATGTGCTCTTAGAACTTATGTGTGTGTAGGGGGCTTACTGTGCTCTTTCTAAATAAAAGAAATATTG
GGGCTACATGAGTAGCACTTACCCATTTCTCATTCTCCAGTCTCTCGATATTGTCAGGGATAGACTAAACAGAAGACTTGTTTGTTTTTATACTAGCCAAAAA
TGTTATAACCTCTAAATATTCCAAAAATTATACATTAAGAATAACAGAATCAGAACATAAAATTCTAAAATAACTCTCAAACTGTGTAGATTAACATCTTGAT
TTATATTAAATCTTCAACAGAATAGAGAAAGTAACTCTGATTTACAAGGTCCCAGTATTTTTCTCTTTATATGACAGTTGTACATAGTGAAGTGAGGATATAA
AACCAACGAACTGTGATTTAGGATAGACTGGGGGTATTTGTCAGCAGGTTGGG
>‘991015a-001.scf” came from CONTIG 1 at offset 0;
“E:\SEQUENCE\export\EG_DB\991015a\991015a-001.scf’ (42>686)
TTTTTTTCGGCACGAGGATGAGCTGGGTAGACCTTATCATCCCAGATACCCTATAAGCTCCTCCTTGCTTTATGGATAGCATTGCTGAAGATCACCGGCTGGA
GAGCCCCACTACTGCTCTGCTAGAGGACACAGATGGTTCTCAAGACAGCCCTATTTTTATGTATGCTCCTGAGTTCAGGTTCATGCCACCACCTACTTATTCT
GAGGGTGATCCCTCCGTCCTGAACACAATGTGCAGTGAGCATGTGGAAGAAAAGAAGCAGTTGTACCTACCTGTTTCTCTTCATCTCTCTTCCTGGACTCTTT
AATTTTTTAGAGACTCAACAGTCTCCACAGGGGGGGACAGGCCCACCCCACCCTCTGTCTCTCCAGGGGGGAGGGGGACAACAAGCAACCCCTGGGCCCTAAG
GGGCGCACCCCCCTCACCACCCAATGATGGGATACNAAATTTTTGTAATGGGTTAAAACACCTACAAAAAAACTAGCCCTCCCATTCTTGATTCTTTTAAAGA
GGATTTCAGAATTTGATAAGGGAGAAAGGACCGGAGCGGCCCCGCGAAGGGGGATATAAAAAAATGTTAAAAAAACTACCCGCCCGGGGGAACATAAAACAAA
AAGAAAAAAAGAAGGCAATGGAAAAG
>‘991015a-003.scf” came from CONTIG 2 at offset 0;
“E:\SEQUENCE\export\EG_DB\991015a\991015a-003.scf’ (45>709)
GTTTGCCGCCAGGACACAGGTGTCGTGAAAACCACCGCTAAACCTAAACCAAAATGGGAAAGGAGAAGACCCACATCAACATCGTTGTCATTGGGCACGTAGA
TTCAGGGAAGTCTACCACGACTGGCCATCTGATCTACAAATGTGGCGGGGATCGACAAGAGAACAATTGAAAAGATCGAGAAGGAGGCTGCCGAGATGGGAAA
GGGCTCCTTCAAATATGCCTGGGTCTTGGACAAACTTAAAGCTGAACGTGAGCGTGGTATCACCATTGATATCTCCCTGTGGAAATTTGAGACCAGCAAGTAC
TATGTTTACCATCATTGATGCCCCAGGACACAGAGACTTCATCAAAAAATGATTACAGGCACATCCCAGCTGACTGTGCTGCCTGATCGTTTGCTGGTGTGGT
GAATTTGAGGCGGGATCTTCAAAACGGGCAGACCCGCGAGCTGCCCTTTTGCTTAACCTGGGGTGAAAAAATAATTGCGTAGAAAAGGATTCATGACCCCCTA
TACCAGAGAAAAGAAAAATGTTAGAAGCAGACCTATTATAAAATGGTACACCCGCACAAACTTTGCCCATTTGCTGATGGGCAAAGCTAACAAGCTATATCCG
GTAGGGGGGAAGACGAGGAGCAGCCGGACCCCGTTACTCGGTGT
>‘991015a-004.scf” came from CONTIG 3 at offset 0;
“E:\SEQUENCE\export\EG_DB\991015a\991015a-004.scf’ (51>626)
GCACGAGGGAGCTGCCGGGCCGCCCAGAACACCGCTGGTTCCCCTGGCAACCCAGGAGAGCCAAACCCTACTCGGGTGGTTGCAGCGAGAGCGGGCGCGATGG
TGTCTAACCCAGTGCATGGCCTGCCCTTTCTCCCAGGCACGTCCTTTAAGGACTTAACGAAAACAGCATTTCATAGAAGCCAGACTCTGGGCTACAGGAATGG
CTACGCAGTTGTTCGACGTCCAACAGTTGGGATTGGTGGGGACAGGCTCAAGGTCAACCAGCTGTCCCAGGCTGATCTGGATGAGCTGGCCAGCAAGATCCCA
ATCCTCACGTATGGACAGGCCAGGCAGGCCCCGCCTGCAGCGTTTGTTCCTGCACATGTGGCTTTCGACAAAAAGGGGCTTAAAATTGATGCCTATTTCAGAA
GATGTTTCTATGTCATTTGAGAAAATTACAGGATTCGCCAGTGACATTTATATTATTAAAAATGAAGCTGCCGCATAGACCTGGCGGGAAAAATTGGGTACCT
CAGGAAGTTATAAACCCAGGGCTTCCAATGACGGGGGACATACCTGAAGACTAAACGAAA
>‘991015a-010.scf” came from CONTIG 4 at offset 0;
“E:\SEQUENCE\export\EG_DB\991015a\991015a-010.scf’ (44>590)
TTTTTTTGTATTAAAAAGAAAAGCATAATTACCACAAATTACAAACGACTAAAGCAGGACTAGAATAATGAATGAATCACTTCAGCCTGAAAAGCAGATACTC
TCAATAATATTAATGTTATAATACAAGCTCATTCAAGTATTTTACATTTTTTTTTCCTTGTAAATGTGATATCCTAGCACATGGTAAAGATAATGTACTATGA
GCATAAGGTGGAACCTTCTCCGCAAAGCCAGATGACAAGTTTTCCTCTCCAGTCAGATGGGCGGATGTTCTGATCTTCTTGCCTCTACCATCATGTGGCTGAA
GAGAGGGGGGGGCTGGGGCAAACCTGGGACCCTACACCACAAATACTGGGGGGCCCATCAGAAGGGGAACCAAACACCTATTAGGTTTTGGAAAAACAGCCCC
CCCCTACTAGTGGTTTTTTGCCCTCCTCCCTGCAGGGCTTTGACAGTTTTTTTGAAATAACACAGAAAGGGAAGAGAATAAATAACAAACAAATACACTTGAA
TGGGGCCTCCTGGCTTGACCAGGGGCAGCGGG
>‘991015a-012.scf” came from CONTIG 5 at offset 0;
“E:\SEQUENCE\export\EG_DB\991015a\991015a-012.scf’ (41>316)
TGCTGGGCCGGGAGCGGCAGCGGGTGCAGGGGGAGCTGCAGTCCCTGAGCCAGCGGCTACAGCAGGAGTTTGTGCCCATGGCCGGAGGCGCAGGACCAGCTAC
AGCAGTTGGGGAGGAGTGTGGGGCTGCGTGACATGAGAACTTGTCCCTGGAAAATCTGGCCCACCGAGAAGGCTTGTGGAAGCCGCCTGGGCTTTTTTGTTCA
GATGTGTTCTGGGGGTTGGGGGTTTGGCTGGTCTCTGACCTGGGTGGGTCAGGGGAGTGTTGGGGACGGT
>‘991015a-013.scf” came from CONTIG 6 at offset 0;
“E:\SEQUENCE\export\EG_DB\991015a\991015a-013.scf’ (49>665)
GCACGAGGGGCCCTATGGCCAGTGCAAGCTGCTGCGCACGCACAACTACCTGTACGCGGCCTGCGAGTGCAAGGCCGGATGGCGGGGCTGGGGCTGCACGGAC
AGTGCGGAGGCGCTCACCTACGGGTTCCAGCTGCTGTCCACACTACTGCTCTGCCTGAGCAACCTCATGTTTTTGCCACCCGTGGTCCTGGCCATTCGGAGCC
GATATGTGCTGGAAGCGGCAGTCTATACCTTCACCATGTTCTTCTCCACGTTCTACCATGCCTGTGACCAGCCTGGCATTGTGGTTTTCTGCATCATGGACTA
TGATGTGCTGCAGTTCTGTGACTTCCTGGGCTCCTTATGTCCGGTGGGTGACTGTTATTGCCATGGCTCGTTTACAGCCTGTGGCAAGCAAGTGCTATATTGC
TGGGGCGATGCTGCTGGCATGGTTCTGAGCTTGACGGATGGACTCTGGACCTGTTGGACCGAGCTTTTTGTCTGGGATTTGGCACAGCCGGAAGAACCAAGGC
GCCGCGGGCTGTTTCCCCCCCAAGGGCGGTGGTTTTTGCTGGCCGGCAGCCTCGAAGAGGCATCTGCGTTTGTGTGAGACGAAAACATTAATTAACATTGA
>‘991015a-015.scf” came from CONTIG 7 at offset 0;
“E:\SEQUENCE\export\EG_DB\991015a\991015a-015.scf’ (44>735)
TTCGGCACGAGGGAAGAATTTAGCTGCTGCGTATGACAACTTTGTTGAACTTGTGGGTAATTTGAAGGAGGGCACAAAGTTTTACAATGAGCTGACTGAAATA
CTTGTCAGGTTCCAGAACAAATGCAGTGATATAGTGTTTGCACGGAAGACAGAAAGGGATGAACTCTTAAAGGACTTGCAACAAAGCATTGCCAGAGAACCTA
GTGCTCCTTCAATCCCAACACCCACATACCAGTCCTCTCCAGCTGGGGGGCACGCACCAACGCCACCAACTCCAGCACCAAGAACCATGCCGCCTACTAAGCC
CCAGCCCCCAGGCCGACCTCCACCTCCTGTGCTGCCAGCAAATCGAACTCCTGCTTCAGCTCCGGCTGCAGCTCTAGCTCCAGCCCCGGCTCCGGCAGGCTCT
GGGACCACTGTACCAGCTCCATCACAGACNCCCGGGTCAGGTCCCCTGCCTCAGGCGCAGGACCCCCGCACCCACCTTCCAGGGATCCCGGGATTTGCAATGC
CATGCCCTGGGCTACATCCCTAGCGGATGGCAGATATATGCGGATCCACAGGATACAAAACCTGGCAGCTCCTACCCGAGCCCACACTCTACCCTGCTCACCC
CCCACACCTTCCCAAAAAAAGCAGTAAAAACAATGTATTAAAGAAAAAACAACCGCAAAGACACCTTACTTG
>‘991015a-022.scf” came from CONTIG 8 at offset 0;
“E:\SEQUENCE\export\EG_DB\991015a\991015a-022.scf’ (48>641)
GCACGAGGATCGTCTTTAAACCCTGCGTGGCAATCCCTGACGCACCGCCGTGATGCCCAGGGAAGACAGGGCGACCTGGAAGTCCAACTACTTCCTTAAGATC
ATCCAACTTCTGGATGATTATCCAAAATGCTTCATTGTGGGAGCAGACAACGTGGGCTCCAAGCAGATGCAGCAGATCCGCATGTCCCTCCGCGGGAAGGCTG
TGGTGCTGATGGGCAAGAACACGATGATGCGCAAGGCCATCCGAGGGCATCTGGAAAACAACCCGGCTCTGGAGAAACTGTTGCCTCACATCCGGGGGAATGG
GGGCTTCGTGTTCACCAAGGAGGACCTCATGAGATCAGGACATGCTGCTGGCCAACAAGGGGCAGCTGCGGCCCGGCTGTGGCCTAGGCCCGGGGAAGGCACT
GTGCCAGACAGACACTGGGCTGGGGCCGAGAGAACCTCTCTTCAGCTTTTAGCTAACACAAAAATTCAGGGCACAATGAATCTGATGAGGGCACTGATAAGAA
GGAGAAAGAAGCGCAGCGAGCAGCTGTGACTGCGACATTCCCTCTCTTGGCTGGCTTCACAGGTTGAAGGAGAAAAAAA
>‘991015a-025.scf” came from CONTIG 9 at offset 0;
“E:\SEQUENCE\export\EG_DB\991015a\991015a-025.scf’ (42>669)
TTTTTTTTTTTTTTCAACAAACAACAAATATTTATTGAGCGCCTATTATGTGCCAGGCACTGTTCTAGAATCCCCCCCAAAAAAGAAAAACAAGATAGATGCA
GAAAATGCAAATTCTGAGGGAGAGGAAAGGGGTCGTTGAGGAAGAAGGCTGAGGGGATTAAAAAGCCTAAGGTGATAGGGACTTGGCCTTAGGCCTCCTCTTC
GGCCTCCTCACCGAAATCCTCTTCCTCTTCTGCGGTGGCATCCTGGTACTGCTGGTACTCGGAGACGAGGTCGTTCATGTTGCTCTCAGCCTCGGTGAACTCC
ATCTCGTCCATGCCCTCACCTGTGTACCCAGTGGAGGAAGGCCTTCCGGGGAACATGGCCGTGAACTGCTCCGAGATGCGCTTGAACAGCTCCTGGATGGTGT
GCTGTTGCCATGAAGGTGACTGCCATTTTATACCACGNGGGGGATGTCGCAACAGTTGTTTGACGTGTGGGGTCCATTCACGAAAAACTGTGTTTTATCTGAC
GTGAGCTTGCTCTJTACTCCTCTTGGACTCGCCCAGAACCACAGCCCGGGAGACCGCCTGCGGGGCAAGGCGCTCTGTCTTGGTCGAAATGTGGGAGCCAGAC
GGGGGCCGGA
>‘991015a-028.scf” came from CONTIG 10 at offset 0;
“E:\SEQUENCE\export\EG_DB\991015a\991015a-028.scf’ (42>718)
CCGCTCCGGAGCTGCTGCCGCTCCTGCTCTCAGCGCTGCAGTGGAAGGCAGGGCCGCGCCGCGCCGCTCCTTTTTGAAATATATAAATTGGAGGCCGGGCCGG
CCTCGGCGCGCCCCTCTGACAGCGCGGGCCGCGCCCCGTCCCGCCGGCGCGGCCGCTGCCAGCCCCGGGACCTTTTCATCTCTTTCTTTTTGGGCGGAGGAGG
CGAGTTTAGATTCGCCACTCCGGACCCGAAATTGACACACTGAACTCCGTTTTCTTCTGCTAAATTATTTCTGCTTAATAGCCACTCGTTTTTTTTTTCTGTC
CCCCCTCCCCTCCATCTCGTTGCTTCAAGAAAACTTTGGCGACTCCCTAAGTGCAGGTTTCCCTGGCCGGCGTGTTTTTATTTTTCATTTGGGGAACGAGTTG
GTTTTTTTTTGAAAGAATTTAGATAGATACTTTTTTTTTTGGCCTTGATTTGATTGTTGCAAAGTTTGATTAAATAAATTTACTGTTTGTTTTGAAGTGTGGG
TTTTTTTTTTTTTTTTTTTTTTAAAATAAATTTTTTACTTTAAATGTTATGTGGTGGGCGTTTTTTGTTTGCCGGGGTTACTGTTCTTTCACCGTFGCCTTCT
GCAAGTGGGTGGGGGCATTGATTTATTCCCCCCAAAATCCCCCCGGGTCCGGGTTCTCACA
>‘991015a-029.scf” came from CONTIG 10 at offset 0;
“E:\SEQUENCE\export\EG_DB\991015a\991015a-029.scf’ (42>653)
CCGCTCCGGAGCTGCTGCCGCTCCTGCTCTCAGCGCTGCAGTGGAAGGCAGGGCCGCGCCGCGCCGCTCCTTTTTGAAATATATAAAATGGAGCCGGCCGGCC
TCGGCCGCCCCTCTGACAGCGCGGCCCGCGCCCCTCCCGCCGGCGCGCCCGCTGCCAGCCCCGGGACCTTTTCATCTCTTTCCITTTGGGCGGAGGAGCCGAG
TTCAGATTCGCCACTCCGCACCCGAAACTGACACACTGAACTTCGTTTCCTCCTGCTAAATTTATTTCTGCCTAATAGCCACTCGTCTTTTTTTTTCCGCCCC
CCCTTCCCTCCCTTTCGCTGCTCCCAGAAAACTTTTGCCGACTTCCTCAGTGCAGGTTCCCCCTGCCCGTGGTGGATTATATTTCACTTGGGAACGACGTGGG
CTTTTTTTTTGAAGGGATTCAAGCAAGATCTTTTTTTTTGGCATTGGCTCTCGATGGTTGCAAAGTTGGCATTAAAAAATATTCCTGTCTGGACTTGGGAGTG
TTGGGTTTTTTTTTTTTTTTTTTTTATTTAAAATAAAAATTTTTTTCCCTTTAAAAAGCCTTTTGGGGTGGGGGGTTTTGGTCTGTTTGGTCGGG
>‘991015a-030.scf” came from CONTIG 11 at offset 0;
“E:\SEQUENCE\export\EG_DB\991015a\991015a-030.scf’ (46>759)
GGCACGAGGGAGATGTTCGAGCCCGGCCGCATGCACCGCGTGGGGGCGCTCGCGGCCCATAACTACCTGCAGACCCCAGCGGCCGGCAGCTCCGCGAGGCCGC
GCAGCGCTTCCGCCGCTGGCAGGCCCAGCGCCCAGACTGGTTCGAACTGGAGAACCTCCGCTCAGCGGACGGCCGGGACCTCCCGTCCCTGGACGAGGAGGCC
TTCGAGGAGGAGCCGCTGCCCGAAGAGGGATCGTCCGGCAGGAGCCGCTGGTGCGGGAGCCCACCTCCCGGGACCACCTGCTGGTGGGAGCTGCTCCTCGCTG
GAGAAGGAGGGGGCGGGCTGGGGCGGGTGGAGCCCCAACCCCGGGCCCGGGGCAGCCGGCAGCCCCAACGCTCCATACATGGTGGTCCGGGGGGAAGGTCCCT
CGGGCTAGGAAGGGAGCCCGTCCACAGGAGTGGGAGCGGGCTGGCGGGTGGCCTAATGAGGGAAAGAGCCTGCCGCTTTGGGAGCCAGGACCCGGGGAAAAGT
CCTTTCTTTCGAGGGGGGTCCGACCCCACTGAACCAACTCAAAATGCACCCCCGGGGGACAAGGGGTGGGAGCGGAAGTTTTTTCAGACCCCCACCCCCCAAA
AAAAAAAAAGAGGGGGGGGGGGAGGGGGGGGTCCCGGCGGAAACGCACCCCTGGTTTTTATATAAAACCCCTGCCGGGCCTGGGCTCCCCCCCCCT
>‘991015a-040.scf” came from CONTIG 12 at offset 0;
“E:\SEQUENCE\export\EG_DB\991015a\991015a-040.scf’ (47>728)
GCACGAGGGTTAGAAAAAGAATACATGATYCTAGAGTTGCTGGTTTTAACCCTGCACTACAGTTAATCCTGACGAGGACAGACAAAACCTTAAACAAAAAACT
TGGCCAAAGCAAATAACTTCTTAAACAGTCAAGATCTTGGAGACTCTAGAGGATTTTTGTACTAGTAGCCCAAGGAAGATGGAAAAATGACTCCTTTTTAAAT
CTGATTCAGACTGTCACTGCTTTGGGAAACTGCTAAATTGTTGGATCTGCTTCCAGGGTCTATGCATCTCTTGTTCAGCATGAATTTAATTTAGTAGTCCAAG
CCAGACTGTTCCTCTGGAACCTGACTAATCCATATTTCCTATGTTTTGGAATTAGCTTGCTCCTCCTAAAAATCTCCACAGCCTAATATTGATGTTATTATGA
CAGGGAAGCCTGAGGAGAAGCCTATACTGTGTGGACTTTAATTATATTCTAGAATAACTTTTGGGGAAAGCTCNCTGTAGCTGATTCTAGATTATCCAGAAAA
CAATTAACTTTAATAAAACTATTTGTCTAATCCCCTCTATPATATCAGGATACTATTTAATATTAGATATTATTATTACAAACATCTATGGTTTTTGTTATCT
AGGGGTTGATTTTACGTCCGCAAAAACCATAAAGCAACCATCATAAAAAGCAAAGCCTTATCTG
>‘991015a-044.scf” came from CONTIG 13 at offset 0;
“E:\SEQUENCE\export\EG_DB\991015a\991015a-044.scf’ (47>695)
GCACGAGGGCCGAACGCAACGAGCTTCGCAGGAGGCGGTGGGAGCGACGGCGGGGGGCTGAGCTTTGGGGCGACCCCGCTTCCCTCCTCCCAATCGCTTCGCT
TCCCTTCCCCGCGGGCAGCATGAAAGCCTTCAGTCCAGGGAGGTGCGTTAGGAAAAACAGCCTTTCGGACCACGGGCTGGGCATCTCCCGGAGCGAAACCCCG
GGGGACGACCCGATGAGGCTGCTGTGCAACATGAACGACTGGTACTCCAAGCTCAAGGAGCGGGGGCCCAACATACCGCAAAAAAGAAGGAGAGCAAGAGGAA
ATGCTGGAGGACGAGATGGACTACATCTTGGACTTGAGATCGCGCTGGACTCGCAACCCCCATTCGTTAGCCGGGCCACCAACGACCCGACAGACCGGCGTCC
AGGACGCGCTACCAACTCAACACGACATAGATCTGGCCTGCAGAAAACCCCTCCCCCGCGAGCCGGGAAGCGGAGCAGGCGAGGGCCTAGGCTCATTAAAAAG
ATAAATTTTATACTTCTGTTTGTTTTAAAAAAAAAATGTGGGAAGAAGGGGGGAATGTAATATAAAAAAAGCCCAACGATTAATAAAGGGGGGGAATGGGAAA
AAAATTTTTTTAAGTTTTCTTTGGGGAGGGG
>‘991015a-046.scf” came from CONTIG 14 at offset 0;
“E:\SEQUENCE\export\EG_DB\991015a\991015a-046.scf’ (47>699)
GCCCGAGGGCCAGTTTCTCAGTGAACATGCAGGAAGTTCAAACGCCTTCTCTAGTGGAGAGCATACCAATTACTATTTTGATGTTTCTCATGAACACCTAGAA
GGTGCGCTAGACAGATTTGCACAGTTTTTCCTGTGCCGCTTGGTTGATGAGAGCTGCAAAGACAGAGAGGTGAATGCAGTTGATTCATAACATGAGATGAATG
TGATGAACGATGCCTGGAGACTCTTTCAGCTGGAAAAAGCTACAGGGAATCCCAAACATCCCTTCAGTATATTTGGAACAGGTAACAAATATACTCTAGAGAC
TAGACCGAACCAAGAAGCATTGGATGTAGACAAGAGCTCCTGAATTCCATTCTATTTATATTCATCGAACTTATGGTATTTGTGTTTTAGCCGAGATCTTTGA
TGATTTGCTATCTGGTGGGAAGGTATTTGTGAAGAGAGACAAAATGCCCCGTGCCGATTTCCGACATCTTTTCCAAGAAATCTTAACACTTTCAAAATACCCA
TTAGTTTTAGATCTTTGGACATCCCTCCTGCTCCCAAACACAACAACCCGCCTTTCCGCTCCTTGGGAGAGGCCGAGCGTTTAACTTATAAAGCGGGAATTTT
GGGGGAAGGAGGACGGGGTGTTTTTTTTTTGGGGT
>‘991015a-047.scf” came from CONTIG 15 at offset 0;
“E:\SEQUENCE\export\EG_DB\991015a\991015a-047.scf’ (47>625)
GCACGAGGACATTCATGTCTACTTAGTTCCTTTTCTCCACCCAGTACCAATCACTTTAATTTTCCAAGTTGTATTAGCTTCCAGGCACCTGCATTTCAAGGTA
GCATGCTTTGCTGGCTCCCTACTCCCCGCTCCATCTGCAAGAATAGGTCTGGTACACTGTGAGTCCTTCTAAACTCCATGATGAATCACAGATACTGCCATAT
TCATGACATTGTAAATGGCCACCCCTAGTCACCATGTCAATGACCTCTTTGTCAAGCTTTCCGTGGTGTTGCTGACCKATTCAGCTCTATTTTAGATACAGTG
TCCCAGTCCTGTCCTCCAGCCAGGAACACCCCAAAGCCCACTGGTTAAATCTCTGCGTGCTCTCGAAACCTTACATTGTATTCAATTTTCCATTGCATCATTG
TCTATACATATCAATATCATGAAATCAATCCTGCTTATAAAGAACCATCTTTACATAACAGATATAAATTAACCTTCAGATTATACTGACTAAACAGAAAAAT
CCTGTCAGCCTCTTATTCATTGATCTTACGGGATACCTGTGTTTTCATTCTTATACTCAGCTTT
>‘991015a-048.scf” came from CONTIG 16 at offset 0;
“E:\SEQUENCE\export\EG_DB\991015a\991015a-048.scf’ (47>653)
GCACGAGGTTGCGGCCCACCTGGTGTACTGGGGCAAGGCCATCATCATCTACCCGCTGTGTGAAAACAACGTCTACATGCTGTCTCCCAACGCCAGCGTGTGT
CTGTATTCCCCGCTCGCCGAGCAGTTCTCACGCCAGTTTCCATCTCACGACCTGCCATCTGTCCTTGCCAAGTTCTCCTTGCCTGTCTCCTTGTCAGAATTCA
GGAACCCCCTGGCCCCCCCTGTTCAGGAGACGCAGCTCATCCAGATGGTGGTGTGGATGCTGCAGCGCCGGCTCCTGGTGCAGCTGCACACCTACGTCTGCCT
GATGGCCTCGCCCAGCGAGGACGAGCCCCGCGCCCGCGAGGACGACGCGCCCCTCGCCACCAGGGTGGGCGGCCGCAGCCTCAGCACACCCAAGCCCTCAGCT
NTGGCTCCCCACCAGCAGCGATGACATGACCCTACCAGCCCCAGCATGGACAACTCAGCGCTGACTGCTCCCCAGCGGGACTCGGCGCTGACAGGAGATGACG
GAGACCTGCTGCCCACCTGTCCGACACGACNGCCCGCATCTCACGGCCTCGGCCANAACCCGAGACCCCGATGCTGCAGCTCTGCCTATTCC
>‘991015a-049.scf” came from CONTIG 17 at offset 0;
“E:\SEQUENCE\export\EG_DB\991015a\991015a-049.scf’ (41>656)
GTAATCTTCACATTCATTTTAGCAGGTACTGAGTGATGCTGTATATATAAATAAGTGTATTGTTTGATTTTTAGACCACCACATGACATGCTTGACTATTATT
TCAAATGTCTGTTAATGCAAAGTAGGCTACTCCATAATAGTGTTAAGAACAAAACTTACTAACAAAGTGATATAAAGACTTAAATTAACACATTATGTGGAGC
CCTATCTTTACAAAAGTTTTCTACTGTAAAATGCTTTTACTTTTATTTGCAGTTTTCATTTGATAGTATTCACCATAATTAAAGTTGCATAAGATAATTGCTT
CACATTTCACATACCTATATTTATCTGAGTGCTGTCTAAAACTGTTGTGCTAGCCAAAGTAAGGCTATGAAATCATTTGCAGACTAACCCCGAGTTTGTGTTA
AAGCAGTGTTATTGCCATGNGAGAGGCATTGACTTTGTACCACATCATGTTCAACCATTTATACATTCAGGGCCTTTTTTAAAAAAGAAAAGAAAACAAAACA
ACAAATAGAAGAGCATTATTGACAAAACTTTTTTTTCTAACCTGCAAAAACTAATTTTCTGCCTAACGCCCTTACCCGACACCACCTTGAAAATGCATGAA
>‘991015a-050.scf” came from CONTIG 18 at offset 0;
“E:\SEQUENCE\export\EG_DB\991015a\991015a-050.scf’ (39>753)
TGCTTGTTGGGAGAAGCAGGTACTTATAGGGACGACGCAAGGTGCCGGATGTGAAGAAAAGCGGGGGCGTGGAGACTCGGACCCGAGGTGTGAAACAGATTCT
TTCTTTGCCTTGAGGATCTTATCTT7CCTGTGTGCATTCTCTGACGATTCGTTACCTACCTGGAGTGTCTTTGACTGTGGGCATCCCCCGGTGTTTTTTAAGG
CACTTAAGCTGCTCCCATGCTTGTTTGACGTCTGCTTTGTCGAGTGTCCATGATGTTGGGCCCTGAGGGAGGTGAAGGCTTTGTGGTCAAGCTCCGTGGCCTG
CCCTGGTCCTGCTCTGTTGAGGATGTGCAGATTTCCTTTCGATTCACGATCCATGACGGGGGTGGGGGTGTTTCTTTATTTACACTAGAAAAGGAGGGAGAGG
GGGAGGCTTTTGTGGTCTTGATTAGAAATGACTATATTGGCCCTTAAAAAACAGGAAGCTGGACATCGTTCTTGAATGTGAAGCCCAAAATCGAATGTTGGGG
TTTAGCAAGGGCCAAAGGGTATGTGCCTGAGGTTTGGCGTTCGGGTTCCGTGGTGCCCCGGAATTATTTTTTTTTTGGGGGAATGGCCTGGTTCTTTCGGGCC
CGGGCCATTCGGGAGCTTGTTTTGTCTTCGATTTTAAGGTTTGAAACAGGATATGGTCTTTGGGTTAGGTCGATATTTTTTGTCCCCTTTTTTTTTTC
>‘991015a-051.scf” came from CONTIG 19 at offset 0;
“E:\SEQUENCE\export\EG_DB\991015a\991015a-051.scf’ (41>646)
TTCTCAACCAACCATAAAGATATTGGTACCCTTTATCTACTATTTGGTGCTTGGGCCGGTATAGTAGGAACAGCTCTAAGCCTTCTAATTCGCGCTGAATTAG
GCCAACCCGGAACTCTGCTCGGAGACGACCAAATCTACAACGTAGTTGTAACCGCACACGCATTTGTAATAATCTTCTTCATAGTAATACCAATCATAATTGG
AGGATTCGGTAACTGACTTGTTCCCCTAATAATTGGTGCTCCCGATATAGCATTTCCCCGAATAAATAATATAAGCTTCTGACTCCTCCCTGCCTCATTCCTA
CTACTCCTCGCATCCTCTATAGTTGAAGCTGGGGCAGGAACAGGCTGAACCGTGTACCCTCCCTTAGCAGGCAACCTAGCCGATGCAGGAGCTTCAGTAGATC
TAACCATTTCTCTTTACACTTAGCAGGAGTTCCTCATTTTTAGAGCCATCACCTCATTACACAATATCAACTAAGCCCCCGCATGCCACATACAAACCCTCGT
TGTATGACGAATATTACGCCGCCTATATACCCGCTCCTGATACAGCCGTCCAAGCTATACAGACGACCAAAACACTTTCGACCGCAGAGAG
>‘991015a-052.scf” came from CONTIG 20 at offset 0;
“E:\SEQUENCE\export\EG_DB\991015a\991015a-052.scf’ (47>749)
GCACGAGGCTCCCAAGGGACGGGCAGCCATGCTTGTATTTTTATGGTTAGAAAGGCACAAAATTATCAACTAAGACATTCCTTCTTTCTCTTTTTTTCCTGAA
CATCATGGAGTTTTCCAGTTGTCTCTTTTGGACTGTAGTTTTTAGTGTTTTAAACAAACACTTTACAATGTAAACTATTTATTTTTTACTTATTCTGGGGGAT
CTGTCTGAAAGACTATTCATGGAACAGGAAGAAGCGTAAGGACTATCCATATCATCTTTGCTACAAGTCATTATGACTGTAAGATTGTAAATACAGATTATTT
ATTAACTCTGTCTACCTGGAATCTAGTTTCATAGGAAAGTGTTTGAGAGCAGGTAAGTTGAGATCGATCAGCAAATCTTTCACAGGAATGGCACAAGGAAACC
AGCATAGCAAGCTGCTCTTACCCTGTGCTAGACTGGATGATTTGGATTCTTTTTTCTTTTTTTTCCCAATGGATTACTGGTTGCCCTTGCAGTGTTTTAGTTG
CAAGAAAGCAAAGCCTTATACTGCTATCCATCCTGTGCTATTTCAGGAGAAGAAACACTATTTATATTTTCTTTTCAAAAAAAAAGAAAGAGACTGTTACAAA
TTCCTTTTGTGGGACGAAAAAATTGATAAAAAAAATAGGGTACAATTAATCGGAACAAAAAAAAAAAAACGGGGGCCCCCCTCC
>‘991015a-060.scf” came from CONTIG 21 at offset 0;
“E:\SEQUENCE\export\EG_DB\991015a\991015a-060.scf’ (42>635)
CGAATGGTGTGTACACTTGCACTTTCATCCCAGCAGAGGGACCATAGCTTTCATGATGTTCTTTAAGACAGCTGCGCCCCACAGCTGCCAGGTCAGGGCAGAA
TGAGTACCCAGGAGGAAGGGCCTGCGTGTGACCCTCAGGCACCCCACCCACCCCTCCTGGTCCAGCCCACCCCCACTGGCTTTAACAAGTGAAAGTGAAAGTC
GCTCAGTCATGTCCAACTCTTTGCGACCCCACGGACTGTCCAGTCCATGGAATTCTCCAGGCCAGAATGCCGGAGTGGGTAACCGTTCCCTTCTCCAGGGGAT
CTTCCCAACCCAGGGATTGAACCAGGTCTTCTGCATTGCAGGCATATTCTTTACCAGCTGAGCCACCAGGGAAGCCCAGCTTTAAACAATTAGTTTCACCAAA
ACCTTTAGCTCCAGGAGGCGGCTTCTGTTTTGGNGCTGTGCAGCGACCCTCCTGGCTCCTGACANGGACGGACGTGCACGGGCTGATTGACAGGTCACTGCAG
GCTTCAGAACCGTCACCAAAAGCCCAAGACCTGAAAGAGTTGGGCTGATTGGGGCTCCGCAACGGGGATGGCCTACCGC
>‘991015a-061.scf” came from CONTIG 22 at offset 0;
“E:\SEQUENCE\export\EG_DB\991015a\991015a-061.scf’ (48>697)
GCACGAGGCTTTTTTTTTTTTTGGTAAGGTTGAATGCACTTTTGGTTTTTGGTCATGTTCAGTTGGTCAAAGATAAAAACTAAGTTTGAGAGATGAATGCAAA
GGAAAAAAATATTTTCCAAAGTCCATGTGAAATTGTCTCCCATTTTTTGGCTTTTCGGGGGTTTCAGTTTGGGTTGTTTGTCTGTTTCCAGAGTCAGGGGCAA
GTGGGTTGGGTGGGAGGGAGCCAGGTTGGGGTGGAAGGAGTTTACAGGAAGCAGGCAGGGCCAACGTCGAAGCCGAATTCCTGGTCTGGGGCGCCAACGTCCA
AGGGGGCCACATCGATGATGGGCAAGCGGGAGGTCTTGGTGGTTTTGTATTTCGATCACTGTCTTGCCCCAGCTCCGGGGTGACTCGTGCAGCCATCGTAGTG
ACGCTGTAGGTGAGCGGCTGTGCCCTCGCCCGATCTCGATCTCGTTGGAGCCCTGGAGAGCAGGCCTCTTGAGGTGCAGTTGCTGGTCTGTAGCCAGCTTTTT
GCAGGTAGGGAGTTTGGGAGCTCGNGGAACAGCGCAGAAGCAGTGGAGCAATCGAGATCGACCCGGCGCTAGCGACGATCGCGCAGCTCGCGACAACCGCTTT
TTTGGTTTTTGTACCATTTTGCCCCCGCGGGGG
>‘991015a-069.scf” came from CONTIG 23 at offset 0;
“E:\SEQUENCE\export\EG_DB\991015a\991015a-069.scf’ (48>644)
GCACGAGGCTCTCGTGTAAGGACTGACTACCTGGACCGATCGCCTGACAGATCCCACCTGCCTGCCCACTGCCATGACTGAGCCCAGCCCCAGCCCGGTCCAT
TGCCCAGCATTCTCTGTCTCCTCGTCGGTCTGTTCCACCACCTTCAGGGTCTTGGTTTGTCCACTCGTGTGTGACCTTTAGTCTCTAGGCTTTACCAGAAGCA
GTCTGGGTTCAGCCAGTCAGTGACTGGCGGGTTTGAATCTGCACTTGTCCCCACCATCTGGGGACTCCCCTTTCCCCGTGTTGTCCAGGACTCCCCATGTGTC
AGTGCTCTGCCCTCACCCTGCCCAAGACTCACCCCCCTTCCCCTCTGCAGGCCGACGGCAGGAGGACAGTCGGGTGATGGTGTATTCTGCCCTGCGCATCCCA
CCCGAGGACTGAGGGAACCTTGNNNNGGGACCCTGNNGCTGGGGTGCCCTCGTGATCTCCTCGCCCTGTATTTTCTTCATCTNCAGNTCTGGACAGGCAGNGG
GCCAGAAAAGGNACCTANTTACCATTGCGNGGAGATGAGTCATGGAGGTCAAGGGAGACGACTCTGATTTCCAGACCCCCTC
>‘991015a-077.scf” came from CONTIG 24 at offset 0;
“E:\SEQUENCE\export\EG_DB\991015a\991015a-077.scf’ (42>659)
TCGAGTTTTTTTTTTTTTTTTTTTTTTTTTTTTTTTTTTTTTTTTTTTTTTTTTTTTTTTTTTTTTTTTTAGGAGCCGCATCAATAGAAAAAAACATAAAGGA
AAAGGCAAACTACGACTACGAAAGGGCAGAATCAGGCAGGGGCTTCAAAGCCGAAGGGGGGGGTAAAAATAAAAAGAAATTTTAATGGGGGGAAGAAGCAGAC
AATTAAGAAGGGGGACCCAAGGGGGACGGGGGGGCCGGGGAAGCCGGGGGCTACAAAAAAAGGGGAGCCGAAAACCCCGGCGGAGATAGGAAAAGGGGCTTCA
TAGTATTTTGAGGCTGGGAGGAGTGGGAAGGAAACCCCTAAGGGATGGGGAAAAAAGGGGCTGGAATATAGCTTTCGGTCCCCTTCTATTAAACATGAGGGGC
CCAGGAAAAAAAACTCCGGAGCCAAAGGACAGAGGGGGGGGCAGGGGGACTTCAGGGGGGGAGGGGGGAAGCCCGGGGGGGGCAACCCCGCTAGTCGGGGGGG
GGGGGGCTGGGGGGAAAAACCAAAAAACCGGAAAAAAAACCTGGGAAAAAAAAAATATTCAAAAGGGGCTTTGGGCACGGGGGGGGGCCTGGAGGGCTTCTGG
T
>‘991015a-086.scf” came from CONTIG 25 at offset 0;
“E:\SEQUENCE\export\EG_DB\991015a\991015a-086.scf’ (44>139)
TATTCGGCACGAGGGGAAAAAGTCATGTGATAAATACTCCTTTTGTTCTATAACCAAGCCCCAACCAAGGACAAATTATGATATATACTGCAGGGG
>‘991015a-087.scf” came from CONTIG 26 at offset 0;
“E:\SEQUENCE\export\EG_DB\991015a\991015a-087.scf’ (44>525)
CGCTCTCAAGGGGCGGGCGGGGCTCCAGTTGGGTGCTCCTAGAGCGGTGCCGGAAAGTGTGCACAGTCTCGTCTGTGTGCGTGTGGATAATATCCTGTGTCAT
AGAAATCTGGCTTCATAAAACCATAGAATAAGTTTCTGGTATGAANGGTCCCTTCTCTTGTAGCTCTTCACTTGACGCCACAAGACCATACCCAAGTGGGAGT
TCGCTGATCGAAAAAGCACCTTCTTGGTAGAACCCGGGAAGGAACACCACGCTCGCCTGAGGGCCCTGAAGGGTCTCCTCTATAAAGCGCTCACAGACTTGGT
GTGCACCCCTGAAATGAACCAAGAATTGTGCGACTTGAACGGGGAGGTGTGGAGGGCCGGTTTGACTTCAGATTTCTCTGCTGCCGTGTTCTTCTGGAGGGCG
ACCCTTTCCAAACCTCAGAACGACACACAAGGGGCGGCCTGCGAAGAACGCTGCGGAATGAAGCATCTCT
>‘991015a-089.scf” came from CONTIG 27 at offset 0;
“E:\SEQUENCE\export\EG_DB\991015a\991015a-089.scf’ (50>609)
GCACGAGGATACAACAGCTTCCAGATGCACTTGGACAGCCAACAGTTTTTGAATGACCTGAGAAATGATATTGAGAAGAAAATAGGATTTGATGCTATTATGC
GGGTTCGCACCAGCACAGGTTTCAGAGCCACTGACTTCTTTGGGGGCATTTATATGAACAACACCACCGACGTAGAGATGGCAGGAATCGACTGTGACAAGGC
CGTGACCGTGGAGTTCAAGCATGATGACAAGCTCAGTGAAGACACTGGAGCCTTGATTCAGTGTGCCGTGCTCTACACAACTGTCAGCGGTCAGAGACGACTT
CGGATTCACAACCTGGGCTTGAACTGCAGCTTCCAAGTGCGGGATCTGTATAAGAGCTGCGAGACAGATGCCCTCATCACTTTTTTTGCAAGGCAGCTTTAAA
GGCGTTTTTACCAACCTTGAAGTCATTGGGAAATTTTGGTTACCAGACTGNCACATGTTGGCTGTTACGGAGACTGGCTAGCCCTCGCACCGCCAGTGATTTG
CGATTCATAAGTCTGCGNGTACTGACTGTTGTGAGACTGGTGTGT
>‘991015a-095.scf” came from CONTIG 28 at offset 0;
“E:\SEQUENCE\export\EG_DB\991015a\991015a-095.scf’ (46>593)
TTCAGACAGCTGGTGCCGAAGTTTCAGGAACATCCTCCTCTCTCCAATCATGGCTTGTGGTCTGGTCGCCAGCAACCCTGAATCTCAAACCTGGGGAGTGCCT
CAGAGTGCGGGGCGAGGTGGCCGCAGACGCCAAGAGCTTCTTGCTGAACCTGGGCAAAGACGACAACAACCTGTGCCTCGACTTCAACCCTCGTTTCAACGCG
CATGGGGACGTCAACACCATCGTGTGTAACAGCAAGGACGCTGGGGCCTGGGGGGCCGAGCAAAGGGAATCTGCCTTCCCCTTCCAGCTGGAAGTGTCGTGGA
GGTATGCATCCTCTTAACCAGACGNACCTAACCATCAAGCTGCCTGAGGATACGAATTCAGTTCCCCAACCGCCTCACCTGGAGCCATCACTACCTGCTGCAN
GTGNGGACTCAGATCNGAGGGGTGGCTTTGAGGACTATGGCCAGCAGCCCTGGCCCCATAAGGAGTGCCTGGCTCCCCTGAAAAAAAAAAAAAAAAAAAAAAA
AAAAAACTGAGGGGCCGGGACCCATCCGTTAAG
>‘991015a-096.scf” came from CONTIG 29 at offset 0;
“E:\SEQUENCE\export\EG_DB\991015a\991015a-096.scf’ (43>550)
TGGAATACACAGCTGGGGCGGTGATGGCGTGCACTAACGGTTGTTTGTCCTACCCTTTTGGGCCAAGGGAGGGACGCAGCCCCGTCAAGTATCCGCCCGCTTT
CAGCCTTACTGCCTTTTATGCCACGGAGCCCCCGAAGGGCCGAAAGCCGGAGGGCCGCGGCCCTGGGCCCCCCGGCACCCTGTGCAGCAGGCTTGCCTGAGCC
CGGGTTGGGCTGGCGGCTTCTTTCTCTGTGGGCTCCCGGTCCCGGTCGGGTTTTTCAGGCCGCACCTACCGGCGGAGGAGGGATGGGGGACTTTGGGTGGCCC
TTTTTCCAGTGACGAGGTAGCTGACGATGGCGTATGCCATATGTGGAAGGATGAACATCTTTCCGAATTTACATTAAAAGATTAAAAGGATTGGTGGGAGGGA
TTCAGCGCCCCCTCATGTCCCCAGGAAGTGCCCCATATACATGGGGGCACCCCAACGGGAATCTGGGGATGGCTGGAACCGGGCCCCGGTGCTGGG
>‘991108a-001.scf” came from CONTIG 1 at offset 0;
“E:\SEQUENCE\export\EST_db\991108a\991108a-001.scf’ (58>663)
GCACGAGGGTTTAAAAGACCGAGTCCAGTATGGGGGGGGAAGTGAGAGGCAATGGAGAAAAGCCAGAGGTCAGGAAGACAGCTCACGAGGAGGGAGGAGAGGT
CTGCCTGAGCTCTGCAGATTCCTGGAGCAGGGGGAATGGAGAGGCAGCCAGGGGACCCCTTATAAACAAAAGATGGCCAACACTGGTCAACACCACTGGAAGG
GAAAGCCCCGGGGAACACTCATCTTCACTTTCAAAACATGGGTAGAAGACCATTGTGAAAGATATTAAGAAGAACCGCTCAATCAAGGACATTACTAATATTA
CCATGTAGATGAATAAAAACAAACTGAACAGATCTAATGGAGATCCTTGGCCTGGCTTTGAAGAGATGGTTTTAAATTCCGGGACACCATTCCTCCTGNACCC
ATACTCTCCAGTTAGGTTAGCTGAAAGGAAAAATGCCTCTCCTAGAAGGGAGGCATAACACAACCTGGCGGGCAGCAGAATTTCCCTGAAAAAAAATTACATT
GCTGTGCTAAAAATCACTTCTTTGTTGTTCTCTCCAGCCCCCCCGCCACGCCGTTTAAGGGGAGAAAAATGAAAAACAAAAATTAGCGTTT
>‘991108a-002.scf” came from CONTIG 2 at offset 0;
“E:\SEQUENCE\export\EST_db\991108a\991108a-002.scf’ (53>440)
ATTCCTTAGTCTTCTCATTTCTTGAATCATTTTCATTCTTCCTCAGCACTGCAGCATCCTTTGCAGCATTCATTAAGGGGTTTCCTTTAAATACAACAGAAAG
AAAAAAAAATTCAAACAAAAATTGAGTTCTTTCAAAATGAACTAAGTACAGCTCATTATAGCTTCCCTCCAGTTGTTTGGGGGAAATTGACTCCAGGTATTTA
TAAAACAACACCCATGGAAGCAAAAAAAGCTGAAGCTTGAGAGAACCTGATAAGGGAGAAGTTACAAAAGTTATCAGAACAGCTTTTGGCCTTTTGCACCTGG
GAAATTAAAGAGCAATCCAGGGGGGTTGGGAGAGGGCTGGATGGGACAAGGGCCCCACCTGGGGGGCACTGGCCAGGGG
>‘991108a-004.scf” came from CONTIG 3 at offset 0;
“E:\SEQUENCE\export\EST_db\991108a\991108a-004.scf’ (57>453)
GCACGAGGGTGGAGTTCGTCTCCGCACGCGTGACCGCGCTGGGATGGATTCCTCCTCGTCTTCCTCCGCGGCGGGGTTTGGGCTCGGTAGACCCGCAGCTGCA
GCATTTCATTGAGGTGGAGACTCAAAAACAACGCTTTGAGCAGCTGGTGCACCAAATGACGGAACTTTGTTGGGAAAAGGGCATGGACAAGCCTGGGCCAAAA
GTGGACAGGCGGGCTGAGAGCCTGGTTTGTGAAGTGCGGTGAACGCTTCATTGACACCAACCAAATCKITJTGAATCGACGGAACAAAACCACAAATCCAACC
AAGCCTCTCAAAAAGCTTTCTGACTGACTCACATTATCTTTTTGTAAAGAAGGAGATCAAGAAATGAAAGCAGTGATGGGGAGTGTAG
>‘991108a-005.scf” came from CONTIG 4 at offset 0;
“E:\SEQUENCE\export\EST_db\991108a\991108a-005.scf’ (58>458)
CACGAGGCAGGCTCTAACCCCCTCACATTCCTGGCGAACCCTCCATACTGCCCCGGCATCTTTGGCCTGCCTGCCTGCCCTGCCCTGTCCACTTGGGGGTTTG
CTCCAGTATCCTATGTTGGGCTCTTGGGATCTTTTCTTTTTTCCCTTTGTCCTTGGGCCGGGAAGGTCCCCTTGGGCATGTCCCCTTCAACCAGGTTAAGCTC
TTGGCCTTGATGGATGACTAGAAGGTGGTGTGGATAGAAAACCCGTGGTCCGATGGGGGGCCGTGGTTACCCTGTGGGTATGCATATCCCCGGTCCAGTGGGT
TAGTTTAGTAGATATTTCGAATGATGTGTTCTAGATGCAAACCGTACACAAGGAGGGGTGCTGGGCACGAAATACCCCCTGCCAAATCCCAC
>‘991108a-007.scf” came from CONTIG 5 at offset 0;
“E:\SEQUENCE\export\EST_db\991108a\991108a-007.scf’ (1>610)
CACGGGGGGCCGTTAAATATGGTCCCCCGGCTGAGAATTCGCCGAGCAAAGCCACAGGTCTACGTTGCCCCCAGTCCTCACAGCCGCTCTACCCAAGGGCCCC
AGTTTGGGGGCATCACAGCTGTCACCGCGAACCAGAGCCTGCTGAGCCCCCTCAAGCTGGAGGTGGATCCCAACATCCAGGCCGGCCGCAGCCAGGAGAAGGA
GCAGATCAAGACCCTCAACAACAAATTTGCCTCCTTCATCGACAAGGTGCGGCACCTGGAGCAGCAGAACAAGGGTCTGGAGACCAAATGGAACCTCCTGCAG
CAACAGAAACTGCCCGGAGCACATAAACAACATG1TTGAGAGCTACATTAACAACTCCGGCGGCAGCTGGAACTTTGGCCAGAAAAACTGAACTGGAAGTGAA
GCTGGCACATGCAGGGCGGGGAGGACTTCAGACAAAATGAGATAAAACAAAACGCCAGAATGAGATGATTTGTATATCAAAAGATGGGTGAACTAATGACAGG
AAAATGAGTCGCCTGAGGCGATGAGAATAAATCTCAGCACGGTGAAAGAATCGAAAGAATCAATTTTAACTCGGCCGTCTGACAACCCACCGACT
>‘991108a-009.scf” came from CONTIG 6 at offset 0;
“E:\SEQUENCE\export\EST_db\991108a\991108a-009.scf’ (54>586)
GCACGAGGCAGGACGTCACCTTGCACGACTTGGACGTGGCCAACGCACGGCCCCAGGGGGGACAAGACATCCTGTCTATGATGGGCCAGCTCATGAAGCCCAA
GAAGACGGAGATCACAGATAAACTGCGGGGGGAGATCAACAAGGTGGTGAACAAGTACATCGACCAGGGCGTGGCCGAGCTGGTCCCGGGCGTGCTGTTCGTG
GACGAGGTCCACATGCTGGACATCGAGTGCTTCACCTACCTGCACCGCGCGCTCGAGTCCTCCATCGCGCCCATCGTCATCTITCGCGTCCAACCGCAGCACT
GCGTCATCAGGGCACGAGGACGTCACCTCTCCTCACGCATCCTCTCGACCTCTGACCGAGGATGATCATCCGGACCTGCTGTACACCCGCAGAGATAAACANA
TATAAAATTCGAGCCANACAAGGGATCACATCAGGAGAAGCGCTGACCACTGGGAGATGGAACAAACCACTGAGTACCGGCACTGCTGCCCGCCACTGTGGCA
GATACGGAAGAGGATGAGA
>‘991108a-011.scf” came from CONTIG 7 at offset 0;
“E:\SEQUENCE\export\EST_db\991108a\991108a-011.scf’ (55>597)
GCACGAGGGGAAGGGAGATTGCTTTCTCCTACTGCATCTGTTAATCCTTTATCCAGAAGTCCCCCTGAAACTTCTTCACAGATGACTCCTAATCCATTACTTT
TAAGTCCTACCACAGAACTAATGGAAGAAATTTCTGAATCTGTTGGAAAGAATCAATTTACTTCTGAAAGTACACATTTGAACATTGGTCATAGGTCTATGGG
TCATAGCATGAATATTGAATGTAAAGGGATTGATAAAGAGCTAAATGATTCTAAAACTACACATATAGATATTTCAAGAATAAACTCTTCTCTGGGAAAAAGC
CAAGTTTGACTTCTGAATCCAGTATTCATACAATTACCCCTTCAGTTGTTAACTTCACTAGTTTATTTTATAACAAGCCCTTTCTGAANCTTGGTGCAGTATC
TGCATCTGACAACACTGCCAGTTGCTGANAGCCTAGCACTACTTGCAGTCCAACCCCTANAAAAAAAAAAAAAACGGACGCAGCCACTCACCGGGCCCGCTGT
CCTGCTCGCCAGACTTCTACACTATGNC
>‘991108a-012.scf” came from CONTIG 8 at offset 0;
“E:\SEQUENCE\export\EST_db\991108a\991108a-012.scf’ (55>244)
GCACGAGGCCCAAGTGCAGGAGAAGCAGCACCCAGTGCCCCCACCGACTCAAAACCAAAACCAGGTGCGCCCGGCTGGGGGCCCCGGGGGCCTCTGACACTGA
AAGAGGTGGAGGAGCTGGAGCAGCTGACGCAGAAGCTGATGCAGGACATGGAGCATCCTCAGAAGCAGAGTGTGCCCATCAACGAGT
>‘991108a-058.scf” came from CONTIG 8 at offset 0;
“E:\SEQUENCE\export\EST_db\991108a\991108a-058.scf’ (53>714)
GCACGAGGCCCAAGTGCAGGAGAAGCAGCACCCAGTGCCCCCACCGACTCAAAACCAAAACCAGGTGCGCCCCGCTGGGCCCCGGGCCTCTGACACTGTAAGA
GGTGGAGGAGCTGGAGCAGCTGACGCAGAAGCTGATGCAGGACATGGAGCATCCTCAGAAGCAGAGTGTGCCCATCAACGAGTCCTGTGGCCGGTGTCATCAG
CCCCTGGCACGTTCGCAGCCCGCGGGTCGCGCTCTGGGGCAGCTCTTCCACATCACCTGCTTCACCTGCCGCCAGTGTGAGCAGCAGCTCCAAGGCCAGCAGT
TCTACAGCCTGGAGGGGGGCTCCGTACTGTGAGGGCTGCTACACCGACACCCTGGAGAGTGCAGCACCTGTGGGCAGCCGATCACTGACCGCATGCTGAGGGC
CACAGGCAGNCCTACCACCCGCATGCTTCACCTGTGTGTCTGCGCCTGCCCCTGGAGGCACCTCTTATTGTGGACAAGCCACCGCCCACTGGCCCCGACTACA
CAGCATACCCCCCAAAGCTTGGGCGCGGACCATATGCGACCTGCCGCGGGAGACGCGCGGGGCTTGACAGACTCCATAAGGTAAGGGCAGACGGGGAGCCTTC
ATGAGGAGAACGCGTTCCTGGTGCCCGTTGGGGAGCCACCCGAC
>‘991108a-013.scf” came from CONTIG 9 at offset 0;
“E:\SEQUENCE\export\EST_db\991108a\991108a-013.scf’ (55>115)
GCACGAGGCAACTGTCAGAGAAACATGATGGGGTGAAGGTGGGTGCTTTTTTATAAATCGT
>‘991108a-014.scf” came from CONTIG 10 at offset 0;
“E:\SEQUENCE\export\EST_db\991108a\991108a-014.scf’ (53>744)
GCACGAGGACCACCACACAGCCCCTCCTTTCTGATGGCTCTCAACTCTCGATCCCACAGACTCACACCTCTGAGACCTGCAAAAGAAGCTCGCGGGTTCAGTC
CCCCAGGGGGGGACCAGGGTTGTGGGGGGGACCGTTGGATATGTTGGAAGCGGTACTCGTACCGGCTTCACATTTTGTTGTCAACAATTTACTGTATTTTTTT
TTTTTTTTTACTTTTTCTGTACCAGTTTTGCTATAATTTATCAGAAGGTCCAAAAAGTTCGACATAACTATTTCAGTTTTGCATTATTTATTTATGATGCTTT
TGTCATTGTTTTTTTATACATTTGGGATTATAAATTATGTAAATGTTAAAATGAGCATCTCAAAGAAGTCTGTTAAATCATGGCCGGGGTGGGGGAAAAAATA
AAAAACAGTTTTATTTTTAAAAAGGGGGACCCCAGTTTTAATGATCAAAGTTATAAATCAGAAATCCTGTAACCACTTCCTAAAAAAAAGGACCACAAATAAC
CGATTTTTTTATTACTCCTTGCCCAAGGGGCTCTATGCGACACCCTTTTTTAAAAGGCGCTCTTTTTTAACAACTCTGTTCGCTTGCCCCGGGACCACAGGTT
CTTCGGTCTACCAAAATATTAATTTTTTCCAAAACAACCCGCTGTTTTTGACCTTACCTATTCTTTTTGCTGCCA
>‘991108a-015.scf” came from CONTIG 11 at offset 0;
“E:\SEQUENCE\export\EST_db\991108a\991108a-015.scf’ (49>678)
TGGCTCCGCCACGAGCTCTGGGACCCGGGTCTGTGGCCGGCCGCTCGCTGGCCCTGTCTCCCAGCGACGGGCAGCTACTGGGCCGGGGATTCTGAGCTTTGCC
TGCTCTCTTTGCACGTGACATGATGTCCCCCCTCCACAGCTCCTGGAGGACCGGCCTGCTGCTGCTGCTGCTCTTCTCTGTGGCAGTCAGAGAATCTTGGCAG
ACAGAAGAGAAAACATGCGACCTGGTGGGAGAAAAGGGTAAAGAATCAGAGAAAGAGTTGGCTCTCCTGAAGAGGCTGACACCGCTATTTAACAAAAGCTTTG
AGAGCACCGTAGGCCAGAGCCGAGACATGTACAGCTATGTGTTCCGGGTGTGCCGAGAAGCTGGCAACCACTCCTCTGGGGCAGGCCTGGTGCAGATCAACAA
AAGTACGGGAAGAGACGGTAGTTGGGAAATTCACGAAACTAAATCTCATGGAGGAATGGATCAGCTGACTATAAAGGGGGATGAATAGACAACACGTGGCAGG
AGCACGGGGGCAGGGGAGATCTCCGCACGACCACCTACGACAATTACCGGTTGAGACGACCAAACCAAATGTTTACCTTTAAGAAGCGCCGGGGGCCCAAACC
CCCCTAGGGGT
>‘991108a-016.scf” came from CONTIG 12 at offset 0;
“E:\SEQUENCE\export\EST_db\991108a\991108a-016.scf’ (48>563)
TTTTTATATTAGTTTTGTAATCCCTACATTGGAAAGCTTTCCAAATTCTACTTGCATTTAAATAAAACTGTTGCAGTTTTTPACTATTTATTTTGTTTCCCAT
GGTTTAAGAAAATAACTGCACAGTTTCAAAGGCATGGAAAATTATATCAGCCTTTATGTACTCTGTTCCCAAAATGGCAGGGTCTAGAGAAGAGCAGAATTCC
AGCTTTAGAAAACATTCTAAGATTTCACGCATGCAGTTMTGACATATCTGAAAATAAGACTTTTGTATATTTGTGGTGGAGGNGGNTGGGGAAACTTTTACAA
AATGNTNNATTTTTGTCAGTCTGTGGGCATTTCACATATTTTTATTGCATTAGATTTGGTATTATGTCGACATTATATATTATTACTTCTTGTTTGATTTGTG
ATTCCCTATACTITGATTTTTTAGTAACTTTTTTATAAAGAACTGCTCTTTTTTTTTAATTGATTGCGCTCAGATGAGCGCGAGTGAAAAGCAACATCAATGA
CAT
>‘991108a-017.scf” came from CONTIG 13 at offset 0;
“E:\SEQUENCE\export\EST_db\991108a\991108a-017.scf’ (54>489)
GCACGAGGCTTAAACAGAAGACGGGCCCAGGCATTGTTGAACTGTCTCGTGCCCTCTCCTTGGATCTTGGATCTTGGTTTCCTCGGTAGGAGTTTCTGTCCCA
GAGGCATTCAGGTGCATTTTTTTTTTCTCCTCCGGTGAAGGAGGTTCCAAACCTATTCTGGTTTTTTTCCTACCTTGTTGTCATTGTATCTCTCCTTTCCTTT
GTTCTCTTTTATGTTTTTTTTTTTCTTTTCTTTTTTTGGTTGTTTGTTTTTCTCCATCAGTGGGGACTGATTGTTCCCCTTGCCGGCCAAATTTTGTTCCTTC
CCCTGTTTTGGCCAAATCCTAGGGGNGNAAAATCCTGGTATGCCAAAAATATATGCTGAGCATAAGTCATTCCACGTGGGTLTGTCCATCGCAGCCGAGAAGC
TGCAGNGGGGGCAGGGAGNNGGCGC
>‘991108a-022.scf” came from CONTIG 14 at offset 0;
“E:\SEQUENCE\export\EST_db\991108a\991108a-022.scf’ (54>474)
GCACGAGGGCGTGGTCCACGCCGAGCGATAGAGACGCCTCGGCCGTGTCTTCAGGATGACGGAGTGGGAAACAGCTGCACCTGCAGTAGCTGTAGACCCCCGA
CATTAAGCTTTTTGGAAAGTGGAGCACCGATGACGTGCAGATCAATGACATTTCTCTGCAGGATTACATTGCTGTCAAGTATAAGTATGCCAAGTACCTACCC
CACAGCGCGGGCCGCTATGCGGTCAATCGCTTTCGTAAGGCACAGTGCCCCATTGTGGAGGGCCTCACCAACTCCATGTTGTTGCTTGGCCGGAACATTGTCT
AGAAGCTTCATGACCGTGCGCATCGTCTAGCTCGGGTTTTTTATCTTCCATTTTCTTACTTGTCTGATCTCCCTTTGTTCCTGTTGATCGTCTTTCTTAACTG
GTGCCCCGT
>‘991108a-026.scf” came from CONTIG 15 at offset 0;
“E:\SEQUENCE\export\EST_db\991108a\991108a-026.scf’ (48>461)
CGCCAGGACACAGGTGTCGTGAAAACCACCGTTAAACCTAAACCAAAATGGGAAAGGAGAAGACCCACATCAACATCGTTGTCATTGGGGCACGTAGATTCAG
GGGAAAGTCTACCACGACTGGCCATCTGATCTACAAATGTGGCGGGGATCGACAAGAGAACAATTGAAAAGGTCGAGAAAGAGGCTGCCGAGATGGGAAAGGG
CTCCTTCAAATATGCCTGGGTCTTGGACAAACTTAAAGCTGAACGTGAGCGCGGGGATCACCATTGATATTTCCCTGTGGAAATTTTGAGACAACAAAGACTA
TTTTTCCATCATTGATTGCCCCAGACACAGAGACTTCTTCTAAAACATGATTATCAGGACATCCCAGCTGACTGTTGCTGTCTGCTCGGTTGGTGTTGTGTTG
TG
>‘991108a-040.scf” came from CONTIG 16 at offset 0;
“E:\SEQUENCE\export\EST_db\991108a\991108a-040.scf’ (54>397)
GCACGAGGCAGGACCTGAAGGATCACATGCGAGAAGGCTGGGGACGTCTGTTACGCAGATGTGCAGAAAGATGGAATGGGGATGTTGAGGTATCTCAAAAAAG
AAGACATGGAATATGCCCTGGGTAAACTGGATGAAACCAAATTCCGCTCTCATGGAGGTGAAACATCCTACATCCGAGTTTATCCAGAGAGAAGCACCAGCTA
TGGCTATTTCACGTCTCGGTCTGGGGTGGAGGGGCCGGGACTTTCTCCTCCAAAGCAAGGGGTTCCCACACCTCTTTTCTTCTTTTCTACCCTACTGAACAGN
GGGTGGGGTTTTTTTTTTTTTTTTTTAGTGGACT
>‘991108a-027.scf” came from CONTIG 16 at offset 317;
“E:\SEQUENCE\export\EST_db\991108a\991108a-027.scf’ (52>486)
TTTTTTTTTTTTTTTTTTTGTAGAGCCGGGAACTTAGGGGCCCACGAGAAGCGCACCAGCCACCGTTCGTTTTTTTTCATCACCGGATAACACCCGGGGAGGA
CCGAGGTTAATAGCTTGGTGAGCATGCATCTTTAACCAGGATCAACGGTNGACGGAACGATCACCCACGGATACTCCGGTGATAGATGGGGATGAGGACATTA
CCTTCTTTAAAGAGGTACCATTGTTTCTGACCCACTGTTAACCTTGATTAATTGCGGACCTGTGTTACAGATGAGACACGATTTATTTCTGCCTTTGAACACT
ACTTGAGCTGATCCTTTCTTCTTGTGGGGCGAGCAGGTAGTACAATGCTCTGGGAAAGTGAAGGGTCCTAGCATGCGTGTNGTGTCGTCTATTCTTCTCAATA
ATCTGAGTTTAGATATAGCGGGGC
>‘991108a-031.scf” came from CONTIG 17 at offset 0;
“E:\SEQUENCE\export\EST_db\991108a\991108a-031.scf’ (48>385)
CTGGTGTGAAGATCAATCCCAAGAACTACACTGATAATGAATTGGAAAAGATCACAAGGAGGTTCACCATGGAGCTGGCCAAGAAGGGCTTTATTGGCCCTGG
CGTCGATGTGCCCGCCCCCGACATGAGCACCGGCGAGCGGGAGATGTCCTGGATCGCCGACACCTACGCCAGCACCATAGGACACTATGATATTAATGCCCAC
GCCTGTGTTACTGGTAAGCCCATCAGTCAGGGTGGAATCCACGGACGGATCTCTGCTACCGGCCGGGGAGTGTTCCATGGGATTGAAAACTTCATCAATGAGG
CTTCTTACATGAGTATTTTATGAATGACA
>‘991108a-032.scf” came from CONTIG 18 at offset 0;
“E:\SEQUENCE\export\EST_db\991108a\991108a-032.scf’ (53>555)
ACCTCATTCTGACAAAAGATTAAATCTTGTCAGAATGAGGTGATAGATAAAATCAAATGCGCGTGATTTAAGACGCAATCTCACTTTGCAGGAACGAAAGCTC
TGGCGATATCTTCGCAGCCGACGTTTTAGTGATTTCAAATTTCGCCGTCAACATCCAGTGGGGAGCTACATTCTCGATTTTGCTTGCTGCTCGGCGCGTGTAG
TCGTTGAGCTGGATGGTGGGCAGCATGATTTCAGTTGCCTATGATTCCAGGCGCACTAGCTGGCTTGAGTCGCAGGGCTGGACCGTGCTGCGTTTCTGGAATA
ACGAGATTGATTGTAATGAGGAGACGGNGCTGGAGATATTCTGCAGGAACTGACCGCCGGNCACCCTCTCCCTGAAGAGCGAGGGGCAGACCGAGCCGATAGC
GNTGTGGGAAAACATGAGACGGGCTGGAGATATCGCAGGGCTGACCGCCACCACGCCAGAAAGGTAGGGCGGTGAACGTTGTGT
>‘991108a-033.scf” came from CONTIG 19 at offset 0;
“E:\SEQUENCE\export\EST_db\991108a\991108a-033.scf’ (8>608)
GGCGTTAAATATGGATCCCCCGGCTGAGAATTCGCCGAGCGAAGCAAGAACCCTGTCTCCTAAGCTAAGCCAAGCAAAGCTTGAAGCCAAGAAGCAGGTTGAA
AGGTGTCCACAGCCACAAAAAAAAAGAAGATCCGGACGTCGCCCACCTTCCGGCGGCCCAAAACAGTGCGGCTCAGGAGGCAGCCCAAATACCCTCGGAAGAG
CGCGCCTAAGAGAAACAAAGTGACCACTATGCCATCATCAAATTCCCCCTCACCACCGAGTCAGCCATGAAGAAAATAGAAGACAACAACACACTGGTATTTA
TTGTGGACGNCAAGGCCAACAAGCACCAATTAACAGGCTGTGAAGAAACTCTTGACATTGACGTGGTTATGTCATACTTGATCAGCCTGATGGAGAGAAAAGA
TATGTTGACGGGCTCTTCTATGATGCTGGATGTTGNNCACAAATGCATATCTAACTGAGTCACTGCTATTNCAATTAAAGTTTACTTTAAAAAAAAAAAAAAC
TAAGGGGGCCGGACCCATTCGCCTTGGAGTGATACATTACTGNCGGTTTCAGCGGATGGGAAAACTGGTACCATTTCCCTGAC
>‘991108a-034.scf” came from CONTIG 20 at offset 0;
“E:\SEQUENCE\export\EST_db\991108a\991108a-034.scf’ (54>619)
GCACGAGGGTGCAGTCTCTGAGTGGAGGGAGGGCCGACCAGCGTATCTCCGTCTCCCTGGGGAAAGGTGCTGGTGAGGTGGACGACATGGCCTGGTGGAAAGC
TTGGGTTGAACAAGAGGGCGTCTCAGTGAAGGGCAGCCCCCACTTCAACCCACACCCTGACGCANAGACCCTCTACAAAGCCATGAAGGGGATTGGGACCAAC
GAGCAGGCCATCATCGACGTGGTCACGAAGAGAAGCAACGCACAGCGGCAGCAGATCGCCAAGTCCTTCAAGCTCAGTTTGGCAGGATCTCATCGAGACCTTG
AAAGCGAGCTGAGTGGGCAGTTGAGAGGGTCATCATATCCTCATGTACCCCCATACAGAACGAAGCCANGAGCTATATGATGCCATGAGGNCATAGAACCAAG
AGGGCATCATCNAATTCTGGCTCTCGGACCAGAACCACTCAGAGATATGAAGCATACAGAGACTATGGTCACCTGAAGAAACACAACAACACACGCTACTGAA
GATCGNGGCCTCTGCGCACAAAGACTGAGTATGGGACAGATGCCCGCAAGC
>‘991108a-035.scf” came from CONTIG 21 at offset 0;
“E:\SEQUENCE\export\EST_db\991108a\991108a-035.scf’ (48>532)
TTTTTACATGAAGCTACTCATATCAAGAGCAATAATCAGTCTTACAGCCAGACAGACTGTCAGTCCTCAAACTTGACTGTACTGACCTGACCATCTCCCTTTT
CTCTCCAGACAATTGATGATTCCCCTATTTTTAATCGAGGAGTGTCTGCTTTTCAACTACCCCTCCATGTCATACACCCAACCAATTTACAATAGTTTTATAT
CTAGGTTATTGCTTTTTTGATAGCACATCCATGTGTATTATTACTTTCTTTTATTTCTTAACTATCTGAATGGAGTATTTCGTGGAAAGCTACAGCTCTGTTC
ACTTTTTTGCTTCTACACTTTGCAGATGCATGGTATTANGCACTGCCTCTTTTTACATCTCTGATCATAATGCAGAGATTTTTCTTGTACACAAAGCAGATTA
TGATTCCTTCCACGAGGACACTTACTAGATGCTTGCTCGCCCGCGCCTATGTAGGATAGATCCATC
>‘991108a-036.scf” came from CONTIG 22 at offset 0;
“E:\SEQUENCE\export\EST_db\991108a\991108a-036.scf’ (48>649)
CGTCTGAAGATTTTCTATTGCTGTCAAGTGCCACCTCAAGGGCCATTCAGCGCCAACTTGCAGGTTTACTCTTTGAGTTGGGATGTACCAATTCAGCCCTTCA
GATATTTGAGAAGCTAGAAATGTGGGAAGATGTTGTCATTTGCTATGAAAGAGCTGNGCAGCATGGGAAGGCAGAAGAAATCCTGAGGCAAGAGCTGGAGAAA
AAGGAAACGCCAAGTCTATACTGCTTGCTCGGAGATGTCCTTCGAGACCACTCTTACTATGACCAGGCCTGGGAGTTGTCTCGACCCGCAGTGCTCGAGCCCA
GCGCTCCAAAGGCCTCCTCCACCTGCGAAAGCAGGAGTTCANAGAATGCGTGGAGTGCTTCGAACGCTCTCTGAAGATTAAATGGATGCAGCTCGGAGTGTGG
TTTTCTCTGGGCTGTGCCTATCTGGCCTGNAAGACTACGNAGNTCAGCGAGGCATTCAGCGGGNGTGACTCTGGAACCGACATGCTGAGCTTGAAACATTATC
ACTCTATATCGCTTAAACAAAGNAAAGCTTTAAACATACAGAGCCTCAGGCACTTGACATGCGATCGGAAACTACTTCCTACCTG
>‘991108a-037.scf” came from CONTIG 23 at offset 0;
“E:\SEQUENCE\export\EST_db\991108a\991108a-037.scf’ (48>672)
GGAGGCTCCATCCAGACCAGTGTGAATGCGCTGTCGTCAGATGTTCTGGGCCGCTGCCAGGTCTTTGAAGAGACTCAGATTGGAGGCGAGAGGTACAATTTCT
TCACTGGCTGCCCTAAGGCCAAGACATGCACTATCATCCTCCGTGGTGGTGCAGAGCAATTTATGGAGGAGACAGAGCGGTCCCTGCACGACGCCATGATGAT
TGTCAGGAGGGCCATCAAGAATGATTCAGTGGTGGCTGGTGGTGGCGCCATCGAGATGGAGCTCTCTAAGTACCTTCGAGATTACTCAAGAACCATTCCAGGA
AAACAGCAGCTACTGATTGGGGCCTATGCCAGGCCCTTGGAAATTATCCCACGCCAGCTCTGTGACAATGCTGGTTTTGATGCCACAAACATCCTCAACAGCT
ACGGGCTCGGCCGCCCAGGGGGCATGGGGTACGGGTGGACATCAACACTGAGGACATGCTGACAACTTGAGCCTTTGGTGGGACCACGAGTGCGGACAACCCC
TGCTGCCCTCGAGCCGGGCCTCATGGTTGTGATGAACATATACCCCCTANAGGATGCTCCCACCCTGNCGGGCCAGCGGCCCCTCCGAGTTCACCCCCACAGG
CCGCGTT
>‘991108a-039.scf” came from CONTIG 24 at offset 0;
“E:\SEQUENCE\export\EST_db\991108a\991108a-039.scf’ (47>609)
CAGACCTGAATTCCTTACAATCTGAGAAGTAAATAGATAGCACTTTGAAGGGGCGCCCAACAGAGTTGGAGGCGTCATCAAAACTTTGGGTTCCCCTTTGCCC
CCTCTAAGGGTTGGGCAGGGTGACCCTGAAGTGGGCACAGCCTGGAAATGGGGCTGGGGGATTGGACACCCTCCTGGCCCTTGATTCCCACCTCTGTCCTGTG
AAGGCATGGGGAAGGTAGGGGCCCGGTACAAAGCACCCCTGCCTCCCCACTCCCCCCTGACCCCCATCCTCAGGGAGCCAGTCTCAATGGTTGGCTTCCCTGT
GCCCTGCATTTTTTCATGAACCCCACAAGCCCCTGTTTCCCAACCAGCTCTCAATCCCTCTCTGCTCCTCCCCCTTCCTCCTGCCCTTTCCCCTTATATTTTC
TCACCGTGGTGGCAGCTTGGGGGGGGCCCTACCCCCACTCTATGACTGGAGGGGAAGACATGCTAGCGAGGGTCCCACTCTACTCAGCACGACGCCCCCTCTC
TACTCTGGGGGGGGCTGGGGNCGGCAGGCTCCTGATAGATTGGGTGGG
>‘991108a-041.scf” came from CONTIG 25 at offset 0;
“E:\SEQUENCE\export\EST_db\991108a\991108a-041.scf’ (54>671)
GCACGAGGGGCCGCTCTTCCTGGCTTTGGGGTTCTTTCTCCGCGTAACGTGTGGTCTCTGATATTCAACCCTCCTCCCAGCATAGGCTGCATGAGGCGGNGGC
GGGCTCGCACATTCCTGCTCGGCGGAGGGGTGAGTGACCGGCCCCGCCCCTTCCGGTCCTCGAAGCTTTGCCCATGACCCGTACCCCAAAACCCAGATGTTGG
CCTCCGAGGAGAGAGTGAAAACTGAGTATTTTGCCTTAAATTTTCCAAGCTTCTCTTGGTTTCAGGAATATTTTCCTTCGGGATGCAGAAGAGGAGGGCCTGA
GTGTTTATGCAAGCAAGTGTTCCGGGGTGCAGTTTTGCTGGGGTGTTTATACGACCCCGAATCCACCCCCTGCCCTTAGCCGCTCTGCTGCTGTCGCTGTTTT
GGATGCCGACACTGCTGCCTGCAGCCTCTCGCCTTCTGTTGCTACCCCGAGCCCTGCTGTCCATGCTCAGAAGTCCCCCGGCCACCCTCGCCGCCTCGGCTCC
GCTATGCCCGCTCGTCTCGCGCCTTGNCACTGCCCACCAGAGGCGCCAGAAACGCAGGGGGACTTAGTGAGGAGAAGGCTTAGAAGAGATGGGACGCCCCCT
>‘991108a-042.scf” came from CONTIG 26 at offset 0;
“E:\SEQUENCE\export\EST_db\991108a\991108a-042.scf’ (48>434)
CCGGTTCTCCCCCAACCCCGCTCCGCAAGGGAGCCGTGAGGGCCGCAGACCCTCACCTTTAGTGTCTCGGGAGCCAGACCAGTCCCCCTCACCCCACTATATA
ACAAACTCAGGGTCAGGTACTTCCCGCTTTTCCTTTACAGCAACCCTAAATTCAGGGGATTATGTTAACAGTACAGGGGATGTAACCAATAACCTATGGCCTT
ACCAATAGACGCTCCCCTGAACCAATCCGTCCCGAGGTACCCCAGCGCGGGGGGATTTCCGCCCGGCAGCGCCCCCAGACTCCTGCACCAACAGCCCGTCCAC
CCCACGGGTCACTGAAACAAATGAACGCGCAAACTATGCACCCTTTCCAAGCTTAGGATCGGTTCGCCTCCCCGCCTT
>‘991108a-043.scf” came from CONTIG 27 at offset 0;
“E:\SEQUENCE\export\EST_db\991108a\991108a-043.scf’ (51>526)
AAACCAAACTCTATCCAAAGTCCCAGAAAAAGCTTCACACTCCATATGTCCTTTTGTTCTAATCTTGTCAGCCAGTACAAGTGACCAAGTTCCAGTTATTTAT
TTCCAAAATTCTTGGGAAAAAAGTGTAATTTGACCAAAAAAGGATATTTGTTTTTGCTATTACACCAAATACAGTTCAAATGCTTATTGTTCTATTTTTTTAC
CAATTTCAATTCAAAATGTCTGAATGGTGCTATAATATATCTTCAACACTCTTCCAATAACACTGCGTTACATTCTTTGAATCCTAGCCCATTTTCAGAGCAT
GACGGNGCTTACCATTAATAATTACCTTTCTTCTGAAACAGGCAAGCAAGAAATAGAAAAGACTTGCGTGTCAACTAACTCACCCGGCAGAACTAAAGAATTC
TTGAGGCCAAAAAGAAAATTGTACGAATGGCCAACTAAGTTCTAAAATTGTTTAAAAAA
>‘991108a-045.scf” came from CONTIG 28 at offset 0;
“E:\SEQUENCE\export\EST_db\991108a\991108a-045.scf’ (54>653)
GCACGAGGATACGGCGTTGTCTTGAATTCCCGTCGTAACTTAAAAAAGGGAAGCTTTCACAATGTCCGGAGCCCTTGATGTCCTGCAAATGAAGGAGGAGGAT
GTCCTCAAATTCCTTGCAGCAGGAACCCACTTATGTGGCACCAACGTGACTTCCAAATGGAACAGTACATCTACAAAAGGAAAAGTGATGGCATCTACATCAT
AAATCTGAAGAGGACGTGGGAGAAGCTTCTGTTGGCCGCTCGGGCCATTGTCGCCATTGAAAACCCGGCTGATGTCAGTGTCATATCCTCCAGGAATACTGGC
CAGCGAGCTGTGCTGGAGTTTGCTGCTGCCACTGGAGCCACTCCTATCGCTGGCCGCTTCACTCNCGGAACCTTCACTACCANATCCAAGCCGCATCAAGGAC
CAAGCTTTGNGGTCACCCGATCCCAGGCTGACCACACCCCTCACGAAGCTTTACGGAAACTGCCACCATGCCCTGGCACACGACTCTCTTGGCTCGTGGCATG
GCTCCGGCACACAGGACGCCTANGGGTGAGGGGGAGCTGCCGGAGCCTGGCGGGCACATCTCGAACACCGGGAGCTGCGACTTCTTT
>‘991108a-046.scf” came from CONTIG 29 at offset 0;
“E:\SEQUENCE\export\EST_db\991108a\991108a-046.scf’ (54>638)
GCACGAGGCGCAGACTGGTTGCGCGCCTCTTTTTCTTCGCTGCCTCCCAGTTCTAGATTCCGGCGCCATGGGTTTTGGAGACTTGAAAAGCCCCGCTGGCCTC
CAGGTGCTCAACGACTACTTGGCGGACAAGAGTTACATAGAGGGGTATGTGCCATCACAAGCAGATGTAGCAGTGTTTGAAGCCGTCTCGGGCCCAGCACCTG
CCGACTTGTGTCATGCCCTCGGTGGTATAATCATATCAAATCCTATGAGAAGGAAAAGGCCAGCCTGCCAGGAGTGAAGAAAGCTTGGGCAAGTATGGCCCTG
CTTATGGGGGAGACACCACAGAAAGTGGAGCTACAGATAGTAAAGATGATGACGACATTGATCTTTTGGATCTGATGATGAAGAGAAAGNGAAGAANCAAGAG
ATAAGAAAATACGCCTTGCCAGATGAGTAAAGAAAGGAACCACACTTGTCGCAAGTCTCCTCTTATTTACAGAAACTGGATGAGAGACGAATGCAAACTAAGA
GGGGCAGACATCAAGCAAGCTGGGCGGGCTCTTTACAATCGGTGGTGCATAAAAACAAACAG
>‘991108a-047.scf” came from CONTIG 30 at offset 0;
“E:\SEQUENCE\export\EST_db\991108a\991108a-047.scf’ (48>612)
CAGTGACCCAACTGCGGCAGATGCTGGAGAATGAGAAGAACTCCTCTGTGCCCCCTGGCTGAGCATTTGCAGGGTAAAGAAGCATTTGAGAAAAAAGTTGGGA
ATCATTAAAAGCTAGCTTGAGAGAAAAGGAAAGAGAAAGCCAAAACAAAACTGAAGAAGTCTTCCAACTCCCATCTGAGATTCAAAAAACTTAAACAAGCCGT
TAAAAAATTAAAGACTCGGGAGGTGGGTGATTTGTCGAAATATAAAGCAACGAAAAGCGNATTGGAGACACAGATTTTCGACTTTATACGAAAATTGGCCAAT
CTGATAGGAAGTATGAGGAAGTATGGAGGAGGGTTTTTCATGCCCAAAAAGAAGAACTGTCTGCTAAGATGAGAAGAATTGCTCCATTTCACATANAGCAAGA
AACAAAGATCAGCAGAACGATGTGACAATCCTTCACACCATCACGAGCTACAAAAAAATACAGATCTGCCAACAATCAAACAAANAATAAATACTGACGCTCA
GAGTGAGAGATAAACGGCTCATGCCTTCCACTCCCATGAAGGAGCCACAG
>‘991108a-049.scf” came from CONTIG 31 at offset 0;
“E:\SEQUENCE\export\EST_db\991108a\991108a-049.scf’ (54>370)
GCACGAGGGAAAGATCCTGCACATTGACTTTGGGGGACTGCTTTGAGGTTGCTATGACCCAGAGAGAAATTCCCAGAGAAAATTCCATTTAGNACTAACAANG
AATGCTGACCAATGCTATGGGAGGTCACGGGGCCTTGATGGCAACTACAGGATCACGTGCCACACCGCGATGGGAGGTGCTCCGGGAGCACAAGGACAGCGGC
ATGGGCGCGCTGGGAGCCTTCGTCTATGACCCCCTGCTGGACTGGAGGGTGGAGGGCACCAATACCAAAGGGAACAAGCGATCACGGACGAAGACAGAATCCT
ACTCTGCT
>‘991108a-050.scf” came from CONTIG 32 at offset 0;
“E:\SEQUENCE\export\EST_db\991108a\991108a-050.scf’ (48>566)
GTCGTGGGCTCACACCCGGCTTCTCTTCAGCCTTAACCTGTGCGCCGCCATCGCCGTCATGCTAGGCGCCGCTGTCCGCCGCTGCTCTGTAGCTGCAGCCGCA
GTCGCCCGGGCCAGCCCTCGAGGCCTCCTGCACCCCACTCCGGCCCCCGGCCAAGCCGCCGCTGTCCAGTCACTTCGCTGCTACTCCCATGGGTCACATGAGA
CAGACGAAGAGTTTGATGCTCGTGGGTGACATACTTCAATAAGCCAGATATTGATGCTGGGGATTGCGGTAAGGGATGAACACACTGNNTGCTTGANCTGGNT
CCAGAGCCAAAAATCATGATGCTGCTTGCGGCATGCGAACGTTTAATGATTTGTAGNGCAGTCGCATCTAAAAGTGGTAAGACAAAACAGAACTATAAGAATC
TACCCTTGTATTCAGAACTATACAACTTGATGACTGGAATTCACTCAAGAACGGCCTGAAAGGTAACCCAGATGACTCCAGACTATGAATGTATGATTAATAA
CGAA
>‘991108a-051.scf” came from CONTIG 33 at offset 0;
“E:\SEQUENCE\export\EST_db\991108a\991108a-051.scf’ (48>407)
CTGTTTTGAGTGCACCGTGAGTGATGTGTGTCATGCCTCTGGGGTGGAGGGAAGGGACCGACACAGACGTACACTCCCACACTCCACCTGGTGAAAAAGGATG
CCTGGACGAAGGTGGGTTCGGACCTTGGTCCTTGTGTGTGTAATGCCTTTGTGTCTGATACCAATGTATGGGCGGTTTCCAGCTGATATGGGTCCTGCCTCCC
CTACCTTCAACCCCCACTACACGCTGTACCCCAGTATGACCCCCTCTTTCCTAATGCCCATGGAATGTGGGATNTCGGGCACCCACACTGTCCGCACGCCTCT
TCTATTCGCACTGTTGCCTCTCCTCAACCATTAATCCTAACGGCCACATG
>‘991108a-052.scf” came from CONTIG 34 at offset 0;
“E:\SEQUENCE\export\EST_db\991108a\991108a-052.scf’ (54>747)
GCACGAGGGCTGACTATTCTCAACCAACCATAAAGATATTGGGTACCCTTTATCTACTATCTTGGCGCTTGGGCCCGGCATAGCAAGAAACAGGTTCTTTAGC
CCTTTGTAATTTCGGCGCTTGAATTAGGCCCACCGCGGAACCTCTGCTGGGGAGACGACCCAAATCTACAACGGAGGTTGGAACCGCACACGCATTTGTAATA
ATCTTTCTTCATAGTAATACCAATCATAATTGGAGGATTCGGTAACTGACTTGTTCCCCTTAATTAATTGTTGCTCCCGATATAGCATTTCCCCGAATAAATA
TTATAAGCTTCTGACTCCTCCCCTCCCTCATTCCTACTACTTCCTCGCCTCCTCTTTAGTTGAAGGTGGGGCAGGAAAGGCTGAACCGTGTTCCCCTCCTTAC
AGGCAACCCTTCCCAGCGGGAGCTTCTGTAAAATTTACCCTTTTTTCTTACCCTTTACAGGAGTTTCTCATTTTTAGGACCTCACTTCTTTCATCATTTTAAC
ATAAGCCCCCCGCAGTAACATACCAACCCCTTGTGGTTGTTCGTATATTCCGCGGTCTTTTCTTTTTGTTCTGTTTTCAGCGGTTCTAGTTCTTACGACGAAC
CAATACCCCTTCGCCGGGGAGGGAACCCTTTTTCTCCCTTTCTTCCTGGCACCCGACTTTTTATCTCCGGCCGGG
>‘991108a-081.scf” came from CONTIG 34 at offset 0;
“E:\SEQUENCE\export\EST_db\991108a\991108a-081.scf’ (47>736)
TGACTATTCTCAACCAACCATAAAGATATTGGTACCCTTTATCTACTATTTGGTGCTTGGGCCGATATAGTAGGAACAGCTCTAAGCCTTCTAATTCGCGCTG
AATTAGGCCAACCCGGAACTCTGCTCGGAGACGACCAAATCTACAACGTAGTTGTAACCGCACACGCATTTGTAATAATCTTCTTCATAGTAATACCAATCAT
AATTGGAGGATTCGGTAACTGACTTGTTCCCCTAATAATTGGTGCTCCCGATATAGCATTTCCCCGAATAAATAATATAAGCTTCTGACTCCTCCCTCCCTCA
TTCCTTACTACTCCTCGCATCCTCTATAGTTGAAGCTGGGGCAGGAACAGGCTGAACCGTGTACCCTCCCTTATCAGCCAACCTAGCCATGCAGGAGCTTTAG
AGATCTAACCATTTTTCTTTAACTTAGCAGTAGTTTCCTCATTTTTAGGAGCCATCAACTTATTTAAAAAATTTTAAATTACAGCCCCCGCAAGCCACAATAC
CACACCCTTCGGTTGTTGAATCCGTATAATTACGGCGTCCTCTAATATTTCGCTTCTGTTTTACAGTCGCTTCACAGTCTTTAACGACCGAACTTAATCAACC
TTTTGCCCGGCGTGGGGGAACCTTTTTTTTTACCTTATTTGTTTTGGCCCCCGTATCTTTTTATTTAACGGT
>‘991108a-054.scf” came from CONTIG 35 at offset 0;
“E:\SEQUENCE\export\EST_db\991108a\991108a-054.scf’ (47>642)
TCAGGACAGCTACTGTAACTCCATCCTGAGGCTCATGTACGCATCTTTTCCTGAGACTCAAAACAACAAAACAGGGACTTAGATTTGGAAACTGACTTGGTCT
GATGAACCAGACCTGTCTGAGCCTGCATGCCAGGAGCTGATCAGCTCCTCTGTACTTGCAAATCTAAGTTTTGCTCTTTCCTTATAAGATGTGTGATCCCTGC
AGTCTCTAATCCCCAGATGGAGGCCCACTCAGGTTATCATCATCTTCAGATGGGGCTTCCCAGGTGACGCTAGTGGTAAAGAACCAGCCTGCCAATGCAGGAG
ACGTAAGAGACATGAGTTCAATCCCTGGCTGGANAAGATCCCCTGGAGAAGGAAATGGNCACCCATTCCAGTATTCTTCCTGGAAAATCCATGGACATANGAG
CCTGGGGGCGCAGATGGGAGATGACGNAGTGACTAACATAGCAGNCAGAATGGCAGGCATATTACTATACACAAANGCTCAATGGNGAACACTGTCTGAACTT
CTCTGCTTGTTATACACAGAATCATATATACCACACACCCATTTACGAGAAAAGTGATTTATCCAGACCCGACACCGCTC
>‘991108a-057.scf” came from CONTIG 36 at offset 0;
“E:\SEQUENCE\export\EST_db\991108a\991108a-057.scf’ (53>131)
GCACGAGGAGCCGCTTCATTCTGCCCATCGGTGCCACGGTCAACATGGACGGTGCCGCCCTCTTCCAGTGTGTGGCTGC
>‘991108a-060.scf” came from CONTIG 37 at offset 0;
“E:\SEQUENCE\export\EST_db\991108a\991108a-060.scf’ (194>353)
ATGGCTTTGTGGGGATTCCTACTTTTCCTGACTAGGGTATTGGATTCCATACCCCCTGGCAGTGAAAAGCCTAGAGTCCTAACCACTGGACCACCAGGGAATT
GCCTATTTTTTTAGGGCTGAAAACCTAAATAGATGATGAATATACTTAAGCAGGAG
>‘991108a-061.scf” came from CONTIG 38 at offset 0;
“E:\SEQUENCE\export\EST_db\991108a\991108a-061.scf’ (47>637)
TGGAGACTGGTGTTCTCAAACCTGGCATGGTGGTCACCTTTGCTCCAGTCAATGTAACAACTGAAGTGAAGTCTGTAAAATGCACCATGAAGCATTGATGAAG
CCCTTCCTGGGACATGTGGGCTTTAATGTCAAAAACGTGTCTGTCAAAGATGTCCGTCGTGGCAATGTGGCGTGGTGACAGCAAAAATGATCCACCCATGGAA
GCTGCTGGCTTCACAGCTCAAGTGATTATTTTGAACCATCCANGCCAAATCAGTGCTGGATATGCACCTGTGCTGGATTGTCACACAGCTCACATTGCTGCCA
GATTGCTGAACTGAAGGAGAGATTGATCGNCGGTTCTGGAAAAAACTGGAAGATGACCCTAATNCTTGAAATCTGGGGACGCTGCATCGNTGTATGGGTCCTG
GCAGCCATGGNGNGAGAGCTCTCTGATTATCCTCCCTGGCCGCTTGCTGGCGNGACAGAGAAGACAGCGCTGGGGGGATCCAGCANGGACAAGCAGCTGAGCT
GCAGGCACAGNCGCCCAAAGCCAAAGATAAAGAAATATCCCATACTGCCCCCCCTATAGGGGAAGACGGCCAACT
>‘991108a-063.scf” came from CONTIG 39 at offset 0;
“E:\SEQUENCE\export\EST_db\991108a\991108a-063.scf’ (47>551)
CTTGGTTGTGTCTGGAAATCACCCCCGTGCAGCTGTCCTTCATGTGCAGGAGTGTTGCCCCAGTGCTGGTGCAGAGGTGACTGTTTCCTGGCAGGGTGTGCCC
AGGGCTTCCACAGAGGAGCCTTTTTTCCTTCCCCAGAAATGTCTCAGTGAAGAAGGGGGCAGGACAGGTGGCCTTGGGGACAGAAAGCAGACTAGACTTGCTG
TCATAGTGGAAGCTGGCCCTGTCCTCAGCACATAAGCAGCATTGGGAGCAGTCAAGCCTGGCATCCCTGAATTGGGGTTCAGGGGGTGTGCCCAGGTGCCCTC
ATGTCCCCACCCCCTCAGACTGCTGCACCCCAGACTGGGATGCTAGCCAGGGACACAGACCTACATGTGTGTGTGCCCTGGTGTGCATGTGTGCCTGTGGGGC
TGTGGGGATGGTCAAGCTCTGCCTGCCCTCTGTCTGAGCCATCTTGTGGACTTGGATTTTGCGCTTAGGAGGTGGTCTGGTGATGGGGGGGAG
>‘991108a-064.scf” came from CONTIG 40 at offset 0;
“E:\SEQUENCE\export\EST_db\991108a\991108a-064.scf’ (47>445)
CGCTGGGGAAAGGGCCTTTAATCGCGAGAGTTCCGTTCTCTTGAGAGCGCCTCCGCCGGCGCCTAGATCGCGCGAGACCGCGGAAGGAACCGAAGCGTGTGGC
GCGCGCGGCGGCCGCGACGGGAACAAGATGGCGACGGCGACCATAGCGCTACAGGTCAATGGCCAGCAAGGAGGGGGGTCCGAGCCGGCAGCAGCGGCGGCGG
CGGCGGCAGTGGTGGCAGCGGGAGACAAATGGAAACCTCCACAGGGGACAGACTCCATCAAGATGGAGAACGGGCAGGGCACAGCCGCGAAGCTGGGACTGCC
TCCCTTGACGCCCGAGCAGCAGGAGGCCCTCCATAAGGCCAAGAGTACGCCATGGAGCAGACCGAGCGGCTGTGAAACATACCTCG
>‘991108a-065.scf” came from CONTIG 41 at offset 0;
“E:\SEQUENCE\export\EST_db\991108a\991108a-065.scf’ (47>511)
CGCCCGCACAAGTGAAGAAGCACTACCGCCGTGCCGTGCTGGTGGTCCACCCCGACAAGGCCCGGGGGCAGCCCTACGAGCAGTATGCCCGGATGATCTTCAT
GGAGCTCAACGATGCGTGGGCTGAGTTTGAGAGCCAGGGCTCCCGGCCACTCTTCTGACCTGCAGGCACGGCTGCGTGTCTGGCTCTGGAGCCGGTGGTGTGA
GCGGCCCTCGAGGGTGGCCAGGGCTCCGGCGGCGTGGGCAGGCGTGGCTGCACCCGGTGGTCGCGTCGTGCCCATGTGCTCAGCGGGTCGGAGCCGATGGCGC
TCCCGGCAGGAGAAGAGAAAAGCATTCCAAAGCCTCCCAGTCTCTCTTTTCTGTCTTGGCCCCAAGAAACGTGCACTCCGTGCTCCACGCGTGTACGCTTGAT
CGTTNTGTCCAGGGCGCGTCACGAGTGGGCGCGCTGGCTGCTACTGCAGAGCGT
>‘991108a-066.scf” came from CONTIG 42 at offset 0;
“E:\SEQUENCE\export\EST_db\991108a\991108a-066.scf’ (47>571)
TCGAGTTTTTTTTTTTTTTTTTTTTTTAACTGTATAAACTATTTATTAACACAAAGCCCACACATTATTTCTTCTTGGACACACCCACAGTGCGACCACGGCG
GGCCTGTGGTCTTGGTGTGCTGGCCTCGGACACGGAGTCCCCAGAAGTGGCGCAGCCCCCTGTGGGCCCGAATCTTCTTCAGGCGCTCCAGGTCTTCACGGAG
TTTGTTGTCTAGACCGTTGGCCAGGACCTGGCTGTATTTCCCGTCCTTCACGTCCTTCTGTCTGTTTAGAAACCAGCTGGGATCTTTGATGGGCGGGGATTCT
GCATAAGGGGGATCACACGNTCCACCTCTCCTCGGTGAGCTCCCCGCCCTCTTGGGAGGGCGATGTCTGCTTTCCTCACACCACAGAGCATATCTTCCCCCCA
CCCTAATGCAGAGAGGGAAAGCAATTTCCGCCGCCATGATTGTTGTGAGACTCCAGAGTGCTGGACTCTAGGATTCTAAAAATGGGGGGCCTACGGGGGGGGG
CGGACCAACC
>‘991108a-067.scf” came from CONTIG 43 at offset 0;
“E:\SEQUENCE\export\EST_db\991108a\991108a-067.scf’ (53>641)
GCACGAGGTGTTGCGCCACTTGCGCCCTGGGCAAGGGATGCCCTGTGGGGTGTACACCCCCCGCTGCGGCTCCGGCCTGCGCTGCTACCCGCCCGGGGCGTGG
AGAAGCCCCTGCACACGCTGGTGCACGGACAAGGCGTGTGCATGGAGCTGGCAGAGATCGAGGCCATCCAGGAAAGCCTGCAGCCCTCTGACAAGGACGAGGG
CGACCACCCCAACAACAGCTTCAGCCCCTGCAGCGCCCACGACCGCAAGTGCCTGCAGAAGCACTTGGCCAAAATTCGAGACCGGAGCACCAGTGGGGGCAAG
ATGAAGGTCATCGGNGCGCCCCGAGAGGAAGCCCGGCCTGTGCCCCAGGGCTCCTGCCAGAGTGAGCTGCACCGGGCGCTGGAGCGGCTGGCCGCCTCACAGA
GCCGCACCCACGAAGAACCTTACATCATTCCCATCCCCACTGCGACCGCAACGCAACTTCCACCCCAGCAGGCCCACCGCCCTGGATGGCAGNGCGGCAGGCT
GGGGGNGGGACGGAGACGGAGGGAAGCTTCGGGGGCTGGAGCGAAGGGGAGCGAACTGCACAGTGCTGACACTT
>‘991108a-069.scf” came from CONTIG 44 at offset 0;
“E:\SEQUENCE\export\EST_db\991108a\991108a-069.scf’ (47>434)
CTCCCATGCTCCCTCTTTTGGCAAACTGTAAGTACTGTCTAGCAAACAGCGATAATTTCACAATTTCCTCTCTAGAATTGCTGAGAGACTTGAGTCACCAAAC
GTGGTTCACTACTGTGGAAATGCATTGTTCAGAACACAGAAAGAAATTTAAGATTTCTGCTTTTCTGTGACAGTATTTGTTTCCGCCTCAGTTCAGAGTGTGA
ACTGCAGGTTCCTGGCAAGACTGCTATTTAACATTGCATGTGAGAACCTGGTAAATAGGCAGAAAATCTTGGAGCTTGAAAGGAACCTCAGAATGATTCATAT
GGTCATTCATTAGATTGGGACCTTTAAGGGATCTCTTTCAGCCTCTCTCAGTGCAGAAATCAGGACAGGGGTGCTTGG
>‘991108a-070.scf” came from CONTIG 45 at offset 0;
“E:\SEQUENCE\export\EST_db\991108a\991108a-070.scf’ (53>606)
GCACGAGGGTTGGTCCTGTTTGTTTTCTTGGCTAAGATTAGGACAAAGAACATATGAAATCAACAGAAAATATACCTTGGTACCACCAACCCATTTTATGCCA
CATGCAAGTTTTGAATAAGAATGGTATAGAAAATAACTTGCTACATATGTATGTACCAATTAGGAAATACTGATGCCCTTGTGGGCACAGAACCACATGACAA
AACTTTGAAAATCATAAAAATATAAAGATAGTGTGGCTGAGATGGAAACAGGCCTTATTCTTGAATCCCAATTTTCATCTCTCCTTTTCCTATTTGGATTTCT
TTGGTGCTGTAGGAAAAAAAAAAAAAGAGAGAAAAATATATATTCATAAAAGATATGGNGCTCATTCCCATCCATCAAGGATGTGCTAAAACAATGTGTTTAA
TAAATTGTAATTTTATGTACAGGTCTATACTGTTATCTATGTGTCCATTTTCAAAACTGCACGTGTCTCTGAATTCATCTGACTCTATTTTTGACATTGCAGA
TGATGGCATATATGTATATGAAAAAATAAACGTT
>‘991108a-072.scf” came from CONTIG 46 at offset 0;
“E:\SEQUENCE\export\EST_db\991108a\991108a-072.scf’ (53>612)
GCACGAGGCTGGAGCATAAGAACAGTGCTTATTTTCTTCTTTTCCAAGAGAACAGAAAGAAATAATGAAGCATTGGATGATTTAAAATCCCAGAAGAAAAAAA
TACTTGAAGAAGTCATGGAAAAAGAAACTTATAAAACAGCTAAATTAATCTPGAAAGGTTTGATCCAGATTCGGCAAAGGAGTTTGAGCCACCGTCTGCTGGA
GCAACTGTAACTCCCAGACCTGGACAAGAATTCGTCAGCGAACTGCCGCTCAAGAAACCTTTCTCCACACCAGCAGGCTGCAGCCAGGCCCTCCTCTGCCCAG
TTTCTCCTGGACACCAAGGACACTTCAGCCCCTGGTGGACCCCCAGAAGACTGNTACTCAGCCTATATCAATGTGGTACAAGACGTNTGGACCCCTGCTCTCA
GNGCTGGATGGGNCTCTCCTCTGGGCACCTTACAGACCATCTGCTCGAGACAGNGCTCTGATAATGTGATATTATGNGAGGCCCAAAAGNACCCCCTATGCNA
CAGTTTTTACATGCTGTTTGAAGAAATGAACATG
>‘991108a-074.scf” came from CONTIG 47 at offset 0;
“E:\SEQUENCE\export\EST_db\991108a\991108a-074.scf’ (47>565)
CAAATTGAGATTTGCCAATGGAAGCATAAGAACATCGGAACTGCGACTCAACATGCAGAAGTCGATGCAGAGCCATGCCGCGGTGTTCCGTGTGGGGAGTGTG
CTGCAGGAAGGCTGTGAGAAGATCAGCAGCCTCTACGGAGACCTGCGGCATCTGAAGACGTTCGACAGGGGAATGGTCTGGAACACTGACCTGGTGGAGACCC
TGGAGCTGCAGAACCTGATGCTTTGTGCTCTGCAGACCATTCTACGAGCGGGAGCCCGGAAGGGAGTCGCGCGGCGCCCACGCCAGGAGGACTTTCAGAGAGG
TTGACGAGTACGATTACTCCAGCCCATCCAGGGNCAGCAGAAGAACCCTTTGAGCACACTGGAGGAGCACACCTCTCTACGTTGACATCAAGATGGAAAGNGA
CCTGGAGTACAGACTGTATCGAAGAACTTGACGAGATGACTGCGCACTTCCCCACCTNCGCTCTATGAGAGATGAGCTTCCTGTCTGTATTGATATACTCAAG
CGG
>‘991108a-079.scf” came from CONTIG 48 at offset 0;
“E:\SEQUENCE\export\EST_db\991108a\991108a-079.scf’ (53>531)
GCACGAGGCCAGATACCGCCCCCGGGCCCCCATCATTGCTGTGACTCGGAATCACCAGACAGCTCGCCAGGCCCACCTATACCGCGGCATCTTCCCTGTGGTG
TGTAAGGACCCAGTGCAGGAGGCCTGGGCTGAGGACGTGGATCTCCGGGTGAACTTGGCCATGAATGTTGGAAAGGCCCGAGGCTTCTTCAAGAAGGGAGACG
TGGTCATTGTGCTGACCGGGGGGCGCCCTGGCTCCGGCTTCACCAACACCATGCGTGTAGTTCCTGTGCCATGATGGACTCCGAAGCCCCTCCTCCAGCCCCT
GTCCCACCCCTCTTCCCCAACATCCGTTAGGCCAGCATTGCTTGTAGTGCTCACTTGGGGCTGTAATGGGGCCTGTGGGGCGGGACACCAGGAAAAAAGAGAA
AACCTTCTGGAAACCGGCGGTTTTAAACTTTGCTGGGCCGGGTAGCTACACCTGGGCCCTCTCACG
>‘991108a-080.scf” came from CONTIG 49 at offset 0;
E:\SEQUENCE\export\EST_db\991108a\991108a-080.scf’ (53>448)
GCACGAGGGGAAGCGAGGCCGGAGCTGCGGGCGCTTTTTCTGCCCGCGGTGTCTCAGATTCATTCTTAAGGAACTGAGAACTTAATCTTCCAAAATGTGCAAA
AAGACCATCTTATGCCCCACCTCCCACCCCAGCTCCTGCAACACAAATGCCCAGCACACCAAGGTTTGTGGGATACAATCCATACAGTCATCTCGCCTACAAC
AACTACAGGCTGGGAGGGAACCCGGGCACCAACAGCCNGGTCACGGCGTCCTCTGTTATCACGATTTCAAAAACCCCCAAGCCACCAGATAAGCCGCTGATGC
CCTACATGAGTACAGCAGAAAGGNCTGGACCAAATAAAGGCTTCCACCCTGACTANAGTGTGGGAAATGGGGAGATATTGGGGGNAT
>‘991108a-082.scf” came from CONTIG 50 at offset 0;
E:\SEQUENCE\export\EST_db\991108a\991108a-082.scf’ (53>377)
GCACGAGGGTTTGCACTCAGGTCCACGTGGCTCTGAGAAGGGTTCAGGCATCCGAGCTGTGGGGAGCCCTGGGTGAAGCTGGTTGTCCAGCGGYFCAGCCTCC
TTGGAGGAAGCCCTCTGCCCGCCCACTCCAGAGCGTGAGAGTGTGGGGGCGTGCCCAGGGAAGGTGCCTGGGAGGACGCAGTGACCACGCTCCCCTGGCACCA
CGCAGAAGTGGACGAGAAGCCCAGCCCACACCCCANGCCTCCTGGCTGTGCCCTGCGGCTGCTGCATTTGCTTTTAATTCTCAACACTGGAAAATACTGACTT
TGGGTTGGGCGGGGCC
>‘991108a-083.scf” came from CONTIG 51 at offset 0;
“E:\SEQUENCE\export\EST_db\991108a\991108a-083.scf’ (21>581)
TCTAGGTGGATCCCCCGGTGGTGGGGGTTGTGATGTGACATCTGTTTTTTATACCCTGTCGTGGGCATTCGCGGTGGACGCCACACGCCCGCTGATGGCCGAC
ATGAGGATAGACCAGGGCCGTAACCCTGCCTAAGTCCACACCTCTACCTTGCGTTTTATGTAGTTCTTTGCATTCTTTGTGGTGGTTGATGTTATTCCATTTA
TTGTGCTTTCATTTGGTTGGGGATGCCTGATCATTCCGGGGGGGGCTTTCCATACGCAGGATTGGCCTGCTTGAATTCCCGTCATTGGTTGCCCCTTCCCGCC
GGGCGTAATGGCCTGGCGGGGGGGCGTGTATGTGGCCAAATGATACACCGACCGAATGGCCGCCACGGGCTCATGTCCGCAGGAGGCCTTGTGGTGATATATC
TAACCCCCCGGGCTCTCTGGGATGAAAACCTATTTTGCCCTTCAACACTCCCGGGGGGCTAAATGTTTGGGCCTTTCGGTGTTCTCACCCACAGGGATGGCTA
CCCTTCACTGGTCAATATTCATTGTGTACACTGCCTCGTTTGCGC
>‘991108a-084.scf” came from CONTIG 52 at offset 0;
“E:\SEQUENCE\export\EST_db\991108a\991108a-084.scf’ (53>540)
GCACGAGGGTTTGGCCCAGACCCAACCATGATCCGAGCCAACTGCCTGGTCCAGACCACAGAGTGGAGTGCCTGTTCCAAGACCTGCGGAATGGGCATCTCCA
CCCGGGTTACCAATGANCACGCATTCTGCAGGCTGGAGAAGCAGAGCCGCCTCTGCATGGTCAGGCCTTGCGAAGCTGACCTGGAGGAGAACATTAAGAAAGC
AAAAAGTGCATCCGGACCCCCAAAATCTCCAAGCCTATTCAGTTTGAGCTTTCTGGCTGCACCAGCATGAAGACATACCGAGCTAAATTTCTCGGGAGGTGGC
ACAGACGGCGGGGCTGCACCCCCCACAGAACCACCACCTTNCCGTGGAGTCAAGTGTCCTGTGNNGAGGTCATGAAGAAGAGATGATGTTCATCAGACCTGTG
CCTGCCTTACACTGCCCCGGGACATGACATCTCGGTCCTGTTCTACAGAGATGTATGAGACATGGCTAAGCCGAACA
>‘991108a-085.scf” came from CONTIG 53 at offset 0;
“E:\SEQUENCE\export\EST_db\991108a\991108a-085.scf’ (47>641)
CCGCCCGCTGCTCCGGCAGCCCCCACTCTGGACTCTCCCTCCTGGCTTCAGCCTTACCGGAGCCAGGGNGATCCTGCCCGGAAAAGGGAGCTCTGATGTCCCC
TAGGAGAGGGAAGCTGGAGCCCACCCCCAGACAGCTCCTCCTCCCTTTCTGGCTGGCCCACCTGGCCGGCCCTGAGGCCTCACTGTAACCACAAGACCTTGTC
TCTTCGCCTTATTCTTCTCACTGCCTCCTTGGGTCCCTGGCTCCTGCGAGGCTCTGTGGAGCTGGCTCCAGCAGCCACTTCCCTGCTTCCTCCTGCCTCTCTC
CTGGGCAACTGGAGATGCCAGAATCACTGCCGGCTGGGCTGGTAGCCGGGGCTGGCCCTCCCCCTTCTGCCTGCTGGGGAAAGAAAGGCTAAGCTGGGTGGAC
TGGCCGTGCTGAGGTCCTCCCTGTCTGGGTGCAGGGAGCACCCAGATGACAGCACCCTGTTCTGTCATCTCCCTTCTACGCCTTCACCTTGGCACTTCCCAGT
GAAACGACAGCAGCTGGAGTCCACACGGCCTTTTTTCCGCCATCTTGGTCCGCCTTCCCNATGTACTGCCCCCCAGCGCCCT
>‘991108a-086.scf” came from CONTIG 54 at offset 0;
“E:\SEQUENCE\export\EST_db\991108a\991108a-086.scf’ (48>535)
GTGAGAAAGGAGCCCGTGGTGCTGACGGACCTGGTGGAGCTCCTGGCACTCCTGGACCTCAAGGTATTGCTGGACAGCGTGGTGTGGTCGGCCTGCCTGGTCA
GAGAGGAGAAAGAGGCTTCCCTGGTCTTCCTGGCCCCTCTGGTGAACCCGGCAAACAAGGTCCTTCTGGAGCAAGTGGTGAACGTGGCCCCCCTGGTCCCATG
GGCCCCCCTGGATTGGCTGGACCCCCTGGCGAGTCTGGACGTGAGGGAGCTCCTGGTGCTGAAGGATCCCCTGGACGAGATGGTTCTCCTGGCGCCAAGGGTG
ACCGTGGTGAGACCGGCCCTGCTGGAGCTCCTGGTGCTCCTGGCGCTCCCGGTGCCCCCGCCCTGTCGGACCTGCCGGCAGACGGNGATCGTGGTGAGACCGG
TCTGCTGGTCTGCTGTCCCATGGCCGCGTGTGCCCGGGGCCCGCTGACCCCAGCCCCGGGGGACAGGGAGACAGCA
>‘991108a-087.scf” came from CONTIG 55 at offset 0;
“E:\SEQUENCE\export\EST_db\991108a\991108a-087.scf’ (48>579)
CGAGTACACACGGGCTCCACATCCTCAGAGTCAGCCAGCCGCGGGTCCGGAGGGCCAGTTGTACTGCCTTTTTATATAAGGAACTTGGGCATTCGCGGATGTA
GGCGGNGGGATGGAGTCCTGCAACCAGTCCCCCACGGATACCGGNGGTGACTGTGTATTATATTTGACCTAAATCTTTAGTGGGTAACAATTTTATGCAGTTT
GAATGAATAAAAATATTTTCCTGTTGTTTAGTTTTATTTGTATGTATTTTACTTTGATGAATGATTGGTTCCGAGGCCTCTGCCACACTCCAGAAATACTTGT
GTGGGCTGTTTAAAAAAAAAAAAGCTGTGTGTCTGTTCACTTATTTCTCTAAATTATCTCATTGCCTGGCATCAATCTTTCTCATATAGTTGTCCTAACCATT
ATGTACACANAAATGAAACAGATGGGAAGGAGACCAGAAAAAAAANTGATAATAAGAAAATGATGTTTGGCTCCATGTTTACATTTTTTTATAAAAAGTGCAG
AAGGAAAAAAACTGATAC
>‘991108a-089.scf” came from CONTIG 56 at offset 0;
“E:\SEQUENCE\export\EST_db\991108a\991108a-089.scf’ (55>589)
GCACGAGGCCCTGGCTCTGCTGGTTCTCCCGGCAAAGATGGATCTCAATGGTCTGCCAGGCCCCATCGGTCCCCCTGGGCCTCGAGGGTCGCACTGGTGGATG
CTGGTCCTGCTGGTCCTCCCGGCCCTCCTGGACCCCCTGGTCCCCCNAGTCCTCCCAGCGGCGGCTACGACTTGAGCTTCCTGCCCCACCACCTCAAAGAAGG
CTCACGATGGTGGCCGCTACTACCGGGCTGATGATGCCAATGGGTCCCGTGACCGTGACCTCGGAGTGGACACCACCCTCAAGAGCTGAGCCAGCAGATACGA
GACATCCGNAGCCCTGAAGCAGCCGCAAGAACCCGCCCGCACTGCCGTGACTCAGAAGGCCACTCTGATGGAAGAGCGAGAATATGGATTGACCCACCAAGGT
GCACCGGATGCATAAGTCTCTGCACATGGAACGNGGACTGGTTACCCACTCACCACGGGCCAAAAATGGATATACAGACCCAAGAAAAGACGCGGACGGGAAG
AGACGCGATCCATGAGTGGG
>‘991108a-092.scf” came from CONTIG 57 at offset 0;
“E:\SEQUENCE\export\EST_db\991108a\991108a-092.scf’ (56>279)
CACGAGGCAGGGCTCTATACCCCTCACATTGCCTGCTTACCTTCATACTTGCCCGAAGCTTGTCCTGCTGCCTGCCTGTCCTGCACACTGAGGAGTTCTGCAG
ACACACATGAGGGTCCTGAGATACTTCATTTCTCCCTTTGCGCTGACACGATAGGGCGCTTGATCTGATCCATCTACTGGTATTACCTTGTGCTGATGATGAG
AGATGCGTCGGGAAGAA
>‘991108a-094.scf” came from CONTIG 58 at offset 0;
“E:\SEQUENCE\export\EST_db\991108a\991108a-094.scf’ (50>650)
CGCGAGCCCCAGGACCTGTGTGCAGTAGCCGCGCATCCCGAGCCGGACCACGTCGGAGTTCCTCTGGACCCAGAGATCAAAGCCATGTCGAAGCACCACAGCG
ACGCCGGGACGGCCTTCATTCAGACTCAGCAGCTGCACGCAGCCATGGCCGACACATTCCTGGAGCACATGTGCCGCCTGGACATCGACTCACCGCCCATTAC
GGCCCGAAACACCGGCATCATCTGTACCATCGGCCCAGCTTCACGAGCAGTGAAGACATTGAAGGAGATGATTAAGTCTGGATGAATGTGGCTTCGTTTGAAC
TCTCTCATGGAACCACGAGTACACGCANAACCATCAGATGTACGTGAAGCACGAGAGCTTGTTTCAATCATTCTCTATCGCCGGGCAGTGCCCCTGGCACTAA
AGACTGAGATCGACTGGCTATCAGGGAGCGCACGTGAGTGGACTGAAAGGAGCCACTAAATACCTGGCATGCTACTGTAAAGGAAAAAAATCTGGGTGACTAC
AAAAGCGGGGGAGGGGGGAGATAGGAGATGCTTTTTTGTGGAACAAGGCGACTCGGGGAGAGGGAGGGCCCTGCAAAAAGGGAAC
>‘991108a-096.scf” came from CONTIG 59 at offset 0;
“E:\SEQUENCE\export\EST_db\991108a\991108a-096.scf’ (55>568)
GCACGAGGCTCACGGCCTCTTGCTCACCCATGATATGGTCCCGTTTTCCTCTTGCCGCGTGCACCTCCACCCATTGTCTTGGTGGCACATGGGTGGAACACTT
GATCTGCTCGAGTCTGCCTTCTTCACACATTGCATCTTCAGATTTTCTACTTTTTTGTTCAAAACTAATATTCACCAAGTCAGACTTTGTGTTATTTTATATC
AAGGTATTGGCTGCCAGGGGGTCATCCCTAAGGGGCCTGAAGATGACAAAGGGAATAACAGCACGGGATGTTGGCAAAGATGCYTTCTAGGCTAGAGATCAGN
GGGGGGGAGAGAACTGCAGAAATCACCAACCAGACTGCAGATACGAATCTATGGGCAGGGCTGTGACTGACAGAAGAAACGAGCTGTGGTTGAAGTACATAAC
TCTCACAATACCAGTCTTCCCATCTCCCTCTCANGCANGCCTTTCGATTAGCAATTGCTAAACTTCAAACCAGGCCATCGCCCTGGATCCCAACCC
>‘991115a2-001.scf” came from CONTIG 1 at offset 0;
“E:\SEQUENCE\export\EST_db\991115a2\991115a2-001.scf’ (77>766)
TAATTCGTGCACGAGGGCTGTACTTTATTCCTCATAGTTCCCACACAGCGCCCCGGCCAGTAAGTATGTTCCGGGAAAGGCCTGTGGAGGAAGGCCAGATATT
CAATTGCAAATGTGGGGCAGCCCACAAGGGTGGGGCCCAAGAAAGATTTGAAGGCCACGAGAGAAAGAAAAGCCAAGGATGAACCAGACTTTGATCGGTTACA
AAAGTCATGTAAGGGCAAGGGAAAATGGAGGAACAAGACTAATTCAAGATTGGAAGTTTAGAAACTTTCAAAAGGGCAAGCTTCTCCTTGAAAGGCTTCTTCC
CCAAGAAAGCCCCTTGCTTGCTAAGGGGGGCAGCCTGCAAGTAGCTGCTTGCTGGCTGCTGGCCAAGGTTCCCAGCAAAAAAAGATCACCACTGAAGGTAAAG
AAGGCTCCAGCCCAGAAGGCCTCCTGGCCACAAAGCTGCAGGCCAGAAGGCAGCACCTCCTCCTAAAACTCAAAAGGGGCCAAAAGCTTCTTCCCAAAAGCCA
CCTGCTCAAAGCATTGGGAAGAAAGCATGAGGCATAAGAGGTTTTTAAATAAAATAAAGTTTTTTTGACTGGTGGAAAATTAAAAAAAAAAAAAAGGGGGCCG
GACCCAATGCCCTTTAGGAGGGATTAATTAACGGCGGGTTTTCGGGGATGGAAAACCGGGGACCCAC
>‘991115a2-004.scf” came from CONTIG 2 at offset 0;
“E:\SEQUENCE\export\EST_db\991115a2\991115a2-004.scf’ (76>690)
CTTCATCCTGGACCATGGCAAAGACGGAAAGATCTTTGTCTGGAAAGGCAGGCAGGCCAACACCGAGGAGAGGAAGGCCGCCCTCAAAACAGCGTCCGACTTC
ATCTCCAAGATGGACTACCCCAGGCAGACCCAGGTCTCTGTCCTGCCCGAGGGCGGCGAGACCCCGCTGTTCAAACAGTTCTTCAAGAACTGGCGGGACCCAG
ACCAGNACGGACGGCCCGGGCCTGAGCTATCTCTCCAGCCACATTGCCAACGTGGAGCGCGTGCGCTTCGACGCGGCCACCCTGCAGACCTCCACTGCCATGG
CTGCCCAGCACGGGATGGATGATGACGGCAGAAGGCAGAGCAGATCTGGAGATAGAAGTTCCGACAAAGGCCCGGGGACCCGCCAGTACGGACGTCTACGGTG
GGACAGCTACATATTTTGTCACTACCCCACGGGGGGTCAGGACAGATAATTACATGCAGGCGCCATCCACCAGATGAGGGCTGCCGGCCATCGACGTTATTGA
CAAGAGTGGAGAATCCCGGCAACCGGGGCCAGGAAGACCCCTCACCTTGACGTTGGGCCATAGGGGGCCCCCCGGGGGGAAAGCCCCCCCCC
>‘991115a2-005.scf” came from CONTIG 3 at offset 0;
“E:\SEQUENCE\export\EST_db\991115a2\991115a2-005.scf’ (76>582)
CAAGAAGGGGAATGCCGAGGGAAGCAGTGATGAGGAAGGGAAGTTAGTCATTGACGAGCCAACCAAGGAGAAGAATGAGAAGGGAGCGCTGAAGAGGAGAGCG
GGAGACCTACTGGAGGACTCCCCCAAACGCCCCAAGGAAGCAGAAGACCTTGAAGGGGAGGAGAAAGAGGGGGCCACCTTGGAGGGTGAGAGGCCCCTTCCAG
TGGAGGCGGAGAAGAACAGTAGCCCGTCTGAGCCCGGCTCTGGCCGCGGGCCTCCTCAGGAGGAAGAAGAGGAGGAAGAAGAGGAAGAGGCTGCCAAGGAAGA
CGCCGAGGCCCCGGCCTGAGAGTCACGAGAGCCTGTAGCCACCAAGTTTCAAGAGGAGCCCCTGCCCCGTTCCTGCTGTGTCTGGGGCTACTGGGAAACTGGC
CTGGCCTCCAACTGGGAACCCTTCCCCACCCAACCACTCTTCTTTTCCACTCTCCCCTTTGACTACACCTGAAATGACCCCGAGGGCAGCACGGG
>‘991115a2-006.scf” came from CONTIG 4 at offset 0;
“E:\SEQUENCE\export\EST_db\991115a2\991115a2-006.scf’ (76>588)
CATCCTCTTCCTCTCTGTCTCACGTGTGCTCTTTTTCTTTCTACCAGTGTTATATATTTTAGCAGCATCTAACTCAACATTGATTTCTGCATTTCTTGCTAAT
GCACCTTTAGAAGATACTAGTCTTGGGACAGGATCATTTTGGCCTCATTCCTTTACCACCCCTACACCTAAGAAGCATATGCCAGAAAAATGTAAGAAGCTTT
CAGTATTAGTGATATCATCTGTCACTGTAGGTCATACAATCCTTTTTTAAAGTACTTGGTATTTGGTTTTATTGTTCCCTTTTTTTTCCTGCCTTCTTCTCAA
AGTTCATTCCCCAAAGGGCCCTACTGTACTTCCTGCAGNGCCCCTAGCCCAGAGCCCATGGCTTTGATCCCTCCATCCCCTTGTCTTTTGCTGACCTTGGGAA
TCTTAGTGCATCGCTGGTATTATATGACACTTTCTGGGGAGTGCCCTAAGCTAAATCAAACTGGATTATGGGCAANCAGCGGCTTAAGAAGAA
>‘991115a2-007.scf” came from CONTIG 5 at offset 0;
“E:\SEQUENCE\export\EST_db\991115a2\991115a2-007.scf’ (76>592)
CTGTGGUAAGCGAGAGAAGCGTTGGTCAACTGCCACTCGAATCACGGAGACAGGAAGTCCCTGCCATGCCTCACATCGACAACGACGTCAAACTGGATTTCAA
GGATGTGCTGTTGAGGCCCAAACGCAGTACCCTTAAGTCTCGAAGTGAGGTGGATCTCACAAGATCCTTTGCCTTTTCGAACTCAAAGCAGATGTAGACTGGG
ATCCCCATTATTGCTGCCAATATGGATACTGTGGGCACCTTTGAGATGGCCAAGGTCCTCTGTAGTTTGTCCCTCTTCACTGCTGTCCATAAACACTACAGCC
TCGAGCAGNGGAAAGAGTTTGCCAGCCAGATCCTGACTGTCTTGAGCATCTGGCTGCCAGCTCAGGCACAGGCTCTTTCGACTTTGAGCAGCTGGACAGATCC
TGAACGCTATTCCCCAGNGGAGAATGTATGGCGGGAGCGGGCCAAAGGCTACTCTGTACACTTTTGTATTTGTGAAGGAGTGCGGAAGCGCTTCCTGACACAC
C
>‘991115a2-008.scf” came from CONTIG 6 at offset 0;
“E:\SEQUENCE\export\EST_db\991115a2\991115a2-008.scf’ (82>410)
GCACGGGGGCTAGCTGATGCGCTCAAGGAGAACGACCCTTCCAGCGTGCTTCTCTCCTCGTGGGGTTCCAAGAAGGACCTGAGTACTCCTGCTCAGTATATAC
TGATGGAGAAAGATGCACTCAAGGTGGCCCAAGAGATGAAGGCTGAGTATTGGGCAGGCTCATCTCTCACTGGTGTGTCCAGGAGTTCTTCTTTCGTGTGGCG
GCGCTGACCTTTGAGGTCAACGTGCTGGCTGAGCTGGAGAAATCGGGATCGCGGCGGATAGGGGATGTTGTTCGCATCAACAGTGATGACAGCAACCTCTACC
TAACTGCCCAGAAGA
>‘991115a2-009.scf” came from CONTIG 7 at offset 0;
“E:\SEQUENCE\export\EST_db\991115a2\991115a2-009.scf’ (82>751)
GCACGAGGCTGGGCCTCCTACGCCGGTGCAGGGCCAAGTCTCGGCCGACTTACAACAAGGTAGCTGTCACAAGGGCGGGGTTACCCTCGGGTTCCTGTGCGGG
TTCATTCCCCCCAAAAAGAGTCGATGGGACCAGACCACAAGGCTCTCCCAGGGGACCAAGGGGGAAGGGACCGGGATCCCAGGGTGTGGGATTGCCAAAACAA
CCCGGGGGATGGCTTCAAGGAACAAGCGCCCATGCCTGGAAATAACCTGGAAAATCGCCCCAAGGACCCTGGAAAATGTACTGGGGATCAAACTATTTTTCGA
GAATAATAAAAAAAGGAAACAGAATCTTTTGGGCTTGGGCGTTTGATTGCCCTTTGGACTTAAATATTTTTTGTAAAAAAGAAAAATAAAATTGACCCCCAAA
GATTGGTCTTTCCCATGGTAGCGAAGATTGGAACATCTTTTTTTAATGACAAAAAAATTTGTTTATAAAACCCCATCAAACAAAAAGACTTCCTTGATTTTGT
GTTCTTACCCCCCCCCCCCGGGGGATCAAAAAGCGGATCCTTGAATTTTGCTTGGGGAAACCATGAACTTGTCATGCCCCCCCGGAAGCCCCCACCCTTGAGG
GGCACAAATAAAAGCCCAACCCGGAGGAAAAAACCACAAAACCGG
>‘991115a2-011.scf” came from CONTIG 8 at offset 0;
“E:\SEQUENCE\export\EST_db\991115a2\991115a2-011.scf’ (83>521)
GCACGAGGCCGTACGCTCCCTGCACTTCGGGTCTGCTTGCTCCAGCCCGCTCTGCCGCCGCCGCCGTCGCCGCCATGGGCCCAAGCTGACATTGCCCTGATTG
GACTGGCTGTCATGGGCCAGAACTTATTTTGAACATGAATGACCATGGCTTTGTGGTCTGTGCTTTTAATAGGACAGTCTCCAAGTTGATGACTTCTTGGCCA
ACGAGGGCGAAGGGACCAAGGGCTTGGTGCTCACTCCTTGGAGGAAAGGCGGCCAAGCTGAAAAAACCACGCGGGATCATCCTCCTTGGGAAGGCGGGAGGCC
GTGGATGATTATTGAGAAATGGTACCTTGCTAGAATTGGTGATATATTATGATGGAGAAATTTGAATACGGATACAGAAAGGGGGGAGACTCAGAAAAGGAAT
TGTTGGGGGGGGAAGTAGGGGGAAG
>‘991115a2-015.scf” came from CONTIG 9 at offset 0;
“E:\SEQUENCE\export\EST_db\991115a2\991115a2-015.scf’ (75>420)
TTGAGTTCTTTCCTCACCATGTCTTCTCACAAGACTTTCAGGATCAAGCGATTCCTGGCCAAGAAACAAAAGCAGAATCGTCCCATTCCTCAATGGATTCGAA
TGACTGGCAATAAAATGAGGTACAACTCCAAGAGAAGACATTGGAGAAGAACCAAGCTGGGTCTATAAGAAGCAAGCTGGGTCTATGAGAAGTGGTCTTAACA
TGTAGACCACTTTTTTAAGCAGCCAGATCACAATGAAAACATCACTACTGTAATGCTTGGCCCATGATGTTATTTCCTCACTATCAGTCTGAGACCCAGCAAT
AAATATAAAACGTTGCAAAAAAAAAAAAAAAAAACTCGAGGGGGGGCCCGGAACCCAANTCGCCTATAGTGAGTCGGATTACAATTCACTGGCCGGCGGTTTA
CACGCGCGGGACTGGGAAAACCCTGGCGTTACCCACTTATNGCCCTGCAACACATCCCCCTTTGCCGCTGGGGNATAGGGAAAAGCCGCACCGACGCCCTCCC
ACAGTTGGCACCCGAAGGGGAAGGGAAATGTAGCGTTATATTTGTTATATTCGGTAATTTTGTTAATAATTCTTTTTACCAAGCGAAAGGGCAAACCTTTAAA
AAAAAAAACGGAAGGTGGGGGGTCCGTGGGACAAAACCTTTAAAAAGGGCTCCGCCGGGGAA
>‘991115a2-050.scf” came from CONTIG 9 at offset 326;
“E:\SEQUENCE\export\EST_db\991115a2\991115a2-050.scf’ (82>801)
ACAAAACTGGAGCTCCACCGCGGCGGCGGCCGCTCTAGAACTAGTGGATCCCCCGGGCTGCAGGAATTCGGCACGAGGAAAGGGATTCTATCCTAATAACAGG
TCTCTTCTCCCACCTTAACGGCGCCTCCCCTCAGCACCCACCTGTCCCCTCTCCGCAGTTTGCATATAGGAACGTGGGCCATCCTGCCGTTGCGTGTACTTGC
GGGTCAACCACCTTCGTGGGAACCCATTGTATTGCCAGGGGCTGAAGCAAAGTAGCCCCTCCCTTTCCTCCACTGATGCCCAACAAGAACCTCCCTCCAGTCT
CAGTCTCAGTCACTCTTGTTTTCTATGGAAGGGAAAAGGTTTGGTCTACCTAACAAGTAGGCAATGCGAATGGGCCAGTATATCCAGTCCTTTAGGCAAAAGG
AAAATGCATTCTACCTTCTCCTGCCCCCTCCTTTCCTTTTAAACAAAAAAGGAGATACAACCTCCTGTGCCAAAGTAATGGGTCCATTCAGAAGGTGGAGAAG
CATCATCCGTTGGTTAGCTATTGAGGTTAGGACCACTGACCCTCCCGGGGAAGGATGCAATGATTTGTGACAGTCCTCAGAAAATTTGATATGCAGATGAGGC
CGAAATTTTCTCATAGGTATTATTGACTAAGAGATAAATTTTTTTATCTTGGGCGGTGTTCTTAATTTGACGGGCGGTGGCTCCCACCAACCTTTTTTTCTTT
TGGGTTTTACCAGGTTTAACCCAAGTTTTTTTTTTCAAACCCTTTCTTACTTTTTTTTTTTTATTGC
>‘991115a2-016.scf” came from CONTIG 10 at offset 0;
“E:\SEQUENCE\export\EST_db\991115a2\991115a2-016.scf’ (76>673)
TAAGAAGATGCCTGCACACAAGACTAAAAGTTCAGTGATCTTATGCCCAGATGGACAGTCCCAGGCCCGGTGGTTCTACCTGCTGCAAGCTGCCCACTGGAAA
GTATGGCTGCTGCCCGATGCCCAATGCCATTTGCTGCTCCGACCACCTGCACTGCTGCCCCCAGAACACTGTGTGTGACCTGACCCAGAGTAAGTGCCTCTCC
AAGGAGAACGCTACGGACCTCCTCACCAAGCTGCCCGCACACACAGTGCAGGATGTCAAGTGCGACATGGAGGTGAGCTGCCCAGACGACTACACCTGCTGCC
GCCTACAGTCCGGGGCCTGGGGCTGCTGCCCTTTTGTGCAGGCCGTGGGCTTGGAGGACCATGTGCACTGCTGGCCGTCCGGTTTAGGTGTGACACAGAGATG
GTGGGTGTGAGCAGGGGACCCGCCAGTGCCGGGATGAAGAAGCCCCACCACCTCACCCGCGGACCTCGAAGCGCGGGGGGGAGCCCCTGGATAACGCACAGTG
TCTTTTTCCTACTGTGCGATTAAGCGGGAGGGCCTGTGCCTGTCCAAGTGCTGCTGTCGACCCCCCTGTGCCAAGGCTACGGG
>‘991115a2-018.scf” came from CONTIG 11 at offset 0;
“E:\SEQUENCE\export\EST_db\991115a2\991115a2-018.scf’ (82>549)
GCACGAGCTGGGTCGGCAGCGGCAAGACTGTTTGGCCGCCTCTACAAGGTCCCGGCCATGTTCAGCTCCAGTGCCAAGATTGTCAAGCCCAACGGCGAGAAGC
CGGACGAGTTTCGAGTCGGGGATCTCCCAGGCCCTGCTGGAGCTGGAGATGAACTCGGACCTCAAGGCGCAGCTGCGGGAGCTGAACATCACGGCCGCCAAGG
AGATCGAAGTTGGCGGTGGCCGGAAAGCTATTATTATCTTCGTCCCCGTCCCGCAGCTGAAGTCTTTCCAGAAAATCCAGTGCGCCTGGGCGCGAGCTGGAGA
AGAAGTTTAGGGGAGCACGTCGTTTTATTGCCGAGAGTATAATCTGGCTAAACCACTCGAAAAACCCGACGAAAATAACAAAACGTCCCAGACCGCACTTGAC
GCTGGCACCACCCCTTTGTAGATTGGTTTCCAGGAAATGGGGAAAAGATCGGGGAGT
>‘991115a2-019.scf” came from CONTIG 12 at offset 0;
“E:\SEQUENCE\export\EST_db\991115a2\991115a2-019.scf’ (77>614)
GACAGTTCATAGTGGTGAGAAACCTTACAAATGTGATGAATGCGGCAAGGCCTTTCCTATAAAGTCAACCCTTTCACAGCATCAGACAATTCATACTGGTGAG
AAACCTTACAAATGTGATGAGTGTGGCAAGGCCTTTCGTTTAAAGGCAGTCCTTTTAAAGCATCAGACAATTCATACTGGACAGAAACCAAACAAATGTGACG
AGTGTGGCAAGGCCTTTCGTGTAAAGTCAACCGTAAAAAAGTCATCAGGCAGTTCATACTGGTGAGAAACCTTACAAATGTGATGAGTGGGGAAGAGTCTTCC
GTAAAAAACCACAGCTTCCACGTCACTGCAGAATTCATACTGGAGAGAACCTTTTATATGTAAGAATGGGGCAGTTCTTCAGTCAAACACACCTTATAGACAT
CGAGATACTATAGAAAAACTTCAATGTTTGAGGGGGAAATCTTTATCAGGCACACACTCCTAAAATAAAAATCCATACTATAAAAAATATGGAGTGGATTAAA
NAAATTTAATTCAAGTGAAAG
>‘991115a2-021.scf” came from CONTIG 13 at offset 0;
“E:\SEQUENCE\export\EST_db\991115a2\991115a2-021.scf’ (76>670)
TTCCCAGGTGCTGCTGGCCGAGTCGGTCCCCCCGGCCCCTCTGGAAATGCTGGACCCCCTGGCCCTCCTGGCCCTGCTGGCAAAGAAGGCAGCAAAGGCCGCC
GCGGTGAGACTGGCCCCGCTGGGCGTCCCGGTGAAGTCGGTCCCCCTGGTCCCCCTGGCCCCGCTGGTGAGAAAGGAGCCCCTGGTGCTGACGGACCTGCTGG
AGCTCCTGGCACTCCTGGACCTCAAGGTATTGCTGGACAGCGTGGTGTGGTCGGCCTGCCTGGTCAGAGAGGAGAAAAGAGCTTCCCTGGTCTTCCTGGCCCC
TCTGGTGAACCCCGCAAACAAGTTCCTTCTGGAGCAAGTGGTGAACGTGGCCCCCCTTCCCATGGCCCCCCTGGATTGGGTGGACCCCCTGGCGAGTCTGGAC
GTGAGGGAGCTCCTGGTGCTGAAAGATCCCCTGGACGAAATGGTTCTCCTGCCGCAAGGGTGACCGGGGGGAGAACGGCCCTGCTGGACTCCTGTGCTCTGGC
GCTCCCGCGCCCCCGCCCTGTGGGACTGCCGGCAAGACGGGGATGGGGGAGAACGCCCTGTGGTCTGTGTCCCATTGC
>‘991115a2-084.scf” came from CONTIG 13 at offset 0;
“E:\SEQUENCE\export\EST_db\991115a2\991115a2-084.scf’ (78>522)
CCTGGATTGGCTGGACCCCCTGGCGAGTCTGGACGTGAGGGAGCTCCTGGTGCTGAAGGATCCCCTGGACGAGATGGTTCTCCTGGCGCCAAGGGTGACCGTG
GTGAGACCGGCCCTGCTGGACCTCCTGGTGCTCCTGGCGCTCCCGGTGCCCCCGGCCCTGTCGGACCTGCCGGNCAGAGCGGTGATCGTGGTGAGACCGGTCC
TGCTGGTCCTGCTGGTCCCATTGGCCCCGTTGGTGCCCGTGGCCCCGCTGGACCCCAAGGCCCCCCGGGGGAGAAGGGTGAGAAGAGCGAACAGGGCGACAGA
GGCATTAAGGGTCACCGGGGCTTCTCTGGTCTCCAGGGCCCCCCGCCCCTCCCGGCTTCCTGTGAGCAAGCCTTTCGGAGCTCTGTCCTGCTGGCCCGCGGCC
CCCCTGGCTTTGTGTTTTCCCGAAAAATGACTC
>‘991115a2-056.scf” came from CONTIG 13 at offset 499;
“E:\SEQUENCE\export\EST_db\991115a2\991115a2-056.scf’ (76>528)
TGGACCTCCTGGTGCTCCTGGCGNCTCCCGGTGCCCCCGGCCCTGTCGGACCTGCCGGCAAGAGCGGTGGATCGTGGTGGAGACCGGTCCTGCTTGGTCCTGC
TGGTCCCAUGGCCCCGTTGGTGCCCGTGGCCCCGCTGGACCCCAAGGCCCCCGTGGTGACAAGGGTGAGACAGGCGAACAGGGCGACAGAGGCATTGGGTCAC
CGTGGCTTTTCTGGTCTCCAGGGTCCCCCCGGCCCTCCCGGCTCTCCTGGTGAGCAAGGGTCCTTCCCGAGCCTCTGGTCCTGCTGTTCCCCGCGGTCCCCCT
GGCTCTGCTGGTTCTCCCGGCAAAGATGGACTCAATGGTCTCCCAAGCCCCATCGNTCCCCCTGGGCCTGAGTCGCACTGGTGATGCTGTTCCTGCTGGTCCT
CCCGCCCTCCTGGACCCCCTGGGCCCCAGNCCCTCCAG
>‘991115a2-085.scf” came from CONTIG 13 at offset 580;
“E:\SEQUENCE\export\EST_db\991115a2\991115a2-085.scf’ (83>661)
GCACGAGGGCTGGTCCTGCTGGTCCCATTGGCCCCGTTGGTGCCCGTGGCCCCGCTGGACCCCAAGGCCCCCGTGGTGACAAGGGTGAGACAGGCGAACAGGG
CGACAGAGGCATTAAGGGTCACCGTGGCTTCTCTGGTCTCCAGGGTCCCCCCGGCCCTCCCGGCTCTCCTGGTGAGCAAGGTCCTTCCGGAGCCTCTGGTCCT
GCTGGTCCCCGCGGTCCCCCTGGCTCTGCTGGTTCTCCCGGCAAAGATGGACTCAATGGTCTCCCAGGCCCCATCGGTCCCCCTGGGCCCTCGAGGTCGCATT
GGTGATGCTGGTCCTGCTGGTCCTCCCGGCCCTCCTGGACCCCCGGCCCCCCAGTCCCTCCCAGCGGCGGCTACGACTGAGCTCCTGCCCCACCACCTCGAAA
AGCTCAGATGGTGGCCGCTATACGGCTGATGATGCCAAGGGCCGGGCACGGGACCTAAGGGGAACCACCCTAAACCTGACCACAAAAGAAACACCGAACCCGA
GGCACCGCAAACCCGCCCCCCCGCCGGACTAAAAGCCCTTTTCGGAAAGAAAAATGATTGCC
>‘991115a2-090.scf” came from CONTIG 13 at offset 624;
“E:\SEQUENCE\export\EST_db\991115a2\991115a2-090.scf’ (84>340)
GCACGAGGCTTGGACCCCAAGGCCCCCGTGGTGACAAGGGTGAGACAGGCGAACAGGGCGACAGAGGCATTAAGGGTCACCGTGGGCTTTCTCTGGTCTCCAG
GNGTCCCCCCGGCCCTCCCGGCTCTCCTGGTGAGCAAGGTCCTTTCCGAGCCTTTGGTCCTGCTTGTCCCCGCGGTCCCCCTGGCTCTGCTTGTTCTTCCGGC
AAAGATGGACTCAATGGTCTTCCAGGCCCCATCGGTCCCCCTGGGCTTGGAG
>‘991115a2-022.scf” came from CONTIG 14 at offset 0;
“E:\SEQUENCE\export\EST_db\991115a2\991115a2-022.scf’ (83>568)
GCACGAGGGCCCAGAACGTGCTGTCGAAGGCGGACGTGATCCAGGCCACCGGAGACGCCATCTGCATCTTCCGGGAGCTGCAGTGTCTGACGCCCCGAGGCCG
CTACGACATCCGCATCTACCCCACCTTCCTGCACCTGCACGGGAAGACCTTCGACTACAAGATCCCCTACACCACGGTGCTGCGGCTCTTCCTGCTGCCCCAC
AAGGACCAGCGCCAGATGTTCTTTGTGATCAGCCTGGACCCCCCCATCAAGCAGGGTCAGACTCGCTACCACTTTCTCATCCTGCTCTTTCTCCAGGACGAGG
ACATCTCCCTGACGCTCAACATGAACGAGGAGGAGGTGGGAGAAGGCTTTGAGGGCGGGTCACCAAGACATGTCAGGATCCCTCTCGAGAGGGCAGCCGGTCA
TGAAAGGCTGGTGACCGCAGATCAGGGCCCGCCATTTTCAAGGCACTGGGGGCCAGGCATCACTGTCCACAGGC
>‘991115a2-023.scf” came from CONTIG 15 at offset 0;
“E:\SEQUENCE\export\EST_db\991115a2\991115a2-023.scf’ (82>608)
GCACGAGCGCTGGGCCGCCGCCATGTCTCTAGTAATCCCTGAGAAGTTCCAGCACATCTTGCGAGTACTCAACACCAACATCGATGGGCGGCGGAAAATTGCC
TTTGCCATCACTGCAAGGGTGTGGGGCGAAGATATGCTCATGTGGTGGTGAGGAAAGCAGACATCGACCTCACCAAGAGGGCCGGGGAGCTCACCGAGGATGA
GGTGGAACGTGTGATCACCATTATGCAGAATCCACGCCAATACAAGATCCCAGACTGGTTCTTAAACAGACAGAAGGACGTGAAGGACGGGAAATACAGCCAG
GTCCTGGCCAACGGTCTAGACAACAAACTCCGTGAAGACCTGCAGCGCCTGAAGAAGATTGGGCCCACAGGGGCTGCGCCACTTCTGGGACTCCGGTCCGAGC
CAGCCACCAAGACACAGCCGCCGGGGCGCACTGGGGGGGCCAGAAGAAAATGGTGGCTTGGTTATAAATATTATAAGTAAAAAAAAAAAAAATGAGGGGGCCC
CACCATTC
>‘991115a2-024.scf” came from CONTIG 16 at offset 0;
“E:\SEQUENCE\export\EST_db\991115a2\991115a2-024.scf’ (76>606)
CGGCGGCGAGATGTCCGGAGGATCAGCTAGGAGCCTGGGCAAGGGAAGCGCCCCCCCGGGGCCCGTCCCGGAGGGGCCTGATCCGTGTCTACAGCATGAGGTT
CTGCCCGTATGCCCAGAGGACTCGCCTGGTCCTGACGGCCAAGGGTATCCGGCATGAAGTCATCAACATCAACCTGAATAAGCCTGAGTGGTTTTTCAAAAGA
ATCCCTCAGGCCTGGTGCCGGTTCTGGAAACCAGTCAGGGTCAATTGATCTGTGAATCTGCCATCACTTGTGAGTACCTGGATGAAGCATATCCAGNGAAGAA
GCTGTTGCCAGGCGACCCCTATGAGAAAGCTTGCCAAAAGAGGTCTTGGAGTCCTTTTCTAAGTACCACCTTTGATATTGAGATCTTATATACAAAATAAGAA
GATGCTCTGGCTAAAGAAAAATGATAAAGAATACCAACTAAGNAGGTCTGATGAAAGAAAAAACTTCTTGTGGCATTTTTTTTTATGATACTATTGCCCTGGT
GAAGCTGGAGC
>‘991115a2-025.scf” came from CONTIG 17 at offset 0;
“E:\SEQUENCE\export\EST_db\991115a2\991115a2-025.scf’ (77>511)
CGCCGGGGGGCCATGGGACGGNGGCCGCTGATGGGAAGGGGCCGCGTGTGTTTTTGCAGAAGTCAGCGGTTTCGGGGACCCAAAAGATGGGAGGGAAGAAACC
TGGNGGCGGNGTCTTCTTATCCCGGGCCCAGTCNCCCCCACGGTGGTNCGCGGCGGGCTTCGAGTGTCTTTTGTACCACAAAGAGGGTCTTAGCTCTTGGAGG
GTGGGGCGAGGGGGAGGCTCGCGGCCGCGCTGGGACTGGCAAGGGTGGGATCCCGTCTGGGGCAGAGTGTGTGGAGGCGTGGGCCCCCCGTGGGGCAGAGGGA
GAAGCCCCCCCCTGTAGCCTGGGCGGCCCGCAGCCTTTTCTGCCTTGGATACCCGCACAACAGATCCCCACTTTGCTGTGCGGGGCGACGCGGCCCGGCCCAC
GGCAGACCTGGGTGGCAACAGCT
>‘991115a2-026.scf” came from CONTIG 18 at offset 0;
“E:\SEQUENCE\export\EST_db\991115a2\991115a2-026.scf’ (83>640)
GCACGAGGTTTTTTTTTAAAAAACAAATAAAAGGTTTATTCATAAGCAGGAATGCTGTCCAGAATACATACAGTGCAAACTCTCCTTGTCCTTGGGATCAACA
CAAGCCCACAAGCCAGGAAAATTGTTTATCAGTTAGGTCAAATGGACTCCCCCCAGCACTTGGGCCCTAGCCTGGCCAAGGCCCCCTTCCTTGGCAGAGAAGG
TTCTTTTTCTGTTTGGGTTCACACAACCAAACCCAAAATGGAAGCTGAGATTAGAGCAGCACAAGCTTTATTCATTGGTCAGAGAATGGAGAAGTGGGAACTT
ATTAACGTTGATAAACAAACTTCTCGCCTTAATGTGGAGCCAGATTTGCTGTTAAGAGCATGNGGGAAAGAGTGCGAGAATGGGGGGGAGCAAAGCTCTCGTT
TCTGGGAAGAGCAGGGTTTCCTTAATGAGNGCCACTTTTTTGCCTTTTTCAGGATTCTGTCACGAACAGAGNNANCATTATACTATGAGAGTTAGTCATCACC
GTAGCTGTCGATTTGGACCCAGATGACAGCAGCTCCGTCTC
>‘991115a2-027.scf” came from CONTIG 19 at offset 0;
“E:\SEQUENCE\export\EST_db\991115a2\991115a2-027.scf’ (76>628)
CTATTCTCAACCAACCATAAAGATATTGGTACCCTTTATCTACTATTTGGTGCTTGGGCCGGTATAGTAGGAACAGCTCTAAGCCTTCTAATTCGCGCTGAAT
TAGGCCAACCCGGAACTCTGCTCGGAGACGACCAAATCTACAACGTAGTTGTAACCGCACACGCATTTGTAATAATCTTCTTCATAGTAATACCAATCATAAT
TGGAGGATTCGGTAACTGACTTGTTCCCCTAATAATTGGTGCTCCCGATATAGCATTTCCCCGAATAAATAATATAAGCTTCTGACTCCTCCCTCCCTCATTC
CTACTACTCCTCGCATCCTCTATAGTTGAAGCTGGGGCAGGAACAGGCTGAACCGTGTACCCCTCCCTANCAGGCAACCTAGCCCATGCAGGAGCTTCAGTAG
ATCTAACCATTTTCTCTTACACTTTACAGAGTTTCTCATTTTAGGACCATCAACTTATTACACAATATTAAATAAGCCCCCGCATGGACATACCAAAACCTTG
TGTTGATCGTATATTACGCGTACTATACACCTCGTCCT
>‘991115a2-043.scf” came from CONTIG 19 at offset 4;
“E:\SEQUENCE\export\EST_db\991115a2\991115a2-043.scf’ (73>538)
TCTCAACCAACCATAAAGATATTGGTACCCTTTATCTACTATTTGGTGCTTGGGCCGGTATAGTAGAGAACAGCTCTATAGCCTTCTAATTTTGCGCCTGAAT
TAAGCCCAACCCGGAACTCTGCTTCGGGAAACGACCCAATTCTACAACGTGAATTGTTAACCGCACACGCCTTTGTAATAATCTTCTTTATATTAAATACCAA
TCATTAATTGGAGGATTTGGGTAACTGACTTGTTCCCCTTATAATTGGTGCTTCCGATATTATATTTCCCCCAATAAATAATATTAACTTTTGACTCCTCCCT
CCCTCTTTCCTACTACTCCTGCCTTCCTTTTTTTTGTAACTGGGGCCAGAACAGGTGAACGCGTACCCCTCCCTTACAGGCAACCTACCCTTGCAGGAACTTT
ATAGATTTAACCTTTTTTTTTCTCTTATAAGATTTCTTTTTTTTTGAACATTCA
>‘991115a2-028.scf” came from CONTIG 20 at offset 0;
“E:\SEQUENCE\export\EST_db\991115a2\991115a2-028.scf’ (82>595)
GCACGAGGTTAAGTTAAATATAATTGAGATAAGAGTTTTGGGAGTTGAGCTCTTTGGGAATAACTGTTTTAGGAAATCTACTTAAAAAAAGTCTCTCCAGATT
TTGGCAACTTGAAAGTTTTGTTAAGTTAAATAATGGGGAGTCACTGTCTGAATTGCTAAACAAGCTAAACAAGTCTAAGTTCACCCCCCTTTTTTATGAGAGA
AAAACTAAATATGTTTGGGTTTGTTAGAAACATATGACACCACACTGAGGACAGAAGTTATTCGATGAGATGACGTGTGTTTCTGAAGACTGTAGAAACATAA
GTTTCCAATCAGGAAGCCCTGGTATGATCACAAGTCCTTGCTTCTGTTTTGCTGGAGATCAGGGTTCTTAAGGCTGTGTTGTTTAGTGCTCGGCACCTCCGAT
CTTTTGNGACCCATGGACGTACCCCCCAGGCTCTCTGCCATANATTCTCAGGCAGAAACTGAAGGGTGCCGTTCCTTTGCGGGGACTCCCCCCCAGGTCCACA
G
>‘991115a2-030.scf” came from CONTIG 21 at offset 0;
“E:\SEQUENCE\export\EST_db\991115a2\991115a2-030.scf’ (76>661)
TTTGGAGATACATGGAAGCTTTACTGGACAGGGTGGAGGGGCTGCCAGCGGGGGGCGGGGGGCAATGCAGGCAGGGCAGGGGTTGTGGCTCTTGACCTGGAGA
CCCCGGCACGTGGCCGCTCCCAGCACTGACGGCGCCTTCCCTGGGAGCTTGCGGCTTCTCTGGAGGGCGTCCCTTCACCAACCAACAGCCAGAGHCCTCTCCT
CCTGGAACTGTGGGGGGCAGTGAGTGCCATGTTGGAGGGCGAGGTCGGAGGGCCCCGCAGCAAGAGCTGCCAGGGTTTCGGNGCGAAGGCAGGCGTGAGAAGC
AGCTTCTTTCGCAAGGTCGGNTTCGGGGGGCGGNGGGCGGATCTGGCCGGGTTCTCCCCTGGGGCGGCGGACGGGGAAAGCAGACCTGCTGCTGNCTTGCCCN
TGCTCCTGGAAAGAGAAGAGAGCCTATGCGCCGAGCCTCGTCCACAGCACTTCTTGTATTCCGTGTCCGCGGCCCAAACTGTACCTCTCCCAACGGGTGCTTT
GACGTAAACATTTGGGCCTCCTTTGTTTCCAAAACCGTTGTTATGTTCGCCCCATATCTTGAGGCGGA
>‘991115a2-031.scf” came from CONTIG 22 at offset 0;
“E:\SEQUENCE\export\EST_db\991115a2\991115a2-031.scf’ (82>699)
GCACGAGGGCGGGCGCTTTTTCTGCCCGCGGTGTCTCAGATTCATTCTTAATGGAACTGAGAACTTAATCTTCCAAAATGTCAAAAAGACCATCTTATGCCCC
ACCTCCCACCCCAGCTCCTGCAACACAAATGCCCAGCACACCAGGGTTTGTGGGATACAATCCATACAGTCATCTCGCCTACAACAACTACAGGCTGGGAGGG
AACCCGGGCACCAACAGCCGGGTCACGGCGTCCTCTGGTATCACGATTCCAAAACCCCCAAAGCCACCAGATAAGCCGCTGATGCCCTACATGAGGTACAGCA
GAAAGGGCTGGGACCAAATAAAGGCTTCCAACCCTGACCTAAAGTTGTGGGNAGAYGCCAAGATATTGTGGGATGTGCGAGATCTCACTGATGAAAAAAAAAA
ATATTTAACGAAAACCAGCAAAAAGATGATACNATGATCTATGAGGCCTACATAATCCCCCGCACCTGCTACTAAAGCAAAGAGCGCAGAGCGCTTTAAGAAA
AAAAGAAGAAACGCGGCTGAGAAGAGACCTACTGAATCACCGCTGAACCAAAATTTNAGAGTTTTATGAAAAAACCCGCCCTCCAAAAACCCCCCAGAACCT
>‘991115a2-032.scf” came from CONTIG 23 at offset 0;
“E:\SEQUENCE\export\EST_db\991115a2\991115a2-032.scf’ (83>484)
GCACGAGGCAGTCCCTAATTGAGGAACTAAGATCCTACAAACTGTGGGGTGAGGCCTAAGAAAAAAAAGAAAAAAAAATGGGTGGTCAAGGAGGGCCCTTTCT
GAGGGAGTGACATTTACGCAGAGATGTGAACAAAGTGAGGGTTTTTCCTGGGGAAACACTTGCCAGGCACAAGTGTGATCATGCTTAACATGTTCGCTAGCAA
GGACTGTAGGGGAATATTTATGAGGAACGCATGTGCCACAAGAACAGAGATCCTGTTCACCTATAATGCCTTGAGCAGACCTGGCATGTACCCTCAACAATAC
TTGAATGAATGGATGGGCAGATAGATGGATTTTAATGAATGAGCAATAATGATACATTCTCCCAATAACCTGGAGCCAGAAGAGAGGAGA
>‘991115a2-033.scf” came from CONTIG 24 at offset 0;
“E:\SEQUENCE\export\EST_db\991115a2\991115a2-033.scf’ (76>595)
CTCACCTGCCACCTATGGGCCACCTGAAGCAGTGCCACGGCCAGGAGGAAGCTGGCAGGCGCAAATTGTGGTCAGCCCCTGCTGGGCTGCTGCGGGGGGACAG
GTCCTCGCCTCTGCTGAGTCCGCTGAAAGCCCCAGACTCCCTGCCCCAGGGGACTCCACTGGCAGCCAGCCTCTCTGCAGCCTCCCTAACACCCTTATGTGTC
TCAAAAGAGGGGAAGTGCCCTCTGTTCAATGCCCCCAGCCATGCCCAGAGCTCAAGCCAAGCTCCTGCAGGGACTGGCGTGCCCTCCCCAGGCCCCACGTGCA
TGGACGCTTCCTAGTGCGTGCCGGCTCCTTGCTGAAGGCTACAGAGAAGAGGGGNCCTAATCAGGCCTGGTGAATGCCTGCCCCTGGAAGCCGCCAGGCTGCA
GAGTTCCAGAGACTCTGGGACTTTGAGAGAAGNTTTCAAACCCAGCCTACAGGGACGACGCCCCACTCCTTGCTGGCCCGGCTCACCCTCCCAGGAGGGGGCT
CCGG
>‘991115a2-034.scf” came from CONTIG 25 at offset 0;
“E:\SEQUENCE\export\EST_db\991115a2\991115a2-034.scf’ (75>554)
TGGACGCACTCGGGAATTGTAGAAGGACGAGGCTCAGCTCTTGCCAGGCCGACTTGAGACATGTCTGACACAAGCGAGAGTGGTACGGGTCCAACCCGCTTTC
AGGCTGAAGCTTCAGAAGAGGACCCTGGCTTGAAGATGCAGACCGGACTGACAGGGACCCAGAACTTAAAGGCCTCAGAAACACCGAAAGGCTCAAAGACACC
AGAGGGCTCAAAGGCCACGAAGATCTCAAATGCTGCAGGCGTCTCAAAGGCCACTGAAGCTCAGGAGGTATCTGCCACTCAGGCTTCACCTACCACTAAACTG
ACCGATACCCAGTTTCTAGCAACCAAAAGAAGAGTCTGGCAGTTGACACCAAATGCAGCATACTGACCCTTAGGCTGTGAAATGCCTGGTATGAAACCAAAAG
GTAGTTTGGGTTGATACCAAGGTCATACAAAACCCTGGAAATGAATTTACTGCTTTAGCTTTGGCGA
>‘991115a2-035.scf” came from CONTIG 26 at offset 0;
“E:\SEQUENCE\export\EST_db\991115a2\991115a2-035.scf’ (82>672)
GCACGAGGAGAGAACTGCCACAGGGGATTTGCACGGGTAAGTGCTATATATGTGAGGTACCTACATGGCTGAATAAAACCAATTGTATGAAATCATTTATGCA
AAATGGAAAGGGTGATTGGGGTGAAAATCTGAAGGAGAGATTGCTCGCTGTAAGCAGCTCATCTGCGATCCCAGCTATGTGAAAGATCGAGAAGAATAAGTGG
GCCAGGTGATCAGAGTCATCTGTATTCTCAGCCACCCCCATCAAAACACCAATGACGCCCACTCCTGCCAGATCATTATTCGACAGACCCAAGTCAACCGGAA
GTCAGATATCTACGTCTGCATGATCTCCTCTGCACACATGTGGCCGAACAGGCAGAACATCGCCATTGCCACACAAAGGNGGAACCAAGAGGCCGAAAGGAAA
CAAACAACCTGGGCTTTTGGACCAATGAACAGAATTCGTAGATCAGGACCTCCTGACCAAAGATGGGAAAAAAGCAAACTTTTTCGCACTTAAGCACATCCTT
TAAACACTGGAGACATAGAATTATAAGAAGAGGACCGGNTGCTGGAAAGAAGAAAAAGAAAAAGGGAAAAAAAGAC
>‘991115a2-036.scf” came from CONTIG 27 at offset 0;
“E:\SEQUENCE\export\EST_db\991115a2\991115a2-036.scf’ (83>509)
GCACGAGGGCCAAGCCCTCCTCCAGGGGATCTTCCCAATCCAGGGATCAAACCAGGTCTCCTGCATTGCAGGCGTTTTCTTTACTGGCTGAGCAGGTGGGAGA
AAATAAACACAAAGATTACTGAATGGCCACATAGGCTAAGAAATGAAATAGGTGTAGTGGGGCGGAGCGGGGCATAAAAATACACGGTGCCCGGGTTTTGGTC
ACTTAGTGGAGGTGCAACGGGCAGTCACTGGGCAAGATCCTGGGGTCACAGCGGGAAAGCGGGGTCCCTGCTCTCACTGACTCACGTTCTAAGGGGGGCGGGG
CGGGGGGCGGGGAGGGCAGGCTGGAGGAGAGAGTTTGGGGGACCAAAACAAGCACGGGCCAGGGCTGGCAGGATGCCTGCGAAAACGGCGGGGTGCACACCGA
GCTGGGGCCAGATCG
>‘991115a2-037.scf” came from CONTIG 28 at offset 0;
“E:\SEQUENCE\export\EST_db\991115a2\991115a2-037.scf’ (236>332)
AACAAACCAATCATTTACAACGCTTTATCCCATTGTTGTCTCGTCAGAAAAGTGAAACGAACCTCACAAAAATTCAATATTATAGGAGCTGGAGAAG
>‘991115a2-038.scf” came from CONTIG 29 at offset 0;
“E:\SEQUENCE\export\EST_db\991115a2\991115a2-038.scf’ (75>544)
CGGTGCTGTCCGGCGGGGACCACCATGTACCCCGGCATCGCGGACAGGATGCAGAAAGAGATCACTGGCCTGGCACCCAACACAATGAAGATTAAGATCATCG
CGCCCCCTGAGCGCAAGTACTCCGTGTGGGATTGGCGGGTCCATTCTGGCCTCGCTGGCCACCTTTCAGCAGATGTGGATCAGCAAGCAGAAGTACGATGAGT
CCGGCCCCTCCATTGTTCACCGCAAAAGCTTTTAGCGGACTGGTAGCTGCGTTACACCCTTTTTTTTGACAAAACCTAACTTGCGCAGAAAACGAGATGAGAT
TGGATGTGTTTATTGTTTTTTTTTTTTTTTTTTTTGTTTTTTTTTTTTTGGTGCTTGTATTAGATTTAAAAATGGACGGGAAAGAGAAGAACGGGTGGATGAG
ATTCCCAAATTTTAAGGGGGCGGGACTGATTGAATGGTTGTTTTTTTTTATATATTT
>‘991115a2-039.scf” came from CONTIG 30 at offset 0;
“E:\SEQUENCE\export\EST_db\991115a2\991115a2-039.scf’ (76>691)
GCACGAGGTCGAGTTTTTTTTTTTTTTTTTTTTTTTCCAGGTCAAGAAAAATTTTATTGAGAAACCAGGGACACAGCCATAAGAGAGGGAAGCACACAGGACT
GCAAACTAAAACCCAATAGCCAGCAAGGGCCCTTTGGGCCAGGAACACTGCCTCCTGGGGTCCTCACAATCTCCCACCAACAGACACACAAGACTGGGCATCC
AGGGAGGGGGGAGTGGGCTCTGGGGCCCACAGAGTGAGAGGATATATGATGCCTCATTATGAGAGACAGGGAGGGGAGGAAAATGGGAGAGAGCCCGCCACTG
CCTAAAACACCCCCTCCTCTCAGACCAAAACCAGATGGAGGACGAACCACCTATATATTGAAACAGCTGTGGCTCTCATATTATTTGTTGCCACTGAAAAGAG
CGTGCTNTGGGGGGATTTCCTTTTTTCCCCCACGCCCTTATTGGTCCACTGCCGGGGGTAAGGCATTCATCTCCTTCGGCAGGGTTACTTATATATTTTTTTA
TTGGGGGGCCAGCAAGGTGGTTGCGCCGCGCCGGCCTCCACCTCCCGACATTTATTGTATGAAAATGATAAGAAAATAAAAAAAAAAAAAAAACACGA
>‘991115a2-040.scf” came from CONTIG 31 at offset 0;
“E:\SEQUENCE\export\EST_db\991115a2\991115a2-040.scf’ (77>650)
GAAAAATCCCCAAACCCCCCTAAAACTGTAATCTTATGGGTGATACAGTAGCAGGACTTAGAGGAGCTTGTCTCAGTTAGAATAACCTGTGTTCTCTATAGTA
TACTGCTATAAGCTGTTGTTGAGTTACTAAGTTGTGTCTGACTCTGCAACCCCCGGGGACTGCAGCATGCCAGCCTTCCCTGTATTCACTGTCTCTGGGAGGC
TGCTCAAACTCATGTTCATCCAACTATCCCATCCTCTGTAGGATATATTATAGAATTATATATATATATCACATTGAAAAGTTTCAATATGTAGGTATTTAAA
CTATATTAGTACTTATATATATATTATATAACCTTACTAAAATTCTGTAGATTTTTAAGATACTTTTAAGAATATAACTTGACTGAGATTTGAAGTTTGCGGG
GGGCTCTTTGGAGATTACANGTACATTATATTTTAGTGAGAAGACATAAAAAAGCCTGGTCCCAATTTCCAACTGTTACAAGGGATTTCCTTTTATTTTTTAC
TATACAACCTAAAGAAGTGCTGGATCTTGGAGGAGGCCAAAGGG
>‘991115a2-041.scf” came from CONTIG 32 at offset 0;
“E:\SEQUENCE\export\EST_db\991115a2\991115a2-041.scf’ (75>587)
CCGCTGGGAGCGCAGAGCGGCTGTGGGACGCGGAACTGCAAGAGTGCCGCCTGGCCTCGCTCCTTCCGTGTTTGCTTCTCTGCGGAGATGCCCAGCCGGGTGA
GCGACCCGAGGACGGCGCTGGGCCCGTCCCTGCTGAGAGCTCTGAAATGCACAAGGAAGAGCTCAGCAGCAAGATTAAAGAACAAAAAGTTGTTGTGGATGAA
CTTTCTAACCTGAAGAATACAGGAAAGTGTATAGGCAGCAACAGAACAGCAACATATTCTTTCTTGCAGACCGACAGAAAGCTTTCTGAAAGCAAAAACATTG
ATGAGCTGAGGAAGAATATAAGAATATAAACTCANAGAGAACAAATCAATAAAAGCCAACTGATTTACATAGATGTGTGCATTGTGGCAGTAGCTTTCTGTGA
GATTTACAGAATTGAAGAGATTACTGGTATCTTCGAGGGCCGATGTAGAAAAGTGTAGTTATGTACTACAGTTATTTTGCCTGCCTACTATAGTGTGCT
>‘991115a2-044.scf” came from CONTIG 33 at offset 0;
“E:\SEQUENCE\export\EST_db\991115a2\991115a2-044.scf’ (82>635)
GCACGAGGCAGAAGCTTCCCGCAGAGCGGGGCTTGCAGCTTCTTCACAAAATAATTGATGGCATTTGTGGCCGAACTTATCCTCTCTACCAAGATTATCACAG
TGTTTGGGATTCAACAGAATGGATGCATGTTTTAGAAGACATTACCACCTTTTTCAAAGCTGTAGTTGGTAAAAATTTATCTGATGAAGAGATATCTCAACAG
TTGAATCAGGTGAATTCATCTCATCAAGAAGCTATCATGAAATGCTTAAAAAGTAGGAAAGATGAAATCAAGAAGACTCTGTTGGGAGAAAATAGTGATATTT
CCTCTGCACGACTACGGGATTTTGATTGTCAGATAAAGCTTGCCTTTCTATGACAGATTGCTTCATTACAATGCACTTTTAACCTTTTCTGATGTAAAAAAAA
TGGGAAGGAAGCCTATCTGTTGAAGAGAAAGAGAACGCACAGCTATTAATCCTGGAGCGCTATAGGGGGCTGCGCTAAAAACGAAAGAGATACACATTCAATF
ECACGGCACGAGCCAGAAGAACAGGTCCGAAACGATGCCA
>‘991115a2-046.scf” came from CONTIG 34 at offset 0;
“E:\SEQUENCE\export\EST_db\991115a2\991115a2-046.scf’ (83>697)
GCACGAGGCTTTTGAAGATTCTTCGTTGTCAAGCCGCCAAATGGAGAGTGCGATCGCAGAAGGGGTGCTTCTCGTTTCAGTGCTTCTTCGGGCGGAGGAGGAA
GTAGGGGTGCACCTCAGCACTATCCCAAGACTGCTGGCAACAGCGAGGTCCTGGGGAAAACCCCAGGGCAAAACGCTCAGAAATGGATTCCTGCACGAAGCAC
TAGACGAGATGACAACTCCGCAGCAAACAACTCCGCAAGTGAAAAAGAACGACATGATGCAATCTTCAGGAAAGTAAGAGCATATTAAACAAGCTTACTCCTG
AGAAGTTTGACAAGCTATGCCTGAGCTCCTCATGTGGGTGTAGAGTCTAAACTCATCTTAAGGGGGCATACTGCTGATTGGGGACAAGCCCTAAAGAGCAAAG
ATAGCTACTTATGCTAACTATGCTGGATGGCAGAGAGCACAAACTTTGAGGCGAAAACAAAGGCAACAGAAAAAACAAACCACATCAACCCCCTAATCCAATA
CAAAAATTAAAACCAACAAAGTGAGCTATAAAAGGAAAACCCCCCCTGAGAGAGAAGAACATGCAAAAAAAGGGGAAAAAACTGGAATGAAGTGGCTTTC
>‘991115a2-048.scf” came from CONTIG 35 at offset 0;
“E:\SEQUENCE\export\EST_db\991115a2\991115a2-048.scf’ (77>568)
TGTGTCGTCTTGGAGGTGACTCGGCGTGATTGAATTTGCGGCATCTTCGCATTCACTCACAGGTCAAAATGCAGATCTTCGTGAAAACCCTGACCGGCAAGAC
CATCACCCTGGAGGTGGAGCCCAGTGACACCATCGAGAACGTGAAGGCCAAGATCCAGGATAAGGAAGGCATTCCCCCTGACCAGCAGAGGCTCATCTTTGCC
GGCAAGCAGCTGGAAGATGCCGCACTCTTTCTGATTACAACATCCAAAAAGAGTCGACCCTGCACCTGGTCCTCCGTCTGAGGGTGGGATGCAGATTTTTNGT
GAGACCCTGACCGGCAAGACCATGACCCTGGAGTGAGCCCAGNGACACCATCGAAACGAGAAAGCCAAGATCCANGATAAGAAGCATTCCCCCCGACCACAGA
GCTCATCTTGCCGGCAGCAGCTGGAGATGACGCCTCTTTCTGATACACATACAAAAGATCGACCTGCCCTGCTCTCCGCT
>‘991115a2-049.scf” came from CONTIG 36 at offset 0;
“E:\SEQUENCE\export\EST_db\991115a2\991115a2-049.scf’ (77>656)
CAGCTGTGCATCGACATGTTCTCAGTGTCCTGGGTAAGACCAAAGAAGCTGCCAAGATCCTCTCCAATAATCCCAGCAAGGGACTGGCCATGGGGATTGCCAA
AGCCTGGGAGCTCTACGGCTCAGCCAATGCTCAGGTGCTACTGATTGCTCAAGAGAAGGAAAGGAACATATTTGACCAGCGTGCCATAGAGAATGAGGTACTG
GCCAGGAATATCCATGTAATCCGACGAAGGTTCGAAGATGTCTCTGAAAAGGGGTCTCTAGACCAAGACCGAAGACTATTTATGGACGGCCAAGAGATTGCTG
TGGTTTACTTCCGGGATGGCTACATNGCCAGCCATTACAGCCTACAGAACTGGGAAGCACGCCTGCTCCGGGAGAGTCATGTGCTGTCAAGGCCCCGATTTGC
CCCCAACTGGCCGGGACAAAAAGNGCAAGCAGACTGACAGATGGGCGGCTGGAACCTTCTCCCAGCACCTGAGCTGGGCCCCTCCCGCCACCTTGTGCCTTAT
ACTAACTGGTGAGAAGGACAGCTTCACCAGCCTGTGTCCATGTTGGTTAAGCTAAAAGGGGAGGA
>‘991115a2-051.scf” came from CONTIG 37 at offset 0;
“E:\SEQUENCE\export\EST_db\991115a2\991115a2-051.scf” (83>429)
GCACGGGGTCGCCTCTCTGAGTTATGCAGTTCCATCCTTGTCGCTGCGGCGACACCCGCATTCTCCGTCGCCATGACTGAACAGATGACCCTTCGTGGCACCC
TCAAGGGCCACAACGGCTGGGTGACCCAGATCGCTACCACTCCCCAGTTCCCGGACATGATATTGTCCGCCTCTCGAGATAAGAGCATCATTATGTGGAAGCT
GACCAGAGATGAGACCAACTATGGTATCCCACAGCGTGCTCTTCGGNGTCACTCCCACTTGTTAGTGATGTGGTCATTTCCTCAGATGGCCAATTTGCCCTCT
CAAGCTCCTGGGATGGAACCCTTCGCCTTTGGGATCT
>‘991115a2-052.scf” came from CONTIG 38 at offset 0;
“E:\SEQUENCE\export\EST_db\991115a2\991115a2-052.scf’ (77>560)
TGGTGCTGCTGGACGGACTGGGTCCCCCTGGACCCTCTGGTATCTCTGGCCCCCCTGGCCCCCCTGGTCCTGCTGGTAAAGAAGGGCTTCGTGGGCCTCGTGG
TGACCAAGGTCCAGTTGGTCGAAGTGGAGAGACAGGTGCCTCTGGCCCTCCTGGCTTTGTTGGTGAGAAGGGTCCCTCTGGAGAGCCTGGTACTGCTGGGCCT
CCTGGAACCCCAGGTCCACAAGGCCTTCTTGGTGCTCCTGGTTTTCTGGGTCTCCCNAGCTCTATAGGTGAGCGTGGTCTACCAAGTGTCGCTGGATCTGTGG
GTGAACCTGGCCCCCTCGGCATCCGCAGCCCACGTGGGGCCCGCGGTCCCCGTGGTAAGTTCGGTAATCTGGCGTCATGGTGCTCCTGGTGAAGCGGNCGTGA
CGCAACCTGGGATGACGGCCCCCAGCCGCGATGGCAACCCGACACAAGGGAGCGGGNTACCCGCACGCAGTC
>‘991115a2-058.scf” came from CONTIG 40 at offset 0;
“E:\SEQUENCE\export\EST_db\991115a2\991115a2-058.scf’ (68>547)
CGGTGCTGCCTTGAATATTCTGGCACGATGGAGCAAGTTCGGTGAGCGCCGGCGGCGCCCAAATTTTAAGAAACATATCTAGGTCCGCGGTGGCTTGCATGTC
TPCTGGTGGGTGCTTAGCCCGGAGGGCCGGGAATCCAGATATGGGGCCGCCGGGTCTTCAGATACTGAATGAAGAATGCTGAAACTATGTGAAACATGAAACA
CCGTATCACGATCTTTTTTCATCTTGCTTTTTATATTGTATAACGTTCCGCCGAAAGAAAGGTGGAATGGATATCAGGGTCATAGTCAATATCCTTGTTCCAC
ACACCTTTTACCTCGCTCACCCTTTTATGTTTTAAATATCCCACCTCGATCAACCACTAACTCTTCTTAGGTTTCTTCAAAAAATCTATTCCATATCCGTTCA
GGGGTCCGTGTACTACTTTCAGTATTFCAATTTTAGGTCCAAACTCCTACACAAGGCCCCCATCAATA
>‘991115a2-059.scf” came from CONTIG 41 at offset 0;
“E:\SEQUENCE\export\EST_db\991115a2\991115a2-059.scf’ (83>633)
GCACGAGGATTATAACTAGGCTCAAACATATCTTTATTAATCAAAGGAGGAATCATTATAATCAACACATTTTCCCAACAAGAAATAAGTTTATTTATTCCTA
TAGCATAAAAACCCATGCTTTGGAATTCAGAGAACTCTTANAAAGCATTTTCTGCCTCCTGCTGGGTGTGGAAGCATTTTCTCTGCAAGAAGTTGTCAGGATG
CTTGAAGAAGGGGGAGNTGGTNTGGGCAGAGGNCAGGGGAATATGGNGGATGAGGGCAAACTTTGTAGCCCTATCATTCAACTTTTGAAGTATTAATTGTGCA
TGGGTGGTCAGACGTTGGCATGGAAAAGAAATGGCCCCTTCGTTCAGGAATTCAGCTGTAGGCATTGCAGTTTTGATGCACTCATTGATTTGCTGACATACTC
TCCATGTAAGGTTTATCAGATTAAAAAACATAAGAATAGACAGACACAAACACCAAAAGACGNGAGTTTTTTGTCAGNTGGTTTGGAGGGTTGGGTTTTTTCT
TACCACGACGCGTTGTGTGTTTTAAACATTTTGC
>‘991115a2-060.scf” came from CONTIG 42 at offset 0;
“E:\SEQUENCE\export\EST_db\991115a2\991115a2-060.scf’ (82>480)
GCACGAGGTGATGGTGCAATACTCTTAGCTGCCATAGAACTGAATTGTGGTGCAGCTTGGGAGTGGGCAGTCTATGCATACGACCCAAGGCCTGCAAGCAGTT
GGTTTGGGAGCTGACTTTGATTCAAGTGGGTGAGAAAGAACACAGGCTTCATCTCTGCCCTCACCTTCTGCTCGCCAAGAGCAGCCCTCAGCGTFTAGAACCG
TTACAGAGGTATTAAAACATGCAGATGGCTACCTACGCCCTGTAGTCCTATAAACTGGGTTCTGTTGAGGTCACTCTGTGCCTTAAAGTTAGTCAGTTGGGAC
CTAATCTAAAAAAAAATAGATAGTTTTTTTTTAANAGAAAGATTTTGGTGCTTTTGTTACTAGAGACCGATTCTCCATTTTTTGGTGGGGG
>‘991115a2-062.scf” came from CONTIG 43 at offset 0;
“E:\SEQUENCE\export\EST_db\991115a2\991115a2-062.scf’ (83>591)
GCACGAGGCTGGACAGCTGCATCCCCACGCTGGGCCTTCCCCCCTGGGAATCCAGGTGCTGGGAGCCTTCCCTGGGGTTCCTTGCTCCCCTGGGCAACTTGTA
GAATGATGATGGGCCTGTGACACAACCACACATTGCAGGTGCTGTCACCTTTAAATAGTGAGTTCTTTTGATTTTTAAACATTTTCCATAAATCCTGGACTAA
TTCAGATATCATTTTGATTAATTACATAAGAATGACATCTTTTTGACCTGAATGTGGTAAAAGGAGGTCCATGGGCTACAGGGGGTTAAAAATGGTTTTACAG
ATATCATTTACTCTTTTTGCATTTTATTATGAAAATTTGGATAGAAAAATGAAAGATAGAGCTCTTGTCCTGGACCACTGCCAGGATTGGTGATTTTGAAGCA
CCTCCTGCCCCAACCCCTTGCGCTGGGGAAGAGAAATTAACACAAAGAAGATTTAAATCAGAACCCANAGGAGAAAGCCGATGGGAAAGAAAAGCGGC
>‘991115a2-066.scf” came from CONTIG 44 at offset 0;
“E:\SEQUENCE\export\EST_db\991115a2\991115a2-066.scf’ (76>490)
CATGGGAGTTGACATCCGCCACAACAAGGACCGAAAGGTTCGACGCAAGGAGCCCAAGAGCCAGGACATTACCTGAGGCTGTTGGTCAAGCTGTATAGGTTCC
TGGCCAGACGAACCAACTCCACCTTCAATCAGGTTGTCCTCAAGAGATTGTTCATGAGCCGCACCAACAGGCCACCGCTCTCTTTTCCCGGATGATCCGGAAG
ATGAAGCTTCCTGGCCGGGAGGGCAAAACAGCTGTGGTCGTGGGGACTATAACCGATGATGTTCGTGTCCAGGGAGTGCCCCAACTGAAGTGTGTGCTTTGCG
AGTGAGCAGCCGCGCCCGGAGTCGCATCCTCAGGCCGGGGCCAGATCCTCACCTTCGACCAGCTGGCCCTGACTCCCCCAGGCTGGGCACTGTCTCTCTCTGT
CG
>‘991115a2-068.scf” came from CONTIG 45 at offset 0;
“E:\SEQUENCE\export\EST_db\991115a2\991115a2-068.scf’ (83>551)
GCACGAGGGTTTTTTTTTTTTTTTTTTTCATTTTGTTTTCCATATATTAAAAAGCAACATAGTACAAATGCTGTATTATTAAAAAGGACTCAAATTTTACAAG
GAATAATTTTTATAAACAAGTTCTACAATGAAAGGGAAACAGCATCTTGATACATAAAAGTGTTGTTAAACAAGATTTAAAAATTTTTAAATCAGAAAATGTT
TTAGACTAAATTCTTTCAAACAAAAACAAAAAGCAACCTGTACAATCTCCATAATAAAATGGGTCCCTGTACACTTGGCCTGGATACAATGTTAAAATTTTCA
AAGTTTGAGTCAGAGAGGCAGGACTTTAAAAAGACATTTTTAGTAAAACATTTTACCATTTTATCCAACTATGCATTTATTTTTTGTTTTTCAGAAAAGGGGA
TTATATTAACACTGTTTTTTACTGTAAAAAAAACTTTAGGAAATTTACTTCCCGAG
>‘991115a2-095.scf” came from CONTIG 45 at offset 0;
“E:\SEQUENCE\export\EST_db\991115a2\991115a2-095.scf’ (83>123)
GCACGAGGGTTTTTTTTTTTTTTTTTTTTTTTTTTCTTGCA
>‘991115a2-070.scf” came from CONTIG 46 at offset 0;
“E:\SEQUENCE\export\EST_db\991115a2\991115a2-070.scf’ (84>573)
GCACGAGGGGAGGCCCAGGCCTGCTGGACTTGGGAGTTTCCAGTGTTTCCCCTAGCGCCTGCGTTGGGGGCGGGCTTAAAGACATTGTAATTAGTTGTCGACA
TGTTGCGCTTGGTGAGCTGGAACATCAATGGGATACGGAGCCCCTTGCAAGGGGTGAGATGCGAGGAGGCCAGCAGCTGCAGCGCCATGGCTATGGGGCGCAT
TTTGGACAAGCTGGATGCAGACATCGTCTGTCTTCAGGAGACCAAAGTGACCAGGGATGTGCTGACAGAGCCCCTGGCTATCATTGAGGGCTACAACTCCTAT
TTCAGCTTCAGCCGTAACCGCAGTGGCTATTCTGGTGTAGCCACTTTTTGTAAGGACAGTGCTACCCCAGTGGCTGCTGAAGAAGCCTGAGTGGCCTGCTTTC
CATCATATGGGGAGGGGGTGTTATGGAAAATGAAGACTTACCAAGAGAACTTGAGCTTGATAGGAGGGCGGCCCTCTA
>‘991115a2-071.scf” came from CONTIG 47 at offset 0;
“E:\SEQUENCE\export\EST_db\991115a2\991115a2-071.scf’ (77>427)
GGGGCTACAGGCTCGAGGATGGCGAGTGTACAGATGTGGACGAGTGTGAGCTGGGCACGCACAACTGCCAGCGGGCGCCGTGTGCCACAACACCAAGGGCTCC
TTTTACTGCCAGGCTCGGCAGCGCTGCCTGGAAGGCTTCCTGCAGGACCCCGAGGGCAACTGCGTGGACATCAATGAGTGCACATCTCTGTCCGAGCGGTGTC
GGCCCGGCTTCAGCTGCATCAACACGGTGGGCTCCTACACGTGCCAGCGGAATCCGGGGATCTGTGGGCGCGGCTACCACGCCAGCCAGGACGGGACCAAGTG
TGTGGACGTGAACGAGTGGAGACGGGTGTGCACCGCTGNCGC
>‘991115a2-072.scf” came from CONTIG 48 at offset 0;
“E:\SEQUENCE\export\EST_db\991115a2\991115a2-072.scf’ (84>559)
GCACGAGGGAGTGTCTTTATCGCTGTGGAATCTCCATGCAGGTTCTCGGGGTGCCGGGCGAGTCTATTTCCGGGCCCGCGCTCGGCCCCGGCCCGGCGACCTG
TTCCAGCCTCTGCCCGGGGTCTGCGGGGCGGGAACGCCATGCCGTGGGCTCTATTCCGAGGCCGAATCTGGTAGCCCAAAGATCAAGAAACCTACTTTTATGG
ATGAGGAAGTCCCAAGCATACTCATCAAGATGACAGGCTTGGATTTACTGAAGATTTTTTAGCCAGCAGTACAAGAAACGAAACCACCAACTACAAGCTATGA
CCCAGCACAGTGGAAAGAAGCTACAGACAGCGATTGAGCAGCTAAGTNAGATTAAAAATGCACCAAGTTGGAAGACGACACCATAATGATGTGTAGTGAGATA
GATTTGGAGGACCGAACAGGAAATGTGTTATGATATATGACGATACAATGGAGGTTTATGTGGA
>‘991115a2-074.scf” came from CONTIG 49 at offset 0;
“E:\SEQUENCE\export\EST_db\991115a2\991115a2-074.scf’ (83>452)
GCACGAGGGCCATCTTTGCATTGTTCCCGTCTCCGGCTTCCCTGATCGCCGCAGCCACCTCCGCCGCGCGCCATCTCCGCCGCCGCGGGCTCCGGCAGCTTTA
CCGCCAGAGTCCTCGAACTCCCGCTTTTCTTCTCAGTCCTTTGCATCGGATCACCGGAGTGCCCCATCATGTCAGACGCGGCCGTGGACACGAGCTCCGAGAT
CACCACCAAGGACTTAAAGGAGAAGAAGGAAAGTGTGGAGGAGGCGGAGAATGGGAGAGAGGCACCTGCAAATGGGAATGCTAATGAGGAAAATGGGGAGCAG
GAGGCAGACAATGAGGTAGACGAAGAAAAAGAGAAGTGGTGAGTAGAGAGAGAGGATAAAG
>‘991115a2-075.scf” came from CONTIG 50 at offset 0;
“E:\SEQUENCE\export\EST_db\991115a2\991115a2-075.scf’ (77>568)
GGACGTTCGGCTTGTGAGCGGGAACTTCTCGGTCGCGCACAGAGAAAGAACGATTTAAAAATGGGTGATGTTGAGAAGGGCAAGAAGATTTTTGTTCAGAAGT
GTGCCCAGTGCCATACTGTGGAAAAGGGAGGCAAGCACAAGACTGGGCCAAACCTCCATGGTCTGTTTGGACGAAAGACAGGTCAGGCTCCTGGATTCTCTTA
CACAGATGCCAATAAGAACAAAGGTATCACCTGGGGAGAGGAGACGCTGATGGAGTACTTGGAGAATCCCAAGAAGTACATCCCTGGGACAAAGATGATCTTT
GCTGGCTTTAGAGAAAGGAGAGAGGAAGACTGATAGCTTTCTCAAAAAATACCAAGAGTATAGGTGGCCCTGCCTATTTTTTATGATANAANGTCTATGATTT
TATGTGTACATATTTATTGACTATATATATATCANATATGACGCTGAGGATATTTTTTGACGCCTGTTTAATAAATGTTG
>‘991115a2-076.scf” came from CONTIG 51 at offset 0;
“E:\SEQUENCE\export\EST_db\991115a2\991115a2-076.scf’ (84>556)
GCACGAGGAAGAATCTCTCTTTAGTTGTTTGTACGGAGGTGAAAAAAACAAAAAGATCCTTCAAAAATGGCAATGGTATCTGTATTCCTCAAGCAGGCCTGGT
TTATTGAAAATGAAGAGCAGGAATTCATTAAAAGGGGTAAGGATCCAAAGGGGGTCCCTGGGTCAGCAGTGAGCCCCTATCCACGTTCAATCCATCCTCGGGT
GTTGAGGCCTTGCACAAAGCAATCACCGTTAAGGGGGGGGGAGAAGCAACCATCTTTGAAATTCTGACTTAAAAAAACAATGCCCAACGTCAGCAGAACAAAG
CGGCCTATCTGCAGAAGAAAGAAAGCCCCCGGAGGAATTTTGAAAAAGACCCCCTCGGCACCTTGGGGAGTTGTTGGGGTTTTTGAAAACCCGCCCAGTTGTT
GCCAAAACCCCGGCCGCCTGAGGGCCTGGGACGAGAAAACCTCTGATGAAATTGGGCTCCA
>‘991115a2-077.scf” came from CONTIG 52 at offset 0;
“E:\SEQUENCE\export\EST_db\991115a2\991115a2-077.scf’ (78>624)
GCCGATTTAACTTTACAGTAACAGAATAGACAGCACAACACAGACTCTCTCAATACAGATAAACTCACACATACTGGAGATTATATATAATAGATATATATAA
TTATTGCATTGTAGTGTAATATTTATGCCTACTATACTGTATAACACGTTATTCGGGATATGCCATTTCTGAGACACGATAACAAAAAAAAAGTTGAGGAAAT
TATTTTGCTTCTATTTATAGCTCTGTCAGTCAGAAGACTCTAAATGCTTTGCAAAAAGGGGTTCACATTTGCTTAAATGCTTCATCACAGTCACATTTATAGT
GACTCTAAACAAAGAGAAGCAGCACTGTCATCAGAGGCATGATAAACCAAATATGANATGGGAATGTTTAATTACCTAGTATTGGTGGGGTAGTACTGGTGGA
TTTATTGTGATTGTTTTGTTTTTCAATTACTGCTTTGCCTATAAGCCYTCAATTTAAAAAAAAGAGTGCTTCGTTTTTAGATGAATCTCCAAGGATCCTTTTG
TGTTGGTG
>‘991115a2-078.scf” came from CONTIG 53 at offset 0;
“E:\SEQUENCE\export\EST_db\991115a2\991115a2-078.scf’ (78>494)
CTGGTGTTGGAGCCGAAAAAGCTGGTGGTTTTGCCCCATATTATGGAGATGAACCGATAGATTTCAAAATCAACACCGATGAGATTATGACCTCACTCAAATC
AGTCAATGGACAAATAGAAAGCCTCATTAGTCCTGATGGTTCCCGTAAAAACCCTGCACGGAACTGCAGGGACCTGAAATTCTGCCATCCTGAACTCCAGAGT
GGAGATTATTGGGTTGATCCTAACCAAGGTTGGGGATGCTATTAAAGTCTACTGTAACATGGAAACTGGGGAAACGTGCCTAAGTGCCAGTCCTTTGACTATC
CCACAAAAAACTGNGGGACAGATTTTGGTGCTGAGAAGAAAAGTTTGGTTTGGAGAATCCTGGGGGGTGGGTTTAGTTTAGTTTGGCATCCTGAACTTCCGAA
>‘991115a2-079.scf” came from CONTIG 54 at offset 0;
“E:\SEQUENCE\export\EST_db\991115a2\991115a2-079.scf’ (84>272)
GCACGAGGCGAGTTTTTTTTTTTTTTTTTTTTAGTATAGAGGTCCTAGAATAATTTTTGGGTTTAGGGTTAGGAGTAGTAGGGGTAGGATGTGTAATGATATG
AGTGCCTTTTCCCGTGTAAAGGATGGCGAGATACTATTAATGTGGTAGGGATATTTTTCTTGTTGGGTTTTAATTACCATGCCTA
>‘991115a2-080.scf” came from CONTIG 55 at offset 0;
“E:\SEQUENCE\export\EST_db\991115a2\991115a2-080.scf’ (77>649)
CTCACCATCAACCCCCAAAGCTGAAGTTCTATTTAAACTATTCCCTGAACACTATTAATATAGTTCCATAATACAAAGAGCCTTATCAGTATTAAATTTATCA
AAAATCCCAATAACTCAACACAGAATTTGCACCCTAACCAAATATTACAAACACCACTAGCTAACATAACACGCCCATACACAGACCACAGAATGAATTACCT
AACCAAATATTACAAACACCACTAGCTAACATAACACGCCCATACACAGACCACAGAATGAATACATATGCCCCATGCATATAAGCAAGTACATGACCTCTAT
AGCAGTACATAATACATATAATTATTGACTGTACATAGTACATTATGTCAAATTCATTCTTGATAGTATATCTATTATATATTCCTTACATTAGATCACGAGC
TTATTACATGCCGCGNGAAACCCAGCACCGCTAGCAGGGATCCCTCTCTCGCTCGGGCCCATAACGGGGGGGCGCTTCTATGATTTACAGCATTGGTCTTCTT
AGCCCTCTCTCTAGGCCTTTTTCTCTAATAGAAATTGAGACTAGGTAATAGCTT
>‘991115a2-082.scf” came from CONTIG 56 at offset 0;
“E:\SEQUENCE\export\EST_db\991115a2\991115a2-082.scf’ (82>671)
GCACGAGGGATGAGTGCTAAGGGTTGATGCTGGTNGCCCCTGGGAGCCCCCGGTAAGCCAGGGCTGTCCCCTTGGCGCTTCATAGGAATGTCGCTGGTGATTA
CGAGGTGCAGCGTGTGTCTTCCAGGACCCTAAAGGGTGACAGAGGGGGATGCCTGGTCCCCAAAGGGTGCTGGATGGGTGGCTCNNCTGGCAAAGATTGGCGT
CCGTGGTTCTGGACTGGGTCCCCATTCGGTCCCTCCTGGCGCCCCCGCTGGTGCCCCCTGGTGGACAAGGGTGGAAGACTGGTTCCAAAACGGGCCCCAACCG
GTCCCCACTGGTAGGCTCGTGGTGGCCCCCGGTTGACCCGTGGTTGAGCCCTGGTCCCCCCCGGCCGCTGGTGGCTTTCGCTTGGCGCCCCCTGGTGCTTGAT
GGCCAACCCTGGTGCTTAAAGGCGAACCTGGTGATGCTGGTGCTAAAAGAGACGCTTGTCCCCCCCGGCCCTGCTGGGCCCGCTTGACCCCCCGCCCCCATTG
TTAAGTTGGTGCTTCCGGACCAAAGGGCTTGTGGCAGCGCTGTCCCCCTGTGCTTATGTTTTCCAGGGCTGTTGA
>‘991115a2-086.scf” came from CONTIG 57 at offset 0;
“E:\SEQUENCE\export\EST_db\991115a2\991115a2-086.scf’ (76>860)
TATCGTACGGACAGAGGGThTATATCACCTTATTCCATATCCCCTCTTTGGGCTGTGGTTGATGCAGTATCATGGATGGGGGATGGACGCCCCCAAGGTTCAT
TTGCCCGGTGCATCTTACCTTGGGACAGACTCCGGAACCAGTTGGGGAGGAAGTGTCGGTCTTTGGAGAAAGCGGAGAGGATGTTGACGTGGTTGCCTCTATA
TAAAAATGGGGTTATCAACCCAGGTGGGTGGGCCCTTTCAATAATATAAAAGCTCAATGTGCTACGGCCGGCATTTTTCCATTACTCCTGTCCCCCCCCAGTC
ACTCTGGGCTTGTCTTAGACCTTATGATGTGTCCATTTGTTTTTCTTGTTGCTTGGGAGGGCCCAATACTTTTTTTGTCCATTATATTCCTAAGGGATCCTCT
TGTGGAGGTACGGCTCAAATTGGGTTGGTTGCTCGTGTATAAGCAACAAAGGGCTGAAGGGCCAATTTTCTTTGGAGGGTTGGGGGCTGGTGTTTAACACCAA
AGTTGAAAGGGGGGCTTTCTGGTAACCCCAACGAGAATAAAAATGGGGTCCCCTTTCGCACCTTTGTTTTCTTTGGCCCGGCCCAAGACTAAAAACAATTTGG
CGAGCCACAGGTCACCACCCGGGCCCGCACCTTCGTTTAGGGGGGGAACACTCCTCCTATACTCCCTCATATATTGGTAACACATTTTCTGGGCTCTTGTTGT
CTTCGTCTTGCTTTGTGGGTTTGCGGGGGGGGCCCCCCTCCACTTTCTTGTCTCA
>‘991115a2-087.scf” came from CONTIG 58 at offset 0;
“E:\SEQUENCE\export\EST_db\991115a2\991115a2-087.scf’ (83>749)
GCACGAGGCTACANTCGGGCCTTGCAAATACCATCCCCCCCTNGCCTGGACTCCGCAGCTGACTGTATATTCCCCTTGCNGCCATGCGGGGACTGGGCTTATA
GATACGTCCTGGGTCAACGCTGTTACGAGGAGGGGACGGAGGGACAAACAACCTCCCTGGACCGAAGAGCAGAGCCTGCGAAGTGAAAAAAGATCCCACGAGA
ATGAAGAAGACGCGCTGGAGGCTGGCGGACCATTCCTGTGGAAACTGCCTGGCCCCGGGACTTCGAGAAGAAACTACAACATGTTTACATCTTCCCTGTGCAC
TGGCAGGTCGGGGCAGCTGGATCAGCACCCCATTGACGGGTACCTGTCTCACACCGAGCTGGCCCCACTGCGCGCCCCCCTTATCCCCATGAAACACTGCACC
CACCCGTTTTTGAGAATGTGACCGGACACGACAGACATCGCCTGGACGAGGGGGCGGCTGCTGGCATCAAGAAAAGACATGACAGGACCCGGATCCAGCCTCC
TCGAAAAACAATTTTTTTTTGACTCCCTCTGTTCCCAAGTTAAAGTGGAGGTGGTGGGTTGCTGGGAAGGGTAAATAAAAAAAAAATTAAGGGTAAAAAAAAT
TAACAAACAAATTATAATTAATAGGTGTCCATAAGGCCG
>‘991115a2-091.scf” came from CONTIG 59 at offset 0;
“E:\SEQUENCE\export\EST_db\991115a2\991115a2-091.scf’ (78>508)
CGCCGGCGTGCCCACGTCCAAAGGAAAGTCCAGGAGGAAAAAGGATTTACGAATCTCCTGCATGTCCAAGCCGCCGGCGCCCAGCCCCACGCTCCCCCGGAAC
CTGGACTCCCGGGCATTCATCACCATTGGAGACAGGAACTTTGAGGTGGAGGCGGATGACCTGGTGACCATTTCAGAGCTGGGCCGAGGTGCCTATGGGGTGG
TGGAGAAGGTGCGGCATGCCCAGAGTGGCACCATCATGGCCGTGAAGCGCATCCGGGCCACCGTGAACTCTCAGAGCAGAAGCGCCTGCTCATGGACCTAGAT
GTCAACATGCGCACGGTGGACTGTTTCTACACCGTCACCTTCTATGGGGCCCTCTTTAAAGGGAGACGGGGGGATTTGGATGGAGCTTATGAACCGTCCTGGA
CAGTTCTATGGAGGTGCTT
>‘991115a2-092.scf” came from CONTIG 60 at offset 0;
“E:\SEQUENCE\export\EST_db\991115a2\991115a2-092.scf’ (84>529)
GCACGAGGGCACTTTATGATACTTTTTCTGCTTTTGGGAACATTCTGTCCTGCAGGTGGTGTGTGATGAGAACGGCTCTAAGGGTTATGCCTTTGTCCACTTC
GAGACCCAGGAGGCTGCCGACAAGGCCATCGAGAAGATGAACGGCATGCTCCTCAATGACCGCAAAGTGTTTGTGGGCAGATTCAAGTCTCGAAAAGAGCGGG
AAGCCGAACTTGGAGCCAAAGCCAAGGAATTCACCCATGTTTACATCAAAAACTTTGGGGAAGAGGTTGATGATGAGAATCTGAAAGAGCTATTTAGCCCAGT
TGGTAAGACCCTAGTGTTAAGGTGAAGAGAGATCCCATGGGGAAACCAAGGCTTTGGTITTGTGAGTTACAAAAACACGAGATGCCAAAAGGCTGGGGAAGAA
TAAAGGAAAAAAATACTGGAAGGCATTTCGTGCCG
>‘991115a2-094.scf” came from CONTIG 61 at offset 0;
“E:\SEQUENCE\export\EST_db\991115a2\991115a2-094.scf’ (72>264)
CTGCAGGCCTGTATGCTTGGGAGGGCATTGGGGGGGCCAGGAGNNGAGGAAGGGGGACGCACAGAAGGGATGGAGGATGGGTTGGGATGGGCATCCACCTGCA
CCTTCGCATGGNAACGTGGAGTTCCTGGAGTTGCAACCTCCTGGGGGAGTTTGGGTGGATAAGCACAAGGGGAAGGCCCTGGGCGGTGCC
>‘991115a2-096.scf” came from CONTIG 62 at offset 0;
“E:\SEQUENCE\export\EST_db\991115a2\991115a2-096.scf’ (78>582)
CGGCAGTAGCAGCCATGAAGGTCGAGCTGTGCAGTTTCAGCGGGTACAAGATCTACCCAGGACATGGGAGGCGCTACGCCCGGACCGACGGGAAGGTTTTCCA
GTTTCTTAACGCAAAATGTGAGTCGGCATTCCTTTCCAAGAGGAATCCTCGTCAGATCAACTGGACTGTCCTCTACAGAAGAAAACACAAAAAGGGACAGTCG
GAAGAAAATCAAAAAGAAAGAACTCGCCGGGCAGTCAAATTCCAAAAGGGCATAACTGGTGCTTCTTTGCTGATATATGGCCAGAAGAATCAAAACCTGAAGT
AGGAAGGTCAACGAGACCAGCTTTAGGGCTGCCAAGGAGCAAAAAGGTTTAGCAGCATTAAAAGACGCATGCTGTGGAAGCTCCACAAAGCAGCACTAACAAA
AATGGAACCGGGAGGTTITGTCTGGGTGTGAAAAGCTAGTGCGATGATTTTGAAAGAATGATTTAATAAAAAAAAAAAACGAGGGGGCCGGCCC

TABLE 3
GNCCCTTATAAAGTGNNNNTCCNAATANNNGGGGGTATTACTATTTACAANTNTTCNTCNCTATAACN
GAAGNCGCNATCNCAANCNGGCCCTTNANCNAATANCTNNTCTTCCNTANCANNCTANATTTCNTGAN
CCTTAATNCCGTACAANNACTNAATACNCTCTTGNTACATCCCACNNTNNTATNNNANTNTNNTCATT
ACCTCATNTTNCTCTAANNNGGTTNTTTGAAATCTAGCTAATTCCNNNTANGAGGNAGNCTGAANTTT
NNTNANANNTTAGACATCCGNTNTATANAGAANTNNANNNTANNCCTNNATGANGNNATNNNACTCN
GTTNNGTNNATTTAACGNTNNTNNNTATNCTTGNNNGCCANGATCCATATNNNNCNNNTGNNGNTCC
CCNGNAATTNANNCCGGNNGCNNGTNGAANACNCNGCNAGANATAAGGGGGACANNTNNCTGAGTN
NTCAATNACANGATNCGATANAACTTACTNCTNATAGTNGNNAGTTANNNGCAGTNATANCNTTACA
ANNAANNNNGGNNTNNNGNNTNNCTTATTANGAGGGGNAGNAATNNNNNAGNCTNAGGANTNACNN
CNNNTNACNTNNATATNANGCTNANNAANNNACNCNNGNANGGNCNNANANNGAGTTTTANTTAGTN
CANAGGTGNNNNNNTAAANGNNTATNATGNGGATNNGNCGAGNNATNGNANTNTACGNNNNNTTAG
ATNNGGGGNAAGGATNGNNNANTTGATTCTNAANGGNGGNNGAGGCANGNGTNANNNGTCNNGNNN
CTTCTNNCNNNNGANGNGGACAGAATNNNGNNGTGATAGGGNTTCNNGNTNGNTNNAGNNNNGAAN
ANCNNGNGNGGNNANNANAGTANNTNGNGANGNNTTCGNCNNANNGNGNAANTCNTAANTNGNNGN
ANNNANNNNCTNTNNTNNGTANGNGNNGAGCANAGANATCAGANGNANGCATNTACNNNCNAGGNG
GNNTGAGANGNANATANANGTTNACNAGNTNNNACNCACCNGAGNNTTTNGTANNCATTNACNNNGA
ANNTANNTNATNNANTGTNANTGANNCNTAGNGACNGTTAGANTANGANTAGTGTNTANGTNANACN
NAGNCNNGAGAGTGNANAGAGAGATATNNNATGNACNNGTAGAAATNTNTNNTCNCGNGATATGTAT
ACGANCTGCGCGAGACG
TTCANCGGCNTTNNNNNGNANNCNTNNNTNNNGGGGGTATTANTANTNCAAANCNACGCGCCTATAG
NGAGTCTNTANTGCANANGTGTTNTNGGANNGAGTTNTNNATTNCTNGCANNNTNCTCTNNNNCTTAT
TGNNAGNGTCNNTTCNNNATAACTNTTCNTNACCTGACGGGCTANTNANAGAATNGNAANTCGNNNT
NGTGNTNNTATNAGNGCACANANAGGNNNTGNGANACGGTNCNNGGCCNACNGGAAGACTGNTCCA
TTGTNNGTNCTAGCGGGANGNNNTNNTAGTNTTTTTNTGNCNNNAGNNTNANAGNGGGNGACNNCNN
GNGGNTCAGCTGGCTGATACTCGCGNANTCNTGCNNNCNCNGATNGANNGTNNNNAATNTCNCAANA
TAANGTCGANNTNTCNATAANGAGCGNCGNNCNNTAANCTGGNGCATCTNCNGTGNGNCNAAATTCG
GTANGTNNTATNTCTANNNNACGNNGTNNNGNCCNTANANTTCNATACATNANNTGANNNNGNCNGA
GGGANCNCGTTTNANGAACTANAAAGNGACGNGTGGATTANNGCCNCCNGTGTACTTGCNGAGTNGT
NNCNNANNCNNANGANNNGAGNNCCNTTGNNNNANATCNNNTATNTNGNNNNTTCNNACTGNCGA
TNNTNNNNTCNGNNANNNANAANTNNATGNNCGNNTGTTATGTNCNNNAGGANNATGNTNAGNNCN
GATGGTNNCNTGGGTGNANTCCGAGNANGATNTCNAANNGGCTTNGAGNGNNANATCGAACNTANTG
ACNNGACNAGANCCNGNNCNTANNCCTCNCNNAACANNTGAANNNNNGNGATGGNCGCNNAGANNN
TNNCAANTACNANNAGCTACGANNNGNGGTGTNNACNNTNNNGTNNNNCNNCNAGCTNATCTNNTCT
GNNNGNGNTCGTGACNNNTGANCTNNNCNNNAGCNTANCGANTGCNTGTATAATNAGNNNATATNGT
TNTTATNCNATGNCGNGTGCATCATGNANGAGNNNGTCATNNNTNNTNNCACTATGTCTACGACTTGA
NANCCNANNTCAGNNCANTNCGTATNTCCNNNGANGTCNCNNCGTNNGNTCTCGATGAAGNTCNNGA
CGNAAGTNNATGTNCCNNATTNCNGTATGACNNCATNGACNTTATCGTGNNANNNGCTNACNNTANA
TGACGNNCTTCTNTGNANTNCCGTGCNNGCGCNTCCTGNNNACCGACGTCCTANNNACGNCNCANGTA
TNGCATCAGGAGNACANGTTNCNNGTNNNTACNCGTGGTAAGNNNNGTNTCNTNNCTTCNNNNTTCN
CN
GCCCTATANGGAGNNGTATNNCAGGGGCGGCCGCTCTAGAACTAGTGGATCCCCCGGGCTGCAGGCN
GAGGTCTCCGGCCCTGTNTGCGCANNANGAGGCTGGGANATGGGCCCCAGGTCCCCCTGAGCCAACCT
NCCGAGGANGANCTTANANAGGTNNNACCAAGANGCTGGNGCACNANATGAACCATCNNNCCANTNT
ANNANTNCTTCANCCGCTGCGATNNNTANNNAAAACNNTANNTNNANNGNTTNTNNTATATTTANCC
CNTANAANNATNNNTATNTNTNANNNTNTAAAACNTNGNNTTNNNTAATNNTTNTTTGANANNNTTNT
TCTCCCCTNNAANNANNNNCGTTNTAAATNANACNTATNNNAGNGAGNNNAACATNAATNNNNTGC
NGNNTTTAANNTNTNNNNTNAACTTATTGCATAAATNGNNTTNATATATNANATNTATNCANNAAACA
NACNNNATTACNNNTATTTTATTNTNTTATANNNNNANTNANTCTNCNAATTNNTGTNAAANNTCGAN
TTCAAATTAATTATNNTNNTNTNGNNAGCNNATCTANNNGNNACNTNTTNTNANTTATGTTGNGANTN
NNNNANANCNCTGTGTACCCCTAACTNNNCNNACACCCNNCCNNCTNNCCTANTCTTCNNTTTCTATN
ACCGAATANNACTTCTNACNNNNCTNTTNACTNACCCNNNNCCNNANANANTTANNATCTTNAANTNC
NCCAANNANTCACNTNNGNTNANTTTNGCTTTAATATAANNTCGAACNNNNNCCTAAGTNAATTTN
NTCTNCTNTTNACCTAGNNTNTANNNAGAAANANANATNATNNTNNTNANTATANANNNTNANNAGA
CTAATATANATNAANCTACCATNNNNNNAAANANANANANNNTTCNNANCTTCNACAAANATATNTG
NNATNNNATCTANCTTATGTNCNNTAAGAANNNNNGNTACNATTNAAGNNTAAATNTCANANGNATC
NAACCNTNTCNATCGCCATAANAANNCNNATANNTATACANTAATCTCANTNANTNCGTANANTNNN
NNATATNNANNCNTNNGCNC
GNGCCTNGTGGGNNTTNTNNANNTATATTNANTTGNNNNNNNTNNANNTNCNCNTNCTTNNNTCTTNN
NGNNNCNTTCCCNTNTTCTTNNNNGGNNGNNTTTNGNAGNNTTNNATNNTTGAGCCNNNNNGATCG
TATTACAGTGGCGGCCGCTCTANAACTAGTGGATCCCCCGGNCTGCAGGCCGCTTCGNGATCTGNGGN
CCCCGGTGCAAACATGGCCAAGTCCAAGAACGNACNCCACGCACANNCAGTCCCGAAAATGGCNTAN
AAACGGCATCAATANACCCCGATCACAATGATATGAATCTNTTAAGGGGGTANACTCCANGTTCCTGA
GGAACATGCGCTTTGCCAAGAAGCACAACATAGAAGGNGCCTNNAANANGATGGNCGCNNNCCAATG
NCAAGGCCATGAGTTGCACCGTGCTGANGCTGTCAANGCCCTCCTCAAACCCAAGGANATCANNCCCA
AGATGCCAACAGGTGGNAANTCGTAAGCTCAACCNACTTGCCTACATTCCTTACCCCAAGCNGGNNAA
NCGCGCTCGTTGCTTCGNATCTGCCAAAGGGGCCTTNATGGCTCNTGNCCGGGTCGAAAAAGATNCGT
AGGGTCAANGGTCTTTNNAACCCATTGGNCCAANGCCACCTTGGCNNNCTTGGCTNNTTNACAAACTT
GGCCAAAGGGGTTGGTCNNNTGCGCCNCCAANCCAAAAAGGTTTNCCCGGNGANGNTAANAGNAACA
CTTTTATTTNTGCCCAANGTTGTGNGATNGACCAGNAAANNGNAACNTTGGGNNGTAGGAACCCCCCC
TTGGNGCCCTNGCNTTGNNCNCTGGCGATNGGGNGNNCNTGANTGNTNACCNCNCTTGGTGGCCTANT
TATTNCGNCCAAAAATTNANATATCTCGCGGCACCTGGGGTNCNTNNTNNNGNNTTCTNTTACAAGT
ANNTNCCCCTTTTGCGNNAGGTGGGGGGGGTGNCNCGCGTATANACCCTTNTNTTCTCGTCCCTTCTTT
ATATTGTGANGNCTGGNTNATANTTCTCAATACNTGNACNTCTNCTNTGTTGGNCTTGCNTNGGANTTC
NTNNNACANTAANTCTNCNNANNTAGTATTTTNCNAANAGAGTTACTCGATACTTACACCCNTNGAAA
GNTNNCNNGATTTANTGCNTTCTTNNTCTTTTTNNTNTCTCANNTACGTGNNGNTANCTTNTNNTCNGT
TGTCNCTCTCCCNTCNNCNGNNTTCTATGTANNNNNNNTNGTTNTTACGNTATNNTCTCATNACGTNAC
NTCTCTCNGNTATGCTNTNGCTNNATCTTATGNTCTCTTCTCTCNTCCTNTNNNTNATGTNATTGTATAT
NTCCGATATCCNTACNNTGGCTCGCC
GNGCCTNGTGGGNNTTNTNNANNTATATTNANTTGNNNNNNNTNNANNTNCNCNTNCTTNNNTCTTNN
NGNNNCNTTCCCNTNTNTCTTNNNNGGNNGNNTTTNGNAGNNTTNNATNNTTGAGCCNNNNNGATCG
TATTACAGTGGCGGCCGCTCTANAACTAGTGGATCCCCCGGNCTGCAGGCCGCTTCGNGATCTGNGGN
CCCCGGTGCAAACATGGCCAAGTCCAAGAACGNACNCCACGGACANNCAGTCCCGAAAATGGCNTAN
AAACGGCATCAATANACCCCGATCACAATGATATGAATCTNTTAAGGGGGTANACTCCANGTTCCTGA
GGAACATGCGCTTTGCCAAGAAGCACAACATAGAAGGNGCCTNNAANANGATGCNCGCNNNCCAATG
NCAAGGCCATGAGTTGCACCGTGCTGANGCTGTCAANGCCCTCCTGAAACCCAAGGANATCANNCCCA
AGATGCCAACAGGTGGNAANTCGTAAGCTCAACCNACTTGCCTACATTCCTTACCCCAAGCNGGNNAA
NCGCGCTCGTTGCTTCGNATCTGCCAAAGGGGCCTTNATGGCTCNTGNCCGGGTCCAAAAAGATNCGT
AGGGTCAANGGTCTTTNNAACCCATTGGNCCAANGCCACCTTGGCNNNCTTGCCTNNTTNACAAACTT
GGCCAAAGGGGTTGGTCNNNTGCGCCNCCAANCGAAAAAGGTTTNCCCGGNGANGNTAANAGNAACA
CTTTTATTTNTGCCCAANGTTGTGNGATNGACCAGNAAANNGNAACNTTGGGNNGTAGGAACCCCCCC
TTGGNGCCCTNGCNTTGNNCNCTGGCGATNGGGNGNNCNTGANTGNTNACCNCNCTTGGTGGCCTANT
TATTGNCGNCCAAAAATTNANATATCTCGCGGCACCTGGGGTNCNTNNTNNNGNNTTCTNTTAGAAGT
ANNTNCCCCTTTTGCGNNAGGTGGGGGGGGTGNCNCGCGTATANACCCTTNTNTTCTCGTCCCTTCTTT
ATATTGTGANGNCTGGNTNATANTTCTCAATACNTGNACNTCTNTCTTGGNCTTGCNTNGGANTTC
NTNNNACANTAANTCTNCNNANNTAGTATTTTNCNAANAGAGTTACTCGATACTTACACCCNTNGAAA
GNTNNCNNGATTTANTGCNTTCTTNNTCTTTTTNNTNTCTCANNTACGTGNNGNTANCTTNTNNTCNGT
TGTCNCTCTCCCNTCNNCNGNNTTCTATGTANNNNNNNTNGTTNTTACGNTATNNTGTCATNACGTNAC
NTCTCTCNGNTATGCTNTNGCTNNATCTTATGNTCTCTTCTCTCNTCCTNTNNNTNATGTNATTGTATAT
NTCCGATATCCNTACNNTGGCTCGCC
TNNNNNAAAAGCTGGAGCTCCACCGCGGGTGGCGGCCGCTCTAGAACTAGTGGATCCCCCGGGGTGC
AGGCAGGATCAAGCGATTCCTGGCCAAGAAACAAAAGCAGAATCGTCCCATTCCTCAATGGATTCGA
ATGAAAACTGGCAATAAAATCAGGTACAACTCCAAGAGAAGACATTGGAGAAGAACCAAGCTGGGTC
TATAAGAAGCAAGCTGGGTCTATGAGAAGTGGTCTTAACATGTANACCACTTTTTTGCAGCCAGAT
CACAATGAAAACATCACTACTGTAANGCTNGGCCCATGATGTTATTTCCTCACTATCAGTCTGAGACCC
AGCATTAAATATAAANCGTTGCAAAAAAAAAAAAAAAAAGGGGGGGGGCCCGGGCCCCAATTN
CCCNTTNNGGGGGGNNNTTTTAANANNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNC
TTGANANNTTGGACTCCCCGCGGNGGCGGCCGCTCTAGAACTAGTGGATCCCCCGGGCTGCAGGATT
CGGCACGAGGCCGNNCTTGCCTGCTGCCTGCCTGCCTGCCACTGAGGGTTCCCAGCACCATGAGGGCC
TGGATCTTCTTTCTCCTTTGCCTGGCCGGGAGGGCCTTGGCAGCCCCTCAACAGGAAGCCTTGCCTGAT
GAGACAGAAGTGGTGGAAGAAACCGTGGCCGAGGTGGCCGAGGTACCCGTGGGAGCCAACCCCGTCC
AGGTGGAAGTAGGAGAATTCGATGATGGTGCCGAGGAAACCGAGGAGGAGGTGGTGGCCGAAAACC
CNTGGCAGAACCACCACTGCAAACACGGCAAGGTGTGTGAACTGGACNANAACAACACCCCCATGTG
TGTGTGCCAGGACCCCACCAGCTGCCCTGCCCCCATCGGCAAAGTTTGANAAGGTGTGCAGCAACAAC
AACAANACCTTCAACTNTTCCTGCCANTTTTTNGCCACCAAGTGTACACTGGAGGGCACCAANAAGGG
CCACAAACTCCACCTGGACTACATGGGGCCTNGCAAATAGATCCCCCCTTTGCTTGGACTCCGAGCTG
ACTGAATTCCCCTTGCCCATGCGGGNACTGGNTTAAAAAACNTCNTTGTCCCCNNTTTCANAAAGGG
GAAAAGNAAAAANAACTNTTCTNNCCCGAAAANCCAAAACTTTGCGAAGTGNAAAAAATATCCCCCC
GAAAANTTGAAAAAGCCCCCTNGGGGGGCTGGGCAAACTTTTNTGGGGNAAATTTGGTGGCCCCGG
GGATTTTCNCACGNNCCNCCCCNCAAANGTGGACCCTTTTTCCCTTGGGCNCCTGGGNAANTTTGGGG
GCAANTTTNGAAANCAANCCCCCCCNGGNNGGGGTCCCTTTTTTTNAAAACCNGANGGTTGGGGN
NAAANNNGNNCCCCCCCCCCTTTTTTTCCTTATNGGNAAT
TTNNNAAAGCTGGAGCTCCACCGCGGGGGCGGCCGCTCTAGAACTAGTGGATCCCCCGGGCTGCAGG
GGAATTTGGTCTCGCGCGGGTCAAAAGCNNCGNGACCAAGACTATGAANGGTTTTGAATACATCTTGG
CCAAGCTGNAAGGTGAGGCCCNTNCCAAAACCCTTGTTGAAACAGCCAAGGAAGCCAAGGANAAGGN
CAAGGAGACGGNNCTGNCAGNTACAGAGAAGGCCANGGACCTTGNCAGCAATGGCAGCCACCAAGA
AGCAGCAGCAGNANCANNAGTTCGTGTATCGAGCCCANGCCCTGAAGCATCANNCTAGCAGACTACC
AGCTCCAGGCCNTGNGCCCCCTNCTCTTGTACTTTATTATTAAAAGCCAGCTTNTATCCCTNTCGACTG
TCTGGGTGNTGGGCTGAGGGTANGNGCCCTGTTTGGCATTTTCAGCATAGCCAGCTGGTTACCCATATT
TGANCCGGGCTTACTTACCCCTGCATNGGGTGNCTGAGGACACATGCNCATNGACTTGCCCACGGNCA
CCGTATNCANGTNANGCGGTCNTGAATGCTATACTGTNNGCTACATGANGNCANGGANCGTCGNTNN
NTCCTCTGATGAGAACGAANTATTTGAAACAAAAATNTTGNGTTGTNACANGCNCNCAAANNATACTT
TGACGGAATTCNATNNGACCCTTTNTAACAATTAAANCCTTNNNAACANAACCNCTNGGGGGGGGGG
CCGGGGGCCCAACCCCCCCCCNTTATTTTGGGGGNCCCCCCNTNAANCANNAAAGNCCCCNTGGTTAA
AAAACCCTTTTGNCCTTATTGGGNTTTCCGGGGNTTTNTTNTTACCCTGGGNCCCCATTNTTTANTTTNA
AAAANAANGGGGGACCCAAANCCCTTNTTTTTNCCANGNCCCCTTTTTNTTTCCCCCTTCCCNTTTTG
GGGGTTTTTNTTTTNAACCTTTTTTAAAAGCCT
NAAANCTGGAGCTCCACCGCGGTGGCGGCCGCTCTAGAACTAGTGGATCCCCCGGGCTGCAGGGGAA
TNTGGNCTCGCGCGGGTCAAAAGCNNNGNGACCAGANACTATGAANGGNTTTGAATNCATCTTGGCC
AAGCTGNAAGGTGAGGCCCNTCCCAAAACCCTTGTTGAANCAGCCAAGGNNGCCANNGAGAANGCCA
NNGAGACNGNNCTGNCAGCTACNGAGAAGGCCAGGGACCTTGNCAGCNAGGCAGCCACCAAGAAGC
AGCAGCAGNATCANNACGTTCGTGTANCCAGCCCANGCCCTGAAGCANNAANCTAGCAGACTACCAN
CTCCAGGCCNTGNGCCCCCTTCTCTTGNACTTTATTATTAAAAGCCANCTNNTATCCCTATCNACTGTC
TGGGTGGTGNNCTGANGGTANGNGCCCTGNTTGGCATTTTCANCNTAGCCAGGTGGGTACCCATATTN
GANNCGGGCTTACTTANNCNNGCATANNGTGGCTGAGGACNNATGCNCANAGACTTGCNCACGGCCA
CNGTATTNACGTNAAGCGGNNTGAANGNTATACTGTNNGNTACATGACGTNACGGNNCGACCGATA
NATCNTCTGATGANAACGGANNTATGTTGANACAANATNTTNTNTGTTNCANGCNCNNAATANATACT
TTGACCGAAATTAATCTGGACCCCTNTTACCAATTAAAGCCTTGNCCCCCACCNCTGGGGGGGGGGCC
GGGTGCNGAACCNCCAACCATTGTANTTGGNNCCCNNTAACCCCCCTTANAAANTCCCACTTGGTTNA
AACACCTTTTTGGNCCTATTTGGGNTTTCCGGGAGTTTNTTTTTAACCTGGAGCCCCATTATTAGTTTAA
ATAAAAANGGGGACCCGAAAGCCTTNTTTTACAAAGNCCCTTTTTNTTAAC
GNCCCTTANTAAGTGNAGTCNGANTTNANAGGGGAGNCCGNATTANANNAACTACGTTGGATCNCCC
GGGCNTGCAGGCAATTCGGCACGAGGGCCCCCTNTTGAAGCNCNTGCAGTACTGAGCNCCNAAACNGNNA
TNTATCACTGTGCCACCCANGGGCCCGNACNNTAGAGANNNNNANCATNTNNCAATNGGCTAAAAAC
NGCTCTTTNNANCGATCTNNGTATTATTTACNNATGNNCAAACGCTGTNTNAGTTANAANTCTNNTGN
TTNACTATTNTTNATTACNTNTTNNNGNNCTNNCCANNNANAATGNTNCNTNNANATNANCNGNGNNC
TNNNTTANGGNTACTNCTCANATNGGCTTNGGTGTNNACNTGAACTNTGNNANTNGNAATNNCNNAG
ACCGGTNNNGTATATTANNANCATNTNNGNGNNTANCTTTCTNTCATCANNANNNTATCCNNAGCGNN
TTACNTANANATANAAATNNNAAAGAGNGNTNNTTCCTGGNACTNAGTNAAGANNNNGAGAANCGN
NGATNANANTGACATGNNNNANNCNCANNANGNNTNANGNANTAAGTTNCGTNNNNNNGANNNNGN
NGCNCTACCTNATTNANTNCGNGNNGNNNCNACNANNTANANAGNTTNGNANNNNNNNANNNTNGN
NGNTCNNNNCTTCNNAATGNNGGCANANGGANNTACTNNTNCTANNNANGNNGGGNTGNNGNNCNN
NTTTTCTNTNGTNTTAATNNTAGTCNNTANAANGANNTATNTNANNNTNAGGNNNCNNNANCNGNAN
NAAANTAATGTACTNNNNAAAAGNCAGAANNNNTGGNTNNNNATNTNCNCNGTCGANTACCTNCTAG
NCNNCANNTTAANNTTGAGGNGAGGNCNTCNGNNNCNNAGNNCTNTCNTTAAGTNGGGNNTGNNCGN
NGCNGNNTANTNNNNTNTNNNTTTATCNTAGNNGNNNAGCNTANNGGACANNNANGCNGCTNNTNAN
NTANGTCNCGCNAATNNNANNNCGNGANATAGCNNATNTNTTATANNNTNNAGCANCAGCACTANNG
NNCTNNNATNAAANNTNTTTTNANTGCTCNNGNNTNNTAGNAGGTNNATNNNTNNNAGNATTNTNNC
NNANNNTGTNNNNAGGAGCANNNTGCNTGNNTNTGCTNGATATGNTCACTGTGAANCN
TTNAGCCCTTATAGTGAGTCGTATTACAGGGGCGGCCGCTCTAGAACTAGTGGATCCCCCGGGCTGCA
GGCTGGACCCCAAGGCCCCCGTGGTGACAAGGGTGAGACAGGCGAACAGGGCGACAGAGGCATTAAG
GGTCACCGTGGCTTCTCTGGTCTGCAGGGTCCCCCCGGCCCTCCCGGCTCTCCTGGTGAGCAAGGTGCT
TCCGGAGCCTCTGGTCCTGCTGGTCCCCGCGGTCCCCCTGGCTCTGCTGGTTCTCCCGGCAAAGATGGA
CTCAATGGTCTCCCAGGCCCCATCGGTCCCCCTGGGCCTCGAGGTCGCACTGGTGATGCTGGTCCTGGT
GGTCCTCCCGGCCCTCCTGGACCCCCTGGTCCCCCAGGTCCTCCCAGCGGCGGCTACGACTTGAGCTTC
CTGCCCCAGCCACCTCAAGAGAAGGCTCACGATGGTGGCCGCTACTACCGGGCTGATGATGCCAATGT
GGTCCGTGACCGTGACCTCGAGGTGGACACCACCCTCAAGAGCCTGAGCCAGCANATCGAGAACATC
CGGAGCCCCTGAAGGCAGCCGCAAGAACCCCGCCCGCACCTGCCGTGACCTTAAGATGTGCCACTCTG
ACTGGAANAGCGGAAAATACTGGATTGACCCCAACCAAGGCTTGAAACCTGGATGCCATTAAAGGNN
TTTNTGCANCATGGNAAACCGGGNGAAAAACCTGTNTNTACCCCANTTCANCCCANCNTNGGNCCCAN
AAAAAAATTGGTTTTTTNAANCAAATAANCCCCAAAGGAAAAAAAAGGCNCTTTTGGGTACNGGGAG
AANAACNNATGACCCGGGNGGNATTCCATTTNTGAATTTTGGGGGCCCANGGGTCCCNATCNCTCCCN
TTGGGGCCATTCNANNTGATTTTCNNTGGCCNTNNNNNCNNCCCGNGGCCNTCCCAAAAANNTTTCCC
TTACCCTCNNGAAAAAANNGGGGNGNCCTANNTTNGNCCNANNNAANTTNGGCNANTNTANNANAAG
GGCCT
GCCCTATANNGAGNTGTATTACAGNGGCGGCCGCTCTAGAACTAGTGGATCCCCCGGGCTGCAGGCGG
GGGTCTCCGGCCCTGCAGGCGCAAGAGGAGGCGGGTATGGGCCCCAGGTCCCCCTGAGCCAGCCTCCC
GAGGAGGAGCTTGAGAGGTTGACCAAGAAGCTGGTGCACGAACATGAACCACCCCGCCNANCGGGAA
GTTCTTTCGGCCGCTTGGGNGGGTTGCGGAAAAAAACGTTGNTCGGGGAATGGGNCCGGGGTNGGGG
CCTNGANTCGCGNTTTTCNACNTGGCTTGTTTTGGGNGTTTNACNNNCNGGCCCNACTTTCGGGNCNA
NATTTTTNNCCCCGGGAAAAGNAGGNCNNNTNGNAAAACNNGTTNTGNGNCCNCCTNGNAAAANGGT
TCNNNNNNNTCCNANCCATTCTNGANCNGANTNTNGGGGCTATNGGGAAAGGCTNNCAACCNGGCTN
GTTTAACTNTGNGGTGTGCCACCNGGGGCTCTAAACGGATTTCTTTNACTGTGGATANCGACANAACC
ACATATCACTGTGATNTGAGGACANTTCACANAGANAANTTTNCGCCCCAAANATNCTCNAANATGTG
GGNGNGGGCNCATATATGTCCCCNAAAACACANNGTNCNGAGANAGAACACCTGGCGAAAATTNATA
NTTCTTGTGATANCAGNTTTTTTTNCAATNTGGNGTGTTACNAAAANGTTGGAGNAAGAAGTTGTGGCT
TNTTGGTTCTTCTTTCTTTAGNNGNGAAGAAGTTNANNANGGGTNTTAANNNCACTGGTGNTGAGGGA
CCATTTTTTTTGCTANNAAAATTCNTNNNTGNCTTNNTGGGAATANNNANNAAGGNTCTTTNTANNA
CCTNTTGGTTCAACCCNNCGNGGTTGNTTGAATTCCNCCAAGNNAGGAACCCANNCTGGTGGGCCTGC
TTTTTTTTANTAAAACAAAGAATNCTTCTNNNNNANAAAACNNCTTTTTTAANANTTTTTNACCAAAAA
GANN
TTNAGCCTATAGTGAGTCGTATTACAGTGGCGGCCGCTCTAGAAGTAGTGGATCCCCCGGGCTGCAGG
ANTTAANAGCGAGGNATNTNCGNNTGAGNGAANGNANANNGCTGCCTNCGNGTGTGNGNNNACNNT
AANNANGTCCTATTTAATTTAGGNNNNTNTGTATCTGGGNNACNATNTNNNTTCTANNATAAGNGACA
CCATTAAACGTTCGTTNTTNATATATACNAANNGNNCTTTTTGCCGTGGANGNGGGTTCCNATNTGNC
CNCCCNCNACCCTNGNNCCTNTTTGCAAAGCNTCCCTNGTANCNAANNNAATTCCNAAAANGGGNCC
AAANCANTNGGGGGNCACAAAGAACCAACTNGCGGTNNCCCCCCGGGTTTCCCCANCCCAGGGTTNCA
AAAATTTTAAAACNTTNAATTTGTGGGNAAAAGGNATTCNNCATTGGAGANNNGCCTTTAACANANG
GGCAGGGGCCCAANNCNNNACNTTNTTTTNTNGGNTGNTGACNTNTNNAAAANNCANANACGGNTAT
TTGTTNTCNAAAGGNGGGGANAAAAAANCCNCCCNGGNGANATGNAACNTCTTTTNNCGGGGGCNCC
TGGAAANAGNCTTNGTAATTACNAAAGAANCNGCTCTTNCCNTCTNATNNACANNTGNGGNAACCGN
GGGTANCNACATTNGNANTAATTTTNNGCNNANTNAAAACCTGTGGGNNGNGNGGTTNGTGGATTN
AAACNACCAANNCNTCNTTTCTTNGGNGGGGGGGANNAAAAANNAAGACCCCCCNNNATANCTNNNN
GNAAAAAATNATTAAAAAAAGCNNANTCCACTNAATGAATNAATTNAATCCNCCCTTTTTTTTTNGTT
NGGGAAGGCCTCCCNTTTTTNCNGNNGNNTTTTNTNNNTTTTTTTCNNNNGNNNNGNTAANACACCCC
CCNGNTTNNGNNGTNTNNCCNCGGNNTTTNTCCCNNGAGGGGGGTTNNTTAAACCAANNTTNNCNAA
ATANNTTGGGGNCCCCCTCCCCNCGGNCC
TTNAGCCTATAGTGAGTCGTATTACAGTGGCGGCCGCTCTAGAACTAGTGGATCCGCGGGGCTGCAGG
ANTTAANAGCGAGGNATNTNCGNNTGAGNGAANGNANANNGCTGCCTNCGNGTGTGNGNNNACNNT
AANNANGTCCTATTTAATTTAGGNNNNTNTGTATCTGGGNNACNATNTNNNTTCTANNATAAGNGACA
CCATTAAACGTTCGTTNTTNATATATACNAANNGNNCTTTTTGCCGTGGANGNGGGTTGCNATNTGNC
CNCCCNCNACCCTNGNNCCTNTTTGCAAACCNTCCCTNGTANCNAANNNAATTCCNAAAANGGGNCC
AAANCANTNGGGGGNCACAAAGAACCAACTNGCGGTNNCCCCCGGGGTTTCCGCANCCCAGGTTNCA
AAAATTTTAAAACNTTNAATTTGTGGGNAAAAGGNATTCNNCATTGGAGANNNGCCTTTAACANANG
GGCAGGGGCCCAANNCNNNACNTTNTTTTNTNGGNTGNTCACNTNTNNAAAANNCANANACGGNTAT
TTGTTNTCNAAAGGNGGGGANAAAAAANCCNCCCNGGNGANATGNAACNTCTTTTNNCGGGGGCNCC
TGGAAANAGNCTTNGTAATTACNAAAGAANCNGCTCTTNCCNTCTNATNNACANNTGNGGNAACCGN
GGGTANCNACATTNGNANTAATTTTNNGCNNANTTNAAAACCTGTGGGNNGNGNGGTTNGTGGATTN
AAACNACCAANNCNTCNTTTCTTNGGNGGGGGGGANNAAAAANNAAGACCCCCGNNNATANCTNNNN
GNAAAAAATNATTAAAAAAAGCNNANTCCACTNAATGAATNAATTNAATCCNCCCTTTTTTTTTNGTT
NGGGAAGGCCTCCCNTTTTTNCNGNNGNNTNTNNNTTTTTTTCNNNNGNNNNGNTAANACACCCC
CCNGNTTNNGNNGTNTNNCCNGGGNNTTTNTCCCNNGAGGGGGGTTNNTTAAACCAANNTTNNCNAA
ATANNTTGGGGNCCCCCTCCCCNCGGNCC
TTNANCAAAAGCTGGAGCTCCACCGCGGTGGCGGCCGCTCTAGAACTAGTGGATCCCCCGGGCTGCAG
GAATNCGGCNCGAGGNTGAAAGNAGGANGCTNTTGNNCTTNNTNNGNACATTTGGNGANGCNCACAG
AATGGGCTTACTTATTNTNNANAANGGANTGAANAANACNNGNCNACACAAANCACTGANCTAGCGT
TAACACGGAAGAGGCGCTGATTNCAGATTNNAAGTCGGGTNCTGTGTGCGTCCANGNAGTACACNTTN
GNGCTTGNGAAGGGCTTGNATGGTGCTCACCTNAAAGCATNAGGGANCCANNAANAANGNNATCAGN
TGCTGAGCTNGGTNAATGTNANTNGAACTNATTNTTTGNNNCACGNGAAATTGNTCTTTGGTATGCCC
CNTTGNANTGANTAAGACCTGTTCNNAAACTTGGNANCTTNATNCATAATGCTGAAGGCAGAAAATCT
GTTGAGGANAATTGCANGAGGGAAGGCTGCTTTGAAAGCGCCTNATTCTTTTTTCCAAACACTGCTTC
ATTCNCCAATATNAATATGATCTTAAAAAGTGTGNAAATGAATANTGNTTAANGTCCCAATTGTGCTT
TTAGAGGGGGGCGNACTGATGCTTAAAAAATGAATTTTTGTTNTAATTACAAANACTGGTATTATNCN
GGGGAAAAAGTGGGTTCAGGGTACANTCTNGGGGAAAAACCNACCCGTNTGCCTNTTNAANCCTAAA
AATTGCATNAACCTTTATNTTNTGATTCAAAAAAAAAGGGCNCNCTTTTGGNTTTTTAAAAGTTTNTAN
AAACCCTGGNCCTTTTTAAGGGGTNAATGNTTTNAAAAATNGGGGGGGTTGTTACCAAACAAACAAGT
TNNTTTTTGNAAGGGTTTNGGGTTTNCCCCNTTAAANNGGGGGGGNGGCCNGGGTTTANCCNTCNA
AAAAAANAGNCCTGGGGTTNNCCNT
GANCANAGCTGGACTCCACCGCGGTGGCGGCCGCTCTAGAACTAGTGGANCCCCGGGCTGCAGGTTCC
TCTCCAGGTCTTTNNTGNTGNCAGAGGTGGGGGTACGGTCCAAACCCCATTAGGTCACTCCGGCAGCA
TGTGCTTTTAGCTTCTCTCTTGACTAGGAGTCACATATCCCTTTGTCAGCTGGCCTTCAGCTCCTTNGNT
TCTGNGTATAAACCTTGGATATTACNGNNTTTTATATTTACCAGAGCTATTGGAGCAANAGTATTNGAA
CCACTAGCCTTTGAAATAAAAGNCAGCCCCGTTATTAACAGNGCAGATACTGAGTCCACTTGGATTTC
TTGCAGCTTTCTTTGCACTGCTTTAGGCAAAAAGNGTTCATCTGGTTTTTTTCATTTGACACCCCAGTTT
GCTGCANNGACAGAACTTANTGCCTAATCTGTAGAGTTCNTATATATNTATATAAAATATATTTNCCCN
CNCTCCNCATATATATATGAAGTTTAATGAACNGGNCAAGNGAAACATCCTAACTGTCCTTGGGATAA
AGGANCCAGTTTTGCAGGCAGCCAGCGCGGGGTTTTTNTGTNGGTCTCCCANCACATGCCCATTCCTTT
GCCCGCCCATTCCTGGTACACTCGANGGGAGCTGGGGGACNCCACTGGGAANGTTTCTGNGGNCCTGT
CTTGAATGNGGCAGGCTGGAGGGGNCCCCGCGAAAGNGANCTGGGGCGTTTTTTGTTCCCCACCCNTT
TTAAACTGGTTACNGGCAGGGGTNGCGNCCCNCCCCCCCCCGTCAAAAACAAAAGTTTAAAGGGGGA
ATTTTGGGGGGNGNACCCTTTACCCCCCGNAAAAAAAANCTTGGGGAAANCCNGGAANNCCGGCTTT
TAANNAAATTTTGGGNGGCNTTTNAAAAAAAAANNGGCCCCN
TTNNNNAAAANCTGGAGCTCCACCGCGGTGGCGGCCGCTCTAGAACTAGTGGATCCCCCGGGCTGCAG
GAATTCGGCACGAGGGGCAGGCCGCGGTGAAGAAGATCCAGGCCTCCACCATGGCCTTCAAGCAGAT
GGAGCAGATCTCCCAGTTCCTGGAAGCAGCCGAGCGCTATGGCATCAAGACCACTGACATCTTCCAGA
CTGTGGACCTCTGGGAAGGAAAGAACATGGCCTGTGTGCAGCGGACACTGATGAATCTGGGCGGGCT
GGCGGTAGCCCGGGATGATGGGCTCTTCTCTGGGGATCGTAACTGGTTTCCCAAGAAATCCAAGGAGA
ACCCTCGGTACTTCTCGGACAACCAACTGCAGGAGGGCAAGAATGTGATTGGCTTACAGATGGGGACC
AACCGCGGGGCTTCTCAGGCAGGCATGACGGGCTACGGGATGCCGCGCCAGATCCTCTGATCTGCTGT
GCAGCCCTGCCCCTGCCCTCCGTGAATGGTTAATATATATGTNGGTTTTTTTTTTTTTAAAAATTAANTT
TCCTTGANAGCCCTNGGANGCTTTCGCNCTTCCNTTTTGCAAGGGTTGGGGCCTGGCGTNTTTTTTTCC
GGGGGGGCCCAACGGGGGCCNCCCCCTTTGGGNTAAANAAAAAAATTCCNTTTTNCAAANCCCCCAA
AATNGGACCAACNGGCCTTTTTTTTTCCCCTGGACANAAAAANNGGGGGGGACCTTTACCCGCTTTTC
GGAACNAAAACTTTTTTTTTTTTTCNTNGGGCCCTTTTNCCCTTTTGGAACCCTTGGGGCCCNAAAACC
ATTTNNTTAAGGGGGGGCAAAAAAAGGATNNTTTNNTTNAAAGGNTNTTTTTCCCCCCCCNAAAANTT
TNNCCTTTNCCCCCNCAGTTNGGGGGGGGGGGGGGGCCTTTANTTTTTTTGGGGGGGGGGNCCTTGGG
NCCCCCCCCCCCCCCCCNC
AAAGCTGGAGCTCCCCGCGGTGGCGGCCGCTCTAGAACTAGTGGATCCCCCGGGCTGCAGGAATTCGG
CACGAGGATTCATTCACACCAACAACACAACTATCAATAAACCTAGGCATAGCCATCCCCCTGTGAGC
AGGAGGGGTAATTACAGGATTCCGCAATAAAACTAAAGCATCACTTGCCCATTTCTTACCACAAGGAA
CACCCACTCCACTAATCCCAATACTAGTAATTATTGAAACTATCAGCCTTTTTATTCAACCTATAGCCC
TCGCCGTGCGGTTAACAGCTAACATCACTGCAGGACACCTATTAATTCACCTAATCGGAGGAGCTACA
CTTGCACTAATAAGCATTAGCACTACAACAGCTCTAATTACATTCACCATTCTAATCCTACTAACAATT
CTAGAGTTTGCAGTAGCTATAATCCAAGCCTATGTATTCACTCTCCTAGTCAGCCTATATCTGCATGAC
AACACATAATGACACACCAAACTCATGCTTATCATATAGTAAACCCAAGCCCTTGACCTCTTACAGGA
GCTTTGTCTGCCCTCTTAATAACATCCGGCCTAACCATGTGATTTCACTTTAACTCAATGACCCTGCTA
ATAATTGGCCTACCAACAAATATACTAACCAATATACCAATGATGACGAGATGTTATCCGAGAAAGCA
CCTTTCAAGGGCACCATTACCCCAGCTGGTCCAAAAAGGCCTCCGGTATGGAANAATTCTTTTATTATC
TCCGAAGTACTATTCTTTACCGGATTTTTCTGAGCTTTNTACAANTAAGCCTTGGCCCCCANCCCTGNA
CTANGNGGGTGNTGGACCCCCAACAGGCNTTCNCCCCTTAAACCCCCCTAAAAGTCCCACTGGTTCA
NGGNNGGCNTTTNNNNNNNNNNNNNNNNNNNNNNNNNNNNCNCNNNCNTNCNNNNCNCNNCNTCC
NNNCCCNCTCCTNANNGGNNNNNANNNGTTTNCNCNNNNCCTTCAGCCTNCNTGATCGTATTACAGTG
GCGGCCGCTCTAGAACTAGTGGATCCCCCGGGCTGCAGGTTGACATCTATGAGGACTTCCCGGATCTC
AACTCGGTGCTCTGGGGAGCTGAGGAGAGGAAATGGGTCCCCTACCGGGTGCCCAACAATAACCTGC
CCATCCCTGAACGCTACCTCTCTCCAGATGCCAGGGTATCCACGGAGGTCCGGNCCATCATTGCCTGG
ATGGAGAAGAACCCCTTCGTGCTGGGGGCAAACCTGAACGGTGGCGAGCGGCTTGTGTCCTACCCCTA
TGATATGGCCCGCACACCCAGCCAGGAGCAGCTTCTGGCTGCGGCCATGGCAGCGGCCCGGGGAGAG
GATGAAGAAGAGGTATCTGAGGCCCAGGAGACCCCAGACCACGCCATCTTCCGNTGGCTNGGNCATN
TCCTTCGCCTCTACCCACCTTACCATGACTGAGCCCTACAGGGGAGGATGCCAANCACANGACNACAC
TGGTGGCATGGGCATCCNTCAATGGGGCCAANTGGAAAANCCCGCTTCTGGGAACTATTANCCGACNT
TCAGCTACCTTGCACACCCAACTGGCCTGGAGCCTCNTCCATTCTTCACTGGGGCTGTNGATCAANTTC
CCCNCATNGANAAGGCNAGCCTTGCCTCCCCTAANNGTTGGGNANTAATCAATACNATAGAAAGNNC
NTNNTGCCTCANCCCTTTCATGGNNAATCCANGTTTNTCTCCGTGGGCATCNANNGGGGGNGNNCTNG
NGACCATCACCTANNCCAAAGGGGCCATCCCCCTNAATANCCCNNATNNCCTCCCNTTTTCCGNGTG
CAGTNNGGNATTTANNCCCCCGGGGGTCGGGAAATCACCNTGCCCNTCGNGNTTNNCNATATNNGCCT
TGNGCNNNCATNTCCNTNAAACCNCCNGTTNGANNATNTCNCTGNCGGGANCNGNNCNNCNCNTCNG
TNANGGNTCTTANCNCCTCTCTCTCNCTGTTCANANANACNTCTGNCTANNNCGTGTGAANTCANNGA
NACNTTCGCCNNTNCNCCTTGTNTNAGCGCNANNTTNTTGCACNTNTTNNGGCTCCTCTCTNCCNCNNA
TAGGGTGAGACGCTNNCTCNCGCTGNTTNGATNCAGTGTGNCTCATGTNNCATNTGNTCNCACCC
GCCTATNNGGAGNTGNATNNCNGTGGCGGCCGCTCTAGAACTAGTGGATCCGCGGGGCTGCAGGAATT
CGGCACGAGGATCGTATAGAGGAAGTTCCTGAACTTCCTTTGGTGGTGGAAGATAAAGTTGAAGGCTA
CAAGAAGACCAAGGAGGCTGTGTTGCTTCTGAAGAAACTTAAGGCCTGGAATGATATCAAAAAGGTC
TATGCCTCTCAGCGAATGAGAGCTGGCAAAGGCAAGATGAGAAACCGTCGCCGTATCCAGCGCAGGG
GACCCTGCATCATCTATAATGAGGACAATGGTATCATCAAGGCCTTCAGAAACATCCCTGGAATTACT
CTGCTTAATGTAAGCAAGCTGAACATTTTGAAACTTGCTCCTGGTGGTCACNGTGGGACGTTTGTGCAT
TTGGACTGAAAGTGCTTTCCGAAAGTTAGATGAGCTATATGGCACTTGGCGTAAAGCAGCCTCCCTCA
AGAGTAACTACAACCTCCCTATGCACAAGATGCTCAATACAGACCTTAGCAGAATCTTGAAAAGCCCA
GAAATCCAAAGAGCACTCCGAGCACCACGCAAGAAGATTCATCGCAGAGTCCTGAAAGAANAATCCC
TGAAAAACCTGAAAATCATGTTGAAGCTTAATCCATATGCAAAGACCATGCGCAGGACCCCATTTTTT
CGANAGGCCAGGGAATCACAAAATCCGCATGGATAAGGNACAGCAACCCTTAAAAGCCAAATCAAAT
CNNAAAGGGGTTCAGGGCCAAAAAACCTTGNNGNTNGGNAAACAANGAAAAANAAAGGGTGTTGGC
GAATAAAAAACCTGAAAAAANCCTNGGGGNGGGAAAAAAAAGGNTGCNNGGGGACCCAAAAAACCC
CCCCTNTTGAAAANAAAGCCCCANAAAAAAAAACCCCGCTGANANGAAAAAAAANGNGNNGTGGNG
NAAACCTNAAAATTGGTTTATTCCATAAAAGGCCCAATTTTTTTGGGGAACGNNN
CTNTNAGANGGGTGGANGTNTGCNGTGNGNCNAACNNNNTCGTNTACNGCCCTCATCAGGGAGCCCG
NATNACATGGGCNGGCCGCGCCCCAATAACTTAGCCGGCTTCCCCCNCGCTTCATTTTCNTGCAATGG
CTCANATCNNTNTAAATCTGTNCCTCCGNTNTGCNTGCNATCNNNTTANCTGTGTCTCTCGGNGNNNN
CCNCATCACGGNCANCAGNCNCNTCATGCANAGGGCGGANACNCCCNCTNNTANCNCTAGNCNNNGT
NNNNCTCCCCCCCNNNTTCGNCNTTCNCGCNATATATGGNNTNNAGANTTTTTGNATNGGNNGGCNCT
CCCAGACACGCNTTTNTNTCAANTNCGNTNCTNNGAGACNGNANCCGGNNTCGCNGCNTTAATNNACT
AGATNTNNCNNNNGNGNCANTCTCNNANANTCATNTNCNTCTTATCTNNANGCGGNNGGACNGAATG
NNTCNAATANATTCACTAGCGGACGNTNTNGTAAATAGACTCNCTNNANNGNNNTNGNNTTCCCTNGT
NCTGGCNCNAANNNTNTTNATCTNNGANTNNGTATACTCCATCTCNNTNNANNATTATNNGNATTN
TNTGANNNANCGNCNNNANCGCGNGNNCNTTNTGNTNNGTCNCANCCNNCTNACCNCCNNGNNACGA
NCGGGTNNCTNCTAGTCANNNACNTTNNATCNNTCAAGTCCGAGNTACTTNNATNGTNTCCNCTNANN
ATNTNNTNCNGNTATNNNAANTTTGTCATGCANNTANNGCCTACTNGCGCGANNNNTCGNNNNNTCN
NNCNNGACCNNTNCTANTGATCNTTGNGCNTTNATCACTNNNNNGCCTGNCGCCNNNTNTCNGTNNCT
NTNTNNTGTGTNNCNCGACGTAGNCCNTCNTGTTNTNNNNTCGNNGCAACNNCTATNCCNTNGTGCTN
CNCGNCTNAATNNTGTGANTNCATNANCTCTTCNTCCATNTNCCCCNNCACCCAGGNNNCCCNNNCTC
NTCCTNTNNNTTCTTTATCTTAAACCTNNNCANNCACAGNGCTCCTTGTCNCNANCCGCTTCNNTTTTT
CTCTCNCGNCNNTGGNCTCNNTNNATCNTCTCTCTNTCTATNNCTTTACTTTNTNNNCGCNAATATGTG
GNGGCGNCGTANCTGNGATANNCNTACNAACTANTCTNATCTANTCNNNANAGNACAATAACTGCTG
ATGTTTGTANNNCGCTNNCACTNCANNCTNANTCCGGCTNTNNGGNNNTCNCAGNGTNTCGTCGTTNG
ANCANTATGACNTATAACTACNACTTTATGCTNAGTNACGANNGCTNNNAGATCATGANANTAGA
GTNANTANNGNANACNTCNTNANNCNAGTATACGNCNCATCNNCNTCNACTNGNTTNATGTNTCTNNT
CTTTNGTGATNCTANTNATNCCTNTNTGANNANTCNCANNNGCGCGATCCCTCGNANNNACAAGAGNT
ATTCTCGTNANATANTGNCACNCACCTCTNAGNGACTNANANCCG
TTNAGCCTATNTGGAGNTNNATNNCNGTGGCGGCCGCTCTAGAACTAGTGGATCCCCCGGGCTGCAGG
CNCTTCGGTATCTGCGGGCCCCGGTGCAGACATGGCCAAGTCCAAAAACCACACCACNCNCAACCAGT
CCCGAAANTGGCNTAAAANCGGNNTNANAAANCCGNGNTNNCANCNNTTTNANTNTTTTANGGGGGT
AAACCCCANNTTCNTGAGNANCNTNCCNTTNGCCAAAAACCCCACCAGAAAGGGCNTGAAAAAAATN
CAGNCCACCATNGCCANGGCCTTNATNGCCTNCNTGAGGTGTGTNAAGGCCTTTGAGAAGCCCAAGG
AATATCAAGCCCAATANGCCAACAGTGTGGCAGCGGNAACTCNNAACGNNTTTTGGTATATATCTCTC
ACCANACTNTGGAAAAGCGCTCTNGNTCTCATATCCANAGGGCNCTTNNTCTGTNGNGNACANNGTCN
CNGGCNAGGNTTTANCNNAGGNNNANCNCNNNTGGGNGTGGGTCTCCATGTNCNAGGGGTGCCCACG
CCCCCACCAAATCTGGNGAGTAGANCNTTTCTCTGCCAGTGTGAAGACAAANAAGTANTGGTGACACC
CCCTGGGCTTGTTNTCTGCGNGGNGCTGGNNTCTTGCTGTGCTNTTTCCACAAATNAATTNTGNANAAA
GAAAAAAAAAAAAAAAAAANNNTTTTGGGGGGGGGGGCCCGCCCCNAATTTCCCTTTTTTGGGGGGG
GGTTTANANNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNCN
TTNAGCCTATNTGGAGNTNNATNNCNGTGGCGGCCGCTCTAGAACTAGTGGATCCCCCGGGGTGCAGG
CNCTTCGGTATCTGCGGGCCCCGGTGCAGACATGGCCAAGTCCAAAAACCACACCACNCNCAACCAGT
CCCGAAANTGGCNTAAAANCGGNNTNANAAANCCCNGNTNNCANCNNTTTNANTNTTTTANGGGGGT
AAACCCCANNTTCNTGAGNANCNTNCCNTTNGCCAAAAACCCGACCAGAAAGGGGNTGAAAAAAATN
CAGNCCACCATNGCCANGGCCTTNATNGCCTNCNTGAGGTGTGTNAAGGCCTTTGAGAAGCGCAAGG
AATATCAAGCCCAATANGCCAACAGTGTGGCAGCGCNAACTCNNAACGNNTTTTGCTATATATCTCTC
ACCANACTNTGGAAAAGCGCTCTNGNTCTCATATCCANAGGGCNCTTNNTCTGTNGNGNACANNGTCN
GNGGCNAGGNTTTANCNNAGGNNNANGNCNNNTGGGNGTGGGTCTCCATGTNCNAGGGGTGCCCACG
CCCCCACCAAATCTGGNGAGTAGANCNTTTCTCTGCCAGTGTGAAGACAAANAAGTANTGGTGACACC
CCCTGGGCTTGTTNTCTGCGNGGNGCTGGNNTCTTGCTGTGCTNTTTCCACAAATNAATTNTGNANAAA
GAAAAAAAAAAAAAAAAAANNNTTTTGGGGGGGGGGGCCCGCCCCNAATTTCCCTTTTTTGGGGGGG
GGTTTANANNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNNN
NNNNNNNNNNNCN
TNNNACAAAAGCTGGAGGTCCACCGCGGTGGCGGGCGCTCTAGAACTAGTGGATCCCCCGGGCTGCA
GGTTTGTCACCACAACAGGCTGGTCATAACAAACACCCTTTTCCAACAACCTAGTAGACGTCTCTACA
CATGGACATCACCAGATGGTCGATACCGAGATCAGATTGATTATATTTATTTGTCGCCAAAGATGGAGA
AGCTCTGTACAGTCAGCAAAAACAAGACCTGGAGCTGACTGTGGCTCAGATCATAAGCTCCTTATTGC
AAAGTTCAGGCTTAGGTTGAAGAATAATNCCAAAANCGACTGGGCCNTTCAANGGGATTAANGANAA
GGATGCCNCAAATGANNAANCAAANTCCGTNTAANGCANATNGANAANNAAAACCNNGAGGTTACC
GTTCCTTCACCTGGGTCNTNAGTTCTTGGGGGGTCAGGCNTGANTAAGGAGGNGGGNGAACCATNTCA
GGAGCGAATTNTGTGTTCAGGCAGGATGAGAAAGACAANCCTCAAGCTAATGTCCCCTCAGNGGGG
CCATCACTTCTNGTNGGGCNAGGCGNTGAAAAAGGGGCCCTNGAAAAGNAAAANGGATGGCACCANT
TAAGTNTTTGACTTNGNCCTTAANCCCCTAAACNGGTTTGTTTAAAANACAGTGGGAANCCGGGTTTN
GGNAACCTTTTCCGGTTGGGTTATCNGGNAAACAAAACAAAANTTGTTNTTGGTTAGCTTGNTTTGNA
AAANAAATTTTNGGCAAATTAAAGNCTTGGACNTTTTTTNAANGGGGGAGNTTTTTNNTTTGGGANGN
GNAANGGGTTAACTGGNCCCATGGANNTTNATTGGGGCCNGGGGNTTTTTTGGATTTTAAAAGCCAAT
TTGGCCCCTTTNGGGNTTTTNGGATNAAAAAATTAGNCCCNGGGGNAAANCCCNTTTTTTTCCCGGGG
GGNTNCCCCNAAAAAAAAAAANNTTNCCAAANGGGTNC
TTTGANCACCGCTGGAGCTCCACCGCGGTGGCGGCCGCTCTAGAACTAGTGGACCCCCGGGCTGCAGG
CAGGANCAAGCGATTCCNGGCCAAGAAACAAAAGGAGAANCGNCCCATNCCTCAATGGATTCNAATG
AAAACTGGCANTAAAATCAGGTACAACTCCAAGAGAAGACATTGGANAAGAACCAAGGTGGGTCTAT
AAGAAGCAAGCTGGGTCTATGAGAAGTGGTCTTAACATGTAGACCACTTTTTTAAGCAGCCAGATCAC
ATTGAAAACATCACTNCTGTATTGCTTGGCCCATGATGTTATTNCCTCACTATCAGTCTGAGACCCAGC
AATAAATATAAAACGTGCAAAAAAAAAAAAAAAAANTNGGGGGGGGGCCCGGCCCCANTTCCCCTT
TAAGGGNGNTTTNNAAAAANNNNNNNCCNNNCCTNNNNNNCNNNNNNCNNNNTNNTTNTNNNTCC
NNCNNTNNNNNNNNTCTNNCNTNTNNNNNNTCNCCNNNNNTCTNCNNNNNCTCCCNNNNNNCCNNNN
TNTTNTNNNTTCTNCNNNCCNNNNTNTCNNNNNNCTCTCNTNNANNNTANNTTCTNNTCTNNNNNNCT
NNCTTNNTNNNNNNTNNNTNNTTTNCNTCNNCCTNNANNNNATCCTNANNANCTNCNNNNNNNTCT
NCCNNNCCNCCNNNNNNCTCNNNNNNTTNTCCNNNNNNNTNCTCNCNNNNNNNNNCNNCNCNTCCCCT
TNNNACNCTNTTNCCNNNNCCCNTNNNNNCNCNNNNNNCCNCNCTNNTNTTNTTTNNNTCTNCTCNN
CNCCCNTNTNNTANNNCCNNTTNTTTTNNNNNNCCCNTNTNNTTCNNTNNNNTTCCCNTNNNTTCTTTN
NNNCTTCNNCTNCTCCNANNTNCNCCTNTTNTTNTNTTNTTNNNNCCTTNTNNNNNNTNTTCNNNNTNT
CTTNNNNNTCNTCNNNNTNNTNCCNTNNTNNTTCCCNTNNNCTNNCCN
TTNNNNAAAAGCTGGAGCTCCACCGCGGTGGCGGCCGCTCTAGAACTAGTGGATCCCCCGGGGTGCAG
GAATTCGGCACGAGGCTATTCAGTGGAACTGCTGATAGCAATATGTCTTCAGGGAATGCCAAAATTG
GGCACCGTGCCCCCCAGTTCAAAGCAACAGCTGTTATGCCAGATGGTCAGTTCAAGGATATCAGCCTA
GCTGACTACAAAGGAAAATATGTTGTGTTCTTCTTTTACCCACTTGACTTCACCTTTGTGTGCCCCACG
GAGATCATTGCTTTCAGTGATAGGGCAGAAGAATTTAAGAAACTCAACTGCCAAGTGATTGGTGCTTC
TGTGGATTGTCACTTCTGTGACCTGGCATGGATCAACACACCCAAGAAACAAGGAGGACTGGGACCCA
TGAACATTCCCTTGATATCAGACCCCAAGCGCACCATTGCTCANGACTATGGGGTCTTAAAGGCCGAT
GAAGGCATCTCATTCAGGGGCCTTTTTATTATTGATGATAAAGGAATCCTTCGACAGATCACCATAAA
CGACCNTTCTGTTGGNCCGCTCTGNGGATGAAACGCTGAAACTAGTTCANGCCTTNCAGTTTACTGAC
AAACATGGGGAAGTGTGCCCAGCTGGCTTGGAAGGCTGGCAGTGATCCCATTCAAGCCTGATGTCCCA
NAAAAACAAAAGAATATTTTTTNTAAACANAAGTGANCCGCCTCGGNCCGTTTTAANGGNCCAGGCTT
GCAATAAGGCGGGCCTTTNAAAAACAAAAACCTTNTTTTGGGCCCTAATTTTTGGGGCNTNAAAACCN
TTGAATCTTTNCCCTCCCNNCCCCGGAATGNGNGGGGGGAACAAGAACNGGCCCTTTTCCTTGCANGG
GGTTGGGGGACCCCCCANCCNTTTTTTTCCTTTGGGACGGGAAANAACCTGGGCCCCNGGNCCCAACN
TGGTTGTTTTCCTGGGGANGGG
AAANCTGGAGCTCCCCGCGGTGGCGGCCGCTCTAGAACTAGTGGATCCCCCGGGCTGCAGGAAGCGA
TGGGCATCTCTGGGGACAACTGGCACAAGCGCCGCATACACACAAGAAGTACCGGGCCTTGAGGCTG
GACGTGGGGAACTTCTCCTGGGGCTCGGAGTGTTGTACACGCAAGAGAAGAATAATCGATGTTGTCTA
TAATGCATCCAACAACGAACTGGTCCGTACCAAGACCCTGGTGAAGAACTGTATTGTGCTTATCGACA
GCACAGCGTACCGACAGTGGTACGAGTCCCACTATGCACTGCCCCTGGGCCGCAAGAAGGGGGCCAA
GCTGACTCCTGAGGAGGAAGAGATTTTAAAGAAGAAACGATCAAAGAAAATTCAGAAGAAATACGAT
GAGAGGAAAAAGAACGCGAAAATCAGCAGTCTTCTAGAGGAGCAGTTCCAGCAGGGCAAGCTTCTTG
CATGCATCGGTTCAAGACCAGGCCAGTGTGGCCCGAGCAGACGGCTATGTGCTAGAGGGAAAAGAGC
TCGAGTTCTATCTGAGGAAGATCAAGGCCCGGAAAGGCAAATAAACCCTCTTCTCTCCTGTCTTAGCTC
ATCTAATAAAGGCGTTTACTGTTCTATGCCAAAAAAAAAAAAAAAAAATTNGGGGGGGGGGCGGGNC
CCCATTTCNCCNTTTAGGGGGGNNNTANNANNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNN
NNNGCCNGCCNTTTNNNTCNNNNNNNNNNNNNGNNCNNNNNCCNNNCCANNCNCTTNTTTNCTNTTC
CNCTCNCCNNGGGNNTATGATNNTGCTNACNNNTTCAGCCTNTATGATCGTATACAATGGCGGCCGCT
CTANAACTAGTGGATCCCCCGGGCTGCAGGNAATTCGGCACGAGGCATACNTTTTAACCCAAGCCNCG
ACNTTCTTAGCTGNGCCTCAGCTCTCACGGCCTCTNGCTCACCCATGAATGGACCCGCTTTCCTCTTGC
CGAGTGCACCTCCACCCATTGTCTTGGTGGCACATGGGTGGAACACTTGATCTGCTCGAGTCTGCCTTC
AACACACATTGCATCTTCAGATTTTCTACTTTNCTGTTTCAAAACTAACATTCNCCAAGTCAGACTTTGT
GNNAGNTCTATNTCNCGGTCTNCGGCGTGCCATGNGNTTCATCCCTANNTNGTGCTGAANATGGACNC
ATGGGAACTTNACANGCNCNTGNNTGNTNGGCACANGANTGCTTTTAGNGGCTANNAGNGNTCNATN
TGCNTGGNTAGCATNNGCTTGCGTGAATNCCTCCANCCCCCGANACNTGCTTTATANCTGATTTNTTTN
ANGGGNCNNNGGCCNCCTNNGANCTAGTNNATAANNCGNTNNCCGNACTGTCTNGNNNTACCNNACC
NTTNCNNTCTTTNNNTCNNGTTCTTNAATATCCNNNGCCCCTNNCANCCATTTTCCNNTTACNTTANCN
NTCTGCTATNCGCNCNNCTTNTNTCTCTTNGCTCTCCACCCCTANNTTCTNCCNTNCCAGCTNNTNCTA
TTTATCTTNTNGTNTNCTNTCNNTNCCGCTCNCCGTAATTNNNACANNCCACTTTCTCGCCCNTAGNTN
ACTATANCATACCCCTTTCCTNCNTNGACTCTGNNTTCCCCCNTTCNATNTCNTGCCTATTNNTTCNTCG
NCATNCGCTTTCGGACGNATCGTATNNTNTTCNTNTCATTCTCTCAACCTNTANNCGGACCTGTTNCNCN
ATTNGNNNTTNCGNANTNNTCCTNGTNTCTCTCTCTCCTACTTCTANNGGCNTNTTCNCCTCACTATTN
NTCTCTCTCNGCNNCGCNTGGNAGANATTCCTGACNTNTGNGCTCATTTATNCCNTCNGCCGCTNGGG
NNTCNANCTNACTANTCTCANACCGNTTCACTTNCCTANNTNTTNTTNATNTCGGTCGGNGTNTCTNTN
TANTNACCTNNANNACNANATGTCCGCGCGTCACGNNNNGTATCTCCNTCTCTTCTCGTNTGATCTCNT
CNGNATGNGTTGTCNNTCNTTGTCAGCTTACTCCTTCTTTCTCCC
CCCCCCCCCNNCTNNNTANNTANTTNTTTTTTTTNNNTNCTNNCNNNNTCANCTCTNAGNCGNTCTNN
CTNCNCGNNNNTCCTNTTTCNNATNNNGGNCGTGNNNTTGTNTCCCCANTTTNNTTTTGAGCCCNATAT
GAGTCGTATTACANTGNANGCCGTTNTANACCTGGCGGNTNCCCCTGGGCTCGCTGNAATNCNNGNNC
GAGGCTNNCTTTCTCNTTNNNACTCANNNCATNTNTTNTCTGNGNACCTCAANNCNNCCNGCCTCTTAT
TNNCCCNAGNNTNTGNNGTGCTTCTCTCTTNGTTGTNNTGCGCNCGTCNNGCCGTNTTCCCCTNNCNCT
CCTCNTTGGTCNGCTNACTAGANNNANCNTTNTNTNNCTATGCNCTNCNCTNCNTNCNNCNGNNCNTG
TTTCTCCCCNGTNCNCCTTTCNGNNTATNNTTNTNTCTCCTTANTNTCTGNTACTCNCNACNCCCCCCN
GAGNNNTCCNCCCCTNCNCANTNTCTNNTANNTTCCTGNTGTCNNCAACCTTTCCNCCTCTTTCCCACN
AGTTCCNNTTCTNATCTCTTNNTNNATTCGTCCTTNCTCTACNNTTTCNTNACNTGTCACTCTTCANTCT
NCNCGCTTCTTTTNTCNTNTCCCATCCNNANAGCGCTTTTAGCNGTNNTNNTCCTCANCTATNTCNTCGC
TCGCCTCTCATGCTTCCTTNGTCNNCTCNCCNTNCNNACCNCCNCNNACNCCTNANTTTCNCTNCCTCN
CNNTGCNNTTTNATNNTCTNCNTCNTCNTATTCCTCTCCTTNACNTCNTNAGNNNNTCNCCNNTCNCTGT
CACCNCTTCCCTCTNCNCCNTCCNTTNCTNCTTNCTNATCTTCTTCNTNCCTGACGCTCCTCGNTGTCNN
CTTTNACCCNTNGGTCCCTCTCATGTCNCNNCCANCCNNCCCCANNTCCNTNCCNTGAGTCTCTNTCCC
CTCNNCCCTGTGATCNCNNTCCTANNNTCCNTCNCNACCNCNTNNACTNCNNCCNNGNCCTNCANCTT
ANNNNNNTGTNNCTCACTCNNNTCNTTTGACCCACTANNCGTNNCCTCTTCTCCTCCNCTNTCGNTTAC
NTTTCNNATTATTTCCTGNCCNACAACNNNCNTNCGGNTCNTTGNGCTTACNNGNGCCTNCCACCCCT
NTANGTNNNGCTGCTCNNTTTTANCCNCTTATCTATTCCTCCNNTNCTCACCTATNNGNGCNCNTCTCN
GATCTNCNAGTGCTCTCCNTCGTCNNNGCCCCTTTAAGTGTTNTCTCCTNTTNNNTCTCCACTGNCTCGT
CTNNGTCTCCTNCGTCNTNTAANGTANTNCCTCNTGNGTCCTNNTNTATCGTACCCATGNTCTCNCNGG
GNNACNNCCTTACTTTACNCCATCGCGNCTAATCTCNTCTNCCNTCTNTTCGNNCCNGATNGNCACTC
NTCTTCTGTGCTAANNNCAGCGCNNGNTACNCANANTNGTGNTNTCTNCTCTCTNNTCCTACNCTTCNC
CGNNCNCACNNCATNAATCGTCGTTCCNTNNNNNTCCNGCAGNTGAACNNATCTCTNNTTACCC
CTCGCTCNNAGAAANGNGGGGCGCNCGNANCGCTNCACGNCCNCTNCTAACNCNTACTNNTAGCCCC
NTATCAGATGCAGGNTCGTCATTTACAGNGGCCNCNCNNCCCTANCGCACGNNANNTGNTATCCNNTCN
TNNCNNTNNACNAGTNTNNAGGGTCCATNTNTTNTCTTTTATTCNGTACTTNTATCTAANCCCCANCCT
ACTNGAANGNGGGCCCCTACNNNNCCGNANNNNNCNCACCGTNNCNANCTGNTTTNANACNCCNNNN
NNCTNTATGTTNAACNNNGNCNTNTNANNNNCNANNNTANTNGTNNGNNAATTNNNNNATTAGTGNA
CCCANNNNNNGCANNNTACNAATANTTTNNNTNNNNNNNGNNANTTCNNNGGCNCCCANCNNNNTTN
TTNNTNTNNNCANCNTNNNGGGNANGAANNGNTGNANTNTNTTNTNNCTAGANTNCNNGCNNCNTCC
TNGAATATTNNNNNNCATGNNNNNNGGNCCACNNNCATCATATGNNTNNCNNANGGNCTANTCATT
CNNCTCNTGNNNANNNTGATNGANTTNNNTNANTNCGATTCGGNNGGNNAGANTNCCATTNTNNTAN
ACGGNNNACGTNNGTCGNCNGNACNTTNTNNTANTTNNANANNGTNTNGCGNGGGNNCTGNCATATN
ANTTTCTTNACTGATTGGTCNTNGCCNTGNNNAACNACTGCNTNCTGNANTNNNNCANNNNCNTTGNC
GNGTATCANACTTCNTCNGNNATCNNNNNGATCNNNAACNTGTATTCAATTTNNNNNTCANTNCNANG
ATAGNTNNCTTCTTANGCNNNCNNAGCNNTCNNNGNNGTGNCNGCTNTCNGGGNNGNNATGTANANGNN
NTGNNCNCATNNGANGAGACCNAGTTCTNNTCTCNTCNACCACTCATNCNTTNCNATTNTCNTCTTCCC
NTCGTCNNNATCTCNCANTNNATNTACCAATNCNAGACCTCNTNNNCACTTTTTCCCAACNGACNANAA
TTGNTTCTTGTTCNATANAANCTNTTNTAANCNAATCGAANANCACNNNNNGTNANTNTGTTCAGTNN
TTCATCTCATATAGNAATNTCANNNTNANNCTTTNTNTGCTGCNNTCTANTGATNACGATCNACTTNNC
TGATNTTACTNGCTGANNAGTTGTAAGNGNNACAACNCTGACTNTANTACTCTNGNNATGGANTNNNT
NGTNCNCAGANTTGTAGNTAGCGCCTCNGTGNNAANACTNGNCTATNANAGANANTGNAGACAANNC
NAATGTAGANNGTGNNAACNCTNCTGACNACTNNACTTTACGTCANTTNCTGATNCTNTNANTGNCGT
NTCACTGTAGANATGTANNGTGNNNAGAGTNGATACNTATCTCAGANANGATANTNNGNGAGNNACC
NNTNTNATAGAATAGTGTNCTGTNCNATNATANTNTNCAGTCTTGTTNCANANCGTGNCG
TTNANCAANNCTGGAGCTCCACCGCGGTGGCGGCCGCTCTAGAACTAGTGGATCCCCCGGGCTGCAGG
AATTCGGCACGAGGTATAATCAAAAGTGGGTTAAAAAAAAGCTGGGTTAGGGCGNGNGTNCNCCTTG
AAAANGTTCTNTTNANCANTGGGGNCNTGNGTNTCNGGGGNAGNACCTTTTTTTTNCCNTANGNNNCC
CCANTAANNGTTNGTNNTTAAAAAAAAAAAAAAAAAAACNNGGGGGGGGGCCCNGGCCCCNATTCCC
CCTNATGGGNNGCNNNTTANNANNNNNNNNNNNNNNNNNNNNNNNNNNNNNNNNNNNNNNNNNNN
NNNNNNNNNNNCNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNC
NNNNNNNNNNNNNNNNNNNNNNNNNNNNNNNNNNNNNNNNNNNNCNNNNNNCNNNNNNNNNNNN
NNNNNNNNNNNNNNNNNNNNCNNNNNNNNNNNNCNNNNNNNNNNNNNNNNNNNNNNNNCNNNNNN
NNNNNNNTNNNNNNNNNNNNNNNNNNNNNNNNNNNNNNNNNNNNNNNCNNNNNNNNNNNNNNNNN
NNNNCNNNNNNNCNCNNNNNNNNNNNNNNNNNNNNNNNNNNNNNNNNNNNNNCNNNNNNNNNNNT
NNNNNNNNNNNNNNNNCNNCNNNNNNNNNNNNNNNNNNNNCNCNNNNNNNNNNNNNNNNCNNCNN
NNNNNNNCCCNNNNNNNNNNNNNNNNNNNNNNNNNNNCNNNNNCNNNNNNNNNNNNNNNNNNNNN
NNNNNNNNNNCNNNNNNNNNNNNNNNNNNNNNNNNNNNNNCNNNNNNNNNNNNNNCCN
TTNANCAANNCTGGAGCTCCACCGCGGTGGCGGCCGCTCTAGAACTAGTGGATCCCCCGGGCTGCAGG
AATTCGGCACGAGGTATAATCAAAAGTGGGTTAAAAAAAAGCTGGGTTAGGGCGNGNGTNCNCCTTG
AAAANGTTCTNTTNANCANTGGGGNCNTGNGTNTCNGGGGNAGNACCTTTTTTTTNCCNTANGNNNCC
CCANTAANNGTTNGTNNTTAAAAAAAAAAAAAAAAAAACNNGGGGGGGGGCCCNGGCCGCNATTCCC
CCTNATGGGNNGCNNNTTANNANNNNNNNNNNNNNNNNNNNNNNNNNNNNNNNNNNNNNNNNNNN
NNNNNNNNNNNCNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNC
NNNNNNNNNNNNNNNNNNNNNNNNNNNNNNNNNNNNNNNNNNNNCNNNNNNCNNNNNNNNNNNN
NNNNNNNNNNNNNNNNNNNNCNNNNNNNNNNNNCNNNNNNNNNNNNNNNNNNNNNNNNCNNNNNN
NNNNNNNTNNNNNNNNNNNNNNNNNNNNNNNNNNNNNNNNNNNNNNNCNNNNNNNNNNNNNNNNN
NNNNCNNNNNNNCNCNNNNNNNNNNNNNNNNNNNNNNNNNNNNNNNNNNNNNCNNNNNNNNNNNT
NNNNNNNNNNNNNNNNCNNGNNNNNNNNNNNNNNNNNNNNCNCNNNNNNNNNNNNNNNNCNNCNN
NNNNNNNCCCNNNNNNNNNNNNNNNNNNNNNNNNNNNCNNNNNCNNNNNNNNNNNNNNNNNNNNN
NNNNNNNNNNCNNNNNNNNNNNNNNNNNNNNNNNNNNNNNCNNNNNNNNNNNNNNCCN
CAAAAGCTGGAGCTCCACCGCGGTGGCGGCCGCTCTAGAACTAGTGGATCGCCCGGGCTGCAGGCTG
GTGATTCTTAATGATTTGAAGTCCATTGCTTGGGAGGAAATTCCAGCACACATTCATGCTTATAATAAG
CTGGGGATTTTTGTTTGTTTTTGCAAATGCTTGCCCCTACTTTGCAACGATTTTCTGTTCGTAATTCTGA
AGAACCAAGGTGGGGAGCAACACAACTGATGGAATAGTGGTATAAGTTTTATACCAGAAGCTTAATGA
CAAGTTTTATCAATCAGAATAATACTTGGTTATGGAAGTGACTGATGCTAAATAAATTCTGAGTATTTT
TTATTAGATAATTTCTCACATATAGACTTAAACTGTCAGCTTTGGTAGTGTCTATTAGTTAAACTTGTA
AAATATATATATATTCTTGTTTTTCCATTGGATGCAAATTGAAAGAAAAAGATGTACCATTTCTCTGTT
GTATGTTGGATTATGTAGGAAATGTTTGTGAACAATTCAAAAAAAAGATGAAAAAAATTCCTGNGGA
TGTTTTGNGTAGTATCCTTGGCATTTGNATTAATAGTTAAAGTTCACTTCCAAATAAAACTCCCATAAT
GCTAAAAAAAAAAAAAAAAAAACCTNGGGGGGGGCCCCGGNCCCCAATTCCCCCTNTTGGGGGGGNT
TNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNC
TTGANCACNGCTGGAGCTCCACCGCGGTGGCGGCCGCTCTAGAACTAGTGGACCCCCGGGCTGCAGGA
ATTCGGCACGAGGGACCGTTATTGCTCTCTTCCTATATTGTAAAAGTGCTCTTTTTTAAGGTAAGCATC
AGCCTTGCTGTCTGTAGATCTTCTGAGCAACACTATGTACACTGATACTTGGTTCATTTAGCTCTAGCA
GTACAATGATTAGTAATGTAAAAGTTANCCTAAGGAAGATAGCATGGATTTGTCAACATATCAAGTAG
AGTTTTGTTTATAAAGCATATTTGATAATCTAAAGAGTGCAGTATAAATCCTATATTAAAAATAAGATT
AAACAGGTTATTATCCATCCCATTTACTGGTAGACTTCAGGCAANGGNATGGTCACTCCTTTTGGACT
AAATTATAGTTGNGATGGGGACTTCATATTTACTGCAGTTTTGCCTAATCTCAATCATTGCATTTCCTTC
AATTAAATTTTTCTGAAGCTACATTGAGGAATAGCACTCTGCATGGTTTGGTTCTTAAACTGAAGTCCT
AAACCAGGAATTTTTTGGGNGGGAACCANGGAAGATNAACCTTTGGTGNAAATNAAAGGTTGCTANGG
ANTTATTTCNCTTTAAAAAANAAAAAAAAGGCCTTTNTGGGGGCCNACCTNCCAANAACTGGGTNGGGC
CATAAGAAAAGGGTTTTGGGCANTTTTAAACCCTAACCTTGGTTNAANAAAGACCANTTTCTCCATAA
GGCCNGGTTTTTGGNAAATTTTAAAGGATATTTTCTTTTAAAAAAAGCCAANGGTTTTTTTTCCTNAGG
AAATGCCCAGCCATTTAAATAAAAACCCCTGGGCTTTTATTGGGNAAAAACATGCCTTTTGGAANGNC
CCCNGGGGCTTGGGGGCCTTTGAANAACCTGGCCCCNAAAACCCNGGGGGCTGGAAACCCCGGGAAC
CAAAAGGGTCCCCCTTGC
TNNNNAAAAGCTGGAGCTCCACCGCGGTGGCGGCCGCTCTAGAACTAGTGGATCCCCCGGGCTGCAG
GAATTCGGCACGAGGCAAAATCAAGACCGATGAGATTATGACCTCACTCAAATCAGTCAATGGAGAA
ATAGAAAGCCTCATTAGTCCTGATGGTTCCCGTAAAAACCCTGCACGGAACTGCAGGGACCTGAAATT
CTGCCATCCTGAACTCCAGAGTGGAGAATATTGGGGTTGATCCTAACCAAGGTTGCAAATTGGATGCTA
TTAAAGTCTACTGTAACATGGAAACTGGGGGAAACGTGCATAAGTGCCAGTCCTTTTGACTATCCCAC
AGAAAAACTGGTGGACAGATTCTGGTGCTGAGAAGAAACATGTTTGGTTTGGAGAATCCATGGAGGG
TGGTTTTCAGTTTAGCTATGGCAATCCTGAACTTCCCGAANACCTNCTNGATGNCCAGCTNGCATTTGT
TCGACTTNTNTTCAGNCNGGCCTTTNAAAACATTACATATNACTGNAAGAATAGCATTTGCATACATGG
ATCATGCCAGNGGGAATGTAAAGAAAGCCTTGAGCTGATGGGGTCAAATGAAGGTGAATTTCAAGGCT
GAAGGAAATAGCAAATTCACATACNCAGTTCTNGANGATGGGTGCNCAAAACACCCTTGGGGGAAGG
GGNNAAAAAAGGNTTTTCCGTNTTNAAAACCCCCANGGGCGGCANAAATACCTNTTGGGGAANNTTT
GGNCCCCCTTTTTGAAATTTNGGGGGGCCCTGGAAAAAAAAAATTTGGGGGNGGGCCANTTGGGNCCC
TTTTTTTGNTTTTTTTTTNAANCCCAAANNTTTTTTTCCAAAGNTCCCCCNAAAAAAGGCTNCCCCCCCC
CCNCNTNNNCCCCNTTTTGGGTTTTAAAACTTGGGCNNNCCCCNTNCCAAGGGGGNCCNAANTTNCCNG
NTTTTTTNTTTNCCNAAATTTTTTNG
AAANCTGGAGCTCCCCGCGGTGGCGGCCGCTCTAGAACTAGTGGATCCCCCGGGCTGCAGGAATTCGG
CACGAGGATTAGGTCTTATAGGACTGGAGGGCAGACAGATGTTTTAATATAGTCTTTCAGCCCCTGCC
ACACTCANAGGTGGAGGAGTCACATGTGTCTTATACCGTAATGGGTCCCTGGCACCTGGCACAGACCC
AGGGAGCTCATTACATACTTATGGGAATGACTGANTGAAGGAGAGAGACCAGGAAGGAAATAGTTTA
TATTTTACATTATTAGATTATCACTGANATTTAACCTTTTCACAAATTANATGTTTAGAGTTGCAATC
TAGGCATTTTATTCCTACTCAAGCAGCTGAAAGATTAAGGATAATCCTTTTACAGATATATGTTTGACAC
GTCTCTGTGAATATGAATTAACTATGTAAATCTTGATTGCCTTTCGTGGGTGCTCAGTTCTCTCTAGAA
AAGCAGACTTANGTGGGCTGCTTCATGAAGCTTTAGGCAGCTTTCTTTCTCTCTTTTANGACACAGTG
AATTACCTTATGCTAAATGTAAATTGGCTTCCTAAANTCTGTTTACTTGCTGNGTCTCCCGGNNNNNNT
NTGAAATNNAANNTCNGGGGGGGGNCCGGGNCCCCATTGCCCCNTTTTGGGGGGNCNNNANANNNNN
NNNNNNGNNNANNCCCNNNNNGNCCCCNTTNNTTTNGGNNNGGGGGGNNNAAAANNANNNTTTTTN
AAANNNNNCNNNNNCNNNNNGGNNTTNNNNNNNNNCCCCCNNNNNNNNNNNCCCNCNNNNNTNNCC
CCNNNNNNTNCNCNNNNCNNNNNNNNNNNNNNNNNNANCCCCNNNNNNNNNNNCCCCNTTNNAAAN
NAAATTGGGNTTTTTTNANNNNNNNCCC
GCCTATNNGGAGNNGNATNNCAGTGGCGGCCGCTCTAGAACTAGTGGATCCCCCGGGCTGCAGGGNT
GAAGTCCGNTGATTAATGCCAGATGTACAANACAGNGGTTCATATTTGNATGTACTGGGAGATGATCA
CAATTANCATTTGTCACCATACACAGAGAAGATTTTCTTGNNATGACTACTTTNANGATCCACTCTNTN
ANCAACTTTCATATAAGACATCTGNGTTACAGTAACACACTGCCAGGTGGTCACTGACGTGAGGTGTN
CCTCATGTAACACCAATCGCCCANCCTNTTTATCAGGTTTACATATTGGCTCTGATGGATGTGANAGCTG
GACCATAAAGCTGAGCACCAAANAATTGATGCTTTTGAACTGTGGTGGAGAAGACTCTTGAGAGTCCC
TTANACTGCANGGAGATCANAGGAGTCNATCCTAGAGGAAATNAACACTANATATTCATCGGAAGGA
CTGATGCTGAAGCTGAAGCTCCAGTATTTNGGCCACCTGATATGAAGAGCTGACTCATTGGAAAANAC
CCTGATGCTGGGAAAGATTGAAGGCAGGAGGAGAAGGGGATGACAGATGATGATATGGCTGGATGGC
ATCCCGACTGAATGGATAGAAGTTTGAGTAAACTCTTGGGAGATGGACAGGGAAGCCTGGGATGCTG
CAATCCATGGGAGTGCAGGGTCAGACACGACTGACAACTGGACTGACTGGAGATTTGAGANGCCGAA
TGGGTGAGTTCTGGGGTGAAGCANTAACATCAACCTGTCCCTACTTGAGTTAAGTAGGGTCCTGGCTTT
TCCTAAATCAACCCANTGGGTTCCATATTGANGGANCCTGNTTAGTGGTGCTTAGTTTTCTGNCAAAGN
GCTNTTAGGAACATGCCTTTTTTTTGGGGNGNNAAAANAANAAAAAACNCATTTNTTGTTTTTAAAAA
ANNNNNNCTNNNNTNNNNNTCNNNNNNNNNNNNNNNNNNNCCT
TGAGNCCNANAGTGNNGTCGTATTACAANGGAGNCCGCTNTANAACTAGTGGATCCCCCGGGCTGCA
GGAATTGGGCCCGAGGCCNCNTTTGATAGNCTNCNNGNAGTGCCNAAANGATTGCGGGCACTNGTCNC
ANANNGANNNTNNGNAGANTTAGCTGGTGGAATNCGATGAACNGCGTCAGGANNNNGATCTNCTCAC
NCGNNNNNTATCNGGNNTATGAACTCGNNNNAANNNTNCCTTTATCTTNTTANNANTGCGTGACGNGNN
NNNTCNNTTNGATNTACTGCNNNGACCACNGNCANANGTNCTNTNCNGATNTNNGGANCNNCTCNCT
TTTNCNNNNNANNNNCANNNGNGCNNNNNNAGANANGNNGTGTGTNGGANNNNAGTTCATGAANGA
NNATNTNANCANTNTGTNNTCTTTATACTGNNTGTNANATGGNCATTAGTTTTNGTNNATCNCACACN
NANGNCTACANNANNGCGTCNANATNACANNCAAGTNNAGCTGNNNTNTGCTNNANATCNCTNTNAG
ANAANTGGCTNTNAACNGNTATNGNNCANTAGANNNCTGNATAATNNGNGNTGGANCNTTCACNATN
CNNTNCTANNNACNNGTNCTTNCATCCNNAATNGNNTAGNNTNNACCNTGNNNCNAACNCNGGTNCC
NGGAGNNCCANANAAANNCNNNNGGATGNACNTGANTTCNNGNTANAACNANTGTACNCCNNTATG
GACTNTNGTNCTNNNGTNCGCNNTGTNTNAANNCGGGNGNNCANAGCNNCNTNCTTACANTCNTTNNT
GTCNNTCTNAGACNCNGNTTTCCNTATGNTNACTNCATNACNTNTNNATTGTNGNNANNGGTNNCCCT
NGGNACCNTACNNNCTAAGNCATTTGNNNNTCANNANGNCCTTCTTTNNATCNGNANGATCNTTTNNCA
CGTGNTANNCTCNCANNGTNNATGACGAGGTCTANAGAACNACTTTNNGGCCCANNCGTATACCATC
ACTNNTNTNNTNGTANTNGGNNANNAANTCNTNNCTNCNCCCGNTNTTATCTANTATTCCNATTCNAC
TTTTGCACTTNTNCNCACNACGNTNTTGTNTNNATACNNAGCNANTCNNGGTNTNNTCTANNNCGTAC
NCNNGANTNACNTTAGANNCNNNTTNNNNNCCG
ANCANGAGGGGGNNGTTNNCCCGNCNCCCCCCCNCNCTNNGCCCTATAGNGAGTCGTATTNCAGGGG
CGGCCGCTCTAGAACTAGTGGATCCCCCGGGGTGCAGGCACNTAGCCTTNGAGGCNTNTCTNACGNTT
GGGCACTNATNCTGNCCCGCTNCGAGGGNNGCCAATTGTTAATGTNACANACTNCTCTCNCANNNTCC
CATCCTNCVTTTTNNCCCTNCNTNCTCGANCCCNNNNTCGCNNGTNTNNNGTNNGATCTTTCAGGANNA
CCACTGGANCNNCNGCNTTCTNNTNNANTTTNNATNCCTNNNAGGGTGGCNNANNNTNNCCNNTTTTTN
CNNCCNATNATNGCGNNCNNNTNNATNANTANNNTTGNTNNNCCTCCTNCNACGNNATNNNNNTTCTT
NTGNNNNCCCTNNGNNGCGTGTGNGNTNACCNNNNNANTGNNCNGNGCNTTTTNNTNTGNTTNAANT
NNNGNGGACNNGCTCCCTTTACCTNTTTNNTNTTTCTNTNGTGNNCTCCTNNNTNNNNTCNNNNGANT
ANNNNACANCNNATNNCNTATNTNTNNANCCTTNTCAGGTATTCTACCTNCTTNTCTACTANCTCCNTC
CCCCNGCNTTCTAAATNNTNTGNTTTCCNTNNACNTTNTNNTCTCTCNTTNTNNGCNCTGTANNNNNCTT
NCTCNNTCTCGNNCTNNTNCNNGNTTNTNNTANANTAATNNNNNNCTTTGTTCTCTNTCTNCCGTCNCN
NCNNTNCTCCNCCCATTNNTTTTTCCCCCCCCCCNACCNACTCTCCTTTATCCTGTTTTCNCTGCNCCTA
NTGTTTNAATNGNACCNCCTCTNCTNCCTTCCCCNTGNTNAGTCNNCATCNNCCTNTATTTNTNTTCTNT
TNTTTTGCTNNCTCTGATTTNCTANCNTATCCCTNNGTCTCNCNNTTTCATTNANCNNNNGAATNTCCTC
TATTAANNACATNNCGNTCTTTCNNNTCCNNANCTCNGAGTNGTCTATTNTNGCANCCCNTTNNTCATCT
TNTCACNNNTCNTNCANACTACTNTCNNANCAAACTCATTNTCNCCNGCCNTNNTNTCTTNCNNCANC
AGCNNTTNNNCNNGTCATTTCGTGCCTTGANANNNTCTAGTCTATANCTTTTCTNGTCTCGGTCTTGATT
CNCTTTCCTNNTTTGNTACTTNTTCTCNCTGTCACACTCATCTNNTNTNTNNTAGTGTGCGNCTGNNATN
ATNCTCNTCTTGNGACTNTCNNCNTNTATGTATANNNCN
GGGGGGGGTNCNNTNNCCGNNANTNTNNNTTNTNNNNTNNNNNNCNCCCCCTCNNTCNTCNNTTANN
NNATGGNNTGGNANNANTCNATNTNAGGGNGGNGNNCNNNGNCCNNCCCNTTTGAAACCNTNGGNCT
GCNNACANGGCGGCCGCTCTATAACTAGNGGATCCCCCGNNCTGCAGGGCTGNGNCACGAGNTAATT
CCGGTTGAGTGNTGGGTGTGCAGCTGCAGNGGGGACANNTGTATNCAAGTGAGNNCTGGATCCAACG
NTCNCGAGNTATTGNGNGTCCGTACNCTTANCCAGACNAGNCNNCAAGANCNCNTATTNCNTGNGCTT
CANAGGTGCTNCAAGGCCCGNNCGAGCCTGGCTCNTGGCNNTGGGGCAGNTGNNGAAANTTCNGGAT
NCCCCTATACNGGNTNACTCNGGANGGTGNNGNNNNNATCGGNGTTGCTGAANCTCTCANNCCGAGN
GTCCTACCTTCTNTTANCCTNTAAACCCGCTTCTCNNNGACTTTCTTNTNTNTTTNANNCTACCCCTTNG
TGNNTTGCCNGGANGNCNNAAATTANGAATTGGTNCAAGGCANGGCCNCCANNCNCAANNGNAGNNT
CNGAGACCGNNCCCCNGNTGGGNCNCCNCNNGNACACAANNNGNGTANCATGNAAANCCTNNGANA
ANANNGNTTGAGNNGCCTTGGCCNGGGANACNTCCCTTNNTNCANTATNANCNNTCTCNNNNANNTT
NNCNNNNAGNGAAACTGTCNNTCTNNNCCNCNCAAGNGGACNNATNNTGNNACANNNGGNAGGAGN
TGNAAGACNNNNTAGNANNNGTTGNNGNANNNCTAAGTNCNGCNNNANCTTGTGNCCTGTNNTNNNA
NGNTNNCCGTNTACTTTNNNNNNGGACGNCNCCGCNCCNNCTNNNTGCGNCNCTNTAGTATNCCTNGG
GGCNNGNTGTGCGAGGANTTCCNAANCAANATNNATNGAGANNNTNTTCCANNTNGNTNTTTACCTC
NNTAGTNGTNTNTCNTATNACGNNCCCNCGGANTATNNTTTGNNAGGTCNTNNNNATCCNAGGNNCTC
CACCCTGTGGNACCNCNTNNATNNNNNGNNCTCCAGNCAATGAGGNATANGGNTTNATCCCCTTNAN
ANATNTNTGTNTGANGNTNNGTGNCCNCTANNGATANAATGNCTCCTCTACNNCGATATCNANTNCAN
NGTNTGTACATNNTCNTNAAGGNACANATATTAGGCGCTTTNTACCATCTCNNACTANTTANNNGCCA
CTNGNGCGNNCTNTCTGCGNGCGCTCTCTATNATTGACTACGCGGNTCNGAGTNGNGNAAATCTCNAN
CGCTNCANTTACGGTAGNCGGNANANNGANCGAACNGNTCTNGGTAGGACGCGGGNTTGATGTANNG
AAANNTACACAGACNNTTGCGGGGGAANNTNTGNTCGNTCNTNTCNTACNAAACGCTNANNTGGCG
GGGGGGGGTNCNNTNNCCGNNANTNTNTNNNTTNTNNNNTNNNNNNCNCCCCCTCNNTCNTCNNTTANN
NNATGGNNTGGNANNANTCNATNTNAGGGNGGNGNNCNNNGNCCNNCCCNTTTTGAAACCNTNGGNCT
GCNNACANGGCGGCCGCTCTATAACTAGNGGATCCCCCGNNCTGCAGGGCTGNGNCACGAGNTAATT
CCGGTTGAGTGNTGGGTGTGCAGCTGCAGNGGGGACANNTGTATNCAAGTGAGNNCTGGATCCAACG
NTCNCGAGNTATTGNGNGTCCGTACNCTTANCCAGACNAGNCNNCAAGANCNCNTATTNCNTGNGCTT
CANAGGTGCTNCAAGGCCCGNNCGAGCCTGGCTCNTGGGNNTGGGGCAGNTGNNGAAANTTCNGGAT
NCCCCTATACNGGNTNACTCNGGANGGTGNNGNNNNNATCGGNGTTTGCTGAANCTCTCANNCCGAGN
GTCCTACCTTCTNTTANCCTNTAAACCCGCTTCTCNNNGACTTTCTTNTNTNTTTNANNCTACCCCTTNG
TGNNTTGCCNGGANGNCNNAAATTANGAATTGGTNCAAGGCANGGCCNCCANNCNCAANNGNAGNNT
CNGAGACCGNNCCCCNGNTGGGNCNCCNCNNGNACACAANNNGNGTANCATGNAAANCCTNNGANA
ANANNGNTTGAGNNGCCTTGGCCNGGGANACNTCCCTTNNTNCANTATNANCNNTCTCNNNNANNTT
NNCNNNNAGNGAAACTGTCNNTCTNNNCCNCNCAAGNGGACNNATNNTGNNACANNNGGNAGGAGN
TGNAAGACNNNNTAGNANNNCTTGNNGNANNNCTAAGTNCNGCNNNANCTTGTGNCCTGTNNTNNNA
NGNTNNCCGTNTACTTNNNNNNGGACGNCNCCGCNCCNNCTNNNTGCGNCNCTNTAGTATNCCTNGG
GGCNNGNTGTGCGAGGANTTCCNAANCAANATNNATNGAGANNNTNTTCCANNTNGNTNTTTACCTC
NNTAGTNGTNTNTCNTATNACGNNCCCNCGGANTATNNTTTGNNAGGTCNTNNNNATCCNAGGNNCTC
CACCCTGTGGNACCNCNTNNATNNNNNGNNCTCCAGNCAATGAGGNATANGGNTTNATCCCCTTNAN
ANATNTNTGTNTGANGNTNNGTGNCCNCTANNGATANAATGNCTCCTCTACNNCGATATCNANTNCAN
NGTNTGTACATNNTCNTNAAGGNACANATATTAGGCGCTTTNTACGATCTCNNACTANTTANNNGCCA
CTNGNGCGNNCTNTCTGCGNGCGCTCTCTATNATTTGACTACGCGGNTCNGAGTNGNGNAAATCTCNAN
CGCTNCANTTTACGGTAGNCGGNANANNGANCGAACNGNTCTNGGTAGGACGCGGGNTTGATGTANNG
AAANNTACACAGACNNTTGCGGGGGAANNTNTGNTCGNTCNTNTCNTAGNAAACGCTNANNTGGCG
NAAAAGCTGGAGCTCCACCGCGGTGGCGGCCGCTCTAGAACTAGTGGATCCGCCGGGCTGCAGGNTG
GTGATTCNAAGCNNNTNNNCANCCATAGCNCGGTGNGAACTCAGCGCGACANATTCGTGCTGTATATG
ANACTGGCAGNTCATTGAGGTGGAGAGAACAAGAGCTTGCCCCTACTTTGCGNCANAATATCNGNTCN
NNNTTCTNNNNAACCANNGTNNNNANCNNCNCNACNAATNCAATGTCAGGTNTTAATCCTNNNCTGA
AGGANTACTGNCATCANTTATTTATTACANTCATGAATCTGAAACGNTATTAAANGAAAAGCCTAATA
ACNACNCTTCTCTACAACNANAAATCACCNTNGGCCATAGCNAAAAACCNCACTGTATTTTGNCGGCT
GGAATCTTGGTTGGACATTCTCTNANTATTCTGACACCNCGNAATNCCCATTTTTTGNGGANTCTTTNA
GAAANTATTNTTNNTATGNACACATTCAAGCAGTTGGGNACCTTCCTTCCNCGCACCCTNAACACNC
ACAACNTTCATTCCCTTAGGAAGGCAAGACTTNANGGGAACCCTTTTNTTTAAAAAAAAGNACTAAGG
NCTCNTTTTGNATTACNAGGATTTGATNCCTGGCAAAAGTTNCCANCCGTAACNNNTTTGGNNGTTNNC
CCCCCCNCCNCCCCCCCGGNNCCCNNCAANCCNNGTCCCNTTTAAAGGGGGGGTNAAAANNATTTTNT
TTTTTNTTTTNTGGAAAAATTTTGNGGGGGCCCANATTTTAAGGTTTTTTTTGGNGAACCNNCTTTTNC
AAAANACCCGNGGCNTTAANAATTTTNGGGGGGGGGTTCCCNGGTTTTTTANNGGACCCCCTTTNTTN
GTTAANTTTNGGGGTTTTCNAAAAAAANAGGAAATTTTGGGAAGTTNCCAAAAANTGGAAGGGNGGAA
CCCCTTGGNAAGGGGTTTNCCCATTTCTAGGCCCNGGAAAAACNCTAAAACCCCGAAAGGGTNTANA
NCCNGGGTTNTTTGGTNCCCCCCCT
TTGANCACAGCTGGAGCTCCCCGCGGTGGCGGCCGCTCTAGAACTAGTGGATCCCCCGGGCTGCAGGT
CGAGGTCGAGCTTTCACTGCGGCAGCAGCAGAGCGGAGTGTACATTCTGATTTGCTACGTTTATATATA
TCTTGAAGCTAAATGTATATATGAGTAGTTTGCCATGAGATAACACAGTGTAAACAGTAGAGACCCAG
AAATTGTGACTTCTGTGTTCTCTCCATTTGAGTATTTTGTAATTTTTTTGAAATATTTGTGGACATAAAT
AAAAACAAGCTACACTAAACCAAAAAAAAAAAAAAAAAAAAAAAAAAAAAAAAAAAAAAAAAAAA
AAAAAAAAAAAAACNAAAAAAAAAAAANAAAAAAAAAAAAANNNNNNNNNTTNGGGGGGGGGGGG
NNCCCCCCCCCCCCCNNNTTTTNGGGGGGGGNNNNNNNNNANNNNNNNNNNNNNNNNNNNNNNNNN
NNNNNNNNNNNNNNNNNNNNNNNNNNNCCNNNNNNNCNNNNNNNNNNNNNNNNNNNNNNNNNCNN
NNNNNNNNNNNNNNNNNNNNNNNNNNNNNNNNTNNNNNNNNNNNNNNNNNNNNNNCNNNNTNNNN
NNNNNNNNNNNNNNNNNNNNNNNNNNNNNNNNNNNNNNNNNNNNNNNNNNNCNCTNNNNNNCNNN
NNNNNNNCNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNC
TTNANCAACAATCTGGAGCTCCACCGCGGTGGCGGCCGCTCTAGAACTAGTGGATCCCCCGGGCTGCA
GGTCTNGGNCCANCTTNNCTGCGNCNGCNGNGGANCGGAGTGNACATTCTGATTNGCTACNTTTATAT
ATATCNNGAACCTAAANGNATATATNACTGGCTTTGCCATGAGANNACACAGTGTGAANAGNAGACAC
CCAAAAATTGTGACTCCTGCGCCCTCTCCATTCACTGTNATGCCANANTNTTGANCTATTTNTGGACCT
AAATAAANCNNANCTACTCTTGTAACATGCAAAGAGGTTGGATCGAGTTTAAATGACTGTGCTACCCC
TTTTCACATCAAAAGAATGGACAACTACTGACAACGTAAGGCCGNGCCTGCCTCTCCCGTGGGGGNGG
GNCGGCTGCCANGGAAGGAAAAGAACTTGCATGTTTGGNGAANGANGAAACTTGGTGGGACCACAGTG
AAATTTAAAAGTAAAAAGCTGGGCCCANGNGGTCTGCNGGCTGNAAAAAGCCANTTTAATCAAAAGN
CCCTTTTTTTTTGGTGGTCAAAANGAATTTAAATTTTGGAATGGCNCACATTTTTTCCCCCCTTGCAAAT
AAAAAAGGTTTTTAAAACCTGGAAAAAAAAAAAAAAAAAAAAAAAAATTTGGGGGGGGGGGCCCGG
GNCCCCCAANTTCCCCTTTTNAAGGGGGGNNNNNNTNTNAANNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNCNNNNNNNNNNNNNNNNNNN
NNNNNNNNNNNNNNNNNNNNNNNNNNNNNNNNNNNNNNNNNCCNNNNNNNNNNNNNNNNNNNNN
NNNNNNCNNNNNNNNNNNNNNNNNNNNNNNNNNNNNNNNNNNNNNNNNNNNNNNNNNNNNNNNN
NNNNNNC
TTNNNCAAAGCTGGAGCTCCCCGCGGTGGCGGCCGCTCTAGAACTAGTGGATCGCCCGGGCTGCAGGA
ATTCGGCACGAGGATTACTTCACATCTGTCTTAGAGAATACTCAGTTACCACCCAAGATTTGTAGCATC
AATTAAGTTTTCTAATTGCTCACTGCTCTCCTTAGAACTTATTTGGTTTTAAGGTAGGGAGGAAAAACC
TTGCACACTTTGAAAAACAAACAGGATAGGACATGATCCTTGCTCCTAAGTACGGGCTTGAGGAATGTG
ACAGAGGAGTGCTTCATGTCTTTCGCTGTGGAGAAGTCAGTGGTAGTTTGTTGTATCCCTTGTTTGCCT
TATACTGGAGTTAGGTCTAGAACTGTGCCGTCCAATCATGTGTCTATAGAGGACTTGAAACGTGAGTA
GTCTGAATTGAGCCGTGAGGTAAAAGCAGAGAATACACTTCAGATTTCAAAGACTTACTGTGACAAAG
AGAACATAGAATACCTCAATAATAATTTCTTTTATGTTGATTATATGCTGACATAATATTTTGAATAAA
TTGGATTAAATAAAATATATTAAAGTTAAAAAAAATTCCTAAATTCTGTTTACTTGCTGNGNCTACANG
NNNNTNTNTGAAACCCTGNTCGGTTNCNGNNNNNNNNNCCCNNTNCCCCCTNNNGGGGGGCCCNNCC
NNNNNNNNNNNNNNNNNNNNNNCTTNNGNCCCCCTTNNNTTTNGGNNTGNGGGGNANNNNAANNAN
NNNNNNTNNNNNNNGNNCNNNNNCNNNNNNGNNNTNTNNNNNNNNCCNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNTTTNNAAACNAAATTGGGNTTTTTTNANNNNNNNNNNNN
GCCTATCTGGAGCTNCACCGCNGTGGCGGCCGCTCTAGAACTAGTGGATCCCCCGGGCTGCAGGAATT
CGGCACGAGGTTTTTTTAAAAAATTTTATTTAAATAATGTGTTTTAGTGTAAGATTACCCTTATAGGA
ATGGACAACGCTTCCAAGAGTATGAGCCTTAGATATTTCTCAAGAGTCAAAGCAAAGCAGCTTTTTA
AAAAATAAAAATTGGAAGCACAAGCAAGTGGCACTGGCTTAATGCTGTTAATGTTTCTAGAAGTTTCT
ACCTTGAGATTATATACCTGATTCATATTAAAATACCTCAATTAGCACTGTTTTATAGAGAACCTGAC
TTAATTGATTATTTTCGGATTTTACATGGGCCAGTTTATATGCTGTTATTCACGCTATTAGTTCGTGTAG
CCACGTGTTCTTTGTACCTTTTGTAGTTTGGTTTTACTGGGTTTACATCTTGATGGTCTAACACTCTAGT
TGGTTTCTGGGTGTTTCTCATGGGTTAGCATTTGTATATAGAATCTGGTCCATGTAAATGCTTTTCATGT
TTTTTTCCTCAAATGTTTAAACCACTAGTTGATGTATGGTATTTCTCTTCGGTTATTTGACTGTCCACTT
GCTCAACATATTGCTTCTAAAACAAACAATAAAGATTCTTTTATTTCTTAAGGAAAANNNNNNNNNNN
NNNNNNNNNNNNNCCNTTGGGGGGGGGGCCGGGGCCCAATTTCCCCNTTTANGGGGGGNNNNNTNAN
ANNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNC
TTNAGCGCTNNNNNGNANTCNNNNTNNNAGNGTATTACAATNAAAACNTCGTGGANNNTTNTGGGCT
GAAGGCAATNGCGGNATCAGGGANACNNNNNCCTCTNGTTNNTNAGTTNANTTATGACCAAATGGGC
ANCCTTATCTNNCATGTCANNATNNCACATNNGNATCAATTNCCATCGANANATCNATTANCGTNNCA
CNCANNCCTGGGCATGNGNTTNNTCGAGANCNNNACGACGNNNCCTTTCTAACNNGNNTGACNTAAT
GNNANTTNTCNTAACANNNNCCGANNAAGACCTATTTGCNGCNNNAGCNTCCAGNNGCANTTTTNTA
ANTTCTACNTTTCTAACNNATNNNNNNCCTCGTTAGNNTTCNCCGCNCTAACNTGNTAAGNCNTNTCG
ACCAGNNNGNATNAGGGNNNAGCNANNGGTNTNNTNCTNNANNNNNTATAATTANNNNCCTCTGTNT
ATTAACTTANANTANCGNNCNNAGTTNCCNNNAATTCNNGTCNNNNNGGGNTNTTANCNGTNNTCTTN
ATANATTGTNNCANCCTNNNNAGCNGCNTNANNCNNNNNNCTNNTGCCCATGGGNNACNGCANANAA
NTNAANTCNNANCCCNGNNCNATNAACANNANNACANGNNATNTNCNNCATGANGNNNNCATNNCG
ACNATAAATGANNNNNNCTANNGNNTTNCGTNNTTANANCATNNNTATACNTACANGCNNTNAGNAN
NNANTCGGAGTTGNCAGGNNATCTNANACNNNNACANGNNNANNTNGTATGACNNNTCNGTCTCNNT
AGACNNTANNNCANNTAGGCTCTNNGNANTCGNTANNAACAANNNGGANTTNTGNANTNTNNNACNA
TANTANNNNTGANGNANTACNTNNNTATACCANCCAANNNCNANTNTGANNNTCTNAGTNNCNNNGA
TGNTNANTNAANGACTNNTAACNTGNNNNATGATGAGTCNTCGGTCTNNTTACNCNGCCTANANNCN
ACTNNTNNCNNTCGTGTCNNNNTNACNCTTGNCNNNTGANATANNANNNCCGACGNAGTATGACGTT
NCATCNNNATACTTGTANACANACNCGANGANCTAGNANTANGTCTTNTNAGTNTNTCTTNACNNNTN
NTNCATNAANANTGNTANCGNGNCGCNNNATACGAACTCNNNCTCCTNACNNNTNNTNTATANGNAC
NNANNNGNTGGCGANGCNANNGTTAGNGTATATCNAAGTNTNTTNGNGNTNNNGNCNGNNTGGCAGN
GNCNCTAGNNGTANGNCTCTTGTCNNTGNACGCACGNANNTCG
GCCCTATAGNGAGTNGTATTACAGGTGGCGGCCGCTCTAGAACTAGTGGATCCCCCGGGCTGCAGGAA
TTCGGCACGAGGCTTGCTTCTCTAGGGAAGAATCCAACCGATGCGTACCTGGAAGCCATGATGAACGA
GGCTCCAGGGCCCATCAACTTTACCATGTTCCTCACCATGTTTGGGGAGAAGCTGAACGGCACAGACC
CAGAAGACGTCATCAGAAATGCTTTTGCCTGCTTTGATGAAGAAGCAACTGGCACCATTCAGGAGGAT
TACCTGAGAGAACTGCTGACCACAATGGGAGACCGGTTTACAGACGAGGAAGTGGACGAGCTGTACA
GAGAAGCACCTATTGACAAAAAGGGGAATTTCAATTACATCGAGTTCACTCGAATCCTTAAGCATGGA
GCGAAAGACAAAGATGACTGAAGGAACTTGAGCTAAAACTTTCCAGTTACTTTGTCTTACTCTATACT
GNTTCCTANACATTTCCCCCCACCCCCCATAGGCCTGTTGCATGCAACTTTGATTCACAGCTTTGCCTTT
TTTTTTTTTTTGGGNNTTTTTNTTTAANNCTTTCTNCCCNTNNNNCTTTTTTNTNTNANCCNNCCCNGNA
AAGGGGGGNTATANAANTTTTTATTTTTCNTTTGTAAAAANAAAGATTTNGNTANATTAAAANCCCNN
CNTNTTTGNNAACCCCNNNNNATTNTTTTATAAAAAAANAAAAAAANTTNANTTTNGGGGGGGGCCC
CCNCNCCCCCNTATTCCCCCTTTTTNGGGGGGNGCNCTTTNNAANCNNCCNGTNACTCTNTCTNTTTCC
ACCNCTTTGNNTTTNCTCTATCNCCCNTTTNNTTTTNNNTTCTGTCNCNCGTNTNCCNANTTCTTTCNC
CNTNTGCTCNTCTGANTTATAGTCTNATNTTTTCNTCTTCGCNTCTTGNTANNCNNNNTTCCNTTTCNTC
NTNNNNTNGCNTNTANTNTNATNCNTNNGNNCG
TTGAGGCCCATAGNGAGTCGTATTACAGTGGCGGCCGCTCTAGAACTAGTGGATCCCCCGGGCTGCAG
GNAATGGANNGCCNGGNCNGTCTAGNTGNNNTGTGNCTGNCCTCNGNCNGNGGGNNNGGNGGCGNA
CGNCNTGGCACCCTGGCCNTGNTNTATGCCNNCNTCGCTCANNNCNNNNNTNNCNNNTNATGNTTTT
ATAGGAACANTGANTGCTAATGGCNANTNCATATTGACATNGATNNNNCTGGGTNNAGNNANGACGT
TCCNANNNTNNTGANNNNGTTGGCATGCGTNNCNACCCNNAGACTGANNATTATGGANNATTATNAN
AACAAAGANNNGCACATTNTCCTTCCTAAAACAATTTCAGATGTCCANTTTGGTGAANTCNANGCGAN
GCTCAACGNCTCATNAGCGANANANTCACCTTATTNNAGACCTACTNTNNGAANGCGAANCNTNNNA
NTTATGTANNNGGTNNNNAGTGNAGNCNNNNTTGTANCANCNTAGNTTANTGTANNNGACCTGNNCC
NTTCAAGGNGTNATGGNTCGANTTCTGGGNNNCCCTGANNTNNNTCCNCTCNNTNNATTGGAAACAGC
CTTTNTTNTCTGGGNCAAATGNNTNNTAGCCTNATAAGCCCATNCCCTTGGNCTCTCTTGTTTTTCATN
NCTATGTAAACCTTTNTTTTTANNAGATTNTNNCATGNATGNCCCTTGCCTGNCTNGNTTGGCACTTNC
TNTTGTNTGTGGTTAAAATNTATTTCTCGAGANTACNTNGTCANTNNNNTGGCCTTNTTANGTNCTNN
AANAANNCCTTTTNTTNNTTCNTCNTTTATNTTNTNNTAANTNTTTNTCCNGANCTTNNCGAGGAGTNT
NNNGCTTTTTTGTCNACANATATNNCCACTCATGGACTNNTTTCTCNNACATACCNCCANNCNCANT
GTCTTTNTNAANANCATNNATTCNTCTTCTGANCTNGTANNGAAANCGGNGATGNTCNCTNCCTATTA
CTCTATNGTTGTTTCTGNNNNCCCCTCNNNGTTTNNCG
TTGAGGCCCATAGNGAGTCGTATTTACAGTGGCGGCCGCTCTAGAACTAGTGGATCCCCCGGGCTGCAG
GNAATGGANNGCCNGGNCNGTCTAGNTGNNNTGTGNCTGNCCTCNGNCNGNGGGNNNGGNGGCGNA
CGNCNTGGCACCCTGGCCNTGNTNTATGCCNNCNTCGCTCANNNCNNNNNTNNCNNNTNATGNTTTTT
ATAGGAACANTGANTGCTAATGGCNANTNCATATTGACATNGATNNNNCTGGGTNNAGNNANGACGT
TCCNANNNTNNTGANNNNGTTGGCATGCGTNNCNACCCNNAGACTGANNATTATGGANNATTATNAN
AACAAAGANNNGCACATTNTCCTTCCTAAAACATTTCAGATGTCCATTTGGTGAANTCNANGCGAN
GCTCAACGNCTCATNAGCGANANANTCACCTTANNAGACCTACTNTNNGAANGCGAANCNTNNNA
NTTATGTANNNGGTNNNNAGTGNAGNCNNNNTTGTANCANCNTAGNTTANTGTANNNGACCTGNNCC
NTTCAAGGNGTNATGGNTCGANTTCTGGGNNNCCCTGANNTNNNTCCNCTCNNTNNATTGGAAACAGC
CTTTNTTNTCTGGGNCAAATGNNTNNTAGCCTNATAAGCCCATNCCCTTGGNCTCTCTTGTTTTTCATN
NCTATGTAAACCTTTTNTTTTANNAGATTNTNNCATGNATGNCCCTTGCCTGNCTNGNTTGGCACTTNC
TNTTGTNTGTGGNTTAAAATNTATTTCTCGAGANTACNTNGTCANTNNNNTGGCCTTNTTANGTNCTNN
AANAANNCCTTTTNTTNNTTCNTCNTTTATNTTNTNNTAANTNTTTNTCCNGANCTTNNCGAGGAGTNT
NNNGCTTTTTTGTCNACANATATNNCCACTCATGGACTNNTTTCTCNNNACATACCNCCANNCNCANT
GTCTTTNTNAANANCATNNATTCNTCTTCTGANCTNGTANNGAAANCGGNGATGNTCNCTNCCTATTA
CTCTATNGTTGTTTCTGNNNNCCCCTCNNNGTTTNNCG
NAAAAGCTGGAGCTCCACCGCGGTGGCGGCCGCTCTAGAACTAGTGGATCCCCCGGGCTGCAGGTCG
AGGTCGAGCTTCACTGCGGCAGCAGCAGAGGGGAGTGTACATTCTGATTTGCTACGTTTATATATCT
TGAAGCTAAATGTATATATGAGTAGTTTGCCATGAGATAACACAGTGTAAACAGTAGACACCCAGAAA
TTGTNACTTCTGTGTTCTCTCCATTTGAGTATTTTGTAATTTTTTTGAAANATTNGGGGNCNTAAATAAA
ANCANNCTCCCCTAAACCAAAAAAAAAAAAAAAAAAAAAAAAAAAAAAANNNNTNNNTAAAAAAAAA
AAAAAAAAAAAAAAAAAAAACAAATAAAANAAAAAAAGAAAAAATNNNNNNNGGGGGGGGGGGN
CCCCCCCCCCNNCCNCCCNNNNGGGGGGGTNNAAAAANNNNNNNNNNNNNNNNNNNANNNNNANNN
NCCNTNNNNNNNNNNNNNNNNNNANNNNNNNNNANNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNC
GNANACAGCTGGAGCTCCACCGCGGTGGCGGCCGCTCTAGAACTAGTGGATCCCCCGGGCTGCAGGC
ACNAGNCGNNCTTCCTTNNGCTGGNAAAGTNCCNTGTNCGNACACTGAGTCCTGCTCTGTGTATNAAG
GANACATTGCCTCNGGCTGAATGAAGCAGGGTTTGTGAAAAGTGCTCAGAAACCACAAGTTCCCTGTG
CTTTCACAGNTTTTGAGGCTTTTCCTTTTCACACTGGAGACTTTCTTCTGTTTNAATGGGGCTACTGTG
TGGCTCANAAATGGCTGCTGTGATGAAAATGCTCATTTGTTAGGAAAAAGAGGTAGCTACNTTCTCAT
TTTGAAAGGACCATGAGCTATTTAAAAAAAAAACCNAANANGNGGTTTTATTTACNTGNCAGGGGAN
CNTTNGATCATGTTCACATGGTTTNAAAANAAANCANTTTTTTTTTCCTNCTTTTANGGGTAAAAACAA
AAAAATTTTTNTTTTTTNTNNTNAAAGNCCCTTNTAGGNAANAAAGGGATGCNTAACCCGAANGGTTA
GGNACCCCCAAGTTATTCCTNGTNAAAGACCCATAATTTTTTGNAACCNGGAANCGGGGAAAACAAA
AGTGCCTCCCNAAAATNTAANAAAAGCCCNTTACCTTCGGATTTGGNAAATNNAAAACCCTTCTTTTT
AAAAAAAACCCNTTTTTTTTTAAAAAAAAACCGGGGGGGGGCCTTAAAATTGGGTNAAANGCCCAAA
AAAAAAAATTTCCCGNGGGATTTTTTGGGANNAAAAACCATNNTTTAAAAACCTTTGAACCCCTGGAA
AATTCCTTNTGGCGCGGGGGGCAAAATTTCAAAAAACCTTTTTAAATCCCCAAAATTTTTTGGNGNGG
GGAAACCNTTTGTTTTTTCAAAAATTTTCNCCCAGGCNNGGGAAAACCCCCNGGGGTTTTNTNNAAA
AAAANANGGGNNCCCCCT
TTGAANACATNTGGAGCTCCACCGCGGTGGCGGCCGCTCTAGAACTAGTGGATCCCCCGGGCTGCAGG
TCTGCAACCATGTNTGTTTNNCCCGATATGGCTGANATTGACAAGNTCGNTAAGNTGAAATTGAAGAA
AACGGAAACGCAAGAGAAAAATCCACTGCCTTNGAAAGACACGATTGAACAGGANAAGCAAGCAGG
CGAGTCGTAATGAAGCGTGCGCCGCCAGTATGCACTGTACATTCCACANGCATTGCCTTCTTATTTTAC
TTCTTTTAGCTGTTTAACTTTGTAAGATGCAAAGAGGTTGGATCGAGTTTAAATGACTGTGCTACCCCT
TTTCACATCAAAGAATGGAGAACTACTGACTAACGTAGGCCGCGCCTGCCTCTTCCATCTGCTTGTGTGG
CTGGCAGGGAAGGAAAAGAACTTGCATGTTGGTGAAGGAAGAAGCTGGGTGGGACCACAGTNAAATC
TAAAGTAAAAAGCTGGGCCAAGGTGTTCTGCGGGCTGTAAAATGCAATTTAATCAGANTGCCATTTTT
TTTTGNTGGTCAAAAGANTTTAATTATTGGAATGCACAATTTTTTTNTTATGCAAATAAAAAGTTTTTA
AAACCTGAAAAAAAAAAAAAAAAAAAAANTTTNGGGGGGGGGGCCCNCGGANCCCAATTTCNCCC
NNNTAAGGGGGGGGNCNNNTNAAAANNNNNNNNNNNNNNNNNNNNNNNNNNNNNACNNGNNNNNCC
CNNNNNCCNNNNNNNCCCNNNNNNNCNNCCNNCCTNNNNNNNNNCNNNNNNNNCNNNNNNNTTNNN
NNNNNNNNNNNTNNNNNNNNCCCCCCCNCNNNNNNNNNNNNNNNNTNNNNNNNNNNNNNNNNNAN
NNNNCNNNNNNNNNNNNNNNNNNNNNNNNNNNNNNNNNNNNNNNNNNNNNNTNNTNNNNNNNNNN
NANNNNNNNNNNNNNNNNTNNNNNNNNNGNNNNNNNNNCNNNNNCN
NACAAAGCTGGAGCTCCCCGCGGTGGCGGCCGCTCTAGAACTAGTGGATCCCCCGGGCTGCAGGTCTG
CNACCATGNCTGACNNNCGNGNTATGGCTGAGATTGAGAAGTTNGATAAGNTGNAATTGAANAAAAC
GGNAACNNAAGAGAAAAATNCACTGNCTTCGAAAGAAACGANTGAACAGGAGAAGCAAGCAGGCGA
GTCGTAATGAAGCGTGCGCCGCCAGTATGCACTGTACATTCCACAAGCATTGCCTTCTTATTTTACTTC
TTTTAGCTGTTNAACTTTGTAAGATGCAAAGAGGTTGGATCGAGTTTAAATGACTGTGCTACCCCTTTT
CACATCAAAGAATGGAGAACTACTGACAACGTAGGCCGCGCCTGCCTCTCCCATCTGCTTGTGTGGCT
GGCAGGGAAGGAAAAGAACTTGCATGTTGGTGAAGGAGGAAGCTGGGTGGGACGACAGTGAAATCTA
GAGTAAAAAGCTGGTCCAAGGTGTTCTGCGGGCTGTAAAATGCAGTTTAATCAGAGTGCCATTTTTTTT
TGTTGTTCAAATGATTTTAATTATTGGAATGCACAATTTTTTTATTATGCAAATAAAAAGTTTTAAAAC
CTGAAAAAAAAAAAAAAAAAAAAAANGGGGGGGGGGGGCCCGNGNCCCAANTNNNCCTNAANNNG
GGNGGNTTTNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNGNNNNNNNNNNNNGNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNN
NAAGCNCCNTTNNNTGTNGGTCNNNNTNGNAAGGGGGNANTACCACTTTTAANAANNTAGGTNGGCA
ATCCCCCNGNTCTTGCTCTNCTNCTGCNCCCTCNACTAGTCTTTTTTTTCCCCCTTNCTNNGACCCAN
TNCCAATNNATTANNGNGGNTCNANTTTTAANATTNTTTNANCTATCCCTNAANNCNCCGNNCCNTNC
TNANCTTCTCNCTNNCTNNNNNNATCCANANNNCNANCATGNGAGGATGTAGNANNNGNNNNNANNG
AAANNACCNATAACTTTNCTTNTNCCCGNAGGGNANANTNGTGCTNNNGTNGAATGANCNANCNGN
NANNNNTNAGANNNNANNNCNACCCCGANTGCTNNNGGGNATNAGNTCNNCCNGGGATNCATNN
GNGTCNCGNAANNCTATGGAGNCNNAACNNNNAGANNTANNANNANNCNNNNNNNNCNCCNNGTAA
GCGAGNCATGTNNATNTCATNGNNTNCTTTAGACTCCAANANNGNNNCCAANACACNATTANANNC
CTACNTCNANNANCGNNNATNTNTGTNNGCNNNGNGTNNNNNGNGNGNNATAGGNATTATACNNNN
NATTNTGNTAGANACANAGGCTTGTGNNTANNNNNANNNNNNGNNTGTTCATNTNANTCGAANGANA
NTATNTNGCAGNANGNANNNNNGAGTNNNNNGNTNANTGATNANCCCNGNNCANTATACGGATTNA
NNNCTATCANANNCNCCNGNNNNTCANGNNNANTNCNGACNCNGGCNNCAAGNCCCCNGNNNNATT
NNCTNAAAANANCNAAGNNTCNNNANNGGANNGGGCGAANANTNNNNGCNNCNAGGTAGNGNNNCT
CNNNCNANATTTCAATGTCTGCANNGCCCNNCCNNGNGNACCAGTTGTTCANGANGTGCGNNCNGNC
NGCNNTANAANAANCTNTNCNNNCGTGNTNANTANGTGCAGGNCGNATGGTNNTNNNNNCGCATNTC
GGANANTNGNNGCNAACAGNNTCGCTGNNGCNCNCTGGTGANGGAGCAATCNNTCGNNTNACATA
NCNTNNNTNNANAGTANCCAAGAGCNGNNATTGTCTNNNNNGNCAGTANNANNTGCGTCANNTNNGC
NGNGNNNNTNTNCATNCGNTATATGTACNATGCNGAANNNCACCNCNCTNTCANNNCTGCNTCNNGT
TTNNNTTATCATAGCNANCGNCCNGCGANACNATCNAAANTANTGAGTNCNANNNCNAGCNATGNAT
CNATAACGAGTNTGNNANNAGANTGTNGNANNGCCNNANANCTATCGNGANAGNATANAGTGNTCG
NT
TTAANNCNTTTANNGGANNNNTTTANNNAGGGNATTACACTTNNACNCTTGTGGNTCCCCCGGGCTGC
AGGATCCNANNNTCCGNACCAGTTNTNNNGCTCGAGACATGAGTTNGGACCTGAGGGCACCGGGATG
ACNNTGNATTTNCTTTGCANNATNAGCCATGAATTTCTNTCCNGGGNATNNTNTNNAGCATGNNGAAN
NNTNNGTCTAACCGGGNATNNNTNNNCNNATNNTANTGTCNNNATNCNNGNNCANNNNNCTNNNGNN
NNNAANNANTNTCCTAAACTCTNNNTNCNNNTGTNNTGNAGNNNCNNCGAGGANTTATCAANNTGGN
NANCNAGTNTTCATTGNCNNTTTGCNANCNNNACTGNCNGTATNNNAGNNNNANATCCTNNAAATAA
NNGATCNANNACCANNCATANTCGTATNTTNNTNANNCCTNACNNAANCANNCCNNNTAATTCCNNN
NNNTCATNTGTNNNNCATATNGNCNACNNNNATCNNNTTNGANCNNNNGATNNTTCAACNTANNTCN
ATTGTNTTACTGAGNNAAGANNNGTTNNACNTNANTNTATNNAGNNAAAGNNNNCTCTGANNNNTGT
CNNTGTNNTGTNNGTTNAAANNNCTNCTTTNTNANCNCNTCNCTTANGANTCNTANGNTATTATTNCC
AACNTNTTTNCGGNTTCGTANANNTANNATTGGANCATNACTNTTCGTNGTAGANNNAANATCTACGT
ANNNAATTNANNTNNNGANCATAAANNCCCCGACCNCACGNANNNNNCNNCCNNGNNNGCANTGTG
ANCATANAATACTGANTNANCNNNTTANTTTNNNNNATAANNCNTNNTGCNANCACTANACCCNNNN
CNNGACCNCNAGGNATCTAATNATNNANTNNNGTGNGNNCTNNNGAANANTNNCNAGANNCTCNNTG
NNCAANTNCNNCNCNNATTACCNNCNNAAATCTTNNTCTNATGCNATTAAACNTNTGTGANGTAANN
NNTANCGTGTGCTGCTCNCNANNAGANNNCNANNNNTACNNCNATCTANATNAGAGATNAACNNTNT
NTACACTNCNTATANTCCTCCTNCNNAGGTAGAACATGANNTNTCCTNGNTACNCACCCNATGNNGAN
TTNGGCCGNNTANCGATCTNANAGCANGNAAGNANTAATAGCATTNACTNNCGACTNANTANNTAGT
CAACNANNCNCCCG
GCCCTATNTGGAGCTNCANCNCNGGTGGCGGCCGCTCTAGAACTAGTGGATCCCCCGGGCTGCAGGTT
CTACCACTGACTCAGGCCCCGATCTTCATCTCCTTCTTCATTGCCTTGAGAGAAATGGCCAACCTTCCC
GTGCCCAGCCTGCAGACAGGTGGTCTCTGGNGGTTCCAGGATCTCACACTATCTGACCCCATCTACGT
ATTACCTCTGGTGGTCACAGCTACAATGTGGGGTGTCCTTGAGTTANGTGCTGAAACTGGCATGCAAA
GTTCTGACCTTCAGTGGATGAGAAATTTTATCANACTAATGCCCCTGGCAGTCTTGCCCATAACTATCC
ATTTCCCCACGGCANTGTTNATGTACTGGCTCTCCTCCAACATGTTTTCCCTGGGCCAGGTGGNCTGTC
TCCGTATTCCTGCTGTACGCACTGGACTGAAAATTCCCCNACGTGTTGTGCACGACCCANACAAGNTA
NCTCCACNAGAAGGCTTNCTAAAGAGCTTAAAACAAGGCTGGANGAATGCCGAAATGGCCCATCAGC
TACAGGAACGGNNAACGACNTATGCAGAAACCACTTGGGAGCNCGCTGCCAGGGGTTCGCTACGACA
GACGTTTGCCCACAACNCCTCTGNNGCAGCATTGGAAAGAAATGACCCTCCCAACACCCCCTAACAGG
CAGCAGCANGCAGNGGGCNGNAGTAANCAAAAGNCANAAGTCNAAAGCAACCCCTGNCGTTGACAC
CGCTTTGGCTTGAANCCTCTGGTNGCCTTGNTTCACAACTNGGGNNAGGGAAACTTCNTTTTTTTTTTT
NANAAAACTTNANCCCTTTNAANAACCGAATNTNCATGGCNGGGGGNTNTTGGCGCCCAGAACCCTT
NAAAAAACTGGNGGGGGACATTACTTGATGGTTNCATNTTTAAAAAAATGGGANTGCTNCNTACAAG
AAANTCTTTCCNCCNTGGATNGGC
TTNAGCCCTATAGNGAGTCGTATTACAGTGGCGGCCGCTCTAGAACTAGTGGATCCCCCGGGCTNGNA
GGATTGAACGGNCTGCTCACCCCGGCCGAGTGTGGNGTGCTGCTGCANCTGGCCAANGATGCNGNTG
AAGCTGGAGCCAGGGCTGGCTATNGGGGTCATCGGTNCCCACACACCCCCCANGAACGCTTTGAGGG
GCTNACNGNGCTNAAGGCCGCACANCTGGCCCGGGCTGGGGCGGTGGGNANTCAGGGTGCCCATCTG
CTNTCTGGAGGTNANCAAGCGGGTGCGGAGCCTAACCCAAGGCCTANTTTCCCCCGACCGGGCGCTGC
ATNTCTCCTTAACCCACCTGNNGTGCCGGAGCGCCATATAAGGGGAGCAGGCGCACTCGCATGGACCT
GAGCCACCCGGTGCACGCCGACAACTGTGTGCTGGATCCTGACACCGGGGAGTGCTGGCGGGAGCCC
CCANNCTACACCTATNGAGACTACAGTGGACTCCTCTACTCANCGATGACTTCCAANGGCGGGGACCT
GTTCTTCANGGAGCCCAACGCCCTCACAGTAACGGCACAAGTTCNTCCTCGCTGCNGCCGNCTCGTG
GCTTCAGCTNAGGCGNGGAANAATNCCCACGGTGTTGTTGNGCCCGTNANNCCCCGGCCGGGNGCTT
GTNCCCCTGGCAACTTNNNGCAAAACCTTGNGNCCCCTTGANCACAAAGGNAGCCANGAAGTNGGAT
GGGAAGCCCANANNAAATTTGCNTGTNTNGGGCCCCNAAAAAAGGATGAAANGGANNAAAGATTGT
NNTCAGCCANGGGAACCCCNNNTNCAAAAAACCCCCCAACCCGAANGNCTTTCCNTGGGTTTCCAGG
AAAAAAGGTTNGGGAAANCCCCCCTNNCNTTTTCAAAAGGAAAATTTTTNAAGGGGGCNTGGCCCCA
AAAAACTTTTGGGGGAATTTGGCNCNNTTNAATTTGNGGGNGGGG
TTNAGCCCTATAGNGAGTCGTATTACAGTGGCGGCCGGTCTAGAACTAGTGGATCCCCCGGGCTNGNA
GGATTGAACGGNCTGCTCACCCCGGCCGAGTGTGGNGTGCTGCTGCANCTGGCCAANGATGCNGNTG
AAGCTGGAGCCAGGGCTGGCTATNGGGGTCATCGGTNCCCACACACCCCCCANGAACGCTTTGAGGG
GCTNACNGNGCTNAAGGCCGCACANCTGGCCCGGGCTGGGGCGGTGGGNANTCAGGGTGCCCATCTG
CTNTCTGGAGGTNANCAAGCGGGTGCGGAGCCTAACCCAAGGCCTANTTTCCCCCGACCGGGCCCTGC
ATNTCTCCTTAACCCACCTGNNGTGCCGGAGCGCCATATAAGGGGAGCAGGCGCACTCGCATGGACCT
GAGCCACCCGGTGCACGCCGACAACTGTGTGCTGGATCCTGACACCGGGGAGTGCTGGCGGGAGCCC
CCANNCTACACCTATNGAGACTACAGTGGACTCCTCTACTCANCGATGACTTCCAANGGCGGGGACCT
GTTCTTTCANGGAGCCCAACGCCCTCACAGTAACGGCACAGTTCNTCCTCGCTGCNGCCGNCTCGTG
GCTTCAGCTNAGGCGNGGAANAATNCCCACGGTGTTGTNGNGCCCGTNANNCCCCGGCCGGGNGCTT
GTNCCCCTGGCAACTTNNNGCAAAACCTTGNGNCCCCTTGANCACAAAGGNAGCCANGAAGTNGGAT
GGGAAGCCCANANNAAATTTGCNTGTNTNGGGCCCCNAAAAAAGGATGAAANGGANNAAAGATTGT
NNTCAGCCANGGGAACCCCNNNTNCAAAAAACCCCCCAACCCGAANGNCTTTCCNTGGGTTTCCAGG
AAAAAAGGTTNGGGAAANCCCCCCTNNCNTTTTCAAAAGGAAAATTTTTNAAGGGGGCNTGGCCCCA
AAAAACTTTTGGGGGAATTTGGCNCNNTTNAATTTGNGGGNGGGG
TNNNNAAAAGCTGGAGCTCCACCGCGGTGGCGGCCGCTCTAGAACTAGTGGATCCCCCGGGCTGCAG
GCGCCTGCCAAAGCAGGAGACACAGATTCGATCCCTTCATGGGGAAGATCCCCTGGAGAAGGAAATG
GCAACCCACTCCAGTATTCTTGTGTGGAGAATCCCATGGACAGAGGAGCCTGGCGGACTACAGTCCGT
GGGGTCACAAGAGTCAGATAGGACTTAACAACTAAACAAAAACAACCTATATTAGAAGGTTGCCACT
ATTTTTTCCAAGACAGAGCCAGTCACTCGCGGANCAACTTTNGATGTGGCCTGTGTGTCTAACCATTCN
TAAATGCCTGATGGCTAACNTGTGAAANCAGAANTTTCCCATTNGTTGGAAAAGCCTTTTGGCNCGGC
CTTTCCNATCTTATTAACCAGGCACCGTTGGTTGGAAAAAGNGAGGNCAGAAGCCAAAAGGGGCTTGT
GGTGGAAAGAATNGAAGCTTNATTTNACGCCCTTCTAACAANCTTTGGGGGAAGGGTTTAAGGGAACC
TTGGNAATATGNAGTGGCAATNGGNAANGGNAATNCCTTGGAATAATTGGGNCCCCCNTTCNTTTTTT
TCTTCCCCCCCAACCAAAAANACCNTTATTCCTTTTTTTAGGGANGCCCCCCCTTGGNAAANAGGAGG
GNGTCAAAAAATGTAAANCGGGAACCTTCTGGTTTCAAACCNAAAATTTTTCANTTTAANTTTTNATTN
GGCCNTTNGNGGAANCCNAAAAATTTGCTTGCCTGGNCCTGGGAATGCTTTTTNCCAGNNNAAAGCNT
TTCNTTTTAATTTTTTTNAAACNCCCTTNCCTTAANGGCNGGGGGGGGGCCNCCCCCCAACCCNTTTT
TTTACCCTTGCCCTTTTGGGGGNAACAGGNAACCAAAAACCCCCCTTANGGGGTTGGNNTTTTGGTTT
TACAAAAACTNGGGTTGGA
TTTNANCAAAGCTGGAGCTCCCCGCGGTGGCGGCCGCTCTAGAACTAGTGGATCCCCCGGGCTGCAGGC
GCCTGCCAAAGCNGGAGACACAGATTCGATCCCTTCATGGGGAAGATCCCCTGGAGAAGGAAATGGC
AACCCACTCCAGTATTCTTGTCTGGAGAATCCCATGGACAGAGGAGCCTGGCGGACTACAGTCCGTGG
GGTCACAAGAGTCAGATAGGACTTAACAACTAAACAAAANCAACCTATATTAGAAGGTTGCCACTATT
TCTTCCAAGACAGAGCCAGTCACTCGCGGAGCAACTTTTGATGTGGCCTGTGTGTCTCACCACTCCTAG
ATGCCTGAGTGCTTACATGTGAAAGCAGCAGCTTCCCACTTGCTGGGAGAGCCTTCTGGCACGGCCCT
TCCGATCCTACTAAACAGGGCACGTGGGTGGGAAGAGTGAGGACAGGAGGCAAAAGGGGGCTGGTGT
GGNAAGGANTGGAGGCTGATCTCAACGCACTCCTANCAGCCTNTGNGGAAGGGTCTCAAGTGACCCTT
GANATAGTGAGTTGCAATGTGAAANGGAAAGCCTTGGGATCATTTGGGCCCCCCTTTNTTTTTTTTCTT
CNCCCCCCCCAAATACACTTTATTCNTTTTTTTGGGGGAGCCCCCCGGGGGGAAANAAGGNGGNGTTT
CCAAAATTGTNANGNGGGGAACNTCNNTGGTTTNAAAAAAAAAAAATTTTNATTTCTTTTTTTNNGGG
NNNNNNGANGGAAAAANAAAAAATTTGNTTGNCTGGGCCTNGGGATTGTTTTTTCAGGGNNAAAGGN
TTTCCTTTTTAAATTTTTTTTAAACNCCCTTCCCTTAAAGGGGGGGGGGGGGGGGGGGTTTTANNNAAAA
CCNTTTTTTTACCCATGGCCTTTTNGGGGGAAANAGGGAAAAAAAAAANCCCCCCCNNGGGGGGTGG
NTTTTTTTTNNTTTTCCCAACNG
ANACATNTGGAGCTCCACCGCGGGGGCGGGCGCTCTAGAACTAGTGGATCCCCCGGGCTGCAGGGTTT
CCAGGACAGCTGATGGGAGAGGAAGCTGGGTGTCTCCCAAGAGTCCCAGGCTGCAAGGGGAGAGAG
CTGACCCTTCTCAAACCCCTGCTTTGGGCCCAGCCCTGCGCCAGGTGCTTCACAGCTCTTATCTGCCAG
ATTTTCCCACCTTGTGGCACATGGCGCCCCTAACCAACATCCCAAGAGGCCGATCACCTTGCCACATCT
GTGCAAGACCTGGGACCACAGGCGTGGACAGGAAGGGCTCCGAAGCCCTCTTTTTACAGATGAAGAT
GCCAGGGCCCAGGGAGGATGAGCAGTCTTTGGGGTTACCCAGCCGGAATACTGGGAGACCTGAACCC
AGCATTCTGACTCTGCATTCAGTGCTCGCTCCATGATGCAGCGGTCCTAGCCCCCCCCTCAAATGGGTC
CCCTCTTCACTGTGTTTCCCAGCATTCCTTGCTCTGACCAAACCGGGGAAGGGACACAGCCCACCCCCA
AAAGATGGAATCATTCTCTTAACCGTCTTTGTCCCAACTGGCTGNCACTCCAAACCCGTCCCCCTGGCC
CCTGACCTTGCTGGGTGGGGAAAAGGCTACGTNGGGCCCAGGCTGAACAATGAATGANCATGTCAAG
CTGTCAAAATNTGGGAAATAAGNGCATTCTACTGGCAACCCGCCTTCCTGATCCCGNCTGGATTTGN
ACCATAAAGNGACATGAGAAGCCCCCCTGTGTNTGTTTTTGTGTGCCCCCCCCCCCCCGGGGGNNTNC
CNTNGNNAGGGGNTTTGGNANNTTTTTTGGTTTTGGAAAATTTGAAAGGGCCCAAAAAAAAAACNGG
GGGGTTTCCTTTTGAAAAAAAAAAAAAAAAAAAAAANTTNGGGGGGGGGGGCCCCN
TNNACAAAACTGGACTCCCCGCGGTGGCGGCCGCTCTAGAACTAGTGGATCCCCCGGGCTGCAGGGT
TTCCAGGACAGCTGAAGGGAGAGGAAGCTGGGTGTCTCCCAAGAGTCCCAGGCTGCAAAGGGGAGAG
AGCTGACCCTTCTCAAACCCCTGCTTTGGGCCCAGCCCTGCGCCAGGTGCTTCACAGCTCTTATCTGCC
AGATTTTCCCACCTTGTGGCACATGGCGCCCCTAACCAACATCCCAAGAGGCCGATCACCTTGCCACA
TCTGTGCAAGACCTGGGACCACAGGCGTGGACAGGAAGGGCTCCGAAGCCCTCTTTTTACAGATGAAG
ATGCCAGGGCCCAGGGAGGATGAGCAGTCTTTGGGGTTACCCAGCCGGAATACTGGGAGACCTGAAC
CCAGCATTCTGACTCTGCATTCAGTGCTCGCTCCATGATGCAGCGGTCCTAGCCCCCCCCTCAAATGGG
TCCCCTCTTCACTGTGTTTCCCAGCATCCTTGCTCTGACCAAACCGGGGAAGGGACACAGCCCACCCCC
AAAAGATGGAATCATTCTCTTAACCGTCTTTGTCCCAACTGCCTGCCACTCCAAACCCGTCCCCCTGGC
CCCTGACCCTGCTGGGTGGGGAAGANGCTTCACGTGGGCCCANGCTTGAGCAGTGAATGAACATTGTC
AAGCTTGTCAAACTTTTGGGAAAATAANGCATTCTAACTGGGAANCCCGCTTNCCTGATCCGNCTGGA
TTTTGGNCNTTAAAGGGGACTTGGAAAGCCCCCTGNGGGGGGNANATGGGGGGCCCCCCCCCCCCCG
NGGGGAAAACCCCTAAGGANANGGGGTTTTGGGACACCTTTTGGGGTTTGGGGAAAATTTGANAGNG
CCCAAAAAAAAACCNGGNGGGTTTTTTTTTTTNAAAAAA
TTNNNCAAANCTGGAGCTCCCCGCGGTGGCGGCCGCTCTAGAACTAGTGGATCCCCCGGGCTGCAGGG
GCGTCAAGGTTCTCCGTGGAGTTGGAGCTTTGGCCTCCCAGGCCCTGAGGGCCCGGGGTCCAAATGGA
GTCTCCGTGGTGCGCTCTATGGCGTCTGGAGGTGGTGTTCCTACTGATGAAGAGCAGGCGACTGGGCT
AGAGAGGGAGGTCATGCTGGCTGCTCGCAAGGGACAGGACCCATACAATATACTTGCCCCAAAGGCA
ACCTCAGGTACCAAGGAGGACCCTAATTTAGTCCCCTCCATCACCAACAAGCGGATAGTGGGCTGCAT
CTGTGAAGAAGACAACAGTACTGTCATCTGGTTCTGGCTGCACAAAGGCGAGGCCCAGCGATGCCCCA
GCTGTGGAACCCATTACAAGCTGGTGCCACACCAGCTGGCCCACTGAGCCACTGCACTAATGCACTCA
GAATATGATGTGGAATTTCTTCTTTCCAGTAAAGACTAGCCATTACATTGGCTTCTCCTCCCATAANAA
AAAAAAAAAAAAAAAAANCCCNNGGGGGGGGNCCNGGCCCAANTTCCCCNTATNGGGNGNNNNNNA
NANNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNC
TTNAGCCTATAGTGAGTCGTATTACAGGGGCGGCCGCTCTAGAACTAGTGGATCCCCCGGGCTGCAGG
GGCGTCAAGGTTACTGCGTGGAGTTGGAGCTTTGGCCTCCCAGGCCCTGAGGGCCCGGGGTCCAAATG
GAGTCTCCGTGGTGCGCTCTATGGCGTCTGGAGGTGGTGTTCCTACTGATGAAGAGCAGGCGACTGGG
CTAGAGAGGGAGGTCATGCTGGCTGCTCGCAAGGGACAGGACCCATACAATATACTTGCCCCAAAGG
CAACCTCAGGTACCAAGGAGGACCCTAATTTAGTCCCCTCCATCACCAACAAGCGGATAGTGGGCTGC
ATCTGTGAAGAAGACAACAGTACTGTCATCTGGTTCTGGCTGCACAAAGGCGAGGCCCAGCCGATGCC
CCAGCTGTGGAACCCATTACAAGCTGGTGCCACACCAGCTGGCCCACTGAGCCACTGCACTAATGCAC
TCAGAATATGATGTGGAATTTCTTCTTTCCAGTAAAGACTAGCGATTACATTGGCTTCTCCTCCCATTN
NANANAANNNNNNNNCNNAAAACNTGNGGGGGGGGCCCGNNCCCAATTTCCCCTTNAAGGGGNGNC
NNTTACANNNNNNNNNCCNNNNNTNNNNNNNNCNTNNNCNANNNNNNNTNTNNNCNNNNNNNNNNT
NNNNNNNNNNNNNNNNNNNNNNNNNNNNNNNNNNNNNTCNNNNNNNNCTNTNTTNNNNTNNNNNN
NTNNNNNNNNNTNNNNNNNNNNNNNTCNNNNTNNNNNNNNCNNNNNNCNNNNNNNNNNNNNNNNC
NNNANNNNNNNANNNNNNNTNNNNNNNNNNNNNNCNNNNNNNNNCNNNNNNNNTNNNNNNTNNNT
NNTNNNNNNNNNNNNNNNNNCNNNNNTCTNNCCNNNNTNCNNNNNTNNCNNNTNNNTCTNNNNNN
NNNNNNTNCNNTCNNNNNNNCNNNNNNNNNNNTNNCNNNNNNNNNNNNACN
NNNNANCTGGAGCTCCACCGCGGTGGCGGCCGCTCTAGAACTAGTGGATCCCCCGGGCTGCAGGTTTT
TCTCAAAGAGGGCAGCAGACTGTTCTCCCCACAGGCCATGGGGGGACAGGGCTGGGAGAAGAAATAG
ACGTGCACCTTATATCATGTAAATATACTGATTTTTCTAGTTCAAGAAAGAATTATTGGTAGAGTGGGA
ACGGGAGATAAGAATGAGCAGGAAATCTTGAGTAAGCCAGCTGGCTTTTAAGCCAAAAGGGTTCCTC
CGTGTTTATCTTTGAGACAGTCCAACCCCAAGAATAGCTTTAAAAGGGAAATCAGTGCTGAGACGGTG
AAGTCCCTTACCCAACACGCGCCCTGTAACTTTAGAATGAACGCAGGCTCCTCAGCTGTGGGCTNGGC
ATGGNTCCCGTCACCCGTTGGTGTGACAAGGCGGGAAATTCATTAGGATGNTTATGGNTTCTTNAT
CCCACCCAGCTTTTCCTGGTTCTGGATTCTAAACAACCCANAACCATCTCTTTCCTCCGGTGGCTCTGCT
TCTAGACTTGCGTGGGCCCTCAGACCCGTTTCTTGGCTTGGGTTTCCGGAAGCCTTCGGCAGACTTATA
CACGCATGCTCTTGCTGTCACATTTATTNCTTTGCCCTGCAGCCTTCTTTTGGGGGGGAAAAAAAAGTG
CNCTTACTGGACTGTNGCCNTTTTACNGTAGCCCAGTGTNTTTTTCACAGANTGGCCCTATCCCTTTTTT
TTTTTTTTTAAAACAGNGCCCCCCTCCCCTCTTNTTTTNTTTTTTNCNTNCNANGANCTTTTTTTTTTNAN
NGGGCNACCCCCCNTNTTGNAAAACCCCCCCCGGGGTTNNGGGGGCCCCTTGGGAATTTTNTTCCCNA
TTTNGGCAAATCCCCCCNTGGNNCCNNNGCTCCCTAAAAATTTTNTTTTTTTTNNNNTC
TTNAGCCTATAGNGAGTCGTATTACAGTGGCGGCCGCTCTAGAACTAGTGGGATCCCCCGGGCTNGNN
GNATTTCGGCACGAGGCCCGTCTCCTGTGTTAGGTCTGACCTCCGGCCGATCGTCCGCCGCCGCACGG
NTTGCCGCCATGGCCCTNANCGATGCCGACGTGCAGAAGCAGATTAAGCACATGATGGCTTTTATTGA
ACAAGAAGCCAATGAGAAAGCAGAAGAAATTGATGCNAAGGCAGAAGAAGAGTTCAACATTGAGAA
AGGTCGTCTTGTCCAAACCCAAAGACTGAAGATTATGGAATATTATGAGAAGAAAGAAAAGCAGATT
GAGCAGCAGAAGAAAATTCANATGTCCAATTTGATGAATCAAGCGAGGCTCAAAGTCCTCAGAGCGA
GAGATGACCTTATCACAGACCTACTAAATGAAGCAAAACAGAGACTCAGCAAAGTGGNAAAAGATAC
AACCAGGTACCAAGTGCTGCTGGATGGACTGGTCCTCCAGGGTTTTGTACCAGTTGCTGGAGCGCCCG
GATGATCGTTCGCTGCAGGAAAACAGGATTTTCCTCTGGTGAAGGCTGCAGTGCAAAAAAGCAATCCC
TGTGTCCAAGTTGCAACCCAAAAAGANACGTTTGATGTCCANATTGACCANGAAGGCCTTACCTTGNC
CCGAAGGAAGATANACCGGCCGGTGGTCCGAGAATCTATTAACCGGGGGACCCGCAAAGAATCAAAG
GGTGGTCCCACCNCACCTCGAGGTCGGGTTGGACCTTCATNAGCCCCAACCAAATGAATGCCCCGA
ANGTGGCGGGGGAGGCCTTTGTTTTGGTGGCAAATGCCCACCAGGGAAGGTTTTTTGGACTTAACCCA
CCAGGAAGGTGCCTTGACCAAGGGACCCCTCCCCTTNNTTTGGGAANAAAACNAAAATGGGCCCNGT
NTTAACCTTTCTTTTTTTCTTGTCCCCTTGCCCTTTTTTTTGGGNC
TTNAGCCTATAGNGAGTCGTATTACAGTGGCGGCCGCTCTAGAACTAGTGGGATCCCCCGGGCTNGNN
GNATTTCGGCACGAGGCCCGTCTCCTGTGTTAGGTCTGACCTCCGGCCGATCGTCCGCCGCCGCACGG
NTTGCCGCCATGGCCCTNANCGATGCCGACGTGCAGAAGCAGATTAAGCACATGATGGCTTTTATTGA
ACAAGAAGCCAATGAGAAAGCAGAAGAAATTGATGCNAAGGCAGAAGAAGAGTTCAACATTGAGAA
AGGTCGTCTTGTCCAAACCCAAAGACTGAAGATTATGGAATATTATGAGAAGAAAGAAAAGCAGATT
GAGCAGCAGAAGAAAATTCANATGTCCAATTTGATGAATCAAGCGAGGCTCAAAGTCCTCAGAGCGA
GAGATGACCTTATCACAGACCTACTAAATGAAGCAAAACAGAGACTCAGCAAAGTGGNAAAAGATAC
AACCAGGTACCAAGTGCTGCTGGATGGACTGGTCCTCCAGGGTTTTGTACCAGTTGCTGGAGCCCCCG
GATGATCGTTCGCTGCAGGAAAACAGGATTTTCCTCTGGTGAAGGCTGCAGTGCAAAAAAGCAATCCC
TGTGTCCAAGTTGCAACCCAAAAAGANACGTTTTGATGTCCANATTGACCANGAAGGCCTTACCTTGNC
CCGAAGGAAGATANACCGGCCGGTGGTCCGAGAATCTATTAACCGGGGGACCCGCAAAGAATCAAAG
GGTGGTCCCACCNCACCTCGAAAGGTCGGGTTGGACCTTCATNAGCCCCAACCAAATGAATGCCCCGA
ANGTGGCGGGGGAGGCCTTTGTTTTGGTGGCAAATGCCCACCAGGGAAGGTTTTTTGGACTTAACCCA
CCAGGAAGGTGCCTTGACCAAGGGACCCCTCCCCTTNNTTTGGGAANAAAACNAAAATGGGCCCNGT
NTTAACGTTTCTTTTTTTCTTTGTCCCCTTGCCCTTTTTTTTGGGNC
TNNNNAAAAGCTGGAGCTCCACCGCGGTGGCGGCCGCTCTAGAACTAGTGGATCCCCCGGGCTGCAG
GAATTCGGCACGAGGGCCGAGAAGGCAACATGGGTCACCAGCAGCTCTACTGGAGCCATCCGAGAAA
ATTCGGCCAGGGTTCTCGCTCTTGCCGGGTCTGCTCCAACCGGCAGGGTCTGATCCGGAAATACGGCCT
CAATATGTGCCGCCAGTGTTTCCGCCAGTATGCGAAGGACATCGGCTTCNTTAAGTTGGACTAACCAA
ACTTCCTTANATGGNTCATCCAGCACATCANCCTTAGGCGGAAACAATNNTAGNTNTTTGTNTATAAA
ATAAANGTTTTCAAANCTTCAAAAAAAAAAAAAAAAAAAAAAAANCCNNNGGGGGGGGGNCCGGGC
CCCNANTTCCCNTNNNNGGGGGGNNTNAAAAAANNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNCNNNTNNNTNNNTTNNNTNNN
NGNNNNCNCNNNNNNNNANNNNNNNNNGNNNNNCCCCCCCCCCCCCCCCCCCCNCCNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNGGGGNNNGNNNNTTTNNNNNNNNNNNANNNNN
NNNNNNNNN
TTNANCAAANCTGGAGCTCCCCGCGGTGGCGGCCGCTCTAGAACTAGTGGATCCCCCGGGCTGCAGGA
ATTCGGCACGAGGCCCANTCTCATCCCGTCCTCTCCCCTGGTTTGTAAATTGCTGCACTGGTAACATCAG
GTAACCTAGGAAAGAATCTGCCTGCAATGAAAGAGACTGGGGTTCGATCCCTGGGTGGGGAAGATCC
CCTGGAGAAGGAAATGCANCCCATTTCCAGTNTTCTTGGAGAAGGAAATGGCANCCCACTCCAGTANTN
TTGCCTGGANAATCCCNTGGACAGAGGANACTGGCCGGTTGGAATAGGACTGACCACCTACCATTTTC
CCACCNTTTCCACTTTGGAACTTTCCAGTTTTNGGAAGCAAAAAAATGGACTTCAGGCTTGGTTNGGAA
ATAACCTACTTTTTTTGNGGGAATNACCTTTTCATTTGNNAAAATAAAATANGGTTTTCTCNTTTCTGN
TTCNAAACCNNTAAAAGGGTGGAAGNAAAAATTTTCGACCTATGGTTATTAAACTNGATTTGNAATAT
NCCTTGAAAGCTTACCTTGGAAACNTACCTTATANTTTNCNTGCCCCAAATTTTGTTTGTTTTAAAATT
ANAANGGAAAAAATGGATNTAAGTTAATTTAATAAATTAATTGGNCTGGCCTTGGGATTNAATCCTCN
CAGCAATCCTGGTTGGGNCTGGTTTNCCCATTTTGGATNGGGNAATGGNGGNCCCGGGNAATTTTTTN
CTTTTAACCCCTTGAATGCCANGTTAAAAAGGGCTTTCCTNGGGGGGGNCCTTNNGGGGNAAAAAAAA
CCCCCCCCTTGGCCANNANAACCTTAAAAAAAAAANGGGGTTTGAANCCCCTGGGGCCCCCAAAAA
ACCCTTTGGGNAAAAGGNAAANGGCAATANCCCCTCCCAAAANATTCNCNCCCNGGGNAAANGNCCC
TTGGNAAAAG
GANACATTTGGAGCTCCACCGCGGTGGCGGCCGCTCTAGAACTAGTGGATCCCCCGGGCTGCAGGGTC
GGTCTGGGTGTCANNTGCTCCAAGGAAGTAGCCACTGCCATCCGTGGGGCCATCATCCTGGCTAAGTT
GTCCATCGTCCCTGTGCGAAGAGGCTACTGGGGGAACAAGATCGGGAAGCCCCACACGGTTCCTTGCA
AGGTGACTGGCCGCTGCGGCTCCGTGCTGGTGCGCCTCATCCCTGCCCCCAGAGGCACTGGCATCGTC
TCCGCCCCTGTGCCCAAGAAAGCTGCTGATGATGGGCCGGATTCGACGACTGCTACACTTTTGCCAGG
GGCTGCACTGCCACCCTGGGCAACTTTCGCCAAGGGCACTTTTGATGCCATTTTCAAGACCTATAGTTA
CCTTACCCCTGACCTCTGGAAAGAGACGGTGTTCACCAAGTCTTCATATCAAGAATTCACTGACCATCT
TGNGAAGAACCACACCAGAAGTCTTCGTGCAAAGGACACANGGCCCAGCTGTAGCCACCACATAATT
TTATAAAGGGAATAATTAAGGTGAATGAAACCAAAAAAAAAAAAAAAAAAAAAAAACTTGGGGGGG
GGCCCGGGCCCCNATTTCNCTTTATGGGGGGGGNNNNTNANAAANNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNN
TNNNNAAAAGCTGGAGCTCCCCGCGGTGGCGGCCGCTCTAGAACTAGTGGATCCCCCGGGCTGCAGG
GTCGGTCTGGGTGTCAAGTGCTCCAAGGAAGTAGCCACTGCCATCCGTGGGGCCATCATCCTGGCTAA
GTTGTCCATCGTCCCTGTGCGAAGAGGCTACTGGGGGACAAGATCGGCAAGCCCCACACGGTTCCTT
GCAAGGTGACTGGCCGCTGCGGCTCCGTGCTGGTGCGCCTCATCCCTGCCCCCAGAGGCACTGGCATC
GTCTCCGCCCCTGTGCCCAAGAAGCTGCTGATGATGGCCGGCATCGACGACTGCTACACTTCTGCCAG
GGGCTGCACTGCCACCCTGGGCAACTTCGCCAAGGCCACTTTTGATGCCATTTCCAAGACCTATAGTTA
CCTCACCCCTGACCTCTGGAAAGAGACGGTGTTCACCAAGTCTCCATATCAGGAATTCACTGACCATCT
TGTGAAGACCCACACCAGAGTCTCCGTGCAGAGGACACAGGCCCCAGCTGTAGCCACCACATAATTTT
ATAAAGGGAATAATAAAGTGAATGAAACCNNNNNNNANNNNNNNNNNNNAAAACTTGGGGGGGGGC
CCGGGNCCCANTTTCCCTTNTANGGNGGGNNNAANNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNN
TTNNNCANNNCTGGAGCTCCACCGCGGTGGGGGCCGCTCTAGAACTAGTGGATCCCCCGGGCTGCAGG
CTATACATCACTGTTCTTTTGCTGTCTCGTACGCAGCATAGCCTCTTAAAATTGTATCCTGACAAAGAC
CACATCAGCACATTTACCCCGCACTGATCCACTAGATGGCGCTGTCTCAAGCTGGCAGAACACACCAGG
TAATTTTTTGACAAGTTCTTTCTGNGTCTCATAAAATAGAGGTAAAGCTAACATTCTTTACTGATGCGC
AGTATCAGATATTTAACCTAAAAATTCACCAGAGTTATTGTGCGCTGTTGCGTTGCTAGGAAATGTACT
ACCATTTCACCCACATTGCACACAGCAGGTGTCTCGGAGCTCATTGTGTGCTCACAGAACGTGAAGCGT
GGTCCTTGTGGAGTAAATCCACTCGAGAACGAGGCTCTGAGGCTTCTCGGCTGCCTTCCTGACTCCGTC
CTGGGATGAAGACATCCAAATCATTTCTAAATGTTTTTACTCTCACATAAAGTAAGCCATTTTCTTGTTC
TGTTCTCTGAGACAGACTTTTCTATCAACTCTGTCCTGTCGTTGCTGCAGTTGAAGGATTTTGTTTTGCT
TTGATTTGCATTCGAGGGTCCAGCAACGTGCTGTGGGGACGTCCCTGGTGGCCCAGTGGTTACAGAAT
CCGACTGCCAACGCAGGGGACACAGGTTCAATCCCTGATCCAGGACGATCCCACATGCCGCGGAGCA
GCCGAGCCCACGTGCCACTCAGTTGGCCCGTGTGCCACTGGTTGACGCCATGNAAACGAGAAGTCCTG
GTACCCCAACNAAGAAGTANCCCCCACTTANNAACCAAAGACTTGGNCCAACCCAAAANTNANTTAA
AANTCCCNAAAAAAAAAAAAAANCCTNNGGGGGGGGGGCCCGGGCCCCNANTNCCCCCTNNNNNNGN
NNNNNNNNNNNNNNNNAAANNNNNNNNNNNNNNC
GCCTATNNGGAGCTNCANCNCNGTGGCGGCCGCTCTAGAACTAGTGGATCCCCCGGGCTGCAGGATCA
TATAGTAAACCCAAGCCCTTGACCTCTTACAGGAGCTTTGTCTGCCCTCTTAATAAGATCCGGCCTAAC
CATGTGATTTCACTTTAACTGAATGACCCTGCTAATAATTGGCCTAACAACAAATATACTAACAATATA
CCAATGATGACGAGATGTTATCCGAGAAAGCACCTTCCAAGGGCACCATACCCCAGCTGTCCAAAAAG
GCCTCCGTTATGGAATAATTCTTTTTATTATCTCCGAAGTACTATTCTTTACCGGATTTTTCTGAGCTTT
CTACCACTCAAGCCTCGCCCCCACCCCTGAACTAGGCGGCTGCTGACCCCCAACAGGCATTCACCCAC
TAAACCCCCTAGAAGTCCCACTGCTCAACACCTCTGTCCTATTGGCTTCCGGAGTTTCTATTTACCTGAG
CCCATCATAGTTTAATAGAAGGGGACCGAAAGCATATATTACAAGCCCTATTTTATCACCATCAGATTA
GGAGTCTACTTCACACTACTACAAGCCTCAGAATACTATGAAGCACCTTTTACTATCTCCGACGGAGTT
TACGGCTCAACTTTTTTTGTAGCCACAGGCTTCCACGGCCTTCACGTCATCATTGGGTCCACCTTCTTAA
TTGNCTGCTTCTTCCGCCAATTAAAANTTCATTTTACTTNTAACCACCNCTTCGGCTTTTGAAGCCCGCT
GGCTGATACTGACATTTTNGNAAACGNANNCNGGACNTTTCCCTTATGGNTCTATCNATGGGGGAGNC
TCCTNNNNNNNNNNNNNNNNNNNNNNNNNNNNNNNNNNNNNNNNNNNNNNNNNNNNNNNNNNAAA
ANGGGGGGGGGGGGGGGNNNNNNNNNNNNNTTTTTTTTNNNNNNNNNNNNNNNNNNNNNAAAANNN
NNNNNNNNNNNNNNNNNNNC
NAAAAGCTGGAGCTCCACCGCGGTGGCGGCCGCTCTAGAACTAGTGGATCCCCCGGGCTGCAGGAATT
CGGCACGAGGGAGAGAGAGAGAACTAGTCTGAGTTTTTTTTTTTTTTTTGGNGAANTTTTTTTNNGNA
CCNGCCACCNNGTTNTNGTNAAAGGGGGNANTNGAACCTNNGGGGGNCCNNCGGCCNGCNANNANTT
NGCCCTAAAANGNGCCCCTTTNGNAAANNGGGGGTTTNNNCCNGGNCCNCGNCCCCTTTTTTTTNGNC
CGGGGGGCCCNNNCCNAAAANNTTTTTTTTTNNTNGTAANCTNGNTTTTAAANNTTNNTTTNNCGCCNT
NAAAAAAANTNNGTTTNGGGCNNANTTGNCCNAAAACCCTTTNCCANGNNGNNTTTTNGTTTCCCAAA
AAAAAANTNCCNTTTCCCCCAAAAAAAAAAAAAAAAACCNNGGGGGGGGNCCCGGCCCCCNATTCCC
CNTNNNGGGGGGNNTNNNANANNNNNNNNNNNNNNNNNNNNNNNNCNNNNNNNCNNCCNNNNNCN
NNCNNNCNNNNNNNCNNCNNNNNNNNCCNCNNNNNCCNCNNCNNNNNCNNNNNNNNNNNNNNNNN
NNNNCNNNCNNNTNNNCNNNNNNNCCNCNNCNNNNNNNNCNNNNNNNNNCNNNANNCNNCNCNCN
NNNNCNCNCNNNNCNNNCGNNCNNNNNNNNNCNNNNNNNNNCCNNNNNNNNCNNNNNNCGNCNCN
NNNNNNNNNNNNNNNNNCCCNNNNNCNNNNNNCNNNNCNNNNNNNCCNNNNCNNNNNCNCCNNCN
NNNNNNNNNNNNNNNNCTNCNNNNNNNNNCNNCNNNCNNNNNNNCNNCNNNNNNNNCNNNNNCCN
NNNNNNNNNCNCNNCNNNNCNNCTNNNNNNNNNNNTNCNNNNNNNNCNNNNCNNNCCNNNC
TTAAGNCCNTNNNGTGNNGTNNNNNTNAAANNGGANNACCGGTCTANAACTAGTGGGATCCCCCGGG
CTGCAGGNCANTGCATATNCNTGTTTGGNGTGTGGCAAGTACTTTCANGGCCGGGGNTTGAANTCTCA
TGCCTACATTCACNGTGTCCAGTTTAGTCACCATGTNTTCCTCAACCTCCACANCCTCANGTTTTACTG
CCTCCCTGACAACTATNAGATCATTGATTCCTNANTAGANGACATTACGTATGTGTTGAANCCCNCTTT
CACAAAGCAGCAGATTTGCTAACTTGGACAAGCNCGCCNAGTTATNCCGGGCATATGATGGCACCACTT
ACGTGCCAGGTATCGTNGGGGNGAATAACATNNANGGGAATTTGACNAGTCCAACTGCGGNCCTTTCAN
GCTCTGTCTAACCNTTNCTCCTNTCTGGANCTACCTTCTGGCAAGANGACNACTNTATANGAACATCN
NATNTCTNCANGGGGATATTATCGATTTTNNTNGGTCCCANNNGTTNNCGNANNAGCTNCNGGAGAA
AACTTTCTNNTAACCNCTTGCCAATTNCNAAAGCACNTGTCCTCCTTCCCCATTGAAACCCTCCACNCC
TNTCNGTCCTCTNGNCCACCCNTCNCNNCTTTTTANCAATNNACNNATACAGGGGGGGATCNGAAGGN
GNATTTTTACTNNCCCTGGGGCTTTTNGAANGGNTNANNCANTNGACCAATTGNNNGGGCCTCANNNG
AAGAAANANCNANNANCTNTTNGGTNCCTGANNGGTTANTNCCNGAGGGNNNCNNNGNAGGCTNNN
TTNNTTNANAAAANTNNGCCTANTTCTGGNTTTNTTNNTCGNNANACATTAAAAGAACATCCCTNNNG
NTCTNNANTGNANCANTTCCNCNCGGATNNCANNNCNNCGNTTTNNCCCTAAATATTNATTGTCTAGAG
CNANNTNGGANCCCTTTCNTACCCTGTNCTCCNAGNTCCTATGGGNATNCTAAATGTGGNANANGTTN
NNTANTATCCNNCCCGNCC
TTAAGNCCNTNNNGTGNNGTNNGTNNNNNTNAAANNGGANNACCGCTCTANAACTAGTGGGATCCCCCGGG
CTGCAGGNCANTGCATATNCNTGTTTGGNGTGTGGCAAGTACTTTCANGGCCGGGGNTTGAANTCTCA
TGCCTACATTCACNGTGTCCAGTTTAGTCACCATGTNTTCCTCAACCTCCACANGCTCANGTTTACTG
CCTCCCTGACAACTATNAGATCATTTGATTCCTNANTAGANGACATTACGTATGTGTTGAANCCCNCTTT
CACAAAGCAGCAGATTGCTAACTTGGACAAGCNCGCCNAGTTATNCCGGGCATATGATGGCACCACTT
ACCTGGCAGGTATCGTNGGCCNGAATAACATNNANGCCAATTGACNACTCCAACTGCCGNCCTTTTCAN
GCTCTGTCTAACCNTTNCTCCTNTCTGGANCTACCTTCTGGCAAGANGACNACTNTATANGAACATCN
NATNTCTNCANGGGGATATTTATGGATTTTNNTNGGTCCCANNNGTTNNCGNANNAGCTNCNGGAGAA
AACTTTCTNNTAACCNCTTGCCAATTNCNAAAGCACNTGTCCTCCTTCCCCATTGAAACCCTCCACNCC
TNTCNGTCGTCTNGNCCACCCNTCNCNNCTTTTTANCAATNNACNNATACAGGGGGGGATCNGAAGGN
GNATTTTTACTNNCCCTGGGGCTTTTNGAANGGNTNANNCANTNCACCAATTGNNNGGGCCTCANNNG
AAGAAANANCNANNANCTNTTNGGTNCCTGANNGGTTANTNCCNGAGGGNNNCNNNGNAGGGTNNN
NNTTNANAAAANTNNGCCTANTTCTGGNTTTNTTNNTCGNNANACATTTAAAAGAACATCCCTNNNG
NTCTNNANTGNANCANTTCCNCNCGGATNNCANNNCNNCGNTTNNCCCTAAATATTNATTGTCTAGAG
CNANNTNGGANCCCTTTCNTACCCTGTNCTCCNAGNTCCTATGGGNATNCTAAATGTGCNANANGTTN
NNTANTATCGNNCCCGNCC
TNNNNAAAAGCTGGAGCTCCACCGCGGTGGCGGCCGCTCTAGAACTAGTGGATCCCCCGGGCTGCAG
GCCGGTTTCCTGAGNCCCGCAAGGCGGCGGAGACATCTCTTTTGAGTAAACGACTCAGGGTCCAGTGC
TCCAGTGGAAAGATCTAAGGGGCCCGCCACTAGCGCCGCCACCATGCCCAACTTCTCTGGTAACTGGA
AAATCATCCGATCGGAGAACTTCGAGGATTTGCTNAAAGTGCTGGGGGTGAATGTGATGCTGAGGAA
NATNGCATTGGCTGCANCATCCAANCCANCAGTGNANATCAAACAGGAGGGGGACACTTTCTACATC
AAAACCTCCACCACCGTGCGTACCACAGAGATCAACTTCAAGATCGGAGAGGAGTTTGAAGAGCAGA
CTGTGGATGGGAGACCCTGTAAGAGCCTGGTGAAATGGGAGAGCGAGAACAAAATGGTCTGTGAGCA
NAGGCTGTTGAAGGGAGAGGGTCCCAAGACCTCCTGGACCAGAGAACTGACCAACGATGGAGAGCTG
ATCCTGACCATGACGGCGGATGACATCGTGTGCCCCCAGGGTCTACATCCGGGAGTAAGCGGCTGCGG
CCCCAGAGCCACCAAAGCTTGCCCCCCGNCCATGCTTCCTTGCTTCCTGNCCCACCCCTTTCTTTTAGG
CTAGCTTTTNCCCTTATTCCCAATCACTTTNTGGGGGGTTCTGGGGTTGCCTTTGCANGGGCTTTTCN
TTTTATTGGACCCCTTCNTTTTCNTCCCCATATTTTAAACCCGATGGGATGGGAGCCCCAAAATCACC
CGATTCCANGGATTAATTTCCCCCGAACCCCCCNCCCTGGGGCCTTANCCCCCAAAANAANCCCCGNA
NNAACCNTTTTTTTTTTNTTTTTTTTGNAAGGGGGANCCCCAGGGGCCTTAAATNNGGAAAAAAACGG
GCCCCTTTGGGCTTTCCANTTTTTTTG
TTNANCAAAGCTGGAGCTCCACCGCGGTGGCGGCCGCTCTAGAACTAGTGGATCCCCCGGGCTGCAGG
CCGCCGGCCTTGGGGCACTGTGCTGGTCGGCGGAGTAGGCGGCCATGGGGAGCCTCCGCGGCCTGCGC
CTGGTGGCTGGAAGTTGTTTTAGGTCATGTGAAAGAGACGCTTTCTCATCTTTAAGGCTTACCAGAANC
TCTGATTTGAAGAGAACAAATGGATTTTGCAGCAAACCCCAGGAAAGTCCAAAGCCTCCAGACCAAC
ACACTTACAGCCACAGAGTGCCATTGCATAAGCCTACGGATTGGGAGAAGAAGATTCTAATATGGTCA
GGTCGCTTCAAAAAGGAAGATGAAATTCCAGAGACTGTCTCGTTTGANATGCTCGATGCTGCAAAGAA
CAAGGTCCGGGTGAAGATCAGCTNTGTAATGATTGCCTTTGCCAGGGGCAGGATGCGTATTGATGGTT
ATAGAGGGCAAGAAGGCTGCCANAANAAATGANACTTTACCAAGCTTGAACCTAAAAAAAAAGCTTG
TNTGAAAAAAAAANCTTGTTTTGAAGGCCAAACCGAGTACCANATNTNTCGTNGGTNGGATTTTGAAN
ACCTAAAAATAATGCTGCAGCAATAGGCCTTATTAAAAANAANGGGGGGGTTGANCTTNCCTTNTTNC
CGTTTTAAACTTCNAATAAATTTTNTTAAANAAAAAAAAAAAAAAAAAACCTTGNGGGGGGGGCCCG
GGNCCCCAATTCCCCCTTTANGGGNGGNNNNNNAAAANNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNN
NNNNNNNN
TTNANAAAATCTGGAGCTCCACCGCGGTGGCGGCCGCTCTAGAACTAGTGGATCCCCCGGGCTGCAGG
AATTCGGCACGAGGGCGCTTCTTTGCTCGCTCCTCCGCACCCCGCGGCCCGTCTCCTCCATCGATCGCC
ATGGAAGAAGAAATTGCCGCCCTCGTCATCGACAATGGTTCTGGCATGTGCAAAGCTGGCTTTGCTGG
GGATGACGCCCCCCGGGCCGTGTTCCCGTCCATCGTTGGGCGTCCCCGACACCAGGGGGTCATGGTGG
GCATGGGGCAGAAGGACAGCTACGTGGGCGACGAGGCCCANAGCAAGCGTGGCATCCTGACCCTTAA
GTACCCCATCGAGCACGGNATTCGTCACCAACTGGGACGACATGGAGAAGATCTGGCACCACACCTTT
TACAACGAGCTGCGTGTGGCCCCCGAAGAACACCCCGTGCTGCTGAGNGANGCCCCTTCTAACCCCAA
GGCCAACCGTGAAAAGATGANCCAGATCATTGTTCGAGAACGTTTCAACACTCCTGGCATGTATGTGGG
CATTCCAAGAATGGGNAATANTTAGNCGACTGATGCCCGAACAATTGGGAATTGTCNTTGGACTTNTG
GGGGACGGGGNNCACCCCACACCGGTGCCCCATTTTACGAGGGGGTACGCCCTNNANNNNNNNNNNA
TNCTTGCGTTTTGGACCTTGGCTTGGCCGGGAACCTGGACAGAATTACCTTAATGAAAAATCCTTAACN
GGAACGNGGGTTACAGGGTTTTAACCACCCACAAGCCCGANCCGGGGAAAATTGGGGCCGNGGACAT
CAANGGANAAAACTTTTTGGTTACCTTNGCCCCTTGGAACTTTCGAAACAAGGAAAAAGGGTTAACCG
GGTGNAATCCCCCCTTCCTTCCTTTGGANAAAAAAACTNTTAANCTTGNCCCCCAANGGTCAAGGNGA
AAAACCCNTTGGGCAATTGAACCNGGGGTTCCGGNGGNCCCCANGGGACCTTTTTTC
TNNNNCAAAGCTGGAGCTCCCCGCGGTGGCGGCCGCTCTAGAACTAGTGGATCCCCCGGGCTGCAGG
AATTCGGCACGAGGCTTTTGTATTATCAAGTAAGATTCTTTTTTCGGCTGTATATAAGCAAGATTGTTT
GGTTTATTCTTTTCTTTTTTAGCGTAGGAAAAGTTTGTTGTTAAGGTATGCTTCAAAATTATTGTTAATT
GTATTTTGAATTTACATCTAAGCCTTGTAAATCATAACTGATAAGAGGGGGTGGGCTTTTGTTGATGAG
GGAGGGGAAACCTTTTTTTTTTGTAAACTTTTTTCAAATANCATNTTNTGAGTGATANCCAGCCTGGCA
ATCTGATGGCCTAAATGGANAGGGGANCAGCTCCTTGGNGAATGATAAGTAANGGCANAAAANATTG
TNTTTCATNCCTACNTTGGGGAAAAAAGCNTANCCCTGANATTTTNTTCTGATNAACACATTGNCTGCN
CATGCAAAAAAAAAAAAAAAAAAACNNGGGGGGGGGCCCGGCCCCAATTNCCCCTTANNNNNNNNN
NNTNAAANNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNN
ACAAANCTGGAGCTCCACCGCGGTGGCGGCCGCTCTAGAACTAGTGGATCCCCCGGGCTGCAGGAAA
TTCGGCACGAGGAGACATGGCCAAGTCCAAGAACCACACCACGCACAACCAGTCCCGAAAATGGCAT
AGAAACGGCATCAAGAAACCCCGATCACAACGATATGAATCTCTTAAGGGGGTAGACCCCAAGTTCCT
GAGGAACATGCGCTTTGCCAAGAAGCACAACAAGAAGGGCCTGAAGAAGATGCAGGCCAACAATGCC
AAGGCCATGAGTGCACGTGCTGAGGCTGTCAAGGCCCTCGTGAAGCCCAAGGAGATCAAGCCCAAGA
TGCCAACAGGTGGCAGCCGCAAGCTCAGCCGACTTGCCTACATCGCTCACCCCAAGCTGGGGAAGCGC
GCTCGTGCTCGCATCGCCAAGGGCCTCAGGCTCTGCCGGCCAAAGTCCCAGGCCAAGGCTTCAGCCAA
GGCCAAGCCACCTGCGGCTGCGGCTCCAGCTGCCAAGGGTGCCCAGGCCCCCACCAAAGCTCCGGAGT
AGAGACCTTCATCTGCCAGTGTGAGGACAGAAGGACTGGTGTGACCCCCTGGGCTGCTGTCTGCGTGG
GGCTGGTGTCCTCCTGTGCTATTTGTACAAATAAATCTGAGACAGGAAAAAAAAAAAAAAAAAAACT
NNGGGGGGGGCCCGGNNCGCATTTNNGCCTAAANGGGANGNNNNNNNNNAGNNNNNNNNNNNNNNN
NNNNNNNNNNNNNNNNNNNNNNNNNNNNNNNNNNNGNGNNNNNNNNNNNNNNNNNNNNNNNNNN
NNNNNNNNNNNNANNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NNCAAANCTGGAGCTCCACCGCGGTGGCGGCCGCTCTAGAAGTAGTGGATCCCCCGGGCTGCAGGAA
CGGCACGAGGCTTTGGGGTTCTTTCTCCGCGTAACGTGTGGTCTCTGATATTCAACCCTCCTCCGAG
CATAGGCTGCATGAGGCGGGGGCGGGCTCCCACATTCCTGCTCGGGGGAGGGGCCGCTCTGCTGCTGT
CGCTGTTTTGGATGCCGACACTGGTGCCTGCAGCCTCTCGCCTTCTGTTGCTACCCCGAGCCCTGCTGT
CCATGGCTTCAGGAAGTCCCCCGGCCCAGCCCTCGTCGGCGTCGGGCTCCGCGTATGTCCCCGGCTCGG
TCTCTGCGGCCTTTGTCACCTGCCCCAACGAGAAGGTCGCCAAGGAGATCGCCAGGGCTGTGGTGGAG
AAGCGCCTAGCAGCCTGCGTCAACCTTGTCCCTCAGATTACATCCATCTATGAGTGGAAAGGAAAGAT
TGAAGAGGACAGTGAAGTGCTGATGATGATCAAAACCCAAAGTTCCTTGGTTCCAGCTCTGACGGATT
TTGTTCGGTCTGTGCACCCTTATGAAGTGGCTGAGGTGATTGCATTGCCTGTGGAGCAAGGGAACTCCC
CGTACCTGCAGTGGGTGCGCCANGTTACANAATCNGGTNCTGACTCCAGTACAAGCCCCGCTGTGAAT
GAAGCTTTGGTCNTGGCCTGGGTTNCGTCATAAATATCAACACTTCTACCCTTGGAAGAACNGGACTCT
TAAAAAAATCCCGGTTTTAANTCTTTTCNNNNNNNNNNNNNNNNNNNNNNNNNNNNNNNNNNGGGG
GGGGGGGGCCCNNCCCCCNNNTNNNNNTTTNNNNNNNNNNNNNNNNNNNNNNNANANNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNN
TNNTNTAGTNANGNNTGCTNGANCNCACNGNNNCCTNTNAGNCCTATATGGAGCTGCATNACAGTG
GCGGCCGCTCTACAACTAGNGGATNCCCCCGGNCTGCNNGAATACGGGNTCCNAGNGNTTTTTTACTC
TTTTCCTANTNTNNCCGACNCNTTCANTNGGGGTANCTTGCAGNNNGATCTNAGGNNTCNCTTNAGAT
ATNNTNTGTTNNCTNCNAGTNGTCCTGTNGATCCCNTTACCNTNTGNNATNNTNTCATTCNNGNCAAN
TGNAAGNAAGGTANGTCNCCCTCAATGTANTATTNGTNTNCGNTNGCNGTCTTNNNNNNGNTTTCTA
TTCNNNNNCTTNNCNNNNNNTCCCTGNATTNATTNNTTNTATNTTCTCCCCNNTTGTNCANNANTNTC
NNNTNNNTANATCTGNNAACCNCCCNTTAGCTCNTANNNNNTANNTNNTNTGTGCNNCNTNCATTTTT
NNTANTNCNNCANNTANNNATCTACNGANNTAATNTTNTTAANGTNTTTNNCTCCNGTNTCCCCNNNT
CNNNTNTANCNNNTNTCNTTTCCACTTCGGGCTTNGNNNNANACNATNCNNNTNTTCNACNCCTNNTC
CNNCCCNNTTNNNNTTCCCGNCATATTNTCTTTAANGTANACTNTNCNATANCTGTTNTNTACTTNTTA
NGNTNNACCACATCTTNCTGTAAATNCCNACCTTTCNCGATNNANNCNNACANCGANTTANNNTTTTT
NTNTNNCTTTTTNCCTCTCTCCNNTNNTTNCNTTNNNTATCCTCNCTNNCNATNNNTCTNTCNNNNCCC
TTNTTNNTTTCCTTNTNATATCTACGNTATCCNNNTCNATGTCNNACTCTGTATTTTATNNCCTTAAGAC
NNTCANCANTNGNATNTTTTNTNNATTTNATTACTATCGCNNNNNNCCCNGNGCANCNTTNTNTTGGT
TCNNTCTCNCTNNTNTTNATCNANNANCTATCTCTGNAANNTCNGCTTCNNTNCATTNNAANTNTNG
CCANCNATTNNGTGTAATNTNTNTATTTNTCACNTNNTNTNAATNTTNTNCTCCTNTANGANNNTCACAT
ATCNNACNNTNNTGNTCNGCTCNTCACNTTNTATNTATCNTNTATATAGNTNNTATATNGATTGTTCTN
TNANGATATGCATANTNTNTGNTCATCTGTTATTGCACCTCGCNANACTNNTNNGTNANCTGNCTATNT
GCNCATCTATCTCCTTAAGNTCNACANCNCTTNNNGCGNCNCTGATNCTATNTATNTCTCNTCNTTANC
ATACATNCTNCTCCTNNTCNCCG
TTNAGCCTATAGNGAGTCGTATTACAGTGGCGGCCGCTCTAGAACTAGTGGATCCCCCNGGCTNGNNG
GNNANGGGCATATGCTTGGTCTGGGTGGTGTGGNAAGGACCTTTNAGGGCCGGGGTTGNAAGNTTAN
GNCTNNNTTTACNNGGGNCNANTTAANGNNCNTGGNTTTCTTAACCNTGANNCCGTTAAGNTTTANNG
GCNTCCTNNCNACNANNAAAACATTGNTTCCTNACTNAANGANNTTAGGTATGNGGTGAANCCCACTT
TNACAAAGCANCAGATATGCTAACTTGGACAAGCAAGCCAAGTTATCCCGGGCATATGATGGCAGCA
CTTACCTGCCANGTNTCTGTGGGCCTGAATAACATANAGGCCANTGANTACGCGAACGCCGNNCTTNN
GGCTCTGTNTAANGNNCCTCCTCTCNGGAACTACTTTCTGGNAGAAGACACTTATANGAACATCAAAC
GNCCTCCAGGGGATATCATGTGCTTGTTGGCCANCGTTTCGGAGAGCTGATGAGAAAACTCTGGAACG
CTCTAATTTCAAAGNACATGTTTTCCNCATGAAANGNTGCAGGCTGTTGTCCTCTGGNGCAAGAAGAC
TTTTTNTATCACGAAACAAGGGGATGGAGNTGATTTNTCTGNCCTGNTTNTTGAAAAGCTNTACAGTN
NANCTCTGGGGGGGNGCCCCNAAGNAAAAAAAAAAGACAAATTGTTACCTGGACGTTTTTCCANGGG
GGNGCCATTGAAGGATCTTTCNCTAAAAAAAANNGCCTTCATCCTTGATTTTGGCCANCAAAAAAAAA
AAAAAAANCAACNTCNTGCNTTATTTTACCGANTNNCCCCGGNNAACCNANGGGNNGGGGGGNCNCNA
NTTAATTTACCNTGGCGNCTTGGNANGTTTCCTTTNCCTCCCCCCCTGTTNCNNGGGACGGAAAAANG
GANACNGGNTTCATTTTTTCCCCNNNGGGGNTTN
TTNAGCCTATAGNGAGTCGTATTTACAGTGGCGGCCGCTCTAGAACTAGTGGATCGCCCNGGCTNGNNG
GNNTANGGGCATATGCTTGGTCTGGGTGGTGTGGNAAGGACCTTTNAGGGCCGGGGTTGNAAGNTTAN
GNCTNNNTTTACNNGGGNCNANTTAANCNNCNTGGNTTTCTTAACCNTCANNCGCTTAAGNTTTANNG
GCNTCCTNNCNACNANNAAAACATTGNTTCCTNACTNAANGANNTTACGTATGNGGTGAANCCCACTT
TNACAAAGCANCAGATATGCTAACTTGGACAAGCAAGCCAAGTTATCCCGGGCATATGATGGCACCA
CTTACCTGCGANGTNTCTGTGGGCCTGAATAACATANAGGCCANTGANTACGGCAACGCCGNNCTTNN
GGCTCTGTNTAANGNNCCTCCTCTCNGGAACTACTTTCTGGNAGAAGACAGTTATANGAACATCAAAC
GNCCTCCAGGGGATATCATGTGCTTGTTGGCCANCGTTTCGGAGAGCTGATGAGAAAACTCTGGAACC
CTCTAATTTCAAAGNACATGTTTTCCNCATGAAANGNTGCAGGCTGTTGTGCTGTGCNGCAAGAAGAC
TTTTTNTATCACGAAACAAGGGGATGGAGNTGATTTNTCTGNCCTGNTTNTTGAAAAGCTNTACACTN
NANCTCTGGGGGGGNGCCCCNAAGNAAAAAAAAAAGACAAATTGTTACCTGGACGTTTTTCCANGGG
GGNGCCATTGAAGGATCTTTCNCTAAAAAAAANNGCCTTCATCCTTGATTTTGGCCANCAAAAAAAAA
AAAAAAANCAACNTCNTGCNTTATTTACCGANTNNCCCCGGNNAACCNANGGGNNGGGGGGNCNCNA
NTTAATTTACCNTGGCCNCTTGGNANGTTTCCTTTNCCTCCCCCCCTGTTNCNNGGGACGGAAAAANG
GANACNGGNTTCATTTTTTCCCCNNNGGGGNTTN
TTNANAAAAGCTGGAGCTCCACCGCGGTGGCGGCCGCTCTAGAACTAGTGGATCCCCCGGGCTGCAGG
AATTCGGCACGAGGCAAAAAGCGATTGCAGTTGTTGATGGGGACCGAGATAAGGCCCAGAATTCTGA
CCAGATACTTGACACTTCATCACCCGTTCCTGGCTGTTCCTCTGTAGCTAACATCACATCATCTCAGAC
AACCTCACGACAACAGGGACTGGCACATCCAACTCGAGGAGGTGATGCTGAGGCTGAAGACACAGTG
AGCGAGCACCCAACTTCTAGCAGCGGAGCCCCAGCGCCTTCCTCATCTCAAGCTAGTGAGGGTCTCCC
TCAGCATTGTGCCTTAGGTCAAGTGGAGGATCATCCTTGGCAGCTCANAGAACCTTGTTANTTGATAG
AATACCAAAGGATACAATTGNCGGATCCGACTGAACACCACTTAAAAAGGAANCCGAATCCTGANAA
CTTGGCCCGNCTNTNGAGTGGGNCCGAAAAAAAANCTTANTACATNGNAANGCTTGGAATGGCNNCC
CAAAATTCCCTTGCCCCGGCCCATTAATTTTNAAANCCATGNGNTTACCTTTNCAGGNCCCCGGGAATC
ATTCGAGGGGCTTGNCAAAAGGAAAAGGGTCTTCCCATNTTCTTNAAAGGAAAAGGGGGCNCAAGGA
NCAGGAAACATTTTTGATGNATTTACAAGGGGNCCCGGNTTTTGGCCGGTTGGACCAACTTGCCAACC
CAAGTTGNTTTTCATTTANAAGGAATCGCTTGGGCCNTTTNAAAAAACCGCCNAAAANCCCAAGTTTTT
TANGGGGAATCCAAGNCCCAAGCTTTCCAAANAAAAAAAACTTTTCTTTNAAANAATTAAATNTTTAA
AAANGGCCGAACCTTTTAATNCCANAAAGGGGGGGTNNTTTCANGGGAAAAANCCCNAAAAAATTAA
GGGGATTNAAAAAANAAATTAATCCAANCCCCTNCCNNGGGATGNAAATTTTNG
TTGANAAAGCTGGACTCCCCGCGGTGGCGGCCGCTCTAGAACTAGTGGATCCCCCGGGCTGCAGGAAT
TCGGCACGAGGCTTNNNNACNCCNTCGAACTCNANGGGNNNCCNGGGNCCCAATTCGCCTATAGAGG
ANTCGNNTTACANTCCTGAAGGNNNCCNNAANANCCCTNTNNGAAATTNCATGCNCCNGNNATNNCN
CCNTCCTNANCTNCNGAATGAANAANNTGGNCTTGATCCTNNCNAAGNTGGCAATNCGNATNCTATN
AAGTCCTTTNNNACCGTGGAANTNGGCCAAACGTGCNTAAANNCCATTCTTTTGNTTATCCCNCAGGA
TTACTGGTNACCATTTCCTGNCGANGAGAACANNCNTNTTGGGTTTGNAGACTNCATGNAAGGNCNGN
TTTTTAGTTNANCTTTGCCTATCCTGAACTTGCCGAAGACGGACGTGGNTNCCCTGGTTGNCNTTCCNC
CTGGGTTTNCTNCAGCCNGGGCCTTNTCAAAAAAATTACATTTTCNCTGNGNAGNATTNCCATTGGCA
TACATTGGNTTATTGCAGNGGGGAATNTAAANAAAAGCCTTGAAACTTAATGGGGTCAAATGAAAAG
TGAAATTCNANGGNTTGAANGGAAAAAAACCAAATTTNCCATTCCCNAGTTTTGGNNGGAATGGGTT
GNACCAAAAAANACCCCTTGGGGGGAAATGGGGGCCAAAAACAGNNTTTNCNTTATTTAAAACCCCC
CAAGGGCCGGNAANACCTNCCTTANTTNGGANAATATTNGCCCCCCTTNTGGANNTTCGGGNGGGGN
CNTTGAATNAAANAAATTTGGGCNGAAANCTTTGGGCCCCCGGTTTGGCCTTTTTNATTTNAAACCCAA
ANTTNTTTTTTCCAANGTTCCCAAAAAAAAAACCTTTNCACCTCCCAAATTNNCCCTTTTGGGGNTTAA
AANTTTGGCGNGCCCCGTCNANANGGGCNCCANGTTCCCCCGTTTTTTTNTTTTCCCNAATTNTNTGNG
GGAAA
TNNANNANAAGGTGGAGCTCCACCGCGGTGGCGGCCGCTCTAGAACTAGTGGATCCCGCGGGCTGCA
GGNTTGTATGTACAGGGCANTGATGTAACAACAAAAAATACCTGAGAAACANAATGTCCATGCTTTCT
GCATCTTTAACACTTTGTGTTTGTATCACCTCATGGTGGCTTATTTTTCCTGTTGTACCATCCAGTCAGA
TTTTGAATAAACGAGTTTTGAAAAGACAGTACCTTTTACTATAATTGCCATTAGCAAAGTAGCCCTGGG
TACTTCATGAGAACCGGTATTAAAATATTAGTGGGACGGCTGTTTTCTGAAGCTTCGGAAGTANCCAC
ATCAGGGACCTAGGATATTTTTCTGAGGTCACTGGACAACTAGACTATGNATTGGCTGCTATNCCCTG
ACCTGTANTACAGAATGATGCCTTGGCTATGTAATGGAATATATTCTAAGGGGGATGGTGTATATTAA
GGGACTTTNATTCACAAGTACCATANTGGATATTCCAATGTATGGNAAAAACCATCTGGTGGGTTGGG
GNCANTTTTTAATTTACAGGTTTGNATTAGAACCATAANATGAAAACCTCTGGGGAAAAAANTTTTGA
ATGGGCAGCCCCACCCCCAAACTATTTTTTAAATGANTTCATTANAANATTTTCTTCAATAAGGGNCTT
TAATTTTTCCTTTTTGAAACCTATAAAATACCGTCAANAAAAAAAAAAATCAAGTTATCTTTNTTTCTAT
CAACTGGTTTTGGACCTAGTTTTAAATATAAAANGGNCCTTANATTTGNAANACCTTTTCTTTTGCCTT
ACACTGGCCTCANTTATAAAAACAGGGCANTTTCNCGCTGGTTCANGGGGATCACAAGGGACCTCAAC
TGGAAANTNAAAANTTTTGGTATCNAANAAATGTCCGNCATNGGCAATGGTNCCNCCCCCCCAATTAA
AAATACCTTTTCCAAGCTTGGGATNCCCCNAAAAAATNTTTTAAACCGGGG
TNNNACAAANCTGGAGCTCCCCGCGGTGGCGGCCGCTCTAGAACTAGTGGATCCCCCGGGCTGCAGG
NAAAATATGTGTTTCNTTTTGNCACNTANAANCTGANACTGANNNNNGTTGNNGGANANGANATNAC
AGNTGANTTCTGNNTGGAGTTGATGAGCCCAAGGACTGGGCTGTTTGGGCCATATCCTGTTGATGGGT
TTTGCCACCACTATAGAACCTTCTGTGGTCTCCCTTGCGAAAGGGGCACAGCTCGTCAGCGTATTTAAG
GAGTGACCAGAGTGGAGAGCATGAGGGGAACAAACAGGTGGGGGCTGTTATCCAGAGAACAGTGCTG
CTTTGTGCAAGAACTTCTAGGGTAGTATAGAATGGTGTTGGGAGCAAAGACTTTAGAGTGACAGAGAG
TCCTAACAGGTCTCTTCACTTCTCTTGGGCTGCAGGGATAATCCTACAGGTCCCTAATCCCTTATTTGA
AATCTTTTGGGATGGGTGTGTTTTGGGATTCAGGATGATTCAGATTTTAGAAAGGGAATATAGTGCCTT
TATGATACATAAAGTAATACCCCAGTGAGATTCANGGCAGTGCCCTACTGTATTCAAATCTATCAATG
GTTCTGTAGCTAAATATATGAATATTCCCACTTTAAGTGAGATAAATAGATTTTATAAATNGGGATCGG
GACCGGTGAAAACAAAATTTTGGGTCCAAAGTTNTGAACCAACTNTTGGTTTNTTCAAAAAATTTTTTA
TTTTGGNAATTTTANAAAANGGGATTATGGGCCTAAAAATTGGNCNCAAGGTTTTTGGGGGAGGAAAT
AAANTTTGGCCCAAAAAANCCTTTTTNAAAACNCCCCAATGGGNNGGNTTTTTTTNCCNATTTCNANNC
CCAAAANAAAAANCCAAAGGGCCCANGG
NNNTATCTGGAGCTNCANCGCNGTGGCGGCCGCTCTAGAACTAGTGGATCCCCCGGGCTGCAGGAATT
CGGCACGAGGTANNCTGCCNGNCNNCNTACTTGCNTGTNTNGGANGANGGNTNCGGGNNNCTGNGCC
AACNNAATAACNNTCGCATGGCTNATGACNGGTCATGATGCCTCAGANCCCGANGNGCGGGGATNCA
CGTGCGATCTTANGCCTTGTGNACGGGACGCCCGCCNATNANGATNCNACTTTCGCTCAAAAGANNGG
ATATTNTNTGCGCCNNNATCNTTTNCTGCTGCTAGAGAATCCTCAGGNAAANGGGNNGGNCGNCNTTC
ATANNCNACNNNNGAANANCCNNNTNCGCNGGNTNNNNNNGACCNCTTTGNGNTNCCCTNGNATNAAA
AGCNTNCGAGNGCAATATAANNCCCNTGTNCGNNAANNTGACNNCCTNGNGNNCNGCNTANNNGNG
NGNGNTNAACNTCNNACNGANAGNGNAAANTACCANANGGNGTNGATNCATNCCANNCCGGGANNT
GNTGGGCTTTGNACTATTTGGTGCCTGAANGCAANCGNCNNTCGGNNAGNGNCNAGANNNCAATCTNT
NAGGCGNNACTGGANGNACCTTTGACTGNACTCGGCGGACCAGACNTACANNNGAGGTNNTTGGGAG
ACGGACCTNTGCNTACTGNTTTCTNGNGATATAGNNTTGTGGATGAACGANTANTATNTNACGCCTTN
TACCTCTNTNGNATANGCGCGATGGATGNGNGTTCCCTTNACCTTCNCCTNCGGTNCNTGGGCTCGAG
CGCGGNCCNCTGGGCTTNTTTGCTTTGCTTGANGNGGGNCTCAAATCCCTTNTCGGACATTAAGGGGG
GGGNTTTANCGGTTCNTGGGGGAAAAAAAAGGNANTTGCCCCCTTGCTTCAANCGGTTTTTTAACCCT
TTTAANANTCCCANGGGAGGAACGCCCCCCATTTNANNCT
NAANACTGGAGCTCCANCGCNGTGGCGGCCGCTCTAGAACTAGTGGATCCCCCGGGCTGCAGGCCGG
CCCTGTCGGACCTGCCGGCAAGAGCGGTGATCGTGGTGAGACCGGTCCTGCTGGTCCTGCTGGTCCCA
TTGGCCCCGTTGGTGCCCGTGGCGCCGCTGGACCCCAAGGCCCCCGTGGTGACAAGGGTGAGACAGGC
GAACAGGGCGACAGAGGCATTAAGGGTGACCGTGGCTTCTCTGGTCTCCAGGGTCCCCCCGGCCCTCC
CGGCTCTCCTGGTGAGCAAGGTCCTTCCGGAGCCTCTGGTCGTGCTGGTCCCCGCGGTCCCCCTGGCTC
TGCTGGTTCTCCCGGCAAAGATGGACTCAATGGTCTCCCAGGCCCCATCGGTCCCCCTGGGCCTCGAG
GTCGCACTGGTGATGCTGGTCCTGCTGGTCCTCCCGGCCCTCCTGGACCCCCTGGTCCCCCAGGTCCTC
CCAGCGGCGGCTACGACTTGAGCTTCTGCCCCAGCCACCTCAAGAGAAGGCTCACGATGGTGGCCGCT
ACTACCGGGCTGATGATGCCAATGTGGTCCCGTGACCGTGACCTCGAGGTGGACACCACCCTCAAGAG
CCTGAGCCAGCAGATCGAGAACATTCCGGAGCCCTGAANGCAGCCCGCAAGAACCCCGNCCGCACGT
GCCGTNAACTCAAGATGTGGCCACTCTTGACTGGGANANCGGAGAATACTTGGGATTGACCCCCAACC
NANGNTTTGNAACCTGGATGNCAATTTAAGGGNTTNTGNCAACATGGAAACCGGNGGANAACTGTGTA
TAACCCCCCCTCAACCCCANNGTNGGNCCAAAAAAAAACTTGGTTTTNNTTANCCNNNAAACCCCCAAG
GAAAAAAAAGNCCNTTTTGGGTNCNGGNNAAAACATNAAACCGGNGGGANTTNNNNTTTNAATNTTG
GGGGGNCAAGGGGGCCCAANNCNTTNCNATNNGGNCNTTCNNANTTATTTTNNN
GCCCTATNTGGAGNTNNATNNCNGTGGCGGCCGCTCTAGAACTAGTGGATCCCCCGGGCTGCAGGTTA
AGGGTCACCGTGGCTTCTCTGGTCTCCAGGGTCCCCCCGGCCCTCCCGGCTCTCCTGGTGAGCAAGGTC
CTTCCGGAGCCTCTGGTCCTGCTGGTCCCCGCGGTCCCCCTGGCTCTGCTGGTTCTCCCGGCAAANATG
GACTCAATGGTCTCCCAAGCCCCATNGGTCCCCCTGGGCCTTGAGGNCGCACTGGNGATGCTGGTCCT
GNTGGTCCTNCCGGCCCTTCTGGACCCCCTGGTCCCCGANGNCCTNCCANCGGNGGNTACNANTTGAG
CTTCCTGCCCCANCCACCTNAAGANAAGGNTNACGANGGNGGCCGNTACTACCGGGCTGATGANGCC
AATGNGGNCCGTGANCGTGANCTTGAGGNGGACACCANCCTNAAGAACCTGAACCANCAGATNGAGA
ACATNCGGANCCCTGAAGGCAGCCGNAAGAACCCCGCCCGCACCTGCCGTGACCTGAAGATATGCCA
CTCTGACTGGAANAACGGAGAATACTGGATTGACCCCAACCAAGGCTGNCAACCTGGATGCCATTAA
ANGGTTTCTGTAACATGTGAAAACCGGGGAGACACTGTGTNTACCCCAACTTCAGANCCNAGAGTGGG
GCCANGAANAANATGNNNTATTNTNCANCNAAGNCGCANAGAAAAANANGGCACNTTTTTGTTACNG
GTGAGANCAANTGAACCGNCGGNAATTTCNAAATTTNTAGTATTGNNGGGCAGGGGTNCCCANACCN
TGNCGATGTGGGCANACAAGNNGAANTTTTNTNGCNCTTGANGNCCAGCNAGGNGCTTTCCCAAAAAA
TNNCNTACACTGGNAGAAACAAACGNGGCCTANTTGGGCCAACANNANTGNNGAACCCTTTAN
TTTGAGGCCNATAGNGAGTCGTATTACAGNGGCGGNCGCTCTATAACTAGTGGATCCCCGGGGCTGCA
GGCCCGGCGCTGTTGCAGNTNNGNAGCCCTGCGTCTTATCTNTCCAGNCGGGGTCCTTTGCCTCGCCAC
CCGCCCNTTCAACTGGCTGCGCCATGGAGGCNTACCATAAGTCTGATCAGCACAAGCTGNAGGCCNTG
ANGGACACAGGCCAANNGCNTGCGTATNANCNNCATCCAAGCCACGACTGGCGGNCAGNATNGNNCC
ANCCNACGTCATGCTGCAGCGCTGNTGAGATCATGGNTGTCCTNTTNTTACACACCATGCGCTACAAN
GNCCTGGATCGTCGGAACCCTCACAACGAGCGCTTTGTTCTCTGNAAGGGCCATGCAGGNAGCCGATC
CTTGTATGCCGTCAGGNGATNGAAAGCNTGNNTTTCCTGCCCGNAGTNANAAGCTGCTGGAATTTTGA
GGAAAGATCAGCTTCCNNCTTGGACGGGGCACCCNTGGTNCNTGAAAACANTGCCTTTTACCCGANTG
AGGCCANATGGGACTTCNTTGGNCCCANGGNCNTTGGAAGCCCGTTTTGCGGGNATGGCCTNCACCAC
GGCNATATACTTTNNACNANAGCCACGCNACCCGGGTTTTANTNGCNTGCCTNGGNATACNTGGCGAA
CTGGNCAACANGGACNTNCNNGNTGGNAAGGNCCANGGCCNTTCNNTCAGGCTNTTNNCNTANCTTN
GGANAAAATTTTGTTNGGNNATTCTTTGGAANATTCAACCCCCCNTGGGCCAANAATTGAAGCCTTNC
CCTCNGTTGTTGGNACCCANATTGGNCANTAAACCCACNAAGNGNCTTTCTAAGGNNCNTTTNNGGNN
TNGNNAANCGCCTTGNNCATTNANTNTNAATNGGGGACNNAANNGNGGNAAAGGAACNTNTGNCAN
GNGGGNNTTTTNGNCNAGGTGGNAANGATTCNGNNCCNATNGAGGCCTNTNTTTTNTGGGNCNAAACNN
TTTTNCAGGGGACCCNAGTGTNTTCNNCAATGGGNNNNAATACG
TTTGAGGCCNATAGNGAGTCGTATTACAGNGGCGGNCGCTCTATAACTAGTGGATCCCCCGGGCTGCA
GGCCCGGCGCTGTTGCAGNTNNGNAGCCCTGCGTCTTATCTNTCCAGNCGGGGTCCTTTGCCTCGCCAC
CCGCCCNTTCAACTGGCTGCGCCATGGAGGCNTACCATAAGTCTGATCAGCACAAGCTGNAGGCCNTG
ANGGACACAGGCCAANNGCNTGCGTATNANCNNCATCCAAGCCACCACTGGCGGNCAGNATNGNNCC
ANCCNACGTCATGCTGCAGCGCTGNTGAGATCATGGNTGTCCTNTTNTTACACACCATGCGCTACAAN
GNCCTGGATCCTCGGAACCCTCACAACGACCGCGTTTGTTCTCTGNAAGGGCCATGCAGGNACCCCATC
CTTGTATGCCGTCAGGNGATNGAAAGCNTGNTTTTTCCTGCCCGNAGTNANAAGCTGCTGGAATTTTGA
GGAAAGATCAGCTTCCNNCTTTGGACGGGGCACCCNTGGTNGNTGAAAACANTGCCTTTTACCCGANTG
AGGCCANATGGGACTTCNTTGGNCCCANGGNCNTTGGAAGCCCGTTTTGCGGGNATGGCGTNCACCAC
GGCNATATACTTTNNACNANAGCCACGCNACCCGGGTTTTANTNGCNTGCCTNGGNATACNTGGCGAA
CTGGNCAACANGGACNTNCNNGNTGGNAAGGNCCANGGCGNTTCNNTCAGGCTNTTNNCNTANGTTN
GGANAAAATTTTGTTNGGNNATTCTTTGGAANATTCAACCCCCCNTGGGCCAANAATTGAACCCTTNC
CCTCNGTTGTTGGNACCCANATTGGNCANTAAACCCACNAAGNGNCTTTCTAAGGNNCNTTTNNGGNN
TNGNNAANCGCCTTGNNCATTNANTNTNAATNGGGGACNNAANNGNGGNAAAGGAACNTNTGNCAN
GNGGGNNTTTTNGNCNAGGTGGNAANGATTCNGNNCCNATNGAGGCCTNTNTTNTGGGNCNAAACNN
TTTTNGAGGGGACCCNAGTGTNTTCNNCAATGGGNNNNAATACG
TTNANAAANGCTGGAGCTCCACCGCGGTGGCGGCCGGTCTAGAACTAGTGGATCCCCCGGGCTGCAGG
AATTCGGCACGAGGAGGTCGCGGAGGCAGAAGGCGTTCCTGCCCCTGTCCTCGTCACTATGTAGCTGA
GGGGCAGGAGGCCTGTTCCACGTTCTGGAAGGTTCTTGGGCTCGACCACGGCAGTAGCCCCAGGACTT
CTAGTCCTTGGCCTCGGATCCTTGCGGGCGGAGGGGAGTCGGCGCCGCCATGTTCGGCTGCTTGGNGG
CGGCCAGGCTGGTGCAAACAGCTGCACAGCAGGTGGCAAAGGATAAATTGGTTTTTGACTTGGCCTGA
TAATNAAAATATCACCCTTGTGGTGGTTTTAATGCTGGNACCAGTCCCTTTTCTGAAGGAANGGAAGC
TCTGGCTACTTTTCCTATCTGATTCAAANGAATGCCNGTATGGCAACTTCTAAGNATTGGCCACNATNG
GNAAACCAGTGGCCNTNTTNAAAATTCAGGTNTTAATTTNGGNAAGGAACCCACCNCCCGTTNGNACC
CTTNATATTGGCCNNACTTCNNTTNGTGCTTNAAATNGGAATTCAANNGNAATGGTNGCAAGGTTTG
TNAACAAANCTCTGGGGGGAAAGNTGGTGGGGCCCCNAGTGGACCCANTTACTTAATTTAACAAAAA
AAGCCNGGNCCACCTTTTAANAAATTTTGCTTTAATAANTTGGTNGGCCTTCAAGGNCCGANANTNAA
CCCCAAGTNCCCTTTTGNAAATGGTTNNTTTCCCNNCAAANNNGGGGNTNTNNAAAANGGGGNNNGA
AAAACNTTTTTAAAAAANNNNANTTTNNCNNNNAAACCCTTTTTTTTTTTGGNAAAACATAATTTTNN
NAAAAAAAAAAATTTTTTAAAGGGGNTNNTNNAAAATTTNGNNNTGTTTTNNAAAAAAAAATNNCTT
TGGGNNAAAACCCTTTAAANNNCNNAN
TTTNNANAAAGCTGGAGCTCCCCGCGGTGGCGGCCGCTCTAGAACTAGTGGATCCCCCGGGCTGCAGGA
ATTCGGCACGAGGGTCCTGTCCCTCTTGAATCTCCCTCCCACTTCCCCTTGCTACATTCTTGTTTCTATG
GATATATGAATAATAAAGACCGTTGACTAAAATAACATGAAAAGCTCCCACACACAAGTGTGCACGT
GCACACTCCCTTGTGCATTATTCGGGTCCACGAGAGGGCCCTGTCCCCGCAGATGCTGAAGTGATGGA
GCCTCTTGTCTCCGGAGCCAAGGGCATGAACACACTGTCGGTCGTCCTCTAGGGACACCTATGCTTCCG
CCTTCTGGCCTTTCCATTGCATCCGTCCGTTTCTCTAACAGAATGTCCCTGTGATGACACGGGCTCCCGT
TACCAGCTCTGGCTGTCTCAGTGAGAGTACAGCTTACCCTCAGCACTCTTTATTGGCCCTGGGAAATTT
TGCTTCAGTACTTTATTTGAAAGAAGCAATAAATAATACTGGGGATAAAAATAGTTCAAAAGTANACTG
GACATACTTTTGTCACATATTNGGGAAGTTNACAAAANAATTANTNGGAAAATNGAATGATACCATTG
AATTGTNCAAGGTAAATTAAAGGCGGAACCCCATGTAAAGANACATGCTGTCTTTTGTNTACCGAAAN
ACCAGCTGCATAATAAATTTTCGCNCCTTGACCTGGAAAAAAAAAAAAAAAAAAACTGNGGGGGGGGC
CCGGGNCCCAAATTCNCCNTAAAAGGGGGGNNNNNNNANAANNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNC
TNNNNAAAAGCTGGAGCTCCACCGCGGTGGCGGCCGCTCTAGAACTAGTGGATCCCCCGGGCTGCAG
GAATTCGGCACGAGGCTACACCAGAGGCGGCCGAATCATCTCCACCTCTTCACGCTCCGTGATGCTAC
ATTACCGCTCAAGCCTGCTCCAGATGCTCGACACGCTGGTCTTCTCGAGCCTCCTGCTGTTTGGCTTTG
CGGAGCAGAAGCAGCTCTTTGGAGGTGGAGCTATACCCAGAATACAGAGAGAACTCGTACGTGCCGAC
CACCGGAGGCATTATCGAGATCCACAGCAAGCGCATCCAGATGTACGGAGCCTACCTTCCGCATCCAC
GCCCACTTTCACTGGGCTCAAGGTACCTGCTGNACAACTTTCCCGATGACCTGCGCCTCGTTGGTGTC
GCCAGCAACTTTCACCTTCTTAAGCGTANTTGGGCTCTTAAGCTACATGCAGTGGGTGNGGGGGGGCA
TCTTGGCCCNACAGCGCTTTTTTTGNAGGTAACATTCCGAAACCGAAAACGTTCCCGCANGGACATT
CAAGCGAAAAGGGTCTNTGCCAATAANCCAGGGCCGCAAGGCCAGGANGAGTCCCCCCCAGCTTGTC
ACCTGGTTACAGAAGGACGGNGAANAGCCACGCCGGATCCCTTTTGGGAAAAAAAGGCCAGCTTGTC
CGAGGAGGGAAAAAACAGAACCAACCGGCCCCTTGAATGGGCAAGGAAGGAACTTGGAANCCCCAA
GGCCAAGTGATGGTTCAAGGCTCCTNGGNAANAAGCAACNTTTTGNTGAACGGAAGGCCAAACCTGG
GNTGGCTTTTGGGTTTTTGNCCCCTGGNCCCCAAAAAAACCGNGGGGCAAGCTTCGGAAANCCCCTTT
TGNGGGCTTTTTTNCCNAAAAGNGNCCCCAATTTTGGTTCCAAATTTCTTTNAAAACNANATAAGGGN
CCCAANCCCCCNATTTTTTNGGGCCCTTTTTNCCCCCCGGGTTTTNTTTTT
AAAGCTGGAGCTCCCCGCGGTGGCGGCCGCTCTAGAACTAGTGGATCCCCCGGGCTGCAGGTGATCTA
CAAATGTGGCGGGATCGACAAGAGAACAATTGAAAAGTTCGAGAAGGAGGCTGCCGAGATGGGAAA
GGGCTCCTTCAAATATGCCTGGGTCTTGGACAAACTTAAAGCTGAACGTGAGCGTGGTATCACCATTG
ATATCTCCCTGTGGAAATTTGAGACCAGCAAGTACTATGTTACGATCATTGATGCCCCAGGACACAGA
GACTTCATCAAAAACATGATTACAGGCACATCCCAGGCTGACTGTGCTGTCCTGATCGTTGCTGCTGGT
GTTTGGTGAATTTGAAGCCGGTATCTCCAAGAACGGGCAGACCCGTGAGCATGCCCTTTTGGCTTACAC
CCTGGGTGTGAAACAACTAATTGTTTGGCGTTAACAAAATGGATTGCACTGAGCCACCCTATAGCCAGA
AGAGATACGAAGAAATTGTTAAGGGAAGTCAGCACCTATATTAAGAAAATTGGCTACAACCCCGACA
CAGTAGCATTTGTGCCAATTTCTGGCTGGATGGTGACAACATGCTAGAACCAAGTGCTAATATGCCAT
GGTTGAAGGGATGGAAAGTCACCCGTAAGGACGGCAATGCCAGTGGAACCACCCTGCTTGAACTTCTG
GATTGNATTCTGGCACCACTTGNCCAACTGGACAAACCCTTGNGTTTGGCTTNTCCAGGATGTCTATNA
AAATGGGGGGGATTGGNACTGGCCCTTGNGGGTCGNGTGGAAAACTGGTGGTTTTCAAACCTGGCATG
GGGGGCANCCTTTGGTTCCAGNCAATGGGAACCAACTGGAANGGAAATCTTTNAAAAAGGCCCCCTT
GAANCNTTTGNGGGA
GCCTATAGNGAGNCGNATTACAGTGGCGGCCGCTCTAGAACTAGTGGATCCCCCGGGCTGCAGGTCTT
TTTTTTTTTTTTTTTNCAAAATTTCNNAAAGGGNNGGNTTNTTNTTNCCCNAANTTTTNNCNNCCCCAG
NCTNNNNGNNTTTTTTTTNAAAAANNTTNNCNTTNNNNTTAAAGGTTNGGNNTTTTTAAANNGNCN
GGAANCCTTNNCNNGGCNTNGGGGCCNTTTTTAANNAAAAAAGGGGGNNTTTNANNANAANCCNTGG
ANNNANCNCCCNNANNCCCAAAAAAANNNTNNNNGAANAAAGGGCTGAAANCCCCNGGNCAANGGG
GGNNNCCCCNTTTTNNGNGGGGNNNGGGGGGNNAANCCCNTNCCNCCNCCNAAANNGGGCTNAANTT
AAAACNTAAAAATTTNGGTTNGGGAACTTNNNNNTTAAATTTTNCAAANGGGNNNNGNCCCCNGNAN
NNAAAACCNTNGNAAAANNGGGCCCNNNGNNNTTTTTNNTAAANGNAAAAGGGCCCCTTNGNCCNNG
GGGGNNNNAAAAAAANNGGGGGGGTTNCAGNAAANAAAAAATTTNTTTTTNNCNGGGGGGGGGGGG
GGGGGGGNAAAAAAATAAAAAAAGNNTNNGGCCCCCTTTTTTTNTANNGGNCANNANNAAAAANGN
ANANTTGGGCCCNGGTNNAACCTGNCNNAAAAAANNGGGGGCTGGTTNACCCANNANNGGGGGGGG
NNAAAAAAAAACCCCNNNNTTTANTTAAAACCGNNNNTNTTTNANCCCCACCCCCCNNAAANCGGAA
ANNGGNNTCCCANANCNCGNCGNNGGTGGCCCCCCCCCCNAAAAGGGCCAAAATNNTNGAAAAAAC
CCCCCAACCCNNTAANANGGGGGGGNGCCCCNNNTCTTTTTTTTTTTAAAAAANGGTNGGGTAAAAGG
GGACNTTTTGNCCCGGGACACCNNGGAAACCCTTNNGGTTGTCCCCCCNTTTNTNNGNNGNT
CCNCCNNCAGGGNCNTTTNACNNNNANNTTTTTTNTTNTANCCCTNTCCNCNCTTTNTACCGGNNGCC
CCGNCTACTCCNCTTNNGGGGGTACCNGNCGNNGCNNCNCTNCNTTGAGNNNCACTGGACTNCACCG
CGGGGGCGGCCGCTCTAGAACTAGTGGATCCCCCGGGCTGCAGGCAATTCGGCACGAGGCTTGGTGAT
AAGGCTCCACCAAANCCTGTACCCAAGACTATTGACNACCANNGAGTATATNATGAAACCATAGTAC
ATCCTATTGATGAACAGGGTGCTTATGATGAANCTACANATNAATNTGCTNNTTACTTAAANACGACA
NACTTCTCCCAANATCTCTCATCNCTACNTCANCATANACCTCATGGGNANAACAGTACCNACTNTGN
GAACANGCTCTCTAGAGGTATACCNAANTTCGCATTGTTTATTAGCNNAACAANGACTTGCCTNTNG
AAAACNNNTTATTNCCACANNGCATNNTTATNNAAATTTNACNNACCNTGTTTTGNTNATNNAATNAA
AGANTCGTGAAAANTACCATAAANGGATTAAATTCATGANNNCTACCTGGTTTTCCAAACNNNNTCTNA
ANCNTGCGTTNCACATTATANNNAATTGANACCAANTGGNCCGNNCTNNCCNTTCAAANGAATNTTTA
NNCATNNANNGCCANATNNGACCNCNANNCNCNNAACNNCNCNTAACCNCNGAAAANNNANCNACN
TNNAANCNATCCTTCCCAANGCAACCNNCTAGTGNATCCANGTCCTNACTTCGGTAATCTTATANTNG
GAGNCACNTCANCTTGTTTTNNCCCANTNAATGNGCTNATAGNTNTNTAANTNGNNNNAGGCCTNNCT
TNTGNCCNATCNACTTTCTCNACNAATCNATACGGGANANTANATAATTNTTCTTNCNNANGAATCT
CCGNANCACATNCCTNTAATNTTCNAAGAATCCCNCGCATGAGANNNCGNGCGNANTTATTCGNATNA
TNTTTAGAANCCACNNGTNTTTGAACGNTTNAAGNNCATNGNACTNNAACNATNTCTANATAACNATA
CNNGGCCGATNNTNTTGNNTGCNTCTTCNTACNACNGTANTNGNAGAAAATTATGTANGANNGGTACG
NGCTANCNNAATATNCCTCNNGACGGGAGCTTAANTNTANTNTTANACANTGTTNTNCGAAGNTACNT
ACANTATCNACGCATTNTGTNGTCGTATNGNCCCAGGNTCTCTANGNNGAAACNTATGTCNNCNTNCG
CTANCNGCNNNNNNCACTCTCAGNTANCGCNCNTGNTCCGCTNTNTTNTNACNNANNCNCAACTATAA
NTGTNCNTTNGCNGTAACNCCAGTTCTCNNAACGTGTCNNGCNNANCATACGAAATATCGTTTTCGCTT
TTCNACNGAATGNCCNCTCTATCGNTACNATCCCC
NAANATCTGGAGCTCCACCGCGGTGGCGGCCGCTCTAGAACTAGTGGATCCCCCGGGCTGCAGGAATT
CGGCACGAGGGTGGTTCAGAGCTGCCTGCCCTAACTTCCCCTTCTTCAGCGTCTGTTTTCTCTCTCCTTC
CTGCTTGTACCTCCCCATGCTCCCTGCTCCCCCCTCTCCCTTCCTGACCAGGAAGGACATGAAGGGTTG
AATGCCAGGAACAAATGACAAGGAAGCCCTCAGGAGGATAGGGCCATCTGNGGGCTGANCCTTCTGN
CTCCCTNCANCTGNCCCGNCTNCAGGCCCTTAAANGGCACATNANGGGGGNGNGCTGNTGGGTGGGT
ATNCCACCTNCANCCCACANNACTCATTTGNAATTTTTATTATGCTGGAATANCTATTTTTGGTTCCTTT
TTTTTCTTTGGGGGGGGGNATGNGNGNGNGTATGNAAATATATAAATAGCGAGNTTCATTAAAACAGA
CTGNCCCACACCAAAAAAAAAAAAAAAAAAAAAAAAAAAAAAAAAAAAAAAAAAAANAANNNCNN
GGGGGGGGGGGGNNNCCCCCANTTNCNTNNTNNGGGGGGGGGNAAAAANNNNNNNNNNNNCNNNN
NNNNNNNNNNNNNNNTNTNNNNNNNNNNNNNNNTNTNNNTNTNNNNNNNNNNNNNNNNNNNNTNN
NNTNNNNNNTCNNNTNTNNNNNNNGNNNNNNNNNNNNNNNTNCNNNNNNNTNNNCNTNNNNNNNGN
NNNNNNNTNNNNNNNNNNNNNNCNNNNNNNNTNTNANNNNNTNTNNNNNNNNCNNNAGNNNNCNN
NNNNANNCNNNCNNCNNNNNNNNNNNNNNCNNNNNCNNNNNTNNNNANNNNNNNNNCNNNNNNNN
NNNNNNNTNCNNNNNNNTNNNNGNNTTNCNNNNNCNNNNNGNNNNNNCNNNNNTNCNNNNTNNCNN
TCC
TTTNAGCCCTATAGTGAGTCGTATTACAGTGGCGGCCCGCTCTAGAACTAGTGGGATCCCCCGGGCTG
CAGGCCGNCGNTGTTGCANNTCCGCAGCCNTGCGTTTNANCTTTCCAGTNTGNGTCCTTTGCNTNGCCA
CCCGCCNNTTNGCNTGGTTGCCCCTNGNAGGCNNAGNTTANNCNNNANAACAAAAANNNGNNGGCCT
TNAAGNANCCGGCCAACNGCTNGNTATTAANTTCATTCAAAGCCACCACGGCGGCNGNATCGGGCCA
CCCCACGTTATGCTGCAAGANGTGCTGATATNATGGCTGCTCTTTTTTTNCACACCATGCTGTANAGGG
CNGTGNATCCCNNNANCNCTCANAANNACCNTTNTNTTCTCTANAGGGNCCNTGNGNCNCNTATTNTG
TNTNCCGTNTGNTCTAAATNTGTNTNTNTGCCAGAACAGATCTGNTGNATNATGANGAATANCATCTN
CGACNTGGACGGGCACTNNTGTCCAANAAACAAGTCTTCACCGATGGNGNCNACTGGCTTNCTGGNCC
AGGGCCTTGNAGCCTNTTGNGNGATGGACTACACANGCNANTNCTTCCACAANAGCCACTACCGGGG
TCTATTGCTTGGTGGAAAGACGGGGAAGCTTGTAAGATNCCTCTGNGAGGNANANTGAATTTTCTTCA
GCATTTTNTCAACNGGGTGGAACCTTTNTTTGCCNNCTTTGNTTTGACAACCAACCGGNCNTGAAAAA
AGAACCNTNCCCTGNCTCGNTGGCCNAAANTNGAATGNGNAATCTANAAANNATTNNTTTGGCNATT
GCCTTTGGGAAGGTANCNCTTNTTTTTNANNGGNAGGGCCNCAAGNTNGNAAAGGANNCTTGGGCCN
TGGCCTTTTTNGGGANGGNAAAAAAAACCCGNCCCCCNTNTNTTTTATTTGGCCANAACCTTTCNNGG
GGCCCNNANTTTTAAAAGGAGATAGAN
TTTNAGCCCTATAGTGAGTCGTATTACAGTGGCGGCCCGCTCTAGAACTAGTGGGATCCCGGGGGCTG
CAGGCCGNCGNTGTTGCANNTCCGCAGCCNTGCGTTTNANCTTTCCAGTNTGNGTCCTTTGCNTNGCCA
CCCGCCNNTTNGCNTGGTTGCCCCTNGNAGGCNNACNTTTANNCNNNANAACAAAAANNNGNNGGCCT
TNAAGNANGCGGCCAACNGCTNGNTATTAANTTGATTCAAAGCCACCACGGCGGCNGNATCGGGCCA
CCCCACGTTATGCTGCAAGANGTGCTGATATNATGGCTGCTCTTTTTTTNCACACCATGCTCTANAGGG
CNGTGNATCCCNNNANCNCTCANAANNACCNTTNTNTTCTCTANAGGGNCCNTGNGNCNCNTATTNTG
TNTNCCGTNTGNTCTAAATNTGTNTNTNTGCCAGAACAGATCTGNTGNATNATGANGAATANCATCTN
CGACNTGGACGGGCACTNNTGTCCAANAAACAAGTGTTCACCGATGGNGNCNACTGGCTTNCTGGNCC
AGGGCCTTGNAGCCTNTTGNGNGATGGACTACACANGCNANTNCTTCCAGAANAGCCACTACCGGGG
TCTATTGCTTGCTGGAAAGACGGGGAAGCTTGTAAGATNCCTCTGNGAGGNANANTGAATTTTCTTCA
GCATTTTNTCAACNGGGTGGAACCTTTNTTTGCCNNCTTTGNTTTGACAACCAACCGGNCNTGAAAAA
AGAACCNTNCCCTGNCTCGNTGGCCNAAANTNGAATGNGNAATCTANAAANNATTNNTTTGGCNATT
GCCTTTGGGAAGGTANCNCTTNTTTTTNANNGGNAGGGCCNCAAGNTNGNAAAGGANNCTTGGGCCN
TGGCCTTTTTNGGGANGGNAAAAAAAACCCGNCCCCCNTNTNTTTTATTTGGCCANAACCTTTCNNGG
GGCCCNNANTTTTAAAAGGAGATAGAN
TNNNAAAAGCTGGAGCTCCACCGCGGTGGCGGCCGCTCTAGAACTAGTGGATCCCCCGGGCTGCAGG
CGGGCGCGGCAGGAGCTGACATGGACCCAAATCCTCGGGCAGCCTTGGAGCGCCAGCAGCTCCGGCT
TCGGGAGCGGCAGAAATTCTTCGAGGACATTTTACAGCCTGAGACGGAGTTTGTCTTCCCCCTGTCCCA
CCTGCATCCTGAGTCGCACAGACCCCCCATAGGTAGTATCTCATCCATGGAGGTGAATGTGGACACAC
TTGAGCAAGTGGAGCTTATCGAGCTGGGGGATNAAGATGGAGCGGATGTGTTCTTGCCTTGTGAAGAC
CNTCCACCAACTCCCCAAACGTTTGGGGTGGACNAGCGTCCCGAGGAGCCGNAGCTTGCCAGTGGCCC
ACGCCAGAACAGGAACGNACGTCTCGTAATTTCTTCTTTGTCTTCCAATTCTTCAACCAACCAAAAAAG
GGCCNATGCCAGTGGACGGCGGACCAAAACACGCCCCTTGGCACANTCTTGACAAGGAGAAGGATGG
AGGCCTTGATGGCGGGCAAAAGCCCGAANCTTGCAGTTAGCAGCGGNCCCCCACTTACAAATTGGGG
GAGCTGCCTCGTTTCCCTGGGAAACCCCAGGCCTGGCCANAGCCCTTTGGAAAAAAACCAAACTNTTT
AATGGCANTAGGCACCAAGGGGAAGNANAGCCGGCCCCCAATGGTGTGCCTTTCTTGGGAACCACCC
CCNGTTCTGGCTTTAATGGTAACCNTTCCCCTGGTGGGATCCTTTTTGGTATTNGNTAAAAAAATTTTG
GGAAATTAACTTGGGCCNTTTGGNAATNTAANTTTTGAAACAAAAANANGGNCCCCTTGNTTTTANGG
GGGGANAAAAAANTCCGNAACNCCCCGCCGGGNTTTGGGTTTAATGGGTGAAANGGGGGNCCCCCCG
NTTGGGGGAAATGGGAAAATTA
GAAAAANCTGGACTCCCCGCGGTGGCGGCCGCTCTAGAACTAGTGGATCCCCCGGGCTGCAGGNNNN
CGANNTCATGCTTNTNNTANTNTTTANNTNGANGGGGGNCNNGGTTCTGANGGNNNCCTANAGNGGG
GCGAANNNCATGAGCTCTAGCCNTGCCNTGANATGCCCATNCNTNAAAAAAANNATGTNNAACCAGN
CCCCANNGAAACTCTGNGNCCCNNNAAAANTTTTTNTTNTTTNNNCNGNCGTTTTAGGGNCACNTTTC
CCAANCACAGGGNTTTNNTNNNNNAAATNTCCATGCCCNTGGTTAAAAAAAANNNTTGGNNCNGATN
NTTNCCCCCAGNNCAAANATGGGNGNNCAGATNTNANAAAAAAANNTNTNTTGCTTGNGCCNCCANT
NNGNGNNNNNNTGGGNNGCTGTGCCCCCCACCCAGGGTNTTTGCCTNCCCTTTAGNAGNACACCCCAC
TGGACCTGTGCCCCCAACCACCNAACATNAACCCTCCAANGNAAAAAAAAAAAAAAAAAATTTTTTN
GGAACCCANGGGNGNTTTTTCANAAANNTNTTNAANNGGGNGGGGGGNCCCCCANTTTTNAAAAATT
TGGGGGCCTTTTTTTTCATTTTTTTTTCCAAAAAAAAAAAAAAANNAATTTTTTTTTTTTTTTCNTNAAA
NNAATTTNNAAAAAAAANGNGGGGGGGNGNNGGAAAACNCCGGGGANGGGGGNTTTTNNCCCCCAT
TTTTTTNGGGGNGGGGGGGNGGGGGAAAAAAAANNNCCNCCNGGGNTGTTNGAAACCCCCCCCCCN
CCNGGGGGGGGGGAANTTTTTTTTTGNGGGTNCCCACCCCATTTTTGGCCAAAANANAANTTTTTTTTA
ANAAAACTNTTTTTTTAAAAAAAAAANCCCCANTTTNCCCAAAANGGGGGGGCCNCCCCCAACGNNG
TTGGGGGGGGGGGTTTTTTNAANNAACNTTTTTTTTGGGGGGNN
NAAAAGCTGGAGCTCCACCGCGGTGGCGGCCGCTCTAGAACTAGTGGATCCCCCGGGCTGCAGGATA
AGAACTGACCTGTGATTNTTCTCTCCCTNCCCTCCCNCTGCANGTGTGGGTCCTAGGAAAGGACGTGG
NNCACCTTGGGTNGAAACTCAAAAAACTTTCCGANGCNCCACCATNAGCNGGNCCGAGCTGAACACT
CNCCCGTGTAATTTTAAACATCTAGCANTANATAATTGNGGTGGTGACATAAAGGACCCTGNTTCTGT
AGAGAGAAAAGCTTANCATAATTGTTGTGAAATGTAACATGANGCAAGTAACTNGNANTTTTTTTTGT
TNTTAAACATGTNNGNTTTTTTAAAATAGANTGATANAACTTTGCCAGNCTTTAAAAANCTTGGCTTAA
TTNANTATATTAATCTGTCCGTGCANAANTAAAACCANCCTTTAAAAATGTTAGGGGAATNAATTNGA
AGCTGGCGAATAGNAACTGATTTTAAGTTTGGTATTATGAAACATTNAAGGGCTTTTGTCCCCTAATTG
ATAATCATGTTTAANANGTAGTCATNTGCGGGTATAAGCTAGTGTTGCTTGCTTGCANTATTCATTGCC
CCCTANANGAAAGNGAAAATGGATTGGNATGGAACCCCCGAANTTAAAGTAAAAGAATGGNTGAAGT
TTTTTTTANAAATTTNCCCTTTCCAAANTGGAAAAGNNCCTTTTTAACTGGGCGGGTTAACAAATACCA
AAAANAANGNTGGNTTTTNTTGGCCCTTGGGAAAAAAATTTAATTTCCCCCCCCAGGTTTTTNNNNNC
NGGGGGGAAAAAGGGGGCCTTAAATNGGNAACTTAAGGGGTTCATGGCCNTCCNTTTAAANAANCCC
NGGGAACCGNNTNGGCCCTTCCAAACNTTTTTTNNNCCANGNGGGGTTTTTTANGGGGGGCTTTCCCC
CCGGGNGGNGAAACTCCCCAAAAAG
TGANAANNCTGGAGCTCCCCGCGGTGGCGGCCGCTCTAGAACTAGTGGATCCCCCGGGCTGCAGGAA
TTCGGCACGAGGCTACACCAGAGGCGGCCGAATCATCTCCACCTCTTCACGCTCCGTGATGCTACATT
ACCGCTCAAGCCTGCTCCAGATGCTCGACACGCTGGTCTTCTCGAGCCTCCTGCTGTTTGGCTTTGCGG
AGCAGAAGCAGCTCTTGGAGGTGGAGCTATACCCAGAATACAGAGAGAAGTCGTACGTGCCGACCAC
CGGAGCCATTATCGAGATCCACAGCAAGCGCATCCAGATGTACGGAGCCTACCTCCGCATCCACGCCC
ACTTCACTGGGCTCAGGTACCTGCTGTACAACTTCCCGATGACCTGCGCCTTCGTTGGTGTCGCCAGCA
ACTTCACCTTCCTCAGCGTCATTGTGCTCTTCAGCTACATGCAGTGGGTGTGGGGGGGCATCTGGCCCC
AACAGCGCCTCTCTTTGCAGGTAAACATCCGAAACCGAAAACGTTCCCGCAAGGACATCCAAGCGAA
AGGTCTCTGCCCATCAGCCAGGGGCCCAAGGCCAGGAGGAGTCCCCCCAGCTTGTGACCTGTTACAGA
GGACGGTGAGAGCCACGCGGATCCCTCTGGGACAGAAGGNCAGCTGTCCGAGGAGGAAAAAAACAG
AACAAACNGCCCCTGAATNGGCNAAGGANGAAACTTGGAACGCGGAAGGNCGAGTGATNGGGTTCAA
GGCTTCCTTGGGGAAAAATGCAACTTTTGGTTGAACGGGANGGCCAAACCTGGGCTTGGCCTTTTTGG
GCNTTTTGGCCCCTGGCCCCCAAAAANAACCCGNGGGGCNAGNTTTGGGNACCCCCTTTGGNGGGGCT
TTTGGTCCNGAAAAGGGGCCCCNATTTTGGGTTCCANTTTCCTTNAAACCAAATAAGGGCCCCAANTT
CCCCCNTT
GNCCTANTAGTGGNGTCGGTATTAACNGGGGGCATNCCNNATTTATCAANTTNTTTGTNTTTNNCNCAA
TCCNAGNNTCCNAACTTCGGCNCTCTNANGCNNTNTTCCCTACCCCCNTTCNCCNACANTNTCCTACNT
TCGTCNTAAATGGNGTCTCTTNTNGNCCGCNTACTCNNCATNCTCTCNTTNCNCTTNCCCCTCTCTTCNT
TCGTTTCACGGTCNCATCNNCNCTGGGNGTNGNNTATGAGNGNNNNTCANATTACTCTCTCNCNCGGA
GNNCCTCTACTTNCCNNNAGNNNNNCCTGCCCTCGATNNGTCTGTNNANTATNNCNCNNNNNNNCCTA
NTGACCCCAGNGNNCTNGCTGTCCNNTATCTNNNNANNANCNTNNTCANGNAGGGGNNNNNNTNTTN
GANCNGGAATTCGNNAGNNATNGATATNGNNTAGGNGNGCTTNGNACTCGNANGTAGTNGTNNGCNN
TNTGGNTNTAGAATNANAANNCNNNGGNATACNTNTGNNCTGATTTNNTGTCNGTCCNGCNNGTNAN
CNCTNNTNNNGGTNCATATNTNTTNTNCCGGTTGTNNGANGTNTNCNATTANTANNTCCNGNGNNAGN
NTTNNNTANACNNANGNATAATTNNGNNCGGGNTNACCANTNTNNNNCTNGNATCNCTAGNAGGAA
GGCTGNCGAANCGNTTNNNGNGGNCTGGNNGANNNTCNNNGGNGGNCCGNNNNNGNTNNNGCNNNG
TGTNCGNNNANTAANANTATNTNANNNATCNNNTNGTCNGCTGGGNNATANNTANCGACNTGTCNNT
ANCGCNANNNCGGNAANNGNGNCTNTTNTAATTNNTATNCAGNGCNCATCATGGNGNNATCAAAGAN
TCTNNCNTGNNNACCAGCANNCAGGGCGNGTNAGGCANTTGNCNNNTNAGTCCNNGTNCNNAGNTNG
CNTGAAGNNCAATTTGNNNTTNNCNCGNGCCCANAATTGNCCCCATNNTGNNNCTNTAGNCNNCTGN
NGCANCNNGNTNGCCNGANACCNTNCGTNNTGNCCACAANNTGTNNGGGTCTNNTAGCAGTGNCNNA
GCGCGCGGTACTGTATCGCGCNCANACGCGNCCGANGGACCNNCNNGTGTGCANTNACNANNTCTCN
NATNACTANNNAGNCGANANNGTANANAATGCGNNNANAGNNNACGAGATGTNCGCATNTNGAGNN
AGAGAACAGGGTGNCTGTNNGCGCGCGCGNGNGCNCACGCAGATCGTNATAGATATACATATANANN
NTCTTANCGGGCGCGATNANCGCGNNNCNCGACGNGCGCNNCGGCTNNCGCTGGANTGTCNNGNANN
TAGNTNGTCTCGNTTNCGTGTNNGACGNNGANGNANGGANTCATGNGNNTNTNGTTCGTCGNCCG
TTTGAGCANCATCTGGAGCTGCACCNCGGGGGCGGCCGCTCTAGAACTAGTGGATCCCCCGGGCTGCA
GGCACTCGCTCGCAGTTCGCTCCGCGCCCTCTGCAACCATGTCTGACAAACCCGATATGGCTGAGATT
GAGAAGTTCGATAAGTCGAAATTGAAGAAAACGGAANCGCAAGAGAANANNCNNNGTCTTCNNNAN
ANNNGACTGANCNAGNNACACTNTGTACNNTAGANNTAANGGANANTNCANTGTCNNAANNCCANCT
TGCTTTCNNCANNCNNGAGNNGNNTNTTTNNNNTNTCTNTCNCNTTTNCTNAAANNCAAGANGCCNTC
TTGCACGNGTGTTNNCTNGTNANNTTNGAGTNGCCNTNCCNNTNNTNTTGNCNTTTATNNNTNTTCCNT
TTGTTTANCNNGANAANNCCCNTCNTATNCTNANTNCACNCNTTNCNCNTTAGANNNNAGTNNGGNNT
CNAGATCAGCTTNNATANCNGANTCNNTGTANNNNNNNNTTCTTNNNNTGAGTNATCNTTNTNGTNNA
TTTNANNGANTGTTTTGANCAGNATNTTNANACNNNCGTTTACTCNNNCGCNGGAGNTTCNAGNTNTNNN
NGNGCTCTTNTNTTNNGACACANNTNANNNNCATGNCTTTGCNGTTGTATCTCNCGATNTTTNCNTANA
NTANNTNNAATGCATNNNGNCTGTTANNNCNANGCANTNGTTCTNTATCNNNAGNNANNTNGNTCNA
NGANTNCNTNNNANGTATTTATGGTNCNCNNCTGNNTCTTACNNTGACCNANTNGNNATTNNANNCTGN
NNNTCGCNNNTTTTTNTNGATTNCGGAGCACANTCTNCNGTNCNTNTNNNATNCAGTTTNNNAATNTCTG
TNTNNGCTNNTCGAGTCNANGTTGNTTTNTCCCNCNNTGTNCTCANCTTTGGANTNGTCNTATAATAAC
CTNTNNNTGTCNTNNTNAGGATNTANNCTANNTTTCTCNGTACTTNTCNTCTCNNTTNTCCCCTAANTTTT
NTANNCCTTTTCNNTGTTNNNCNATNNTTTCGNTCTNGGNGANCGANNACTATCATANGTNAGCTTTCN
CTATATNCNTCTACGTCG
NTATANTCTAGANGACNTNNACNANCNNNCNACTATNNTNCAANNCTCTCCNCACTNNTTTTNGAAGN
TACCCATTAACGAAGNTTCANCCNCTACAGANGGNCATTTCTTNTNCCNTCNTCNCTCATCACAGATNT
GCANNCNTACACTCCACNNNNTTNCTNNTNCNCNNANAACCNTNNNNCNNTTNTTTNACNTATTACTA
NCACNATTNCTACANATCANACGNTACCCGNGNNNNCTNCTACTTTCNCNNCTAATATTTANAANAC
ACTANCAATCAANTTATACTCCNTTTAANTTCANTATANTTNTTNANNNNCTCNNTCCCACACTACTTTC
TACNCNTCTTNCNANTNTTTAANNATTNCTTATTATATNANNCCCNAGTNTNCNACNTTNANNNTATAT
ATNTTTCTAAANNNCTANTNNGCCGNNNANACCNCAACCAACCTTNTTTTCTCNAATNGTCCTGCGCN
GNCNTNNNNCAANTTATANATNANACATNNNNANNNNNCANNCACGNNCTTACNNCTTTANTGNATA
TCTNNNNTTANCCCCNTANAGACTCANCNTATCANNCAATATTANATATCTANAAATATCTNNCATCNA
NTCATNTACCTTNNTAGNATCACTNTCNNNTTNTAATNTNNNTAATCTGNTNNTNANTNNTNTTNNATN
TANACTAATANAANCNNNCCACGACCNGCCAANTCNNTCTNTTNTNCNNCTACNNANNAGCTACTAN
CGNGTNCNNTCANGTNANCTTNNTTCTACNAATCANNTCNATNAANNCGACCCCTANCCNCTNCCNAN
NCTTGNCTCACCTTATNGCTATTCGCNCTNCCCNNCCTTNTNTNTTCTCACCTGTANCNTAGANGNNNA
NNCGNNNCCCTNACNTCTATANCCNTNNGTCTTTTANNNNANACTANNNNGTATCNNCAANNNATNA
NGTCTCTNTNGTCNTTCCTTCCGNCAACATATCANACNNNNNANATNCNNNCCNCTTCNANCNATTCC
AACTNTTTATTCTACATTATATTTANACCNNNNNTTCTCNCCTGTTACCTTNCCACCNTCTNNTNCCNTA
CTNCCNACCNATATGNCACCNGNCTCAANCATCTANNTACTTATANCATCTACAATNATNTNTANGAT
CGNCNNNTACNCNATNTATNCANNATACTTTCNCTNGANTNNNGCNNATNNNTTTCNTATTATTTTANN
TCTNCTCTNAANTTTANCCNTACAGNACNNCGAANNTNNNCATNTANTATCTTTCNNACANGTATTCAN
TCAGNACNATNTCTGGCTTTACATCANAATTCTATCAANATTNAATGCGCTNNNTTANNTANTCANNN
TCTTNCTGCACTGATNNTCTNTNTNTGACTANNNGCNNNATNTNTCNNTNTCTNTGAACNTACAAAT
ATATAAATNTGNNATCCTCNCNAGTNTATCNNTTTATNNANCNTATACACGCCTNCGANNCANNANTN
TCCATCTCANCTCATNTANATCTCTANAANAACNCAAAAATNTTTACATNTATNNNATANNTATNNAA
TANACATACGNTACATTATTNCNGCNATNNTATCNCNTNACACTACNANCATNCTAATCCTCTACGAC
GNCNTNACACCATACATNNGATATACNACAANATATATNACNAATCNNNAAACNATCNGCG
TTNAGCCTATNGGGAGNNGNATNACAGTGGGGGCCGCTCTAGAACTAGTGGGATCCCCCNGGCNGGN
NGGTTTTTTTTTTTTTTTTTTCAAANNAAANCCNNTTTTTTTNAANNNNNGGNNGGGNAAAANCCGGA
NNCCNNNNCCNNGTTTNGGNNCNNNNTTTAANNCCNAGGAAACNTTNNTTTCCNAAANNGNANNGG
GNAATNCCNAAANNAANNCNNGTTTTTTTTAAAANGNNNAANNACCCNGAAANCCTTTAAANNAAAA
NNNNTTTNNCTNAATTNAAAAAGNCCTTAATTTAAAAAAAANNGGGGGCCNTTNNAANNAAAANGGN
NNTCCCAAANNNNTTTTTNAAACNGNNNNCCNCCAAAAAAAAAATTCCGGGGGGGGGNNNCNTNGGG
ATTNGGCCCNNCCNNNNNNNGGNNAAANGGGGNNANNCNAACCCCGGGNNTTTGNTTTTTTTTCNAA
TNCCNAAANGGGAAANCCCTTTTNGGGAAGNNCAAAGGGGGGNGCCCCCCCCTCCCNAANNGTNCNG
NAAGGGCCCCTTTTNTTTTAAAAGGGTTAAAAACCCATTTTNGTTTCCGNCCCNTGGGGCAATGTGNG
GGGGAAAAANCCNAAANAANNNNNNTTTGGGGGCCCNNNCCCCGGGGAAAAAAAAACCCTTGCGAT
ATTTGGGGAAAAANNGGGGGGCTTAAAGGGGCCACCGGGGTTTTTNGGGGTTTNNCGGGGNGGCGCC
CCCCCCTTTTTTTTCTCTGGGGGGAAAAAAAAAAGGNNNAAAANCCNAGNCCCNTTTTTTTNNAATNN
NNCCCNNTTTANTTTTTNTTTTTTCCCCCCCCGGGGGGAAAAAANNTTTTCNCTGGGGTTGGGGGCCC
CCCNTTTTNGGGGGGGGAAAAAAANAAAGGGNGCCCCCCCCTTTTAAAGGGGNNCCCCCNNCCTNTTT
TTTTTTTTTTTTTTANAAGNGGGGGGGGGNTC
TTNAGCCTATNGGGAGNNGNATNACAGTGGCGGCCGCTCTAGAACTAGTGGGATCCCCGNGGCNGGN
NGGTTTTTTTTTTTTTTTTTTTCAAANNAAANCCNNTTTTTTTNAANNNNNGGNNGGGNAAAANCGGGA
NNCCNNNNCCNNGTTTNNGGNNCNNNNTTTAANNCCNAGGAAACNTTNNTTTCCNAAANNGNANNGG
GNAATNCCNAAANNAANNCNNGTTTTTTTTAAAANGNNNAANNACCCNGAAANCCTTTAAANNAAAA
NNNNTTTNNCTNAATTNAAAAAGNCCTTAATTTAAAAAAAANNGGGGGCCNTTNNAANNAAAANGGN
NNTCCCAAANNNNTTTTTNAAACNGNNNNCCNCCAAAAAAAAAATTCCGGGGGGGGGNNNCNTNGGG
ATTNGGCCCNNCCNNNNNNNGGNNAAANGGGGNNANNCNAACCCCGGGNNTTTGNTTTTTTTTCNAA
TNCCNAAANGGGAAANCCCTTTTNGGGAAGNNCAAAGGGGGGNGCCCGCCCCTCCCNAANNGTNCNG
NAAGGGCCCCTTTTNTTTTAAAAGGGTTAAAAACCCATTTTNGTTTCCGNCCCNTGGGCCAATGTGNG
GGGGAAAAANCCNAAANAANNNNNNTTTGGGGGCCCNNNCCCCGGGGAAAAAAAAACCCTTCCGAT
ATTTGGGGAAAAANNGGGGGGCTTAAAGGGGCCACCGGGGTTTTTNGGGGTTTNNCGGGGNGGCCCC
CCCCCCTTTTTTTTCTCTGGGGGGAAAAAAAAAAGGNNNAAAANCCNAGNCCCNTTTTTTTNNAATNN
NNCCCNNTTTANTTTTTTNTTTTTTCGCCCCCCGGGGGGAAAAAANNTTTTCNCTGGGGTTGGGGGCCC
CCCNTTTTNGGGGGGGGAAAAAAANAAAGGGNGCCCCCCCCTTTTAAAGGGGNNCCCCCNNCCTNTTT
TTTTTTTTTTTTTTANAAGNGGGGGGGGGNTC
TGAAAAAATCTGGAGCTCCACCGCGGGGGCGGCCGCTCTAGAACTAGTGGATCCCCCGGGCTGCAGG
NNNTCGCGNCNAGAGCTNACATGGACCCAAANCCTCNGNCNGNCTTGGNGCGCCAGNAGCTACNNNT
NNGGGAGCGGNACAAATTNTTNNANGACATTTTACANNNTGANACGGANTNTGTTTTNCCCCTGANCA
ACNTGNATCNNGANTNGTACANANCCCGCATNGNTANTATCTNATANNTGAATGTGAATGTCAACGC
ANTTNNNCAAGTGNANCTAATCGNANTGCTGGATCAATATAGAANGGATGTGATTNTGTCCTTGGGAA
CANCNTACANCNNNTNTNAAGANGGTTGGCCGNNANCAANCCNTNNTTAGGTTNNGACCTNTNNCAG
TTNTAANGCCAAAANACAAATATTTNTTGGGTCNTTNGNTNTTTGGGNNAACCCNCCCCCCGCCNAAA
NCCNAAAANNNNNNGCGTTTTTGNGNNGNNCNTGGTGANTTTANNTTCNNNGTTTTTGNAGGNCTTNT
TGNTTAGGAATGAANANATGGGNNCTCCCCTTNNNNNNTTTTGGCCATNANCCCCTCTAACCTTTTTCC
TCCCCCNCTCGCCGCCCCCTTTTTTNCCCCCCNTTTTTTTTTTTTTTTTCCCCCCCCTCNTCTCCNNCCCC
CTNAAGGGGGNGTTANTAANNNTTTTTTGCCCCNATTAACTNNCCNATTGGNTNNCTGGNGNAGGGNC
CCAATGTTANGGNNNATCTTTTTAAGGGGNANGCTTNGNNNANNANTGTAAATATAAAAGNTTGNAC
CNTCNNTTTNNTTAACAAATTTTTTGTCTTNATATTTTTTNNCCCCCCCCCCCCCCCCCCCNNTTATTTTT
NAAATTANATTANTATCATTATCTNNNTTNTTNNGGNGGGANCCTAGATNATNCATCCNNTTNCTTTTA
AATGGNGGGGGNANANNANAANCACNNANTANCCTCTTNTGGTTTNNTCCTTTCATTTTNANCTANTT
GNCTNNCAATNTTTTNCANTNTATTGTACCANCNT
TTNAAAAAACTGGACTCCCCGCGGTGGCGGCCGCTCTAGAACTAGTGGATCCCCCGGGCTGCAGGAAT
TCGGNACGAGGCCGGNTTTCGGATCGNGAGTNCGNGCCGNAGCGNGCACCGCGGNCCGNANCTATGGC
GGNNGTNGTCCGANTAGTTTATTGNGGCGCTTGAGGCTACAAGNCCAATTATNTTCAGCTNAAGAANA
AGTTAGAANATGAGTTCCCTANCCGNTTGGACNTCTGCNGAGAGGGGACTNCCCAGGNNAGCGGCTTC
TTTGAAGTGTNCGTANCGGNAAAGCTGNTTCACTCCAAGAAGGGAGGCGATGNNTACNGGGNCACGG
AAAGCAGGTTNCTGAGCTGGTGCCCGCNATCAANNCCGTTTTGACTCAGGCTNGATNTGGCCTAAAGG
NATAAACCAGTAATNGTGGCCCAACCCCTNTNGGNAGACCNTTATTTGACANGAAGGACTGAANTGTCT
TTTGGACNCNTGGTCCTTCCCTGATGTTTTGCCGGCCNCCGTTNGGAACAAANATGGACACCCCTGNN
CTTTTCCTTTGCATACCGGTGTCCATGAANACCCCCGACGCTGNGCCTGGTGGCCTTNCCCANANATAA
TTTGCNTTGATTTTCAACCGGCCCTTGCCCCAATGTTTNCCTTTTTAATCCTGGGGGGGGCTNGGGGGGG
GCCTACTNTAAGCTTTCNTTTTCAAAGGGGAANCTTGAAAGGATNATCNGGAATGAATGAGTCTTNAA
AATTTAANCAGNGGATGGNAACAACTTTACAAANCCAATNAAANGGGGGATGAAGGTTAACCCAAAA
AAAAAAAAAAAAAAAAAATTTNGGGGGGGGGGGCCCCGGNNCCCCAAATTNCCCCTTTAAGGGNGG
GNNNNNNNAAAANNNNNNNNNNNNNNNNNNNNNNNNNNNNNNNNNNNNNNNNNNNNNNNNNNNNNN
NNNNNNNNNNNNNNNNNNNN
TTNNNNAAAGCTGGAGCTCCACCGCGGTGGCGGCCGCTCTAGAACTAGTGGATCCCCCGGGCTGCAGG
CCGTGACAAAATGTGCTCCATGGTCAAAAAGTGGCAGACCATGATCGAAGCTCACGTTGATGTCAAGA
CTACCGATGGTTACTTGCTTCGTCTGTTCTGTGTGGGTTTTACTAAAAAGCGCAACAATCAGATTCGGA
AGAGCTCTTACGCCCAGCACCAGCAGGTGCGCCAGATCCGCAAGAAGATGATGGAAATCATGACCCG
AGAGGTGCAGACAAATGACTTGAAAGAGGTGGTCAATAAATTGATTCCAGATAGCATTGGAAAAGAC
ATAGAAAAGGCTTGCCAATCTATTTATCCACTCCATGATGTCTTCGTTTAGAAAAGTAAAAATGCTGAA
GAAGCCCAAATTTGAATTGGGAAAACTCATGGAGCTACATGGTGAAGGTAGTAGTTCTGGAAAAGCT
ACTGGGGATGAGACAGGTGCTAAAGTTGAACGAGCTGATGGATACGAGCCACCAGTCCAAGAATCGG
TTTAAAATGCAGACTCTTAATGGTGACAAATAAAAGATCTTATTTGTGGTNNNANAANNNNNNNNNN
NNNNNNNNNNNNNNNNNNNNNNNNNNNNNNNNNNNNNNNNNNNNNNNNNNNNNNNNNAANNGGG
GGGGGGGGGGCCCCCCCCCNTTTTTTTTTTTTNNGGGGGNNNNNNNNNNANAAANNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNN
TTGANGAANNCTGGAGCTCCCCGCGGGGGCGGCCGCTCTAGAACTAGTGGATCCCCCGGGCTGCAGG
ACNTNACNACGAGGGCTNCATGGTCANNANNCGGCANACCATGATNGANGCNCACGTNGNTGTCAAG
ACTACCGNTGGATACTTGNTTCNTCTGTNCTGGGTGGGNTTTACTAAAAAGCGCAACCTNATATTCNG
AAGACCTCTTACGCCCAGCACCNCCAGGTGCTCCAGATCCNCATGAAGATGATGGNAATCATGACCCN
ATAGGTGCATGACAAGTGACTTGAAAGAGGTGGTCANTACATTGATTCCATGATAGCATNGGAAAAG
ACATAGAACAGGCTTGCCANTNTATTTATNCACTCCATGATGNCTTGGTTNNAAAAGTANAAATGCTG
AAGAAGCCCAAATTTGAATNGNGAAAACTCATGGAGCTACATGGAGAAGGTATGNAGTTCTGGAAAA
GCTACTGGGGATGAGACAGGTGCTAAAGTTGAACCGAGCTGATGGATACGANCCACCNNTGCNNGAT
TCGGTTTAAAATGCAGACTCTTATGGGNGACAAATAACAAGATCTTTATTTGGGGTGGTNAAAGNTTNG
CTCGTATCTANAACAGTTTTAAAAAATGTACCANGNGNAATTTTATAGAATTTTGAAAGTTTACATTT
NTTTTTTTTNGGGGGNNGGCGCTCNNNCCACCCTTTNTNNTNAATTGTTNTNAAAGTGGCCTTTTCCCA
AACCTNGCTTNAAAACATTNGTTTTTTTTAATANNACNTTAAAAAAAAAAAAATNCCTTTTNTTTTTTA
AGNNNTNNTNNNCTNNNTCNNNNNTTTNNTNTCNTTTNNTNTGNGGGGGGGGNCCCCNCNNNNCCCN
ATTTTNNCNTTTTTTTNNNTGNNTTCCTTNNAAAANNCNNNNNNNNTNNNNNNNTTNNNNNNNTNCC
TNNNTNNNNTNTTNTTNTCNNNTCTNNCNNNNCN
NNCNNGCNNAACCANTCTTTTNTNNTNNNNNTNNNNNNNNNNTNNCCCCCCNNCTTCNCCCNCCCCCC
TCNCNTAANCTTCCACANATAANGGNNNANCNTANNNTTCGTTCNNCCCTTTGNAGNCCCACTGGAGC
TCCNCGCGGNGCCGGCCGCTCTACAACTANTGGATCCCCCGNACTGCAGGNAATTCGGCACGAGGGA
GTTTTTTTTTTNTTTTTNCCCCCCCCCTNCTCNNCGTNCCTNCCCNTAANNACTTTTNTTNANCCCNCTC
NTANCTTTTCCACNNCNACCTNCCNCTNTTTNCTACNANCCCNNANTCCNCCCANTCATCTNAANCCTT
CTNTCTGTTNCATNNATATNNTNTCTACACCCANAAAANCAAATNNNCCTNANGACNTCTNTTCCNNN
NNCNAAANNNNTANCAANNNNNTNCTNNNNNNANNNNAAAGGATANTANNAAACANANANNGTTAA
NNNCNCTTTCNNTTTNTCNNNGGTNTANNTAAANCCATTACTGANATCGANTCTCTTANTNTACNGGT
NNGANTCNNANTANTCNNNATNAANTCCNANNANCNTATACNTNNNCTTTCTNACTAATCNNNTAGTAN
NNATNCTNNTNCNNNNNCNNGNTNTGTNCNNNNAANNATNNNANTNNATCCANNNNGTTTNCNCCNT
CCTANGCNTNCTNANCNNANNNTNTTCNNAANNNTGANATCNNCCNCNCNATNNCTNTANCCCCCTCT
CNNNAANCAGTNGNCNNGNNNTNTAAAATATANAACCCATCNNNACNACNTNTTNCTANTACNACNG
GNACTCANGGTCNNNATNANCATTAAATCCGNTANTNTNNCNNCGNCNNNNACNNANTATTNNNNCTT
ATCCCAANATNCNCGGANNTTNNTTAGNTNNNNTTNTNTNANCGCTNTATCNCTNCCGNTACTCNAAG
TCNTNNACTGCATNTAGGATCNNTCGNGNNCNNTCTNTCNNCCACCGTNNANNTNNCNNNCTTCATAN
GTGNGCGANTACGAGNNNCCGNATNNNATCNCNCNTATANNATATACTTCGCTTTCNNCATCGCNNCT
NTCANNNTCNACAGTCTACCCAGNCTGCATGNCGTCTTACTCTGTNCNTCTCNCACACNNTNNCCGNN
ANNNTNNNNACATNACTCTNNNNGCTATATCACACCGTNNNANGNACNTATNCNANATNTCCNGANA
NTNCACNCCTAATANTCNACNGNGATNTATCAATCGTCATNCTCTCNCATNTNTANTCTGATTANTTNN
TCNCTCACACNNATNNTAGCANTCTTNNAGAGTNTNNNNTNACNNTNACACNANCCTCNCTATAGTNT
TNGATACTCCTTNAGCNCGNNNCCTCTTNACNCTCGACGCGTCGTATANNTCACNNCNANTCNNATGN
NNNACATGTCACGTCTCATCNNACACATTTGCGGNATNATCTCCAANANATNCNNAACGTCNNNCNAT
TTATNTCTNCGCN
AAAGCTGGAGCTCCCCGCGGTGGGGGCCGCTCTAGAACTAGTGGATCCCCCGGGCTGCAGGCACTCGC
TCGCAGTTCGCTCCGCGCCCTCTGCAACCATGTCTGACAAACCCGATATGGCTGAGATTGAGAAGTTC
GATAAGTCGAAATTGAAGAAAACGGAAACGCAAGAGAAAAATCCACTGCCTTCGAAAGAAACGATTG
AACAGGAGAAGCAAGCAGGCGAGTCGTAATGAAGCGTGCGCCGCCAGTATGCACTGTACATTCCACA
AGCATTGCCTTCTTATTTTACTTCTTTTAGCTGTTTAACTTTGTAAGATGCAAAGAGGTTGGATCGAGTT
TAAATGACTGTGCTACCCCTTTTCACATCAAAGAATGGAGAACTACTGACAACGTAGGCCGCGCCTGC
CTCTCCCATCTGCTTGTGTGGCTGGCAGGGAAGGAAAAGAACTTGGATGTTGGTGAAGGAGGAAGCTG
GGTGGGACGACAGTGAAATCTAGAGTAAAAAGCTGGTCCAAGGTGTTCTGCGGGCTGTAAAATGCAG
TTTAATCAGAAGTGCCATTTTTTTTTGTTGGTCAAATGATTTTAATTATTGGAATGCCCAATTTTTTTAT
TATGCAAAATAAAAAGTTTTAAAACCTGAAAAAAAAAAAAAAAAAACCTNGGGGGGGGGGCCCGGN
CCCCATTTCCCCTTTTNGGGGGGGNNNNNNNNAANNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNN
GTGNAAGGGGGGNNCGGGTGTNGGCCTTTCTTNNNTNGANNCNATCNGGAGCTNCACCNCGGGGGCG
GCCGCTCTAGCAACTAGCGNATCCCCCGGGCTGCTAGGCNTGNANCAGATNTTGANTNTTATTCATCT
TTTTCNNNTACCCTNCTTTTNTTNNTCTGGNGNACTAAAAGGCCCNNAANNACNANGTTTTATNATAAN
NNCANGANCTGATTACCATNTATANANANCNGCAACNGCGANACNCCCNTTGANGAAACAAGGAGGC
GACCAACAAGCNGATTTNCCCGGTTATTATTNNCNCCTGCGNNTTANTAAGGNTTTNTCCTANCTCGG
GNNCGCCCNTNTNAGNAACNTATTCNCCCAGCCCTANANATGNNCCGATANTNNTNATACTCGACTTA
ACCNNAGNANNTCTNCGTNNTGTTNACGNNGNNNANACNNANTGAACTNCCTGTNAACANTGGNGNT
CNAAGNNCNATAGNCNTGAATTNATCTCNCCACNTGCCTNNNNGNGGNCANNNCNNTNAANCNGTTT
NTATATNTNGATATGCNGTTTGCATNTCTNNCNAAANNNGGANTCNTANNANACCGCNTACNTANNG
NGCTATCTACCCCCNANGTCNTNTCNCNNCCAAGAGNNTNNNTCTANTCTACTCGAANGNATNCCCNA
NGANCGNTTATCNNACTCTCCNCCTTGNCGTNNTCNCTGATTCACGNCATNANTTNCNNNTANNTCTG
NCCCANCTACCAAATGNTGNATAGCTTTTNTTTNNGGACNNATTTCACTNGTACCCNNNATNTGGAN
NCTNNCCCCNCCTTCCCANCTNATGCCCNATATACCGTCATCCNTNNTAATTCGNNNNACACCNACA
NNAGCNCCNNGTTCTTNNCNACTNTCNTTTTTTTCCNACNNNTNGTCGNNGNNNGCNCTTNNCTNGCG
NTTNNGANTCAACTNGCCNGNGCTTNNNCGTNTATNANTCNTNTANNCCTTCNNTGNACGGCGGGCGN
NTNTTAGNNTCTTCNNACTNGNTNAGTNNAAGCTNATTAGCTNNTNNTAACCGATNNANNACTATNTN
ATTNTACNTCTGTCNNCNTCGACTATNCACANTGCTACNNNGCNTAACTCNTTNNTATANTGNNGNTT
CNNAGCTAGNCTCTAACTTNTGATGTANCGNGTNNNNANCGCTCNNGGGTGATTTTACNCANATCNNN
AANTATTCTCTCGCGATNANNNCNNGTTCNCGTNNGNNNGATNTGNNTCTTANAGNCTNATNAGCACT
NTCTAAANNTNANACNTGANCG
GCCTATNNGGAGCTNCATCNCNGTGGCGGCCGCTCTAGAACTAGTGGATCCCCCGGGCTGCAGGCANN
TGTNNAGGGGGCACAGCAAGAGCCGCCCCCAGGAACCACAGACCCAGTAGAGACCTGGCTGTCCCCT
TGGGCATGGCCTCTCTGCCCTCTGGACCTGTCTTCCATCAGGAGAAACCACTGAGATGGGTCGCCTCC
CCCAATGCCACATACCACAAGGACAGGGCTCATAGGCCCTCTTCCTTGCCAATCTGATTCAGGGCCTC
GGGTGGAGTGAGGAAGGGAGGCCCCACCCCCCCAGCCTTCAGCGTCAAGCTGGGCAGACCCAGGAGG
TGTGCCCCCGATGAGGACCCAGCTGCGGTGACCCCTCTGCCCCCTTCTACCTCGCCATCAGCTCCCTAC
CCAGCTTATCAGCTGGGTCTCACCTGCCACCCTCTTGCCTGGCCAGTGGGAAGTGCAGGGAGATGGGA
GGAGGACCTAGCACACAAGTAGGCCCTCAATAAAAGCTGGCTGGTGTTGCACTTTAAAAAAAAAAAA
AAAAAANTAANGGGGGGGCCCGGNCCCCANTTNCNCCNTNAAGGGGNGTNCGAATAACATTTGNTNN
TNTTNTNNNNNTNNNTTCGTNNNNACCNATNCNNTCNNNNNTTCNNTCCCNTNNNNNNTNGNNTCTACTT
CTCTNTATNTCTGTNNNTNNTNTTNCNNTTTTNCTNTTCNCCNATTNNNTGNTTNTTNNTNTTTNNNGN
GNTCTNNGNTNTTATNNGNNTTGTCCTNNAACNTTNCNTNNCNCNTAGNGGANCCCTTGNCCNNNGGN
NTNTAATTCANNTATTNCTNTNNCNNANTCNTCTTNNNNNNNCTTCNCNNATTNTNNTNNTTTNNCNNN
NTCANTNTTNNNCNCTNNTNNCNCTTNNCTNNNTNTCTCNNNTTTTNNNNGNGNNCNGNTCATTNCNA
CNNNNNNNNTTCNACGTGTCGNGGGTGCCC
GCCTATNNGGAGCTNCATCNCNGTGGCGGCCGCTCTAGAACTAGTGGATCCCCCGGGCTGCAGGCANN
TGTNNAGGGGGCACAGCAAGAGCCGCCCCCAGGAACCACAGACCCAGTAGAGACCTGGCTGTCCCCT
TGGGCATGGCCTCTCTGCCCTCTGGACCTGTCTTCCATCAGGAGAAACCACTGAGATGGGTCCCCTTCC
CCCAATGCCACATACCACAAGGACAGGGCTCATAGGCCCTCTTCTTCCTTGCCAATCTGATTCAGGGCCTC
GGGTGGAGTGAGGAAGGGAGGCCCCACCCCCCCAGCCTTCAGCGTCAAGCTGGGCAGACCCAGGAGG
TGTGCCCCCGATGAGGACCCAGCTGCGGTGACCCCTCTGCCCCCTTCTACCTCGCCATCAGCTCCCTAC
CCAGCTTATCAGCTGGGTCTCACCTGCCACCCTCTTGCCTGGCCAGTGGGAAGTGCAGGGAGATGGGA
GGAGGACCTAGCACACAAGTAGGCCCTGAATAAAAGCTGGCTGGTGTTGCACTTTAAAAAAAAAAAA
AAAAAANTAANGGGGGGGCCCGGNCCCCANTTNCNCCNTNAAGGGGNGTNCGAATAACATTTGNTNN
TNTTNTNNNNNTNNNTCGTNNNNACCNATNCNNTCNNNNNTTCNNTCCCNNNNNNTNGNNTCTACTT
CTCNTATNTCTGTNNNTNNTNTTNCNNTTTTNCTNTTCNCCNATTNNTGNTTNTTNNTNTTTNNNGN
GNTCTNNGNTNTTTATNNGNNTTGTCCTNNAACNTTNCNTNNCNCNTAGNGGANCCCTTGNCCNNNGGN
NTNTAATTCANNTATTNCTNTNNCNNNNANTCNTCTTNNNNNNNCTTCNCNNATTNTNNTTTNNCNNN
NTCANTNTTNNNCNCTNNTNNCNCTTNNCTNNNTNTCTCNNNTTTTNNNNGNGNNCNGNTCATTNCNA
CNNNNNNNNTTCNACGTGTCGNGGGTGCCC
TGANAANATCTGGAGCTCCACCGCGGNGGCGGCCGCTCTAGAACTAGTGGATCCCCCGGGCTGCAGG
AATTCGGCACGAGGGTNACGTTGGTGCTCCCGNACCCAAAGGTGCTCGNGGCAGNNCTNGTCCCCCTG
GNGCTACTGGTTTCCCAGGTGCTGCTGGCCGAGTCGGTCCCCCCGGCGCCTCTGGAAATGCTGGACCC
CCTGGCCCTCCTGGCCCTGCTGGCAAAGAAGGCAGCAAANGCCCCCGCGNNGAGACTGGCCCCGCTG
GGCGTNCCGGTGAAGTCGANCCCCCTGNTCCCCCTGGCCCCGCTGNTGAGAAAGGAGCCCATGNNGCT
GACGGACCTTGCTTGAANTTCTTGGCACTCCTGGACCTCAAGGTTTTGCTGAACAGCGTGGNGTGGNC
GNCCTTTCTTGGTNAAAAAGGAAAAAGAGGCTTCCCTTGGTCNTTCCTGGGCCCCTTTGGTGAAACCC
GCCAAACNANGTNCTTTTTGAACCAGNNGGAGAAACGTNGCCCCCCTTGGTNCCAATGGGCCCCCCCT
TGGATTGGCTTGGAACCCCCCTTGGATAANNCTTGGACCGTTAAGGGAACTTCCTTGGTGNTTTAAAN
GAATTNCCCTTGGAAAAAAATGGTTNTTTTTTNNCCCCCCAANGGGNNANCCNNNGGGNGAAAAACC
GNTCCTTTGTTGNAACCNNCTTTGCNCCTTTTTGNNCGCTTTNCCGGGGGGGCCCCNGGGCCNTTNTTG
GNACTTNTCNGGCNCCCNCCCGGCCCCCCCCCCCCCCCNGAAACGGGGCCTGNTTGGTNCCTTTNTTTNG
ANCCCANTTGGGCCCCGTTNGGTTNNCCCCNGGNCCCCCCCTTNGAAANCCCAAGGCCCCCCTNNGGN
NANAAAGGGTTNNNAANAANGCAAANAANGGCCTAACNAAGGCNTTTNNGGGNTCNCCCGGGGANT
TTTTTNGGNTTTTAAGGGTTCCCCCCGGCCTTTNCGGGTTTTTTTGGGCNCCCCCCCCCCCCCCCCCCCC
CCTTT
ANAAAACTGGACTCCCCGCGGTGGCGGCCGCTCTAGAACTAGTGGATCCCCCGGGCTGCAGGNNNGG
TCTGGGNGNCNNGTGCTCCAAGGAAGTAGCCACTGCCATCCGNGGGGCCATCATCCTGGCTAAGNTGT
CCATCGTCCCTGTGCGAAGAGGCTACTGGGGGAACAAGATCGGTNAGCCCNACANNGNTCCTTNNNA
NGGGACTGGCCGATGGANATCNTTNCTGNTGCGCCTCATCCCTGCCCCCAGAGGCACTGGGATCGTCT
CCGNCCCTGTGCCCAAAAAANTGNTNNTGATGNNCGGATTNGNCGACTGNTNCNCTTCTGCCAGGNGC
TGNACTGCNNCCCTGNGNAANTTCANCNANGCCACTTTTGATGCCNTTTNCAAGACCTATNGNTACCT
NAGCCCTNAGGTNTGNAAAGAGACGGTNTTCACCAAGNCTCCATATCANGAATTCACTGACCATCTTG
NGAAGACCCACACCAGAGTCTCCGTGCAGAGGACACAAGCCCCAGNTGTAACCNCCANATAATTTTAT
AAAGGGAATAATAANGTGANTGAAACCNTGTGNNNGNAGTGNNGNNTNNANGTTGGGGGGGGGCCC
NGCCCATTTTGACNCTNAGGGGGGCTTNAAAAANNNNNNGCNNANAGGAACTGTNGCCACGGGTGCG
GGGTGNGNGNTGAACCCTGNCANCATNCCTTTNGAGGTGGNANCNCCANCNCATNGGNAANCCTTTT
ATTNTTNNCAAAAACCCCCTTCTNGCCNGAAANNGGGTTTTAATTNNTNCCCNGCGGAGANGCCNTTT
NCGGGGAACCAAAAATTTCNNGAAAAAGNAAANNNNGGNNTNGGNNTNAANAAANNTTNTTTTTTTT
TNCAAAAAAAAAAAAAAAAAAANTNNGGGGGGGNCN
NNACAGCTGGAGCTCCACCGCGGTGGCGGCCGCTCTAGAACTAGTGGATCCCCCGGGCTGCAGGAATT
CGGCACGAGGATGGCTTTAGGGATGGAGATGCTGCTTGAGGAGCCCGCAGATAAGTTTTTTCTATTAAA
AAAGATAGGCGTTAGTGTATTTCTTTAGGGATTATAAGTAGTGAAAATACGGAGATACTGCTTTGCAT
TAAGTTCTTAGATAAATTTTTAGGGAAAATATGAAGACTTAAAGAGTAGTTGGAGAAAGGAAGAGGGA
AGAAAAAAGATAATTTTAGAACCTGAGGGAGGTTAAGAGCTGAAATTTTTTCATGGAGTGAAAAATTT
AAAAGAATTGATGAGCTAGGACTGCAATGTCTGGATTTTTTTTTTAAAGCCTTAACTTGAAGGGCAAC
CTTTTACCTTAACATTAAAACGTTGGGTTACGTAAATGGAACCTAANCGTTTTAAAAGGTTGAGTTGTA
GGTGATTAAAATACTACAAAGGCCATCTTAANATTTACCTGAAGGNGGTTTCAGAAATAATGGCTTCA
NATTCNCGGCGCAGTAAAAATGGCGTCATTTTGAAGCCTGCTTGATACTAAGGCTTTGTTTGGCTAAA
GTTGACCGGAAATATTAGGAATGGGAGGGAAAGAAAATTGGAAAGTTTGAAGTGGGAAAATTTCGGA
AAAACAGAAGTNCCGGGAAGGNGGAANAAAAAACCTTGGAGAAGAATTTNGGAGGTTCCCCGTTTTAA
AAAAAAAAAACCTGGGCTTACGGGAACCCGGNCCCAAAAANAACCTTCGNNAAAAAAAATTGGGCN
AAATTTCCCCAGNGGGCCTTCAANANCTCANGGGGTTTCCCAAGNCNGGGGCCAAANAAAAAATNGG
CNGGGTTNTTTTTTCNNNNNNNNNNNNNNNNNNNNNNNNNNNNNNNNNNNNNNNNNNNNNNNNNNN
NNNNNNNNNNNANNNCNNCCCCN
TNNANCAAAGCTGGAGCTCCACCGCGGTGGCGGCCGCTCTAGAACTAGTGGATCCCCCGGGCTGCAG
GAATTCGGCACGAGGGAGAAGGGTTAATTTTCCTGAGTTTGGATAGTTTTTGAGCGCTTGACTTTAATTT
CATTGCGACCACTCAAATCTCTTAGCACATTGCTTAGGATTAAGGATCCAAAAATGCTATCTAGAGAG
TTGCTAGTGGTGTTGAACAATGCAAGTTTTTATTTAAAAGAGCTCTGCACTGCCATTTATGAAAGCCTC
TTTATGATGTTTGTTTTGTTGTCATTTTTGTTCTTTACACCAAGAAATTGTATGTACAAATATGCAGAGA
ACATATATTGCCTCTGCTTTTATCAGGGCGCTCACCCCTGGTACTTCGTATATAAAATGTATTAAAACT
GGGATTTGTTACCAGTTGCTGTATTTTGTATATAGAATTTTTATAAATTGTATGCTTCAGAAATAATTTA
TTTTTTAAAAATAAATTAAAAAATTTAAATCCACATCCATGGTACACCTTTCTTGCCTGAAATGTAAAG
AATCCATTTGTCAGCAGGGATCCAGAACCCCATCAATTGTGAAGTTTGCTCTATCTAGAACAGTTTAAA
AATGTCAGTGTATTTTATAGATTTGAAGTTAACATTCTTATTTTCAAGANAATTTATGAACATTGTAGA
AATGTATAAATGCATTTNCCAACTGGCTTAAACATTGGANTTTTATAGACTTAAAAAAAAATNCTATG
TTTAAGTNNNNNNNNNNNNNNNNNNNNNNCCCTCGGGGGGGGGGCCCGGACCCCAATTNCCCTTTT
AGGGGGGGNNNNTAANNNNNNNNNNNNNNNNNNNNNNNNNNNNNNNNNNNNNNNNNNNNNNNNN
NNNNN
AAAGCTGGAGCTCCCCGCGGTGGCGGCCGCTCTAGAACTAGTGGATCCCCCGGGCTGCAGGAATTCGG
CACGAGGGAGNTTTTTTTTTTTTTTTTTNNNCCNNCTTNAATTTTNNTTTNGNTTTTTTNAANGNNGCCN
NNNCCCAANTTTNTTTCNTTNGCNTNNTTTNTTTNATTTATTTNNTTCNNNNNCGNNNNTTTNTTNGAT
CTNCCTAAAANAAANNGGGNACCTTNGGGNCCNTTTTTTTTTAANNNNAANNCNANGGNCNCCNTAA
CCNTNTNAAATTNGGAANNTTTAACCCNNCCCAAAGGGNAAGGGGGTTTNCCNAAAANCCGTTNGGGN
AAANNAAAGGGTGNTTTTNTAANCCNCCCCNNANGGGGNGNNACNNANNATNGATGCCCCAGGACAC
AGAGACTTCATCAAAAACATGATTACAGGCACATCCCAGGCTGACTGTGCTGTCGTGATCGTTGCTGC
TGGTGTTGGTGAATTTGAAGCCGGTATCTCCAAGAACGGGCAGACCCGTGAGCATGCCCTTTTGGCTT
ACACCCTGGGTGTGAAACAACTAATTGNTGGCGTTAACAAAATGGATTCCACTGANCCGCGTATANCC
AGAAGAGATACNAANAAATTGNTAAGGAAGNCANCACCTATATTAANAAAATTGGCTACAACCCCGA
CNCAGNANCATTNGNGCCAATTTCTGGCTGGAATGGNGACAACATGCTANAACCAANGCTAATATGC
CNTGGNNCAAGGGANGGAAAGNCCGCCNNNGGACNGNANGCCNNNGGACCNCCCTGCTGGAACNNT
GGNATNGNNTNNGCCNCNANTNNNCCCACTNGANAACCCTTGCGNNNGNCNNTCCAGGANGNNNNTA
AAANNGGNGGNNNNGGNCNNGNCCCNGNGGNNNNNNNNGNNNNCNGNNNNNCNNNCCCNGNNNNG
GNGGNNNCCNNNNNNNNNNNNNNNNN
NCANCAAANCTGGAGCTCCACCGCGGTGGCGGCCGCTCTAGAACTAGTGGATCCCCCGGGCTGCAGGCG
CCATGATCGCCTTTGTCCGCCTGCCCAGCCTCAAGGTCTCCTGCCTGCTTCTCTCAGGGCTTCTCATCTA
CGATGTCTTTTGGGTGTTTTCTCCGCCTACATCTTTAATAGCAACGTCATGGTGAAGGTGGCCACGCA
GCCGGCTGACAATCCCCTTGACGTCCTATCCCGGAAACTCCACCTGGGGCCCAACGTTTGGGCGCGANG
TCCCGCGCCTATCTCTGNCTGGAAAACTGGTCTTNCCTANCTNCACGGGCAGCCACTTNTNTATGTTGG
GCATNGGANACATCGTGATGCCTGGACTCCTGNTGNGCTTTGNCCTGCGTTATGACAACTACNAGAAG
CAAGCCAGCGGTGACTCCTGCGGTGCCTNCGGACCCGCCAACATNTNCGGGCGCATGCAGAAGGNTTC
CTACTTTCATTGCACCCTCATTGGATACTTTGGAGGNCTGCTCAGTGCGACTGTGGGCGTCTTGAATTC
ACCGANCANANCCAANCCTGGCCTTCTCTATTTGGTGCCATTTACNCTTATTGCCACTCCTCACGAAAG
GCCTATTTAAAGGGNGACACTACGGCGAATGTGGGGGCTTGAGCCATTTNCACTCCAANATCCACCAA
GATCCCCGATTCCTGGGAAGTATGATGGATNACAGCGGGGCGANCNAGAAANGGGGCCTCTCANATC
CNTTTTCTGTTNAAACGTGGTGGGNTTTGTTTNCCTTCTTAAAAGNTGGNCCCTGGNTAACTTCCGGAA
NANGCANCCTGTGTTAAAAGAACTTGCCGTGGTGCCCCNNAATTTTNCCATTNTTANGGAAAGCGAAG
GTGGCTNGGAAACCCCTGCNNGGNTTNCCCTTTCTTNTTACCTNGCCGGCCGGNAAAAGGGGNNAGCC
NCCCTCCNTCG
TTNAGCNANATCTGGAGCTCCACCGCGGTGGCGGCCGCTCTAGAACTAGTGGGATCCCCCNGGCTTGN
NGGNAGNTGTCCAGGGGGCANAGAAGAACCCGCCCCNAGGAANCNNAAANCCNANAAAAANCTGGN
TNGNCCCTTGGGCNTNGGCNTTTTNGCCTTTGGANCTNGCTTNCATTAAGNAAAACCNNTNNAAAGGG
NNCCCTTTCCCCAATGNCNCATNCCANAAGGNCCGGGNTTATNAGCCCTTTTTCTTNGCAATNTGANTC
AAGGCCTTNGGTGGANTGANGAAAGGANGGCCCACCCCCCCAGCCTTTAACGTNAAGCTGTGCACAC
ACCANGAGGTGTGCGCCCCGATNAGAGACACACACTGTGGNGACACCTCTGTNCCCTTTTANNTCTCC
ATANAGATCNCTANNCAGNTTATANGNTGGGTCTCACNTGNCACCCTCTTNNCTGNGCACNGGGAAAT
GCNCGGAGATGGGAGGAGGACCTATCACACANTAGGCCCTCAATAAAANCTGGNTGGTGGTGCACTT
TAAAAAAAAAAAAAAAAAACTCNTNNGGGGGGGCCNGGGNNCCAATTTNCCTTTAAGGNGGGGGTTTAA
ANNNNNNNNNNNTNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNTNNNNNNNNNNNNNNNNNNNNNNN
NNNNNNNNNNNNNNNNNNNNNNNNNCN
TTNAGCNANATCTGGAGCTCCACCGCGGTGGCGGCCGCTCTAGAACTAGTGGGATCCCCCNGGCTTGN
NGGNAGNTGTCCAGGGGGCANAGAAGAACCCGCCCCNAGGAANCNNAAANCCNANAAAAANCTGGN
TNGNGCCTTGGGGNTNGGCNTTTTTNGCCTTTGGANCTNGCTTNCATTAAGNAAAACCNNTNNAAAGGG
NNCCCTTTCCCCAATGNCNCATNCCANAAGGNCCGGGNTTATNAGCCCTTTTTCTTNGCAATNTGANTC
AAGGCCTTNGGTGGANTGANGAAAGGANGGCCCACCCCCCCAGCCTTTAACGTNAAGGTGTGCACAC
ACCANGAGGTGTGCGCCCCGATNAGAGACACACACTGTGGNGACACCTCTGTNCCCTTTTANNTCTCC
ATANAGATCNCTANNCAGNTTATANGNTGGGTCTCAGNTGNGAGCCTCTTNNCTGNGCACNGGGAAAT
GCNCGGAGATGGGAGGAGGACCTATCACACANTAGGCCCTCAATAAAANCTGGNTGGTGGTGCACTT
TAAAAAAAAAAAAAAAAAACTCNNGGGGGGGCCNGGGNNCCAATTTNCCTTTAAGGNGGGGGTTTAA
ANNNNNNNNNNNTNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNTNNNNNNNNNNNNNNNNNNNNNNN
NNNNNNNNNNNNNNNNNNNNNNNNNCN
TTGANAANNTCTGGAGCTCCACCGCGGTGGGGGCCGCTCTAGAACTAGTGGATCCCCCGGGCTGCAGG
AATTCGGCACGAGGANAGCGGANAAGCCCAGCCGANAACTTTCANGGACNCTGTGAAGCAGCTCCAG
CCGAGATGTGCNAGGAGGAGGACAGCACCGNCTTTGGTGTGTGACAATGGCTCTGGGCTCTGTAAGGC
CGGCTTTGCGGGGGATGATNCTCCCAGGCCTGTTTTCCCATCTATCGTGGAACGTCCTCGACNTCAGGG
AGTGATGGTGGGAATGGGACANAANGACAGCTACTTGGGTGATGAANCACAAAGCAAAAGAGGAAT
CCTGACCCTGAAGTACCCGATAGAGCACGGGCATCATCACCAACTGGGGACGACATGGAAAAGATCT
GGCACCACTCTTTTTTACAATGAGCCTTTGNGGTGGNCCCTGAANAAGCATCCCAACCNTTNTAACCG
AAGGCGCCCCTTGACCCCCAAGGCCAACCCGGGAGAAAATGACCCAGATNATGGTTTGAGACTTTCAA
TGTCCCANCCATGTATGTGGCTATTTCANGCGGNGCTTGCCCCTNTACGCCTTTTTGGACGCACAAATTGG
CNNTTGNGCCTGGACTTTTGAANATGGGGTCACCCACAACGTGCCCCTTTCTTTTTAAGGGGTACCCCCCT
TGCCCCCCCNCCATTATTGCCGTTTGGGACCCTGGCTTGGCCCGAAANTNTGANCCCGNCTACCCTTAT
TGAAAAANTNCTTGACCCGAACCGANGGCTTTTTTCTTTTNNGAACCCCCCCNTNGACCGNTGAAAAA
TTGGTGCCGGGGACCTTTAAAGGGGAAAAAACTTGNGGCTTACCNTAANCCCCCTGGGANNTTTTTAA
AAANTGANANAGGGGCCNCTTTGNCCGNATTNNTTTTTTCNTTTCCCNTNTNAAAAAAAANTTTNTTTA
AACTTTNCNTTNNGGGNCCCGGGTGATTNNCCNTTTTNGGAAAAAGAACGGTTTTCCCCTTTTCCCCTT
TANAAACCNT
TTNANCAAAGCTGGAGCTCCCCGCGGTGGCGGCCGCTCTAGAACTAGTGGATCCGCCGGGCTGCAGGA
ATTCGGCACGAGGGCCATGGGCCGCGTGATTCGTGGGCAGAGGAAGGGCGCCGGCTCCGTGTTCCGC
GCACATGTGAAGCACAGAAAAGGCGCCGCGCGGCTACGCGCCGTGGATTTCGCCGAGCGACACGGAT
ATATCAAGGGCATCGTGAAGGACATCATCCACGACCCGGGCCGCGGAGCGCCCCTTGCCAAAGTGGTT
TTCCGGGATCCGTACCGTTTTAAGAAGCGGACAGAGCTGTTTCATCGCTGCTGAGGGCATCCACACCGG
CCAGTTTGTGTACTGGGGCAAGAAGGCCCAGCTCAACATCGGGAACGTGCTCCCGGTGGGCACCATGC
CTGAGGGCACCATCGTGTGTTGTCTGGAGGANAAGCCTGGTGACCGAGGCAAGCTGCAAGAGCCTNT
GGAAACTATGCCACAGTCATNTNCCACAACCCTTGANACAAANAAAACGCGAGTGAAGCTGCCTTCN
GGCTCCAAAAAGGTCATNTTCTTTTGCCAACANAACTGTGGTCGTTGTGGTGGCTGGANGTGGCCNCA
TTGACAAANCCATTNTGAAGGCCGGGCGNGCCTCCCCAAGNTTAAGGGNAAAAGGAACTTGGTGGNC
NCNGGTGCNGGGNGNGGNCATNAAACCTTGGNAGCCNTTCCTTTGGNNGGGGNAACCNCAACCAATT
NGGGAAANCCNTTTTTNTTTTCNAAAAAANCCCCTTTTTGGCCGGNAAANNGGGNNTTANTTTTTTNCC
CCCNGGAAAGGGGCNTTTTCNGGGGAAACCAAAAATTTGCCCGGGGAAAAGGGNAAACTTNGGGGTT
NGGGGTTNAANNAAAAGCTTTTTTTTTTTTTTTCNAAAAAAAAAAAAAAAAAAAAACTTTGGGGGGGG
GGN
TTNNANAAAAGCTGGAGCTCCACCGCGGTGGCGGCCGCTCTAGAACTAGTGGATCCCCCGGGCTGCAG
GGGAGACGCGAGCAGCTGTGTCTCCGGCAACTGAGGTAGAGGTTTAACTGTCGTCCCGGCGGAGCAA
GTCAAGACACATCTACGGCCGTCTGGGCCAGTGTTGGGTCTCCGTAGCCTGTAAAGATGGTGTTGCTC
ACGATGATCGCCCGAGTGGCGGACGGGCTCCCGTTGGCCGCCTCGATGCAGGAGGACGAACAGTCTG
GCCGGGACCTTCAACAGTACCAGAGTCAGGCTAAGCAGCTCTTTCGAAAGTTGAATGAACAGTCCCCT
ACCAGATGCACCTTGGAAGCTGGTGCCATGACTTTTCATTACATTATTGAACAAGGAGTATGTTATTT
GTTCTGTGTGAAGCTGCCTTCCCTAAGAAGCTGGCTTTTGGCTACCTAGAAGATTTACACTCAGAATTT
GATGAAGAGCATGGGAAGAAGGTGCCAACTGTGTCTAGACCCTACTCCTTTATTGAGTTTGATACCTA
CATTCAGAAAACCAAGAAGCTTTACATTGACAGTCGTGCTCGGAGAAACCTAGGCTTCATCAACACTG
AACTGCAAGATGTGCAGAAGAATCATGGTGGCGAATATTTGAAGAAGTGTTACAGCGGGGAAGAAAC
CGCTTTTCAAGCANTGGATTTCGAAGGCTAACAATTTTGTCCAGTCTGGTNCCAAAAAAATACCCGNC
AGGGATGCCAAAGTACNTTGAAACATGCCGNTTCACNTTACCNCCCAAACNTTGGCAGGAGGAAGCT
GGGANTTTTTTCATCATGGGTAAAAAAGGGGATGNGNCCCCTTNTTGGGGGGCNTGGGNAANTAATG
NAATNCCCGTCCCCTGGGCAAGGGGANAAACCTTNAAAACCCCGGNGNGGGGTNTNNTTTTAAGGNA
AACTTGANCCCCCCAAAAAATTNTNTNTT
NNCAAANCTGGAGGTCCCCGCGGTGGCGGCCGCTCTAGAACTAGTGGATCCCCCGGGCTGCAGGAATT
CGGCACGAGGATTTATAAGTTACAATGGAAGGCAACTTCATGAATTAGATGGGCTACTAATTCAATGTA
ATGAACCACACACCAAAATAAACAGTAGAAGGTGCTTGTAATTGTTGAGAGAAATTAACAAAGTAGA
ACCATGTTGTTGTTCAGTCGCTAAGTAGTGTGCAACTCTTTGCAACCCCATGGGCTGAAACACATCAGG
CTCCTCTGTCCTCCACTATCTCCTGGAGTTTGCTTAAATTCATGTGTATTTGAGTTAGTGATGCCATCTAA
CCATCTCATCCTCTGTTGTCCCCTTCTCCTTTTGCATTCAGTCTTTCACAGCATCAGGGTCTTTTCCAGT
GAGTAGGCTCTTTGCATCGGGTGGCCAAAGTATTGGAGCTTCAGCAGAAACATTCGATCCTTAAACTG
AGTACTGTCTTAGTTTCCTCGGGCTGCCTTAACAAAGCACTACATACTTTAAACCAACATACTACATAC
GTTAAACCAACAGGCAGGGTTGTTCCCTTCAGAGGGCCATGAGGGGGAATCTGTCCCANGCCTGTCTC
CTAACTTCTCTTGGTCTCCTGGTCATCTTTGACTGGCCNTGGCATGTAAATATCTCACTCCAGNGCTCTG
NCTTNCTTTGACATTCTCTCAAGTTTCTTCACATAAGTGTACGTGNCATGGATGGTGTNTTTTTGGGGGN
CCAGNTTTCTTTCCTTTTTATAAAGGACTNCAATCCTGGTNGGATGAAGGCCNCCCCGTTTTGANCCAT
CATGGACTTTGGATACCCTTGGGAAAAGGGCCGGANTTTTCCNAAAAAAAAANCCCCCTTTTTGANGGT
TNTTGGGGGGTTAAGGNCAC
NNNATCTGGAGCTCCACCGCGGTGGCGGCCGCTCTAGAACTAGTGGATCCCCCGGGCTGCAGGAATTC
GGCACGAGGGTTGGCTTCGTATCGTGGGCTCGTCCGCTGTTACCCACCTACCAGAGCCCTTCCCCGCGG
GGCGGGACCTCGTAAGGGCCAACTCCTTTCCAGCTGAAGAAACACTTAAGTTGTGGAAATAGCGACGC
AGTATCATGGCTGGCAGCCCTAATGAAGATTCAGAAGGAAGCCGAATTACTTACGTGAAAGGAGACC
TATTGCATGTCCCCAAACAGACTCTTTAGTGCATTGTATCAGTGAGGACTGTCGAATGGGCGCTGGG
ATAGCTGTCCTCTTCAAGAAGAAATTTGGAGGAGTGCAGGAACTGTTGAATCAACAGAAAAAGTCTGG
AGAAGTGGCTGTTCTGAAGAGAGATGGGCGATATATATATTACCTGATTACAAAGAAAAGGGCTTCAG
ACAAGCCAACTTATGAAAACTTACGGAAGAGTTTAGAGGCTATGAAGTCCCATTGTCTGAAGAATGGA
GTCACCCGACCTTTCCATGCCAAGGATCGGATGTGGTCTTGATCGTCTGCAATGGGAAAATTTATCCGC
AATAATTGAGGAGGTCTTTGAGGCAACAGACATCAGAATTACTGNGTACACACTCTGAACAGAAGAG
CATTTTGGATGTGTCTACCTTCTGCATCACCTGGGGCACAGGACTGGAGCAAACTGACCTTAAAATGGT
CAGAGGACCCGTTTCTTGNGAAGTAGTCTGGGTTACANGNCNAACCTGGGTTGCTGTATTCNTCANGA
TTGAATGGGACTTNTGGGAGGAGGGGTTCTTGTGCTTAGGAATTTTTNTTTTTCAGGAANGGAANGGG
GANGGGACCAATNTTGGGCCNAGGACCCCTTTTTGCTNGGGAANGCCTTGGGAATGGGTTTGGGTTGC
CCTAGGGGGGANCNTTGGGTTTTCCTGNTNNT
AAANCTGGAGCTCCACCGCGGTGGCGGCCGCTCTAGAACTAGTGGATCCCCCGGGCTGCAGGAATTCG
GCACGAGGCTGATTTTTTTTTTTTTTTTTNGTTTTAATGGNCAAGGNGNNTAANTTNANAAANNANNNC
CNGNCNNGNCCNTNTGTNNAAAGCCCNTTAANNATTTTNTTNATNTAATNTGGATNNNTTAACGGTTN
NAANGGNCNTTTAAAACTNTCCNTAAANTTCNNTNAGGAAAAAGGGANTTTGNNANTNTGNGNGNGC
CNGGGNCAAANGGNAAAAAACCAAAAAAAAANCCCCAGGGNAANGGGAAATCCCNTAACNTNAAAN
CATTTNNNAAAAGGGCTTNNGANGTTTTTTNNAAANAGGGCCTNNANNAGGGAANCCANTTTTTTTTG
ACCNNNTTNTNCNNNGCCAANAANAAAGGGAGTNTTTTNAANTAACCNTTCCCAGTTNANGGACCAG
CCCAGNATTCCTCATTNCAAGCCCTCNATNAGNCCCTGNGGGACCCAAGGGGCAAAACCTGTGTTGGG
AAAAGGTTTCNTAGGTTGGGAAAGGCCCAAAGCATTTTTNCCCCCCATTCCAGNNGGGTTAGGACCCC
CCCCCCAATNTTTAACAATGGNCCCANCCCGTTTTTTTCCGGGNATGCCCCNNCGNGNCCCNAAATGAN
CCTGCACCGGGGGGGGCCCGGGNCCCCAATTCNCCCTTAAAGGGGAGGNNNNNTNAAANNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNC
CAAAAGGTGGAGCTCCACCGCGGTGGCGGCCGCTCTAGAACTAGTGGATCCCCCGGGCTGCAGGAATT
CGGCACGAGGGTCCCGTATGTGGCCCAAGAGATCCAGGAGGAAATCGATGAACTGCTTCAAGAACAG
CGAGCCGACATGGACCAGTACGGCAGCATCAGCCATGGCATCGTGGAGGTCGACCCGATGCTCACCCC
AGAAGAGCGTCACCTGAGCAAGATGCAGAACCATGGCTACGAAAACCCAACCTAGAAATACCTGGAG
CAGATGCAGATTTAAGCGGGGGAAGCGCANCGCCAGTGGAGGGAGGNTGGGCGGGCAGCAGACGTC
CAGAGGTCTGGATGGACGATGGACCGACAGTGACTTCCAGAGGAGTTCCTCGACACTGAGATCTCCTG
GAACATTAAGACTATNAAGTACTACTGTAGAGTCACAATTTCCATTCTTTTAAATGGGCGAAAAAATG
TTAATATAACAATATATGATATATAAACCTTAAATGAACAAATGATCTATTGCAGAAAAAAAAAAANN
ANANAAANNNAANAAAAAAAAAAAAAAANNTNNNGGGGGGGGGGGNGGGGNCCCAANTNCCCNNA
ATGGGGGGGGGTNTTANANNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNTTNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNC
TNAGNCCTATAGTGANGTNGNATTNCAGTGGCGGNCCGCTCTAGAACTAGTGGGATCCCCCGGCNTGN
AGNANTTCGGCACGAGGNTGANTCNTATTNTNTTNCGCTTTTCTACGGCCNNNGANNCCNTTTTNNN
ANTNNNTCCNCCCNNCTGGCAAATNNCNGGGNAAACCTTTAAANATTTNTTTTNNTTNGGNTNGGCCC
TTNAGGCTNGTNANGGNNTTNNAAAANCNCCCTTCANNTTNCTTGGGNGNAANNGGNNNAATGCANC
TNTTNNCGGNGAAGGGGNTAAACGGNACTNACGCCNAAAANCATGNAAACGNGGCCTCTCNCTCGTC
TANTNTNNAAACAATTNTTCTNNAGGNCTNTTNNTTTTTNTCACAAAAAGGGGTNTNACAGAGGGAGN
CCCCATTTTTTNNNTNNAAGATTTTNTGNGCCCANAANANGANNGGGNNTTTTTATAANNGCNNTACC
TGTGTGTGGAAGACCCCCCNANANGNCTTTTTTTTTAAAGNAGGTGNNTNGGANCNCNTNTNTGGAGA
NACGANNGAGGAAAAACNANGNTTCCNCAAAAAGNTTTTTNTTTNTCGNNGNAANATCTCCCTTTTNT
ACTCTTTTNCCCCNCANNCTGTCNGNGNTNNTTNNNACCCCCCCCCAANTTTTTTTANAAANATACCNC
NCCTCTTTTTTANTTTCNTGGGGAGGAACNNCCCCCCCCCCCANCATGGAANCNTCCCNAGGTNNGGG
GGGGNCNNACCCCCCNNNCACTTGGTTTTATTTTTANAAGNGNNGCTATGANNNNCANNTANNGNTNT
CGTTTTNTNTNNAACNCNAATCTTTNAATTNTCNCNCCCCNNNNCTNGCNTCTGGTCNNTTANTCTANC
CTCATNNCCTTCCNGNTNNANTNNNTTCNCCCANTNNANTTTNCCGACCTANNNAAANAANNTCTNTN
NCCTTCNCAAGAAANGNTAATTTCCTTCNNNANNCNCTGAATTCNNCTTAATNNTCGNCNNTNTNTAT
TTNCCG
TNAGNCCTATAGTGANGTNGNATTNCAGTGGCGGNCCGCTCTAGAACTAGTGGGATCCCCCGGCNTGN
AGNANTTCGGCACGAGGNTGANTCNTATTNNTTNTTNCGCTTTCTACGGCCNNNGANNCCNTTTTNNN
ANTNNNTCCNCCCNNCTGGCAAATNNCNGGGNAAACCTTTAAANATTTNTTTTNNTTTNGGNTNGGCCC
TTNAGGCTNGTNANGGNNTTNNAAAANCNCCCTTCANNTTNCTTGGGNGNAANNGGNNNAATGCANC
TNTTNNCGGNGAAGGGGNTAAACGGNACTNACGCCNAAAANCATGNAAACGNGGGCTCTCNCTGCTC
TANTNTNNAAACAATTNTTCTNNAGGNCTNTTNNTTTTTNTCACAAAAAGGGGTNTNACAGAGGGAGN
CCCCATTTTTTNNTNNAAGATTTTNTGNGCCCANAANANGANNGGGNNTTTTTATAANNGCNNTACC
TGTGTGTGGAAGACCCCCCNANANGNCTTTTTTTTTAAAGNAGGTGNNTNGGANCNGNTNTNTGGAGA
NACGANNGAGGAAAAACNANGNTTCCNCAAAAAGNTTTTTNTTTNTCGNNGNAANATCTCCCTTTTNT
ACTCTTTTNCCCCNCANNCTGTCNGNGNTNNTTNNNACCCCCCCCCAANTTTTTTTANAAANATACCNC
NCCTCTTTTTTANTTTCNTGGGGAGGAACNNCCCCCCCCCCCANCATGGAANCNTCCCNAGGTNNGGG
GGGGNCNNACCCCCCNNNCACTTGGTTTTATTTTTANAAGNGNNGCTATGANNNNCANNTANNGNTNT
CGTTTTNTNTNNAACNCNAATCTTTNAATTNTCNCNCCCCNNNNCTNGCNTCTGGTCNNTTANTCTANC
CTCATNNCCTTTCCNGNTNNANTNNNTTCNCCCANTNNANTTTNCCGACCTANNNAAANAANNTCTNTN
NCGTTCNCAAGAAANGNTAATTTCCTTCNNNANNCNCTGAATTCNNCTTAATNNTCGNCNNTNTNTAT
TTNCCG
TTGANNAACCGNTGGAGCTCCACCGCGGTGGCGGCCGCTCTAGAACTAGTGGATCCCCGGGGCTGCAG
GAATTCGGCACGAGGCTCACCATCAACCCCCAAAGCTGAAGTTCTATTTAAACTATTCCCTGAACACT
ATTAATATAGTTCCATAAATACAAAGAGCCTTTATCAGTATTAAATTTATCAAAAATGCCAATAACTCA
ACACAGATTTGCACCCTAACCAAATATTACAAACACCACTAGCTAACATAACACGCCCATACACAGA
CCACAGAATGAATTACCTACGCAGGGGGTAATGTACATAACATTAATGTAATAANGACATAATATGTA
TATAGTACATTAAATTATATGCCCCATGCATATAAGCAGGTNCATGACCTCTATGGGGGTNCATAATA
CATATAATTATNGATNGTNCATAGTACATTTATGTCAAATTCATTCTGGATAGAATATCTATAATATATT
CCTTACCATTANATCACGANCTTATTTACCTTTGCCGGGGGAAACCANCACCCGGNTAGGCAGGGATCC
TTNTTNTCGNTCCGGGCCCATAAACCGGGGGGGTGGTTATNTAATGGAATTTTACCAGGCATNTNGGT
TNTTTNTTCAGGGCCATCTNATNTAAAACNGGNCCATTNTTTCCTTNTTAAATAAAACATTTCGANGGA
CTAATGGGTTAATCAGCCCTGCCTTCACACATAACTGGGGCGGGCATAACATTTGGGANTTTTTTTAAT
TTNGGGGGGANGCTTGGACTTCAGNNTTGGGCCGGNAAAAGGGCCCTGGACCCNGGAACATTTTTTTG
GGNCCTGGGNCTTAAACTGGNNNTTTTGGGNCCCCCCCCTTTATGGGNTAGCCCTGGGNCNTTTNCNG
NCNATNGGGNCCCNGGGACTTNAANTTTTTTTTTTTTTTNCCCCCCTTTTTTNAAAAATTTNCCCCCCTT
TAANATTTTTNCCCCC
TTGANCACAGCTGGAGCTCCCCGCGGTGGCGGCCGCTCTAGAACTAGTGGATCCCCCGGGCTGCAGGA
ATNNNNNACNANGCNCTNCCCNGANNCAANTGCAGAGNCNNACCATTGGTCAATGNATNCNAATGAA
NGCTGTGNATCANCTCATGTNCAANTCCAAGAGCACCACATTGNTNATNAANCGCGCTGGTCTATCTA
AACCAGACTGGNCCNATNAAAANCGGACTTAACATGCCAACCANTTTTTTAAGCNGCNTANTTTNANTT
GAANACNTCNCTACTGTAATGCTCGNCCAATGANGNTNTTACCTNGGTATGCGTCTGANACCCTCGNA
TCTTNTACTTNGGGGNAAANCNNATATCNTNTNNAAAGNGGGGGGNCNCCTANCCCTTCATCCCANTT
GGGGCGAAGAANAAAGNCAGANCCTGANGATGAGCTGCTGTTTTGATTTTTTTNATAGTTCCCCANAA
NGGAGCCTACTGGGAAAGTGGAAATCNAAAAAGAAAAGGGCCAAACCCCCTGTCTTGCAAAAGTCTC
GGACATCCAGCGTCAATTNTTGTNAACCGCAGTGTAACCNCCNTTCAAAGCCGTTNAAANAAAATTCC
TTTTTGGGAGCCCAAAAGCCCTTGCCTTTTTNTTGATTCANNATTCTGGGCCNTNGAAACCCNCGAAAN
AATTTTTNAAAAGGGAAAAATNTNTTATTCAAAANGCTTNTCTGGGACCTGGNCCCCCAANCCNATGG
CCTTAACCGATGAATGGGCNGATTCACAATTTGAAACTTNGNTTAACCTTTTGGCCTGGGAGAAACCGA
TTTNTTGGNGNAAATTGAAAGTTTTANAAATTTAAAATTCAAGNAAAATGGGCCCNTTTTTCCTTTCAA
AGGGTCCCAAGGGAAACTTCAAGAAANAAATTTANCCAAAGCCCGGGNGCCTGGAATTTTGGNAAAA
TTGGGANTTNTTTGNCCCTTTTCAAANAATTNGGATCNAAANAANTCTTAGAAACNNT
NAAAAGCTGGAGCTCCACCGCGGTGGCGGCCGCTCTAGAACTAGTGGATCCCCCGGGCTGCAGGAATT
CGGCACGAGGGGAGAATTCAAGTGTGATCCTCATGAGGCCACATGCTATGACGATGGGAAGACTTAC
CACGTGGGAGAACAGTGGCAGAAGGAATATCTTGGTGCCATTTGCTCCTGCACATGCTTTGGAGGCCA
GCGGGGCTGGCGCTGTGACAAGTGCCGCAGACCTGGGGCTGAACCCGGTAACGAAGGCTCCACTGCC
CACTCCTACAACCAGTATTGCCAGAGATACCATCAGAGAACAAACACTAATGTCAACTGCCCAATTGA
GTGCTTCATGCCTTTGGATGTACAGGCTGACAGAGAAGATTCCCGAGAGTAATATTTCCAACCCAGAG
AAACAAGCGTGGATCTCTGCCAAGGTCCATCCAAACTGGAGTGGTGTTCGCAGACCCCATGTTAGTGT
GTTCCTTTCTTAAGCCCTTTGCTCTGGAGTAACTTCTNCAGCTTCAGCTCAACTCACAGCTTCTCCAAGC
ATCACCCTGGGAGTGTTTTCAGACTTTTCTCATACATGGGAGCAGTAGGTTATCTATTCTTCAAAATAT
TCCATGCCAATCAGTATTTTAAAATGAAGTGATTCTNACTTGNGAATTTGGATTGGGAATCCATAGGG
AAACACATGCCCCCAACCAAGATACCNAAATGTCNTTTGAAATGATATTANCCAAAAATTTTAAGTANG
GAAAGGTANCCTGAAGACCTTTTGGNTTCCACTTAAACTGGACTGGGCCCCGCNAATANTGNAAGGAA
ACAGCATGNGCCCTTTGGANCTGGGGGATTCAAAAACAAGNCCCNGGTACTTCCNTTTTTTCCNAAAG
GAATTNTAAGNAATTGGCCTAAAAAAAAATTNTTTTTTNTTACCCGGNAATTTNAAANAANTTTTTTCC
CCAAANTTTT
ANCANNGCTGGAGCTCCACCGCGGTGGCGGCCGCTCTAGAACTAGTGGATCGCCCGGGCTGCAGGNA
NGATCAANCNANTCCTGGCCAAGAAACAAAAGCAGAATCGTCCCATTCNNGAATGGATTCGAATGAA
AACTGGCAATAAAATCAGGTACAACTCCAAGAGAAGACATTGGAGAAGAACCAAGCTGGGTCTATAA
GAAGCAAGCTGGGTCTATGAGAAGTGGTCTTAACATGTAGACCACTTTTTTAAGCAGCCAGATCACAA
TGAAAACATCACTACTGTAATGCTTGGCCCATGATGTTATTTCCTCACTATCAGTCTGAGACCCAGCAA
TAAATATAAAACGTTGCAAAAAAAAAAAAAAAAACNTGGGGGGGGNCCCGGNCCCNNNTTCNCCNN
NNTGGGGNTCNTNNNNNACACANAGNACNCCATGTCCTNNTCCNNGTNTTNGNTNNTGCNTCNANTNT
TGNTCGNTTTAGNCCTTTCTNCTTNNTTTTTANTTNCCANTNNTGGTGTNANNNGCTTNTNGACCTTNAT
NNGNTNTNNNATCCGCCNAATCAACACNAGNTCCNACNNTTTCGGGNANTNNTTNNCTATCCCNANNT
CGAACTTAACAGTCAAGNTTTTTNGNTCTTNANCCANATANTTTCNNNTATTNNNTTTNNGGCNNTNT
GGGTNTNTNCTNNTNNACNNNACATTNGGNCTTTNTGANNNCTCNTNTCNTNGGGATTTCCTTTTTGNN
CTACNTGATAANNCAAATNCTATNTNNNGCATTGNTTTCTNACANCNGTGTNNNCTCNCTCATGANATTT
TTNANNNNNTANTNNATTNNNTTNNTNCTTTNNTNTNCGTCANAATNNCGTNTCNANCNCNNNGNANA
TNCTTTNTCCTTNCTAGTNCGCCCTNNTTTTGTACCTCCCTCTGGNCCGGCNTNAACNACCNTTGNAANA
GTTCNTGTNNNTACNTTTCTCTTCTACGTCANCNTTANCTAGTTCNCTTTTNGTTTNTCTTANTCTACNN
TNCNTCCC
GNTGGGNCGNANATNNNCNCANATNNNTTTCCCNCCCCNNNNCCCTTTTCCCTCCTTCNCCCAAACTC
CCCCCNCCTATGGNGNNNGAAAGTNATATTTTTCNCNTNCTTGAGAAANACTGGAGCTCCACGCGGTGG
CGGCCGCTCTANAACTAGTNTCATCCCCCGNACTGCACGATTTNCGACATCCTTNNNNTTTTTTCTTAA
TNCCTCAACNTCCGCNTTAAAANATNGGGGCNTCCCNANNGGGNTANTTAAAAANNTACGTCACNNA
NCTTACTATTTTTTCAANNAGNNTGAAATTCCNNTTNTNNNTANNTNTTNTTATNNCTANTNGNTCTTCAN
NNTAATNNAANNTNNATCACCATCNTNTNANTTTCANTAAGAACTANNTNTAGATCATATTNTNCAAN
NTGNNTTNTATNTCGANNANTTATNATNTCNCAAANNNTNTGNNNTTANTGATNTTCNATTTTTTNACT
ATCCAAAANTNNNCGCNTNAAANATNNCNCAATAGTAGNCNNTNGGNCCANCNGTNATNATTTTCTA
TCANCANTTACTCTANNNAGTCTAATTNTTANCTTNTGGTNANNTNATTTAGNTATNGNNATCAANCN
NATTTAAANTTNTNGTACTANTCATATNNNGTGNANTTTNTTCNTNANTCGATNNATTATAGNTCNNCG
CANAGGNNATTTTTNTNGAACANNCATTATATANNNNAANGNATAANTANNTNNGNNAGAAGNNTTT
NNNNNGCTCTTNTTCTNNGCAANTGNNTANNTNNTNAACCTAACTCNACNTCAGNTANCTTTTCNCNN
NNCCNTCANNAGTNNCGNNGTNAANCANNCANCNGGCNTNATTATNCNATATGTNTNNNATCANCGA
TGCGCNNNNAGNNTACTGTNCANCGTNNTGTATNNNCNNTNGNNNTCNTANTNNGNANCTCAGCATA
TTAGCTNANGTTTNTCTGNAANCGCCTNGCNTTANNTNTNTACNATCNGNATATAGATTTNTGNCTANT
NNTTNTACGCCTNAGCNNTNTNNGNNTGCNCGCCNAATNCAGANTNTCGNATNTGCGNACNTNCNNN
GGANNATATACTTANNTGCACNGATATCTNATATATCTNCTGATNNGTCANNNANTNCGATCATAANT
NTCGNATCATTNNNNTANNTANNCTGATATGCNGCNNTNNTGCGCACACNNTGAGACGNNANNTAAGA
NNNNGTGNNATANTATNACATTTTNACAANGNGAANNGACACNCNACNNNTNTNGNTNANNTAATGA
TGATACGTNTNANAGATAGANCGANATATNNCAGNGTCTGNANGTGCNATANTNCNCGANTCNATCA
CTTACTCATTTANGCTTNTNNTGNTCNCGAGATNANNCNNTNATANATNTAGNTGTCTNAAGANAANT
TCNTACNCNNGTCANATATNTCTANTGANNANNTGTTACGATAGTNTACGTACGANCNNAGTNTCNCT
CNNANAGNCANNACATATACTATNACGACNANTCTAGACNANATACGNCANAGNNANGTGANTAGN
ANTNNCNTANTCTNTNNTCNCTACATTAATANNNATAGTGAGACGNGTNNCTTNATANNCTAATACNN
TNNGAGTNNTNNAGATATATGCTNAATCTCTCGCC
NNCAANANCTGGAGCTCCACCGCGGTGGCGGCCGCTCTAGAACTAGTGGATCCCCCGGGCTGCAGGC
AGCTGCAGCGCCTCTCGTCTTGTAGGCTCTCCTAGCTATCGCCTTTTCGCTTCCGGAAACATGGCCTCC
GGTGTGGCTGTCTCTGATGGGGTCATCAAAGTGTTCAACGACATGAAAGTGCGTAAGTCGTCGACACC
AGAGGAAGTGAAGAAGCGCAAGAAGGCGGTGCTCTTCTGCCTGAGTGAGGACAAGAAGAATATCATC
CTGGAGGAGGGCAAGGAGATCCTGGTGGGTGACGTGGGCCAGACGGTAGACGACCCCTATGCCACCT
TTGTCAAGATGCTGCCAGACAAGGACTGCCGCTACGCCCTCTATGATGCAACCTACGAGACCAAGGAG
AGCAAGAAGGAGGACCTGGTGTTCATCTTCTGGGCCCCTGAGTGTGCACCCCTTAAGAGCAAAATGAT
CTATGCCAGCTCCAAGGACGCCATGAAGAAGAAGCTGACGGGGATCAAGCATGAATTACAAGCAAAC
TGCTACGANGAGGTCAAAGACCGCTTGCACCCTTGCAGAAAAGCTGGGGGGCAGCGCTTGTCATCTCC
TGGANGGCAAGCCTTTGTGAGCCCCTTCACCCCTGNCTGGACATCTGGCAGCCCCAGACCTGNCCACG
GGGGTTGCANGCTTGCCCCTTCTGCCANACCGGAGGGCTGGGGGAATCCCNCAGGGGGANGGCANTG
CTTTNCCCANTTGCCAAAGCCCCCCCGACCCCTGGACNTTNTTTTCCTTNACCCTTGANGTTTTGGCN
TTCCNAACCGNTTTNGAACTTNNANCCNTTTGGGGTGGAAAAAAAACAAATTNCCCCCCCAGGAANCC
CTNTTTGGGGGGGGCCTTTTTTTTTTTAAAAAAACCCCAANNCCCTGCCGGNNCTNCCCTTTNCCAGNN
NGGNA
GCCCTATNNGGAGCNGCATNACAGTGGCGGCCGCTCTAGAACTAGTGGATCCCCCGGGCTGGAGGGTT
TCATGAAGATGGAGAATGCTGNGTNTATGATGAACCATTACTGAAGNGNCTNGGNGCTGCCAAGCATT
AGGTNGGGANNCACAAAGTTTGCTCCTGGTTCCAGGGCACGATGCATAATNAAGCAACAATCTTCTT
TGGGAGAACCTATTCACTCCCTTCTTATTGCAGCCGGNCNCTATCTNCANGGNATACTTGNACTTTNTG
ATGCANCATGANCAATTAANGCGAGCAAAGCNCCCCTTTTTNCTGNTGCGTGGCAAGTNACAACNNTN
GANTTTGGCCTTGATAAAAANTTCAGGTTTTGAAAACAAGATGTCTTGCTCCTTTTCCAAATCCCACGG
NATAAATCCCAGTNTTAAACNCTGCATTCTANTACTGNTGNTGAGANATACAATACATGTTTTTCCTAGT
NCATATAATCNTTGGGTTNGTATATATACATATNNTATAATTNTATGCTGATGCCCGGATTTTTCATAN
NATACCTGCATTTTACTGTNAAACATCAAGNCCCCTCTGAGTTGTTTTTANNANANATTTNCNGCNNTT
GCGGTTAAAAATTCNCCTCGGNTNTGAAAANTTCCATGGGTCCNCTCCTANCNANNCAANNTCNTCAC
TTTGGNAATTGAAAGTANTATTNNCTCAAACNNGNATCTGTTACCTCTTGGNCCTGGGGGTTTTAANNT
NACACCTCNAANGGCTTTCCTNNTAGGNTTNCNNANNNGCTANATTCNNTTNTCAANCTNTGCTTCNT
TTCAAGNANGCCNGGNCTNNTTNTTTNCNGAANTACAANTTTGGGACCCGNATANTCACNTCANNGNN
CTTNTTGATNNNNNNNAANGGGCTGNNGCTTTTTGTNTNGTGNNNNNCTNNTTNACNATNAACANANA
GGNNGGCTNCNTNTACANTNNCTNGNNCTNCTTTCNNTATAANCCCTTNNGTNNNCCANGNCNNANA
ANACCG
GNNGGGCCGGNNNATNNNTTATNTTTNTNNTATNTTTTTTNNNANNNTTTTTTTGNTACACCTTANTNN
GGGGNTCNNCNGNGNNTTCAATGGNGGGNGCGGNGGGCCNNCAGNNTTNNGCCCNCGGGAGTCGCAT
NACANGGNGGCCGCTCTNCANTATGNNTTCCTCCGNNCTGCANCATCNGCNCNNNCNCCTCNTTCNTT
NCNTCNCCNGCNTCNNCTNNGTCNCAAGGGGGANTTANTNTANNNAGTNTCNNNNTTGNCAACT
TATNTNTNTTATCCNTNATATNGNAGGCTNNANTCNGNATCTNGAGANNCGGGCTNTCCNNNCNTGC
TTTAANTTNTTTCNANNGNNGNCANGANNNCNCANTCTATNCTNNANNAATNTNNNATGTCNCNNNA
NNTTNTCGCACGANTCNTCGTTNNTNCACAGNNTNTGTTNTTCTNGNNCNCCGTNNNCTTTCNNNNC
TNNNATNCCTCAGTTTTNTTNNNNCNCCNNACNATTNTCTCNATCNCANTNCNTNTACNTNTANCATCG
TTNNNANANATCNNTNCNTTNNNNTNTTTNCTACNTCTNCTNANANTTATTTCCNCNTCTANTCNNNTA
ANNNTTCGNNTTNACCTNTTTAGANNNNANNGGANNTTTCCCNNCTNCNCNNGATCNCGTTNCCCNNN
TNCTCNNGTCNANCCNACTCATTGCCTCTATNNTCNNTANTNNTATNTCTTTNGANCGCCNTNNGNATA
CCATTNATTATAATACGNANATNNNTCNCNCACGNGNNCCCANTNTCTCTNNCNNNNGGNTTNTGNTN
ANTNTCCCAAATCNCCNNCNGACCCNTNNATGGNCCNCCNNNTANNNCCANNAGNNNNCGATTAGNA
CANNNGNTNTNGGACGNTCNNATCNATNNTNTNCTGNNGCNTAGNCNATTTTCNANNTGNTGCTNGCG
NTANTGCNCNTNTCGNNNTAATCGNTGTNTGTTNANNNTGNNCTCTANTNGNCNGGCNNTNGGTNNNC
TCTANTGAGTCCACGNTNNNNTCTNACTCTGTTGNNACGNGATTGCTCNANCTNACNGCTCNTNTA
TNCNCNCNTGTNNNGCNNNAGTNATCNTCCTATTGTNTGTTACTCCTTNTTAGNCTACANNGTTTNGCT
NTNNNTGCTTGACCNCTNTAGACCGNGCTTCCNNTNATCCTCATGNGATGTNTNNCNTGNTANNCNCC
TCNTCCGCTNTTNNCNTNTCNANTGATANGAGTCTNNNNTNATNCGCNNTGTNNANGTANTANNATAC
NCNNANCTCNATGNCACTCCAGTGATGNANGTNNTATANNTAGTCTNNGTCATCGTANCANTCNNNAT
GGTACNNANTCTCTNGNGTNGNCGCGTNGTATGTGGNNGCNNTNTTCNATGACGNTNTANAGCAGTA
NNGNTACACNTNTATAGTCCGTNTNACNAATCTCTNGNTTNANCNTNTATTCNCNNCNGCTCNCTCCC
GN
GNNGGGCCGGNNNATNNNTTATNTTTNTNNTATNTTTTTTNNNANNNTTTTTTTGNTACACCTTANTNN
GGGGNTCNNCNGNGNNTTCAATGGNGGGNGCGGNGGGCCNNCAGNNTTNNGCCCNCGGGAGTCGCAT
NACANGGNGGCCGCTCTNCANTATGNNTTCCTCCGNNCTGCANCATCNGCNCNNNCNCCTCNTTCNTT
NCNTCNCCNGCNTCNNCTNNGTCNCAAGGGGGANTTANTNTANNNAANAGTNTCNNNNTTGNCTAACT
TATNTNNTNTTATCCNTNATATNGNAGGCTNNANTCNGNATCTNGAGANNCGGGCTNTCCNNNCNTGC
TTTAANTTNTTTCNANNGNNGNCANGANNNCNCANTCTATNCTNNANNAATNTNNNATGTCNCNNNA
NNTTNTCGCACGANTCNTCGTTNNTNCACAGNNTNTGTTNTTCTNGNNCNCCGTNNNCTCTTTCNNNNC
TNNNATNCCTCAGTTTTNTTNNNNCNCCNNACNATTNTCTCNATCNCANTNCNTNTACNTNTANCATCG
TTNNNANANATCNNTNCNTTNNNNTNTTTNCTACNTCTNCTNANANTTATTTCCNCNTCTANTCNNNTA
ANNNTTCGNNTTNACCTNTTTAGANNNNANNGGANNTTTCCCNNCTNCNCNNGATCNCGTTNCCCNNN
TNCTCNNGTCNANCCNACTCATTGCCTCTATNTCNNTANTNNTATNTCTTTGANCGCCNTNNGNATA
CCATTNATTATAATACGNANATNNNTCNCNCACGNGNNCCCANTNTCTCTNNCNNNNGGNTTNTGNTN
ANTNTCCCAAATCNCCNNCNGACCCNTNNATGGNCCNCCNNNTANNNCCANNAGNNNNCGATTAGNA
CANNNGNTNTNGGACGNTCNNATCNATNNTNTNCTGNNGCNTAGNCNATTTTCNANNTGNTGCTNGCG
NTANTGCNCNTNTCGNNNTAATCGNTGTNTGTTNANNNTGNNCTCTANTNGNCNGGCNNTNGGTNNNC
TCTANTGAGTCCACGNTNNNNTCTNACTCTGTTGNNACGNGATNTTGCTCNANCTNACNGCTCNTNTA
TNCNCNCNTGTNNNGCNNNAGTNATCNTCCTATTGTNTGTTACTCCTTNTTAGNCTACANNGTTTNGCT
NTNNNTGCTTGACCNCTNTAGACCGNGCTTCCNNTNATCCTCATGNGATGTNTNNCNTGNTANNCNCC
TCNTCCGCTNTTNNCNTNTCNANTGATANGAGTCTNNNNTNATNCGCNNTGTNNANGTANTANNATAC
NCNNANCTCNATGNCACTCCAGTGATGNANGTNNTATANNTAGTCTNNGTCATCGTANCANTCNNNAT
GGTACNNANTCTCTNGNGTNGNCGCGTNGTATGTGGNNGCNNTNTTCNATGACGNTNTANAGCAGTA
NNGNTACACNTNTATAGTCCGTNTNACNAATCTCTNGNTTNANCNTNTATTCNCNNCNGCTCNCTCCC
GN
TTNNNAAAGCTGGAGCTCCACCGCGGTGGCGGCCGCTCTAGAACTAGTGGATCCCCCGGGCTGCAGGA
ATTCGGCACGAGGCAGCAACCGTCGCGATGTTGATGCCTAAGAAGCCGGATTGCCATTTATGAACT
CCTTTTTAAGGAGGGGGTGATGGTGGCCAAGAAGGATGTCCACATGCCCAAGCACCCGGAGCTAGCA
GACAAGAATGTGCCCAATCTTCACGTCATGAAAGCCATGCAGTCTCTCAAGTCACGAGGCTACGTGAA
GGAACAGTTTGCCTGGAGACATTTCTACTGGTACCTCACCAACGAGGGCATCCAGTATCTCCGTGATT
ACCTCCACCTGCCCCCTGAGATCGTGCCCGCCACCCTGCGCCGNAGCCGTCCTGAGACTGGNCCGGCC
AAGGCCCAAAGGTCTGGAAGGAAAGCGACTTGCAANACTCATTCGAAGGGGAAGCCGACAGAAAAC
ACCTTACAGACGAAGCGCCGTGCCCCCTGGTGCCGACAAGAAGGCCGANGNCGGGGCTTGGTCAACA
ACTTGAATTCAAGTTTAAAGGCGGATTTGGTCGTGGACGTGGTCAACCNNCTTANTGAAATTGAAANG
GAATGGTTTTGTGTTGAATAAACCTGTTACCCGAAGAAAAAAAAAAAAAAAANGGGGGGNCCCGGN
NCCCAAATTCNCCCTTTTAGGGGNGNNNNNTNAANAANNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNN
TTGANACAGCTGGAGCTCCCCGCGGTGGCGGCCGCTCTAGAACTAGTGGATCCCCCGGGCTGCAGGCN
NNATNAAGCNNNTCCTCCCAAGAANCAAAAGCAGAATCGTCCCATTNCTCANTGGATTCGAATGAAA
ACTGGCAATAAAATCAGGTACAACTCCAAGAGAAGACATTGGAGAAGAACCAAGCTGGGTCTATAAG
AAGCAAGCTGGGTCTATGAGAAGTGGGTCTTAACATGTAGACCACTTTTTTAAGCAGNCAGATCACAA
TGAAAACATCACTACTGTAATGCTTGGCCCATGATGTTATTTCCTCACTATCAGTCTGAGACCCAGCAA
TAAATATAAAACGTTGCAAAAAAAAAAAAAAAAANNNTGGGGGGGGCCNNGGCCCCNNTTNNCCCCTT
NTNGGGGNGGNNNTNANACTNGGGGAACCTTNACTAACCANATCCANGCCGCATTCANGGAGCCAAN
GTTTTTGGTGGGCACCGATNCCAGGGCTGACCACCAACCCCTTACGGGNAGCCTNTTACNTGAACCTG
CAACCATTGNCCTTNTGCAANACGGGCTNTNCTNTGNGCTACGTGGANATTGNCATTCCNTGCAACAA
CAANGGGAGNGCNCTNANTGGGTCTNGAGTGGNGGATGCNTCCCCGGGAAGTCCTGNGCATGCTTGG
GACCCNTTTTCCGANAAGCNCCNCGNGGGGAGGTCNTGCCCGGACCTTTTACTTTTTACANAAAATNC
NTNNGGGNGAATTNGAAAAGGGAANNAANCNNNCNNNNNNTTTTANAAAGNNNTNTGANNCCNNGG
NNGGAGTTTNNNGGGGNGAANNGGGNTTNTTTCCACNTTCAAANTTTNCGGGTNNTTNNNNCCTTAG
GGGGGNNAAAATTGNNTTTNAANGGGNTTNNAANGNNGCCTTTTCNNGTNCCCTTTTANNNNNTNTCC
CCCCTTNNGNAACTTNGANGGCCTCC
TTTGAANACAATCTGGAGCTCCACCGCGGTGGCGGCCGCTCTAGAACTAGTGGATCCCCCGGGCTGCA
GGANTTNNGCACNAGGNNTTTANCTAAAAANCNNNNTNNTCNGGGCANNAGGGGGGGNNNCATNCT
GACNATNGCNCNCGCNCNACNNACCCGGGNANTNNGGNCNANCNNTNNCNGNCATGAGGCTNNCAC
ACCNAACNNANATCACANNTNCCCCATNATTGNNGACNCCNGCNTNCNGNCATGANAANNNGCTGC
CNTCCCAATGACNNTNNTTACNTCACNATATACNTGGTATNTNCGANCANCNCTTNANTTTTTAANTCC
ACTTNNNCCAAGGGNANACTACCANNCTTNAAATCTNTTANCCNNGGGNAGGGCNAAATNCCCTGNC
AGNCNANNANTANNAACNGGANTTTNCNANGGNNCGANCNNGGACGNAACCTTTTNGGGACCTTGCC
NNANGGCCANNTNTNGGCCCCATGANTGCCCTNNNTNNNNNAGGNNNACCCTCTTNNAANTNGNNGT
NNNGNNNATNNTCNCNNTNCNACTACNNCACANAANTTTGGGCCNNNCCCCCNTCTNGNNCAAACNA
NGATNTTCTNTCTNCCTTGAAANGGANNAAAAAANAANTTTCTTATTTAACCACCATTAAAAANNNNT
CGNGGTNGGNTCANNNCANCTNCCCCTNCTCNAAAANACCNTTTANTCCCTTNNGNNTGNANNANA
NGNNTNNCCTNNNCCCCCTTTNCNCTTTCNNCANNNNTNACTNNATNTCCCCNNCGNTCTCNCCTNNN
TCNTNNNTTNTTCCCCACCNGNTTNCCGAAAAACCTNTTNCCCAANANCTANNGTTTCNCCANNNTCC
NNTNCACTCCTATTANNTNNATNNTNTNNGTTNNCTTNATNTCTCCTCANNANCNATAGCTNNNTNAC
TNCCNTTTTNTTTNNCTCCANTATNCCGCCNTCTACTNCTTCCTNACTTCNATCAANAATTACNNTNNN
NTNCTCCGTCGTTNCTGTTTANTCNTACCTNTACTACNNCANNNGTTAATNTTGTNCCCTGNCTCNNN
TCTTCTNCTCATANNCN
AAAAGCTGGAGCTCCACCGCGGTGGCGGCCGCTCTAGAACTAGTGGATCCCCCGGGCTGCAGGAATTC
GGCACGAGGCAGCAACCGTCGCGATGTGATGCCTAAGAAGAACCGGATTGCCATTTATGAACTCCTT
TTTAAGGAGGGGGTGATGGTGGCCAAGAAGGATGTCCACATGCCCAAGCACCCGGAGCTAGCAGACA
AGAATGTGCCCAATCTTCACGTCATGAAAGCCATGCAGTCTCTCAAGTCACGAGGCTACGTGAAGGAA
CAGTTTGCCTGGAGACATTTCTACTGGTACCTCACCAACGAGGGCATCCAGTATCTCCGTGATTACCTC
CACCTGCCCCCTGAGATCGTGCCCGCCACCCTGCGCCGCAGCCGTCCTGAGACTGGCCGGCCAAGGCC
CAAAGGTCTGGAGGGAGAGCGACCTGCAAGACTCACTCGAGGGGAAGCCGACAGAGACACCTACAGA
CGAAGCGCCGTGCCCCCTGGTGCCGACAAGAAGGCCGAGGCCGGGGCTGGGTCAGCAACTGAATTCC
AGTTTAAAGGCGGATTTGGTCGTGGACGTGGTCAGCCACCTTAGTGAAGTGGAAAGGGATTGTTTTTG
TGTTGAATAAACCTGTAAACAGAAGAAAAAAAAAAAAAAAAAAGGGGGGGCCCGGNNCCAAAATTCN
CCNTAAAGGGGGNNNNTAAAAANNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
AAAACTGGACTCCACCGCGGTGGCGGCCGCTCTAGAACTAGTGGATCCCCCGGGCTGCAGGCTCCTGG
TGAGCAAGGTCCTTCCGGAGCCTCTGGTCCTGCTGGTCCCCGCGGTCCCCCTGGCTCTGCTGGTTCTCC
CGGCAAAGATGGACTCAATGGTCTCCCAGGCCCCATCGGTCCCCCTGGGCCTCGAGGTCGCACTGGTG
ATGCTGGTCCTGCTGGTCCTCCCGGCCCTCCTGGACCCCCTGGTCCCCCAGGTCCTCCCAGCGGCGGCT
ACGACTTGAGCTTCCTGCCCCAGCCACCTCAAGAGAAGGCTCACGATGGTGGCCGCTACTACCGGGCT
GATGATGCCAATGTGGTCCGTGACCGTGACCTCGAGGTGGACACCACCCTCAAGAGCCTGAGCCAGCA
GATCGAGAACATCCGGAGCCCTGAAGGCAGCCGCAAGAACCCCGCCCGCACCTGCCGTGACCTCAAG
ATGTGCCACTCTGACTGGAAGAGCGGAGAATACTGGATTGACCCCAACCAAGGCTGCAACCTGGATGC
CATTAAGGTCTTCTGCAACATGGAAACCGGTGAGACCTGTGTATACCCCACTCAGCCCAGCGTGGCCC
AGAAGAACTGGTATATCAGCAAGAACCCCAAGGAAAAGAGGCACGTCTGGTACGGCGAAGAGCATGA
CCGGCGGATTCCAGTTCGAGTATGGCGGGCCAGGGGTTCCGATCCTTGCCGATGTGGCCATNCAGCTG
AATTTCTTGCGCCTGATGTCGACGGANGCCTTCCAANAAATCACCTACCACTGGAAAGAACAGCGNGG
NCTAATGGACCAAANCANACTGGCAACCTTCAAAAAGGCCTTGTTCTTCANGGCTTCCAACGAAAATC
GAAAATCCGGGCCGANGGGAAAAAGCCGGTTTACCTTACAGGGTACCCTACCANGGGTGGCCAAANC
AACCCGGAACCCTGGGGGCCAAAAAGNGATTGGAATACCNA
TTTGAGNCCTTANANGNGNNGTCGGTATTAACAAGGGGANGGGCGCGNTATAGNAACTAGGTGGTAT
CCCCCGGGCNTNGCAGGNATNCANATTCNNTGAGGGNNTTNNCTTGAGCCCTGTAGGGGAGNNCNCG
TNNCTNGGGGGCNNNTCNNATGANCCTAGTACNNANACNNNTCTNTTNAGNNNATNTTTTNNNGTGN
CNNTANANNNGANNNGATTGCNTNGAGCGTTNNNNNGGNTNNCTTNNATNCGGGNATNNGNAGCNAT
TGTNTGGTTAGTTGNNTANAGGNNNTATNNTGANNATCCNNTGNNNNNGNGGAANGCNNAGGGNGNN
NTNCGANNGTNCTNTNTNNNTGNACGATNTGACNNNNTTNNTCGATNNNANAATNNTNTCNNNCCAANN
TTNNGCTANATAGNNTNGTGNNNNGCNANTAAACNNNCNANNNTANNNANNGATTCCANATNNNCCT
NAANNCGNTTNANCNNNGTNNNNNAATNANCNTGGNNNCNGNTATNNNTCNAATNNNNNANNTCG
NGCAGCNTATGNACAATANTGGNTANNTNNANTNNNATTAGNNCNACTGATACNGNAANTNTNTNNC
CTNNTNCNATTTNNGTTTAGNNNNGNNGTNNNNAGNGGGTNNANTTNTNNNNNGGNGTNTTCGTGTAN
TNNAANGTNNNNAGGGNNGGGNNNTGNNANNTNNGAANGNGGANANNTATGATGCNCNNNAGTACT
TNTGGAACTTCATTNGNANGGGGNTNNGTNTCTGNANGNGNCNNNNGNTGCATGCNTANTNTNNNA
NCCNNTTATNGCNCTCTGTNTGNGGNGNTNNNTGGANNTTNGGNNNGTTTANNNNGNNTNGNNGTAG
ACNTGTGTCGNNNNAGGGTTTNTNTGNGCTNTCNGGATGTTGNNTTTGANCAGTNNGTANATNANNGG
NTNANTANGNATNANATNNNNTAGGACGGTNATGGGAACCNNGGTANATNNTTTTGTNNNGTNNNNA
GTANNNGGTGNAGNNANTANATCTANAANCGAGGAACCNACNTTNNANNNGNNNNNTTCGNNNTGN
AGTANTAGNCGGCGCNGNTAAGTTANCTTGCNACGATNCGTTGTGTCNCNCNCGANATANGCGNTT
NNACGTTNCTGNTCNNNCACGTNGCTCNNNTNAGNGTTCGNTNNCNTATGNAGGANTNTGNACGTNTN
CGAGNGAACNNACATCGTCGNATGGCNTNNAATGNGNGCGTATTNGNCGTNGNNGACNANTGACGCA
NCNATANNNCTNTNNGTACGTANGTAAGTCNNANNNTNCTNNNTGCG
NCAAANNCTGGAGCTCCACCGCGGTGGCGGCCGCTCTAGAACTAGTGGATCCCCCGGGCTGCAGGCT
GAAGAAGAAGTACCCAATGGCCAAGTACCTGTGTGTGTTGCTAATTGTGGCTGGAGTGGCCCTTTTCA
TGTACAAACCCAAAAAAGTAGTCGGGATAGAAGAACACACAATTGGCTATGGAGAGCTGCTCCTGCT
CTTGTCTCTAACCCTCGATGGACTGACCGGTGTTTCCCAGGACCACATGCGAGCTCACTACCAAACAG
GCTCCAACCACATGATGCTGAACATCAACCTTTTGGTCGACACTGNTGNTGGGAGCTGGAATCCTGNTC
ACCGGGGAGCTCTGGGAGTTCTTGAGCTTTGCCGAAAGGNACCCTACCATCGTCTATAACATCCTTCTC
TTTGGCCTGACTAGTGCCCTGGGCCAGAGCTTCATCTTCATGACAGTTGTGTATTTCGGTCCCCTGACC
TGCTCCATCATCACCACAACTCGAAAGTTCTTCACCATTTTGGCCTCTGTGATCCTCCTCTTCGCCAACCCC
ATCAGCCCCATGCAGTGGGTGGGCACCGTGCTGGTGTTCTTGGGTCTCGGTCTTGATGCCAAGTTTGGG
AAGGGAGCCCAAGAAGACATCCCACTANGAAGAGAGAAGACTAGCTCNACTCCAAGAATATTTAAAN
TTATTTTCTCCAACAGTNGACATCTCTTGGGAAAACGGACACAATTAGAGATAAAGGGACTATTGTC
ANTCTGGGAAAAAANANNNNNNACNNNNNNNTNCCNNNGGGGGGGGGCCCNNGGCCCCNAATNTTCC
CCCCTTTTANGGGGGGNGNNNNTNTAAAAANNNNGGNNNNNNNNNGGGNNNNNNGNTTNNNNNNNNT
NNNTTCNAATNNNGTNNGNNTNNNNNCNNGNNNNNNTGGGGNNNNC
GCCTATAGNGAGNNGNATTACAGTGGCGGCCGCTCTAGAACTAGTGGGATCCCCNGGNCTGNAGGTT
AACGCTGCAGCCTCCGNCGTACCCGCGCCTGANCCNNTGCCAANCGCTTTTGAACAAGAAGANGGNT
GNGCCTNCCANGNATGNTGATCTTGGCAAAACTGNCANGGATGNNTTCACCNAGGGCTATGGATTTGG
NTTAATAAAACTTGATCTGAAAACAAAATCTGAGAATGGACTGGAATTTACGAGCTCAGGTTCAGCCA
ACACCGAGACCACCAAAGTGACGGGCAGCCTGGAAACCAAGTACAGATGGACTGAATATGGTCTGAC
GTTTACACGAGAAATGGAACACTGNCAACACGCTGGGCACGGAGATTACTGTGGAANATNAGCTTGC
ACGTGGCCTGAANCTGACCTTCGATTCATCCTTCTCACCAAACACTGGGAAAAAAAATGCTNNAATCA
AGACAGGGTACAAGCGGGAACATATCAACCTGGGCTGNGATGNGGATTTTGACATAANTGGTTCTTCC
ATTCCGGGGCGCTCTGGNGCTGGGNTATGAANGTTTGCTGGNTGGCTACCANATGAATTTTTGAACTG
CTAAAGTCTCCAAGTGACTCATAAGCAACTTTTNCAGTTTGGCTNACNANAACCGNNGAAGTNCCAGC
TTCNCACTTAANTGTNAAANTGATGGGAACCNCNAGTNTTGGTGGCCTCCCTNTTTTCCAGAAAGGNG
AACNAANAAAGTTTGGANNACCCNCNGTTAATTCTNGCCNTGGACCCNGAANGANAAACAANCAACC
ACTCCTNTTCGGAANTTANCCTNCCCAAGTCNCCANTTTTNNAACNNNNNCGGCCNGGCTTTTNNTCT
NTANGTGGAACCAAACNCCCNCCCTGTTTNGGATTAGGGATATACTCCNNAACCCTTNAANTCCNNGG
TNTTAAAACNNNCNCTTNGTCTGCTTTGNTTGGNC
GCCTATAGNGAGNNGNATTACAGTGGCGGCCGCTCTAGAACTAGTGGGATCCCCNGGNCTGNAGGTT
AACGCTGCAGCCTCCGNCGTACCCGCGCCTGANCCNNTGCCAANCGCTTTTGAACAAGAAGANGGNT
GNGCCTNCCANGNATGNTGATCTTGGCAAAACTGNCANGGATGNNTTCACCNAGGGCTATGGATTTGG
NTTAATAAAACTTGATCTGAAAACAAAATCTGAGAATGGACTGGAATTTACGAGCTCAGGTTCAGCCA
ACACCGAGACCACCAAAGTGACGGGCAGCCTGGAAACCAAGTACAGATGGACTGAATATGGTCTGAC
GTTTAGACGAGAAATGGAACACTGNCAACACGCTGGGCACGGAGATTACTGTGGAANATNAGCTTGC
ACGTGGCCTGAANCTGACCTCGATTCATCCTTCTCACCAAACACTGGGAAAAAAAATGCTNNAATCA
AGACAGGGTACAAGCGGGAACATATCAACCTGGGCTGNGATGNGGATTTTGACATAANTGGTTCTTCC
ATTCCGGGGCGCTCTGGNGCTGGGNTATGAANGTTTGCTGGNTGGCTACCANATGAATTTTTGAACTG
CTAAAGTCTCCAAGTGACTCATAAGCAACTTTNCAGTTGGCTNACNANAACCGNNGAAGTNCCAGC
TTCNCACTTAANTGTNAAANTGATGGGAACCNCNAGTNTTGGTGGCCTCCCTNTTTTCCAGAAAGGNG
AACNAANAAAGTTTGGANNACCCNCNGTTAATTCTNGCCNTGGACCCNGAANGANAAACAANCAACC
ACTCCTNTTCGGAANTTANCCTNCCCAAGTCNCCANTTTTNNAACCNNNNNCGGCCGGCTTTTTCT
NTANGTGGAACCAAACNCCCNCCCTGTTTNGGATTAGGGATATACTCCNNAACCCTTNAANTCCNNGG
TNTTAAAACNNNCNCTTNGTCTGCTTTGNTTGGNC
TTNAAAAAAGCTGGAGCTCCACCGCGGTGGCGGCCGCTCTAGAACTAGTGGATCCCCCGGGCTGCAGG
NCNAGGTCGAGCTNCACTGCGGCAGCAGCAGAGCGGAGTGTACATTCTGATTTGCTACGTTTATATAT
ATCTTGAAGCTAAATGTATATATGAGTAGTTTGCCNTGAGATAACACAGTGTAAACAGTAGACACCCA
GAAATTGTGACTTCTGTGTTCTCTCCATTTGAGTATGTAATTTTTTTGAAATATTTTGTGGACATAAA
TAAAAACAAGCTACACTAATTCATACTNAAAAAAAAAAAAAAAAGGCATNCAGTATNNANGAGAAN
ACCNACANNTAACCCCTGAGATCNAGCCCGCCACCCTGAANCGNNNCTTCCTGNGGGGGGGGGGCCC
CNNCCCAAAGGTNTGNAGGGAGAGCNANTNTNCNNNNNNNNNTNNNNNNNAANCCNNNCNGANANN
NNTACNGACNAANNNCCNNNNNNTTTTGNNGCCNNCNNNNNTNNCCNTTGNCNNNNNTNNNNNNNN
NNCNNNNTTCCANNNNNNANNNNNANNNNNNNNNNNNGNNGNNNANNCCNNTTNNNTNNANNNNNN
NNGGNTTTTTTTNNNNNCCCCCCCCCCCCCCCCCCCCCCCCCCCCCCCCNNNNNNTNNNNNNNNNNNN
NNNNNNNNNNNNNNNNNNNNNTNNNNNNNNNNNNNTTTANAANNNNNNNNNNNNNNNNNNNNNNN
NNNNNNNNNNNNNNNNCCCNNNNNNNNNTTTNNNNNNNNNNNNNNNNNNNNNNNNNNNNNNNNNN
NNNNTTTTNNNNNNNNNNNNNNNNNTNNNNTTNNNNNNNNNNNNNNNNNNNNNNNNNNNNNNNN
NNNNNNNNNNNNNNNNNNTNNNNNNNNNNNNNNNNNNNNNNNNNNNNNANNNNNNNNNNNNNNN
NNNNNNNNNNNNNNNNNNNNNNNNNNNNNNNNNNNNNNNNNNNNNNNNNNNNNNNNNCN
ANCACAGCTGGAGCTCCACCGCGGTGGCGGCCGCTCTAGAACTAGTGGATCCCCCGGGCTGCAGGAG
CTTATTGAATGAAAACTCCAGAGACTGAAAAACCAAACATGAGGGTAGCAGGTGCTGCAAAGTTGGT
GGTCGCTGTGGCAGTATTTTTACTGACATTCTATGTTATTTCTCAAGTATTCGTAAATAAAAATGGATGC
AAGTTTAGGAAATCTATTTGCAAGGTCAGCATTGGATGCGGTTGTNCGTTCTACAAACCTCCCAGAT
ATAAATGTGGAATCTCAAAAGCTTGCCCTGAGAAGCATTTTGNTTTTAAAAGGCAAGTGGAGCAGCC
AATGTGGTGGGACCCAAAATCTGCCTANAGGACAATGTTTTAATGAGTGGTGTTAANAATGTTGG
AAGAGGAATCAATGTTGCCTTGGTAAATGGCAAAACGGGAGAACTAATAGACNCCANATTTTTTGACA
TGTGGGGAGGAAATGTGGCCCCATTTATTGAGTTCTGAAGGCCATACAANATGGAACAATAGTCTTAA
TGGGAACATATGATGATGGANCANCCAAACTCAATGATGAGGCCCGGCGCTGATTGCTGAATTGGGG
AGTNCATCTATTACTCATCTTGGTTTCAAANACAACTGGGTCTTNTGGGGNGGAAAAGGCNTTAANAC
AAAAGCCCTTTTGANCCGCCCNTAAGGAACCNTNGGGGCCCCANTANTTTGAAGGATGGNCCTGTAAN
TTGNGGAAATGGAAAGGATGCCTTTCCCCAAAACCAAAACTGGCCGGGGNAAAAACCCGANGGGAGC
NCCCTTTTCCTTGTTANNGGGNAAAGCCCCTNCCCAAAAAACCTCCNTTTTAAANNTTTTAANAACNTG
CCCCCCCTTTCGGGGGGGGGCCTTTTCCCTTTTTNNTTTTTGNANTAAA
TTTGAANACAATCTGGAGCTCCACCGCGGTGGCGGCCGCTCTAGAACTAGTGGATCCCCCGGGCTGCAG
GAATTCGGCACGAGGANNTAAACTAGAAATCCCTCTAACCTGTGAATGAAGAGGGGTGGACCCTGAG
CAGTGCCTCAATAAAACAAGTAGCCTTACTTGTCAAAATGTAGCTGCATTGAAGTGGCACACTGCTCT
TCTGGTCTCCAGAATTTTTCTGTTCTAAACTTGATTCTCTTAGCCTGTCCCCCACCCGCTTGCTTCCCTA
CCCTCTTCCGCCTCTCACAGGGGTGATTTCTACCTCTTGGGCCCAGACAAGATCTAAAAGAGTTCCCAA
GTGTTCTCATGAGGCGATGTGTGTGCCCTTTGGAGTGTGAGATGCTTCGGTGAGCTGTGGGACCATCAN
GCGTTCTGGGCCCGCGTTATGCAAAGAGCTCCGGGAGCCTTTCCTTTGCTTGACAAATGAGCGTTGCGC
TCCTTGGATGTCTGGCAGAAAGTAGCATACCTGCTTCTCTTCATACATTTTTTAAACTAGTATAAATTGCA
TTTTTGGAAAGCAAAAGGAACTTTAAAATAGTCTGGTTTTAATTCTGCAATAGTTTTTCTTCCCTTCGA
AACTGTTCTGTGAGTCTAANCTGCTGCTCTTTCTTTTTCTCCCTAAAATTCTGAGTATTTCTCCATTGTCC
CCTACGCCGGAAATACTACAGATGTGGTTTNAATCGAACTGGACAGGTTCACCTTATACTGGGNAAGA
AAATTACCTTCCTTAATGGCCCAAGTAAAAATTAACTGTCAAGGTAAAAATATGGTTCATTTGGATTATTA
TTACTGNAGGTTTTATATTCAANGAATTTGGCAAACTTTTTNTTAANCCTGGGACTTCATTGTNTAAA
ATTANTTAAAAGGTTTTNAAAANGNGAACCGGANTNAATCAAATAATTTTGAAACAAAACAATGGGG
CNTGGANTTTTTTTAAAAAAAA
TTGANAACAGCTGGAGCTCCACCGCGGTGGCGGCCGCTCTAGAACTAGTGGATCCCCCGGGCTGCAGG
NANGAACTANGANGNNCNGGNTGCTNNNTTGTCCNGNNCAAGNGNNANGCTGNTGNGGNNNCTACN
AANCTCTGCGGCTNATTGNCTNCTGCAGATGGAGCTGTGCATGGGAGCATCANNCACAGGAGACCTNT
GGTNANNNCNCTGTNCTTNGCCACACCTGNNGNGAGTGNACNGCNTNNTGTTGNCCCTTNCGACCAT
GACNNNACCTNAAANNCNGTGNNGNNGGNTNTNTGANANTATATCCTNTNTNTNTGACTGANACCCA
GCAATNNTTNNGGGGGGTCCCNAANCNNATGGGGAAAGGTNGGGGNGGGGGTNTCCCTTCATTAGGA
AATTGNAGTANGATGTCNTTTTATAGATTGTTTTATTGTATAGTGCTATTTGAATGNTGGAAACGACA
NAATNAAACTGCNCCTTACTCTCNNAAAATAAAAAAAANANANTNTTTNNNCGGGGTTTCNTNTNNN
NTTTTNNNNTNTNNNTNNATTTTTTNNTNAANTNNNNNGNNNNGCNAANCATTTCTTTTNTTTNTTTNT
NTNATNTCTNNNNNNTTNTTTTTNTNCCNNTTCTTCTTNNNTNNNTNNANATNTNNTNCCNTTNTCNTN
NTTNGNNNNNNCTTTCNTTNTNCNTCNANTNNTNNGCTNNNNTNCNTATTNNNNNCTTCTNNTCNNNN
TTTNNNTACTNCNNTTTNTACNTNNCNNTNTNTTTNNCTCTNNANNNTTANNTNTNNTTTTNTNNTNTT
TTCNNNTANNNTNGTNCTNNTTTTTNANCTTNNNNNTTACNCTNNNTTNNTNTNNATTNANTANTNT
TNNANTTTANTNTNTTANNNANTTNNNTNNNNNNNATNTNNTNNTNTTTNNNTNNTNATNCCTTTNN
TNNNCNNATNTNTNTTNTNATTTNTTTTCN
NCGNCANNGNTCGCTACCGTNTANNNNTNNNNNNNNNNTNNNNNNNNNNNNCCCNCCCGCTCTCTCN
ATCNNCCACNTCTCCCTCCCTTCTTNCCNCNCCTCNNNNNANNNGNCNCGANGNGCAGTNCCNTGCCC
NTTTTNCACAAAAACTGGAGCTCCACGCGNGGGCGGCCGCTCTATATCTAGTGGATTCCNCGGNCTGN
NGGCAATTATTATTTCNTNCTTTATTTATACACCTTCGAATACACTCAATNTANNTATNATNGCCCACTT
CTCATCCCTATNNTCGNGTTCCTNTNCTTCNTCTTGNTCNNANACAGANCCCATTCTCATTCNATCNAC
NTTGNTTNTACTAGNTANTTCGNTNNNTATTCNTCATTATTTTNTATANTNTNNATANACTANATATNA
TTNTTAANTNCNATNTTGTCTTTTCTTCATCTTTNCCNTTTAANACNANTCTNTTTATTNCAAAATNTTANT
TNTATTTGAACANANTTNTNANTTATTNNNCCCTNCCTNCTATGTNNATATTCNATTNTTNATGAAACT
ACTCTTNANNTTCANTTNATTCNTCANTATNTANTNACNATTAATATNATGTNNCTNNNNTNCACNTAT
TNNATACCCAANTATCTTTTCATATCTCANACNNCNNTTTNATAATCANATCTATNTCTNTNNAATCTT
TNACAAGCTNNCNTAANNTCTANNTCATNATTATTTCTNNNCTGNTTANANTNNANTTNNATTNANNT
NCACCTTNCTTCAANTATNATNTCNATGTACNATANNNCNNNNCANNAAACTCNTTNNTNCTNTTCCN
NNCCTATTNCTAATCNCTNTAANTTCATNCANTNANNCTNNANTNTTANTNNTTNCTNNNTTANGTCTC
ATCCANCATTNTCTNCTNCTATTTNNCNACCNTCNTCNCCNTNTNTTCATANGATANANTATNTTCATN
NTCAAACATTCNNCCTNNTAANTTCTTATAACTNNNNTTATNTNAAGNNNNCCATGTTATCTNATCNAC
ANTTACNNTTCTTNTATATATATCTCNGTTTTANCNTNGCATNGNNAAGTNGCNTTTTATCTAGCNNTA
TNNNCTTATCNTNNTCATTNNTCNTANNTTANATCTGNCTTCNTANTATACTTCTCANATANGCATACA
TCANCTCNACACANTTCNNANCACTGTTATNNANCATANCTACNNTTATNCTNTATCTCTTNTNNNNCN
TTAACANNTACTCANTATTAACTNGTCATATCTATCNTNTNATTTNTATNNNNTCATACCNTATNNTCT
TANATCTATTNATTCTTANTGTCATTATTATACNNTTNTANAGNATAAATTTCNTACNTANCACTATTTA
CATTGTCTAGCGCGATNTACATTTNTCTTNTNCGCTNANNCCNCAGTCTTNTACTTANATNATNNTTNTA
TNCTACTATCCNNACTCTNTNNACNTANAATCTANTTNCATTNANNNTTNTCCCTCTNNGNCAATANAC
NNANANGTCNCATNCNANGNCTGNACTATAGACACACTATTCCGATNNNTTNTCATATCTCNTNTGAC
TANNTANCCC
TTGAGANCNTNNNNTGGNGNNGNANTNCANGGGGGAGTACGCTCTAGAACTAGTGGATCCCCGGGGC
TGCAGGCAATCCNGCANGAGGAGNNGNGCANCTTTCTTCGGTCGTGCCGANTCCGGGGGGCATGCTN
ACANNNCTCGGCTTCAACCNNTANTCGNGTTGCNNNNCGANCNCTAGNGNGGNNGANNTNCGGGGNN
NNGNNNTCTTTNNNNNGGTNANCNTGNGCNNCNTNATNAANTCANNGAANNNANANCGANGNGCGNN
NNGTNATNCNNAGTCCATANCNNNTGCNTTAGCCAGNNCCNNGNTNCTGGAGNANGGTNGNATNGCN
GNNTNNNATAGNCCNNTTNNNGNANAATAGACNNATCNACAGGGNNAGCNGTNGGTGANGNTNGGA
GAGNNNTANCATGCCNNNNGAGNTACNTGTTNACCNNGATACGGGACCNCGATNGCNNNTNNCCNTA
AGCNCNGNANNCNGCNGTCNNNCNTCTGNGCNNNTTTNTTNNGGCNCNCCNNTAGCCNATNTCNATT
GGANCAAGAGNCGNTCGANANTCNTNCANGGATCNNCNTNGCNNGAGNNNGNGCNNATCCANNNCT
GNNNTTGNNNNNCTGNNGNNCANANNACNNNTTNCGNANGTNNNGNNNAGTGNTGTNNNACNATTNN
NCNNTCGAGAANNNCGTNNGANCNATNTCGNNTNNNTNGGAGTNCCGGACNNTCCNGAANGNNNCG
GNNNCGGGATTNANANGANNNTCTTCNTNCNNNGAAGNGNNANCGNNTNGTGGGAGNTTGNNNCNN
TAGANCATNNNNNAAGNNANNNAAAAGGTTGCNGANACGTGCNAGNNTNNGGCGTGATACGCNNTG
GTAGGNCNNTNCTGGNTAGGAGNNNNNNTNNNTAANNTGATCANTNNCGATCNNANGTANTNAACNG
TNTNNNNNNTGTGCCGANTCGNNNNGGCGTNNNGNGGATGCNCNANGGGNANNCTGNCTGNATGCTA
NNNTNNNTGTCNNNANTCNGCNNAGCNNCCTNTCNCAGANCATGTNACNCTANCANTNATACATTGN
NATNTNAGACNNNAGCTATGGCGTNAGCNANCTANGCATCATNAGTCNTGACANNATNTNAGNNACN
GNTGCNANNGCNGGNAGTAGTCGNGTGCGTCCCCGNGCGCGNANANANTGTGGTNNTTNTTCCTCCTA
NNTCNGTATCGNCG
ANNATCTGGAGCTCCACCGCGGGGGCGGCGGCTCTAGAACTAGTGGATCCCCCGGGCTGCAGGANTTC
GANCNCTTNTNTTTTGAGGCCTCTGACAAAGCCGCAATGTTTNNGACTAGAAATAATTACCCAGGGAT
GATCATGACGTACTAAATACCANGACAGAGNCTANGAACTCATTATGAAACCATTNACTCGCAATGGN
NNNTGCCNATCTGNNACACANGTCCTGTCNTTTCATTCATCTCANATTATTTTGAATTCTTTGCAAATG
GCTGTNGAGNTTANATGGNGGTAGGGCTGTGAGCCATAAATCTGAAGCGTTGANAGCCTTGGGCCTGG
AGAGCCATGAANAGGGAAAGAAAAGANGGCNNGTCTCGAACTNAGCCAAGGACCNGATGGATTGCT
CGACCCAGACACAGAAGTGAAGCCTGTGTNCACGTGTCGCCGACAGACTGGAGTTTTTGGTGCTGAAT
ACAACANCGTGTTGCTGAAAAACCANGGGTTTNGGTGAATANAATGTTTTGATTTGATTGCTATGTAT
GTNCTACCAGTGTGTGGACCTTTGAAANATNATNNAGNTACCCCCCGATTNNTNANACNAAANAANN
NAAACTCCNTCTCTGGGGGGNGCCCCCNGNNNCCCTAATTCCGCCCTTTATANNGNNTNCCCCCNNAC
NNTNCCCTATTTCTTGCTTNNTCTCCTNNGNNNGCNNNTTATCATNTTCCGATTCATTACATTTATCTCN
GNNTGCTNCCCNCCTNNNNNCCTNCCNTTTTNCGNNCGNTNNTNCCCTNNNNGCTNNNTTTTCCNNAA
NCCTNNNNTCTGCTNNTCTATCTNCCCNGNNTCANTGNNTATCCNTTGCGCNTNCNTGTNNAATNTCTN
TANCTTANCTNGNCTTGCCATCNNGTCTNNTTGNCNNGNTATCAGTTTNTTTCGNNTATNCNCNNCC
TNNCTCGNTTTCNTNTGNANTATCTGTCTNGNNCNNTTTNCGTNANCATCTGCTCTNTTTGNGCANG
TNAGCCTATAGNGAGTNGTATTACAGTGGCGGCCCGCTCTAGAACTAGTGGGNTCGCCNGGCTNGNNG
GTTTGANAAANNAGACANCATNTGNCCNCGCNCNNGNGGNNGGGGGGANNNGGNGNANGNNNGGCNC
TTNNGNNGCGCAANGNNAGNNNAGNANNNNNGANGNAAAANCNNACNTTTTGNNCNNNGGANCNGA
TACANTTCTTAGGTGNACCCCTNTGTGGNCNGACCGGTGTTTCTCNACAGAGCTNNTGCCAGCTTANT
ANGCTGAACNCTGTCACTGCATGATNGGTGAANGTCNACGCTTNCGTCTNGAGANTGGTGACTCCGAG
CTANNAATTGCTGTCCNTCCGGGGAGCTCTNGGGAGTTCTNGNGCTNTNNNGAAANGGGCCCTATGAT
AGANGATNTGCATACTNCTCTCTGANCNCACTNNTGNCCCNTGCCACANANAGNTTTTTNTGAAACGT
GAGTGTATCANCCNCTNACCNNCTGTTCNTCNTCACAAATGCNNAGTTCCTNCAGNATTTTGGGCTACT
TTGATGCTCTTCGCCACNCNANNAGGNCCCNTGCAGGNGGAGTGGNCACCCACNCNGGACNCAANTT
TTNTGCTANGGGTTTCATTGANCAAACTTTGGAANGNGNNTGGCCNTGCANTTANTTCCNNACTGGGN
AAGANAAGGANAACTANCNTCCCCTTTTCNTGAAGANTGGNNNTTNNATATNNCTNCCGCCATTTGAA
CNTNCNATTTTNNGGNNAAAAGGGACNGCAANGGNCAAANAATNNTTNNTGTAANGTGGNNGNNCNC
GGNNNNNTAACANANNAAAAATCTNGNNCNGNNCTCNCTNNNGGGGNGGGNCNNNNCCGNCCCATN
NTTNTTTGTTTTTATGGGNCNCCTCTTTAAAACCNCNANATTCNNAAAGNCCNNGNNCCNTTNCCCNTG
NCNCGGCNNNTTNTTNCCCCCCCCCANTNNAGTCCNAGATNNCTTTTTTTCNTCTCCCNANANTCGTGN
CNNCN
TNAGCCTATAGNGAGTNGTATTACAGTGGCGGCCCGCTCTAGAACTAGTGGGNTCCCCNGGCTNGNNG
GTTGANAAANNAGACANCATNTGNCCNCGCNCNNGNGGNNGGGGGGANNNGGNGNANGNNNGGCNC
TTNNGNNGCGCAANGNNAGNNNAGNANNNNNGANGNAAAANCNNACNTTTTGNNCNNNGGANCNGA
TACANTTCTTAGGTGNACCCCTNTGTGGNCNGACCGGTGTTTCTCNACAGAGCTNNTGCCAGCTTANT
ANGCTGAACNCTGTCACTGCATGATNGGTGAANGTCNACGCTTNCGTCTNGAGANTGGTGACTCCGAG
CTANNAATTGCTGTCCNTCCGGGGAGCTCTNGGGAGTTCTNGNGCTNTNNNGAAANGGGCCCTATGAT
AGANGATNTGCATACTNCTCTCTGANCNCACTNNTGNCCCNTGCCACANANAGNTTTTTNTGAAACGT
GAGTGTATCANGCNCTNACCNNCTGTTCNTCNTCACAAATGCNNAGTTCCTNCAGNATTTTGGGGTACT
TTGATGCTCTTCGCCACNCNANNAGGNCCCNTGCAGGNGGAGTGGNCACCCACNCNGGACNCAANTT
TTNTGCTANGGGTTTCATTGANCAAACTTTGGAANGNGNNTGGCCNTGCANTTANTTCCNNACTGGGN
AAGANAAGGANAACTANCNTCCCCTTTTCNTGAAGANTGGNNNTTNNATATNNCTNCCGCCATTTGAA
CNTNCNATTTTNNGGNNAAAAGGGACNGGAANGGNCAAANAATNNTTNNTGTAANGTGGNNGNNCNC
GGNNNNNTAACANANNAAAAATCTNGNNCNGNNCTCNCTNNNGGGGNGGGNCNNNNCCGNCCCATN
NTTNTTGTTTTTATGGGNCNCCTCTTTAAAACCNCNANATTCNNAAAGNCCNNGNNCCNTTNCCGNTG
NCNGGGCNNNTTNTTNCCCCCCCCCANTNNAGTCCNAGATNNCTTTTTTTCNTCTCCCNANANTCGTGN
CNNCN
TTGAAAACATCTGGAGCTCCACCGCGGGGGCGGCCGCTCTAGAACTAGTGGATCCCCCGGGCTGCAGG
AATTCGNCANGATGCNTTTCGNTACCGNCNNACTGGANGGNNNACNNNGNNCAGNNNCCTTCNTATA
NTGNTTNNTANTAAAGGNNTANATGAGTNCTTTNNCNTNATATACCTCAGNGTACACNCTNNAGTCCC
AGACATTNTNACTTCTGCGTTTTNTCNNTTGAANTATNTGCTATTNGCTTTCGAANTTATGCTACGACN
TANNAACAGACNANCTGCACTNNTTTATNTNACAAACCNNNATCCGNGNNNNTATCNTGCNTTTNNNG
GNTTACATNCANCNGCCTCTTTATATTCTGNCCCTCACACTGCANNGNTNNANNTCTTGANACTGGNC
GGCNCNTATNTGNANGCTCTGGAANGNNCATCNNNCNNATCAAATNANATCCCNTNNNTANTCCNAA
GNNTTTNTCCTTNNAANACNATAGCACNCCNTTTNCCGNTGGAAGTTTTNTNGAAAAAAATTNCCTTNC
NNNAGNTNTTANAACCCTNTNCNNAAATTTNNTGGNTNTNGNTTNNAATTNNNAACCNCCTTTAATTA
CNNGNTNTCTTANATTNNACCGGNGNTTTTTNTAAACCATTTTGANTNTCTTNTGGTTAAAATTCCTTT
NTCCGNAAAACNCCCTTTTNTAAANTNAANTGGNNNANTTTCTTNAANNAAAACACAANNTTTNTNTT
TTTTNGGGGGGNNNNCNCNNCNAACCANACNTCTNNAANTTTTTTTNCTTTNAAACNAAANAATTCNT
TTTNTTNNCTNNTATNTNNTTTNTTNANTTTAAATTTNCCCNNTNTTTNCNCTTTTNTATTCTNCTTNAN
CTNAATATGTNCTCNTCNCNTCNNTTATCNTNNCNCCNATTTTTNNNTCTCCCTNTTNTCTTCNCNTATA
NNTTNTNNGNCTCTGTCNTTANTNTNANTNTTCTTTNNCANGCTTTTTTNCTTCANTCTNATCTNCCCNC
NNACTNATCTTTNANNCNATNNNNNCTCT
TTGAGGCTATNNGGAGCNCGCATNNCNGNGGCGGCCGCTCTAGTGGCTAGTGGATCCCCCGGGCTGCA
GGAGNTAAGGCTGAGAAGNCNGATACTAANNNGAAAAANCCTGTGCCANGANGGCTGANNCTGGCN
AGAAGGCTAAANAGGTGGAAANGGTGACNAANGTTANGAAGGGNAAGCCCCACTGCAGCCGAAATC
CTGTCCTGGTCAGAGGAATTGNCAGATATTCCCGATCAGCCATGTACTCCAGGGCCTTGTACAAG
AGAAAGTNTTCAGCAGCTAAANCCAAGGTCGAAAAGAAAAAGAAGGTTCGTGTTCTTGCTACTGTCAC
AAAACCAGTTGGTGGAGACAAGAATGGTGNACCCGAGTGGTCAACTTCGCAAANTGCCTAGGTATTA
CCCTACTGGAAGATGTGCCTCGAAACCTGTTGAGTCATGCAAAAAACCCTTCAGTAAGCATGTGAGGA
AGCTGCGTGCCAGCATTCACTCCTGGGACCATCCTGATATTCCTACTTGGGCGCCACAAANGCAAGAN
GGTCGNTTTCCTTNAANCAGCTGGGCAGNGGCTTGGTTACTTGGGACTGGGNCCTTTATCCCTCAATCG
AGTTCTTTGNGGANAACACACCANAAAATTGNTATTTGCCNCCTNCACCCAAAAATCGNTTTTTCAGGN
GGGGGNGNAAAATNCCCANAANATTTTTACTTGANCCTTTACTTTAAANAAANAAAAAAACCTGGGTT
NAAACCCCANACCCCCNGGGNGGGCGGANATNNTTCGACCCCCGGGGNGNGNGGGNAANTTTCCCGG
GATTTCCCCNGGCCNCGCNCCANGGGTNCANTCCAAAAAAAGCTTGGGGGGCCTNCNAAAAATTNTN
GNGGAANNAAAANAAAAACGTNTTCCTTTAANGTTTCCANGGGGNTTCCCTTCCCCTNCCGGGGTTTN
GCTTTCCCCCAAAAGGGGAANTTTTTTCTCNCCAAAAGGAGGGNGTNN
TTNANNACAANCTGGAGCTCCACCGCGGGTGGCGGCCGCTCTAGAACTAGTGGATCCCCCGGGCTGCA
GGCCAGATTTCCTTTTCTGTTTTTGGATTGCTGGCAGCTTAAGGGCCAAAGAGGGTGGTCATAGTGTTA
GTTGCTCAGTGGTGATGGACAGGGAAGCCTGGTGTACTGCAGTCCATGGGGTCACAAAGAATCAGAC
ACAACTGAACTCAATTGAACTGTGTCTCTTTACAACCTGATAGACTGTAGCCCACCATGCTCCTCTGGC
CCTGAAATTCTCCAGGTAAGAATACTGGAGTGGGTTGCCATTTCCTTCTCCAGAGGATCTTCCTGACCC
AGGGATCAAACCCAGGTCTCCCTCATTGCAGGCAGATTCTTTACCATCTGAGCCACAAGGGAAGCCCA
AAATGGTTGGCAGGCAGGCTGAAAACACCACAGACATTTCTTGAGTTTATGTGCCTTTTAGTGTAAAG
GGTCAGAGGAAAGAGAGTCATTTTGAGAGAGCAAAATGACTTTTTCTTTTTATCTTCCTACTCTCTTCC
TGAAGTGCCATTTGAGACTGCAGTAAGGTAAGGGAAAGAATGCTTTCCTTCATGAAAAGAGGCAGGG
GATCAAATTGATGTGTAACTTGGCCTAATGAAGGACTCCCACGCCGATTGTGGNTGACTTGCCTGCCT
GCACANGTGGTGGAGGNAACGATGTGGTTTAACCGNGGTGAACTCTGATAATAANCGGCTGGANCCG
AAATCCCGCCGGGAAAGCCTGGGTCTNCCTCTAACCTTTAATTTGGGGGCCTCCCANAACTGGNCCAGG
NAATTGGGCTTCNCGCCGGGTTCAAAAAGNCACCGNGANCCAAAAACTTTTGAANGGGTTTGAAAAA
CCTTTTTGGNCCAAACCTTGNAAGGGGANGGCCCCTTCCCCAAAANCCTTGTTGNAACCCNCCAAGGG
AAANCCCANGGN
TTGANCANAGCTGGAGCTCCACCGCGGTGGCGGCCGCTCTAGAACTAGTGGATCCCCCGGGCTGGAGG
GAAGAACTCTGATGCAGAGGTTGCACCTTTTCCCAGATGAAGATATACCAGNAGATATTCTTAAGAAT
TTAACAGAAGAGCTTCCTCAACCACGAAAAGTGCCCAGACGTCTAGATGAGTACACACAAGAAGAA
TAGAGGCTTTCCCAAGAGTTTGGTCTCCACCTGAGGATTATCGGCTGTAGGAGGAAGAAAATCAGA
AAATAACGGTGTGGTGCTTGAAACTCCTTCCTCAGGGCAGGATGAAGCAATAGCTGCCTTCTGGAAGG
AAGCCTGCTCTGGGGGCTGAAAGTTTGTGGGGAAAGGTCGGGAAGGGGTATCCCTTCATTAGGAAATT
GCAGTANGATGTCATTTTATAGAATTGNTTATTGTATAGTGCTATTTGAATGTTGGAAATGATAAGAA
TAAAACTGCGCCTTACTTCAAAAAAAAAAAAAAAAAAAAACCTTGGGGGGGGGCCCGGCCCCNAATT
TCCTTNTANGGGAGNTNNNTANNNANNNNNNGGAANGNAAAGANTGCTTTNNTTNTTGANNNNCNGC
CTGGGNTNAANTNNNNCNTNTNTTGNCCTNGGGANNGACNNCCNNCCNNTTGGGGTTGNCTNGCCT
TNCNNCCTNNNNNGNGGNNGGAACCATGGNCNNNCCNNGNNNNNCNNTNGNNNNNNNNGNNNGGCC
CNNNANNCNNCNGGNNNNNCNNNNNNNNNCNNNANNNNAATTNGNNNGNNNNNNNNNNNNNNCNN
NNNNNGNNNTNNNTNNNCCCNNNNNCNNNNNNNNCNNNNNCNNNNNNTTNNNGGGGNNNNNNNNNN
NNNNNNNNNNNNNNNTNNNNNNNNNNGNCCCCCCCCC
AAAANCTGGAAGCTCCACCGCGGTGGCGGCCGCTCTAGAACTAGTGGATCCCCCGGGCTGCAGGAAA
TTCGATATCAAGCTTATCGATACCGTCGACCTCGAGGGGGGGCCCGGTACCCAATTCGCCCTATAGTG
AGTCGTATTACANNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNTNNNNNNNNNNNNNNNTNNNNNNNNNNNNNNNNNN
TNNNNNNNNNTNNNNNNNNNNNNNNTNNNNNNNNNNNNNNNNNNNNNNNNNNNNNNCNNNNNNNN
NNNNNNNNNTNNNNNNNNNNNNNNNNNNTNCNNNNNNNNNNNNNNNNNNNNNNNNNNNNNNNNNN
NNNNNNNNANNNNNNNNTNNNNNNNNNNNNNNNNNNNTNNCNNNNNNNNNCNTNNANNNNNNNNN
NNNNNNNNNNNNNNNNTNNNNTNNNNNNNNNNNNNNNNNNNNNNNNANNNNNNNNNNNNNNNNNN
NNNNNNCTNNNTTNNNNNANNNTNNNNTNNNANNNNNNNNNNTNNTNNNNNNNNNNNNNNNTNNN
NNNNTNNNNNTNNNTNNNNTNNNNNNNNNNNNNNNTNNNNNNNNNNNNNNNNNNNNNNNNNNTTN
NNNNNNNNNNTNTNNNTNTTCNCCNNNNNNNNNNNNNNACNNCNNNNNNNTTTATNNNTCNTNNNN
NNNTNNNNNNNNNNNNANNNTTNTNNNNTNNNNNNNNNNNNNNNNNNNNNNANNNNNNNNNNNNN
NCCNNNNNTNNNNATNNNNNNCNNNNNNNNNCNNNNNNTNNTNNNNTNATNNNNTNNNNNNNCNNN
NNNNNNNNNTNNNNNNNNTNNNNATNNTNNTNNNANTCCNNNNAGCNNNNTNNNNTNNNNNNNNTN
NTNNANNNNNTCNTNANNNCNNNTNNNNNNNNNNCCNCNNNNNNNNNNNNCG
NNCAAANCTGGAGCTCCCCGCGGTGGCGGCCGCTCTAGAACTAGTGGATCCCCCGGGCTGCAGGAATT
CGGCACGAGGAGAGGTGCAACTTTCTTCGGTCGTCCCGAATCCGGGTTCATCCGACACCAGCCGCCTC
CACGATGCCGCCTAAGTTTCGACCCCAACGAGATAAAATGTACCTGAGGTGCACCGGTGGGGAAGTCG
GTGCCACGTCTGCCCTGGCCCCCAAGATCGGCCCTCTGGGTCTGTCTCCAAAAAAGGTCGGTGATGAC
ATCGCCAAGGCAACTGGTGATTTGGAAGGGTCTGAGGATTACAGTGAAACTGACCATTCAGAACAGAC
AAGCCCAGATTGAGGTGGTACCTTCTGCTTCTGCCTGATCATCAAAGCCCTCAAGGAACCACCAAGGG
ACAGAAAGAAGCAGAAAAACATTAAGCACAGTGGAAACATTACTTTTGATGAGATGGTCAACATTGC
CGGCAGATGCGGCATCGGTCTCTAGCTAGAGAACTTTCTGGGAACCATTAAAAGAGATCCTGGGGACC
GCCAGTCTGTGGGCTGCATGTTGATGGCCGCCACCTCATGACATCATTGATGATATCAACAGTGGCGC
AATGGAATGCCCGCTAGTTAAGACTGCAAAGGAAAATAAANGGACCATTTGACACCAAAAAAAAAAA
AAAAACTNGNGGGGGGGCCGGGNCCCAATTTCNCCCTTTAGGGGGGNNNNNNNNTNNANNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNC
ACAAAANCTGGAGCTCCACCGCGGTGGCGGCCGCTCTAGAACTAGTGGATCCCCCGGGCTGCAGGAA
TTCGGCACGAGGGCTGCCTGTTACAATAATCAGCAGGTGTTCAAGGGGGTGGTGAAGATTCNAAAAG
GGCAAGCGGTCACCCTGATGATGGATGCCACCAACATGCCAGCTGTAAAAGCCATAATACACCAGTAC
ATGGAAGAGATTTATCACAGAATTCCCAACTCTGACCCCTGTTCCACTAAAACACAGCAGATCATCTC
CACCATCAGGACACAGAATCTCCCCAACTGTCAGCTGGTCTCGCGGAGCCACTACTGGCCCATCTACC
TGTCGTTCGTCATGCTCCTGGCGGCCCTGAGCTGGCAGTACCTGAGCACCCTGTCCCAGGTCACAGAG
GACTATGTTCAGACCGGGGAGCACTGACTGGCTCGGTCTGGAGACTGAACGCCCCTCCTCCCAAGCCC
CTATCTGGGAAACAGACTGACCTTCTCTTCAGGGATGGATGTGGGGTCCTTCTCTTTTTTCCCCTACTGT
NTTAATCCCTCAAAGAGTACTGTGGGCCTGGACCTTTANAAACTGTGACCTGTGGTGGAGAAAAAGAT
AGGATTAAAGGGAAAGGACAGCTCAGCCACCTGTACTCACCTGTGCGGGGTGACTGACGCCCGAACG
TTCACGGNTGCCATCAAGGAAAGGGCTGCATCCGGGGCTTGNAGAGGAAGATCATAGTGTGAATACA
GGGCTANAGTTACAANTAAATGTATTTAATGCCGAACCAAAAAAAAAAAAAAAAAACCTNGGGGGGG
GGGCCCGGGNCCAATTTNCCCCTTTATNGGGGGGNGNTTTTNAAANNNNNNNNNNNNNNNNNNNNNNN
NNNNNNNNNNNNNNNNNNNNNNNNNNNNNNNNNNNNNN
TTGAGNCCTTTNTAGTGNNGTCGTANTTACAGTGGNGGCCGCTCTAGAACTAGTGGATCCCCCGGGCTG
NAGGAAATTCGGCACGAGGAATNNNTNTATCTGTGATGGTGGCNNGAAGACCGNGNNTGTCTTGCCA
NAATGNNTTANATANANCAAANNTGTNNCTGCNCNTACANGNNAATACNTANCTTNTGGGCCNAGGG
GGAGTGNATNCGNGNNGAANTCNCNCTTGCNCANCTTANTGATCANGTGCNANNCACNNNNTANNAG
NCCATGATCNTCCTGNCCGGGACTCNGGTGGATNNNANGNCNNNNTGAGGCCANCGANNNGANAGCN
GNCTNAAGCACTTTATNCAANCTCTCTGGGGCTGTGGNTATGCNTNTCATNATTGGTATTACGNCCCG
NTCTTGCNCCTTNNGNTTNCCTNGTANANNCACCTACCAGGNNNGGAAGACNAGTCNGNATTAGNTN
GNTTCNGGTNGGNAATCNNTGATATTTTNNNTNGGNNNAAGGGCGTCCTCCCNNCATTTTTGNTNNTT
NANNACTACTNGAGNACNNCTNTAACTNNTTATTTTTTTAATTTTCCNTCTTNTNNCAACCCTTGCNTT
GGNCAGCCCTTCNCNTCTANTNTNTNATTNCTTTTTNNTGGATCCTCANNTTNTTTTNNCNATATNNTG
CNNCGNCCNCTTCTTCNTNNNANCTNGCCANTNNNTTNTCTTTTTNGNNTNANTNCGTTTNACNCCCTCT
NNNNANATATTNCTNAANGGGGNTNNNGGTTNGNNTNTTNNTTTATTTATANANGNNTCNGNGNNTTNTAN
NTANNNCCCNANNNNTCNATAAGAATCCNNNTGATCTNTCAGNNANCTCTCGTTTCCTCTTNGNNNNN
CCCCNCAATAGTNNNTNATNCNATNTAGNGNCNNGNANCATTGTCNCTTNNNGTNCTCTNAGTCCNTC
AACNATGATNGTCTCNNGGNNNATTNTNTATCTNTCCCTNTNANAATTANNNTAGNAGATNCNNNTNT
CGNNNCACNTAATAACTNGATGTNGCTCCTAGGATTTCNCTTTGNGCNCTNCG
TTGAGNCCTTNTAGTGNNGTCGTANTTACAGTGGNGGCCGCTCTAGAACTAGTGGATCCCCCGGGCTG
NAGGAAATTCGGCACGAGGAATNNNTNTATCTGTGATGGTGGCNNGAAGACCGNGNNTGTCTTGCCA
NAATGNNTTANATANANCAAANNTGTNNCTGCNGNTACANGNNAATACNTANCTTNTGGGCCNAGGG
GGAGTGNATNCGNGNNGAANTCNCNCTTGCNCANCTTANTGATCANGTGCNANNCACNNNNTANNAG
NCCATGATCNTCCTGNCCGGGACTCNGGTGGATNNNANGNCNNNNTGAGGCCANCGANNNGANAGCN
GNCTNAAGCACTTTATNCAANCTCTCTGGGGCTGTGGNTATGCNTNTCATNATTGGTATTACGNCCCG
NTCTTGCNCCTTNNGNTTNCCTNGTANANNCACCTACCAGGNNNGGAAGACNAGTCNGNATTAGNTN
GNTTTCNGGTNGGNAATCNNTGATATTTTNNNTNGGNNNAAGGGCGTCCTGCCNNCATTTTTGNTNNTT
NANNACTACTNGAGNACNNCTNTAACTNNTTNATTTTTTTAATTTTCCNTCTTNTNNCAACCCTTGCNTT
GGNCAGCCCTTCNCNTCTANTNTNTNATTNCTTTTTNNTGGATCCTCANNTTNTTTTNNCNATATNNTG
CNNCGNCCNCTTCTTCNTNNNANCTNGCCANTNNNTTNTCTTTTTNGNNTNANTNCGTTNACNCCCTCT
NNNNATATTNCTNAANGGGGNTNNNGGTTNGNNTNTTNTTTATTTATANANGNNTGNGNGNNTTNTAN
NTANNNCCCNANNNNTCNATAAGAATCCNNNTGATCTNTCAGNNANCTCTCGTTTCCTCTTNGNNNNN
CCCCNCAATAGTNNNTNATNCNATNTAGNGNCNNGNANCATTGTCNCTTNNNGTNCTCTNAGTCCNTC
AACNATGATNGTCTCNNGGNNNATTNTNTATCTNTCCCTNTNANAATTANNNTAGNAGATNCNNNTNT
CGNNNCACNTAATAACTNGATGTNGCTCCTAGGATTTCNCTTTGNGCNCTNCG
TNAAAAAAGCTGGAGCTCCACCGCGGTGGCGGCCGCTCTAGAACTAGTGGATCCCCCGGGCTGCAGG
AATTCGGCACGAGGGCGGCGCCCAGAGCCGGNTCNGCGCGTNNACTGCCCAGAGTCCGCGGCCGNNG
CGCCCCGAGAAGCCAAGCANCCATGGCCTATCACAGNTTCCTGGTGGAGCCCATCAGCTGCCACGCCT
GGAACAAGGACCGCACCCAGATCGNTATCTGCCCCAACAACCACGAGGTGCACATNTATGANAAGAG
CGGGAACAAGTGGGTCCAGGTGCACGAACTCAAGGAGCACAACGGGCAGGTGACAGGCATGACTGG
GCCCCAGATAGTAACCGTATCGTGACCTGTGGCACAGACCGCAACGCGTACGTNTGGACNCTGAAGGG
CCGCACGTGGAAGCCCACGCTTGTCATCCTGCGCATCAACCGCGCCGTTCGNTGTGTGCGCTGGGCCC
CCAATGANAAAAAGTTCGNTGTGGGCAGTGGCTTCCGGTGTNATCTCCATCTGNTATTTCNAGGAGGA
AAATGACTGGNGGGNGNGCAAGCACATNAAGAAAGCCCATTCCGTTCCACCGTCCTAAGCTTGGACTT
GGCACCCCAACAGNGTGCTTCCTGGCCGCCGGNTTCTGNNANTTNAAATGCCGGATNTTTTCAGCCTA
CATTAAGGAGGNGGGNGGAGNGGCCAGCACCCCACCCCGNGGGGGTTCCAAAAAGCCGTTCCGGGGA
ACTTGATGNTTTGAAATNCCAACAATAACCTTGCGGTTGGGGNGCATGGGGNTTTGGTTTTTNGGCCA
AACGGGAANTNNTGTAANCCTTGGGGTNAACCNCCAAAAAGNACCCNGGGGGCCTTGGGTTTGATGC
TTGAANAAAAAAAAAGGGCCGNNTNNGNAAANTTTTGGCCTTTTGAAAACCCTTTNCCGTTGGTTGGCC
GGTAACCTTTTTTTAAANGGGGAAGGAGGNT
TTNAANCNCNATCTGGAGCTCCACGGCGGTGGCGGCCGCTCTAGAACTAGTGGATCCCCCGGGCTGCA
GGAATTCGGCACGAGGGTTTTATAAATTTAATATATTTATCAAGCCAAATGTGCAGATGCTAACTGGA
CGCTCTGGAGAAGTGGGCCCAGGGACGCCCNACCCTGTTCTCTNTTCAGCAGTGGGAAGGCCACATAT
TTCAGTGGCCACAGCCACAGGAAGCTCAGATTCTCTGGCTAAAGGTAACACTTTGCCCTCCTGTGCTCC
TCTTANCTTCCCGCCACCCCCAGGTAGGGGGCGAGAATGGCACTGGAAAGGCCTGGCCCTAAAGTATA
GTTCTTANAGCAAGGGGAGCTTANACCTCAGGCGCCCCCAGGGAGGCCTTGAGACTGGGCAGAAGGG
TTCTGGTTGCTAGAGCCTGTGTGCAAAGGGGTGCCAGCAGCCCCCCTGAGCTAGTGCTGGGAGGACTA
ATGGTGGGGGGGGGGGCCTGNTACTCCCCTNAGTCCCCTCCTCCNTGTTGACATNTGGGGCTTTGACC
CTTTNTTTTTTAATCTACTTTTGCTAAAAAGCNTTTAATAATAATAATAANGGGACAAAATGCAAACCT
TGTTTCCCTCNCCTTTTGGGCTTNTNGGAATGTCATCACGTCCANTTTGTTGGGTTTGTTTCCACTTGTT
AATAAAGCCTTTGAAACCGTNAAAAAAAAAAAAAAAAAAACCTTGNGGGGGGGGCCCCGGANCCCA
AATTNCCCCTTAAANGGAGGNNNNAAAAAANNNNNNNNNNNNNNNNNNNNNNNNNNNNNNNNNNN
NNNNNNNNNNNNNNNNNNNNNNNNNNNNNNNNNNNNNNNNNNNNNNANNNNNNNNNNNNNNNNN
NNNNNNNNNNNNNNNNNCCCCCCNNNCCNGGNNNNNCNCNNCNNNAAANGGNNNNTTNTNNCNNNN
NAAAANNNNNGNNNN
TTNANAAAGCTGGAGCTCCACCGCGGTGGCGGCCGCTCTAGAACTAGTGGATCCCCCGGGCTGCAGGG
TGAANACCACCGNTNNNNCTAACTAAGCCANAATGGGAAAGGNGAANACCCNNATCCANATCNNTGTCATT
GGGCACGTAGATTCANGGAAGTCTACCACGACTGGCCATCTGATCTACAAATGTGGGGGGATCGAGAT
AGAGAACAATTGAAAAGTTCGAGAAGGAGGCTGCCGAGATGGGAAAGGGCTCCTTCAAATATGCCTG
GGTCTTGGACAAACTTAAAGCTGAACGTGAGCGTGGTATCACCATTGATATCTCCCTGNGNAAATTTG
AGACCANCAAGNACTATGTTACCATCATTGATGCCCCAGGACACAGAGACTTNATCAAAAACATGATT
ACANGCACATCCCAGGCTGACTGTGCTGTCCTGATCGTTGCTGCTGGTGTAGGAGAATTTGAAGCCNN
NNTCTCCAAGAACGGGCANACCCGTGAGCATGCCCTTTNGGCTTACACCNTGGGTGNGAAACAACTAA
TTGTTGGCGGTAACNAAATGGATTNCACTGAGCCACCCTATAGCCAGAAGAGATCCCATGANATTGTT
AANGAAGTCAGCACCTATATTAAAGAAAATTGGGCTCANCCCCGACACAGTAGCATTTGTGCCAATTT
CTGGGTGGGAATTGTGCCAACCATGCTAGAAACCAAGTGCTAATATGCCATGGGTCAAGGGGATGGA
AAGTCACCCGTNANGGACCGGCNATGCCAGTGGGAACCCACCCCTGCTTGAAGCTTNTGGAATGGANT
TNTGGCCANCCAACTTGGNCCAAATTGAAAAAACCCTTGCGTTTTGCCTTTTNCANGGAAGGGCTAAT
AAAAAATTGGGGGGGATTGGGACCTGGGCCCCTTTGGGGGTCCGGGGGGGGAAAANTGGGTGTTTTTC
NAAACCTNGGNGTNGGGGGG
ANAACCGCTGGAGCTCCACCGCGGTGGCGGCCGCTCTAGAACTAGTGGATCCCCCGGGCTGCAGGAAT
TNGATACNAGGNNNNTNGATNCNNATTGNNTGGNAGGAGACACCCNNNCTTNTTCATGNNCATCTNN
NTNNGAGTNNCTCNGTCCATTCTTGCNTNCCGCATGCCTGTACTTTGACTGACATTCTGTGTNGNAAAN
NTGNNCAACCACTGTGGGGANCANCCCTACTGANGGAANANTGNTATANGNCNATNCCATAACCTTA
NTGACNNGGNTTATNNATCNCCAANNCTACTTGGGTATGGNAGTNGACTAGATGTNTNNTAACTTNTG
AGTATTNTATTTNGANGCTTGGCTNACATATAGACTTAAACTGTCAGCTTGGANNNGTCTATTNCTTA
AACTTTGNAANATATATATATATTTCTTGNTCTTCCATTGAATGCAAATTGATAGACAAAGATGTACNAT
TGCTCTGTTGTNTGTTNGGATTATGAACGAAATGGTTGTGACCAATTTGGNGGAAGGGACACACACCA
ANTCCTGGGGATGGNTATGTNTANNNTNCTTGGCATTGGCNTAAATNNTTGANAGTTCCCNTTCCCATT
NACACTCCCATNATGCTTTAAANNAAAAAAAAAAAAAANANNTNNGGNGGGGGGGGCCCCCTGACCC
CANATTTTGGACTTTTNTTGGAGNGCTTGTTTTAANAANTTGGNGNAAANTNTNNTNTNNTTCTTTTTT
GGGNGGAANCCCNTNCTTCCCTTTTNTTCCGNTNTNAAATTTTTNTGANCNTNAAAGGGNGACAAAGA
NNAAAACNCCNNNTNTTTNNGTTTTTTNTTTTTTTCCNCCNCCCCCNCTTNNNNTTNTTNNCNTTTTNNC
ANNGGNNNTTTTGGGNTTTTTTTTNNAGNNNTTNNNNAAATTNTNNTNTTCTCTNCNAAANAATCNCT
NGNTNNNTTTTTTCTCTNCNNGAAAANAANANT
AAANCTGGAGCTCCACCGCGGTGGCGGCCGCTCTAGAACTAGTGGATCCCGCGGGCTGCAGGGGAGG
GCNCGAAATTTGAGGTGATGAACTCCTTCGAACTGCTGTCCCACACCGTGGAAGAGAAGATTATCATT
GACAAGGAATATTATTACACCAAGGAGGAGCAGTTTAAACAGGTATTCAAGGAGCTGGAGTTTCTGG
GTTGGTATACCACAGGGGGGCCGCCTGACCCCTCCGACATGCACGTCCATAAGCAGGTGTGCGAGATA
ATTGAGAGCCCTCTGTTTCTTAAGTTGAACCCTATGACCAAGCACACAGATCTTCCTGTCAGCGTCTTT
GAGTCCGTCATAGATATAATCAATGGAGAGGCCACCATGCTGTTTGCTGAGCTGACCTACACTCTGGC
CACAGAGGAAGCGGAACGCATTGGGGTGGACCACGTAGCCCGAATGACAGCAACAGGCAGTGGAGA
GAACTCCACTGGTAAGGGTGGCTGCAGGGGCTTCCCTGGAAGTGGGGGGNGGNGNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNC
NAAAACTGGAGCTCCACCGCGGTGGCGGCCGCTCTAGAACTAGTGGATCCCCCGGGCTGCAGGAATTC
GATATCAAGCTTATCGATACCGTCGACCTCGAGGGGGGGCGCGGTACCCAATTCGCCCTATAGTGAGT
GGTATTACACCTGATNNNNNNTCAACNANANAGNTTGTTGCCTANNGNNATNACTNNGCNNNTACNN
NGGANCATATTNTATTTACNNAATNCCNAACTNNNTACNNNTGCACNCTANAANCACATANAANTTCAT
NTNACNATCANNACNCAAGANTCNCACTTCNNNTTNNNTCTNTNGNTNANNCNGNGNAAATANTCAN
NTNNTAANANGATNTNNTTTAATNTNNANTNCNTGTCCANCCNTTNTTTTTNTANTNANNNAATCTNA
TNCNTTTNCCANATCNCTTNANGTTTNNTNNTTNANACCTTGNNANCATNNNANNGCCTTGNTTNTTNT
TNACTTGNNNTCNTNTCTTGNAACAATTTNCTANTATNGATAANNANANTTTNTCTTTCTCATTTNNAGNN
NTGATGTNNNCNTTTTATNTTTTTTTGNNGTANNNNNNTNATTNNNTNCANTAANNTCNTNGTCNCC
NCGNTNCTTTNTNAANNNNCNAANCNTNNTCTNNNNNNTNATTGATCNNNNCTCTNTGGCNANNTNG
ACTTNCNTANNCNNCCTNNTNCACCTCNNGGNGTCTNTNTTNGANATNACNCANGANACTGTTNTNAC
NCTATATCTTGTNCNCCTNNGANTTCNCNNTATANANCNCNATTCNATTTTTNTCTTTNNNCTANA
NATNCTTACCNTNCTNTNACTNATNCTNATTNTTTTNATACTTTTATTNANNTNTTNATCNTNCCGACCT
NNCTNTNGGGCCTCCNTNTAAAACTNTCNTATTCCATNTNNANNATCTNCCTNTCNTNCTTTCAATTAN
TTNTNNTNCCACCTANTTNCNNNTNCNTTNTCCCNNCATTTTNNAGCTTCCNACTNCACCCTCTNTTCTT
CCNCATNACNANNTNNCACCCCTTNTNACTCTCTNNCACTANTCCCCTCCCCN
NNNNATCTGGAGCTCCACCGCGGTGGCGGCCGCTCTAGAACTAGTGGATCCCCCGGGCTGCAGGAATT
CGGCACGAGGGTAACACTCTGTTTACTGGAGCCAGCTCAGCTCAGCGGAGCAGATATAAACCGAGAAG
GGCAAACTCATCGAGAGGGCGTCTTCGGGACCATGGCGAATGGATATACATATGAAGATTATCAAGA
CACTGCAAAATGGCTTTTGTCTCATACCGAACAGCGACCTCAAGTGGCAGTGATCTGTGGTTCTGGGTT
AGGAGGTCTGGTTAACAAATTGACTCAAGCCCAGACCTTTGACTACAGTGAAATACCAAACTTTCCAG
AAAGTACAGTGCCAGGTCATGCTGGTCGACTGGTGTTTGGGATCTTGAATGGCAGAGCCTGTGTGATG
ATGCAGGGCAGGTTCCACATGTATGAAGGCTATCCGTTTTGGAAGGTGACATTCCCAGTGAGGGTTTT
CCGGCTTCTGGGTGTGGAGACCCTAGTGGTCACCAACGCAGCTGGAGGGCTCAACCCCAACTTTGAGG
TTGGCGATATCATGCTGATCCGTGATCACATCAACCTACCTGGTTTCAGTGGTGAGAACCCTCTTCAGA
GGGCCCAATGAGGAAAGGTTTGGAGTTCGTTTCCCTGCCATGTCTGATGCCTACGACCGGGATATGAG
GCAGAAAGCTCACAGTACCTGGGAAACAAAATGGGGGGAGCCAAAAGAGAGTTACCAGGGAAAGGC
ACCCTTTACCGTGGATGTTGGGGGGGGTCCCCAAATTTTTTGANAACTGNNGGGCANAANTGGTCCCCC
TGCTTGCCGGGAAATCTTAAGGGCCANAATGCTTGGTTGGNCATTGAAACACCAGGTTCCCANAAATT
TNNTATTTCNNCAAGAACACTTGNGGGACCTTTCGAGGNCNTTGGCTTTCTTGACTTCCATANCAAAAC
AANGGNC
GCCTATAGTGAGTCGTATTNCAGTGGCGGCCGCTCTAGAACTAGTGGATCCCCCGGCCTGCAGGANTT
CGGNACGAGGAACCNCTTTATCNGTGATGGTGNNNGGANAACCNNGANGGTCTNCCCANAAGGGNTA
GAANTANCNAAACTNGNGGTTGTNCGACCCTTGATTCNAAACNNTTTNGGNNNAGGGGGNANNNNACG
ANANGNAANNCCNACTNNNNANTTTNATGNTNAANTNCCAAACNCCNANTCNNAAACCCANAATCTT
CTNGCNGGGAANTCGGNGGGCCNAATGAANGGNGNNCCCCTTNACCGNGAANGGTTTAACACACTTAT
ATCCAANCCCCTTCGNGGTGTGNGGAGAGCAAANANNGANATGTGNTGACACNCCCACGTGNTATCG
CGCGCNGATATATGTGCACGANCACGANNCCATGTGAGAAANTTTGGTTTTNGAAAAGCGANNGAGA
CNCNGTNGCTCTTATNAAAGGGGGGCACNCNCNNNTGTGNTTTTTACACAAAAANTCTCACTCTACNN
CCTTTTTATAATATGGCCCCCNCACCTGGTTTGNNAACCTACTANGGTCAANNGTNTTTNNTANTGGTG
CGGCCNACCTTCTTCGNCNTTATACTANCTTNNGACCTNTTGTTGAGACCCCCNAAATNGGTTNATTCC
TNGGNGAANAAAGCANNCCTNCTGNTAATTATTCCTNCGAAGGATGGGTNCNTCTTGTTANNCCCANC
CAAAAAAAATTGGGGTGGGNTTTCTTGGGACCNCCACNNANGGAAANAAATGANGTTNCNTTNNTGA
CCTTCTTTTNTTCTTNNTGCCTNCCGGAAGCTTNATTAACTTTTTTTNTGGGGNACGNAGNTTAATCAA
CCNTTNNNNGNGCNCNGATTCNNTTTATGGAGGGAAAAAATTCNTTNNNTAAACCCCTNCCCNNNCNN
TNTGTTTTGCACAAAATNNTTNTGCNGGCGGTNTTTNCCTTTNNNNCGGGCCTTN
GCCTATAGTGAGTCGTATTNCAGTGGCGGCCGCTCTAGAAGTAGTGGATCCCGCGGCCTGCAGGANTT
CGGNACGAGGAACCNCTTTATCNGTGATGGTGNNNGGANAACCNNGANGGTCTNCCCANAAGGGNTA
GAANTANCNAAACTNGNGGTTGTNCGACCCTTGATTCNAAACNNTTNGGNNNAGGGGGNANNNNACG
ANANGNAANNCCNACTNNNNANTTTNATGNTNAANTNCCAAACNCCNANTCNNAAACCCANAATCTT
CTNGCNGGGAANTCGGNGGGCCNAATGAANGGNGNNCCCCTTNACCGNGAANGGTTAACAGACTTAT
ATCCAANCCCCTTCGNGGTGTGNGGAGAGCAAANANNGANATGTGNTGACACNCCCACGTGNTATCG
CGCGCNCATATATGTGCACGANCACGANNCCATGTGAGAAANTTTGGTTTTNGAAAAGCGANNGAGA
CNCNGTNGCTCTTATNAAAGGGGGGCACNCNCNNNTGTGNTTTTTACACAAAAANTCTCACTCTACNN
CCTTTTTATAATATGGCCCCCNCACCTGGTTGNNAACCTACTANGGTCAANNGTNTTTNNTANTGGTG
CGGCCNACCTTCTTCGNCNTTATACTANCTTNNGACCTNTTGTTGAGACCCCCNAAATNGGTTNATTCC
TNGGNGAANAAAGCANNCCTNCTGNTAATTATTCCTNCGAAGGATGGGTNCNTCTTGTTANNCCCANC
CAAAAAAAATTGGGGTGGGNTTTCTTGGGACCNCCACNNANGGAAANAAATGANTTNCNTTNNTGA
CCTTCTTTTNTTCTTNNTGCCTNCCGGAAGCTTNATTAACTTTTTTTNTGGGGNACGNAGNTTAATCAA
CCNTTNNNNGNGCNCNGATTCNNTTTATGGAGGGAAAAAATTCNTTNNNTAAACCCCTNCCCNNNCNN
TNTGTTTTGCACAAAATNNTTNTGCNGGCGGTNTTTNCCTTTNNNNCGGGCCTTN
TTNANNAAAGCTGGAGCTCCACCGCGGTGGCGGCCGCTCTAGAACTAGTGGATCCCCCGGGCTGCAGG
ATTTTAATTGACATCTCACAGAGGTCCAGAGACATANAAAGACTTAACCAAAGTTAGGGTTGGAATTTA
GAGAAGCCAAATACCCTTTTTAATCAAGATGATTCTGAATATTTTTATGCTGATAAGGAANCAGTAAA
AGTGATTATTAATTTTTTTTCTCTTAAAGTTTGGATCCTGGATGCAGTTGGAGGACAAGAAAACCCTGC
TCTTTCACTGTGATCACNGTNGNNTACAAATGATTGTAGGTGATAAAATTTCNGATGTTGGGTTNCCTC
CACACANACTTTNCCAGACTGTCCTTNACATTATAAGACGACTTCTTTCCAACTTCCTTACATTTGGGA
ACTTTAGATTNATAGGGTATTGGGTNCTTAGTCTTAAAAGCCTGGAGAACNATAAAAGAAATTCCTTA
AAATTTTAACCTGGTTTCCTAAGTAACAGGANCGGAGTTTCCTTTGGAAACAAGAAAGGCCNATNCCANT
AGATGAAGNGTTTTTTTACAGGTNTAATAAAAAGTTNCCATGGTTAAAGGGAACCAAAACCGGACCG
ATAAGANAANAGTTTTAANATTTGGCCGATAAGAATTTGGAAAAATGAATGGCCCNTTGAAGAAGAN
AATGGTTCACCTTGGNAAGGATGACCTTGGNTTTGGTTGGCANCNTGGAAAAAAAAAGCAAANGGAN
CTTAGGGNTGACCTCAAAANATGAAGGATTCCNAAATATGACCANCCTTGNTAANTTGGGAAGNAGG
ANGAAANTTTCCCTTAGGAAANNTTGAAAGGAAAAATTTNCCTTNAAATTTGAAGGAAANAAGGGGG
GAACCNTTNNNGGGGGGGGGNNNNNNNGANNNAAAAAANGNNGGGCCTTTTCCCNAAANNTNNNN
NGGGGGGGG
TTNAAAAANCTGGACTCCCCGCGGTGGCGGCCGCTCTAGAACTAGTGGATCCCCCGGGCTGCAGGAAT
TCGGCACGAGGAGGAGAAGGGCACCACTGAGGAGGTCTGCCGCATCTACCTGAGGCGCATTCTCCAC
ACCTACTACAAGTTTGATTACAAGGCCCATCAGCGGCAGCTTACGCCCCCTGAGGGCTCCTCAAAGTC
TGAGCAAGATCAGGCCGAAAATGAGGGTGAGGACTCAGCTGTGCTGATGGAGAGACTGTGCAAGTAC
ATCTATGCCAAGGACCGCACAGACCGAATCCGCACGTGTGCCATCCTCTGCCATATCTACCATCACGC
CCTGCACTCCCGCTGGTACCAGGCCCGGGACCTCATGCTCATGAGCCACTTGCAAGACAACATTCAGC
ATGCANACCCACCAGTCCAGATCCTGTACAACCGCACCATGGTGCAGCTGGGCATCTGTGCCTTCCGC
GAAGGCCTGACCAAGGACGCGCACAACGCCCTGNTGGACATCCAGTCAAGCGGCCGGGCCAAGGAGC
TTTTGGGCCANGGTCTGCTGNTGCGCAACCTGCAGGAGCGCAACCAGGANCAGGAAAAGGTGGAGCG
GCCCCGGCAGGTGCCCTTCCACCTGCACATAAATCTGGAACTGGTTGGAATGNGTTTACCTGGNGTNA
GCCATTGNTCTGGGGANATCCCTTACTTGGNCCCCCCACAAAAGTGACCCCCCCCNACCCATGATCAA
GCAAGCCANTTNCATTACCCANTTGNGGGTGGGCCAAACCGGAAACCCNTTTNTTGGNCCCCCCTTGA
TTCCATGAAGGGAACATGGGGGTTGGTTGCCTTTTAAGGCCCTTTAAAAATGGGCCNAATNGGAAAAA
CCTTGCCCCAAATTTTTTTTTNTTTCATTGGGAAAAAAAAAAAATGGGGCAAAAAGGGNGGGGAACCNT
TTTTCCCGGGNG
NACAAGCTGGAGCTCCACCGCGGTGGCGGCCGCTCTAGAACTAGTGGATCGCCCGGGCTGCAGGCTG
GTGATTCTTTAATGATTTGAAGTCCATTGCTTGGGAGGAAATTCCACCACACATTCATGCTTATAATAAG
CTGGGGATTTTTGTTTGTTTTTGCAAATGCTTGCCCCTACTTTGCAACGATTTTCTGTTCGTAATTCTGA
AGAACCAAGGTGGGGAGCAACACAACTGATGGAATAGTGGTATAAGTTTATACCAGAAGCTTAATGA
CAAGTTTTATCAATCAGAATAATACTTGGTTATGGAAGTGACTGATGCTAAATAAATTCTGAGTATTTT
TTATTAGATAATTTCTCACATATAGACTTAAACTGTCAGCTTGGTAGTGTCTATTAGTTAAACTTTGTA
AAATATATATATATTCTTGTTTTTCCATTGTATGCAAATTGAAAGAAAAAGATGTACCATTTCTCTGTT
GTATGTTGGATTATGTAGGAAATGTTTGTGAACAATTCAAAAAAAAAGATGAAAAAAATTCCTGNGGA
TGTTTTGNGTAGTATCCTTGCCATTTGTATTAATAGTTAAAGTTCACTTCCAAATAAAACTCCCATAAT
GCTAAAAAAAAAAAAAAAAAAAACTNNNGGGGGGGCCCGGNCCCCATTTCNCCNTATAGGGGGGGN
NNNNNNNNAAANNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
TTNNACAAAGCTGGAGCTCCACCGCGGTGGCGGCCGCTCTAGAACTAGTGGATCCCCCGGGCTGCAGG
AATTCGGCACGAGGATCCCCCATCCATGAAAGCTAAGCCAAGGCCACCNGCCTTCATGATGCACNACN
CGGTGNGAGTCAAGCCCGNGCATCCACGGCTACTCACTTCGCCNNAGCCTCACGTATGANAGCTGTGG
NCAACATCTTCACAAAACATAGACCAAGANGCATTACAGAGGCTCTTCACAAACTCCGGGGACANGA
AAGCAGAGGAGCGAGCAAAGATCATCTTTGCCATANATCAAGACCTANAGGAGAAGACACGAGCCCT
GATGGCTCTGANGAAGAGANCGAANGACAAGCTTTTCCAGTTTCTGAAACTGNGGAAATATTCCATCA
AAGTTCACTGAGGACCAGAGGATGGATAAGGACATTATCCAAGAACGGACATTTAAAGACCAAGTGA
GTCTGTAAGACCCTGACACGCACAGCATCTTGCTGTTGCCTCGTACGTTTCTTCTGTCAGGACTCCG
GGACTCGGGGCAGGAGAGGAACAGAATGAACTGGTGACAAGGACTCCAACCAATTTCATTGCCTGTG
CTGGTCCAGTGGAGAAGTGCTTTCNGCAAGGACTGGAAAACTCTTCCTGCANGAGAAAGACTGGTGCT
NATGTCANGAAGGANAATTCTGAACCGGNCCTTANTGGANAAGATGACCNATGGCTTGANAAAGGAC
TTGCCCCCAAGGTCTTCCCGGGCCNGGNCCTTGGGCTGGAAATGGACCCNGGAACTTTTTTTTNCCTTA
AACCCAAATTGGNAAANNTNAATNGGGGATGAACNTTTCAAGGGGGGAATAAAAAAAAAAAAAATT
NGGTGGGGGGNANTGGGCTTTACTTGGGGNTTTTTTTTTTTTGNAACCCGGGNCCNGN
NNCANANCTGGAGCTCCACCGCGGTGGCGGCCGCTCTAGAACTAGTGGATCCCCCGGGCTGCAGGCTC
AACCAGTCTTAAAAATGACACTGATTCATAAACGATCATGACCATTCCATTTATCCTTACTATTTAGAA
TACACTATATTGCCTTTTTACTCTCAACACAGGACGTATTGACACATGTTGGAATTTTCAAGATGCCTT
GTTCATCAGTAAATAATCTGACACAGACTTGAAATAAGCTCAGTATATAAGTACCCAGTTCTTACCTGC
TTGCCTTAAAGGGCTCTCGGTCGAATGGCCTTGTATTTAGAGATCTAAGTGACACCTCCTCATGTTCTC
ATGTAGTAGGTACGAAATCAACTTCAGTGACAGCTCAGCAGTGCTGGTTATAATGATCCTATGATGAA
TTTTATCAAGCCTAATTGCGAGAGCTGCTATTTAGCAAATATTTAAATTGCTCAGACTGAGGGAAATA
ATTTTCTGGAGTCTTCAAAAATAAATTACATTCATTAAAAAAAAAATACATTCATCAACAACAACCAC
CACTATACTTTAAGTAACTTGACTGGTAGAGCCAACCCTGATATGCATGAACTTTTCATTGGGNGGGA
GGCCATAAAGCCACTTGTTTCAAAAGAAGAGTCCTATGAAGACACGATAGCACTATTTTTNCTGAAAA
TGCAGGGGTCNCTTCCTAGCTGGTGGAGCCTGGCGATGGCATGTGTAAAGCAAAAACCCTGTGCCCCT
CAAGGNGGGGGGGCNCAAAAAAAAAGGGAATGGGGAACCTTGGTGGTTTCANNAAAGNNNANGGGA
TGGNTTTTCCCTTTTGGGNGGGGNCNAAATTTNAAANCNCCTGGNTTGCCNNCCTGGGGGANGGGACC
CCCCNNTTNNCCTTGTTTTTTTNNCNNCCCCGTTTNCTTTCNGGGGNNGGGAAAAAAAAAAANGGANA
NNNNGNTNTGGTTTTTNNGAAAA
GCCCTATNTGGAGCTNCACCNCNGTGGCGGCCGCTCTAGAACTAGTGGATCCCCCGGGCTGCAGGAAT
TCGGCACGAGGGAANAACTANCTCAGTATCAAAAGTGGGATAAGATGAGACGAGCCCTGGAATATAC
CATTTACAATCAGGAACTTAACGAGACTCGTGCTAAACTTTGATGAGCTTTCTGCTAAGCGAGAGACAA
GTGGAGAAAAATCCATACAATTAAGAGATGCTCAGCAGGATGCAAGAGATAAAATGGAGGATATTGA
GCGCCAAGTTAGAGAACTGAAAACAAAAATTTCAGCTATGAAGGAAGAAAAGGAACAGCTCAGTGCT
GAAAGACAGGAACAAATTAAGCAGAGAACTAAATTGGAGCTTAAAGCCAAGGATTTACAAGATGAAT
TGGCAGGCAATAGTGAACAGAGGAAACGCTTTATTAAAAGAAAGGCAGAAGCTGCTTGAAAAAATAGA
AGAAAAGCAGAAAGAACTGGCNGAAACNGAACCTAAATTCAACAGTGTAAAAGAAAAAGAAGAGCG
AGGAATTGCTAGATTGGCCCAAGCTACCCAAGAGAGAACGGACCTTTTATGCAAAGCACGGGTCGAGG
AAGCCAGTTTACATNNNNAAAGAAGAAAGGGATAAGTGGATTTAAAAAGGAACCTCAAGTTCTTTAN
AATCAAGCCTATTTATTTGACAAAGAAAAAGAACAGATTTGCTGNTTTTACCATAANGGATTTTGGGA
GGGACCACTTGGGGGCCNANATTANAAGAGAAAAAATCTTTGGGNGCAGTNNTAAAAANACCTGGGAN
TTCNGGGATNTTTATATGGAAATTCCAAAGNCTCNGGGTTTTGGAAGAACCTNGGACANTAAAATTTNC
TTNTTGNAGTTTNAATAANTTNTTNATAGANNAATTTNCCAANGTGGGNTAATAAATTTTTNCTTTGTGG
NNGGAGGNAAAANGAATNGCAGNAAACTNCCNCGGGCCCTTTGTNN
GGGNGGGGGATGNGAATNCTTNTTTNTNNANTTTNTNTTNNNNNNNNCCCCTNCCTNTTNCGTTNNTT
NNTNGNTGNNCCTACTCNTATTCNNTNTAAGGGGNGGAGGGTGGCGAGNCNNTNNTCTTTGGANCNCC
NTTGGAGACNTNCCGTTTTTTNNACANACNTGACACANGGNCATNCNTNTCTTCNANNAACNTTNTNG
TCANCNAANNAAATGTNANCAACACANGNTNGGATTCNTNATTNNTACCNNCTCGGGGGTNNNATTTT
TATNNAGAGCGNGTGNTTNCTCGNTNCTTACGTTNNAGNNATACTGNATTGNNNNTATTATGTGTCTA
NAGGGTTANTATCGCNNAGGGATNNANCATTNNNNTNTNNGTGANNCGGTNGANTGATGNAANGTGA
NGGTNNAATNTNTANNANANAGTTCTGANTNGTGTTNNNNNCCCTNANNNCNAAGANTNTGTGNNGN
NTNNNANGATGTAGNGCNTNGNGNGTGGNGCNNNTTTGATCCACTGGNGNGNNCNNGGAGANNTNNT
TAGNAGATCNNNNTTNTNCCATNACAGTATANNAACTNNNNGGNAGGGNNGAGNNNGNGATCAGNC
GANANGTNNNNNTGTAGCNCGNAGNCTCTGCNACNNANNCNTGATGNTNTNNNNTGANAGTTGNTTG
GCNNTNNANGNNNNNGGGNAGGAGNNNGTNTGTNTCGATACNNNANCNCNGTGAGATTNCGGNANT
NGNNACAGNCACTAGNGNTNTANATNNAAGATATAANANNAGNGNNATGNNNNTCTTGAGNTNNAG
ANNNGCGNGGAGTGNGNTTNGNATNTGNNANTNNANTCNGNTNNGNTNGTNTNNNTNNAGNNTNTNCNA
NATTNNNTTNNGGGAGTCNTNGTNTCGTNNTNANGTTGCATTGATANGNGTNTAANANANNATANTNT
GTCANGANTNANTGGAGTTNACNAANNTGNANTNNNNTGGGANACNTCATNAGNTCTNCTCCCTGTA
NNCTANANTCGCTNTCGANGGGANNNTCNNGGTAGTCTCCCGNNGTNAGCCNTAGNCGTAGTCTCNN
GTGGNNNTAGATAGTATNANTNNGCATNNTNANTNNTNANNNGNNGACAATGTGATNNGANGNTTGN
TGTACGANNNNTGANGTGNNTGCATNNCTGAGGNNGGGTTTNGNNTACNCATGCNTNNCGNGGAATN
TANCNCAGNCNANTANGGNANNATNTANTGATAATCNNACCNGTACNNATCNNTCGNTATGNGCNCA
GGGCACNTNTATCGTGNGNGNGNTATCGGTANTNNAGNCGGTNGNGGATNTNTGTGTGTACTNNGCN
CGCATNNNACGAGGTGTGGNGAANGTGANTCNCTGTGAGGNGTATAGACGGTCNTANTGANAGGAGA
CCNAGNACGCNGATGTNTGATGNTANCAANTNNGACNTNNGANGTATNCNACGCGGATNNNGATGTN
ANGNNTCGGTGANNTTTTTCNNTCTNNNATNGCNCNNANNTNANAANGGNNACANACACNGGNNGTG
ANNNANGNNNNTTGTAGATCNCTGTAACGTNNCGATNTCGAGTTGNGGNTAGGANTCNCTGNAGTGT
GTGTNNACGCG
GGGNGGGGGATGNGAATNCTTNTTTNTNNANTTTNTNTTNNNNNNNNCCCCTNCCTNTTNCGTTNNTT
NNTNGNTGNNCCTACTCNTATTCNNTNTAAGGGGNGGAGGGTGGCGAGNCNNTNNTCTTGGANCNCC
NTTGGAGACNTNCCGTTTTTTNNACANACNTGACACANGGNCATNCNTNTCTTCNANNAACNTTNTNG
TCANCNAANNAAATGTNANCAACACANGNTNGGATTCNTNATTNNTACCNNCTCGGGGGTNNNATTTT
TATNNAGAGCGNGTGNTTNCTCGNTNCTTACGTTNNAGNNATACTGNATTGNNNNTATTATGTGTCTA
NAGGGTTANTATCGCNNAGGGATNNANCATTNNNNTNTNNGTGANNCGGTNGANTGATGNAANGTGA
NGGTNNAATNTNTANNANANAGTTCTGANTNGTGTTNNNNNCCCTNANNNCNAAGANTNTGTGNNGN
NTNNNANGATGTAGNGCNTNGNGNGTGGNGCNNNTTTGATCCACTGGNGNGNNCNNGGAGANNTNNT
TAGNAGATCNNNNTTNTNCCATNACAGTATANNAACTNNNNGGNAGGGNNGAGNNNGNGATCAGNC
GANANGTNNNNNTGTAGCNCGNAGNCTCTGCNACNNANNCNTGATGNTNTNNNNTGANAGTTGNTTG
GCNNTNNANGNNNNNGGGNAGGAGNNNGTNTGTNTCGATACNNNANGNCNGTGAGATTNCGGNANT
NGNNACAGNCACTAGNGNTNTANATNNAAGATATAANANNAGNGNNATGNNNNTCTTGAGNTNNAG
ANNNGCGNGGAGTGNGNTTNGNATNTGNNANTNNANTCNGNTNNGNTNGTNNNTNNAGNNTNTNCNA
NATTTNNNTTNNGGGAGTCNTNGTNTCCTNNTNANGTTGCATTGATANGNGTNTAANANANNATANTNT
GTCANGANTNANTGGAGTTNACNAANNTGNANTNNNNTGGGANACNTCATNAGNTCTNCTCCCTGTA
NNCTANANTCGCTNTCGANGGGANNNTCNNGGTAGTCTCCCGNNGTNAGCCNTAGNCGTAGTCTCNN
GTGGNNNTAGATAGTATNANTNNGCATNNTNANTNNTNANNNGNNGACAATGTGATNNGANGNTTGN
TGTACGANNNNTGANGTGNNTGCATNNCTGAGGNNGGGTTTNGNNTACNCATGCNTNNCGNGGAATN
TANCNCAGNCNANTANGGNANNATNTANTGATAATCNNAGCNGTACNNATCNNTCGNTATGNGCNCA
GGGCACNTNTATCGTGNGNGNGNTATCGGTANTNNAGNCGGTNGNGGATNTNTGTGTGTACTNNGCN
CGCATNNNACGAGGTGTGGNGAANGTGANTCNCTGTGAGGNGTATAGACGGTCNTANTGANAGGAGA
CCNAGNACGCNGATGTNTGATGNTANCAANTNNGACNTNNGANGTATNCNACGCGGATNNNGATGTN
ANGNNTCGGTGANNTTTTTCNNTCTNNNATNGCNCNNANNTNANAANGGNNACANACACNGGNNGTG
ANNNANGNNNNTTGTAGATCNCTGTAACGTNNCGATNTCGAGTTGNGGNTAGGANTCNCTGNAGTGT
GTGTNNACGCG
TTNANNAAAAGCTGGAGCTCCACCGCGGTGGCGGCCGCTCTAGAACTAGTGGATCCCCCGGGCTGCAG
GCAACAACTGGAAAGGAGCGATGATGTTTAATTTGTTTTGTCCCGGCAAAAGTACAATGTGCTGCAGTT
TATGACAGATAATTTAACAAAATATATATATATATATATATATATAAATTGTATATAAAGTCTGTCTGA
AACATTTCTTTGGGAGGTGTTTTTTTTTTTTGGGGGGGGGGGNANNCTTTNNAAAAACCNTNNAACNTTT
TTTTNNTNNGGGGTTTNNTTTNCNTNNANNAAANACNTTTTTNNNNGNAAAAGGGGGNCCNCCCNTNT
NAAAANGGGTTNGNCNTTTTTTTGNAAANTTTTNNAAAAAANGGNANTNGNNCTTTTNATTTAAAGGG
GGCCCCCCNNGAAAANNTTTTTNTTNNNCCNCNAAAAAAACNTTNGNNNTTTNGGCNNNNAAAAANN
NNTTNNTNAANTTCCNAGGGCCCCNTTNNAAANNCCCCCCCNNGGNTTGGGGGGGNCCCCCCCGGNN
TTTTNAANNCCGGGAAAAAAACNNGCCCCCAAANNTGGGTTTNCTTAAGGGGGGNANTTTGGGGAAC
CAATTTTTATTAAAGGGNCCCCCCAGNTATTTTTTTTTTTAANCCGNCCCCCAANANAAATTTNCTTTC
CCTTTTTTAACCTTTGGNTTTNGNCCCNAAAACCCAAAGNCCCCNTNTGGGAAAAACCNGGGTNATNT
AAAGACCAACCCGGGNTTGGGNTAATNAAAAAAAAAAGGGGGGGTTTCCTTTTTTTTCCNGGGGGNNN
AAAACCCCCCCGGCCTTTTTTTTNGCCCCCCCTTTTTANCCCNCCNTTTTTTTNAAAATTTGGTTTAAAA
AACCCCGGAAAAATTTTTTTTTCCCCCCTTAAAAAAAAAAAAAGGGGCNNGGAAAAAANTTTTTTCCN
TTTNTTTTT
TTGAAAAAGCTGGAGCTCCCCGCGGTGGCGGCCGCTCTAGAACTAGTGGATCCCCGGGCTGCAGGGGG
AGGAGCAGAGGTAGNGGATGAGATANAGCTGGTGTGTCAGCGCCGGGCCTTGGAGGCCTTTGACGTG
GATCCTGCTCAGTGGGGAGTCAATGNCCAACCCTACTCCGGATCCCCAGCCAATCTGGCTGCCTACAC
AGNCCTTCTGCAGCCTCACGACCGANTNATGGGGCTGGACTTGCCCGANGGGGGCCATCTCACCCACG
GCTACNTGTCTGATGTNAANCGGATCTCANCTACATCTATCTTCTTCNAGTCTATGCCTTATANGCTGA
ACCCCCAAACCGGCCTNATNGACTATNACNAGNTGGCACTCACTGCTCGGNTCTTCAAACCACGACTN
ATCNTAGCTGGACCAGGGCCTATGCTCGNNTNATTGACTACGCCCGCATGANAGAAGTGTGTGACNAG
GCCAAGGCACACCTNTTGGCAGACATGGCCCACATCANNGGCCTGGGGGCTTTGCCAAGGGGATCCCCT
AACCCTTTAAGNACGCGGATATTGTCACCACGACCACTCACANGACCNTTNAAGGGGCCAGGTCAGG
ACTCATCTTTNTACCGGAAGGGGTGCAGGCTTGNGNACCCCAAAACTGGCCGAAANAACCCTTAACCCN
TTTGANGGACCGAATCACNTTTGCTGGGTCCCCTTCCCTGGAGGGGGGCCCCCAAAACCTTGCCATTG
GTGGAAGGGGCTGGGGNCCTNAAGCAGGCCTGGNNCCCCCCTTTTCCGGGGAAGACCTCCCTGGAAA
ATTTNAAAAAACCCTTCAGGCCCATGGGTTAAAGGCCCTTGTTGGGACCGAAGGCTACTCCCTGGGGG
GTTNGGGGGGCCCTGGCCAACCCCCCTANGGGGNGGGGGNAACCCTTTGGGCCNAAAGGGCCG
TNNNNNAAAAGCTGGAGCTCCACCGCGGTGGCGGCCGCTCTAGAACTAGTGGATCCCCCGGGCTGCA
GGTTGGGTCCTTTTCGGGACCCCCACCCCTCCAGGCCCCTCTTTGCACACTTCTTCCCCCCTCCTGCCCA
TCTTTCCCCGCACTGCCGTGCCAGGCCTGGAGGTGGTGGGGTGGGTAGAGGCTCAGCCATTACCATTTC
ACACCTGTCCCAGAATCTGATACCCTGCAGAGGGCAGNGACCACGCTGTTCTCCGAATGAGAGNCTTT
CCCACAAAGACCTGGTATTGGGAACATCCCTCTCCTTTGCCTCCACTTTCCCAAGGTAAACCCCATCCTG
AAATCTCTAGCCTGGGGAGGCAGAGACCCTGCAATCCAGAGAACACAGCCTCAGAGGTGCTGGAGGA
GGGACCCTGGGGCCTGCATAGGCCACTGTGGCCTCTGCTGTCCTGCTGGGAGGCAGCCGTGTCTCCCC
AGTGGGGCTCGTCCCTTCGTCACTCACACACTTGGGCCTGCTCCACTGACATCACAAGTCTTCTCCACG
TGATGGTTTAAAAGGAATCGTTTTCAACAGCAAAGAAATGGAGGTGGAGACAGCGCTACCCTGAGTGA
CACAAGGAACGGCCCGNTGGCTGACCCACGTCCGGGCCTGCCCTTGTTGNCGTGTGGCGGCTTTAGAG
TCCGATGGGGCANGGANGGCNCANTGTNCCANCACANGGGTCTGGAAGCCGTTTTCAANCCTTTTGNT
TNCCGGAAGGTTCTTCTTNCCATCCGGGTAACNANTTCTGTCCTTTGTCCCGTCCCCCAAAAGCCTTTG
NAGGNTTNTTTTTTTTTCCAAATGNGGAANCTTATTTTACAATTGAATAANAAACNTAAGGNCCNAGG
AAAAAAAAAAAAAAAAAAAATTNGGGGGGGGGGNCCGGGNNCCAAATTTCCCTTTTTNT
TNNNNAANACTGGAGCTCCCCGCGGTGGCGGCCGCTCTAGAACTAGTGGATCCCCCGGGCTGCAGGA
ATTCGGCACGAGGCGACAGATAGCCGTAACTTCTGTGCTGTGCCAGCCGCATCCCTGAGACAAGATGG
TGAAGGTCGGAGTGAACGGATTCGGCCGCATCGGGCGCCTGGTCACCAGGGCTGCTTTTAATTCTGGC
AAAGTGGACATCGTCGCCATCAATGACCCCTTTCATTGACCTTCACTACATGGTCTACATGTTCCAGTAT
GATTCCACCCACGGCAAGTTCAACGGCACAGTCAAGGCAGAGAACGGGAAGCTCGTCATCAATGGAA
AGGCCATCACCATCTTCCAGGAGCGAGATCCTGCCAACATCAAGTGGGGTGATGCTGGTGCTGAGTAT
GTGGTGGAGTCCACTGGGGTCTTCACTACCATGGAGAAGGCTGGGGCTCACTTGAAGGGTGGCGCCAA
GAGGGTCATCATCTCTGCACCTTCTGCCGATGCCCCCATGTTTGTGATGGGCGTGAACCACGAGAAGT
ATAACAACACCCTCNAGATTGTCAGCAATGCCTCCTGCACCACCAACTGCTTGGCCCCCCTGGCCAAG
GTCATCCATGACCACTTTGGCATCGTGGAGGGACTTTATGACCACTGTCCACGCCATCAGTGNCACCCA
AAAAACTGNGGATGGCCCCTCCGGGAAGCTGTGGCGTGACNGCCGAAGGGCTTGCCCAAAATATNAT
CCCTTGCTTNTACTGGCCCTTGCCAAGGNCCGNGGGCAANGNCCNTNCCTTGACTTAAANGGGAAACT
TAANTGGCATGGGCCTTTCCGGGTNCCCAATTNCCAAANNGGTTGGTTGGGGGATTTTGACCCGGCCC
NCCTGGGNAAAACCCTGCCCAAGTTTTAA
CCTATCTGGAGCTCCACCGCNGTGGCGGCCGCTCTAGAACTAGTGGATCCCCCGGGCTGCAGGAATTC
GGCACGAGGGTGAAAATTAAAGCTAATACTGCTTAAGTGCTTATTATGTGCCAGGCACTGTCCTTTAT
AAAATTTGACTCATTGAGTGAAAAATATATTATTTCTAAGTGTTGTGTGTGTTCTCTGAGGAGGAGGAG
GAACAGAGAGAAGGTAAGGCTGGAATCAAAGGTTGGTGCTTCAAGAAAATGACTTCTAAATCGGTGT
TTTTCTCATCTTAATTGGTGACTGTATTCCTGCCATCTATACATTAGAGAGACTGAGTTAACATGACTTC
CTGTTCACTGTGGGTTCATGTCGTCAGCAGAGAGCATGCAGTTTGGGCTCCGACTGTACACCTGGGGTT
ACTGGTCTCGGGGAAGTGGTGTGAAACAGAATTCATACCAGGGAGCCCTATAATAACCATATCGGGCA
CTTAACCACATGCCAGGCATCCTAGTGAGTGCTTCACATGCGATACTTTTGTTCAACCTTTTGACTGCCC
TTTGAGAATGGGAATCTTCCAGCCCCAGGCTCAGGTGAGGAAAAGGAACTCAGTGACACGACCAAGG
ACTTGCTCCTCATAAGGTGGGGAGAGGACCCCTTAGCCCCCAGGACACTGATGGACTTGCCTTTCCCCT
TCCCAGACTGCTTTCTTCGGAGGAAGTGTCGCTCTGTGATCCAGGAGCAGGCTGCAGCCCTGGGCCTG
GNCATGTTTTCTTTTCTTGGNGCCGACGCTTCACCTACTTACTGGAANGAGTCGGCCAAAGCTTNACTT
GTCCTNTCCTGAGGGGAGANGGAGGATCAANGATGACCTTCAAGGNGTCCTNCTTTTGTCCCGGANCC
TGNAAGGAACTGCTCCCCCANNGNGAANGNNTNGCCAAAACTGGATGCTCGGGCTAGCCAAACCCTG
GAACCCTCCCCTACCTTCCTGGGACTGCCCTACANGG
TTGAGNCNATNTGGAGCTNCANCNCAGTGGCGGCCGCTCTAGAACTAGTGGATCCCCCGGGCTGCAGG
AATTCNGCACGAGGATGNCNNCTACGNNNCCCAAGGANCTTGTCTNGCTGTGGGNCTGAATAACANN
TTATTCCTGCANAAAGCGGGCANGCTTGACTNCANTCNCAAGATAACACGNGCGCNCACCTGNCTGA
TNTNTGANACCGACCTCTGANTTAAGACTGNCGCCACANCNACNTGGNTTNCNTGGCNNCTCCNCCTN
GCTCCTCAANANNANCCNTTCCTGNCAANATCCTNGTANTTNGAATTCAACTCNAAANGGNATTGNTN
GACNCCNTTTNCNATCTCAGACTTTTGNNTTTNAATGGCNNNNTATCCACTGANNATANTATTNCCCTA
CCTCNCNTTAACNTGAATATATATTNTCNCTGTCNNGNTATTNTTCTCCTCNCTGATCTAAAANACNAT
NCNTNCCGATGCNTAAATGTCNNTCCTCATGCTNNNANACCTNGNTTTNATNGGAACNAATNTTTNTN
GTNATNTCCNNAGNNTTNCNNNNNTCACTTNTNAATNGAANNCTNTACTNTNNNTTNTNTTTCNTTAATA
GGGAANNAANGCATGNNNNTTTAAACTTAATTCCGGNNANNCNTTCCNNNNAATCACCTAATCTNGA
ANTGNNACAAGTACNCTAGAANGGTNGGGCANAATTTTANATNCCCAANACANTTGGNGCTCTAACGA
CNNCANGTCAAGGNNCATTANNCNCNCTAAATGNACTTACTNNGNCCAANGAGTCNNNNNNNCANCT
NTAGNGNATNACAAGATNNTNGNNCCNGTGNNNCNTTNCNNANCNTGNGNNTNTNNTGGGTCATNCA
ANACTCNANGCGTCTCTTCTACCTCTNGCTNTTCCCTTCNNNATCTGNTNCANNCANTNATCCCTANTN
CNNNGNTNNTCTTTTTTTTTGNNANTAAACCCGGAACTNTCTAGTTNCACCTCNCCNNTTNCNAANTAA
ANNTTTCANTAACTCTCNTTTCANCTCANNNGNNCTTTNNGCNACNTCNTGTCNACNCNTATTATTCCC
NNTNCNCNAGTTTTCNNTCCN
GCCCTATANNGAGNCGNATNACAGTGGCGGCCGCTCTAGAACTAGTGGATCCCCCGGGCTGCAGGAA
TTCGGCACGAGGGTCATGTTGAGCCTGGAGTTTGATTACATGTGCCAATATGACTATGTTGAGGTCCGT
GACGGGGACAGCAGCGACAGCCAGATCATCAAGCGTTTCTGTGGCAATGAGCGGCCAGCTCCCATCC
GAAGCACGGGCTCCTCACTCCACATCCTCTTCCAGTCAGATGGCTCCAAGAACTTCGATGGCTTCCATG
CCATCTTTGAGGAGATCACAGCGNGTTCCTCGNCCCCTTGTTTCCACGATGGCACTTGCCTNCTTGACA
GCACCGGATCTTACAAGTGTGCCTGCCTGGCGGGCTACACCGGGAAGCACTGTGAAAATCTNCTTGAA
GAAAGAAACTGCTCANACCCTGGGGGGCCAGTCAATGGGTACAAGAAAATTACAGGAGGCCCTGGGC
TTATCCATGGGCACTATGCAAAAATCGGCACCGNCCTGACCTTCTTTTGTAACAGCTCCTATGTTCTCA
GTGGTAATGAGATGAGAACTTGCCACCATAATGNANAGTGGTCNNGGAAACAAGNCCNATCTNGNAT
AAAAGCCTGCCGNGNAACCAAAGAATCTNCAGACCTGGGTGGAGACNGAAAGTTCTTTCCAANTGCC
AGGTTCACTCCAAGGGGAGAACNNCCNTTTACAATCAGNNTNTTACTCATTNAGCCCTTCTACNANAN
CANNAAAACTGCNNGGATGCCCNCNTACCCAAAGAAANGCCANTNTTCTTTTCCCTTTTGGGAAGAAC
CCGGCCCGCNCTNGNGTTCCCAAACATNTTGCNNCACCCCCNNTTANTCCANGTACCNAGGTGCTNTC
TATCACCGCTTTTTTTATTCNGGCCGACCTTGGGGCANCTAANCNCGCGNANGGACCATTNTTTTTANG
GGAACTNGGTNAANATGGGANANGCGTCCNTTNNCCCCCATACCC
TNAGNCCTATAGTGNNGTCGTATTACAGTGGCGGNCCGCTCTANAACTAGTGGATCCCCCGGCCTGCA
GGAATTCGGCACGAGGATGGCACGTCCCCAGTGGTGGGGATGTGCCGCGCTTCCTGANTAANTGGAAT
TGGNAGNAGTGNCTGCCCNNTNNNGNNTTTNNTNGGNAAAAAGGTNNCNCCNNTTNNNNATNAANNNT
TTNCNNGNNNNNAAANNNNNNGGGNNGGATTTNNNNGNNGNTTTCCAAGGGNAGGNNCCTTTTTTTN
NGGANNTTTGNACNCTTNGNAGAANNCAGCCTGTGTANTTAACCGGTGGGAAGNCTTGNGNGTTGNG
CATTCGTNCTTGNGCACTATATTTGCGCCTGCGCTATGNGCACAANAGAGCCTGTGNCTGTTNAGGCG
CCTGTNTTTTCANACCCANNTNTGNGACANCAAGTGCNCNACANGNTNTCAGGGNTNGGNGTGTNCA
NGANNCCCCTCTATATATGNNTTTGCGNGCGNCNTGGANNACCCACACCCCTATAGCNGGNGTGATAN
GCGCTGCTNNTANCAGANNAGGGNGCCNCCAGTGTAANNTCTTTGAAGGNGANCCCNCCTCCNNTCTA
ACNTTCNCCTNNTGTAGAGNGCGCGGTGGAGATTCCNAAAGAGGCANAAANNCNTGNNGCTNGGTTN
GGANAATCNTCTGNTTNCNNTCGNTNCTTATTNANGTGNCCNTNNNGCCCTCNTTNAATCANANNATN
NTNTNNTTAACNNNCTTTAACCANCTNNGTTATCTTNGNGTTCCTGGTTNANTTNTCTATAAAANTCNT
NNTNCNTAGNGAAANAAAAATGNGNNAGAAACNACCGGTNNAGNNCNGCCTTNTTTTCTNTTNCTTN
NGCNNAAGTANCTNNCCTTCTTTNNGGGACGGAANNANCCTNTTCTTTCCTCCTNNTTTTTNCTANNGN
TAAGTCTATATTAAAGCCCNCNNNCCTTCCTANACCNNNTCCATGTCGCCAANATNACACAACCG
TNAGNCCTATAGTGNNGTCGTATTACAGTGGCGGNCCGCTCTANAACTAGTGGATCCCCCGGCCTGCA
GGAATTCGGCACGAGGATGGCACGTCCCCAGTGGTGGGGATGTGCCGCGCTTCCTGANTAANTGGAAT
TGGNAGNAGTGNCTGCCCNNTNNNGNNTNNTNGGNAAAAAGGTNNCNCCNNTTNTTNNNATNAANNNT
TTNCNNGNNNNNAAANNNNNNGGGNNGGATTTNNNNGNNGNTTTCCAAGGGNAGGNNCCTTTTTTTN
NGGANNTTTGNACNCTTNGNAGAANNCAGCCTGTGTANTTAACCGGTGGGAAGNCTTGNGNGTTGNG
CATTCGTNCTTGNGCACTATATTTGCGCCTGCGCTATGNGCACAANAGAGCCTGTGNCTGTTNAGGCG
CCTGTNTTTTCANACCCANNTNTGNGACANCAAGTGCNCNACANGNTNTCAGGGNTNGGNGTGTNCA
NGANNCCCCTCTATATATGNNTTTGCGNGCGNCNTGGANNACCCACACCCCTATAGCNGGNGTGATAN
GCGCTGCTNNTANCAGANNAGGGNCCNCCAGTGTAANNTCTTTGAAGGNGANCCCNCCTCCNNTCTA
ACNTTCNCCTNNTGTAGAGNGCGCGGTGGAGATTCCNAAAGAGGCANAAANNCNTGNNGCTNGGTTN
GGANAATCNTCTGNTTNCNNTCGNTNCTTATTNANGTGNCCNTNNNGCCCTCNTTNAATCANANNATN
NTNTNNTTAACNNNCTTTAACCANCTNNGTTATCTTNGNGTTCCTGGTTNANTTNTCTATAAAANTCNT
NNTNCNTAGNGAAANAAAAATGNGNNAGAAACNACCGGTNNAGNNCNGCCTTNTTTTCTNTTNCTTN
NGCNNAAGTANCTNNCCTTCTTTNNGGGACGGAANNANCCTNTTCTTTCCTCCTNNTTTTTNCTANNGN
TAAGTCTATATTAAAGCCCNCNNNCCTTCCTANACCNNNTCCATGTCGCCAANATNACACAACCG
TTGANAANNTCTGGAGCTCCACCGCGGTGGCGGCCGCTCTAGAACTAGTGGATCCCCCGGGCTGCAGG
AATTNACTGCNAGGGNGNNACANGGNNNAANNCATCGGACCCTCNNTANGGGAGTGGTGGANCTNAA
CTCTCTGNGANGTGNCAGATTGGNACTCTTCTNNCTANACATGTATANCNTGAGANAGCGGNTGCGGA
ANAACACGGCACGTGCANGCGGACTCCNTGTTNTTGCTGCAGGGTGGNNAGGAGACGCAGCGCTACT
GCACCCGACACCGGNTTGCTTTTCCGCCAGGAGTCCTTCTTTTACNTGGGCATTTGGTGGTCACCGAGC
CGGCTTGTTACGGNGTNGATCAACGTGGACACCGGNACGNCNATTTTGTTCGGGCCNAGGCNTTTCCTC
CANNCCNTGCAACTNGGATGGNAAAAATCCATNCCANGGACNCTTCAAGGAAANNTCCGCCGNGAAT
NNATGTCCAATCCNCAACCAANAATTGGCCGNGGTTNTGGCNTTTTCGNAANCCCCTNNTGGGNNTCC
TAACTTTTTGCGGGGCNGGCAAAACCCCNAACANGGGGCAGGNATTTTGNAGGGNNGGCNTCCTTTCN
AGGGGCCTTTNACTAAGGTTCAATTGGTNAACAANNCCCCTTTTNTTTTATCAAAAAAATNTGGGGAA
GTGGCCGGTTTNTTTAANANANCGNCNATTGGNACCCTTGAAGGNTTTTTNGCCCNTTTANNCNCAAAA
NAAAAATTTTCCATTNGGGGGCCCCNCCGGNANGNTAAATTGAAGGTTTTTTAAAANTNNGGGAANTN
AAAAAAAATTTTGAANATGGGAAAGGCCCTTTTTTAAAAACCATTANTTGNTTANNTTCCCGGGGGGG
NGGNTNGCCCCCTAAAACTTCCNTNCCNCTCNGCTTTTTGGGGGNAGNNGGGGGNAAAAAACTTGGCC
CGGNNNTNGAANTNANNGAAACACCCTTNGGGCCCCCCCANNGNAAAAAGAACCTTTTCCAAAAAGG
GGNNAAACTTTNGCCCNNTTTCCAAANNNGGGNNGGGGANT
TTGANACAGCTGGAGCTCCACCGCGGTGGCGGCCGCTCTAGAACTAGTGGATCCCCCGGGCTGCAGGC
AGGATCAAGCGATTCCTNCCAAGAAACAAAAGCAGAATCGTCCCATNNCTCAATGGATTCGAATGAA
AACTGGCAATAAATCAGGTACAACTCCAAGAGAAGACATTGGAGAAGAACCAAGCTGGGTCTATAA
GAAGCAAGCTGGGTCTATGAGAAGTGGTCTTAACATGTAGACCACTTTTTTAAGCAGCCAGATCACAA
TGAAAACATCACTACTGTAATGCTTGGCCCATGATGTTATTTCCTCACTATCAGTCTGAGACCCAGCAA
TAAATATAAAACGTTGCAAAAAAAAAAAAAAAANNNNGGGGGGGGGCCNGGCCCCANTNCCCCNTT
NNGGGGGGGCNGTTTAAAANNCNNNNNGATCNNCNNACCNNNNCNNTANNNGCCTCNCNNNCCNATTC
NTTGAGNNNNCANNNNCCCTNNCNNCTTTNTTTCTTNANNCTNNNTGNNGGTNTTCTNTTNTTTTNNAN
CNTTTANTNCNNNNNTTNCAGCNNNNTNTTTNGNTTTNTTNCNNNCCTANNTNNNNTCNTTACNNTTTN
NCTNTTTTNNTTTNGGNCCNTATTTNTTTTNCTNGNNNGNTNTTTNTTNNGATTNNNNNNTNCTTNGGN
NNTNCNNNNNNNNNTNNNNCCNTCTNTATTTGANCGCTCCCCNTCATTCTTNNNNNNNNCTNNNTNNT
NTTTNTNNNNNTNCNNNNTTNCTNNNNANNCCCTNNTNCCTTTNNNCCNNTTNNATTNCCGTNNCTTT
NNGNNNCNCCTNNNATNNCTTNNNTNCNNTTNNNNNNNCTCNNTNNNGCTTNNNNNNNTCCNANTN
NNCTNNTCCATTCCNNNNCCCNANNNNNNNNNNTTTTTTNNNTNTTTNTNNNATTNTNTNNNCCCCTT
ANCNTCATCTNNNTNNCCTNTNNTTTTNNTNGGACCCG
NNNAAAAGCTGGAGCTCCACCGCGGTGGCGGCCGCTCTAGAACTAGTGGATCCCCCGGGCTGCAGGT
AAAAGTTGTTCTTCGTTTGGCTGGTAAAGTAACATGTACAACACTGATTCCTGCTCTGTGTATCAAGGA
GACATTGCCTCTGGCTGAATGAAGCAGGGTGTGAAAAGTGCTCAGAAACCAGAAGTTCCCTGTGCT
TTCACAGCTTTTGAGGCTTTTCCTTTTCACACTGGAGACTTTCTTCTGTTTTTAAGTGGGTACTGTGTG
GCTCAGAAATGGCTGCTGTGATGAAAATGCTCATTTGTTAGGAAAAAGAGGTAGCTACATTCTCATTT
TGAAAGGACCATGAGCTATTAAAAAAAAAACCTAAGATGTGGTTTTATTTACATGNCAGGGGAGCTC
TTGATCATGTTCNCATTGTTTTAAGATAAAGCGATTTTTTTTTTCCTGCTTTTAGGGTTAAAACCAAAGA
AATTTTATATTTTTATCATTAAAGGTCACTATAGTGTANNAAGGTNTGCTGAACCGTAGNGTTAGTGAN
NCCCAAAGTAATCCNTAGTAAAAGAGCAATATTTTTCTGAAGCTGGGATCTGGGTAATACAGAGTTGC
TACTCAAAAATCTAATAAAGCTCTTACTTCGTATTGGAAATTATAGAACCCTTCCTTTTTAAATAAAAG
CCCTTTTTTTTTTTAAAAAAANCNGGAGNGGNCCTTAAAATTGGAAAAANGCCTAAAAAAAAAATTTC
CCCTGGGANTTTTGGGAAAAAAACCATCCTNTAAAAACCTTGGAACCCCTGGNAAATTCCTTTTTGGC
CGGGGGCCAAAATTCCNAAAAACNTTTNTAAATTCCCNAAAATTTTTTGGNGGGGGGGAACCCTTTGN
TTTTTNANAAAAATTTTGNCCNAGCCNGGGNAAACAATCCANNGGGNTTTTTTNAAAAAAAAANGGG
AAACGCTNNTTTTTGTTTN
TTGAAACCGCTGGACTCCCCGCGGTGGCGGCCGCTCTAGAACTAGTGGATCCCCCGGGCTGCAGGAAT
TNNGCACGAGGCANTGNCCGTCNCGATGTTGNTGCCTAANANNANCCGGATTGCCATTTATGANCTCC
TTTTTAANGAGGGGGTGATGGNGGCCAAGAAGGATGTTCACATGCCCAACCACCCGGAGCTATCAGA
ACAAGANTGTGCCCAATCTTNACGTCATGAAAGCCATGCACNCTCTCANNTCACAGAGGCTACCNTGA
ATGGAACACGTNTGTCTGGAGACATTTGTACTGGGACCTNACCAACGTAGGGNATCCATTATCTCCGT
NGATTACCTCCACCTGCCCCCTGAGATCGTGCCCGCCACCCTGCGCCGGAGCCGTCCTGAGACTGGCC
GGCCAAGGCCCAAAGGTCTGGAGGGAGAGCGACCTGCAAGACTCACTCGAGGGGAAGCCGACAGAG
ACACCTACAGACGAAGCGCCGTGCCGCCTGGTGCCGACAAGAAGGCCGAGGCCGGGGCTGGGTCAGC
AACTGAATTCCAGTTTAGAGGCGGATTTGGTCGTGGACGTGGTCAACCCACCTCAATGAAGTTTGAAA
NGGGATTGGTTTGTGTTGAATAAACCTGTAAACNNGAAGAAAAAAAAAAAAAAAAAAAAANGGGGG
GCCCGGGACCCCAATTTNCCCCTTTTTTGGGGGGNNNNNNTTNANAANNNNNNNNNNNTNNNNNNTN
NTTNNNNNTNNNNNNNNNNTNNNNNNNNNNNNNNNNNTNNNNNNTNNNNNNNTTNNNNTNTNNTNN
NNNNANNNNNNTNNNNNTNTTTNNNNNTNNNNNTNNNNNNNNNNNNNNNNTTNTNTNNNNNNNNNNN
TNNNNNNNNNTNNNNNCCNNNNNTTNNNNTNTNNNNNTNNNNNNNNNTNTNNNNNNNNTTNNTNNN
TNTNCTNNTNTNNNNTNNNNC
GGGTNNGNGGCNNNTNAAATCNNNTNNNNNNNNNTCNNNNNCNCCCNNCCCCCTNCTANCNTTCTNT
TTCTTTCTCTNCCCCCCCCCNAANGGNNGANGNATGAGTAGATTGACCNNCTTTTGAAGNCNNCNNTG
GAGNTGCATCACNGNGNCTACCTTTNNCNTGNTCTTNTTGTCGTTNTCCTCCCANCACAATTCCAGNAN
ATNATTANATNTTCACATTACATNANGCNAATCCNTNATTANACTTCNNGGGCCTCNNTCTCTCACGN
CTTNATNCTNCCTCCTNATACNNTTNACCGCCANCACTNNCNTNNCANCNCATACTANNCCAANNTNN
CACTNCCCATATNNCTGANTTTNATNNANTTTACATNGTNTTNNAAATNTTTNCTCACACNTTCNTAC
NANANTNTTTNNNTCTNTCNCNTTANCATANNTCATCTANTCNCTNACNNTCATNTTACGCTTNGTTNN
NCNACTCTATCTTCNNCNTNATTNACNNTATTCTNATANCNNCNNNNTNACGTNANATAATCTNNNT
TTACTNNTNNGNCTNTTGTNNGCGACAANCTNGNTTCTCTACNNTNTANCTCACTNNNAACNCTANC
NNTTTANNNCTNNTGTNNTCCTANNTNNTAATCTTANNNTNCTCACTTACTANCCTNGNGNTANACAT
ACNNCTTAAAACCCTNGANNNANCTNTNCTANAACCTCACNTCNGCTCTNNNAANCNNNTNCNGNN
NAAGNNAGAANNNAANTNGCNTNTTTTANTGACTGNTNANNNCGANNCTAAGNTAANCTNNTATTNN
NANTNNGANACAATNTNNGCGNNATGNAATANACTNNCTTNCTNNAGAGGGNNTCTAGCAANNGCGG
CTGNTTAACACGNNTGTNNANANTNCCANNGTNTGANAATNAAGACNACTANNNTCATNNACNNNTT
NGNNCACNACACTCTNTNNNNCAGNTNCNNNAGNCNCCTNNCNTCNACNTTNCTCNANNTGTNNNA
NTANNNNNTNTTNNGATACNNCNCNANAANCTNTNNNNATCNGCTNNTCNNATNNAANANTNATAGT
TANTANCNNNNNATNNCNNTGGNNTNCGCANANAGANCTCATNNTCTCNCNTNGNNTANAGTNNTGA
NNNANCNTATATCNCNCNATCNCGACGTGCAAGATANNNCAANNNANCAANGNNCNNNTNTNCTGATCT
CACNNANANNGACGCTNAATANNCGNTATCTNACTCTCATTCGNGNTNNANCNCATANNTGCNCNTN
NGACNCACNANCNANNGATNANANTNNCANNNNTNTNNNNACNCNANANANTTGANGACNCNNGNTAT
ACTNANACNANACAGACNNGAGCGTCTANATTGTNATANACGACACNNNGATCGANATANNNCNCGN
GNANANNTNTCNNNCGANTCACATTNTGTTNNACACGANACATNTAANNACACNGGTCGTGACTGNC
NNGTGTCGNCACGANTATCGNNANANACNANTCTACATATATGTANACNCGACNCACGTAGACNNAA
GACANGNGNNACNTGANTNCNTCATATNTCGANNCNANNCTAAACNAAANGANACNATANGTNACG
NGACNACATGNACTATTNTACANGATATNAGNTGCCGTTAAANACTCNATGTNTTNNGNCGNTNTNAT
NATGNGNC
NCAANATCTGGAGCTCCACCGCGGTGGCGGCCGCTCTAGAACTAGTGGATCCCCCGGGCTGGAGGAA
ATTCGGCACGAGGATGGCACGTCCCCAGTGTGGGGATGTGCCGTGCTTCCTGAATTAGTGTATTGTAG
GAGTTGCTGCCCCATCTGCTCTTCATTGTGAGAGAGGTTACACCCACTTATAATCAGTATTTTCCTGG
CACTAAAACAGCAGGGCTGGTATTTGCTGCTGCTTTCCCAGGGCAGGGTCCCTTCTTTTCGGCACTTTT
GAACACTTGCAAGACACAGCCCTGTTATCTAACGGGTGGCAGGCATGTGTGTGTGCACTCGCTCTTTGC
CACTATTTTGCCCTGCTCCATGCCAGAGAGGCCTGTCCCTGCCAGGCCCTGCCTTCCCAGCCCCAACTT
GGGACCAAAGTGCAAGACGGGATCACAGGTTGGGGTGTCCAAGTGACGCCTCTCTATAGTGCTTCCGT
GGGCCAAGTCGACACCAGCCCCCTANCGGGGTGGGATGGGCGGTGCTTAAAGAGGAAGGGGACCAAG
TGTANCAACTTGCCAGGGACCCCACCCCTTCCTCACATTCGGGCCTGTGCAAGTGGGCGTGGGGATTC
TNCAAAGGGGCCAAANGCCTGGGCTTGGTCGGNATTCTCTGCTCCTTGCTCTTTATCTGGGNCACATGC
ACACTTCTAAATGCANAATTGCTTTGAACTTTAAAACTGGTCAANTTTGGGTTTCGNTTTGGGACCGAA
TTTAAAAAAANGTTTTTANTNGGGGGAAAATTTGGGGGGAAAAAACCNGGGGNNNGGGGNNNCCNGGTTT
TTTTTTTTTTTTTGGGGAAAANANANGGNCNNTTTNNCGGAGGAAACGCNTTTTTGNTNCNANTTTTTN
NGGGAANNGNCNAAAANCNNTNCNCCCCCNNNNAAANCCNTTGGNCCNGGNAATNAAAAANNGGGG
TTNNNAANNNCNNTNGGGGGGCGGGNNNGNTTTTTTTGGGNACCTTNGGGGG
GCCCTATANNGAGNCGTATNACAGGGGCGGCCGCTCTAGAACTAGTGGATCCCCCGGGCTGCAGGAA
TTCGGCACGAGGNCNTGANNTATGANNCCNANCGNGTGANNCATAANNNANAGTGAGTCGTGCGNNT
GAANCCNGCNCNTTNAGTTTTNGNAATGCTGNCTTNAAANANNGCNNGTTCANATCTAGNGNNACAG
NCNGNNNAACTNTTNNACAAANCTTATNATNTNATTCTGANCGCCNCTATTANTNNTGNTGNANGGGA
NGNNNGAGNTAAATCCCTGTTGANGAAAATTAANTAGATATTCTNTGTGGTNCNANATTAACAGGAA
GGCGCGCCCGATGAATAGGCCGTTTTACTCTGCCACAAGCGTCAANCGTTAATTATACACNNAANCAG
AANGCNATTTTGNTTTCCACTNAGANAAGANATATACTTTTCNAGAGACNTAACNGAAAATTATAACC
ATTNGNCAGAGGNCCANNCTTGAGCACCTTGNAGACNANNANGCTGGGCTCANTNTCCCCACNAGAC
CNCCTATAGTGNTTTNGTNTTNGANNGGACACCAACNTGNTNTCCNACTGANCCTNCCTNACNNTACCT
NNCATGACGGAAGGGGNGGAGGTGGNNAAGNCANTANTGGANCANTGCGCNNGNAATAATTATAAT
ANTATTTTCTTTTTTNCANCCCGTACACTTNGGGAACTTNCCTANGAGANAGGTGCNCNCCNNCGNCTT
ATTTCCAAANGGCTCNCGGGAAAGGGNGGCCNCATTTNCCCNTCTTCCCCANNNTTGGANGGAAANCC
CANANTCAANNCGCCGNCNNTNNNAGNACCCGGNAATTNTTNNCCGCNTTATNCTCCNTAGGNNCNC
CTTNCNCCTTTCCNGGNNGGCGNTATNTTTATCANNCNCNNAANCNNTNNTNTGGTCNCTTGNTCCCAT
TGTTCCNTNGGGNNGGTGGGAANTNNGGNCNCTCTNNGCTTTGGNCTCANTTCACTCCG
GGNGCGCGGNGNGNCACTTTCNNTTTTTTTNATNNNNTNTTTTTNCCTCANNNATNCTTTCTNTCCTTNN
ANTTANNNGTAGNATCACCANNCCCANCCTAGGGGGNTCTTGGAGTNANTGTTNNCNNTTGGGNCCN
ATANGNNTCGNATTACANTGGTNNCCGCTCTNCANCGANNGGCAANANCNNACNCTATAGNGATTTT
CNCGGNCGATGCNTGTTTTCCTNCGNNCGNCTNCGTNTCNNNCCTGCATGGGGGNGACNNNTNTCNNN
TTCTGTATCNTTTNCTNNNCNNCCNTANNCNGNCCNNTCCTTGACNTAATATATNNNTCNCNAGATAN
TNNGNTCNNNTCCAGTNCNTNCTCNNNCNTANTATTCCNTATANACCCCNNTNGTNCNNTTNNATTN
CNNTTCCAANNCNTTNTCTNNNTGNNCCNTTCTNNNTCTTTNTNCNGCCTATTNTNNCNNGCNCGATTN
NCTNCGTNATTTATTTNCNCNNTNGNCTCNNTATCNANCCCNATTTNNNNTAGANCCNNTNNTAGCCNC
NNCANCTTNCNCNNNTGCGCNNNNTNTAGGTGNNNNNCGACTNNNTTTANNNNTCTATATTCAAAATN
CTTNTNTTTCTCNAATTNNTNNTTCANNTTTTCNCTNNNNCCCTATTTNNCTNTTTTNNANCTNTTNTN
TNCTNTGNATNTCNCATGATNTCTNGGNTTNTNNNCGNNTTANCTNATNCNNATNNCTNNATNTANNC
ANNTCGNTAGTNNTTCCNCNCANNTNTCGANGANCNNNTNTAANNTTNACTATNNTACTTCTATCGCN
NCNNCGGCNTCTNNGTNANNNCTCCNTTTTTNNNNNCTCNNTATTGNTNGTCGCNNAANTTNNNAGTC
NAAGNGANTNNNNCNNAANCCNNTCTGCCTCTCNNNNCNNCACNNNNTNGATAATTNNNCCACATNT
CNNCNNGCANACCCNTTGTNATGGANNTTAGNTGTATTAGNNGATGACTCGTNCTANTCTTTCTNTTN
CGNNCCCNNTGGNCTTNNCNTNNNNNTGNATGAAGATNNCCCCTTTNCCCCTTNTNNNTCTNGCCTCT
GACTGTCCNNNTANGACCCTCTCCNAGGNACCTNTCNACTNTNTACTNCTCCTTCCGTTANNNCTNCNC
CNCNTGNTNANCACCTNAGCANTNNAACTNCNGANTTATTNCTTCTNTGNNNTCTCTNCTNCCNNTNG
NCANTNNCTCAAGACCNTCATANTTNGTANTCNCCNCNATNTCNTNTCTAGNAATATATGTNCTNCTTG
ANAGCANNCCTGATTANCTCCGTACCNNATGNGNGGACTNTCTATNTATGTTCTNGTNTACTATAGNN
TNTNTCATGTACTTTTANTTCAACANAANTACNCNNCNNNCNCTTATCTAGNCTNNTCCNTNAGTNNGT
NTGTTCTTAAGATNACCCACCAAAGNANTCTNNTNTCGNGATTNTGNNNATTNNNTANCTCTGTACTTT
AAGGTATTNCTCTCTCNNANATACGTACNTNTCCGNTANCNNTCCC
GGNGCGCGGNGNGNCACTTTCNNTTTTTTTNATNNNNTNTTTTTNCCTCANNNATNCTTCTNTCCTTNN
ANTTANNNGTAGNATCACCANNCCCANCCTAGGGGGNTCTTGGAGTNANTCTTNNCNNTTGGGNCCN
ATANGNNTCGNATTACANTGGTNNCCGCTCTNCANCGANNGGCAANANCNNACNCTATAGNGATTTT
CNCGCNCGATGCNTGTTTTCCTNCGNNCGNCTNCGTNTCNNNCCTCCATGGGGGNGACNNNTNTCNNN
TTCTGTATCNTTTNCTNNNCNNCCNTANNCNGNCCNNTCCTTGACNTAATATATNNNTCNCNAGATAN
TNNGNTCNNNTCCAGTNCNTNCTCNNNCNTANTATTTCCNTATANACCCCNNTNGTNCNNTTNNATTN
CNNTTCCAANNCNTTNNTCTNNNTGNNCCNTTCTNNNTNTCTTNTNCNGCCTATTNTNNCNNGCNCGATTN
NCTNCCTNATTATTTNCNCNNTNGNCTCNNTATCNANCCCNATTTNNNNTAGANCCNNTNNTAGCCNC
NNCANCTTNCNCNNNTGCGCNNNNTNTAGGTGNNNNNCGACTNNNTTTANNNNTCTATATTCAAAATN
CTTNNTNTTTGTGNAATTNNTNNTTCANNTTTTCNCTNNNNCCCTATTTNNCTNTTTTNNANCTNTTNTN
TNCTNTGNATNTCNCATGATNTCTNGGNTTNTNNNCGNNTTANCTNATNCNNATNNCTNNATNTANNC
ANNTCGNTAGTNNTTCCNCNCANNTNTCGANGANCNNNTNTAANNTTNACTATNNTACTTCTATGGCN
NCNNCGGCNTCTNNGTNANNNCTCCNTTTTTNNNNNCTCNNTATTGNTNGTCGCNNAANTTNNNAGTC
NAAGNGANTNNNNCNNAANCCNNTCTGCCTCTCNNNNCNNCACNNNNTNGATAATTNNNCCACATNT
CNNCNNGCANACCCNTTGTNATGGANNTTAGNTGTATTAGNNGATGACTCGTNCTANTCTTTCTNTTN
CGNNCCCNNTGGNCTTNNCNTNNNNNTGNATGAAGATNNCCCCTTTNCCCGTTNTNNNTCTNCCCTCT
GACTGTCCNNNTANGACCCTCTCCNAGGNAGCTNTCNACTNTNTACTNCTCCTCCGTTANNNCTNCNC
CNCNTGNTNANCACCTNAGCANTNNAACTNCNGANTTATTNCTTCTNTGNNNTCTCTNCTNGCNNTNG
NCANTNNCTCAAGAGCNTCATANTTNGTANTCNCCNCNATNTCNTNTCTAGNAATATATGTNCTNCTTC
ANAGCANNCCTGATTANCTCCGTACCNNATGNGNGGACTNTCTATNTATGTTCTNGTNTAGTATAGNN
TNTNTCATGTACTTTTANTTCAACANAANTACNCNNCNNNCNCTTATCTAGNCTNNTCCNTNAGTNNGT
NTGTTCTTAAGATNACCCACCAAAGNANTCTNNTNTCGNGATTNTGNNNATTNNNTANCTCTCTACTTT
AAGGTATTNCTCTCTCNNANATACGTACNTNTCCGNTANCNNTCCC
TTTGANACNGNTGGAGCTCCNCCGCGGGGGCGGCCGCTCTAGAACTAGTGGATCCCCCGGGCTGCAGG
NANNAACTGGAAAGGNNTNATNANGNNTAATNNNTTNGACCNCCCAATGGGGACAATCTGCTGCAAT
TTATGACACNNTAATATCAACNNAATATATATATACNATATATATATATANATNCTNTGTAANNACTA
TNTTGAAACANTTATTNGCANNTGTTNTCTTTTATTTGGGGGGCNNNNCTNCNTTTTTGAANNACACGN
NCTACCCTTNTNTTNCTCCTNCATNNCTTTCCNATCTCACNGANATGNATTTTTCNGNNTCNTCAANNT
TNCCNGGCTCNNCTCTNANNCNANNNTTTTTCCACATTTTCTCTACNCTCNCTANTACCTATNTCANGT
TGNNCNCTTCNTNTCACNTTCTTTNNNACTNNCCTATTTAATAGTTTNTTTNAATTATCTANCTNNCCAA
GNAACCNTCNGTTTCCCCTNNNTCTTNAATTCTCCCTTCTACANTTNTANNCTTNNACCTCCTTTTNTNA
NNNNNTNNNACCTTNNNNTTTGGTNTTCCTNNTTNTTTCNNATNATNNTTTNTCNNTTNCCTNATNTTA
TAATCAACAGNGTNNNATAAATNNNACTNNGNCCANNTCCCANCCTTGTCATCTTAANNCCCCNCNNT
TTTTTTNTTNAACCATCNNCNCNNNTNTCTTGNTTCTATTTTTTANTTGCCNTNCCNTANCCCGNGNTAC
TNNTTATNAANNANNNCTTANCNTTTANCTTNTNNTCCCTNANNATTCCCNNTGCCTNNNNTTNCTAN
NNTANTNNANNNCTNNAGTTTTCTNCCTNTNCANNTCTNNNAACNAANTCTNNCTACNNNNNNNTNTT
TNCTTTACCTNTCNAANCCTTNTCNNACCTTTCCNNCTTCTACCGTCNTCNNCCTTCACNTTNNTGTTCT
CTTTCTAANCNATCTCGTTCNNCTCANTCNTNNCCNTNTNNCNTCTTNNTCANACTCTTTTNTTCTCCNT
CACNANCNNCTCNNCATNTTNNTNTNNCC
TTAANCACAGCTGGAGCTCCACCGCGGTGGCGGCCGCTCTAGAACTAGTGGATGCCCGGGCTGCAGGA
GCTTATTGAATGAAAACTCCAGAGACTGAAAAACCAAACATGAGGGTAGCAGGTGCTGCAAAGTTGG
TGGTCGCTGTGGCAGTATTTTTACTGACATTCTATGTTATTTCTCAAGTATTCGAAATAAAAATGGATG
CAAGTTTAGGAAATCTATTTGCAAGGTCAGCATTGGATGCGGTTGTACGTTCTACAAAACCTCCCAGA
TATAAATGTGGAATCTCAAAAGCTTGCCCTGAGAAGCATTTTGCTTTTAAAATGGCAAGTGGAGCAGC
CAATGTGGTGGGACCCAAAATCTGCCTAGAGGACAATGTTTTAATGAGTGGTGTTAAGAATAATGTTG
GAAGAGGAATCAATGTTGCCTTGGTAAATGGCAAAACGGGAGAACTAATAGACAGCAGATTTTTTGAC
ATGTGGGGAGGAAAATGTGGNCCCANTTATTGAGTTCTGAAGGCCATACAAGATGGACCAATAGTCTA
ATGGGAACATATGATGATGGGACCANCCAACTCAATGATGANGCCCGCGGCTGATTGCTGAATGGGG
AGTACATCTATTACTCATCTTGGTTCAAAACACTGGGGTCTTNTGGGGNGGAAAAGGCATTAAAAACA
AAAGCCCTTTTGACNGCCCCNTAAGGACCATTAGGGCCCCAANTAAATTTGAANGGATGGCCCTGAAT
TTTNGGAAATGGAAAGGATGCCTTTCCCCAAAAGCAANACTGGCCGTGGANAAAAANCCGAAGGGGG
NCCCCCNTTTTCCTTGTTAANGGGGNAAAGCCCCTTNCCCAAAAAAACTTCCNTGGTAAATNTTTTTAA
ANAACNTTGCCCCCCCNTTTNNGGGGGNGNGCCCTTGTCCCCNTNTTNTNTTTTTGGAAATTCAGN
TNNANAAAAGCTGGAGCTCCACCGCGGTGGCGGCCGCTCTAGAACTAGTGGATCCCCCGGGCTGCAG
GAATTCGGCACGAGGGTGGCCTTCTCCTCTCGCCGCCATGGCGTGTGCTCGTCCACTGATATCAGTGTA
CTCCGAAAAGGGGGAGTCCTCTGGCAAAAATGTCACTTTGCCTGCTGTGTTCAAGGCTCCCATTCGAC
CCGATATTGTTAACTTTGTTCACACCAACTTGCGCAAAAACAACAGACAGCCCTATGCTGTCAGTGAA
AGCAGGTCATCAAACCAGTGCTGAGTCTTGGGGTACCGGCAGAGCTGTGGCTCGAATTCCCAGGGT
CGAGGTGGCGGGACTCACCGTTCCGGTCAGGGCGCTTTTGGAAACATGTGTCGTGGGGGCCGCATGT
TTGCGCCAACTAAGACCTGGCGACGTTGGCACCGCAGAGTGAATACGACGCAGAAGCGATACGCCAT
CTGCTCTGCACTGGCTGCCTCAGCCTTACCAGCGCTGGTCATGTCTAAAGGTCATCGTATAGAGGAAGT
TCCTGAACTTNCTTTGGTGGTGGAAGATAAAAGTTGAAGGCTACAAGAAGACCAAGGAGGGTGTGTTT
GCTTTCTGAAGAAACTTAANGGCCTGGAATGATATCAAAAAAGGTCTATGCCTTTTCAGCGAATGANA
NCTGGCAAAAGGCAAGANGANAAACCCGTCNCCGTATNCAACGCAAGGGGACCCTGCNTCATCTAT
NATGANGGACAATGGGTATCATTCAAGGNCTTNAAAACATTCCCTGGAATTACCTTTGCTTAAANG
GAAGCCAACCTGAACANTTTTGNAAACTTGGTTCCTGGGGGGGCACCGGNGGGAACGTTTTTTGNANT
TTGGAACTGAAAGNGCNTTTTCCNAAAGGTTAAAAGAACCTTTTTTGGGCCCCTTGGNGGTAAAACAC
CCCTCNCCCTTNAANAAA
TTTGGAAACCGTTGGACTCCCCGNGGTGGCGGCCGCTCTAGAACTAGTGGATCCCCCGGGCTGCAGGTA
AAAGTTGNTCTTCCTTTGGCTGGTAAAGTAACATGTACAACACTGATTTNGGNGCTCTGTGTATCAAGG
AGACATTGCCTCTGGCTGAATGAAGCAGGGTTTGTGAAAAGTGCTCANAAACCAGAAGTTCCCTGTGC
TTTCACAGCTTTTGAGGCTTTTCCTTTTCACACTGGAGACTTTCTTCTGTTTTTAATGTGGCTACTGTGT
GGCTCANAAATGGCTGCTGTGATGAAAATGCTCATTTGTTAGGAAAAAGAGGTAGCTACATTCTCATT
TTGAAAGGACCATGAGCTATTTAAAAAAAAAACCTAAGATGTGGTTTTATTTACATGTNAGGGGAGCT
CTTGATCATGTTCACATTTGTTTTAAGATAAAGCGATTTTTTTTTTTCCTGCTTTTAGGGTTAAAATCAA
ACACACTTTACNCTTTTATNATTNGAGGCNNCTTATAGTGTANGTAAGGGTNTGCTGGAACCGTANTG
TTANNGGAACCCCNANANGTAATNCTTATTTAAANGGAGCCNTATTTTTTTTNAAACCTGTNGATCTTG
GNTAANTACNAGNANGTCNGNNTTANTCNANAAAATTTTNAATAAAGCNCTTTAANTTTCNNATTGGG
GAAAAATTTATAAAAANNNNTTNTTTTTTAAANNAAAAGCCCNNNNTTTTTTTTTTANNAAAAAANCA
GGGANTTNGACAATTAAAATTTTGTATNATATNGGGCCNTAANANAANAAAATTTACGNGNGGGNAT
TTTTTGGGGAANNATAAACCANNCTATTAAAANACCTTTGGAAAAACTNGGAANAAATTTCANTTATG
GGGNTGGGNGGGNNNTNTATTCTNCAAAAAATCTTTTNTTAANTTTACNATANATNATCTTGNANNGN
GANNAAGCCCTNTTGTCTTTTTNACTAAATANTCTTTGNCCAAGNTTNNGNGAANAAAATATTNTAAN
NNTNT
GNCCTTTANTTANGTGNANGTTCGGTAATTAACNNGGGGGANTNCGTNNTNTTAAANCTTCGTCGCCT
TCNTTCNNNATGCNCTNGNGANGCANTCCCTTGCACCTCCCNAGANTTTCTTTATCCTACCTCANNNCN
TGACNNGCANTTTCCNANNATTATNTNGGGTATNTCCTNTATGCACAANNCNNNACNTTACTNGNGTAC
TTATTCTNGCANNTGATCGTACCTCTTNCGCTTCANTTNANACCATNTNNNNAANTTCGNTTGCTCCCC
CATTTGGNTANCTACNCCNGNNANAGATTNTCTANATANNNTNNGTTGGCCCATANCTGNTGTTTGNNC
TNTATCNNNCTCAGGTCTNTGAGNATGANAATTNATNTTCGNTNANNANCGNTNGAAGTCCTATCACT
TTANGTTNGNNAAGNGNNGNNCTNTNANTCAANGNNNTANNNAGGCNTGTNNNGNGCTGTCNTTCNN
ANNTACGNTGNNNAGNNNANGCNCTCANGCCCNTAATAAGNNNANGGGGGTTNTNTGNNTAAANTCA
GNTNGACNANNTNTATNTNCCNCNCNGNCTNGGGGGCCNGTANTANNTNNACTNAGGTTAAAATNGG
NNNNGGNNNNNNATANGTGCCTACANTGANNNNTGCNAAGGNNNACNNTTAANTATNNNGAANNAA
ANNNANGNGNGTNGNTNTNTAAACTANTNACATNNATTNNNNNTGNNAGTANTTCNTNNNTNGNACG
AGTCGGGNNTGGNAGTNTANCNNNNNNTCTNGNANGNNGNNANGNGNTNNNNNATNTTATTNTNTNT
NGNGNGGCTNNNANTAATAANTTANNNNGNCATNNGCNNNGGNNANNNGCCGNCCTTTTAGGATANN
NNNNGGGCCGNNNNCAGNTANNNGNTAATNNGNNTCNGNNNGCGGANNNTGCANGGCNGGANCTNA
NNNGGNTNATTANACCANNNTGNNNTGGTATCGTNNNNNNTGNCTANTNANNGCGNNAGGGGNAGTN
GCGCNNCGNATANATGAATNNGTNGGANNCAGGAGNGTANTACTANTGCNTTTANCANGNANNTAGC
NGAGTCNCNTANNTGGTGNGNTGNTGTATTATTANCAGTTNNTNTTGTGAACTCNNCAAGANANCNTA
TGCAGAGNAATNTANCGTTNTANNGGTGNTNNAATNCNCNNGNGCANACNCNCGNGTNCTCNNCNAG
CGCTNNNTACTGNNNACATGNANTCACCGCNCATANTGNATCATCTNNNTGATANCTAGANACACTTG
TNTGNTANNTNACCG
GNGTATCTGGAGCTNGACCNCGGGGGCGGCCGCTCTAGAACTAGTGGATCCCCCGGGCTGCAGGAAAT
TCGGCACGAGGCTCTGTCCAAAGGGTTCAGGAAGAGAACATGATGGTGAGCCACAGCCCTCTCAGGA
TTCGCTGGCATCTCATCTGAGGGTGGGAGGGCATACTGGGTCTTCCGTCTCCTCCAAGCTTTCTTTGTC
TGTCTCAATCTCTTGGCCTGGCCGCTGTCTAAGAGGGGATGGCAGTTATCTGGGCTTGTCTCTGGGATG
GCCGCCATCTTTAGTAATTGAGAAGGTATTAGATAAATCAGCTACGCCGCCTAGAGATNTTTCCTCTAA
GTTTTCAAAAGAGAGCTGGGCTGTAGTTATCCCAAACGTGCATTTCTTCAAAGTCGAGTCATCCTCTGG
CCTTTTCCTTTGTGACTCCATACAAAGAGTCATGTTCTGCCCCAAAGGCGTCCATTCCAAGCCTGGACT
TGGGCTCCCCCGTGAGAAATCTTCCCAACAGCTTCTTGGCCCAAGCTTCCCAGACCTCCTCCCGTGGAA
CCTCCAGCCCGAGTCCCCGTGTACACGTGGGCTGTCCCAGGCAGCCAGCATCCCTTGTAAGTTCCCTCT
TTGGAAATGGTGTGTGGGTGTTCAAGTTCTGTGTCTGGTGACTATGAACAGACAGTGACTCTTCCTGTG
ACCTGTGGGAAGCTGTGAAANCAACTTCTGGAACGCCGANGAGCTTGTTTGCTTTGAACCGGGGAANA
AAAACTGNGTTTTGAGGTTTANCTGAAAATTAAGGGGGAAAAAAAAAAAGTTNATTAANGTGACTNG
TAATNGAAANCTGGTNTTAAAAANAACTNTGAAACCCCCAAAAAAAAAAAAAAAAAAANTNGGGGG
GGGGGGCCCGGGNNCCCATTTNCCNTTAANNGGGGGNGNNNNNNTAANNNNNNNNNNNNNNNCNNN
NNNNNNNNNNNNNNNNNTNNNNNNNNNNNNC
GCCCTATNTGGAGNTGCATCNCGGGGGCGGCCGCTCTAGAACTAGTGGATCCCCCGGGCTGCAGGTTT
GGACTCTGAGACTGACAAAAGGGGTACCATGCTGTCTGGCACTGCNGCACNCNTTTGCTGAAGAAAA
GTACCCACCTTGANTGGTAATCCCCANNTTCACTGANCCTTGTTGCCCTGNACCTCACTCTAGGNATGA
AACCNNGATGCGNGGCTNGTNCATAAACCCTATTCTTANTATTCCNAAGGAGCCANTNNTTNTNTACT
GAAAGCATNNCCNATACTACCTGANCACTANTCTAAGAGNANANNCTNNATACCATATNNAATATTN
ATAGGCNATTTTANGAAGCNACGGAANGANNACAAGTANNTCATTNCTGATNCTNNNTGTANCAACN
NTGTTTACATNCTACTGCCCACANTNTGNNTCTCNTNCNTTGNAGGGACTNNTCCTNANNTNAGNNNA
NNGTGANTCNNGNTNTTTTTANNGTTTAANTGCAAAAGGCCTGAATNNNNNNNACTGACTTNGNTNAN
CTCTAGCCNAANCCCTNCNNNNTNCGNANNGANGNCCCACATAGNCAGNTNNCTTTTGACTCGTCCNN
ANCGCCTTCCTNCTTTCNCGACANNANNNATACCCTGNGGCCATNCCCTNGANNGGCGNTTATGANNG
CTTTNGANTCTCTNGTTCCTCCAACTCNTTCTNGGGAGNAGCCNTGGNNATTNNTNNNANTTNNGGTA
CTGCAAAGNGNTCNNCTNTAGGCCTTCTNNTANTTNTCTCCCTTNNGTNCCAGCNTNNCTNTNGNNAN
GNCTANCANNGNNCTNANTCCNGGTTTTAACNTCTCNNTTTTNCAAAAANGCACANNNNNNCTTTGGNN
ANNTTGCNCTNTTNCATNNAATGTCNNAATTTTNGTTTTTATATACANTCCATTTNCGGNAAAANTCTT
CNCATGGCNTCNNCATTNCTTCCATCTNCTTTNTTCGCTNNNTNTGNCTNNNATTGCTNCTATACNTCN
CCATTNNTGNNTNNANTGTTAANNNACC
NCAAAACTGGAGCTCCACCGCGGTGGCGGCCGCTCTAGAACTAGTGGATCCCCCGGGCTGCAGGAATTT
CGGCACGAGGCTCTGTCCAAAGGGTTCAGGAAGAGAACATGATGGTGAGCCACAGCCCTCTCAGGAT
TCGCTGGCATCTCATCTGAGGGTGGGAGGGCATACTGGGTCTTCCGTCTCCTCCAANCTTTCTTTGNCT
GNCTNAATCTCTNGGCCTGGCCCNTNTNTAAAAGGGNATGGCAGTANTNNGGCNTTNTTTTNGGAANG
GCCGCCNTNTTNANNANTGGAAAAGGNNTAAAATAANNAACNTCCCCNGCNTAAAAATTTTCTTTTAA
NTTTTAAAANAAAGAGNNGGGNGTGTANTTATCCCAAACGCCATTTNTTCAAAGTNCAGCANATCCTG
TGGCCTTTNTCTTTGANACTCNANACAANAGAGNNGTGTTGTGCCNNANGNGCGCANATTNNANCCCN
NGCNCTNGCTCNCCCNCGAGANANATTTNCNANACATTTTTTTGGCCCAAGNTTCNCAAANCTTCTCCT
GTGAAACCTNCANCCAGACCCNCGTGNACACGNGGGTNTGNCNCANGCACACCANATCCCNTGAAAG
NCNCTTTTGAGAAAAGGGNGNGGGGGTGACAANTGTGGGTGGTGGCGATAACAACANNCGCNCTCCC
CTGCACCTGNAGAGCTGAAAAACNCTGTACACCCCAAAAGCTTTTTNTTTCACCGANAAAAAANCTGG
NTTTNGANTNAGCTNAAATGGGGGGNAAAAAAANGTTCNTNNGNNGCTGTNANGNCCCCNNTTATAA
AANNCCNNGNCCCCCNAAAAAAAAAAAAANNCCNGGGGGGGGGGGCCCCCNNNNNNNNNNTTTTTNN
NNNNNNNNNNNNN
NCAAAACTGGAGCTCCACCGCGGTGGCGGCCGCTCTAGAACTAGTGGATCCCCCGGGCTGCAGGAATT
CGGCACGAGGCTCTGTCCAAAGGGTTCAGGAAGAGAACATGATGGTGAGCCACAGCCCTCTCAGGAT
TCGCTGGCATCTCATCTGAGGGTGGGAGGGCATACTGGGTCTTCCGTCTCCTCCAANCTTTCTTTGNCT
GNCTNAATCTCTNGGCCTGGCCCNTNTNTAAAAGGGNATGGCAGTANTNNGGCNTTNTTTTNGGAANG
GCCGCCNTNTTTNANNANTGGAAAAGGNNTAAAATAANNAACNTCCCCNGCNTAAAAATTTTCTTTTAA
NTTTTAAAANAAAGAGNNGGGNGTGTANTTATCCCAAACGCCATTTNTTCAAAGTNCAGCANATCCTG
TGGCCTTTNTCTTTGANACTCNANACAANAGAGNNGTGTTGTGCCNNANGNGCGCANATTNNANCCCN
NGCNCTNGCTCNCCCNCGAGANANATTTNCNANACATTTTTTTGGCCCAAGNTTCNCAAANCTTCTCCT
GTGAAACCTNCANCCAGACCCNCGTGNACACGNGGGTNTGNCNCANGCACACCANATCCCNTGAAAG
NCNCTTTTGAGAAAAGGGNGNGGGGGTGACAANTGTGGGTGGTGGCGATAACAACANNCGCNCTCCC
CTGCACCTGNAGAGCTGAAAAACNCTGTACACCCCAAAAGCTTTTTNTTTCACCGANAAAAAANCTGG
NTTTNGANTNAGCTNAAATGGGGGGNAAAAAAANGTTCNTNNGNNGCTGTNANGNCCCCNNTTTATAA
AANNCCNNGNCCCCCNAAAAAAAAAAAAANNCCNGGGGGGGGGGGCCCCCNNNNNNNNNTTTTTNN
NNNNNNNNNNNNN
TTGANACCNTCTGGAGCTGCCCGCGGGGGCGGCCGCTCTAGAACTAGTGGATCCCCCGGGCTGCAGGA
ATTATTCGGNACGAGGNNNNTTNNATGTNTGNACCTGGNCGNTGGGCCCNGGGGCNTANNCCCCNTACTN
GGAGTNNTACNATNTTCTTAATTTGGAACTTCAANAGTCCCTACAAGGGTNNAAAGCCCCTAACCNNA
TCTGGNCCAGGGTGGCCNTCNCNNCNANCTGGACCNTCACTTNTGACTTNCATGAAGGGNATCCCGGC
TTTTTCTTTGGGTCCCCTTGTNGGGTTANCCNTCCACTTACCACAAAAANAAAGACTTTGNGGGGAAG
GGCCTGNACCCCAATCNNCCTTNANTTTTTCCCTCTNAAAANNTNGTTGGAAACCACAATTTCCATTCT
NGCNAGCCCCTNGNGAAGGAAAANNAGCAAGGNCTTNAACNAAAAAANGGTNCCTTTTGNAAAGNN
ACNNNNAGAAGGTTTGTTTTTGANCNTTNNNNTAAACCATTNCCTTTGGTTATTTCNCCTGNTTCCNTT
ACTGGGNTNATTTTAAANNNCNTTTTTANTGTNGGCTGTAAACAACTTTCCCGCANTTTANTTCCNTTT
CCCATGAAACCNTTTTANTTNTTGCTNAAATNCTTTAANNCCATGNGGGANGGCNTNTAAANANTGGN
NNNCCCCTNGGTGTTTTTANGAANAAAANNAANGNANACCCCAAACCCCCCCTTNGCCANCCCATTTT
TTTTTTTNAANNAAANTTNTANCCAATGGGAAATGGNGGGNTAAAANNANTTNTNNNCTNNGCCCCGC
CCANTTTAAAAATNGGAAAANTGGGGGGNAACTTAAAACCACCCNNCCCCCTNGGGAAAACCCTTTTT
AAACNTTTTTCCCCCGNNGGNATTCCCTNAGNGCNNTTCNNGGGNCNNTNTAAAGGGGNTAAAANC
AAAAAANTTNCCNNTGGTCCGTTTCANAATTCCNNTNCCCNTNGGTNTATCAACCTNGGCCNCTNGG
NCGCTTTTGNTNTACATNNNNNCCCATTTCCTTTTTTNCCTCTTTAGNCCCCNNTTNTGCCCNCCCCCCT
TTGANNACT
TTNAACACAGCTGGAGCTCCACCGCGGTGGCGGCCGCTCTAGAACTAGTGGATCCCCGGGCTGCAGGA
ATTCGGCACGAGGCTCAGAACTAAACTGTTAGGGTGGGTTTTTGCGTGGAGGACAGGGGTAGTCTTTT
GATATTCATTTTTTGATTTTTGAGCTTTTTCTGCTCTAGGGTACGGGAGATGAACTTTATTTACAATACTT
TGATTTGGCAGGTTTTCTTCTACTTTTGGCTGCCTGNAACTGTTNCCATATGATGTAAAAAGCANGTGT
AGTGTCCCATTATTATGTGGTTCGGGGGATTTTCATTTGTTTTTAAAAANTAACCCATGTCAGCTGGGA
TTAGACTNCCTANAATCTATCAGTGGNAAANTTACATTGAAAAAATGCCTTTGGAAAATNAAATTTCA
NACAAAAAGTGCTTAAGAACTGGTATTTTTTCTAAGTGCTTCTATTTATACCAAAAAGCNTTAAAGTAA
CCCANTGCCCAGTACCATTCTTGAAAAATTCTTTTATTTAACCGACCNGTGCTTATTTNATTAAAACCA
GNTGATTCCTNTTAATTATTACNGGGCCGTAAGNTATAATTATTGGGGTNAAAATACCTTGNAATATN
GGACTTTCTGGCCAGTNGGGGAATTTTCACTNAATTTCTTTTGGTGGNGGGATGGTTNGAATTAAAAN
CTGGAAGGNGGGGGAAAATTTGACTNNTTTAAAAGNGTGGNCCCCCCCAAAAATCAANAATTAAAAT
TGGGCCTGGGCCTGGNAAAACCTAATCCTTACCTNCNNAAATCTTAACCCCTTNTTAGGCTTGGAAAN
AAAAAGTTGGGGANTTTTGGAAAANGGTTGGCANACCTTTCCTTGGCCNAAAAAACCNTTNTTTTTGG
AAACCNTGGGCGGGANTTGGNTTGGGANTTTGNAAAAAAAAAATTTCAAATTGGTCCCCCCAAACTTN
TNNNNAAAAGCTGGAGCTCCACCGCGGGGGCGGCCGCTCTAGAACTAGTGGATCCCCCGGGCTGCAG
GNNTTNCAGGACNGCTNNAGGGAGAGGAAGCNGGGTGTCTCCNANNAGTNCNNGGNTGCANAGGGG
AGAGAGCTGACCCTTNTCAAACCCCTGCTTTGGGCCCAGCCCTGCGCCAGGTGCTTCACAGCTCTTATN
TGCCAGATTTTCCCACCTTGNGGCNCATGGNGCCCCTAACCAACATNNCAAGAGGCCGATCAGCTTGC
CACATNTGTGCANGACCTGGGACCACAGGCGTGGACAGGAAGGGCTCTTAAGCCNTCTTTTTACANAT
GAAAATGCCAGGGCCCAGGGAGGATNAGCAGTCTTTGGAGGTTACCCANCCGGAATACTGGGAGACC
TGAACCCAGCATTCTGACTCTGCATTNAGTGCTCGCTCCATGATGCAGNGGGTCCTAGCCCCCCNCTGA
AATGGGTCCCCTCTTCACTGNGTTTCCCAGNATNCTTGCTCTGACCAAACCGGNGAAGGGACACACNG
CCACCCNCAAAAGATGGAATCATTCTCTTAACCGTCTTTGTCCCAACTGCCTGCCNNTCCAAACCCCGTN
CCCCTGNNCCCTGACCNTGCTGGNTGGGGAAAGAGGCTCACGTGGGCCCAGGCTGAGCANNNAATGA
CCATGTCAAGCTGTCAGACTCTGNGAAANTANTGCATGCTACNGGCAACCCGGCNTCCCTGATGNCCG
CNTGATTTTGGACNTAANGNGGACATGAAAATGCCACAATTGGGGGNATGGGAGGGCCNCCCCCCCC
CCNTGGGTGTATGNCNATGGNCANGGGGTTTNGGTAACCTTTTTGGTTTTNGGGAATTTGAAANGCCC
AAAAANAACCCGGGTTGNTTTCTTTTGNAAAAAAAAAAAAAAAAAAAAATTTANNGGGGGGGGGCCG
GNCCCCGAT
GGAAACCAGTTGGAGCTCCCCGCGGGGGCGGCCGCTCTAGAACTAGTGGATCCCCCGGGCTGCAG
GCAGGATCNAGNGAGNCCAGGTTGCANNTTTTNNNNAANGANGATNNANNNNGGNATGGNTTCAAA
AGANAACTGGCANTACNATCTGGTACAACTCCANGAGAAGACATTGGAGAAGAACCAAGCTGGGTCT
ATAAGAAGCAAGCTGGGTCTATGAGAAGTGNNCNTAACGNGTATNNNATNTTNNAAGACAACCATAT
CACAATGAAACCNTGACTANTGTAANGCTNGNTCCATGANGNTNTNTNNTCACTATCAGNCTGAGACC
NAGGAATANATATAAAACGNTGCAAAAAAAAAAAAAAAANATNGGGGGGGGGCCCGNNTCNNANTC
GAACNTNGGGGGTGATGGNNTAAAGGAAAACNNTTTCNTANATNGTTGNTCTTCNANNGTTTTTAAAC
TNTTGANNATATTNCTGCNNTTTACTTTCNGAATTNGAANCTNANTTTTTGCTTNTNTTGGCCNCAA
NCACNTNNNNTTTTCNTTTAAGTGGNTNTTGTTTTNTTANAAAAANNTTCGANNCNCATTTTGGNTGGN
NTTTTTTCTNNCNTNTCCNCTGACTTGTCCNNNANATTNTANATCCTNNTTTTTNTNNNTNNNGTTGGA
TCTTTNNCCTTNTNNNNCGNNTTNNTGGGGGNNTTTCTNTTTCCCTANTTANTNCCTTTNNNTACNNGG
NTTTCTCNNTNNNNTTTNTTNCNAANNNTNNNCTNTTTATTNANTNTNTTANTTTCNNCTNTTGGNTCT
NTTTCTCTNNTNNNNTTTNTTNCNAANNNTNNNCTNTTTATTNANTNTNTTANTTTCNNCTNTTGGNTCT
TCTTTTNNNTNNTTTAGNTNTTCTNANNTCTNGTACTTCTTTNTTTNTNTNNANTNTTTACGCATTTTTN
TTNNCTTCNNATACNACNTAGANTCTCTNNTCTCCNCTNNC
GCCTATNTGGAGCNGCATNNCAGTGGCGGCCGCTCTAGAACTAGTGGATCCCCCGGGCTGCAGGAATT
CGGCACGAGGAAGTCGTGGTGATGGAGGGCCCCCTGGGGCTACTGGTTTCCCTGGTGCTGNTGGACGG
ACTGGTCCCCCTGGACCCTCTGGTATCTCTGGCCCCCCTGGCCCCCCTGGTCCTGCTGGTAAAGAAGGG
CTTCGTGGGCGTCGTGGTGACGAAGGTCCAGTTGGTCGAAGTGGAGAGACAGGTGCCTCTGGCCCTCC
TGGCTTTGTTGGTGAGAAGGGTCCCTCTGGAGAGCCTGGTACTGCTGGGCCTCGTGGAACCCCAGGTC
CACAAGGCCTTCTTGGTGCTGCTGGTTTTCTGGGTCTCCCAGGCTCTAGAGGTGAGCGTGGTCTACCAG
GTGTCGCTGGATCTGTGGGTGAACCTGGCCCCCTCGGCATCGCAGGCCCACCTGGGGCCCGTGGTCCC
CCTGGTAATGTCGGTAATCCTGGCGTCAATGGTGCTCCTGGTGAAGCCGGTCGTGACGGCAACCCTGG
GAATGACGGTCCCCCAGGCCGCGATGGTCAACCCGGACACAAGGGGGAGCGTTGGTTACCCCGGTAA
CGCAGGTCCTGTTGGTGCTTGCCGGTGCTCCTGGCCCTTCAAGCCCTGTNGGTCCCGTTGGTAAAACAC
CGGAAACCGTGGTTGAACCCGGGTCCTGCCNGNGCTTGTTGGTCCCTGCTTGGTGCCNTTGGCCCAAA
AGTCCCAGTGGCCCGCAAGGTATTCCGAGGNGAAAANGGAAAACCTTGGTGATAAAGGTCCCANAGG
TCTTTNCTGGCTTAAAAGGACACAATTGGGNGAAAGGNCTCCCGGNCTTTGCTGGCCATNAATNGN
NANCAANGNGGCTCCCCGGNCTGGNNNGTCCCCTTGNTCCCAANGGNCCCTTTTGGTTCCTTTTGGCC
CCTTNGCAAAAGAAGGTTNTATTTNNNCAANCTNGNGNNNTNAGCNCN
NCAAANCTGGACTCCACCGCGGTGGCGGCCGCTCTAGAACTAGTGGATCCCCCGGGCTGCAGGAATTC
GGCACGAGGAGAGGTGCAACTTTCTTCGGTCGTCCCGAATCCGGGTTCATCCGACACCAGCCGCCTCC
ACCATGCCGCCTAAGTTCGACCCCAACGAGATAAAATGTACCTGAGGTGCACCGGTGGGGAAGTCGGT
GCCACGTCTGCCCTGGCCCCCAAGATCGGGCCTCTGGGTCTGTCTCGAAAAAAGGTCGGTGATGACAT
CGCCAAGGCAACTGGTGATTGGAAGGGTCTGAGGATTACAGTGAAACTGACCATTCAGAACAGACAA
GCCCAGATTGAGGTGGTACCTTCTGCTTCTGCCCTGATCATCAAAGCCCTCAAGGAACCACCAAGGGA
CAGAAAGAAGCAGAAAAACATAAGCACAGTGGAAACATTACTTTTGATGAGATCGTCAACATTGCC
CGGCAGATGCGGCATCGGTCTCTAGCTAGAGAACTTTCTGGAACCATTAAAGAGATCCTGGGGACCGC
CCAGTCTGTGGGCTGCAATGTTGATGGCCCGCACCCTCATGACATCATTGATGATATCAACAGTGGCG
CAAGTGGAGTGCCCCCGCTAGTTAAAAACTGCAAANGAAAATAAAGGACCATTTTGACAACCAAAAA
AAAAAAAAAAAAACTTGGGGGGGGGGGCCCGGNCCCCAATTNCCCCCTTTANGGGGGGGNNNNNNN
NAAAANNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNC
NCCNNATCTGGAGCTNCACCGCNGTGGCGGCCGCTCTAGAACTAGTGGATCCCCCGGGCTGCAGGAAT
TCGGCACGAGGAGAGGTGCAACTTTCTTCGGTCGTCCCGAATCCGGGTTCATCCGACACCAGCCGCCT
TCCACCATGCCGTCTAAGTTCGACCCCAACGAGANNAAAATGGACCCTGNAGGGNCCCGGNGGGGAA
AGTCGGGCCCNGGTCTGGCCTTGNCCCCNAAAATCGGCCTTTGGNNTTGNTTCNAAAAAAGGCCGGG
GANNACTNCGCCAAGGNAANTNGGGNNTGGAAAGGGNCTGAGANTACNGNGAAACTGNCCATTCAA
AACAGACCAACCCANATTGNGGNGGNACCNTNTNGNTTTGGCCNTNANCNTCAANGCCNTAAGGGAC
CANCCANGGANCNNAANAANCCNNAAAACNTTANNCCNANGGAAACCNTTCTTTTNNTGNAAATNN
TAACATTTGCCNGGNGAANGCGGATTCGGTTTTATACTATAAAAAANTTTNTGGANAACCTTNAAAGA
GAATACCTGTGGGACACCCGCCCNCATTTNTGNGGCGTGTGNANNTGTTNTNGGGCGCGCCCNCCTCT
TNGANACNCNNTTTGNNTGNTATTTNNCACCAGTGNGCCCCCTGGCGAGNGNTCCCCCCTTCTNTTTTN
AAAAACATGNCGNCAGGNNGGAAATAAAAGNGNGNCCNTNTTTCNACCCCCCCNAAAAAAAAAAAA
AAATNTTNNCTNGGGGGGGGGGGCCCGGGNTTCCCCAANTNTTCCCCTTTTTTNGGGNGGGTTNNTNT
NTACNNCCNTTTTNTNCNCNNNATTTNATNTTTTNCTNNANNNAANTCTCCTCTCGCCNNTTCTTNGNN
AANTTNTTTCTNNNNTTCNTTNATNTTNCNTCTTTTTTTTNNNCTTACTCCCCTTTTTNNTTANTGNNGT
NCTTNNNTTTTTCTTNCTNTTTCCNNNNATNCAATATTNTTTANTNNTNTTCGATNCTNNNCGNTTCTCT
TNAATNTANTTATNTTGGTCC
TTNAGCCTATANNGAGNNGTATTACAGTGGCGGCCGCTCTAGAACTAGTGGATGCCCCGGGCTGCAGG
AAATTCGGCACGAGGNGATGACTTACCNNNTGGGAGAACANNGGNAGAGGGAATATCTTGNCNCCNT
TGGNTCCTNCACNTNCTTTGNAGGCCACCGGNNNTGGTACCTNTGACANCNGCNGNNTNCCNGGGNNT
AANCCNGGTACCAAAGGTTGNATNGCCCNTCTTNNANCANNTGTTCCCANANATCCCNTCTNATATCN
ACACANTANNGCCATCTGCCCAATTGAAGGCTNTATGCCTTNGAATNCCACNCTGACAAAAAATATTC
CCNANAGTAATATTTANCACNCNTAGANACGNGTGTGTNTCTNTGNCANTGNCTATANAANNTNGAGT
NGTGTNCACACACCCCNTGNTAGCGTGNNTNNTTTTTATAACCNNTTCTCTGNNGAGGNNTTTCTNCCT
TTCTCNACTCCACANCTTCTNCAATATCNCNNTGAGAGTGTNNGACNNTTTTCTCCACNGGANCACCCT
TGGATCTTTTATTCNTAAAAATNTCANCNCCCCNCATCANTTTTTCNANGAAGAANATGNNTTCCCCTT
CGTTGANGNTTNCCTTGNNTGNCNCCTCGTTNGTGGGAANGNTTNTCCCCCCTANNCAGTAANCCANT
NTTTTNNCNNAACANTNTTATCCCCCATTNTTTTNTTAGGGNGNGAAATTNNTTNNNACNAACTTCTTT
GTTGCCTCCAATAAANNNCTGNCNNGCNCCNTCATTNGTTTGGAAACAACCAGNCNTTTCTTTTCTNC
NGGGGNNTCNGNAAAAAACCACAANANANGTTACNTTATTTTNNACCCCNAAAGGTNCCCNACCCTG
GGCTNTNCGCAAAAAAAATNTTTTTTCTTNCNTAGCCNGNTTAATTNATTCANNTCCCCNCCCCNAGTT
TTTCCTNNNANNGANANAAANNNTTTNTGNNNTATGANNANNACNTTTTTTACC
TTNAGCCTATANNGAGNNGTATTACAGTGGCGGCCGCTCTAGAAGTAGTGGATCCCCCGGGCTGGAGG
AAATTCGGCACGAGGNGATGACTTACCNNNTGGGAGAACANNGGNAGAGGGAATATCTTGNCNCCNT
TGGNTCCTNCACNTNCTTTGNAGGCCACCGGNNNTGGTACCTNTGACANCNGCNGNNTNCCNGGGNNT
AANCCNGGTACCAAAGGTTCNATNGCCCNTCTTNNANCANNTGTTCCCANANATCCCNTCTNATATCN
ACACANTANNGCCATCTGCCCAATTGAAGGCTNTATGCCTTNGAATNCCACNCTGACAAAAAATATTC
CCNANAGTAATATTTANCACNCNTAGANACGNGTGTGTNTCTNTGNCANTGNCTATANAANNTNGAGT
NGTGTNCACACACCCCNTGNTAGCGTGNNTNNTTTTTATAACCNNTTCTCTGNNGAGGNNTTTCTNCCT
TTCTCNACTCCACANCTTCTNCAATATCNCNNTGAGAGTGTNNGACNNTTTTCTCCACNGGANCACCCT
TGGATCTTTTATTCNTAAAAATNTCANCNCCCCNCATCANTTTTTCNANGAAGAANATGNNTTCCCCTT
CGTTGANGNTTNCCTTGNNTGNCNCCTCGTTNGTGGGAANGNTTNTCCCCCCTANNCAGTAANCCANT
NTTTTNNCNNAACANTNTTATCCCCCATTNTTTTNTTAGGGNGNGAAATTNNTTNNNACNAACTTCTTT
GTTGCCTCCAATAAANNNCTGNCNNGCNCCNTCATTNGTTTGGAAACAACCAGNCNCTTTCTTTCTNC
NGGGGNNTCNGNAAAAAACCACAANANANGTTACNTTATTTTNNACCCCNAAAGGTNCCCNACCCTG
GGCTNTNCGCAAAAAAAATNTTTTTTCTTNCNTAGCCNGNTTAATTNATTCANNTCCCCNCCCCNAGTT
TTTCCTTNNNANNGANANAAANNNTTTNTGNNNTATGANNANNACNTTTTTTACC
TTTGANAACCGCTGGAGCTCCACCGCGGTGGCGGCCGCTCTAGAACTAGTGGATCCCCCGGGCTGCAGG
AATTCGGCACGAGGCTCTNTNCANGTNTGTANCTGNTNGCTCTGAGCNNTTGCCTGNNTTGTAGCANA
CTATGAAANCATACTNNGNCTCTCTANTATCCTAGNGNGAGTNTTACTCCTCCNGGNTTTTCAAAGGG
AGTCTTAAAGCAAATNATAGATTTTAAGTGAGAATTCAAGCAAATGTCNGATTAGACTAATCTTGCATT
NANATTTTAGATCTANGATATATATACACGCATGTGAACGTAGACACACCTGCATGTAGATATGTTTAC
AGGGCCTTAGCTATCTCAGTTNATTTTTACTTTGGACTTTNACCCATTTCCCCATTTACAGAATAAGTG
AATTTGTATAAGTTGGTAACNGCTCNCCATTTTTTTTTTTTTTTTTTNGGAAAAAAANAAGGGNGGGGN
CCNCNANTTTGGTAAAAAAAGNCTTTNCCCGGNGGCCAANATGGCCCTTANACTTTNGAATNTTTTTTT
GGNAAAAAATTTTTTTTAATANCNTTATTTTTTAANANCCCCTAAACCNCTTGGTTTGGGGAAACTTGNG
TTCCAAACCCCTGGGGGAGNAAATTAAAGAAAATNCCNNTTAACCTTGTTANNNAAAAAGAAGGGGG
TTTTNCCCCCNAACCCCTGTTGGTNTTCCCGGGNAAAAAAAAANAAAANTGGGAANCCGGGGTTTTCC
CCCCCGGGGCCCCGCCNCCTTAAAAANNNGGNACGGGGGATTTTTCCCCTTTTAAAACCNGGGGTTTTT
NAANCCCTGGGGNCCCCCTGGGNTTTTTTGGGGCCAAAAAAATTNCCCCGGGGGNAAAAAGGCAAAN
TTTTTTTTTTTTTTGGNAAAACCCTTNAACCCCCCCGGGGGGGGAAACCCCCNTTTCTTAAACCNTTTTT
TGGGAGGGGACCCCAAA
ANCANANCTGGAGCTCCCCGCGGTGGCGGCCGCTCTAGAACTAGTGGATCCCCGGGCTGCAGGAATTC
GGCACGAGGGANANNATAATGTGNTTCCTTATTTTTTAGATANGNNCTTAAGATAGGTTCATACTATC
CTTTGAAAAGAATTGACAACCTGCAAGAATGTNGTTCATACATATCATGGNAATTGCACAGTAAGGGA
GATTNTCATGTNGGATTTGGGGAACTANTTGCATTANTTCATAGCCATACTTGATGAATNAAGNCACA
NTGGCTGACNTTGATNCTGAATNAATGGACCTAAATANNTTTGTATTAATGNACTCTTATCTAAAACTA
ATTTACCATATTTGTTCTGTTTTTGAAAGTACAGGANTTGCCCTTTAATACTTGGTTCAAATTTGAGTAC
CTAAACCTTTCACACGAGTATAGATTAAATGGCCAGATACATTACTTTTAGAAAGTCGTTATTATTGATT
TATTTTAACCCCAATTGAGACAAATAAAATATTGGTTATTGAAAAATGNCTTTTCTTTCCATTACTGGC
ATCTCACATTAGCCCATCTGAGGGGAAAGACAAAACCCAGCTCATTTCACTGGTGGGAGTATTTCTCA
TTTTACATAAACTCCCATTATACTCTNCATCTCTTACGAACTTACAGAGNTAAAAGAGACATTTAGTCT
AACAAAGAACCTGGTTTTTTTCTGGCTCACAGATGGTTNCCTATGGNGGGATACCCGCCNTTTTATTTG
ACCTTTAGCCTTTTCTTTTTTTANACNCTGGGNAAATTTGNGAAATTCTAACCCTTTCTAAGCATGGTTA
GGTTAATTTATCCNCTTTTTTAAACTTAATTTTTTTAAGCCTTAACCTAAAAAAATTTTTTTTNNTTTAA
ACCTNAATAAAANGGGTTTTAANCTTTAAAAAAAANACCTTTAAAAANGGTTTTTTTC
TTGNNCACCGCTGGAGCTGCCCGCGGTGGCGGCCGCTCTAGAACTAGTGGATCCCCCGGGCTGCAGGG
AAGAACTCTGATGCAGAGGTTGCACCTTTTCCCAGATGAAGATATACCAGAAGATATTCTTAAGAATT
TAACAGAAGAGCTTCCTCAACCACGAAAAGTGCCCAGACGTCTAGATGAGTACACACAAGAAGAAAT
AGAGGCTTTCCCAAGAGTTTGGTCTCCACGTGAGGATTTATCGGCTGTAGGAGGAAGAAAATTTCAGAA
AATAACGGTGTGGTGCTTGAAACTCCTTCCTCAGGGCAGGATGAAGCAATAGCTGCCTTCTGGAAGGA
AGCCTGCTCTGGGGGCTGAAAGTTTGTGGGGAAAGGTCGGGAAGGGGTATCCCTTCATTAGGAAATTG
CAGTAAGATGTCATTTTATAGAATTGTTTTATTGTATAGTGCTATTTGAATGTTGGAAATGATAAGAAT
AAAACTGCGCCTTACTTCAAAAAAAAAAAAAAAAAAAAACNNTGGGGGGGGGCCCGGCCCCAATTCC
CCCTTATGGGNGGTGNCTTACACNCNNNNNNNNNCNANNACTGGGNNTGNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNTNNNNNNNNNNNNNNNNNNNNNNNNNNNNNNNNNNNNCCNNNNTNNNNNNANNNTNNN
NNNNNNNNNNCNNNNNNNNNNNNCNNGNTTNNNNNCCNNNNNNNNNNNNNNGGNNNNNNNNNNNG
NNNNNNNTNNNNNNNTTNNNNNNGNNNNNNTNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNN
GCCTATNNGGAGCCGNANCNCAGTGGCGGCCGCTCTAGAACTAGTGGATCCCCCGGGCTGCAGGAAT
TCGGCACGAGGATAGTTGAGACTTGTAGACATTTATTTATACTGTGTCATGTTTTATAATTTATACATA
AAATGTCTGGTTGACTGTACACCTTGTTTTTGAAGAAATTTATTCGTGAAAGGAAGAGCAGTTGTTATT
TATTGTGAGGTCTCTTGCTTGTAAAGTAAAAGCTTTTTTTTTTTCCTTGNAANCCNTTNAAGNCCNTTCC
TTACTATNCACTCNCTCATCNGNCTCCNTTCNTTNACCTGNCGTTANACTNTTTTCCACTTTANCCANCT
NGCATGTCAGTTNCTGNCCTGTTAATTNATNGNATNCTCNGCTGCCNGTTCTGNNCATNCCTGANCCCT
CGGGTTTNGTTNACAAGGAATCTNGACTGACCAAANGGCNTTATANCTNTGACTCAAATAGCAGGTCC
AGAGGATNCNTTTTTGAGGAANCNCCNATTTCTGTTCTCNGGTNGGNANNAAAACATCGGNGAGGGC
AGGNGGNNATANCAGGGTNGGGTTTCTAATCATGCCTTCTAANAGGTTNCANANCCAAANAATNGAG
NGATANGGGNGNCAGGACCCTGAAANAAGGAAAANGCTGTACATTCAACCTTATGTTAGGGGTTTCC
TTNAAAGGNTAGTTTGCTGTNCCTTTTCANCTAGTTTTTTTTCCCCANTGNGCCNTGAAAAGGTATATC
AAAGTTTNTNAGNCCANGGTTCTNGGCTTTTNAAAAAACTTTTTTNNNGAGGCAATTTTTTATTTNCA
AACATGGCAAGTAAAAAATAAANCNTTTAAAANNTNAAGTTTAANCTTTTTCTCCCCTGGAAAGGNCC
ANTNGTTAAAAAAAGTTAATAAGNGGGTTTTCGCAAAATAANGANGAGGGAAAAATTANAAAAATTT
TAAAANNCCNNAAAAAAAAAAAAAAAAAAANT
NAAAACTGGAGCTCCCCGCGGTGGCGGCCGCTCTAGAACTAGTGGATCCCCCGGGCTGCAGGAATTCG
GCACGAGGGGAAGACTTACCACGTGGGAGAACAGTGGCAGAAGGAATATCTTGGTGCCATTGCTCCT
GCACATGCTTTGGAGGCCAGCGGGGCTGGCGCTGTGACAACTGCCGCAGACCTGGGGCTGAACCCGGT
AACGAAGGCTCCACTGCCCACTCCTACAACCAGTATTCCCAGAGATACCATCAGAGAACAAACACTAA
TGTCAACTGCCCAATTGAGTGCTTCATGCCTTTGGATGTACAGGCTGACAGAGAAGATTCCCGAGAGT
AATATTTCCAACCCAGAGAAACAAGCGTGGATCTCTGCCAAGGTCCATCCAAACTGGAGTGGTGTTCG
CAGACCCCATGTTAGTGTGTTCCTTTCTTAAGCCCTTTGCTCTGGAGTAACTTCTCCAGCTTCAGCTCAA
CTCACAGCTTCTCCAAGCATCACCCTGGGAGTGTTTTCAGACTTTTCTCATACATGGGAGCAGTAGGTT
ATCTATTCTTCAAAATATTCCATGCCAATCAGTATTTTAAATGAAGTGATTTCTAACTTGNGATTTGGAT
TGGGATCCATAGGGAAGCACATGCCCCAACCAAGATCCAAAATGTCTTGAAATGATATTACCAAAATT
TTAAGTAGGAAAAGNTACCTGAACACTTNTGCTTNCACTTAACTGACTGGGCCCGCAATANTGNANGA
AACAGCATGNCCCTTTGNACTGGGGNATTCAAAAAAAAGCCCCAGTACTTCACTTTTTTCCAAANGAA
TCTAAGTAATTGGCCTAAAAAAAATCNTTTTTCNTAACCCGGGAATTTAATCAAATTTTTCCCAGNNTT
TTTTATTTGGGAAAAAAAAATTGGNNCTGGGAAAAANACCTTAANNNTGCCNGTTNNANAAAAAAGG
NAAACNGGGNCTAAAATAANGGGGGGGGGGAATAATTTTTTTAAACCCGG
NCNANATCTGGAGCTCCACCGCGGTGGCGGCCGCTCTAGAACTAGTGGATCCCCCGGGCTGCAGGGA
GAAGGTCTTCCTTGCCCAGAAGATGATGATTGGTCGGTGCAACCGAGCTGGGAAGCCCGTCATCTGTG
CCACACAGATGCTGGAGAGCATGATCAAGAAGCCTCGCCCTACCCGGGCGGAGGGCAGTGACGTGGC
CAATGCCGTCTTGGATGGAGCCGACTGCATCATGCTGTCCGGAGAGACGGNCAAAGGGGACTACCCCC
TGGAAGCTGTCCGCATGCAGCACCTGATTGCCCGNGAGGCANANGCTGCCGNCTACCATTTGCAATTG
NTCGAGGAGCTNCGTCGCCTGNCACCCATTACCANCGACCCCACCGAANCTGCCGCTGTGGGGGCCGT
GGAGGCGTCCTTNAAGTGCTGCAGTGGGGCCATAATCGTCCTCACCAAGTCTGGCAGGTCTGCACATC
AGGTGGCCAGATACCGCCCCCGGGCCCCCATCATTGCTGTGACTCGGAATCACCAGACAAGCTCGCCA
GGCCCACCTATACCGCGGCAATCTTCCCTGTGGTGTGTTAAGGGACCCANTGCANGAGGCCTGGGCTG
AGGACGTGGGATCTTNCGNGGTGANCCNTNNCCATNAATGTTGNGAAAAGGGCCCAGGGCTTTTTTNA
ANAAAAGGAGAAAATNGGTCANTTTTTGCTNAACCGGGTGGGCGCCCCTGGTTTTCGGCTTTTAACCA
ANACCCAATGCGGTGTAGTTTCCTTGTGCCCATGANGGGACTTCGAAGCCCCCTTTTTCCANGCCCCCT
NGTTNCCACCCCCTTTTTNCCCAAAAACANTTCCNTTAGGCCCAANNNTTGCTTTNGAATNGCTTCANC
TTTGGGGGCTNGAATNGGGGGCANCCTGGTGGGGGTTGGGANCACCCNANGGAAANAANAANGAAA
TAACCTTCTTGTGGAAANCCN
TTNNAGCCTATANNGAGNTGTATNACAGTGGCGGCCGCTCTAGAACTAGTGGATCGCCGGGGCTGGAG
GAAACTACACTTGGTTACCAGTTTGGGGTCGAGATGTCCTACGTCCCGGGCCAGCCGGTCACCGCCGT
GGTGCAAAGAGTTGAAATTCACAAGCTGCGTCAAGGTGANAACTTAATTNTGGGNTTNANCNTTGNAG
GCGGANTNGACCAGNATCCNTCCCAAANCCNTTTTTAAAAAAATAAAACANACAAGGNCNTTTACNT
ANNACGGNNNTCCAAAGGAGGCCNTGCGNAAATNGNTGGGNTGCAAATCGGGGACAAAGATCATGC
AGGTNAACGGCTGGNACATGACNATGGTCACACATGACCAGGCCNGGAAGCGGCTCACCANGCNCTC
NGAGGAGGTGGNGCGTCTNCTGGANACGCGNNNATCTCTGCAAANGGCCGNGCAACAGTCCATGCTG
TNCTANCANCTGCCAGGGGCCCCGGCTGTGCCTGCCCGCCNTTTGTACAGNGACACCANTTNCACGCT
NTGTCTGCCTCTCATCCTGGCTTCTGCTCATTGNTGGGCCCGGCNCTCAAAAGGGCAACACGGATGCCT
GCCCGGTCTGATCCCCTCTNCCCGTACTGCCCAGGTCTTGTGGCACCATNTCTCCCTCTNGGATGCTGA
AGACTGGCAACAGGGCCTGGACCCGANTCACAATCANNCTGNAGTGACAATTTTGGGCCNCAAAANC
TTGGTTGCTTGGCCAAAATTATTCCCCAGGTNATNAGGGGCCCTTTTTTCTTGATACCNCCCCCCNTNT
TAAAAAACCCCNGGGGNTTGGCCCCTTGCCCTTTTTTTTAAANTTCCGNAGTNNCCTGTGCCCCCACTT
NTTTTTCCTTTTTTTTCCCAAGAGGGGGACTTNTNTTCTTGGCAAAAAAAACT
TTNANCAACAGCTGGAGCTCCACCGCGGTGGCGGCCGCTCTAGAACTAGTGGATCCCCCGGGCTGCAG
GAATTCGGCACGAGGAAATGCTGAGGTGTAGACTGACAGCTTTAACACTGCTGAATGCCAGGTTACAC
AGTATTCTTACAAAAAGGGAAGTCAGCCAAAGACTGATCTGATGGACCAAAATTGGAATTTTGAAAA
GCACCAGTACTACTTTCCAAAATGATCAGCAGGTTAAACATGTTCAGCAAGGTATAGTGTGAATCCAT
TAACCCTTCGTCGTCCAGGGTCCAGACCAGAACTACTTGTTAGTTTTGAAAATGGTAGCCCAGAATGT
GGTTGTGTGCTTTATGGAAAGTCAGCAGTGGCTGCAGGCTTGGGTAAACAACCAAAGTTTTCANGGTT
GGNAAAACACTTCTTCCTTGTNCATATCCCCACCAGGCTTTAATTNAAAAATCTAAAGGNGGNCCGTN
ACCTNGGGTTNGGTTTGGTTTTNAATAAAGNCNTTNGAATCNGGTTTACCTNGNTTTTNAATATCTGNC
TTAAAGNAATCCTAAATCTTTTTAACCTNACCNTANTTGCANTTNCCCCTGNANTTTCATGGACCAAGT
TGGCTTTNANTTTGGTCAAGGGGGGGGTTTGGTTTTGGTTTTTTTTTTTGGCTTTTTAANTNAAAAACTT
TTGGTGGGCCAGGGAATCCCCTTTTTTCCCCTTTTTGGAAAGGNCCTTTTGGCCANCCCCCCTGGTTTA
ANGGGCCCTGGAAANCCCCTTACCCCGGCTTTGGAAGGGGTTATTTTTTTTAAANGGGGGCNCCCCAA
AATNCTTTGGNCCCCCCCTTTGGTTTTTAAANNNGGNAAAGGAACCTTGGAAANNNCNTTTTTTTANA
AAAATTTTTTTTTTTTTTTTAGGCCCCCNAANGGGTTNAAAAAANCCCCCAAAAANGGGGGGTNTGTN
CTNCCCC
NAAAAGCTGGAGCTCCCCGCGGTGGCGGCCGCTCTAGAACTAGTGGATCCCCGGGCTGCAGGAATTCG
GCACGAGGGTCTTTAAACCCTGCGTGGCAATCCCTGACGCACCGCCGTGATGCCCAGGGAAGACAGG
GCGACCTGGAAGTCCAACTACTTCCTTAAGATCATCCAACTTCTGGATGATTATCCAAAATGCTTCATT
GTGGGAGCAGACAACGTGGGCTCCAAGCAGATGCAGCAGATCCGCATGTCCCTCCGCGGGAAGGCTG
TGGTGCTGATGGGCAAGAACACGATGATGCGCAAGGCCATCCGAGGGCATCTGGAAAAGAACCCGGC
TCTGGAGAAACTGTTGCCTCACATCCGGGGGAATGTGGGCTTCGTGTTCACCAAGGAGGACCTCACTG
AAATCAGGGACATGCTGCTGGCCAACAAGGTGCCAGTTGCCGCCCGTGCTGGTGCCATANCGCCGTGT
GAANTCATTGTGCCAGCACANAACACTGGTCTGGGGCCCGAAAAANACCTCCTTNTTCCAGGCTTTAG
GCATCACCACNAANATNTCCANGGGGCACAATTGAAATCCTGATGATGTGCAGGTGATTAAAACAGG
AGACAAAGTTGGCGCCAGCGAAGCCCCCTTGNTGAACATGCTGAACATNTTCCCCTTTTTCTTTGGGCT
GGCATTCANCAGGGGTTNGACAATGGCAGCANTTTACAAACCTTGAAGTGGTTTGACAATNACAAAA
GGAAACTTTTGANTTCCCGNTTTCTGGGAGGGCGTNCCCNAATGTTTGNCAACCGTTTGCCTTCCAAATT
GGGTAACCCAANCCGTTGGGAATTTTGGANCCCCNTTTTTTTATTAAATGGGGTNCCAAGCCCCGGCC
CTGGGTTTTTGGCTTGGGGGNNAAATTGANTAACNCCNTTTCCNCNTTTNTTTAAAAAGGGGCAAGGG
CCT
TNNANAAAAGCTGGAGCTCCACCGCGGTGGCGGCCGCTCTAGAACTAGTGGATCCCCCGGGCTGCAG
GAATTTCGGCNCGAGGGANAGAGAGAGAGAGAGAGAGAGAGAGAGAGAGAGAGAGAGANAGAGNGG
GNGGNNCNGNCCCNNNCTNCGNTATANAGAGANNCATAANNCCCCCTNGCTACTNTGNTCCATATNT
AGANNACTGTANGNCCNNGGCACCCGCACACGGATCNTTTGTAGNATTCTTTCTTCGTAACCGTNNCG
AACCATNATNGGACCCATTCAGTTCNCTNACTCNTCTCCCTAANAATCTTCTGCTGNCGACCTAGCATG
NNCCACNANTAGGCCCCTNTNAGCGTACNNACTTCTCCCTACGTTATTCTTACTACTCCNCGGATANTC
TATCGTTGGAACCTTGGGGTTACGNACACGCNTGTACCGNGTNNCCTCTCTNNGNNACCGTGNTGAGC
CATGCGNGANCTTTNGTTTATCNNACCTTNTTTTGTTTACCTTTACCGTGANTTTGCTCATTTNTNTGAG
CCCTAANCTTCNTTGCGTNAATTATNNACATACCCGCCNNNANCNTTGTGGCNTGACCAAACTNCCTN
TGTTTGGNATNATCCGTAATAAATTACCGGACCGGCCTANTACTNNGCTCCTTTCCATGCGTTCCCCN
NACCGGANNNNCTTTTGNCNNTTNTNTNACCCNGAACCTAAATTGCTTGGCNTTTTTGTTTCCCGGGGG
GAAGGAGGAAAANCCCTTNTTCTTTTGNCCTGCACCTTTTTTTNNNNATTCNTTTCGGACAANCCCCGA
AAGTNTTTNNTTTCCAAAACCTTNTCTTGGGCCTAAGGNAATNAAATTTTTTAATATTCCCTNGGNCT
TGNTNAGTAAANGNGAAAAANTTTGNCCCCTTTTNGNNTATTTTTTGGGNAAAAAATTTGGGCCTTTG
AAATNNGAAACCCTTTNTTTGNNCNGGGNNCCAAGGGNAANGGGGNCCCCCCCTTTTTTTTNCNNT
TTNNCAANGCTGGAGCTCCACCGCGGTGGCGGCCGCTCTAGAACTAAGTGGATCCCCCGGGCTGCAG
GNCNNAGTGCGGTGCGCGCCTGCGCTGGCCCTCGCCTCGGAGCAGCCATGATGGAAGGCCTGGACGA
CGGCCCCGACTTCCTCTCTCAGGAGGACCGCGGACTTAAAGCAATAAATGTAGATCTTTCAGAGTGATG
CTGCTCTGCAGGTGGACATTTCTGATGCTCTGAGTGAGAGAGACAAAGTAAAATTCACTGTTCACACA
AAGAGTTCATTGCCAAATTTTAAACAGAATGAGTTTTCTGTTGTTCGACAACATGAGGAATTTATCTGG
CTTCACGATTCCTTCATTGAAAATGAAGACTATGCAGGTTACATTATCCCTCCAGCAGCACCAAGACCT
GATTTTGATGCCTCAAGGGAGAAACTACAGAAACTTGGAGAAGGAGAAGGGTCTATGACAAAGGAGG
AATTCACGAAGATGANNCAGGAATTGCAAGCTCAGTACTTTGGCAATATTCAAGANGACGGTTGGAAT
GCATGAAGTGTTTCTGTGTTTTAGNGGCTTGCACATCCCATTTTGAGGAAAGATTTAAATTTCCATGTC
TTCCTGGAATATAATCAAGATTTGAGTGNGCGAAGGAAAAAAAAAAAAAAAAAACCTNGGGGGGGG
GGCCCGGGCCCCCAATTNNCCCTTTTAGGGGGGGGNNTTNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNN
NNGCNGNNCNNCCNTNCTCNNNNNNNNNNNNNNANNNNNNNNCNCNCNNNNNTNCTCCCCCTNCNN
TTNATNANCNTCNCTNCNNGGGGNNNNNNGTANNGTCTNTNCNNTTTGAGCGNANTGGAGCTNCACN
CNGTGGCGGCCGCTCTAGAACTAGTGGATCCCCCGGGCTGCAGGNTNCCAGGGAANCNCNGGTTATTT
GNNTGTAAGNTCACCNNTGGGGANCAAGCTNANNTGGTGNNGANGGNCTGGTGCCTGGANCAAGTGN
TTNTTNTTCCACNTNNTTNCNTATAANCNTTTATNCCNNTCNNGGNCTGACCTCNCCAAGGGCCGGN
TGCATGCTGCTTGAGGATNAGTAGNATGTATGNAAATTNCAATNCTCTGTGACCACCCCTTGGTTCATG
AACCATCAAGACAGGCCGAGATTCNTAGACCTAATGCATTGANNTGNGNCGTGTNGATGCATANTGC
GACCTCCAGTGACACTTGNGCCTATTTGACGACANTGTGNGCGTGAAANCAGAGTAACTNAAGGCTGG
AGCNGGTCAGTGAGAGACATCCAAGTGCCCTNGGTGNANTAACCTTCGGGTAGNTGACNAANTCCGA
TCCTNCCACCNTACTANAGGGNCAAGTCCTTTNGNNGATTTTGGAATGAATGCNTGGTGGNANNNCCG
GGCNATTGNNGGANTGAGGGANGNCAATGNNTGANNGGGGTGNAAANNNAAACCCGAANGGGNAA
CACNAGCCTAGAGGGGGGNNCNNNAATNTTTTNGGGGGNAAACTTCAATGNNNCCAAAAAATTNAAC
CCCTGNAAANTTNNNGCTNCAAAAATTCTNTTANCAANTTNAANTTGTATTCNTANNAGGGTCTAGGN
CNNGNTTTTNNATNAAAANAANAANTTCGGGNNTGNGGTAACTTTTTGNTANAAAAANGAATGNTGC
GCCCANACCNNNGCTGGGATTNNTTTNNANGAAAAAAATANNNCCGGNGGNTTTCNTNNNTATGGTG
CGGCTTNNTTTTGCTATTNNNGANANTACTGNGGNAGNCTACAANGGNAANTTGACTCANCCANAACC
AGGGNCTCCCTTTTTNNTCCANTTNCGCCCCTGTNCAAAGTGCACNNGTANNNACATGNACNTTGGGN
TNGNGCGCGCATTTTCCCTTTTTNGNCNGGANAGGATTCTTTATCCCNCACTCCTANNGNGGTTGCAAA
AAACNCCTCNNATTTTTTTGCACATTTGNAGNGGGGGGAAGGAAANATAGTTNACCCTATTNTTGGAA
GNCGNTGCCNAAAGTNNTCACACANNACAANAGAGATNTTCTCTNTGTGNGGGGGNGTNGTCNCCNN
NNCTTACCTATNAATNGGCGCTNNTNNTTTNNNNNNANGNAGCCCNAACNCTATTANTTACATTCATC
GNTGNCTATNTCC
GNCNATCTGGAGCTCCACCGCGGTGGCGGCCGCTCTAGAACTAGTGGATCCCCCGGGCTGCAGGGAAT
TTCGGCACGAGGCGACCAGTGACTAGACTGAAAGAAACTCATCCTTTGGGAGTCATGGCAGCTGATGA
AGATAACAAAGAACAAGTTCTTAAGGAATGTGAGCAAGCTGAAGAAATTATGAAAGATAAACAAAAC
CAGAAATTATTAGTGAAATTACAAAGGAAAATATTCAGTTAAAGGAGGAGATCAAAAAACTTGAAG
CTGAGTTACAGGAGACCACCAGGACCTCCCAGATTAATGAGGATATTCCTGAAACAAAGATAAAGTTC
ACATCTGTGGAGAATCCTGAGAGTGACAGCGAGTTTTTAGATATCTCCTATTCATGTCAAGTGAGCTCA
AAAGTCCCTTATGAGCTGCAAAAAGGACAAGCACTTATTACCTTTGAAAAAGAAGAAGTTGCCCAAA
ATGTGATCAGAATGGAATACCATCATGTGCAGGTACAGAATGAAAATGTGATGCTCACGGCCAACCCA
GTTTCGTTAAATTCAGGAGTCAAATTCCAGGTTCACGTAGGAGTGTCTAAAATGAAAATCAACGTTAC
CGACATCCTGATGAACTGCTNNAAAGTCAGATGAGAGACAAGCTANAATTGAGTTTCTTTTAAGTCT
CCCAACGGAGGCGGANAAGTGGAGTACCGTGGAGTTTAACAAGCAGACCCNGAGCCGCCCTTTTTCCC
TTTTNGGGAAAGGGGAATTTCTTGNNAAGAATTTTTGAAAATGGAAAGACTNTCCTNTTTTTNTTAAAA
TCAAAACCTGGCCATAAAGGTTCCNGGTTTNTCCCTACNCCNAAAACNNCCCTTTGGAAAANGTTTTA
GGGTTTTTTCNNGGAGTTTTNTAAAAAGGCCANNGTTTTTGGACTNGGACNTTAAACCCCTTTNAAAA
CCNCCNGGTTNAAAAAAATTTTTAAANGGGTTTTTTTNTNCCCTTTTNNNGGGGGG
CAANANCTGGAGCTCCACCGCGGTGGCGGCCGCTCTAGAACTAGTGGATCCCCCGGGCTGCAGGCTCC
AGCAAAGAAGATAAAGCCTCAGACCCCCAACACATTTCCTAAAAGGAAGAAGGGAGAACGAAGGGC
ATCCTCCCCATTCCGAAGGATCAGGGAAGAGGAGATCGAGGTGGATGCTCGAGTGGCAGACAATTCCT
TTGATGCCAAGCGGGGTGCAGCTGGAGACTGGGGGGAGCGAGCCAATCAGGTTCTGAAGTTCACCAA
AGGCAAATCCTTCCGGCATGANAAAACCAAGAAGAAGCGGGGCAGCTACCGGGGAGGCTCCATCTNT
GTCCAAGTCAACTCTGTTAAGTTTGACAGCGAGTGACCATGGGCCGTCTATGGCACAGCAAGGGTGTG
CCCACTCAACGTCCTCAGTGGACCTGGGAATCCTAGGCTCTATCAGGGGAGGGTCTTGGTGAAGAGAG
CAGTTTANAATAGGTCTGAAGACTGCACTGTAACACCCTCTAAGGTCCTTTTTTGTGTTCGTGGTTTTG
TACAGATTTGTTTTTTGAGTGTTGGGTAGCAAGGACAAAGAGTGTTCATTTTTAAGAACAATTTTAGTT
GTCATTCCCCTCCATGTTCTGTGAAAGTCTTCATACTGAGAAATTTGGATATTTTCCTATTAAATCATTT
CTTATTGAAAAAAAAAAAAAAAAAAACCNTCGGGGGGGGGGCCCCGGGNCCCCAAATTTNNCCCNTT
TNNGGNNGGGGGNNNTTAANNNNNNNNNCNNNNNNNNTNNNNNNNNNNNNNCNNNNNNNNNNNNN
NNNNNNTNNNNNNNTNNNNNNNNNNNNNNNNNNNNNNNNNNNNNNNNNNNNNNNNNNNNTNNNNN
NNNNNNNNNNNNNNNNNNNNNNNNNNNNNNNNNNNNNNNCNNNNCNNNNNNTNNNTNNNNNNC
TTTGNAGGNCCNCNTAGNGGANGTTCGTCATTACAGTGGGNTGGACGNTTTANAACTAGGGGGCATCN
CCNCGGNCTTGGAANGTAAACTTACACCCNGTCTCCTTTNTTTTNNCGTCTATTATNNTTTNNATNNNC
NANNGGGGGCNTNTNATTNNTNNTNNTTCTANANATANCCTCGAAGCTTNACTCTTTTTNNNTCGNGA
CANNATNTNACNGGNCGGAAAGTANACTTNTATATNCNTGATGANCTCTTAACTTTTATNCNATGAGT
NGTTATNNTGTGTTNCTATNTNAAATTCATTANGCANNNANTCANATTANNTCTNCACTAACGCNTAN
ANCANTNAGTACCNCTGCGGGCTNCTNTTAANACTCGACCACGNGNGNAATNCNGNANAGTGNAGNN
TCNTNTTTGCTNAGAAAATTTTGCGTGNNGNCTTGNNAAGAATTCTGGGGNGACNAAGAATCANTNTCA
AGAGCTGAAATCGGTTTGCNNGACANTAGACTCANTGTGTTNACTNNNNTTGNANCTACNGTCCCNGG
NAANNNCGAGATTTNACACATNGGCTGCTTCGGNNAGGTAGGGTTGTNTTGTNGTTNTTGCNTTGGTT
NGAACNACAGGGCNNATTANGCNTTGGCNNTNAANTGNCCNTNNNAGACCNTTACCNATTANTTNTT
NTNTAAANTANNTATNNTNGAANGTTGNNTCNCCTNGATTCGNTTNACTTTGTGNCTGTATNCCTTCTN
TNACAAGNNNCNCTACCNTNNTTTTAAACNTNTTTGTGATNATGGGNTNNNTNCAATTNCTNNATTTN
TTTTTNCTTTATTTNGTTNGNNNNNNANTNANTANTGTNNATNATCNTGGANANTNNNTANNTNTGGA
GTTNANNTNGGTTTNNNNGTTCGTGATCNTNTCNTNATNNNNCNTCTTNNCNTNTATACNNNATNAGA
AGTNNANNATNTACACNNATCCTTCTTTCCTCCTCNTTTGGGAATNNCNTTGANGGTACTTNGTGAAA
AACANTNNGNNTTTTTGGTNAGNNCCCGNNGTTNNNAATNTCTANNCTCNATGNTTNNGTGNNNCTNA
GNTNNNNTANGAGGTANTGACAATAANNTAATTTGTNTCACNATTGGGANTNCNANTATGNNNTTTAT
NNTTTNTNNTNGGNNTTTNTTNTGNGTNTCCGTTANNTATNTACGTANNCNGAGTTTNTCNNCGTNGA
ATTCNNTCTGNTGNTANTATNANNGGGNTTATGGGNCTCCCTTGGNGCNNANTGGGTATANTCNGNA
AANNNTTGCCNGNTGNTGGGNATNTGTGTGCCGCCTAAGNGNCNTTTTNTAGGACTNCTCNATATTTN
NTNGCCTACNAAGGGTGTNACNTTGNGNTATTNNANTTGTGANTCTTATATGNTNANCANNNG
TTTGNAGGNCCNCNTAGNGGANGTTCGTCATTACAGTGGGNTGGACGNTTTANAACTAGGGGGCATCN
CCNCGGNCTTGGAANGTAAACTTACACCCNGTCTCCTTTNTTTTNNCGTCTATTATNNTTTNNATNNNC
NANNGGGGGCNTNTNATTNNTNNTNNTTCTANANATANCCTCGAAGCTTNACTCTTTTTNNNTCGNGA
CANNATNTNACNGGNCGGAAAGTANACTTNTATATNCNTGATGANCTCTTAACTTTTATNCNATGAGT
NGTTATNNTGTGTTNCTATNTNAAATTCATTANGCANNNANTCANATTANNTCTNCACTAACGCNTAN
ANCANTNAGTACCNCTGCGGGCTNCTNTTAANACTCGACCACGNGNGNAATNCNGNANAGTGNAGNN
TCNTNTTGCTNAGAAAATTTTGCGTGNNGNCTTGNNAAGAATTCTGGGGNGACNAAGAATCANTNTCA
AGAGCTGAAATCGGTTTGCNNGACANTAGACTCANTGTGTTNACTNNNNTTGNANCTACNGTCCCNGG
NAANNNCGAGATTTNACACATNGGCTGCTTCGGNNAGGTAGGGTTGTNTTGTNGTTNTTGCNTTGGTT
NGAACNACAGGGCNNATTANGCNTTGGCNNTNAANTGNCCNTNNNAGACCNTTACCNATTANTTNTT
NTNTAAANTANNTATNNTNGAANGTTGNNTCNCCTNGATTCGNTTNACTTTGTGNCTGTATNCCTTCTN
TNACAAGNNNNCNCTACCNTNNTTTTAAACNTNTTTGTGATNATGGGNNNNTNCAATTNCTNNATTTN
TTTTTNCTTTATTTNGTTNGNNNNNNANTNANTANTGTNNATNATCNTGGANANTNNNTANNTNTGGA
GTTNANNTNGGTTTNNNNGTTCGTGATCNTNTCNTNATNNCNTCTTNNCNTNTATACNNNNATNAGA
AGTNNANNATNTACACNNATCCTTCTTTCCTCCTCNTTTGGGAATNNCNTTGANGGTACTTNGTGAAA
AACANTNNGNNTTTTTGGTNAGNNCCCGNNGTTNNNAATNTCTANNCTCNATGNTTNNGTGNNNCTNA
GNTNNNNTANGAGGTANTGACAATAANNTAATTTGTNTCACNATTGGGANTNCNANTATGNNNTTTAT
NNTTTNTNNTNGGNNTTTNTTNTGNGTNTCCGTTANNTATNTAGGTANNCNGAGTTTNTCNNCGTNGA
ATTCNNTCTGNTGNTANTATNANNNGGGNTTATGGGNCTCCCTTGGNGCNNANTGGGTATANTCNGNA
AANNNTTGCCNGNTGNTGGGNATNTGTGTGCCGCCTAAGNGNCNTTTTNTACGACTNCTCNATATTTN
NTNGCCTACNAAGGGTGTNACNTTGNGNTATTNNANTTGTGANTCTTATATGNTNANCANNNG
TTNAAAAAAGCTGGAGCTCCACCGCGGTGGCGGCCGCTCTAGAACTAGTGGATCGCCCGGGCTGCAGG
AATTCGGCACGAGGTCCTNGTCGTNGCTGAACTTTCCGGAACCGCAGCTGCAGCCGCAATGGCCCCAC
CCTGGGTGCCTGCCGTGGGCTTCACGCTGTTGCCCAGTCTGGGGGGCTTCCTGGGNGCCCAGAACACC
CGAGGANANGGTTTCCANATGGTACGCCAGCCTGCAGAAGCCCCCGTGGCACCCGCCCCGCTGGATTC
TGGGTCCCATCTGGGGCACACTCTACTCGGCCATGGGGTATGGTTCCTACATGATCNNNAAAGAGCNG
NNGGGNTNTTTAANGGAAGCGNTGNTTNCNCTGGGCNTNTACGNTNNNCAANTGTCTCTGAACTGGGC
ATGGCCTCCCCTNTTCTTCGGCACTCGACAAATGGGCTTGGGCCTTGGTGGATCTCCTGCTGACTGGCG
GCATGGAAGCAGCCACGGCCAATGGCCTGGCACCANGTGAGCCCGNCGGNTGCNTGCCTGCTGNACC
NGNACCTGGCCTGGCTGNCCTTTGCGGCCATGCTCAACTACCGAATGGGGNAAGACAACCAAGTCCGG
AGGAATGGCCGGCGGNTTTTCGGAATGAAGAACCCCTTNCCCTTCCANGAATTGCAACCCGTTGNGGG
TCAAGGCACTTGTTGGGGGGGGGGNCCCCAAGGCTTTCCGAANNCACCAAGGCCCGCCTTGTTTTGNT
NTTAANCCCAGGGGGNGAACCANCAATTTCCAAAAGGGGCTTCCTTNTGAACTTGAACCCCCCAAGCC
CAAAAAAAAAGGGNNCTTGGGCCCTTTAANCCCTTGNTTNAAAANTTGGTTTTTNAAAAACATTGGAA
ATTTTNTTTAACCCAAAANAAAAGTGTTTTTAACCTTTAAAAAAAAAAAAAGGNAAAAATTTGGGGGG
GGGGGCCCCGGGNNCNCAAATTTTTCCT
AAAAGCTGGAGCTCCACCGCGGTGGCGGCCGCTGTAGAACTAGTGGATCCCCGGGCTGCAGGNANGA
NAACNNNGCCCTGCCCAAGAAACAAANGCANAATCGTCCCATTCCTCAATGGATTCGAATGAAAAC
TGGCAATAAAATCAGGTACAACTCCAAGAGAAGACATTGGAGAAGAACCAAGCTGGGTCTATAAGAA
GCATGCTGGGTCTATGAGAAGTGGTCTTAACATGTAGACCACTTTTTTAAGCAGCCAGATCACANTGA
AACATCACTACTGTAATGCTCGGCCCATGATGTTATTTCCTCCTNTCAGGCTGAGACCGAGCATTAATA
TAAACGGTNGCAAAAAAAAAAAAAAAACTNNGGGGGGGGGCNNGGACCCAGTTTNCCCCTNTGGGN
NGTTGAANNCANTTGCNAAAGAAGCCTCNATAANNTCCTTNAAGANTACCNGGNGTCCNTCANNGGG
GAACTTGGAGAACAAANANCNCAANGGGNNNTAGCNTTTTTGAACNGGGCTTNGCNAACNAATTAAN
CNCNTTCTTTGGTAATTTTAAAAACCANTNAATTTTTTTTNTTGGGGGAAAAACATTAATCCCCNTNGG
GTTGGGNTGGNNNTGNNTGGACTTANCGGGGAAGGGATTGGTGTAACCCCCATATTTTGAATTTTTNT
TTTAAGGGTTGGTTTCAAAAANAGGGAAAAAANGNGTTANCAAAANNTCNCCANNTTCCTTTTGGAN
AAAAANCNCCCTTNNCNNNCNTAANCNGGNCCNNGGGTAATGGTTTTTTTTATTCCCNCCNAAAACCC
AAGGNTTTTAAACNTTTTTGGNCCCTGGNGGGGNAANTCCCNNGNNCTTTTCTTTNNTTTTTNNANCNN
TNGNTTNAANTTNNGGACCNNGNGGGCTTTGGTTTTTTNAAAAAAAACTTNTNCTTTGGGNGGGTTNN
NTTTAAAAATTTAAACCCGGGTTTNAAANCCTTTTTTGGGAAAAAAAAAACNT
TNNNNAACAGCTGGAGCTCCACCGCGGTGGCGGCCGCTCTAGAACTAGTGGATCCGCCGGGCTGCAG
GAATTCGGCACGAGGGGAGAATTCAAGTGTGATCCTCATGAGGCCACATGCTATGACGATGGGAAGA
CTTACCACGTGGGAGAACAGTGGCAGAAGGAATATCTTGGTGCCATTTGCTCCTGCACATGCTTTGGA
GGCCAGCGGGGCTGGCGCTGTGACAAGTGCCGCAGACCTGGGGCTGAACCCGGTAACGAAGGCTCCA
CTGCCCACTCCTACAACCAGTATTCCCAGAGATACCATCAGAGAACAAACACTAATGTCAACTGCCCA
ATTGAGTGCTTCATGCCTTTGGATGTACAGGCTGACAGAGAAGATTCCCGAGAGTAATATTTCCAACC
CAGAGAAACAAGCGTGGATCTCTGCCAAGGTCCATCCAAACTGGAGTGGTGTTCGCAGACCCCATGTT
AGTGTGTTCCTTTCTTAAGCCCTTTGCTCTGGAGTAACTTCTCCAGCTTCAGCTCAACTCACAGCTTCTC
CAAGCATCACCCTGGGAGTGTTTTCAGACTTTTCTCATACATGGGAGCAGTAGGTTATCTATTCTTCAA
AATATTCCATGCCAATCAGTAATTTTAAAATGAAGTGATTCTAACTTGNGATTTGGATTGGGATCCATA
GGGAAAGCACATGCNCCAACCAAGATAGAAAATGTCTTGGAAATGATATTACCCAAAATTTTAAGTAG
GAAAGTTACCCTGGAACACNTTNTGNNTTTCACNTTAACTGGACTGGGCCCCGCAATATTGNANGGAA
CCAGCATGGCCCTTTTGGACCTGGGGGAATTCAAAAAACAAGCCCCAGGNACTTCCNTTTTTTTCCAA
AAGGAAATCTAAGGAATTGGCCTAAAAAAAAAATTTTTTTTNNTTACCCGGTAATTTAAANAAATTT
TTNCCCAANNTTTTTN
TTNAACAANNCTGGAGCTCCCCGCGGTGGCGGCCGCTCTAGAACTAGTGGATCCCCCGGGCTGCAGGT
TTGATCCCTGGGTCGGNAAGGTCCCCTGGAGAAGGAAATGGCAACCCACTCCAGTATTCATGCCTGGA
AAATCCCATGGACCGAGGAGCCTGGTGGGCTACAGTCCATGGGATCACAAAGAGTCGGACATGACTG
AGCGACTTCATTTCATTCATTCAAGCCGACCTTTGTTCCCCTTTGAAATCCCTTCAAGCTTTTGGATGTT
CTTGGTGGTAACAGCATTAGATTTTGGGATATCTTTCTCGGATTCAACCTTGCATTTAATGGTTTTTAGA
CATTAACAAAACGGATTCTTTAAAAGGAAAGAAATTTAAGTCTACCTAATTCTGCTAGGCACTCGTGGT
CCCTGTGTCAACCGTACTTTTATAACTATGGTAATGAAGTCAATAGATAGCAAACTAATTATTCCTTCC
CCCTTGTAAAATTGAGAAAACTTGCTAACAAGGAACTGAAACTGTTATCTTGAACATAGTCCCCAGTC
TAGTTTTTGCCATGACTATAATGAATATAGTCATGTGGNTAATTTCAATTTCCTTGNGGTTAATTTCAA
AAGAATTCTCAGATCCCACAGTATAAANGGTGGAAAATTATGGTACCTTTTTATGCTTGANAAAANGAA
ANTNGGGGGGTTAAACCCATTTNAAAGNNNCATGNAATCATGGTTTNGGAAAAGGCATTTGGANTNCN
TTTGGAGTTATATATAGGAACCGGCAAGTTTTTGGAGTTGAATCCTTTTAAACATGCCGGACANAATA
AAAAACCTTNGTTTTGNNAAAANNNGGNCCCCCCANTTTNNTNAAANGGNAAANNTTTTNNCCNTTTGG
CCTTTTNAAAACCAGNNNNCCCCA
NCAAAANCTGGAGCTCCACCGCGGTGGCGGCCGCTCTAGAACTAGTGGATCCCCCGGGCTGCAGGCG
ACAGATAGCCGACTTCTGTGCTGTGCCAGCCGCATCCCTGAGACAAGATGGTGAAGGTCGGAGTGAAC
GGATTCGGCCGCATCGGGCGCCTGGTCACCAGGGCTGCTTTTAATTCTGGCAAAGTGGACATCGTCGC
CATCAATGACCCCTTCATTGACCTTCACTACATGGTCTACATGTTCCAGTATGATTCCACCCACGGCAA
GTTCAACGGCACAGTCAAGGCAGAGAACGGGAAGCTCGTCATCAATGGAAAGGCCATCACCATCTTC
CAGGAGCGAGATCCTGCCAACATCAAGTGGGGTGATGCTGGTGCTGAGTATGTGGTGGAGTCCACTGG
GGTCTTCACTACCATGGAGAAGGCTGGGGCTCACTTGAAGGGTGGCGCCAAGAGGGTCATCATCTCTG
CACCTTCTGCCGATGCCCCCATGTTTGTGATGGGCCGTGAACCACGAGAAGTATAACAACACCCTCAA
GATTGTCAGCAATGCCTCCTGCACCACCAACTGCTTGGCCCCCCTGGCCAAGGTCATCCATGACCACTT
TGGCATCGTGGAGGGACTTATGACCACTGTCCACGCCATCACTGCCACCCAGAAGACTGTGGATGGCC
CCTTCCGGGAAGCTGTGGCGTGACGGCCGANGGGCTGCCCANAATATCATCCCTGCTTTCTACTGGCG
CTTGCCCAAGGCCGTGGGGCAAGGTCATNCCTGAGCTCAACGGGAAGCTTTACCTGGCATGGGCNTTCC
GGGTCCCACTCCCNACGTGTTTGTTGNGGATCTGACCTTGCCGCCTGGGANAAACCTTGCCAGTNTGAT
GANANCAAAAAAGGNGGNGAAAACAGGCGTCAAAGGGCCCTTTCAAGGGCATTNTAGGCTTCNCTGG
AGGANCAGGTTGTTTCTTGNAAATTNAAAAGNGACACNTCCTTT
ANCAANNNGTGGAGCTCCACCGCGGTGGCGGCCGCTCTAGAACTAGTGGATCCCCCGGGCTGCAGGC
AAACTCCACCTGGACTACATCGGGCCTTGCAAATACATCCCCCCCTGCCTGGACTCGGAGCTGACTGA
ATTCCCCTTGCGCATGCGGGACTGGCTTAAGAACGTCCTGGTCACGCTGTACGAGAGGGACGAGGACA
AGAAGCTCCTGACCGAGAAGCAGAAGCTGCGAGTGAAAAAGATCCACGAGAATGAGAAGCGCCTGGA
GGCTGGCGACCATCCTGTGGAACTGCTGGCCCGGGACTTCGAGAAGAACTACAACATGTACATCTTCC
CTGTGCACTGGCAGTTCGGGCAGCTGGATCAGCACCCCATTGACGGGTACCTGTCTCACACCGAGCTG
GCCCCACTGCGCGCCCCCCTTATCCCCATGGAACACTGCACCACCCGCTTTTTCGAGACCTGTGACCTG
GACAACGACAAGTACATCGCCCTGGACGAAGTGGGCCGGCTGCTTCGGCATCAAGGAGAAGGACATC
GACAAGGACCTCGTGATCTAAAAGCCATGCCTCCTCCTGCAGAACCAGATTCTCTCTCTTTGACTTTCC
CCTTCTGTTTTTCCCAATGTTTAAAATGGTTGGATGGGTTGTTGGTCTTGCCTGGGGTCAAGGGGCTAA
TATAGACTTAAACGAATACCNTTAACCGGGGCTNAACACAGAACTTTTTAANCCAAGTCNCGAAATTN
NTTAAGTGNNAACTNAACTTTTTNCGGGCTTTTTGGTTACCCATGAAANGGGCCCCGTTTTTCTTTTTG
GCCGGGGNGCCCCTTCCNCCCTTTGGNTTTNGGGGGGCNAATTTGGGNGNGGAACACTTTNNNTTTGGT
TNNNGNTTTNNCCNTTAAAANACCAATTGGGNATTTTTNNAANATTTTNAATNTTTTTTNNTNNAAAA
ACTAATTTTNCCCCANNNCCNAAANTTTGGGTTTANTTTTTTTNNNGGGGGNNTGGGCG
GCCCTATANGGAGNNGNATNACAGTGGCGGCCGCTCTAGAACTAGTGGATCCCCCGGGCTGCAGGAA
TTCGGNCACGAGGNTGNAATGTNCTNTNCNGACTGNNNGNCNCNCNCANGATANNNANGGNAGAAGCN
NACCTNGTGGNGCTTTGGGCCCNNCTGGGGACAANNTCTAGCCCATNCNTCACTCTATTGNNACTCTT
NCAGGGCCTGGAANCCCTTGNTCCTATNTTCTNCTNGNGCTAGGGGCCTGNGCTTNNCNANNCCGNCN
GGAAAANGGANNGATNNACCCAGGGNGANNNCNGGAAATCAAANCTNANTGGAAAANCNTNGCCCN
NNNCCCAGGNGNGNAATTNNGCCTGNANACTGGNNGACCATGATGACNNTGAGGNTGTGGTNGNNAG
CCACNAGGGCCTTCANNAGNATGNAGNNGNGTTGGTTTTACNCGANTGCCNGGGCTACCAGTTTTTTC
AAATATGTNNNTTGNTGCCGTNCCCCCCNCCCTTNNNNTNGGTNNATANANNNNCTTNTGTNCNACNN
CNACCNTNCACANAGAGATTAACNNAAGNCTTANTGTCCCNANAAAANTGNTTAAANCNCCCNGGAG
CAAGGGTNCNTNCNNATNGACNATTTTTTCCCNATTTNGGNAANGGTTCTTATANATCCNGNTGNTNC
CCTTCCTTCATCTTNGNCATNCCAGATANTACNTGGTGNGATGGGNCCCCCNTCTTNNCANNTATNNCT
NNTTTNGCANACCANGATCNGTNNNGNANTNNTCCAAAAACANNCATTCCCTTNATTNNANTATTNT
TNCTNGCNNAANATCNNNTNTNTNNAATNGNNNATTCNTCNTTTACCTTNNANNNGGNTAATTATANN
TTGGGCNATGTGTTTCTTNCCTGNCGACCNTNCAACNTNNCTGTAATNATTNTTTANTTNGGTANTNTT
ACATCCNNCANNTNTNTTAATANNATNCNNTGTCCNANTTNATTAATNNNTATTNNNACTAAANTATN
CNNTCAATTCNTNCNTCCG
TTNAGNCCTANNNTGNNTCNNTNTNNAAANGNANNACNCTNTAGAACTAGTGGATCCCCCGGGCTGN
AGGCAANCNGACACNAGGNNTCTGGGNCANTAGCAAAAGTATCCCTNTTTAGGGGNATNTNNATG
GTNNGANTNGAANNGGTTNAAACGATNNGAACGNCTTGNTCTAAAAGGCTNGGNNNCANNNNCCG
GAAAANNGACANANCCTANCTNTTTTGNNTNGCTTAANCNACNTTTNAANCTAAGNCNNGNCNN
NTTNANNTNCNCNCNAGGGNATNGNGNCANNNANNGTNCCNANAGNAANAGATCGGCATTTNNNTAT
ANCNGNGNGANNGCGTCNATCCTATTTTTTGTGNNGGGCNAAATGGNGTGACAANNTAGNNGGGCN
NATGAANGGCCTCATTNATANNTNGGNANTCACAACAGGGGTANNTTTTNGTNNTNNGAANCTNG
NTNGCCCATNNTTGNNAAGNGTNTGCTCCAAAANANTTATAAAANNTNNTNACNACGCNCTTTGNGC
GAGNGTCCCCCCTTANTTNTNTNGTGTCNGGNNCTNTGCAACNCNCNNAGTAANTAANNAAGANCTC
TCATNNNTTAGNNNNCNANATTGNCATATNNTNCTTNTNATCCCTAATCTCTATGNANNNANCCANN
NTNCTCANNATTNTTTCCAACAATTCCNCTTTTTNTCNNTTANCTCNTCGNNANCCATNTNTNCNANAA
GTAGTCGNNGATNNNATTATNCCGCNCTNNGNNGATNNTAGTTGNGTTNNTCNNNNTNNNTATTTNG
AANCNNTCNTTCATTTGACTCGCCTNNATNTNNTNTAGCCTTACTTANGAACACNTNAACTTTCTTNAC
TNTATTCTCNTNTTTTNCGNCNATGGCTGCTCNAAANACCNTNTCTTTCTCNAATGACANNANTCGGNN
NCANATTTNTATTAGGTNNNTTACNCCTNGTNNCNCCTTTTTAATCTNTTTNTCNNNNTNNANNNNCTN
GGATCGACNCTCGCTTTNNNNTGNCAAATCCCNCTANTTTNNNCCCANATCCNCGCNCTNNNNCANAN
NGTTNATNNNTATTNAGANNTCCCNNNACATNTNTNCNNCG
TTNAGNCCTANNNTGNNTCNNTNTNNAAANGNANNACNCTNTAGAAGTAGTGGATCCCCCGGGCTGN
AGGCAANCNGACACNAGGNNNTTCTGGGNCANTAGCAAAAGTATCCCTNTTTAGGGGNATNTNNATG
GTNNGANTNGAANNGGTTNAAAACGATNNGAACGNCTTGNTCTAAAAGGCTNGGNNNCANNNNCCG
GAAAANNGACANANCCTANCTNTTTTGNNTNGCTTAANCNACNTNNTTTNAANCTAAGNCNNGNCNN
NTTNANNTNCNCNCNAGGGNATNGNGNCANNNANNGTNCCNANAGNAANAGATCGGCATTTNNNTAT
ANCNGNGNGANNGCGTCNATCCTATTTTTTGTGNNGGGCNAAATGGNGTGACAANNTAGNNNGGGCN
NATGAANGGCCTCATTNATANNTNGGNANTCACAACAGGGGTANNTTTTNTNGTNNTNNGAANCTNG
NTNGCCCATNNTTGNNAAGNGTNTGCTCCAAAANANTTATAAAANNTNNTNACNACGCNCTTTGNGC
GAGNGTCCCCCCTTANTTNTNTNGTGTCNGGNNCTNTGCAACNCNCNNAAGTAANTAANNAAGANCTC
TCATNNNTTAGNNNNCNANATTGNCATATNNTNNCTTNTNATCCCTAATCTCTATGNANNNANCCANN
NTNCTCANNATTNTTTCCAACAATTCCNCTTTTTNTCNNTTANCTCNTCGNNANCCATNTNTNCNANAA
GTAGTCGNNGATNTNATTATTNCCGCNCTNNGNNGATNNTAGTTGNGTTNNTCNNNNTNNNTATTTNG
AANCNNTCNTTCATTTGACTCGCCTNNATNTNNTNTAGCCTTACTTANGAACACNTNAACTTTCTTNAC
TNTATTCTCNTNTTTTNCGNCNATGGCTGCTCNAAANACCNTNTCTTTCTCNAATGACANNANTCGGNN
NCANATTTNTATTAGGTNNNTTACNCCTNGTNNCNCCTTTTTAATCTNTTTNTCNNNNTNNANNNNCTN
GGATCGACNCTCGCTTTNNNNTGNCAAATCCCNCTANTTTNNNCCCANATCCNCGCNCTNNNNCANAN
NGTTNATNNNTATTNAGANNTCCCNNNACATNTNTNCNNCG
TNNNNAAAGCTGGAGCTCCACCGCGGTGGCGGCCGCTCTAGAACTAGTGGATCCCCCGGGCTGCAGG
AATTCGGCACGAGGCCCCTCCCCCCGAGCGCCGCTCTGGCCGCACTGCGCTCGCCCTGAGCTCCGGGC
TCCTGCTAAGCCAGCGCCGCTGTCGCCTCCCTCCAGTCGCCATCATGATCATCTACCGGGACCTCATTA
GCCATGACGAGATGTTCTCCGACATCTACAAGATCCGGGAGGTCGCGGACGGGCTGTGTCTGGAGGTG
GAGGGGAAGATGGTCAGTAGGACAGAGGGTAACATCGATGACTCGCTCATTGGTGGAAATGCCTCCG
CTGAAGGCCCCGAGGGCGAAGGTACCGAAAGCACAGTAATCACTGGGTGTCGATATTGTCATGAACC
ATCACTTTGCAGGAAACCAGCTTTCACAAAAGAAGCCTACAAGAAGTACATCAAAGATTACATGAAGT
CAATCAAAGGGAACTTTGAAGAACAGAGACCAGAAAGAGTAAAACCTTTATGACAGGGGCTTGCANA
ACAAAATCAAGCCCATCCTTGTTAATTTCAAAAACTATCAGTTCTTTATTGGTGAAAAACATGAATCCA
GATGGCATGGTTGCTCTGCTGGACTACCGNGAGGATGGNGTAACCCCATATATGAATTTTNTTTAAGG
ATGGGTTAAAAAATGGGAAAAATGTTAACCAAAGTTGGCAAGTTACNTTTGGAATCAAATCACCTTGT
CGGCATAAACTGGCTNGGCTACTGGGTTTTCATTCCCCCCCACCACCAGGACTTTAAACAGAANGGGA
ACTGGATGTCATTCTCGAGCTTTTTANTTTGGTTTGAACCGTTGANTTAATTTGGAGCGGGNGGCCTTT
GTTTTTTGANAAAAACGGGTCATGGGAGGGTGGCTTAAAAAANAAAACNCCTTTTAAACTTCNTTGGN
AAAAAAAAAAAAAAA
AANAGCTGGAGCTCCACCGCGGTGGCGGCCGCTCTAGAACTAGTGGATNCCCGGGCTGCAGGNANGA
TCNANCNNGTCCTGCCCAANAAACAAANGCNTAATCGGCCCATNGGTTAATGGATTCGNNATGAAAA
CTGCCAGNTATAATCAGGTACAACTNCANGAGAAGACATTGGATAAAGAACCAAGCTGNNCTATNAT
AAGCANGCTGGGTCTATGANANGTGGCCTTAACATGNAGACCACTTTTTTTAAGCANNCANATGACAT
TGAAAACNTCACTTACTGNNNTGCNTCGGCCCATGANGTTATTTCCTTACTATNAGGNTTGAAACCCAT
CCAATAAATNTAAANCCNNGGCAAAAAAAAAAAAAAAAANTCCNNGGGGGNCCCCTTNNCNCANANA
NTCCCTTAAAGTAAAATTGTTTANANAGANCANTCNATCTTGNNGCTTTTNTTTNCCNCNTNNGCAATN
CATNCANGNNTNNCCCCCNTTGGNGGNNTTTTTNCNTTTATANNTCNTNCNNNCTTNGGGGCTNNTCT
CGNANTTNCTNTNNCNTATNCCANTNNTTNTCCNTNAGNGTAAAAAANCATCCNTNTAAANCCNNAT
TANAAANNCACACTNNGNNANCANTTTATACNNNTCNTCACTCGGAANGAANNCTTNTTATTTTNTT
TTAACGNCAATATTNNNNTTNNNTCCCTCTATTCATNGGTTAGGANTCTTATTATAATTCTCTNTTNNG
TNCCNNCNGAGANNNACTATTNTNNNTCTCAANTCNNNCTTCTTANNCATNTGTTCNNNNCNNCTATT
TTTTNNTTCNGANGGANNCTTNNCNNCTCCTNNATTNTNTCTTAAATTNNCGNNNTNATTCTTATTTNC
TCGCTNNCANACNNNCNNAGAGACTANCTTTTATCTTTATTCNTTTCTCNNTCATTANAANNCTTCATT
TTTCTNTTNCACTTCATTANTTNGTTGNGNNTCTTCNCTCTCTTTTTACNNATGTATTCTTNANTNTNN
GNCTGNTTTNTCTACGNTNGAACNTCTCCNNCNTCCCNCG
TTGNANACAATGTGGAGCTCCACCGCGGTGGCGGCCGCTCTAGAACTAGTGGATCCCCCGGGCTGCAG
GAATTCGATATCAAGCTTNTCNNTACCGTNGACCTGGANGGGGGGCCCGGGTCNCAATTCGNCCTATA
GNGNNNCGGANTACAGTGGGAACGGCGTCACAATGATGACTGGACGGCAGGGCCGAGCTACCTTCCA
GTTCCTGCCTGATGAGGCCCGGAGCCTGCCGCCGCCGAAACTGACGGACCCGCGGCTGGCCTTCGTAG
GCTTCTTGGGCTACTGCTCCGGCCTGATTGATAACGCGATCCGGCGGAGGCGGGTGCTGTTGGCCGGTT
TGCATCGCCAGCTTCTATATATTACTTCCTTTGTTTTTTGTTGGATATTATTTTTTAAAAACGTCAAGAC
TATATGTATGCTTGTGAGGGACCACGATATGTTCTCATATATAAAGTCACCATNCAGAGGATTTTCCCT
GAAAAAAGATAAGAAAAACTTACGGGGAAGNTTTTTGAAGAAATTCCATCCAGGGGCGTTGAAGTTC
TTNCAAAATGGTTTTGCCTCCCGGTTTAATTGATACAAGTTAATTTTCTGNAATTTTGGTGGAACTGG
TTTCTTATGAACATCTTNAAAGTTTTATGTTTNANTTTTCTTTAACCGTTTAAACCCCCTTGNTGATTAA
AAATTGGTCNATTNTAAAAAAAAAAAAAAAAAAAAAACNTNTNNGGGGGGGGGGCCCNCNCNNCCC
CNNTNTTTTTCCTNTTNTTTGGGANGCTCCNNNNNATCCNCTTNNTTTTCCCTCNNNNTTTCTNTACNTC
TTNCTNTTTCTTNCNANCCNCNATNCNTCTTTGCTNTNTNCTNCNTACTCTTTTNTTTNTCNNCCCNTTT
TTNTCNNTTNTACCNTNNNNATTNNTNNNNCCTNTTANTNNATNNTCNCNTGTCTCTNCTNNTTTTTTN
TNATTCTNTCACTTTCTCCTTCTATCTNTCATCTCNCTCAATTCACNNCTCTTTTANCANTTTTCTTTTTT
NTTNTCNATTNCTTNCCT
TTGAGNCCATANGGAGNTGCANNNCNGTGGCGGCCGCTCTAGAACTAGTGGATCCCCCGGGCTGCAG
GCTGNTGNGGNATNTGGAACANNTCGNTGTACTGAGGCNGCGGNCGCTGANGCCTCANGGAGGGCCT
GAAGGGACCCCGGCAGCGCCCCCCGGAGTGATCCAGACACTTCCAANAAGGGGCCGNCGCCACCTGT
GTATATNACCTCCTGGTACCACTGCTACACTTANCANCTTTTTTACTTTTGAGGCTTTGTNNATGTCCTG
AAGCTGCCTGNNAATTGTCCTTTNGGGGCTGACGTCACNTGTANGGGCCNTGTGAGTGGGCCNNGGGG
ACTGCAAGGCANGGCCCGCCTTNTGNTGNTNGNGGGCTCTGCTTGCTCATCCAGGCCTCTCCTGGGGG
CCACCCAGNGCCGCCCANTCCAACTGCACCCACGCGCCCGTCCCCATGAGCGACACACACGGCTCAGC
CTTTTTTTAATACNTGATCCTTTNNCGGCCACCTCGCTNTGGAGCCGNGTCTACCAGCAGNCTTATGCC
TTNAACNCGCACCTTTCNTTTNTAACGCTTTAANNTTAATTGTGATTNCCATCTTGGAAAAGTCCTTCA
ATAAAAGANNNGAAAAACCCAAAAAANAANAAAAAAAAATTTTGGNGGGGGGGGNACCCGATCCCC
CNTTTGGCCCNTNAANGGGGGATNTCTTTNTANAAANTNGTAGGNAAANAANNTTNNGGNNNNGCTN
AAAACTTTTAAATNNGGNNGGNANTTNNNTANAAAATAATACCCNCACCTNTTGNAAAANNGATTTA
AATTGGGAANAAATTGGGCAANACNCTTTTNNGNCNTTNGNCCCTTCGGAAANCNGAANTGNNACNN
AATTTTAANNNTTNTTTANCGGNNNTTTTTNNTNNGANGGAACCTNTGAANNCTTNTTNTTTATGGTTA
TNTTTNCAAGNGNTNNTAGTTTTNTNGGNTAAAAAATN
GCCTATCTGGAGCTNCACCGCNGTGGCGGCCGCTCTAGAACTAGTGGATCCCCCGGGCTGCAGGAATT
CGGCACGAGGCTTGGTGATAAGGCTCCACCAAAGCCTGTACCCAAGACTATTGACAACCAGCGAGTAT
ATGATGAAACCATAGTAGATCCTAATGATGAAGAGGTTGCTTATGATGAAGCTACAGATGAATTTGCT
TCTTACTTCAACAGACAGACTTCTCCCAAGATTCTCATCACTACATCAGATAGACCTCATGGGAGAAC
AGTACGACTCTGTGAACAGCTCTCTACAGTTATACCAAATTCGCATGTTTATTACAGAAGAGGACTGG
CTCTGAAAAAAATTATTCCACAGTGCATCTCAAGAGATTTCACAGACCTGATTGTTATTAATGAAGATC
GTAAAATACCAAATGGATTAATTCTGAGTCAGCTGCCCAACGGCCCAACTGCTCACTTTAAAATGAGC
AGTGTTCGTTTGCGTAAAGAAATTAAGAAGAGGCAAAGACCCAACAGAACACATACCAGAAATAATT
TTGAACAATTTTACAACACGACTGGGTCATTCTATTGGACGTATGTTTGCATCTCTCTTTCCCCATAATC
CTCAGTTTATTGGAAGGCAGGTTGCCACATTCCACAATCAACGGGATTATATCTTCTTCAGATTTCACA
GATACATATTCAAGAGCGAAAAGAAAGTGGGAATTCAGGAACTTGGACCACGTTTTACCTTAAAATTTA
AGATCTCTTCAGAAANGGAACCTTTGATTCTAAATATGGGAGAATATGAATGGGTCCATAAGCCCCCG
GGGAAATGGGATCCAGTAGAAGAAAAATTCATTTTATNAANGGACTGGAGAAGGATGGTATTAAACT
GGGCTGGAACCAGGCCTGGAGTTTTTGAAAACAAGANCTTNTCAAAAATGGNCGGTTACTGGATTTTT
TTAAACCGGCTTTTCTTTTCTGGACCAAAGGNGCAAGGGGCCTTGAATTTNCT
NNCNATCTGGAGCTCCACCGCGGGGGCGGCCGCTCTAGAACTAGTGGATCCCCCGGGCTGCAGGAATT
CGGCACGAGGATGGCACGTCCCCAGTGTGGGGATGTGCCGTGCTTCCTGAATTAGTGTATTGTAGGAG
TTGCTGCCCCATTCTGCTCTTCATTGTGAGAGAGGTTACACCCACTTATAATCAGTATTTTCCTGGCACT
AAAACAGCAGGGCTGGTATTTGCTGCTGCTTTCCCAGGGCAGGGTCCCTTCTTTTCGGCACTTTTGAAC
ACTTGCAAGACACAGCCCTGTTATCTAACGGGTGGCAGGCATGTGTGTCTGCACTCGCTCTTTGCCACT
ATTTTGCCCTGCTCCATGCCANAGAGCCCTGTCCCTGCCAGGCCCTGCCTTCCCAGCCCCAACTTGGGA
CCAAAGTGCAAGACGGGATCACAGGTTGGGGTGTCCAAGTGACCCCTTTTCTATAAGTGCTTCCGTGGG
CCAAGTCGACACCAGCCCCCTANCGGNGTGGGATGGNCGGNGCTTAAAGAGGAAGGGGACCAGTGTA
GCAACCTTGCCAGGGACCCCACCCCCTCCCTTACATCCGGACCCTGTGCAGTGGGCCGTGGCGATTCTC
CAAAAGGGGCCAAAAGCCTGGGCTTGCTCNGTATTCTCTGCTCCCTTGCTCTTATCNGGTGNCCATGCA
CACTTCTAAGANTGCCAAATTGCTTTTGAACTTTAAAAACTGGTCANNNTTGGNTTCCCNTTTGGACCN
ANTTTTAAAAAAANGTTCTNAATNGGGAAAAAATTNGNGGGGAAAAACNNTNGGNTAAANGGCNCCG
GGNTCCTTTGGCTTTNTCGGGGGAAAAANACANGNGCCTTTTCCGGAGGGAACCCNCTTTTTCCNCAGN
TTTTCNGNANGGTNCCTAAAACCCTGTCCACCCCCCTNCNAAANCCTTTNGCCCNGANANTNNNNCCC
GGGGTTCTCNNTAACCNTNTNTGGGCCCGNGGGTGTTTTTTTNTCGGANAATNTCTGNNNC
GCCCTATNTGGAGCTNCATNNCNGTGGCGGCCGCTCTAGAACTAGTGGATCCCCCGGGCTGCAGGAAT
TCGGCACGAGGGGCAGAAGGTTCGCGAGGAGGTGGTTACCGTGGGCAATTCTGTTGACCAGGGCCTG
AGTCAGCCCACAGATGACTCCTGCTTCGACCCCTACACGGTCTCCCATTATGCCATTGGAGAGGAGTG
GGAGCGATTGTCTGATTCTGGCTTTAAACTCTCGTGCCAGTGCTTAAGCTTTGGCAGTGGTCATTTCAG
ATGCGATTTCATCTAAATGGTGCCATGACAATGGAGTGAACTACAAGATTGGAGAGAAGTGGGATCGTC
AGGGAGAGAATGGCCAGATGATGANCTGCACGTGTCTTGGAAATGGAAAAGGAGAATTCAAGTGTGA
TCCTNATGAGGCCACGTGCTATGACNATGGGAAGAGTTACCACGTGGGAGAACAGTGGCAGAAGGAA
TATCTTGGTGCCATTGCTCCTGCACATGCTTTGGAGGCCANCGGGGCTGGCGCTGTGACAACTGCCGC
ANACCTGGGGCTGAACCCGGTTACGAAGGCTCCACTGNCCACTCCTACAACCAGTNTTTCCCAGAGAT
NCNCATCANAGGAACAAAACACTAATGTCANCTGCCCAATNTGANTGCTTCATGCCTTTGGGATGTAC
AGGGCTNACNGAGAAGAATTNCCCGAGANGTTATTTTTTTTCNCAANCCCANAGNAAACNTNGCGTTGG
NTTTGTTGCCANAGGTNCCANTCCCAAANCTGGGANTGGGGGTTNNCACTACCCCCCTTGNTNAAGTG
GGNGCCCTTTCTTTNAGCCCNTTTNNCTTNTGGNNNNAANTTTTTTTCNGTTTTTTTCCNTCAACCCCNC
CCTTTTTTCCAAGCNNTNAAANCCCTGGGAGGNGTTTTTTCAAGACTTTTTTTTTCATTANATTGGGGN
NCCAAGNTAGGTN
TTTNAGCCTATNTGGAGCTNCATNNCNGTGGCGGGCGCTCTAGAACTAGTGGATCCCCCGGGCTGGAG
GAATCGGCACGAGGCTGNATTGNCCCTANNCANGGCTGCTNCNNGGATGNCATTAAGGTNTTCTGCA
ACATGGAAACCGGTGAGACCTGTGTATACCCNANTCANCCCANCGTGGCCCAAAAAANCTGGTNTNT
NANCAANANCCCCANGGAAAANAGGCNCGTTTGGTNCGGCAANANCNTNACCGGNGNATTCCANTTC
AANTATGGNGGCCAGGGGTCCAATCNTGCCAATGTGGCCATCCAGTTGANTTTCTTGCGCCTGATGTC
CACCGAGGCCTCCCANAACATCACTTACCANTNGCAAGAACAGTGTGGCTCTANATGGACCAGCANA
CTGGNAACCTCAANAAGGCNCTNCTTCTNCAGGNCTNNAANGATATCGATATCNGNGCCGAGGGCAA
CAGCCNTTTCATNTACAGTGTCANNTANTANGNGTGCANGANTCACACCGGACNNTGGGGCAANACA
GTGATNANATACAAGCACCCNCAAGANCTCCCTTNTTGCATCATTATATGATGNGCCCCCTTTGGGA
CGTTGGCCCCCCAACACCAAAGAAATTCTGCTTCCGACGTTGGCCCTTGCCTTGCTTTCTGGAAAACTT
TCTTNCCACNACAANTGTTGTTTTCCTTCCCACNCCAANACCNTTTTTGCCTNNTNANTNTTGGNAAAC
AAGAACAAACCCCCCCTTGCTTGGCCCCCCCCCGNAACNGCCCAAAAGGNTGGGAAAACTTTTTTNTC
GTNGGNCNTTTNGAAAAATTTTTTTTTTTTNTTTTGCATTCNTTTTNTTCAAAAATTAATTTTTTTTTTTT
TTGACCAANTTGGTCCTTGAACAAAAAACCCAGGGGTTCNNTCCTTNCTTAACAAAAAAAAAAAAAA
AANCTTNGGGGGGGGGGCCCCCCCCNCCCCN
TTTNAGCCTATNTGGAGCTNCATNNCNGTGGCGGCCGCTCTAGAACTAGTGGATCCCCCGGGCTGCAG
GAATTCGGCACGAGGCTGNATTGNCCCTANNCANGGCTGCTNCNNGGATGNCATTAAGGTNTTCTGCA
ACATGGAAACCGGTGAGACCTGTGTATACCCNANTCANCCCANCGTGGCCCAAAAAANCTGGTNTNT
NANGAANANCCCCANGGAAAANAGGCNCGTTTGGTNCGGCAANANCNTNACCGGNGNATTCCANTTC
AANTATGGNGGCCAGGGGTCCAATCNTGCCAATGTGGCCATCCAGTTGANTTTCTTGCGCCTGATGTC
CACCGAGGCCTCCCANAACATCACTTACCANTNGCAAGAACAGTGTGGCTCTANATGGACCAGGANA
CTGGNAACCTCAANAAGGCNCTNCTTCTNCAGGNCTNNNAANGATATCGATATCNGNGCCGAGGGCAA
CAGCCNTTTCATNTACAGTGTCANNTANTANGNGTGCANGANTCACACCGGACNNTGGGGCAANACA
GTGATNANATACAACCACCCNCAAGANCTCCCTTNTTGCATCATTATATGATGNGCCCCCCTTTGGGA
CGTTGGCCCCCCAACACCAAAGAAATTCTGCTTCCGACGTTGGCCCTTGCCTTGCTTTCTGGAAAACTT
TCTTNCCACNACAANTGTTGTTTTCCTTCCCACNCCAANACCNTTTTTGCCTNNTNANTNTTGGNAAAC
AAGAACAAACCCCCCCTTGCTTGGCCCCCCCCCGNAACNGCCCAAAAGGNTGGGAAAACTTTTTTNTC
GTNGGNCNTTTNGAAAAATTTTTTTTTTTTNTTTTGCATTCNTTTTNTTCAAAAATTAATTTTTTTTTTTT
TTGACCAANTTGGTCCTTGAACAAAAAACCCAGGGGTTCNNTCCTTNCTTAACAAAAAAAAAAAAAA
AANCTTNGGGGGGGGGGCCCCCCCCNCCCCN
TTGANAANAGCTGGAGCTCCACCGCGGTGGCGGCCGGTCTAGAACTAGTGGATCCCCCGGGCTGCAGG
AATTCGGCACGAGGCCCNTCCCCGCGAGCGCCGCTCTGGCCGCAGTGCGCTCGCCGTGAGCTCCGGGC
TCCTGCTNAGCCAGCGCCGCTGTCNCCTCCCTCCAGTNGCCATNATGATCATNTACCGGGACCTCATTA
GCCATGACNAGATGTTCTCCGACATCTACAANATCCGGGAGGTCACGGACGGGCTGTGTCTGGAGGTG
GAGGGGAAGATGGTCAGTANGACAGAGGGTAACATCNATGACTCNNTCATTGGNGGAAATGCCTCCG
CTGAANGCCCNGANGGCGAAGGTACCGAANGCACANNNNTCNCTGGTGNCANTATTGTCNTGAACCA
TNACTTGCANGAAACCAGCTTCACAAAAGAAGCCTACAAGAAGTNCATNNAAGATTACATGAAGTCA
ATNAAAGGGAAACTTGAAGAACAGAGACCAGAAAGAGTAAAACCTTTTATGACAGGGGCTGCATAAC
AAATCAAGCACATCCTTGCTAATTTCAAAAACTATCAGTTCTTTATTGGTGAAAACNTGAATCCANATG
GCATGGTGCTCTGNTGGACTACCGGGAGGATGGTGNAACCCCATATATGATTTTCTTTAAGGATGGG
TTAGAGATGGNAAAATGGTAACAANAGTTGGCAGTTACTTTGGATCAAATCACCCGTCGGCATAACTG
AGTGGGTACTGGTTTTTGGTTCCCCCAGCACCCGGGGTTAGGCGGAATGGGACTGNTGNNAAANTNGG
GCTTTTTTNTTTTGGTTTTGAACGGTNNAATTTATTTGGGNGCCGGNGGCCTTGTTTTTNAAAAAANC
NNGCCNTGGANGGGTNGGCCAAAAAAAAAAACCCCGNTTGAAACTTATTTGGCAAAAANAAAAAAA
AAT
TTGAANAACAANCTGGAGCTCCACCGCGGTGGCGGCCGCTCTAGAACTAGTGGATCCCCCGGGCTGCA
GGNTGGGCTCAACCTGGCNTCTGGAGTTTAGTCCCCGCNCTGCTGTGNGCGNAGCTGNTGTCTGCTTNT
AGGGNCGCAGNGACTGCTGTCGCATCGGGATCTTGATCGTGATGGGTACAAAGGCGAAAGTCGGCAG
TACAGAGTTGCTCTCTTCACGTCGATGATCCTGTGCTCCCTGGCATTGGGCAGGGGTGCGGTGCAAAC
CTATGAACCGGTAGTCAGAGTNCCTGAGAATAACCCTGCCAAGCTGTCCTGCTCCTACTCGGGCTTCTC
CTCGCCACGTGTGGAGTGGAAGTTTACCCATGGNGATATCAGAGGTCTCGTTTGCTATAACAACAAAA
TCACTGCTTCCTATGAGAACCGAGTGACCTTCTCGGATACTGGCATNACCTTCCATTCTGTGACCCGGA
NAGACACGGNNATGNATACCTTGTATGGNCTCTGATGAGGGCGGCAATACCTACGGGGAGGTCACCG
TCCAGCTCATCGTGCTCGTGCCTCCATCCCAAGCCTACAATCAACGTCCCCTTCTNTGTTACCATTGGA
ACCCCGAACTGTGCTGACCTGGTTNANAAAAGAAATGGCCTTCCCCCCCCATCTGAATACAAGTGGGT
TCAAGGGATTGGAGTTAAAAAATGCTTTTGGGAACCCCAANANNCAAACCGNGGCCTTTTNAGCNA
AACTCTTTTCTTACACCCTTGAAACCAAAAAAAANANGGGGAAGTTGAATCTTTTGATCCCCGNGTCN
GGCCTTTTGACACTTGGGAANAATTCACCCTGGTCAGGCCACANNAATGGGCTTTTGCNTTNCCCCCC
NNGAAATTCNAAAACNCCCGGGGCCCCAATGGGANTGCTTTNGGGANGCTTTAATTGTTTGGGGGGGC
AATCNNGGGCCANACCTTTNTTTTTGGAAANACNTTNATTTTTNCTNGGGAACCT
TTTGANAAANTNNNNNTGGGGCNNNNNNNCNAGGGGTANNACACTTATAGGANTGGGTGNNGACANC
NACGGGCTGCTATGNAATCCTTAACNNTNNTACCTTTTCTCCTCATTNANNTCTGCCTNCCTTCCTTAN
GGGGNCTTTTNTNAANNTGACTTTTANNCCNANACCCCCACNNNNACATTTATCANACNCTTANNNTN
ACCTTTATNNNCACTCANTAGNTNTCANCACATNNNCCTCTCNNCACTCGATNCTNGACNCACCTNAC
TNTNTTCNTTTNNGATTNCGTANAANNNNTTTATTTNATNNANACATCANTTTAACCNNGCNCTCNTNCA
CGACANNCTNTTNTNCACGTNANCACATGNTNTTNNCTCTACANCCNTNNATTTNCNTCAAACTTNNA
NNAGCCNNNNANANCNNTGACGTNNNCCACNCTANTATNNCTATCTNTNNCANCCNCNGTCNNATNA
GTNTCTTTACNTCACATGANANNCNNTNGANCNTNNCATGTCTACNNANCNCTCTCTNCNTCTCTCNN
NTCATCNNNNGATATNNTNTNTCNNCTNACNCNCNCNANTCTCANACCCNGNTACTNCTTANACATGA
CTNACCTCNATCNTNANNTTCGCNCNANCCTTCNNAATNNTNTTNTCTCTNNNTCTTTNNNACTCTANC
NNTNACCGTNCTNNACCTTAANATTCNTNTTNTNTACNGNCAAGNNNACTNNCNNGTGATNANNNGN
NACTCNGGCTANATGCNNNNTANATTCTANTCCANTNNNTNCGTCTANGTCACGTANACNCTCCGAGT
NTCNACNACTATATNTGNTNNTATGANCAACTANTCNCATNNGNACTATGANATAACNCNATTCNCTAG
TACCTACNANTCAGTATCNGTAGCCNTCTTANNTNNTAANNTCTACNGATNTGGANNTCACTAGTANT
CNCGTCNNCCNNGATTNTACAAGATTTCNNCTACTCCNCTAACNATNNNACTCCAATACGTNCNCTAAG
NCNACNNTNNCTNTCCAATTAGTNNTAATCNCCCNTTNTATCTCGANNCNCCTNTNCNANCGANGNAA
NNTCCANCCCNTACNTATNTAATGANNCTNNTNTNGANTTNTATTAGTNTCATTNCTTGTACCTNTACN
TNGAGCNATTATCTNGCACTACGNCNAAGTTATNNACNNNCTNGNNACTANNAANTTNTATGCTATAN
ANCTACNCNNTATCTNCNCNCNTTCNTCTNNCTAAGTANNNTGGANNTACTANTATGCTATNTACNNN
CNNTATNTACACTCACTGGATNATNTNCTACTTCCCNTNCANTTTACACNGTANGNTCTCCCGAAAGN
NCGCCCCATCTAATTACCANNCTCCTCTCCANCGNNCGTNNGNANCACACANAACATCTACGCN
GCCTATAGGGAGNNGCATTACAGGGGNCGGCCGNTCTANAACTAGTGGATCCCCCGGGCTGCAGGAT
TCTGCCCGTAATGACTGTGGTGCTGCCAGAGGGATTTTTTTCCCCCTTTCAATGGTTTTACTTAAGTTAC
TGGATAAGCAGTTGAGTTTACATTAAAACAATGAAATGCTATATCTTGTCTATATTCTGCCTCTTCCAA
ATTGGATGTTTTTTTTTTTTGGNAAAAAATTTTCNNTTCCTTNANAANNTTTTNANGGGGGNNNNGGNA
GAAANGGGNTAAAAAAAAACCGNCNNNGTTTNAAANCCCCNGGGGNACCCNGGGGGCNNNCCNTTN
NNGGGGTGCCNNAAANCNGNNCNCAATNNANGGNNNTNACNNNACNANACCTTCCCNTNNNNNNGG
AANGGNNGGGNNGNACCAAANCNTTNCNNGTGGCAANGGNTNNNTTAACNNCNAAANTTNTCTTNAT
TAANATANGNANCCCNNGCNTANCCNNNGNCCCTAANTAAAANNGGGCCCGGGTNGNACCNTTTATN
GAAAAGNAANGGNACCCCNNCTTNCNANTTTTTTNNANNNNNTTTTTGGNNCCCCCCNNTNGNGGCCN
TNTTTTTAACNNNGGGNGNNGGGAAANCCTTTTGNAAANNGAGGAATNNCTTCTTNAAGGATTTTNNG
ATTTCCNCCCTTGNCCCNNANANTNTAGCCCANTTTTTGGGNAAAAAACTANGGGGCNNNNNGNNNG
TAAANCCAAANGGGAAAAAANGGCNCGNCTCNGGGTTTTNNAACCCCCAAAGGGGNAAACCCGGTTN
NTGCGGNGGGTNCCCTNCGGAAAAAATTNGGGGTTNNNCCCCCCCNCCCCTAANNTTTTTTTTANAA
NGTTTTTNTTTTTTNGGCTTCCAAAANNCNACCCCNTGGGGCNANTTATCNTNTAAAAAANGGNTTCCC
CCCTNAANAAAAAAAATANNTTCCCCAAAACNTGCCCCNGNGGNAAAANNTNGNCNNANCAGGAAATT
TNACC
TGAGCCTANTGGAGNNGCATNNCAGGGGCGGCCGCTCTAGAACTAGTGGATCCCCCGGGCTGCAGGA
ATTTGGGCACNAGGNTCCAGCGTGCCTGCTCCTCAGCCATGGNGCCCTCCAGGAAGTTCTTCGTTGGGG
GGAACTGGAANATGAACGGGAGGAAGAACAATCTGGGGGAACTCATCAACACTCTGAACGCGGCCAA
GGTGCCAGCCGACACCGAGGTGGTTTGCGCACCCCCCACCGTCTACATTGACTTCGCCCGGCAGAAGC
TAGATCCCAAGATTGCCGTGGCTGCGCANAACTGTTACAAAGTGGCCAATGGGGCCTTTACGGGGGAG
ATCAACCCTGGCATGATCAAAGATCTTTTGGAGCCACGTGGGTANTCCTGGGGCACTCCGAGAGAAGGC
ATGTCTTTGNGGAGTCAGATGANCTGATTGGGCAGAAAGTGGCCCATGCCCTTGGCANAGGGACTTTG
GAGTNGATTGCCTGCATTGGGGAGAAGTTACATGAGACGGAAGCTTGGCATCACGGANAAGGTCCGT
TTTCCGAGCAAACCAAGGTCATCGCANATAACNTTGAAGGATTGGNAAGGAAGGGTTGTCTTNGGCCT
ATTGAGCCCTGTGTGGGCCATNGGTCCTGGCAANGACGNGCNACCCCCGTACCAGGGCCCAGGAANG
TCACNGAAAANNCTCCCGGGGATGGNTTAAAGTCCAACNTTCTNNTNATGCNNTGGGCTNANAACCG
CCCCCATTCNTTTTATTGGGGNTTCTNGTNANCGGNNNCAACCCTGCAATAGGAATTTTGCNAAGCCA
CCCNTGGATGTGGGATNGCCTTTTCTTTNTNGGGGCGGGGNCTTTTCNTCAANACCNTNNNTTTNNTT
GACNNCNNNCAAATCCCNAAACANTAANTCCTTGTNTNTTTTTTACTACCCTTTNNTNCTNANCCCNGG
GCCTTNTNATCNTTNNNTAACNCTTTTTATTTTACTTNGCCCCCGGACTTTNCAANATGTTNNTCGGGN
NGTTTCNCTNACCCNCN
NAAAAGCTGGAGCTCCACCGCGNGGGCGGCCGGTCTAGAACTAGTGGATCCCCCGGGCTGCAGGATT
CTGCCGTAATGAACTGTGGTGCTGCCAGAGGGATTTTTTTCCCCCTTTCAATGGTTTTACTTAAGTTACT
GGATAAGCAGTTGAGTTTACATTAAAACAATGAAATGCTATATCTTGGCTATATTCTGGCTCTTCCAAA
TTGGATGNTTTTTTTTTTTNGNAAAAAATTTTNNNNTNCNTTNNNAATNTTTNNNNGGGGNCTAGGGA
NNAANGGGGTNAAAAAAAANCNGCCNNCNTTTNAANNCCCGNGGGNACCCNGGGGGNCTNCCNNNN
NTGGGGTNNCNNAAAGNCGNNCCCAACNGAAGGNNCTNANCNNANCANACCNTNCCCTNNCNNNNG
NAAGGNTGGGGNGNANCAAAACCTTTCNNNTNGCCANGGNNNGNTTAAANNCNAAANTTTTCTTTAN
TAAAANNGNANCCCNNGCNTAACCNNNGTCCCNNAATAAAAAAGGGCCCNGGTNGNNCCCTTTTANTG
AAAAGNAANGGNANCCCNNCNTTCCANTTTTTTTNACNANTTTTTGGGNCCCNCCNTTGGGGCCCCNT
TTTTNACAGGGGGGGTGGAAAACTTTTTGAAANNNGGGGTNCCACNTNAANGGATTTNGGTTTTTTC
CCCCTTGCCCCAAAANNTTNTTCCNNATTNGGGAAAAANTAAGGGGNTNTTTGNGNGCNAACCTANN
GGGGAAAAAAGGGGGCCCCCCNGGNGTTTTTTTACCCCCNNAANGGGGGAAAACCCNCCNTTNTTNG
GGGGGGTNCCCCNCNGCNNAAAATTTTGNGTTTTNTNCCCCCCCCGCCCNAAANGTTTTTTTTTAAAAA
NNNNTNTTTTTTTGGNCTCCNAAAAACTACCCCCCNNGGGGNNNTNATTTTTTN
TTNAGNNTATCTGGAGCTCCANCNCNGTGGCGGCCGCTCTAGAACTAGTGGATCCCCCGGGCTGGAGG
ANTTCGGCACGAGGCTTCAGCGTGCCTGCTCCTCAGCCATGGCGCCGTCCAGGAAGTTCTTCGTTGGG
GGGAAGTGGAANATGAACGGGAGGAANAACAATCTGGGGGAACTCATCANCACTNTGAACNCGGCCA
AGGTGCCAGCGNACNCCNAGGNGGTTNGCCCNCCCCCCACCGCNTACNTTGACTTCGCCCGGCAAAAN
CTAAATCGCAAAATNGCCGNGGNTGCCCAAAANTGTTNCAAANNGGCCANNGGGGCCTTTACGGGGA
AAATCACCCTTGGCNTGATCAAAGATCTTGGAGCCACTGTGGGTAGTCCTGGGGCACTCCGAGANAAN
GCATGTNTTTGGGGAGNGANANGANCTGATTGCGCAAAAAGTGGCCNATGCNCTGACAGAGGCACTN
GGAGNGATNGCCTGCATTGGNGAAAATTTATNTGAGNGGGANTCTGGNATCACGGAAAGGGTTTTTN
GACANACCAGGGTATCCATATNACGTGAATTATTGGAACANGGTTNTTTTGCCTATGACCCTGTGTGG
NCCATTGGTCTGGNAAGAACGGCACCCCGCAAGGGCCNAAGGAAGTNCACAAAAAGCTTCGGGGGAT
GGCTTAACCACCACCTTTTNTGATGCGGGGGATAAAAACCCGCCATTTATTTATGGGGGTTTTNGGGG
ACGGNGGCCCACCCTTGGAAGGGAGCTTGGCCCNCCCNCCCGGGGGGGGGNTGGTCTTTTNTTGGGGG
GNGGGTTGCCTTCCCTTCAAAGCCTTGNNTTTGGNTNNNNCTTCATNNNNNGGCCCAAAAAANTNNNG
CCCTGGTTTTTTTTTTAACTANCCTTTCCTGGNNNANNCCNAGGGGNNTTNGNTTNNTTTAAANAAACC
NTTNTT
TTNAGNNTATCTGGAGCTCCANCNCNGTGGCGGCCGCTCTAGAACTAGTGGATCCCCCGGGCTGCAGG
ANTTCGGGACGAGGCTTCAGCGTGCCTGCTCCTGAGCCATGGCGCCCTCCAGGAAGTTCTTCGTTGGG
GGGAACTGGAANATGAACGGGAGGAANAACAATCTGGGGGAACTCATCANCACTNTGAACNCGGCCA
AGGTGCCAGCCNAGNCCNAGGNGGTTNGCCCNCCCCCCACCGCNTACNTTGACTTCGCCCGGCAAAAN
CTAAATCCCAAAATNGCCGNGGNTGCCCAAAANTGTTNCAAANNGGCCANNGGGGCCTTTACGGGGA
AAATCACCCTTGGCNTGATCAAAGATGTTGGAGCCACTGTGGGTAGTCCTGGGGCACTCCGAGANAAN
GCATGTNTTTGGGGAGNGANANGANCTGATTGCGCAAAAAGTGGCCNATGCNCTGACAGAGGCACTN
GGAGNGATNGCCTGCATTGGNGAAAATTTATNTGAGNGGGANTCTGGNATCACGGAAAGGGTTTTTN
GACANACCAGGGTATCCATATNACGTGAATTATTGGAACANGGTTNTTTTGCCTATGACCCTGTGTGG
NCCATTGGTCTGGNAAGAACGGCACCCCGCAAGGGCCNAAGGAAGTNCACAAAAAGCTTCGGGGGAT
GGCTTAACCACCACCTTNTGATGCGGGGGATAAAAACCCCCCATTTATTTATGGGGGTTTTNGGGG
ACGGNGGCCCACCCTTGGAAGGGAGCTTGGCCCNCCCNCCCGGGGGGGGGNTGGTCTTTTTNTTGGGGG
GNGGGTTGCCTTCCCTTCAAAGCCTTGNNTTTGGNTNNNNCTTCATNNNNNGGCCCAAAAAANTNNNG
CCCTGGTTTTTTTTTTAACTANCCTTTCCTGGNNNANNCCNAGGGGNNTTGNTTNNTTTAAANAAACC
NTTNTT
TTNANAANNTCTGGAGCTCCACCGCGGTGGCGGCCGCTCTAGAACTAGTGGATCCCCCGGGGTGCAGG
AATTCGGGACGAGGGCCGAGAAGGCAACATGGGTCACCAGCAGCTCTACTGGAGCCATGCGAGAAAA
TTCGGCCAGGGTTCTCGGTCTTGCCGGGTCTGGTCCAACCGGCACGGTCTGATCCGGAAATACGGCCTC
AATATGTGCCGCCAGTGTTTCCGCCAGTATGCGAAGGACATCGGCTTCATTAAGTTGGACTAACCAAA
CTTCCTTAGATGGCTCATCCAGCACATCAACCTTAGGCGGAAACAATACTAGCTCTTTGTATATAAAAT
AAAAGTTTTCAAAACTTCAAAAAAAAAAAAAAAAAAAAAAAACNGNNGGGGGGGGGGNCCGGGCCC
CNANTTCCGNTNNNGGGGGGGNTNAAANAAANNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
CN
AACAAANCTGGAGCTCCCCGCGGTGGCGGCCGCTCTAGAACTAGTGGATCCCCCGGGCTGCAGGAATT
CGGCACGAGGGCCGAGAAGGCAACATGGGTCACCAGCAGCTCTACTGGAGCCATCCGAGAAAATTCG
GCCAGGGTTCTCGCTCTTTGCCGGGTCTGCTCCAACCGGCACGGTCTGATCCGGAAATACGGCCTCAAT
ATGTGCCGCCAGTGTTTCCGCCAGTATGCGAAGGACATCGGGTTCATTAAGTTGGAGTAACCAAACTT
CCTTAGATGGGTCATCCAGGACATCAACCTTAGGCGGAAACAATACTAGCTCTTTGTATATAAAATAA
AAGTTTTCAAAACTTCAAAAAAAAAAAAAAAAAAAAAAAAANCCNTGGGGGGGGGNCCNGGCCCCC
ANTTCCCCNTNNNGGGGGGNGGNNAAAAAANNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNC
TNNANAAAAGCTGGAGCTCCACCGCGGTGGCGGCCGCTCTAGAACTAGTGGATCCCCCGGGGTGCAG
GNGNNAAGGNTGAGNNNCCNNATNCTNANGANAAACAACCTGAANCCNAGAAGGCTGNTGGTGGCA
ANAANGCTAAAAAGGTGNACANGGTAGAAGCAATGTTAAGAAGGGGAAGCCNCACTGCAGCCGAAA
TGCTGTCCTGGTCACAGGAATTGGCANATATTCCCGATCAACCATGTACTCCAGANNGGCCTTGNACA
AGATAANGTATTCANCNNCTAAATCCNGGGTCAAAAAGAAAANGAANGTTCGNGTTCTTGCTACTGTC
ACAAANCCANTTGGAGGNGACNNNAATGGNGGTACCCCACTGGTCAAACTTCGNANAANGCCTAGGT
ATTACCGTAGTNANNATGTGCCTCGAAAACTGTTGAGTCATGGCNAAAAACCNTNCATTAAGCATGTN
AGGAATCTGCGTGCCAGCATCACTCCTGGNACCATNCTGANCNTCCTTACTGGGCGCNANATANGCAA
GAGGGTCGTNNTCCTGAACCGGCTGNGCAGTGGCTTGCTAGTNGCGACTGGGCCTCTATCCCTTAATA
NAAGTTCCTCTGCGTAGAACACACCANANNTTTGGCATTGCCACCTCCCCCAANATGGATNTCAGNGN
TGTNAAAAATGCCAGAACATTTCACTTGACCCTTACTTTAAANAAAAAAAAACCTGCGNTGNACCCCN
AANACCGGGGAGGGCGNAAATCTTGGNCCCCGAGGAGAGANATTTTTCCACNTTCCCCGNNGCCCCNC
CANGGGGGGATCNCAAAAAGCTGGGGANCCCAANAAANTTTTGCGNAAAAANCNAAAGGTGGCCCTT
NNCTTTCANGGGGTTACCTTCGGGTNCCGGTTTTGGCTTTGCNAAAAANGGANTTTNTNCCNCCCCNA
AAAAATTTGGTTTTNAAAACTTTTT
TTGANACNATCTGGAGCTCCACCGCGGTGGCGGCCGCTCTAGAACTAGTGGATCCCCCGGGCTGCAGG
AATTCNGNNCGAGGCTCCNNNTNCTTTGNTGNNNNAACCNGNNNCCNGGNNCGCNGNNGCTGNAGCN
ANTGCTAGAACAGCGGGGAAAGNGATNCTTCTCNTNAGCCAGANGGTGCTGTGNNCCTCCTATGATCT
GTGNAGAGGACTANGCGTATATNTCTANATTACACATGTGGNCTAAAGATGCCNTTACTNNNACATCC
TTTTNTGGGCTNACTCCACCNNCCACTGCGATAACCTANTTTCAGCCGNACCACACNGNCTNAAGCGN
ACCATGATNGNGACGTNACCNNTGGCACCCNTGNGNTNAAANNCAGAAAANGGCNNNGGCATTACTN
NNTTGAAGAAGGCCNCCCAANANNGCTNAACCATNACACNNAACNNTTCNATACCNNNNTNTCGNAT
CTGTATGANCCAATGANTGCTGGGNCCATGNTAGCTNTGNNGNCTGGGCGCAGGNNATGCCAGAGGA
NTGTTTTCCNGGCACCCTTACNCTCCTGNCTACTTNTTNNTNGCCCATCTNTCCCTTNAANCTNGCCCTC
TGCGTTGAACCGCATGAAAAANTTNCCAATTTGTTGCAATNCCCTTANTTTTANTTCAGNNATGTTTTT
GATTNCCCTAGCTTTCAGAACTGGACCCTTTNTTTAAAAAAAAGNAACTAAGNTNGNGATAGTTCNNT
TTGNCCCCAGGGNGGGGGAAAAAATNTTTCTTNTTTCNNGGGGTTAAAAATNTTCCCCTTGGAACCCC
TTTTTTCAAAGGGAAAANGATTTTTAANCNAANCCTTTGTTGGCAANAANAAAAAAANGGGCNAAAA
AAAAAAAANAGGAGTNCCCTNCATTTTCCGGGGTTTTAACATAAAACCCCCGTGNTTTGNCTTTTAA
AAAAAAAAAAAANT
GCCTATANGGAGNNGNATNACAGTGGCGGCCGCTCTAGAACTAGTGGATCCGCCGGGCTGCAGGGAG
AAAGAGCTCGGGGAGTCGGAGCGCTCGGAGTTTGCCACTTATCGGTCATCCTTGGTGTCCGCAGCACT
CTCTCCCTCTCTCTAGGGCGGCGACCTCCGGCGGCCGGAAAGTCACCATGTCCATCCTGAAGGTCCAC
GCCAGAGAGATCTTTGACTCTCGTGGGAATCCCACCGTTGAGGTTGATCTCTTCACCGCGAAAGGTCTC
TTCAGAGCTGCTGTGCCCAGTGGCGCTTTCAACTGGAATCTATGAGGCCCTGGAGCTCCGGGACAATGA
TAAGACGCGCTACATGGGGAAAGGTGTCTCAAAGGCTGTTGAGCACATCAATAAAACTATTGCGCCTG
CCCTGGTTTAGCAAGAAGCTGAACGTCGTGGAGCAGGAGAAGATCGACAAGCTGATGATAGAGATGGA
TGGCACAGAAAATAAGTCCAAGTTTGGTGCGAACGCCATCCTGGGCGTGTCGCTGGCTGTCTGGAAGG
CTGGTGGTGTGGAGAAGGGGGTGCCCCTCTACCGCCACATCGCCGACTTGGCTGGCAATGCTGAGGTC
ATCCTGCCAGTTCCGGCTTTCAATGTCATCAACGGTGGCTCTCATGCTGGCAACAAGCTGGCCATGCAG
GAGTTTATGATCCTTNCTGTTGGGGCCGAAAACTTCCGGGAGGCCATGCGCATCGGAGCAGAGGTTTA
CCACAACCTGAAGAATGTCATCAAGGAAGAAATATGGGAAGGATGCCCCAACCGTGGGANATGAAGG
CCGGCTTTGNCCCCAACATCCTGGAAAACAAAAAAAGCCCCTGGAGCTTGCTGAANAANGGCCTTNG
GCAANGGNTGGCTACCAGNGAACAAGGTCNTNATTNGGCATGGACCTAACTTGCCCTTGAAATTCTAC
AGGGTCGGGCAAGTATGAACCTNGGATTTAANTCCCCCATGAACCCCT
ANCNNNATCTGGAGCTCCACCGCGGTGGCGGCCGCTCTAGAACTAGTGGATCCCCCGGGCTGCAGGNT
TTTTCCNCANNAGCNCAGACTTTATTTAAGAAGTCCTTGAGATACAAAAGANGACTAGGGCTAGGTC
TACAGATNACAGAAGCTTAACCCCTGAGTGTAAGAAGCAGTNTACGGGTAAAAGGNGGTGGGAGGAG
AGGAGAGAANGGTGGNTGGCTNGNTATTTACTGGCTTTANAAAGCAGAGAGCCCAGATCCCAGCTAG
AACATCCCAGTACTGGNTATAGATTCTTTACAAAGGTGTGATACCCTCCTCATGAGCTCTTCTTGAATG
GATACTTCTTCCGAAAAATNATCATTGACNTCCAACAGGAGCAGTGGAATGTTCAGCAGACGTCAGAG
TGGAGCGGGGNTGTCTTGTGGACAAGCCAGGCTTNGNGTNGACCATGAAGCTGNTNCAGATAGGCCA
GCTCAACTTNTNTCTCCTCTNGCCGGACCNTCCGNTGTANTCTCTTTAANCAAACCAATGTTGCCTTCC
ACCGAGAGNCTTCGGGGGACCACGCCCCGCGTGTAAACCNCCTGGAAAGGGGGCACACCCTCNANTA
AAGCGCTGAACCATGAACCTTCGAAGGTGCGCACAGCAACCCTAANATAGAGGCCGGNCATGGGGCC
TNCTACCCACGAATTCACGCCNGGGGTGACTTTCCGTCGAAAACACTTTNCCGNCCCCGGAAANTTTG
GNNTAAACCCTTCCGGGGGGGGGGCCCGGNNCCCCAATTNNNCCCTTTAAGNNGGAGNNNNNNNNAA
AANNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNC
GCCCTATCTGGAGCTNCANCNCNGTGGCGGCCGCTCTAGAACTAGTGGATCCCCCGGGCTGCAGGAAT
TCGGCACGAGGAATCCCTGGGCAGGGGAACTAAGACCCCACCCCGCAAGCCGCGTGGCAGGGCCAAA
AACAACAAAAAAGAGTTGGTGGTAAGGGCGTGTGTCAGGTTGTTAGTGGGCTGCCAAGGCAGTACTC
GCTGAACCAACAAATCCCACTGCTTTTTCCTCCTGTAATCTTTTCTTTTTAAAACTTTAATCAGAGAGAA
TTTACATACCTACAATTCACCAATTTAAATTATACAATCCCGTGTAAAGTTCACAAGGNTGGTGTGATC
TTANAATACTTTTTCATCACCCCAGACAGAAACCTTGTGCCCATTANCAGACACTCTCCACATCCCCCC
AACCCCTNTAGCTTTTGGCAACTGNTAATCACTTCCTGCCTCTGTGGATCTGCTTTGGAGATTTCATAT
AAATGGAATCATAGAACATGCGACCTTTTTGTGGCTTGGCTTCTTCTTCCATTCAGCATAGTGTCTCCAA
GATAAACCTCCAAGATAAGCTCAAATCAGCACTTAATCCCTCCTTATGGCCAAGTCGTTTTCCACTGTG
TGAGTGTGCTACATCCACTCGTTTCTTCATCCATCAGTTAGCATTTGGGTTGTNTCCACTTTCTTGGGTCT
TATGAATAGGGGTNATTACCATTCAGCCACAATTTTTTGTATAGGGNGATATTTTTCAAGNTCCTCT
TGGGGTATACATACCCTAGGNAGNGGAGAATTNGCCCGGNTTNATATAAAGNCCAGNGGTTTAAACTNT
TCCANGGGANGCTGGCCCAAAACAACTTTTTTCCANNTGGGCAAGTGGCCGGTTTTNNCATTCCCCCC
ACCAANNGGNGGGGGTGGGGGGTTTTAAGTTTTTTTTCCGNNACNCCTTTANCCTNGTNCCTTTN
AAANCTGGAGCTCCCCGCGGTGGCGGCCGCTCTAGAACTAGTGGATCCCCCGGGCTGCAGGTTTTTTT
CCCAAAAGCACAGACTTTATTTAAGAAGTCCTTGAAATACAAAANAANACTAGGGCTAGGTCTACAN
ATANCAAAANCTNANCCCNTGAGNGTNAAANCNNTNTAGGGGTAAAANCAGGNGGNANCAAAGNAC
AAAANGGGGGGGGGNTGGGANTTTNNNGGNTTNAAAANGNNAAAAANCCCAAATCCNANNTTAAACC
NTCCCANCNNGGTTTAAATCTTTTACAANGGGGTTCCCNTCCTCANAGACCTCTTCTGTGTGGACCT
TCTTCCGAAAAATTATTTTNACATCCAACACAGAGCTGTGGATGTGTNCACCAGANCCTCAGAGNGGN
GNGGNGTTGTNTTGNNCACANNCCNNNNTTNGTGNANACNANNANNNTNCNNCATATGGNCNANCNA
ANTTTTTTTCNTCTTNTGCGGNCCCCTGCTGTGTCTCTTTTTACACACCCCAGTGTTCTTTTCGGAGAAA
CCTTTGGGCCACCCCCCCCCGCGTGTACACCCGTGGNNGGGGGCACACCCTAGAATNAGCNGCNGCAC
CATGAACTTAGGGGGGGCGCGCAACCCCTTAAAAAAGNGGCCGNCAGGGGTCNTTCCCNNCCAANAT
TNCCGCGANGAATTCTTTCGNNNAAGAGCTCTTNCCCCCCNTNATGCTGGNGAAANAACNCTGCGGGG
GGGCCCCCCCCCCACNNNNNCNNCTTTTANGGGGNNNNNAAAAANNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNN
AAANCTGGAGCTCCCCGCGGTGGCGGCCGCTCTAGAACTAGTGGATCCCCCGGGCTGCAGGTTTTTTT
CCCAAAAGCACAGACTTTATTTAAGAAGTCCTTTGAAATACAAAANAANACTAGGGCTAGGTCTACAN
ATANCAAAANCTNANCCCNTGAGNGTNAAANCNNTNTAGGGGTAAAANCAGGNGGNANCAAAGNAC
AAAANGGGGGGGGGNTGGGANTTTTNNNGGNTTNAAAANGNNAAAAANCCCAAATCCNANNTAAACC
NTCCCANCNNGGTTTAAATCTTTTACAANGGGGTTCCCNTCCTCANAGACCTCTTCTGTGTGTGGACCT
TCTTCCGAAAAATTATTTTNACATCCAACACAGAGCTGTGGATGTGTNCACCAGANCCTCAGAGNGGN
GNGGNGTTGTNTTGNNCACANNCCNNNNTTNGTGNANACNANNANNNTNCNNCATATGGNCNANCNA
ANTTTTTTTCNTCTTNTGCGGNCCCCTGCTGTGTCTCTTTTACACACCCCAGTGTTCTTTTCGGAGAAA
CCTTTGGGCCACCCCCCCCCGCGTGTACACCCGTGGNNGGGGGCACACCCTAGAATNAGCNGCNGCAC
CATGAACTTAGGGGGGGCGCGCAACCCCTTAAAAAAGNGGCCGNCAGGGGTCNTTCCCNNCCAANAT
TNCCGCGANGAATTCTTTCGNNNAAGAGCTCTTNCCCCCCNTNATGCTGGNGAAANAACNCTGCGGGG
GGGCCCCCCCCCCACNNNNNCNNCTTTTANGGGGNNNNNAAAAANNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNN
TNNNNAAAAGCTGGAGCTCCACCGCGGTGGCGGCCGCTCTAGAACTAGTGGATCCCCCGGGCTGCAG
GAATTCGGCACGAGGGATAACTACACCCCAGCCAATCCACTCAACACACCCCCTCACATCAAACCCGA
GTGATACTTCTTATTTGCATACGCAATCTTACGATCAATCCCCAACAAACTAGGAGGAGTACTAGCCCT
AGCCTTCTCTATCCTAATTCTTGCTCTAATCCCCCCTACTACACACCTCCAAACAACGAAGCATAATATT
CCGACCACTCAGCCAATGCCTATTCTGAGCCCTAGTAGCAGACCTACTGACACTCACATGAATTGGAG
GACAACCAGTCGAACACCCATATATCACCATCGGACAACTAGCATCTGTCCTATACTTTGTCCTCATCC
TAGTGCTAATACCAACGGCCGGCACAATCGAAAACAAATTACTAAAATGAAGACAGGTCTTTGTAGTA
CATCTAATATACTGGGCTTGTAAACCAGAGAAGGAGAACAACTAACCTTCCTAAGACTCAAGGAAGA
AACTGNAAGTCTCACCATCAACCCCCAAGGTTGAAGTTCTATTTTAACTANTTCCCTGGAACCCTATTT
AATATAGTTCCATAAATNCAAAGAGGCCTTATCAGGTATTTAAATTTATCAAAAATNCCAATAACCTC
AACACAAAAATTGCCNCCCTAACCCAAATNTTACAAACCNCCCCTTAGCTTAACATTAACCCGCCCC
NTTCCCCGGACCCCAGGAATGGANTTNCCTACGCCAGGGGGNAATGGGCCATAACCATTAATGGGAA
TNAAGGACCTTAATATGNGTTTTAGNNCCNTTAAAATTNTTTGGCCCCCTGGCCTATTGCCAGGTNC
CTTGGACCTTTTTTAGCCGGNCCCTAAATACCTTTTAAATTNTTGGGCCGGGGCNTTAGNNCCCTTNTG
GGCNAAAATNATNTTNTTTGGAAAG
ANNANCTGGAGCTCCACCGCGGTGGCGGCCGCTCTAGAACTAGTGGATCCCCCGGGCTGCAGGAATTC
GGCACGAGGCTCTTTGNTGGGCAGCCTTTGGCTTCTGTCATCGTCGTGCCTCCGCCGCTGTCGCCCCCGCA
AGGCTTTGCCGTCGCCGGAGCCACCTCCAGCGACTCGTCGCACCCGATTCTCTCCGCTTCGCTTCCCGC
CAACCGCAACCATTGACGCCATGTCGGGTTATTCGAGTGACCGAGACCGTGGCCGGGATCGAGGGTTT
GGTGCTCCTCGATTTGGAGGTAGTAGGGCAGGGCCCTTATCTGGAAAGAAGTTTGGAAATCCTGGAGA
GAAACTGGTTAAAAAGAAGTGGAATCTTGATGAGCTGCCTAAGTTTGAGAAGAATTTTTATCAAGAAC
ACCCCGATTTGGCTAGGCGTACAGCACANGAGGTAGAGACATACAGAAGAAGCAAGGAAATTACAGT
TAGAGGTCATAATTGCCCGAAGCCAGTCCTGAATTTTTATGAGGCAAACTTCCCTGCAAATGTCATGG
ACGTGATTGCAAGGCAAAACTTCACTGACCCACAGCTATTCAAAGCTCAAGGATGGCCAGTTGCTCTA
AGTGGGATTGGATATGGGTGGGGTAGCACAAACTGGATCTGGGAAAAACTTTGCTTATTTGCTGGCTG
GCATTGGNCCCATTCAATCATNAANCCNTTCCTAAAAAAAAAGGGGATGGGNCCTATTTGCTTGGNGC
TGGCNCCTAACTCGGGAACTGGGCCCAACAAGGTCCAGCNAGGGACTTGNTGAATACTTGGAAAAAC
ATTGTCNCTTTGGAAATCNACNTTGCANTTTTTGGGNGGGGCTTNCCAANGGGACCNCCAAATCCTGG
ATTTGGGAAAAAAGGNGGGGGAAATNTTTNTTTGGAACNNCCTGGGAANACTGGTTTAANTT
TNNANCACAAGCTGGAGCTCCACCGCGGTGGCGGCCGCTCTAGAACTAGTGGATCCCCCGGGCTGCAG
GGAAAAACACACCTGAAATCTTTTTGGAAACAGAAATTCATTTTAAATAGATGGCACAGAGGTAGTCT
AATGTCATGGGAAATGAGAAAAAAATATATACACATTAAAAAGCCTTCTGTCTTTTTGCCTTTTATATT
GTTAATGCATTTTGATTTAATATTCAAATAGCACTGGGTGCAGCAGTGACAAAGCAAAAATCAACTAA
AAGACTTAGATTCTATGAGATAGGCAGGCTCATCCTAATTCTCAAATACTTACTTATATGATAGTAAT
ATTCTATGCTACAACTATAAAGAAAATACTACTAGGACACCCTCCACTTTCTTGAACGTATCAGAGTTT
AGACAGAAGTAAGAGATAAACATCTATTTCTGCTTTATTGACTACGCCAAAGCCTTTGACTATGTGGA
TCACAATAAACTGTGGAAAATTCTGAAAGAGATGGCAATACCAGACCACCTGATCTGCCTCTTGAGAA
ATCTGTATGCAGGTCAGGAAGCAACAGTTAGAACTGGACATGGAACAACAGACTGGTTCCAAATAGA
AAAAGGAGTCCGTCAAGGCTGTATATTGNCACCCTGGTTATTTTAACTTATATGCNGAATACCNTCATT
GAGAAACACTGGACTGGAAGAAACNCCAAGCTGGAATCAAGAATGGCCGGGAGGAAAATATCAATTAA
CCCTCAGAATATGCNGGATGACACCCNCCCTTTTGGGCAGAAAGGGGAAGAAGGATCTTAAAAAGGC
CTNTTTGATGAAAGGTGNAAANAAGGGAAGTGGAAAAAAGTTGGGCTTTAAAGCTTCACCNGGNTTG
NAAAACCGAAAAATCATTGGNCTTNCCCTTCNCTTTNNTNGGGNAAAAAAAAANGGGGGNAAAAAGN
GGGNAAACANGGTTCNNAANTTTT
ANCANNNCTGGAGCTCCACCGCGGTGGCGGCCGCTCTAGAACTAGTGGATCCCCCGGGCTGCAGGAA
TTCGGCACGAGGGTTTTTTTTTTTTTTTTTTAAATTAAANAGTTNATTANGATTNANAAGNCTNATTTAA
AAAACNTCAGCCCNTNAANCCNTGTAAAAANAAGTNTNCAAAANGTNNGNAAAANAAANCNTNCTTN
AANCNTGTTNAAGNAAAAAANTTNTNNNAAATTNNGNATTTNNAGCCNNTTNNTTNTTNGCNTTATNA
NNCANGGGCANCCAAANAANCNTNANNTTNNGNGGGGGAAACCCNCCNAAANCCGGTTTNGTNGNA
AATCCCANCTTNNATNTNATNTTNNGCNATCGNCCNTTAAAAGNTTTCNTTTAGGGTAAANANGGNNG
NANAAAGGGCNTTTTAAAGTCCCAAATNTTNNTTNTNTTTTTTNNNANGNAANGTNTNCCNNTNNNNA
NAGTTTTTNCCNNTNGGNGGGGNTTTCCNGGCAAAGAAANCNCCAAANGGNTNTGNCNGAAAAAAAT
ANGGCCGCCCGNCNCNATGTTNTNTNGGGCGGGCGTCNTTGGCCTTTNGGGGNGGACCGNGCCCCCCG
AAANGGATCCAAGTTCNAAAAATGGGAACTTTGNAAAAAATTACCCCGGCNACTAAANTCAATCAAA
ACCCTCCCAAAAAATTCCCCNNTNTTTTAAAAAANGGNNNCCCCCNNCNAAAANNCCCCCCCGNCCTG
GANAANANAACCTNAAANCCCCCCCCCANATGTTTTGGGGAGGGGGGGCCCCTTGGCTTTTTTTTANA
AAAAAGGNGTTAAATTTTAAAAACCCCCCGGAAAAAANAAAAAAAAAACCCCCCCCTTCTNNNGGGG
GGGGGCCNCCCAACCCANNGGGGCCTTTNGGGGGGGCCTTTTNNCCNCNCCCNGGGGGGGGGAAAA
ANCANNNCTGGAGCTCCACCGCGGTGGCGGCCGCTCTAGAACTAGTGGATCCCCCGGGCTGCAGGAA
TTCGGCACGAGGGTTTTTTTTTTTTTTTTTTAAATTAAANAGTTNATTANGATTNANAAGNCTNATTTAA
AAAACNTCAGCCCCCNTNAANCCNTGTAAAAANAAGTNTNCAAAANGTNNGNAAAANAAANCNTNCTTN
AANCNTGTTNAAGNAAAAAANTTNTNNNAAATTNNGNATTTNNAGCCNNTTNNTTNTTNGCNTTATNA
NNCANGGGCANCCAAANAANCNTNANNTNNGNGGGGGAAACCCNCCNAAANCCGGTTTNGTNGNA
AATCCCANCTTNNATNTNATNTTNNGCNATCGNCCNTTAAAAGNTTTCNTTTAGGGTAAANANGGNNG
NANAAAGGGCNTTTTAAAGTCCCAAATNTTNNTTNTNTTTTTTNNNANGNAANGTNTNCCNNTNNNNA
NAGTTTTTNCCNNTNGGNGGGGNTTTCCNGGCAAAGAAANCNCCAAANGGNTNTGNCNGAAAAAAAT
ANGGCCGCCCGNCNCNATGTNTNTNGGGCGGGCGTCNTTGGCCTTTNGGGGNGGACCGNGCCCCCCG
AAANGGATCCAAGTTCNAAAAATGGGAACTTTGNAAAAAATTACCCCGGCNACTAAANTCAATCAA
ACCCTCCCAAAAAATTCCCCNNTNTTTTAAAAAANGGNNNCCCCCNNCNAAAANNCCCCCCCGNCCTG
GANAANANAACCTNAAANCCCCCCCCCANATGTTTTGGGGAGGGGGGGCCCCTTGGCTTTTTTTTANA
AAAAAGGNGTTAAATTTTAAAAACCCCCCGGAAAAAANAAAAAAAAAACCCCCCCCTTCTNNNGGGG
GGGGGCCNCCCAACCCANNGGGGCCTTTNGGGGGGGCCTTTTNNCCNCNCCCNGGGGGGGGGAAAA
NCANNANCTGGAGCTCCACCGCGGTGGCGGCCGCTCTAGAACTAGTGGATCCCCCGGGCTGCAGGGTC
TTTAAACCCTGCGTGGCAATCCCTGACGCACCGCCGTGATGCCGAGGGAAGACAGGGCGACCTGGAA
GTCCAACTACTTCCTTAAGATCATCCAACTTCTGGATGATTATCCAAAATGCTTCATTGTGGGAGCAGA
CAACGTGGGCTCCAAGCAGATGCAGCAGATCCGCATGTGCCTCCGCGGGAAGGCTGTGGTGCTGATGG
GCAAGAACACGATGATGCGCAAGGCCATCCGAGGGCATCTGGAAAACAACCCGGCTCTGGAGAAACT
GTTGCCTCACATCCGGGGGAATGTGGGCTTCGTGTTCACCAAGGAGGACCTCACTGAGATCAGGGACA
TGCTGCTGGCCAACAAGGTGCCAGCTGCCGCCCGTGCTGGTGCCATAGCGCCGTGTGAAGTCACTGTG
CCAGCACAGAACACTGGTCTGGGGCCCGAGAAGACCTCCTTCTTCCAGGCTTTAGGCATCACCACGAA
GATCTCCAGGGGCACAATTGAAATCCTGAGTGATGTGCAGCTGATTAAGACAGGAGACAAAGTAGGC
GCCAGCGAAGCCACGCTGCTGAACATGCTGAACATCTCCCCCTTCTCCTTCGGGGTGGTCATCCAGCA
GGTGTTGACAATGGCAGCATCTACAACCCCTGAAGTGCTTGACATCACAGAAGGAAACTTTTGCATT
CCCGCTTNCTGGANGGCGTCCCGCAATGTTGGCCAGCGTGTGCCTTGCANATTGGTTACCCAACCCGT
NGCATTTGTACCCCCATTCTATCATTNATGGGTACCAAAGCGCGCTNGGCTTTTGTCTGNGGANAAT
GATTACACCCTTNCCACNTTGTTGNAAAGGNCAANGGCCTTNTTNGCTGGATCCATTTTGCCTTTTGGG
GTTGGTGGCCCTGGNGNTTGTTGNCCCCNGTTGTTGCCCTTGTGGCCACCNCT
NCANNNCTGGAGCTCCACCGCGGTGGCGGCCGCTCTAGAACTAGTGGATCCCCCGGGCTGCAGGAAN
TTCGGCACGAGGGCCCAGCGAGCGCGTCTCCCAGGAGCAGTTCACAGCGTCCATGTCCCACCTGTTGA
AAGGAACTGCCGAGGAGAAGAGTCTCGTGATCCTGAACATGATTTCTGCCTCAGGAGGTCGCGTGAAA
GCAAGAGATGTCCACAAGTTTACAGAGGACTTGGTTGGCTCTGTGGTGCACGTGCTAAGCTACAGACG
GCAGCTGCGAGCTGGAGTCANAAAGCCACCCCCAGGCTCCCCATCCAGGGTGCAGGTGCTGGCTGCCC
AGCTTGTGCTCCGAAGATGAAGGCTTAAGGTGGGCGGGAAAGCTTTCTGGGACCTCAGTGGTTGGACC
GCGACTTGTGACCAGGCCCGTGCTCGAGGACTGGGGTGTTCCGGGGCTCACCACGTGCCACGTTTCTG
AGCCTGGGTCATTCCACCAGGGCTTCCTCCTCTTGCGCTCGTCCCTCAAGCTGCCACCCTGGTCCCCCG
AGCGCCAGGTGGACCCGCAGCGGGGAGTTCGCCAGCGTCTGGACGTNCTTGTCCGTCATGTACGTAAC
TCGCACCTTGCCCAAGAACCGGGGGCTNAACTTGCGCCCTACTTCTTCGCCACCGAACTTCCACGGGA
ACAAGCTTTTGCCAACTTNTGCGGGCGCCATTCGCACACGGGGGGCCCTTGCCTGGTGCTTGNTCGAA
GGACANGNNACCGGCCACGTTGTTCGGAAGGTTTCNCCTTCCTTGCTTCCTNGGAAAGTCAANCCCTT
AANTTTTTAAANGGGGAACNANNAGGGGCTTTTCTTGTTTTTCGGTTTTCCCCCGNNATNGGCGTTTTC
ACCTTNGGNGGGGNTTTNAAANAAACACTTANTTTTNCCTTANAAAANGGGGNANNNAAANTTTCCC
AAANGGTTTTGGGGATTNNGAAGAAAACCNCNGTTTCTTTTTGGNTTNGGGGNACNNNNNNTNATTTN
GGGAA
NAAAANCTGGAGCTCCACCGCGGTGGCGGCCGCTCTAGAACTAGTGGATCCCCCGGGCTGCAGGCTTA
AACGAATACATTAACGGTGCTAAACACAGACATTTTAACCCAAGTCACGACATTCTTAGCTGTAACTC
AGCTCTCACGGCCTCTTGCTCACCCATGAATGGTCCCGTTTTCCTCTTGCCGCGTGCACCTCCACCCATT
GTCTTGGTGGCACATGGGTGGAACACTTGATCTGCTCGAGTCTGCCTTCAACACACATTGCATCTTCAG
ATTTTCTACTTTTCTGNTCAAAACTAATATTCACCAAGTCAGACTTTGNGTTAATTTTATATCAGGGTAT
TGGCTGCCAGGGGGTCATCCCTAAGTGGCCTGAAGATGGACAAAGGGAAGTAACAGGCACGTGATGT
TGGCAAGGATGCTTCTAGGGCTANAGGATCAGTGGTGGGANANAGCTGCANAATCCACCANCCAGAA
CTGCAGATAACGAATCTTATGGNCAGGGGCTGTGACTGANAGAAGGAAACTGAGGNTGTGTTCTGAA
AGTACATAAACTCTCACATATACCCAGTTCTTNACCATCTCCCCTCCTNACTTTGCAGTGCCATTTCTTT
TTGCATTAGGCAATTTGCTCAGACTTTCCAGAGCCATGGCCCGATCCGTTTCTCTGGAATCCCCCACACC
CTCTGAGANGTGGATCACCACGTCCTGCAGGGNCTGNTCCCCTCCAAACTACCTTTTANGAAGATGCA
CGGACAAGGGAGGCTGTTTTCANACCCNNCAAAAGACCNNAAATTCNAGAAGCGAGGAGCCNCAACG
TGGGTAAAAACANAAAAAAANGGGCAGGGGGGNGAAAATNGGGTTTTCTTTTTGGGGGNTTTCNGGT
GTTTCTTTTTTTTCNCCNCCTTGGGATNGGCTGGTNCCCCAAAAAAATNTTTTTAAGTGNC
NGCCTACTGGAGCTNCACCGCNGTGGCGGCCGCTCTAGAACTAGTGGATCCCCCGGGCTGCAGGNGCN
ANACAAGTTGGTAAAGATAGAGTTTTTAATACCCCACTGTGTGAACAAGGAATCGTNGGATTNGGAAT
NGGAATCGCAGTCNCCGGGGCTNCNGCCNTANCAAAAATNCAGTTNGCAAATNATATTTNCCNTGNTT
TNGATCAAATGGTAAANGAACCNGCCANGNNTCGNTNCCGGNCNGGGNACCTTTTTAATNGGGNAAN
CCTAACCATCCGGCCCCCTNGGGGCTGNGTNGGCCACGGGGCTCTCTATCATTCCCAGAGTCCTGANN
CTTTCTTTGCCCACTGCCCAGGAATCAGGGNGGGTGCNCCCAAANGCCNTTTCCNGGCCAGGGNACTT
NTTTTATNNNGCATAGANGATAAAANCTNTTGTATATTTTNNGANCCTAATANNCTTNACNGGGCANC
AGNGGNGCNNGTNCNTGTAGANCCATACAANATCCCTNTTTCNCAATCTGAAGTCATCCAAGAGGGG
AGTGATGNTCTCTAGGCGCCNGGGGCACTNANGTTCATGTGNTCGGAGAGGCGGCTGNCATGGNTCAA
NAAAATCGTGGGGTGTGTTGGGANGTCNTTTGNTGTGACGACTNTACCACCGNGGGANGAGCAACAG
GCANNANGGNTGTNANNANAACNNGGACGACTGGTAATAANTNAGGAGCTTNCTTTGGACGGCGGTT
TTTCGTNTAAAAACCNCCCACCGNTGAAGNAAGAATTGTTCNTCAACCCTGGNAACCTNCTTAAAGGG
GGGGNGGGGGGGNANAAAACCNCCNNTTCCNTTTTTTNTTNGACCGNTTTCCNTCCCAAAACNGNNGG
GAATNNNTTTNCCNCCCTNAAAAAAAANGANNNNNTTTNC
NGCCTACTGGAGCTNCACCGCNGTGGCGGCCGCTCTAGAACTAGTGGATCCCCCGGGCTGCAGGNGCN
ANACAAGTTGGTAAAGATAGAGTTTTTAATACCCCACTGTGTGAACAAGGAATCGTNGGATTNGGAAT
NGGAATCGCAGTCNCCGGGGCTNCNGCCNTANCAAAAATNCAGTTNGCAAATNATATTTNCCNTGNTT
TNGATCAAATGGTAAANGAACCNGCCANGNNTCGNTNCCGGNCNGGGNACCTTTTTAATNGGGNAAN
CCTAACCATGCGGCCCCCTNGGGGGTGNGTNGGCCACGGGGCTCTCTATCATTCCCAGAGTCCTGANN
CTTCTTTGCCCACTGCCCAGGAATCAGGGNGGGTGCNCCCAAANGCCNTTTCCNGGCCAGGGNACTT
NTTTTATNNNGCATAGANGATAAAANCTNTTGTATATTTTNNGANCCTAATANNCTTNACNGGGCANC
AGNGGNGCNNGTNCNTGTAGANCCATACAANATCCCTNTTTCNCAATCTGAAGTCATCCAAGAGGGG
AGTGATGNTCTCTAGGCGCCNGGGGCACTNANGTTCATGTGNTCCGAGAGGCGGCTGNCATGGNTCAA
NAAAATCGTGGGGTGTCTTGGGANGTCNTTTGNTCTGACGACTNTACCACCGNGGGANGAGCAACAG
GCANNANGGNTGTNANNANAACNNGGACGACTGGTAATAANTNAGGAGCTTNCTTTGGACGGCGGTT
TTTCCTNTAAAAAGCNCCCACCGNTGAAGNAAGAATTGTTCNTCAACCCTGGNAACCTNCTTAAAGGG
GGGGNGGGGGGGNANAAAACCNCCNNTTCCNTTTTTTNTTNGACCGNTTTCCNTCCCAAAACNGNNGG
GAATNNNTTTNCCNCCCTNAAAAAAAANGANNNNNTTTNC
TTGGANACCNTCTGGAGCTCCACCGCGGTGGCGGCCGCTCTAGAACTAGTGGATCCCCCGGGCTGCAG
GAATTCGGCACGAGGACTTTNNGCTCCCAACNNATTCCNCTNNATNCTNGGGNTTCTATNCTATGCAA
GAGATACCTTCNTANTTGNATACTCAATCNTACNATNAATNNCTANCCCACTATGANNANTNNTAANN
CTANCNTTCTNTATTTNTTANTATNGCTNTNAAGCCNNTNAAATACNCACCTTAGGGNTTCATACGGA
AACCATNANTNNACCCTTCCACTCANCNNGGGTGCCTAATNNATGATNCCACTAAGNTTGCAACATTN
CACCCGACGCTCACATTNAANNTNNNNNGACNNACCTNNTACGTNANACCCCNTNTAATCGNCGGNTT
NGNCANAAGNAAAAANANNNGGTNTNNGTNTGTAANNGCNCTAATACNNANNTNCTTCTTNNNTNNN
GTTTTCCCNANCCTGNNTTNTCATNAANACNNAATTAGGGNGGCCANCGGCCCTTAATGGGTNCCTNT
TAATTANNTTNNTATNCGTNNNNCCCNTATGAAAAGCGAGNTTTAACANTNNCGNTNNGCANAAAAC
CTTCCTGNANNNNTAANTGCTTAATTNCNNTTTCCANCCCTNNCNNTTNTTGAAANATNNTNTCCCNAT
ATNANNTACCCTNGCCCACTTTTTTANTTTTTGTTTNNANTNCNACTCGNNNGNNGCCTTNCNTAANAT
NTNACNTTTTTTCGCGNTNNGNCCTNTNTCCNANACNTTNACTTNNGNNNCTTNGNNANCNCCNTNNT
CNTCCCACCNTCTTCNTNNTTTTCCNTTCCCGNNNGGNCATANCNCGNNAACCANNGNNTTNAANTCC
CNCGNCCCCANGGNGCTTTTATNCNTNANAATTNANNGNNNCTCGGCCCCCAANTAANTCNTTNTCNA
CCTTNNTTTTTATCCCCCNGGNTATACNNNNANNNCNTNTACNTCTTTNNCNTTNGCATTTACCNGT
TGTCATTTCTGCTGGGGCAANNCTNCCTTTANTTCNNNAANNTNCTTTNTTNCATNCC
NAAAAGCTGGAGCTCCCCGCGGTGGCGGCCGCTCTAGAACTAGTGGATCGCCCGGGCTGCAGGATTGA
NTGCNGNNTGAATNNAGCGGNNAACANGCTCGGGGCCANNNTNGCGANTAAGAACTGNNNNCGNAT
ACANGAGNGNNTTTTTTTNAAACTANNNCACNGNTTGNTGGNTNACTGGCTGNNNACAANATATCCN
ACCCTTNAACNNCTGTCCGANCTANCTGNANTTNAANGGATGGNNANNGGCAANNNCCCCTNGNGNA
NNGACTNTCNTCTAGGNGCANGGGTACAGGGGTNCTNTTCNNACNCCTCNGCCNCNTAANCTGNNNN
CANGCNANTGACCNCAGCNGGACACAGGGTNCATNTCCTGGGAAGNTCNNAACNGGTTTACATCNCAC
TTGATGNTGACNTCNAAGACTTTTCCCTAGGAAGAACCAATGGGGCACNCTTTTACNTGGAGNTGCAA
TCCNGGGATCCGCANNCANNGTTTTGACAAGGTAATTAATGCTCTGANTGAACGGGNCTNCTTCATGAT
AACCCTCCAGTTTNGGACGGCTTGCCCGACCTGTTATGNGTNTGGGCCCNANGNGCTTGCATGGNCAA
CCTTNAGGNGGCAAAAACAATCAAGTTCACCNCCTCATGGTNTAACCAAAACAATGGCTTCCTTTAAG
TGGNGGTAAAACCCCCTNNNTNNNNNNNTTTTTNCAANANAAAANGGGNNAAGGGTCCCCCNTNNGG
GGGGGGGGNAAAAAANGGNCNAAANNTTNAAACNNANCCCGGGNCTGGTTTCCTTNNAAANNNTTTN
CTTTTTTTNGGGCCANCNAACNNGNAANGGGATTNCNNNNNAANNTTNNCCCNCNNNTTGGNCCNNN
GNNGGNAAANTNNNTNANNNNGGGNNNNNTNCNNNTNCNTTAANNNCCNNNNGGNNNGGG
NAAAAGCTGGAGCTCCCCGCGGTGGCGGCCGCTCTAGAACTAGTGGATCCCCCGGGCTGCAGGATTGA
NTGCNGNNTGAATNNAGCGGNNAACANGCTCGGGGCCANNNTNGCGANTAAGAACTGNNNNCGNAT
ACANGAGNGNNTTTTTTTNAAACTANNNCACNGNTTGNTGGNTNACTGGCTGNNNACAANATATCCN
ACCCTTNAACNNCTGTCCGANCTANCTGNANTTNAANGGATGGNNANNGGCAANNNCCCCTNGNGNA
NNGACTNTCNTCTAGGNGCANGGGTACAGGGGTNCTNTTCNNACNCCTCNGCCNCNTAANCTGNNNN
CANGCNANTGACCNCAGCNGGACACAGGGTNCATNTCCTGGGAAGNTCNNAACNGGTTACATCNCAC
TTGATGNTGACNTCNAAGACTTTTCCCTAGGAAGAACCAATGGGGCACNCTTTTACNTGGAGNTGCAA
TCCNGGGATCCGCANNCANNGTTTGACAAGGTAATTAATGCTCTGANTGAACGGGNCTNCTTCATGAT
AACCCTCCAGTTTNGGACGGCTTGCCCGACCTGTTATGNGTNTGGGCCCNANGNGCTTGCATGGNCAA
CCTTNAGGNGGCAAAAACAATCAAGTTCACCNCCTCATGGTNTAACCAAAACAATGGCTTCCTTTAAG
TGGNGGTAAAACCCCCTNNNTNNNNNNNTTTTTNCAANANAAAANGGGNNAAGGGTCCCCCNTNNGG
GGGGGGGGNAAAAAANGGNCNAAANNTTNAAACNNANCCCGGGNCTGGTTTCCTTNNAAANNNTTTN
CTTTTTTTNGGGCCANCNAACNNGNAANGGGATTNCNNNNNAANNTTNNCCCNCNNNTTGGNCCNNN
GNNGGNAAANTNNNTNANNGGGNNNNNTNCNNNTNCNTTAANNNCCNNNNGGNNNGGG
TTGAACAAANCTGGAGCTCCACCGCGGTGGCGGCCGCTCTAGAACTAGTGGATCCCCCGGGCTGCAGG
AATTCNGCACGAGGAGGACTACTAATATACCGANCCCACCTANTATCCTCCCTTCTATGCTTAGAAGG
AATAATGCTATCCCTATTCGNTATAGCAGCCCTAACAATNCTNAACTCACATTNTACATTAGCTANCAT
AATACCTATTATCCTACTAGTCTNCGCAGCCTGTGAAGCAGCCCTAGGTCTATCTCTACTAGTAATAGT
ATCAAATACATATGGACTGATTATGTACAANAACCTNAACTTACTNCAATGCTAAAATACATTATTNN
AACAATTATACTTATACCCNTAACCTGGNTATCAAAAAATAATATAATTTGGGTTAACTCCACAGCAC
ACAGCCTTCTAATTAGCTTTACAAGCCTNCTCCTCATAAACCAGTTTGGCGACAACAGCCTTAATTTTT
CACTACTATTTTCTCCGANTCCCTATCCACTCCANTACTAATTTTAACCATATGGCTCCTCCCTCTAAT
ACTAATAGGCTAGCCAACATCATCTATCANAAGAAAACCTAACCCGAAAAAAAGNATTTATTACTATG
CTGATCTCACTACAACTATTGCTAATTATAACCTTTACCGGGCATNGGAACTAATCTTATTTTATATCCT
ATGNGAAGCAACACTAGTCCCAACACTCANTATTATTACCCGANGAGGAAAGCAAACCAGACGCCTA
AACGCCGGACTCTATTTCCTATTCTATACCTAAGCNGGCTCCCTAACCCTATTAAGCGCNCTAAATTAT
ATTNCNAAACCGGGGGGGANCCCTAAAATTTCCTAAAAATTAACGGGNCNGGGGGNCAANCCGGGTC
AAAAACCCTTGGAAANA
NNNNATCTGGAGCTCCACCGCNGTGGCGGCCGCTCTAGAACTAGTGGATCCCCCGGGCTGCAGGAATT
CGGCACGAGGCATCCTGGGCGGGGTCATTAGCGCCATTAGTGAGGCGGCTGCGCAGTACAACCCAGA
GCCCGTGCCCCCTCGCACACATTACTCCAACATTGAGGCCAATGAGAGTGAGGAGGTCCGGCAGTTCC
GGAGGCTTTTTGCCCAGCTGGCTGGAGATGACATGGAGGTCAGCGCCACAGAACTCATGAACATTCTC
AACAAAGTGGTGACCCGACATCCTGATCTGAAGACTGATGGCTTTGGCATTGACACATGTCGCAGCAT
GGTGGCTGTAATGGATAGTGACACGACTGGCCAAACTGGGCTTCGAAGAATTCAAGTACTTGTGGAACA
ACATCAAAAAGTGGCAGGCCGTATACAAACAGTTTGATGTTGACCGTTCAGGGACCATTGGCAGCAGT
GAACTCCCAGGGGCCTTTGAGGCAGCAGGATTCCGCCTGAATGAAGATCTCTACAACATGATCATCCG
ACGCTACTCAGATGAGGGAGGGAACATGGATTTTGACAATTTCATCAGCTGCCTGGTCAGACTGGATG
CCATGTTCCGTGCCTTCAAATCTCTCGACAAAGATGGCACTGGACAAATCCAGGTGAACATCCAGGAG
TGGCTGCAGCTGACCATGTACTNCTGAACGGGAGCCCCAAGGCCTGCCCCCTCATCACCTTGCTGTAN
GAGTCACCTTGGACTTTTCGGTTCCCCAAGGGCTGATTCTTGTCTGGAGTCACATTTTTCGTGGGTCC
TGGCTGACCCCTTTTTTGTCTTTTTANCCCTTTGGCANCCTGGATTTCTCANTTCAACAAGCCANGGCCC
CAACATGGCTTTAAACACGCCCTTGCCCCTTNAAGTTAACCCCCCCCCCAANANAACCCATTTTGGNTT
ACTTCCATTAACCCTTTTTTTTGCNCCTGGGCCAAACCCCAACATTTGGGTG
TCNTCAGNGGGNCNCGGGCGCAGCGCGCANCGTGCCCCNNACNNTCTNCATGNNATACNCAACNNTG
GNAACTGTCNTCCGNNNCNGTTNATGNNCNCNCTTAANCANCNNNANGGCNACCNCTNCGCCTCNGC
NCTNCCNNTTCNACNNNCNGCACAATATTTTNTNATNTNNTCCACCCCNCNTNCCCNTCAAANNCNGN
NNGGGGNNCCTTNNTGNCCTANNCNCNTTACCNAANCCNCTCNTNNNTANCTNCNGANNTATTANNC
NTANAANANNGCGANNCGNCCNGGNGGNGGTGTNCNTAGTANNNANNNATTGGTNNCAGNNNCCTN
GNCNNGNNCTNTNTNNNNTNNTANNTGNTGTNANNNNCGNNCGNNGNATANTNTTNNGNGNGNNGN
NGGGNAGNGNGACNGGNTNTNTNTTGTTNNNTNTGNNCNANGGNGCNGNATNAGNGCNNANNNTCN
NNNNNNNANGTNNANNNTTNTATNTNTGTNGAANANACTANNGCNCTCCAGGGNTCGNNNTNTTCNT
TNNNCNNCNNGNGNNCANNCNNGGTAAGNNNCTNAGNNGNNANNNGCNNNGNNCCCNNCCNATTNN
TGGTNNTATTNNNGAGNGNCCNCNNGAGNNCNNNNTNNGAGNNATGATCNCNCAANNNNTNGTCCAG
GCGNANAANANGNNNTANATCGNGANNGNNCNCTGNNGNNNGGANNNNATNGACNGCNNNNCTNGG
NANNGNNCCNNNCNGACNNCNANGTANCNNNNCNNANNTCNNGNGCTGNNGGGCNGCNGTCTNNNA
NNGNTANTGNGGCGNCGCNCNCANNCCACCTGTAGTCATNTCATCGNANNTNCCTCCACGNACGAG
CGNCCCCCTNGNACGGTCNNNTNCTCCTCCGCTNCCACCACNCACCNNNCCAACNNNCNATCNTAGTA
NGTCCTCCNTCCNCNANCCCTCAANGATCTTNCNNTNTTTANCCCACNGGATNCTNTGCCNGNCCGT
CGCCTNGCGNNTNCANNNGNGCCTAANNCCNNCNCNTTCNGNCGGGCGGCTCNGACGGAGTTGNNTN
NTCACTTCACCATCNNCTNCAGNACTNNCGNNGCNGTNNTATANAANANCAGCNNCANNANTAGGGT
ANNAGTTNGGANGNGCANTNNNGGTGTATATGCNANGGCNNCGGTCANCNCGNTAANANGNNGCGA
NTAGTCCAGANAGGNAGCAGATANANCGNCGCANNCNGCGTTGCGNNNNTATANNNNNTGCNANCN
ANNNCNNNNNCNNCGTGCAGCACNTGCATNCCNCGNANANANTGTNANTNTGCGAAAGCGANANAN
TANCTAACGAGANACTTNCCCNANATANAGTGNTATANAGNNNNNNTGATGNNACTNTGTCTTGNNA
NNGNGAGCCG
GGGGGGGGGAGCAGANTACNTANNATAATTTTNTNTNTNNATNNNNNNNTCAACNNTNNACACTCNA
NNCTNANTAGANCCGACNAAANACNNTCNNTAGNNGGGGGGAATANAGGACGANATATGATTNNTTT
TGGAGANACNNNTGGANNTNCATNNCNGTGGCNGGCCGCTCTAGNAACTAGTGGANCCCTTCGGNCN
TGNNGGNGNANAGAAGANNCATNGTTTTATANANTAGCTNCTTANNNCANNNANTGCNAGCGGGGGT
NNTNATTTNTTTTTNTTTNCNTATNTTANTNNCANTNNCANGTNTTCGTTNCTNGTGANTAGCAANNAC
TATNTANAACTNTNTTANTAANTNATATCTTNCNCTTGNTATTGANGTAANANTGNTNANTGANAGCA
TNNGAAGTTNNTNCNGCANCTTGGNATTAGGANGGACCNTTNTCNAATTTGNNGNAANCCNNCACGN
ATTCNTGNTTNTNTTTGNGGNGGNNTGTNNTCGNNTCANNTCTGCTNTNTATNCATTNCNCAAGANTN
CNNGAANACTTTTATTTGNNCACTGGNCNAAGNATNATAGGNGGGTTGGTAANNATANNANNTCACN
TNTTTTGATNNGCCAAANNCGANCTTTANTNNATTATTCACTNTCATNAANCATNGAATANAAANAAN
GGCNNTGATNANANTNGTTNTTTGAANCCCNTAANAACTGACTTTGATANNANTCNTTNNANNNATNG
NCCGAGAATNCAAGTGNNNNNNTTNNGTTATTAGNNAGAANNACANNNGACCNCNCNCCTTGNNNCCC
AANGCNTGANANNTAATNTNTCATATNTATGGGNGANNATAGAATGGCTCANCACNAATATACNTAT
ACNNTCNAANGACGCATNCATNTGGGCTTNACANNNNTNTAAGCNCCNAANNCGTGNACTATTATTTG
TANTTTGGGAANNNACTACANANATCCACTTTNNNACNTCTNATATTTTNNTTAATAGNGCTNTCGGN
CTNNGATTCTTGGCTGCGGANNTTATANTCTAATCCCTTTTGAGGAANGTNGNGCACTTGCACNNAT
NNATGCTACAGGTANTNNTGNAGNNATAANATATNACATGTNCATTNNACTTTGCCNNCACCNATTN
ATCNATGATANANATTATNTNTACNTGTGGGNAANTNNNTCTCNTCNCNNANTCTGNANCTATTCN
TNTANTTNCANTAGANTTTGTAATAGTANATTCTNCTATACTTCGNTCTTNNATTANTCTGTGNCANNC
CCNCCTTGANTNCCNTNANNNNTAGTGGTNGCGTCTANNNNTCGTCTNTACTNCNAGCNCCGTCNCAA
TGNANNATNNANTNTATCATNTNTTGTACGNAANNCGGTTCAATCCATTNTATGNATATTATNTAANN
NAAGGAGNACTNATATCATCNCGGTTANTATGAAACNACCNTNTGTGATGGAANGAATNANNNTCAT
GTGTTTCTANACTCCGN
GGGGGGGGAGCAGANTACNTANATAATTTTNTNTNTNNATNNNNNNCTCAACNNTNNNCACTNGATN
CTCANCNANNNCCGACNAAANNCNNTCNNTANNGNGGGGATANAGGANGACNTATGATTCNTTTTGG
AGANANCNNTGGAGNTNCANNCNGTGGCNGGCCGCTCTAGNAACTAGTGGGANCCCTTCGGNCNTGN
NGGNGNAAANAAGAGNNATNGTTTTNTATANTAGCTNCTTANNNCANNNNTGTNCCAGCGGGGNTNN
TCANTTNCTNTTNNTNNTNCNNNTNNNANTNCCACNNCCANGTNTTCGTNCNCTAGTTANTAGCNNAA
TATTTNAACTNTNTTATTAANTNATATCTTTCNCTTGNTNTTGANNTANANTGCNTNNTGANAGCATNN
GAAGTTNTNCGNNNNCTTGGNATTAGGGAGACCNTTNTAAATTNTGGAAACNTNACGATTNGNTNNN
NTNGNGGCTTGTNNTCGNNTCANNTCTGCTNTNTATNCATTCNNAGANTNCCTGAANACTTTTTTTGA
ANCCTGGTCNAAGNACTNATAGGNGGGTTGGTAACNCAAGANANCACCTNTTTNGATNNGCTCAAAAN
NCGANCTTTANTNNATTATTCACTNNCATNAANCATNGANTATAATANAATGGCTTTGTTATANTNNTT
TTTTNAANCCCCTAATAACTGACTTTGATANCANTCNTTNNANNCTNNACGAGAATNCAAGTGNNNTN
NTTNGTTNTNGNNANAATNACANNNGACCNCNCNCCTTTNNNCNNAANGCCTGNTTTNTATTNTNTCA
TNTGNTATGNGNGNNATAGAATGGCTCANCACTANTATACTNTTACNNTCNAANNACGNTNCNTNTA
GNGCTTTACANTNNTNTAANNTACCNAANNGTGTACNATTATNTNANTTNNTGGGCNNNNCACTNNNA
NACATCTCACTTTNNNNTTACTNATATTTTTNCNNATAGAACTATGTNCTNNGATTCTTNNNCNGCGGA
NATTANTANTCTAATCCCTTTTGTGNAATGTANTNCANTAACACNNNTTNNAGCTCANNGGGTANTCN
GNAGNNAATAANNTATNNTNATGTNCNANTNNACTTTGCCCTCACCTATTTACTNNATGATANANNAT
TATNCTGTATNTGNTNGNNAANTNNNNTNTCNCNCNCANTNTNCANNNATTTCNNNTAATANCNNTAG
NNNTTTGTAATAGTANATTCNNCTATACTTCTCTCNTNNATTATCCATNTGNGNAANGCCCTCCTANNA
TTNNCNTNANNCNTAGCGNTNGNNTCTNNNNNTTGATCTCNTACNNNNNGCNCCGNCNTAANGCAN
NGATNNANTNTATCATNTNTNGTACGNACANGCGGTTCNTANCCACTTGTTGNATNTTATNTAACTAA
ANGACTNACTNATATCATNNCNNNTAAATATGAAGCGACCCTNTGTNATNGNAGAATNANNNTCATG
GTGTTTTCTANANTCCGG
TTNNNAAAGCTGGAGCTCCACCGCGGTGGCGGCCGCTCTAGAACTAGTGGATCCCCCGGGCTGCAGGC
TTTGCTGAGGGTCACATTGAGCTCCCAGCGACTTCAGGGGCATCTTTAGGAGTCAGCACTATGGCAGA
AGACATCCAGGCCAAAATCAAGAACTACCAGACTGCTCCTTTTGACAGCCGCTTCCCCAACCAGAACC
AGACCAGGAACTGCTGGCAGAACTACCTGGACTTCCACCGCTGTGAGAAGGCAATGACCGCTAAAGG
AGGTGATGTCTCCGTGTGTGAATGGTACCGGCGTGTGTACAAGTCCCTCTGCCCCATATCCTGGGTGTC
AACTTGGGACGATCGCCGGGCAGAAGGCACGTTTCCTGGNAAGATCTGAACTGGCTCCATCCCACCTC
TTCTGTCCTCCGTCCTTCTCCCAAGGGTGGTGAAGGGGGACCGGGTACATGGTGATCCCCACCTTGGA
ATCCTGACTCATGGTATAACTAATAATAAATGCTCGTTGGAAACGTGAAAAAAAAAAAAAAAAAAAC
CTNGGGGGGGGGCCCGGANCCAATTTNCCCNTATNGGGGGGNNNNNNAAAANNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNN
AAAAGCTGGAGCTCCACCGCGGTGGCGGCCGCTCTAGAACTAGTGGATCCCCGGGCTGCAGGACAAA
GTGGCTTCAGAANCCCTCATGAAGNACCACATTCTAAACACCCTCCAGTGTTCTGAGGCTATCATGGG
AGGAGCAGTCTTTGAGACTCTGGAAGGAAACACCATTGAGATAGGATGTGATGGTGACAGCATAACA
GTAAATGGAATCAAAATGGTGAACAAAAAGATATTGTGACAAATAACGGTGTCATCCATTTGATTGA
TCAGGTCCTGGTTCCTGATTCTGCCAAACAAGTTATTGAGCTGGCTGGAAATCAGCAAACCACTTTTCAC
AGACCTTGTGGCCCAGTTAGGCTTGGCATCTGCTCTGAGGCCAGATGGAGAATACACTTTGCTGGCAC
CTGTGAATAACGCATTTTCTGATGATACACTGAGCATGGATCAGCGCCTTCTTAAACTTAACTCCAGA
ATCATATATTGAAAGTAAAAATTGGCCTTAATGAACTTTACAATGGACAAAACCTTGANACCATTGGA
AGGCAAACAGCTCAGAGTCTTCGNGTNCCGCACAGNTGCTGGCATTGAAAATTCATGCNTGGGNGAN
AGGAACCAGCAAGGAAAAATNGNGCCATCCATTATTCCAAGAAATCNTCAANCCACCANAAAAATCC
CNTTTTTGNAAAACTGGAACCAANANAACCNCTTCAACCANTTTTNCCTAAGTCTACTTGNAACCTGG
CAAACTTGNAAANAACCTCCTAACCCAGCCCTGGGAAATTGGGACCTTTTTTTTGGNNCCAANCCAAT
GGATGCCCTTTTANGGGGAATGGGCCTAATGGAAANAAAANGGGAAATTNTTGGATTTCGGGNNANA
AAAATNGCTTTTTNAAAAANATTTTTTCNTTTNTTAACCCGGAACCCNNGGGAGTTTTTATTTGGGAAA
AAGGGTTTTNNNT
TNNNNNAAAAGCTGGAGCTCCACCGCGGTGGCGGCCGCTCTAGAACTAGTGGATCCCCCGGGCTGCA
GGCTTTGTAACCCTCCAACAATGAATACTTCAACTTGTATCAAAACAAATAATGAGTATCCACAATTCT
AAAGGACAAACATGAACATTAATATTAATATCTCTGATCCTATTTATTGGATCAACAAACCTACTAGG
CCTATTACCCCATTCATTCACACCAACAACACAACTATCAATAAACCTAGGCATAGCCATCCCCCTGTG
AGCAGGAGCCGTAATTACAGGATTCCGCAATAAAACTAANGCATCACTTGCCCATTTCTTACCACAAG
GAACACCCACTCCACTAATCCCAATACTAGTAATTATTGAAACTATCAGCCTTTTTATTCAACCTATAG
CCCTCGCCGTGCGGTNAACAGNTAACATNACTGCAGGACACCTATTAATTCACCTAANNGGAGGAGCT
ACACTTGCNCTAATANGCNTTAGCNCTACAACAGCTCTAATTACATTCACCATTCTAATCCTACTAACA
ATTCTAGAGTTTGCAGGNGCTATAATCCAGGCCTATGTATTCACTCTCCTAGTCAGCCTATATCTGCAT
GACANCGCATAATGACACCCCCAAACTCATGCTTATCATATAGGAAAACCCAAGCCCTTTGACCTNTT
AAANGGGGCTTTGTCTGNCCTCTTAATAACATCCGGGCCTAAACCATGGGGAATTCACTTTTAACTCA
AAGGACCCTGGCTAATAAATGGGNCTAACCCCCAAATTTCCTTACCATNTTTCCCATGGGTGACGGAA
ATGTTTTCCGANAAAAGCNCCTTTCAANGGGGNCCCATAANCCCCACNTGTNCNAAAAAGGGGCNCC
CGTTTTGGGAAAAAATTNTTTTTTTTTATTCCCCCGAAAAAATNTTTTTTTTANCGGGANTTTTTTNGGG
CTTTNN
TTANAAAACTGGACTCCCCGCGGTGGCGGCCGCTCTAGAACTAGTGGATCCCCCGGGCTGCAGGCTT
TGCTGANGGNNACATTGAGCTCCCAGCGACTTCAGGGGCATCTTTAGGAGTCAGCACTATGGNAGAAG
ACATCCAGGCCAAAATCAAGAACTACCAGACTGCTCCTTTTGACAGCCGCTTCCCCAACCAGAACCAG
ACCAGGAACTGCTGGCAGAACTACCTGGACTTCCACCGCTGTGAGAAGGCAATGACCGNTAAAGGAG
GTGATGTCTCCGTGTGTGAATGGTACCGGCGTGTGTACAAGTCCCTCTGCCCCATATCCTGGGTGTCAA
CTTGGGAGGATCGCCGGGCAGAAGGCACGTTTCCTGGGAAGATCTGAACTGGCTCCATCCCACCTCTT
CTGTCCTCCGTCCTTTCTCCCCAGGGTGGTGAAGGGGGACCGGGGTACATGGTGATCCCCACCCTGGGA
TCCTGACTCATGGTATAACTAATAATAANTGCTCGTTGNAAACGTGAAAAAAAAANAANAAAAATCT
ACNGGGGGGGGCCAGNCACCATANTCCCNTAAANGGAATCNGGAATNCCCNNTNGNNCTTNNNTNTN
AGTNNGNGGNTGNTNCTNACCCCACCCNTTTTTTTTTNTTTTNNNNGAAATCTCANNCNCCTTNTNTT
TNTTTNNAANNANCNNNNGGGGGGCNNTTTTTNNTTAAAAAANNNGGNNTTNCCCNANGNNNANNNG
NNTTTTTNAAANTNNNNAACCCNTTNNTTTNAANTTGGGGGNTNANCNNNNNGGGNCCNNNNNNCNA
ATTNNNNNCNCCNNAANNNCNGGNNANNNNNNNNNCCNNNAAAANNNCNCCNNGNGGGGGGGGGCCC
CNNNNNNCCCNANNNTNNNNNNNAAAAANGGGGNCCCNNNNTTAAAANNAANNNNNNNNTTTTTT
NNNNNNNTTNNNNNAAA
CCCNACTGGAGCTCCCCGCNGTGGCGGCCGCTCTAGAACTAGTGGATCCCCCGGGCTGCAGGGATATA
TCCCCTCGTCAAATTCTGTAAACTAAAAGTCAATGTCGATTGAAGGAAAGGGAGTCAGAATTCATTCA
GGGAAAATTTAAGTGATTACTTCAAACTCTTACTATTATTAAAACTTTTTTATAAATAAATGCTTCTATT
TGATTAACTATATATTCAGGGTAGAATTATAAATGAATAATTCTGGCACAAAATAGGACTTCATAAAAT
ATTAGAAAAATAAATGAAATCATTGAGATGAAGATAAAATATAAGTCTTTATGATTACAATCTCTGTA
AACATAAGTGAATGTGAGGATTCATGTCTTAAATTCATCTCAACTGTGTAGAAAGCCACTTCCTTACCA
TGTTCCCATCTCTGTCCCTAAAGAATATCTGTGATTTTTTATCCAAGACTACTGTGCTCTCTCATTTGGT
TGAATGCCAAGCATCCACAGGCTGACTGTTGCTTCTGGACATCACTGATGCTACTTAGCAGCAGCAGC
AGCAGAACACACCATAGCAGGTTCTGTTTCTTTTTTTTTTTTCCTTTCNAATTTTATNCCANCATTATGG
AAATTCNTTCCCTCTACCCNCTAAATTGGGGACAAAAACCTCNGTTTTTCCCATATNGCCNGANCTTTT
CATTTTTATTCCCTTTTTGGANGCATGNGATTAATTNTATNCCTATCTTTAAAATCCNCCTTACATGCNG
GCCTTTGGGGNNCCCCTTTNGAAGGGAAGGGAATANNGGGAATTAATTTTGGTNNTAAAAGAAAAGT
NNATAAAANAATAAAGANGGNCAAAATACCCCCCCCTGGTTTTTTGGANGGGNGGCTNCCTTTTTTTA
NAATCCTAACCGGGCNTTGGCTNCCNAAAGGCGNTTNCCTANAAAATTTTTTCCCTTTNTGGTAAGGN
CCTTTTTCNNAAAAAATCCCCTCAAANAT
TNNNACAAANCTGGAGCTCCACCGCGGTGGCGGCCGCTCTAGAACTAGTGGATCCCCCGGGCTGCAGC
NNTAGTNGAAGCTGGGGCANGAACAGGCTGAACCGTGTNCCCTCCCTTAGCAGGCANCCTACCCCATG
CAGGAGCTNCAGNANATCTAACCATTTTCTCTTTNCACTTANCAGGAGTTTCCTCAATTTTAGGAGCCA
TNANNTTCATTACAACANTTATCAACATANAGCCCCCNGCAATGTCACAATACCAAACCCCTNTGTNC
GTATGATCCGNAATANNTACCGCCGTACTACTACTACTCTNGCTCCCTGTATTANCAGCCGGCATCACA
NTGCTATTAACAGACCGGAACCTAAATACAACCTTCTTCGACCCGGCAGGAGGAGGAGACCCTATTCT
ATATCAACACTTATCTGATTCTTTGGACACCCCGAAGTCTATATTTTAATCTTACNTGGGGTTTGGAA
TAATCTCTCATATCGTGACCTACTACTCAGGAAAAAAAGAACCATTCGGATATATGGGAATAGTTNGG
GCTATAATGTCAATCGGATTTCTAGGTTTATCGNATGAGCCCACCATATATTCACTGTCCGGAATAGAC
GTCGACACACGAACCCTACTTACATCAGGCCACTATAATTATTGCTATTNCCAACCGGGGTAAANGTC
TTCAGCTGAATGGCCACACTTCATGGANGNAATAATCAAATGGGGCTCCTGGTATAATGNGAAGCCCT
AAGCTTTTATTTTCTTAATTTACCGGGANGGGGGTTTAACCTGGGAATTGNNTTAACCCACCTNTTTCC
CTNNNANATTGGTNTTTACGAAACCATTACTTACGTTGGNNGNACAATTTCCCCTTTTGTTTTAATCAA
AAAGGAGCTGGTTTTTGCNTNTTTATANGGGGGGGATTTGTTATTGGATTCCCCCNTTTTTNTAAGGGT
TTAACCTTCAANGNTTCCTGGAACCCAAAATCCCCTTTNNNAANTTTT
GATCTCAGAGGGGGNGGNTGCGCNCNNCNNTACNNAACANACNGCGTNNTACTTNNNACGCNNNCTA
TCNCNCANNTATNNCACNCGTANTACGTGCNCCNGCGTCACTCCGNACTGCNNGGNACCNCCGCTNNT
CCNATCAATTGATANNNTCCCTNCNNNTTTTNNCTCTCTTCTTCNTNTCCTACTNNACTNNTNANTGNN
ANNATGNANCNNGNCAGTGCTANCTANGCATCANCNNTTNGCAATCACCNNNAATCANATANGGTGT
CTGCTNCTTNCANTCTNCNNNGTNGCCNNTCGCCGNTCCNCGACGCCNNGCNNCCCAGNANCNTNCTA
TTCTAANNANNATANAGNNGGNGACNNAGACCGGGNCCGGNTNCNTNTNNNNNTATNNAAGATAAN
NNNATNGGCNNNNGNNNGNNTGTANNANTNAATACTNTNNCNANGNNNNNNNNGATNGNGNANGAA
NTTNNNCNNTGTGGGNNNGNNCACNTNGTANNNAANTGTNTNNNTNCNNATACTGGNNGGNCCNGAA
NTNNANTNANTNCNANNNNCNNGNGNNGGNNNNTTNNCNTNNCTCANNNNGNGNTNNNGTGNGGNC
GNGGNGANNTCNNGNNTANANGNGNGNGTTTNNCNTCNGANNNNNNTNGANNTCNNNTNNNCGNTG
CGNANNGNAACNNTCNNATANNNNTGTNCTTCTNANNCTNACCNTTGANGNNGNNCGNGGCAANTNN
AAAATCATNNANNGNNNANANGNCNGGANNGNNNTNANNNGANCCNGNNGAGATNNGGNACCAGC
NTNAAANGNNTNANNAGTNNTAGANGNANAGGNNNTCNTGAGANNGCTAANGGNAANCCNNGNTNA
GTNNGCANNANNCNNCGAGANCNTTNTATGTTTTTANNNGACANCANAACCGNTNNNCNTCGNCATC
TNTTNCCCTNNNAATCNATNGGGCCGCCATGTTNACCANNANANCCANNTCCCNGTTNTCNNNNCTNC
NTNNTTTAACTANNNNAATCNACTANGGGNTCTGCAAGCNNACNCANANCNNGTTNCNCNNGTCNCN
NACTGNAAGNCNTNAANCNCANTNNANAGTNGCNGNANNNTCNCGCGGNNGCNNNTNCANAGNNNT
CGNCTNAGCAGCANAANCNGTNGTACNTCACAGGANAACNCCAACGGGNANCGTGGAACNNNNNNN
ACNANNNANTNACNANGTTCAGATCANNAGCGTANCNNNNNNGNCGNNTAGACNNANAGAGTNCNC
TGTNAATATACGNACNCNGNNCNGANANTANAANANATGCTGNGNCCTGTGACNTNATNCACAGACN
GANTACNCNCAAACNNAATNATATCGCNNNCNNNAGANAGCATNAGNNANANNACGTNCAGTNAGA
GAGANNANAGTGATAGNGCGCNANNGACCG
TTTNANCCTATCTGGAGCTNCACCNCNGTGGCGGCCGCTCTAGAACTAGTGGATCCCCCGGCCTGCAG
GGGAAGTGGGAGCGTTGTCTGATTCTGGCTTTAAACTCTCGTGCCAGTGCTTAGGCTTTGGCAGGGGTC
NTTTNAAANNCAATTNNTTTAANNGGGNCCTTGNCATNGNANNGANNTNCANAATNGNAAAAANNGG
GANTNGNNNGGNAAANANNGGCCAANNNNNNACNNGNCCTNNNTTNGAAANNGNAAANGGAAANT
NNAGNGGNNNNCNTATNNAGGCCNCTNCNTNTANCAANGGAAGAANTNNCCANTNGGAAAACCATG
GGCGAAAGNATATATTTGGGCCCTTTGCTTCTGNGCATTCNTTTGGAGGCCACGCGGCTGTGCGCTGTG
ACACACTGTGCCNCACACTGTGGCTCTNNACCNGGAAACAAAGGGCTACTGTGCCCTCTTANACAANC
TGTTTTCCCAGATACCCNCTCAGAANACACACTCTTGTGTCCTGNCCCCNNNAGAGCGCTATGCCCTTT
GATGTNCACGCTNNCACAGAAAATACCNNGAGAGTTATTTCCCCCNCAGAAAAAACCGTGGATCTCTG
TGACGGGCATCCTACNTGGTGGNGTGGTGTTCAGACCNCCCCATNATNGGGTGCTTNTTTAATACCCTT
TTTTTCTTGAGGGAACNTCTTCAGCTTCTTTCNTCATCNTCNNAACTTTNTTCAAACAATCATCCCTGG
GNNGGGGTTTTTANAAATNTTTTTTTTTCATTNGGNNNGCAAGCANGGGTNNTTTTTTTNTTTCTAAAA
AAATTCCTTGCCCCCNCAANTNATTTTTTNTTGNAANGGGAGTCTTACCTTGCGNGNTTNGGNTTGGGG
GGGCCATCCGGGAGGGAAAATTCCTCCACCCAANAANAAAAAAAGGGNTTTTGNANGGGAATTTTTT
CCCCACCNTTTTTTTTNN
TTTNANCCTATCTGGAGCTNCACCNCNGTGGCGGCCGCTCTAGAACTAGTGGATCCCCCGGCCTGCAG
GGGAAGTGGGAGCGTTGTCTGATTCTGGCTTTAAACTCTCGTGCCAGTGCTTAGGCTTTGGCAGGGGTC
NTTTNAAANNCAATTNNTTTAANNGGGNCCTTGNCATNGNANNGANNTNCANAATNGNAAAANNGG
GANTNGNNNGGNAAANANNGGCCAANNNNNNACNNGNCCTNNNTTNGAAANNGNAAANGGAAANT
NNAGNGGNNNNCNTATNNAGGCCNCTNCNTNTANCAANGGAAGAATNNCCANTNGGAAAACCATG
GGCGAAAGNATATATTTGGGCCCTTTGCTTCTGNGCATTCNTTTGGAGGCCACGCGGCTGTGCGCTGTG
ACACACTGTGCCNCACACTGTGGCTCTNNACCNGGAAACAAAGGGCTACTGTGCCCTCTTANACAANC
TGTTTTCCCAGATACCCNCTCAGAANACACACTCTTGTGTCCTGNCCCCNNNAGAGCGCTATGCCCTTT
GATGTNCACGCTNNCACAGAAAATACCNGAGAGTTATTTCCCCCNCAGAAAAAACCGTGGATCTCTG
TGACGGGCATCCTACNTGGTGGNGTGGTGTTCAGACCNCCCCATNATNGGGTGCTTNTTTAATACCCTT
TTTTTGTTGAGGGAACNTCTTCAGCTTCTTTCNTCATCNTCNNAACTTTNTTCCAAACAATCATCCCTGG
GNNGGGGTTTTTANAAATNTTTTTTTTTCATTNGGNNNGCAAGCANGGGTNNTTTTTTTNTTTCTAAAA
AAATTCCTTGCCCCCNCAANTNATTTTTTNTTGNAANGGGAGTCTTACCTTGCGNGNTTGGNTTGGGG
GGGCCATCCGGGAGGGAAAATTCCTCCACCCAANAANAAAAAAAGGGNTTTTGNANGGGAATTTTTT
CCCCACCNTTTTTTTTNN
TTGANAANANCTGGAGCTCCACCGCGGTGGCGGCCGCTCTAGAACTAGTGGATCCCCCGGGCTGCAGG
CTNTGCTGANGGTNACATTNAACTCCCNNCGANTTCAGNNNCATNTNTNGGNGTGANCAATATGGNNC
ANNACATNCNGGCCAANATCAGGANCTACCANACTGNTCCTTTNGACNGNCGATTCCCCAACCNNAA
CCANACCANGAACTGCTGGCAGACTACCTGGACTTCCACCGNTGTGACAATGATNTGACCCNTNNTG
NCNGNAAANTTTCTGTNTGTGAAGGNANCNGCGNGTNTTNTNNTCCCTGTGGCCNNTAAAAANNCTG
NNNNNTTNGGANACNANCCNAACANAAGGCACGTTTCCTGGGANGATCTGAACTGGCTCCATCCCAC
CTNTTNTGTCCTCCNTCCTTCTCCCCAGGGTGGTGAAGGGGGACCGGGGTACATGGTGATCCCNACCCT
GGGATCCTGACNGATGGNATAACTAATACTAAATGCTCGANGGAAACGTGAAAAAAAAAAAAAAAA
AATTTTTTTTNGGNNAANNAANGNNCGNNTGGGGNCCNNNNGGNGGGGGGTGTNTNCAAGGGTNGGN
NNTTNNAAAAANNTTTTTTTTTTTTTTNTTTTTNAAAAAAANNNGGNNNNGGTNANTTNGGTTNNTTTT
TNCNCANCCCCCCCNCCNNTTNGAAAAAAAAANNCCCCNGGGGTATNTTNGGGGGTTTTAANGCCNTT
TTNGCCNNNNNCCCCNACCCCCCCNNNTGNGGGGGGGGGAATTTNNTTTTTTNNCCCCCNNTNAAAAN
GNNNNNCNNNGGCCCCCAAAAAAGNGNGGGGGGGGGNNCCCCCTNCTTNNNGNACANAAANNAACC
NTTNANNCCNGGGGNNTTNNNNTNANAAAAANCGGGGGNTTTTTTNTGGAAAANTNNGGNGGGGGGN
AAANNAAAANTNGGGAAAAANAANAATTNTTTTGCCT
NAAAGCTGGAGCTCCACCGCGGTGGCGGCCGCTCTAGAACTAGTGGATCCCCCGGGCTGCAGGCGCTG
CCCGGCCGGCCGCCCTGGCGAACGTGGCCGACACGAAACTGTACGACATCCTGGGCGTCCCGCCTGGA
GCCAGCGAGAACGAGCTGAAGAAGGCATACAGAAAGTTAGCCAAAGAATACCATCCTGATAAGAATC
CAAATGCTGGTGACAAATTCAAAGAAATAAGCTTTGCATATGAAGTACTATCAAATCCTGAGAAGCGC
GAACTATATGACAGATATGGAGAACAAGGTCTTCGGNAAGGTAGCGGTGGAGGTGGTGGCATGGATG
ATATTTTCTCTCACATCTTTGGTGGAGGATTATTTAGCTTTATGGGTAATCAGAGCAGAAGTCGAACCG
GCAGAAGAAGAAGGAGAAGACTTGATGCCNTCCCCTCAAAGTTTCTTTANAAGACCTGGTNTATGGGC
AAGACANCCAACCTACAGCTTAACCAGGAATGTNCTCTGGTATGGCTTCCANGGGCCAAGGGGGGNA
GGCTNGGACCTGTCAAAAAGTGTAATNGCCTTGGTCGGGGGCCNAGGGGGNCNCATTAAGAACAAA
ACAGCTGGCTTCCAGGGATGGNGCCACCAAATCCAGCTGNGGGGGTCTGGACTGGAATNGGANAAGG
GAAAGGTAATTTATGGAAAAGGGNCCGTNGTAAAAAANGGGANGGGAAAAAAAGGGGNTTAAAGAA
AGTCCAANAATTNTTGAAGTCCCCCTNAAACAAAGGCCTGNAACCTTGGGCCGGANAATTCCNTTTA
CTNGGGGAAACCAAACCNGGNCCCCCNGGGGNNGGNANCCCGGGANACATTGTTNTTTTTTGCTTCCA
NAAAAAAANAACCTTGGNGGGGTTTCCCANAANACCGGGAATGGATTTGGCCCATTGACAATTTAA
AAAAAAGGGACTTTGGTGGAAACCNTTTTT
TTNAANACAANCTGGAGCTCCACCGCGGTGGCGGCCGCTCTAGAACTAGTGGATCCCCCGGGCTGCAG
GAATTCGGCACGAGGGTTTGCCGCCGGGACACAGGNGNCGTGAAAACCANNGTTAAACCTAAGCCAA
AATGGGAAAGGAGAAGACCCACATCAACATCGTTGTCATTGGGCACGTAGATTCAGGGAAGTCTACC
ACGACTGGCCATCTGATCTATAAATGTGGCGGGATCGACAAGAGAACAATTGAAAAGTTCGAGAAGG
AGGCTGCCGAGATGGGAAAGGGCTCCTTCAAATATGCCTGGGTCTTGGACAAACTTAAAGCTGAACGT
GAGCGTGGTATCACCATTGATATCTCCCTGTGGAAATTTGAGACCAGCAAGTACTATGTTACCATCATT
GATGCCCCAGGACACANAGACTTCATCAAAAACATGATTACAGGCACATCCCAGGCTGACTGTGCTGT
CCTGATCGTTGCTGCTGGTGTTGGTGAATTNGAAGCCGGTATTCTCCAAGAACGGGCAGACCCGTGAG
CATGCCCTTTTGGCTTACACCCTTGGGTGTGAAACAACTAATTGGGTGGGCGTTAACAAAATGGATTCC
ACTGAGCCGCCCTATAGTGAGTCCGNTANAAANNNNTTTTGGNNNNNCCTTGTTGCCNCCNGTTTTNN
TTANAANGTNCNAANGGGCCCGGGNNGGNNNNCTTTTNNCCCCAAAGNTTTNNGCCAAAACTGGTTT
NAAACCCTNNTTTNATNCCCCNNNGTCTTTTTTTNCNNNNNNCCNGGTGCAANNTNATGGNNCCTCCT
TNNNNGGGGCCCCCNCCNTNCNNNTNNAACCCCNNTGGTTTTNNNNCCCNNATNNGNAACNTCNNTCT
TCAANNTTGCNCTAANAAACTACCCGTNTNNNNNNNNTNNTCNTNANNGNNNTNNTTNTNNNGNGGG
GNNTNTNNCNNNNATTTTNCCCTTGNNGGC
TTGANAANACTGGAGCTCCCCGCGGTGGCGGCCGCTCTAGAACTAGTGGATCCCCCGGGCTGCAGGCC
CGTGTGGCCCTNTAGTGAGTNGNATTACATGCCGAGCTCCAGACAGACATCAACGAGCTGACCAGCGA
CCTGGACCGCTCAGGAATCCCCTACCTGGACTATAGGACCTATGCTATGCGAGTCCTGTTCCCGGGCAT
CGAGGACCATCCTGTCCTGCGTGAGCTGGAGGTGCAGGGCAACGGGCAGCAGCACGTGGAGAAGGCG
CTGAAGCTCTTCGCGCAGCTCATCAACAACAAGGTGTTCCTGCTGACCTTCATCCGCACCCTGGAGCTG
CAGCGCAGCTTCTCCATGCGGGACCGCGGCAACGTGGCCTCGCTCATCATGACGGGCCTGCAGGGCCG
CCTGGAGTACGCCACCGACGTCCTCAAGCAGCTGCTGTCTGACCTCATCGACAAGCACCTGGAGAACA
AGAACCACCCCAAGCTGCTGCTCCGGAGGACAGAGTCTGTGGCAGAGAAGATGCTGACCAATTGGTTT
GCCTTCTCCTGCACAAGTTCCTGAANGANTGTGCCGGGGAGCCGCTTGTTCATGCTGTACTGCGCCATC
AAGCAAGCAAATGGAGAAGGGCCCATTGACGCCNTACGGGCGAAGCACCCTATTCGCTGACGAGGAA
CAACTTATTCCGGGANCAAAATNGAACTACCAANAACCTTGATTTTGAACTGNGTTAAATCTTGACAA
NCAANAACAGCCCCGGAAATNCCAAGNGAANGGNGTTNAAACTTGGGAACCCCNTCACCCCAGGTTC
AAAGGANAAAAAATCCTTTGATGCAAGNNTAAAAAAAAACGTGGCCCCTTAATTTCCAAAGGGGGCC
CAAGGGGNTTTGGGGANATTGGGACTTTNGGAANNGGGGGNAAAAANGG
GCCTATANNGAGNCGNATNACAGTGGCGGCCGCTCTAGAACTAGTGGATCCCCCGGGCTGCAGGAAT
TCGGCACGAGGTTTCTTTTCTCTTTTTCAGTAAAATGCAGTGATTATTTTTATACTTTATGTTACTTAAC
TGTATATTATAGATATTCTTTCTCATAACTGCTTGGGCATCTAACTAGTACTTCTTTACAGAGGAAAAA
AAAATTTCCTAAGGGCAGATCTACCATATTTCATTTACTCATTCCTATGTTCACAGGAACTCACTTTGT
GTGTGTGTATGTATTGGAGGACTGCTTTTGTTTTGATCTTGTTGCTTTTGGTTTTTGTTTTGGCTGCCA
AAAGTGATGGCACAAATATCTAGGAAGACTTTTTTCCCAAGATACCATATCCAAAAGAAACATAAAGC
TTGACTGTCATTTTTCAAAGCAGATAAGAGGGTTTTAAAGGATGAGTTTCCTGTGTGAGGACAAGATTC
TGCCGTTTGACTGTTGTTGTTAAAGTCGGTAAGTCATGTCTGACTGTCTGTGACCCTGTGGACTGTTGTT
TGCCAGGCTCCTGTGTCCATGGCATTTCCCAGACAAGAATACTGGAGTGGGTTGCCATTTCCTTCTCAA
GCAATTTGATATGGTTCTTATTTTTATTCCAAAAGCTGAACGTGCTTTGCCGGGCCTGGTTAGTGAACA
TAGGACTGGAAACGCCCTCCTGGTTTTAAGAAATGCCTCTNGNCCAATCCAGTTGCCGCTAGCCACGN
TTTCCTCTTTAATCTAAAGGTATNTAACNATTAGAATAAAATTCCCGTTCANNTTCCTTTNGNNCCTTT
NCCCCATATCAATAGGCTCCNTGGTGGTNNNCGTGGTTTGAACTTTTAATTGCNTGGGCCCCTNGAAAT
GTNTTTTNANNNCAATTTTTTATTATNACCCNGATGGGGGNNCNGGGGCTANCCCCCTTANANCCCCC
GGTTCTTGAAANNTTTTCCCCTACTGGCACGGCNTTTTGAACNCT
NNCAANNCTGGAGCTCCACCGCGGTGGCGGCCGCTCTAGAACTAGTGGATCCCCCGGGCTGCAGGAA
TTCGGCACGAGGGGCCTGTACGCATGCGGGGAGGCTGCCTGCGCCTCGGTGCATGGTGCCAACCGTCT
GGGTGCAAACTCGCTCCTGGACCTGGTTGTCTTCGGCCGGGCGTGCGCCCTGAGCATCGCAGAGTCCT
GCAGACCCGGAGATAAAGTTCCCTCGATTAAACCAAATGCTGGGGAAGAATCTGTCATGAATCTTGAC
AAATTGAGATTTGCCAATGGAAGCATAAGAACATCGGAACTGCNACTCAACATGCAGAAGTCGATGC
AGAGCCATGCCGCGGTGTTTCCGTGTGGNGAGTGTGCTGCAGGAAGGCTGTGANAAGATCAGCAGCCT
CTACGGAACCTGCGGCATCTGAAGACGTTCGACAGGGGGAATGGTCTGGAACACTGACCTGGTGGAG
ACCCTGGAGCTTGCAAAACCTGATGCTTTTGTGCTCTGCANACCATCTACGGAGCCGGAGCCCCGGAA
GGAGTCGCGCGGCGCCACCCCAGGGAGGACTTCAAGGAGAGGGTTGACGAATACNATTACTCCAANC
CATNCAGGGGCAACAAAAAAACCCNTTTGACACACTTGGAGGAACCCACGCTNTCTACGTGACATCA
AAANTGGGAAGGGCACCCTGAGTCAAACCTGTATCGAAAAACTTTTAACGAAAACTGATTGGNCCAT
GTTTCCCCAANCCTTCGNTTTTTAATGAGGANAAANGNGGTTTCCCTGGCCCNGGNAATAANNANAAA
ANCCCNCCCCCCCGGTTTAGGGNNNNNCCCCCNTTTTTTNATTGGGCCCTTNNNNAANNNGGGGGGGG
CCTTTTNNNAAAAAATNCTNNCNNNAGGCCCNNNNNTTNNCCNNNNGGGGGTANNNNNNNNNTTNNC
CCCGGGNNTTTTTTTTNNAAAAAAAAAA
TTCTCAAGGGNGGTGCGGANGCGAGCCTCACACGTCCCNNTNNACNCACCCNCTGGANNTCTCCNTNA
CNTGCGGCNTGAACGCGANCCTCNTATNCNGTGTGCNCCCCCNAACNNGANATANCAANNCCAGCCA
CNNAGANNTTTCGCTATTCNTANANGGTNCANTTANGNAACCCNTGGGGNNTTCCTNCATNGCTCAG
NACNTTCTAAGTNNNNTNAAGCGCCNNANNTCNATGCNCCAGCTTNGCCGNAACGNNAGNGNGGANCA
AGANNGNNNCCNNANNNAACNGNGTTTGNNGCTNCNANGANNNAAANGNNAATNTNTNNTNNNGGG
NANTGGGNNCCNGGNGGGNNTNNTNNTNNATNGNGNGTNNAAGNGGNCAGTNCAANTCTCANGNNN
NNCNATTGNNGGNNGNNNACGGCNANTNGCNTGNGNGNGTNGNAGNAGCGNNNNTGNTTCNCGNGN
CGNCTAATGCCCCTATGGGNNNNNNACNNTNNNNANNTNNANGGGGNCGGCCNGGCNGNNNTNTNTG
AGATGNNNTGNGTTGGATNNGNNNNNACATNNTTNNTANNNNGGTNNNANANNGNGNNNNCNCNNG
NNGTNNTNTATNNNNNGGNANNNTNGGGNGACNNNGAGTNATNNNGTNNNNTNNNGCAGAGGNGAC
CGNNGNCAGNNTNNGTNTNTNNTNAANCCGGNCCCGAANNGCTAATGNGTGGNTCNANNANANTCAG
NNNNNNNNGGNNGNATNNNNNAGGTNNGANNNNGNNGGNNGGGNNCANNATNCGGGGGGANTCCN
TTNNNCTNTGGNGNTTAGNGGANNCCANNGNACGTGNTNGNNNNGACANNNNACCCNNCNCNTTTGT
AAGCTGTCNANAAAGNTAGGGCGGCCAACCCNTCTCCCTCTNTCCTCTTCATNNAAGTCTTACCNCN
ANGGCCNNNACCGNGCCTNNTGCACTTNNTGCNGNNNGCCTACGNGNCNGNTGCGGTACNGTGNNNT
NNNTGCNNGGAANNCNNNCNCGTNCGTCCGNNANTACNNNNGCGGCNCTNCNNAANTCNNCNGTACN
CNCTGCNGCNGCACGNNNGNNGTGGTNCTGNACANAGTTNGCTNTGAGNAGTCNCNGNNCNNTNTNN
ACTNANTGNAGCGTAGCGGAGAGANCAGAANGGCANGTCGCCGCTGNNCNGTAGNNANNCTGNCAN
NGCCGGNTGNNTNTNTTGTCAACGNAGCGNNCNCCACAGTNGNGANTGCNCTNGNGNGCGGCGNNGT
NAGGNNAGNNAGTCGTNTGCNCNNNGNGANATCGCGAANCNTNANANAGCCG
TTNAGCCTATNTGGAGNTNNANNNCNGTGGCGGCCGCTCTAGAACTAGTGGATCCCCCGGGCTGNAG
GAANTTCGGCACGAGGGCCACACCGAAGCCTACCCCAGCTTCCTGTCTGTGGAACACTCGGGCCTGGG
GCTGGNCCCTGCCTATGGGTNTTTACAAANCCCNTACGGAATNACNTTCNANCCGCNGCNGCCANNAC
CNAGGGCCNGCNAGGNNCCAAANCCGGGGNANCNNACCCGNNCNTTNCCCNNANNNGNNCNNGNTT
CNNCNTTNAACNNGGCTTCCTNGAACGAAANAANNTTCCGGTTTAAAACNNGACTTTTAGAAAGGGA
NNCCCACCCNGNAACNGGCTTGGAANTTTTACCNAACTTNACCAANTTTCCACTTTTNAACGGTTACG
CGAACACATTCACANGGCACANGGAAANNCCCATCCACNGGGAGAGTGTCCGGGTTATATCAAAACA
GGGGGGACATNNAGAGAGTGNGAGCACCTTTTANNCTATANCACTGNGNTCTCTTTGCCCCAAANNCC
NAGAGAGNGTGTGGCTTTGCNCGGGAAAAGANTGTGGANACCCANAAACCCCCGGGCGGGGACTTTT
NACANAGAGGTGTGAGTGNNCCCCCGANTANTGCACTGGNGGATAAAAAATATATATNTCTTCTANTN
TTACACCCNNCNTTTTGGACTACNCNCGAGGNCCCCGCNCCCTTCTCTTNTTTNNANGNGGTGGGGAG
GGGNGGGGCCACAGGACTTCCTCCCCCGCTTGTGGAAGNCCCCAAACCCCGTTGNCCCCNGGGGCCGC
NGNTTNTTTTNTTTTTTTTTTTNAAGACCCTCTTTTTCTTGNNGGGCCCCTTTTTTTTNCCCCCCCCNGT
TTTNTCCTTTTTNCNTNCNTTTNNNNNAAANNGGGGGAAGGGGGAACCTTTTTTGGGAAAAAAAATTC
NNTTNTTTTGGAAAAA
TTNAGCCTATNTGGAGNTNNANNNCNGTGGCGGCCGCTCTAGAACTAGTGGATCCCCCGGGCTGNAG
GAANTTCGGCACGAGGGCCACACCGAAGCCTACCCCAGCTTCCTGTCTGTGGAACACTCGGGCCTGGG
GCTGGNCCCTGCCTATGGGTNTTTACAAANCCCNTACGGAATNACNTTCNANCCGCNGCNGCCANNAC
CNAGGGCCNGCNAGGNNCCAAANCCGGGGNANCNNACCCGNNCNTTNCCCNNANNNGGNNCNNGNTT
CNNCNTTNAACNNGGCTTCCTNGAACCAAANAANNTTCCGGTTTAAAACNNGACTTTTAGAAAGGGA
NNCCCACCCNGNAACNGGCTTGGAANTTTTACCNAACTTNACCAANTTTCCACTTTTNAACGGTTACG
CGAACACATTCACANGGCACANGGAAANNCCCATCCACNGGGAGAGTGTCCGGGTTATATCAAAACA
GGGGGGACATNNAGAGAGTGNGAGCACCTTTTANNCTATANCACTGNGNTCTCTTTGCCCCAAANNCC
NAGAGAGNGTGTGGCTTTGCNCGGGAAAAGANTGTGGANACCCANAAACCCCCGGGCGGGGACTTTT
NACANAGAGGTGTGAGTGNNCCCCCGANTANTGCACTGGNGGATAAAAAATATATATNTCTTCTANTN
TTACACCCNNCNTTTGGACTACNCNCGAGGNCCCCGCNCCCTTCTCTTNTTTNNANGNGGTGGGGAG
GGGNGGGGCCACAGGACTTCCTCCCCCGCTTGTGGAAGNCCCCAAACCCCGTTGNCCCCNGGGGCCGC
NGNTTNTTTTNTTTTTTTTTTTNAAGACCCTCTTTTTCTTGNNGGGCCCCTTTTTTTTNCCCCCCCCCNGT
TTTNTCCTTTTTNCNTNCNTTNNNNNAAANNGGGGGAAGGGGGAACCTTTTTTGGGAAAAAAAATTC
NNTTNTTTTGGAAAAA
TTTNAAAAANTCTGGAGCTCCACCGCGGTGGCGGCCGCTCTAGAACTAGTGGATCCCCCGGGCTGCAGG
CGGACATGCTGGTCGTGCGAGTGGTTGAGATGTCCATCCTGGAGCCTGACGTGCCCCTCCCACTAGTC
CTCCTCTCCCAGCCCAGCTACCTCAAAGTCTTCGTCCCCAAGCACACCCAGGAGCTGCGGCTGGAGCT
GCAGGGCTGTGTGTCCAATGGGAGGCTGGGCTGCCCTGTGCACCTTACCGTGGGGTCGGCCACCCTGC
CAGGAAACTTCCAGAAGGTGCTCACTTGTAGCGATCCCACCACGGCCTGCCGCCTGTTGTTGCCTTNAC
CACCNTGGNAACGGNGGTTGCAAGTGACGGCCAAAAGCCTGGCGGGGCCCCGCGTGTTGGNNACGTT
AATNGCCGGGGTTGCCTTNACAGCTTGCANACCGTGAATTGCNAACTTCAANGGCNTTTTGAAAAANA
AGCCCNAACCAAANNAGCACCGCNTCCACGGGCCCGCCGCCCTTGAGCCCCAACACCCAGGACCTGG
ACCAAAGTGGCNAGGCAGGCAGNGGCCCTTTNTGNTTAAGGAGCTTCCCCATCCTGCGGGAAAACAT
GGATGTGGNGTTTTGGGCCCTTTCCGGCCCCTGGAAAGGGCCTTGGNGGCTCGTGCAAGTCGGACATT
GCCTTAAGNGATGAAAATTGCNCCTTTAACACGGGCATGGGACAGNGGGGGGCTTCCTTGAACANTTT
TTTGGGAACCGTAAAACCAAAACCGNGGATTGGCCAACAACCACCCTNGGGGGGGGGCCTTGTGNGA
ACCGNCCGGGTTTGCCCTTTTNTTNAATTTTAAAAANTTTGNTTTAAATTGNACCAAAAANCTTTTTTT
CAAGGGGTAANCCCCTTTGGCCCCTTGANNGCCTTNGTTTTCCCAAAAGNCAAACCTTAAAANTTCCC
TTACCCAAANAAAAANAAAAATTGG
NAAAAGCTGGAGCTCCCCGCGGTGGCGGCCGCTCTAGAACTAGTGGATCCCCCGGGCTGGAGGCCGN
CAGGACACAGGTGTCNTGAAAACCACCGTTAAACCTAAGCCAAAATGGGAAAGGAGAAGACCCACAT
CAACATCGTTGTCATTGGGCACGTAGATTCAGGGAAGTCTACCACGACTGGCCATCTGATCTACAAAT
GTGGCGGGATCGACAAGAGAACATGGTTCGAAAAGTTCGAGAAGGAGGCTGCCGAGATGGGAAAGGGCT
CCTTCAAATATGCCTGGGTCTTGGACAAACTTAAAGCTGAACGTGAGCGTGGTATCACCATTGATATCT
CCCTGTGGAAATTTGAGACCAGCAAGTACTATGTTACCATCATTGATGCCCCAGGACACAGAGACTTC
ATCAAAAACATGATTACAGGCACATCCCAGGCTGACTGTGCTGTCCTGATCGTTGCTGCTGGTGTTGGT
GAATTTGAAGCCGGTATCTCCAAGAACGGGCAGACCCGTGAGCATGCCCTTTTGCTTACACCCTGGGT
GTGAAACAACTAATTGTTGGCGTTAACAAAATGGATTCCCTTGANCCNCCCTATAGCCAGAANANATN
CNAANAAATTGTTAAGGAAGTCAGCNCCTATNTTAANAAAATTGGNTACAACCCCGACACAAGTACA
TTTGGGCCAATTTNTGGCTGGAATGGGGACACCATGCTTAAACCCAAGGCTAATTTGCCCTGGTTCAA
GGGATGGAAAGTCNCCCGTAAGGGCCGGCTTGCCNATGGGAACCCCCCTTGNTTGAAACCTTTGGA
ATGGANTTTTGGCACCAAATTTGNCCAAATGGAAAAAANCCTTGGGGTTGGCCTNTTNCCGGAANGTN
NTTAAAAATNGGGGGGGTTTGGGNCCTGGCCCCTTGGGGGGCCCGGNGGGNAAACTTG
TTNANAAAATCTGGAGCTCCACCGCGGTGGCGGCCGCTCTAGAACTAGTGGATCCCCCGGGCTGCAGG
CGAACAGTCCTCACGCCGATCTCTTTNCTGGNTCCCACCCGCGCCAGGTGACACGGAGAGCTGACACC
ATGGTTCATCAGGNGCTCTACCGGGCGCTGGTCTCCACCAAGNGGCTGGCGGAGTCCGTNCGGGCTGG
TAAGGTGGGCCCTGGCCTTCGGGTGCTGGATGCGTCCTGGTACTCGCCGGGCACCCGCGAGGCCCGCA
AGGAATACCTGGAGCGCCATGTGCCCGGCGCCTCCTTTTTTGACATANAGGAGTGTCGGGACAAGGCC
TCGCCTTACGAGGTGATGCTGCCCAGCGAGGCGGGCTTCGCCGACTACGTGGGCAGCCTGGGCATCAN
CAACGACACGCATGTGGTGGTGTACGATGGTGACGACCTGGGCAGNTTCTATGCGCCGCGGNTCTGNT
GGATGTTCCGTGTGTTNGGCCACCGCACCGTGTCCGTGCTCAATGGTGGCTTCCGGAACTGGCTGAAG
GANGGCCACCCGGTGACATCTGAGCCCTTACGCCAGAAGCCAGCCATCTTCAAAAGCCACGCTGAACC
GCTCCCTGCTCAAGACCTACGAGCANGTGCTGGAAAACCTTCGAATCAAAAAAGGTTTCAACTTGGTG
GAATTCACNGGCCCCANGGGCGGTACCTGGGCACACAAGCCGGAANCNANATCAGTAGGACTGGAAC
TCGGGCCACATTCCGAAGGCTTCGGTCNACATTGCCCTTTCATTGAACTTTCTTAAANGGAGGAATGG
NTTTTTNAAAAAAAACCCAAAAGGAAACCTCCCTTGCCCTTGTTCCAAGGGCCCAAAAAAGGNGGGA
CCTTTAANAAAAGCCCCTTTTTTTGGCCCCATTGGCCCGGGAANGGNGTCACCCCNCCTTGCCCCAATT
GGCCCTGGGTTGNTTTAACCTTTNGG
AGCCTATCTGGAGCTCCACCGCGGTGGCGGCCGCTCTAGAACTAGTGGATCCCCCGGGCTGCAGGAAT
TCGGCACGAGGGTTTGCCGCNGNGACACAGGTGNCGNGAAAACCACCGTTAAACCTANTCCAAANTG
GGAAANGANAANACCCNCATNAACATNGTTGTCATTGGNCACGTAGATNCAGGGAAGTCTACCACGA
CTGGCCATCTGATCTATAATGTGGCTGGGATCGACAAGAGAACAATTGAAAAGTTCGAGAAGGAGG
CTGCCGAGATGGGAAAGGGCTCCTTCAAATATGCCTGGNTCTTGNACAAACTTAAAGCTGAACGTGAG
CGTGGTATCACCATTGATATCTCCCTGTGGAAATTTGAGACCANCANGTACTATGNTACCATCATTGAT
GCCCCAGGACACATANACTTCATCAAAAACATGATTACAGGCACATCCCAGGCTGACTGTGCTGCCCT
GATCNTTGCTGCTGGTGTTGGNGAATTTNAAGCCGCTATCTCCANGAACGGGCAGACCCGTGAGCATG
CCCTTTTGCTTACACNCTGGGTGGGAAACAACTAATTGGTGGCGNTAACAAAATGGATTCCACTGGAC
CCCCCTATAGTGAATCNNNATTACCANNNGGNGGNGAACCCTNNAANCAAANAGGNTTTNANNTGGG
GGGNNTNNCGGNCCCNNGGGNNNGNNNNNNNGNNNNNNNNNNNNNNNNNNNNNNNNNNNNNNNNNNN
NNNNNGNNNNNNNNCNNNNNNNNNNNNNNNNNNNNNNNNNNNNNNNTTTNNTNNNNTTNNNNNNNN
NNNNNNNNNNNNTTNNNAAANNNNNCCNNNNNNNNNNNNNNNNCCNNTNNNNNNNNNNNNNNNNA
ANNNNNNNNNNNNNNNNANNNNNCNNNNNNNNNNNNNNNNNNNNNNNNGGGNNNNNNNCC
GNNCCTTANTAGNGGANGNGCGGCAATNTAACNGNGGGGAGGTCCCCANTTACCATTAATCTGCCTN
NCATCCCCCNCGGAGCCTTGCATGAGCANATCNCCGGTCATCNGAGGAGAGNTTTCACGNGGTCTCTT
AAGAATACANGTGNCNGTGTCCTCAGGGGGGATCTGNTAATNTANTTTCTCCTTCTNTNAANCCGTNA
NAANNNNCNATACACNAACGTANANTNAAAANTAANNNTCTTNNANATTTCTTNCAGNAGCGNNCAN
CCGCGCNNGCCNACTCNGTNTCAGNCNNNCTNTTNNANNANGTAAAGNAGATNNTNGTGGAAANGGN
AGGNTANNCNANNNGNGNATTGTNCAGCAGCTNNTNNGACTNGNGNANGNCGNNTTTATNNAGGATC
TNTAAAGNNNANNNNNNNNNTNNNANTTGCTNCTNNTATNTNGAGGNGGNANNNNNGNGNANTGNN
TGANGATNAATTNNNNGCGNNANNNNANTTTANGNGTCGGNGTNACNNCNCNGNAGNCGCATNANN
ATTANNTGGNGTNTNTATNTTTACNGNTATNCTATNTGTANCCTCNNTNTNNNCGNCNNCNGCNCN
NGNTTTNNTNTTNTNCTATNATNNGNTCACNCNNNGNGNTCNNAGNNNNGGCTGGAGCTACGATANC
GAATCGATCNNGTNNNGCNCGNNTNTGNTNCANTGCNNGGNNGCGCTGGCGNCGCTAGGCNTAANNN
TTNCNNCCATNAANNGANGCAGNCTACNNANACNCACTGACNGNTGATTACATTAGATTANGANNAN
NGANCANCNGNANTTGTCANANACTGCGTNCNNCAACGNAGANGNNNNGCTNGANACNNTGNGNNT
CTNTGNNCTNGTCAATNGNACANTNNGNCNGGNAGGCAGTAGNNNAGTAATTCNCGCGNCANTNTA
GCTNNGGACNCNGGANGGTNANGCCCNNNNNTGTNNAAGNNGNCGGCNATNNGNCNTANNNTNAN
CANTGNNGCGCGCACNTTGTNATCTCCNCGCAGGNAGAATANCGAGNCGNNNGTGNGNGCTNTCNGC
TATGGCNGNANTCGCNNNCGTAANAGAGTGATNCNCGNNGGNNTANGCCGNGTNNNNNANNCGCGN
NTGTGNNNCGNNNCGNGNCGCNGACAGANTNCCNCGCNCGCGANTNTNCANCTANNNGNANCANAG
AGNGCTNGTNTNTNNNNGAAGTTACGNNTTANNCAANCGNTAGTNNGTNTNANCANCGTCCNCNCGA
ANTNCCACNTCGTTGGTGTNTTANTNGNANNCGTCGNGTAGNTATCANTGNTGANAGCGNGNGGNNG
CTGGCGTNNTCGGATANTNTCNGNGNCANTNAGNATGCGCGNTAGACAGTNANCNGNGGNNGNCATN
AATCNGNGNTNGNCGNACNGTNNGCGACANAGTNANNTCGTGNCGANCAGTGACTNTCACGNTCNCN
NNANTATCACNTNCCG
GANTNNANTGNNGCCGANTCNCNGGGCGGCCGCTCTAGAACTAGTGGATCCCCCGGGCTGCAGGANT
TCNNCACGAGGANATNTCTTTAANAGTAGGAGAGGGGCCAATAGCTGNCATCATGACTCCAACTCTT
AAACCNGGNTTNACNGATTANTAANNANGNCNNTCATNANTCACNANANCCTGGNTCNTTATACNCN
CGCTCNCATTNCNAACNNTTTNTNCNCNTCNNCNNNNTNTCTTNNNTACANTNCNTNNNTNCATCTNN
NANCACNTCTAGNNNTANTNCCGACATTCNCNCNNCNTNTNCGNNANNCTNNNTNCCCGCTNTCTNCT
CCNNAACTANTCTGNCATATCNCNGAGCCNCGTANTATGNACTTNNTNNCTNNNNAAATANCNNTNTT
NCCTTCTNANCCNNGACCTCTTNCTNCNTNATNNNCANCCNNNACCNNTCTTGTTTNNNNNGCCCNAT
NNTNTTNNNGNTNANNCTNNTCANNTNNGCNTTACTCGAATNCGTATNNATGTTNACNTTCTTTNCTT
TNTNTCCNTTATNNCTATNNCCANNCNGNAACNCNTTTTNCNTNGCNCTCTATNCACNGTCNTCCNCC
CCGTCTGTNCNACNCTNCTNTCNCGGCTNNCNTANTNCGAGNCNNCCNTTANCTGGGTCNCGNGCTTC
NNCNAGCTNGANGNNCNTCNNCCCCNANANCTCCNTCCCCTTCTCTCTGTATCCCTCNTGNNTNCANN
TCTATNNNTCNTATTNNTNAGTNCNANCANNCACCCNANTANTNNNATNCCNTNNTACCNCNNCTCAN
NNCACCNNATNNTNCATNTACTAGNTTCNTTNCTCGCACGNNNCTCGCCTCTCTGTCCTCNTTNTNCTC
TGTATTNCNNTNNCCNTGTTTANNCTTCCTACATCGTTNNTTTNCNTNTCTCTTTNTTTCTCNTTTTNNA
CNACCTACTNCNTTCTTCGTCTTNCTTATCTNNTACTCNNTTTCNGTTCCCCTNCTCATCCCTCTCNAAG
NNTCNCNTAACNTTCTCTAGCTTTTNNTNTGNATTCTCNGNNTCCCTCTCTCCTACNTACCCCNNCNGN
AANTCTCTNNACCATTCTTCNNCCTCGGCTCC
GCCCTATCTGGAGCTNCANCNCGAGNGGCGGCCGCTCTAGAACTAGTGGATCCCCCGGGCTGCAGGA
ATTCGGCACGAGGAGAGGTGCAACTTTCTTCGGTCGTCCCGAATCCGGGTTCATCCGACACCAGCCGC
CTCCACCATGCCGCCTAAGTTCGACCCCAACGAGATAAAATGTACCTGAGGTGCACCGGGTGGGGGAA
AGTCGGGGNCCCGNTCTGGCCTTGNCCCCAANAANCGGGCCTTTGGGNCTTGCTNCNAAAAAGGGCG
GGGATGACNTNGCCNANGNNACTGGNGANTGGAAGGGNGTGNAGANTACNGGGNAAGTGNCCNTTC
NNAACNAANNANCCCNAANTGNNGGGGNACCNTTGNTTNTGNCCTNNNNATTAAANCCCTTAAGGN
ACCNCCCAGGGACCGAAAGAAGCCGAAAAACCTTTAGCNCNNNGGAAACNTTACTTTTGATGAAAAN
GNCAACNTTGNCCNGNANANGCNGGATTGGNCTCTAACTNNANAACTTTNTGGAACCNTTNAAGAGA
TANCTGGGGACCGCGCCACATCTGTGGGGCTGCAATGTGTGATGGGGCGCCACCCCTCATGGACATCA
TTGANGATATNNAACACTGGCGCCCATGGAAATGCCCCCCNTTTGTTAAANAACTGCGCAGGGNNAAT
ATAAGGNGCCCTTTTTCACACCNCAAAAAAAAAAAAAAAAANNTTNGGGGGGGGGGCCGGGNAGCCCA
ANNTTTCCCTTTNNNNNGGGGGGNGNNNTTAACNCNNNNNNNNNNNCNNTCTCNCTNNTGTNNNNNN
CNTNTNNNTNNACNNNNNATNNTNCTCTNNNCNTTNTNTNNTTCTNNNNANATTNNNNTTNNNTNNNT
TTNTTNTNNNCTTNNNCNNTTGNNANTCNNNNGNTTNAANNNCNTTNNCATTNNNNCNTNNTNNNTN
GCTNNTNCNNNTNNTTNCTTNTNTNTNNNTC
TTNAGCCTATANNGAGNCGNATTACAGTGGCGGCCGCTCTAGAACTAGTGGATCCCCCGGGCTGNAGG
AAAAAAAATTGTAGGAAGTCAGCACCTNTNTTAAGAAAATTGGCTACANCCCNGNCACANTATGCATT
TGTGGCNAATTCTGGGCTGNANGGGGNNCAACNTNCTAAACCNAATGNCTATNANNCCNNGGTTNAG
GGANNGNAAGGCANCCGNNAGGGNCGGNATNGCCNNGGAACCNNCCTNGNTNNAACTTTNGATTGG
ATTNTNGCANCNACTTGCCCNANTGNNNAANCCTTGNNNTTGGCTTTTCCNGNNGNCTATTAAAATGG
GGGGATTGGTACTGGCCCTGTGTGGTCATGTGTGAGACATGGTGTTCTCAAANACTGTGNATGGTGGT
CANCTNTTGGTCTCACNGCNATGTGNACACACTGAGNATGANACCTTGTGTAAAATGCNCCNCTGTAA
NCATTTTGTGAAAACCTNTNCNGNGGACACATGTGNGCTTTTTATNTGNAAAAAACACGTTTTGTNTA
AAAATATCCCCCTTGGNANATNTGNGGTGTTGGANCANCACAAATGANNCCCCCCCTNGGANACNTN
NCTNNGTTTNCANNCTCNCGANGNTTTTTNTTGNAANCTNNACGGGCNNNANCAGAGCTGGTATNTTG
CCCCNNGTGNCTGNGANTNGTTGNCACCACANCTCANTAGCTTNCANANTNNGNCTGNAGCCCNAGA
NAAGAAAAAATAGATNNTTCCNTTTTTTTGGAAAAAAAANTNGCAAGGAAAGNNGCNCTCCATATTCT
TCNAAAANAATGGTGGACGGACANCCCNCNATTGGTTTATTGNTTCGCTTGGGGGAANNCCCTNTTTT
TTTTGAAAAAGNANNNTTTTGATTAANTCCNCCCCCNGGGGGNCNCCNTTTTTTTNNTCCCCGACNNA
GAAAAAAAANCNACCCCNTTGTTGGGGGGNNTTCCNAANNAGCGGNCCTNAAAAGAGANGNTTTGNA
TNCTTGCTTTNGGAACNCN
TTNAGCCTATANNGAGNCGNATTACAGTGGCGGCCGCTCTAGAACTAGTGGATCCCCCGGGCTGNAGG
AAAAAAAATTGTAGGAAGTCAGCACCTNTNTTAAGAAAATTGGCTACANCCCNGNCACANTATGCATT
TGTGGCNAATTCTGGGCTGNANGGGGNNCAACNTNCTAAACCNAATGNCTATNANNCCNNGGTTNAG
GGANNGNAAGGCANCCGNNAGGGNCGGNATNGCCNNGGAACCNNCCTNGNTNNAACTTTNGATTGG
ATTNTNGCANCNACTTGCCCNANTGNNNAANCCTTGNNNTTGGCTTTTCCNGNNGNCTATTAAAATGG
GGGGATTGGTACTGGCCCTGTGTGGTCATGTGTGAGACATGGTGTTCTCAAANACTGTGNATGGTGGT
CANCTNTTGCTCTCACNGCNATGTGNACACACTGAGNATGANACCTTGTGTAAAATGCNCCNCTGTAA
NCATTTTGTGAAAACCTNTNCNGNGGACACATGTGNGCTTTTTATNTGNAAAAAACACGTTTTGTNTA
AAAATATCCCCCTTGGNANATNTGNGGTGTTGGANCANCACAAATGANNCCCCCCCTNGGANACNTN
NCTNNGTTTNCANNCTCNCGANGNTTTTTNTTGNAANCTNNACGGGCNNNANCAGAGCTGGTATNTTG
CCCCNNGTGNCTGNGANTNGTTGNCACCACANCTCANTAGCTTNCANANTNNGNCTGNAGCCCNAGA
NAAGAAAAAATAGATNNTTCCNTTTTTTTGGAAAAAAAANTNGCAAGGAAAGNNGCNCTGCATATTCT
TCNAAAANAATGGTGGACGGACANCCCNCNATTGGTTTATTGNTTCGCTTGGGGGAANNCCCTNTTTT
TTTTGAAAAAGNANNNTTTTGATTAANTCCNCCCCCNGGGGGNCNCCNTTTTTTTNNTCCCCGACNNA
GAAAAAAAANCNACCCCNTTGTTGGGGGGNNTTCCNAANNAGCGGNCCTNAAAAGAGANGNTTTGNA
TNCTTGCTTNGGAACNCN
NAAAAGCTGGACTCCACCGCGGTGGCGGCCGCTCTAGAACTAGTGGATCCCCCGGGCTGCAGGAATTC
GGCACGAGGCCGGGTTTCGGATCGCGAGTGCGCGCCGCAGCGGGCACCGCGGTCCGGAGCTATGGCGG
TCGTCGTCCGAGTAGTTTATTGTGGCGCTTGAGGCTACAAGCCCAAGTATGTTCAGCTCAANAAGAAG
TTAGAAGATGAGTTCCCTAGCCGTCTGGACATCTGCGGCGAGGGGACTCCCCAGGTCACCGGCTTCTT
TGAAGTGTTCGTAGCGGNAAAGCTGGTTCACTCCAAGAAGGGAGGCGATGGCTACGTGGACACGGAG
AGCAAGTTTCTGAAGCTGGTGGCCGCCATCAAAGCCGNTTTGGCTCAGGCCTGATGTGGCCTAAAGGC
AGAGACCAGTAATCGTGGCCCAGCCCCTCTCGGCAGACGCTTCATGACAGGAAGGACTGAAATGTCTC
TTGGACGCCTGGTCCTTCCCTGATGTTCTCGCGGCCGCCGGTTGGAGCANANATGGACACCCCTGGTCT
TTGCCTGTGCGTGCGTGTGTGCNTGTGTGTCCCCCCACGCTGGTCCTGGTGCCTTCCCCATCCNTCATN
AACATGNTNTNACCAGCCTTCCCCATGTTATNCATTTATCTTNNNGCNCCTNGGGNGGNCCTANNNTA
GCTTCTTTTNNGGGGGAGCCNANANGNTAAANCGANGGTNANTTTCAAATTTCANNGNGATGGAACA
ACTCANAANGCAATAAATGGNGATGAGGTTNCCCTGNTNNAANNTNTGNNTTNTNTNGGGGGNCCNN
TTCCCCAANTGGCCAAAANTTTGGGGNNGGNNTNAANNNNNNNNNNNNNNNNNNNNNNNNNNNNNNN
NNNNNNNNNNNNNNNNNNNNNNNNNNNNNNANNNN
TTNTTGAACNANNTGNNTGGGANCCTCCNACCGGCGGGGGNGCGGCCGCTCTAGAACTAGTGGATCC
CCCGGGGTGCAGGAAAAAAAAAAAAAAANTTTTGGGGGGGGGNCCNNGGNNCCCAANNTCCNNCGG
GGGNGCGGNTTNAAAATANAAACNTGNNCTAANCCAAATCCNNNTATTTNNANTNNTTNNNNTAANA
GGGGATTANTCCNNGNNAGNANTCTCCTNTTCNCATTNATANACNANTCCNNTCNNTANTATTNTAAT
NTNNAAAACTTTANAATAANATTGNNAGNATTNGNNTTATTANCTTNCTCNATNANCNANNATTTTCNA
NNCANTAANANTNTATCTTANTTTNTNNANCTTATNANCTNTATTCTNAANATNANNANNNAGGANNA
TAGGNNCAAAGNNTCTCNNCCNNTANNTAGNGCGANNCCTNNCNCANNCNAAGGATNGNCNTTTNNN
NCNGANNTANNGTNGNNCNNGCNNTGGNGCAATTAANCCAAANTNACAANCTCATGGTNGGNGGGTG
TTNCCNACGGNTGACTAACCCTGGNGCCANTTTCTCTANTGTCNCCCAGCNGGNACTNNGTGGNANGT
NTCCNNTGNNGTNTTGNANCACACNNACCCGTNTACCNTGACTTNAAATGGNTGGAATTTNCGGAAAC
TGNACTNAAAGNGANGGCCCACCCCNGTNGANNANCNTNNATCGCCNTCAANNCCCCAAAANGTCAA
NNCATNCCTTTNCAANANCCCAANGCCTCNAACCTANCCNNNCANTGNNTACTAAAANTTAATAAACA
ANNTTCCTNNNATNANACCTTANTAATCCNNCANANGTTTNNNNGCCTNNAANGNTTTATCCGCGNNN
NCCNTTNGNATTTTNATTGNTNTCTNCCNNACATNNTNNCAANTAATCNATCNCNNTTANCNCNNCTN
CTGANNNACCAATNTNCNANNNNCTTGTTNCCNATTNNACCTNCATTTNNCNNNCAATCACNGATCNN
NACNGCAATTTCNNAAACCNNTANAANTNGNGANTTGGGNCNCNCCCCNTCNCCNCTTTCTCACTCNT
NTCTANATAATNAANNATNNCACCATCTACCAACTTNCNCTNCCCNCCCANNCCAAGGNGNGCCCCTC
TTTANTTNTNTTTNNNANCNTANTNTCANCATANNTACTNATATNNAATTCC
TTTGANNNCNNTTGGAGNTNNNCTACAGGGGCNGNCGCTCTAGAACTAGTGGATCCCCCGGGCTGCA
GGGNGAAAANNACCNTTTAANNCTAAGCNAACATGGGNAANGTNNTCACCCACATGAACNTCNTTGNN
ATTGNGCNNGTAAATTNATGGANGTCTACNACNACNGANCATATGNTCTANAAACNANGCTGGATCT
ATAAGAANCNAGCTGGGTCTATGANAAGTGGNCTTAACATGTATGACCACTTTTTTAANCAGCCANAT
CACAATGAAAACATCACTACTGTAATGCTTGGCCCATGATGTTATTTCCTCACTATCAGTTGAGACCCA
GCAATAAATATAAAACGTTGCAAAAAAAAAAAAAAAATCTGNGGGGGGGGGCCGNGNTNCCANTTTN
CNNNTANGGGNNNGTNTTTNNNNGNNTGGGCTTNNCCTCAANTTNTGCTGCTTNTTNNTGATGAAATT
TTGAANTCCNNNTNTTTNCTNTCGAACNNNGCAATANCTGTTTNATNTTTCCNNTTTNTGNGCTTTCCTN
TTCTCAGTTTTNTAAACATTNNTANTNTTNNNGAGCTTTTANCNCNNATGNNTTTTCNCCNGGNTNACN
CTNNCNNTTTGCCTNATATTNTTATNCANTNTCNCCTTTTTTTCTNGAACTTTTCCCNNGNCTTTTTTCTT
NAANNANNATTNNNCTNTNTTCCTNCTTTTNTATTTNATTNTNTTNNTTTTNCTAATTTTCTTNTCNTTA
GANTTNNGCNTTANAACNATTCTTNNAACCCTNCTTNTTNTCAATTTTACNNNNNTTANTTNNTNCTGG
ATTTTTTAATTTNCCTCTCNCTNTNNGACTNGNNTANNTTTTCANACTTGNANANTNTCNTCTNTTTTNN
ATACNTNTTNCGTTTACCCACTANNGTTCATTCCNTGTTTNTCNATTCTNTNTTTANNTANTCNNCTATG
NTTNTNNNGANNTNTCNTNANNATTNGNANNTNTTTATTTNATNNTTTTCCCGNNTTTTATNTATTCTT
TNGTGNNNNCNN
AAANCTGGAGCTCCACCGCGGTGGCGGCCGCTCTAGAACTAGTGGATCCCCCGGGCTGCAGGAATTCG
GCACGAGGAAGGTGCAACTTTCTTCGGTCGTCCCGAATCCGGGTTCATCCGACACCAGCCGCCTCCAC
CATGCCGCCTAAGTTCGACCCCAACGAGATAAAATGTACCTGAGGTGCACCGGTGGGGAAGTCGGTGC
CACGTCTGCCCTGGCCCCCAAGATCGGCCCTCTGGGTCTGTCTCCAAAAAAGGTCGGTGATGACATCG
CCAAGGCAACTGGTGATTTGGAAGGGTCTGAGGATTACAGTGAAACTGACCATTCAGAACAGACAAGC
CCAGATTGAGGTGGTACCTTCTGCTTCTGCCCTGATCATCAAAGCCCTCAAGGAACCACCAAGGGACA
GAAAGAAGCAGAAAAACATTAAGCACAGTGGAAACATTACTTTTGATGAGATCGTCAACATTGCCCG
GCAGATGCGGCATCGGTCTCTAGCTAGAGAACTTTCTGGAACCATTAAAGAGATCCTGGGGACCGCCC
AGTCTGTGGGCTGCAATGTTGATGGCCGCCACCCTCATGACATCATTGATGATATCAACAGTGGGCGC
AGTGGAGTGCCCCGCTAGTTAAGAACTGCAAAGGAAAATAAAGGACCATTTGACAACCAAAAAAAAA
AAAAAAAACTNGGGGGGGGGCCGGGNNCCCAATTCCCCCTATAGGGGGGNNNNNNNNAAAANNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNC
AAANCTGGAGCTCCCCGCGGTGGCGGGCGCTCTAGAACTAGTGGATCCCCCGGGCTGCAGGANTTCGG
CNCGAGGCAGAGGTCTTTAGAAGAAGGAGAGGGGCCAATAGCTGTCATCATGACTCCAACTCGAGAA
CTGGCTTTACAGATTACTAAAGAATGTAAGAAGTTTTCAAAGACCTTGGGCCCTATAGTGAGTCGATT
ACAGNNGGANCNGGANTCNNNGANCNNATNGCTGNCTGAAAANAGGNGCTGAANTTATTGNTNGNCC
NCCTGGNCNAANGATTGNCNTGTNNNCANNTANTANNGGNGNGNNTACNANNTTTNNAANAGNGAGN
NNTGNTGTTTTANANGAANCNNACNNAANGTNGACTGGGNTTNGANCCCANGNNNTGCNCNTTGGGG
ATAANGNNCNCCCTNNCNNACNNACNNNTNTGNTTTNNNCTNCTTTCCCCANANNTATGGNACCTTNG
NTNNGANAANNCNNNNNNNCCCNTNNNNNNCNNGTNGNNGCNNAANNNNNNNNNNTCNNNNGNGN
NNCNCNNNNNANNGNNNNNGANNAANAANANAANTTNTNGNNCNNTTNNNTTNNNNNNNNNTTNCN
ANAANNNNGNNNNTGNNNNTTNTNNNNNGNGGNNNNNNNNNNACNTGCNNNNGGNNNNCNNNNNN
ANNNNNNNNNANNNNNTNACCNNTNNNNNNNNNTTNNNNNGGNNGNNTNNNNNNNNNNNNCCNNN
NNNNNNNNNNNNNNNNNNNTNANNNANNGGNNNCCNNNNNNNNNNNTTNNNGNCCNCNNNNNNNN
NNNNNCCNNNNCCNNNNNNNNNNNNNNNNNNNNNNNNNNNNNNNNNNNNNNNNNNTNNNCCNNNC
CNNNNNNNNNNNNNNNNNNNNCNNNNNNNNNNNNNNNNNNNNNNNNNNNNNNNNNNNNNNNNNNN
NNNNNNNTTTNNNNNNNNNNNNNNNNNNNNNNNNNNNNNNNNNNNNCCN
NCCNTATCTGGAGCTCCACCGCNGTGGCGGCCGCTCTAGAACTAGTGGATCCCCCGGGCTGCAGGAAT
TCGGCACGAGGGTCAGNNAACATCACCGAGCTGAATCTGCCGCAGCTAGAANTGCTCAAGAACCAAC
TGGACCAGGANGTGGAGTTCTTGTCCACGTCCATTGCCCAGCTCAAGGTGGTTCAGACCAAGTATGTG
GAAGCCNAGGACTGNCTGAACGTGCTGAAGAAAAACANCGAGGGGAAAGAATTACTTGNCCCACTGA
CGAGNTNTATGTATGTCCCCGGGAAGCTACNTGATGTGGAACATGTGCTTATTGATGTGGGAACTGGN
TACTACGTAGANAAGACNGCTGAGGATGCCNAGGACTTCTTCAANAGGAANATAGACTTCCTTANCA
ANCNAATGGAAAAAATCCAACCAGCTCTNCAGGANAAGCACNCCATGAAACANGCTGNCATGGNGAT
NATGAGCCCNAANATTCANCAGCTCACAACCCTGGGAGCAGCTCAGGCTANCGCCAAGGCCTGAGAG
CTTGTTTCACAAATGGGGCAGAGGGTGCCTGCTTCTGGGGCCTGGGACTTTGTGGACANTGGTTTCCTG
GAATGGGGAAANAAAAAAGGTCTGTGGTTTAATGGTAATAAAATGAGGCNNCTGGGCACANTANTCN
NAGGTCNAAATTNTCCCNCNTNGGGGGGGGGNCNGGGCCCCCAATTTNCCCCTTNNTNGNGNGGNNCC
CNNTAANNNNNGANANGGNCNNTCNTTTTTTTNNANTGNTNNNNNNTTNNNNNNGGNNNTATNNGNC
TTNTTNTNNAAACCCTTNNAACTTNTGNNTTCTTTCNNNGTTACTNNTTNNGNNGGNNNTTTNNTTNGT
NTTTCCCCCCNNNNTNTTNNNNNNGNNTNNGNNTTTNNNNNNNNTATCCNNNNNANNNTTTTTNNTNT
TTTTNNTNNC
GNAANACACNTATTNNTTTTTTTTTNNNNNNTNTTNTCNCTCCCNCNNNNCANTNNTTCGTNANCNC
NTNTTCNNNGGGANANNAGNAGATNANAACTTTGAGCNCNCTGGANTGCANACAGCGGNGGCCGCTC
TANACTAGTGGATCCCCCGGCTTGCAGGNANTGNAAATTGTAACGANTNNNNACCNATANAANNAAN
ATTGGCTNCGGGGGCCCNACNCANTATNTATNTTGTGCCANNTCNTGGGTTCGTAATGNTGACTTTCA
TGCTANTAANCTTNGNTGCTANTNTGAGCATGGCANAANGGGGATNGNTAAGTCACCCNTAANGNAC
NNGNNATTNCNNTTTGCAAATANTCTTGCTNGANANCTTTGGATTGCATNTNTGNTAANCAAANNTCN
NTTCATNTTGANANACCGTTTGCCNNTNGNCTTCTCCTNNGCATGGTCTACTATAANATNTNNTGGGCA
NTTAGGNNACCNGTCGCCTTGCTNGNNATCCNNCATTGNACACTTGNTNGTTNTCANAATCCTTGNGN
AAATGGNTGGANCNNCCTATANGCTNCNAAACTNAAATTGANANNNATNCTGNNANNAGAAAGNNN
NTNGTNANANANNTNCACTCNTTNNANGACANNNTNNNCNTATCGAAANNCTCTNTTTNTCNTGGNTT
ANCAATTCGCGNNTAGCNTNNTAANTNNNTTANAAANNGNGTNACTANTGACAAATNNNNANTTTNN
NGNNTTNCAGNCNAATCGNNGCNNTTTGNGTTACCCTTNNACTATNATNATTGTATTTCAACNACANN
TNGTANANNACNTNCTTGGCTTCTTTCACATGCTTTCTNNAGNTGNNANTCATTTTTTTGANACTNATC
NNCAATGNANTANATTANANTTCNCTGNNTATTTTNATNAACNTNGATGNGTNNGCCATTGNNTAACN
CTAGTNACCTANANACTTGGTATNATGACAATATTAATNANCNCNTCNCTCTTGNCNCNGNNTTGNAA
TTAAAACAANTATGTNTGTNANNNCNTACTCGGATAATNCNTACTNGTGGNTAAACANAATTGGNGCC
TTTAANATTNTATANTTNNAATAACTCCNANGTNNGNCNCANNCTTNTTANANTATGNNGNTAGANNT
ANTNGGNTNANTTNCNTGCNATNTANGNTCTCNCTNNCTNTNGTNTNTAATCNACNAGACATNCNTTNT
CTATNTNTTATNNAANNGCCCTANCTNTCTCNTNGTNNCCACNANTAACTNACANNATGNANCTTGTN
TCTATNTNCTGANCTCACTATCGANNNANTTATACCTNTNTCTTTCNCTCTACNATATTACTANCTANN
TCNCNNNCNNGNTCGNANANAACAATCGNGGTCACTNTNTCGNNAANNTCTGCTGGGGACATATNNG
TCG
GNAANACACNTATTNNTATTTTTTTTTNNNNNTNTTNTCNCTCCCNCNNNNCANTNNTTCGTNANCNC
NTNTTCNTNNGGGANANNAGNAGATNANAACTTTGAGCNCNCTGGANTGCANACAGCGGNGGCCGCTC
TANACTAGTGGATCCCCCGGCTTGCAGGNANTGNAAATTGTAACGANTNNNNACCNATANAANNAAN
ATTGGCTNCGGGGGCCCNACNCANTATNTATNTTGTGCCANNTCTNTGGGTTCGTAATGNTGACTTTCA
TGCTANTAANCTTNGNTGCTANTNTGAGCATGGCANAANGGGGATNGNTAATGTCACCCNTAANGNAC
NNGNNATTNCNNTTTGCAAATANTCTTGCTNGANANCTTTGGATTGCATNTNTGNTAANCAAANNTCN
NTTCATNTTGANANACCGTTTGCCNNTNGNCTTCTCCTNNGCATGGTCTACTATAANATNTNNTGGGCA
NTTAGGNNACCNGTCGCCTTGCTNGNNATCCNNCATTGNACACTTGNTNGTTNTCANAATCCTTGNGN
AAATGGNTGGANCNNCCTATANGCTNCNAAACTNAAATTGANANNNATNCTGNNANNAGAAAGNNN
NTNGTNANANANNTNCACTCNTTNNANGACANNNTNNNCNTATCGAAANNCTCTNTTTNTCNTGGNTT
ANCAATTCGCGNNTAGCNTNNTAANTNNNTTANAAANNGNGTNACTANTCACAAATNNNNANTTTNN
NGNNTTNCAGNCNAATCGNNGCNNTTTGNGTTACCCTNNACTATNATNATTGTATTTCAACNACANN
TNGTANANNACNTNCTTGGCTTCTTTCACATGCTTTCTNNAGNTGNNANTCATTTTTTTGANACTNATC
NNCAATGNANTANATTANANTTCNCTGNNTATTTTNATNAACNTNGATGNGTNNGCCATTGNNTAACN
CTAGTNACCTANANACTTGGTATNATGACAATATTAATNANCNCNTCNCTCTTGNCNCNGNNTTGNTAA
TTAAAACAANTATGTNTCTNANNNCNTACTCGGATAATNCNTACTNGTGGNTAAACANAATTGGNGCC
TTTAANATTNTATANTTNNAATAACTCCNANGTNNGNCNCANNCTNTTANANTATGNNGNTAGANNT
ANTNGGNTNANTTNCNTGCNATNTANGNTCTCNCTTNNCTNTNGTNTNTAATCNACNACATNCNTTNT
CTATNTNTTATNNAANNGCCCTANCTNTCTCNTNGTNNCCACNANTAACTNACANNATGNANCTTGTN
TCTATNTNCTGANCTCACTATCGANNNANTTATACCTNTNTCTTTCNCTCTACNATATTACTANCTANN
TCNCNNNCNNGNTCGNANANAACAATCGNGGTCACTNTNTCGNNAANNTCTGCTGGGGACATATNNG
TCG
TTTNAACAACAATCTGGAGCTCCACCGCGGTGGCGGCCGCTCTAGAACTAGTGGATCCCCCGGGCTGCA
GGAATTTCGGCACGAGGGCGGTGACGGCCGAGGCGACAGAACCGAGGGGCTGTCGTTGGTGGGCGCTC
CAGGGCTCTGAACGAGAGAAGCGTGACCCGGAAACGGAAGCGAGTCCCCATCCCAACTCCGCCAGGT
TTCACGCTAGCCCCAGGCTTACTGGTAGGCTCCTGCTGCCATGGACCTGTTGTTTGGGCGGCGCAAGAC
GCCAGAGGAGCTGCTGCGGCAGAACCAGCGCGCCCTGAACCGGGCCATGCGAGAGCTGGACCGTGAG
CGACAGAAGCTCGAGACCCAGGAGAAGAAAATCATCGCGGACATCAAGAAAATGGCCANGCAGGGC
CAGATGGACGCGGTCCGGATCATGGCGAAAGACCTGGTGCGCACCAAGCGGTACGTCCGAAAGTTTG
TGTTGATGCGGGCCAACATCCAGGCTGNGTNCCTTAAGATCCAGACACTCAAGTCTAACAACTCAATG
GCACAAGCCATGAAGGGCGTTANCAAAGCCATGGGCACCATGAACAGACAGCTTGAAGTTGCCCCAN
ATNCAAAANATCATGATGGAGTTTTGAACCGGCAGGCAAAAATTATTGGACCTTGAAGGANGGAAAT
GATTAACGAACNCCCTTTNNTGACNCCATTGGGGGAACCAAGGACCAATTAAAAAGGAAAAGNGATT
GCTTTTTTAACCCCCAGGNNCTTGGGACCAAATTGGGGGTTGANNCCTTGAAAAAANTAAACTTGGT
CNAAACCTTTNCNTTTCCCCCGGGNGGGTTTCCNTTAAACGTTGGGNTTGCCCGGGGGAAAAAAAAA
NCCNAAGGNTTTCNGNGCTTTTGNCCTTGGGGGGGACNCCAAACCCCNTNAACCTTTGGAGGGAAAAG
GGTTTAAAAAAACTTTTTAAAAGGGAATTGAAATTGGCCCNNGCTTTTTNCCAAAGG
NAAAACTGGAGCTCCCCGCGGTGGCGGCCGCTCTAGAACTAGTGGATCCCCCGGGCTGCAGGAATTCG
GCACGAGGNCTTCNACCTGANNCCTGCCAACNNCTNCTACCNCTNCCAGGCTCTNCACNACNNCTACC
NCGNCTGCATTAACGNNAANGGGACCTNTNCGGCCCTGGACNNGGTCNTGNNCCNCNTGNCAGTCAG
NNGGNNNNNGGGGGACCAGNTNNNTNCATTCCATNNNNACNCTNCACNCTTCACCGNCATGCTTGAC
ATGGNTCAACACTNACNTGTGNANGTTCTCNCNCATCGAGANCTTNTGNNACTGAGNTACTCTNCTGC
TGTGTTGCNTTNNGCCCCNGTTGACCCCAGAGNNNACCNNNCNACTGGCNGAAACCCTGCCCTCCTGC
CTGCNTCNTGCGCNNGGGACGNGGGTNTNAAAAGACATGCTGGAGCCNGGATGGGGACGTGGCNTGG
CCNCACGTTCACTGTCNNCNGGCTGNCCTCTATGNTNGGGAACGGAACCCCAAGNGGCGGCGGGACT
GACTCTTTCCCCTGGCTGGGNGATGCANACCATGANTGCGTNTNCATCTATNTCTGATGCNTCANTACA
ACATGCGTGCTGGTACCCGATTTTGGGCCCCGGTTGGTCCGGACGATATGCCTTTTAACTTACATCCC
GCACTGGGACTTTCCGAAGCTCTTNANAANTNANNNNTTNGGGNNNGNNANTTNTTCAATTCCNNANC
CCNNNCCNNANNGNTTTTTTTGNAAGGNNAAANCCCGGGGANNNCCNNCCGGGNGGAANGGNAANN
NGGNAACCCTTCCAANAAAACCAANGGTTNNCCCTTNCNNNAGGGNNNANGNNNNNCCNNNNNNNN
NNNNGGNNNNNCCCNNNNNNNNNNNNNNAGGAGTTTTGAAAACCCTTTTNTTTCAAGNNTTT
TTTGGAAANCAACAATTTCTGGNANGCTTCCCACGCGGCGGGTGNGCCGGGCNCGCTTCTTAGAAACT
AGGTGGGAATCCCCCCCCGGGGCTTGCAGGNAAAANAAANAAAAAAAAAATNTNTNNGGGGGGGGG
GGNCCCCGGGANCCCCNAANTTTCCNCCCNTTATAAGGGGANGTNCNNTNTTNAANAANNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NTNNNNNNNNNNNTNNNNNNNNNNNNNTNNNNNNNNNNNNNNNNNNNNNNNNNNNNNNNNNNNNN
NNTNNNNNNNNNNNNNNNNNNNNNNNNTNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNTNNNTNNNNNNNNNNNNN
NNNTNNNNNNTNNNNNNNTNNNNNNNNNNTNNNNNNNNNTNNNNNNNNNNNNNTNNNNNNNNNN
NNNNNNNNNNNNNNNNNNNNNNNNNNNNNNNNNNNNNNNTNNNNNNNNNNNNNNNNNNNNNNTNT
NNNNNNTNTNNNNNNNNNNNNNNNNNNNNNTNNNNNNNNNNNNNNNNNNNNNNNNNNNNNNNCTN
NNNNNNNNNNNTNNNNNTNNNNNNNNNNNNNNNNNNNNNNNNNNNNNNNNNNNNNNNNNNNNNNN
NNTNTNNNNTNTNNNNNTNNNNNNNNNNNNNNNNNNNNTNNNNNNNNNNNNNNNNNNNNNNNNNN
NNNNNNNNNNNNTNNNNNNNNNNTNNNTNNNNNNNNTNNNNNNNNNNNNNNNNNNNNNNNNNNNN
NNNNNNNTNNNNNNNNNNNNNNNNNNNNNNNNNNNNNTNNNNNNNNNNNNNNTNNNNNNNNNNNC
NNNNTNNNNNNNNNNNNTNTNNNNNTNNNNNTNTNNNNNNNNNNNNTNNNNNNNNNNNNNNNNTNN
NNNNNNNTNTNNNNNNNNNNNNTNNNNNNNNNNNNNTCNNNNNNNNNNNNNTTNTNNNTTNNNNNN
NGNNNNNNNNNNNNNNNNNNCNNNNNNNNNTNNNNNNNNNNNNNNNNNTNTNNNNNNNNNNNNNN
NNNNNNNNNNNNNNNNNNNNNNNCC
AGNCNATCTGGAGCTCCCCGCGGTGGCGGCCGCTCTAGAACTAGTGGATCCCCCGGGCTGCAGGGTGA
AAACCACCGTTAAACCTAAGCCAAAATGGGAAAGGAGAAGACCCACATCAACATCGTTGTCATTGGG
CACGTAGATTCAGGGAAGTCTACCACGACTGGCCATCTGATCTACAAATGTGGCGGGATCGACAAGAG
AACAATTGAAAAGTTCGAGAAGGAGGCTGCCGAGATGGGAAAGGGCTCCTTCAAATATGCCTGGGTC
TTGGACAAACTTAAAGCTGAACGTGAGCGTGGTATCACCATTGATATCTCCCTGTGGAAATTTGAGAC
CAGCAAGTACTATGTTACCATCATTGATGCCCCAGGACACAGAGACTTCATCAAAAACATGATTACAG
GCACATCCCAGGCTGACTGTGCTGTCCTGATCGTTGCTGCTGGTGTTGGTGAATTTGAAGCCGGTATCT
CCAAGAACGGGCACACCCGTGAGCATGCCCTTTTGGCTTACACCGTGGGTGTGAAAGAACTAATTGNT
GGCGTTAACAAAATGGATTCCACTTGAGCCACCCTATAGCCAGAAGAGATACGAAAGAAATTGGTAA
GGGAAGTCAGCACCTATATTAAGAAAATTGGCTACAACCCCGACACAGTAGCATTTGTGCCAATTTCT
GGCTTGGAATGGTGACAACATGCTTANAACCAAGTGCTAATATGCCATTGGTTCAAGGGATGGAAAGT
CACCCCGTAANGGACGGCATGGCCAGTGGGAACCCCCCTTGCTTTGNAACCTNTTGGATTGNANTTTT
GGCCACCAAATTTGGCCCAAATGGAAAAAACCCTTGGGGTTTGGCCTTTCCNGGAGGTTTATNAAAAT
GGGGGGGATTTGGGNCCTGNCCCTTGGGGGCNGG
AAANCTGGAGCTCCACCGCGGTGGCGGCCGCTCTAGAACTAGTGGATCCCCCGGGCTGCAGGAATTCG
GCACGAGGGCAGTTAACATCACCGAGCTGAATCTGCCGCAGCTAGAAATGCTCAAGAACCAACTGGA
CCAGGAAGTGGAGTTCTTGTCCACGTCCATTGCCCAGCTCAAGGTGGTTCAGACCAAGTATGTGGAAG
CCAAGGACTGTCTGAACGTGCTGAAGAAAAACAACGAGGGGAAAGAATTACTTGTCCCACTGACGAG
TTCTATGTATGTCCCCGGGAAGCTACATGATGTGGAACATGTGCTTATCGATGTGGGAACTGGTTACTA
CGTAGAGAAGACAGCTGAGGATGCCAAGGACTTCTTCAAGAGGAAGATAGACTTCCTTACCAAGCAA
ATGGAAAAAATCCAGCCAGCTCTGCAGGAGAAGCACGCCATGAAACAGGCTGTCATGGAGATGATGA
GCCAGAAGATTCAGCAGCTCACAACCCTGGGAGCAGGTCAGGCTAGCGCCAAGGCCTGAGAGCTTGTT
TCAGAAATGGGGCAGAGGGTGCCTGCTTCTGGGGCCTGGGACTTTGTGGACGTGGTTTCCTGGAATGG
GGAAAGAAGAGGGTCTGTGTTTAATGCTAATAAATGAGCCAGCTGGGCAAAAAAAAAAAAAAAAAA
AAAAANCTTGGGGGGGGGCCCGGGGCCCAATTTTCCCCTTTTGGGGGGGNNNNNNNNAANANNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNC
ANNNCTGGAGCTCCACCGCGGTGGCGGCCGCTCTAGAACTAGTGGATCCCCCGGGCTGCAGGCNANG
GNGTCAGAGCTGTACGACGTCACTTGGGAAGAAATGCGAGATAAAATGAGAAAATGGAGAGAAGAA
AACTCACGAAATAGTGACAAATTGTGGGAAGTTGGGAGAGGAATTAATTAGTGAATATGCTTCTAAGC
TTGGGAGATGATATTTGGATCATATATGAACAGGTGATGATTGCAGCCCTCGACTATGGTCGGGATGA
CTGGCATTGTTTTGTCTTCAGGAGTTGAGAAGACAGTTCCCTGGAAGTCACAGAGTCAAGAGACTAA
CGGGCATGAGATTTGAAGCCATGGAGAGATCGATGATGCTATTCAGCTATATGATCGAATTTTACAAG
AAGACCCAACTAACACTGCTGCAAGAAAGCGTAAGATTGCCATTCGAAAAGCCCAGGGGAAAAATGT
GGAGGCCATTCGGGAACTGAATGAGTATCTGGAACAATTTGTTGGAGACCAAGAAGGCTGGCATGAA
CTTGCAGAACTTTATATCAATGAACATGACTATGCTAAAGCAGCCTTTTGTTTANAGGAGCTTATGATG
ACTAATGCTTACAACCACTTATACTGTCAGCAGTATGCCGAAGTTAAATATACCCAAGGTGGACTTTG
AAAATCTGGAACTTTCAAGAAAGTATTTTGCCCAGGCATTTGAAACTTGACCACNGAAATTTGANNAG
CTTTGTTTGGACNTTTCATGGCTGCAAGNCATATTGCTTCTTAATCCCAAAGCCNGGNCCNAAACNAA
AAAANACCCCNTGGANTTTTGTTTTTTGGGCCCCCCNNCAAAAAACAAAANNTTNTTNNTTTTNNGGG
GNNNANNNNNAAAGGGANCCCCNTTCTTTTNTTNAGGGGNTTTNAAAAGTTTTTGNAANCCNTGNGNA
ANNCCCCCNNTTTTNGGGGGNAAATTNGNCCCCN
NNNNATCTGGAGCTCCACCNCNGTGGCGGCCGCTCTAGAACTAGTGGATCCCCCGGGCTGCAGGCAAC
TTCGGNGTGAGCACTGTGCCCGCTTCCTGGCTGACCGCTTTGTGGAGGGTGTGTGTCCCTTCTGTGGCT
ACGAGGAAGCCCGGGGTGACCAGTGTGACAAGTGTGGCAAGCTCATCAATGCCATTGAGCTCAAGAA
GCCTCAGTGTAAAGTCTGCCGGTCGTGCCCTGNGGNGAAATCCTNTNANCACCTGNTTCTGGACCTGN
CTAANCTGGCAGCGCGGGTGGAGGAATGGTTTGGAGAAAACNTTGCCTGGCANTGACTGGACTGNCAA
CGCCCGGTTCATCATTCGTTCTTGGNTTCNAGANGGCCNCAAGCCCCGCTGTATAACCCGAGACCTNA
AGTGGGGAACCCCTGTGCCCTTANAANGGTTTTGAGGACAAGGGGNTCTATGTCTGGTTTGATGCCACC
ATTGGCTATTTGTGCCATTACAGNCAACTACACANACCAATGGGAGAAGTGGTGGAACAACCCACAA
ACAAGTGAACCTGTNTCAGANTCATGGCCAAGGACAATGTNCCCTTCCATGGCATATANCTTTCCTTCT
TCANCCCCTATGAGCTGNGGACAANTACACCTNTNGNCAANGCACCTCATTTGCTTACANAAGAATTC
TTGAATTACNGAGGGANGGGGAAAATTTCTCTTANTAAAACCCCNGGTGTGGGGAGNNGTTTTGNGG
GACANTNTNCCACNNAACACAAGGGAATNNCCNGGNTTGACAATNNGGGNGGCTTTTTTATTNTTGCT
GGAACATNTNTTNCCTGAAGGGGGCAANAAAAAAANTGCCTTTTTTNNTTGGNNCAAAAAATNTTTNT
TTCNAAGAAANNAATTCCCCANCTTGNTTNTNAANCCTGGNTGNAAANTTTTTTTCATAAAANAAAA
CNGGGNANTGNTTNTGNNGNCCCCAAACTNNTTTTN
AACAAAACTGGAGCTCCACCGCGGTGGCGGCCGCTCTAGAACTAGTGGATCCCCCGGCCTGNAGGAA
ATTCGGCACGAGGCTTTTATTGAGACCACCCAAGGACCCCAGGGGACCCACACCTGGGGTNAGGGTAT
CCGGGGNTNTAANCCCNAAANNGGGGGNTNGTTTTCCNANCCCCNNGTTGNCCCNNCNANGGNNNAG
NCCCTTTTTTGNNCCNNGCNNGGAANGNAGNANTNANNNNTAAAACCAACNTCTTTNAACNNTCNA
NCCCAANNTTCCCAANCCNAAANCTNNAGGGNANTTGAAAAGAAATGAAAATGGGNTNGCACAATG
TGNCACNGAANTAGAAACCCTTTTTATAAAGAGATATTATAATATNGGGGCCCCATNTTCGNGNNCAG
GTTACANTTTGTGGNCAGGATAAAGTTGNGTGTGNCTANACCTGTGCCTGTGNACANCTCTTNNNGT
GANNNCACAAGATCNGAGCNCAGNNTNAAANNGCGCTCTNTTTTGTCACGAGAGNNCAAAGNCNNAN
GNGTGNCCACACAACACATANGNGTGNGNATGTGNACACACCACCAAGTGANANATGACACTNTGAT
AATATTTATAAAAAATATATTGTGTNTGTGTGNNAAAAAAAAAAAAAAAAAACTTNGGGGGGGGGGC
CGGGNCCCCAANNTTCCCCTTTTAGNGNGGGNNNNNANAANNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNN
AACAAAACTGGAGCTCCACCGCGGTGGCGGCCGCTCTAGAACTAGTGGATCCCCCGGCCTGNAGGAA
ATTCGGCACGAGGCTTTTATTGAGACGACCCAAGGACCCGAGGGGACCCACACCTGGGGTNACGGTAT
CCGGGGNTNTAANCCCNAAANNGGGGGNTNGTTTTCCNANCCCCNNGTTGNCCCNNCNANGGNNNAG
NCCCTTTTTTGNNCCNNGCNNGGAANGNAGNANTNANNNNTAAAANCCAACNTCTTTNAACNNTCNA
NCCCAANNCTTCCCAANCCNAAANCTNNAGGGNANTTGAAAAGAAATGAAAATGGGNTNGCACAATG
TGNCACNGAANTAGAAACCCTTTTTATAAAGAGATATTATAATATNGGGGCCCCATNTTCGNGNNCAG
GTTACANTTTTGTGGNCAGGATAAAGTTGNGTGTGNCTANACCTGTGCCTGTGNACANCTCTTNNNGT
GANNNCACAAGATCNGAGCNCAGNNTNAAANNGCGCTCTNTTTTGTCACGAGAGNNCAAAGNCNNAN
GNGTGNCCACACAACACATANGNGTGNGNATGTGNACACACCACCAAGTGANANATGACACTNTGAT
AATATTTATAAAAAATATATTGTGTNTGTGTGNNAAAAAAAAAAAAAAAAAACTTNGGGGGGGGGGC
CGGGNCCCCAANNTTCCCCTTTTAGNGNGGGNNNNNANAANNNNNNNNNNNNNNNNNNNNNNNNNN
NNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNN
TTNANCNCTATCTGGAGCTCCANCNCNGTGGCGGCCGCTCTAGAACTAGTGGATCCCCCGGGCTGCAG
GCTCGTGTGAGTGACATCGTCTTTAAACCCTGCGTGGCAATCCCTGACGCACCGCCGTGATGCCCAGG
GAAGACAGGGCGACCTGGAAGTCCAACTACTTCCTTAAGATCATCCAACTTCTGGATGATTATCCAAA
ATGCTTCATTGTGGGAGCAGACAACGTGGGCTCCAAGCAGATGCAGCAGATCCGCATGTCCCTCCGCG
GGAAGGCTGTGGTGCTGATGGGCAAGAACACGATGATGCGCAAGGCCATCCGAGGGCNTCTGGAAAA
CAACCCGGCTCTGGAGAAACTGTTGCCTCACATCGGGGGGAATGTGGGCTTCGTGTTCACCAAGGAGG
ACCTCACTGANATCAGGGACATGCTGCTGGCCAACAAGGTGCCAGCTGCCGCGCGTGCTGGTGCCATA
NCGCCGTGTGAAGTCACTGTGCCAGCACANAACACTGGTCTGGGGCCCGAAAAAACCTCCTTNTTCCA
GGCTTTAGGCATCACCACNAAAATNTCCAGGGGCNCAATTGAAATCCTTGAGNGATGGGCACCTGATT
AAAACAGGANACAAAGTTGGGCNCCANCNAAACCCCCCNTGTTGAANATGCTGAACATNTTCCCCCTT
TTTCNTTTGGGCTGGNCATTCCACCAGGGGGTTTGAAAAATGGGNGCATTTTACAANCCTTGAAGNGG
NTTGACATTCACAAAAAGGAAAACTTTGNANTTTCCCCCTTTCTTGGGAGGGGGGNCCCCCAATTTTTT
NCCCANCGGGTGNCCTTGCCAAANTGGGTTANCCCCAACCCGNNGCATTTTGGTNCCCCCNTTTTTTTT
ATTTAAANGGGGNACCAAANCNCNCTNCTTGGGTTTTTTTTTTGGGGGNNAACGGAATTACNCCCTTTT
CCCCNTTTTTTGNAAAAAGGGNCAANGGNCCT
NAAAGCTGGAGCTCCCCGCGGTGGCGGCCGCTCTAGAACTAGTGGATCCCCCGGGCTGCAGGNACTA
ATAANNANGGNNAACNATGCATTCATCGGACCTTNCAGCCCCATCAAACATTTCATCATGATGAAATT
TCGGTTCCCTCCTGGGAATCTGCCTAATCCTACAAATCCTCACAGGCCTATTCCTAGCAATACACTACA
CATCCGACACAACAAGAGCATTCTCCTCTGTTACCCATATCTGCCGAGACGTGAACTACGGCTGAATC
ATCCGATACATACACGCAAACGGAGCTTCAATGTTTTTTATCTGCTTATATANAGCACGGNAGGACTN
AGGTTTATATTAGGGTCCTTACACTTTCGNAGAACATGAAANATTGGNGTANTCCTTCTGCTCACAGTN
NTAGCCACAGCATTGTGNGGTCCTTCTACCATGAGGACAAATATNATNCTGAGGAGCAACAGNCATAC
CAANCTCTTATCAAGCAATCCCATACATCGGCACAAATTAAGTCCNAATGAATCTGAGGCGGATTNTC
ANTAANACAAGCANCCCTTTACCCGATCTTCGCTTTCATTTATCCTTCCATTATCATCATAGCAATTGG
CATAGTCCACCTACTATTCTTCCACGAAACAGGCTCCAACAACCCAACANGGAATTTCCTCAAACGTA
NACAAAATCCATTTCCACCCCTACTATACCATTAAGGACATTTTANGGGGCCCTNTNACTAAATTCTAG
CTTTTAANACTACTAAGNACTATTTCGCANCCCGACCTTCCTNGGANACCCAAANAACTACCCCCAAG
CCAATTCAATTNAANACACCCCCTTNAAATTAAAACCCGAGGGGANACTTTTTTATTGGCTACCNCAA
TTTTTACGAAACAATNCCCCAACCAACTTNGGNGGGNGTCCTTANCCCTTA
TTNANNAAAATCTGGAGCTCCACCGCGGTGGCGGCCGCTCTAGAACTAGTGGATCCCCCGGGCTGCAG
GGTAAATCTGTCTTTCCTTTTTGGCACATAGAATCTGAGACTGAAAATTGTNGAAGGAAAAGAGATGAC
AGCTGAGTTCTGGGTGGAGTTGATGAGCCCAAGGACTGGGCTGTTTGGGCCATATCCTGTTCATGGGT
TTTGCCACCACTATAGAACCTTCTGTGGTCTCCCTTGCGAAAGGGGCACAGCTCGTCAGCGTATTTAAG
GAGTGACCAGACTGGAGAGCATGAGGGGAACAAACAGGTGGGGGCTGTTATCCAGAGAACAGTGCTG
CTTTTCTGCAAGACCTTCTAGGGTAGTATAGAATGGTGTTGGGAGCAAAGACTTTAGAGTGAGAGAGA
GTCCTAACAGGTCTCTTCACTTTCTCTTTGGCCTGCAGGGATAATCCTACAGGTCCCTAATCCCTTATTT
GAAATCTTTTGGGATGGGTGGTGTTTTGGAATTCAGGATGATTCAGATTTTAGAAAGGGAATATAGTG
CCTTTATGATACATAAAGTAATACCCCAGTGGAGATTCAGGGCAGTGCCCTACTGGAATTTGAAATCTA
TCAATGTTTCTGTAGCTAAATATATGAATATTCNCACTAAGTGGAGATAAATAGATNTTATAAATAGG
ATCGGNGACGGGTCAAAATCAAAATTTTGGGTCAAAGTTATGNAACAAACCTATTGTTTCTTTCANAA
ATTTTTTTTATTTTGGAAATTATAAAATAAGGGAATTATGGGCCTTAATAATTGGTNCCCCAAGGGTGG
TGGGGGAAGGNAATAATGTGNCCCAAAAAAGCCTTTTTAAACCNCNCCCCCATTGGGAAGGGATTTGT
ATGCCAATTTTCAATTCCCAAAACAAAAATACCAAATGGTNCCCAAGGGGGGNGGGCCTTTTTTGNAC
CTTACAGGGGGNCCAATGGTNCCGG
NNCAANNCTGGAGCTCCACCGCGGTGGCGGCCGCTCTAGAACTAGTGGATCCCCCGGGCTGCAGGAA
TTCGGCACGAGGCTTTCCTGCAAAGTAGGGCCACACAGAATGTTGCAACTACCGGAGGTTTTTTTGAT
NANAATNTGACTNGCAGNCTTAAGCTTCTAAATTNCTGATTTAGTTGAATCTTGGTGAGAACCANAGG
CCGGAACTCAGCTGCCCCAGGACTGTCCAAGGAGCAGGAGCAAGTGGTGGCCCTGAACTGATGGGGT
GCCCGGAAAGCATCTGTGGCCAGCGTGCTGGGGTTAACAAGACCTTGGTCATCCACCGAGGAAAGCA
GGAAGTTGTTTCCAAAACAAGGAGGAAAAAATAGATGCTGAAGAATCAGAAGCTACAGCTGTGCAGC
ACAGGCTGCCCTCAGACCTGGATGGACATAGCCCAAGCCCCAAGACGAAAAGCTTTCTGTGATACACT
GACATGTTTATAACTGTCCGTGATCTTGGGGGCAGGCACCAGAATTCCTCTGTCTGTTGGAGAAAATA
GGCATAGAGACCTTGGGCCAGAATGCTAAACCCGGAGAGCAGGGCGTGAGCCACTCTTGCAAGAAAA
AAGTGAGAAGGACGTGTGGGCTTNCATCTTGGGCAGATGGGANAATGCAAAACAAAAAAANCGTGGG
GAANANATGGGANCCANGCNAACNGGAAGGCTGANGGCATGACAAGGNCAGTGGGGCTTCCCAGTG
GAGGGNCCTTAAANGGAGTCTGANTACCTAACTTGNCCCATTAAACCCCTTTGTCCGTTCCCAGCCCCN
AACACCTTATTAAAAGTTAATTTTTTACNCCCTGNAAAAAAAAAAAAAAAAAAACTTGGGGGGGGGG
CCCGGGCCCCCAATTTNNCCCTTTTTGGGGGGNGGGGG
AAANCTGGAGCTCCACCGCGGTGGCGGCCGCTCTAGAACTAGTGGATCCCCCGGGCTGCAGGAATTCG
GCACGAGGTGACCTCCACCCATTGTCTTGGTGGCACATGGGTGGAACACTTGATCTGCTCGAGTCTGC
CTTCAACACACATTGCATCTTCAGATTTTCTACTTTTCTGTTCAAAACTAATATTCACCAAGTCAGACTT
TGTGTTAATTTTATATCAGGGTATTGGCTGCCAGGGGGTCATCCCTAAGTGGCCTGAAGATGGACAAA
GGGAAGTAACAGGCACGTGATGTTGGCAAGGATGCTTCTAGGGCTAGAGGATCAGTGGTGGGAGAGA
GCTGCAGAATCCAGCAGCCAGAACTGCAGATAACGAATCTTATGGTCAGGGGCTGTGACTGAGAGAA
GGAAACTGAGGTTGTGTTCTGAAAGTACATAAACTCTCACATATACCCAGTTCTTCACCATCTCCCCTC
CTCACTTTGCAGTGCCATTTCTTTTTGCATTAGGCAATTTGCTCAGACTTTCCAGAGCCATGGCGCATCG
GTTCTCTGGAATCCCCCACACCTCTGAGAGGTGGATCACCACGTCCTGCAGGGCTGCTCCCCTCCAAAC
TACCTTTAGGAGATGCAGGACAGGGAGGCTGTTTCAACCAGAAAGACCAAAATCAAGAACCGAGGAG
CAAAACGTGGTAAAACAGAAAAANGGCAGGTGGCAAATGGGTTTCTTTTGGGGTTTCTGGTTTCTTTT
TTCACATCNGGGGATGGCTGNCACCAANAAATCTTTCAGTCCGCTTACATGGTNCTTCTTNTTNTNCCC
TNCCCACTTTTTTTTTCTATTAATCAAAAAAAAATTTCAAAAGCCAATNGGGATGGGCCGGATGTCACA
GGCTGGAAAAACTCGTTCCCCTCCAACCATTTTATTGAAAAAAGCTGGTTNNTTATTAATNATTGCCAAA
CCCTTTGGCCGGGGGNGGAAAANATTTGACNAATNTTTTNAAA
NAAANCTGGAGCTCCACCGCGGTGGCGGCCGCTCTAGAACTAGTGGATCCCCCGGGCTGCAGGATTTC
GGCNCGAGGGAAGCACGTCGTCTTCATCGCCCAGAGGAGAATTCTGCCTAAGCCAACTCGAAAAAGC
CGTACGAAAAATAAGCAGAAACGTCCCAGGAGCCGCACTCTGACCGCTGTGCACGACGCCATCCTGG
AGGACTTGGTTTTCCCAAGTGAGATCGTGGGCAANAGGATCCGCGTGAAGCTGGACGGCAGCCGACTC
ATAAAGGTCCATCTGGACAAAGCTCAGCANAACAACGTGGAGCACAAGGTTGAAACGTTTTCTGGTGT
CTATAAGAGCTCACGGGCAAGGATGTTAATTTTGAATTCCCAGAGTTTCAGTTGTAAACAAACGACT
AAATAAAATATTATTCTCAAAAAAAAAAAAAAAAAAAACNTGGGGGGGGGCCCNGNCCCCNATTNCC
CCTTATNGGGNGNNTNTAANANNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNTNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNGNNNNNN
NNNNNNNNNNNNNNNNNNNNNNNNNNNNNNNNNNNNNNC
AAAAGCTGGAGCTCCACCGCGGTGGCGGCCGCTCTAGAACTAGTGGATCCCCCGGGCTGCAGGAATTC
GGCACGAGGGGCTTCCAGGGAATCCCTCTGTTGAGGACAACTGGCTGTCTTCTCCTGCACCCTCCCCCA
AACAACCCTCAACTGCTAGACCCACCATGAAACCACTGGGTTCTGGGTCCCAGCTGCTGATTCTAGAA
CCCCGCCTCTGTGCTGCATCCTGGTCCCATCCCCTANATCTAGAACTCAGCCCACTGGATTCTANAACC
TCCATATTTACCTCACCTCGAGGCTCTTCCATCCCCAAGCTGTGCCCTGTCGCCAGCTTTGGTGAAAGA
NAGGGGCCCCTTATGTGTGCTGTGTTGTGTCTGCTGCACTTGGTTTGCAGTTGGGAGCGGAAGGCAGA
AGGGGTGTGATTGAAGTGTGTCCAGAGATGAAAAAATACATATATATTTAACAAAAAAAAAAAAAAA
AAAANNNGGGGGGGGGNCCNGGNCGGNANTTCCCCNTATAGGGNGNNGGATNAAAANNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNC
TTGAAAACANTGGACTCCACCGCGGTGGCGGCCGCTCTAGAACTAGTGGATCCCCCGGGCTGCAGGAA
TTCGGCACGAGGCTTTTATTGAGACCACCCAAGGACCCCAGGGGACCCACACCTGGTGTCACGGTATC
CGTGGGTCTCAGCCCCAGAATGGTGGGCTGCTTTTCCCAGCCCCCTGCTGTCCCCACCAAGGGACAGGC
CCCTCTCTGTGCCCTGCCTGGGATGGAGGACTCGATCATCAGATACGAGCTCCTTTGAACATCCAGCCC
CAGTCCTCCCCAGCCCAGAGCCTGAGGGTCATTTGAGAGGAAGTGAGAGTGGCATGCAGCAATGGAC
AGGACTCAGAAGCCCTTTTATAAAGGATATTTAATATCGTGGCCCCAGCTTCGTGGCAGGTTTCACTTT
TCTGGCCAGGTTAAAGTTTGGCTGGGCCTACGCTGCCCTGAGCAGCTCTCTGACTGTAACCACACGAG
CTGAGTGCAGTGGATGAATGCGGCCTCGTTTCTGCCAGGAGTGCANAGTGCCAAGCGTGGTCCANACA
CGACAGATGCCGTGTGCAAATGTGGACNACGACCCAANTATGACATATGACTCTGNAATATTTCTAAT
NNANATATTTTGGTTTCTNGGCTGAAAAAAAAAAAAAAAAAAAATTTNTNGGGGGGGNCCCCGTNAC
CCNTTTTTCNCCNTTTTNTNGNNNCCTNTATAATNNNGNNNNNTNNNTCTCCTCNNNNTNCTTCCCTNT
NTCNANTGNTCTNNTCTNNTNCNCTTTNCNNCNNCTNCACTNCNNNTTTNTNCNNTNNCNNNCTNCTGN
NNNNNNTNNTNGNCTNGTNNNCTCNNNTNNTNNGTCGTTNNTNGACTNCTNNNCTTTCTTGGTTNNNC
CNANNTNTNCTCTNCNNNTNNNNCNNNCTCNCCTNTNCNNNNATTNTNNTNCNNNTCNCNNNTCTNTC
TTNNCTTNNNNNNNTTCTNNTTTATNNCC
TTTGAAAACANTGGACTCCACCGCGGTGGCGGCCGCTCTAGAACTAGTGGATCCCCCGGGCTGCAGGAA
TTCGGCACGAGGCTTTTATTGAGACCACCCAAGGACCCCAGGGGACCCACACCTGGTGTCACGGTATC
CGTGGGTCTCAGCCCCAGAATGGTGGGCTGCTTTCCCAGCCCCCTGCTGTCCCCACCAAGGGACAGGC
CCCTCTCTGTGCCCTGCCTGGGATGGAGGACTCGATCATCAGATACGAGCTCCTTTGAACATCCAGCCC
CAGTCCTCCCCAGCCCAGAGCCTGAGGGTCATTTGAGAGGAAGTGAGAGTGGCATGCAGCAATGGAC
AGGACTCAGAAGCCCTTTTATAAAGGATATTTAATATCGTGGCCCGAGCTTCGTGGCAGGTTTCACTTT
TCTGGCCAGGTTAAAGTTTGGCTGGGCCTACGCTGCCCTGAGCAGCTCTCTGACTGTAACGACACGAG
CTGAGTGCAGTGGATGAATGCGGCCTCGTTTCTGCCAGGAGTGCANAGTGCCAAGCGTGGTCCANACA
CGACAGATGCCGTGTGCAAATGTGGACNACGACCCAANTATGACATATGACTCTGNAATATTTCTAAT
NNANATATTTTGGTTTCTNGGCTGAAAAAAAAAAAAAAAAAAAATTTNTNGGGGGGGNCCCCGTNAC
CCNTTTTCNCCNTTTNTNGNNNCCTNTATAATNNNGNNNNNTNNNTCTCCTCNNNNTNCTTCCCTNT
NTCNANTGNTCTNNTCTNNTNCNCTTTNCTNNCNNCTNCACTNCNNNTTNTNCNNTNNCNNNCTNCTGN
NNNNNNTNNTNGNCTNGTNNNCTCNNNTNNTNNGTCGTTTNNTNGACTNGTNNNCTTTCTTGGTTNNNC
CNANNTNTNCTCTNCNNNTNNNNCNNNCTCNCCTNTNCNNNNATTNTNNTNCNNNTCNCNNNTCTNTC
TTNNCTTNNNNNNNTTCTNNTTTATNNCC
TTTNNNAAANNCTGGAGCTCCACCGCGGTGGCGGCCGCTCTAGAACTAGTGGATCCCCCGGGCTGCAGG
CTTTGCTGAGGGTCACATTGAGCTCCCAGCGACTTCAGGGGCATCTTTAGGAGTCAGCACTATGGCAG
AAGACATCCAGGCCAAAATCAAGAACTACCAGACTGCTCCTTTTGACAGCCGCTTCCCCAACCAGAAC
CAGACCAGGAACTGCTGGCANAACTACCTGGACTTCCACCGNTGTGAGAAGGCAATGACCGNTAAAG
GAGGTGATGTCTCCGTGTGTGAATGGTACCGGCGTGTGTACAAGTCCCTCTGCGCGATATCCTGGGTGT
CAACTTTGGGACNATCGCCGGGCAGAAGGCACGTTTCCTGGNAAGATCTGAACTGGNTCCATCCCACCT
TTTNTGTCCTCCGTCCTTCTCCCCAGGGTGGTGAAGGGGGACCGGGGTACATGGTGATCCCCACCNTG
GGATCCTGACTCATGGTATAACTAATAATAAATGCTCGTTGGAAACGTGAAAAAAAAAAANAANAAA
ANCTTNNGGGGGGCCCCGNANCCNATTNNNNCNTATAGNGGGNNNNTTANNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNCNNNNNNNNNNNNNNNN
NNNNNNNNNNNNNNNNNNNNNNNNNNNNNNNNNNNNNNNNNNNNNNNNNNNNNNNNNNNNNNNN
NNNANNNNNNNNNNNNNNT
AAAAGCTGGAGGTCCCCGCGGTGGCGGCCGCTCTAGAACTAGTGGATCCCCCGGGCTGCAGGCTANNC
TCGAGTTTTTTTTTTTTTTTTTTANGNGCNTNGNANTTTNTTTTTNGTAAAANAGTGATGAGNANTAATN
NGNCNAAANAGGNNNCCANTTTTTGTNGGGAAACCAANCCCCACCCACAGGGGACNNTTTTCNNGNA
CCNAACGTANTCCCCNCNTTTTCNTGCCNAGGANGGCNNTAANCCCCGNAAAAGGNCCCCCGGNNTG
AGGNTCCAGGNCCAAACGGCCACNCNTCCGGCCCCANGGGGCNTTTTTGNANAGGNGGTTCCNGGGN
ACGGGGAAAGGGNCTNTCAANTNGGNCGNGANCTGGAGGGTTAAGGNTNAAAANTGGNGCGGGTAC
AAAAGCTTTCNTACCCNTGNTTTTNAGGNGTTTNACTGAGTTCCTCCTTGGCCATCANCNACANATGCA
ATANCTTGCCCTNAAGNAACCGGATGNTCGCCCAACCNTGTTNACTTGCCATTTTTGTTTTAGNAACNC
CANCAGGNCCGGGNATTGGNAANCTNTTTCNTTGAATNAACNGGGGGGGNNAAAAAAACTTTCTATC
NCCCGGGAAAAAGNCCCCTTCNNTTGCCGGGGANGGGCCCNTGANCCCCGGAGGANANAANCCCCTT
GAGGGGGTCNNAATCAAGGTTCCGGGGGGGGCCCGGANCCCAATTNCCTTAAGGGGAGNNNNNAAA
ANNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
TNNNNAAAAAGCTGGAGCTCCACCGCGGTGGCGGCCGCTCTAGAACTAGTGGATCCCCCGGGCTGCA
GGTTTTGAACCATCTGTTGAACAAGCCAGATATAGCAGCTGCCAACAGAATGTGACTCTGGAACCCCC
TAAAGCCCAGGAGGTGAGCTCAGCTTGCAGACTGAGGCACTCAGCTGGCCCCCACGCTATGACGCAGG
GGGACAAGGAGAGCAGCTCCAGAAGCGTGGCCCCTGGTACGGCAGAGAGCGAAGCCACGCTCCANCG
GAAGCTCCGGCAGAAGCAGCTGCAGCAGCAGTTCCGGGAACAGATGGAGAGACAGCAGCGGGCCGCC
ACGTCTGCTCTGCCCGGCGGAAGGACCGACCAGGCGCAGCGGCCACCGGTGCCTGGTGCGAAGGCCAG
CCTCCCCAGGACCCACTCACCTCCAGAGCCACTCACGCAGAGAGACCTGCTCCCAGACAGTGTTGAAA
TGTGGGACATGGCTTTGGATGCAGAGGACAGCGTGCTCANGCCCTTGTTGAAACCAGAACCACCGCAG
ACCCCTGCCATCTCGGTCCTAAAGAACGACATGGAGACCCAGAACAGGATCCCACCAAGCCTTTTGCC
ACCAGAATNCACAGGCACAGTCTGGGACCCTGGCACCTTCCAAACTNCCAGCCTTAAACGAACAGACC
ACAAGGCGACTGNGATTCCTTNTAGGAAGATTCAGGACATGAAAAAAANGGAAACTGGGATCCCCGT
TAANCCCCAGGGTTAAGGTCACCCTCCTAAAACTNTTGGCCTGGAGGGGACTTTCCTTTTGGTCATGAA
AAAGAACTGCTNCTTGCCNTGAAGAAGGGGCCTTGGCTTTAAGGGCTTCCNCCTTGNTTTATTCTNAA
NNNNNGGGGAGGNAAAANNNNCNTTNNNAAAANAANGGGGGGGNAAAAANNNNNNTTTTTNNAAA
AAAAACCCTTTNT
TTNNNCACANCTGGAGCTCCACCGCGGTGGCGGCCGCTCTAGAACTAGTGGATCCCGCGGGCTGCAGG
AATNCGNGACNAGGCCTCGNTGNCCACCTGGCATTCCTCCGACTNCTCTCCAGNCGGGCCTCTCATAA
CATCACATATCACTGCGAGAATANCATTGCATACATGGATCATGCCANTGGGAATGNANANAAAGCCT
TGAAGCTGATGGGGTCAAANGAAGGAGAATTCNNGGCTGNANGAAANANCNNATNCACATAGNCAGT
TCNGGAGGATGGTNGCACAAAACACACTGGGGANTGGGGCAAAACAGTCTTCCAGTATCAAACACGC
NAGGNCGNNANAGTACCTATNGNACANATNGCNCCCTATGATNTCGGGGGGCCTGATCAAGAATNTG
GTGCNGACATTGGCCCTGTTCGNTTTTTATAAACCAAACTCTATCCAAAGTCCCAGAAAAAGCTGNAC
ACTCCATATGTTCCTTTTGTTCTAATCTTGTNAGCCAGNCCAANNGACCAAGGTCNAGTTCTTTATTTC
CAACACCCCTTGGNAAAACAGCGCNCCNCGNCCNAGAAAAGGATATTTGGTTGTTGCTATTACCCCCA
AATACAGTTCAAAAGCTTAATTGGACTATTTTTTTTACCAATTTCAATTTCCAANTGGCCCAATGGGGC
NATNAAAAAANAAACTTCACACTTTTTCCAATACCCTGGGGTTACNTTTNTTTGAATCCTAACNCCATT
TTAAAAGCAATGCCGGGGCNTTACCCTTTAAAAGAATTNCCTTTTTTTCTGGAACCANTTCACGCCCGG
AAAATTNGGAAAAAAAACTTTCCTGGTTTTAAAATAANCCTNAANCTTGGGNNANAAAAAAATTAAC
NGGAAATTGNTATGGGGNCCCCCCCAAAAAAATAAAAAAATTTTTTNCCCAAAAGN
NCANNATCTGGAGCTCCACCGCGGTGGCGGCCGCTCTAGAACTAGTGGATCCCCCGGGCTGCAGGCAG
AGCCCGCGGTCCGGACTGCAGCGCGTGCCCCAAGCTCTCCGTCTTCCGCCGCCCGCCAGCCCGAGCCC
AGCCCGCGGCCCCAGCAGCAGCCCCGAGAGCAGCCCCAAAAGCAGCGCCATGGCCGGGTGGAACGCC
TACATCGACAACCTCATGGCGGACGGGAGCTGTGAGGACGCAGCCATCGTGGGCTATAAGGACTCGCC
CTCCGTCTGGGCCGCCGTCCCCGGCAAGACCTTCGTCAACATCACGCCTGGTGAGGTCGGTATCCTGGT
TGGCAAAGACCGGTCAAGTTTTTTCGTGAACGGGCTAACACTTGGGGGCCAGAAATGTTCTGTGATCC
GGGACTCACTGCTGCAGGATGGAGAATTTACCATGGATCTTCGTACCAAGAGCACGGGTGGAGCCCCA
ACCTTCAATATCACGGTCACCATGACTGCCAAGACGCTAGTCCTGCTGATGGGCAAAGAAGGTGTCCA
CGGCGGTATGATCAACAAGAAATGTTATGAAATGGCTTCTCACCTGCGGCGTTCCCAGTACTGACCTC
GTCTGGCCCTCCCCTCACCACTCCCCACTGCTTCTGCACCCCTCTCCTTCCACACACATACATACACCAT
TTTTATTTTTTGGGGCATTACCCCACACCCCTTATGCTGGCAAAACCACATGGGCTGGGGGCCCCGGG
CTGGATGGACAGACACCCCCTGCCACAACCCATACCCCTTCTGTGTGTGGTTGGGAAAACTTTTTTGTT
TTTTTGGGTTTTTTTCTGAATAAAAAAAGAATTCTATTACAAAAAAAAAAAAAAAAAANCCTTGGGGG
GGGGGCCGGGGCCCCATTTNCCTTTTTAAGGGGGNGNNNNNNNNNNANNNNNNNNNNNNNNNNNNN
NNNNNNNNNNNNNNNNNNNNNNNNNNNNC
NAAAACTGGAGCTCCACCGCGGTGGCGGCCGCTCTAGAACTAGTGGATCCCCCGGGCTGCAGGANTTC
GGCACGAGGGGGGAGTGAAGATCTGCTCCTCCGGGTGCTAGCCATCACCAGCTACTCCATCCCAGAGA
GCATCCAGAGCCTGAAGTGCCGCCGCTTGCGTGGTGGTGGGGAACGGCATCGGCTGCGCAACAGCTCG
CTGGGAGAGGCCATCAACAAGTACGACGTGGTCATCAGGTTAAACAGTGCCCCTGTGGCTGGCTACAA
GCAGGACGTGGGCTCCAAGACCACCATGCGTCTCTTCTACCCTGAATCCGCCCACTTCAACCCCAAAG
TGGAGGACAACCCTGACACACTCCTCGTCCTGGTGGCTTTCAAGGCCATGGACTTCCACTGGATTGAG
AGCATCCTGAGTGATAAGAAGCGAGTGCGAAAGGGCTTCTGGAAACAGCCTCCCCTCATCTGGGACGT
CAACCCCAAACAGATCGGATTCTCAACCCCTTCTACATGGAGATCGCAGCCGACACAAACTGCTGAGCC
TGCCGATACACCAGCCACACAAGATTAAGCAGAAGCCCACCACAGGGCTGTTGGCCATCACCTTGCCC
TTCACCTCTGTGACCTGGTACACATTGNTTGGCTTCGGCTACCCAGACCCCACCANAAAAAGCAGTCC
ATTCACTACTATGAGTATATCACGCTCAAGTCCATGATGTGGTCANGCCCACAATGTCTCCAAGGAG
GCCCNTGGCCATTAAAGCGGGATGCTTGGAAAAATCGGAACCGGNCAAAAACCCTTAACTTTACTTTT
TGACTTNGGGCAANGGCTTGTTCCCATTGGGNNNAACCCTTGNTAAACNGGCCCCCGGCCCCNNCCTT
TTGGGGGGGGCCAGGGGCCCACATTGGGGCCGGTTTTGGACCTCACCTTTTTCTTGGGGGGGGGNGGG
GGNCCAGGNTTAAAAANAAAGGCCNCCCCCTTTGGTTTTTTTTGNGNGAAGNT
AAAANCTGGAGCTCCACCGCGGTGGCGGCCGCTCTAGAACTAGTGGATCCCCCGGGCTGCAGGCTCCC
GTGCTCCCTCTTTTGGCAAACTNGTNGAGTACATGACTCAGTCANACAGGAGNTCTACCTCACAGNCN
ACNTGTNNNCTANTNGCNAGANAGACTTGNGTCACACTAACGTGGNCCACNACCTGCCGGAAATGCA
TTGTTCANAACACAGAANGAAATTTANGATTTTTGCTTTTCTGTGACNGNATTGNTTCCGTCTCANNT
CNGAGGNGTNAACNGCATGTTNNTNTNGANANNGCNTATTTAACATTGATTGNGANAACCNGGGAAT
TNGGCTCAAAANCTNGGAGCNTGANAGGAACCTTATAATGANGCATAGNGTCATTCATTATANCGGG
ACCATNGANGGATNCTNTTTCATCCTCTCTCANNGNANAAATCANGACNNGGGTGGTTGTGGCANANG
GNTTTCCAACCTCGGTAGAANTAACTGNATTGCANGTTCNACTCCCTCACCGAGGNGACCTGNTTCGG
ATCTGTCTNAANGAGCANAAGACTNGATTGNTNNCANTGGCTCTAATAAGNAANAATNCNNCATTTAC
NACANGGTANNGGGGNNTTCNTTNTTTNNNCANTCTNNTCCNNGNCCATNCANCCACCCGANCCAATA
TTTCTNCTTCCCTAGNNNAAAAANTCTNTACCTTCNCTGTNATNTATATNTNNGNNGNGCNNNTTTTTN
TCTTACCTNTNTANNTNCCNCCGCAAGGANNCTNANAAGTTAANTNATTGNNTTNCATCNGCACTCGC
NNCTNNTNTAACGNNNTTGNTNTNTGGTCCCCNNANCNANTNGCCCNNATAATTNNCCCNTTCNTANN
TNTGNANCCANNTNNGCNNNCNNTTCTNNCTAGGATCGCCACCGATANNGNATATCCACCGNANTCTC
CCTCNCTTTNATANNGTNAGNAACACNNACNGNNTNNAAAGCNGCTCNTCNCNACNGNTTNTNCTAC
NANNNTTAACATANNNGGAAGNNC
NNCNATATTGTGTNATTATTTTTNTNTTNNNNTTNNNNNNANNCNNNNCCTNTCTNANANNNTCATCN
CAATNTTNCTTNATCTNGGNANAATTTANNNAGACCNCNATTTGAGNNACNCNGGANTNCATNACAGT
GGCGGCCGCTCTAGAAGGAGTGGATTCCCGGGNCTGCAGGAAATGTNCCNCACGAGGGGCNTNAANG
TTNCCNAGGTANGTNGGCTNCNNTGGGNGNNTTANTGTNNCANNAAGGACTNCACNCANACANTTTN
CATGATCAGTTCATTATNATTTCTTTNANNTTTTAAATCTNNNANTCCTCANCCACTCCCTCTTNATTAN
NNTANATCTTNNNNNNNNNNTNTTAATNTNNACCTTTTTCTGTATANNTNTCTCTGTNANCTGNGNCNC
NGTCTANNCNTTCNTANTCGAGCTCTNTAATTTGTNTACTTATTTCCNAAACTNNGTTTCTCATTCCCNC
NCCATANANNATNNTCNNTACATANNCGAGNCANATTTCCCNCTTTANTGNNTACCTTTTCANANATA
TATTGACNACGTCTTTTANNTTTTTNNNTNTCANTCATCANCNGATNNTNCANACTANTCTCCCNTNAN
ACCTANAACANTGTNTNAAANAACNCTATATTTACTCNTTATNTTATAATCNAGTGNTCNGNNNTACTC
GNATANTCANTATNGCNNANANGANCTAGNNGCCACNNTTNNGNNANTATTTTNGANTCTCCATNTTTN
TAGTCNCNCACACTANACTAACTTTNATNATTTCCTNTTGCAACAGNTNNGCCCTTNNTNNTAATNCTC
TACNTTNTTTATTCCACCNNANGACCCTCCNCTANTANTAACNNGATNANGCCNTTANTTNANNTACT
CCATCACNTCGTCTACTACANGAGGCNACNNCTNTGTTGANTNNNCTCNTCAANNNNTNNNNTNACCT
CTATTCTCNTNNTANTAGTTTCCCNNAATNTACTTNTCCGTATATNTNTNNTTAAGANCNNCTNNTAN
NATGAAATANNATTATTTNTATTANCTCTTCTNNTTANTTCTCTNCGGATNTTNTNNCTTGAGGNGACC
NCNTCATNTATNNCNTCTCNANAGNTNTTNTATNNNCTATNNNNNCANNNCCNTCTTACTAGTANACC
AATNNCTCAGNTCGGCNATNNTTATGTGCCCNCCNCNNNGTCGCCAGTACCTNAAAACCNTNGGNGAN
NCTNNGCTNNGNTTTNTGNAAATANAACCCCCCAATTNTTTATAAACNTGNNGNCNNNTCNCTTCCTCN
CNANCNNNTTNTACATACNCCCCTCTATCTTANATCNAANTANGTATTCANNNACNTCGCGTATATAT
AAACNTNNGNATNCTNTNCTCTATNATCTTATCNGTACNTCANACNNNACGTTNNTTNCNCCGATTTA
AGTNATTNNCNCGNNNNATCNNCCGCANCGTCTCTTTATNTATCTNNCNACTCATCTANNGTTACGTGA
CTNTCNATANNGTATCTAATTTTNNATTTNATTTTANG
NNCNATATTGTGTNATTATTTTTNTNTTNNNNTTNNNNNNANNCNNNNCCTNTCTNANANNTNTCATCN
CAATNTTNCTTNATCTNGGNANAATTTANNNAGACCNCNATTTGAGNNACNCNGGANTNCATNACAGT
GGCGGCCGCTCTAGAACGAGTGGATTCCCCGGNCTGCAGGAAATGTNCCNCACGAGGGGCNTNAANG
TTNCCNAGGTANGTNGGCTNCNNTGGGNGNNTTANTGTNNCANNAAGGACTNCACNCANACANTTTN
CATGATCAGTTCATTATNATTTCTTTNANNTTTTAAATCTNNNANTCCTCANCCACTCCCTCTTNATTAN
NNTANATCTTNNNNNNNNNNTNTTAATNTNNACCTTTTTCTCTATANNTNTCTCTGTNANCTGNGNCNC
NGTCTANNCNTTCNTANTCGAGCTCTNTAATTGTNTACTTATTTCCNAAACTNNGTTTCTCATTCCCNC
NCCATANANNATNNTCNNTACATANNCGAGNCANATTTCCCNCTTTANTGNNTACCTTTTCANANATA
TATTGACNACGTCTTTTANNTTTTTNNNTNTCANTCATCANCNGATNNTNCANACTANTCTCCCNTNAN
ACCTANAACANTGTNTNAAANAACNCTATTATTTACTCNTTATNTTATAATCNAGTGNTCNGNNNACTC
GNATANTCANTATNGCNNANANGANCTAGNNGCCACNNTTNNGNNANTATTTTNGANTCTGCATNTTN
TAGTCNCNCACACTANACTAACTTTNATNATTTCCTNTTGCAACAGNTNNGCCCTTNNTNNTAATNCTC
TACNTTNTTTATTCCACCNNANGACCCTCCNCTANTANTAACNNGATNANGCCNTTANTTTNANNTACT
CCATCACNTCGTCTACTACANGAGGCNACNNCTNTGTTGANTNNNCTCNTCAANNNNTNNNNTNACCT
CTATTCTCNTNNTANTACTTTCCCNNNAATNTACTTNTCCGTATATNTNTNNTTAAGANCNNCTNNTAN
NATGAAATANNATTATTTNTATTANCTCTTCTNNTTANTTCTCTNCGCATNTTNTNNCTTGAGGNGACC
NCNTCATNTATNNCNTCTCNANAGNTNTTNTATNNNCTATNNNNNCANNNCCNTCTTACTAGTANACC
AATNNCTCAGNTCGGCNATNNTTATGTGCCCNCCNCNNNGTCCCCACTACCTNAAAACCNTNGGNGAN
NCTNNGCTNNGNTTNTGNAAATANAACCCCCCAATTNTTTATAAACNTGNNGNCNNNTCNCTTCCTCN
CNANCNNNTTNTACATACNCCCCTCTATCTTANATCNAANTANGTATTCANNNACNTCGCGTATATAT
AAACNTNNGNATNCTNTNCTCTATNATCTTTATCNGTACNTCANACNNNACGTTNNTTNCNCCGATTTA
AGTNATTNNCNCGNNNNATCNNCCGCANCGTCTCTTATNTATCTNNCNACTCATGTANNGTTACGTGA
CTNTCNATANNGTATCTAATTTTNNATTTNATTTTANG
TNNANAAANNCTGGAGCTCCACCGCGGTGGCGGCCGCTCTAGAACTAGTGGATCCCCCGGGCTGCAG
GAATTCGGCACGAGGCCGGACACAAGGGGGAGCGTGGTTACCCCGGTAACGCAGGTCCTGTTGGTGC
TGCCGGTGCTGCTGGCCCTCAAGGCCCTGTGGGTCCCGTTGGTAAACACGGAAACCGTGGTGAACCGG
GTCCTGCCGGTGCTGTTGGTCCTGCTGGTGCTGTTGGCCCAAGAGGTCCCAGTGGCCCACAAGGTATTC
GAGGTGACAAGGGAGAGCCTGGTGATAAGGGTCCCAGAGGTCTTCCTGGCTTAAAGGGACACAATGG
GTTGCAAGGTCTCCCGGGTCTTGCTGGTCATCATGGCGATCAAGGTGCTCCCGGTGCTGTGGGTCCCGC
TGGTCCCAGGGGCCCTGCTGGTCCTTCTGGCCCCGCTGGCAAAGACGGTCGCATTGGACAGCCTGGTG
CAGTTGGACCTGCTGGCATTCGTGGCTCTCANGGTAGCCAAGGTCCTGCTGGCCCTCCTGGTCCCCCTG
GCCCTNCTGGACCTNCTGGCCCAAGTGGTGGTGGNTACAAATTTTGGTTTTTGATGGAAACTTNTACAG
GGCTGACCAANCTTGCTTACCAACTTTTTTTNAAACCCAAGGATTTATGAAGTTGATGCTTACTCTGAAA
TCTCTCAACAACCAAAATTGAAAACCCTTNTTACTTCAAAAAGGCTTTAAGAAAAAACCCAACTTGGA
CAATGCCCGAGACTTTGANACTCAANCCCNCCCAAAAATGGGAGCAAGGGGTTACTAACTGGGAATT
GGACCCCTTAACCCAAGGGATGGNCCTNTTGGGTTGCCTTNTTCAAAAGNATACCTGGGGAAATTTNT
TTTANTTGGGGGAAAAACTTGGNTTTCCNGGGCTTCAAACCTTGAAAAAAAATTCCCAAATNNAAAAA
AACTTGGGTACCAAAAAAAATTTCCAAGGGGCCA
AAAAGCTGGAGCTCCCCGCGGTGGCGGCCGCTCTAGAACTAGTGGATCCCGCGGGCTGCAGGNGAGG
GNAACCATGACATACAGTGATGCANGGAANAANACGAAGTGGANGGCANAATAGCTATAGGAAAGC
NGCTGGACTTGCACTGGAGGAGCTGGACAGGTCTTTTGGTCAGCTGGTCTTGCAGCCACACCTCTTGGT
CCTGGAGAGAGAGAAAANAANTGCTGNTAGCANAGCTGCTGAAACACCTCTTNCNTTCCCNANCCTCT
TTCACAAAGGCATTTGAACTCTTTGGCATTCTACACAATCTACACTTACGGATCCTCACTTGACACTCG
CCTTCCTCTGGCTCCCCTGTTTCAGGGCAGNANAAGCANANNGAGGCTTTTTGTGAAATGAAAGGCNG
CATGTNGGAAAAAGAATGNCTGGGAAATAGGACAGGAAACTCCTCTTNTAAAAAGATGGGTGTGGGG
TGACAANTGCAGGCACACCTGGNTTCAANTTCTANCTTCAATATATGACTGGCTATGGGGACTTTGGN
AANANNTTTAACCTTCTGGGCCTCATTTTTCCNTTGGAAAANGGANCAAACCCCTACCTTACANGGGT
GGTTGNGAGGATTAAATTAAATGANATGNGTATGTAAAGTTTCTAACNCAGNGCCTACATATTGGGGG
TCCTCAATAAAAGGGAACCAGCAGCTNAAAAAAAAAAAAAAAANNNNNNNNNNNNNAAAAAAAAA
AAAAAAANNNNNNNNNNTTTTTTNCCCNAAAAANNNTTTNNAAANNNTTNNNCCCCCCCNGGGGTT
TTTTTTTNAAANNNGNNNNNNAAAAANNTTTTTNNNNNNNNNNNNNNNNNNNNTTTNTNNNNNNNNG
GGGGGGNNCCCNNNCCCCCCCNCNNNNNNNNNNNNNNNTTTNNNNNGGGGGGNGNNNNCN
NAAAAGCTGGAGCTCCACCGCGGTGGCGGCCGCTCTAGAACTAGTGGATCCCCCGGGCTGCAGGCAG
CAGCCTAAATGTGCAGACTTTGTAACCAAGCCCACCCGAGATCGGTCTGTGCTTTTCACGTGACCACCA
TCTGTGCCTCCCTCGCTCCATCCAAATTTGTGTAGGCAGCTCCTTGGAGCCATTCCTAAAAATATAGCT
ACACCAGGCCCTGGAAACTGTAGTCAAGTAACAGGCTTAAGTGTTTTCCCTCCTTGAGTTAAACCTCCA
TTAGATCTCTTTTGAGGGACCTTTCAGCTGCAGGCATCAGTGTGTTCTGACAGCGAGACACGTTATGTG
TGTGTTTTCTATCTGGATCCAGGGCATCAGGATACAGTTCATCTTCTTGTACTTCCTCTCTGGCATGCAG
TATTTCTGGACTATGTTTATAAGTCTGTTTAGTAAAGGGTGGGATAGAGGATACCAAGGGAGTTTCCA
AGGGACAGAGATGGATCAGTTAACGATAGTCAGAAATGAACTTAAAAGTTGCCTTGAAAAACCAATC
ACATCTTATCAGGACGCATACCTTTATGTTTTCTAGCTCCTGTCGCTGAACTGGTTGCTGGGTAGCCCG
TACATTTCTATAAATCCTGGTGGACTCAGTTCTCACTGTACTTCCAAAGAACTTCGATCGNGGGTTTAC
ATGGACATGTATTTCAGGGGAGTGTTTACAAAGGGCGGATGGGCCCCATATANTTGCCTGGGNAAATG
AACCCCGGGGANGGGTCCTTTNAAATGGGCNAAGNTTTTTATTAACNCNAAAAAAAATAACCCCTTTT
GAAAAAAAATTGGGCNGGNNCCTTTGGGGGGGCGGGGGTTTTNTTTTTTNTGGCNNNNGGGGGGTNN
AGGGGNNANAAAAAACAAAGGGGTTTTTTTTATTTTTTTNGGGGGGGGGGTTTTTTTNNAGGNGGG
ACAANCTGGAGCTCCACCGCGGTGGCGGCCGCTCTAGAACTAGTGGATCCCCCGGGCTGCAGGCCGCC
ATGGGCTGTCTCGGAAACAGCAAGACCGAGGACCAGCGCAACTAGGAGAAGGCGCAGCGCGAGGCC
AACAAGAAGATCGAGAAGCAGCTGCAGAAGGACAAGCAGGTCTACCGGGCCACGCACCGTCTGCTGC
TGCTGGGTGCTGGAGAATCTGGTAAAAGCACCATTGTGAAGCAAATGAGGATCCTGCATGTTAATGGG
TTTAATGGAGAGGGCGGCGAAGAGGACCCGCAGGCTGCAAGCAGCAACAGCGATGGTGAGAAGGCC
ACCAAAGTGCAGGACATCAAAAACAACCTGAAAGAGGCCATTGAAACCATCGTGGCCGCCATGAGCA
ACCTGGTGCCCCCTGTGGAGCTGGCCAACCCAGAGAACCAGTTCAGAGTGGATTACATTCTGAGCGTG
ATGAACGTGCCGGACTTTGATTTCCCTCCCGAATTCTACGAGCATGCCAAGGCTCTCTGGGAGGATGA
AGGGGTGCGTGCCTGCTATGAGCGCTCCAACGAGTACCAGCTGATTGACTGCGCCCAGTACTTCCTGG
ACAAGATTGATGTCATCAAGCAGGATGACTACGTTGCCCAGCGACCANGATCTGTTCCGCTTGCCGNG
TCCTGGACTTCTGGAATCTTTGAAACCAAGTTNCAGGNGGACAAAAGTCAACTTTCCCATTGTTTGACG
TGGGCCGGCCAACCCCNATGAACCCCCGCAAATGGATNCCAATGCNTTCAATGAATGGGACTGGCCAT
TAATTTTTNGGGGGTGGCCANCANCAANTTTCNAAATTGGGGCATTTTNGGGAAGGAAAAACCAAAA
CCAAACCCGCCTGGGGNGGAAGGGCTTTTGNAACCTTTTTTNAANANN
GCCNATNTGGAGCTNCACCNCNGTGGCGGCCGCTCTAGAACTAGTGGATCCGCCGGGCTGCAGGCTCC
CATGCTCCCTCTTTTGGCAAACTGTAAGTACTGTCTGAGCAAACAGCGATAATTTCACAATTTCCTCTC
TAGAATTGCTGAGAGACTTGAGTCACCAAACGTGGTTCACTACTGTGGAAATGCATTGTTCAGAACAC
AGAAAGAAATTTAAGATTTCTGCTTTTCTGTGACAGTATTTGTTTCCGCCTCAGTTCAGAGTGTGAACT
GCAGGTTCCTGGCAAGACTGCTATTTAACATTGCATGTGAGAACCTGGTAAATAGGCAGAAAATCTTG
GAGCTTGAAAGGAACCTCAGAATGATTCATATGGTCATTCATTAGATTGGGACCTTTAAGGGATCTCTT
TCAGCCTCTCTCAGTGCAGAAATCAGGACAGGGGTGCTTGTGGCAAATGGCTTTCCAACCTGGGTAGA
GTAACTGCAATGCAGTTCACTCCTCACGAGGGAGCCGGTCCCATTGTTAAAGAGCAAAGACTGATTGC
TTTATTGGTTCTAGAAGCAAAGCCAACTTACAGAGGAAGGGGATTTCTTTAAACAGCAGTTCAAGCAA
AAGCCAGATTGAACAAATTTTGCTCCCTGAAAAAAATTTTACTTCAGGGGGAGTAATATTGTGGGNTA
TTATAATAATCANGAATCGTGAGAATGATGAAAATATAATGATGCTAGCCAGCTTAGTCCATTAGTAA
ANCACTGATTATGTACCACACACTGTCCTAAGTGCTTTATGTNNATTGAACCCNGTGTAAACTCTCAAA
AATAAACCCTTATGGAGANNGGGATACCATTATTCCTCACTTTTTTAAATGAAANAAACTGANGGCCC
AGAAAAGGNTTTTACAGTNNTTNCACANNTTAANTAACNNGTAGCAGGNGGGGNCNAGGNTCCAANA
ACCTNGGCNTCTTAACTACTGNGCTGNCCCTGCAGNGGGGANCCCNTGGNTNGTTNTNTCCCT
AAANCTGGAGCTCCACCGCGGTGGCGGCCGCTCTAGAACTAGTGGATCCCCCGGGCTGCAGGANTTCG
GCNCGAGGCTTGCTTCTCTAGGGAAGAATCCAACCGATGCGTACCTGGAAGCCATGATGAACGAGGCT
CCAGGGCCCATCAACTTTACCATGTTCCTCACCATGTTTGGGGAGAAGCTGANCGGCACAGACCCAGA
AGACGTCATCAGAAATGCTTTTGCCTGCTTTGATGAAAAAGCAACTGGCACCATTCAGGAGGATTACC
TGAGAGAACTGCTGACCACAATGGGAGACCGGTTTACAGACGAGGAAGTGGACGAGCTGTACAGAGA
AGCACCTATTGACAAAAAGGGGAATTTCAATTACATCGAGTTCACTCGAATCCTTAAGCATGGAGCGA
AAGACAAAGATGACTGAAGGAACTTGAGCTAAAACTTTCCAGTTACTTTGTCTTACTCTATACTGTTTC
CTAGACATTTCCCCCCACCCCCCATAGGCCTGTTGCATGCAACTTTGATTCACAGCTTTGCCTTTTTTTT
TTTTTTGGGGGNTTNNATTAAAAACNTCCNGNCCCTTAACCTNTTGTANAANCAANNNGNAAGGGGGA
AAAAATTTNAAATTTGTTTGAAAAAAAAAATTGGNAAAAAAAANNCCCNAANNGGGAACCCCCNGGA
ANTTTTAAAAAAAAAAAAAAANNNNGGGGGGGGGGGCCGGNCCCCANTTNCCCCTTTANNGGGGGN
NNNNNNNNAANNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNN
AAAAGCTGGAGCTCCACCGCGGTGGCGGCCGCTCTAGAACTAGTGGATCCCCCGGGCTGCAGGCCGCC
GCCCCGCCCGGTGTTACCGGTATTGTAAGAACAAGCCGTACCCAAAGTCTCGCTTCTGTCGAGGTGTC
CCTGATGCTAAGATTCGCATCTTTGACTTGGGGCGTAAGAAGGCCAAAGTGGATGAGTTCCCAGTTTT
GGGNCNCATGGGGGCAAGAAGAATATNAACNANTTTTCTTTNAAACCCTNGANGNTNGCCGNATTTG
GGCCNACNANNNCCTNGGGNAAANNTNGGGNNAAAANNGGTTTNANNTTCCAANGNCGGTTCNNCCC
TTTCNTTNNATTCCNATTAACAANANGNTGNCTTGGGCTNGANCTGATANANTNCAAANAGGNATNCN
CNGNGCCTTTGGAAAGCCCCANGGCACANTGGCCANGGTCCACATTGGCCANGNCATAATGGCCATTC
GCACCAAGCTGCAGAACAAGGAACATGTGATTGAAGCCCTTCGCCGGGCCAAGTTCAAGTTCCCTGGC
CGCCACAAANATACACATCTCTAAAAAANTGGGGATTTACCAAGTTCAACGCGGATGANATTTGAGA
ANATGGNGGGGAGAAAAGCGACTCNTACCNNACAGGTGTGGGGNGANANACANATCTATAATNGGG
GCCCNGNGGCAAAANGGGGGGNNCCTANACTCTTGAGNANATGAGGNNGTGCCCCTCCTTTTTTAATT
NCNCACGAAAAAANAAGCCGNTTTTTCTTGTGGAAAAAAAAAAAAAAAAAAAAAANTNCNNGGGGG
GGGGGGCNCCGGGNCCNAAATTTNCCCNTTTTTATNGNNNNGGGNNNNNNTNNTANNNNNNNNNNNN
NNNNNNNNNNNNNNNNC
GCCTATNTGGAGCTNCANCNCNGTGGCGGCCGCTCTAGAACTAGTGGATCCCCCGGCCTGNAGGGANT
TCGGCACAGGGGCAGAAGGTTCGCGAGGAGGTGGTTACCGGGGNCAATTCTGTTGACCAGGCCCTGA
TTNACCCNNNAAANAATTCNNGTTTCANCCCTNNCCCGGCTTCCNTTNTNNCNTTGGAAAAGNANGGG
AACCANTGGNTNNNTNTNGCTTTNAACNNTTNNGCCNATGCTTAAGNTTTNGCNNNGGNCNTTTNAAA
NNCCAATNATTTNAATGGNGCCNTNACNATGGANTGAACNNCCANAATGGAGAAAAATGGGANNNTN
NNGGAGAGAATGGNCCNAAGANNAACNNGCCTGTCTTGGAAATGGAAAANGAGAAANTCNAGTGTG
ATCCTCATGAGGCCCNNTGCTATGACAATGNGAAGACTTACNCCNNTGNGAGAANACTGNNACAAAA
GANTATNTNTNGGCCNNNTTGCTCCTGCNCATGCTCTGNAGGCNACNGGGGCTGTNGCTNTGACACCT
GTCGCAGACCTGNGGCTTGACCCCGGAACNAAAGGTCACTTGTNCACTGTACTACCAAGTATTTCCC
AGAGATGCNTCANAAAACCACACACTNTTGTNACCTGCCNCACNNAAGNCTTCNTTGCCCTTTGGAAG
TCCACGCTGNCACAAAAAATACCCNNGAAGTANTTATTTTCCCCCCCCAAAAACNAGCGNNGGTTTT
TTCCAAGGNCCTTCCNAAACTGGGGGGGNGGTTCNCAAAACCCCCCTGGTNNGGGGGGGNCTTTTNTT
TAAANNCCTTTTTTTCNTGGGAGGAAATTTCTTCNANCTTTNNNTTNAACTTNNNAANNTTTTTTCAAA
CATTNAACCCCTGGGGNGGGGTTTTAAAAATTTTTTTTCNTTCATNGGGGNGGNANCAGGTTTTNTTTT
TTTTTNCA
GCCTATNTGGAGCTNCANCNCNGTGGCGGCCGCTCTAGAACTAGTGGATCCCCCGGCCTGNAGGGANT
TCGGCACAGGGGCAGAAGGTTCGCGAGGAGGTGGTTACCGGGGNCAATTCTGTTGACCAGGCCCTGA
TTNACCCNNNAAANAATTCNNGTTTCANCCCTNNCCCGGCTTCCNTTNTNNCNTTGGAAAAGNANGGG
AACCANTGGNTNNNTNTNGCTTTNAACNNTTNNGCCNATGCTTAAGNTTTNGCNNNGGNCNTTTNAAA
NNCCAATNATTTNAATGGNGCCNTNACNATGGANTGAACNNCCANAATGGAGAAAAATGGGANNNTN
NNGGAGAGAATGGNCCNAAGANNAACNNGCCTGTCTTGGAAATGGAAAANGAGAAANTCNAGTGTG
ATCCTCATGAGGCCCNNTGCTATGACAATGNGAAGACTTACNCGNNTGNGAGAANACTGNNACAAAA
GANTATNTNTNGGCCNNNTTGCTCCTGCNCATGCTCTGNAGGCNACNGGGGCTGTNGCTNTGACACCT
GTCGCAGACCTGNGGCTTGACCCCGGAACNAAAGGTTCACTTGTNCACTCTACTACCAAGTATTTCCC
AGAGATCCNTCANAAAACCACACACTNTTGTNACCTGCCNCACNNAAGNCTTCNTTGCCCTTTGGAAG
TCCACGCTGNCACAAAAAAATACCCNNGAAGTANTTATTTTCCCCCCCCAAAAACNAGCGNNGGTTTT
TTCCAAGGNCCTTCCNAAACTGGGGGGGNGGTTCNCAAAACCCCCCTGGTNNGGGGGGGNCTTTTNTT
TAAANNCCTTTTTTTCNTGGGAGGAAATTTCTTCNANCTTTNNNTTNAACTTNNNAANNTTTTTTCAAA
CATTNAACCCCTGGGGNGGGGTTTTAAAAATTTTTTTTCNTTCATNGGGGNGGNANCAGGTTTTNTTTT
TTTTTNCA
TTNNNAAAAGCTGGAGCTCCACCGCGGTGGCGGCCGCTCTAGAACTAGTGGATCCCCCGGGCTGCAGG
GCTGCGAGTACCTCATGGAGCTGATGACGCCAGCTGCCTGCCCAGAGCCCCCGCCTGAATACCCAGTG
GAGGGCGACCATGATGAGCTCTAGCCCTGCCCTGCCCTGCGCAGTGCAGAGAAATCAAGATGTGGAAC
CAGCCCCCAGTGGTGCTCCCCATCCGCCCCTGAGTCTGTGGACCAAGGCCTGCCGNTATGGGTCACCT
CACCCACCCCACAGGGCAGGAAGCAGGTAGGGCTCCATGCCCTGCTTTTGGTTGGCCAGTGGGGCAGA
GCCATGTTCCCAGGCCCCTGCTGCCTCCACAGATGGAATAAAGGAGCCTGTCCTGCTTGGGCCCCCAC
ACCCTGGGGGCCTGGCCCGCTGTGCGCCCCACCCAGGGTATCTGCCTTCCCTCTAGGTGGGGAAGTGA
CTTGACCTGTGACCCCCAAACAATAAACATGATCCCTCCAAAAAAAAAAAAAAAAAAACNTGNGGGG
GGGCCCGGGNCCCNATTTCGCCTTATGGGGNGCTNNTANAAANNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNANNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNN
TNNANAAAACTGGACTCCACCGCGGTGGCGGCCGCTCTAGAACTAGTGGATCCCCCGGGCTGCAGGG
GCGGCCGGGGCCACTTCCTGTGCTTCGTCTACCCGCTGGATTTCGCCCGAACCCGCCTGGCGGCCGAC
GTGGGCAAGTCGGGCAGTGAGCGCGAGTTCAGGGGCCTGGGAGATTGTCTGGTGAAGATCACCAAGT
GGGAGGGCATCCGCGGGCTGTACCAGGGCTTCAACATGTCGGTGCAGGGCATCATCATCTACCGCGCC
GNCTACTTCAGGCATCTACTACACGGACNAGGGCATGCTCCCNGACCCCAATAACACTCACATAGTGG
TGAGCTGGATGATCGCGCAGACCGTGACGGCCGTGGCGGGCGTGGTCTCCTACCCCTTCGACACCGTG
CGGCGGCGCATGATGATGCAGTCGGGGCGCAAAGGAGCCGACATCATGTACAAGGGCACCGTGGACT
GCTGGCGGAAGATCTTCAAGGACGAGGGCGGCAAGGCCTTNTTCAAGGGCGCCTGGTCCAACGTTCTG
CGCGGCATGGGCCGGCGCCTTCGTGCTGGTCCTNTACNACNAGCTCAANAAGGTCATNTAGGCGCCCC
GTCCGCCGGCCGGGAACGGCTAACGCCATNCCTTATTGGGACGGACCACCGACTTCNAAAAATTCAAC
GTCCCCGCCGGGGCCGGGCCACCCCGGCCGNGGGGNTTNTTTGNNGGGGGACCATTGGNCCTGGGTCC
NCCCCTTTAATTGGGGGGGGGGGNAAAGGCAATCGGGGGGGGTNTGCTTGGGTNCCCAAGTNNAGGG
AANTCCCNNAACCCAAACTTAAAATTCCGGGGGCTTTTGAGGNACCCAACCCGGGNTTTAAATTTTT
TTTTTNAAAAAAAACCTTGGNTCCATTTGGACTTTAAAACCCTNTTCCTTTTTTTTTTGCCAT
TTNNNNANANNCTGGAGCTCCACCGCGGTGGCGGCCGCTCTAGAACTAGTGGATCCCCCGGGCTGCAG
GAATTCGGCACGAGGCTGCTGCCTGCCTGCCTGCCACTGAGGGTTCCCAGCACCATGAGGGCGTGGAT
CTTCTTTCTCCTTTGCCTGGCCGGGAGGGCCTTGGCAGCCCCTCAACAGGAAGCCTTGCCTGATGAGAC
AGAAGTGGTGGAAGAAACCGTGGCCGAGGTGGCCGAGGTACCCGTGGGAGCCAACCCCGTCCAGGTG
GAAGTAGGAGAATTCGATGATGGTGCCGAGGAAACCGAGGAGGAGGTGGTGGCCGAGAACCCCTGCC
AGAACCACCACTGCAAACACGGCAAGGTGTGTGAACTGGACGAGAACAACACCCCCATGTGTGTGTG
CCAGGACCCCACCAGCTGCGCTGGCCCCATCGGCGAGTTTGAGAAGGTGTGCAGCAACGACAACAAG
ACCTTCGACTCTTCCTGCGACTTCTTTGCCACCAAGTGTACACTGGAGGGCACCAAGAAGGGCCACAA
ACTCCACCTGGACTACATCGGGCCTTGCAAATACATCCCCCCCTGCCTGGACTNCGAGCTGACTGAATT
CCCCTTGCGCATGCNGGACTGGCTTAAAAACGTCCTGGTCACGCTGTACGAGANGGACGAGGACAAC
AACCTTCTGACCGANAAACAANAAGCTTNGAGTGAAAAAAAAATCCACGAAAAATGAAAAACCGCCTG
GANGGTTGGCGAACCATTCTTGNGGAAATTGGTGGGCCCCGGANCTTTNAAAAAAAAATTACCAACAT
GGTCGATTTTTTCCTTGGGCCCCTNGGAAGTTNGGGGCCANTTTGGATCAANCNCCCCCCNTTTGNNGG
GGTNCCTTTTTTTAAAACCCGGGGNTGGGCCCCCAATTNGGGCNCCCCCNTTTTTTTCCCCANGGGAAA
CCCTGGCNCCCCCCCCCNTTTTT
TNNNGNNANCTGGAGCTCCCCGCGGTGGCGGCCGCTCTAGAACTAGTGGATCCCCCGGGCTGCAGGC
AGCAGCCTAAATGTGCAGACTTTGTAACCAAGCCCACCCGAGATCGGTCTGTGCTTTCACGTGACCAC
CATCTGTGCCTCCCTCGCTCCATCCAAATTTGTGTAGGCAGCTCCTTGGAGCCATTCCTAAAAATATAG
CTACACCAGGCCCTGGAAACTGTAGTCAAGTAACAGGCTTAAGTGTTTTCCCTCCTTGAGTTAAACCTC
CATTAGATCTCTTTTGAGGGACCTTTCAGCTGCAGGCATCAGTGTGTTCTGACAGCGAGACACGTTATG
TGTGTGTTTTCTATCTGGATCCAGGGCATCAGGATACAGTTCATCTTCTTGTACTTCCTCTCTGGCATGC
AGTATTTCTGGACTATGTTTATAAGTCTGTTTAGTAAAGGGTGGGATAGAGGATACCAAGGGAGTTTC
CAAGGGACAGAGATGGATCAGTTAACGATAGTCAGAAATGAACTTAAAAGTTGCCTTGAAAAACCAA
TCACATCTTATCAGGACGCATACCTTTATGTTTTCTAGCTCCTGTCGCTGAACTGTTGCTGGGTAGCCC
GTACATTTCTATAAATCCTGCTGGACTCAATTCTTACTGNACTTCCAAANAACTCGATCGGGGGGTTTA
CATGGTNCATGTATTCANGGGAAGNGTACCNAAANGGNNGGATGGGCCCCATTATTTTGGCCTGGGN
AAAAGAAACCCNGGGAAGGGGTTCNTTTNAAATNGGGCAAAAATTTTTTTTTAATCCNAAANAAAAT
AAACCCCCTTTTGNAAAAAATTNGGGCNTGGGNCCTTTGGGGGGGCNGGGGGTTTTTTTTNTTTTTGGC
CNTTNGNGGGGTAAAAGGGGANTAAAAAAAACCAAAGGGGTTTTTTTTACAT
GNCCCTTTTANGTGNGGTCNNANTNANGNGGGGNATNTTCTCTTTTTNANCNTCTCGTNTTTCCTCG
GNTCTCGCNTCNTCNACTCCCNCATCNATATGTNNACTTGTTAACCCNNNTGACGCNCGNGGCGTGNT
NCCTGNGGGGGCNNNTATAGNGGTGNTCTTCTATTGNTNANNTNTNTNCGAANATCTTNTGGGNTTNN
GGACCANTNAAAGANNTNNTNCTNTAAGCNCCNANTNCCNGNTCNTNTNNTAGNNAANANNACNNNTA
TCNTGCTNGCNATGCTCGNAACNNCTNNNNNTTTNNATGNNGTNNTAANTNTNTNNCNGNNNNNGNAGT
TGNANATNTTNGCAATCNGCNNAAAGTGNTGAGCGGNATNGGGATTGTTGNNNNNACCTTNTTNANN
ANNTCNCNTNTCNGANAGNTNGNCAGTANNNTGGANTNGNGGNNNNNCANTNNGCACNGTTGGCNA
GNGNTGGGNAGTNNTNTGTNANNANTNNTTNTNTTCGCGNCAANNNCNNCGGNTTCNCNAAGTAAAN
ACTNTCTCAGNCNCNCAGANTGNNGNTNATGNTTNNGNNNNANCAGTNCAGNCTGNANGCNTANGNT
AAATANNGACGNAGTCGTAATCACNGGGNCTACNGNGNGGANCCNNANNANNGTNTNNGGNTNNTCT
NNCNGNGTTNCNGAAGCCNNNNGNTNNANNNTNNNCTAATGTGGAGGGAGTATGNNNTGGNTAATNT
TAGNANGCCTTGTNGNTNGNCCNAGNTCTATTNNNNNTNGGNGGTGGGNANNCTANCANNCNNNGGA
CNGANTNNAAGGAANAGNGGNNGNGGTNTNNTGNGNNNNNNNNTTTGGAGGGGCCGGGTGGGGGCG
NTAGNNGCNGNANAATNTTNGCNGGGNTNGNTAGTNNAANGNNCNNNTCNCCTGCNACAGNNGNTN
GGNNGGANGNGANNCNAANGACNNATNNAGCNAGNCCNNGNNAANTNGGCGCAGNANNTNNNCTNN
NAGGCNNGGNNGTGNGNTGTAGTNCTGNNNCNNNCCAGAGNACNGANNTGCAGCGNANGTGNTNAG
GANNNCGGAANCNGNNNAATNAGNACCGGTNCANNTAAGCANTNCNTNNNANTNNAGACGTGCNTT
GATAGNNCNNTGCNGCNCAGNGCNGCGANNTAATATNNGANCATAGCGGANCCTCGTATCTNTAGNN
AGCTGNANGANTACCTCGNNNCNGAGNTCNANCNTGTGANNNANGGAGAGACANAGCGNGGNNNAG
ANATGNNNNNTNGTNTGTNCGACTGAGNTGNNTNATNTANAGAGGTGCGNCG
GNCCCTTTTANGTGNGGTCNNANTNANGNGGGGNATNTTCTCTTTTTNANCNTCTCGTNTTTCNCCTCG
GNTCTCGCNTCNTCNACTCCCNCATCNATATGTNNACTTGTTAACCCNNNTGACGCNCGNGGCGTGNT
NCCTGNGGGGGCNNNTATAGNGGTGNTCTTCTATTGNTNANNTNTNTNCGAANATCTTNTGGGNTTNN
GGACCANTNAAAGANNTNNTNCTNTAAGCNCCNANTNCCNGNTCNTNNNTAGNNAANANNACNNNTA
TCNTGCTNGCNATGCTCGNAACNNCTNNNNNTTTNNATGNNGTNNTAANTNTNNCNGNNNNNGNAGT
TGNANATNTTNGCAATCNGCNNAAAGTGNTGAGCGGNATNGGGATTGTTGNNNNNACCTTNTTNANN
ANNTCNCNTNTCNGANAGNTNGNCAGTANNNTGGANTNGNGGNNNNNCANTNNGCACNGTTGGCNA
GNGNTGGGNAGTNNTNTGTNANNANTNNTTNTNTTCGCGNCAANNNCNNCGGNTTCNCNAAGTAAAN
ACTNTCTCAGNCNCNCAGANTGNNGNTNATGNTTNNGNNNNANCAGTNCAGNCTGNANGCNTANGNT
AAATANNGACGNAGTCGTAATCACNGGGNCTACNGNGNGGANCCNNANNANNGTNTNNGGNTNNTCT
NNCNGNGTTNCNGAAGCCNNNNGNTNNANNNTNNNCTAATGTGGAGGGAGTATGNNNTGGNTAATNT
TAGNANGCCTTGTNGNTNGNCCNAGNTCTATTNNNNNTNGGNGGTGGGNANNCTANCANNCNNNGGA
CNGANTNNAAGGAANAGNGGNNGNGGTNTNNTGNGNNNNNNNNTTTGGAGGGGCCGGGTGGGGGCG
NTAGNNNGCNGNANAATNTTNGCNGGGNTNGNTAGNNAANGNNCNNNTCNCCTGCNACAGNNGNTN
GGNNGGANGNGANNCNAANGACNNATNNAGCNAGNCCNNGNNAANTNGGCGCAGNANNTNNNCTNN
NAGGCNNGGNNGTGNGNTGTAGTNCTGNNNCNNNCCAGAGNACNGANNTGCAGGGNANGTGNTNAG
GANNNCGGAANCNGNNNAATNAGNACCGGTNCANNTAAGCANTNCNTNNNANTNNAGACGTGCNTT
GATAGNNCNNTGCNGCNCAGNGCNGCGANNTAATATNNGANCATAGCGGANCCTCGTATCTNTAGNN
AGCTGNANGANTACCTCGNNNCNGAGNTCNANCNTGTGANNNANGGAGAGACANAGCGNGGNNNAG
ANATGNNNNNTNGTNTGTNCGACTGAGNTGNNTNATNTANAGAGGTGCGNCG
TTGAGNCCNANTAGGGGGGNCGCATNNCNGGGGGGCGGCTCGCTCTTAGACCTAGTGGGATTCCCCC
GGNNCTNGCAGGGANTTTNTGGTNAATNTGANNGAGNTNTTCATNCATNTTTANCNCNATTCCCNAAT
ATNANAAANCANNGGGGGCCATNCCNNTNTNANNANNTGGGNTCNTGANAACCTAATTNNTNGCGAN
NNTCTNATCTTTANNNNNAGGNNNNTGANTNGGATGNAGGNANNANCANATNANNTAGTCANAGAA
NCTNTATGATTCANCGATATNNATTNACGTTACTGCGCGNNTANCNNTGNNNNNNTNANATNAGACN
AGAANANNGANATNGACANGNNATNNCTNCTGTCNNAATCNTTCATANNNNNNNCATGANTANCTTA
GTANACGNTGCTNNNNNTNNGCNNGTGTNNTNNCTCCCTTTCTCNNAGCATGNGNNGTCNNAGAGTNT
ANNNAGNGGTNNNTNNCNGTGANTAATCTGATNNTNACCCNGTGANNNGNGACGACNCNNGACAGN
GNTNNCTAGNGTGNTANNCGANNNGNNNTNGNNCNCTNATNNNCNNCCGNGTANAAGATGTANNCTT
TCTNCATNGANCTGNNNNNCGANGNNNNNTGCNTAAGTNGNTTCAACTTATNTGNGNATNNTNTNAN
NCTNCNGTAGTCCCNATGATAATATNTNGTATNNTGTGTNACTCTGGANCNCNNNCNGTGGATNTANT
TATCNATNAATTNTCCGAANGNNNNTGNGGATNNNNTNCANTNNCGTTNACNCNGTNTGATNTNANC
NTCGANAGANTNTNCTNTATCNCNNNNTANAGANNCTNATNNAACNNGAANNANTNGNTGNNGATTN
ANCNATNAGTCNTCTCTGAGNANGATNNNTTNNNTCNNNAANATNACNATATNGNNTNNGCGNGCTN
CGNANNTNNNATTTGGGTCNTTTNNNANNANCGNNNCTCNNTTNCTCGNGGNGACGNNGNGGCGGGN
NNCGAGTANNANGNCNNANNCNNNTGTCCNNTGGCNNNAGANNNGNCNNTNTGGCNTNNNAANANN
TGAAGTCTACCNTNNNNNNNATCGANNTCGATGTGTANTNNAAGTNNTGTATTNNTCANCANCAGNN
CACNTGGTNANNGANNTAGANGATGNTCTTCCATNNGATAANCATTTNGNGACGNACCNNGAGAGTA
TGNNTAANNATCNNANNCTGTNANGANGACGNANGTACNNTNNGTGGTATGAGGNCNGAGGACNGA
NGGAANAAAGNTAGAANCNATGGAGGTTCGACGTNCGNACNNACGACGNNANNTCNTACNGNTAGG
TANTNTCGNCNGAANGNNTCTCCNGTCNCNNGNATCNTCGTNANGTNTGNACGNCGNGAAGCGANGN
GATNNATNACNNGNANNAACGNAACTTAANNNNCGAACGTACGAANGGGACGTACGGNCNCACGCG
AATGAANGTAGGTGACTCACGNCNCGCGNTNNTANNNGANNTNTNGNATAGGNCGCNCTGNNNNNTA
CNCNACGTCGNACGGGAANGANAGNAATGNCNGAACGNTTACGCG
NAAAAGCTGGAGCTCCACCGCGNGGGCGGCCGCTCTAGAACTAGTGGATCCCCCGGGCTGCAGGAAT
TCGGCACGAGGACCAATGACAAAGATAGGAATACAAAGTGTATTAGTTAGCCATCATTCTTGCACTAA
GCACCAGAAGACTTTGAAAAATCTAAAAAACAAAAAAAAAATCTCATTNAACTAGGATAATTGAAAAG
GAATATTAAATCGATAAAACCAGGACTCACATGCCAAAGTTTGTCATCAGNGGACGCTCACATNTTGGT
TCCTTCCATATAAAACTTGGTTGCCGNTNTATTTAAAGNCTTTTTTTCAAANATTTTCATTGNGTTTCTG
NTAAANCTAAANCTNTGNTCANNGCCNNTACNAAACANGGGACAATTCTTANACTGGCTTGAAAATC
CCCCCGGNANAANCATGNGCNACTNTGNAANANTTTTTGNTCAAAAACATTACTGNCTGAAGCAACNT
TCCTTTCTATTTCTTANTGNTACNAAGGACCNGCAGGGANAAATGNCCTNTCCCCANCAGNGAATACT
GGTATGCAATTTACACTTGATGCTCAGGCCTGCTGAAGCTGAGGCTTACCTGTAATCATANCCTTCCAG
GGGATGAANTCTTCAGTTCGTCTAATAAGTNCCCTAACGAGTGTGAAAGGCACTTTGAGGGGATTTTA
TTAACAAAAACTTGGTTCTTTTATAAAAACNATATATTCATTTTCCCNCATACTATAAAGCCNNCTGNG
ACCTACCTACCTCTCAAAANGGACCTTTTTGGGGAAAAAAGCTNACATTTTTTAGGGGAAAAAAGGAT
CCCCTTTTTACCCGGGGGGTTAAGATTTTTTTGGNATTGGGGGNCCATAANANCANGTTTTTTTTCAAT
GNTATTTTACCAGGGAAAATCNTTTGGAATNANAACCTTTTACCCCCCGACCCAAAA
GCCTATCTGGAGCTNCANCNCNGTGGCGGCCGCTCTAGAACTAGTGGATCCCCCGGGGTGCAGGCTGA
GGAACCCCGGTGGCAGGGCAAGGTTCGGGATCATGAGTTGGATTCCTTTCAAGATTGGNCAGCCCAAN
AANCAGATTGTCCCCAAANCAGTGGANAGAGANTTTGANCGANAGTTTTGGAAAANTCCANCAGTTGG
AGNACCAAACCAAAAGGNTGCAAAAGGNCTTNAAAAAAANCNCGGNCCCCNNCCTGGCCNTGTTTAA
NTNNGNTGNNAAAATNTCNTNGNNNTNGNTNTCCANCCCCNTTTGCAANNAAAACCAGNANTTTTTNA
NCNTGGNNANNGCCTTTGNNCNCAGCCNTNAANNGGNNGGNTNCNTTCACCCAGGAAAAGGTGACCCA
AATCCANAGAANTGTNATNGANCCTTNAAAAAAGTTCGGCATGTGTGTTCCCNANTCTAAATTGGCAT
GANAACCGGNGGAGCCAGGCNTTGCAGNANTACAAAAGGGTGTGNACNCCAGGGTGGAAANNNCCA
AGGANAGGGAAAACACGGCCCNGGCTCTGGCCAANNTCCACCAGGCCCGAAAAGATTTACGCCATTT
CGGATANGNNTTTGGGGCCAAAAAACACGCATTNTTGNGTNNGANANCCGCNNTTATACANACCGAG
TGTGGTATTTTTNCACCCCATTTTTGGTNCNTTTTANNANAACCAAGNGGTTGGTGCTCTNCTTNAAAN
TCTGCACAATATTTTTNAGAATTTTTNNCNCCAGCNTTTTTCACCCACCCNCTTTNTCTCTTTGAANAAC
CTNNGGGGAGGCGNNAAAAAACGGAGGCCCAGTTTTNNAANANATCTTCNNAACCNCNTTTTTCNTTG
NTGGGNANNAANAANTTNCCCCTCCCGGTNGGNAAAANTCTTGTTGANAANAAAACNACCCNNCCTT
CCTTTNTGCCCCTCTTTTTAAAA
GCCTATCTGGAGCTNCANCNCNGTGGCGGCCGCTCTAGAACTAGTGGATCCCCCGGGCTGCAGGCTGA
GGAACCCCGGTGGCAGGGCAAGGTTCGGGATCATGAGTTGGATTCCTTTCAAGATTGGNCAGCCCAAN
AANCAGATTGTCCCCAAANCAGTGGANAGAGANTTTGANCGANAGTTTGGAAAANTCCANCAGTTGG
AGNACCAAACCAAAAGGNTGCAAAAGGNCTTNAAAAAAANCNCGGNCCCCNNCCTGGCCNTGTTTAA
NTNNGNTGNNAAAATNTCNTNGNNNTNGNTNTCCANCCCCNTTTGCAANNAAAACCAGNANTTTTTNA
NCNTGGNNANNGCCTTGNNCNCAGCCNTNAANNGGNNGGNTNCNTTCACCCAGGAAAAGGTGACCCA
AATCCANAGAANTGTNATNGANCCTTNAAAAAAGTTCGGCATGTGTGTTCCCNANTCTAAATTGGCAT
GANAACCGGNGGAGCCAGGCNTTGCAGNANTACAAAAGGGTGTGNACNCCAGGGTGGAAANNNCCA
AGGANAGGGAAAACACGGCCCNGGCTCTGGCCAANNTCCACCAGGCCCGAAAAGATTTACGCCATTT
CGGATANGNNTTTGGGGCCAAAAAACACGCATTNTTGNGTNNGANANCCGCNNTTATACANACCGAG
TGTGGTATTTTTNCACCCCATTTTTGGTNCNTTTTANNANAACCAAGNGGTTGGTGCTCTNCTTNAAAN
TCTGCACAATATTTTTNAGAATTTTTNNCNCCAGCNTTTTTCACCCACCCNCTTTNTCTCTTTGAANAAC
CTNNGGGGAGGCGNNAAAAAACGGAGGCCCAGTTTTNNAANANATCTTTCNNAACCNCNTTTTTCNTTG
NTGGGNANNAANAANTTNCCCCTCCCGGTNGGNAAAANTCTTGTTGANAANAAAACNACCCNNCCTT
GCTTTNTGCCCCTCTTTTTAAAA
TTNNNNAAAAGCTGGAGCTCCACCGCGGGTGGCGGCCGCTCTAGAACTAGTGGATCCCCCGGGCTGCA
GGTTCCTGTTAACTCTTAATAAACTTACACATGCAAGCATCTACACCCCAGNGAGAATGCCCTCTAGGT
TATTAAAACTAAGAGGAGCTGGCATCAAGCACACACCCTGTAGCTCACGACGCCTTGCTTTAACCACAC
CCCCACGGGAAACAGCAGTGACAAAAATTAAGCCATAAACGAAAGTTTGACTAAGTTATATTAATTAG
GGTTGGTAAATCTCGTGCCAGCCACCGCGGTCATACGATTAACCCAAGCTAACAGGAGTACGGCGTAA
AACGTGTTAAAGCACCATACCAAATAGGGTTAAATTCTAACTAAGCTGTAAAAAGCCATGATTAAAAT
AAAANTAANTGACGAANGTGACCCTACAATAGCCGACGCACTATAGCTAANACCCAAACTGGGATTA
GATACCCCACTATGCTTAGCCCTAAACACAGATATTTACNTAACCAAANTTNTTCGCCAGAGTACTACT
AGCACCAGCTTAAAACTCAANGGACTTGGNGGGGCTTTATATTCCTTTNTAAAGGAGCCTGTTNTATA
ATCGATAAACCCCCGATAACCCTCCCCAATTTTTGGTTAAAACAGGTATATATCCCGGCTNTTTTTAGG
AAACCCTTAAAAAAAAAAAAAAAAAACCTGGGGGGGGGGGCCCGGGGNCCCAAATTTCCCCNTTAAG
GGGGGGNNNNANNAANNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNN
TNNANCAAANCTGGAGCTCCACCGCGGTGGCGGCCGCTCTAGAACTAGTGGATCCCCCGGGCTGCAG
GCCGTGACAAAATGTGCTCCTGGTCAAAAAGTGCCCGACCATGATCGANNCTCNCCTTCATGTCAAGA
CTACCGATGGTTAGTTGCTTCGTCTGTTCTGTGTGGGTTTTACTAAAAAGCGCAACAATCANATTCGGA
AGACCTCTTACGCCCAGCACCAGCAGGTGCGCCAGATCCGCAAGAAGATGATGGAAATCATGACCCG
AGAGGTGCAGACAAATGACTTGAAAGAGGTGGTCAATAAATTTGATTCCAGATAGCATTGGAAAAGAC
ATAGAAAAGGCTTGCCAATCTATTTATCCACTCCATGATGTCTTCGTTAGAAAAGTAAAAATGCTGAA
GAAGCCCAAATTTGAATTGGGAAAACTCATGGAGCTCATGGTGAAGGTAGTAGTTCTGGAAAAGCTCT
GGGGATGAGACAGGGTGCTAAAGTTGAACGAGCTGATGGATACCAGCCACCATCCAAGAATCGGTTT
AAAATGCANACTCTTAATGGGGACAAATAAAANATCTTATTTGNGGTNNNNNNANNNNNNNNNNNNN
NNNNNNNNNNNNNNNNNNNNNNNNNTNNNNNNNTNNNNNNNNNNNNNNNNNTNNGGGGGGGGGGGGG
GGCCCCCCCCNTTTTTTTTTTGGGGGGGNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNN
TNNAGCCCTATCNGGAGCTNCACCNCNGTGGCGGCCGCTCTAGAACTAGTGGATCCCCCGGGCTGCAG
GAATTCNNNGCNAGGNTNAGNNNTANGNTGNTGGGGGGGNTNNNTGNGTGGNGGACNGGGGNGGGN
ATANNANNNNCNNTTNTNGANCCNNGNGCTTGNCTGCATCTNCNGCNCNGGAGATGAACNNTATTTA
CAATGNCTTAGATTNNNNNNNTGTNNTTCTANTTTCNNNNNGCCTGNANCNGNANANGTNNGANGCN
TNNNNCAATGTGNAGCAGGACCATGATACNACAGGCTTGGANATGGAAGGTTTGTNAANTCNNTNTT
ANCCATGTGAGCNGGCNTTGGACNNCNTAGNATNAANAAGTCGNATGNAGTAACCNNGNCGNAATGC
TGNGGGACCTGAACCNNTCATACTGACTGTGCTTANNAGNNGNGNTCNATGATNCNGGCNTCNANNC
CCCCCCCNAGCATGGNNNCNCCCTANANNNANGTACCANGCTTGNNANATCTNNNCATANCGCCGAC
CGGANCNTATTTCAGTCCNNAACAGATGGATNCATNNNNTAANCGCTGTNGGCCGGNNTGNCTGANA
GTCCNAACNCNAACCTTNGAACNTNGAACTTNCTGGCAGGNGAATAANNTTACCTNGTCCCATNTTGA
NNANGGAATGATCAGATTTAAANACCTGNAAGTGGGNGAAAAAAATGACTANTAANGGGGACCCCCCC
NCCTGAATCNCGAATGAATTTGTGCACTTGGGCGGGAAGAAACTCANCATGGNTTCCTNAATCNTAGN
CCNCCNCCGCTTTGAAAGAAAAACTTGGNATTGGGGNAAGGGACTTCANNCTTTTCNGGNNNAAAAAT
NTTCCTTNATGNAACCTTGNNGGTATTTGTTTANGAAAAANAAAAAAAAAATTTNNAANGGGGGGNCC
NGAANTTGNANTTT
TTNAGNGCTTNTNNNGGGGNNGNTTNGCAGGNGGCGGCCGCTCTAGAACTAGTGGATCCCCCGGGCT
GCAGGCAGNATCCTANATGTGCTNACTTTGAAACCAAGCCCANCCGAGATCNNTCTGGACTGGATTNG
ANTGAAAACTGGCNATAAAATCCGGTACAACTCACGGGAGACGACATTGGAGAAGAACCAAGCTGGG
TCTATANGAAGCAGGCTGGGTCTATGAGAAGTGGNCTTAACATGCAAACCACTTTTTTAAGNAGCCNN
ATTACAATGAANACATCACTACTGTAATGCTTGNNCCATGATGTNANTTGCTNANTATCAGACTGAGA
CCCANCNATAAATATANANCGTTGNAAAAANAAAAAAAAANACCTGGGGGGGGGANNATGCCCTNA
TTTACCNCTGGCATGCAGTATTTCTGGACTCTGTCTCTAAGGTGGTCCTGNAGAACANGGATNTCCACG
TTGACTTGTNCTTGNNGCCGGGNTGNCGNCCTGCANGTAGGCCTTTNGNATCTNGNTNACCTTNGTGA
CTAAGAGANCAAGAATTNTCATTTNGCGTTATTNGGACCCTTTNTAANCTATTTAAAGCTNNTTGGGAT
AANNAATCCTCAATTTTTTNAAANTTGGNCCAGNTNNTNTNCAATGGTCAGGGAATCNTTTCNNTNGT
NGCTGGAANNACTTTCTTTTNAATGGGNGNTAANAAAGTNNANGCNANTGGNNTCNNTTTTCANANTT
NANGTNNANANNCCTNGTCTATCTTNTNTTTTCCCCGTCCCTGGTNCNGTCTANCCCCCNNNTGCCTTC
CTNNATGTCTGGNAAANACCTAGANTTTCNCTTTTTTTATAAATCTTACTNGCCTTTTTTTTGGNGGTTN
CCNTTCGTANTNAANCTCTTGTTCTGGGNNCTNTCTNNACCCNANNNCACACACNNAATTCTNGTGCC
TTTTTTTNTCTGTGNATNNTNNGTANTTTTCNTCCATNTTNCCNAGATATCTNNNAGNCTTANNTANGT
GTTTCC
GCCTATNNGGAGNNGCATNACAGTGGCGGCCGCTCTAGAACTAGTGGATCCCCCGGGCTGCAGGAAT
TCGGCACGAGGGCTGACAATGACATCGGAGCTGTCTCAACGACAGGGCACGGTGAGAGCATCCTGAA
GGTGAATCTGGCCAGACTCGCGCTCTTCCACGTTGAACAGGGAAAATCACTAGAAGAAGCTGCCAACG
CATCACTGGGTCATATGAAGTCAAAGGTCAAGGGCGTAGGTGGTATCATCATGGTCAACAAAGCAGG
AGAGTGGGCGGTGAAGTGGACCTCCACATCCATGCCCTGGGCAGCGGCAAAGGATGGCAAGCTGCAC
TCGGGAATTGACTTCGGCGACACGAGCATCATTGACCTGTCCTAAACCCCCAGAAGCTGTATTCTAGA
AACTAGCTTGGAGGTGACGCACGGTCTCCTGGCGTGGAGACTTAGCTTCATCAATTAGATCTAGAAAT
AGAAAAATTCTGCAGCCTGTCACTCGTTTTGTTGCCTTGATCAGTGTCTGGTTGGGGGCGGGTTCCAAA
GTGTGTGAGAAATGCCCATGTTTAAGATAAAAATAAGACCAGTGAAGATGACCAACTGGACGGAGCCT
CCCTTATGAATAGTCCCCATTGTCTGAGATTAGAAAGAAGAAACAGCAAGATCTTAACAGTAAAGAAA
GGGCCTCGATGCAGGGAACGGTTTTGGAGTTGGGGGACCTACAGCAGTAACAGAAGCTTGTAGGTTGA
AGGGTGTTGGGGGGTTCGGAAGAAAAAGGCTCTGGGGCANGACCCAGTGCANGGCACAACAAGGCA
GGAAGGACCNAGTGGGACNCGGTGAAAGGGGACNCGGCANNCAGAAGTTAATGGGAACACTGGATG
GTGCANAACTCATTTCANAGTCCGGAAGAACNTTTGGCCAAAAGAAACCNNAAAAAACTNGGTTTGN
TTAAAAAAGNGAAAANTTNNTTTGACTTACCAANACAAACNGTTTTTCCAAAAGCTTGGA
TTNNNCANNNCTGGAGCTCCACCGCGGTGGCGGCCGCTCTAGAACTAGTGGATCCCCCGGGCTGCAGG
ATNTTGTNGAAGATGAGCGGGTGGCAGCGACAGAGCCAAAATCAAAGCCGGAACCTGAGGAGAGAGT
GTTCCAGAAGGAAGTGTNTCTTCNTACNTCACCACACCTGAAAGCANAGCCTGATCCAGCCACAGAGA
TGGCAGCTGAGTCACTGCCTTTTCCTTCGGGGCACTGTCCAGCTGGNAGCTGGAAGCCTGGTATGAGG
ACCTGCAGGAGGTGCTGTCCTCAAATGAAAATCGGGGCACCTGTGTTTCACCCCCTGGAAACANGGAG
GAAGAATCAAAAAGCTTCACCACTCTTGACCCCGCCTCTNTGGCTTGGNTTACTGAGGAGGCNGGACC
ACCAGAGGTCACACACACCTCCCAGAGCCTNTGCTCTCCAGAGTCCAGTCAGANCTCNCTGNCTCAGG
AGGAANAAGAGGAANACCNGGGAAGACCCAGAACCGGACACAGAGNGGCCAGTCCCCATCTCGGNC
TGGCAAGCNNCGCATGAAGGAAAAAGAACAAGAANATGAGAGGANTGTGGCNCAGCTTGCTGAAGA
AAATGAACCACTTCAACANGAAATCGAGCCCCTGACCAAGGAAAGTGGAGGCNCTCGCCGACTTTTA
ATGACCGGATGGTTAATCTGCCCCCAGCATTGAACCGGTTGGGACCCATCTCTTTCATTTTCAGGCCAC
TTTCTGANCCTTTGGTNGGAAGTTTTTACTGGCCNCCGTNNCCCNGNGCCCAANNNCNTACCCCAACC
CTTNTTTTTTNTTTAGGNNNGGGGAAAAAACCTTAGGGTNCAAAAAAAAAAAAAAAGGNCCCTAACG
NGNTNATTTAANANNGGCCCTTTTTTTTTTTTTTTGGCCCCTTTNCCNNNNAAGGNGNTTTTTNTNAAA
AAAAAAAAAAAAAAAAAAAAANAAGNGGGGGGGGGGGGGCCCCCCTTNCCCNNNTTTNNCCNNTTT
NNNNT
CAAAGCTGGAGCTCCACCGCGGTGGCGGCCGCTCTAGAACTAGTGGATCCCCCGGGCTGCAGGTGCTA
TAGTGTAAATTGAAAAAAGAATAGGTAATTTGCTTTGTACAGACACAAAAATGTAATGATGGTGCTAA
TTTCCTGGTAAAAATAAGCACTGCCAAGGGTTGAAGGACTGGTGACTTGAGAAACCTANAATCCTGTT
TGGGAGGAAGANATCTCATGTCTTAATTTCCTCGAGGTTCTGNTAGGANAAGGNAGAGCCTTGGTGAG
TCCTGCACCCGACCCTGGGGNGATCCNNTGCNNTCCAAAAGCNGAATACNCNCTNNGNGCNNANGAA
GGAATTCTTTGANTTTCTNAAGGCTGGTGNTACCTTGNCCACANCCTNCGTCCACACTGGCTGAACAG
GNTGGTANAAANCNCCANGNGNGGGANCTNCCTTANATACCTTNTGCCCCACTNCCTTTTNACTNTTC
TGGGANCACCTCTATTGGTTATGAAGACTAAGGATATTATTTCTTAAGGAAGTGGAAAGAGTTTATTTT
AATTAAAAATCCACAGCCTGGGCAGAAACATTTCCTATCAACATAAGCCCAAAATAGCACATGTGGNC
CCTTTTCTATAGNTACTTTCCTGTGACTCTCAAAACATNANGCTCTTTNTNGGCTTNANGNNTAACACN
TTTGGAAATCCCCCCAAACCTTTNTCAGATANNAAGGTGAGCCCCCGGGGCACGAAGCNCCAAAATG
ATTGGGGTCTGGGGGCTTNTAAACACCNCANNGGNGGGGGCAGNAAAAANAAGGCNGNNNTCCANN
ANNTTNAAAANNCCCCGCCTGCCCCCTGTGGCCCTNTTAGGNGGAAAAAAGTTGAAAAAANNGGGCG
AANTNGCCNCCNCCNCCANGAAGCCTCCTTGGAANAAACAACCNTTTTNTTAANNCNTTGGNNNNAA
AAAAA
NAANNCTGGAGCTCCACCGCGGTGGCGGCCGCTCTAGAACTAGTGGATCCCCGGGGCTGCAGGATTTC
CGGGACGGGGTCCAATTANAGTAACACTGGCCAACTTTTGAAATGTNTGNCAGCCCNCGGNTTCTCTT
GGGGGNTTTTNAATTACCCCNCCTGGGGTCTTNCNGGTGNANGNNNGGTNAAGGNCCNGGNCTTTTAA
NGGNNCANCNTTNATNACCCGGGNNGNAAAANCCGAANNCNAAAAAGNAGNAGNAGNAGNAGTTAA
ACNNCTTNATNTNTTTNGAAAGCNNTCTNGCCCTNGNANNGGNNGNNAGGGTNCCCNAAAAAAAGNG
AANCNTGGTNGTNGTTNACATTAAAATTANNNTNACCANTACCATTANNATGANTATTANATATTANA
TATTANANACACANTTTGNGATAGAGAGAANTTAACANAAAAGCTCCCCGTATAAGANAATNTNGTG
CTCAGATACTCTCCANCACAAAATGCGCACNNAATCNCGCCCNAATGTGGAAGANTCTCAACCCGNGT
GCACCCCAATANAAAGGNGACGANATNTNTTTAAAACANGAGAAANCACCCCGAACACNNNTANGA
NNNATNTCTGTTGTAANAGATTTNAGCACAAATGCNGCGCNCNTNTATAAAANGGTGTNTTCCTTCCC
CCCAGTGGTGGGCCCCCGNTTNTCATTTTTTNTTGAAGAAAAATTTTCCGNATTGACTAGACCAAGAA
GGCTNCTTNTNAANTNTTTTNTTANGTTGGGAGGAAAGACTTNTTTTTTAAAAAATAACTTTAAANCCA
GCTTTNTTTAAAAAAAANTTTCCGCCNTATTTTNTTTTTTTTAACAACAAGANAAACNNGCCTGGCCC
TTNTTTTTTATTGCAGGAGGGGGGNACTNTTCCCTTNCCCCCNTTGGTTNNANGTTGGTNCCACCNTTT
CTTTTTGTTCAANAAANTTTTTNTCCCAANNTGGCCTNTTTAN
NAANNCTGGAGCTCCACCGCGGTGGCGGCCGCTCTAGAACTAGTGGATCCCCCGGGCTGCAGGATTTC
CGGCACGGGGTCCAATTANAGTAACACTGGCCAACTTTTGAAATGTNTGNCAGCCCNCGGNTTCTCTT
GGGGGNTTTTNAATTACCCCNCCTGGGGTCTTNCNGGTGNANGNNNGGTNAAGGNCCNGGNCTTTTAA
NGGNNCANCNTTNATNACCCGGGNNGNAAAANCCGAANNCNAAAAAGNAGNAGNAGNAGNAGTTAA
ACNNCTTNATNTNTTTNGAAAGCNNTCTNGCCCTNGNANNGGNNGNNAGGGTNCCCNAAAAAAAGNG
AANCNTGGTNGTNGTTNACATTAAAATTANNNTNACCANTACCATTANNATGANTATTANATATTANA
TATTANANACACANTTTGNGATAGAGAGAANTTAACANAAAAGCTCCCCGTATAAGANAATNTNGTG
CTCAGATACTCTCCANCACAAAATGCGCACNNAATCNCGCCCNAATGTGGAAGANTCTCAACCCGNGT
GCACCCCAATANAAAGGNGACGANATNTNTTTAAAACANGAGAAANCACCCCGAACACNNNTANGA
NNNATNTCTGTTGTAANACATTTNAGCACAAATGCNGCGCNCNTNTATAAAANGGTGTNTTCCTTCCC
CCCAGTGGTGGGCCCCCGNTTNTCATTTTTTNTTGAAGAAAAATTTTCCGNATTGACTAGACCAAGAA
GGCTNCTTNTNAANTNTTTTNTTANGTTGGGAGGAAAGACTTNTTTTTTAAAAAATAACTTTAAANCCA
GCTTTNTTTAAAAAAAANTTTCCGCCNTATTTTNTTTTTTTTTAACAACAAGANAAACNNGCCTGGCCC
TTNTTTTTTATTGCAGGAGGGGGGNACTNTTCCCTTNCCCGCNTTGGTTNNANGTTGGTNCCACCNTTT
CTTTTTGTTCAANAAANTTTTTNTCCCAANNTGGCCTNTTTTAN
TTNANAAANNCTGGAGCTCCACCGCGGTGGCGGCCGCTCTAGAACTAGTGGATCCCCCGGGCTGCAGG
AATTCGGCACGAGGCTCGTGNGAGTGACNTCGNNTNTAAACGCTGCGTGGCAATCCCTGACNCACCGC
CGTGATGCCCAGGGAAGACAGGGCGACCTGGAAGTCCAACTACTTCCTTAAGATCATCCAACTTCTGG
ATGATTATCCAAAATGCTTCATTGTGGGAGCAGACAACGTGGGCTCCAAGCAGATGCAGCAGATCCGC
ATGTCCCTCCGCGGGAAGGCTGTGGTGCTGATGGGCAANAACACGATGATGCGCAAGGCCATCCGAG
GGCATCTGGAAAACAACCCGGCTCTGGAGAAACTGTTGCCTCACATCCGGGGGAATGTGGGCTTCGTG
TTCACCANGGAGGACCTCACTGATATCAGGGACATTGCTGCTGGCCAACAAGGTGCCAGCTGCCGCCC
GTGCTGGNGCCATANCGCCGTGTGAAGTCCCTTNNGNCAACAGAGAACACTTGGTNTGGGGNCCCGA
AAANACCTTCTTTTTCCCNGGTTTTNGGCANTACCACNAAAAATCTTCANGGGCCCCAATTGAAATCCT
GAGTGATGTGCAGCTGATTAAGACAGGAGACAANNNNNNNNNNCCNNNNNAANCCCCNCTTGNTGAA
CAATGCTGAAAAATTTNCCCCCTTTTTCTTTGGGGCTTGGNNATTCCANCANGGGGTTTGGAAAATGG
GNANNATTTTACAANCCCTGAAAGNGCTTTGACATTACCAAAGGAAAACTNTTGNANTTTCCCCNTTT
CTTGGGANGGGCGTNCCNCAAATGTTTTNCCCAGCGNGGNGNCCTTGCAAAATTGGGTTACCCCAAACC
CGGNGGNATTTTTGGNCCCCCCNTTTTTNTTAATTNAANGGGGNNCCAAACCCCCGNCCGNGGGTTTTT
TGNTNGGGGGNNAACTGATTAACNCCCTTTNCCCCTTTGGGTGN
NNCAAANCTGGAGCTCCCCGCGGTGGCGGCCGCTCTAGAACTAGTGGATCCCCCGGGCTGCAGGAATT
CGGCACGAGGATACTTCTTTTTTTTGAAAAATAATTGACTCTTTTATAAAGTATCTTTTAAATTAATAAT
GTGGATAAGTTTGAACAGAGAAAACATTTAATTAAAAATGATACTGCATCTTCTAAAAGGTTTTTTTCA
GATACATTTATAGTATATCAAGGGGCTTCCTCACTGGCTCAGCGGTAAAGAATCCACCTGCAATGCAG
GAGACATAGAAGACTGAAGTTCAATCCCAGGGTTGGAAAGATCCCCTGGAGTAGGAAGTNCCCACTCT
TGACAGGTTCCTCTCTTCCATGGACAGAGGAGCTTGGCGGGCAGCAGTTCATAGGGTTGCAGAGAGTC
AGACACAACTGAGCAGGACCACGAGCATAGTATATTAAACATGCTTTGGCTCATCAACAACCCATGGC
CAGTATGGTAGGCCAACTCTGATTTACAGGAAGATATCAAACTTTCGGCACAAAAGCTTTTTACAGTC
TTTAAGNGGTTTTCATTTTCATGTAAAAAATAAGTNGCATTTGATAGTTTATATTGGGGGCTTTTGANC
CTTANCTTGCTGGAGTTTGAAATTATTTCCTGACTCATAAGNATAATCTCTNTTAATATTAANCTATTAT
TGCTTGAANAACCAAGGTTACAGGTCTTTTCTCAATTTCTTCNACCTTNGGTNAACCCNCCAAAANGG
GANCCAGGGGATTTTGAANGGAAAAAAGGAANCNCTTGTTCTGGTTTTGGGTTAAACCCNTTTTTTTG
GGAGTTGGTTGCNTTAANTTGCCGGCAAGNNCCCNGGGGGCNTTACCTTTAANGGGGANCCCTTTGGN
CCCCCCANCCCAAAAAGGAATGGGNAANGAGNGNTCCCTGGAAAAATAAATTAAAANNANNNNCCC
N
TTNAAANNCNATCTGGAGCTCCACCNCGGTGGCGGCCGCTCTAGAACTAGTGGATCCCCCGGGCTGCA
GGAANAAAAAAAAAAAAAATTTTGGGGGGGGGNCCGGNNCCCNATTTTNCCTGTGGGGGGAGNAATN
TANAAGCGGTNCTCNNNTCCTGNTCCATGNTNTNAANAACNNTCNNGTCCCNTTGCCTGNACAGGAGG
AACCCTTGNNATATNCNCACCCTCCTTTGTCTGNTTAAGCATTANNCTGCTAATAGAGCGGNCTGACCT
GTTNATCNAGNGATACATCCTCGCGGTCAGGGATTCTTGGNGCTGGGTTAACGNNCCTGACTGNNTAT
TGCTGNTGAANCTGCACTACNATCTNNAGNATCAATANANCNTTNTTGTTACTTTCCACGCTNNCTGG
GGGNNANTGGGCCNTTCTCNTGCCTGTACCNCCTAGGGNNGGTAANAACATAAANANTTANATNNTTT
CCCTTGTGGCCTTACTTTCCATAATTTTCCNGGNCCNCCCTTGGNTTGNTTTGNTTCCCTTCATANTGGC
CNTTNGNGCTCCCTTTCCANNCNAGNGNAAAAGAAGGCCNCGNTGNTTAANAAATGACCCTTTTTNTT
CCGGCCANTTTTANCCNGGGGGAAAGGNANTCCNCNCCNNNACCCGGGGNAACNTNTTTTTTCCCAAN
TAANNNTAAAGNTNNNGGGNAGGTTTCCCCCCTTCCGGTNGGNCCCGAAATAAAAGGGGGTTNTTTCC
AANCCCANGGNAACCCAANCTNGTTGTNCCTTTCCTNGGNCCCCCCCTGANNGACTNGCTTAAAAANG
CNNCCNTNTNTTTTCCCCCCCCCCCCCCCCCCCCCCCCCCCTGGGGAAATNANNTCCCCCCCCCCCCCC
CCCCCCCCCNNTTCCNAANCNTTTCTTTTTANAAANAAAGGNNGGNAGCCNTTTTCCCTTTAATTTTCN
CCCCCTNTGGACANGNNAAAAGGGTTTTTTCTCTTTCCCCCCCCTTTTNTTTTTTTTTTTNGGAANCCCC
CNNCTNNGGGTTCCCCT
TTNNGCCTATNNGGAGNNGNATNNCAGNGGCGGCCGCTCTAGAACTAAGTGGATCCCCCGGGCTGCA
GGAATTCGGCACGAGGCTCGTGTGAGTGACATCGTCTTTAAACCCTGCGTGGCAATCCCTGACGCACC
GCCGTGATGCCCAGGGAAGACAGGGCGACCTGGAAGTCCAACTACTTCCTTAAGATCATCCAACTTGT
GGATGATTATCCAAAATGCTTCATTGTGGGAGCAGACAACGTGGGCTCCAAGCAGATGCAGCAGATCC
GCATGTCCCTNCGCGGGAAGGCTGTGGTGCTGATGGGCAAGAACACGATGATGGGCAAGGCCATCCG
AGGGCATCTGGAAAACAACCCGGGTCTGGAGAAACTGTTGCCTCACATCCGGGGGAATGTGGGCTTCG
TGTTCACCAAGGAGGACCTCACTGAGATCAGGGACATGCTGGTGGCCAACAAGGTGCCAGCTGCGGCC
CGTGCTGGTGCCATAGCGCCGTGTGAAGTCACTGTGCCAGCACAGAACACTGGTCTGGGGCCCGAGAA
GACCTTCTTCTTNCAGGCTTTAGGCATCACCACGAAGATCTTCAGGGGCACAAATTTGAAATCCTGAGT
GATGTGCAGCTGATTAAGACAGGAGACAAAGTAGGCGCCAGCCGAAGCCACGCTTGNTGAACATTGC
TGAACATTTTCCCCTTTTTCNTTNGGCTGGNCATTCCACAAGGNGGTTTGAAAAATGGCAGCANTNTAC
AACCCTGAANNGCTTNNCNTNNCNAAANGAAAANNNTTTGNATTTCCCCCTTTCTTGGGNGGGCGTNC
CCCAATGGNTTNCCNACCGGGGGNCCCTTCCAAAATTGGGTTANCCCTAAACCCGNGGGGATTTTGGG
NCCCCCATTTTTTTTTATTNAAANGGGGGNCCAAAAGCCCCGTTNCTTGGGNTTTTTNTTTT
GCCTACTGGAGCTNCACCGCNGTGGCGGGCGCTCTAGAACTAGTGGATCCCCCGGGCTGCAGGCACAC
GCCAGGAGCGCTGGGCTCCAGCCTTCTCACGCAGGTGGCTTCTCAAGGAGCTACCACCGTGTGTGAGC
TCCAGAGTAGGTGCAGATGTTTTCATCACATCTTCCACGTTGTTGACACTGTCTTACCTTCTCAAGTTCC
CGAAGTTCCCGAGAGCTTTCTCCTCTGGTATGCATTTTGTCTTCACAGCGCAGCTTGCAGTCCCTTGCTA
AAGATGATTATAAGCTCTGCATAGTGTTAAGCTTTATTGTGGTTATGTGTATGTTTCTTCTTCCTTTGTT
AAGCAGACCCACACCTTTCCAGGTCAAAGTACAGGATAGAGTGCTATCCTTCATCTTTACCATTTCTTT
TACTCCGTTTAAAGACTTCAGAAGTCTAATCCAGAGTTGAGCCAATTCAGAATTGACTGTAATTGAAC
ACAGGCTAAAAGTATTTATGGGGGGAGTGACTTTTAGCATGAGTTTTCTGATTTTTGTAGCTGCTAACC
TTTTAAATCTTTATTTGCAGTTCATGTAACAGTTGTCTTAAATTACATGATAAAGCAGTCCTGTTCAATT
TTTTTTTTAATGTGGCTTGTAGAATTTTTAATAAATGAGCTTANGTCTGTTTTTTCATGTGGAATGCAGA
TGGGTGCTATCAAAGCCTCTCCCCCATCACTATAGNGTAATAATATTATTACACCCCACTGAAATGGAT
TCAAAACTAANATTTTTTTTATNGTTAACCTTTATTGGTTCTTTCAAAAGNGGTGGCAAATGTACCGAN
TGGAAACCTGGTTCTTGGNCTGGAATATATAAACCCCTTCCAAANGGGTTNTTTTTGGGGGAGAAAAT
GGGGGGGGAGATTTTTCNTAAAANAAAGAAGGCNGGATCNGGTNATCNTCNANTTTTTACTTGGTCCC
TTAAATNTTTTTGGGGGGGGATNCCT
TTTAAGCCNATNTGGAGCTCCACCNCNGTGGCGGCCCGCTCTAGAACTAGTGGATCCCCCGGGCTTGN
AGGANTTNGGNTCGAGGCTTTTATTGAGACCACCCAAGGACCCCAGGGGACCCACACCTGGTGTNACG
GTATCCGTGGTTCTCAGCCCCANAATGGTGGGCTTGNTETCCCAGCCCCCTGCTGTCCCCACCAAGGGA
CAGGCCGNTCTCTGTGCCCTGCCTGGGATGGAGGACTCGATCATNAAATACNACCTCCTTTAAACATC
CANCCCTAGTCNTCCCCANCCCAGAGCCTGAGGGTCATTTGANAGGAAGTGAAAGTGGCNTGCNTCAA
TGGACAGNACTCANAAANCCCTTTTATAAAGGATATTTAATATCGNGNCCCCAGCTTNGTGGCNGGTT
TCANTTTTCTGGCCAGGNTAAAGTNTGGCTGGGCCTACNCTGCCNTGACATCTCTCTGACTGTTACCAC
ACGAGCTGAGTGCAGTGGANNAATGCGGCCTCTTTTTNTGCCAGGAGNGCAAAAGTGCCAAGCNGTT
GTTCNACACACNANNANATNNCGTNTTNCANANTGTGNGACCACCNACCAACTATGACATAACGACN
TCTGTAATNTNTNTAAAAAAAATATATTTTGGGNGCTGTCTTGAAAAAAAAAAAAAAAAAAANNTCNT
NNGGGGGGGGNNCNCNACCCTCNTTAATTCTGCTTTTNNAGNGGGNANTTATNTNAAAAATNTNTCTT
TNTNACCANTTTTTTANCANNNGTTTCNTTNNCNTNNGTGTNATTTTNTTNTNNNACNNACCNACNNTN
CANAATTNNNGCGTTTCCANCCTCTCNTNNNTCCNTCNNTNCNTGNTNNTTNTTATTTCNGAACATANT
CATNTTCTTCCTTCNCTTCNNNCATAACNTNTNTNCNCNNNTTNCTTNTTATTCTNNTTACANTCCTNA
ANCAAANNNANTANAATTTATNTGNNAANGNTCCCTATNTNTTNNNTANTACNTNTCANATTCCACNT
TATTANATCATNATTTNTCTCTTCCCTCANANNCCTNCCC
NAAAANCTGGAGCTCCACCGCGGTGGCGGCCGCTCTAGAACTAGTGGATCCCCCGGGCTGCAGGANTT
CGGCACGAGGCCCTCATCTCCGGACCACACTCAGGTTCCTGTTCTTTGGTGCCGAGTCCAGGGGATCC
ACCTCTGTCTCATCCGTGGCCTCTGCTGGTCCTGCCCAGAGGCCAGCACTCAGGAGACTCCTGCAGGG
GTTGCACCTGGCCCAGGGCACCTGCATTTGCAGATTTAGTTTCCCAATTCTCCTCTTGGTACCAAGTCG
CTTCTCCTACATCTGNTTAAANGCTGNATTCACCTNACGCTGGANCTNTGANCTCACACACTTGCCTCC
CGCGCGCCTCCCCCTNCCCAGCGTTTGACCCCGAAAGCCCTCTGAGGCAGANAACAGAAATGCCAGAN
CACCCGCCTTTTTATGGGGCACTCTTCANAAAGTCTCTGAGGCCCTGAAAAGAAGAACTACNTNATGG
AAACCATTTTGCANCCTNCANCACGGGCATTTATCTNAATAATCATTTCATTTAAAATGTGGGTCAAAC
ATATAAATCATATCTTGAAAGAAAGNATTTTGGTGCCACAGAAATCGCCCTGTGTTCTTACCACCTAAT
TTGAAAAATGTGTTTTGAAGTCANGAAAGAAAGCCAAACAAGCCTGNGGCCTGGGGTGTTTTTTCCCC
ACATGCNCCCTTTTCTTTTCNTGTGTNGCCCAAAAANNANNTCCCCCGCTCCTGNGCCAAAAGGNNGG
GGGGGAGNNCCNCACCCGTGAAGNTAGGNCACCTGTTCTGCGNATNTTAAAANCTCCCCTCCCTTTNC
ANAANAAAGGGGGTTGGGGAAACCCCGAAGGGNNGCCGGCNTCCTCAACCTGGTGCCTCATTACCCT
NGGGNCCCCNNCCGACCCCCTGGNGACAAGNGGTTTCANTTANNAACCCCNNTNTTTTTNGCCCTTAA
AAGCCC
NNNCNNNNCNNNNACNNNNCCTTNNTTANTNNNNTNNNTNNNCCTCTCNCNTANNNNTCCCTCANTTC
CTTNNNNTNCTTNTNTNTNANNGNNTNGNNATNAGTNGCTAATATCNCTTTTGAGNANNNCTGGAGCT
CCACGCGGTGGCGGCCGCTCTANAACTAGTGGATCCCCCGGGCTGCAGGAATTNCGGCACGNGGTTCA
ATTAAAGTAACNCTGGCAACTTTGAAAANGNGTGTACAGCCAACGGNTTCTCTTGNGGGCTTTNNNAA
TNACACCACCTGTGGTCTTACNGGNTGAAGTGTGGTTCACGGCCTGTGCATNTCATNGGACCAGCACT
TANTANCCGTGNANGAAGATGCAGAGTCNNAANAGGAGGAGGATNGANGANGTGAAACTCCTGAGA
ATATTGGAAAGCTGTTCTTGCCCCTGNANATNGGNTGCAAGGTTCCCCACAAATAAANTGAATCTTG
CTTGNTTGATGAAANATNAAGATNATGATTGACNATGACNATTGATTGNATGATGATCANANANGAN
NTATGATTGACCGATTNCTTGATTGANNNAANNCNTTNAGANTAAATNTTTNCANGTNNAAAGAAACT
CTGTACCCAGAATACNTGGTANNCNAAAAATNGCACCANNTNATCNGTTTTNATANAATNNNAGNTNA
CCTCAAAANTACTCGGTNNANNCNCCCANTTNATTCNAAAAAGGGNTCNCNTAANTTCCCTTTCCANT
AAANNTCCNNGGGAACAATAAACNNCCCGGNANNTNCCACCTTNAAAAGNNACCCTTANCCTCCTGG
NNNTCANNNACNTTTNAACAGNGCANNATAAGNTTGGCCNNGCCCATNGGTNATTNCAAAAACNTAN
AGGGNNGGGTTTTNCCTTTTTCCCCAAAANTNNNNTTNANTCCNCAANNTNNTTNTTCGNANTCCATT
GGNNNTAATNAAATNTGGNTTNNCCGGNANTNGACCTGGNACCCANGGGAANNGGCNAATTNNANA
ATAANCNTTCTAGGCNNAATNGGNAAAGNAAANACCCTTCTTTTAAACAATAAATATTCTCTTTNNAA
CCAGCNTTCNGGNAACAAANAANATCTTNCCANTTCTTANATTNCAAATTNTACTNNNNAANCCGANN
TCNGNANTNTACTCCNCANNGNGNNCNATTTNTGTCATNACGACCGGCGTAGGGAGTGCNCTAAATAT
TNNTTTCCTCGTNACACCCCGTNNNTAGTGAAATAANTANNGTAGTCGGNNCCACACGATCTANCTCT
NTNNGCTCCTNTNTAAANANTNTGTGGTCTNACTGTNCTNAACAANCNAGGGTNTGNTCG
NNNCNNNNCNNNNACNNNNCCTTNNTTANTNNNNTNNNTNNNCCTCTCNCNTANNNNTCCCTCANTTC
CTTNNNNTNCTTNTNTNTNANNGNNTNGNNATNAGTNGCTAATATCNCTTTTGAGNANNNCTGGAGCT
CCACGCGGTGGCGGCCGCTCTANAACTAGTGGATCCCCCGGGCTGCAGGAATTNCGGCACGNGGTTCA
ATTAAAGTAACNCTGGCAACTTTGAAAANGNGTGTACAGCCAACGGNTTCTCTTGNGGGCTTTNNNAA
TNACACCACCTGTGGTCTTACNGGNTGAAGTGTGGTTCACGGCCTGTGCATNTCATNGGACCAGCACT
TANTANCCGTGNANGAAGATGCAGAGTCNNAANAGGAGGAGGATNGANGANGTGAAACTCCTGAGA
ATATTTGGAAAGCTGTTCTTGCCCCTGNANATNGGNTGCAAGGTTCCCCACAAATAAANTGAATCTTG
CTTGNTTGATGAAANATNAAGATNATGATTGACNATGACNATTGATTGNATGATGATCANANANGAN
NTATGATTGACCGATTNCTTGATTGANNNAANNCNTTNAGANTAAATNTTTNCANGTNNAAAGAAACT
CTGTACCCAGAATACNTCCTANNCNAAAAATNGCACCANNTNATCNGTTTTNATANAATNNNAGNTNA
CCTCAAAANTACTCGGTNNANNCNCCCANTTNATTCNAAAAAGGGNTCNCNTAANTTCCCTTTCCANT
AAANNTCCNNGGGAACAATAAACNNCCCGGNANNTNCCACCTTNAAAAGNNACCCTTANCCTCCTGG
NNNTCANNNACNTTTNAAGAGNGCANNATAAGNTTGGCCNNGCCCATNGGTNATTNCAAAAACNTAN
AGGGNNGGGTTTTNCCTTTTTCCCCAAAANTNNNNTTNANTCCNCAANNTNNTTNTTCGNANTCCATT
GGNNNTAATNAAATNTGGNTTNNCCGGNANTNGACCTGGNACCCANGGGAANNGGCNAATTNNANA
ATAANCNTTCTAGGCNNAATNGGNAAAGNAAANACCCTTCTTTTAAACAATAAATATTCTCTTTNNAA
CCAGCNTTCNGGNAACAAANAANATCTTNCCANTTCTTANATTNCAAATTNTACTNNNNAANCCGANN
TCNGNANTNTACTCCNCANNGNGNNCNATTTNTGTCATNACGACCGGCGTAGGGAGTGCNCTAAATAT
TNNTTTCCTCGTNACACCCCGTNNNTAGTGAAATAANTANNGTAGTCGGNNCCACACGATCTANCTCT
NTNNGCTCCTNTNTAAANANTNTGTGGTCTNACTGTNCTNAACAANCNAGGGTNTGNTCG
TNNNCAAAGCTGGAGCTCGACCGCGGTGGCGGCCGCTCTAGAACTAGTGGATCCCCCGGGCTGCAGG
AATTCGGCACGAGGGCGAGACGAGCGAGGCAGGAACGTGGACGTCGGGTCTCTCGTTTGCACCGCGA
AGACACGTCTNCTGCAANGATGGTCANCGTNCCTAAANCCCGCAGGACTTTNTGTAANANGNGTGGA
ANGCNTCANCCTCNCAANGNGACCCAGTNTAANANGGGCAAANATTCCNTGTNTGCCCAGGGAAAGA
GGCNCTNTGATCGGAANCAAAGNGGNTNCGGNGGNCAANCCAANCCANTTTTCCGGANGAAGGNTAA
ANCCACCAANAAGATCGTGCTGANACTTGATTGCGTTGAGCCCACCTGCANATCCAANAGGATGCTGG
CCATTAANAGGNGCAGGCTTTTTGAACTTGGAGGANATAANAAGAGAANGGGCCAAGGGATCCAGTT
CTAAGCTTTGGAATCCTTTGGTNCTTANGTTTGGANAANAAAATGTTGAAGTTACAAAAAAAATTGCC
TGAATGANAATAAATNCCGCGATTTTCCTACTTNNNNNNNAAAAAAAAAAAAAACCTTNGGGGGGGG
GCCCGGGCCCCAATTTNCCCNTATAGGGNGNNNNNTNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNN
TNNNACAAAGCTGGAGCTCCCCGCGGTGGCGGCCGCTCTAGAACTAGTGGATCCCCCGGGCTGCAGG
AATTCGGCACGAGGGTCCTTCTGGCCCCGCTGGCAAAGACGGTCGNATTGGACAGCCTGGTGCAGTTG
GACCTGCTGGCATTCGTGGCTCTCAGGGTAGCCAAGGTCCTGCTGGCCCTCCTGGTCCCCCTGGCCCTC
CTGGACCTCCTGGCCCAAGTGGTGGTGGTTACGAGTTTGGTTTTGATGGAGACTTCTACAGGGCTGACC
AGCCTCGCTCACCAACTTCTCTCAGACCCAAGGATTATGAAGTTGATGCTACTCTGAAATCTCTCAACA
ACCAGATTGAGACCCTTTCTTACTCCAGAAGGCTCTAGGAAGAACCCAGCTCGCACATGCCGAGACTTG
AGACTCAGCCACCCAGAATGGAGCAGTGGTTACTACTGGATTGACCCTAACCAAGGATGTACTATGGA
TGCTATCAAAGTATACTGTGATTTCTCTACTGGCGAAACCTGCATCCGGCTCAACCTGAAGACATCCCA
GTCAAGAACTGGTACAGAAATTCCAAGGCCAANAACATGTCTTGGGTANGAAAAACTATGAACGGNG
GTACCCANTTTTGAATATAANGTTGAAGGANTACCNCCAAGGAAANGGCTACNCACTTTGCCTTTATGC
GTNTGCTGGGCAACCATGCCTTTTNAAACAGNCNCTTTCCAATTGCANNAACCNNNGTTGNANCCGTN
GAANGAAGGAAACTGGCAACCTGGAAAAAGGNTGGCATTTTTGCAAGGGATCCCATGGATGTCCAAA
CTTTGTGGCCCNNGGGCCAACCAGCANAATTNCCNTTACCCTGGTTNTTGGGAAAAGGGGTGGCTTTN
AAAAANAAAAAATGGAATGGGCCAAAAAAAAAATTTTTNNNNTTTTNAACCNNNCCNNGNCCCCTT
TTNANNAAAAGCTGGAGCTCCACCGCGGTGGCGGCCGCTCTAGAACTAGTGGATCCGCCGGGCTGCAG
GCTTTGCTGAGGGTCACATTGAGCTCCCAGCGACTTCAGGGGCATCTTTAGGAGTCAGCACTATGGCA
GAAGACATCCAGGCCAAAATCAAGAACTACCAGACTGCTCCTTTTGACAGCCGCTTCCCCAACCAGAA
CCAGACCAGGAACTGCTGGCAGAACTACCTGGACTTCCACCGCTGTGAGAAGGCAATGACCGCTAAA
GGAGGTGATGTCTCCGTGTGTGAATGGTACCGGCGTGTGTACAAGTCCCTCTGGCCCATATCCTGGGTG
TCAACTTGGGACGATCGCCGGGCAGAAGGCACGTTTCCTGGGAAGATCTGAACTGGCTCCATCCCACC
TCTTCTGTCCTCCGTCCTTCTCCCCAGGGTGGTGAAGGGGGACCGGGGTACATGGTGATCCCCACCCTG
GGATCCTGACTCATGGTATAACTAATAATAAATGCTCGTTGGAAACGTGAAAAAAAAAAAAAAAAAA
AANTTNGGGGGGGCCNGGGCCNCAAATTCCCCTTNTNNGGGGGTGGTTTAANNNNNNATNGNNNNCN
CNNNGNCCNNNGGNNCNCTTNNNTCNTACTANANTNGNNNNNNNNNNNNGNTNNNNCTNANGNNNN
NGNNNNATTNNNNTTTTTNNNNNNNNNNNNNNNNNNNNNNNNNNNNNNGNNNNNNNNTTTNNNNNN
CNNANANNNTNGGNNNNNAAANNNNNNNNTNGGNNACTTGGNTTTNCNNTNNNNNNNNANNNNNTN
NNNNTTTTGCCCAATANAAAAGCCNTTTANNNCCNNAAACCNNGNNNNTTTNCCNTTGNNTTTNNNNN
AAANNNNNAANTTNNNNNNNNCCNGGGGGNNNNGNNNAANTNNTTNNNNNNNCNNNNNNNNCNNN
NNNNNNNNNAAAANNNNNNAANCGGGNNCCCNNNNNNGGNNNNNC
TTNNANCCTATNTGGAGCTNCANNNCNGTGGCGGCCGCTCTAGAACTAGTGGATCCCCCGGGCTGCAG
GAATTCGGCACGAGGAATANNGAGAGTGCAGCTGGCATAAGCTGACCCANGAGNGCCACANAACGA
ATGCTCCTGCCTTCCCATGGAGAGTGCATGACCTCATGTNTGTGGAGCGCATTAGGGTCCTTGAGTAAA
ANGGAAAAAAAAAAAAAAGGTTCCTGNTGCTCTNATTTNTANNTACNCNGTNNACTNNNGTGGGNAA
ACCANGANTNNNTNGCCNGCTTNTTTCNNGAGTGNGTTTTTTGTTGNTCNACAAAANCNGGANGNTAT
NNGCTTNCNNCANAANTNGNNNNNAANGGAANACTATNCTTGNCCTATTATACCTNCANGAAACNG
NGACGGTGNAANTNATNCCTTTAGGNNTNNACTACACAAACTTGCCNAANNATNNCCTNCTCTGANA
AAGGAGGAATTCCNCNGGGATGNTGGANGNANCGNANGNTGGCCACCCCACANTATTTNNGANTACG
NTTNTNATGNGTCNTTCNTTGGTCTNGGAATNTNTCNCTNCCNCCAATTNTGTNGACACANNNCCATTT
CNATGTNNTNCNGTCCTTTTCCCTGCNCTNNTTTGGGTGAGGGGNTTCTTTAANGGGGGCCCTNTGCGG
NGGGAGCNCCTTCNCCCCCCCCCCTTGGACAAACTTTTTTTAAAAANGGCNAGGGNGGGCCTTTNTTN
TTTTNCCCTTTNACCCNGGGGGGNAATTNNGGNTGGTTTTTCCNGACCNTTNACCNNGGGNNNNTTTG
NANAACCNNCCCCCCCCCATAGGAGGGANAAAANCCNTTTCCCNTTTCCCANCCCTGGNGAAAAATTT
TTTTGGGCCCTTTTNGGNNCCCAAAAATTCCCTTTAAACCAAAAGTTGTTTTTTGGNAAACCCTTGANA
TTGCCCTTTG
NCNNNATCTGGAGCTCCACCGCGGTGGCGGCCGCTCTAGAACTAGTGGATCCCCCGGGCTGCAGGTGA
CAATGATCTTATAATATTCTTGACCCTTTTTATCATCTTTCAACTAAAAGTTTCAAAACACAACTTTTAT
CACAATCCAGAACTGACACCAACAAAAATATTAAAACAAAACACCCCTTGAGAAACAAAATGAACGA
AAATTTATTTACCTCTTTATTACCCCTGTAATTTTAGGTCTCCCTCTCGTAACCCTTATCGTACTATTCC
CAAGCCTACTATTCCCAACATCAAACCGACTAGTAAGCAATCGCTTTGTAACCCTCCAACAATGAATA
CTTCAACTTGTATCAAAACAAATAATGAGTATCCACAATTCTAAAGGACAAACATGAACATTAATATT
AATATCTCTGATCCTATTTATTGGATCAACAAACCTACTAGGCCTATTACCCCATTCATTCACACCAAC
AACACAACTATCAATAAACCTAGGCATAGCCATCCCCCTGTGAGCAGGAGCCGTAATTACAGGATTCC
GCAATAAAACTAAAGCATCACTTGCCCATTTCTTACCACAAGGAACACCCACTCCACTAATCCCAATA
CTAGTAATTATTGAAACTATCAGCCTTTTTATTCAACCTATAGCCCTCGCCGTGCGGTTAACAGCTAAC
ATCACTGCAGGGACACCTATTAATTCACCTAATCGGAGGGAGCTACACTTGCACTAATAAGCATTAGC
ACTTACAACAGGCTCTAATTACATTCACCATTCTAATCCTACTAACAATTCTAGAAGTTTGCAGTAGCT
ATAATCCCAAGCCTATGTATTCCTCTTCTAAGTCAGGCCTATATCTGNATGGACAACCCCATAATGGCC
NCACCCAAACTCATGGCTTATCATATAGTAAAACCCCAAGCCCCTTGACCTTTTTAAANGGAGCTTTGN
CTTGGCCCTTTTTAANAACATCCGGGCCTTAACCATTGGGAANTTCACCT
NCNATTTGGAGCTCCACCGCGGTGGCGGCCGCTCTAGAACTAGTGGATCCCCCGGGCTGCAGGAANTT
CGGCNCGAGGTTCCAGATGGCCCGAAGTCCACAGGCAGATTCAGAATCAGCTGGAAGAAATGCTGAA
GTCTTTTCACAACGAGCTGCTCACACAGCTGGAGCAGAAGGTGGAGCTGGACTCCAGGTACCTGAGTG
CTGCCCTGAAGAAATACCAGACGGAGCAGAGGAGCAAAGGTGACGCGCTGGACAAGTGCCAAGCTGA
GCTGAAGAAACTCCGCAAAAAGAGCCAAGGGAGCAAAAACCCCCAGAAGTACTCGGACAAGGAGCT
GCAGTACATCGATGCCATCGGCAACAAGCAGGGTGAGCTGGAGAGCTACGTGTCCGACGGCTACAAG
ACCGCCCTCACCGAGGAGAGGCGGAGGTTCTGCTTCCTGGTGGAGAAGCAGTGTGCCGTGGCCAANA
ACTCCGCCGTCTACCACTCCAAGGGCAAGGAGCTGCTGGCGCAAAAGCTGCCGCTATGGCAGCAGGCC
TGTGCCCGACCCCAACAAGATCCCCGACCGCGCCGTGCANCTTGATGCAGCAGATGGGCAACAGTAAC
GGCTCCATCCTCCCAGCGGTCTTTCGGCCTTCCAAGTCCAACCTGGTCATCTCAGACCCCATTCCAGGG
GCCAAAGCCCCTGCCGGTGCCCCNTGAACTTGGCCCNATTTGTGGGGGCGGNTGTCTGNCCAAGAAAA
ACCCCCCCCTTATTGAATGGGGGTTTCGGNCCCAACAGGGGANGACTTACCACCCCTTGGGCTTGACC
CGNAGGGCCCCCCCANCCCAAATTCCANTTCCCCCCCGNTNTTTTNAAAAACANGNTTGGGCNTTTTT
NTTTTNNAANATTNTTTCNGGGNANNAAAANNGGGCCCCNAAAAAAANNTTCCCCCCCNATTGAAA
AAAAAAANCTTTCTTTNTTNAACTTCATGGGCCCCGGCCGGAACNCAACT
NNANANCTGGAGCTCCACCGCGGTGGCGGCCGCTCTAGAACTAGTGGATCCCCCGGGCTGCAGGCTTC
TGTGGAANAANAAAGCTTTGGTCCACAACCTATTTCCCGACTAGANCANTGNGGNATNNATGCCANTG
ATGNGAAGAAATNGGAAGAANCTGGATTTCATACTGTANAGGCTGNTGCCTATGCACCNAAAAANGA
NCTACNNAATATTAAGGGGATNAGTNANNCCNNAGCTGATACAATTCTNACTGAGGCAGCTAAATTA
GTNCCNATGGGTTTNACCACTGCNAGTGAGTTNGNCCAAAGGCGATCTGAGATCATACAGATTACTAC
TGGCTCCAAANAACTTGACANACTGCTTCAAGGANGAATTGAGACTGGATCCATNACANAGATGTTTG
GAGAATTNCGAACTGGGAAGACCCANATCTGTCATACATTGGCTGGNACANGCCAACTCNCCATTGAC
CGACGTGGAGGTGAAGGAAAGGCCACTGTGCATTGNCACCNAAGGTACCTTTAGGCCTGAACGGCTG
CTATCAGTGGCTGAGAGATATGGCCTCTCTGGCAGAGANGTCCNNGATAATGNATCATATGCCCGANG
GNTCTCACAGACCACCNGACCCAGCTCCTTTATCNNGTNNNCTGCNCANNANGGGTTGAGTGNCAGGG
TATTGCACCTGGTTAATTTGTAGANAGGNGCCACTGNACCTCTANAGAAACGNGACTNTTTTNGGTTN
CANGTGAANNTTTTNAACCCANGGCATATTCCACTTTTGGTCGGGTNTTCTTGTAGGATTGCCTNTTTN
GCGAACTTGGGNNGGTNAAATTTNGGNGGGNNNCAAGGNGNGNATCCCCCCACCCCCNGGGGNGNG
NCCCCAGAGGNNTGNGNGCAAGCCCTNGTTTGNCTTCCNGANTNCNAAAAAAAACCCTTTTTNGGGG
GAAANTATTAATTTGTTC
NANNNCGANGNNNNNANNTNNNATANATTNNANANANNANNNTNTNNNNCNANCCNNCCNTCTNTC
TNTNNATATGGNATTCTTANGCANCTCNATATNGGAGNGNGGANCGAGGNAACATCTCTNTTGAGNC
CTCNAGNGAGTNGNATTACAGTNGNNNTNCTTCTNAAAACTAGCGGATCGCACANGNGNTGGNTAGG
ATACACCCNCANGAGGCCCTNNNTGNCCCTTTNAGCACGGTCTNGGGGCCGNGGGGCATCNNGGGNA
CACAGAGGNGGGGGACNTCTATAGCTNACNGCAANANTATNNTNNTAGCANAACTGTTTAGTCNCNC
ATGGCGNTNACTGNCNNATCACANTCCNATNNNNNANCTTTCTGNTCTNGNTNTTTNNGACNTATANN
TTGNNCTTTCGTAATNNTNTNNNNTNNCNNNNAGANNGANACNANATNNACTCNNCGCTNTCNTGAC
ACCCGANAGTCGATNAANGATAGNATTTTTNTNATACNTNNCGCNTNNTNNGNTCGNANACTTGNTCN
TNTAGAGTNTNTNCANNCNGACTTTTTNTANATANTNAATNTNNNNTAGTATNGTANANANTCACNCA
NGNNACNCCATNANANANNGCNTCNNNTNANTTCNGANNATACATGATANANGTNTCAATTGNTGCT
TGANTCTTCTNNATNNNANCNATCGAAGNNTTTNNACTATNNCTTTANANNNCCANNTNTNCACTTN
ANTTNNCNNNNATTAGNNNNNNNNNNATNTGAGNGTAATTNNNATCTNATTATTTNCNATATTNCTTC
NANCTNCTNNNANTNTGTNNNANGANGTTGTNNNNNTNNNCAANTCNNANANTNCCTTTCNTATTNNC
ATANTNATGNTGNCCAATCNTNNNACCTNAANCACNCNCTTATTNNCNTNCNGCTATGCTNCNACNTC
TANTNACTATANNACGTATCNTNCTTNTCGCCNCNTAGNCCNTATATATCTTCNTATTCTCNNCTTN
CCTNAANCNNNNTNNNATNTAGNATATCNCCTNCTGANCCTCCNANTGANTANTACCTCNACNATANC
ATACTNTGNCNTNANTCTCAATTGCANNGNNTANATCCNNNCNTATGAGAACATCCNACCTNNAAAA
GTNTTATANTTGNTATTGTANTGATCNATNTTATAANTNCNNTTTAATNTACTATGATNNNTATCCNTC
CGTTANGATGANCGNTTNNNTNTCNNACTGCGTATNTAGTTCCNAACTNNATNGATNACGTATNNANN
TCACTNTNNTGACTGNNNCNTACCTCNTNNTNCNCTNNTNTNGNANNGANATGAACNTCGCANTNTTA
NNTNGCNTTATCACTNTNATCTNAATGTATNTACNACNGNGNGATGTANTAANCTATNTTATNTTTCTA
TNTATNNTNGATCNATGACANNTNTNGGATNCTAGCNNTNNNNTNTACCTCNNACCGCTCGTTNCTAN
TNNNCNAACTATNGATNNCATNATTAGNACGTATNTACNTATGNTAANANCNATANGACTNTCATGAT
CTATTGTATNCATNNNNCANACTGAATNNNANNTNTNTG
NANNNCGANGNNNNNANNTNNNATANATTNNANANANNANNNTNTNNNNCNANCCNNCCNTCTNTC
TNTNNATATGGNATTCTTANGCANCTCNATATNGGAGNGNGGANCGAGGNAACATCTCTNTTTGAGNC
CTCNAGNGAGTNGNATTACAGTNGNNNTNCTTCTNAAAACTAGCGGATCCCACANGNGNTGGNTAGG
ATACACCCNCANGAGGCCCTNNNTGNCCCTTTNAGCACGGTCTNGGGGCCGNGGGGCATCNNGGGNA
CACAGACGNGGGGGACNTCTATAGCTNACNGCAANANTANNTNNTAGCANAACTGTTTAGTCNCNC
ATGGCGNTNACTGNCNNATCACANTCCNATNNNNNANCTTTCTGNTCTNGNTNTTTNNGACNTATANN
TTTGNNCTTCGTAATNNTNTNNNNTNNCNNNNAGANNGANACNANATNNACTCNNCGCTNTCNTGAC
ACCCGANAGTCGATNAANGATAGNATTTTTNTNATACNTNNGGCNTNNTNNGNTCGNANACTTGNTCN
TNTAGAGTNTNTNCANNCNGACTTTTTNTANATANTNAATNTNNNNTAGTATNGTANANANTCACNCA
NGNNACNCCATNANANANNGCNTCNNNTNANTTCNGANNATACATGATANANGTNTCAATTGNTGCT
TGANTCTTCTNNATNNNANCNATCGAAGNTTTTNNACTATNNCTTTANANNNCCANNTNTNTCACTTN
ANTTNNCNNNNATTAGNNNNNNNNNNATNTGAGNGTAATTNNNATCTNATTATTTNCNATATTNCTTC
NANCTNCTNNNANTNTGTNNNANGANGTTGTNNNNNTNNNCAANTCNNANANTNCCTTTCNTATTNNC
ATANTNATGNTGNCCAATCNTNNNACCTNAANCACNCNCTTATTNNCNTNCNGCTATGCTNCNACNTC
TANTNACTATANNACGTATCNTNCTTTNNTCGCCNCNTACNCCNTATATATCTTCNTATTCTNCTTN
CCTNAANCNNNNTNNNATNTAGNATATCNCCTNCTGANCCTCCNANTGANTANTACCTCNACNATANC
ATACTNTGNCNTNANTCTCAATTGCANNGNNTANATCCNNNCNTATGAGAACATCCNACCTNNAAAA
GTNTTATANTTGNTATTGTANTGATCNATNTTATAANTNCNNTTTAATNTACTATGATNNNTATCCNTC
CGTTANGATGANCGNTNNNTNTCNNACTGCGTATNTAGTTCCNAACTNNATNGATNACGTATNNANN
TCACTNTNNTGACTGNNNCNTACCTCNTNNTNCNCTNNTNTNGNANNGANATGAACNTCGCANTNTTA
NNTNGCNTTATCACTNTNATCTNAATGTATNTACNACNGNGNGATGTANTAANCTATNTTATNTTTCTA
TNTATNNTNGATCNATGACANNTNTNGGATNCTAGCNNTNNNNTNTACCTCNNACCGCTCGTTNCTAN
TNNNCNAACTATNGATNNCATNATTAGNACGTATNTACNTATGNTAANANCNATANGACTNTCATGAT
CTATTGTATNCATNNNNCANACTGAATNNNANNTNTNTG
TTNTANAAANNCTGGAGCTCCACCGCGGTGGCGGCCGCTCTAGAACTAGTGGATCCCCCGGGCTGCAGG
GGANNNTGNCTANCNCCCAGAANNGNCTNCANACGNGCGGNTCCGANCCNTACACTTTGGACGGAAN
TGTNCCAATGANTACNTGGAACTGNTGNACCNCGNNCTGCTGNCNCTGTGGANGCNANNCNCANATTT
GANGNNTCACTNACNGGNCACTCAAAGCGTGNGGCTGGATCATTTACTGACNTCATCNNGGTTGTNN
AGGCCATGAAGGNAACAGATTGGGNGNACNCACAGGACCCCAGAGTCATTGNTGAGAATGAACTCTT
GGGAGCCAAGNTTGCCATTGAGGTTGANNCCATTAAGCTGGACCACNTGATTCCTCGGACCANNCCTA
NGGAGGCGGNCAAGTNCTTGACTTTGANGACCATAATCCTGGAAACTTTCAATTTCCATCNAGGAAGN
CACCACCGCCTGGAGAAGGCTGCCTCATCTGNACAAAAGGACCTGGTGGNCCANGGGAAGGTGGTCN
CAATTCAGCCAACTCACTTGTTGATGGAAGTGGTCCCAAGGNCCTATTTTTTTTTGCCCGGATGGTGGA
TTGGGGCACCATCAACCTGNGNGAGGAAANNATTGCATTTTGGTCCAAGGGCCCCCCCTCCCTGGAA
AANGCTTNTTTTATTAAACCAAGGAAGGTTNNCGTTTTTNAAAANTNCCACNTTATTTGGAACCCTGCC
ANGNGCAAAGGGCCGGACCCCNGACTTTTGAAAGGCAATGGAAACCGGGTTTTAAGGGTTGGCCGGG
GAANCCCCATTNAAACCNGGNCCTTTNNACCAANCCTGGGGGAANGGNGGGCCCCNAAAGGGCTGGG
NNCCNTTTTTAAAAAAACCGGGNAAACCGGAANCCCTNGGGGGGTTNAAAGAAAAAAAAAGNGTCCG
GGGGGCANTTGNCCCCNAATTTTTTTTTTGCCCCAGGANA
AAANCTGGAGCTCCACCGCGGTGGCGGCCGCTCTAGAACTAGTGGATCCCCCGGGCTGCAGGCGTAG
GTTCAGNGGCGCTGAANCGGNNGCGTGGGGCTTTTAGGTTCTGTAGCTGGTCGCAATGGCAAGCAAAG
GAGGGTACATCGTGCTTGGGCTCCTGTTCCTGGCTGTCCTCTGCCAACTCGGTCACAGCCTGCAGTGCT
ACAGCTGTATTAACCAAGTTAACTGCACTTCAATCGTCAATTGTTCACATAATCAAGATGCTTGTCTCT
TCGTTAAGGCTGTGCCGCTAAAATTTTACCACCAGTGTTGGAAGTACGAAGAATGCAGTTTTGAGTTC
ATTGCAAAAGCCTTAGGGGAGAAGGAGCTCCACTATGACTGCTGCCAGAAGAACCTGTGTAACAAAA
GTGGCGGGAGGAGGGTATCCGANAAGATACTGCTGCTGCTCACCCTGCTGCTGGAGGCAGTCTGCCAC
TTTTATCGCTAAATCTATGCCAGGAGGGCTTCTCCTGAATATCCTGTTCTATCTATTCCTTCCTTCGGGG
GATGCTGGGTTCCAAANGCTTTTTATTTTCCAACTGGATCCTGGTTAATCNTTTCCCTGTGGGANANAC
TAAAACTAAGCTTGACCACTTGAATAATAGAGGAACTCAAAAAGACTNTGAANACCATNCTTTTGGCA
NAAAAAACTGGTTGAATTGAAAAAAGTTAAAAANGAACCGGGGTNANTTGAGTGANAAGACANGGTC
NTACTTTGCCCTTTGGGGGGGCAACNTTTCAAGGACCACTTGANGGACTTGTTTTTCAATNCTTAAAAC
GTTTCCCTTTGGGTNCCNGGGAAGGAAATAANGGGGAACAANCCTTTTTTNGGGGAACTTGGGGCCAG
GGGGGTTCNTTTTTAAAAAANTTTTTTTNNAA
TNNNNAAAAGCTGGAGCTCCACCGCGGTGGCGGCCGCTCTAGAACTAGTGGATCCCCCGGGCTGCAG
GAATTCGGCACGAGGCTGGACTACGGAAAGCGACTGGCGGAGGCCAGCAATTAGAAACTCTGGGTTC
TGGGTGCAAAAACCGAGTTAGGAGGCTCAGGCATGCTGCGTCCCAAGGCTTTGACCCAGGTGCTAAGC
CAGGCCAACACTGGAGGTGTTCAGAGCACCCTGCTGCTGAATAACGAAGGATCTCTGTTGGCCTACTC
CGGCTATGGGGATACAGACGCCCGGGTCACCGCGGCCATCGCCAGTAACATCTGGGCGGCCTACGACC
GGAACGGAAACCAAGCGTTTAATGAAGACAATCTCAAATTCATCCTCATGGACTGCATGGAGGGCCGT
GTANCCATCACGAGAGTGGCCAAGCTGCTACTGTGCATGTATGGCAAGGAGACCGTTGGNTTCGGAAT
GCTCAAGGCCAAGGCCGAGGCCTTGGTGCAGTATTTTGGAGGAGCCTCTCACCCAAGTAGCAGCATCA
TAATGGGCGTTGGTGGAAACCTGGGATTCANAAGAATGAGATGACCATGTGGTNAGGGCACGGCTTC
CCCGGGGAAAACCTTCCTGGACTTGTTGCAAGAAAATTGGAGTGGGGGGGACTTTGTTCTTNCCAANA
ATAAACTTCAACTGCTGTCTCAAANNNNNNNNNNNNNNNNNNNNNNNNNNNNNNNNNNNNNNNNNN
NNTTTNGGGGGGGGGGCCCGCCCCCCCNTTTTTTTCTTTTNNTNNGGGGGGNNNNNNNNNTTNANNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNN
TNANNCTATCTGGAGCTNCANCNCNGTGGCGGCCGCTCTAGAACTAGTGGATCCCCCGGGCTGCAGG
ANTTGCTGANGNNNACATTGAGCTNGCNAGCGACTTCAGGGGCATCTTTAGGAGTCAGNACTATGGCA
GAAGACATCCAGGCCAAAATCAAGAACTACCAGACTGCTCCTTTTGACAGCCGCTTCCCCAACCAGAA
CCAGACCAGGAACTGCTGGCAGAACTACCTGGACTTCCACCGCTGTGAGAAGGCAATGACCGCTAAA
GGAGGTGATGTCTCCGTGTGTGAATGGTACCGGCGTGTGTACAAGTCCCTCTGCCCCATATCCTGGGTG
TCAACTTGGGACGATCGCCGGGCAGAAGGCACGTTTCCTGGGAAGATCTGAACTGGCTCCATGCCACC
TCCTCCTGTCCTCCGTCCTTCTCCCCAGGGTGGTGAAGGGGGACCGGGGTACATGGTGATCCCCACCCT
GGGATCCTGACTCATGGTATAACTAATAATAAATGCTCGTTGGAAACGTGAAAAAAAAAAAAAAAAA
AATTNGGGGGGGGCCCGGGACCCAAATTCCCCNTTATNGNGNNNCTTTNTTAAANNNNNNCCCNGAT
GGGNGGNATGNNNNNCANCAACTANCTNTGTNAAGGNNTNTCCNNTTGCNNNCTTTTNNCANGGNNT
NNNNCCNTTNCCATNNANAAACCTTNATTNTNNTTCNNNNCNNNNCNNNNNNNTTNNNNTTNNAAAN
NNNCNNNNTGTTNNTTNNNGGNNNNNGGNNTNNNGNNCNNANCNNNNNNNTTTTNANNNNNNNN
NANNNNNNNTTTNNNGNTNNNCNNNNNNNCCNNNNNNAANNNNNNCNTNTNANAACCNNGNNNNA
ANGNCNNNNCCNNAAANNGTTTNAANCTTTTTNAANNAANNNNGNNNANNNTNNNNNCCCNNNNNN
NTTNNANNGNANAANAAANNTNNNNGG
GCCNATNTGGAGCTNCATCNCNGTGGCGGCCGCTCTAGAACTAGTGGATCCCCCGGGCTGCAGGAATT
CGGCACGAGGAGCTGAGAGTTCGAGAGCTACGGGACCCGAAAGACAGGGCTGGGCCCTGGCACCCTG
CGCGCCCCGACCCAACACTTAGGCCAGACCATGGGAACCCAAAAGCCTCGGATACTGCCCTGGCTGAT
ATCTGAGCTGGACCGAGGGGAGTTGGAGGGCGTGGCCTGGCTGGGCGAGAGCCGCACGCGTTTCCGC
ATCCCTTGGAAGCACGGCTTGCGGCAGGATGCCCAGCAGGAGGATTTCGGCATCTTCCAGGCCTGGGC
TGTAGCCAGTGGTGCCTATACTCCTGGGAAGGATAAGCCCGACCTGCCGACCTGGAAGAGGAATTTCC
GGTCTGCCCTGAACCGGAAGGAAGTGTTGCGTTTAGCGGAGGACGACAGCAAGGACTCCCAAGACCC
ACACAAAATCTATGAGTTTGTGAACTCAGGAGTCAGGGACATCCCTGAGCCAGATACCTCTCAAGACA
ATGGCAGACACAATACCTCTGATACCCAGGAAGACACTCTGGAGAAGTTACTGAGTGACATGGACTTG
AGCCCAGGAGGGCCCTCGAATCTGACTATGGCCTCTGAGAAGCCCCCTCAATTGTTTGCAGAGTGGCG
ACTCAAGACATCCCTTGCTCTTTGCCCAAACTCGGGACTCTTCTGAAAACCCCCTTGAACAGCTGTTGG
CAAACGANGAAGATTGGGAGTTCGAGGGGACTGCTTTTTACCGGGGCTTGNCAAATCTTTCCANCAAN
ACTGTTTTCTGCCCTGGGGGCCTGNNGNTTGGGNGGGATNANAAGCAGGGGACAGGATCTTGCCTGGG
CAANCTTATACNAATTGCCGGACCCTTGGANGTNCCTTGACAGAAAAANAACGNGANAANAATACNT
GNCAACGTGGTGCTTAACTGCTNGGGCCGGGGGGCTTGGCCTTGTGGCNG
NNCAAAGCTGGAGCTCCACCGCGGTGGCGGCCGCTCTAGAACTAGTGGATCCCCCGGGCTGCAGGGTT
ANGTTNGAGTCTGTATTGGACAAATAAGCACTATGCTTTTCAAATGATTGGAAAATCATTTTTATTTGC
CTCCTTTTTTTGACAGTGGTTTGACTTTTTGTTTGTTTAAGTAAAAATCACTAAACTTGTGCAATGGTAG
CATGGAAATTATCTGAGGTGTCTTGTATGATNGTGCTTTGGCCAGGGTGGACATAGTTCAAATTGTAA
NAGCTGAAGATAAAAGAGGAAGAAGGTATAGGTTCATGCTGCCTATTTTANAAGAGAACTTTCATTCTT
CAGAGCTACATAACATGATAATGCCACTGATTCATAAGGGGTTTAAATTAATTCTATGGGGTAGGACA
CCAGAATCATTAGTGTTCATTNTCATCTANGGTTTTTATCTCTCTAACGTTCTTGGCCTATTGAAACATT
TCAGTATACAAAAATACTGCGATGTTAATACCCAAGANAAAAGCCATCATANTGTGTATGCTGGTCAT
TTTGATCAGTGTGGCACAATTTTTTAAAAATAAACTATCANGCCCTTCAAAAAAAAAAAAAAAAAAAC
CNGGGGGGGGGGCCCGCCCCCCANTTNCNNNTTAANGGGGGNNNNNAAANNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNC
AAAAGCTGGAGCTCCACCGCGGTGGCGGCCGCTCTAGAACTAGTGGATCCCCCGGGCTGCAGGAATTC
GGCACGAGGCTTCGCTGGGGTCTGAGCCGAGCCGAGGCCTGGCTGCTCCCGCCGCCCACAAGCTGCTG
TCACCGGGCTCTGCACAGACAGGTAGAGGGCACCGAATTCCGGAGCGCCTACAGCCTGGACAAGCTG
TACCCGGAATCGCGGGGCGCGGACACCGCCTGGAGGGTCCCGGGCGACGCAAAGCAAGGCAACGATG
ACATTCCTGTAGATCGCTTGACGATATCTTACTGTCGGAGCAGTGGTCCTGGGGGCCAGAACGTTAAC
AAAGTGAACTCCAAGGCTGAAGTCAGGTTCCACCTGGCAAGCGCCGACTGGATCGCAGAGCCCGTGC
GGCAGAAGATGGCTCTCACGCACAAAAATAAGATCAACAGGGCGGGAGAGCTGATCCTCACGTCTGA
ATACAGCCGCTATCAGTTCCGAAACCTGGCAGATTGCCTCCAGAAAATTCGAGACATGATTGCCGAGG
CCAGCCAGCCAGCCACAGAGCCATNCAAAGAAGATGCTGCACTTCAGAAACTCAGGATAGAAAACAT
GAATCGCGAAAGGCTCCGAAAAAAGAGAATAAACTCTGGCATCAANAACNAGCAGGAGGGTCGGTAC
CGGACTTGAAAAGCACCCCCCTTCGAGCCAAGCAAAAANCCCTTNCANGGGCACCCCANGTGGGTCC
GGACACCAAGGANAACGGGTGTCTNGTTCGTGTCAACTGGTCGNCTTTTACNNNGGGAAACCGTCGCN
AAGAAAGGGCCCTTCNGGAAACCCAAANGTTGTTAAGCCCCTTGTNCCCCCAGGNNNTTTTTAAAGGG
GAATAACGTGGTTTTTAANACCCTTCGNTNATNCATNGGCCCTGGTTTTANTGGCAATAAAAATTCTTA
AN
GCCTATANNGAGTTGNATTACAGTGGCGGCCGCTCTAGAACTAGTGGATCCCCCGGGCTGNAGGAAAT
TCGGCACGAGGCCCTGGTGCCCCTTTAGCACGGGCTTGGGGCCGTGTCGCAACAGGGGCACACAGTGT
GGGGGACATCAAAGCGCCGNAAACCATGGCCTTTGCAAACCGTTCCGCCCCGCNTGGGGTCCTGGGGN
ACCCCCGCCNNTGGNAGCNTCNTGNTCNTGNTTTTTTGCNTNGGTNGGGTTNNACNTTCNAGGNCCNA
GGNTGNAAACNNGGAANNGGTTTTTTTNACNTAAANNGNNCANNNNANNTNGNGGAACNTGTCTTGG
NANAAGCNTNACNTNACNGTCTTCTTCCCAAGGGCCNGGGGGGNACNAAAAAAAAAGGCNTGCCCCC
NGGGGNGGAAGGCCAAATTGGNGAATAAAAACTTTTTTTTTATATAAGNATGTGGAACNTGAAGGCTT
GTGTGAACGGGNCCTGTTGNGCNCACCCAAATAAAAAANTGNANAACTGANATTCTTTTCATATANCA
AGANGTGCATNTTTTAAAGCGNAAANTGTANAANTTATACCCNNAGGGNNNACCCAGNNCTTTTATAG
AGCNNGAGANNTTTTTTTTTTTNTACGNCCCCTGTGGANATATNCACAAGGGAAATGACNNTTCTGTG
CACACCGAAAAAATTTTTTANAAAAAANCNCCAAAGGNGCGGNGGTGGAANANTCCNCAGGGGTGTT
CCTCTNATNNCCCNTNTTTTGNNGGGGAGGAAAGGACCCNTTTTNTANANAGGGCCCTTGTTANACCG
TTGTGGATNNTTTTGCCCTGCNTTAACNNGNGGGTTNTTTTCCGCCCCNNATGAACNTGGGTANTACTT
TNNNAAAATTAAAANANNGGNNTTTTTTTTNAANNAACCCCCCGCCNGNAANNTGNTNGGNNNGGNNTG
GCCCNNCCNNNNANAAAANATTTGTN
GCCTATANNGAGTTGNATTACAGTGGCGGCCGCTCTAGAAGTAGTGGATCCCCCGGGCTGNAGGAAAT
TCGGCACGAGGCCCTGGTGCCCCTTTAGCACGGGCTTGGGGCCGTGTCGCAACAGGGGCACACAGTGT
GGGGGACATCAAAGCGCCGNAAACCATGGCCTTGCAAACCGTTCCGCCCCGCNTGGGGTCCTGGGGN
ACCCCCGCCNNTGGNAGCNTCNTGNTCNTGNTTTTTTGCNTNGGTNGGGTTNNACNTTCNAGGNCCNA
GGNTGNAAACNNGGAANNGGTTTTTTTNACNTAAANNGNNCANNNNANNTNGNGGAACNTGTCTTGG
NANAAGCNTNACNTNACNGTCTTCTTCCCAAGGGCCNGGGGGGNACNAAAAAAAAAGGCNTGCCCCC
NGGGGNGGAAGGCCAAATTGGNGAATAAAAACTTTTTTTTTATATAAGNATGTGGAACNTGAAGGCTT
GTGTGAACGGGNCCTGTTGNGCNCACCCAAATAAAAAANTGNANAACTGANATTCTTTTCATATANCA
AGANGTGCATNTTTTAAAGCGNAAANTGTANAANTTATACCCNNAGGGNNNACCCACNNCTTTTATAG
AGCNNGAGANNTTTTTTTTTTTNTACGNCCCCTGTGGANATATNCACAAGGGAAATGACNNTTCTGTG
CACACCGAAAAAATTTTTTANAAAAAANCNCCAAAGGNGCGGNGGTGGAANANTCCNCAGGGGTGTT
CCTCTNATNNCCCNTNTTTTGNNGGGGAGGAAAGGACCCNTTTTNTANANAGGGCCCTTGTTANACCC
TTGTGGATNNTTTTGCCCTGCNTTAACNNGNGGGTTNTTTTCCGCCCCNNATGAACNTGGGTANTACTTT
TNNNAAAATTAAANANNGGNTTTTTTTTNAANNAACCCCCCCCCNGNAANNTGNTNGGNNNGGNNTG
GCCCNNCCNNNNANAAAANATTTGTN
GCCTATANNGAGTTGNATTACAGTGGCGGCCGCTCTAGAACTAGTGGATCCCCCGGGCTGNAGGAAAT
TCGGCACGAGGCCCTGGTGCCCCTTTAGCACGGGCTTGGGGCCGTGTCGCAACAGGGGCACACAGTGT
GGGGGACATCAAAGCGCCGNAAACCATGGCCTTGCAAACCGTTCCGCCCCGCNTGGGGTCCTGGGGN
ACCCCCGCCNNTGGNAGCNTCNTGNTCNTGNTTTTTTGCNTNGGTNGGGTTNNACNTTCNAGGNCCNA
GGNTGNAAACNNGGAANNGGTTTTTTTNACNTAAANNGNNCANNNNANNTNGNGGAACNTGTCTTGG
NANAAGCNTNACNTNACNGTCTTCTTCCCAAGGGCCNGGGGGGNACNAAAAAAAAAGGCNTGCCCCC
NGGGGNGGAAGGCCAAATTGGNGAATAAAAACTTTTTTTTTATATAAGNATGTGGAACNTGAAGGCTT
GTGTGAACGGGNCCTGTTGNGCNCACCCAAATAAAAAANTGNANAACTGANATTCTTTTCATATANCA
AGANGTGCATNTTTTAAAGCGNAAANTGTANAANTTATACCCNNAGGGNNNACCCACNNCTTTTATAG
AGCNNGAGANNTTTTTTTTTTTNTACGNCCCCTGTGGANATATNCACAAGGGAAATGAGNNTTCTGTG
CACACCGAAAAAATTTTTTANAAAAAANCNCCAAAGGNGCGGNGGTGGAANANTCCNCAGGGGTGTT
CCTCTNATNNCCCNTNTTTTGNNGGGGAGGAAAGGACCCNTTTTNTANANAGGGCCCTTGTTANACCC
TTGTGGATNNTTTTGCCCTGCNTTAACNNGNGGGTTNTTTTCCGCCCCNNATGAACNTGGGTANTACTT
TNNNAAAATTAAANANNGGNTTTTTTTTNAANNAACCCCCCCCCNGNAANNTGNTNGGNNNGGNNTG
GCCCNNCCNNNNANAAAANATTTGTN
TTNANCNCNATCTGGAGCTCCACCGCGGTGGCGGCCGCTCTAGAACTAGTGGATCCCCCGGGCTGCAG
GAATTCGGCACGAGGCAACGAGCAGGCCATCATCGACGTGCTCACGAAGAGAAGCAACGCACAGCGG
CAGCAGATCGCCAAGTCCTTCAAGGCTCAGTTTGGCAAGGATCTCATCGAGACCTTGAAGTCGGAGCT
GAGTGGCAAGTTTGAGAGGCTCATCATAGCCCTCATGTACCCTCCATACAGATACGAAGCCAAGGAGC
TATATGATGCCATGAAGGGCATAGGAACCAAAGAGGGTGTCATCATCGAAATTCTGGCTTCTCGGACG
AAGAACCAGCTGCAGGAGATAATGAAGGCATACGAGGAAGACTATGGGTCCAACCTGGAAGAAGAC
ATCAAAGCAGACACCAGCGGCTACCTGGAGAGGATTCTGGTGTGCCTTCTGCAGGGCAACAGANATG
ACTTGAGTGGGTATGTGGACCCANGATTGGNCCTGCAAGANGGACANGATCTGGACCCAGCGGGTGA
AAAAAATCTGNGGGACCGATGAAATGAAAGTTCATTACTNTTCTGGGGACNCCNCANCGCCCCCCACC
TTGATGANAAGTGTTTGANGGAATATTGANAAAANTGNCCACCCAGAACNNTTGNGGGCCCGCNTCA
ANAGGNGANAACCCNTGGGTTCCCTTGGAGGAGGNCNTTCTTTACAGGAATGGAAGGGCNCCCGGGA
ACCTTCCCCGGNTNCNTTTGNGGGGCGCCCNTTTCNTTTGCCATGAAAGGGAACCAGGGACTTTTGGTT
GGGGACCCCTGANAAAAAAAAATTCGTTTTCCCGGGGGNGGGNATTGGCCTTTAAATNTTTTTCAANN
AAATCANTTCNANNAAAAATTTTGCGGGGAAAAACCCTTTNNCNNNNTTTGTTTTTTGGGAAAAAACC
AANCNGGGGGANATACAAAAAANAGNCCCTTTTTTNAAC
NAAAACTGGAGCTCCACCGCGGTGGCGGCCGCTCTAGAACTAGTGGATCCCCCGGGCTGCAGGCTCTG
TTCCTAATGTGCATGGGGCTTTGGCCCCTCTGGCTATTCCGTCGGCAGCGGCAGCAGCAGCGGCAGCG
GGCCGGATCGCCATCCCAGGCCTGGCGGGGGCAGGCAACTCTGTCCTGCTGGTCAGCAATCTCAACCC
TGAGAGAGTCACACCCCAAAGCCTCTTTATTCTTTTCGGCGTGTATGGAGATGTGCAGCGTGTGAAGG
TCCTGTTCAACAAGAAGGAGAACGCCCTGGTGCAGATGGCCGATGGGAGCCAGGCACAGCTGGCCAT
GAGTCACCTCAATGGGCACAAGCTGCACGGAAAGCCAGTGCGCATACACTTNTCTAAGCACCAGAGC
GTGCAGCTGCCCCGCGAGGGCCAAGGAGGACCAGGNCTGACCAAGGACTATGGAACTCGCCCTTGCN
CCGTTTNAAAAANCTGGCTTCCAAAAACTTCCAGAACATCTTCCACCCTNGGCCANTNTGACTTTTCAA
CATCCCGCCCTCCTTNTTTTGAGGATAACCTCAAAATTNTTTTTTCCATAACCGGGGGGAATCGTCAAA
NGGGTTCAAGGTTNTTTCAAAAAGGANCCGNAAAATGGCGCTTGATNCCAAATGGGCTTTAAGNGGAA
GAAGGGCATTCCAGGGACTTTTTTGAACCTGGGCCAAACATTGAACTTGGGGGGANAAACCCNCCCNC
TTGGGGGGNGGTCCTTTTTCCAAATTCCCCCATTTTAAGGGCCGNCCANCCCAGGAAACAAAGCTTGG
CNGGGGANNCCCGGGGAANNAAATTTTCATTTNNTTTTCNNAANAAAAAAANCCCCTTTTTNAAAAAG
CNCGGTTGAAANNGGACCCTTTNAAANAAACCCAANAAATTTTNNTTTTTTTTTAAAAAAAAAAAAAA
CAANTTTAACCTGGTNTTTTNAAAAAAAAAAATTAAAAT
NAAAACTGGAGCTCCACCGCGGTGGCGGCCGCTCTAGAACTAGTGGATCCCCCGGGCTGCAGGCTCTG
TTCCTAATGTGCATGGGGCTTTGGCCCCTCTGGCTATTCCGTCGGCAGCGGCAGCAGCAGCGGCAGCG
GGCCGGATCGCCATCCCAGGCCTGGCGGGGGCAGGCAACTCTGTCCTGCTGGTCAGCAATCTCAACCC
TGAGAGAGTCACACCCCAAAGCCTCTTTATTCTTTTCGGCGTGTATGGAGATGTGCAGCGTGTGAAGG
TCCTGTTCAACAAGAAGGAGAACGCCCTGGTGCAGATGGCCGATGGGAGCCAGGCACAGCTGGCCAT
GAGTCACCTCAATGGGCACAAGCTGCACGGAAAGCCAGTGCGCATACACTTNTCTAAGCACCAGAGC
GTGCAGCTGCCCCGCGAGGGCCAAGGAGGACCAGGNCTGACCAAGGACTATGGAACTCGCCCTTGCN
CCGTTTNAAAAANCTGGCTTCCAAAAACTTCCAGAACATCTTCCACCCTNGGCCANTNTGACTTTTCAA
CATCCCGCCCTCCTTNTTTTGAGGATAACCTCAAAATTNTTTTTTCCATAACCGGGGGGAATCGTCAAA
NGGGTTCAAGGTTNTTTCAAAAAGGANCCGNAAAATGGCGCTTGATNCCAAATGGGCTTAAGNGGAA
GAAGGGCATTCCAGGGACTTTTTTGAACCTGGGCCAAACATTGAACTTGGGGGGANAAACCCNCCCNC
TTGGGGGGNGGTCCTTTTTCCAAATTCCCCCATTTTAAGGGCCGNCCANCCCAGGAAACAAACCTTGG
CNGGGGANNCCCGGGGAANNAAATTTTCATTTNNTTTTCNNAANAAAAAAANCCCCTTTTTNAAAAAG
CNCGGTTGAAANNGGACCCTTTNAAANAAACGCAANAAATTTTNNTTTTTTTTTAAAAAAAAAAAAAA
CAANTTAACCTGGTNTTTTNAAAAAAAAAAATAAAAT
AAAAGCTGGAGCTCCACCGCGGTGGCGGCCGCTCTAGAACTAGTGGATCCCCCGGGCTGCAGGATTCG
GCACGAGGCCAGTTTCCAGTTGTGTACCAAGGATGATTGCTTCAGTGTTAATATTCAGCCTCCTGTTGG
AGAACTGCTTTTACCTGTGGCCATGTGAGAGAAAGATTTTAAGAAGGAGCAAGGCATGCTCTCTGGAA
TGAATGAAACTTCCACCACGATTATTGCTGCCCCACAGAATTTTGCTTCCTCTGTGATCCTCAGAAGA
TAGTAAATGTAGCCAATGTGGGTGTAGTCCCTTCTGGACAAGATAATATACACAGGTTTGCAGCTAAA
ACTGTGCACAGTGGGTCATTGATGCTAGTCACAGTGGAACTGAAGGAAGGCTCTACAGCGCAGCTTAT
CATAAACACTGAGAAGACTGTGATTGGTTCTGTTCTGCTGCGAGAGCTGAAGCCTGTCCTGTCCCAGG
GGTAACCTGCTTACATCTGGACTTCAGAATCTGGCACACAACACAAGTCCCTGGCATCCACTGCTGCT
GCCTTTCATTTATAATAATAGCCCTTCCATCTGGCAGTGGGGGAAGAAGACACTCTTGACATTCTTGGC
TCCTGCTTTAAAAANGCTGGGGGTATTCTATCGNGAATGCAATACTTTTCCCCCTTTTCACTTTGCTAA
CCAAAGAACATACATTTTGCTGNCAGGTATCTCAACTTTCCTAAAATCCATTTGGCATTTTCTTNTGGCC
TGNTACNTTNTTTTNGGCCCTTCGNGGGTTNCCTTTNCTTGGTNTNAANCCNATGGANNGGACCAGGA
AACCGGGAANCCCTTGGGNTTTTTTGGGANAAAANTTTTTCNTTTTCCGCAGTAANGCCAAAAANTTA
NCCCTAAAAAATANNNGACCCTGGAACTNTTTCNNNTTGGNNTTTAAAAAAAAANGGGGGGNGTGGT
TNT
AAAGCTGGAGCTCCACCGCGGTGGCGGCCGCTCTAGAACTAGTGGATCCCCCGGGCTGCAGGAATTCG
GCACGAGGCAAAATGGTTGCCGGGTTATGTTAATTGGGAAGAAGAACAGTCCGGAGGAAGAAGCTGA
ACTAANNAAGCTGAANGATTTGGAGAAATCTGTGGAGAAGATAGCTGACCAGCTGGAAGAACTGAAT
AAAGACCTTGCTGGAATCCAGCAGGGTTTTCTGGCCAAGGATTTGCAAGCTGAAGCTCTCTGCAAACT
CGATAGGAGAGTAAAAGCTACAATTGAGCAGTTTATGAAGATCTTGGAGGAGATTGACACACTAATTC
TGCCAGAAAATTTCAAAGACAGTAGAATGAAAAGGAAGGGTTTGGTGAAAAGGATTCAGGCCTTCCT
AGCCGAGTGTGACACAGTGGAACAGAACATCTGCCAGGAGACAGAGCGACTGCAGTCCACAAACTTG
GCCCTTGCTGACTGAGGTGAGGCGGAGAAGGCCCGTGCTGCACTGAAGAACAGCCCCACCAGCCCTG
CCATCTCTGGAACAGAAGTTACCTGCCTTCCCCAGCGCTTGCTGGGAGCAATTGGCCCGTTGCCTTTTC
CTTCTCTTACAACCCACTCTCAATGAACAAGTGTCATTCCTTCCAATCTTTAACCTTCTATCAGCTTCTC
GTTCTGGCTCTGGGGGATCTGNGCTTGTCCANCANCCCCNCNTTGTTTGGGANAGGGCCCCATGACCA
AGTCTTTCCAAGTGGNTTGGANCCTTTGGGGGCTTTTNTTTNGGCCTGGGGAAAACTTNTTATGTATCC
CGGNGGAAGTTCTGAANTTTATTGGNCCNTTTTGGGCCTTCAAAAACCAAAAAGGNCCTTTTTGGTTTT
TATGAAACCCCTTAAANCCTGGNTTGGGT
GNAGNGCNNNNCTTNTCTTTNNANTTNNNNCTTNNNCCNCNNCNNNCNCNCTTCTTNTCTTNNCCTCA
CCTCNCTCCACCCGNCCNANGGNNNGNNCTATNTTNTNTNNCTCTTTANAGCCCNNTANGAGTCGCAT
ACANGGNNNNCCNTCNCATCTTTTCCTCCTCTTTTCGCACNCNACCTTCTCNNCNTTTCTTNTNTAANN
TATTNCCTNNCTCNTNCACCTCTTCNTNCNTNNNNNNNNNNGNNGNNGTTTCNATGTTNCAATTNNNN
TTAGTNNCCANANNNTCNTATCCANCANTCNNTCCATTNTTNAANACGNTNTCCCCTTNTTNTTTTNNT
GTNTATTNNNTNNNCCCTTCCANACAGTGNANNNCNCTNANTTTTNNNNTANTCTTCANATGTNTTA
GNTCNNNNNNNTTTANCNTCTANNNTANNATANNANTNACANNTATGNNTNNTNTTNTNTCNGNTTAA
NGGNCANNNTACCTTATNNTNTCTANNTCATNNAGTATTTTTTTCNTTTTNTTNGTAGTTNNNGCTC
GTATTNAGGTNNNCANTTCTCNCTCACNGNGTCATNTTTNTTNTTTNCGTTTNTAGTANGCNAATTT
NNNNTCNAGTNNANTNTTATTNNTNTNNTNNGNTTCNCTNTNATCTANTATAACTNNGNTCNTTGCTGT
TCNNNCNTNNAGCNTNCTCTNATCCCTNTCNTNTTTTTACTTTTNTTTNCCNCCTCCTCCTNCCNCNNTT
CTCNTNNTTCGNNCCTNTANCCCGCAGTNCCTCNCTNCNTATTTCAGCCAATNCTCNCNCCGTANNCCT
TCGTTCTNTCCTTTGNCTTCNTNACCNCNNNNTGTTTTTTTNNNATTNTCNNCTCTTANNNNNTCANAN
NNACTTTTGTTGTNTCCTNAATNNCTNNTNTACTNCCATCANNNNCCGTCCNTACTTGNACGTCNATNN
ANGTGANATTTNNNTNTNGNNNGGTCNCCTNTTTTNCGATANTNNNNACATTCGCNTTNTNTCACCTANT
CTTNTTTATTGNNCTGNNNNTNNACTNCNNCGCCNGTCAGTCAACNANTCTTATCTCCANTTNNNTNCG
CTTCGGNTNTCCTGTATNTTNCNTCTGNTNNTGCTNTNTATNATNCTCTNGANCTCTATATGCGTTTATC
ANTATATNTNCNTNATCGNCGTCTTATCNACNTTNGNTCNNTAACACCGTTNNTCATCNCNNNNNTTTC
TCTCTTTACNCNCACATTGTTNAACTNGCNGCNCGTCTNTGATTCTNCTGNTATNTGNGNGTTATNTNG
ACCTTCTCNTNATNATATCTTANATATGTCTNGATNTGCTNGACNTNCGCANNTGTNNTCCTNTCNNNN
NCGANTATCNTNTCNTCTTGCTNTNTGNTCNANANTNTNATCATGTTGTTNNGNCC
NNCAANNCTGGAGCTCCACCGCGGTGGCGGCCGCTCTAGAACTAGTNGGATCCCCCGGGCTGNAGGA
NTTCGGCNCGAGGCTCATGCTTATCATATAGTAAACCCAAGCCCTTGACCTCTTACAGGAGCTTTGTCT
GCCCTCTTAATAACATCCGGCCTAACCATGTGGATTTCACTTTANCTCAATGACCCTGNTAATAATNGG
CCTACCACCAAATATACTANCANTATACCAANGATGACGAGATGTTATCCGAGAAAGCACCTTCCAAG
GGCACCATACCCCAGCTGTCCAAAAAGGCCTCCGTTATGGAATAATTCTTTTTATTATCTCCGAAGTAC
TATTCTTACCGGATTTTTCTGAGCTTTATACCACTCAAGCCTCGCCCCCACCCCTGAACTAGGCGGGT
GNTGACCCCCAACAGGCATTCACCCACTAAACCCCCTANAAGTCCCACTGCTCAACACCTNTGTCCTA
TTGGCTTTCCGGAGTTTCTATTACCTGANCCCATCATAGTTTAATAAGAAGGGGACCGAAAGCATATATT
ACAAGCCCTATTTATCACCATCACATTAGGAGTCTACTTCACACTACTACAAGCCTNAGAATACTATGA
AGCACCTTTTACTATCTCCGACGGAGTTTACGGCTCAACTTTTTTTGAACCACANGCTTNCACGGGCCT
CACGTATNATTGGGCCCCCTTTTTAATGGCTGGTTCTTCCGCCAAATAAAATTTATTTTATTTTAACCNC
CCCTTNNGTTTTGAAACCGNTGCCTGGAACTGGACTTTTNGNAAACGAAGNCGGACTTTTCCCTTTAGG
GNNCTATCNAAGGGNGGGGGCTCCCAAAAAAAAAANNNNNNNNNNNNNNNNNNNNNNNNNNNNNN
NNNNNNNNNNNNNNNNNNNNNNNNNNNNNNGGGGGGGGGGGGGGGCCCCCCCCNNNNNNNNNNNN
NNNNNNNNNNNNNNNNNNNNNNNNAAAANC
NNCCCATAGAGAGGANNGGNGNGNCANANNANCGTCGACTGAGCCCNATNGGGAGNCGCANNACAG
GTGGCGGCCGCTATAGAACTAGNGGATCCCCCGGNCTGTANGAATCTCTGGCACGAGGCGANNTTTTT
TTTTCTTTNTTANCAANAAAAAANCCGNGNTNTTNTNTAAAAAAAANGGGGNGNCCCCNTCNCTTTCG
NATNGNNTNNCNNTGGGACCNCCNNNCNCNCNNCCTTTTTTGNTTAATGNGNCCTGGAGNCNTGCGNN
NGGGNAANATTATNNNNTTACTATTCANNNGGGNTCCTGNATTTNCTTCNTNANTTNCCNAGNAAANN
GCTNNNNTTTTTNTTTNGATCCNNNCCGCNTANAACTTTTTTNCNTTATANCGNNGNGGTGTCCATCAN
GGNNTTANNTANNNNATTANAGGGCNCCCCATNCTNGGTGNATCNNTGGGNNGTACNAAATTTNTCT
ACNCNNNTTAACNACNANNNGGGANGNTNNGGAGGGNATNANTNANTCCCNTTTACNAAATAGNCNT
NTNTTCTTTTTGTNNNACNTGNTNTTTTTCNNGNGNNAGCTNTNNTNCAAANNGGGNTTTNCTGANNTT
TNTAAANATGTNNNNTNGNGGGGGGGCTCCTNTANNCNNTCTTNNGANANNAGNNNCTTTNCNTTTNT
CNNCNNTNGGGGGNTGNANATTTTNTAAGNNCCGTGTCCNTCNCCNNNNTTCNGTTCCCCCNCTNNTG
NGTAATNGGCNCCCTNGCNCCCCNTTATTTNGCNCTNAGAAANNGTGGNNNNTNTNNANNCNGNAAC
NNGATGNNTTNTTCCNANNTNTNACNNNCNANTGTNCTCTAATTTNATCTTTTTNTGACCTANTTCNT
CTCCCTTGNAATACAANTCTNNNNNNNTCTTTGACNCNGTTTCACGNNCGNNAANTATANATANANNC
NCTTCATNTTTTCTTTTTCTTANTNCCCANTAAAANTNTGTNNCTTCTCCNTNNCCNTNTTNTNAACNAA
GACNAANGGAGAGGGGTCGCTTNTCTCNTNTTNNNCANACTATCNTTNATTCNANNGNNCNCNNCNAT
TTNTNTNGTNTNTAGTTGCACNNAGANANTNNTTAACTTNANNNNGCCCNCGNGTNATATAAANATNN
AGNANGTTCNTCNCTNTTTCACNANACCTTAGTTTACCNCTNCNTCNNTTNTAAANANATACGTCGCTN
CNGNNNGNTNNCCNCGCNACATNNGCTCTCTCCG
TGGAGGCCNCATAGGGGAGTCGTCATTTACAGGGGGGNGGNCNCTCNAGNAACGAGTGGGGATNTCNC
TAGNGGTTGGATGCATNCTCCCGATCTTTNGNGTTTTTNNNTATNCTANGNTCCANNAGAANNCTTNC
NAGNCGGGGGAATTTATATACTAGTAGTTTNACANNTACGNCCACNCNCTCGTCAGACTCGNNTTGNC
GCTGTNCNNTANTTNTCNNANNCTCNACANGANANTNGNNCNGTNGTTGTGCCNNTAGATTNNTATCT
NTAGAGNGNNGACNTGATGNCATGAGAGNCGGCTAGAAGTNAGCGAGGNGCGANANGTTNCANTTTA
NGCNNNCACGATGNACNANTNANCNAGANCNCGNAGANGANGCGNANNNGAAGTNGANNCGACTTC
NGAGTTNGNAGGGGNNGNNNNNGATGNTNCNGNNNCTATGNTCCTCGTGGGNCNCGNGAGNGNATC
GTGNTNNGNNTATGNNTNGNGANTCNCGAACGTNAGANNATNANNANCANGTGNNTAGACGGGTCA
GAGNNCNNAGATGATAANTNANGNNTCCGGANNNANTNCGGATATNANNNATANNTNATNTNNTTNT
GAANACTATATCTGNNCCNTGCTCGNNANNCNNTGTNCGNNNCGATCANGTTGNGAGACCNTNTANN
GCTGTATTGGGNTTNTNNTNNNGAGNNCNTNNNNCCCTAGGGGAAGNNTAGGNATNTATCNNGAGNN
NNNANAANTCNNNNGNTGNNTNAGAATNTNACNTNAGANNTANNNNTNANNCCACGATNNCNGTCG
AGNNTNCNNGNNNTGTATCNAGNCNNNNNTTTTTAGNNNNNTCGCNTTGTTCGNNGCNNNGNNNCNN
GNGGNGGNGGCTNTNCNNACACGNGGNNNTCTCTCATNTATNNGTTTTCCNTCTTTTANCANCTCTNA
ATCCCNCNGANCTTTATANCTCTNNCTNGTTACCNCGCATNANNNGCGNTCTATTTGGATCTCGANGA
GNTGGNATAGTACNTNNCACGTNCCCANGCNTNTGNANGGNNNNNNGCNATNNTCGGNATNANNCNG
AACTNTNNTATCNATNANACTGTNGAGAGAGCATAGNNNGNTNCATGNTNTCTGNANAANTTNCTTN
NCTGANTNNGANCGTACNTANNNGNGNGTAGTTANNNACNATGNANCNTANTANNNNTTTATANCGT
GAAAGATGTNGNACGNAGTNGGAANATNAGAGTANTANCTCGNNNGNTGGGTGANTTACNNTNGGAT
NCNTGNANGGCGANANNATGNCGNNCGTATGNNNNTGTATTATCGNTCGTNNATGGGATATCGNNAC
GCNCGGTANTACGTTNNTATAGTACNCTNTTCNNNGAGNNNCTTNCGTCNNGGNGCAGACGNAANTG
ANGAGGNANCCG
TGGAGGCCNCATAGGGGAGTCGTCATTACAGGGGGGNGGNCNCTCNAGNAACGAGTGGGGATNTCNC
TAGNGGTTGGATGCATNCTCCCGATCTTTNGNGTTTTTNNNTATNCTANGNTCCANNAGAANNCTTNC
NAGNCGGGGGAATTTATATACTAGTAGTTTNACANNTACGNCCACNCNCTCGTCAGACTCGNNTTCNC
GCTGTNCNNTANTTNTCNNANNCTCNACANGANANTNGNNCNGTNGTTGTGCCNNTAGATTNNTATCT
NTAGAGNGNNGACNTGATGNCATGAGAGNCGGCTAGAAGTNAGCGAGGNGCGANANGTTNCANTTTA
NGGNNNCACGATGNACNANTNANCNAGANCNCGNAGANGANGCGNANNNGAAGTNGANNCGACTTC
NGAGTTNGNAGGGGNNGNNNNNGATGNTNCNGNNNCTATGNTCCTCGTGGGNCNCGNGAGNGNATC
GTGNTNNGNNTATGNNTNGNGANTCNCGAACGTNAGANNATNANNANCANGTGNNTAGACGGGTCA
GAGNNCNNAGATGATAANTNANGNNTCCGGANNNANTNCGGATATNANNNATANNTNATNTNNTTNT
GAANACTATATCTGNNCCNTGCTCGNANNCNNTGTNCGNNNCGATCANGTTGNGAGACCNTNTANN
GGTGTATTGGGNTTNTNNTNNNGAGNNCNTNNNNCCCTAGGGGAAGNNTAGGNATNTATCNNGAGNN
NNNANAANTCNNNNGNTGNNTNACAATNTNACNTNAGANNTANNNNTNANNCCACGATNNCNGTCG
AGNNTNCNNGNNNTGTATCNAGNCNNNNNTTTTTAGNNNNNTCGCNTTGTTCGNNGCNNNGNNNCNN
GNGGNGGNGGCTNTNCNNACACGNGGNNNTCTCTCATNTATNNGTTTTCCNTCTTTTANCANCTCTNA
ATCCCNGNGANCTTTATANCTCTNNCTNGTTACCNCGCATNANNNGCGNTCTATTTGGATCTCGANGA
GNTCGNATAGTACNTNNCACGTNCCCANGCNTNTGNANGGNNNNNNGCNATNNTCGGNATNANNCNG
AACTNTNNTATCNATNANACTGTNGAGAGAGCATAGNNNGNTNCATGNTNTCTGNANAANTTNCTTN
NCTGANTNNGANCGTACNTANNGNGNGTAGTTANNNACNATGNANCNTANTANNNNTTTATANCGT
GAAAGATGTNGNACGNAGTNGGAANATNAGAGTANTANCTCGNNNGNTGGGTGANTTACNNTNGGAT
NCNTGNANGGCGANANNATGNCGNNCGTATGNNNNTGTATTATCGNTCGTNNATGGGATATCGNNAC
GCNCGGTANTACGTTNNTATAGTACNCTNTTCNNNGAGNNNCTTNCGTCNNGGNGCAGACGNAANTG
ANGAGGNANCCG
TNNANNANNNTCTGGAGCTCCACCGCGGTGGCGGCCGCTCTAGAACTAGTGGATCCCCCGGGCTGCAG
GAATTCGGCACGAGGGCANGACCACGAGGANAGAAGGCTGCACCATCTGANGACCCTGCANGGTGAG
AGAATAGANCATTACAAGGCNNTGGAANAAGCACTNAACTCGCCGATCCTTTGTCTGGGGCCGAGAT
GGNAGGTGGAGCCTCAGGAAGCCTCCAGAGCTGTTCACCAGANGGATGCTGGCGCANNAGAAAAAAG
TTCCTGGCTCAGTTCACCACGCACCAGCGGACTCGCCGTGACACGCGGAAACNGAAAGGCGAGGGTC
ATGGACCATCTGGAAGCCCAGCTTGAGACCCANCTACAGGAAGCTGAACAGAACTNCATCTGAAATCT
TGGCAGCATTGGCCCGGGTACCTCTTGCCGAAAGCTAACTATTTTCGAGTAAACGTGGTCTCTCAGCA
GAGAACCCCCTGAGGACTAAANGGGAACNAAGCCTTCTACCCCGATGAGAGAGGGGACACTTGGGGN
TCCCCAGGATGATGACCCTGCCTGGGGGGCCCCACGTTCAGGATCGCTCAACAAAAGGCTGATGCCA
GCAAGAAAGTGAAGTTGGTGATGGCTANAAATTCCATAAATATGCTTANAAANAAGAAGCCATCTGT
AACTTTAAGGCTTGCTCAAGACANGNCCTTGAAGACCCGGGCNTTNTNTTGAAGNCCACNTTGCCCCT
TCCCCCAGGANGAATTTGCCCNCCCCAAGGGTANGGGGGGACAAATGGGGGTCNTTTCCTTTTTGCCC
CCCCGGCTTTGTGCCAGGGCTTGGGAAAAATGGGTTGGCCAAAATTNCCCAACCAAAAGGNCTTTAAA
AAATTANGGGNGGANCCCTTCTTGCCTTTTGAAAAATGACCAAGCTTNCCCGGGGTTNGGGAAAAACC
ATTNTTTTTNAAGGNTTTTTGGCNTCCCCCCCGGGTTTTTNANAAAACC
NCNACTGGAGCTCCACCGCGGTGGCGGCCGCTCTAGAACTAGTGGATCCCCCGGGCTGCAGGCTCGAG
TTTTTTTTTTTTTNTTTTTNGGGTCCCNGNNCNANGNNCTTNNGGGGNNTTTNCCCGGGTNNAAAAANG
GGGGTTTTTTTGGGNACCNCTTNGNNTTTCCAANNNNNACGGGCCCCCCCCNNNNAANCCCNTGGNNG
GGGGGGGGGNGGNNNCCCCNANNCNGGNCNGGGNTNNTNAAAAGGCCNCCNNCNNAAAAAAAANCC
NTTTTNGGGGTTNNNCCNGGGGGGCNAAANTNNTTAAANCCCNTTNNAAAAANTTTNGTNNTNGAAA
NGGAANNNNCCNGGGTTNNNTTTNNAANGGCCCCCCCCCCGGNCCNTTTNAAAANNTTNNTNNTNNT
AAAAANAAANCCCCNNAAANNNNGNGGGGNGGCCNAAAANCCNTTNNNNNCCCNAAAAANNNNNNT
GAAACCCCNNCCCCACTTANCCAAATTTTTTCCNNTTTAAGGNNAACCGGANAAAATTTGGGAATTT
TNGNGCCCNCCGAAANTNNTTTCGGANGGGGAAGGGGGAAAAACCTTTGGGGGGNGGGGTTTTTTTTT
GGGGCCCCNATTNGGGTTTAGGAAAAANTTTTTTNTTCCCTGGAANGGCCCCTTTTTNNAGGCCCCGN
ACCNTTTTTAANGGGNNNGGGGCCCCAATTTTTANAAAAAAAANNANTTGNGGTNAAACCNTTTGAA
ACCNCAAATTTTCCCCCTTTTGGGGAANNNCTTTTTTAAANGGGCNCCCCCNCTTTCANAAAAATT
TNGGGCTTTTTTTTTTGGGGAAATTCCTTTTANNGGGNTNTTTTGGGAAAAAAAAAAAAANTNTCCCAT
TTTNCCCAAAAGGGAAAAAAAGNCCCCCCNGGGGGGGAATTTNNCNAAANTTTNGGGGGGGGGNGG
NNAAAAAACNGGNGTTAANGAGGGG
NAAAANCTGGAGCTCCACCGCGGTGGCGGGCGCTCTAGAACTAGTGGATCCCCCGGGCTGCAGGAATT
CGGCACGAGGGCGGTCACCCTCGGTTCCAGGCACCGCACCGGGCTAGCGGCAACATCGCGTCCATGGC
GACCACGGCGGCTCCGTCATCTTTGGCCCTCAGGGCCTCGAGCCCGGCCGCGACCCCCAGTTCGTACG
GGGTCTTCTGCAAGGGGCTTTCCCGCACCCTGCTCGCCTTCTTCGAGCTGGCCTGGCAGCTGCGCATGA
ACTTCCCGTATTTCTACATCGCGGGCTCCGTCGTTCTCAACATCCGCTTGCAGGTACATTTTTAGAGGC
CTGGGGATGCCGCCGGCGTTCCGGGCCTGCGTTGTGGCCGACTCCCAGGAGGCGCCGCGGCACCGTGG
AGCTTGGGGCGAGCAGTCCCGCTGTCTCGCGAGAGTGCTTGGTCTCCCTCCACCGGTGCGGTCTCGCCT
CTCGGNCGTCTGCTCTTCTTCCGATCCCCTGAACTGGCACCGAAAACCTCCTGAGTGTANGAAGANGA
AATACGCGATTNCCGGGAACCGAGAAACTTCAGTTAAAAACCCCACTACAATATTTAAAGAAGCTGTG
CATTCCTTTAAAAACGGGAACGAAACAGGATGTANTTATAACAANGGGAAAGGCTGGTCCAAAACTT
TTAAGGACAAGAAATGNACAAACCTTAANGGGCCGTATTTATNNAAATAGGAAAANTTTCGGGGAAN
AATGAAAACTGGGATTNTTAAACCGGGGATCAGTCCNNAANCCTGGAANAATTTTAANGGGAATTTG
CCGNGGGAANGGNNAAAAANTTTTTTTTTNTTCCCCAAACCGGAAAAACTTTTTGACGAATTCNNAA
AANNTTTAGGGGAAAAAAAAAAAAAAAAAAAAAAAATTTGGGGGGGGGGGGCCGGGNCCCAAATTN
CCNTTTNNNNNNNNNNNNNNNNNNNNNNNNNNNNNNNNA
NAAAAGGTGGAGCTCCACCGCGGTGGCGGCCGCTCTAGAACTAGTGGATCCCCCGGGCTGCAGGATTC
GGCACGAGGCCAGTTTCCAGTTGTGTACCAAGGATGATTGCTTCAGTGTTAATATTCAGCCTCCTGTTG
GAGAACTGCTTTTACCTGTGGCCATGTCAGAGAAAGATTTTAAGAAGGAGCAAGGCATGCTCTCTGGA
ATGAATGAAACTTCCACCACGATTATTGCTGCCCCACAGAATTTTGCTTCCTCTGTGATCCTTCAGAAG
ATAGTAAATGTAGCCAATGTGGGTGTAGTCCCTTCTGGACAAGATAATATACACAGGTTTGCAGCTAA
AACTGTGCACAGTGGGTCATTGATGCTAGTCACAGTGGAACTGAAGGAAGGCTCTACAGCGCAGCTTA
TCATAAACACTGAGAAGACTGTGATNGGTTCTGTTCTGCTGCCGAGAGCTGAAGCCTGTCCTGTCCCA
CGGGTAACCTGCTTACATCTTGGACTTCAGAATCTGCACACAACACAAGTGCCTGGCATCCACTGCTGC
TGCCTTTCATTTATAATAATAAGCCCTTCCATCTGGCAGTGGGGGAAANAANACACTCTTGACATTCTT
GGCTCCTGCTTTAAAAAGGCTGGGGNGNATCTTATCGGGAATGCCATACTTTTCCCCCTTTTCCNTTTG
CTAACCCAAANAACATACATTTTGCTTGGCAAGTTATCTTCAACTTNCCTAAATCCANTTTGGCANTTT
TCTNTGGGCCCNGGTACNTTNTTTTNGGNCCCTTGNGGGGTTCCCTTTNCCTNGGTTNAACAATGGATG
GGACTTGGAACCNGGGAAACCTTTGGGGTTTTTTTGGGNNAAAAATTTTTTCNTTTTCCGGGGTAAGGC
CNAAAAATTTGNCCCTAAAAAATTTNNNNNACCTTGGATTCTTTTTA
GCCTATAGTGAGTCGTATTACANGGGGCGGCCGCTCTAGAACTAGTGGATCCCCCGGGCTGCAGGAAT
TCGGCACGAGGAAAAGATCCAAATAAAGAAAAACAAGCATTTGTGAAAACAGTTAAAGATCAAGCTT
CTGAAATTCAGGTGACAATGATGCTCACCGAAAGTTGTAAGCTCCGAGGTCTTCATCACAGAAATCTT
CTTCCTATTACCAATGTGTGTATAGAAGAAGGAGAAAAGCCCATGGTGATATTGCCTTACATGAATTG
GGGGAATCTTAAATTGTTTTTACGGCAGTGCAAATTAGTAGAGGCCAATAATCCGCAGGCAATTTCTC
AACAAGACCTGGTCCACATGGCTATTCAGATTGGCTGTGGAATGAGCTATCTGGCCAGAAGGGAAGTC
ATCCACAAAGACCTGGCTGCTAGGAACTGTGTCATTGATGACACACTTCAAGTTAAGATCACAGACAA
TGCCCTCTCTAGAGACTTGTTCCCCATGGACTATCATTGTTTGGGAGACAATGAAAACAGGCCGGTTA
GGTGGATGGCTCTTGAAAGTCTGGTTAATAATGAGTTCTCCAGCGCTAGTGATGTGTGGGCCTTTGGA
GTGACGCTGTGGGGAACTCATGACTCTGGGCCAGACACCCTACGTGGACATCGACCCGTTTGAGATGG
CCCGCATACCTTGAAAGATGGTTACCCGGATAGCCCCANCCAAATCCAACTTGTCCTGGATGAACTAA
TTTGCCGTGATGGGCTTGTTGCTGGGCCCTTTAAAATCCNGGAGGAAGAGNCCCAANGTTCCAGCACC
TTGGTCCANGGGTCTTCCANAGTTCCCTTGCCNGNCCTTGGNGGGCCTTNTNNNTTGACTNTGNNTGGN
NNAATCTTTCCNAGCGGTCCANGAANGAATGGGGGCTTCCCGNGCCTTNTTNGGANCGAAATCNTNGA
AGNCNNNTTNGTTTNCGCCNNNCCCACANAAANCCCNTTTTTTTTTNCCAAAAAANCTNNNCNTTTAN
AAACG
GNCCCTANTANGTGNGGTCGGTAATTAANNNGGGGGNTTCTCATATCTNCATCNTNNGTGGGATTCCN
NNTNGGGTCTNCCANGNNATNCNTTNTACNATAAGTNTNGTTGNNNTNTTNGTCGANACTATNTNTGN
GAAGACGANATGNNTAGGGNCTNNATATNNTCNGANANAGTNNANANANTGTNTNGTAGCTAGNGA
ACCATNGANNNGCGCNNTNNGGTNCNNGTCANTCGAGANGCACGTNGNTGATACNNNAGAGNGNGA
CGNNANNNTNACNAGTCTNCATNAGGNCNAGTAGANNGACATNGNAGTGAGAGATATANNATTACA
GCGACNTCNGANNCNNGTTCNTTAGTNGANAGANNCNGTNNGNCANGAGNGATGGANAGNGNGAGA
GNGNGNCGNTGATGTGAGNATAANNNGNNACNCCNTCGNNANCGCNACANNNNTGCNGCNANACAC
ANTNANAGGGCAGNANACNAGAGNTTNCNATACNNTGNGNCTGNTGNGNGNNNGAGNTGNGTCAGN
ANANTNNCNGATCCNGACGNNNNANNGNNTGATAACTCGTNANNNAGTNCCNAGNGCNCNTGTTNNA
GTTATACCNAGTCNAGAGGTGNTCGACANCGGNAGGTGANNCNNANNCTNCCNTGAGGAGNNACATN
CTNTCNCNNNNTNGTTTNCNNGCTATNCGCNAGNTANNCATCNNNAANTNGTANACGNACACNNGNA
NATATGNGNTNNNANTGNTNGTAGNGNGNCNNNACNNCNNTCANGNGANNNNTGAGGCNANNANTA
TCGNNTGGACCNNNNATANNANGNTGANCTCCNCGATTNCNTANACACCAGTAGGNACGNTNTGNNA
NTNGNGTNNTGAGANNNNGNAGTNATNTNCGTGCTNNCACNATTTTCNNNCGTAGGAGNCTGNNNNT
GNNNTCCGAGNTATNCNNNTTTNNNCNGNNGTAGTGTGTAGCGGATGCNANACAGNTTCGCGCNTTC
NNANNAGNNNTANNTCGNNGANTNAGGNGCTCTAGNGGNTGTCGACNNNCGAGNCCTCNCNNANNC
NAAGNNTNNNTCNNCATNACNGTTNGCGNCGTAGNNNATGATCGNGNCCTTTGATCNNNCNAGTNNT
TANGATATNTCNTNACATATCANNCGCATCNCNTCGATGTNNNCNTAATTNATCCCCNTTNNNANGAT
NCGTGNTNNNNAANGTAATGCGTCGNGNNNCANNNCATNANAATTGNTCTNTCCGNNTNNAANCATN
CNNACGCTACGNCTTNTNACNNCNNTNTGTCTNCGACGNNACGGNATNCTTCCTNATCTNACNNGACT
CNNTTTNTGACANATCNCNTNCNNCNTNATGTCCG
NCTCNANAGNNNNNNGNNAGNGCGAAGGACCACTNTTNGTTNTANCCCCTTACTGANNCCNNNTTAC
AGAGGNTNATCTGNNTTCTCAGTNAANCANNCANTNCTACTNNAATTNCNAAATATNNTCCAGNACTT
CTANTCATTANGCTGGGTANTNNTAAAACTTCATCTTCTGGNNTNTTTATNNAACCGNGGACAATNNT
CANCNNCANCAATTCTTCTCNNANTNTNCCCNAAANTTANCNTNANGACNATAACANNNGNNCNATA
AATNATTTNAANCNCTTTTAATNNAATNCTACCNNCCCNNTCTTNAANATTTCATTATTANTNATGNCC
CATCNCAACANNTCTNTTTTATNTATTNNGAGCNCNANNGGANANNCTNTTTNNCANTTNNCATAACA
ACCNAANTATCTTNTNANTTAAACANNCANTNACANANNNCATNNTACTTANTNTANATANATNNAN
CCNNACAANNAANTNNTACTCNTTCNCTNNAANTTNNNTNANCCNTNTCNNCNATANAANNANATNA
NNTAANNNNCGCTGNGCNNNCACTNTATNTATNNATAATAAANAAACGCCTCNTNCNNGACNNTTNA
NTCNATTNANANACTNTAAAATTCTAACNATCNTNANGCNNGNTTNATCCCANTTAANTCACNCCNCC
CCANNTATAATTCNCNTTTNTNATTTGCACNGNTCNCTNAANNNNTCCTTAANNTNNACNNNNNATCC
AAANANCATCCNCNTATCNNCCTNTTAATNNTAANGATTTNNNTATNCNCTTNNCGNCTGTCNNTTCA
ACNNNNTCNATTNCTTTANNCCNNCCANNGCNCCCANNANGNATNNTNTNTTATNTCNTTCANNGNNC
TATCAATCNTNAANNAGTATAATTANTNTNTNACNNCTNCCNNACATGCCNCNTANTAANCNANTNT
NACCATCCATCTCCCCATNTGNNATTCTTCAAGCTNCCNANNNNGCCTCNANNANTCTANNNNCGANC
NATAGCNCCNTCGCTNTACNNTCCNCTTATCANTGCCTCNTACTNANNNGCCCTNNCNNNTNNGNTAA
NATNTCNGNCACTNCTNANAACNNNTCNTAACANGAGATCTCAANNATCAANCNNATGNATGCAGNT
CNTTANTCTGNTNGANACNCTNANANCNTCACCGATTATNNCGGNNTCNATCANTATATNAGCCAG
ANTNCACNCANNAACNCNNATANCGTTAANATAGCATNTCACTNATGACNATATCTCTTAGTNANN
NTGACTCTANCTATCTNTCTNNTTCACNGNTNCNTNNTNTCTNCNNNATNCGGACTCGANACTNAN
ACTCNNNNNCTCACGTNNACAATNNAGCCG
TGAGGCCNNNTAGTGNNGTCGTATTACNGGGGCGGCCGCTNTATAACTAGTGGATCCCCCGGGCTGCA
GGAAATAACTCTGAGNGGTTTGCCCTTAACTTCTNCAATGNATGANAGTGAAAAATNTAGTTGCTTAG
CCGCTCANNCGTGNTACAACTCTTCGGNGACACCATGANCTATANCCCAACAGGCTCCTCCGTCCATG
GGACTGAANAATCCTGNAGTTTANACCATGAAGAGGCACTNNGGGACAAGCTNNGTATCTNTGGNCT
GATNAAANGACCATGCGNAAGTGACTNTGGAACTGGNTCNGNCCAATNGCTTCCTGATGCTTGCCANN
NTCCNNCCTNNCTNGNTTGGATANTCTCNGNTGAGGAATCANCCCAGTNTTGCNCTCCTTGGGCTTGN
TACTNGCACNATGAGANATNAGACCCTANATTGGNTTGACCNNAGATCCGTNNGNAACACANNTNGA
CNTTGAAGAACNTGGGNCANCAGACCNAGCNNTTGANNTTCCANCTTGGCACCANTGNTTGGACNNGAA
NCCATGTGGACCAGCTGCCCCNAAGTCTTNNTCCTCACCTTAAATTCCTGCAACATAAAAANTGNTTC
GGTNCNNNNNCNACTTGACTNTTTGGNNNNCCCGTNTGTGTTATTCNANCAANTAAACTCCTTNNCAGA
ANTTACCTNTTNTGGCGGNGNNGGGNAAGTNGGGNNTAGTCTTNNNTTCATNGTTATTTTNCTTNCNTT
NNATTTTANTCNNTTTANCGANTNANATCCNCCNTTNTTNTTTTTNGGNTNTANAGANNNTACANTNTC
NTNNNNCGATTNTTCCNNCGNTTATTTNNTNNCCCNCTANNTNGGAANGNNCCCGNNCGTANATCCTC
TACNGTNCTTTNNNCTTTNTTNCTNTGNGTGNAANANCCCNCCNGAAATTNCTTTTTAGCCGGCACTCN
NNTTNACTCNACTTTTTANNNTCATNTNNAACNCNTCGGNNATANNNTGANATTNTCTCGCTGTNTNA
AACNTGTNTCTCTCTNACATTTNGTNGTTCNNNCG
TGAGGCCNNNTAGTGNNGTCGTATTACNGGGGCGGCCGCTNTATAACTAGTGGATCCCCCGGGCTGCA
GGAAATAACTCTGAGNGGTTTGCCCTTAACTTCTNCAATGNATGANAGTGAAAAATNTAGTTGCTTAG
CCGCTCANNCGTGNTACAACTCTTCGGNGACACCATGANCTATANCCCAACAGGCTCCTCCGTCCATG
GGACTGAANAATCCTGNAGTTTANACCATGAAGAGGCACTNNGGGACAAGCTNNGTATCTNTGGNCT
GATNAAANGACCATGCGNAAGTGACTNTGGAACTGGNTCNGNCCAATNGCTTCCTGATGCTTGCCANN
NTCCNCCTNNCTNGNTTGGATANTCTCNGNTGAGGAATCANCCCAGTNTTGCNCTCCTTGGGCTTGN
TACTNGCACNATGAGANATNAGACCCTANATTGGNTTGACCNNAGATCCGTNNGNAACAGANNTNGA
CNTTGAAGAACNTGGGNCANCAGACCNAGCNNTTGANNCCANCTTGGCACCANTGNTTGGACNNGAA
NCGATGTGGACCAGCTGCCCCNAAGTCTTNNTCCTCACCTTAAATTCCTGCAACATAAAAANTGNTTC
GGTNCNNNNCNACTTGACTNTTTGGNNCCCGTNTGTGTTATTCNANCAANTAAACTCCTTNNCAGA
ANTTACCTNTTNTGGCGGNGNNGGGNAAGTNGGGNNTAGTCTTNNNTTCATNGTTATTTTNCTTCNTT
NNATTTTANTCNNTTTANCGANTNANATCCNCCNTTNTTNTTTTTNGGNTNTANAGANNNTACANTNTC
NTNNNNCGATTNTTCCNNCGNTTATTTNNTNNCCCNCTANNTNGGAANGNNCCCGNNCGTANATCCTC
TACNCTNCTTTNNNCTTTNTTNCTNTGNGTGNAANANCCCNCCNGAAATTNCTTTTTAGCCGGCACTCN
NNTTNACTCNACTTTTTANNNTCATNTNNAACNCNTCGGNNATANNNTGANATTNTCTCGCTGTNTNA
AACNTGTNTCTCTCTNACATTNGTNGTTCNNNCG
TNNNNAAANNCTGGAGCTCCACCGCGGGTGGCGGCCGCTCTAGAACTAGTGGATCCCCCGGGCTGCA
GGAATTCGGCACGAGGATATTCGCCTCGTGGCGATAGTCCGCGCCGCCTTTGACCAGCGGATGAAGAC
GTGGCAGCGCTGGCAGGATGCTCAGACCACCCTGCAGAAGAAGCGGGAGGCTGAGGCTCGGCTGCTG
TGGGCCAACAAGCCGGACAAGCTGCAGCAGGCCAAGGATGAGATTGTAGAGTGGGAGTCTCGGGTGA
CTCAGTATGAAAGGGACTTTGAAAGGATTTCAACTGTGGTCCGAAAAGAAGTGATACNGGTTGAGAA
AGAGAAATNCANGGACTTCAGAAACCACGTGATCCAGTACCTCGAGACACTCCTGCACTCACAGCAA
CAGCTGGCTAAGTACTGGGAAGCCTTCCTTTCTGANGCAAANGCCATCTTCTAATGGACCGANGACAC
CAAAGCCACCTGCCTGACGCTGCCTTTTATACACTTGGCCTTCANCTTTGNCGGGCCCAGNGATGCATT
CTGGCTAACCTGGANTTAACCCNTTNCTTTCTGGCCCCACGAACGGGCTTTGCCCAAGTNTNTTTTAAC
CGGTACNTTACTTAACTTTCATATATATTTTTCTTACCTGAAAGGAANAANTTTTTTGGTTTTAAACCAA
AAATNCCTNTACTTAGGGGGGGGGNAATAANGGGGAAGGGGGGGGGGGNTTTGGGTTTTAAATTTTT
TTAAATCCCCAATGGTTTTTTTTTTTTAAGGAATGNGGGTTAGGNAACTNGGGGGANAAACCNTTTTTT
TNTTACNTCCCTGACCGGGGGCCCTTGAAAAAAAAATTTTTTAAANAAAAAAACCTTTTAAAAACNGG
GGGNAANNNNNNNNNNNNNNNNNNAAAACCTTNGGGGGGGGGGGCCCCGGGGCCCCCAAATTTNCC
CTTTTTANGGGGGGNNNNNNTNNNNNANNNNNNNNNNNNNNNNC
ANAAAACTGGACTCCACCGCGGTGGCGGGCGCTCTAGAACTAGTGGATCCCCCGGGCTGCAGGAATTC
GGCACGAGGGGCGTNGCTGCTCCTTGCGCTGTGCGCCGCAAGCGCCCGGGGGCTTTATTTCCACATCG
GCGAGACCGAAAAGCGCTGCTTCATCGAAGAAATCCCCGACGAGACCATGGTCATCGGGAACTACCG
CACCCAAATGTGGGATAAGCAGAAGGAGGTCTTCCTGCCCTCGACCCCCGGCCTGGGCATGCACGTGG
AGGTGAAGGACCCTGAAGGCAAGGTGGTGCTCTCCCGGCAGTACGGCTCAGAGGGCCGCTTCACCTTC
ACTTCCCACACTCCTGGTGACCATCAGATTTGCCTGCACTCCAACTCTACCAGGATGGCTCTCTTTGCT
GGCGGCAGACTGCGTGTGCACCTAGACATCCAGGTTGGGGAGCATACCAACAACTACCCTGAGATTGC
TGCCAAGGATAAGCTGACGGAGCTACAGCTCCGAGCCCGCCAGCTGCTTGATCAAGGTGGAGCAGAT
CCAGAAGGAGCANGGATTACCAAAGGGTTTCGNGAANAGCGCTTCCGTCTGATCAAGTGAGAGCNCC
AACCAGAGGGTCCTGNGGGGGGNCCATCGNTCAAACTGTCATNCTTNATCCTTCACTGGCATNTGGGC
ANATGCGTCACCTTAAANANCTTTNTTTGAAGGCCCAANAAAGCTGGGNGTAATGGTGCCTCACCACG
TGAATCCTTTTCTTGGCACCCTNANNTTANTTTGGGGACNTTTCCCCCNCCCAATATTNTTATTCCCCCT
GGGATTTNTGGANGGGNAAAAAAAAAGNGGAAAAAAAGGAATTTTTNNCCCCCCTTTGGTTTCCCTTG
GGCAACCNAAAACATTTTCAAANTCANCCCCCNTTGGTTAAACCCTTGGGNTTTTTAAAGGGAACCCA
GGGNAAACTTGGGTCCCAGGGNTTTTAAAAAAA
TNNNNNNCANCTGGAGCTCCACCGCGGGGGCGGCCGCTCTAGAACTAGTGGATCCCCCGGGCTGCAG
GAATTCGGCACGAGGAATCTTTATTTTTAAAGCAAAGATCTGAAAAGTTCCATAAAAGTAGTATCTTC
TAATCAATGATTCATTATTAGCAGATTATATTTACTCCAACTAATTATCCAATCAGTAGTATACTTTGA
TATATACACATCATACTGGTACCTTGTAGACATTTAAAATACCTTTGATTTCAACCATGCGTAAAAAAG
TAATAATATAACTCCACAAACGTTGCTCTGGAGTACAACGTACACCTCCTGTGTCAGACTGACACCAA
CCTTCCACTCACCTGTTGGTACATGTTCCCACCATGATCCACACCCTCCTCTCTGAGCCTTCCTTCTC
TTCTCATTGCTTGAGAAGTTCAGAGTTGTACTTCTCAAGTCTGTGGTAGTAGTTTTGTGGTAGGGCTTT
AATAGCATAGTAAAGATTAGGCAGGGTCAAAGTACTCGGTTATGCCTTCTGATTTTGAGAATACATTG
AGATTCTTTATGCCATCTCATTCGNGGCAGAACGGGGCCTCTCATAAATTTTTTGTTTTATTAAATTTAA
AGAAAACTACAGTAGAAAATGTTGGCAAGCCAATTATATAACCAGGGGTATTTATTACTGGGTAAAGG
GCTTTGGACAGTAAAACCATGGGTCTGGGTCTGGCACATGTGTTGGCANAANGGGGATAGCAACTATC
ANTTCGCAATTCTACCANTTTTTTNTTTNTCGGGGNTNTTTTTAAACCATAAAGTAAACTTGGCCCAAT
CAAGAAAAAATTNTTTTATGGAAAACCTGGTNCCTACCTNCCCCGNTGGGGAAAAAATAAATCCTTAN
GAAGGCCTCCTNNTNCAAAGGTTTTAACAAAAGCTTCCNTTTTTTTTTTTGCCTTTTTTTAAAAACN
AACAANACTGGAGCTCCACCGCGGGGGGCGGCCGCTCTAGAACTAGTGGATCCCCCGGGCTGCAGGA
TTCGGNACGAGGCCAGTTTTCAGTTGTGTACCAAGGATGATTGCTTCAGNGGNAATATTAGNCTTCN
NCTACCTANNCNCATANAANTTTNATNANANNGNAANTANACAATTTGANAAGGANNAATTGAATTA
GTTAATTNNNATNANAGNNTTNTTTCANCATNATGCATTGTNCANNNCATAATNTTTTNGTACTTATAA
TGATANTNTCATNATAATNGNTNAANTNNGNNANGATGCANAAGNGNGNTGAATTCNNTNNNCNAAN
GGANTNGTAGAGACNTTTNNNTANAATATNANNNTGNCATAANTNCTCNNNNNTANTNATNNACNNT
GNTANNTANTTGAATGGTTATTANTGGTGNCNTTTTNTTGTAAGNCAAATGNAAATAATNTTNTCATTT
GTTTCTGNTNTATTNTCTGTNANAGACTTNNTGTGTNNATATTAAANNACATNANATATNTTTTTTTTN
NTTTGCNCCCCCCCCCCNCCCTAATTGTTAACATATTNAGTNTNGATTTTNTNTNATNCTAACNCT
NTTANATTNTATANAATAAATAGNTNAAGNNANTNTNTTTTTTNNAGAGNTNNNGNCTNAAGANAGTT
TTGGNTCANTTGGGNNNAGATANTGNTATTNGNGANNNNGANTCACAGTCTTNGGATNNGTTATNAN
ATNTTTANANANATTCTTTATGTNATTAATANGANANTAGANAANTTTGATNCGTTAANTTTANTCGT
NNCACNATTNATTAGTGTNATNATATNNNATNTNAGTGNGNTATTANNTATNTNGAANTANTAGNAAA
TANTTGANNTNNGNNTACGANNTNATNANCACNTTTTATNNTGNTTGTAGACANTATGTGTATTTAAT
TAGGATNCNTGNATAATTATANGAGNCNAGTTNTTATNGAAGNTNATGNTNGTNTNTNANTTNCTNTA
TTATATNAANATTCTAATTTATCTNCNNATGTNTAGTATTANGANANGGTAGNAGTANGTGTTGAANN
G
GNGCNNCCGGNTTNNTTTNTNNNNNNNNNTTNTNNCNNNCCCNCCCCNTCCCNCTNGNTTNCACCTTN
TNCCCNTNGNGNNANNNNGNAATNNATANNTTNACCCTNANGANCGNATACAGNGGCGGCCGCTCTA
GAACTAGTGGATCCCCCGGGCTGCAGGNAATTGGGCACGAGGGTTTTCTCACCTGAAAGGACTANTGG
ATTTNCACTAANGTCTATAGACTAAAGGCCATCCTGATTTCTAANNTCCCCTTCNCAGTTACCAGNGGT
CTCTCTCTTTCTTGCNGTCCCTTTCTGCCTCCTGTCCCACCTGTATATGTGTTTAAGGNAGAAAGCTATG
TTTTGGTAAGGANAGTNNTTGNACTGTANCAGCTTAGCATGGACATTCTTCTNNCAGNCCCANGCACC
CACTACTAGTACAGCATGAANAAACNGCCTACCCAGAANGAGACNGTGGTTTGAGAGNGCGAAGGCCT
GGGGAATTTCCCCACACNCCAAAAGAANACTCTCATTCANCAGCNAGGGACAACNGNTATTGCNNTG
GNCTCACAGGCAGACTTTGGCTCGAAAATATTCTTCGNGGTGTTANAATTGCCACNGCATTTANNGAC
ANGTGAACNANGANATAAATCTGNAAGANTAATNTTGTCACCAATGNACTGTTNCCAGGNNCTCTTNA
NTNCANATGANCCNATTGNGATNGCCACTATNGGNTCACTAANNGCNCCNTTCCCNTTNACTTTGGNT
CAATTAATACCATTCNNCTANCNAANATTGGCNNACCAGGANCCACCANNANTAGGGAGTAANCCAN
NNTAGCAATTNTTTNCCCTTTGNCNCCGTGGNCAANTTTTTNGNGATTCNTAATNGGNNGGNTATTC
CCCCTGGGGGGNANNANTTGTTNCNAANNAANNNCNNNACTCTTAATAATACANNTNCTNCCCGTC
NTCTCTTTCTGNCNGANNGGANTCCNNTCNTNTCNNNANATCGGNATGGNNTTCNATNCCTTNTNCNC
ANCAACNNCTTNTTTTTCNANGCACTANCNGCNGNNCTCNNGGTATAATAAANAGCCTNTCTGNNGTC
NACNNAAAACCNCCNTNNANTNAGTCNTGTNANNANNANGNNGNGNCNATNTCNTTTATTTNTNAGN
CAGNACTTCGTGATCTCNNAGANCTCNCTCTTNCCCCTTTAGNGTACNCGCGTNCNNCATATCNCGA
TANACACANCNTGTCCTNCAGNGATAATTANGCNCTANTTTCGACGTTCTTCTANGNATACCNTANA
GTAACCNANTNTANATTCNCTCCTCTCGATCNCTTTTCANTCCC
GCCCTATANNGAGNTGNATNACAGTGGCGGCCGCTCTAGAACTAGTGGGATCCCCCGGGCCTGCNGG
AANTTCGGCCCGAGGGAGAAAGGTTCCTGAAGCTTTNGGNTTNTCGCGGCTGAGTTAGGGATTCAACT
TGCCAGCATGTCGANCAAANGAGCAAGGNCCAGGACGACCANAAAAGCGCCCCAAGNGCGGCNAACCT
TCNAACGGTGTTCCCCNTTGTTNNNCCATNCCNAAATTCAGNANTTNAAGGAGGCCTTAACNNTNANN
ANCCAAANCNGGGTNGGGTTNTTGNCAAGGAAAACTTGCNTNACNGNNCTNGNTTCCTTGGAAAAA
AATCCNNGTGNNNANTTTTTGNATCCCATANNAAAGANGCGTNCCAGGNCCCATAAATTTCCNNGTGT
TTTCACANATGTGTTGGGGAGAANTTTNATGGGGCACATCCCCAAANATGTGTCNGAAAAAGCTTTTG
CTTGNNNTTTTGAANAAAAAACCTTGTGGCNCTTNTTGGAGGGATNATCTCCTGANAACTCTNNNCCC
CAATGGNGGGACCCCGTTNNACATTATGAAAGTTGGGCCCACCTNTTTCACAAAAAACCCCTTTTTNT
NCGAAAAANGGGGGGGTTTTTTTTNCTTCCGNNNNTTCCCCCCNCTNNNTTTTTTATNGNGGGGANAC
GGAACNANNACCGACTTANTTNNNANTTTTTTTTTTCCTCCCAANCNTTCCTTTTTNTTGCNCCTTNG
GGGTNTTTTTTTGAAANATTNTTTTTTTTAATACCNGGNGNGGNTGCCCTTTANCCTTTNAANNNTTNN
GGCCTTTTTTTTTNNNTTTAAAANNNAANCCCCTATTNGGGNGNCANCCCNNTTTTTTNTTNTTAAAAA
CCGGAATTTNGGGGNCNTTCTTTTTTTTTTNAAAAAAAAAAAAAAANAGGTTTTTTTTTTTTTTTNNTTT
TNTGNGNNATTTNNNTNCCCCCCCCNNNTTACTTNGNNNNTNNNNNNNNNCACNTNTTTTTTTTTTNA
TNAANNGNNNNTNNNNCNTNTNNTTTTTTNCCCCTCTANTCNTTTTTTTNNCC
NCGTNAAGNGGCGNNTGNTCNTNNCCTACNCTCCTNTNCTGNTTAGCCCTATAGTGAGTCGTATTAC
AGGGGATTNCACATCCACNCNGGCCNNNTCCCCTCTNTCTTGCTCNCCNNTCTNCNNCCNTCNTTCGAT
TTTTTTAGTTTTCCTGCNTATCTNCTTCTNCTCNAAGNCNGATTCTTTCTCCTGCNCACCTANCTNCTTC
TCNNTTTNCGCNCTATNTTTCACTACCATTCCNCNNGCNCTCCNCGCNNGACCTCTCATNGTTTCTNTTT
CCNCTNTTTTTCCTNTTTNCNTACNGNCNGTNGNTCCANTCTCNCCTCTTTTANTNATATNNNGCCNCC
CGNTGTTNTTNNTCNCNNATNCACTNTCTNCCCCNCCTNCTNCNCTTTTTTTTCTNTCNATCNCANGNTC
CCNNCANCTTTCCNTCCTNTATCNTNCTNNNNATCCCTCNNNNNNTATNTACNTTNTTTNTTGCTCCCC
NNCCGGNCACACGTCNNTNNNTTTNCTTCTNCACTNGAACNCCNCCTCTCCCNNTCNCTCCCNTNACAT
CTNTTGNNNNNNTTCCTNGAANNCACTNACTCTNTCTTCTCTCCTNTCTATGANCCCNCCNCNGCNTTC
TTTTNTTNTCCNTATNTCTNNNCCCTNNTCNTACCCTCCTTCCTNTNTTGCTTCNTTCNCCNNNCCCCTC
TTTCNCTNCTNCNTCNNCTCCTNNNACNCACNTAATTCNGCCCCCNTTCNCGCCTCCNTCGTACTNNNT
CTTCATCGCTANNGTNTCCACNNTANGGGGCNGTTNTGNATNATCGTCGTTNATCNTTTNCCGCNTGNC
NCNTTCCTCTCNTTTNTTATCNTNTTNTNTCCTACACNCGCTTACGCCNNGCNCGCTCNNCTCNTNCTCC
TCTANTNGCTTCCGNTTCGCCCGCTCTTCGGCGNACCNCCTTCNGCCCTNCTNTCTTTATNTCTTAATCT
TATTCAANCNACGNTNNCCGTTNNTNTCNCCCTNNTTTTNTANTTCNTCANNCGNTCCGGCNCNCGCNC
TTTACTNTCTCTTNCCCCGCACNNANTCCTGCNTCCCGNCNATNTCATTTANTTGCNTNCATCCNNNCN
ACTNTNTGTCCTCTCNNTTCTNNCACCNNNGTATCCTTTCANANCNNCTCNCCNCGCTCTCNTGNTCNN
CNCTNGNTCTCTNCTACTNTNCTGNTCNTTCTCGNNTNTCTTNTACCTNTNTGCACTCCANTNTNGNAT
NTATACNTNCTATTGCCTNCTTGTACTNNTTTCTNNCCNCCTNNTNCCCGGANATCNACCTNTTCNTAT
CNTNACTCNCNNNATCTTCTNNACCNCNCNAANCTCTNNTNCNGTGTACTCGCATNTACCTNNNNCNC
CCNCATNCNTCNGGTTACNNTACTATGNGNCTCTCCATTTCCNNCTANGNNTCTNCTCNACTCTNTCCT
CNNNCGTCCG
TTGAGCCCNATAGNGAGTCGTATTACAGTGGCGGCCGCTCTAGAAGTAGTGGATCCCCCGGGCTGCAG
GCAANCAACACTGNATGGGTNCCCNTTNCCTTGCCNANTGTNTGACAGCGANGAATNATTGTNCTTAA
TNGGNCAGNGNGCCNNCAANNGCNNGNNNTCAACNTTAANTACAGGGNCANTANNACTTCAAAGNN
CCNTNGCANNCCAAAANCCGNGCACCTTATAACCCCGANGGAGTNACNNCCNNACNTGCNGTGGNCN
NATTAGCTCTGGANCCANGGTGAGNCCTNGGCCNAAGGAGGGCTTTAGATCNNNNGGGGACCAANTG
TGCNNGNGATCNCTGGGACACGGCTCGCACCCTTTACCTTAGANTGGAGNGTCTTATCATTTNANGNG
AGTATTGNNANATAGAAGCTCTNNTTGTGCCAGATCNANGGCACATATNCTNNTATTTATCCTCNAGA
NGNGNTGNCTCTTTNCNAATGNGGGGAACTNGAGNTNTACTCNGGNTACANTNGNACNTNAGNCCCA
CGTATNCGCACACCNNGGGNGAANAGAATNTTANGGACAGACANANTANACTTNTNTTTGGGNACNN
CNNGNCTGGNNTNANGNNNANTAAANANGATNGNTTGCTNAACTCNTACAAANTAANNAANGTNCTN
TNGGGGGACCNNNANGTNCNTTTNNCCAAANTGTCNNNNTGAGAAAANTCAANCTNTTCCCCCATAA
NNNCCANNTTTNCCCTCTNATNANACNCTTTTGGTNTTAANGGGANCCCCTCCNATCANNATGGGGNA
CCANCNGTNNTNGANCCNNTGGNCNCGNNNNCCCNTNNNNCANNAGACCAANCACNNNCNTNCATGG
CAAANNNGTTACATGNTNTNTTCNTANCTCTCTNACNTTNTCANTGGGNTCNTTACCNGTTTNNNNAN
CTCNTAGCACNTTNCATGTNTNTTCTANNANGNTANACACNANNAAANTNNNTTCNNTTNNAATTNNGN
GGANGCNTACANAATNCGGTTCCTANNTNNAAAANNNTCNATANAATTCTNACCNNCCANCTANNTA
TTNNTNCGAANTNTCTACGANTNANGANGNANCGACCG
TTGAGCCCNATAGNGAGTCGTATTACAGTGGCGGCCGCTCTAGAACTAGTGGATCCCCCGGGCTGCAG
GCAANCAACACTGNATGGGTNCCCNTTNCCTTGCCNANTGTNTGACAGCGANGAATNATTGTNCTTAA
TNGGNCAGNGNGCCNNCAANNGCNNGNNNTCAACNTTAANTACAGGGNCANTANNACTTCAAAGNN
CCNTNGCANNCCAAAANCCCNGCACCTTATAACCCCGANGGAGTNACNNCCNNACNTGCNGTGGNCN
NATTAGCTCTGGANCCANGGTGAGNCCTNGGCCNAAGGAGGGCTTTAGATCNNNNGGGGACCAANTG
TGCNNGNGATCNCTGGGACACGGCTCGCACCCTTTACCTTAGANTGGAGNGTCTTATCATTTNANGNG
AGTATTGNNANATAGAAGCTCTNNTTGTGCCAGATCNANGGCACATATNCTNNTATTTATCCTCNAGA
NGNGNTGNCTCTTTNCNAATGNGGGGAACTNGAGNTNTACTCNGGNTACANTNGNACNTNAGNCCCA
CGTANNCGCACACCNNGGGNGAANACAATNTTANGGACACACANANTANACTTNTNTTTGGGNACNN
CNNGNCTGGNNTNANGNNANTAAANANGATNGNTTGCTNAACTCNTACAAANTAANNAANGTNCTN
TNGGGGGACCNNNANGTNCNTTTNNCCAAANTGTCNNNNTGAGAAAANTCAANCTNTTCCCCCATAA
NNNCCANNTTTNCCCTCTNATNANACNCTTTTGGTNTTAANGGGANCCCCTCCNATCANNATGGGGNA
CCANCNGTNNTNGANCCNNTGGNCNCGNNNNCCCNTNNNNCANNAGACCAANCACNNNNCNTNCATGG
CAAANNNGTTACATGNTNTNTTCNTANCTCTCTNACNTTNTCANTGGGNTCNTTACCNGTTTNNNNAN
CTCNTAGCACNTTNCATGTNTNTTCTANNANGNTANACACNANNAAANTNNNTTCNNTTNAATTNNGN
GGANGCNTACANAATNCGGTTCCTANNTNNAAAANNNTCNATANAATTCTNACCNNCCANCTANNTA
TTNNTNCGAANTNTCTACGANTNANGANGNANCGACCG
NAAAAGCTGGAGCTCCACCGCGGTGGCGGCCGCTCTAGAACTAGTGGATCCCCCGGGCTGCAGGAATT
CGGCACGAGGCCCAAGTTTTTTTTTTTTTNAAATCCCAGGCTTTTTNGAATGGAAAACATTNGGGGGCT
TTTACTGANGGTTNTTAAAATANTCATNATAGNANCTGGATNNGTAANCNCTTTTTTTTCCTTTTAAGN
NCNTTCAANNCCCAAAAACNAANCTNGNCTNTCCGTTTTTTTTNAAAGNANAGNNGTGNTTNCCNGGN
NCCAAAACCAAANNGNTAATTTAANTTTNNTTTTAAAAACTTTNNAANTTACNANAAGNNANTGNAA
AAAANCCCTTTGNTTGGCCTTTAAAAANCTNCGNTTTTTTNAANCCCNCCNATTTTNAANGAANNNNN
NNCTGNANTAANAAAANCCCNAGGNNGNTNNGTTNCCAAAATTNNTTTTTNNCTGGNCGGGTTNTTTA
AANGGNAAACCGGGGAAAAAAGNCCGTTTNCCTGGTTNNNTGGGNCCCACTTNNTGCTAAAANNAAA
GNCCCTTGGGGNAATTAACGGGACTGGTTGGCATTTNGTGGGAGGGGAGGAAAAACNTTTTTTTGCCC
AANGAAAGGAAAGGCCCAAAAACCCCNTGGAAANTTTTAAANTTTTTCCNANGGCCCGGAANCAAAA
CCCNCCNTTTTAANCCCCCGNTTTTTTNCCCCTCAANGGGGGATTCTNAAATNTTTTTCNGGAAACCCC
CCTTTGGGGGGTTNCAAAAGGAAAGGGNNGGGGANNCCCCCCCNGGGGGTTTGGGGTCCCCCCCCCC
CCCTTTTNAAAAGGNCCCAAAGGAANCCCTTTTTTTTTNCCCCNGGGCCCTTTAAAAAAAAAAAAGGG
CCCCTTTNCCCCCCCCGGNNGGCCCCNTCAANGGGGNGGGGGGGGGGGGCCCCCCCAGGAAAAAAAA
AAAAGGGNTTTAAAAAAAANGGGCCTTTTTTTCCCCCCTNNGGGGGGNC
TTGANCACAATCTGGAGCTCCACCNCGGTGGCGGCCGCTCTAGAACTAGTGGATCCCCCGGGCTGCAG
GAATNCNNCACNAGGCCCANCGNNNCTGTNNNCCNNANGGCTGNGCCTTTGGGGAACTGGAANCNTT
NNTGGGCNTTNNACNNANGNGGNANTNACTCATATNCNANNNATGACTGGNANCGNCCANNNTTTNT
NACNNTTNNCNAAACATCTGNGCTCCTNTAACTCACCNNGGNGCNNCGGTTTNTTTTTGNATNNAAC
GNTGGAGAANNCGTANGCCNACCAANCATACNNTTNACCCANNNTTTTTGAAANNTATNCATNAACC
TTNTGCNTTANAAANNNTGACTTTTTGTGNACNTNANAATNACNACNTGCTGTNATGNCNNATATNT
TCAAACTATGGGCTATGNGANTANTTCAAGCNNCCCGGNGAATTCCTTCNTGAATGTGGTNNCCACCC
GCCTAAGANATGATGNAGGGGNANCNGGGNAAAAATNATCATANTCNGCAGNGTNAGAGAGGGCCC
NTTGGGTAAAATANTAATAAGAACCNTTGNTCCNTTATCCCCAGCCGACTNTNNTTTTNTTTGGAGCA
GAGAAAANAATNTTTTTTTANNGCANNCTGGTGGGGGGGGACCCCANNCCCCCCTTGGAAGGGAATG
TCATNNTTANACGNAANNNCNNCCCACTTNCCCCTTTTTTAAACCCCCNTTTGTTCTTTCCCTTNNAA
AGGNTNNGGNGANCCTTAAAGTTTTTGGGGGANCACCCTTTTCATTGGGGGCTTAAAAAAGGGAGGNTTT
GGGGTGTCCCAANCGNAAGGGCNCGGACNCCCCCCNCCTTTAAAACATTTNNAAAANGGACCCCGGT
TTTNTNCNNTGCCAAGTTAAACNTTAANNNTTTTTTGNTCCCNGGTTTTCCACAAAAAGGGGGGGGGG
GGNNTNACCNTCNNCNTGAAANAAAGGGTTTTTANAAAAGGNGTCTAATTTCCCCTTTCNNNG
TTGNAANAACAANCTGGAGCTCCACCGCGGTGGCGGCCGCTCTAGAACTAGTGGATCCCCCGGGCTGC
AGGCANCAACCTCTAGAANTNACCGTTCCTGCCNCANGCTTCCAACACAGCGNGGGTCTCTCCTACTG
CTGGAGGNCTCTGCCCTCCACATCCTCATTCTCATCCTGCTNTNCNTGGCCACTNTGGACAAGTNCTGG
TGGACTNTACCCGGCANGGNGTCNCTGANTCTNTGGTTCGACTGCNNNTGGANTAGTGACNNNCANAAC
GTNGTCCTTCNATAACTNCACANACATCGGTTNGNTGTACACAGTACACCTCCTGNNTCAGATTGNCC
NCATCCTNTNACTCNCNTGCTNNANCNTGTTCATNCCANAANTTTNNNNCNTTCCTCTANGAGNCNTA
NTTCNTTNCTCATGTGCTTTTGACAAGTTCAGAGCCNGTACTTCTGAAGTCTANNGGTANTACTTATCG
TAGGNCCATTTCTTTNNTAACATAATNCTNGATNAACCCGCCCCTTCAGGAGGGGGCANGGTTTANGN
TACTGGCTATCANCCATGACNCCGGGTGGNGACTTTNTCCNTNCGCCNCTGGACCANGGGGGNATNAT
NNGACNCTCNCNCCNATGNAGNAATTCNNGGAAGTTATGGAACTTGTNCCNTAACCTNTNGAATNNCC
NAANANCGAATTNCNAGGNCCNNTTTNTTTTACACCNCGGANANCCNCCCNACCCGTTTNCTTNNTTT
TNTTTNTTTTNAAAAACAAATANTTAAATTTGNCTTCNCCCCNTCCGAAAAAAAAAAAAAATANNATN
TTTTGGCNCGCCCCCCCCCCCCCNTCCGTTNCGTCTTNTTGNGGGTNTATTTNCCNANCANCNNCCANT
CNTCNTNAATTTCACTCNNAAATCTCTTTNTTTTTAANCNTTTTATTCNCCNTTNTTGTTCNGCTNTAAC
CANNANNTNAANTNTCTCTTTCTNNTNTCNNGNTTACTTATACTGAACATNCATCNTTGGGGGTNNCT
TTCTNTTCCNCCANACNT
NACAAAACTGGAGCTCCACCGCGGTGGCGGCCGCTCTAGAACTAGTGGATCCCCCGGGCTGCAGGAA
TTCGGCACGAGGCTCCTCTCCAGCCTCCACCATGTCCATCAGGGTGACCCAGAAGTCCTACAAGGTGT
CTACCTCTGCCCCCAGGTCCTTCAGCAGCCGCTCCTACACCAGTGGGCCCGGCTCCCGCATCAGCTCCT
CGGCCTTTTCCCGAGTGGGCAGCAGCAGCAGCTTTCGCGGCGGCCTGGGCACCGGCATGAGCATGGCT
GGAAGCTACGGTGGGGCCCCAGGTTTGGGGGGCATCACAGCTGTCACCGTGAACCAGAGCCTGCTGA
GCCCCCTCAAGCTGGAGGTGGATCCCAACATCCAGGCCGTCCGCACCCAGGAGAAGGAGCAGATCAA
GACCCTCAACAACAAATTTGCCTCCTTCATCGACAAGGTGCGGCACCTGGAGCAGCAGAACAAGGTTC
TGGAGACCAAATGGAACCTCCTGCAGCAGCAGAAGACTGCCCGGAGCAATATAGACAACATGTTTGA
GAGCTACATTAACAACCTCCGTCGGCAGGCTGGAAACTCTGGCCCANGAGAAGGTGAACTGGAAGTG
GAGCTTGGCAACATGCAGGGGCTGGTGGAGGACTTCAAGACGAAGTATGANGATGAAATCCAAAAGC
GCACAGACATTGGAGAATGAATTTTGTCATTATTCAAAGAAAGGATGTGGGATAAGCCTTACATTGA
ACCAAGGTAGAGCTTGGAGTCCCCGCCTGGAANGGCTTGACTTGATGAAAATCAACTTTNTACANGCA
ACTGGTATGAAAAAGGAAAATCCCGNGANAATGCAATTTTCAAAATTTTTGGAACCCGTTCCGGGGGN
CCGTGGGCCCTTGGGNAAAAANAAACCCGGAAACCTTGGGANCCCGGGATNGGGGAT
AAAGCTGGAGCTCCACCGCGGTGGCGGCCGCTCTAGAACTAGTGGATCCCCCGGGCTGCAGGAATTCG
GGACGAGGGGGAGTTCAGTCTTCGTTCTTCTGTGTTCGCTGCTGACACCACCACTAATTGACAATGCAG
ATCTTCGTGAAGACCTTGACCGGCAAGACCATCACCCTGGAGGTGGAGCGCAGTGACACCATCGAGAA
CGTCAAGGCCAAGATCCAAGACAAAGAGGGCATCCCCCCAGACCAGCAGAGGCTGATCTTTGCCGGG
AAACAGCTGGAAGATGGCCGCACCCTGTCTGACTACAACATCCAGAAAGAGTCCACTCTGCACTTGGT
CCTGCGCTTAAGGGGAGGTGTTTTAAGTTCTCCCTTTTAAGCTTTCGATAAGTTACATTGCACTTTTCTT
TCAATAAAGTTGTTGCATTCCAAAAAAAAAAAAAAAAACCNGGGGGGGGGNCCNGGCCCCNATTNCC
CCTTTATNGGGNGNCNNTANAANNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNC
GCCTATAGGGAGTTGNATTACAGTGGCGGCCGCTCTAGAACTAGTGGGAATCCCCCCGGGCTGAAGGA
ATNAAGCNTCNGCCTNTGCGTNGTCCTTCCGTTGTCCGCCAGCCGTCAATGCTCCCNTCCGCGTTGCTC
CGCCGNCCGGGTTNGGGGCCGCCTTNGTNNGGCCAGGGTCCCGCTTTTTACGCCAAAGGNTGGCCNGT
TGTTAAAGGGCCCCTTGCNGGNGGGNTCCCGGGAAAAAAAATGCCTTANCNTTTGCNTTTAACCCAACN
AGGGTGTTTTTTAAANAAGCGCCAAACTTCCGCNAGGGTGGANNNNGCCCACCACAAACNGGNCCNT
TTGGAATCCTTNAAACCNATGNGCCCNTTTTGGANGTCNTGNGGCNAGGCNATANTTGGGGCCCACCT
TGANGNNGGCCCCCACTNNACAAATACTTTNGAAGANTAGGGGNCNTNTGACGAAAAGAAAAATGGAA
GACTNCTNNGAANAAAAAANNTGNNATTGAAAGAAACTGACNTCTTTNGNANNNNNGANAGAATTNT
GGGGGGNAAAAAAAAAANACANTTNTTGGAAAAANGCCACAAGGCNGGNTCTTGTTGGGGGGGCAA
AAAAAACGAAGCCCNCTTGGGGGNTANAAAAAACAAAANCCCANNATGGGNCGCCCAANGGAGNTT
CTTTNANAAAAANNTNTTGGGAANTAGGGGGGNTTCCNTNCNAAANAACTTGGCCCAGGGTAANTAA
AAANCCNGGGCCNCGATNCTTGAGAAAANGGTTGGGCCTTGGNAAAAAAAAAAACCCAACCTTCCCA
ATGGGNCGCCNNCNNTACCNANNGGATTCCTTCCCCCCNNGAAANACTGTNCTNNTAAAATAANCC
CATTTNGGGGGNCCNTCACNNNNNTTTTTTTTTTTNNACACAAAAAAACTCTNTTTNGNGGACCCNTCC
CCCTTTTTNANTTAAAAAAAACANCNNCTAGAAAACANNACCTTTTTTTTNTATTCNNNTNNGTNGAG
TTNGANGNANAAATNTANNAACAACTTTTCCCCCNCNNAAAAAANAAAAANANCTCCCCNCNGGGGN
GGGGGGTNTTNTTTNNNNATAANANANNATTTTTTTTNCCNCNNNANNANCANTNNTANAAAAAANC
T
GGNNNANGGGNGCGCGNACGTCAGCCACCCNCNNNCCNTTNNAGCCCNATAGTGGANGTCCGTATTA
ACAAANNNATTACANNNNNCCCCCNGTNTNCCNCNCCCCGTCNCTGCCTCCCCTCCCTCCACCCCNCT
TTNNTCCNTNCGCCCNCCCCCNTCNTGNNNCCNCNCCCGGNGTCCCCNCCCCTCTNTCCCCTTCNTNCN
ANCNCTTCTCNGNACNNCACGGNCCCNCACNNGCCGCNTCNCTCCCCCCGGNNCCCCCCNGCNTGGC
CCNNTTNNNNTNGTNTNANCNTCNNNCCCNCCTCCCGCCNNTNTNNGTTCCCGNNNTNGCTCGCGGCC
CNNCCCCNNTNTATCTTCNCNTCNCNCCGGCTCNCCCCCCCCCCCCTCCANCNNTNGTCCGNANTCCGN
CGNCCCCNCCGTCTNATNCTCNTCTTNANCCTCGNACNNTCGCNNTTGGCCNNCGNNNTGGNCTTNNT
GNCCAGCNGGCTCNTATNANTNNCCCNNNCCGNCNGCCCGGCTCNTCCCGGCNNCCCCCTTCCCNCCC
GCNCCNCTTCNCGNCACCCCGCTCCNNNTGGTGNCTTNCTCNCGGGGGGGGGGGCCCCGGACTTTTCA
CCTTCCGGGGGGTCGCCTCGNCNGCNNANNNCNNGCNCTACTGNNNNCCCNCNACCCNGNGCGATCA
TGCANCTCNNCNNTCCAATNTCCGGNGCCCNCGANGCGNGCGNCNNCGCCTGGTGTGTNGCCNCCTCG
CGNGCCNTCTNACCTNCNANCTCNGGTTTCCTGTCNNCCNCNNNCGGCNNCTNTNGNCCCACNNNTAC
CTTCGGNGCNNCNNCCCCGCCCNTCCNCTCNNCCNTCTNCTTCAGTCCCGTTNCCNGCCGCCANAAGA
NGCNGTGAGCNCCTNCTCNCCTCTNCNNGTCNGTNNCCTTATGCTCNTTNNCCTNCCNTANCNNCCNC
GGCGNNGNNCNNCGACNANNACTNTTTTACANTNNCNGANTTTCCGNGAGGNANNNCTCACTNCCN
NTCCNNNTCTNCGCCGNGCTCTTNCCNNCGCNTATNCNCNGCNNTNTCCATGCTCCGCNCTACCCGCG
CGCTNCACCCATCCGNNGCGCNNCTCGCCGNCNNCCTCGTCGNCCNTCCCGNCGNCGCTTNTNTNTNC
CNCTNTCCNCTNCGCGACGTNCGNCTNNTGCNGTCCACGCTCTGACCGCTCTCTTACNNGTGCGCGCN
CANNGCGCACATNCACGGGTCCAGNGCCTAGTNCGNGCNTNCGCNCNTCTNCCGCGCNCGTCACNNA
CGGCGCNCNCTCTTANNCCTNCANCCGCTACGTTNGCGCCCTCTNCGTTCGCCNCTTACTCGTCCCG
TTTNAGCCTATANGGAGNNGNATNACAGTGGCGGCCGCTCTAGAACTAGTGGATCCCCCGGGCTGCAG
GAAANAAACACTGGAGTGGGTTGCCATTTCCTTCTCCAATGCATGAAAGTGTAAAAATAAAGTTGCTTA
TCCGTTCAGCCGNGTGCAACTNTNGNCNACNCCNTNANTTNTANCCCNCCAGGNTCNTCCGTCCNTGG
AANNNAAANTNCNNGCNGTTANTCCNNTNAAAAGGNCCCCNGNCCANNCTTNGANTNTGGTNNNNAN
NAAAGNACAAGGNGNANNNNNCNTTAANNTCNTTTNNACNANNGCNTCNNNNNNNTGGNANCNTCN
ACCTTNANTGGTTGGAATCNTTTAAGTTAAGNATTAANCCAANCTNGTTTTCTTNGGCNTGGAAACGG
CACATGAGAAATAAAACCCTTAATGNGGGGANACCAATCCATGTGTGGAACCACTGTGACATAGAAA
NTCTGGCACACACACCAGCATGANAACCCANTGNGGCANNNTGTNANACAAANNCTGTGCCCATGTG
CCCNCAGTCNCACCNNATGNNNATTNTGCACAAAAAAGGGNGTGTTNAACCCCNNTGATTTGGGGGG
NCTNGGTATACACCANTANNTCTTNNNACAAANNATATTTTGGTGGNGAAGNGNACAGGGGGTTTCTT
ATATAATGGTGNAATTTTTCGAAGAATTAATACCGNTGAATNGGNGAATAAAATNTTATGNGTTTTAT
TAAAAAAAAAAAAAAAAAAACNCCCGNGGANATATTTACCANCNTTTTTTTTTTTTCCCCCAAAAAGG
GGCCCCCCNCNNGNNTTTTNAAAAAANNTTTACCCTCNTTTTTTGGAAAAAAACNCNCCNAAAAAACC
CGTTTNGGNGGAATNTTNGGGGGGGTTAACCTTNGGNAAAAAAAAAAAAATTCNAAAAAAGNGNTTN
NNNGCGGGNAAAAAACCTTTTTCCAAAAAAGGNNGAAAN
TTTNAGGCTATANGGAGNNGNATNACAGTGGCGGCCGCTCTAGAACTAGTGGATCCCCCGGGCTGCAG
GAAANAAACACTGGAGTGGGTTGCCATTTCCTTCTCCAATGCATGAAAGTGAAAAATAAAGTTGCTTA
TCCGTTCAGCCGNGTCCAACTNTNGNCNACNCCNTNANTTNTANCCCNCCAGGNTCNTCCGTCCNTGG
AANNNAAANTNCNNGCNGTTANTCCNNTNAAAAGGNCCCCNGNCCANNCTTNGANTNTGGTNNNNAN
NAAAGNACAAGGNGNANNNNNCNTTAANNTCNTTTNNACNANNGCNTCNNNNNNNTGGNANCNTCN
ACCTTNANTGGTTGGAATCNTTTAAGTTAAGNATTAANCCAANCTNGTTTTCTTGGCNTGGAAACGG
CACATGAGAAATAAAAGCCTTAATGNGGGGANACCAATCCATGTGTGGAACCACTGTGACATAGAAA
NTCTGGCACAGACACCAGCATGANAACCCANTGNGGCANNTGTNANACAAANNCTGTGCCCATGTG
CCCNCAGTCNCACCNNATGNNNATTNTGCACAAAAAAGGGNGTGTTNAACCCCNNTGATTTGGGGGG
NCTNGGTATACACCANTANNTCTTNNNACAAANNATATTTTGGTGGNGAAGNGNACAGGGGGTTTCTT
ATATAATGGTGNAATTTTTCGAAGAATTAATACCGNTGAATNGGNGAATAAAATNTTATGNGTTTTAT
TAAAAAAAAAAAAAAAAAAACNCCCGNGGANATATTTACCANCNTTTTTTTTTTTTCCCCCAAAAAGG
GGCCCCCCNCNNGNNTTTTNAAAAAANNTTTACCCTCNTTTTTTGGAAAAAAACNCNCCNAAAAAACC
CGTTTNGGNGGAATNTTNGGGGGGGTTAACCTTNGGNAAAAAAAAAAAAATTCNAAAAAAGNGNTTN
NNNGCGGGNAAAAAACCTTTTTCCAAAAAAGGNNGAAAN
CAAAAGCTGGAGCTCCACCGCGGTGGCGGCCGCTCTAGAACTAGTGGATCCCCCGGGCTGCAGGAATT
CGGCACGAGGGCCAGACGAGCGAAGGCAGGAACCGTGGNCGTCGGTTTTTTCGTTGGCCCCGCNAAN
NCCCNTTTNNNGCAANNANGGNCACCGTCCNTAAANCGCGCAGGNTTTNTGTANTAAGGNGNGGAAG
GCTTCACCCTNNCAANGGGNCCCAGTTTANNAGGGCCAAAANTNCCTTGTTTNCCCAGGNAAGGAGG
CNTTTTGNTCGGAANCAAAGNGGTTNCGGNGGCCAACCCAANCCATTTTTCCGGAGAAAGGTTAAACC
CCCCAAAAGAATCGGCTTAAACTTGGATNCCTTGAGCCCCACTNGNAAATCCAAAAGGTGCTTGCCNT
TTAAAAGNGCCAGCNTTTTTGACTTTGAGGAAATAAAAAAANAAANGGCCCAGTGATTCNNNTCTTAG
CTTTTGGATTCNTTTTTTCTTNTGTTTTGAGAAAAAAATGGTTGAAGTNCCAAAAAANTGCCTGGATGG
GAATTAATACCCGCGATTTTTCTACTTAAAAAAAAAAAAAAAAAAAACTTGGGGGGGGGCCCGGGCC
CCAATTNCCCCTTTGGGGGGGNNNTTANNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNN
ANCAAANCTGGAGCTCCCGGCGGTGGCGGCCGCTCTAGAACTAGTGGATCCCCCGGGCTGCAGGTTTG
CCNCTNATGGNTNATGCTTGNTGTNCNCANCACTCTNTCNCTCTGTCTANGGCNNNGAGCCNNCNGCNG
NCGNAANGCCACNATGTCCATCCTGAAGGANCACGCNNGATANANTTNTNNCTCTCGTGGGAATCCC
ACCGACGNGGTTGATCTCTNCACACGCTAAAGGTCAATGCNTAGNTGNTGTGCCCAANGGATGCTTCA
TCTTGGAANGTATGANGCCCTGGGGCTCCGGAACANTGATAAGACTCTCTNCATGGGAAAAGGTGTNT
AAAGGGNTGACGACCTCATCANNAAAANAATNGCCCCTGCCTTGNNTANCAAAAAGNTNAACCNNAT
GANGCAGGANAAAATCCCAACCTTANNATAAACTGGATGGCACANAAAATATTNTTGGGGTGCNANC
GCNATCCTGNCGTGCCCTGGNTATTTTAANGGCTGCAGCTNCGAAAAAGGGGGGCCCCTTTACCNCCC
ATCCCCCACTTTNNTTGCAATGCTTAACGNCATCCTGCCAATTCCGGGTTTTNAATGGCCTTCACCTTA
GGNCTNTNANGCTTGNAACCANATTGGGCCNTGCAAGGGGTTTATGNAGCCTTTCCTGTTGGGGGACN
AAANANTTTCCNGCNAGGGCCTTGCCCCACCCGGAACCTNAAGTTTTTAACCAAAAGCCTGGGAAAAA
GGCCNTTNATNGNGGNAAAAAGGGGGGGANGGGCTTTCCCACCAAACGGGGGGNAAAAANNANNNC
NGGTTTTTTGNCCCCNAAAATTTCNGGGNAAAAACCNNAAAAANCCCCCTGGAACCNTNNTTGNAAA
AAANTGGCCCNCCCNNAAAAAGGGNGGGGTTTCCCCTGGCCCCAAGGGCCCCCAAAAAGGGGGNAAN
GGGGGGNAAAANGTGGAGCTTTTGGANNNNTTCAANGGGCCCGNGGNCAAANAANGGAACCCN
TNNNNNAAAAGCTGGAGCTGCACCGCGGTGGCGGCCGCTCTAGAACTAGTGGATCCCCCGGGCTGCA
GGAATTCGGCACGAGGGTTTGGAGTTTGCTGTGTCTGTATTCANTAAGCAGATACTATTTATTTAGGTT
GGTGTAAAGGTAATTACAGTTTTGCATTGTTGAATTTTGCCATTTGATATTGAAATAGATTCTTAAATA
AATGAAGTTATGTTTTGAAAGTGCAAGTGTTAGTCACTCAGTTGTGTCTGACTCTTTGTGATCGCATGG
GCTGTAGCCTGCCAGGCTCCTCTGTCCATTGGATTATCCAGGAAGAATACTGGAGTGGGTTGCCATTCC
CTTCCCCAGGGGATCTTCCTGACCCAGGGATTGAACCCATATCTCCTGCTTTGCAGGCAGATTCTTTAC
TGTCTGAGCCACCAGGGAAGCCCGAGGTTACGTTTTACATCATTTTAATGCACATTTCTCACTTTATGT
TTTTTTGNTAATGATNTATTACTTGCTGNTTATTTTAAACTAGGGAAATGATGTTAGACAAAAAGCAGA
ATTGAACGATTTTCTTATTTGAGTTCAAAATAAGNCNTAANTCANCAGAAACAACTANCCACATGAGT
GACNCTTTTGACCCAGGACTGNTAACAAACATCCGGGCANTAACCCGTTCAGGAAGGCTTTTTNAANGA
AACGAANAGCTTTTACAATGGAGGGNGGCAGAAACCTGGCCCNTNAAAAATTGGNCAGGGACCTAACT
GGGGAACCCNCCCGGNGGGACTTGAACCNTTTACCAGNTTCCCTNGNNAAAGNNGCCCAAAAAAACT
CGGGGGCNANCCCCNCTTCCNAGTGGNTCNNCCNNTTTNAANCCAAATTTTTTANGGGGNNAAAAAN
NCCNAAAAACCGGGGGGCCCNCTTTGAGACCCCCCCCGCNAAAAAAAAACCTTTTTTTTTNNAAGGGG
ATCC
TTNNACAAANCTGGAGCTCCCCGCGGTGGCGGCCGCTCTAGAACTAGTGGATCCCCCGGGCTGCAGGA
ATTCGGCACGAGGCACCAGCCCAGGCTTGGACAATGGGTGGGGACATGTTCAACGCCAGGTTCATTCG
AAACTTGCATGAACGTCGCAGCACCAGGCCTTGGTGACCGCAGCCCCCTCTCACACCTTCAAAATCAT
TATTATTTCTATCTCACACAGGACTTAGTCAAAGCTTGCCCTCGCCGTGTATGTATTCATTGATTGATTT
ATTCATTCTACAGGCTTTTTTTTTTAAAATCAGGGCCAAGTCATTNGTATCTTATTTTTCCAGAGCAA
TAAAGTATTTTTTACCACGAGAAAAAAAAAAAAAAAAACNTGGGGGGGGGNCCNGGNCCCNANTTCC
CCNTNNNGGGNGNNGTTTNANANNNNNNNNNNNNNNNNNNACCTGGAGCACANACAAGGNTCTGGA
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNCNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNN
GCCNTATNTGGAGCTNNATNNCNGTGGCGGCCGCTCTAGAACTAGTGGATCCCCCGGGCTGCAGGCTT
CTCTGGTCTCCNGGTCCCCCCGGCCCTCCCGGCTCTCCTGGTGAGCAAGGTCCTTCCGGAGCCTCTGGT
CCTGCTGGTCCCCGCGGTCCCCCTGGCTCTGCTGGTTCTCCCGGCAAAGATGGACTCAATGGTCTCCCA
GGCCCCATCGGTCCCCGTGGGCCTCGAGGTCGCACTGGTGATGCTGGTCCTGCTGGTCCTCCCGGCCCT
CCTGGACCCCCTGGTCCCCCAGGTCCTCCCAGCGGCGGCTACGACTTGAGCTTCCTGCCCCAGCCACCT
CAAGAGAAGGCTCACGATGGTGGCCGCTACTACCGGGCTGATGATGCCCAATGTGGTCCGTGACCGTGA
CCTCGAGGTGGACACCACCCTCAAGAGCCTGAGCCAGCAGATCGAGAACATCCGGAGCCCTGAAGGC
AGCCCGCAAGAACCCCGCCCGCACCTGCCGTGACCTCAAGATGTGCCACTGTGACTGGAAGAGCGGA
GAATACTGGATTGACCCCCAACCAAGGCTGCAACCTGGATGCCATTAAGGTCTTCTGCAACATGGGAAA
CCGGTGAGACCTGTGTATACCCCACTCAGCCCAGCGTGGCCCAAGAAGAACTGGTATATCAGCAAGAA
CCCCAAGGAAAAAGAGGCACGTCTGGGACCGGCGNAGACATGACCCGGCGGATTCANTTTCGAATAT
GGCGGGCAGGGGGTCCGATCCTTGGCGAATGTGGCCATTCAACNTGANCTTTTCTTGCGCCTTGAATG
TTCAACCGAAGGCCTCCCCAAAAAATTTCACCCTACCACTTGNAAGNAACAAAGNNNGGCCCTAAATT
GGACCAACAANACTNGGCAANCTTTAAANAAGGGCCTNGTTTCTTCCAGGGGTTCCAACGNAAANTC
AAAAACCGGGCCGAGGGCAANAAGCCCGNTTTTAACCTTAAGGGTTAACCTAANNANGGGTTGACNN
AATCNNT
TTTAAGNCCTATAGTGNNGTCGTATTACAGNGGCGGCCCGCTCTAGAACTAGNGGGATCCCCCGGCTT
GCAGNAATTNGGNACGAGGCTTTTTTTCCGCCTGGNTATCCGAATGGGCCCTGCTTTATATCACACTNT
GCNTAGGGGTTCCNGATGACNTGGCAAACCCCCCTTGTTTNTGGGGCCNTGATNACATNAAGANTTTN
NTNGNCAAAANCCTTGNNTTNAGNNANNTTGGACCGGGACACAAANAGGNGCNNTTNGNANNCGGG
NGNGTNNTTCCCCNACNGGCNTATAGGNNNNGTGCCATCANNTTNNCCCTATATTNCACCTTANCNCT
NNGCTCTNNAAAANAANATGTNCNNGGGGTCTANATTTTTNTNAAAAGGGGCACTTNTACCCCTCTCA
CTTTTGNCTTTTTACACCAGNGNGAANNGNANGAACCTCTTCCCCCNTCTNAANAAAGNTGGNCNTNT
NTCNTNTNNTNCTTGTGCCNAAGGNGGGGAAANANGNTTTATTTCNNNGGNCCNCCAAAAANCCTTNA
AAAAAANGNGGGGGNCTNTTCTNTTTNNANNGTNTTNCGGNGNGAGAAACNTATGTCCCCTAGAANT
ATTACTNGANANGGANNTNCTNNNANTCAGCTCNTNCAANNCTNGTTAANANNGTGGNNAGAAANTA
NANATNCCCTAGGGNCNTANCCANGNGTATGNGATNCANNCTTGNCTNGACTNTCCCNCANTNNACG
GGANNCTNAGAAANNANAANCTNTCGTGNCCCTNGCTCGCGCNNTATTANGNNNTCTANNNNCGGAG
CCCTTCCCNCNTNTTTNTCNAGNCGTTCCNCTTNNTAACTNNACNAAACTTTGCTTCNNNANCCCC
ACCTNTTNTTTTNTNTTTTATGNCACNCTNNNNGCNATTGNNCTCCACTNGACNTCGNGGAGNCNNNT
CNNCCCTCCCTTNCNCNNTNTGACTANCCCAGTCNTGAANACTNGCCGCANNCNNCTTTNNTTNNNAG
ATTACCNNTNGNNGCNNTGCCTCNCTCCNTATTATNCNTANGNANTNGNNCCNCTAANGACTCCNCN
TANNACANCATNGAGTACGNCATTTTNAATCTCTTCGGATNNCNANTACGNNTATCTTNGTTNCNNCC
NNTCTNGGGCNNACTCNGCCC
TGCNNGNGNNNNNGCCANNTCNCTCNNNNNNNNTTNNAGNCCNATAGTGGAGGTCGTATTACAAANG
GANTACAGTNCNAAANCGGNGGGGTACCCCCNGANTTGCNCTNATNTTTCTNCTTTTNTNCTTCTNTTT
CNCNTNTCNTACGCNTCCTNCNGTGGNTNCTCNGGGGNGCCTNTNCTTCTCNNNCNNCNNTCACTCCC
CNNCCTTNNNCNNACCTCTTTNCCTTGNCGNTNCATTNTCCCCTGTTNGTNTTCATCATTGNGTCTNCT
NNCTCCNGNNNGTNNGCNAGCGNNCCGTNNCNTNNCCCCTCTNTNTNATNGTTGGNCTCGGCNCGNCN
GCCNTTGTNTTCTTTNATNNTCCTGCNCCCNATGNNTGCNTTNNTTNNTTNTTNNTATTACCGCGNNCC
NCNCCNTNTTNTTTTANTNTTTGTNTCCNCNCCACCGCTNNCTTATATCNNTNNNTGNTCGGCTTTCNN
NAGNCATNCNATNNNTCTNTCTNCNATNTTCTNNCNTTACTCTAGCNCNNGNNTTCNCNTCTTTTCCTT
GCNCCCCTNCCCGTCNNGTNNTTCTTTTTNTTNNGNNGCGNCTNCCGTNCTNTCCCTNNCTNGNAANTT
TCTNGCCATNNGNNNANNCNTTTTCNTNGNNNNTTGCNNNTNNANNCTTNNNNCTTNTNTGNNCTCTA
NGCGGTCNNANANNNGCCNGCNNTCTGTNGCATTTACCCTTTGNTNNGGCCCNGCNNNGNNCNTNCTC
NTGNGNNTNCNNNNNCNGGNNNNGNNGCNCNNTTNNNNNNGGTTTGTGGGNNTCCGNNNCNCTNTGT
TTTTCNNGTNNCGNCCNTCTTCTATCGNNNGNCGTNNATATGNCCNTTNNNTCATTTCNTACCNCCNNN
CNNGNCTCNCNTCNNNCTGGTTTTNTCTTNCCCTCCNCCNCTCCCTTCACCNTCTATTTCTANCNCTTTT
CCNGTTACCNCGCTTCGNNCTGCANGCAACTCATNNTCNGCNTTATNCTGCTNATTTTCNNCAAGCANT
NATCNGNGNTCTCCCNGGNTTTTNCACNCTTNNTTNCTCNTATTNCCCCNCTNNNTTCCTCATNNNTC
TNGCATCNTTTGNTNNCTANTNATGGCCANTNTTANNTNTANTATTTTTTANTGCCTCANNGTATTTNC
TCACATATTTCTNCTTTTCTGTTTNTCTTCGCGCTNGCNNTTAGATCAGCTNCTNNTTNTCCTCNATNNA
TNNTTATCNCGTCNTTCTNNNTCATNNCTCTNGNNTCGNCNACATCTCACCTNTNGCGCTCTCGTCNCG
CGNNCNCNCCC
TTNAGCCTATNTGGAGCTNCANCNCNGTGGCGGCCGCTCTAGAACTAGTGGATCCCCCGGCGTGNAGG
AATTCGGCACGAGGCTCCCCCCTCGCCCCGAGCGCCGCTCTGGCCCGCACTGCGCTCGCCCTGAGCTCC
GGCCTCNTGTTAANCCAGCNCCGTTGNCNCNTCCTTCNAGNCNCNNTNNTNANNNTTNNCCGGANCNT
NNTTACCCTTNACAAAANGTNNTCCNNCTTTNNCAAAATCCGGANGGNCNNGNCCGGCNTGNNNTNG
AAGGGGNAGGGAAAAANGGNNATTAGNNCAAAGGNNACNNTNANNANNTNCTTNNTNGTGGNAANN
NCNTCNGTNAAGGCCCNAAGGCCAANNGGCNCCAAAGNCACANNATTCTGTGGTGTNATATTTGATT
AGACCCATCNNTTGCNGGAAACCAGNTNCACAAAANACNTCNACAANATGCATTCAGANNTCANGTG
ATNTCTCTCAGGGGNACTNTTGGAANACAGACCCANAGAGNGTAACCCTTTNTGNNACGGGNNGTGN
AANANANAAANCNCATACTTTNTTATTTNAAAAACTAATATNNTNTTTNTTTGTGGAAAACANGAANN
NNCANGGGGGTGGGNGGTNCTNTGGACNCNACCGAGGAANGGNGTGCCCCNTATATATNATTTTTTAT
GANGGNGGTTTAGAAGGTGGAAATNTGTCNAANTGNGNGGTTCTTTTGATNANANACCTGTGNCATA
ANAGGGNGGGGNCTGCTNTTTTTTCCCCCACCANCACGANTTNTTAAAAAAGGGNCTGATGGTGTCTT
CNGCCTCTTTTTTTTTTTTTTACAACCGTTTTNTTTTTNGGGGGNGGCTTTTTTTTTTTNAAAAANANCTC
NTTTNTGGTTGGTTAAAAAAAAAAAAACCCCTNTTTTACCTTTTTGAGGAAA
TTNAGCCTATNTGGAGCTNCANCNCNGTGGCGGCCGCTCTAGAACTAGTGGATCCCCCGGCCTGNAGG
AATTCGGCACGAGGCTCCCCCCTCCCCCCGAGCGCCGCTCTGGCCCGCACTGCGCTCGCCCTGAGCTCC
GGCCTCNTGTTAANCCAGCNCCGTTGNGNCNTCCTTCNAGNCNCNNTNNTNANNNTTNNCCGGANCNT
NNTTACCCTTNACAAAANGTNNTCCNNCTTTNNCAAAATCCGGANGGNCNNGNCCGGCNTGNNNTNG
AAGGGGNAGGGAAAAANGGNNATTAGNNCAAAGGNNACNNTNANNANNTNCTTNNTNGTGGNAANN
NCNTCNGTTNAAGGCCCNAAGGCCAANNGGCNCCAAAGNCACANNATTCTGTGGTGTNATATTTGATT
AGACCCATCNNTGCNGGAAACCAGNTNCACAAAANACNTCNACAANATGCATTCAGANNTCANGTG
ATNTCTCTCAGGGGNACTNTTGGAANACAGACCCANAGAGNGTAACCCTTTNTGNNACGGGNNGTGN
AANANANAAANCNCATACTTTNTTATTTNAAAAACTAATATNNTNTTTNTTTGTGGAAAACANGAANN
NNCANGGGGGTGGGNGGTNCTNTGGACNCNACCGAGGAANGGNGTGCCCCNTATATATNATTTTTTAT
GANGGNGGTTTAGAAGGTGGAAATNTGTCNAANTGNGNGGTTCTTTTGATNANANACCTGTGNCATA
ANAGGGNGGGGNCTGCTNTTTTTTCCCCCACCANCAGGANTTNTTAAAAAAGGGNCTGATGGTGTCTT
CNGCCTCTTTTTTTTTTTTTTACAACCGTTTNTTTTTNGGGGGNGGCTTTTTTTTTTTNAAAAANANCTC
NTTTNTGGTTGGTTAAAAAAAAAAAAACCCCTNTTTTACCTTTTTGAGGAAA
CAAAAGCTGGAGCTCCACCGCGGTGGCGGCCGCTCTAGAACTAGTGGATCCCCCGGGCTGCAGGAATT
CGGCACGAGGGCGAGACGAGCGAGGCAGGAACGTGGACGTCGGGTCTNTCGTTTGCACCGCGAAGAC
ACCTCTNCTGCAAAGATGGTCAACGTNCCTAAANCCCGCAGGANTTTNTGTAANAAGNGTGGAANGCT
TCANCCTCNCAAAGTGNCCCAGTTTANGANGGGCAAANATTCCNTGTTTNCCCAGGNAAAGAGGCGCT
TTGATCGGAAGCAAAGTGGNTNCGGGGGCCAANCCAAGCCATTTTTCCGGAANAAGGCTAAANCCCC
CAANAAAATCGGGCTGNAACTTGAATGCGTTGGNCCCAACTTGCAAATCCAAAAGGATGCTGGCCNTT
AAAAGGGGCAAGCTTTTTGAACTTGGAGGAAATAAAAANANAAAGGGCCAAGGGATCCANTTNTAAG
CTTNGGGATTCTTTTGGNCTTAAGGTTTGANAAAAAAATGTTGAAGTTNCCNAAAAATTGCCTGAATG
ANAATAAATNCCGNGATNTTCCTTCTTAAAAAAAAAAAAAAAAAAAAAACTTNGGGGGGGGGCCCGG
GCCCCCAATTTCCCCTTTAGGGGGGNNNNTNAAANNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNN
GGGGTTNAAAAAGCTGGACTCCCCGCGGTGGCGGCCGCTCTAGAACTAGTGGATCCCCCGGGCTGCAG
GCGCGCCCTGAGCTGCAGACCGACCTAGAGCCCCCGCCCCTGCCCGACTGCTCACCCGCCGCCGCCAT
GCCCTTCTCCAATAGCCACAACACGCTGAAGCTGCGCTTCCCGGCCGAAGATGAGTTCCCCGACCTCA
GCGGCCACAACAACGACATGGCCAAGGTGCTGACCCCCGAGCTGTACGCCGAGCTGCGCGCTAAGAG
CACTCCGAGCGGCTTCACGGTGGACGACGTCATCCAGACCGGCGTGGACAACCCAGGCCACCCCTATA
TCATGACCGTGGGCTGTGTGGCCGGCGATGAGGAGTCGTACGACGTGTTCAAAGAGCTCTTTGACCCC
ATCATCGAGGACCGGCACGGAGGCTATAAGCCCACCGACGAGCACAAGACCGACCTCAACCCCGACA
ACCTGCAGGGCGGCGAGGACCTGGATCCCAACTACGTGCTGAGCTCGCGGGTGCGCACGGGCCGNAA
CATCCGCGGGNTTTTTGCCTCCCCCCGCATTGAAGCCGNGGGGAAGCGCGGGGCCATCGANAAGCTCG
CCGTCNAAGCCCTGTCGAGCCTTGGACGGNGAACCTGGGNGGGGANGTACTATGGCGCTTCTCAAAAGC
CTTGACGGAGGGNGGAGCAACAAGCAAGNTTATCGACNACCACTTTCNTTTTTGAANAAANCCCGNGT
CGGCCCCTTGGTGGTTGGNTTTTGGGCATTGGGCCCCGGGAANTGGGCCCGAACNCCCCGNGGGGATT
TTNGGNATTNAACGAAAAAATAAAAACCCTTTCTTGGGGGGGGGATNNAACCAANGGANGGGACCAT
TTTTGGGGGGGTTATTTTTCCATTGCNAAAANGGGGGGGCNAAAATTGNAANGGNGGGNGGTTCAAA
CNCCTTTTTGGGAAAGGGGCCTTAACCCNAAAATGGNAAAACCCNTTTT
AAAAGCTGGAGCTCCACCGCGGTGGCGGCCGCTCTAGAACTAGTGGATGCCCCGGGCTGCAGGANTN
CNNCCCTNGGNGACAAACCCGATNTGGCTGANATTGAGANGNNCGATAAGTCNAAATTGAAGAANAC
GGANACGCAAGAGAAAAATCCACTGCCTTCGAAACGAAACGATTGANCAGGAGAAGCAAGCAGGCGA
GTCGTAATGAAGCGTGCGCCGCCAGTATGCACTGTACATTCCACAAGCATTGCCTTCTTATTTTACTTC
TTTTAGCTGNCNAACTTTGTAAGATGCAAAGAGGTTGGATCGAGTTTAAATGACTGTGGTACCCCTTTT
CACATCAAAGANTGGAGAACTAGTGACANCGTAGGCCGCGCNTGCCTCTGCCATCTGCTTGTGTGGCT
GGCAGGGAAGGAAAAGAACTTGCATGTTGGTGAAGGAGGANNCTGGNTGNTACNACAGNGAAATCTA
CAGTAAAAAGCTGGTCCAAGGTGTTCTGCGGNCTGTAAAATGCAGTTTAATCAGAGTGCCATTTTTTTT
TGTTGTTCAAATGATTTTANTTNTTGGAATGCACATTTTTTTTNTTNTGCAAATAAAAAGTTTTAAAACC
TGAAAAAAAAAAAAAAAAAAAAATTTGGGGGGGGGGCNGGGNCCCNATTCCCCCTTNTAGGGGGGN
NNNNNNNNANNNNNNANNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNC
TTNNACAAAACTGGAGCTCCCCGCGGTGGGGGCCGGTCTAGAACTAGTGGATCCCCCGGGCTGCAGGA
ATTCGGCACGAGGCCGAGGTCGCACGCGCGAGGCTGCTCTGCCGCCGCTGAGATGCGTTACGTTGCCT
CCTACTTGTTGGCCGCCCTCGGGGGCAACTCCTCCCCCAGCGCCAAGGACATGAAAAAGATCCTGGAC
AGCGTGGGCATCGAGGCAGACGACGATCGGCTCAACAAGGTCATCAGTGAGCTCCACGGAAAGAACA
TCGAGGACGTCATTGCTCAGGGTATCGGCAAGCTGGCCAGTGTGCCGGCTGGTGGGGCTGTGGCCGTC
TCCGCTGCCCCAGGATCGGCAGCACCCGCTGCGGGTTCTGCTCCAGCCGCAGCAGAGGAGAAGAAGG
AGGAGAAGAAGGAAGAGTCGGAGGAGTCAGATGATGAGATGGGGTTCGGCTTGTTTGACTAGAGTCC
CGCTCCCCTGCAAATAAAACCCTTTTTATGTAAAAAAAAAAAAAAAAAAAAAANTNGGGGGGGGGCC
CGGNNCCCAATTCNCCCTATGGGGNGTCGNTTNNCANNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNN
NNGGNTNANNAATCNTNCATNCANNNNNTTTNTTNTTNCCNCCCCCNCCACCNTCTANCTCCATNC
CCCCCCNCCCCCCCCNCAANGGGGGGNACNCGTATGTTCCCCCNCTTTNAGCCNCATTACGAACNNN
CTCTCNCCNTCTCCATTNCTCACNAATACCTTGTTTNNNTCTTNCTTTTAACCNTTCCNNCCTCATCTCT
TNTTTCATNTTCATTCATCATATCANTNNNTTCNCCNATGAANTGTCACTCTACTCTNCNCACTTNTAA
CTTTTTNAACTNNNNATANCTNNNTANGNTNGCTNANGCCTNANCTNAATGATACNCAANGNTTNAAN
NANGTCTANNCACNNTNNGGGATANGNNNTGANAGNGNGTTACCNGNNNNGGGGANANNNNTTNGN
NANCTTANTANANCTAGTNTTACTTNCTAATCTCNNCTNNAATCNNACTTGCACGNANCCCTNGNAGC
TANNNCNGNNNNNANTGTNAGANANGTGGNNNNGNGNNCANNAGNNNNNACNNAACNATANANNG
ACNNNGNNNGAATNNANTTTGNNTNNAANATNNANTNCNNNNAAGTTNNTTNCCNAATCNNCGCAAC
TANGNNAGGGNCAANNACTTACNTANCTTCCCCGTTTNANANGAAANNNTTNGNNATGCNNCGNCTA
TNNANNCNATNNGNGANTCANNNNNNTAANTNANNCNNGGNCNNNNNCCANNGCTTNGTCNANTAN
ATTATNCTNNNANGCNNANTGANCCGNNAACNGACGANTTGACNGGGNGGANGANNANGGNNGGCN
NNNNGGNTANATATGCANTNGCNACNNAAGCTNCGNGANNNCCANNTTNTTANANAGTNTTNGTGNA
NNTCANGNTNGGNNNCACTCNAANTANNACGTGTGCGNNCNTGNCTGCAAGGACNTCNTAGNATTAA
NGNNNGNGTGACNAACACNNTNATGTGANCANAGCTNTACNNTNAGTTTCNTNTGNANACNNTGCTN
NCTGCGANATNGAGTNCGNCNNGTCNATANTNGTCNNNCNCNATNCCTNNTNNCTGNTNCTNTNNAN
CTCATNNNNAGTANTTNNCCTCANNCNCNTNAACNTGAAATATNCNNCNGTNGCTCCTTCANCATGTC
CNTGANTGANCNCTATGNNGNCNANNCAGNNTCNNCGANNATGTNCTCNNCGANCAGCATNANTACN
TTNANNACNTAAGNTATATNTCACNTANNNNATCGNCAATGTGCCNTANTAGAGNNGCGTANNTNNG
AGTCNTANNTNTACACATANNNTANNNNNTTGGCANNACACGATCCTGTTANTAGTAANNNCTTNCNN
CNTNCNTGTNTATGACGTTACCCNTGNGATCTGTAGTGNTTNNGCATCGNGTNATGTTNATGNGNNAN
NNTATNNCTNCNATNTTCNCACTNTNNTACTTTNGTGACGTTNNACGCGAANNNANATTANNNTATAN
NGNACTNANCCNNCCGATNTNTNNTACTNNTGANTATCTCTATCNNTGNTNTNTTNTACNATANATGN
ATNNNTGNANACATGNCTATNACNNGANTGNATTAAATNTCTNNCNNCG
TTTANCNCNNNNAGGNNNNCNNTTTTNNANNGTATCNCACTACANAACTAGGGGGATCCCCCCGGGG
CTGGAAAGGANTCCANGNGGTCATACCCANGGTTGGNAGATGAANGGTTATTCAGGNTGCCCGNNNC
TTCTGTCNACGAGCCNANTCCTTGGNCAACAAAATGANNTTTNAGGACNTGGTTNAGGCAANCCANCA
NNNTCAACAAATGGCCATCCCCGNAAAGNGAAAAGGGCNANAACTCTTTTCTGCTTAANTTAANCTTT
AGNNAANCCCACATNGCTCNTNNCCCCNANGGGGNAANCTGAACCATTTTTTTNNNCCNCTNNATTTN
NGGGGNACCTNATTCNAANATGTNTCTACCGGNNNAAAAATTTTTTTTTTAAANAAANCTNTTNTNTG
NNCCCNTNTTNCAAAAAAAANGCTTAGATTATAATCNTTCTNTGGCNNCTGNNNTNTNACGAAAAA
NANNCTCTNGNTTGTTAAGAANTNNNANAACNCNNTNTNAGTCNCNTNTNNGANAGNNNNTACNNAN
GGNGGTCNCTCNNCNNNTNNNNNTGGCGGNGGGANATATANACNANCCNNNACATCGCCNNNTNTCT
TNNGAAATNNGNNCNCTTNCCNTCTNTTNNTTTTNATTTNAANNANGTTTNCGTTNACANANCATTTTN
TNNNNTNNNANNNNACCCNCNNCACTCTNTNNNNTTTTNNTNTANAGNNTGAGACTTATNTTACANT
TCNNATATNTTNGNACATNTAAATCTTCTTACTTCCCANTGTGCCTNNTTACNCNCTGTGNTNNTCN
TTTCNCNANANGNNANCTNTNTTTNATNTTTTNTTAANACTCTNGNCNANTAATCNCNAANACANTNG
CCNCATNACAGCCTNCTCNANATCNCGNTNACATANANTGANACTTGTANTNCNCATGAANTGNANNT
CNNCANTANATTCATATNNTTAANTTGACNCCTCTTATCTACTANCGTCNNTTNANTATANTTNNTCGA
NTCNNNCTNTANNTNATCTACANCGTNANNACTTTCCTANTAANNCATTNNANCNCCTAANNTCACGN
CNANGANTAGTTNCCNCNACNNTCCC
GGTCNNANNNAGGGNNNTANNCAGNCCNTCANNNNNNNNNNNNNTTNNNGCCCCTCATNGTGGNAN
TACGGTATTAACAAACGTNATTACCANNTACANNNNNNNCGGCGNCTATNACCTGCNNTNCTCTCAC
TNTNCCACCCCCACTCCCNCCTACNTNNNTTNTNTNTCNTCNNTCNANNTNCCNNNNTNCANTNTTCCN
NTTNGGCGGNGNTCCTCCNTTCTCNCTCCTCNCCTTTNANCNCTACNCCCTCTNCGNTTTTCGCATCCN
NTNTCTTNTNCTNCTCNTNNTNTTCCGNNNCCCCCCNCCCGCTCNCCCNCTCCNTTNCTNCNGTTNCCT
NTTNCTCCTNNCTNTCCACCNNTCTTCCCCCTCGNCATTCTNCTCCTNCTTCCTCCTTTCTATTNTCNGN
CCCCCNNNCNCCTTTACTNNACTATTTCNNTNATTCCACATCNNNNCTNCNTCTCCNTCTTTCCTCNCA
CNNATTCCCCGNNNCTGCCCGCCNNCCNNTCCNCCTTCNCGTNCTCCTGTANNTNTTGNCCCNCNATT
NCCCCCTCTTTNNCCCNTCATCTCCCNNCCCCCTNNTNTTCNTCTTTNNCTCNTNCTNCCGCNCGCCGC
CCGTTCTNCNCCNCCANNCCTNCCNNNTNTCATCTNCCCCGTCNNNTNTGAANTCTCTNNCCNCCNTCN
NCNACCCNTCCCCTCGNTGCCCNCNCCNACCTNCGNTCTCNCCTTACCACTNNCTCCNCCNCNNNNT
CTANTCTCTTCNTTNTCNACNNCNTCNGCCNNCCNTNCCNTCTCTTNTNTCNTATCCTNCNNCTCNCCC
GCCNTCCATACTTCNCCNCNNCNGCANCNGCCACTTCCATCTNCTNCNNTCNTCCNNTTTCCGCGANTG
TACANCTTCACTCNACACTCCCCNCNTTCNNCNNCNNTCTCGCTCCACCNCACGCCNTGANTNTGNAC
NNCNCTACCTCCNCGCTNCACCCNTCNCNCCAGCNCNCTCNTCNCTCNTCNCTTCATCTCNCCNCCTAC
NCCCCNTNCNATNTNCTCCCNCTCGNNNGCNATCTNCNANCTTTCNTCANNCTCTCTNTCCCATGCTAA
ACCNCCTCNNCTCNCNTATCNNTCCGCTTCTCCNTCCNCTCCNCTNTCCGNTCGTGNCCCCNNNGGCN
TCNCCTCCCGNCNTNCTNANCCTTCGTCNCNNGCTCANCCNCTTACTNNCTNTNNTTCATATCNTCATC
TNACCGNNNCNGCATCTTCTNNCATNTACTTCTATTCTATNNCCNCCACCGNTATTCTCCNCCCACAGN
NCNCNTACTCTCACNNNCTTNTCTCACATCTCTCGATCNNCTNCNCCTCACCGCGCNCTGCCACGCNNT
CCTCNGNCCTCTTCTTACCCGCCNTCTACANTCGACTCTTCGCCCCATCTGTCTCNCTCTACCCTCTACT
CCTACNNTCNTNNCANTGTATCTNCTCACNTNTNNNNNCNTNANATATNNNCTANACTTCCTNTTCCTC
TCTCTANNCCTCTANAGTCCTCTTCTCNTNGCNTCCNACNTCCCTNNNCTCTNCNTGCTCCCATCGAAT
NNNCGNNNNTANCAACCCCCCGNNNTNATTCGCG
TTTGAGCCACCATNGGGAGNNGCNTTACAGTGNNGGCCGCTCTAGANCTAGNGGATCCCTCGGGCTGC
AGGAAATTNCAGCACGAGGNCAATTNTGNTTCACCANCAGCTCTACTGNAGCCNNGGCGATAAAATT
CGGCCAGGGTTCTCGNTGTGCCGNCTCTGCTCCAACCGGCACGGTCTGATCCGGAAATACGGCCTCA
ATATGTGCCGCCAGTGTTNTCCGCCAGTATGCAAAGGACATCNGNCTTCNTTAAGGTACGCTGGCGNA
CGCCATGGNCTCCTGTTNCAGGANAGGGNCNACAANCCGGNAGNTCGGCGTCCGNGNTTGATTCAGN
GCCGTTCATCTCTTTCAGCTAAGCNGGGCNTCTTNCTTTGTAAGNCGGACAACAACAGCNGANAGGCT
CGNCAGNCATCTTAATAGGANTTGAGGGGGANNGTGAAGGCTNTGNAAAANGTAGATNTTCNTAATA
CCTNTTTACCCATNTCTGNNAGNTGTAGGTAGCTATATGTAACNGTAGGATANACNTTNCTTTTNCCGA
ANTCCNTTNNACATGAAAACNTTAAATCNCCTAATCATATCCTTNGTNAANTTTNNCGNCAAAATAAC
NTGCCCTCTGAACCGGCTTGCCCTTGAACNTAACCTGAANNAATGGTCAATGTNCCTCGANCCNTCNA
NGGGTCTNGAGGGCCCCAGTAANACCCTTCTTAGGAANNCCNNAACGCCCNNNCCNTTCTAATNATNN
CTTTATNGACNTCTNNGTCNNANTCGAGTTCACAAGNNTAAANACNTTNTCCCNNGGGNANNAANNC
AACTANNNTGGNTTCNCTTNCAAAACTTANTANNGTCANNGNNTTTTTTGGNCNNNTNNAACGTTCTC
CTTCNNNGTNNTNNTNGNTTTTNNTNTNTANNNCGCNCNAANTAANNTTCCTCANCNNGTGTTTNNTT
CAGAANNAANANATATNGANGCCNCNATCAACTATGCTANTTNNNTNCTCANAANNCACCCTTCATN
GTNNGAGACNACTACCTNTATTTNTTTNTCG
TTTGAGCCACCATNGGGAGNNGCNTTACAGTGNNGGCCGCTCTAGANCTAGNGGATCCCTCGGGCTGC
AGGAAATTNCAGCACGAGGNCAATTNTGNTTCACCANCAGCTCTACTGNAGCCNNGGCGATAAAATT
CGGCCAGGGTTCTCGNTCTGCCGNCTCTGCTCCAACCGGCACGGTCTGATCCGGAAATACGGCCTCA
ATATGTGCCGCCAGTGTTNTCCGCCAGTATGCAAAGGACATCNGNCTTCNTTAAGGTACGCTGGCGNA
CGCCATGGNCTCCTGTNNCAGGANAGGGNCNACAANCCGGNAGNTCGGCGTCCGNGNTTGATTCAGN
GCCGTTCATCTCTTTCAGCTAAGCNGGGCNTCTTNCTTTGTAAGNCGGACAACAACAGNGANAGGCT
CGNCAGNCATCTTAATAGGANTTGAGGGGGANNGTGAAGGCTNTGNAAAANGTAGATNTTGNTAATA
CCTNTTTACCCATNTCTGNNAGNTGTAGGTAGCTATATGTAACNGTAGGATANACNTTNCTTTTNCCGA
ANTCCNTTNNACATGAAAACNTTAAATCNCCTAATCATATCCTTNGTNAANTTTNNCGNCAAAATAAC
NTGCCCTCTGAACCGGCTTGCCCTTGAACNTAACCTGAANNAATGGTCAATGTNCCTCGANCCNTCNA
NGGGTCTNGAGGGCCCCAGTAANACCCTTCTTAGGAANNCCNNAACGCCCNNNCCNTTCTAATNATNN
CTTTATNGACNTCTNNGTCNNANTCGAGTTCACAAGNNTAAANACNTTNTCCCNNGGGNANNAANNC
AACTANNNTGGNTTCNCTTNCAAAACTTANTANNGTCANNGNNTTTTTTGGNCNNNTNNAACGTTCTC
CTTCNNNGTNNTNNTNGNTTTTNNTNTNTANNNCGCNCNAANTAANNTTCCTCANCNNGTGTTTNNTT
CAGAANNAANANATATNGANGCCNCNATCAACTATGCTANTTNNNTNCTCANAANNCACCCTTCATN
GTNNGAGACNACTACCTNTATTNTTTNTCG
TTGANAANANCTGGAGCTCCACCGCGGTGGCGGCCGCTCTAGAACTAGTGGATCCCCCGGGCTGCAGG
AATTCGGCACGAGGGCTNTACCANCGAGGCAGGAACGTGGACGTCCGNGGGGTACTCGTTTGCACCG
ACGAAGACACCTCTACTGCAANGATGGTNAACGTNCCTAAAACCCGCAGGANTTTCTGTAAGAAGTGT
GNAAAGCATCNACCTCACAAAGTGACCCACTATAANAAGGGCAAATATTNCCTGNNTGCCCANGGAA
AGAGNGCNCTATNATCNNAANCNAAGTGGCTACTCTGGNCNANCNNAGCCAATATTCTNNANTAAGN
NTNATANACCNCCACCAATNANCGNGCTTAGACTTGANTGCTTTTGANCCCATNCTNNCNTNTCCCNN
CNGGATNCTTNTCCCATCACTAGNNTCCCNGCATNTTTGNAACTTNTANNNANTANTAAGANANANNN
NNCNGGCCNNATNTGATCACAGNTTNTCNNTGCTTGNCGGAANTNANTTNTNANNNCTTANTNNTTCN
AANAAAGAAAATTNCTTGAAATTTNANNTCATACATCTGCNCTNNAATNANNANNTACGANANNNNC
CCATANTTTTTTTGTNTCGCANNNNNNNNTNNATATNTTCTNNCCCTNNTNGNGNTTTCCTTCNNTTCC
CNNGTTCTTNNNNTTNNTATNNTTATTTNNTNANCCNCTAANTANTCNNTNTTGNNTNNTTTNCNTTGC
NCCCNTNNNTTNTCTNNTTNNANTCCTNNTTTNTTTTTNNCNCATGTTCCACTANTTCCTCTTTNTGTTN
NTATATTGNTCCTANCNATCCTNTNTTTNNTCCCTCTTNTTANCNAGNGTGNTTACNTNTNTTNNCCNT
TTANNNCNTGNTCACCTTNTNATGNNNTCNTCNTNNCTCNTNTTCCTACNNCTCNTNTNNNTCNTCTCC
NANCNTAACNNNATCNTTATNTTTGNNATCTNNTACACNTNTGAACANCCNTNTCACCATTGTNNATC
NCTCCTTNTCATCTNCTNTNCATNTNNNNACCATCNNNTTNATCCTTCNNTCNCACCNCNCT
GNGGTTNAANAAANCTGGAGCTCCCCGCGGTGGCGGCCGCTCTAGAACTAGTGGATCCCCCGGGCTGC
AGGAATTCGATATCAAGCTTATCGATAGCGTCGACCTCGAGGGGGGGCCCGGTACCCAATTCGCCCTA
TAGTGAGTCGTATTACAANCCTGAANAGNCTTNAACCTNCTANNTGNCGCTGAANAAAGNCANGACG
TCAANNNNGATCANGAGACAACCAATTATACTACCGTACAATGTAANANNCNCANTCNATTCCCTATA
NTGAGTCTTCATAACAAATACCAATCATAATTGGAGGATTCGGTAACTGACTTGTTCCCCTAATAATTG
GTGCTCCCGATATAGCATTTCCCCGAATAAATAATATAAGCTTCTGACTCCTCCCTCCCTCATTCCTACT
ACTCCTCGCATCCTCTATAGTTGAAGCTGGGGCAGGAACAGGCTGAACCGTGTACCCTCCCTTAGCAG
GCAACCTAGCCCATGCAGGAGCTTCAGTAGATCTAACCATTTTCTCTTTACACTTAGCAGGAGTTTCCT
CAATTTTAGGAGCCATCAACTTCATTACAACAATTATCAACATAAAGCCCCCCGCAATGTCACAATAC
CAAACCCCTCTGTTCGTATGATCCGTAATAATTACCGGCCGTACTACTACTACTCTCGCTTCCTGTATTA
NCAAGCCGGCATCACAATGCTATTAAGAGACCGGAACCTAAATACAACCTTNTTNGACCCCGGCAGGA
NGANGANACCCTATTCTATATCAACACTTAATTTTGGATTCTTTGGGACCCCCCGAAATCTTATATTT
TNAATCTTAACCTGGGGTTTGGGAAANAATCTTCTTATTATCGGGGAACCTACTTACTTCANGGAAAA
AAAAAGAACCCNTTCNGGATATATTGGGGAANAGGTTTGGGGCTTTTAAANGGGCAAATCGGGAANT
TTTAAGGGTTTCAATNN
TTGAAAANCATCTGGAGCTCCACCGCGGTGGCGGCCGCTCTAGAACTAGTGGATCCCCCGGGCTGCAG
GANTNCANCCNTNGGNTTTNTNNCCGATNTGGCTGNCATNGNGANGCCCGGGGNAGNTTCNAATATG
AAGANNNCGGNTACNCAANATATNAATCCACTGCCTTCNAATGAAACGATNTGAACANGAGAAGCAA
AGCANTGCNATTCNTAATGAANCCGTGCGCNCGNCCGTCNGCACTGTNCCATNTCCANNAAAGCATNT
GCCNTTNTTATTTTTAACTNCTTTTNACNCTGCTAACCTTNGTTAANTANNCTNAAGGAGGGTTGGAT
NTGAGCTTNAAATTGACNTGTGCTAGNCCCTTTCCAGATCANAGANNGTGGAGAACTACTGNCAACG
TAGGCCGCGCCTGCCTCTCCCATCTGNNTAGTGTGGCTGNCACGGGAAAGGAAANNAANTTGCATTGT
NTGGTGANAGGACNGANTNCTGTTTGNTTACCACNGTGAAANCTACAGNTAANAAAGCTGGNCCAAN
GGNCGNTNCTNCTNTNCCTGGAAAAAATGCACTTTTNAATCAAAAGGTGCCTTTTNTTTTNTTNTTCNT
TNCNAANNATTTTTAATTNATTGGNAAANGCCNCAAATNTTTTTTTNTTANTGCANAAANAAAANTNC
TNAAAANCCNTGNNNCCCANNGNNAAAANAAAAACATNNNGNGGGGGGNNNNGGGTTCCCCCATTC
CCACCTTNTNTNNGNCTCNTTNNNANAAATATAACCAGCCCCCGTANCCTNNCTNNTNTNCNNNNNCC
NTNTCNNCCANTTTCCTTNNNNCANTNNTCCNTTNTNTNCCNNTCTNTTTTTNNTTTCCCNNTTTTNCCN
NCNTTNNTTTANNCTTGCANNCNTCGCCNTNANACTTACNNNCTAATNCTTCCNNNCCCAAANCTCCC
ANCTNNCNTNTTCTTANGCANTNANNTCCTNATCCAANCAAGACNTATTACACCNTTCTNTTACTNNTT
TNTTACTNTCTCANCCTNCNTTATCTTACACACTCGNNTCTCNATTNNCANAGTCNANCNTNACCN
NACAAAACTGGAGCTCCCCGCGGTGGCGGCCGCTCTAGAACTAGTGGATCCCCCGGGCTGCAGGTT
CGGCACGAGGGCTGACAATGACATCGGAGCTGTCTCAACGACAGGGCACGGTGAGAGCATCCTGAAG
GTGAATCTGGCCAGACTCGCGCTCTTCCACGTTGAACAGGGAAAATCACTAGAAGAAGCTGCCAACGC
ATCACTGGGTCATATGAAGTCAAGGTCAAGGGCGTAGGTGGTATCATCATGGTCAACAAAGCAGGA
GAGTGGGCGGTGAAGTGGACCTCCACATCCATGCCCTGGGCAGCGGCAAAGGATGGCAAGCTGCACT
CGGGAATTGACTTCGGCGACACGAGCATCATTGACCTGTCCTAAACCCCCAGAAGCTGTATTCTAGAA
ACTAGCTTGGAGGTGACGCACGGTCTCCTGGCGTGNAGACTTANCTTCATCAATTAGATCTAGAACTA
GAAAAATTCTGCAGCCTGTCACTCGTTTTGTTGCCTNGATCAGTGTCTGGTTGGGGGCGGGTTCCAAG
TGTGTGAGANACGCCCATGTTAAGATAAAAATAAGACCAGTGAAGATGACCAACTTCACGGAGCCTN
CCTTATGAAATAAGTCCCCATTGTCTTGAGATTAGAAAGAAGAAACCNGCAAGATCTTAACAGTAAAG
AAAGGGCCTCGATGCAGGGAACGGGTTTGGAAGTTGGGGGACCTACAGCANTAACAGAAGCTGTANG
TTCAAGGGTGTGGGGGGTTCGGANGAAAAGGCTNTGGGGCCAGGACCCATGCAGGGCCAACAGGCNG
GAAGGANCCNNNGGGACCCCGGGNAAGGGACCCGGGNNCCAAANTTATGGNACCCTGGATTGTTGCA
AACTTANTTTAAAAGNCGGAA
NGCNNCGGCGNCGCTGCGNTCNTNNNNNNNNNNNNNNNCCCNNNNNTCCTCCNCCCCCNCACTTTTN
CCCTCNNTCCTCCNTCACCCNCCCTNCCCNANGGGNGGNNAGTNNGTNNGGNATCTTTCCCTTTNAGN
CCCCATAGGAGCCGCATNACANGGCCCCCGCTCCNGCATCTAGTTGGNTNCGCCCGNCCTGCCANGCA
NTCTNGATAANTATGCATTTTTTTCTCTTCTCCTCCGGNGGNCNCNCNNNTATNTCANGNCCTCNTTAT
NGATNAACCNNNNNTNNNNTNTNCNTNATNNGNNGNAAACCNTTANTCNNGATTTNNTATGNNNNNA
TNACNTNTNCANGANNNCTNGANNNTATNCTNGCNTNGNANANAATCCANNTTAAAGGCCNAGNNNN
ANGAATTTNAATNNNCNGCCAATCANNTNNNTNANNATTANNNNNTAAAANCCCNAAANCTGGCATC
NTCNNNNACTACNTATTGCCTTNTATNANNGNCANGNTAAANNNNNNATCTATNAANGNCCGCANGC
NNNCCCANATNCNTTATACGNGTNCNNAAAATNNNNCGGGNCGATNCNACNTCNCTCNCNCANANTN
TACCTCNGCTNNNANTTNTNTATANNNANTTNGNANNTNTATGNNNTNNTNANNNTNATTGANNNTN
NNGNNGGANAGGNGNCNNNATANNNNTNCNTTTNCGACNNGCNGCNCNNNGAANTCAGCCATTCTNA
ATTCNNCGNNCGTTTANTAGNNTCGANTTCNANTNNAACTACGGTCCCTAAACTCCCCNTNNNTANNN
NGTGANTNNNTATCGTTATTNATCGCGACNCCGANNATTTNTTTGTACTTCGNCANNCTANTCNANGG
ACNACANTAAATTTANCNCNACNNTCNGATANNTTTANNTNGNTGNNACNCANCNCTATNNNNTATN
CGTTACTTTACCTCTNCNGTCGGAGCGNCANGCCTANNTCNTTCCTANTCNTAANCCTNTTCGCNNNNC
NTGACNCNAACNNCNTGANATNNTTTATNCATAANTTANGTNNGCTATACNAACGNNNANTTNCTNN
NCACGCNGAAANNNCNTNCNCTCTNTCCTNTNACTNANCNNNTCCNGAACCANAGTGTNNGTNANAN
TGTNCATNTTNTCNGTNANNCTNTCNNNTCAACTTNTNCATATNCNNGCTGTGGATGCTCCNATNTGN
NAACAATACGNACNNCCNCNTGGANTCNNCNANTGNNCGNACTNTANACNGCNACNCTATAATNNAC
GNCGNTNNCTTCATACNCNACNGACNTATNATANCGTATCANNCTNCANGCCAGANNCNCGNCATAG
NGTNTNNGATCACACCTGCNNTTNGNCATANCGCATNTACNTGTNCATCCNACCNCCATATNGTNTTT
ATTATTCGCNNTGTGGATNCACACGAANANANCTCGNNAGTCTCATNANNCTNAGTNTCATTNTCTNT
ATCTATNATGNCNGNACANNTACGTCGATATACGNNTGCTGNNTTATGTTGTNTTCGACATNATGTNG
NAATGCTAGTGGNCACTNNTTNANNTGTGNTCTTTNATACTACGGAGCANNANACACACACTCNATAN
AGTANTGATATCACTAGNTCCC
TTTTGAAGGNCCCNTANTNNGTGGANGTTCGGTCATTTAACAAGGTGTTNTTTCCCANANNANTACAC
NCNAGCNGNTANNNNTNNTANGAGCATTCTNATTNCTCACTNTANGTNNTTAATCATNACGTAGNTNT
CTCCGAGANCCTGCNANAATCATNAGCNNNACNNTANTNNTANACGGGGCNNTNACNTGNCTANTAG
TGNNCANNNCAGNANAGNATNANAGGCGACNCNTNGANAGGACGTTTNATGNGACAANAGGANTTG
AGAAGCTNCGAATGNGNATCGCAANNGNGGANNNATGACACTGNNANGACGACNATATGAGANNAN
ANTNNNNNNNAGTNCNNGNNTNTANNTANGTAGTCTTNGNTGTTNTNGNTGANCNNANGCTNTNGGA
GAGTNNANNNGNAGNTGNCGTCTTGTNNNNTGNGGANNGGANTGNNGAGTAGNNATATNNACTTCGT
CGGTTTCNNGTGNCTACGAGACNANNNCGANTNCATNNGNNATNTNGTGAGNNTNANCTNGTCGAAG
TTNCNTCNNAACGNNTATNNATNCTCNTAAGACANTACANGATNNNGATNGTCGATGANATNNNTATT
ANTNNCGCAGCNAGGNGACTCGNGTNGTCGTNNTGCANTCNNNGNNTGNCNNNNATNANGAGTANTG
ANNANNNNNTNCGAGTNNACGACACTAACNNNTTGAATANNCTNNANTANTNNNAGCGATGNTNNAA
CNTTTAATNGTCNGCANATNTNTNNTNAGCANNACNGNNACNAANNGATGACGCATGNNNTGTGNNC
NNNTAAGTNNTNNTGTNNGNNNNNCGTCACNNTGGANTNTTNNNNTTNNNTGNNTCTNCNCNCNNTCT
NGTTNNGTNGGTANNNGNGNAGAAGANCTCAACNANCNCTTACNATNNTCTTACNTATNGTCNGNCN
GATNNNTGNGNNTNNAANCGNGANNATACTGTNNNAANTNCGCANCTGNCNATGTANCNNTANNNNA
CGANTTNNANATNNTNCAANNNTACGNANTCNANTNNTTNNGNNTACNTNTCGTNCTNCTNANNGNC
NATNANTGANNACGGAANNTCNGTNTATNGGGGNNTGGTNGACTTNAACNACCNNNNNGNGAATTNG
ACTACNNTGGANGAGNTNNGGAAGGNACTANCNCAATTGCATANNTNTATNAGAGNCNTTGNTTNNN
TNTNCGTAANTGTATNACGANACCTAAGGGGTCGNTGACGNGTACCNGTGNGTNNNCGACAGNANGT
GNCNGACGNNNTANGGACGCNTCGANATAGTANNTNTNNATTGCNANNNTAGACNNCNNNTAGGTNA
TANGNACNGNNGGANNACNGTANGATNNNCNAACGNANCAACNTTNTGTAGTNTNCTTNNCTNNANG
GANCGANNAAANNNNGACNCTNNNAACNGAAGCAANATCNAAANTGAGCGACNTATGTACGNNACG
NATGCACNNACGGAATGCNAACGNTCNAACGGAAAGNTTNNNNNGNNACGTATTTAANGTNNGTNNC
GGNAGNGAANNGTANGTAGNGNACGNNNTCGAACGGATGCNAGTAATACGNANNTNTGNGACGTNA
ATTAGGCNACGN
GNCCTANTAGTGGAGTNGTATTNCATCGTATTACACTCTANAACTAGTGGATCCCGCGGGCTGCAGGA
ATTCGGTCACGAGGACCANTGAGAAAGATAGGANTACANNGTGTATTAGGNNANCCNTCATTCTTGCA
CTAAGCACCAGAAGACTNGANAAATCTAAAAAACAAAAAAAANANCTCATTNAACTAGGATAATTCA
AANGGAATATTAAATCGATAAAACCAGGACTCACNTGCCAAAGTNCNCATCANGGGACGCTCACATN
TNGTTTCCTTCCATATAAAACTTGTNNGCCGCTATATTNAAAGCCTTTTTTCCAAATATTTTCATTGANT
TTCTGCNAGATCTANAGCTCTGCTCAGTGCCTCTACAAAACAGNGTACATTNTTATACTGCNTNGAN
AATCCGCCCGGTAANAANCATGTGCANCTNTGCAANANTTTTTGCTCAAAAACATNANTGACCTGAGA
GCAACTNNCCTTTCTATTCCTAATTGCTACCTACAGGACCAGCAGGGAGAANTNTCCCCTNTTCCCCNN
CANAGAATACTGTNATGCAATTTACNNNNNGGATGCNTNAGGCCTTGNTGAAAATTGANGCCTCACCC
TGAAATCNATAAGCCCTCCCCAGGGGAAGNNAATTCTCTNCNGCTTCTGTNCTAAANTNGTNCCNCNT
NCCCTAGTNNGTACANTCCACTTTTNCGGGCAATTTTTNATTAAANCAAAAATNCTTGGTGTNCTTTTT
CNTAGAACAANCTATTATATTCATTTTTCCCCNCATTTNCTANTNAAAANCCCANNNGGTNTAGGTTNN
NTAACCCTCTAAAAAATCTGNCACTTTTCCNGGGCNAANAAAGGNTNACTACTTTTTCTAAGGNGATA
ATNCAANATTCCNTTTNTCTACTNGTGTGGGNNTTAANCNTCTCTTTTAGGCAANGNGNGTCGTNATA
AAAAACNGNCTNCTCTCCCCAANNGTNNNTTGNCNCNTGNNTNANNNTTTTTCGATATTNAANAANTN
TTTCCCCCCNGACCCCNGCG
TTGAGNCCTNNTAGTGNNGTNGTATTACANGTGNNGGACNNTNTAGAACTAGTGGATCCCCCGGGCTG
CAGGAAATTNCGGCACGAGGGCNANNTGGGTCACCAGCAGCTCTACTGGANCCNTGCTNTAAAATTC
GGCCAGGGTTCTTNCTCTTGCCGGNTCTGCTCCAACCGNCACGGTCTGATCCTNAAATACNGNCTCAAT
ATGTGCCNCCAGTGNTTTTCCANCTGCATAAGGACATCGGCTTNNTTAATGCACAGCTGNCGGACNCC
ANGGTCTCCTGTTTCAGGGAGGNGCGACAAGCCGGTANGNTNGNCGTCGGCNNTTGATCATTTGCNCG
GTCTTCTCTNTCCCTAGCAGGGCTNTNCCTTTGTNTCGGGACAACACANTTNANAGGCACCGNGNTTC
NTTTNAANANGANTGAGGAGNGANGNGANNGCCNNNCANCANNTAGTATNNNNTAANACCNTTTTAC
CCATTNTNTGCNAGTTTNTAGGTGCTATATNTCTATGNGANNAANAAAACNTTACTTTTTCACAANTCN
CTAATAATGANNCCNTTNANTNCCCTTAANCATTATNTTGGGATAATTTTTTCNCCNTTAANANCATGC
CTTTGAANCGTTTTTCCCTNNTTATTCACCCGANCTANGGATCATGTNCCNCNCCCTTGAGNGGTNTNN
NCTCNTGNANTNNACCCNTTTTTAAGACNTCANAAGGCTTCNCNTCAATNNGACTATGGCCTTCNNNA
ACTTTTCGGCCCTAAGGTCCNTTAAANGNTNANNCTTTTNCCCNCGATNNAANAANTGANNTNCCTANG
GNNTCNATTGAANANAANNNCCTTTTGTNAAACCNNTNTTCCTTTACNTCCTCAAATAANCAACTTTN
AAANTTNNANNNGCNNTTNTTTTTNNCTTNNAAAAAANTAANNAACNNCCTNGGCCCCNTCTGTAAGG
AAACTAAAAANCCCTGCCCCNCCTCTCTTNTNTTTATTNNCCAAGTNNTNNACGCNTCTCNNTTGNGTN
NAATATTAAAACNCTTGTTNTNTNCG
TTGAGNCCTNNTAGTGNNGTNGTATTACANGTGNNGGACNNTNTAGAACTAGTGGATCCCCCGGGCTG
CAGGAAATTNCGGCACGAGGGCNANNTGGGTCACCAGCAGCTCTACTGGANCCNTGCTNTAAAATTC
GGCCAGGGTTCTTNCTCTTGCCGGNTCTGCTCCAACCGNCACGGTCTGATCCTNAAATACNGNCTCAAT
ATGTGCCNCCAGTGNTTTTCCANCTGCATAAGGAGATCGGCTTNNTTAATGCACAGCTGNCGGACNCC
ANGGTCTCCTGTTTCAGGGAGGNGCGACAAGCCGGTANGNTNGNCGTCGGCNNTTGATCATTTGCNCG
GTCTTCTCTNTCCCTAGCAGGGCTNTNCCTTTGTNTCGGGACAACACANTTNANAGGCACGGNGNTTC
NTTTNAANANGANTGAGGAGNGANGNGANNGCCNNNCANCANNTAGTATNNNNTAANACCNTTTTAC
CCATTNTNTGCNAGTTTNTAGGTGCTATATNTCTATGNGANNAANAAAACNTTACTTTTTCACAANTCN
CTAATAATGANNCCNTTNANTNCCCTTAANCATTATNTTGGGATANTTTTTTCNCCNTTAANANCATGC
CTTTGAANCGTTTTTCCCTNNTTATTCACCCGANCTANGGATCATGTNCCNCNCCCTTGAGNGGTNTNN
NCTCNTGNANTNNACCCNTTTTTAAGACNTCANAAGGCTTCNCNTCAATNNGACTATGGCCTTCNNNA
ACTTTCGGCCCTAAGGTCCNTTAAANGNTNANNCTTTTNCCCNCGATNNAANAANTGANNTNCCTANG
GNNTCNATTGAANANAANNNCCTTTTGTNAAACCNNTNTTCCTTTACNTCCTCAAATAANCAACTTTN
AAANTTNNANNNGCNNTTNTTTTTNNCTTNNAAAAAANTAANNAACNNCCTNGGCCCCNTCTGTAAGG
AAACTAAAAANCCCTGCCCCNCCTCTCTTNTNTTTATTNNCCAAGTNNTNNACGCNTCTCNNTTGNGTN
NAATATTAAAACNCTTGTTNTNTNCG
NAAAAGCTGGAGCTCCACCGCGGTGGCGGCCGCTCTAGAACTAGTGGATCCCCCGGGCTGCAGGTTCC
TGTTAACTCTTAATAAACTTACACATGCAAGCATCTACACCCCAGTGAGAATGCCCTCTAGGTTATTAA
AACTAAGAGGAGCTGGCATCAAGCACACACCCTGTAGCTCACGACGCCTTGCTTAACCACACCCCCAC
GGGAAACAGCAGTGACAAAAATTAAGCCATAAACGAAGGTTGGACTAAGTTATATTTAATTNGGGTGG
GTAAATCTTCGTGCCCGNCCCCCGGGGGCCNTCCAATAANCCCANNTNACANGNNNTCCGGGTTAAAC
GNGGTAAAGNNCCNTACCNAATANGGGTNAATTCTTACTNANCTGGNAAAAGNCNTGNNTTAAATNA
AAATNAATGNCGAAAGGGNCCCTNCCATNNNCGNCNCNCTNTNNNTNANANCCNAACTGGGANTANA
NNCCCCANTTTGNTTANNCCTTAACCCCGNTTATTNCCTTAACCAAATTATTCNGCNGANNNCTNCTNN
CCACNGNTTNAAACTNAAAGGACNTGGNGGNGNNTTATTTTCNTNTAGANGNANCNGGTNTNATATN
GATAAANCCCGNTAAACCTTANCCAATTNTTGGTTATACCGTNTATTTACCGGCATTTTNAACNAANCC
TTAAAAAAAAAAAAAAAAACTTNGGGGGGGGGCCCGGGNCCCAATTTCNCCCTTTAGNGGNGGNNNT
TTNANNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNC
ANCAAAGCTGGAGCTCCCCGCGGTGGCGGCCGCTCTAGAACTAGTGGATCCCCCGGGCTGCAGGAATT
CGNNANCAAGCTNNNCCCTACCNNCNACCTCGNAGNGGGNCCCGGCCCCCTAGGANTCCCTATGNCG
AGTGGNTATTACAATCAGGTACAACTGCAAGANANNACNTTGGNNNNNANCCAAGCTGGNTCTATAN
CAAGCAANCTGNNTCTATGATANNGGNTCTTAACATGATGACCCTNTTNTAGAGCANCCAGATCACAA
TGAAAACATCNCTNCTGTANNGNTCTGGCCCATGATGNTATNTCCTCANTATCNNTCTGACTCCCAGC
AATNAATATACAGCGTNGCANAAAAAACAAATNCCACTTGTGGGGGGGCCTGGNGACCGAAGTAANC
CTTAGATGNTGTNAAGAAACAGCCTTGCATACGNGCCTGGAATGTTANCTNCCTGAATCAAGGTNANT
TGAAAATGNTCAAATNCGACATGCTCGAGNGAACATACNCATTTTACGAACTCNGGGGACCTNAAAT
GNACTGGGAATGGGTGAACTTAACTTCANATAACCATTATTTCTACTTCNGGGGGNNGGGAATTCTTT
ATAANAAANGGANTTNCNTTATGGCCACCAAAAAATTTTGAAATGCANCTNTTGGGTGCATNCTTAAA
AACGGCGGGAAAGATCNCTGGTCATTTCCANGGCCNTTCCNTTCANTATNACANAANTTCCAAGGCTT
TNCCCCCCCCCAANTNTTCTTTGTTTAANCTCCGNNTTGAACAATTTTTGGGAAAAACCTAAANAAACC
CTTGGGGAACTTAACAATCGCCNNAAGGAATNTCCTTTTTTCCCNNACCNAGGGANNNGNANCCCCCG
TTTTTCCCCAAATTTNAAGGGCGGGGNTTNTTTTTTTTGGTTTTAACCCCCCNGGGGGANTTCNCCCTTT
GGGGAAAAAAAAAAAAACCGN
TTTTGAANCACCATTCTGGAAGCTCCACTCGCGAGTGGCGGCTCGNTCTAGCAACTAGNGGANCCACT
CGGNCTGAANNAAATTCNAANCCCNANGATTTTNTNTCCCNCCCANTTACANCNNCAGCANCCNNNCT
NCAATAGGGGGGANCNTNTTNNNAAACATCNNNTTAAANCNCCNCCCCNNTCACATCNTCNNNCNAT
CTNNTTCCNNNCCTTCCANANNNANNTCCTCNNCANNACTAAANATCCNCCTANNANCANNTATNTAC
NTNNNNATAATNCCNNNTTTCCNTTTNTCTCACAATAATNCGAANATTATAATCATNANNCNCCNAAN
CNNCNACANANCNATNTNNNNCNAATTCGAAANNTTTCNNTNCNAANAGCNTAAACNATNANANNCAT
NNNNNNCNCNTNCNANANNTAACNACNNATNCAANTCNCCCCACCNNCNANANAATNCACCCACNCA
CCCTAAANNTACNATNNATNCNNCACNNTAANANNATTCCTTCTCACTANCAATTNATNATATCTATN
GNNNNNCGCTNCTANNTNNTNAAAANCTNNTNNANATNNCCAATCNTCACTCATNGCNTNCCCANTTC
GNCNTNAATNNTNCATTACNACCANNCGNNCCTNNCCNTCATATCTNNNTNCNCNATNCCNCNNNATN
CATACANANTCNNCANTGAACCNTTAATTNTNNNNTNACCNCTNNANNCNCAATCACCTANCANANTT
NNCCNCCTCNCNCTTNACTANACNGCCNNTCAANNCNCATCTNATCNANNANCCCCTAACTANNTNCT
TCCNNNTNCANCTCNACCCNAGNACCTATTAACAACNNNNAAANTNTNAATTACNTNTANNACNAAN
ATCTACAACATATCAGATCNNCTTTCNNTNACCACCTACCATANCTANTCNATCATCTTCCANACAATC
CTNNCCTCACCTNNNTCCNACCNNTCACCNNNCNCCACCAATCANCNATCACNNNNACCTNCACNANT
ATCTTCACTANNCAANCANNTCAANTTCTCTCTATTAACANCAATCCNNCTNCTTCCNCANCAAANAT
CANANNCACACNCANCNCNTTTNCANCTCNTNNNAACNGCCTAGCCCNCAATNNNNTCANCNTCCAC
AACANTNACTCACACATACNCNATACTNAAACNNNTNAGACNCACTTNTTNCNCANACCATTNCAGTC
CTACNCNNTNTNATATATCTNTGTNACNTANACNCAACNTCTACNANATACANTANACCGNTANTCAC
ACNACNGNNACACNNACACACACTCGTNNTANTACAANCGNCACCTCNTCTNNGNGNNNCANTCGTC
NCTACANTCGCC
TTNNACAAAGCTGGAGCTCCACCGCGGTGGCGGCCGCTCTAGAACTAGTGGATCCCCCGGGCTGCAGG
TTCCTGTTAACTCTTAATAAACTTACACATGCAAGCATCTACACCCCAGTGAGAATGCCCTCTAGGTTA
TTAAAACTAAGAGGAGCTGGCATCAAGCACACACCCTGTAGCTCACGACGCCTTGCTTAACCACACCC
CCACGGGAAACAGCAGTGACAAAAATTAAGCCATAAACGAAAGTTTGACTAAGTTATATTAATTAGG
GTTGGTAAATCTCGTGCCAGCCACGGCGGTCATACGATTAACCCAAGCTAACAGGAGTACGGCGTAAA
ACGTGTTAAAGCACCATACCAAATAGGGTTAAATTCTAACTAAGCTGTAAAAAGCCATGATTAAANTA
AAAATAAATGACGANAGTGACCCTACAATAGCCGACGCACTATAGCTAAGACCCAAACTGGGATTAG
ATACCCCACTATGCTTAGCCCTAAACACAGATAATTACATAAACAAAATTATTCGCCAGAGTACTACT
AGCAACAGCTTAAAACTCAAAGGACTTGGCGGTGCTTTATATCCTTCTAGAGGAGCCTGTTCTATAATC
GATAAACCCCGATAAACCTCACCAATTCTTGCTAATACAGTCTATATACCGGCATCTTCAGCAAACCCT
TAAAAAAAAAAAAAAAAAAACTTGGGGGGGGGGCCCGGGANCCCAATTCGCCCTTTAAGGGGGGNN
NNNTAAAANNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNN
GCCTATNNGGAGNNGNATNACAAGGGCGGCCGCTCTAGAACTAGTGGATCCCCCGGGCTGCAGGAGA
ACCTGAGCAAAGAGGAGCTGATAGCCGAGCTGCAGGACTGTGAAGGCCTTATTGTCCGCTCGGCCACC
AAGGTGACCTCTGACATCATCAACGCGGCAGAGAAGCTGCAGGTGGTGGGCAGGGCTGGCACAGGCG
TGGACAACGTGGATCTGGAGGCCGCAACCAGAAAGGGCATCCTGGTCATGAATACCCCCAATGGGAA
CAGGCTCAGTGCTGCAGAGCTCACCTGTGGAATGATCATGTGTCTCGGCAGGCAGATTCCTGAGGCGG
CGGCATCTATGAAGGATGGCAAGTGGGAGCGGAAGAAGTTCATGGGAACCGAGCTGAATGGAAAGGT
CCTAGGAATTCTTGGCTTGGGCAGAATCGGGAGAGAAGTGGCCACCCGGATGCAGTCCTTTGGGATGA
AGACCATAGGCTACGACCCCATCATCTCGCCAGAGGTCTCGGCCTCCTTCGGTGTGCAGCAGCTTGCC
CCTGGAGCAGATCTGGCCTCTCTGTGACTTTATCACTGGTGCACACACCTTCTCCTGCCCTTCACCACA
GGCCTGCTGAACGACAGCACCTTTGCCCAGTGCCAAGAAGGGGGTGTGCGTGGTGAACTGTCTCGAGG
AGGGATCGTGGACCGAAAGGTGCCCTGCTCCGGGCCCCTGCANTCCGGGTCAGTGTNCTTGGCGCTGC
NCTTGGACGTTTTTCACCGGAGGAACCACCCCGGGACCGGGCCTTNGTGAACCATTGAGAATGTNATC
AACTTGCCCCCACCTGGGCGNCCAGCCACCAAAGGAAGNTTAAAATCCCTTGGGGGGGAAGGAAAAC
GCCCTTCCATTTTTTGGACATGGGGAAAGGGGAAGGCCCTTGGCCGGGGGGTTTTAANNGCCAAGCCC
TTNCNGAATTTTTTTTCACANCCCCAACCTTGGGNCAATCTGGGTTAAANCTTTTNG
GGCCTATAGNGAGTCGNATTACNGGGGCGGCCGCTCTATAACTAGTGGGATCCCCTGGGCTGCAGGAN
NNAAAGATGCNCATTTACCAGACGGCCCNTGCACTAANAGACCNTGNTGGGNGGNCTTGNTCTCCGCT
GGGAANCACTNTNTGTAGAAANNCNNGNCNGAAACTANTATGTAGCCTGGANCACCTNCCTTTTNCCA
NGAGGGGATTTCATTNNTGTGGATTACTCTGGTTGTCCTNAAAANCANATGCCNNNNCATCNANGAAC
ANANNAGNGAGANNTNNNGACTNAGNTNTTTNACCCAGANCNACCNCGCTNTNGGATTCCNCAGATT
TGNATGCNNGNGCAGCCNTCANTNGCAAAATCNTNANGAGAGCTTNTGGCCGGNGAAANANNATNTA
ANATNANNGAATTTATCCCNGNTCNCGANTCNAAGNTTTGANNNNTNTTGTGNNTGATAGTNGNGTGT
TNCTNTNNANNNNCTNCNTTTTACTCNTTTCCTNNNCGATNNNNCGGGCNNNNNNNANNNCGTCNNNG
CCTNGGCTNTATAGCGTNTANTNNCNCTNTTNTCTNNNTNACANNNTNCGATNNANNNATNANNNGCT
CCGCTCTCANTGCNGGATTNATAANNNNTNTGNNCNNCNTNTTATANATCTTCNCNCTCANTNTTNNN
CCTTTTTNNCCNCNTACTNNNCTCTNCCGTNTCTTGNAANNANTNTNCNNCCCNTTTTTCCNCNTNATN
NNNTATTTCNNCNANNCNCTCCCCTGGTNGNNNNCNTCANTCTNCTGACTCNCTANTCTTNATTANTA
NNNANTAGTTACATNCNNNNNGTNATTNTCTANANTTNTANCTTGTCACGNTNTNNNCNCNCGTATNN
NTAGCAATCTCTNCAATNNNCAGTGANNNTANGTTCCTCNACGATCNTNCTCTCGACNTGGTANTAGG
TNNCTCGTATAANNCNCGGNTTGNCCNCTNTATTTNNTNNATATCNTTCCTTGACGTCNCNTNNGANC
NNNGCTNGAAGGTCNTNGANNAANTNTCCNATNACTAGCNANGTCTCCGATGATGACCNGCTNTCTTN
NAGCNCNACTNCCGNGNACCT
GCCCTATANGGAGNTGNATNACANGGGGCGGCCGCTCTAGAACTAGTGGATCCCCCGGGCTGCAGGG
CAGGGCTGAGGCAACGGTGCCCACCCTCCGGCTCTCTCTCTGCTTTTCTGCGCCTCCGCTCCGCTCTCC
GCCCGCCGCCATGACGGAACAGGCCATCTCCTTCGCCAAGGATTTCCTGGCCGGGGGCATCGCCGCCG
CCATCTCCAAGACTGCCGTTGCCCCGATCGAGCGCGTCAAGCTCCTGCTGCAGGTGCAGCACGCAAGC
AAGCAGATCGCGGCCGACAAGCAGTACAAGGGCATCGTGGACTGCATCGTGCGTATCCCCAAGGAGC
AGGGCGTGCTGTCCTTCTGGCGGGGCAACCTGGCCAACGTCATCCGCTATTTNCCCACGCAAGCGCTC
AACTTCGCTTTCAAAGACAAGTACAAGCAGATCTTCCTGGGGGGCGTGGACAAGCGCACGCAGTTTCT
GGAGGTACTTTGCGGGCAACCTGGCCTCCGGCGGGGCAGGNCGGGGCCACTTCCCTGTGCTTNGTCTA
CCCGCCTGGATTTTCGCCCGAACCCGCCTNGCGGCCCGACATTGGGCAATTCGGGGCAGTGAGCGCGA
GTTNANNGGGCCNGNGAGATTGNCCTGGTGAAGAATCACCCAATTCCNACGGCATTTCNGCNGGCTG
NACCAAGGGCCTTCAACNGGNTCNGTGNCAGGGCCATCAATCATCTTNCCNNNGCCGGCCTTACTTTC
NNGNATNTACTAAAACCTCNCAAGGGTATGTCTCNNCCNAANCCCCAANGAACAACGCANNATCNTG
GGTNGANNCATTNGTATGAATCNCNNCNTANANCCGGTGACNGAGTCNGANTGNTGGGTCTTGGTNTT
TTCTACCCCNTTTTTANNANCCGGNTTCTGCTNGGGNNCATTTAATAANTTCATNNTTCNGNGGGCNCA
ANGGGAANCCNNNANTNTCTTTCTCCNTAGGGNCGCCC
TTNNAGCCTATANGGAGNNGNATNACAGGGGCGGCCGCTCTAGAACTAGTGGATCCCCCGGGCTGCA
GGAAATTCGGCACGAGGGCAACATGGGTCACCAGCAGCTCTACTGGAGCCATCCGAAAAAATTCGGC
CAGGGTTCTCGTTNTTGCCGGGCCTGNTCCANCCGGCNCGGCCNGANCCGNAANNNCGGCCTNANNA
NGNNCCGCCNGGGTTNCCNCCAGNNNNCAAAGNNNNTNGNTTNATTNAAGGACCCNNGNNGNCCCCN
NNGGNNTCNNGTTNAAGGNNGGGCCAACANCCNGNANTTNNGGGGCNGGGNTTGNANAATGGCNGT
TTTNTTTTTTAACTATACNAGGNTTTTTCTTTGGTACNGACACACACACANTGAGAGAGGNTNGCNCNT
CATATNTATAATAGATATGAGAGGGGGATATGANAAGCGCTGTGAAAAAATATATTTTTTCTATAACC
TCTTTACACANATCTTGTGANATTTATAGGCNNTATNTNANGTGNGGNNANACANAANNNTTNACAAA
NNTCTNTAATGAGACANNTTATCACTCTACATATNTCGTGAAANTTCNGNAAAAAAAANNCGNNNNC
NNCTGTCCTNNNAAACANAATATGGGCATNTGTAGAGNTCTNGNTGTTTGGNCNCANATACCCTTTTA
GNAAGAANCAAGACTGTGTTTATAATACTATAACTNTTGTGGACAANCTCANANAAAATAATTTTTCT
TGCTGAAAAAANAATAATAAGGGTGGTGGAAAAAANTATTATGTTGACGGGTTNTTGGGTTGCTTNCA
AAAAACNTNAAAAGGGTGGTGGGTGTTTTTTTTANNAGGAAGAAAAAATTTNTTACTGGCTGCTTTTT
NAAAAAAAAAAAACTGGCNCTACCTTTTTTTTTTNATNAATNAAANAAANNACCNCCNNNGTTTNTTAAA
AACNATTTTTTTTTNANAN
TTNNAGCCTATANGGAGNNGNATNACAGGGGCGGCCGCTCTAGAACTAGTGGATCCCCCGGGCTGCA
GGAAATTCGGCACGAGGGCAACATGGGTCACCAGCAGCTCTACTGGAGCCATCCGAAAAAATTCGGC
CAGGGTTCTCGTTNTTGCCGGGCCTGNTCCANCCGGCNCGGCCNGANCCGNAANNNCGGCCTNANNA
NGNNCCGCCNGGGTTNCCNCCAGNNNNCAAAGNNNNTNGNTTNATTNAAGGACCCNNGNNGNCCCCN
NNGGNNTCNNGTTNAAGGNNGGGCCAACANCCNGNANTTNNGGGGCNGGGNTTGNANAATGGCNGT
TTTNTTTTTTAACTATACNAGGNTTTTTCTTTGGTACNGACACACACACANTGAGAGAGGNTNGCNCNT
CATATNTATAATAGATATGAGAGGGGGATATGANAAGCGCTGTGAAAAAATATATTTTTTTCTATCC
TCTTTACACANATCTTGTGANATTTATAGGCNNTATNTNANGTGNGGNNANACANAANNNTTNACAAA
NNTCTNTAATGAGACANNTTATCACTCTACATATNTCGTGAAANTTCNGNAAAAAAAANNCGNNNNC
NNCTGTCCTNNNAAACANAATATGGGCATNTGTAGAGNTCTNGNTGTTTGGNCNCANATACCCTTTTA
GNAAGAANCAAGACTGTGTTTATAATACTATAACTNTTGTGGACAANCTCANANAAAATAATTTTTCT
TGCTGAAAAAANAATAATAAGGGTGGTGGAAAAAANTATTATGTTGACGGGTTNTTGGGTTGCTTNCA
AAAAACNTNAAAAGGGTGGTGGGTGTTTTTTTTANNAGGAAGAAAAAATTTNTTACTGGCTGCTTTTT
NAAAAAAAAAAAACTGGCNCTACCTTTTTTTTTTNATNAATNAANAAANNCCNCCNNNGTTTNTTAAA
AACNATTTTTTTTTNANAN
AAAAAGCTGGAGCTCCACCGCGGNGGCGGCCGCTCTAGAACTAGTGGATCCCCCGGGCTGCAGGGGA
GGAGGCGTTGGAGTCACGGGTCAGCGGGAGCCAGCAAGGAGGGGGACAGCCACGGAGAGCTGAGGA
TGAGGATTCGGCCCCGGGCGCTCAGGCTCTCAGGACGNGCCTGTCCCAGGCCCAGCCTNTGAGCTCCA
GCAAAGCCCCGACCCGNTGCGGCCNTATTGTCTACTCTGCANACTGTCCGACCCAGGCTNGGNGCTGT
GCCTGCAGGAGGGCGGCCCCCGGNATCGNTGANCCACCATNTTAGGACTTGAGAGGCCCGCCTGGGG
ACCACGAGACNAACCCCCCAGGCTCAGCTCTGGATTCACCGAGGCCCACTCTTGGGGTCGTCTGCTGC
TTGGGTCCTTTTCGGGACCCCAACCCTTCCAGGCCCTTTTTTGCACACTTTTTCCCCCTTCTTGCCCATN
TTCCCCGNANTGCCGTGCCAGGCCTGAAGGTGGTGGGNTGGTTAAAGGCTNANCCATTACCATTTCAC
ACCTGTCCCAAAATNTGATACCCCTGCAAAGGGAAGGGACCACNCTGTTTTCCNAATGAAAGTNTTTN
CCAAAAAANACCTGGGTTTNGGNAANAATCCTTTTTCTTTGCTTTCAATTTTTCCNAAGGNAAAACCCC
AATCCTGAAAATNTTTACCCTNGGGGAGGGAAAAAAACCCTTGAATTCCNAAAAAAACAAACCCTTA
AAAGGGGTTTGGGAGGANGGGANCCTTGGGGCCCTGGNAAAGGCCCCNTTGGGGGCCNTTTGTTTTTC
TTTCTNGGGAGGGAAACCCGGGNTTTTCCCCAAGGGGGGGTTTGGGCCCNTTTTGNAAAATTNNAAAA
ANTTNGGGGCCTNGTTTNCATTGGNAAAAAAAAAAGNTTTTTTCCCCNGNAANGGGTTTAAAAAGGA
AATTTTTTTNCCCCCCCNAAAAAAAANGGG
TTNANCAAANCTGGAGCTCCACCGCGGTGGCGGCCGCTCTAGAACTAGTGGATCCCCCGGGCTGCAGG
AATTCGGCACGAGGGTCAAGCACACATTTTANGAGAAGCTNCCTGCTCATCTTGTGAAGGTTAGTGCT
GACCACAAGGAGCAGATGGCTCCATTACTGATGTTAGTGCTTTTCCAGATATGAGAAGATGCAAGAAT
TAGGCTCATAAAATCTCCTGAAAATATCCACCTGGTGGCGTGTTCTTCCAGGTTTTCCCAGAGCACAGA
GTACCTCGTTCTCGATCTCCACCTTGAACTCCTTTCAGGGTGTGTTAAGGTCAGTGCCTGCAGTGGCTA
GTGACTTAATCCTTGTAGAGGCAGAGGGCGACTGACACTTTTTAGCTGGCAATGGCTGGGATTACACG
GCAGAGTCCAAATTCAGCTGGAGGCCCAGGACCCCCGAGCTTCTCCTCAGAGGACGGTCAGGGCCTCA
GCTGCCAGTGGGGTGGCGACCCTGCCTGTCAGTGGGAAGGGCTGGGTTCTGGGTTCCCCTCTGAAAAG
TGGGGTTCTAGCCAGGGAGGCACCCCGTTAGCCCCCTTGCACCAAGGGTAGGAAGGANGTCCCTGGG
ANGGACCCTNTGNCTGCACATCAAGNTTTACCGNGACTTTTACTGGAGTCGGCAACACCGTAAATTAA
ACACGTGCTTNTTTCTNCAATCCANGGTCTTGGGCAGGTGAAGGAAATAGGCCATTTTAAAAAAATGG
TTTGNAAGGGACTTTCCCTGGGGGGGCCCAATGGGTTAAGGAACTTTCNCCCTTTCCAATGCCAGGGG
GGGGGCAAGGTTTTGAATCCCCTTGGNCCAGGGGAACTTAAAAATCCCCCCATGGCCTTTGNGGGGCC
AAAAAAGGCCCAAATTCTTTNCCTTTCCNNAAAGCCCNCCNTTNNTNAACCAAAAGGCCAATNAAAAN
AACTTTTTTNAAAGGNAAAAAAAAAAAAT
TTNNNCACAAGCTGGAGCTCCACCGCGGGTGGCGGCCGCTCTAGAACTAGTGGATCCCCCGGGCTGCA
GGAATTCGGCACGAGGNTCAATCGCANGTTTTNANAGAANCTGGCTGCNTNTGCAGCNNCTNNNACC
GNTNGACCCAGGNANCNTNNNTNTNNCTNACTGNTNNNANGNCNATTCCATNGGCCATATNTCNCCNT
ACTNTCGCACNTTTTTTNNGCCNACACANTGNNACAGNTGNCTGNTNANTCTNGGTCCTGGAGGANNT
GANATTANTAGNANCCTATATAANCCCTTGTACTCCTTTAAGGACCNTNGCNGNAATTGNCTGCNCTN
ANAAAACACTGNNNNNTNGATNATGATTNNGGNGATNTGANTCTTCCATAATAGNCAATGGTTGAAN
AACACGCTTTATGANCTTATCAGNCCGTNGGCNTGCAACNCNATNNTTTTTCATTTTAGGACCNTCTAA
GTCTTNANTTGCNNATGCTGGGAAANNCATNCANNCANGANNCAAGGGTCNGGGANAGCTTTCCCCT
TTGANAGATGGGATTGAAGNAAAGAAAAATCNCCTNGTTCTTNTGTAGCTCCAANGTGAGGAATGGA
AAAAACGTGNTNGGCTTCGTATGCCATGCGCACCCNNATTTNCTTTATTNTGNACTGGTTCNTTTACCT
CTGAAAATACCCACAGGACGTGGATAANTTNTTGGCCTTTTACCCNGGGAGGAAAAGGGCCAANAAA
CAATTCAAAACCTTNNCCTTAGGAAGGTCCCTTAANTNGNNGGCTTGCCATGGGGTNTNNAAAATTTA
NCCTTGGCCNCCNNNGGGGGTTGCCAATTNGGGAAAACTTTGCCTTTTTGGGNTTNAAAAAAACNGTT
TTTTNCCCTTTTTGGGGNNNNNNCCCNNAAAANTTTTTNTTNAGGGGGGGGCNCTTTTTTTNAANNNNC
CCCTNNNNNNNNNNNNTTAACAGTTAANGGNANNTTTTN
TTTGAACAACAGCTGGAGCTCCACCGCGGGGGGCGGCCCGCTCTAGAACTAGTGGATCCCCCGGGCTG
CAGGTTCCTGTTAACAGGNNGNTNNANGAGNCATGCAAAACNTCNACACCCCATAGGGNATGCNNNN
NAAGNTATNAANACTAAGAGGAGCTGGCATCANGCACACACNCTGTANCTCACGACNCNTTGCTTAA
CCACACCCNCACGGGAAACANCAGTGACNAAAATTATAGCCATATACGAAAGTTTGACTAAGTTATAT
TANTTAGGGTTAGGTAATATNTNGTGCCAGCCANCTGCGGNCATACGATTAACCCCNGCTAACANGGA
GTACNGCCGTAAANCNGTGTTNNAGCACCATACCAAATAGGGTTAAATTCTAACTAAGCTGTNANAAG
CCATGATAACNANATNANTTANTGACNANANGNGACNTTACANTTNCCNGACAGCACTATATNTAAG
ACCCANAACTGGNGATTAGNTCTCCATTNTGCCTAANCCTAANNACATTTTATTACATANCACAACAA
TTTNTTCNTCAGAANTNCTACTATCAACNNNCTTTATAAACNTTANAGGACTTTTGGNGNNNGCTTTTA
TATTNCATTGTTNNAAGGANGCCTGNNTNTATTNATTCNGANTNAAANCACNCGATCNAAANCTTTNC
CCAATTCNTTNCCTANTNTCNNCNTTNTNAAACNCGGCCATNTTTCATCTAANCCTCTAANTTGATAAA
CAAAAAANNNATAATTNNNNGTGGGNGGGGNCNCNCNNGATCCNCNAATTCTNNNGCTCTNTTNGNN
NAGTCNATTTTANAANACGTTTCCNANNNGTNTCANGNTTTTNNTCNNCTCCNCNANCNCTNTTNNCN
CTNTATNTANANGNTCTTGNTNNTANNTNNNTTCNTCANTANTATGTNNCNANTNATACTNTATNTAC
CNNCTGAGTNGAGATNCTACNCTGTNCNTAGTATGNTACGNAGCGCNATTTNTACATNTTCNTAGTNN
TGNTGNTNNNTNCNNTCANNATNGCNNCNGTTGATNCG
CCTATCTGGAGCTNCACCGCNGTGGCGGCCGCTCTAGAACTAGTGGATCCCCCGGGCTGCAGGCGTGA
AGGTTGGAGTCTCTCCAGAGAAGCTCCGCTTCCGACAGCACATGGAGAATGAGATGGCCCATTTATGCC
TGTGACTGTTGGGATGCTGAATCCAAAACATCCTATGGCTGGATCGAGATTGTTTGGATGTGCTGACCG
TTCCTGTTATGACCTGTCTTGTCATGCGAGGGCCACCAAAGTCCCACTCGTAGCCGAGAAACCTTTGAA
AGAGCCCAAAACAGTCAATGTTGTTCAGTTTGAACCCAATAAGGGAGCAATTGGTAAGGCGTACAAG
AAGGATGCAAAGCTGGTGATGGAGTACCTCGCCATTTGTGATGAGTGTTACATCACAGAAATGGAGAA
ACTACTGAATGAAAAAGGGGAATTTACTGTTGAAACTGAAGGGAAGACGTTTCAGTTGACAAAAGAT
ATGGTCAATGTGAAGAGATTCCAGAAAACACTACATGTGGAAGAAGTTTATTCCAAGTGTAATCGAACC
CTCTTTTGGCCTGGGCAGGATCATGTATACGGTATTTGAACACACATTCCAAGTTCGTGAGGGAGATG
AGCAGAGGACGTTCTTCAGTTTCCCTGCCGTAGTAGCTCCATTCAAATGTTCCGTCCTTCCATTGAGCC
AGAACCAGGAGTTCATGCCATTCGTCAAGGAATTATCGGAGGCCTTGACCAGGCACGGCATCTTTTAC
AAAGTANACGATTCCTCAGGATCCATTGGAAGACGCTTCGCCCAGGACTGGATGAAAATGGTGTGGCT
TTTCGCATACCCTTGGACTTCGATCNAGNGAACCAANACCCTCACACGGGCCCTTTTGCGGGGACCGA
GACTTCCTGCCGGAAAATCANAACCANAAGTCTNTTGAACCTGGCCCATGTTNTCCGCGATTTGGNCA
ATGGCAACNNCNTTTGGGCGGATNTGGAGGCCCAANN
NCTATCTGGAGCTCCACCNCNGTGGCGGCCCGCTCTAGAACTAGTGGATCCCCCGGCCTGNAGGTTNA
GAAGNGNCCATCTCCCAGACGGCCCTGCANCANAAGACCCTGAGGGTGGNCCTGTGCTCCGTCGGGA
ANCACCGCAGGNAGGANTGCCTGGNGGAAACTNAGATCAGCCTGGCGGACCTACCCTTTTCCAGNGG
GGTTTTCANTCTGGGGTATACNTNGCTTCTTTCAAAGCATTGCCTTTGCNAAAAAATAANAGAGGACN
TGGGNANTCGGTTTCAAACCANCCACCCTTTGGNNTCCCANAAANTNGTTCAGTGACCCNTTTNTNGA
AAAAACTTTAATGTANTNTTTGGCCGGGAAAANAAATTTCCCCNAAAAAANAACCCGGNGGGAAAAAA
AANACACANGNCCCAANNGGANTAGTTNNGCCCTCTAAAAGGCCGTNAAAAAATTTTGGCCCNAAAG
NGGCTCNAAAAAAATTAGTGGNNGGGNGNACTNTTTTGGGNAAAAAGACANTCTTCNCAAGTNTGCG
AAAANAAGNGGGGCCCCACCAGGAAAAAAAACNGTTNGTGCNAAAAACTNTNNTAACACCCCCCCCC
CCAGNAAAAATNCTCTTTNTTGGTNTAAAAANAAACCCCCCCCNNNCCCGGCGGGGGNNCCTTTTTTT
TTTNCCCCCNAAAAAAACAAAANNTTNNTTGGGCGCCCNNCCNCCNTTTTTTTAANAAAAAAANAAAN
TTGGNNCNTTCCAAAAAAATNTTNCTCCCNGGGGGGGGGANNCCCANNNCNNNNNGGGGGGNANNN
NTNAAANNGGNNGGGGNNNGGNNNNCCCCNCNNNTTNNAAAAAAANNTTTTNTNGGGGGGNAANNN
NNNNNNNCNNNNCNNNAANNNAAAAAANNGGGGGGGGGNCCCCCCCCCCCNNNNNGNNNNNNNNN
NNNNNNTNNNNNNNNNNNNNNNNNNNNNNNCCNNGGGNNNNNNNNNNNC
GCNCTATCTGGAGCTNCACCGCNGTGGCGGCCGCTCTAGAAGTAGTGGATCCCCCGGGCTGCAGGGTC
GTCTTGGAGGTGACTCGGCGTGATTGAATTTGCGGCATCTTCGCATTCACTCACAGGTCAAAATGCAG
ATCTTCGTGAAAACCCTGACCGGCAAGACCATCACCGTGGAGGTGGAGCCCAGTGACACCATCGAGA
ACGTGAAGGCCAAGATCCAGGATAAGGAAGGCATTCCCCCTGACCAGCAGAGGCTCATCTTTGCCGGC
AAGCAGCTGGAAGATGGCCGCACTCTTTCTGATTACAACATCCANAAAGAGTCGACCCTGCACCTGGT
CCTNCGTCTGAGGGGTGGTATGCANATTTTCGTGAAGACCCTGACCGGCAAGACCATCACCCTGGAAG
TGGAGCCCAGTGACACCATCGAGAACGTGAAGGCCAAGATCCAGGATAAGGANGGCATTCCCCCCGA
CCAGCANAGGCTCATCTTTGCCGGCNAGCAGCTGGAAGATGGCCGCACTCTTTCTGATTACAACATCC
AGAAAGAGTCGACCCTGCACCTGGTCCTCCGTCTGAGGGGTGGNATTGCANAATCTTTCGTGAAGACC
CTGAACCGGGGAAGACCATNACCCTGGTATGTGGAGCCCANNTGACACCATCNAATANCGTGGANGG
CCAANATCCAGGATAAGGGANGGCATTTCCCCCTGNACCAANCANANNNCTTAAACTTTTNNCCCGGC
CAAGCAGCCTNGGAANANNGCCCCGCANCTCTTTTTNTGGTTTNCCNACANTTCCANGAAAGAAGTCC
GACCCCTTNNCNACCCTGNGTCTTNCNTNCTNNAGGGGGGGGNNTNCCATNATNCTTNCTNGNAATGA
CCCTTGNCCGGGGNANAAANCATTCTCCCCNTNGNANGTGGGANNCCCANTGGATCNCCCTCTCNNNN
AACGTNNNANGGCCCCNTAATCCCCNGNANN
ANCAANNCTGGAGCTCCACCGCGGTGGCGGCCGCTCTAGAACTAAGTGGATCCCCCGGGCTGCAGGG
TTTTTTTTTTTTTTTTTTCCCNNNGGNNNNNCNAANGGGNCNNTNTTNGGTTTTTTTTNNNAAAAAAAN
NNTTTTTCNNGGGGGGCNTTNNNNNGGGNGGGNAACCNNGNCTTTNNNNTTTCCGGGAAGGGGNGGN
AANNNCNTANNTTNNCCCNGGCNNNAAANTNGCCCTTTTNANCNNCCCCCCNNNTTTTTTTTTNATTTT
GGCTTTTTNNGGATAAANAAAACCCCCTNTTNNCGGGGGGNNNGNTTTTTTTTNCCANNANGGNCNTN
NTTTGGCCCNTANNGGGNNGGCCCCCCNTNCAGGGNTTTTTTTNGGGGGGGCNCCCNNCCCNNTTTTC
CNCNNTAAAANCCNNGCACCGATTTTTTNAAGGGGNAAAAGGGNTNTNAACTTTCCNCCCNTTTNNNG
GGNTTTTNNGGGGGGNNGNGAAAAAACGCCCNCNNTTTTTTTTTTNNNGGGCCCCNNTCCCNGGNNNA
AAAANGNTNTTTTCTGNGNCAAGACCNGGGGGGAAAACCCCCNCNTTNACAAAAAAATTTTTTNGGC
CCCCCNCNGGGCNCNTTTTTTTGNNAAACCCCTCCNAATTTTTNNGGGGGGGGGNGGGGCCCCANTTN
NCCNNTAAAANGGGNGGGGGNNAANACNNNNNNNNNAAAANNNNNAACTTNGNNCNNNNNCCNNN
NNNCNCCNTTNCCANNNNANNCCNNANCCNNANNGGNNNNNCCNCCCNGGCCCNGGGNNNCNNNAN
NNNCCNNCCNGCNNGGCCNCNNTTNCCCNNGGGGAGGNCCNNNANNNNNNNNNCNNGGANNNNCGC
CCCNCNCNNCNANNANGGGNGCCNGNNCNNGGNAANCCCGGNNNNNNGGNCCCCNNNCN
ANCAANNCTGGAGCTCCACCGCGGTGGCGGCCGCTCTAGAACTAAGTGGATCCCCCGGGCTGCAGGG
TTTTTTTTTTTTTTTTTTCCCNNNGGNNNNNCNAANGGGNCNNTNTTNGGTTTTTTTTNNNAAAAAAAN
NNTTTTTCNNGGGGGGCNTTNNNNNGGGNGGGNAACCNNGNCTTTNNNNTTTCCGGGAAGGGGNGGN
AANNNCNTANNTTNNCCCNGGCNNNAAANTNGCCCTTTTNANCNNCCCCCCNNNTTTTTTTTTNATTTT
GGCTTTTTNNGGATAAANAAAACCCCCTNTTNNCGGGGGGNNNGNTTTTTTTTNCCANNANGGNCNTN
NTTTGGCCCNTANNGGGNNGGCCCCCCNTNCAGGGNTTTTTTTNGGGGGGGCNCCCNNCCCNNTTTTC
CNCNNTAAAANCCNNGCACCGATTTTTTNAAGGGGNAAAAGGGNTNTNAACTTTCCNCCCNTTTNNNG
GGNTTTTNNGGGGGGNNGNGAAAAAACGCCCNCNNTTTTTTTTTTNNNGGGCCCCNNTCCCNGGNNNA
AAAANGNTNTTTTCTGNGNCAAGACCNGGGGGGAAAACCCCCNCNTTTNACAAAAAAATTTTTTNGGC
CCCCCNCNGGGCNCNTTTTTTTGNNAAACCCCTCCNAATTTTTNNGGGGGGGGGNGGGGCCCCANTTN
NCCNNTAAAANGGGNGGGGGNNAANACNNNNNNNNNAAAANNNNNAACTTNGNNCNNNNNCCNNN
NNNCNCGNTTNCCANNNNANNCCNNANCCNNANNGGNNNNNCCNCCCNGGCCGNGGGNNNGNNNAN
NNNCCNNCCNGCNNGGCCNCNNTTNCCCNNGGGGAGGNCCNNNANNNNNNNNNCNNGGANNNNCCC
CCCNCNCNNCNANNANGGGNGCCNGNNCNNGGNAANCCCGGNNNNNNGGNCCCCNNNCN
AAAAAGCTGGAGCTCCACCGCGGTGGCGGCCGCTCTAGAACTAGTGGATCCCCCGGGCTGCAGGANTT
CGGCACGAGGAACAACGCCNTGNTNNTNNTNNNGAANNGGANCNGGAAAGCCCNNNNCGNATNCGA
NNTNANGCTGANCGGGACCGACTCACAGNNNNNACNAANGCAGCCTGATGTNACACATNGNNACCCA
TNATNNTCTACTGTCNCAATCNACATGTACCANNANGCATGNGCCNAGCNGTNTANACCTTGATCAAG
TTAATGCTATTGANGCTANAGGNTACACNTTNTCANANCCTGANCNAGGGCCTTGGGAAGNANNTGC
GNAANCTNCNAANNGCCATTNNTTCCNCGCCGCNGATACAGGGCGAGTACATGATCCNGTGCGAGAA
GGNNNCCANCCTGCCTTANGTNACCGTGAAACTGGGGGGTAAGGACTACGCGCTGTCCCCANAGGAC
TACGCGCTCAAGGTGTCGCAAGCCGGCACGACCGTGTGCCTGANCGGCTTCATGGGCATGGACATTCC
CCCGNCTGGCGGGCCGGTTTTGGATNCTGGGCGACGTCTTTATNGGGCGTTACTACACCGTGTTCGAAC
GGGAACANAAACCGGGTGGGCTTTGGNCCAAGGCTGGCCCGGTTTTAACTTGGCCCCCGCTTACCTGG
TTGGANTNCAAAAGAAGAAGGCGGCCCAACAAAAAAAACCAAAAAAAGNANAAAAAANTTTTTGCTT
AAAATTTGACTTTCNAGNTNGCAACCAAAANNCCCGATTATTTCCCAAGGGCCCNTNGGGNCCNNAAT
TTNGGGTTTCCGGGGGTTTCCCCCTTNCCTTGGGGGCNNNAAAAAGCCNGNNTTGCNNGGGNCNNGGC
TGGGCNTGGNCNGGNNNGAAAGGGGGAAGGGNAANCCTTGGAACCCCAAANTTGGGGGGGNAAANT
TTCCGGGGGGCNCAAAAAANCCCNTTTTTTTTTAAATTTGGNAAAAAACN
TTNAACAAAAGCTGGACTCCACCGCGGTGGCGGCCGCTCTAGAACTAGTGGATCCCCCGGGCTGCAGG
AATTCGGCACGAGGGTGCTCGTCCACTGATATCAGNGNACTCCGAAAAGGGGGAGTCCTCTGGCAAA
AATGTCACTTTGCCTGCTGNGTTCAAGGCTCCCATTCGACCCGATATTGNTAACTTTGTTCACACCAAC
TTGCGCAAAAACAACANACAGCCCTATGCTGTCAGNGAATTANCAGGTCATNAAACCANTGCTGANTC
TTGGGGTACCGACAGAGCTGTGGCTCGAATTCCCAGGGTTCGAGGTGGCGGGACTCACCGTTCCGGCC
AGGGCGCTTTTGGAAACATGTGTCGTGGGGGCCGCATGTTTGCGCCAACTAAGACCTGGCGACNTTGG
CACCGCAGAGTGAATACGACGCANAAGCGATACGCCATCTGCTCTGCACTGGCTGCCTNAGCCTTACC
AAGCGCTGGTCATGTCTAAANGGTCATCGTATAGAGGAAGTTCCTGAACTTNCTTTGTGGGNGGAAGA
TAAAGTNGAANGGCTACAAAAANACCAANGAGGCTTGTGTTGCTTCTGAAGAAACTTAAGGNCTGGA
ATGATATCNNAAAAGGTCTATGCCTCTCANCGAATGAAANCTGGCAAAGGCAAGAATNAAAAACCCG
NGGCCGTATCCAGCGCANGGGACCCTTGCNTCNTATATAATGGNGGACAATGGNTTCNTTCANGGCCT
TCAAAAACATTCCTGGAATACCCTGNTAAAGNAANCAACTTNACANTTTGNAACCTGCTTCCTGGGGG
CACCNGGGAACTTTTTTCTTTTGAATGAAAAGGCCTTTCCGAAAGTNAAAAAAACTATTTGGGCCTTG
GGNGAAAANNNNCCTCCTTTAAAAAAAACNTTNAACCTTNCTTTTNCCNAAAAA
TNNNNNAAAGCTGGAGCTCCACCGCGGTGGCGGCCGCTCTAGAACTAGTGGATGCCCCGGGCTGCAG
GCCGCCATGGGCCGCGTGATTCGTGGGCAGAGGAAGGGCGCCGGCTCCGTGTTCCGCGCACATGTGAA
GCACAGAAAAGGCGCCGCGCGCCTACGCGCCGTGGATTTCGCCGAGCGACACGGATATATCAAGGGC
ATCGTGAAGGACATCATCCACGACCCGGGCCGCGGAGCGCCCCTTGCCAAAGTGGTTTTCCGGGATCC
GTACCGTTTTAAGAAGCGGACAGAGCTGTTCATCGCTGCTGAGGGCATCCACACCGGCCAGTTTGTGT
ACTGCGGCAAGAAGGCCCAGCTCAACATCGGCAACGTGCTCCCGGTGGGCACCATGCGTGAGGGCAC
CATCGTGTGTTGTCTGGAGGAGAAGCCTGGTGACCGAGGCAAGCTGGCAAGAGCCTCTGGAAACTATG
CCACAGTCATCTCCCACAACCCTGAGACAAAGAAGACGCGAGTGAAGCTGCCTTCGGGCTCCAAAAA
GGTGATCTCCTCTGCCAACAGAGCTGTGGTCGGTGTGGTGGCTTGGAGGTGGCCGCATTGACAAGCCC
ATTCTGAAGGCCGGCCGTGCCTACCACAAGTATAAGGCAAAGAAGGAACTGGTTGGCCCCGGGTGGC
CGGGGTGTGGCCATGAACCCTGTCGAGCATTCCTTTTGGANGNGGCAACCACCAACACATTNGGCAAA
CCCTTTTATTNTTTGCAANAAAACCCCCCTTGCTGGGCCCCGGAAANGGGGTTTTTATTTGTTGNCCCC
CCCGGAAAAGGCCCTTTTTCNGGGGAAACCCAAAAANTGGGCCNGGAAAAAAGGAAAACCTTGGGGT
TGGGGCTTNAAAAAAAAAGTTTGGTTTTTTTTCCCACCTGGGAAAAAAAAAAAAAAAAAAAAANCTT
GGGGGGGGGGGGCCCGGNCCCCCCTTTT
TNNNNNAAAGCTGGAGCTCCACCGCGGTGGCGGCCGCTCTAGAACTAGTGGATCCCCCGGGCTGCAG
GCCGCCATGGGCCGCGTGATTCGTGGGCAGAGGAAGGGCGCCGGCTCCGTGTTCCGCGCACATGTGAA
GCACAGAAAAGGCGCCGCGCGCCTACGCGCCGTGGATTTCGCCGAGCGACACGGATATATCAAGGGC
ATCGTGAAGGACATCATCCACGACCCGGGCGGCGGAGCGCCCCTTGCCAAAGTGGTTTTCCGGGATCC
GTACCGTTTTAAGAAGCGGACAGAGCTGTTCATCGCTGCTGAGGGCATCCACAGCGGCCAGTTTGTGT
ACTGCGGCAAGAAGGCCCAGCTCAACATCGGCAACGTGCTCCCGGTGGGCACCATGCCTGAGGGCAC
CATCGTGTGTTGTCTGGAGGAGAAGCGTGGTGACCGAGGCAAGCTGGCAAGAGCCTCTGGAAACTATG
CCACAGTCATCTCCCACAACCCTGAGACAAAGAAGACGCGAGTGAAGCTGCCTTCGGGCTCCAAAAA
GGTCATCTCCTCTGCCAACAGAGCTGTGGTCGGTGTGGTGGCTTGGAGGTGGCCGCATTGACAAGCCC
ATTCTGAAGGCCGGCCGTGCCTACCACAAGTATAAGGCAAAGAAGGAACTGGTTGGCCCCGGGTGGC
CGGGGTGTGGCCATGAACCCTGTCGAGCATTCCTTTTGGANGNGGCAACCACCAACAGATTNGGCAAA
CCCTTTTATTNTTTGCAANAAAACCCCCCTTGCTGGGCCCCGGAAANGGGGTTTTTATTTGTTGNCCCC
CCCGGAAAAGGCCCTTTTTCNGGGGAAACCCAAAAANTGGGCCNGGAAAAAAGGAAAACCTTGGGGT
TGGGGCTTNAAAAAAAAAGTTTTGGTTTTTTTTCCCACCTGGGAAAAAAAAAAAAAAAAAAAAANCTT
GGGGGGGGGGGGCCCGGNCCCCCCTTTT
CNNNANCTGGAGCTCCACCGCGGTGGCGGCCGCTCTAGAACTAGTGGATCCCCCGGGCTGCAGGAATT
CGGCACGAGGANGACATNNNACTGCNNNNTTATGNGTGCNTTCCTGGCTCCTGCCCTNGGGNANGNT
GCGAATGCCNTGNANCANCTGGANCGNNATGACCGTGANCTTGGGCGTGGAGGACCATATNACACCA
TCNTANACNTNTATGGNGGAAGGCATGCACAAANNNNNCTGGCCGACNAGGACCAGCAACAGGCTGA
NCTTTNCCGNCACACAGGGGCGTGATACTNNCTATCTGTNANACTACACCCTACGANATGAGGTGGCT
CTGATCTTGAATCTGNTGCTGNACATNNGGCNGTGTTNGNTTNTTATTTNCCAANCTCTATNCANNGNC
CTNGAACTTNCTTTTCACTNCATATGTTGCTTTCGATNTAATNTTGACAGCCAGTACNGGGGNCCCCGN
NCCCGTTATNTATNTCCAAAANNCTTGGGAAAAAAGNGTAATTNGACCAAAAAAGGATATTTGTTTTT
GCTATTACACCAAATACAGTTCANATGCTTATTGTTCTATTTTTTTACCAATTTCAATTTCAAAATGTCT
CANTGGTGCTATAATAAATAAACTTCANCACTCTTCCAATAACACTGCGTTACATTCTTTGAATCCTAN
CCCATTTTCAGAGCAATTGACGGTGCTTACCATTTAAAAAGATTACCTTNCTTTTTGAAACAGTCAAGCA
AGAAATTAGAAAAGAACTTCTGTTTCAACTACCTTAACCTGGGCAGAAACATAANGAATTCTATGAGN
CCAAAAAAAGAAAAATTTGNCCNAAANGGCCAAAACTTACGNTCCTTAAATATGGGTTTAAAAAAAA
NTNGAACGGNGGGCAAGAAATTAAAANAAATANTTTTNGANNNNCCCNTTATATCTTGAAANTCAAC
TGGTTTTAAAGGGGCTTTTNTTTTTTNNGGNCATTGGNGGGCAN
GCCCTATAGTGAGTCGTATTACAGTGGCGGCCGCTCTAGAACTAGTGGGATCCCCCGGGCTTGCAGGN
NTNAGAACTCAGNCCCCACANTGNNCCNNCGGTTACTTACTGGCTTGNCCCTAACCNNGGATGGAACT
NTGGATGCTNTCNNAGCANACTGGGGATTTCTNTACTGNCNAANACCTTGCATTCCGGGGCTCAAANC
TNAAAAACCTCCGATNCCAAAGACCTGGTNCCANAATGTCCANGGCCAAAGAACCAATGTNTGGNNN
NGAGAANCTNTCANCNGTGGNACCCAGTTNNAATTTAATGTTNANGGATTNACCACCANGGAAATGN
NTACCCAACTTGCCTTCATGCGNNNGTTGGCCAACCATGCCTATTNCANCATCACCTACCATTGGGAA
GAACACNATTGCTTACNTGGATGAGGAAACTGGCAACCTGAAAAAGGCTGTGATTTCTGCAAGGATCC
AATGATGTCNAACTTGTTGTGCGAGGNGCAACAANCAAGATTCACTTACACTGTTCTTGTAATANGGC
TGCTCTAAAAAGACTACTGAATGGCNTNAGACAATTNNTTGNATTTAAAACAAACANGCCATNTCGCC
TNNCTATCCTTGAAATTTGCACCTTTNGGACCCTCGGGTGGGCGCTTGACCAAGGAAATCAGATTTAA
CCTTTTGGCCCCGGTNTGTTTTCCAATAAAACTAAACCTTNTGNCCTTAANTTTGANAAGNANAAAAG
GGGAAAATNTTGGAACCANTTTCTTNTTGGGCCCTTNNNGATTTTTTGTTNCTTTTCCNAACCTGGAAA
ATGNTGAANANCCTTTCCCTTTTNTTTTTGGCNCCANTTTTTTTTTGNTTAAAAANTGGTGGGGCCCC
NAAANAAGNGGGAAANGGCTTTGNTTCNAAAANCTTTTGTGGGCNTTAANCAATTTTAATTTTCNCCTC
CCNCCCTCCTTTTNANCCNCNTTACAAAAAAAATTTGGGANTTTTTTTTTTTTTTNAACATTTTTNCTT
TNTTTNGGGNAAAAAGGTCNCNNTTTTNGNAAAANATCCCNAAAAAANTTNTGTAAAANACTACCCC
CNNCT
AAAAGCTGGAGCTCCACCGCGGTGGCGGCCGCTCTAGAACTAGTGGATCCCCCGGGCTGCAGGCGCA
GGATCAAGGTGAGAAGGAGAACCCCATGCGGGAACTTCGCATCCGCAAGCTCTGCCTCAACATCTGCG
TTGGGGAGAGCGGAGACAGGCTGACCCGGGCAGCCAAGGTGCTGGAGCAGCTCACGGGCCAGACCCC
AGTGTTCTCCAAAGCTAGATACACTGTCAGGTCCTTCGGCATCAGGAGAAACGAAAAGATCGCCGTCC
ACTGCACCGTCCGTGGGGCCAAGGCGGAAGAAATCCTGGAGAAAGGNCTAAAGGNGCGAGAGTACG
AGTTAAGAAAAAATAACTTCTCAGATACTGGAAACTTTGGCTTTGGGATCCAAGAACACATCGACCTG
GGGATCAAGTATGACCCGAGCATCGGTATCTACGGCCTGGACTTCTACGTGGTGCTGGGTAGGCCAGG
TTTCAGCATCGCAGACAAGAAGCGCAGGACAGGCTGCATTGGGGCCAAACACAGAATCAGCAAAGAG
GAGGCCATGCGCTGGTTCCAGCAGAAGTATGATGGGATCATCCTTCCTGGCAAATAAATTCCCGTTTCT
ATCCAAAAGGTCAATAAAACATTTTCAATGAAATGTAAAAAAAAAAAAAAAAAAAAAAACTCGGGGG
GGGGGCCCGGGGNNCCCCAATTTNCNCCCTTTTTNNGGGGNGGGGGNTNNAANNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNTNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNC
AANACTGGAGCTCCACCGCGGTGGCGGCCGCTCTAGAACTAGTGGATCCCCCGGGCTGCAGGAAAAT
CTCACTTTGATGAAATGTATTAAGCATAAAGTACTAGTCTGTGTCTATGCATATGGGAACAGTNCT
AACCAGGAAAATNNCNCTGCNNGNCNGNCATTTTTNGTTTAAAANCNGGGNNTNGNCTNGAATAACC
ANNCCNTNAGGGNNTTTTANCCNNGTAAANNNGNNTTNAAAAAANTTTNGTNGCCNNGGANNAANGG
GCNNNAAANTTNTNNCNANCTNNTANNTNANGGCCCAGGGCCNGANCAAACCAGGGNNAACNNNNG
AGAACCGCGNTCCTNTGTACCANGGGGTGGCACANGTGTTGTNACACCCTGGGGTTTCCTTCTNTTAT
ATAAGGGAGAGAGAAAAAANATGCGCCCACACANNACAGATATNCCTTCCACGCGCGCAGTTNTTGA
TACNCCACNCNNTGTNTGNGGCNNNNACNCNNNNACNTGCCCCCTNGGGTNTAAAANAGNCNCCNNN
CGCATCTNTATTTTCTGANACTCTCNTNTGTGTNANGACANTGTTTTTNNAAGAAAATGAGGACACCCT
ATNGGTAAANCCNCTGATGAGTGTATANACCCCTCCNTGGGGGTCTCNTGNGCGCTTCAAGCGCCCCC
TTTCCCCACCNCCCCATGNACCANTGNGGGNGGNNTCTGTNAAAAAAAATCCTTNTCTCTNNCNCCCC
CCCATTTTNTNTTTTNNANAACNCCTTGGGGGCCCCCCNGCCGTGGNGGNCANAGNACTTCCCNGCNN
GGTGGNTTTTTCCCCGGGGGGGGGGCNACAACCAGCCCNNGAAANAACCCCCCNCCTTCAAAAAAGG
AGGGTGCTTTTTTNAAAAACCCCCNTNTTTGCCCCCNCNNC
AANACTGGAGCTCCACCGCGGTGGCGGCCGCTCTAGAACTAGTGGATCCCCCGGGCTGCAGGAAAAT
CTCACATTTGATGAAATGTATTAAGCATAAAGTACTAGTCTGTGTCTATGCATATGGGAAACAGTNCT
AACCAGGAAAATNNCNCTGCNNGNCNGNCATTTTTNGTTTAAAANCNGGGNNTNGNCTNGAATAACC
ANNCCNTNAGGGNNTTTTANCCNNGTAAANNNGNNTTNAAAAAANTTTNGTNGCCNNGGANNAANGG
GCNNNAAANTTNTNNCNANCTNNTANNTNANGGCCCAGGGCCNGANCAAACCAGGGNNAACNNNNG
AGAACCGCGNTCCTNTGTACCANGGGGTGGCACANGTGTTGTNACACCCTGGGGTTTCTTCTNTTAT
ATAAGGGAGAGAGAAAAAANATGCGCCCACACANNACAGATATNCCTTCCACGCGCGCAGTTNTTGA
TACNCCACNCNNTGTNTGNGGCNNNNACNCNNNNACNTGCCCCCTNGGGTNTAAAANAGNCNCCNNN
GGCATCTNTATTTTCTGANACTCTCNTNTGTGTNANGACANTGTTTTTNNAAGAAAATGAGGACACCCT
ATNGGTAAANCCNCTGATGAGTGTATANACCCCTCCNTGGGGGTCTCNTGNGCGCTTCAAGCGCCCCC
TTTCCCCACCNCCCCATGNACCANTGNGGGNGGNNTCTGTNAAAAAAAATCCTTNTCTCTNNCNCCCC
CCCATTTTNTNTTTTNNANAACNCCTTGGGGGCCCCCCNGCCGTGGNGGNCANAGNACTTCCCNGCNN
GGTGGNTTTTTCCCCGGGGGGGGGGCNACAACCAGCCCNNGAAANAACCCCCCNCCTTTCAAAAAAGG
AGGGTGCTTTTTTNAAAAACCCCCNTNTTTGCCCCCNCNNC
TNNNNNAAAAGCTGGAGCTCCACCGCGGTGGCGGCCGCTCTAGAACTAGTGGATCCCCCGGGCTGCA
GGAATTCGGCACGAGGAAGACAAACCCACACGTCCCGGTGGTGGAAACACCCCATGAGGGCGCATCC
TCTTCTCCTGGTTAGTATTTATTTCAGCACCTGGTTAATGCGGTTTTTTATTCTCTACCTATTGCACTGTT
GTGACTTGTTGGCCATTATTTGATTTTTGTACGAAAAAAAAGCTTTGTTATAGAAATCAGCATACTATT
TTTTTAAACCTGGAGAGAAGATATTATGGTGACTGAAAATATGGTCAGGTGTCAAATATAAATGTATA
AAAGCCTTCTTACTGGNCCTGGCTGGTCATGGTACAATTAATTTATATTGGCTGGCCAATACTGAAGGT
TCTTTTTCGGTTGGGGAAACTCTAATTTCTATCACNGNGGCATGAAATTTTAAAATCAGGATTTCATTA
CAGGTGGNCTCAACANTGGGAAACTTAATTTTGGCNGGANCATTATTGATCAAGCCAAATAAANTTTCA
ACAGNCCTTTGGAGGGANAAAAAAAAAAAAAAAAACTGGGGGGGGGNCCCGGNCCCCAATNNNCCC
TAAAGGGNGNGGGNNNANANNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNCNNNNNNNCNNNNNNNNNNNNNNNNN
NNNNNNCNNNNNNNNNNNNNNNNNNNNNNNNNNNNNNNNNNNNNNNNNNNNNNNNNNNNNNNNN
NNNNNNNNNNNNNNNNNNNNNCNNNNNNNNNNNNNNNNNNNNNNNNNNNNNNNNNNNNNNNNNN
NNNNNNNNNNNNNNC
TTNAANAAANCTGGACTCCCCGCGGTGGCGGCCGCTCTAGAACTAGTGGATCCCCCGGGCTGCAGGAA
TTCGGCACGAGGCNCCACCCCAGGCTTGGACAATGGGNGGGGACATGNTCAACGCCAGGTTCATTCG
AAACTTGCATGAACGTNGCAGCACCAGGCCTTGGTGACCGCAGCCCCCTCTCACACCTTCAAAATCAT
TATTATTTCTATCTCACACANGACTTAGTCAAAGCTTGCCCTCGCCGTGTATGTATTCATTCATTCATTT
ATTCATTCTACAGGCTTTTTTTTTTAAAANNANNGCCAAGTCATTNNAATNTTATTTTTAACGAAAACA
ATAAAGATTTTTNACCACGAGAAAAAAAAAAAAAAATGATTNGGGGGGGGGCCNGGGCCCCNNTTT
CCNNTNNNANNGATCAGAANNAAANGNGAGNGGNTNCCCNTTNCCNAATGNNNGCTTANAANAANA
CTTTGTAANNTNGCNCCATNNANCNTTTACANNGGTCNCTTNTTAAANNNAANNTTATGATTNNNAGN
NTTNCNCCCNNAATANAANGAATATNCCAGTCCNAAGNNANAANCTTTAAAANNAACTNTTCNNTGG
GGGCCCAGGNATANTANNGNGACNNTTTNANAGNNAAANAANTNNNTNTNNGGNCNNNCCTNGGGN
NNAACNTTNCNGNANGNTTTNNNATNGGNAANNCANNAANCCNCCTNNCTCCGTANGNNNAANNNN
NGGNNNNNNANAACANNNNNNTTTTTTNNANTCCCTNGGNNNCCCAANNNNNNNNNGGNNNNANNN
TTTNACCCCTNNNNTTAAANNNNNNTTNNANTTTNNNTNTTTNNNNNNNCCCNNNNAAAANNNNNNN
NNNNANGNGNNNANNNTNANNCNTNTNNNNNNNTNTNAANNNNNNGNNNAANNTNCNNNNNNNNN
NNNNNNANNNNNNNNNNNNNNNNNNNNNNNGGNNTTNGNANGGGNNCCCN
TNNANAAAAGCTGGAGCTCCACCGCGGTGGCGGCCGCTCTAGAACTAGTGGATCGCCCGGGCTGCAG
GAATNCNNCNCNAGGGNTNNCNGTTNNNNGCNNNTGNTTTCANCANACANATGCTATTATTNNGGT
GGGGGTNNANGCNNTNNCNNTNCTGNATTGCATGAATGCTTGNCATTGGACATTGANANACATTCTTN
AANNACTGAACCNNTGNAGGAGAAAGTNNAANNNNTNGTCACACNGNACATGNGATGACTCTANTGC
GATANCATGGGCTGNAACCTGCCAGGCTGATAANGTCCNNTGTATTNNCCAGNAAGAATACTNGGGT
GCTTTCCCTATTCCTTTGCNGANGGGACTTTANTNANCCAANGATCNAACNCNACTCNNNTGGTTGNG
GAGGGCAATNTTTTNTGNCTGACTCANNACAGAATTCNCGNCGTTACGGTGTACATTAANTGGAGNGC
ACATCTACCACTTTATGGCCGNAGAGAAATGAGCNAACNCGTCNCTGGGNAANNCAAGACTAGGGAA
CTGGATGCTACCANGAANANGGANGATCTNTACCCATTTTCCTAGTTCGANTGCAATNATANNCATT
CCACTTAGCCGGAAGACGACATANGAAGNGCTTGCGCNCNCNCCTTTTGGACCCNCGGAAACCTGNCT
TANCAGGACCTTTCCGTTGTAAACACNCCCGGNCCCGGAAACCTTTGGTTNCGTTTGACATATAGCCTT
TGGAAAAATGAAGGACTNGNTAGNANCCTGGGCNGTNAAAAANATTGAACAAGTTTNCCAANNGGGG
GACCCNCCCCCCCCGAANCTTGAANCCTTTTTTTAAAGGGGTTTTTTTCAAGAGTGGNCAAAAAAAAC
TTCAAGGGGGCAAACCCGTTTNTAANAGTTGGGTCAANCATTTTGGAANCCAAAATTTTGANTAGGGG
GANAAAANCCTCGNAAAAACGGGGGGNGNCCTTTATTGAACCCCNCCCGGNNAAAAAAAAANTNNTN
TTTTTTNAAAGGNTANTTTTNTTTTNT
TNNNNCAAANCTGGAGCTCCACCGCGGTGGCGGCCGCTCTAGAACTAGTGGATCCCCCGGGCTGCAG
GTCAAAAGACCCCTTTCATCACCAACCCTGGGTATGACACTGGAAACGGTATTCAGCTTCCCGGCACT
TCTGGTCAGCAGCCCAGTCTTGGGCAACAAATGATCTTTGAGGAGCATGGTTTTAGGCGAACCACACC
GCCACCACGGCCACCCCCGTAAGGCATAGGCCAAGACCGTATCCGCCGAATGTAAATGAGGAGATCC
AAATTGGTCATGTCCCCAGGGGAGACGTAGACCATCATCTCTACCCTCACGTTGTGGGACTCAATCCA
AATGCTTCTACAGGCCAAGAAGCTCTCTCTCAGACAACCATCTCATGGACCCCATTCCAGGAAAGCTC
TGAGTATATCATTTCATGTCATCCAGTTGGCATTGATGAAGAACCCTTACAGTTCCGAGTTCCTGGAAC
CTCTGCTAGTGCGACCTTGACGGGCCTCACCAGAGGGGCCACCTACAAGATCATAGTGGAGGGAGTAA
AAGACCAGCAGAGGCAGAAGGTTCGCGAGGAGGTGGNTACCGTGGGCAATTCTTGTTGACCAGGGCC
TGAGTCAGCCCACAGATGACTCCTGCTTCGANCCCTACACGGGCTTCCATTATGCCATTGGGAAAAGA
NTGGGAACCNATTGTCTGATTCTGGCTTTTAAACTCTCGGGCCCATGGCTTAAGGCTTTGGCAGGGGTC
ANTTTCAAAATGCCAATTCAATTTAAATGGGGGCCNTTGACAATGGGAGTGGAACTACCAAGAATTGG
AANAAAAAAGNGGGNATCCGTCANGGGAAAAAAAATGGGCCAAAAANAATNAANCTTGNCCCTTTTT
TTTGGGAAAANGGGAAAANGGGAAAAATTCAAGNGNGGAATCCTTTTTGGAAGGN
AAANCTGGACTCCACCGCGGTGGCGGCCGCTCTAGAACTAGTGGATCCCCCGGGCTGCAGGAATTCGG
CACGAGGAAGCCATGTAATGTACTATGTATTTACCCAAGACTCTCTCTTCAGGTCGGTTCCCCCTAAA
GGCTCAGAACCGACTCGACAAACCAGTATGTCTTACTCACACCTGGTTCTCCTAATCTATGTTAATGAA
ACTATATTTGCTTGGAAATCCACCTTTCTTCAAGATTCATGTCAATCATTTTATGGCCCGGATGACTCA
CCTGCTGCCAATGTTATCTCAATGCATGTTGCCGGTGAGGGGCCTGGTGCCATTCTCTGAGACGTTGTC
TTTCTTTAATTAGCAGACTGCTAGTGGCTGTAAGTGAAAGTGAAAAGTGAAAGTGAAGTCGCTCAGTC
ATGTCTGACTCTAGCGACCCCATGGACTGCAGCCCACCAAAGCTCCTCCATCCACGGGATTTTCCAGG
CAAAAGTACTGGAGTAGGGTGCCATTGCCGCAACATGCTATGGCTCTTCAGAGGAGAGACTGACTATT
TAAGGAAATCGCATGTTTAAAAGTAGTTTTCAAAAAAAAAAAAAAAAACCTCGGGGGGGGCCCGGGN
CCCATTNCNCCCTATNGGGGANGNNNNANAANNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNC
AGCCCTATAGNGAGTCGTATTACAGTGGCGGCCGCTCTAGAACTAGTGGATCCCCCGGGCCTGCAGGA
ANTTCGGCANCGAGGCTGGCTTGCAGTTTAACCTGTCCCTGTACTGGAGTCACGAGGCGCTCTTCCAG
AAAGAAAGATCCTTGCAGGGCCCTGGGAGTTCCACACCGTGCCNTTTCGAAGCTGGNTGGGNTTCAAT
ACTGGGGCCTGAACCTTCACTGAGGGCACNTACCAGCCCCGGNTTTACACCTCGCCCACCTGGAGCCA
ATCCGGGATGAAGCTCCAGTTACAACCCCTCCCGGTCCTTCCAAACCCCCCAGCACCCCAGCTTCCTTT
TCCATGACAGCTCTGTCTCCTGGNCTTTCGTCTACCTCCCTACCCTNCANAGCTGGTGGAACTACGGGT
TCTCGNGCTCCTCTGATGACCCCCCACTCCTGGCTNTTTCCAAGTNCAGCTNCTCCAAGTCCAATCCCA
CCATTGGNTATNAAAACCGGGCCCTGCTGCACTGNNAGGGGAGCTTTGGGGTAAAACGTTATCGACTT
NAAGGGGCTGGAAGGCCCTTGGTCCCCAAGTGCCCCTGGCTACCAACAGCTTCANGGAGCACTTTCNT
TTTTCCCTGCCCGGNAAGGGGGTTCTTTTNAGGAGGGTTNCAAGTCNTCATTCCGNCCCTTTTTACCTG
ACCCAACTTCNACGGAACATGGGACTAANAACTTCGGAGGGGTGGAATTTNAAGGGNNGGGGCAAGG
AANNTTNATNGGAGGGNTGGGGNACCCTNCCTTTCTTTAACCTTTTTTTTTTGGANGCACCCTTNAAN
TNACCGGGCCAACANGGGGGCCCCCCTTTGGTTTNNGNTGATTTTTNTNGAGCTANNGGAAATTTGCG
GGGAAAAAATTNCNNTNCNTNGNAANCTTTCCTNGGGGGGNTTAGGGGGNAAAAAAAANTTTTCNTN
TANAGNAAAAAAAACNNGNTTTNACCCCTGNGNNGNTNAGGGANCCCCNGGGAANAANGNAAAACC
CCCCCNTCCNCNNTTTNGGCTAGAAAAACT
NAAANNCTGGAGCTCCACCGCGGTGGCGGCCGCTCTAGAACTAGTGGATCGCCCGGGCTGCAGGCGG
CGGCGAGATGTCGGGAGGATCAGCTAGGAGCCTGGGCAAGGGAAGCGCCCCCCCGGGGCCCGTCCCC
GAGGGCCTGATCCGTGTCTACAGCATGAGGTTCTGCCCGTATGCCCAGAGGACTCGCCTGGTCCTGAC
GGCCAAGGGTATCCGGCATGAAGTCATCAACATCAACCTGAAAAATAAGCCTGAGTGGTTCTTCAAGA
AGAATCCCTCAGGCCTGGTGCCGGTTCTGGAAACCAGTCAGGGTCAATTGATCTGTGAATCTGCCATC
ACTTGTGAGTACCTGGATGAAGCATATCCAGGGAAGAAGCTGTTGCCAGGCGACCCCTATGAGAAAG
CTTGCCAAAAGATGGTCTTGGAGTCCTTTTCTAAGGTACCACCTTTGATATTGAAGATTCTTAGAACAC
AAAATAAGGAAGACTGCTCTGGCCTAAAAGAAGAATTGCATAAAGAAATCACCAAGCTAGAGGAGGT
TCTGACTGATAAGAGACAACCTTCTTTGGTGGCAATTCTCTTTCTATGATTGATTACCTCATCTGGCC
CTGGTTTGAACGTCTGGAAGCTCTGGAGTTGAATGAGTGTGTAGACCACGCTCCAACTCTTAAAGCTG
TGGATGGCAGCCCATGAAAAANGATCCCACAGTATCATCTCTTCCTCACTGACGTGGAAGAACCCTTT
CAAGGGTTNTTTCAACCCTCTACTTGCAGAACAGCCCCTTGAGGGCTTATGACTATGGACTCTGATGGG
GGACGAGTTCANCCAATGAAAAACATAACTGGANACTTTTCCTTTCCCTTAATAATGAAATAAATACC
CNTGGGGTTTTATTTTTTACCCNAAAAAAAAAAAAAAAAAAANCCTCNGGGGGGGGGGGCG
AANACTGGAGCTCCCCGCGGTGGCGGCCGCTCTAGAACTAGTGGATCCCCCGGGCTGCAGGNAGNGG
NTGTGTGATTGCGTCCCGNAANACCGGNAGCGCTTGCNCATNGNTGACCAACTGNCTGAANAACANA
TTGCAGAATTCNAANAANCTTTTTCACTNTTTGNCCAGGNTGGGGANGGAACTNTTACNACNAAGGNA
TTGGGAACNGNNATNNAANNTCTTGGGCNNAATCCCACNNAANCNNANNTACCGGACNTNANTAATG
AAGNGGATGCNNATGGTAATGGCACNATTGACTTCCCGGAATTTCTGACGNTGATGGCAAGAAAAAT
GAAAGATACCGACACGTGAAGAAGAAATTGAGANGCATTCCGTGTGTTTGATATAAANATGGTNATG
NGCTATATTAGTGCAGCACANCTTTNCCATGTGATGACAAACCTTGTNGANATGTTANCNNATGACCA
GGNTNATGANNTGATCNGGGGACCAGANATNNNNGGTGANGGGNACNTCTACTATGGATACNTGCCA
CATGATGACANCNNAATGAAAACANTNTACACAATGTGGTAAATNTCTTGTANCACNATTGNACNTTT
GCCTTTTCTTTTGTTGNGTAACTCATCTTGTAANAANGTGTNCCCCTTACTGCCAAAAAAAAAATATC
CCTGTNTTAGTANATTAAGGACTTCCTTTCCTTCCATGGTGTCTTNCCCNTATTNTTACTGGCCNTTTGT
CCTGGAAACCTTTNTTTTTAAAAAAATTGGNTCCANGTAACATGGTTGCATTGGGGGCTTAACTCTGGG
ANAATATTNTAAANCCCCTTCTTGCCCCTCTTAAACTTTAAAANGGAGGGNGGNTCAAANNGAAGGGA
ACCAACNGGGGGGNANTCCCNTNTTTTTTTTTTTTTTTGAANNNNTNTTTTTTTTNAAAAACNCCCCCC
TCNTTTTTTTTTTNAAAAANGNN
AANACTGGAGCTCCCCGCGGTGGCGGCGGCTCTAGAAGTAGTGGATCCCCCGGCCTGCAGGNAGNGG
NTGTGTGATTGCGTCCCGNAANACCGGNAGCGCTTGCNCATNGNTGACCAACTGNCTGAANAACANA
TTGCAGAATTCNAANAANCTTTTTCACTNTTTGNCCAGGNTGGGGANGGAACTNTTACNACNAAGGNA
TTGGGAACNGNNATNNANNTCTTGGGCNNAATCCCACNNAANCNNANNTACCGGACNTNANTAATG
AAGNGGATGCNNATGGTAATGGCACNATTGACTTCCCGGAATTCTGACCNTGATGGCAAGAAAAAT
GAAAGATACCGACACGTGAAGAAGAAATTATGAGANGCATTCCGTGTGTTTGATAAANATGGTNATG
NGCTATATTAGTGCAGCACANCTTNCCATGTGATGACAAACCTTGTNGANATGTTANCNNATGACCA
GGNTNATGANNTGATCNGGGGACCAGANATNNNNGGTGANGGGNACNTCTACTATGGATAGNTGCCA
CATGATGACANCNNAATGAAAACANTNTACACAATGTGGTAAATNTCTTGTANCACNATTGNACNTTT
GCCTTTTCTTTTGTTGNGTAACTCATCTTGTAANAANGTGTNCCCCTTACTGCCAAAAAAAAAAATATC
CCTGTNTAGTANATAAGGACTTCCTTTCCTTCCATGGTGTCTTNCCCNTATTNTTACTGGCCNTTTGT
CCTGGAAACCTTTNTTTTTAAAAAAATTGGNTCCANGTAACATGGTTGCATTGGGGGCTTAACTCTGGG
ANAATATTNTAAANCCCCTTCTTGCCCCTCTTAAACTTTAAAANGGAGGGNGGNTCAAANNGAAGGGA
ACCAACNGGGGGGNANTCCCNTNTTTTTTTTTTTTTTTGAANNNNTNTTTTTTTTNAAAAACNCCCCCC
TCNTTTTTTTTTNAAAAANGNN
CAAAAGCTGGAGCTCCACCGCGGTGGCGGCCGCTCTAGAACTAGTGGATCCCCCGGGCTGCAGGAATT
CGGCACGAGGAAGACAAACCCACACGTCCCGGTGGTGGAAACACCCCATGAGGGCGCATCCTCTTCTC
CTGGTTAGTATTTATTTCAGCACCTGGTTAATGCGGTTTTTTATTCTCTACCTATTGCACTGTTGTGACT
TGTTGGCCATTATTTGATTTTTGTACGAAAAAAAAGCTTTGTTATAGAAATCAGCATACTATTTTTTTA
AACCTGGAGAGAAGATATTATGGTGACTGAAAATATGGTCAGGTGTCAAATATAAATGTATAAAAGC
CTTCTTACTGGCCTGNCTGNCATGGTACATTTATTTTATATTTGCTGGCAATACTGAAGGGTTCTTTTTC
GTTTGGGNAAACTCTAATTTCTATCACGGNGNCATGAATTTTTAAAATCAGTATTTCATTACAGGTGNC
TCAACATTGGGNAACTAANTTTTGCCAGGACCATTATTGATCAAGCAAATAAATTCAACAGCCATTTT
GANGGAAGAAAAAAAAAAAAAAAAACTNGNGGGGGGGCCCGGACCCCAATTNCCCCTATAGGGNGG
GGGNATNAANNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNC
TTNAAAAAACTGGACTCCCCGCGGTGGCGGCCGCTCTAGAACTAGTGGATCCCCCGGGCTGCAGGAAT
TCGGCACGAGGACGATTCNCTNTTTCGGNNNGTGGNTGNNGNCANGNTTNTGGANCAGGCCTTCTCCG
NCNTGGACGAGACACGGACCGCCTACATNCTCCAGGCGGTGGAGAGTGCCTGGGAAGGGGTGGACCG
GAAGAAAGCCACGCGTGCCAGAGACTCAGCCACGGCCGAGAACCCTAACGGGGTTGGGGCGGAGGCA
GAGGTGCTCANNCGGCCGTCCTCACTCCCCCAAGGAGGAAGGAGTGCCTGGGGCAGCCGCAGCCCCG
GNCCCAATTGTGTTCTGTGTGNAGCTGAANTCACTCATNTCCCANGTGANGGACCTGCTGCCGACCTN
GGCGAGGGCTTNATCCTGGCTTGCCTGGAGCACTACAGCTACAANCCCGAGCAGGTGATCAACAACTT
CCTGGAAGGCGGNTTGGCCCCCACCCTNANCCAACTGGACCGNGGCTTGGACAGACAGGTGAAGCCG
GACCCGACNCCCTTGNTGACGTTTNGTCACAACGTTTTCAAAAATGACAAGTTTGATGTNTTCAACAN
GGACTTGGTGGAACCTGACCCGGGTTGCCCCAAGGGCAGGAAGGAAGGAAAAAAAACCCCCCGGAAC
CTTTGTTNAACNAAAAAGCCGGGAAGGGGCCAGGCCCCACNGGCAANNCTTTTTAACCANTACAANC
NNTGGTGGGTNGGAAGGAAGGTNCCCCTTTCAACCCCGGGGGAAGGGCTTTNCCCNTACCGGGGGGG
GGACCAANTTTCNNNGGGTTTAGNTNCCNACNNCCCCCTTTCCCATTGGGGNACCCCGGGGGGGGCCN
CCCAACCAATTCCCGGAACTTTGNANTTAAAAAANANTTTNTTTANNNCNGGNNGGGCCNTTTTNCCA
TTTTCCTTTAAGGGGGCCCNAAAAAACCCAAAAATTNCGCCGNGGNAAANGGCT
TTTNAAAAAACTGGACTCCCCGGGGTGGCGGCCGCTCTAGAACTAGTGGATCCCCCGGGCTGCAGGAAT
TCGGCACGAGGACGATTCNCTNTTTCGGNNNGTGGNTGNNGNCANGNTTNTGGANCAGGCCTTCTCCG
NCNTGGACGAGACACGGACCGCCTACATNCTCCAGGCGGTGGAGAGTGCCTGGGAAGGGGTGGACCG
GAAGAAAGGCACGCGTGCCAGAGACTCAGCCACGGCCGAGAACCCTAACGGGGTTGGGGCGGAGGCA
GAGGTGCTCANNCGGCCGTCCTCACTCCCCCAAGGAGGAAGGAGTGCCTGGGGCAGCCGCAGCCCCG
GNCCCAATTGTGTTCTGTGTGNAGCTGAANTCACTCATNTCCCANGTGANGGACCTGCTGCCGACCTN
GGCGAGGGCTTNATCCTGGCTTGCCTGGAGCACTACAGCTACAANCCCGAGCAGGTGATCAACAACTT
CCTGGAGGCGGNTTGGCCCCCACCCTNANCCAACTGGACCGNGGCTTGGACAGACAGGTGAAGCCG
GACCCGACNCCCTTGNTGACGTTNGTCACAACGTTTCAAAAATGACAAGTTTGATGTNTTCAACAN
GGACTTGGTGGAACCTGACCCGGGTGCCCCAAGGGCAGGAAGGAAGGAAAAAAAACCCCCCGGAAC
CTTTGTNAACNAAAAAGCCGGGAAGGGGCCAGGCCCCACNGGCAANNCTTTTTAACCANTACAANC
NNTGGTGGGTNGGAAGGAAGGTNCCCCTTTCAACGCCGGGGGAAGGGCTTTNCCCNTACCCGGGGGG
GGACCAANTTTCNNNGGGTTTAGNTNCCNACNNCCCCCTTTCCCATTGGGGNACCCCGGGGGGGGCCN
CCCAACCAATTCCCGGAACTTTGNANTTAAAAAANANTTTNTTTANNNCNGGNNGGGCCNTTTTNCCA
TTTTCCTTTAAGGGGGCCCNAAAAAACCCAAAAATTNCCCCGNGGNAAANGGCT
TNNNNCAAAGCTGGAGCTCCACCGCGGTGGCGGCCGCTCTAGAACTAGTGGATCCCCCGGGCTGCAG
GTTCAGGGAGCTGTTTCCCGTATGTATCCTTCAGGGACCTGTGCATATATTCAAAGGGGAACTGTTTTT
GCTGAAAACTGCATTTTAACTGCATTCAGTATATGTCAAAAGAAGGCGAATCTATTGAGAGCACAGTG
AATTTGAAGGATCTGGAGGAAAGAAGGAAACCTTTGAATTCTCTTCTGGAATTTAAGCTATACTTCA
TCACTTAGATGTAAACCATTAGAGCCCAGGGAAATGCCTGCTACTGGTTGAGTGCAGAACTCCTTAGC
AGAGACTGGCCCAGCTGCCTGGCACCTTGATAGCAAAAGTTGCAATTCCCTCTGTATATTTTTCCCTAA
CTTGTTCCAAGTCCTCCCCTGCAGGACTTCAGAGAAGTCAATTTTCCTGTTTCCATTGTTTCTAAGAACT
TGTTGCCTAACTCAAGGTCACAGCATTTTTCTCACTTTTGTCCTATGCTTTCTTTTAGGCATTGTAGAGT
TTTAGATTTTACATGGAAATCTAGAACTTATTTTAGATTAATTTCTAAGTGATATATGGATGTATGGAA
AGTTTTCTGGTTGNTTTTTGCTTGNGAAGTATTCAATTGGTTTTGCAACATTTGCTGAAAAAGACTATT
CTTCCTTTCCTACANTGGNCTTTGCCCTGGGTGGCNACAATTATTCCTTACCTGCCCTGGGTCTATTTTC
GGGGGATTTTCCNATTTCCTTTCCCTTTNAATTTAATTNAATAAATNCTTGGGGCTTTACCAACATTCNCC
CATGGAAAANTTTTNGNAAATCCTAANGGGGNCCTTTTAAAAAAAAACNTTNGGGAAATCNCCANGG
GGAAAAAANNNAAATTCCATTGGGGGGGGGCCNTTTTTTTAAAAAAGGNGGTTTN
AANNNCTGGAGCTCCACCGCGGTGGCGGCCGCTCTAGAACTAGTGGATCCCCCGGGCTGCAGGTGACC
GGCAAGACCTCACCCTGGAGGTGGAGCCCAGTGACACCATCGAGAACGTCAAGGCCAAGATCCAAGA
CAAAGAGGGCATCCCCCCAGACCAGCAGAGGCTGATCTTTGCCGGGAAACAGCTGGAAGATGGCCGC
ACCCTGTCTGACTACAACATCCAGAAGGAGTCCACTCTGCACCTGGTCCTNCGCCTNAGNGGNGGGAT
GCAAATCTTNGNGANGACCTTGACCGGCAAGACCATCACCCTGGAGGTGGAGCCCAGTGACACCATC
GAGAACGTCAAGGCCAAGATCCAAGACNNNGAGGGCATCCCCCCAGACCAGCAGAGGCTGATCTTTG
CCGGGAAACAGCTGGAAGATGGGCGCACCCTGTCTGACTACAACATCCAAGAAAGAGTCCACTCTGC
ACNTGGTCCTNCGCCTAAGAGGNGGGATGCAAATCTTNGNGAANACCTTGACCGGCAAGACCATACC
CTGNANGTGGAGCCCAGTGACACCATCGAGAACGTCAAGGCCAAANATCCAAGACNNGAGAGGGCAT
CCCCCCAGAGCAGCAAGAGGCTGATCTTTGCCGGGGAAACAGCTTGGAAAGATGGGCCGCACCCCTGT
TTTGACTACAACNTCCAGAAAGAGTTCNACTCTGCACCTTGGTCCTTCCGGCCTTAANNGGNGGANTG
CAAATTTTNCNGGAAAANCTTGGAGCGGGNAANAACCATTACCCTNGATGTTNGANCCCANTGGCAN
CCTTTTNNGAAACGTTAGGGCCAAGAATCCCAGNAGNNNGGAGGGNNTCCCCCCAAACCCNNGAAAAN
GNTTNATTTTTTCCNGGAAANNANTGGAANATGGGCCCNCCCTTTTTGATTTNAACATCCAAAAGAAN
CCCCTNTTNNCTTTGGTCCTTGGCNTTAANGGGAGGGGGTTNAANTTCTCCCTTTAANNTTTNNAAN
GGCCGNTGTNNNNNTCNTTTNTTNNTNNATNNANTNNNNNNTNCCCCCNCNCGNTCGGCNTGCACTCN
GNNTGCCCNNNNNNCCCCCTNGGGGNGANNCNGCNCNNNNNNNNNCCTTNNGCCCCNATAGTGGGGT
CCTATTACCAANGGATTACANTNTCNACNCGGNGGTTNNTCCCCGTCCCGTCTGNCNNANTNCNCNNN
NTNCNTNCNNNTTCNNNACCCNNCNCCCCNNAGTNCCCNNAACGCGCGGGGGTCCTNCCGACCCAGN
CNNTTTNNCTGCCNCNCCACNNGNCCACNAGNCCCGNNTCNNCTCCGTNTCTNNCCCTNCCCNNNNGCN
GNGCCCCCCNNNCNCCCCCNNCNCNATACANCCACNGGNCCNNNNNNNTNNCNCNGNNCTCTTCGNC
GCNNNCNCCGGGGACNCTNNNGCNGNCNCCNNCCCCATTCCTNNGNGCCNCTNGGCCCGNNCCNTCC
CTACCCCTNGNGCGCGNNCGNCNNNCNNCACTCNCTTNATCNTGNCNCNTCGNCTCANCNCNTTCTCC
NCNTTGCCNCCCCCGTCCGNCNNTCNNGCCNNTNNCNCCCTGNGCNCNNCNNTCGTGNNTCCCNCCCT
TNNNCCCNCTNTCNCNNNGTCCCNTNGNCNCCNCGCCNNNTCCNCCGGANCCCCNANNCCCNCNGNG
CTNCCNTCANTTCNNTCNNNCCNNCCCTAGANCNTCCANCCCCCTNTCNNTATNCTCGCCNCTCNTTCT
CCACTCTCCCTCGTCNTNCCCTNCCCCNTCNTNACCNCNNCCGCCCNCGTNTCACCCTTGCNNCCNCGT
TGTCCCGCCGCACNCCCCCGNNACGNCTNGNNCNCNNTNTCTTNCGCCGTCCCGCNCAGNCAGNTGCC
CTNNCNCCNCGAGCGGANCNNNNANCCTCCTNGNNTCNNCCGCCTGTCTCNTGTCCNNCNCCCNCGNN
CCNCNGGCNNCCCCTCNTCCNCNCNNCGNCNTNCCNTNTNNNCTCCGCGGTCCTCCCNCCTNGCCCCN
NTNNCNTCCNGCCCTCNTTNNCCNGCNTCCCATGTCTTCNCCNTNNCCCNCCCNTNATCCNCGTGCTNT
CNCNNGNGANCGCNCTCNNTNACTGATCCNNGCGTANCCTGTCNGNTTCTNNCNCCCCCCNCCNNNCG
TNCCCGCNTCAGTNGCCNTCNTNCCTCCCCCNATCNCNCCTCTCNGCTCCCTCTCTCNCCTCCCTNCGA
CCNCNTCTNNCNTACACNGCGGCTTNCNNCGNNTGTNCCNCNCNCNNCNTANCCCCCCNNNTNNCCTC
NGNNCTCNNANCTCGCNACNNTNACTCCNCNCGCNNTACCCTCGTCCNNCACCTNNCCNGCTGCNTCG
TCCNGCTCCNTGGCTCNTCGNCTNNACCTCCNGCACGTCCGCNCCNCCCGTGGNCTCTTCNCGANCCNC
GCNCNNCNCNCGAATNCGGCNCNCACGCNNCNGACGCGATGNCAGGTANGTNGCNCCNTCCGCNCGN
ANNTCCCNCNCGATCGNCNCCNCGNNCNNNNATGCGACGNCCCCTCCCCGANGTCNCGNNCGCTNCN
GACGTGNCNCNCCCCTGNACGTAGCGNCCTNACCCTNTCTTTCTCCCCCNCNAGGGTCCCGCCNCTCCC
GCCCCCGCTNCNNCATCGGTGCTCCNTCCG
CTCGACAAGNGGGNGGGAAGNGNCTGCNGTGCTCTCTTTNANCAANATCTGGAGCTCCACCGCGGTG
GCGGCCGCTCTAGCAACTAGTGNGATGCCCCCGNGACTGCAGGNTCCGGCAATANGNTCNTTTTNGA
TTNNTNTAGGANTACATNNGCATATNCTTNGGGGGTCATTTCANGGTNCNNNNNCCNTNTTNAANCGG
AGNNANTNNTANNTNNGNNCNNNNGTGTTGNTNNGNNNATNGANCNTGANGGATANNTNNTNNNANGN
NTNTTTAATGGTNCANANGNGCTNTGNTNNANNTNAGNNNCTTNNNNGGGNNNAACGNNNTGNTNNAG
NANNNTANNNNNNCNGGTNNCAGNNNGACCCGNCNAATTGTTTGNTTNCNTTCCCGNGGANTCCCAN
TTCNAGATTTGNGGNNNNANGCNTGGNNCGGNGANNTTCNNTTNANNNTANAATNNGNCNCNNNNNA
CTGNCTCTNTNGNCNNTTNGNGTANNANNNCTTNAGNGTNNTNNTATNTNNTCNANNTNTATTNNCG
NGNNCAGNNTNTNCNTNANTGANTNTNGNNCAGNAGGTCGGTTATNTNCTTNTANNATACNNTNNTT
TNTCCNGCNTCNACNNTATNNATTGNAAATNTCNTCNCNTGAANNNTTNNCANNNANCTNTNNCTTTT
NTGNCCNNGCANANAAGCGTCCCNTTTNNTTATNNNACTNNNTCNNNGTTNTNACTGNCCTGGCNCTT
NNTAGTNTNGNTNNATGCTNGNNCNNGNCCNNTCCNCTCNNNGTNTNTGCTACNNNTCACCCAACANA
GCCCCCATANCACANTAGNCTCNCNNATTCCNNCANNANNAAGTTNCNTNATCNTTNNNAATATTAAT
TNCGCTTNCCGTTTTGCTANCNNTTNATCTAATNATTTGTTTANTGTGACAGNTTCCAANGNACCTNGA
NCNNCTTTTNANNGNACTCGCCGGNTGGNTAGGATCGTNAAGTCTNTNTNNNCTGCCNGTNGTNNNCN
NCTNATGTANANCATTCNNGNNTNCAGCNGTATNTNCTCTTNTACCAGCNNANGAGTNCNNATCAANA
NNTATNNNNNCNTGCCTGNTNCCATTCNAGGGTGCTTTNTGTNAANNNCNTGCCNTNGANATGNATAT
NTTTTGAACTNGTGANANGTTGNNACNNAATNGACCNTATGTNNNTGTAGTGCTCTNGCTTNGGTNTC
TNCNNATANTACNCTNCTNCNNNNNACTTNTCTCTCNTNATNGTNCGTNGCGNTNNNCNNGCGNCCG
NAAAACTGGAGCTCCACCGCGGTGGCGGCCGCTCTAGAACTAGTGGATCCCCCGGCTTGNAGGCCGAC
GCGCTTCGGTATCTGCGGGCCCCGGTGCAGACATGGCCAAGTCCAAGAANNACCCNAGCCGCANCCA
NTCCGAAAATNGNATNAGAAACCGGATTAANAAACCCCGANCNNNACNANNTNAATTTTTTAAGGGG
GNNAANCCCNANTTNCTNNAGAACNTNCCCTTTNNCNANAANCNCNACNAGAANGGCGTNAANAANA
NNCNNGCCNACNATNCCNAGGNCNTNAATGCACNTGCTNANGCTNGCAAGGGCCTTGTGAAACCCAA
GGAGANNAAGCCCAAGATNCCACAGGTGGCAGCCGCAAGCTTAANGGACTTGGCTANATTCTCACAC
CANACTGNGGAAANGCGCTCTTGCTCTCATTCCAAAGGGCTCNANGCTCTGTCCGGCANAANCCAGGG
CAAAGGTTTNCNCCAAGNNAAACCNCNTGTGGNTGTGGNTCNACTGTCAAAGGTGCCAAGGCCCCAC
AAANATCTNGAGTANAAAACCTNTTTTGTCACTGTGAAGACAACAAAGACTTGTGTGACCCCTTGTGT
TCTTGTCTTCCGNGGGGCTTGTGTCCTNCTTGTGTTNTTTTACCACATAAATCTTGTGACAGGGAAAAA
AAAAAAAAAAAAAAAAAAAATTTNGGGGGGGGGNCGGGGNCCCAAATTNNNNTTTTNTTTNGGGGG
GGNNTTTTAANNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNCT
NAAAACTGGAGCTCCACCGCGGTGGCGGCCGCTCTAGAACTAGTGGATCCCCCGGCTTGNAGGCCGAC
GCGCTTCGGTATCTGCGGGCCCCGGTGCAGACATGGCCAAGTCCAAGAANNACCCNAGCCCCANCCA
NTCCGAAAATNGNATNAGAAACCGGATTAANAAACCCCGANCNNNACNANNTNAATTTTTTAAGGGG
GNNAANCCCNANTTNCTNNAGAACNTNCCGTTTNNCNANAANCNCNACNAGAANGGCCTNAANAANA
NNCNNGCCNACNATNCCNAGGNCNTNAATGCACNTGCTNANGCTNGCAAGGGCCTTGTGAAACCCAA
GGAGANNAAGCCCAAGATNCCACAGGTGGCAGCCGCAAGCTTAANCGACTTGGCTANATTCTCACAC
CANACTGNGGAAANGCGCTCTTGCTCTCATTCCAAAGGGCTCNANGCTCTGTCCGGCANAANCCAGGG
CAAAGGTTTNCNCCAAGNNAAACCNCNTGTGGNTGTGGNTCNACTGTCAAAGGTGCCAAGGCCCCAC
AAANATCTNGAGTANAAAACCTNTTTTGTCACTGTGAAGACAACAAAGACTTGTGTGACCCCTTGTGT
TCTTGTCTTCCGNGGGGCTTGTGTCCTNCTTGTGTTNTTTTACCACATAAATCTTGTGACAGGGAAAAA
AAAAAAAAAAAAAAAAAAAATTTNGGGGGGGGGNCGGGGNCCCAAATTNNNNTTTTNTTTNGGGGG
GGNNTTTTAANNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNCT
AAAAAGCTGGAGCTCCACCGCGGTGGCGGCCGCTCTAGAACTAGTGGATCCGCCGGGCTGCAGGAATT
CGGCACGAGGGCGGTGACGGCCGAGGCGACAGAACCGAGGGGCTGTCGTTGGTGGGCGCTCCAGGGC
TCTGAACGAGAGAAGCGTGACCGGGAAACGGAAGCGAGTCCCCATCCCAACTCCGCCAGGTTTCACG
CTAGCCCCAGGCTTACTGGTAGGCTCCTGCTGCCATGGACCTGTTGTTTGGGCGGCGCAAGACGCCAG
AGGAGCTGCTGCGGCAGAACCAGCGCGCCCTGAACCGGGCCATGCGAGAGCTGGACGGTGAGCGACA
GAAGCTCGAGACCCAGGAGAAGAAAATCATCGCGGACATCAAGAAAATGGCCAAGCAGGGCCAGAT
GGACGCGGTCCGGATCATGGCGAAAGACCTGGTGCGCACCAAGCGGTACGTNCGAAAGTTTGTGTTG
ATGCCGGCCAACATNCANGCTGNGTCCCTTCAAGATCCANACACTCAAGTCTAAACAACTCAATGGCN
CAAGCCATGAAGGGCGTCACCAAAGCCATGGGCACCATGAACAGACAGNTGAAGTTGCCCCANATCC
ANAAGATCATGATGGAATTTTGAGCGGCAGGCAAAAATCATGGGNNTTGAAGGAGGAAATGATGAAC
CAACCCCCTTCATTAACCCCNTTGGGGGACCAGGNCCATTAAAAAGGGAAAGTGNTGCTTTTTGTAAC
CCCAGGTNCTTGGACNAACTTGGGGGTTGANCCTGAAAAAATGAACTTTTAAAAACTTTCCTTTTCACC
GGANGGTTTCCTTTAACGNNGGNTNGCCCGNGGNAAAAAAAACCAAAGGGTTTCACCCTTTTGNCCTT
GNGGGANCCCNAANCCCNAACCTTGGGNGGGNAAGGTTTANNAAACNNTTTNAANGGGAATTAATTT
GCCCNTTTTTTCCCCAAGG
TTNANAAANCTGGACTCCACCGCGGTGGCGGCCGCTCTAGAACTAGTGGATCCCCCGGGCTGCAGGCA
TCAGCAGCTCAGGCCCTGCTGNGACCTACCGACCCTACGACGAGGGTCTGAGGCGGNGGGTCTTCATC
ACCAATGAGACTGGGCAGCCACTGATTGGGCAGGTGTGGCCCGGACTCACCGCCTTCCCCGACTTCAC
CAACCCCGAAACTCTTGACTGGTGGCAGGACATGGTGACCGAGTTCCACGCCCAAGTGCCCTTCGATG
GCATGTGGATCGATATGAACGAGCCATCCAATTTCGTGAGAGGCTCAGTGGATGGCTGGGCGGACAAC
AGCCTGGAGAACCCGCCCTACCTGCCAGGGGTGGTTGGCGGGGACCCTCCGGCAGCCACCATCTGTGC
CTCCAGCCACCAGTTCCTGTCCACGCATTATGACCTGCACAACCTGTATGGCCTGACTGAANCCTTACG
CTCCACAGGGCCTTGGTGAAGGTTCGAGGGATGCGTCCNTTCGTGATCTNTCGCTNAACCTTTGNTGGC
CACGGCCGATACTCCGGCCANTGAACAGGGGATGTGTGGACAACTGGAACANNTTTCTTACTCCGNGC
CAAAAATCCTGNTTTTAATTTGTTGGGGGTGCCCCTGGGAAGGGGCTGACATTTGGGGNTTCTTGGCA
ACACTTNAAAAGAACCTGNGTTGCTTTGAACCAANTTGGGGGGCCTTTTTACCCCTTTTTGGCGGAAA
CCAAATTGCCCNTGAAAAAGCCANCCCGAAGGNAACCCGTTTANAAGGTTTAANNGAANACGGGNNCAA
CNAAGCCTTGAAGGAAAGGGCNTTTAACCCTTGGGGTTNAGGGGTTAATGGGCCTTATTTTTAAACCC
CTTTTTCCCAAGGGGNCCCCCNTTTAAAAAGGGNAAAAAAAGGGGGCCCGGNCCCCTTTTTTTTGGGA
NT
TTGANAAAANCTGGAGCTCCACCGCGGTGGCGGCCGCTCTAGAACTAGTGGATCCCCCGGGCTGCAGG
CATCAGCAGCTCAGGCNCTGCTGGGACCTACCGACCCTACGACGAGGGTNTGAGGCGGGGGGTCTTCA
TCACCAATGAGACTGGGCAGCCACTGATTGGGCAGGTGTGGCCCGGACTCACCGCCTTCCCCGACTTC
ACCAACCCCGAAACTCTTGACTGGTGGCAGGACATGGTGACCGAGTTCCACGCCCAAGTGCCCTTCGA
TGGCATGTGGATCGATATGAACGAGCCATCCAATTTCGTGAGAGGCTCAGTGGATGGCTGCCCGGACA
ACAGCCTGGAGAACCCGCCCTACCTGCCAGGGGTGGTTGGCGGGACCCTCCGGGCAGCCACCATCTGT
GCCTCCAGCCACCAGTTCCTGTCCACGCATTATGACCTGCACAACCTGTATGGCCTGACTGAAGCCTTA
GCCTCCCACAGGGCCCTGGTGAAAGCTCNAGGGATGCGTCCCTTCGTGATCTCTNGCTCAACCTTTTGC
TTGGNCACGGGCGATACTTCCGGCCACTTGGACAAGGGGAATGTGTGGAGCAACTGGGGANCCAGCT
TTTCCTACTCCCGTGCCANAAAATCCTGCTTTTTAAATCTTGCTGGGGGTGCCCCNTTGGTAAGGGGGC
TGACATNNTGGGGCTTNCTGGGCAACACCCTNAAAGAACCTGTGTGTTCNTTTGGACCCCANCTNGGG
GGCCCTTCTACCCCCTTCATTGCCGGAACCCCAAATGCCCTTNAAANAGCCCAGCCGGAAGGAAACCG
TACAAGGTTTNAAANGNAAAANGGGNCCANAAANGCCATTTNNGNAAGGGCCTTTAAACNTTGGGNC
TTANGGGNCATTGGGCCTANTTTTTTACAAANTTGTTTCCCAANGGNCCCCCNTTTAAANGAGGAAAA
AAGGGGCCCNCCCCCCNNTTTTTTGTGGGTACCCCCCT
TNNNACAAAACTGGAGCTCCCCGCGGTGGCGGCCGCTCTAGAACTAGTGGATCCCCCGGGCTGCAGGC
CGAACTGACCCAGAGAAGGGCAGTGTCGTCTCGGCGGCGCTGCAGAAACCCCCACCCAAGACGGCTC
CCCCTCCCCGGGCCTGTCCCCTCTTGCCCGCGAGTCCTCTCGCCTCGTAGGCCTCTCGGATCGTATATC
GTGGGGTGAGGTGAGAGCAGGCCCGGGGAGGGTGGTTACCGCTGAGGAGCTGCAGTCGCAATCAAGA
TGATAGAGGTACTGACAACAACTGACTCTCAGAAACTGCTACACCAGCTGAATGCCCTGTTGGAACAG
GAGTTGAGATGTCAGCCAAAGGTCTGCGGCTTGAGACTAATTGAATCTGCACATGATAATGGCCTTAG
AATGACTGCAAGACTGAGGGACTTTGAAGTACAAAGATCTTCTTACTCTAACTCAGTTCTTTGGCTTTG
ACACGGAGACATTTTCTCTAGCTGTGAATTTACTGGACAGATTCCTGTCTAAAATGAAGGTACAGCCC
AAGCACCTTTGGGTGTGTTGGACTAAGCTGCTTCTATCTGGCTGTAAAATCAACAGAAGAGGAAAGGA
ATGTCCCATTGGCAACTGACTTGATCCGAATAAGCCAGTACAAGGTTCACGGTTTCAANACTTGATGA
ANAATGGAAAAANATTTGTATTTGGAAAAAGGTGTGTTGGAAAAGTCAAAAGCTTACTACTGGCTTTTA
AGTTTTTTACAGGCTNTTATTACTCACTTCCCTTTNAAAAAAAACGTGCCCCCANTGNAAAGGGAAAA
AGTNNCCTTTAATTTTTTGAAAANACTTAAAAACCCCAACNTTTAANGGGCATTGCTTACTGGGANGG
AAATAATNNTTTTTTTTNAAAACNAAAGCCCCNTTTNGGGGNGGGGCNTTNGGCNANTNANCC
GAGCCTATANNGAGNCGNATTTACAGGGGCGGCCGCTCTAGAACTAGTGGATCCCCCGGGCTGCAGGT
CCGGCAGCTCCGGAGGCTTTTTGCCCAGCTGGCTGGAGATGACATGGAGGNCANCGCCACAGAACTCA
TGAACATTCTCAACAAAGTGGTGACCCGACATCCTGATCTGAANACTGATGGCTTTGGCATTGACACA
TGTCNCAGCATGGTGGCTGNAATGGATAGGGACACGACTGGCAAACTGGGCTTCGAAGAATTCAAGT
ACTTGTAGGAACAACATCAAANAGTGGCAGGCCGTATACAAACAGTTTGATGTTGACCGNTNAGGGA
CCATNGGNATGCATGTGAACTCCCCAANGGNGCCTTTTGAGGCAGCAGGATTCCCGCCTGAATGAACA
TCTCTACAACATGATCATCNCGACGCTACTCANATGANGGGAGGGAACATGGATTTTGACAAATATCA
TCNAGCTGCCTNGGTCANACTGGNATGCCATGNCCCCNTNCCTTCNANTCTCTCGACAAAAGANTGGC
ACCTGGACAAATCCAGGTGANCTCCNTNGAGTGGCTNNAANCTNGNCATGNTACTCGNTGAAACNNG
GAGCCCCAANGNCCTGCCCCNCTCATTNCCTTTGCTGGNNGGAGTCCNCCCTTNGACTTCTTCGGNCCC
CCCCCANGGCNTNATTTCTTGNNTGCAAGTNACAATCTCTCNNAGGGGTCNCTGCCNNACCCCCTTTTT
NGNCTNTTTAANCCCNTTNGCCACCTNGTTTTTCANNTCTACCANCACNNGGNCCCAANCNTGGNCTTT
TNNCACTGCCNTGNGCCCTNNAANTNCCNCNCCCTCNACGCACAANCCCATTCTTGNTTTANCTTCCAN
NAACCCTTATATTTGCACCCNGTTGNCANNCCTCCACAANCTCGNNGTGGTTTTCTNCCCCNCCCAGGG
GCGNCTNNTNCNTAACTTCTNTGNAAGGAATAATNNCTNNNCCCNTTGTNNCTAAGGCNCNTNCTTTT
TNTCTTTCCACTGTCCNNTCTCNNTNCATCTNTGCCCANATATNTTGTGCCCTTNCN
CCCCCCNNTTCNNNNGNAGANGTNTNTNNNNGNTANNNNTTNNNNNGACTCNNNNNNNTCCCNNTCNNT
NCCTNGNGGGAGGNNNCNNNNCTNNNNNNNNCNAGGGGNAGNTNTGNATNNANANNNNNNNNTTNN
GGNCTTATATGAGTCGTATTACAAANGNATNACACTCCCNCCCNNGNGGCTCTNCNNANACACGGCCG
NNANTCNCNTCNCNNNATTTTATTTTNNCNCGNNTCCANTNNTNCCTNCCCNCTCGATGGNGGGCCNT
CCNTCGCNNTNGTCNCTATNNTTNCTTTCTGNTNNGATNNCTCNNNACCNTCNAGACNNNNANTCTNT
CNAGGNGNCTANNCNCNCNTGNTNNNACANTCACCGTNGCTNCCCCGNCTNACGANTNGAGAGNTCT
ACCNNNCNNGNNANNCNTCNNANNGATCCCNCCTGCCTNNNTNGNNCCAGTCNCACNCTANTCNNTN
CCGGCNTCCCNTTTNCTCTCNNNNNNCNCCNACNNGTGNTNCNCCGTTTNCTNNCCCGCNTNNCNAGN
TGANANCCCNTNNTNANNNTCNCTGNGNNCNACNNGNCNNCATGNNGACNATGGNNCNNCTCTTTAG
AGNCGACTCCCANNCNNNGNCNTTTGNANNTCNCNNCCCCNNGNTGATNTANNNCTNTTNNCTNNNC
NCCGNCCCCTANNTACNCNNTACGANTCGNNNANNAANNCTCNNACTCGNCGANNCGTCNGTNTANT
NNCNGNCNCCNCNANNTTNNGNNCCCNGACTTCACGTNTACGAGANNNCNNGCCTNNTTGANTCCN
CANGTCNCCNNNCCNNNCTAGGTTNNTCTNNGACCGCNGCTNANCCCNCTANGANCNCCNNGAGNCC
NTGNCTNCGTNTCGCNCTTCCNTNNNNNANNNNNTGTNGTGCNACNANTGANTCCCCNNTNCNNCCAT
CNTANTATGANNNCTNCNTTCNCCNNCTNNCCNGCTCCNCCCNTGTCNNNCGAGAANNCCGTCCCTGG
TTCNNCTNTTNCCNTCGANTCCACTNNNNNGTCNNNANTANNCCNNNNCTGTNNACNNTNCGATCCNC
CTATNTGTCCTTCNNCTACTNNCAGCNCTGCTNNTCCNCNNTCATGCTCCACCACGTNNCNNCTNNNNA
GTGTGNCTNCNCCNCCATCNCCNTNNACNNTTCCNNACCCTNNNCGCCTNNGTCTCCNCANGGATNCC
CGGTNACGNCNNCGNNNCAGCTCNTNTCNCTTCGNATTNCTCNGNNCCCCCCTANCNTNNCTNACTTC
ACTCCTCTGTCCCTNTTGCCCTNNATATNCNCCACGGNNGGANNNTNGNCNTCACGNCCGNCCGCNNT
ATTNCCTCTGTCNCNTCGGCACNNGANNNTACGCATCCNGCNACCTCCNATGATCACTNNGNTCTGTC
GNNCCCCCGCANGCGTNTCGCNACGTNTCGNTATNTCTGCGTTAGTCGCNNTCNCCCTGGNTTNCTGC
ACTCTGTAAAGNCACGCTCNCNGTTNNNGTGCTCCGTATCTTNGNNTCTCTTTNCCCCCNNNCNCNCNC
CNNANGCCANGCGNCGATTGNGTGTTGNGCTNNCGAGNTAGNCGCCNCCCNCNCTCACCNTNANNGA
NNNNCNCCCNCCCCCCCNCACG
GCCCTATANNGAGNNGTATNACAGTGGCGGCCGCTCTAGAACTAGTGGATCCCCCGGGCTGCAGGAA
TTCGGCACGAGGCCGCTCGCGAGCGCGGCGGCCTCGTCTCTAGGCCGTGCAGCTGCTGCCACCGCCGT
CTGTCTGCCTGCCCTCCCGTCGGTCCACCCGCAGCATGAGCGGCCTGCGCGTCTACAGCACGTCGGTCA
CCGGCTCCCGCGAAATCAAGTCCCANCAGANTGAGGNGACCCGNATNCTGGATGGGAAGCGCATTCA
NTACCANCTAGTGGACATTTNCCAAGANAACGCCCTNNGGGANGAAANGCNANCCTTGGCCGGCAAC
CCCAAGGNCNCCCCACCCCAAANTGNCAACGGGGACCANNATTGGGGGGACTATNAACTNTTCGTGG
AGGNTGNGGAACAAAACACACTGGAGGANTTCCTGAAACTGGNCTGAACCAANTCANCCCCCTGGAC
TTTGTCATTACATTTCCCTTCCCAGGCATTACCCCGGGCATNAAGGACCTTGTGACCAACTTTNCCTGTG
TATTCCTAACCTGAACGGGGGGNGCTCTCCCCTCAANCCAAAGANCCCTTTTCNTCCCCTAANTGTAA
GCCCCCTTCTTTTTATTTAGAAAGNAANATTTNCTCNCCCACCGGGGCTCANAAAATTGTGNTTTANAC
AAACANACCNAAATGGCTGGNNTGTTNCTCANANTCGGGNCCCTGNGGGTTGTCACCCTTGNCTGGGN
CTTGTGTGATGGGGCACCTTTTNTTGATNTCCATTTATACAAAGGNTTTNTNAAAAGGGCCCNCAATAC
CCCTTNCGGGGNGCACCCCCAGAAAAAAATNAAANGGTTGCCNTTTNCATTGNGANAAAAAAAAAAA
AAAAAAACTNNGGGGGGGGGGGGGGGCCCAAACTTNNNNTNNTTNTTTTTTNGNGGGCTNTAAATNN
NNCN
GGNGGGNGGGGNGCGNAAGGAAATNTTTTATTTNNNNTNTNNNNNNTTCNCCCNCTANTTTAGTCNT
AGNGNNGNCANNTTNTNTTTTCNTCNGGGANNCCTANTAGGAACTCCCCCCNTTTGAGNGNCNNTGGA
GNNGCATTACAGGGGNGGACGCTCTNCAANTAGTGGCTCCCCTCCGTTCTTGNTACGACTNNNCCCCN
CNAANNGTTGTTTTTNTNCTCCNTGTCTATTTANNCTNNCNTCCNNNGGGGGNTTNTNTNTTTTCTCCC
CTCANTNTTATCNTTTTNCNNNGTTTNTCGGTTTTCCCGATTNATTTTTCCNATNNATGNNTANATAAT
CNANACGGACCTNNTGTTTTNTNTTNTCNACNTCNTATTCNTNTACCAGNCNCNTTNNTTTNNNTTTTN
TTCATATATTCCTNCTAATNNNCNNANATTNNNTCTCGTTGNTGTGCCTNCTCTNNCNTCTTNCTTTNC
NCNTNTANCNNNTNTCNTNTCCNCGTGCCTGTGAGTTANCNCNNCNANCNGNGTNTTTATANCNTNTG
TGNCTNTTACTNNACGANNTGTTNNCTNNGTNNNNTTTNTTTTCNNTNTNCNTNCTNNTTCTTTCGNGA
NNTNTNNNTNNTCTCTNTNGGTTAATGAANCTTNNTCTCTTNTCTCGTTNTNNTCTCNANNTTCNNNANN
TGTCCGATGNGNNTNTATNCGTNTATCTCNNNGCNTNNCNANNNNCNATGTTCTANNAATNTTTNNNN
NGTNATGNNCNNNTNCGNNAGNNNTNGGNNCTTNTGGTTTANNTTTTCTNTNTNNTNNATCGTNNTNN
CGTGTTCNATCCTNGTACNCTNATCTTTTGATANNNTCNNNGTTNTTTCGNTTCGNNNTCTCTCATGNT
TNCCCATNANGCAGCNTCNTGTCATNTNCNCTNGTCNCNTNTTNGNTCTACNGCACNGNTTANCANNC
NTANTTCCTCCTACTCGCTCCCNTNTNTNTCNNTTTTNCNCCCTNNATTCTCNCTNNAGAGNTCTCTCN
NTCNNTNTATNTNATTANGNTNNTNGTNTTANTNNCTCTCNCNTNNNGAGACNCTCTATGTGGCNTTC
NTGTAATNCTCCCNTTNATATANTATNTNNTANGACACTNTCTCTNCTCNNTNTATCTANNTCTAACTN
TCGNTCGTGGTTNNNTCNTGANNNCCTANGTANANNCNCGTTGTNCGNGNCGCTNTAANGNTNTNNTC
TTCTAGNNCGGCTNCTCTCTNACGTACNTTTGTAANANTANTNANTNNATGGAGTATTCTCCTTNTCT
AGATCGATCCNCCGTACGTNNNTAGCTATNTTCTGTANCACNTAANGATGTCTTNNCGCTNGTGTNNG
NNNTTNGTNTNTNANGTGTGTNTNTNGCNTNTGAGTGTACATANGTATGTTCAATCTNTNTTTCGTACN
GTATGNNACTTNCGTGAANNNCTCNACGANACNTANNTACGGACAANGTNNCNNANGTANCTNTNNG
TACTTACTNTTCGNANTGNTACGGTCCCTATCTATGNATGTNTNTTGCGTCGTGTNTNGTTNCTATATN
TCGNNTNGGTAGNGGCCG
GGNGGGNGGGGNGCGNAAGGAAATNTTTTATTTNNNNTTNTNNNNNNTTCNCCCNCTANTTTAGTCNT
AGNGNNGNGANNTTNTNTTTTCNTCNGGGANNCGTANTAGGAACTCCCCCCNTTTGAGNCNCNNTGGA
GNNGCATTACAGGGGNGGACGCTCTNCAANTAGTGGCTCCCCTCCGTTTCTTGNTACGACTNNNCCCCN
CNAANNGTTGTTTTTNTNCTCCNTCTCTATTTANNCTNNCNTCCNNNGGGGGNTTNTNTNTTTTCTCCC
CTCANTNTTATCNTTTTNCNNNGTTTNTCGGTTTTCCCGATTNATTTTTTCCNATNNATGNNTANATAAT
CNANACGGACCTNNTGTTTTNTNTTNTCNACNTCNTATTCNTNTACCAGNCNCNTTNNTTTNNNTTTTN
TTCATATATTCCTNCTAATNNNCNNANATTNNNTCTCGTTGNTGTGCCTNCTCTNNCNTCTTNCTTTNC
NCNTNTANCNNNTNTCNTNTCCNCGTGCCTGTGAGTTANCNCNNCNANCNGNGTNTTTATANCNTNTG
TGNCTNTTACTNNACGANNTGTTNNCTNNGTNNNNTTTNTTTTCNNTNTNCNTNCTNNTTCTTTCGNGA
NNTNTNNNTNNTCTCTNTNGGTTAATGAANCTTNNTCTGNNTCTCGTTNTNNTCTCNANNTTCNNNANN
TGTCCGATGNGNNTNTATNCGTNTATCTCNNNGCNTNNCNANNNNCNATGTTCTANNAATNTTTNNNN
NGTNATGNNCNNNTNCGNNAGNNNTNGGNNCTTNTGGTTTANNTTTTCTNTNTNNTNNATCGTNNTNN
CGTGTTCNATCCTNGTACNCTNATCTTTTGATANNNTCNNNGTTNTTTCGNTTCGNNNTCTCTCATGNT
TNCCCATNANGCAGCNTCNTGTCATNTNCNCTNGTCNCNTNTTNGNTCTACNGCACNGNTTANCANNC
NTANTTCCTCCTACTCGCTCCCNTNTNTNTCNNTTTTNCNCCCTNNATTCTCNCTNNAGAGNTCTCTCN
NTCNNTNTATNTNATTANGNTNNTNGTNTTANTNNCTCTCNCNTNNNGAGACNCTCTATGTGGCNTTC
NTGTAATNCTCCCNTTNATATANTATNTNNTANGACACTNTCTCTNCTCNNTNTATCTANNTCTAACTN
TCGNTCGTCGTTNNNTCNTGANNNCCTANGTANANNCNCGTTGTNCGNGNCGCTNTAANGNTNTNNTC
TTCTAGNNCGGCTNNCTCTCTNACGTACNTTTGTAANANTANTNANTNNATGGAGTATTCTCCTTNTCT
AGATCGATCCNCCGTACGTNNNTAGCTATNTTCTGTANCACNTAANGATGTCTTNNCGCTNGTGTNNG
NNNTTNGTNTNTNANGTGTGTNTNTNGCNTNTGAGTGTACATANGTATGTTCAATCTNTNTTTCGTACN
GTATGNNACTTNCGTGAANNNCTCNACGANACNTANNTACGGACAANGTNNCNNANGTANCTNTNNG
TACTTACTNTTCGNANTGNTACGGTCCCTATCTATGNATGTNTNTTGCGTCGTGTNTNGTTNCTATATN
TCGNNTNGGTAGNGGCCG
CANAANCTGGAGCTCCACCGCGGTGGCGGCCGCTCTAGAACTAGTGGATCCCCCGGGCTGCAGGAATT
CGGGACGAGGAGCGCTCGGAGTTTGCCACTTATCGGTCATCCTTGGTGTCCGCAGCACTCTCTCCCTCT
CTCTAGGGCGGCGACCTCCGGCGGCCGGAAAGTCACCATGTCCATCCTGAAGGTCCACGCCAGAGAG
ATCTTTGACTCTCGTGGGAATCCCACCGTTGAGGTTGATCTCTTCACCGCGAAAGGTCTCTTCAGAGCT
GCTGTGCCCAGTGGCGCTTCAACTGGAATCTATGAGGCCCTGGAGCTTCCGGGACAATGATAAGACGC
GCTACATGGGGAAAGGTGTCTCAAAGGCTGTTGAGCACATCAATAAAACTATTGCGCCTGNCCTGGNT
AGCAAGAAGCTGAACGTCGTGGAGCAAGANAAGATCGACAAGCTGATGATAGAGATGGATGGCACA
NAAAATAAGTNCAAGTTTGGTGCGAACGCCATNCTGGGCGTGTCCCTGGCTGTCTGCAAGGCTGGTGC
TTGTGGANAAAGGGGTGNCCCTNTANCGGCACATNGNCGACTTGGCTGGCAATGCTGAAGTCATNCTG
CCAGGTNCGGCTTTTAATGGCATCAACGGNGGCTTTNATGCTGGCNACCAAGCTGGGCCTTGCCGGAN
NTTATTAATCCTTTCTGGTGGGGGCCCAAAACNTTCCGGGAGGGCCATTCCCCATTGGAACCAAAAGG
TTTANCCCCAANCTTGAAAAAAGNTCTTCAANGGANAAATTTTGGGAAAGGATGNCGCCCCACCCTNG
GAAAAAAAAGGGCNGGTTTTTNCCCCCAACATTCCTGGGNAAAAAAAAAAAAANCCCTTGGAACTTT
NTTNAAAAAAGGCCCTTTGGCAAAGGGTTGGGTTTCAAAGGNAAAANGGGCTTNATTNGGGAATGGG
CCNNAAACTTGCCCTTTAA
TTNANCACNATCTGGAGCTCCACCGCGGTGGCGGCCGCTCTAGAACTAGTGGATCCCCCGGGCTGCAG
GNCACGGANGCCCTCANCATGGAGGGAGCNCGNCCGCTGNCGGCGGCTCGCGGCAGCNTCTGAAGAG
GCCAAGGCGGACGCCCCCTCTGGGCACAGTGGCTGGGGGTGCGCACGGNCTCANGGGCTGGCGCACA
CCTTTCTCTGTGAGGGCGAGGGCTAAATATAACCCGGAGAGTGAAGCCAGTGATAAGCTGGGGCGTGT
CTGGNCCTGANTTCTGTTGAGACCTGGCGGNTAGCAGGGCCCAAGCTAGGNCCGGCTGGGAGACGTTA
GTGTCTCCTTGTGCTAAAGGCCTGGCTGACCTCACCCACAAGATCCCAGCCTCCCCACTATTTATAGCC
CCAGAGGCCCTGGGAGGGTAATGTGGGGGCAAGAGGAAAAAGTGGAANGAAGGTGTGCATTTGAACC
TTTTGAGTCTTGGTTATGCTGCTAAAAAACCACATGACCATGGCAAGTTGCCTTAGGCACTTCACCTTA
ACCTTCCTTACTTGGGCAAGTGGGGGGNGATGGNCCACATATAAATGCTTGGGGAGGNCCATTTGGAN
AATCACACAAGAATGCAAAGTANCAGGTGCCCGNTTTTGTTTGATTGAACCACAGGCTTTCCAAATCC
AAGCCACTTGTNAGGCCTTTGGGCCCTGAAAATTTCTTGAAACNAACAACTTAGGTTTTTCAAGGGAA
GGCTTGGNCCTGGGCCTNAAAAANNTTTTGNTTTGCATTTTTTTTTGAANCAAAAGGGGGTNNAAACG
NTAAAGGGCCTNCCGTTTTNAAAGGNNAAAAANAATTCTTNGGGNCCCAAATTTTTTAAAAAAAAAG
GGGTTTGNCCCCCCCCNAAAATGNATTANGGGGCCTGGGGGGGGGGTTTTTAAAATNCAAANGGGGG
GGNCNCAGNTTTTTTTTTTTTTTN
TNNANAAAAGCTGGAGCTCCACCGGGGTGGCGGCCGCTCTAGAACTAGTGGATCCCCCGGGCTGCAG
GCTCGAGTTTTTTTTTTTTTTTTTTTTTTTTTTTTTTTTTTTTTTTTTTTTTTTTTTTTTTTTTTTTTTTTTTTT
TTTTTTTTTTTTTTTTTTTTTTTTTTTTTTNNGGNNNNNCCNAAANNNNNNNAAAAAANTTTTTTTTTTTN
NNAAAAAAAAAAAAAAAAAAANTTTTTTTNNNNNGGGGGGGGNNNNNAAAAAAAAAATNNNNNNNT
TTTTTNNGGGGGGCCCCCCCCCCNNTTNTTTTTTAAAGGNTTTNNNNNANNGGGGGGGNTNNNAAAAA
AAAAAANGGGGGGGNAAANNGGTTTTTTNAAAAAAAANTTTTTTANNCCCCCNGGGGGNNAAAAAAA
AAAAAGNGGGNTNNAAAAAANGNGGGGGGGGGGGNCCCCCCCCCCCCNCNNNTTNTGGGGGGGGGG
GANNNNNNANNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNCCGNNNNNNCGNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNCNCNCCCCCCCCCCCCCT
TTNNNNAAANCTGGAGCTCCCCGCGGTGGCGGCCGCTCTAGAACTAGTGGATCCCCCGGGCTGCAGGT
TCCTGTAACTCTTAATAAACTTACACATGCAAGCATCTACACCCCAGTGAGAATGCCCTCTAGGTTAT
TAAAACTAAGAGGAGCTGGCATCAAGCACACACCCTGTAGCTCACGACGCCTTGCTTAACCACACCCC
CACGGGAAACAGCAGTGACAAAAATTAAGCCATAAACGAAAGTTTGACTAAGTTATATTAATTAGGG
TTGGTAAATCTCGTGCCAGCCACCGCGGTCATACGATTAACCCAAGCTAACAGGAGTACGGCGTAAAA
CGTGTTAAAGCACCATACCAAATAGGGTTAAATTCTAACTAAGCTGTAAAAAGCCATGATTAAAATAA
AAATAAATGACGAAAGTGACCCTACAATAGCCGACGCACTATAGCTAAGACCCAAACTGGGATTAGA
TACCCCACTATGCTTAGCCCTAAACACAGATAATTACATAAACAAAATTATTCGCCAGAGTACTACTA
GCAACAGCTTAAAACTCAAAGGACTTGGCGGTGCTTTATATCCTTCTAGAGGAGCCTGTTCTATAATCG
ATAAACCCCGATAAACCTCACCAATTCTTGCTAATACAGTCTATATACCGGCATCTTCAGCAAACCCTA
AAAAAAAAAAAAAAAAAATTNGGGGGGGGGCCCGGNNCCCAATTTGNCCNTATAGGGGGGGNNNNT
NAANNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNN
GCNTATNTGGAGCTNCACCNCNNGGGCGGCCGCTCTAGAACTAGTGGATGCCCCGGGCTGCAGGNCN
NCGNCCCGNCNGGGGNTGCCGGCATNGCAAGAACNANCCGNACNCAGNGNGNNNCTTCNGTCNATGT
GNACNTGATGCTANAATTNATATNNTNGNCTNNGNGCGNANNAAGNCNAAANCGGNTGAANCCCNAC
TNTNTNGNNNNNNNNCNGNNAANNANNNTNANCAGNTTTGATCTNAAACCTTGTNGGCTGCGANTAT
TTGTACCNCCNANTANNTGNTGANGANCTGAGACNGAAAGGGNTNNCNCATACTNNTGNNGCTNCAN
CCGTTANATGNNATGCGNANGANCNNGATGTNGTGTTGTGCTGNATNTNATACACTNCNCACNGGTAN
NCGCNNTGNNNTTNGAANGNCNNNNGNNNNANGGNCNNGGNNNCNCNTTGNNCATGTCNTAAAGGG
CNTCNGAACCAAANNNGAGNNCNNNGNACNTGNNANTGAATGCCTNCGACGGNCCNANNNNAANCT
NCNNNGGCGACAAAAAATCNNNNTGNATCAAAANNGNNGNTTTANCAANNTNAACGCGGATGAANN
TCNTNCATGNCGGNATAAANGNGANNTATACCANANTGANGCNGGGNCTAATNAATTCCTAANNAT
GGAANCGTGTACCAATGGCNGCCCTCCTAATTAGCCTCAATNCTTTCCCTACTTNTGANANNCTCCAN
TAAAATGCTAGGNTNTTGTAAAAAAAAAAAANAAAAANTTGGGNGGGGGGGGNANATTNCCAA
AAANAACCTTTAAAAANGGNNNNCNNAAAGCAAATTTTAAANGCNCTTGNANAATTTNGNAANGG
GAAANNTNTGGAAAAAANNAAAAAGGCCCNNATTTNNNCNNAAAAAGGNNNTTTNNNNANCNNATN
AAAAAAATTCNGGGNANCCCAAAACCNGNNNCTTTTNNAANAANNNNNAAAAANCNCNN
NCNACTGGAGCTCCACCGCGGGGGGCGGCCGCTCTAGAACTAGTGGATCCCCCGGGCTGCAGGAAAT
TCGGCACGAGGGCTGGATGCAGAATAGGATAATCTGGAGAATGGAGGAAGAATGCAAAACGATATAG
TATCCCTTATGGATGGTACATGTGCAACAGGGAACTATTTCATATACCCTTGGCAGTCTCTGATAATCA
GGGAGGAAGAAAAACCTGGAATTTGGATTAAGGCTGATCTTTCTGGTTTTTGCGCACTGTGGCTCTCCC
AGGCATATATAGTGAAGGTGAATGTCTTCTCCCTCAGAAAAACTCAGGTTCCTTGCTGTCCTGATAAG
GCATAGCTTCCCTGCCGTAACTTAAAACTCAGTGAGGACTTAAGAGGGAAAGAATGAGGCAAATATAT
AGGACTGCAGGATAACAACTGCATTGTTGTGTACTGTGGAATGGGGAGGGCACTCTTGGAGGAGGACT
GCTGAAGGTGGTCAGTCTACCTGAGAATGGAAGACATACTTGGTGGGGAGCANGGTATGTGCTTTTAT
ATGAAAAGAGCCGATGTTAAAACTCATTTGGTTAGGTAAACGTTGCACATACCTTCACATAANGGATA
GTATATTTTGGGTTGCAGTCAAACTTCATTGTGCTCAAACTGGTGAAAATGCAGTTAGGCTTTTTGTAA
ATACCAAAAAAAATCCGGTCTCACTCTTAATTCANGNGGCTACTGCTACTTCANAATGATTTTAAAAG
TCCNCCCCCCCTCCCCCATGAANNTTCTTCCTATTTTTTATGCCTGGAACCTTGCCCCCAANNTTTTGTT
TNNGGAATTTTACTTNTTTAAATTTTGAAAATNANCNTTTNTAAANNTCNAGCCCCTTGNNTTTTNANA
NTNANNNTTTTNTTNTNTNCNTTANNNAAAAAAAAAANNNNNNNNNNNNNNNAAAAACNGGNNNN
TNNTTTCCTTNNNANGGGNNTTTNNNNNCCANCT
TNNNATAGNGAATNGNTANGANNCNACCNNCTNNNCCTTTGAGCCCNATAGTGAGTCGTATTACAG
GGGCGGCCGCTCTANAACTAGTGNATACCCCCGGGCTGCATGCTGCCCNCNCGTTGCNCTCTTTCNG
CTCGCCCCCTGTCCCNGCNCCCCCCGGGNTNTTCCNCANCNCATCCCGTNTCTATCCCNNANTCCANA
GANANANTGANNTTCNCTTGNATNAACGAGGAANCATNTNGTCCTAAAAATCTTTCCNTTNTNNTNGN
CCNNANNNGAATNCTNANTNAANTANNNTAGNACTCGGGCTNCCCGGCTTATNTATNNTAAANANGC
CNCCTNNCCGCTNCNNCATATGTTNNTCCTTCACCACNNNNANTNTCTCCTTGNTNCGTTCNNNGTNN
CGCCCCANNTCNGCNCTNCNNACTAANCNCCCNAANNCCNTNNNNCNNCATTCCNNNTAACNANGCC
CTANGAACTNTNTNNTNCTTTCTATCCNNCACACCACCCNTTNATTTCTNGCTTCACTNNNNGCCNCC
TNTTCNAGNTTCATAATTNTTCCTNNTGTANNCTCCCTGCNAANCCAAAAANTNTTNNTTNTTCTTCN
CCNTCCNNANANCTNNTNNATANCATCTGCCANNGNCNCCTNCGCNAANTCTGCNCTCTCNNTNTNA
TNTATGCCNGNATACCTTGTNCTGCTCATAATNATNATANNTTNANATNNNCCCTNTNATTTTCTCNAA
TCCNGTCTCCTANGCGNNNCTTAANACANCACNATTATTTTCNCCCNCCNCNNTCCTCNCTCTCCCTC
CCTTTCTNCNNNCNNGCCCNCCCCACNCTTTCCCCTTTCNTNCNTCCACGCCCGTCNNTCCTANNTA
CTNNGCNCNCTNNTTNANCTCCNNCCCCNCCTCNNTCTTCCTTNCNCNNTNNTCTTNNTNTCACNCNAT
ATAANCGACNNCATAANTCTTACAGNTCCCTNAAGANTNTCACCNTCATATCTCTNTCNCNNTCCTCN
NCNANTCCNTATCANNCGCGCTNCTTTATTTGAGTTAATTTGTNTTGTTNTCNTANCCGTTNTNGNNCN
TNCTTTCCNGACNCGNNNNNCCNNCTCNCCNTNTANTNCGCNCTNCTCTTNATCTANTTCTNTCNTCAC
CTNANNNTCNNACCNCACTCCTTCAGNTCNACCGTNNCGANNNNTCGCNCTCATTCANAGAGNGCNCC
G
TNAGNCCTATAGTGNNGTCGTANTTANAGGGGCGGCCCGCTCTAGAACTAGTGGATCCCCCGGGCTGC
AGGAAATTCGGCACGAGGGCGTGACGTGCNNTNGCCGCCACCTGGTNNNCATGGGNCANCACTTCCT
GTTAAGANAANCCACNANGTGGAACGGNGACGANNTANACTNNNTNATCNGCTNTNTGNCAGGCTGT
CANGAATCANATAGGAGTNCCGTNCCCAGNACATCNNCNTTCNNNNNNTGCTGNTGNTNAACNANG
ATNNGTTNAANAGNGNCNNNAACNTNTAGCTGGGGGCACAGATTANNATCTANTNTGNNTTTNANCN
GCTCNCGGACNCTTANNGNAAGGNCGNGNNCANGACNCTTNTNCTCCGANNNAGCAGACCCGCACC
NTTTANACAGAGGGTCANNAACNNATANNTTTNTGTTGTGTTGANACNNGAACACCNACCTCTTTNNN
CNGNNGCCGCAGANCCNCAANACTNTTGTTCGTNTGNNCNNGTANCNCCCCCCTCCTTTNCANNNAGG
GCATNNNAGCANAANACTANTNTTGTGNNNNTNAAACNTGGGTGNCCCNTCNCTCTTTNNACAATTAN
GTGNTNACAACTGNCTTCNNTTCTAAAANTNNNNCNTAANCGACNAAGANTNNGTNNNTCNATATNN
NTTGTTCTCNNTTNCTNTNTNNCANANNNGATNTTNCTTTCNTCNNGTCTNTNCTCNCNNTAATTTANN
TAAAATTTNCTCNTGNTATACNAATTTTNCTTNNTNNNTGGCNNGCNCTNNAANCTCCCTNGAACTTNT
NTTNANTCNTTTNTNNTNACNTCNNTTNAAACACTCTTNNNTNNTNTTCTTNGGTNTTANNTTNNCTNT
NTTTNANCTTGTNNCTAGAGTTTTCCCTCTATTCTTTTTTTATNTANANNTCTNNGTCTATTTGNGNTNC
GGNGTCTAGNNNNTNNATTNTCTGTNCTCCANTANACTNANANGACTNTTNCTGTTTCTNANCTATTTT
TCTTNTNCNATCNTCATCCCCG
TNAGNCCTATAGTGNNGTCGTANTTANAGGGGCGGCCCGCTCTAGAACTAGTGGATCCCCCGGGCTGC
AGGAAATTCGGCACGAGGGCGTGACGTGCNNTNGCCGCCACCTGGTNNNCATGGGNCANCAGTTCCT
GTTAAGANAANCCACNANGTGGAACGGNGACGANNTANACTNNNTNATCNGCTNTNTGNCAGGCTGT
CANGAANTCANATAGGAGTNCCGTNCCCAGNACATCNNCNTTCNNNNNNTGCTGNTGNTNAACNANG
ATNNGTTNAANAGNGNCNNNAACNTNTAGCTGGGGGCACAGATTANNATCTANTNTGNNTTTNANCN
GCTCNCGGACNCTTANNGNAAGGNCGNGNNCANGACNCTTNTNCTCCNGANNNAGCAGACCCGCACC
NTTTANACAGAGGGTCANNAACNNATANNTTTNTGTTGTGTTGANACNNGAACACCNACCTCTTTNNN
CNGNNGCCGCAGANCCNCAANACTNTTGTTCGTNTGNNCNNGTANCNCCCCCCTCCTTTNCANNNAGG
GCATNNNAGCANAANACTANTNTTGTGNNNNTNAAACNTGGGTGNCCCNTCNCTCTTTNNACAATTAN
GTGNTNACAACTGNCTTCNNTTCTAAAANTNNNNCNTAANCGACNAAGANTNNGTNNNTCNATATNN
NTTGTTCTCNNTTNCTCTCTNNCANANNNGATNTTNCTTTCNTCNNGTCTNTNCTCNCNNTAATTTANN
TAAAATTTNCTCNTGNTTTATACNAATTTTNCTTNNTNNNTGGCNNGCTNNAANCTCCCTNGAACTTNT
NTTNANTCNTTTNTNNTNACNTCNNTTNAAACACTCTTNNNTNNTNTTCTTNGGTNTTANNTTNNCTNT
NTTTNANCTTGTNNCTAGAGTTTTCGCTCTATTCTTTTTTTATNTANANNTCTNNGTCTATTTGNGNTNC
GGNGTCTAGNNNNTNNATTNTCTGTNCTCCANTANACTNANANGACTNTTNCTGTTTCTNANCTATTTT
TCTTNTNCNATCNTCATCCCCG
TNNNNNAAANGCTGGAGCTCCACCGCGGGTGGCGGCCGCTCTAGAACTAGTGGATCCCCCGGGCTGC
AGGAATTCGGCACGAGGGTGATCTTACGTCCTTTTTAAAAGCCCTGCTGCTGCTGCTGCTGCTAGTCGT
GTCTGACTCTGTGCGACCCCATAGATGGCAGCCCACCAGGCTCCCCCATCCCTGGGATTCTCCAGGCA
AGAACACTGGAGTGGGTTGCCATTTCCTTCTCCAATGCGTGAAAAGTGAAATTGCTCAGTCGTGTCCG
ACTCTTCACGACCCTATGGACTGCAGCCCACCAGGCTCCTCTGTCCATGGGATTTTCCAGGCAAGAGTA
CTGGAGTGGGTTTAAAAGCCCTAGGACTCTAGGAAAACCTCTCTCTTCATCTAGTGTGGAAAGCTTATT
AAGAAAGGAGTGGAATGAGCCTTACCAGATGGTTCCCAGGTGGTGCTAATGGTAAAGAGCCCACCTA
CCAATGCAGGAGACATGAGAGACATGGATTCGATCTCTGGGTTGGGAAGATCCCCTGGAGAANGAAA
TGGCAGTCTACACCGGTATTCTGCCTGGAGAATCCCATGGACAGAGGAGCCTGGNGGCTGTANTCCA
TANGGNCACAAAAAAGTNTGAAGTGACCTAACATACATGGGAGNTAACAGGACATTTTAAGAAAAAA
GGAAAAGCTTAACCNAAGGCNGGCTGTNCAAATCGTGAGAAACTTNCCCNAAAGTNTTGTCTGAGNT
ATAAATGCCTGAAAANCNTCNCCAACCCCTTTTCTTTTGGAAAAAAATGGCGGNGTATTTTTGCCNNC
CTTTGAGCAACTTGAAAATTTTTTTTTTTNAAACGAAGCCNCTTAAAATNGGNAANNNTTTNTTGGGGG
NAANCCTNAAAACCTCTTTTTTAAAAGGCCCCCTTGNTTTTTAAGGGNTNGGGGGCTGGGGAANAAAN
TGGGCCNAAAAACCTGGGGGGTTGA
TNNAACAAANCTGGAGCTCCACCGCGGTGGCGGCCGCTCTAGAACTAGTGGATCCCCCGGGCTGCAG
GTTTCAGTCCTTTGGATCTACGTTTCCAATTAAGTATTAGTCAAGTATTAACAGCACAAGTAATGGGTT
AACTTTAGACTAGGAATTGGTGTTGGGGTGGGGGGTGCAAGAATCTTTATTTTATTTTATTTTTTTGGA
TAAAAGTTTTATCTATTATATATTAAACGTTCTTGCTGCTGTGCTGCAAAGCCATAGCAGATTTGAGGC
GCTTTTGAGGGCCGAATTATTCTCCAAGTTGAGAGATGTCCTCGGGTTGAATTGAAAGCCCTACCCAA
AACTGCAGTGGGAAGGGGGAGAGCCTTTGCCTCCACTGCCCCACCCTCACCCTCCTCGTAGACCCTCT
GCCTTTTGAAAGCAGATCATTTTCACTGAGATGTTGGACATTGCAGATGTCTGTTCCCCAGGCCAGCAG
GGACCTCTGAAACCTTCATGGCCTGGCCTTTTTTTTTTTCAANCCNGGGGTTTTCCAAAGGGANACCCN
GGATTTTGGAAATCGAAAANTTTTCCAAGCTNTATTTCTNAAATTTNANAACCTTTATNCNAAGGTNA
AATTGGAAGGGCTTGTTTGNAAACCTTNACCCCCCTTGAAANCCCCCCCTTAATGAAAAAACCTTGGG
GGGTGGCNTNAAAAAAAAGGANGGTGGGNATTNAAAGTTTAAAAANNNCCCGGTTTTTTGAAGGCCN
AAATTNCCCCTTGGGGAATTAAANNATTTCCNGGGTTTTCCTTGGCCCCNTTTAACCCTTTGGTGGNCC
TGGGGAAGGGAATTGGGGGNAAAAAAAAATGGTTGGTTTTTTNCCCCAAACCTAACCCCCAATTTCNT
TGGAATTAAAAAATGAAATTTTNAAAGNTTTTNNTTTTTGGCAAAAAAAAGGGCCTTTTTGGCCGGNT
TTGANACAANCTGGAGCTCCACCGCGGGGGCGGCCGCTCTAGAACTAGTGGATCCCCCGGGCTGCAG
GCTCNNGNNACNANGTTTTTTTNNTTNCNGCTGGGNTGNAGCTGTNCNCGGGTNNGNCNATGGNNCCCA
CCTGGNGGCTAACCTGCTCCTCCTTGGCTATGANCCCACCAGGGACATCCCTGGCTCCTGNTGGCCGTA
GNGNGCTGGCATGGCCCACCTNGGGGCTNACCTGCTGCNNGCNGACTTGCCTTCCCGGCCTGANCCTGT
NACCTTGNTCNCTACTGGTAACCNNTCCTTNACNNGNNGCCTTTNCANGGGNCNTACCCCANGGGANT
GATTCTTCTGCNCTGNTGCTACCTTNGCTTATTTCTTGGNCGCTTNGGTTTCCTNNCTTGGGTACANACC
CTGGTNTTCTGNTGGNNTNANGAATGCTCNGTCNTGNGCCNGNAANTGAATCTTCNCTTGGGGNNAAN
TGANNANTCTNTTTNNANGGGGTNNNTNGAANNCCNGTNNTACCTNNGCNTTNNGGNAAACANANANC
NTGGATNTCTNGNCNCGCNTGNNCTTTGGGNTCANAATNCCATGCNNTNGGGNNANAACCTANNNCN
TAGNGTTTNNGNANCCCCNNCNNGGGGGGNNTNCNCCTGNNTGANANTTTTNNTATTNAANATCCTTT
NCGGGCTCTNAAGGGNTTANNNNGGNGCCCCNCAACNTTTCACNATNTCGNGNNNGCCCNCNANNAA
AANAAAANCNNCCTNNTTTCTTTNCCCNGTCGCTCNCAANNACANCCCNNNNNNGNCNTCANNAANGN
NGGNGCTTCNNTNTCTNCCTANANTNNNNNNNNNTCNNCCNANNNTCAATGNCNGGNAGTATANANN
NNTNATTTTNTNGNGCANCTCCGTNTNCCGTGTGNNNCATCTCNTCTNNGNCNNNATATNCNNGCNNG
NTNCTTNCCNACNTNNACTTNNTNNCCNATCNTTNNGTCNCNTANNTNTNANCNCANGCTGCNNCCTC
TCCAACTNTNCATTCTCGNCNCNGCNNTTCCNNTTTNNTCANTANCANCNNCNCATCNTACTNTCGNTN
ANATGCTNTGTNNTNCTCTGCTTNNCNCNTTTCNCTCNNTNCCA
TTNANAAAACTGGAGCTCCCCGCGGTGGCGGCCGCTCTAGAACTAGTGGATCCCCCGGGCTGCAGGAA
TTCGGCACGAGGGTCCGAGTCGTCTGATGTCCTGTCCAAGTGAAAGGCTCCTGCGGTCCCTCCCAGCCT
CTCCGGCTCATTTGGCTCCTGCAGATGGAGCTGTGCAGGGGAGCATCTGCACAGGAGACCTGAGGTTTC
GCCCCTGTCCTCCGGCCACACCTGGGGGGAGTCGACTGCCTGGGGTTGCCCCTTTCGCCCATGACCCGA
CCTAAAAGCCAATGTAAGCGTCTTTTTCAGAATAAATCCAATTCGAGTAAAAAAAAAAAAAAAAANN
TGGGGGGGGGNCCNGGNCCCNATTNCCCCTTNTNGGGNNNGNNNNCCCNCCCCCNNNNNNCCNNNN
NCCCCCCNNNNNNNNNNNNNNNNNNNNNNNNNNNNNNNNNNNCCCCCCCCCNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNN
ANNACTGGAGCTCCACCGCGGTGGCGGCCGCTCTAGAACTAGTGGATCCCCCGGGCTGCAGGAATTCG
GCACGAGGTCCTTTATTCCTACTCAGACTGTTGCACATACTAGCCTTGGGAGTTCCTAGAATAGAAATC
CTTAGGTTTTAATTCCAGTCCTGTTTCCACCTACAGAGTCCCAACGTGGTTATTGCAGGCAACGTTCCC
TCTCTCCGTGGTCTAGAAGCCCCTCCCCCAAGGTAGAAAGAAGGGAAGAAGCTAACTCCAGTGTTTCC
GTTGCACTGATCCCCAATTCAGTCGAGGAGGGGGCTTGGTAACCCCTCTCCCTCAATATCCTGGCACCT
TGGGCTTTGTGAACGCCTCCTAGCCAAATCACTAGAGTACAGTGACCCCAGCCTCCTGCCTGTCGCGAG
TGAGCCCTCCCCACCCTGACCGTGCTAACTGTGTGTACATATATATTCTACATATATGTATATTAAACC
CGCACTGNCATGTGTACCCTTTTTCTGTGGNGTCTAGCATTAACTTATTGTCAAGGCCCGGGCGGGGGT
GGAAGGAGAATGCCACAGTGAAGGGAGTGGCANAGTCAAATTGCTATATAGTCGAAAAAGAAAACTT
TTTTAAACTTTAATTTCACATGCAATGTCAAGAGACTATTTAGAGAGTTTAAGTGAGGNGGGGGAGCT
TTTGGGATGTTGGGGAACAAAACTTCANTGGGAAGGAAAGGGCTGGTTGTTGGAGAAGGGAACAANC
CCACTGGNTGGAGAAGCACCCTTTTACAANCCAAGCCCANACCCANACCGGTNCCCTTTNGCCACTTG
TGGGGGACNCCTNCCTTTAAAACANACACCTTCNGNGGTNCTNCCACAAGTTTTNCCTTAAANGAACC
TTCCCNAGATTTCCCCNACCCNTNTCTNGGGTTTCTCCCNCNAGNANGAGAANANAANCNTTTGANAA
NTTGNGGANGTGGAAAANNAACNTGAATTTNTCCNTTCCAACNAGGCT
GNCCNATNNGGGANGNNGCATNACAGGNGGCNGGCCGCTCTAGTAACTAGTGGATGCCCCGGGCTGC
AGGNAATCNCGGCCAGGAGGGCTGNTATGCANCAATANGCATTAATCATGTTAATAANNGGNGGGGG
AGGAACTACTTTCATGATAGNGNNANNNNNNNNCTCGNGATNNGANACNANAGGGCCNCTNTTGCGA
NNANTTGCNNTNTACNAGCNCNGGNCTGTNACTCNCNTATAANCACANNNNTGNTNAGTGNNTNNCC
NAANCTCGNTNATCNGAANNNTNTGNNAACNTGCANTGTNCAAGAGACCNNATNCTATGNNNGNNNN
TGGGTNGACCTTNCANTCACGCGTNNNATNNANTNCGANTGNNNGTCGATCTNCTCNNNNCATGGNNT
CTNATATNTCATGNGNNNGANAGTCGNATCATNANNGANTATANNTANNGGANTTNCTNCTNNGATAT
TCTTNCCNCNGNTCNTNGACANGACGCACACGNACNNCACACGCAGTNNGTNNGNANGNAAGATNCN
TNNTATNGCCACNANAGNNNGNAAGANTANACGAGTTNTNNACATCNTNAGNGNGCGATACNCNAGT
TCNNNACGNNTTANANNNAGTAGCGNNNGNGNTCNCNAGTTTTAATGTCNCTNGNCANNGNCNNACN
CANNNNNNTCATNATTTNNTCNNNNNANATGNCANCNNATNAGTNNGTNGCNNCNNGCNNNNNNCGT
GANNCCCANATGNTNCNTNCATGATCTCANTTANANNGGAGTNNTGACAANANTNTNTTGANANTNN
GTTNACAGTCNAGNAGANNCCATCAGNNNGNNACANATNNNNGNACNCTNNTGTTTTTTNNNTCGAC
AGTANTNTANGTTNTNTNNCNATAGAGNNNAGTAGTCTGNTNNCAGANACTCTAGNAGACNCNTNN
GNNCCTTNNTAGTTNATANTNNATNNTNCNCGTNNGACTCNNNNANACTTCTNNTCNACTTTTNNC
TGCNTNTGTCNCAAGANACCGATTGTGATNANCCTANNNTNACGTNTTCGGAGGAGANNNNCGATTCC
NCNACTNTNCNNCCANGGTCTNCCTNGTNGCCTCNGACNAACNNTATNCNCTCCTGTTAGTAGCATNN
ACGCNGAGNNGNTANTTNNATNTTGNAGCTTCATGTTNATGGTAGTNTGATGTANGTAGTAGGAATGA
AGAANNTGGAANTAGNGANAAATAAANAGNANAAGTCGNAAGCANAAANTAAAGGANGNNNGACG
NACGCNATCANTAACCTCNNTNNNNATNTCGTTCCTCCGTCT
TACTAANGGGAGGGACNCANNNAACNCNNNANNTTGGNGCTTGAGGCCCTCATAGTGAGTCCGCATT
ACAGTGGTNNCCGCCCCTACTNTNNAGNGTGATCNAACCANANTACCATTACNCATTATTTCCCNNNT
AATNTTTATTTATCTNCACNANNACAANCTCTGATANCNTNTTCGNTCNNNGGGNTTATCNTNTGCNN
CTNGGTTNAANNCATNNACCCTANATCTGNCNCNANCCCTTATTCTTCAANNTANGANTNCGNNCNC
ATCNTNATTATNNATAGANNNNTNTANTCTNNNNACNGNAATCAAAGCNNCNNTNTNTTTTCTNTNNT
NTTATNTCNCTCTTGNNGANNCCCCAGGANNCGNTNCANTTNTAATTATATGTCNCTNTNCTNCNTTNN
NTTTNNTANTTNTNTTTTNTTCTTACGCGTNAACNANTGTNCNATATANNNTTTGTANCGATNAGGCAC
NNNNNNNATTAANTTTNGTGTCTATACTNTNNNCCGNNACNNCGNAANNCCATTTTCTATCNTNGNTNT
ANNCAACNTCNNCAATNTNNTNNNTTNNNNANNTNNNCNNCGANACANNNNGNNCACNTTNANNCTA
TTNGNCCNNCTNNCCCAGACNNACNNGGCTNNTCCTCNNNNTAATANNTNANCTNCATGGNNNCTATT
TNGANTATNCNAAATANTNTTNNTATATNATNCAAANNNATCANGTTTNNCTATAAGTTATNNANNTC
AAGNGGANNNANTAANACGCTCCANGCTANGTTNTCTCNNGANNCTNTGAGATATAAAANNNNTNTN
NAGNNCNNNGATANNGCAATNTTNATNGTTTGNCCTTNTATCCTATNGNNTATNNTNTANNNNNTNNN
CTNGNNTNNNTNCGTCNTCAATNTTNANTNTNATTTNTNNTNACATAATTTTCACCTACTCNNCATNN
NATNNNNCNNNNATNAANCCCNCNCGTTCTCCTCTCTCACCTNTCNCCTCATCACTTTACTAATNTNTA
ATNATCNACGCGNTNCGCTTNNANANGTCNNNCANNCNNTGTNTCNGTCCTCNNNCGATNTACTCAGCC
NANNATNNTCCTTAATACNTGTGCTCTGCNNATGTTATCATTTACANGANNNGANTCCTATNTTCTNN
ATTNNNTCTNGNANTNNNTCANNNGATANNTATACCTATATTACTCNCTCTNCAATNTTACAGNTNTA
ATANNNGCNNGACANTNTATGATGNANCGNTATATGTANANTACTCATATCNNCGTGNGCGTTACNA
NNTNCNATATCTCCTCANAANGTNNCTNCCTTTNTCAGTGACACAANATNNGATNNTGNCCGCTANAG
NNGNNTGGTNANNNNGNATNTNAGTATCATTATNTATTANTNGTNCGCTCACATANACGNNGNNCNG
NCGNCTNNNCTNTATTNCATATATNAACGNNTCNCTCAAACANNTAATNNNTAGAGTCC
TNAGCCTATNNGGAGNTNNATNNCAGTGGCGGCCGCTCTAGAACTAGTGGATCCCCCGGGCTGNAGG
ANTTCGGCACGAGGCCCTGACGTGCACATGCGGGACCTGGTGGGCATGGATCACNATTTCTTGCCANG
TGAAGCCANCNANTNGAACGGGGNCCANNNNTANCANNTNNTTCNNTNTTTTGCNNGNTNGCNAGAA
ATNGCAAANGAATTNCCTNCCCANGAAAANGACNGGCNANCNCTNNTNNTGNTNAAGGNNGANCACC
TGANGAATGNCATNAACNTTAANCTGGGGCCANCCCTTAAGAANTACNCACGTATNAGCATGCNNAA
NGACNTTAAGGCTNGNCGGCGCCGGGCTTCTCCTCCCGAACGAGCANAAACCGGTNGACACTCTTGNG
GGGCCCAAAGNACAGGGCGGTCAAGGGAAGGGCCCCGGCTGCCGGGGCGTACCTGTGGGAGGAGGTC
TCNNGACCTCCTTGTGGGGCTCTCAANNGGCCTTTCTTTNTGTGGGAGNGGCACAAAGGTANGTGGCA
CACAANATGGGGCTNTATGCTTNTANAATATTGGTGACACTGTNTTTTTCCANANTNCCACGACTCTAG
CCCCGCTTCTTTTTCNCTNTTTTTTGTGCCCCATTNTTCAAGGGGTANTATGGNGANGGCTTCCCANCCT
GNGGTGGGGNNATTTTTCCCAGGNGNANCCNNCCGGGCNCTTCAAGAAAAAGGNCTTCNTAAANCCC
TCCNNCTGGCNTTTCTTTCCTTTTTTTTTTTTTGGGGGGCCCAACCATTNTTTNTTCNCGNTGGCAGCCT
TTTTTTCCACTTTAAAAACTTNTTTACTTNAANNCCGCCCANAAAGGNGNGNTTCCCNTCNTACCCCCN
TCCAAGGNGGGANGGAAAAAAAAAAATTTGNTNCCGGNTTTGTTTCANCCCCCTCCTTTGGTTNNAAA
CCCCNTTAAATCNCTTTGNNGGGNGAAANNGNNTTGC
TNAGCCTATNNGGAGNTNNATNNCAGTGGCGGCCGCTCTAGAACTAGTGGATCCCCCGGGCTGNAGG
ANTTCGGCACGAGGCCCTGACGTGCACATGCGGGACCTGGTGGGCATGGAACACNATTTCTTGCCANG
TGAACCCANCNANTNGAACGGGGNCCANNNNTANCANNTNNTTCNNTNTTTTGCNNGNTNGCNAGAA
ATNGCAAANGAATTNCCTNCCCANGAAAANGACNGGCNANCNCTNNTNNTGNTNAAGGNNGANCACC
TGANGAATGNCATNAACNTTAANCTGGGGCCANCCCTTAAGAANTACNCACGTATNAGCATGCNNAA
NGACNTTAAGGCTNGNCGGCGCCGGGCTTCTCCTCCCGAACGAGCANAAACCGGTNGACACTCTTGNG
GGGCCCAAAGNACAGGGCGGTCAAGGGAAGGGCCCCCGCTGCCGGGGCGTACCTGTGGGAGGAGGTC
TCNNGACCTCCTTGTGGGGCTCTCAANNGGCCTTTCTTTNTGTGGGAGNGGCACAAAGGTANGTGGCA
CACAANATGGGGCTNTATGCTTNTANAATATTGGTGACACTGTNTTTTTCCANANTNCCACGACTCTAG
CCCCGCTTCTTTTTCNCTNTTTTTGTGCCCCATTNTTCAAGGGGTANTATGGNCANGGCTTCCCANCCT
GNGGTGGGGNNATTTTTCCCAGGNGNANCCNNCCGGGCNCTTCAAGAAAAAGGNCTTCNTAAANCCC
TCCNNCTGGCNTTTCTTTCCTTTTTTTTTTTTTGGGGGGCCCAACCATTNTTTNTTCNCGNTGGCAGCCT
TTTTTTCCACTTTAAAAACTTNTTTACTTNAANNCCGCCCANAAAGGNGNGNTTCCCNTCNTACCCCCN
TCCAAGGNGGGANGGAAAAAAAAAAATTTGNTNCCGGNTTTGTTTCANCCCCCTCCTTTGGTTNNAAA
CCCCNTTTAAATCNCTTTGNNGGGNGAAANNGNNTTGC
TNNANAAAAGCTGGAGCTCCACCGCGGTGGCGGCCGCTCTAGAACTAGTGGATCCCCGGGGCTGCAG
GAANCCGNTGNCNANNCTCAACCANCCATAAAGATATTGGNACCCTTTATCTACTATTGGGNGCTTGG
GCCGGNNTAGTAGGAACAGCTCTAAGCCTTCTAATTCGCGCTGAATNAGGCCAACCCGGAACTCTGCT
CGGAGACGACCAAATCTACAACGTAGTTGTANCCGCACACGCATTTGNAATAATCTTCTTCATAGTAA
TACCAATCATAATTGGNGGATTCGGTANCTGACTTGTNCCCCTAATAATGGGTGCTCCCGATATAGCAT
TTCCCCGAATAAATAATATAAGCTTCTGACTCCTCCCTTCCTTATTCCTACTACTCCTCGCATCCTNTAT
AGTTGAAGCTGGGGCANGAACAGGCTGAACCGNGTNCCCTCCCTTACAGGCAACCTANCCCATGGCA
GGACCTTAAGTANATCTAACCATTTTNTNTTTACACTTAGCAGGAGTTTCCTCAATTTTTAGGAGCCATC
AACTTCATTACAACAATTATCAACATAAAGCCCCCCGCAANGTCACAATACCAAACCCCTNTGTTGGA
ATGANCCGNAATAATNACCGGCCGNACTACTACTAACTTTTNGTTCCCTGGATTANCAGGCGGCATCA
CAATGCTNTTAACAGAACCGGAACCTAAATACAACCTTTTTTCGACCCGGNNGGAGGAGGAAACCCTA
TTNTATTTCAACCCTTTATTCTGATTNTGGGACCCCCCGAAGGCTATATTTTAAANCTTANCNGGGG
TTGGGAAAAAATCCNTTCATATTNGGGACCCTACTTCCTTCNGGGAAAAAAAANAAACCNNTTCNGGA
AATTTTGGGGAANAGGNTTGGGGCCTTTAAANGGCAAANNGGAATTTNTAGGGGTTCAATCGGAANG
AAC
TTGAAACCACTGGACTCCCCGCGGTGGCGGCCGCTCTAGAACTAGTGGATCNCCCGGGCTGCAGGNTT
NAGTCCTTNGGATNTANNNTCNAANNAAGTATTAGTCAAGTATTNGGNNGCACAAGTAATGGGNTAA
CTTTACACTANGAATTGGCGNTNGGGTGGAGGGTGCATGAATCNTTATTCTATTTNATTTTTTNGGATA
NAAGNTNTATCTATTATANATTAANCGCNCTTGCTGACANNNNTGCANAACCATANATAAATTNNANGN
TAACTTTNAGGGCTGGANTGCTTCTCNNNNTNCNGNANATGNCCNNAAGNATTCGAATNGANACCCC
NAANCAGAAAANNTTGNANNGGNCAGGNNGAAATGNTTNTACCTTCGTTGGGNNGANCCTCACCGAA
ATNTTTNACCCTGCAGANNNTTGGGCNATACNGCTNNNTCTANNCNTGTNGNNACANNCTTTNCCNAA
TGCNNTATCTCATCTCTNTNNGTTTCCNNNTTNTNCCTNNNNNTGNCCNTTGCNTTTTTTTTTTTTCCNT
TTCNNNTNGTNGCNTTTTTNACTNTNTCATTCTANNTTTATNGANTTNNNNAATNTCCNANTNNNNACN
ANNNCTCNTTNNACNTCTATTAANTACTATNGANCNNNCTNATCNNANNATGTNNTNNNTTTNATNTC
CNGNCNNCTCTTCTTTCNCNCCNTNTATNNNGCCNANNCNACNNTTNGNNATNNCTTCTTCTTANGNN
NCANNNNANTNCNATTCNTGNATACATTTNTGANNANCTTTATNTTNNGNATACTCTNACNNCAANNN
NNANCAGANNTCCCNTAACTATAANTACACTTTTTTTATATTNTTNACCCGNGGNNNATANAGAATAC
ANACNCTNGACATNCTNCTTGACGTNANGACCGTNNNNACGTTNTTCNACNAGANCCNTTAGANGTNTT
NACNNNTTTNCATAATNTNAANCCCCNTCATGNNTCTGACGANTGCTCTATTATTNNTNANACNGATTT
CNTACNCTAGCNATANCAGCTGANGTTANTCCCTNCCG
AAAAGCTGGAGCTCCACCGCGGTGGCGGCCGCTCTAGAACTAGTGGATCCCCCGGGCTGCAGGCCGCC
ATGGGCCGCGTGATTCGTGGGCAGAGGAAGGGCGCCGGCTGCGTGTTCCGCGCACATGTGAAGGACA
GAAAAGGCGCCGGGCGCCTACGCGCCGTGGATTTCGCCGAGCGACACGGATATATCAAGGGCATCGT
GAAGGACATCATCCACGACCCGGGGCGCGGAGCGCCCCTTGCCAAAGTGGTTTTCCGGGATCCGTACC
GTTTTAAGAAGCGGACAGAGCTGTTCATCGCTGCTGAGGGCATCCACACCGGCCAGTTTGTGTACTGC
GGCAAGAAGGCCCAGCTCAACATCGGCAACGTGCTCCCGGTGGGCACCATGCCTGAGGGCACGATCG
TGTGTTGTCTGGANGAGAAGCCTGGTGACCGAGGCAAGCTGGCAAGAGCCTCTGGAAACTATGCCAC
AGTCATCTNCCACAACCCTGAGACAAAANAAGACGCGAGTGAAGCTTGCCTTCGGCTTCCAAAAAGGT
CATNTTCTCTGCCAACAGANCTGTGGTCGGTGTGGTGGCTTGGAGGTGGNCCGCATTTGACAAGCCC
ATTCTGAAGGCCGGCCGTGCCTTACCACAAGTATTAAGGCAAAAGAGGAACTGGTTGGCCAGGGGTG
GCGGGGTGTGGGCATGAAACCCTTGTCGAGCATTCCNTTTGGANGGNGGCAACCANCCAACACATTGG
GCAAANCCTTTTATTNTTTGCAAAAAACCCCCCCTTGTTGGGCCCGGAAAGNGGGTTTTNANTTGGTTG
NCCCGCCGGAAAAGGNCCGTTTTCNGGGGAAACCAAAAAATGGGCCGGGGAAAANGGANAAACTTA
GGGNTTGGGGGTTTAAAANAAAAGNTTGGTTTTTTTTTNCCAACTTGGAAAAAAAAAAAAAAAAAAA
AAAACTTGGGGGGGGGGGGCCCGGGGCCCCCANNTTTTNT
TTGANAACGCTGGACTCCCCGCGGTGGCGGCCGCTCTAGAACTAGTGGATCCCCCGGGCTGCAGGAAT
TCGGCACGAGGGGACTNGTTCTCTNANGCTGTCAAAGTGGCAGAACTCGAGAAGCGCCTGACAGAGC
TGGAAGCCACTGTACGCTGTGATCANGATGCTCAGAATCCCCTTTCANNAGNTCTNCANGGAGCCTGN
CTCATGTGTACTGCNTNANACNTNGGGGNGANTCNCACTNGCCNTGTGGTTGNTNCTTTCGTCCNAN
TACCCACNATNNTTNCNNNTGGATNNGCNNGTNTTTTCAAGANTNNATGCTNTTCNCNNATTTNNCAT
NNNNGTANAATTTNNNNGGGGGGGCCCCNGAACCCNNATNNTCCAATTATTTGNNNGGNCTNCNTTN
CCATTTNGNCCTTNTTTTNTCTTTGTTCTTNATGTNAATATTNACTTGCCTCCATCANTCNGTCTTGNAN
GATCAAAGTNATNCNTTTTTTGNTTCANNTTTTNGACTCCTNTTGGTATTANCANCGTCAACTNNTTGA
TTNNTTTTGTTNCCTCNTTTNCTATTNNNCNTCNCTTTTNNANTTCNGGGTNTANAANGGATNNATTGT
NNTNAACANCNTGCTTATCTTCTTNAAGGTNGACTTTTNNCCANCATTTGNTTNCNTATNCNTNNCTTT
ACTCNTTTTTGNACNTNNNCTCTCTTNNTCNACTCNATNTNANTCTNNNNGNNNATCTTATCNNCTNTT
TCTNANANTCCNNNGCTNGTATNTTTNCTTTTNNTCACTANTTGTNTNTCNTNCNTNNANNNTAATCTTC
NCTTCNCTANTTTTANTTNTACAATTTTGNAGNNTATGTNCTTNTTTATNACTNTTTAGAGNCTAGTAT
ATTTATGCNNTTTCTTNTANTNNTTNANNNACTNGNTGANTTTTNGNTATATTTTAATNGNTTTNNCN
CGNTCNCCGTANATTTCTCTTNCTTGNTNTAGCGGTNGAGNACTCCNTTANTGGTCTNCGTACGTCTCT
CTCTANCTNCTCNTTTCNNCTCG
AAANCTGGAGCTCCACCGCGGTGGCGGCCGCTCTAGAACTAGTGGATCCCCCGGGGTGGAGGAATTCG
GCACGAGGGGGGCGGTGGCGGCGGTGGCTGGCGGTTCGGCAGTGAGTGCAAGGATCACCATGATTCT
TCAGAGACTCTTCAGGTTGTCCTCTGCTGTTCAGTCTGCAATCTCAGTCTCTTGGAGGAGGAACATTGG
TATTACAGCAGTGGCATTTAATAAGGAGCTTGATCCTGTGCAGAAACTCTTCGTGGACAAGATTAGAG
AATATAGAACTAAGCGACAGACATCTGGAGGACCTGTTGATGCTGGCCCAGAATATCAGCAAGACCT
AGACCGGGAGCTTTTTAAACTTAAGCAAATGTATGGTAAAGCAGACATGAATACGTTCCCTAACTTCA
CGTTTGAAGACCCCAAGTTCGAAGTTGTCGAGAAACCACAATCCTGAAGAAGTAATGTAAAATTGATC
TGGTAATTTGTCCTAGATAGTTGTACAGCTGGCCACAAGTAACAGAATAAACATTTCATAGCTGTCA
AATGTTCTTTTAATTCTGATTTCAAATAAATTATTTGGTGATGTTAGGTAAAAAAAAAAAAAAAAACN
TCGGGGGGGGCCCCGGTNCCCAATTNCCCCTTTNGGGGGGNNGNNNNNGANNNNCNNTNNANGNNNT
CNNCNNANNCCCNANCCCTNGNNCCNNTTNNCNGGCNTTNNTNCCNNNNNNGNNNAAAACNNTNNGC
NTNGNNNCTNTNNCCNTCNNCATNNTGAANNCNNNNNNANNNCNTNNTNNNTNNNNNNCNNGTTNNN
NNANNNNNCNCNNNNNCNNNNCCCNTNTGGNNNNNNNCNCNTNGGNNNNNNNCNNNNCNNCNCT
TNNNNNTNNNCCNNNNNNNGNNNNNNANNNNNNNNGGTGGGNTNNNANNNNAANTNNNNNNNNCN
NTTNCTNNNNGNNNNNNNCNNNNNNNNNNNNNNTNNNNNC
GCCTATNNNGAGNNGNATNACAGTGGCGGCCGCTCTAGAACTAGTGGATCCCCCGGGCTGCAGGAAT
TCGGCACGAGGCTTGTAGTTGTCATCCTTGACCTGGCTAAGCCCTGGGAGTGTTAGTGTAGATCCCATG
GGATCTATGTTGTCTCTTTTATGCCACCGTGGGGCATTTTGTTAATTTGCTACTCTACCTTGAATGGAAA
ACAGGGCCTTGGTCAGAAAAAGAATGCTCTGGACACTGCTAAGGACTTAGAGTCAGCCCATGGTGATT
GTTTGGCTTCTTGNTGTCTGCGTCTTCTTTCCTAGTGTCTGCCTGGCTCTTTANCACAACCTCCCAAGGG
TGGACAGGGGGAAAGTTGGTGGTGTCAAAGGTCAAAACTATGGTGTAGAACAGTGCTCGAGACCCTC
TGTGATCTTTCTAAATGAACAGAAATTGATCCATACCAACGAGGGGGCAACTTGTCAATTTCTTTAATG
TCTCAATATTAACTGGAATGCTGCCTTGNGGGTTCTCACCTGCNTGAATGTTTTGAATTGGATGTNATA
CTGGCTATAAGTCTCCAAAGGAATAACTGACTCAACTATTTACCCTGTTTACTCCAGTAAACAAGTTTT
CCTCATCCCCTCAGNGTAAAAATCCTTCAAAAGGTAGGCGAATTTGCTTCTGGGGTGTTAAAAGCGTA
NGTGGGGATATTTTGAAGGGAGGAAAAGTCNNGTTCNGGCANAAAGGNGTTTTCCCCCNNTAAAANC
TTAAGGTATTTNNCTTGNNTNNGGGTTTTAAACCCCNCTAAAACTTTTGNANGGAAAAGGNTTCCNCC
CCCTTACNAANGAAAAAACNTGCTTNNAAAAANCCAAAANNGGNNTTNGNAANGAAAAAAAAANGG
GGNNAAAANGAAACNTTTNCCCTGGAANNTTNTTCTTTNGGGNCNNGGGNAAAANACANCAAAAAGG
TGAAAAATATTTTTCTTTNTTTANAAAAGGNCANCTACACCCCTTGTGGNAAANNNNNNNNNCACCCC
TATCT
GCCCTATAGNGAGTCGTATTNCAGTGGCGGCCGCTCTAGAACTAGTGGATCCCCCGGGCTGCAGGAAT
TCGGCAGGAGGAAGGTGCCCATNANGCGCTCNGAAATGCTAAGGGATATCATCCGTGAATACACTGA
TGTGTATCCAGAAATCATTGAACGTGCATGCTTTGTCCTGGAGAANAAATTTGGAATTCAACTGAAAG
AAATTGACAAGGAAGAACACCTGTATATTCTCATCAGCACCCCTGAGTCCCTGGCTGGCATACTGGGA
ACGACCAAAGACACACCAAAACTGGGTCTCCTTTTANTGATTCTGGGNGNCATCTTCATGAATGGCAA
CCGTGCCANNGAAGCTGTCCTCTGGGAGGCACTACGCAAGATGGGACTGCGTCCTGGGGNAAGACAT
CCCCTNCTTGGAGANCTGAGGAAGCTTCTNACTTACNAATTTGTAAAACAAAAGTACCTAGACTACAG
ACGAGTACCCAACAGCAACCCTCCTGAGTATGAGTTTCCTCTGGGGCCTCCGATCCTACCATGAGACT
ATCAAGANTGAANAGTGCTACGATTCATTGCTGAGGTTCAGANGAGAGACCCCCTGTNACTTGGACTG
GACAGATTCATGGAGGCCCCGANAANNAAGCGCTNGGATGCTTTNGTATGCTNCTNTCCCCCCATNGC
TTGANAGCACGGGCTGTAGGCGANNAACCCCCCCNTGGGGGAATTNGGNGAATGAANGNNCGTANTC
TTNNnGCCCTTGTGNAGCCTTGGAATGANCAATTNTATTTTTTAACCTGCTGANNNGTGGGGATNAAT
GGAANTGGNGAATTTNTGNTNGAANCCCNTGGTNCCAGAATTNCCCACTTTAACCTTNTTGGGGCCCN
CNATACTNCCCNAANAATAGNCTTTCTCNCCATNATTTTCNTAAAANCNNTTTTCCGGGNCCCCANTN
ATTNGTCCCCTTGNGNGCTNACNAAAACCAANTTCCCNNNCNTC
NCCCNCAGNCAANNNTNTANTNTNNNNNTTTNNTTNNNNTNNTNNNCCNNTCNNNTTNTNTANTNTTT
TTTTTACCTANTCTNTTGGNNNNGNNNNCGNTNTNACCTTTGANNNCACTGGNCTCCCCGCGGTGGCG
GCCGCTCTAGAACTAGTGGATCCCCCGGGCTGCAGGAATTCGGCACGAGGTTGTNANTTACCAATTAC
CTGNGGTATTGGAGAAGACTCTTGAGAGCCCCTTGGACTGCAAGGAGATCCATCCAGTCCATCGTAAA
GGAGATCAGTCCTGGGTGTTCATTGGAAGGACTGATGTTGAAGCTGAAACTCCAATACTTTGGCCACC
TGATGCAAAGAGCTGANTCATGTGAAAAGACCCTGATGCTGGCAAAGATTGAGGGCAGGAGGATCAA
GGGGACGACNTNANGATGGCNTCACCGACACAATGGACATGGGTTTGGGTGGACTCCCACAGTTGGT
GATGGACATGGGAGGCCTGGCGTGCTGTGGTTCATGGGGTCACNAAGAGTCGGACAAGACTNGAGCG
ACTTGAANTTTAACTGAACTGNNNNTACCTCTCCTNCNNTCTTGGANNATANCCNCCTTACNCTCTNCT
TTCCTNAAACTGGANTNTTTGAACCTTTTNCTNGCCCCNGGGCCATTGNCTTTTAAATANTGGGTCCAA
TCTTNANNTGCCCATTATTAGNAAGNGTTTAAAAAGCANCTTTTTCTTNATTTNTTTAAAAANTAAAAA
AAAANNAATTTGGGACCATCTTNNCCCNTTTTCNTNCNNTNNNCNTTCTTTNNCTTCCNATGNTAGNGG
NCNCNTTTNACTCCNCTATNTTCCTCCNCNTTTTCNNNATTNCNNNNANTANNAAACTTACTNACNNCC
CGTGTNCCNTCGCNNTTGTATTTCNTCCGGGGNTCNNCCNTTANGNNTCTCCCNTTNCCCNTCATGNT
ANTNTTNTNTNAGATNTCCTATCTCTNNCTATNCCTCGTANGTCTCCTTCGATTTCAACATNGNTANA
CCCNCTNGNATGTNTTCTNACCTNTCGANGTCCTGCTCGGNNNGCTCTNCNCNCGTATTCTCTCANNNT
GTTNGGCACTCGCACNNANTTNCTACNCACTCTTGNAGGGTTNTNCNTANTNAGCACGCCTNNNTATA
CNNNTCACCTACNANNNTCCTNNCCGNGANTNTGNANNCGNANNTTTTNTGTTCCTCACNNNTNATAT
CTCTGTCGCCNNGGAGTGTNTTAACGACTNTCTCTTGTNCTNCGTTTGNACGCACNCTCCGNNANGNTG
CCNACNCNCCG
NCCCNCAGNCAANNNTTTTANTNTNNNNNTTTNNTTNNNNTNNTNNNCCNNTCNNNNTTNTANTNTTT
TTTTTACCTANTCTNTTGGNNNNGNNNNCGNTNTNACCTTTGANNNCACTGGNCTCGCCGCGGTGGCG
GCCGCTCTAGAACTAGTGGATCCCCCGGGCTGCAGGAATTCGGCACGAGGTTGTNANTTACCAATTAC
CTGNGGTATTGGAGAAGACTCTTGAGAGCCCCTTGGACTGCAAGGAGATCCATCCAGTCCATCCTAAA
GGAGATCAGTCCTGGGTGTTCATTGGAAGGACTGATGTTGAAGCTGAAACTCCAATACTTTGGCCACC
TGATGCAAAGAGCTGANTCATGTGAAAAGACCCTGATGCTGGCAAAGATTGAGGGCAGGAGGATCAA
GGGGACGACNTNANGATGGCNTCACCGACACAATGGACATGGGTTTGGGTGGACTCCCACAGTTGGT
GATGGACATGGGAGGCCTGGCGTGCTGTGGTTCATGGGGTCACNAAGAGTCGGACAAGACTNGAGCG
ACTTGAANTTTAACTGAACTGNNTACCTCTCCTNCNNTCTTGGANNATANCCNCCTTACNCTCTNCT
TTCCTNAAACTGGANTNTTTGAACCTTTTNCTNGCCCCNGGGCCATTGNCTTTTAAANTGGGTCCAA
TCTTNANNTGCCCATTATTAGNAAGNGTTTAAAAAGCANCTTTTTCTTNATTTNTTTAAAAANTAAAAA
AAAANNAATTTGGGACCATCTTNNCCCNTTTTCNTNCNNTNNNCNTTCTTTNNCTTCCNATGNTAGNGG
NCNCNTTTNACTCCNCTATNTTCCTCCNCNTTTTCNNNATTNCNNNNANTANNAAACTTACTNACNNCC
CGTGTNCCNTCGCNNTTGTATTTCNTCCGGGGNTCNNCCNNTTANGNNTCTCCCNTTNCCCNTCATGNT
ANTNTTNTNNTNAGATNTCCTATCTCTNNCTATNCCTCGTANGTCTCCTTCGATTTCAACATNGNTANA
CCCNCTNGNATGTNTTCTNACCTNTCGANGTCCTGCTCGGNNNGCTCTNCNCNCGTATTCTCTCANNNT
GTTNGGCACTCGCACNNANTTNCTACNCACTCTTGNAGGGTTNTNCNTANTNAGCACGCCTNNNTATA
CNNNTCACCTACNANNNTCCTNNCCGNGANTNTGNANNCGNANNTTTTNTGTTCCTCACNNNTNATAT
CTCTGTCGCCNGGAGTGTNTTAACGACTNTCTCTTGTNCTNCGTTTGNACGCACNCTCCGNNANGNTG
CCNACNCNCCG
TNNNNAAAAGCTGGAGCTCCACCGCGGTGGCGGCCGCTCTAGAACTAGTGGATCCCCCGGGCTGGAG
GANTTCGGCACGAGGCTGNAGCCCTCCGNGATGTNTCTGAGGAGTTGANCCGNCANCTGGATGACATC
CTCANNACATACTGCGTGGACAACAGNNANNGGGGCCCAGGTNAGGATGTGGCANAGGGNGAGCCTG
CTGAACCCGAAGATGCANAGAAGNCCNGGACCTATNCCTCANGGANTGGGNAGCCTGAGCCAGATAC
TCCAGTAGTCNNTGNNGAGAAGGAANNCTCCAAGGGGGAGCCGGGCCCGGTCTAGATCCTGTACCAG
NGATGAAGTTTGAGACCGANACCACCCAAAGCCNCNGGATANTAAGAAANNCTANGGTGTGGGAANG
GAAATACCTTGCCCACTTTANNTNCCNCTTAAAGCCTANAGNGGTGGCAGGGAAGGNACTGNCNCN
NCCGNNTGTGGTGTGGCCAGGTTGCAGTNNTNAAAAACCCAAGCTTACTTGGTTGGTGGGCAANGNGG
GCCCCACTTGGCCGGGNNGGGGCTTTTGTTCTCAANTTCACCCTTNCGTGTGTAGCCTTCCANGNCAGG
CTTGANGGNGGGGTCCCANTTGTTTCCGTTGANCCAAATTTTTTTAAATACTTAAAGNTGNTNTTGGTT
TGNTTTTGGTTTCCNAAAGCAANCCCAGGTTTCTTGGGNGCTTGCCCCCNCCCACCCAGGGTCCTTGGG
NCCAAAATNAAAAAGGGGGNCTTTTCCTTGAAATGAAAGGCCCNTTTCCTTTCTTCCCTTGGGGGCNG
GTTTTATTGNCCCATGAACCTTNNTTTTTTGGACCCAAAAACTTGGGGNGGGTTTTNTTAAAAANGGN
NNNCNTTTTCCCTTTAAGGGGGACCCCCCCGGGGGGGGAGGGNNTTTTNTTNAAAANAAACTTTGGGT
TCAAGGGNNTTTGGCCCCNNTTNAAAAACTTT
TTGAACACAGCTGGAGCTCCCCGCGGTGGCGGCCGCTCTAGAACTAGTGGATCCCCCGGGCTGCAGGC
TTTGTTCTGGAGTATGAGGCCTCAGCCCCGGAGGCCCAGTGATGCCAGGGGCTCAGGTGTTCCAGGGA
CTGAGCCGCAGTGGTCCCCAAACCCTGCGTGTGCTTGTGACAAACAAAACCTGTCGGCTTCCTAGCAT
TTACTTTCTGCTTCAGAGGAAGGAAATGGGAATGCTAATTTTTAAAAGACAACTGGATTTTGAGACTTT
ATCTTAAAATGGAAGCTGTTTCTGATTTTCACGAGATCCCACATATTTCAGTGGGGTCATGATTGACAA
TTCTGTATGGGCACCATCCAGCTCAGGACATTTCTTCCCGTAGGCCAACTGAATTCTTTCCCCTGCACA
AAAAGGTTTTACGCTGAGCAGATGCTGTGTCACAGTTGCGGTTATACATTTCTGATAGCCTCTGAATTC
ATCAGGTCCATGAAACAGGAAAAAGTAGACTATCAGTCAGAAAGCCTGGCCTGTTCCCCTACCTTTTG
CATTAATGGGTGGATGTAATTTGAAACAGTATGCCCTTGTGTCACTCAAGTCCAGCTAGTCCAATATCC
CANGTTCAATATTTGAACATCAGCTGATGGAAAAAAATGGGCTTTGGAAATGCTTTATGAATCTNTTA
AATACCCTTTNCATNTCATCACTCAAATTTCTGAACAGGAGTTGGNNAAAAANGCTAANNNCCCNTCCT
AAACCCCTTTTTTTTTCCTAACNGGGAAAAANAATAGCAANCCCCAAGTCNTTCCCTGGGACCTTGTN
NCCTGGTTGGGAAAGGGCNTTNGGTTTNCCCGGGCCCATTTGGGGTNGGGCCCAAAGGCCGGGGNAA
ACCTTGTTTTNAAAATAAACCCGGTGGGNTTGGGGGGGNAAAAAAAAAAT
TNNNNNAAAAGCTGGAGCTCCACCGCGGTGGCGGCCGCTCTAGAACTAGTGGATCCCCCGGGCTGCA
GGCTTTGTTCTGGAGNATGAGGACCTCANCCCCGGAGGCCCAGTGATGCCAGGGGCTCANGTGTTCCA
GGGACTGAGCCGCAGTGGTCCCCAAACCCTGCGTGNGCTTGTGACAAACAAAACCTGTCGGCTTCCTA
GCATTTACTTTCTGCTTCAGAGGAAGGAAATGGGAATGCTAATTTTTAAAAGACAACTGGATTTTGAG
ACTTTATCTTAAAATGGAAGCTGTTTCTGATTTTCACGAGATCCCACATATTTCAGTGGGGTCATGATT
GACAATTCTGTATGGGCACCATCCAGCTCAGGACATTTCTTCCCGTAGGCCAACTGAATTCTTTCCCCT
GCACAAAAAGGTTTTACGCTGANCAGATGCTGTGTCACAGTTGCGGTTATACATTTCTGATAGCCTCTG
AATTCATCAGGTCCATGAAACAGGAAAAAGTAGACTATCAGTCAGAAAGCCTGGCCTGTTCCCCTACC
TTTTGCATTAATGGGTGGATGTAATTTGAAACAGTATGCCCTTGTGTCACTCAAGTCCAGCTAGTCCAA
TATCCCAGGTTCAATATTTGAACATCAGCTGATGGAAAAAATGGGCTTTGGAAATGCTTTATGAATCTC
TTAAATACCCTTCCATTTCATCACTTCAAATTTCTGAAACAGGAGTTGGGAAATAATGCTAAATTCCCN
CTTTCTTAATCCCCTTTTTTTTTTTCCTACTNGGGAAAAAAANATAGTCAAACCCCCAAGTCNTTACCCT
GGGACCTTGTANCCTGGTTNGGGAAAANGGGCTTTTGGGTTTTCCCGGGGCCCCAAATTTGGGTNGGG
CCCCAAANGGCNGGGGNAAACCTTNTTTNAAAAATAAAAACCCNGGGGGGTTNGGGGGGAAAAAAA
AAAAAAAAAAAAN
AAAAGCTGGAGCTCCCCGCGGTGGCGGCCGCTCTAGAACTAGTGGATCCCCCGGGCTGCAGGAATTCG
GCACGAGGGGACAAGTTCTCTCAAGCTGCCAAAGTGGCAGAACTCGAGAAGCGCCTGACAGAGCTGG
AAGCCACTGTACGCTGTGATCAGGATGCTCAGAATCCCCTTTCAGCAGGTCTGCAGGGAGCCTGCCTC
ATGGATACTGTAGAGCTGTTGCAGGCGAAGGTGGGCGCCCTGGACCTTGCAGTTTTGGACCAAGTGGA
GGCTCGGTTACAGAGTGTGCTGGGAAAAGTGAATGAGATTGCCAAGCATAAAGCTTCTGTAGAGGAT
GCAGATACACAGAGCAAGGTGCACCAGCTGTATGAAACCATACAGCGCTGGAGCCCCATCGCCGGCT
CCCTTCCTGAGCTGGTACAGAGACTTGTCACCATCAAGCAGCTGCATGAACAAGCCATGCAGTTGGT
GAGCTTCTGACACACTTGGATACCACGCAGCAAATGATTGCTTGTTCCCTCAAGGACAATGCCACCCTC
TTGACTCANGTGCAGACGACGATGTGTGAAAACCTGTCCACAATTGAGGGGAACTTTGCCAACATTGA
TGAAAGGATGAAGAAACTGGGAAAGTGAGCACCTTTGGGTGATGGANGACAGGGGTAATTCCTTACC
CCTTTGACTNCATTAATACCTTACACAGAGNTTNCCCNTTAATTGNAACTTNTTGCATTCCANTTTGAC
CTTGGGGGCAAGGGCTTTNTTGCATTGGGGGGCTTACNCCCCCTTCNTTTNAAAANTAATGGGAAGCT
TGGGGAAANTTAAAANGAAGGGNCTNCCCCTTTAGGCCCNCANGGATTTAAGGACCTGGGCCCAAAC
CCAATGGCCCAGNTTTCTTGGCCCAATAANTGGT
AAANCTGGACTCCACCGCGGTGGCGGCCGCTCTAGAACTAGTGGATCCCCCGGGCTGCAGGCCGCGCC
GCCGTCGTCTCCAACGCCAGCGCCGCCTCTAGCTCGCCGAGCTCCAGCCGAAGGAGAAGGGGGGTAA
GTAAGGAGGTCTCTATACCATGGCTCGTACAAAGCAGACTGCCCGCAAATCGACCGGTGGTAAAGCAC
CGAGGAAGCAACTCGCTACAAAAGCCGCTCGCAAGAGTGCGCCCTCTACTGGAGGGGTGAAGAAACG
TCATCGTTACAGGCCTGGTACTGTGGCACTCCGTGAAATTAGACGTTATCAGAAGTCCACTGAACTTCT
GATTCGCAAACTTCCCTTCCAGCGTCTGGTGCGGGAAATTGCTCAGGACTTCAAAACAGATCTGCGCTT
CCAGAGTGCAGCTATTGGTGCTTTGCAGGAGGCAAGTGAGGCCTATCTGGTTGGCCTTTTTGAAGACA
CCAACCTGTGTGCTATCCATGCCAAACGTGTAACAATTATGCCAAAAGACATCCAGCTAGCACGCCGC
ATACGTGGAGAACGTGCTTAAGAATCCACTATGATGGGAAACATTTCATTCTTTAAAAAAAAAAAAAA
AAAACTCGNGGGGGGGCCCGGTNCCCAATTCGCCCTATAGGGAGNNNNNNNAAANNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNN
GCCCTACTGGAGCTNCACGGCGGTGGCGGCCGCTCTAGAACTAGTGGATCCCCCGGGCTGCAGGAAAT
TCGGCACGAGGAGGANNTTGNCNTCCTATNCNTATANNCNNNCTCANTAACCTTNTGNNCNATTTAAN
GAAAGTNTTTGTGGTNTCTTCCCCATAAAGGAATTTACACAACCNCCCNGGCCTGTGCCNGTGGATCA
TAAAAAAAGTGCCCNTTNNCCNNTNGAANGTANTTAAAAAAACCAAAGGGGGGGAAAAAACNNNCN
AAGGCCNAAAGCCTTNNATTGGGGGCCACCCGCCNGGGTGGCNAAAATCCCCTNTGGCCGGGGCNTT
TNTGGGCCNCTTACGGNGCNTAAAAAAGNCCCGAATNTGGNNCCCTGGTNAAGGTTTGNATTANGNT
NTTGGAACCCTTNCCCTGNAANANACNCGGTTATCCNGGCCCCCGCNTGNNATTGGANTCNGTGCCTC
CCATCCTGGGAAGGGGGGTGTTTCATTTACCATTCCNTCCNGGCCTTATTTGAGGGAAANGCCCCNNG
GNAAAATGNTTTNCNATNTTAAACACATTTTTTTATAAGGGCCCCCGCATTCNNGAGGGGTTTTTAAA
GGGGANTANCTTGNTTTTGCCCTGNGNTGGGNNTTGGNCNTTACTAAAAAGCTTCCAAGGGCCCCCCT
TTTTTGACCCCCCCTTTTTTCTTTTGGGCNANAANGNNCAAAANAGCCTTTTAAAAAACCNGGGGGGA
AGGGGGGGAACCCNTNGNNTTTTGGGGGGGTNTTAAATTGNCCCCNCCCCCNCCCAAGGNGGTGGTN
GGGGGNTTTGCCNNNNACCGGNCCCCCCNCNNTTTAAAAANNNANNAAGGGGGGCCNTTTTGGGGGN
CCNAAAANTNNNNANCNTTTTTTTTTNCCCCCTTTTTTTNGNNTNANNTTTNGGGGGGGGGNCCCCCC
CCGGGGGGGGGNCNNNAAAANNNNNNNNNNNNNNAAAAAANNGNNNCNCNNNAAAAAAAAAANNN
NNNNNNNNNNNCT
GCCCTATANNGAGNNGTATTACAGTGGCGGCCGCTCTAGAACTAGTGGATCCCCCGGGCTGCAGGAAT
TCGGCACGAGGATTGCAGCAAATCATGAAAGGTAACTTCAGTGCATTNATGCAAAAGGAGATATTTGA
ACAGCCAGAATCCGTTTTCAATACCATGAGAGGTCGAGTGAATTTTGAGACCAACACAGTGCTCCTGG
GTGGCTTGAAAGACCACTTGAAGGAGATCAGACGATGCCGAGGGCTCATTGTGATTGGCTGTGGGACC
AGCTACCACGCCGCTGNGGCCACGCGGCAAGTTTTGGANGAACTGACCGAGCTCCCCGTGATGGTTGA
ACTTGCTAGTGATTTTCTGGACAGGAACACACCTGTGTTCAGGGATGACGTTTGCTTTTTCATAAGCCA
GTCAGGGGAGACTGCAGACACCGTCCTGGCACTGCGCTACTGCAAGGACCGCCNAGCTCTGACCGTGG
GCGTCACCAACACCGTGGGCAGCTCCATCTCCCGCGAGACGGACTGCGGCGTCCACATCAACGCAGGG
CCGGAGATCGGTGTGGCCAGCACCAANGCTTACACCAGTGCAGTTCATCTCCTTGGNGATGTTTGGTTT
GATGATGTCTTGAAGACCGCATTTCACTGCCNAACAGAAANGGCGAGAAGATCAATNCATTGGCCCTG
GAATTCTTCTACCCCNAGTCTTATTCNAANGAAGGTGCCTNTCCCNTTGGTATTGAAGAANATTGCANC
NAANCNTGGNCCCTTGGTNNCNTNTNCACCACCAAAAGGTTCANCNTTTTTGGTCCATTGGGGGCGNA
AGGCTNACAANNTATNCCCACATCGGCGTGGNAAAAGGAAGCCCCCTTGAAAAATTTAATNGAAAAT
NNNCCCTATTTGCCCTTTAAAAANGGCATTCCTNGGNTTNGNANAAANTTAAAACNNCGGGCCCCCCT
TGNNCCTTNNTTTNNATNAAGCCCANAGCCTTC
AAAACTGGAGCTCCCCGCGGTGGCGGCCGCTCTAGAACTAGTGGATCCCCCGGGCTGGAGGANTTCGG
CACGAGGTTGGCAATTACCAATTACCTGTGGTATTGGGAGAAAGACTCTETGAAGAAGCCCTTGGANT
GGAAGGGAAATCCTTCNATCCNTTCTAAANGNAANNAANCGNNGGTGGTCNTTGGAANGNCNGNNGN
TNAANCTNAAACNNCNATACTTTGGCCNCCTNNNGCNAAAANCNNACTNATGNGAAAAGAGCCTNNT
GCNNGCNAANANTGAGGGCCGGANGANAANGGGACCACCGAAGATGGCNTNCCNACACCATGGNCC
TNGGTTTGGGTGGACNTCCCAAATTGGTGAATGACACAGGAGAGCCTGGCGTGCTCTGGNGCATGTGG
GTCACACAAGAGACCACACAACATGAGCGANTGAGCTGNGCTNNGCTGNGATTACCTCTCTTTCACTG
TGANATACCCCTTACNTCTCTTTCTCNACTGANATTGAACTNTTTTGNNCACGTNATGTGTTAATATGG
GNCATCTTANNGCCNTATATAGGGNNNAGACTTTNTTATTTTATAATATAAAAAATTGGGCATTTTAG
AAAAAAAAAAAAAAAAAACNTCCGGGGGGGGGGCCGCCCCCCAATNTNCCCTCTTTTTNGNGGGNNNT
TTANNNNNNNNNNNNNNNNNNNNNNNCTNNNTNNNNNNNNNTTNNNCCNNNNNNNNNNNNNNTTTN
NNCNNNNNNNNNNNNNNNNNNNNNNTNNNNNNNNNNNNNNNNNNNNNCNNNNNNNNNNNNNCNNN
CNNNNNNNNNNNNNNNNNNNNNNNNNNNNNNNNNNNNNNNNNNNNNNNNNNNNNNNNNNNNNNN
NNTTNNNNNNNNNNNNNNNNNNNNNNNNNNNNNNNNNNNNNTNNNNNNNNNNNNNNNNNNNNNNN
NNNNNNNNNNNNNNNNNNTNNNNCT
AAAACTGGAGCTCCCCGCGGTGGCGGCCGCTCTAGAACTAGTGGATCCCCCGGGCTGCAGGANTTCGG
CACGAGGTTGGCATTACCAATTACCTGTGGTATTGGGAGAAAGACTCTTTGAAGAAGCCCTTGGANT
GGAAGGGAAATCCTTCNATCCNTTCTAAANGNAANNAANCCNNGGTGGTCNTTGGAANGNCNGNNGN
TNAANCTNAAACNNCNATACTTTGGCCNCCTNNNGCNAAAANCNNACTNATGNGAAAAGACCCTNNT
GCNNGCNAANANTGAGGGCCGGANGANAANGGGACCACCGAAGATGGCNTNCCNACACCATGGNCC
TNGGTTTGGGTGGACNTCCCAAATTGGTGAATGACACAGGAGAGCCTGGCGTGCTCTGGNGCATGTGG
GTCACACAAGAGACCACACAACATGAGCGANTGAGCTGNGCTNNGCTGNGATTACCTCTCTTTCACTG
TGANATACCCCTTACNTCTCTTTCTCNACTGANATTGAACTNTTTTGNNCACGTNATGTGTTAATATGG
GNCATCTTANNGCCNTATATAGGGNNNAGACTTTNTTATTTTATAATATAAAAAATTTGGGCATTTTAG
AAAAAAAAAAAAAAAAAACNTCCGGGGGGGGGGCCGCCCCCCAATNTNCCCTCTTTTNGNGGGNNNT
TTATNNNNNNNNNNNNNNNNNNNNNNCTNNNTNNNNNNNNNTTNNNCCNNNNNNNNNNNNNNTTTN
NNCNNNNNNNNNNNNNNNNNNNNNNTNNNNNNNNNNNNNNNNNNNNNCNNNNNNNNNNNNNCNNN
CNNNNNNNNNNNNNNNNNNNNNNNNNNNNNNNNNNNNNNNNNNNNNNNNNNNNNNNNNNNNNNN
NNTTNNNNNNNNNNNNNNNNNNNNNNNNNNNNNNNNNNNNNTNNNNNNNNNNNNNNNNNNNNNNN
NNNNNNNNNNNNNNNNNNTNNNNCT
NAAAAGCTGGAGCTCCACCGCGGTGGCGGCCGCTCTAGAACTAGTGGATCCCCCGGGCTGCAGGAATT
CGGCACGAGGCTGGAGCCCTCCGTGATGTCTCTGAGGAGTTGAGCCGCCAGCTGGAAGACATCCTCAG
TACATACTGCGTGGACAACAGTCAAGGGGGCCCAGGTGAGGATGTGGCACAGGGTGAGCCTGCTGAA
CCCGAAGATGCAGAGAAGTGGCGGACCTATGCCTCAAGGAATGGGGAGCCTGAGCCAGAGACTCCAG
TAGTCAATGGTGAGAAGGAAATCTCCAAGGGGGAGCCGGGCCCGGACGAGATCCGGACCAGTGATGA
AGTCGTAGACCGAGACCACCGAAGGCCACAGGANAAGAAAAAAGCCAAGGGTCTGGGAAAGGANAT
CACCTGCCCCACTTTAACTACCTNTTAAAGCCTATAGTGGTTGGCANGGAAGGGGACTTGCCAAANCC
GAGTTTGGNGGGGCAGGTTACAGTCTTCAAAANCCAAGTTTACTTGNTNGTGAAGCAAGGGGGCCCCA
CTTGGCCAGGGTAGAACTTTTGTTTTAANTTAACCGTCCGTTTGTANCCTTCCAGGCCAGGTTGAGGGG
GGGTCCCATTGCTTCCTTGACCAAATNTTTTAAATACTTACAGTGGNTTTGGTGGGTNTGNTTTCGAAA
GCAANCCCAGGTTCTNGGTGCTGNCCCCNCCANCCAGGGNCCTGGNCCAAAAAAAAAGGGGNCCTTT
CCTGAATGAAGGCCANTTCNTTTCTTCNTGGGGCAGGGTTTNTTGNCCCTTGAACCTAATTTTTTGGAC
CCAAAAACTNGGGTGGGGTTTTTTAAAAAGGTAACNTTTTCCTTTANGGGAACCCCCCGGGGGGGAAG
GCTTTNTTTGAAAANAAACTTTGGGTTNAGGGGTTTTGGCCCCNTTTAAAAACTTT
TTNAACAAAACTGGAGTCCACCGCGGTGGCGGCCGCTCTAGAACTAGTGGATCCCCCGGGCTGCAGGG
TCCCCTCCCCCCTTGCCCCTCCAGAGAGTGATGCTGGAGCGTGAGTTGATTTTCTTGGCTCGATCTGTC
CTTTCTGAGCTCTAGTCATAGCACTTTGGAGAGCATCCTGGGCACGGTGGTGGTGCAGCTGCAGCCTGC
GGCGGTCCTTAGCCGCAGGGTCTTTGTTTCACAAAACCCTCTGGTACTGTTGTGGTTTTGTATTCTTCCA
GTGTTCTCTCTCTCTGTCTCTCCTAACTATTCAAAAGTATCTTTGCTCTTTTGCCCTTTCCTTTGACCTTC
TGTTCTAATCCTGCTTTGGAATTAGCCGGTGTGGGGAGAGGTACACCATGTCGGANAGAANAAAGAGT
GCCTGTTACTGGAAGTGTTTCCTATTCACGTTTATTTTGCTCAGTTCTTTTTATTTATGCCCTTGGGGCC
CAGGTACTTGGGCAGCCAGCTAACCTCCTGGCTAGNGAAGGAGACCAGGCNGAAAGTGAACACCAAG
GAAAGGTCGNGTGTTTNTANAACTCCAGCAGANACCAAGCCCTGCTGGAAATTTGCAAGGCCANACC
AAAGGGCCACACAGTTGGTCATAAGNGTAAATATTTTGNNCAGNGAATAAATGAATGGTTTAAAAGG
AAAAAAAAAAAAAAAAAAAACCTNNGGGGGGGGCCCGGGNNCCAAATTTCNCCCTTAAANGGGGNNN
NNNAAAANNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNC
TTGAAAACAATGTGGAGCTCCACCGCGGTGGCGGCCGCTCTAGAACTAGTGGATCCCCCGGGCTGCAG
GNNTNAACCTGNAGNANTTNAGCGCTCCNGCCCCAGGCTANTCAACACAGCNNGGGTCTCTCCTCCTG
CTGNNGNTCTCTGCCCTCCACATCCTCATTTCTTATCCTGCTTTNCGGGGCCACTTTGGACAAGTCCTG
GTGGACTCTCCCCGGNAAGGANNCCCTGAATCTCTGGTACGACTGCACANGGAATAGTGACAACAAA
NCGTGGGCCTGNANAAACGTCAGANAAAAGGGCTTGGNTGAAGGNAGTNCAAGATCCTCATGGNGCT
TTCCCTCATNCTNTGNTGCNTGTCCTTCATCCTGTTNATGTTCCCANCTCTACACCATGCTGGCGAGGA
GGGCCTATTNTATNCCACTGGCTTNTTGCCANNATTNGCNCCANCCNTGGCAGTGTTTACCGGGNGCN
CTNNTNTACCCCATTTCATGCCCGAAANAAAATTNCTGGCNNNNCCCGCCCGTAGGGGGGGCAANCNT
NTGGGTAANTGGCTTCCCNCCNTGGCCCTTGGGGNGGNCCCTTNNNCCCNTTNGCCCCTGGCCNAGCN
GGNAATTAATCTAANNNTCCCCACTNNCCGNNAAACCGGGNAATTNNAGGGGNTNCGCCCTCCCCTTT
GGNTTTNCCCAANACNNAATTTCNNCGCCCCCTATNCNTCNTNANCCCCNGAAAANCCCCCAANCCCT
TTTNTTCCTTTTTTTNAATTTTNAAANTANAAAAATANAATTTGTNNNNCCCCCCCGGAAAAAAAAAA
NAAAAAAAAANNTTTNNGGGGGGGCNCCCCNCCNCCCTTTNTCCCCTTTTNTGGGGGGGCGTCNTTAA
ANAACNTTNCCNCNGNNNTGCNCGGCCAACTGNCNAATTTNATTTATTTTATCCTNCAAATCTTTANAC
CTCTNCCTATCATCCCNCTGTCATCNCTNANNCNNTCNTNCGCCTCTNTCNNTNNTNNCANNTANTANA
CATTACTTNNTGTTATTTCNNTTTANTCNACCNTNNTCNNCG
TNNNACAAAAGCTGGAGCTCCCCGCGGTGGCGGCCGCTCTAGAACTAGTGGATCCCCCGGGCTGCAG
GAATTCGGCACGAGGCTTCGGTAGCGACCGCCTCTCTACGCGGGTGTGAGCTTCGCCAGCCCCTCCCC
CAGGAGACCGTTGCAGTCGGCCAGCCCCTGCTCCTTGGTAACCATGTGTGACCGAAAGGCCGTAATCA
AGAATGCCGATATGTCGGAGGAGATGCAACAGGACTCGGTGGAGTGTGCTACTCAGGCATTGGAAAA
GTATAATATAGAGAAGGACATTGCGGCCCATATCAAGAAGGAGTTTTGACAAGAAGTACAAGCCCACC
TGGCACTGCATCGTGGGGAGGAACTTCGGTAGTTATGTGACACATGAAACCAAACACTTCATCTACTT
CTACCTGGGCCAAGTGGCCATTCTCCTGTTCAAATCTGGTTAAAAGCATGGACTGTGCCACACACCCA
GTGATCCATCCAAAAACAAGGACTGCAGCCTAAATTCCAAATACCAGAGACTGAAGTCTTCAGCCTTG
CCTAAGGGAACATCTNCATCTTTGAACCTTTGNTGNGTTTTGTACAGGGCATTCTCTGTACTAGTTTGN
GGGTATAAAGCAATTAGTAAAACAGCTTACATTTGGATTTATTTTCTATTCCATACGTTTTNTGNCCCA
TTGTTTTTTCTNTTCAAAATCCATTCCTTTAAAAAAATAAATTTGGTTGGAAGTGGGCGTTNAAAAAAA
AAAAAAAAAAAAAATTTGGGGGGGGGGNCCCGGGNCCCCAATTNNCCCTTNANNGNGNNNNNTTNTA
AAANNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
AAAAGCTGGAGCTCCACCGGGGTGGCGGCGGCTCTAGAACTAGTGGATCCCCCGGGCTGCAGGAATTC
GGCACGAGGCTAGATTGGCCTTTNGACGATGGAGCGCCACCCCCTAANCNNATTGNATATGATNGGCT
ANNCCTACTAAAANCCANNTTNCGTGAANAACCNGGTTGCTGTGNTGNAGTGCNTTGNGTNGCANGA
TTGGNAAGGGCNCCTGTGCTACTTGCNCTTGCTTTGATTGAATGTGGAATGAAGTACGAGGATGCAGT
TNAGTTTATAAGACNNAAGANAANGGGAGCNTTCNNTTNCANACNNCTGNTTTACCTGGANAAATAC
CGACCTATGATGCNATTNCTCTTCANAGATACCANCGGGCATTGCTGNGTNCAGGTAAAATGAAGTGT
ATACGAAGGCTGACTTGATTGTAGCATTTACAGGGAACTCTTGGTACGTGGAAATGNGAATCTGGAAT
CTTANCTGTNTCATNAAAGTANTGATGGATTCNGTACTCCTCANCCACTCTNCTAATGATTGTAAAAAA
GCAAACACNANTCCCTNTATAACGNGAATNAAATGTNTAAGAAANGANNATGAGAANGATTATAGGG
GATTAATTNACTGANAGATGATTNTTGCTTCTAGNGCNGGAGTTTGAATTTCTGNCAGGATTGAATTTA
TTTCAAAANCTGCTGTCTTTTTTAACTTTCTCAAAATAGGTNTCTAANGAAANCCANCAGAACATGATC
CTTTCAAAAGCCTCTGTTTGGGGAGCAGACTTTCCTTNTGCTNGGCNCATAAAATTNCCNTGCGGGGG
GAATTTTTTTNTTTTTNNNGGAAACCGNTTTTTNNNNNNNNANAACNTTTTTAANNCCCTNGANCCCTN
TTNTNAACCTTGGNACCNTTTGGNAAANAATNAAGGTTTTTNGNGNNNNNNNNNNNNNNNNNNNNNN
NNNNNNNNNNNGGGGGGGGGNCNNNCCCCCN
GCCTATNTGGAGGTNCATCNCNGTGGCGGCCGCTCTAGAACTAAGTGGATCCCCCGGGCTGCAGGAAA
TTCGGCACGAGGTTTCTTGGTCCATCAACCTAGCCTCTAAGTGGTGTGAGCGGCCTGAGTGGGTGCACT
TGGACAGCCGGCCTTTTGCCTCCCTGAGCCGTGACTCAGGGGCCGCCTGGGTCTGGGCATCGCTCTGC
ACTCTCCCTGCTATGCCCAGGTACGGCGGGCACACCTGGGATATGGCCAGAAACTAGTCTGTCTTGTG
CTGGCCATGGGGCTGCTGGGCCCCCTGAACTGGCTGGGCTACCCCCCTCAGATCAGCCTTTTCTACATC
TTCAATTTCCTCAAGTACACCCTCTGGCCATGCCTGGTCCTGGCCCTCGTACCCTGGCTGGTGCACATG
TTCAGTGCCCAGGAAGCACCACCCATCCGCTCTTCTGACTCTGGTGCCTNCTGCACCCACCTNCCCTNA
CAAAGCCCATGCTCTGTGACCTCCACCCTTGGGAGGCAAGNTCCGTCCACTTCCAGCCCCCAACAACC
CTGCTCATGTTGAATTTTCTACTTCTNCCACCACCTGGTCACAAGCCCCAAAGAGGGGCCTTTTCTCTT
CCAGGAAGGNCCTTCTGGCTTTCCTTCCAAATCCCCAAAGAACCAACGCACNCTNCAGAACAACGGAT
TTCTGTAACACTGGGAANGGGCTCGGAACAAACCCCAATAAAGCCCTTTCACACCCNCCCNNAAAAA
AAAAAAAAAAAACAAAANNNCCCCCGGGGGGGGGGNGGNANANAAATTNNCCCCTAAAANNANNGN
NCCCTNAAANNCCCTTTTTTTTNNNNNCCCCCCCCCCCCCCCCCCCNCCCCCNNNGNNNNNNNNNNNN
NTNNNNNNNNNNNNTTTCCTTTNTAGTNNNNNNNNNNNNNNNNNNNNNCCNCNNCCCCCANNNNNN
ANNNNNNNNNNNNNNNNNNNNNANNCCCCTTTNT
NCNNNATCTGGAGCTCCACCGCGGTGGCGGCCGCTCTAGAACTAGTGGATCCCCGGGGCTGCAGGAAT
TCGGCACGAGGNCCCACCTGNACNTGNAGGCCANNCACTCNAANATCCTCANGNACNTNNAGNCNGG
CNCATACNNCCGGTGNNTNNTNGTNAAAATNNTCCTCATNCTGATTGCNNNCTTNATNATCTTTGAGN
ACTNCCTTGCCTGAACCCTNNNNACTCTNANGCAATNCGTGGGGGTNNGGAANCNTNANNGNAGAGC
NGGNNNCCATANCTGCNNCNGANCCTGANCNGGGGGNTCCGNGAGAANGGCCCCNTTTCCTTGTNAC
TGCTAATACATGACCNCTGNCCTCTGTCCNCCACTCCTTGCCTCTGGCCACCCATCCCTCACCTGACCA
CCCTACNGCCCATGAAACNCACNCGGANNNNGATTNNGACNCTGNTNTNAAGTGGTTNNAANGGATC
ANAAGCCAACTGATGNNCTNNNNGGNNANANTGTCTNTACCTNGAGATNTTTATAAACCCTCCCNAAC
CTGTCNCAGACGGGAACTTTGGCTACANAGNGGAGATGATGCCTTTCCCCACATTNTTGANATNAATG
ATANGANGGGAAACNGCCCACATGNACCAAATNTTCCCATCNNGGTTAGAATNNGANACTATTATTCC
TCTCTTTACCCACATGAGGCACCGGANCCCCTGAGCCCCTNNGGAGGGACTGCACAANCCCCANAATT
NGGGCTGGCAAAATGAACTTTTAATTCNGGACGAAACGTGCCNGTCNCCCCTGTTTTTGTTCCCAAGTT
ACGGGCCTTTGACTGGNCAGGCCTTCNATTTNCTGGGNCATGGNAAAAAAATTTNCCCCTTTGGGGGGG
GGGNCCCNGAANCCCCAANAAACGCCCNTATAAAAACNNNNNNNNNNNTTTAAGNNGNNNNNNTNN
CCCTGGGTTTGNTNTTNCCC
GCCNATANGGAGNCGNATNACAGTGGCGGCCGCTCTAGAACTAGTGGATCCCCCGGGCTGCAGGNAN
AAATTGNNAANGCTGNTGAGCTCTAACAGCACAGACCTNCCACTGANCATGGNGTGCTTCATGAATGA
CAAAGAACGTTCTTGGAAAGANNANCAGGGCCCANTTGGAAAANCTTNGGNCTGNCTANNTGAANAN
AATAAAANTNCCCNTTNNTTTGTGGANGGACCAANNNNCACTTNAANNAAAAGGANGNNANGNTNGNC
AAAANACTNGANGNCCNNNCCNAANTCCCACCCGNAAAGAAAAAAANCNCCAATTCTTTGGAAAAGA
ANNCCNNNCCACCCNNNANNCCAAANAAGCCNTGGCCCAAGGGTGNGCCCCTNCNTTGCNATCCNTT
TCCCCACATTTAAAGGTATAGANAATTTTGTCACTGNTGCNGATTTCTTTTCCANTATCTNTGGNNNNN
GGANCCCGATTTCAACAAATNCNCTANANGGGTTCCATGAGAATGTGNNTATATANAANCCCACCTGT
GCCTTTTTTCGACAAAATTTTCTCGCTTCTCNTAAAANNGGGGCCCTTTTTGNGCTCGGNNNCCTTT
ATTNCTTGTCCNTCAAGGAGGTTNCNCNATTNTNCGANTCNCNAAATAATGNNCCCCTCTNTANTTTA
NAAAANATGTTTCCACCCCCAAGGNNGGNGGGAACNANNTCTNCCTCGGNGNAGANCTCTAAAGNAG
CTGACGGNGGATTCCCCCCNGGNGTTNTNTTTCCNTCTTTTNNTTACNNGTNTATCGGNNGGGGGAAN
AATACCCCCCCNGCCCGANNAACAAACGTGGGTTTTCTTTNTTTTNCCCTCCNTTGGTCNTTTNTGTAA
AATNATTCNGGCCGCCCNTCAANAAATNTCCCTGCGGGGTCATAAAATTTTCATCNCNNTANNNNCAC
AANGAATNCATGTTTCCCNNCCCCNCCCGTTCGNAAACCCAGATTGGTNNTTATAANCCNTCCGNGNN
CGTGCCCCCG
GCCNATANGGAGNCGNATNACAGTGGCGGCCGCTCTAGAACTAGTGGATCCCCCGGGCTGCAGGNAN
AAATTGNNAANGCTGNTGAGCTCTAACAGCACAGACCTNCCACTGANCATCGNGTGCTTCATGAATGA
CAAAGAACGTTCTTGGAAAGANNANCAGGGCCCANTTGGAAAANCTTNGGNCTGNCTANNTGAANAN
AATAAAANTNCCCNTNNTTTGTGGANGGACCAANNNCACTTNAANNAAAAGGANGNNANGNTNGNC
AAAANACTNGANGNCCNNNCCNAANTCCCACCCGNAAAGAAAAAAANCNCCAATTCTTTGGAAAAGA
ANNCCNNCCACCCNNNANNCCAAANAAGCCNTGGCCCAAGGGTGNGCCCCTNCNTTGCNATCCNTT
TCCCCACATTTAAAGGTATAGANAATTTTGTCACTGNTGCNCATTTCTTTTCCANTATCTNTGGNNNN
GGANCCCGATTTCAACAAATNCNCTANANGGGTTCCATGAGAATGTGNNTATATANAANCCCACCTGT
GCCTTTTTTCCACAAAATTTTCTCGCTTCTCNTAAAANNGGGGCCCTTTTTGNGCTCGGNNNCCTNTTT
ATTNCTTGTCCNTCAAGGAGGTTNCNCNATTNTNCGANTCNCNAAATAATGNNGGCCTCTNTANTTTA
NAAAANATGTTTCCACCCCCAAGGNNGGNGGGAACNANNTCTNCCTCGGNGNAGANCTCTAAAGNAG
CTGACGGNGGATTCCCCCCNGGNGTTNTNTTTCCNTCTTTTNTTACNNGTNTATCGGNNGGGGGAAN
AATACCCCCCCNGCCCGANNAACAAACGTGGGTTTTCTTTNTTTNCCCTCCNTTGGTCNTTTNTGTAA
AATNATTCNGGCCGCCCNTCAANAAATNTCCCTGCGGGGTCATAAAATTTTCATCNCNNTANNNNCAC
AANGAATNCATGTTTCCCNNCCCCNCCCGTTCGNAAACCCAGATTGGTNNTTATAANCCNTCCGNGNN
CGTGCCCCCG
AAAAAGCTGGAGCTCCACCGCGGTGGCGGCCGCTCTAGAACTAGTGGATCCCCCGGGCTGCAGGAATT
CGGCACGAGGCCCGTCCCAGCTCCAGCTGGTGACCAGAAGGATGTGGATGCTTCAGAAAAGAAGGTC
CCAGAACAGACCCCTGAGCCTCCCCAGCTGGATTCACAGCACCTGTGATCATTCCAGGACCCCAACAC
TCAGGGTTCCTCCCAAGGCTGATGGGAGGGGGGTGGAGTGTGACAATCAACCTGCTCTGGGGACCCGG
CCACACTTGTGGCCTCCTGTGGGAGGGGGAACAGGCTGGCTTTCCTTCTCATACTGCTGCCCAGGCC
CTGTATCTGCCACCTCAGTCTCCACAGCCTTCCAGTCTGNGGTCCCCTCTTTCTGGTCCCCTCCAAGGG
ACTTGCCTTNTCGTATCCAGTTGAATGCTCACTGGCCTTAACTTTCATTGANGGAAGCCCCCTTNTTTC
AAGGAAGGGTCTGCTCACACCTGCCTTTNTAAGTTGGTTTGGCCTTGACNCCAAGGTGGGGGCCATTC
AAGATTCCGGATTCCCACAAGTTGGTGAAATCCTGCCTGGTGGTTTTGGGCCCAATTTCCGTTGGTCT
GGNGGGGCTTTAANGGGCCCCTTAAACACCTTTTTTTTAAATTTTGGGGGGACTGGGCNATGCCCTNA
AANAAAGNGGCTTNCCCCCCAAGNTGGGGTGGNAAGGGCCAAAAAAAAANCCTTTGGATTNGTTNAA
ANAANTTTTGANNCCCAGGAANCCCTTGACTTTTCCTGGACCCCTNGGNTTTCAAACCATTTTNAANCC
CCCTTTCCTTGAAGGGAAGGGGTTCCTAAACCCCTTTTNTTTCCAAATTCCCAAAAANGGAANAAAAN
AAATTGGNCCCCCCCCGGANGGAATAAGGGTTNCCCACCCCCTTAAACCNTTTTNGGGGTTTTNANNN
NNNAAANGGNTTCCCTTTTTTTTNT
TTNAACACAGCTGGAGCTCCACCGCGGTGGCGGCCGCTCTAGAACTAGTGGATCCCCCGGGCTGCAGG
AATTCGGCACGAGGAGTANCAGGAGTGAAATCAAAGGATTTTATGATCCACCAAGAAGATTGCTGGG
CCAGCGACCAGGACCATATGATAGACCAATAGGAGGAAGAGGGGGTTATTATGGAGCTGGGCGTGGA
AGTATGTATGACAGAATGCGACGAGGAGGTGATGGATATGATGGTGGTATGTGTATCTAATGAACAA
AGGTTCTGTTGTCATTTTCTTAATGTTCCTGACACTTTGTCAAGAAATACAGAAATGGCAGTAATTTCA
GTACCTACTAGGTTTAATAAATCTGTTCATACAAATGTGGATTCCCATGGGCAGCTATGGGACTTGATT
GATGCATGTATTGGCACCTAGTTTACCTACACAAAAATTTTTAAGAGATTGCCATGTTTTGACCTTTTC
TGTTTGGCATCAGGATAAAAATTTTTATGTCTAAATGTGTGCTTTTCATACAAGTGTTAACTGCTTTTTT
AATATACACTAAAATGGGGTGTTGGAAAACTTGGTTGGAGGGGTTGCAAACTTGTAACAATATAAGTT
AACCATAGTGCTTGNCANAAGATACATTTTTATAAACTAGATAATAAAAGTTTATCTCTTGAAAGCTTAT
TCATGNATTTCTCCTTAAATTACACAGGTTATGGAGGTTTTGATGACTATGGNGGGTATAATAATTATT
GGCTTTTGGAAAANGATGGCTTTTGGATGACCCAAATGGAAAAATGGGAAAANGTTTGGGAAGGACC
TTGGTTTTTGGGGGGAACTTGGGGGAATCCAAAGTTTCNGGGTTTTTCATGGGGGGGGCANTTTTGNA
CCATAAGAAAAAGGACTGGNCNTTTTTCNGGCCACTGGNAAAAGGAAATTGGGNAAANTTTCTTTTTC
NCC
NACAGCTGGAGCTCCACCGCGGTGGCGGCCGCTCTAGAACTAGTGGATCCCCCGGGCTGCAGGAATTC
GGCACGAGGAGTAGCAGGAGTGAAATCAAAGGATTTTATGATCCACCAAGAAGATTGCTGGGCCAGC
GACCAGGACCATATGATAGACCAATAGGAGGAAGAGGGGGTTATTATGGAGCTGGGCGTGGAAGTAT
GTATGACAGAATGCGACGAGGAGGTGATGGATATGATGGTGGTATGTGTATCTAATGAACAAAGGTTC
TGTTGTCATTTTCTTAATGTTCCTGACACTTTGTCAAGAAATACAGAAATGGCAGTAATTTCAGTACCT
ACTAGGTTTAATAAATCTGTTCATACAAATGTGGATTCCCATGGGCAGCTATGGGACTTGATTGATGCA
TGTATTGGCACCTAGTTTACCTACACAAAAATTTTTAAGAGATTGCCATGTTTTGACCTTTTCTGTTTGG
CATCAGGATAAAAATTTTTATGTCTAAATGTGTGCTTTTCATACAAGTGTTAACTGCTTTTTTAATATAC
ACTAAAATGGGTGGTTGAAAACTTGGTTGGAGGGGTTGCAAACTTGTAACAATATAAGTTAACCATAG
TGCTTGTCAAAAGATCCATTTTTATAAACTAGATAATAAAAGTTTATTTNTTTTGAAAGCCTTATTCATG
GANTTTCTCCCTTAAAATAACNCAGGTTTATGNGGGNTTTGAAGACCTATGGGGGGCTATAAAAAATT
ATGGGTNTGGGAAANGAAGGGNTTTGATGACCNCAAATGGANAAAATGGGAAAAGGTTTGGGAAGG
ACCATGGGTTTTTGGGGGAACCTGGGGNAATCAAANTTCNGGTTTTTNAATGGGGGGCCATTTTGGNC
CATAAAGAAAAGGGACTGGCCNTTTTNGGGCCCCCTGGGAAAAAGGAAATTGGGTAAATTTNTTTNTC
CNCCCCTTAAA
TTNNNAAAACTGGAGCTCCCCGCGGTGGCGGCCGCTCTAGAACTAGTGGATCCCCCGGGCTGCAGGAA
TTCGGCACGAGGCCCCATTCCTGTGGCCCAGGGGCCCAGCTTCCAGTTCGGCAGTGTCAGAAGTGACT
TGTGGCTCTGCCTTGGTTGTGAGATGAGTACATTCCAAAGGAGCAGCGACGGGGAGGGGGCAGGCTCA
GCGATCCCTTGGAGGCTCAGAGCAGCTGGCCTGAGCTCCAGCTCGCGTCCAACGGAAGCTCGCGCCCC
TCAGAAACGACCCTGGCCAATGTCACCGCAGAGCCGAGGCGGTGTTGTCAGGGCTAATGTGAAATCTC
TTCCCGTGGCGCCGATCCTCCGGCTCTGCAGCGCCAGCTCGGACAGATTGAGGTCTCTGTGGCTTTGGT
GACCCTGCTTAAGCATTCCCACCGTGTCCCAGCTCTGCTCTCTGCCTGCCTCCCACTGCAGCCCCCCCC
TCACCTGGCTTGGGGAGGGGGGCAGTGCCCCAAGGCTAGGGGCTTTTGCTCAAGAAAAGATGTCAGG
CTTTCTGGGGTCGTCAGGCTTTGAGCTCTACCGGGGTCCAACTGACCTGGGATACCAGGTGTGCCCAG
AANATGCACCTCATNCCGTGCCTTNAGGAANGGCTAAGGAACAATTTTGGTAATTGGATTAAGTTTAC
CTCAGTTAACTCCCNTTGGGAAAACCTAACCCANTTTTTAACTTTCTGGCCTAAAAATGANTGTATTGN
NTANGGAAAANTGAANCTTTTAATTGCTTCCCAAAAAGGGGCCTTGGTTTTTCTTCTGGANTTAAAATTT
TTTTTTNTAAGGGGAANNANNNNNNNNNNNNNNNNCNCTCNNGGGNCCCCNGNNGGGGGGCNGGGG
GGNCCCAAAAAGGNNCCCCANGGGGCCCCCAAAAAGNGTTTTTTNNNGGGNGAAAAANGGGGAAA
NNCGGNCGNCGATTNAGCGTGNNNTGNNNTNTNNNNNCCNNNNCTCNCNCCCTCTTCTCCTNACCCGC
CACATGCACNTCCCCCCCCANGGGGNGNNAAGAGAATATGGNCCNNTNTNTTGGAGNNCAACTGGAG
CTNCACGCNNGAGGCAGGCCGCTNTAAAACTAGTNTATNCCANNNNTCCTGCTGNTATCTCAATCCNC
NTTGAATTTTTNTNCNNCTTCGCACANNTGANNTTCTTTCANNNNNGGNCCNNNTTNNGTNCNATAA
TTNTTNANTTCTCNNTTTGNNNTGNCTNGTNNTNGCTCGTGGACCATNGNAATCCTGNTNATNGTNNNT
AGAATTNNTCNTCTTNGGGGTNATATCTTNNAGCAGATCGTTTNCAANANANCTNANCNACGNGACNN
TNTCTATCNCNANANTNGNNTNNANTNNCTAGCCGGNCGANATNTNATCANGNTTNGCNNNNNANGN
TGNNNNANTNNTNNTNCNNNNNGTNTNAGCTTNTNTNNCGNTANNGCACTTTNACNTGNTCATTTTGT
ANAACTNNNNNNNNCNNNTCTNNTACGANNATCANCANTANNNCGANTNCNNTTNNNANNTNNCNNN
ANNCNCNNTCTAANNTNNNCTNNNNTNCGCTTTNNGATTNGGNCTAGNTNTCNNNNCANCAAANTTA
NGTNANGTCTNGCNNNANTAATTNTCCNANAGACACNTNTTNGATCTTTTTCGTNATGCTATNTGCGA
NNTANCTCTTNTANNACNNTCTNNTANNCGNNGATTTGCNGGCNGNNNNNACNGCAATCTTTNNNTN
NANNNTNNGTNANTNNNTACTGTNTNNTATCATNCCNNNCNATTNTNNATNANTGAGGTGNCGNTAN
GTAANTNGCGGGNTTTATCNTCGNNNTNNANNTNGTNNTGNACNGNNNNNCNGATTNTANGGTTTNTT
TGTNNNNNCNGNNANNCNNNNANTANTNNTCNANNNNNNTNTTCNNCATNGNACGNCGNTNNACCNT
ATAGNTAATTTTANNTNNNNTAGGNCGNGGGNNNNATAANANTTNNTAGTNANNCNTTNCAGTNNNT
ANCNGNCNANCGGNATGAAGTNNATCANATNCTNTNTAANNAGTGCAANCANNCNNNNGGANNNAG
CNNAGNTNTATNCTCNAGNNNANNGNANCANCNCNTCAGTNTAANNAGATGNANCAGCTGTGAGNTG
AGGCNGNGCGNTGNGTACAGAGNTNTNTGTATAGAANCNCANCNTGTATGNAGTCTGAGANNATCNN
ANATGNNGNNCNTGTNTCTCNNTATNNAGACTAGANTNTNNACTNTCTGAGANGACATCTATGNTNGA
NNNANANANANACNCGANANANATNCGAGATNNGATATGACGNTTNTNTNTNCCNACNNATGTATNT
ATNCNGNNTCTCANANGAGATGACNCACNACNCTNNCTCATACTTNTCTNTNCGTNCNNNTGTCTANN
NTATGTGAAGTATACACTAACGTCNTACATGTNANATGATNTACNAGCNNGNGGANNNANTGACNAG
TGTGACNAGACNACNCNTNACTAGNGTCGATAGNCNANATNNNGNNATANNNTCGTTNTCGCGTATN
AGCGCG
CCGCCNCCNCCTCNTNGANNTNNTTNNNGTNNTNGNNGTNNNNTANNNNNNNTNNTNNNNNNCCTCT
CATCNNCTNTGGNNGNCGGNNGCTCNGNNGNTNTNNNTNCNNNNCNGGGGGNGGTNNANGTGTNTNG
TTNNNNNNNNNTTNNAGCCCNATAGTGGAAGTCGTATNNCNAGNGGCAGTNCNCTCTNAANCTAGNG
GGATCCCCCGGACTGNCTNGANTNTNCCGCCNCAGGNNCTCTNNNTCCCTNCNCTCCTTANNTCTTTTG
TNCTNNNTGGNGCNNCNNCNNNCTCNTNTNTNTNNCCNCCTACNGCCNANNCNNCTCCCCCGNNTNTC
CCNNNCATNCCCCGCNCNNNNCCTCNNTTCTCTCCTCCNCCCNANCCNCCCTCNCCNNNNCTCTCGAC
NCNTNGTNGCNCTNGTGNTCAGANNCNTGTCCCNNTNCCCCNNCTCCAGNGCTCTTTCCNNNNTCNAGN
TCNCCCGNTCTCNNNTTCNAGTCCNCTNGACNNTTGTNNACNCCNCNCTNGTCCNNCTCCCNNTTAGA
CNCCNGCNCCCTNTCGCTCCNCNCNGNNTNTTCCNNNTACTTTCCCNCTCCNNNCACNCGNGTNTCCG
CCNTCTNNCCCCCTCCNTCTANAGNCNCNCCTGNTCCTNNATNCNNTNNCTNCNTATNCCTNNACTCN
NNNTNTCCCNCAGGNNCNTGTNCCCGTGNCGCCNCTNCCNCCTCANNTCTNTCNNCCCNTCACCNGCT
NCNTCCCNACNNGAGCCCTCCTNGTTNTGNNCCNCNAGNNCGGNTTCCCNNGACGNCNCTNTCCCNCA
TTTTNCNACCNGTNNCCTCGGNACNCCNCACTCCCCGCGCNNTNCCTCNACNNCCNCNNGNANCCACT
NCTATNCCTNNCGACTCCACNGCTTNCTGTCGNNTTGACCCCCCTNCNACGNCNTCCNTAGCNCCCCTC
TTGNNTTNCNGATCCNCNNNNNGTCCNGATGNCTTCCNGTTCCCCGTTNNGACTNCCGGAGNTTAGNC
ANTNTTTGTGNCCTACNNTTCCCTTGATCNCCCGCTNNNGCCNCNGGNGTCCCTCGANCGNCACCNTG
GANGNNCNGCNNTNTGNCGGCNNCCGNCTCAGACCGTNNCTGAANNTNTNCTCTNCCTTNANCGACN
ATCACGACCACCNGNNCNNTTCNCGNCACGCNTCGNCNTNACCTNNCNCCNNCTACCGTAGCTCCCNN
AGCTCCNCNTACCTNTGTTNATNNACCACANACGNTGCTNCGCAGTATCNCCCCNCGTAGTNCCNACG
CNNNTNTCCCAGCTCTGTNAGGGTCTCTGCGTATCAGCTCCCGCTGGTCGGNACTCCCCNATNTCTCTC
TCTNCTTNTNNCCGGCACTTAAANGAGNTACNAATNGATTACNNNATNTGACGTTACTANCTCGNNGC
ACGNGCNGCCCTATGCTCNGNNANNCCCTCTACNAATGGANGTANCCTNCNCNAAGGNATCTTTCGNA
TGGANCNACCGNACCNTNNGTACCGCGCCTTAGGNTGGCGCTCACNCTTGNTANNTNCGNNTCTTCGC
TCTTNNCNNNNTCNTCGCNCNNCNCC
GCCCTATANNGAGNNGTATTACAGNGGCGGCCGCTCTAGAACTAGTGGATCCCCCGGGGTGCAGGCGT
TCGACAGATAGCCGTAACTTCTGTGCTGTGCCAGCCGCATCCCTGAGACAAGATGGTGAAGGTCGGAG
TGAACGGATTCGGCCGCATCGGGCGCCTGGTCACCAGGGCTGCTTTTAATTCTGGCAAAGTGGACATC
GTCGCCATCAATGACCCCTTNATTGACCTTCACTACATGGTCTACATGTTCCAGTATGAATCCACCCAC
GGNAAGTTNAACGGCACAGTCAAGGCATANAACGGNAAGCTCGTNATNAATGGAAAGGCCANCANC
ATNTTCCAGGAGCGANANCCTGTCAACATCNANNGNGGTNATGNTTAGAGCTNNAACTNNTGGCGGA
ATNTTACTNGGGANTTAAACTACACATGTGANAANGNGTTNGGAGTAGTTNGAAAAGTGTGCCCNAA
ANAANGGTGNATNAATCTCAGCNNACTTTTTTGTTCGAAGCCCCCCATGNTTTTTGNATTGNGCTNTAN
AANTCACTNANAAAGTTTATACANACNTCCCTTNANAATNNGTCNCGNTAATGTTNTNNTGTGNNAAA
CTNAAACTGTNTTNTCCCCCCCCTCNNTCAANNNTNCATATCANNTANAAACTTTNTGCNTNTTGGGA
NGGGACATNATNNACACATTNGGGCATANCCATTNTACTTGTCACCCCANNAAACATANGTNGGTTNA
NNCCTCNTCNNGNGTAANNTCTNNACNTCTTTTNNTTTCCTNNNGNGGNTTNNNTCNNNAAAAATTAC
CCNCTCTNNTTCTTANTTATTGCGCTNNTCTNCNANACATNTNGNGTNATAGACNCNTTCTCTNANCN
TNNANNCCNCNNATNNTNATCNTNNNCTTTNNCNGTTNTGNNCGTNTCTCNCTATNCTCTANCATATTT
TATGNNGGTTGNNATNNTCTTNACCNTCANAANACTGNATNNAAAACCTCNCCCNACGNNCNTCNTTA
TAATACTNNCACTCNATTNGNNTNGNNNATTGTTNATGACANGG
AGCNCTATATGGAGNTGNATNNCAGTGGCGGCCGCTCTAGAACTAGTGGATCCCCCGGGCTGCAGGCC
CCGNTGGACCCAAGGCCCCCGTGGTGACAAGGGTGAGACAGGCGAACAGGGCGACAGAGGCATTAAG
GGTCACCGTGGCTTTTTTTGGTTTCNAGGGTCCCCCNGGCCTTCCNGGTTTTCTNGGGNANNANGGNCN
TTCCGAANCNTTTGNNCTTGTTGNNCCCNGGGNNCCCCTTGNTTTTGTTGNTTTTCCNGGNAAAAANGN
NNTAATNGNNTTCCAAGGCCCATTNGNNCCCCTNGGCCTTNAAGGNCCAATNGNGAANNTNGNCCTN
GTNGNNCTTCCNGGCCTTCTNGAACCCCTNGGNCCCCANAGTCCTTCCAACGGGGGGGTATACANATT
NAGANTTTTCTGTGCCCAAACACACTTNAAAAAAAAAGGTTCACNATGNNGGCGCTATATATACGNGC
TTTANATNANGCCATNTGTGGNCCGANACCGANCATCTAANGTGNNAACACCACTCTAAANANCTTGN
CNCNACATATAGAAAANTTCGNGACCNTTAAGGGCANCCAAAAAAACCGCGCCCNCATGTGGTGAAA
TCTAAAATATGCCNCTCTTTTTTGTGANAAGAGCNGAAAAATTGTGTTTTTTGCCCACAAANAGGTGGT
GNACNTGGNATGACAATTAANAGNGGTTTTTGGNACAATNGTGNAAAACCCGGAAGAAACNNTTTGT
NTAANNCCNNCTNTTCACCCACCAAGTNGNGCCNCNAAAAAAAAATGTTGTTTTTATTCNANNAACAC
CCCCCGAAANAAAAAGGACNCTNNTTTTNGCTNCCGAAAAAAAATTAATCANCCGGAATTTTTACTNN
TTTAATAATTTTTGGNCCANAAGNTCCAANCCTTTCNNGANNGATGGGGGNATCGAAACAAGGTTTNT
TTTTCTTGCCGCANGGNACAACCAAGGNNNTCTCCAAAAAAAATTTNTTCTACTTNT
AGCNCTATATGGAGNTGNATNNCAGTGGCGGCCGCTCTAGAACTAGTGGATCCCCCGGGCTGCAGGCC
CCGNTGGACCCAAGGCCCCCGTGGTGACAAGGGTGAGACAGGCGAACAGGGCGACAGAGGCATTAAG
GGTCACCGTGGCTTTTTTTGGTTTCNAGGGTCCCCCNGGCCTTCCNGGTTTTCTNGGGNANNANGGNCN
TTCCGAANCNTTTGNNCTTGTTGNNCCCNGGGNNCCCCTTGNTTTTGTTGNTTTTCCNGGNAAAAANGN
NNTAATNGNNTTCCAAGGCCCATTNGNNCCCCTNGGCCTTNAAGGNCCAATNGNGAANNTNGNCCTN
GTNGNNCTTCCNGGCCTTCTNGAACCCCTNGGNCCCCANAGTCCTTCCAACGGGGGGGTATACANATT
NAGANTTTTCTGTGCCCAAACACACTTNAAAAAAAAAGGTTCACNATGNNGGGGCTATATATACGNGC
TTTANATNANGCCATNTGTGGNCCGANACCGANCATCTAANGTGNNAACACCACTCTAAANANCTTGN
CNCNACATATAGAAAANTTCGNGACCNTTAAGGGCANCCAAAAAAACCGCGCCGNCATGTGGTGAAA
TCTAAAATATGCCNCTCTTTTTTGTGANAAGAGCNGAAAAATTGTGTTTTTTGCCCACAAANAGGTGGT
GNACNTGGNATGACAATTAANAGNGGTTTTTGGNACAATNGTGNAAAACCCGGAAGAAACNNTTTGT
NTAANNCCNNCTNTTCACCCACCAAGTNGNGCCNCNAAAAAAAAATGTTGTTTTTATTCNANNAACAC
CCCCCGAAANAAAAAGGACNCTNNTTTTNGCTNCCGAAAAAAAATTAATCANCCGGAATTTTTACTNN
TTTAATAATTTTTGGNCCANAAGNTCCAANCCTTTCNNGANNGATGGGGGNATCCAAACAAGGTTTNT
TTTTCTTGCCGCANGGNACAACCAAGGNNNTCTCCAAAAAAAATTTNTTCTACTTNT
TTTANAAAANCTGGAGCTCCACCGCGGTGGCGGCGGCTCTAGAACTAGTGGATCCCCCGGGCTGCAGG
AATTCGGCACGAGGCNNTNCCCATCNNNNANNNCTGNNNGGANTNATGGGGACGNTTGAAANACANA
CTCACTACATNCTNCCCNTANACCNGCCNAGGTGGNNCANGTGACGATGTGATCANAGGGTGGANCC
TGNNAATCACGAANANTNCCANAACTNNNGGACCTATGCCTTAAGGAANGGGTAGNCTTAGCTCTGA
TANTCCAGGTANTNAATTGTNNCATTGGAAATNNANCATNGANGAGCCTGGCNCTTNCTATGATCATG
GNTCCATGGNATTTAANCNTTTAAACNCGANAACNNCNCNCTTNCCAANTTTTAANACTNNAATTCCA
TCCTTTNTTNATAGNNNNCAATNNTNNCTTTTGTTGNNCTTNTNTNTAAAAAAAACAATANNTNTNNN
NTTNGNTTTTNCANATCCCAAANNNCCCNTTTTTTTTNNTNGNAANTGTTTNTNTNNCTNTNNCAANCC
TTATTCTTNCTTTNTNTGTTGTAATNNCCCNNNNNNTNTNTNNTCNTNNTNTNNTTTTTCTTCCANTCC
NTCNNNTNCGTTTANNTCNNTNNCTCNTATNTTTTNTTTCTACTNCNNNCNNTTTAATNTNTGTGNTTN
NTTTCTTTCCTTATGTACCTNTANCNTTTTTTTNTTATTNTCTATGTATNTNNTNNTTTATTTCTCNCNT
TNANTATAAATCNANNCTNNNNTTNCCACTNTNNNNTCNNTNCGATNTNACCNTTTTTTTNNTCCTCCC
GTCGTTTNNCNATNNTAANNNNTTTNCTCTCNTTNTCAAGTCATNTTCCNCCTTTCTTNTNTCNNNTNTT
CNTTNNNTATCAAANTTTNANTATANNCNTNTCNNTTTTTCTNTCTNTATNTNANCTTNTTTTTTNNTCN
AATATTNCTACCANTNATTTNATANTTTTNTTTNTNCTTTCTNCANTTTGNTTATCCNAGCTNNTNTCTC
TTCNNTGCTNNNTNNTCNNGTTCNNNTCNNTTATANCAGTTTNCATNTNNTNCCTNCT
TTNNACAAANCTGGAGCTCCCCGCGGTGGCGGCCGCTCTAGAACTAGTGGATCCCCCGGGCTGCAGGG
CTTGGGCTGCGTGGGGATTTTGTAATGCCGACCGGAGACTTTGATTCGAAGCCCAGTTGGGCGGACCA
GGTGGAAGAGGAAGGAGAGGACGACAAATGTGTCACCAGCGAGCTCCTCAAGGGGATCCCCCTGGCC
ACTGGGGATACCAGTCCAGAGCCTGAGCTACTGCCGGGAGCTCCACTACCGCGTCCCAAGGAGGTCAT
CAATGGAAACATCAAGACAGTGACGGAGTATAAGATAGATGAGGATGGCAAGAAGTTCAAGATTGTC
CGCACCTTCAGAATTGAGACCCGGAAGGCCTCAAAGGCTGTGGCAAGGAGGAAGAACTGGAAGAAGT
TTGGGAACTCAGAATTTGACCCACCGGGGCCCAACGTAGCTACCACCACAGTCAGCGATGATGTATCC
ATGACATTCATCACCAGCAAAGAGGATCTGAACTGCCAGGAAGAGGAGGATCCAATGAACAAGCTCA
AGGGCCAGAAGATAGTGTCCTTGCCGAATCTTGCAAGGGCGACCACTGGACCACCCGCTTGCCCCTAC
AAGGACACGCTGGGGCCCATGCANAAGGAGCTGGGCCGAACAGCTTGGGCCTGTCCCTGGCGAAAAA
GGAAAANCTCCCCGGANAGCTGGAACCTGGGCNNGNCCCTTAAAACAAGAATTGGGAAGTACGTTGC
CTTCGANCCTTGGCCAAAGGGGCCAGCCCCCGNGGGGGAGTCCNTGCACCCAANCCGANAACTGATG
ACAATGCCCCCTTTCCNGTTANCCAACCTTTTTTGAGGACCCTTCNGGNAACCCACCTGGAGGAACTTT
TTCCGGNCCTTTNGGTTCCNTTTTCCCGATTTTANCCTGGGAAAAGGNAAAAAACCCCCGGCCN
TNNNNNAAAAGCTGGAGCTCCACCGCGGTGGCGGCCGCTCTAGAACTAGTGGATCCCCCGGGGTGCA
GGGCCTAAGTTCGACCCCAACGAGATAAAAGTCGTGTACCTGAGGTGCACCGGTGGGGAAGTCGGTG
CCACGTCTGCCCTGGCCCCCAAGATCGGCCCTCTGGGTCTGTCTCCAAAAAAGGTCGGTGATGACATC
GCCAAGGCAACTGGTGATTGGAAGGGTCTGAGGATTACAGTGAAACTGACCATTCAGAACAGACAAG
CCCAGATTGAGGTGGTACCTTCTGCTTCTGCCCTGATCATCAAAGCCCTCAAGGAACCACCAAGGGAC
AGAAAGAAGCAGAAAAACATTAAGCACAGTGGAAACATTACTTTTGATGAGATCGTCAACATTGCCC
GGCAGATGCGGCATCGGTCTCTAGCTAGAGAACTTTCTGGAACCATTAAAGAGATCCTGGGGACCGCC
CAGTCTGTGGGCTGCAATGTTGATGGCCGCCACCCTCATGACATCATTGATGATATCAACAGTGGCGC
AGTGGAGTGCCCCGCTAGTTAAGAACTGCAAAGGAAAATAAAGGACCATTTGACAACCAAAAAAAAA
AAAAAAAAAAAACTTGGGGGGGGGCCCGGGNCCCAATTTNCCCTTATGGGGGGGGGNNNNNTNAAA
AANNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNN
TNNNACAAAGCTGGAGCTCCCCGCGGTGGCGGCCGCTCTAGAACTAGTGGATCCCCCGGGCTGCAGG
AAAAATTGTAAACAATGCATTCATCGACCTTCCAGCCCCATCAAACATTTCATCATGATGAAATTTCGG
TTCCCTCCTGGGAATCTGCCTAATCCTACAAATCCTCACAGGCCTATTCCTAGCAATACACTAGACATC
CGACACAACAACAGCATTCTCCTCTGTTACCCATATCTGCCGAGACGTGAACTACGGCTGAATCATCC
GATACATACACGCAAACGGAGCTTCAATGTTTTTTATCTGCTTATATATGCACGTAGGACGAGGCTTAT
ATTACGGGTCTTACACTTTTCTAGAAACATGAAATATTGGAGTAATCCTTCTGCTCACAGTAATAGCCA
CAGCATTTATAGGATACGTCCTACCATGAGGACAAATATGATTTCTGAGGAGCAACAGTCATCACCAAG
GTCTTATCAGCAATCCCATACATCGGCACAAATTTAGTCGAATGAATCTGAGGCGGATTCTCAGTAGA
CAAAGCAACCCTTACCCGATTCTTCGCTTTCCATTTTATCCTTCCATTTATCATCATAGCAATTGCCATA
AGTCCACCTACTATTCCTNCACGAAACAGGCTTCAACAACCCAACAGGAATTTTCCTCAGACGTAGACA
AAATCCCATTCCACCCCTACTATACCATTAAGGGACATCTTANGGGNCCCTCTTACTAATTCTAGCTCT
AATACTACTAATACTATTCGGACCCGGACTTCTTNGGAGAACCCANATAACTTCCNCCCCAGCCNATT
CAANTTAAAACACCCCCTTAAAATTAAAACCCGGGGGGAACCTTNTTANTTGCATTCCNCAAATNTTA
CGAAACAATNCCCCAAAAAA
NNNACTGGAGCTCCACCGCGGGGGCGGCCGCTCTAGAACTAGTGGATCCCCCGGGCTGCAGGAATTC
GGCACGAGGAAAAGATCCAAATAAAGAAAAACAAGCATTTGTGAAAACAGTTAAAGATCAAGCTTCT
GAAATTCAGGTGACAATGATGCTCACCGAAAGTTGTAAGCTCCGAGGTCTTCATCACAGAAATCTTCT
TCCTATTACCAATGTGTGTATAGAAGAAGGAGAAAAGCCCATGGTGATATTGCCTTACATGAATTGGG
GGAATCTTAAATTGTTTTTACGGCAGTGCAAATTAGTAGAGGCCAATAATCCGCAGGCAATTTCTCAA
CAAGACCTGGTCCACATGGCTATTCAGATTGCCTGTGGAATGAGCTATCTGGCCAGAAGGGAAGTCAT
CCACAAAGACCTGGCTGCTAGGAACTGTGTCATTGATGACACACTTCAAGTTAAGATCACAGACAATG
CCCTCTCTAGAGACTTGTTCCCCATGGACTATCATTGTTTGGGAGACAATGAAAACAGGCCGGTTAGG
TGGATGGCTCTTGAAAGTCTGGTTAATAATGAGTTCTCCAGCGCTAGTGATGTGTGGGCCTTTGGAGTG
ACGCTGTGGGAACTCATGACTCTGGGCCAGACACCCTACGTGGACATCGACCCGTTTGAGATGGCCGC
ATACTTGAAAAGATGGTTACCGGATAGCCCAACCAATCAACTGTCCTGATGAACTATTTGCCCGTGAT
GGCTTTGTTGCTGGGCCTTAAAATCCCGGAGGAGAAGGCCCAAGTTTCAGCAGCTTGGGTCCGAGTGGC
TNACANNAATTCCCTGGCAGCCCCTGGGGGGCCCATTGTCTNGACTTTTGCTGTCACAAGTCCTCCNAA
GCGTNCAGGAAGGAAAGGTGCCCTGCCCGGCCCTNCTTGGGANCCCAATTTTNGGATGCCCCAATACC
TCACNGCAANGNCCCCAGAANNNATTTTGTTTTCCAAAAACCNGGGNCCTAGGNA
NAAAACTGGAGCTCCACCGCGGGTGGCGGCCGCTCTAGAACTAGTGGATCCCCCGGGCTGCAGGCTGC
ACGTCTAGGCTCGCCCGCGCCGGCTCCGACCAAATCCGATGGGACTCCGTGATGCTTCTGTCCTTGGCC
TTTAACGAAAACTGTCCAAAAAAGGGAAGAAGTGTTTGGTGGGTGGGGGGAGGAGGGGGGNCCCCCTT
TTCCGTGGGCACGGGGAGGAAGGCCGGCCCGTTATGGNCCNNCCAACCCCGGGTCTGTCCCANCCCTT
AACTTGAGCNTGGCCCNCGTCCCTTCTAAAGGCNCCNGGGCCAAACNCNTNTTGGGACTGAGGGCTTN
CTTTAAGGAAAACCCTTGCTGGTTTANGCCCTGNANGGNATTTGATCATTATATTTTANCATTTAATTC
TCTCCTATTTATTGACTTTTGNCAATTACTNANGNTTGNGAAAAAAAAAAAAAAAAAAAAAANNNNG
GGGGGGGGCCCNGNNCCNATTTNNCCTTANNNGNNNNNNNATNANANNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNC
NCTCACTNAGCNCTNANGTCTAGTACNCNANCNGCCCANCATTANTTTNAAGCCCNATANTGNAGTTC
GNCATTACNNGGNCATNCTCCGCCTCCTATNACATAGNNTCTNANNACCCANTACCTNTATNATANTA
NTTACCNTACATCCNAANTTTATTTNNTCNATGCTTACTAAACCNNANAACNTTNANANCNNNNCTTN
TTCATATNATAAAACNNATNTTCCNCNTNNNTTNACTCTACNNANCTATTTACNCTNATNATTCNNATT
NTCTGNCCNTTCNNNCTCCNCCATTNCTACNTTNAATAANNNTNNTATNANTATNCNNTCAATCGTCA
CCANATAANNNTTNCATTTTTNNTTTNAACNANNNTCTNTCAANCACCCTTTATNATNCTCTTATTANN
NNCANTNNNCNNCCCCNNNANTNTTTNTTTTNNNANATCANTNANNCGANAATTTACNCTTTCTTCAA
TTATNNTAAANNCTCCCTANCANCCTCNTCTTCTCTANNAATNTNTCNCNACNGCCCNCCANTTCGAA
CAATNCTCNNANTNTCATCNACCNCCCNTTAACNTATNCTCNCANNTNTNTCNNTNCCNNNACANTGN
CNACATTTTATNTATATCNNNTTNTNNNNAACNGACNNTNNANCNTNNAATTCATGCAANNNANATNT
TTNNANGTTTACCTTAANTTATCANCTNTTCCTATAATCTNNTANCTNNNTNCTNTATNNNANCNNATC
TAAACAATANNCTGCCTTTTCCTCNATCTANCATTGTTATATANANTNNTCCTATNTNNNNNCAATTNC
CTAANCCCNNNCTGANTCAACCTAACANTCCAAACNCCATNNNNNNNTTANCTNTACNTTTGCCTNCN
ATAANCTTCANACTTCNCTCNCNNCANNTCTCTTNNNTNCNCTACAAACTTTANCANANNTCNTCTTTN
NTTNATATAANAAATNNCAATTANNATNANNNAAGCNNNNTAACANTTNNTGTGNCTTCTCNNTCTAC
TTACCCNCCACNATCCCTCACCCNTCGTNNCACCTCTNATNANTATATNANTTNTTCNAACCANAGCAC
NGCGNCTCANCTTCTNTNCANTATNATTATCTCATCTNTTCTNCTCCTCCNCTCNTCNATCAACGCTTTN
CNTANNNTAANCAAANCTATCNNTCATACNCTTTTNATTTATCACCTNNCTTATATTNNTCNNANNCNA
NATATANCACCACTNTGCTCAACNANTCNTCTTCTNATNNTNCNACANCNCANATANNCCNCTGNTTA
CNCTTCCNNNTAANNNNCGCATNTATATACATCTNCNCTCNNANACANTNCNCTNCACNANNNATAAT
NNATACNTCTNTACTCTATATNNTCCACNCNNTGTNNTCACCTACACANACANANTCTTCTNNTNATAT
ATCNCGNTACACTTATANATNANTNCNNGCG
TTNAGCCCTTATANTGAGTNGNANTTACAAGGGCGGCCGCTCTAGAACTAGTGGATCCCCCGGGCTGC
AGGCTGCACGTCTAGGCTCGCCCGCGCCGGCTCCGACCAAATCCGATGGGACTCCGTGATGCTTCTGT
CCTTGGCCTTTAACGNAAAACNGTCCAAAAANGGNAAAAGNGTTGNNGGNGGGGAAGNAGGGGGNC
CCCCTTTTCNGTGGNCNCGGGNNGNAGGCCCGTTCTTTATTNTCCTNCCNATNCCNCTTNTNNCCCATN
CCNNTAATTTNTCTNGTCNGTCGTCNATTCCNTAANNCCTCTNTNNTCAAAAANCTTTNTNNGGANTAN
TGCTTTCTCTTTNNGGANNAANCTTCTTTTTTTTACCNCTTGNNCNGGGTTTTNCTCCNTTTTATTNTCN
TCCNTTANNCTCCTNTNCTATTNTTNTCANATTNNTACAGTTCNCTNTNTTTTCTCNTANNNATAANNN
AANANTNNTTCTTTCTNNNNGGNNCNNNTCTCCNCCTCNTNTNNTTTTTATTTTNTGCCNNACTNTNCT
NAATCCCTTNTNTNCTATNTCTCTNCCCNTNNCTNTGCCTTTNTNNATTNCNNNNTCNTTNCNCNNTCT
CNTTCNNTCNTNNCNTANNNCTTTNCNATCTCTTNTNCCCCTTCNTNNTTNNTNNNTNTTTNTTCATTA
TNNCTTTTTCTTNNNTTTTANNNANTNANTATNNTNTTTTNCTTCCTNTNTCCNTANNTNATTNCNCNN
NTTCTTTNTTAGTNNNTCNNNNTCTCTNCTTANNTTTNTTNNNCTCTTCNTTTNNNNCTCTNANNTTCNC
NTTNTTCNNNCTNNNNCTTNNTTNNNNTNCNCTTNTTATNNTTNNNTTATNTTTNTNTCCTTTNNNTTNT
NANTTTNANTTNNTTANNNNNNNAAATTATCNTTTACAGNNNTNTTTANTCTACTTTCTCNNTCTCNAT
TCNNCNNNNAATNTNCNNTTNTNCCNCANTNTCNNNTACNTNTTCTNTCCTNNTCTNTNNTANCG
TTNAGCCCTTATANTGAGTNGNANTTACAAGGGCGGCCGCTCTAGAACTAGTGGATCCCCCGGGCTGC
AGGCTGCACGTCTAGGCTCGCCCGCGCCGGCTCCGACCAAATCCGATGGGACTCCGTGATGCTTCTGT
CCTTGGCCTTTAACGNAAAACNGTCCAAAAANGGNAAAAGNGTTGNNGGNGGGGAAGNAGGGGGNC
CCCCTTTTCNGTGGNCNCGGGNNGNAGGCCCGTTCTTTATNTCCTNCCNATNCCNCTTNTNNCCCATN
CCNNTAATTNTCTNGTCNGTCGTCNATTCCNTAANNCCTCTNTNNTCAAAAANCTTTNTNNGGANTAN
TGCTTTCTCTTTNNGGANNAANCTTCTTTTTTTTACCNCTTGNNCNGGGTTTTNCTCCNTTTTATTNTCN
TCCNTTANNCTCCTNTNCTATTNTTNTCANATTNNTACAGTNCNCTNTNTTTTCTCNTANNNATAANNN
AANANTNNTTCTTTCTNNNNGGNNCNNNTCTCCNCCTCNTNTNNTTTTTATTTTNTGCCNNACTNTNCT
NAATCCCTTNTNTNCTATNTCTCTNCCCNTNNCTNTCCCTTTNTNNATTNCNNNNTCNTTNCNCNNTCT
CNTTCNNTCNTNNCNTANNNCTTTNCNATCTCTTNTNCCCCTTCNTNNTTNNTNNNTNTTTTNTTCATTA
TNNCTTTTTCTTNNNTTTTANNNANTNANTATNNTNTTTTNCTTCCTNTNTCCNTANNTNATTNCNCNN
NTTCTTNTTAGTNNNTCNNNNTCTCTNCTTANNTTTNTTNNNCTCTTCNTTTNNNNCTCTNANNTTCNC
NTTNTTCNNNCTNNNNCTTNNTTNNNNTNCNTTNTTATNNTTNNNTTATNTTTNTNTCCTTTNNNTTNT
NANTTTNANTTNNTTANNNNNNNAAATTATCNTTTACAGNNNTNTTTANTCTACTTTCTCNNTCTCNAT
TGNNCNNNNAATNTNCNNTTNTNCCNCANTNTCNNNTACNTNTTCTNTCCTNNTCTNTNNTANCG
NNAAAAGCTGGAGCTCCACCGCGGGGGCGGCCGCTCTAGAACTAGTGGATCCCCCGGGCTGCAGGAA
TTCGGCACGAGGGTTGGTTAGAGATGCTCTTGAAAGACAAGAGAGATAAGAGGTGTAGAGCCAAGTC
TTCTAGGTGGCTTGCCCAGGTCACTCTACCTCTGGCCTCTGATTCTCAACATACATACCTACATGGCTG
CTCTTGTTAGTACAGNGTTGATTCTCTGGAAATTANCCGCATGCCTGCAGCTTGCTTCGTGTGAGGATA
ATGTCACCACCAGAATGGCTGCTCTGACCATTTGCCTGGGGGCTTTCTCATGGCTTTTTGAACAAGGAN
GCTGGGCANCCTAAAAATANTTCCACCNTTACNGCATNGGTTATGCCTTGGNGGGAAGGGGCCTGGNA
TACTTTTCACNTTACAATTCTTAAGAGAAAGANTAGGGCCTTACCTAATCCAAGNCATTAAAATTAAA
AAATGGNCTTNAGNNCNTGGANGTTTGGACCAAAATTTNTTTTGAATTTAACCCCTGGCCCAAGTAAA
TGGTTGGACNCCTNAGCCAGTTNTTAATNCNGGNCCAAAAACCTGAANGGANCGAATNATTANCTGC
NNGGTACCCATTGACNTTCTAACTTCCTNTTGGCCCCTTATNGCAATNCCNTTAGGAATGAAAAACCT
TTTGGGATTGGNGAACCCAACCTGAAANGGGANTTCCAAGNCCTTTGGTTGAAACCAAANACAANTTC
CTNTTGGCCCCANGGGAAACCTTAAAATTAAAATNTNNCCTGGAAACCCGGCCTNGGGGCCATNCCCA
AAAAAACTGGGCCCTTTAGGCCATTTNTTTTAATTCCCATAAAATCNGGNCCTTTGGGGNGGAAACCT
GGACCCAAATGGGTTGGGGTNGGGTTTTTTTTNCCTTGGNNTTNGGGAANGGGGTTTTNCCCAAAAGG
TTTTNCCCCCCTTTCNTTTTTGGGAANGGGGAAATTGT
TTNAACACAGCTGGAGCTCCACCGCGGTGGCGGCCGCTCTAGAACTAGTGGATCCCCCGGGGTGCAGG
AATTCGGCACGAGGATATCNCACTGGTAAAGAGATGCAGTTGAAGAAACCTGCTGAGTTGAAGATTT
CCTAAATTTACTAAAGGGAATAATCTGGGTTTCTTAACTGCAGGGGTTTTGAAGATATGACATCCTA
GAGTAATATCAGAGGATACTTGGAATTACCAGGGGCTAATTTAGAAAAGAAATTGTTCTTTTAAAAGG
AAAGAAATTTCAGTAAGAATAATCAGATTGGCCCAAGGAGCAGGTGTTCAGAGTGTTACTGAGTTAATA
TTTACTACTAAAATACTTAAAGTAAAATAAGTAATTTTAGGAAATGTCTTGATTGAACAAAGTTATTTC
TTAGAACAGTTTATGTTCTATCAACGATGTTTAAGTATTTCTATCAATTATGGAAATCTCTAGTTGAAA
GAATTCTGGAATTCATGATAAGGAATCAAGATTTGAAACTTTGAAAATTATACTGAATAGTTGCTAAT
CTATAAAGATGCTTTTTAACATTGTACATTTAGGAAACATGGGATTGGCCTTATTTTATACATACTCTTT
TAAAATATTGNCATTGCTTAAGACTTTTCTTGAGAGTGAATTACCTGGGAATAACATAAATTCCATGCA
ATTTTAAGTATTTGCTATTNAAAACCAGGTACCTTTAATTGNGGGATATGAATCTGACTAAATGNGCTN
TTACCAGGNAATACTATGNCATTCTACCTTATCNTTTTGGAAAGGTATTGCTTGAATGTCCTTAAAGGT
TTCTACCNCATAGNGATTCTGGAAGTTTTTCCAGTTTGNNTTTCCCNCTGGAAAAATTGGGAGTGGAAA
TTTTTCCCAAAACTTTTACNTTTCAATTAACTTTAGGAAGA
TTGAANACAAGCTGGAGCTCCACCGCGGGGGCGGCCGCTCTAGAACTAGTGGATCCCCCGGGCTGCA
GGNNCNAGNTNTTATGNTTTTTNNTACCGNNAACCTCANNNNNTNNNCAGGGGCNGATTNAAANCTA
TNNTGNGTCGNANNANATANNNGNTTTNTNTNNNANAANGNGGGNCNNAANTNGNGGGNCNNAGGG
NAAAAACCNNCCCNTTNNNCTTCNNNANNGNNGGAAAGGNCNTTACNCNCCCNTTTTTTTTTTAAAAA
NGGNNCNCCNNTTNACCCCCCNNNNNAGNANNNNTTNCCCGGGAAAACCGGGNANNNNTTNAAGGG
CNTNCCNNAAAAANCCNNTNCCTTTNNNAAAATTCCNNTTTAAATNAAAGNNCCNNANTTNNCGGGG
NANNTTAAAANNAGNTTTTNNCNNCCGNTTTNNTANCCNNNAANAAAACCCNTTAAAANCNGNNTTG
GGGGGGAAATTTTTNNNTTTTTTTTGGGAANTANNACCNNNNAAANNTTTGNNAAANGNTNNGNTTGA
AAAANANTTTTCCCCCTNAAATNTTTTAAATTCCAAAANTNNTNTTTTNNGGGAAAAATTTTNNGGGA
AATTTTTCCNNACCGTTTTNGGGGGGGNNAATTTTTNCCAATTAGGGGNNCCCCCCTNGNNTGGAATT
TCTGGGAAGNGGGGNAAATTTTCACTTTTTCCCCAAANGANAAAAANGGNTTAAAATTTTTTTTCCCC
CCNCAGAAACNTTTTNNNNCCCTTTTTNCCGNNGGGGGGGGGGNAAATTCGGGGGCNCTTTTTTAANG
GNCCCCCCCTCCNGTTTCCCAAAAAAANCCCCNNTGGGGGGGGCCCCANNNGGTTTTTTTCNCCTTGG
GGGGGGAANTTNTNTTTTNAAAAAAGGGTNTTTTTTTTTTNCCGGGCCGNGGNNGGGGNTTTCCCCNG
GGNTTTTTTTTTTTTNACCAAAAGGGGGGNGTTTTGGTTNCCNNT
TNNNNAAAACTGGACTCCACCGCGGTGGCGGCCGCTCTAGAACTAGTGGATCCCCCGGGCTGCAGGA
GCTGGANGGNTGCATGCATTGCCGCGTGTGCGCCAAGCAGCTGAGCGAGTTGTGTACCGAGCGCGACC
CCTGCGATCCGCACAAGGGCCTCTTCTGCGACTTCTGGCTCCCCGGNTCAACCGCAAGATNGGTAGTG
TGCACCAGGTAAAGATGGTGCCCCCTGCGTTTTCGGAGGAAGTGTGTACCAGAGCGGAGAGTCTTTCC
AGAGCAGCTGCAAATACGAGTGCACGTGTCTGGACGGGTCGGTGGGCTGCGTGCCCCTATGCAGCGTG
GACGTCCGCCTGCCCAGCCCCGACTGCCCCTTCCCTCGGAGGGTCAAACTGCCCGGGAAATGCTGCGA
GGAATGGGTGTGTGATGAGCCCAAGGANCACACCGTGGTCGGCCCTGCGCTCGCAGCTTACCGGCCGG
AAGACACGTTTGNCCCAGACCCAACCATGATGCGAGCCAACTGCCTGGTCCANACCACAGAGTGGAGT
GCCTGTTCCAAGACCTGNNGAATGGGCATTTCCACCCGGGTTACCAATGACAACGCATTCTGCAGGCT
GGAGAAACAANAGCCGCCTCTGCATGGTCAGGCCTTGCGAACTGAGCCTGGAGGAGAACATTAAGAAA
GGCAAAAAGTGCATTCCGGACCCCCAAATCTNCAAGCCTATCAAGTTTGAGCTTTTNTGGTTGCNCC
AACCNTGAAAAACNTACCGGGCTTAAATTTTGGGGAATGNGCCCAANACGGGCCGGNGGTTGCACCC
CCCCAAANAACCCCCACCCTTTCCCGNNGGAGNTTAAAGGGNNCCCGGANGGGGGAANGGNTNTTNA
ATNAAAAACCTGNAATTTTTAATNAAAAAAACTTGNGCCCCTNNN
GCCTATNTGGAGCTNCATCNCNGTGGCGGCCGCTCTAGAACTAGTGGATCCCCCGGGCTGCAGGCTTG
CTACCTTCTGCCACCGTTCCATGGTCTCTGGAATCCTTCCCACCAATGAAAGCCTTCGGTCCTGGCAT
TGGCCTTCCACCTAGACCGGCTTTGCCCTCTCTCTGCCATCCTCGGTCCTCCAGAGACCACACTGCCTG
TCACAAAGAAAACCACCACCTCGGGCCTGCCAGCCATCAACTTGGAAAATCGGGCCAGCTGTCTGCTG
CTCACGTCTCCCAGGTCCAGACCAAACGGGATCAACAACCCACAGTGGTGACTCAGTCTCTTACCCCA
GGAGCCAAAGCAACACAAAACAATTGGTCAAACATCGTTGGGTCCGATTCTGAGAGTAGGTGATTTCT
AGACATGTCTTTCAGGTGACCCTCAACTACCTCAGGTTTGAAGGCCCTAAATTAAGCTGATACTACTCA
TGGGGACAGACCGAGGAGGGTTCGGAGAAATCTTGGTGATAACGCACTTGTCTCAAACTTACAACAA
ACTCACGTTTCGGTTTGCAGGGGGTGGGGTTGGGGTTTGTTTTTCTTCTGTTTTTAACCTTTTCCCCCCA
AATTTTCCATTAAGAATAGTTCCTACAAATGAAATTTCATTTCTCACTGAGTGCCTACCTTCCCAGGGG
CAGGTANNCCCTGGGNTTTGTTTCCTTCCAAGCAGTACTTTTATTATTNGGNTATTAANAACTTTTCTTT
GTATAATACCATTGGNATCTGTGCTTTTCAATTTTTTCAANTAAANACCTTCNAACCCTGGCAAAANAA
AAAAANAAAAATNCNGGNGGGGGGGCCCCGGTACCCANNANGNGNCTNNNANNGTGACNTTTTTTCN
NNGCNGTGTTATANANNTNTTNAGTGTCTTTTTNTTCTCNATTCGNTTTCATTCATGCGTTANGCTCN
NNTNNNNTNGCNNTTANTCTCNGNGNNNCTCNTGCNNTNTNNCTTCGNCCC
ACNCNNTNTCANNNTANTAATTTTCTNTTTNNTTTTNTTTNTTTTTNTNNNTNNTACTNANANNNNTTCT
TTNCANTCCANGATNTCGTNTNNNTACTCCTNNGNANTNTNNNANGTTTGTCTCNTAATATNTTTNAGN
ANAACTGGAGCTCCANCGCGGNGGGCGGCCGCTCTAGAAACTAGTGGATCCCCCGGNCTGCAGGCTG
CNNTGTTTTATNNTNTNNTTNNACCGTATANCATCNNNTCCAANTTGNNNTTNNNGTCTTCNTTTATNC
TCTTCTCNNTTNAATTAGACNNTTTTTTTNAANTTNATNTTNTTCTTTTNNCATTTNTGTCNTNTATTNTT
TTTNGTATANNTGNTNCNAGTNTTTNTTTTNATNANATTNTCTCTTANTTTTTTNCTNCNNNCTCCTANT
TTTATACNTTTNTNTTGATTNCTANNTTANAAANNTTCGTTTTTGGATNTNTATTCTNTTTTTNTTAATG
NTTANTNNCTTCNNTTCNTTACTTCANNTNTTTATNGTNTAANATTANGACTTATATATNTACTANTTT
NTTNANTNGTATTCGTNNCTTNNTCNNTTATTTNNTCNNCTTGATNATNNTNTGNNTGNCAGTNTTTT
CNNTTTNNATNTTATNTTTTNNNTTCTTTAANNTNNNTTTTTTNNTATNNNACNTTNGNANTNTATTATT
TTNATNNNTTNTTNCNTCNTANTNTATNNAGANNTTCCATANTTNTNTCCTTTNAANNTNTTTCNNTCT
TTCTAATTCTNTCNNNNAGANTCAANCNATNTTNTTTNTCTTNTNTTTNNTANTNCNTCCNTNTTTCTCT
NGAAAAATCCATNATCGTTNNAANTGAAAAANTATNTNTCGTTTNTTCATTNCNCTATNCNTTCNTACT
ACANNNATCTCATTGTTCATTANTTTCCATTNCGATANCGNGNGNNNTGNTTTCNTNTNTATCANNTNN
CNACTNTNTNGCTAATNTCNANNNNACCGANTNGGGNTCCNTTTNNNNATCNNNTTTTCTCTACAGAN
GGGCTTCCCCGTTTTAACNCCCCCCCCNCCCACANAATNTNNGAAAAANCTCTCCCTTCCTCCCCCTTN
TCTCTTCCCNTTTCCNNAANNCNATTANATAAANGNTTTTTCCTTTTTAANAAAAAAATNAANGAGGT
NNNGCGCGNNNNCCCCCCTTCCCAAAAAAAAAAAANTGTAATTTTGGGGGGGGGGGGGGGGNANGN
GTTTTGGGTTTNCTNTTCTTTCNTAGNTNCNTNTTANAAATTTTTNGNGANGCCCCCCCCCCCCCGCCCT
NCCCCCCCNCCCCCNCCANCCCCCNCTTTNNANCTTTTTTTTTTTCCCCCNTNGAAAAAAAAATCCCCT
TCTTTTATGGGGGGGTANNAACANANATTTTTNGGTTNGTGTGCCAACNAAGNNNTTTTTTTTTAGNNG
GGGGGCCTTTTNAAAAAAAATATNGNGNGNGGGGGGCG
TNCTTNTNGNNGGACNCGANCNANCNNNCCCTNNNNTTNAGNCCCTATAGTGGAGTNCGTATTACA
GGGGAGGNCGCTCTAGAACTAGCGGACCCCCCGGGCTGCCANGNTTGCNNGCCCNAGGCCTGGCNNT
CNTGGCNCCTGGTGTGATACGAACGCCNNNNNNNCCCCCTCGGNGANNCTGNCTCCCNTGCNCNTNAC
CACCTGANTNTCCCTNNCNACCNGCCACNCNGNGNTNTTTTNNTNGNGTTNTCCTGTCGNTTGNTCNC
GAGAAATAANCNNTNTNTTTTGNTCGACNGNGANGNCCNNCTCNTTTTCTTTGNTNTNTNNTGNNTCC
CCCCCTNCCCNCNTATTTNGTTTTCCNCNCNCCCNNTNNCNTCGNANTTTNNTACTGCNTNATTCTGTG
NGCCNCAANNCTCCCNNCNTTNNCCGNGNNCNCGNCANNNNNTCCTTNCACTTNTTCNNNNNNCNNC
CCTCCCNGNCCTGNTTNTTCGAANATNCTCGTNGNNNNACNCNTTNNTTNATNNTCTTNNANTCGNNN
CCCCTCNNGCNNNGACGANCGNNNCCTNNNATTNTNTNNGCCNTTTGTTGCNNCNTNCTCGTANGTCN
GTNGCNGNTNGNNCTCCNTNTCNNNNATNCGGNNTCNNTGCTCTTGNNGNCCCNCNNCATCTNNANNT
NNCCTTTNTTNCGNGTGNGTCCNNNTNCCNTCTNTNTNACNNCCNTCCNNNNCNNGCTCNCNTTTTCNT
CCGNCNTCTNTTNTCCNNNNCCCGNNNNGGCCNNGTCNNGTTNTNGNNCNNNCGCNNCTNNCCTNCTNN
NNTTTNNNTNACNNTTANNNANNNNNNCNGTTACTNGCTCTGCCNNCNCCTTTNTNCTCTTCTTCTCCN
NCNGCCCNCCCGCNCCGNCTGCTNCCNCNTCTTTAATTNTTCATCCANCNNNCCGCNCTCGNTCTATCT
TNNCNNTCNANCATNTNGCTTNNCTGTCNGTCCGNCCTNTCGANCTGTNNTATCCNAGCTATTNNATC
NTACTATCCNCTNGTCNGGTCCTTTTTNTTCCTCTTCNTTTNATCTCGCTNTGCNCTGNCANCCNTTN
NATNNTGCGCGCNNNTGTTNNCNTCCTNCNCGCCTCTCTNCTCCTNACTNTNANNCTTCANCNANNT
NNCTTCACNCTCTTATANANNTTCNNTCNNANNCNTNCNTACANTNGNNTCCGGTNNATNNNGAGAGC
GTCTCCGTATAGNCGCGCNTNANNTNTCNCATCGNTCG
TNNANCNANATCTGGAGCTCCACCGCGGTGGCGGCCGCTCTAGAACTAGTGGATCCCCCGGGCTGCAG
GCCCGCGGATCTGTCTCTTGCTTCAACAGTGCTTGGACGGAACAGACCCAGGGACGCCCCTTGTTCAA
GCTCNANCNACCTTCAANCTTTTTTTTCNANCCNAANCNTTGGANNCNANCNNTNNACTNGNCGNGAAC
ANCCGGGANCNACCNNCNTTTTTTAACTTCNTANTANTANCNNNCAATNATNAACNNCCAAAATNNNC
ANAATTANTCTACCNANGGGGANGNCNGCNGCNACCGGCTGGGTAACATGNNACTGGGGGCCTTCTA
CACCTANCTTTCTCTGGGCTTCTATTTCGACCGCGACGANGTGGGCCTGNGAGGGTGTGGGGCACTTTT
TTTCGCGAATTGGCCANGGAGAAACGCCGAGGGGCGCCGGAGCGTNTCTTTGAAACTGCGAAANCAN
CGTGGGGGGCCGCGCCCTCTTTCTGNGCGTGCNCAAACCNTNTCNAAAANAGAGGGGNGAAACCACG
ACGCTNTGGAGGGCGCNCTNTCGTAANAGAANAAAACTNNATAAACCCTGTTGGATNTGCATGGGCT
GGNTTNTGCCCCGGAGAACCCCACATATGTGACTTTCTNGAGNAACACCTTCTATATGANGGAGGGGA
NACTCTTCATNAAAAANGGGNGGACATCTTGNNCAACCCTCNGCANGGTTGGGTGGTGCCCCAAGNT
GGGGGTGGGGGGAAGAATTTTTTTNNNAAANGGGTCNCCCCTCANCCCCAACTAAGGAAGCNTTTGG
GGGAACCACNNGGGCCNTTTTAGNAACCCCCCTTAAAAATAAGGGGGGNGGCTTGGAAAACCCCCT
CTTTTGTAGGNTACTTAAGGGAGGNTTTTTTTTTAAAAAACCTNGGGGGCCCNCTTTTTTAAACCCTTT
GGGGCCATTNTGGGGAAAAAANTAANAAGNTT
TNNANCNANATCTGGAGCTCCACCGCGGTGGCGGCCGCTCTAGAACTAGTGGATCCCCCGGGCTGCAG
GCCCGCGGATCTGTCTCTTGCTTCAACAGTGCTTGGACGGAACAGACCCAGGGACGCCCCTTGTTCAA
GCTCNANCNACCTTCAANCTTTTTTTCNANCCNAANCNTTGGANNCNANCNNTNNACTNGNCGNGAAC
ANCCGGGANCNACCNNCNTTTTTTAACTTCNTANTANTANCNNNCAATNATNAACNNCCAAAATNNNC
ANAATTANTCTACCNANGGGGANGNCNGCNGCNACCGGCTGGGTAACATGNNACTGGGGGCCTTCTA
CACCTANCTTTCTCTGGGCTTCTATTTCGACCGCGACGANGTGGGCCTGNGAGGGTGTGGGGCACTTTT
TTTCGCGAATTGGCCANGGAGAAACGCCGAGGGGCGCCGGAGCGTNTCTTTGAAACTGCGAAANCAN
CGTGGGGGGCCGCGCCCTCTTTCTGNGCGTGCNCAAACCNTNTCNAAAANAGAGGGGNGAAACCACG
ACGCTNTGGAGGGCGCNCTTNTCGTAANAGAANAAAACTNNATAAACCCTGTTGGATNTGCATGGGCT
GGNTTTNTGCCCCGGAGAACCCCACATATGTGACTTTCTNGAGNAACACCTTCTATATGANGGAGGGGA
NACTCTTCATNAAAAANGGGNGGACATCTTGNNCAACCCTCNGCANGGTTGGGTGGTGCCCCAAGNT
GGGGGTGGGGGGAAGAATTTTTTTNNNAAANGGGTCNCCCCTCANCCCCAACTAAGGAAGCNTTTTGG
GGGAACCACNNGGGCCNTTTTAGNAAACCCCCCTTAAAAATAAGGGGGGNGGCTTGGAAAACCCCCT
CTTTTGTAGGNTACTTAAGGGAGGNTTTTTTTTTAAAAAACCTNGGGGGCCCNCTTTTTTAAACCCTTT
GGGGCCATTNTGGGGAAAAAANTAANAAGNTT
TNNNNAAAAGCTGGAGCTCCACCGCGGTGGCGGCCGCTCTAGAACTAGTGGATCCCCCGGGCTGCAG
GAATTCGGCACGAGGCACTAATAAAAATTGTAAACAATGCATTCATCGACCTTCCAGCCCCATCAAAC
ATTTCATCATGATGAAATTTCGGTTCCCTCCTGGGAATCTGCCTAATCCTACAAATCCTCACAGGCCTA
TTCNTAGCAATACACTACACATCCGACACAACAACAGCNTTCTCCTCTGTTACCCATATCTGCCGAGAC
GTGACCTACGGCTGAAATATTCCGATACATACACGCCAACNGAGCTTNNATGGTTTTTATCTGGNTAT
ATATGCCCGGAGGAACAAGNTTATATTACCGGGTCNTAGCCTTTTCTAAAAACATNGAATATTNGAGT
NATTCNTTTGNTTACANNNATAAGCCCNNNANTTAAANGGANTCCNCCTACCNTNAAGGNCAATANTA
NTNTNNNGNACCACAGNCNTTAACCANCTTTTAATAANCATTCCATNNCNTNGNACCAAATTAAGCGN
AAGGAATNGNGGNGGATTTTAANAAAACAAAGCACCCCTTACCCAATNNTTGGNTTTCCATTTAATC
CTTCNNTTAATNATCATACCAATGGCCTTAGNCCACTTATTTTTCTTCNCGNAAAANGGTTCCAACAAA
CCCAACAGGAANTTTCTTANAACGTNNANCAAATCCCNTTCCNCCCCTTCTTTTTCCNTTAAGGGCCN
TTTTAGGGGNCCCTTTTACNAATTTAAGCCTTAAAACCCCCNAGGCCCTTTTCCNCCCCCGACCCTCCT
TGGGGAACCCCANAAACTTACCCCCCAAGCCAATTCCATTTAAAAAACCCCCCNTTTAAATTAAAACC
CGGNGGGGAACCTTTTTTTTTTGGGAAACCCAANTTTTAAAAACAATNCCCCCCCAAACCTTGGGGGG
GG
TTGANANNGCTGGACTCCNCCGCGGTGGCGGCCGCTCTAGAACTAGTGGATCNCCCGGGCTGCAGGA
NTTNGGCTCNAGGATTTTNNNCTGNTNCCANNCATGCAACTTGAANATGGGNNNCTNNGTTGAACGAT
CCACTATGGAAANGGAAAGNGNNGNCANCTGGTGTNTCTTACATGGANGGNGCTCTAGNANATTCCC
ACCTGCGTACTGNAGAATNCCNATGNTACAACNTAGATCATACATGNCGNTANCTANNANANCANCT
NGCNAGTTGANNATGAATNNANTCNNNNTGGNCANNNACNNAATANGATNTNNTGNNNNTGTATNT
AATNNTAAGATTNNACTTANNCTNCNANNTTNCNNNNAGTANNCTNCNTACTTNTNNGGNANCNGCN
NTNNTNANTACCNANNTTNTTNGNGTNACTCNAANTNNTGNTNCANTTNNTGNNTANCCNCTNNANTC
CTCATCCANCTNTNTGATANNNCNTTTGCCCCNATACCNTNTTNTCTTTGCNNNTTTCTTGNCTCNATN
TNAACNCTTTGATCCNAATNNANNTAACNNANTNCTATNNCNNCNANCNCNNATNTCCCTCTNCTTNT
ACTGTTTNNTNNTNAATTNATNTNNCNNGNCCNCNCANCCTTTTCNNNTATCTCTTCNGTCTNTTCTNA
TACNCTTNGCTNNCTTTNCCTNCTGTCTCCNANTTTCCCTNCTCCATNTCNTTTTTGACCCNTGNNCNTC
CCCCNNANNTTCNNTCAGNAACNTTCCTTNNNAANTTTNTTTATTCTNCNTTNTNTTTCTTACNCCTNT
NTCNCTTTCTANTTTNTNAGNCNCNCTCCCATNTTNNTTTTNNNCTCNANAATNNCCANATNTNNTTCC
CNTNTTGTANTNTNCCCACCNCNTCTNANTNTNNCTTNNTTCTGNNTCACTNTNNTNNCNNCTCTNNNT
TNTNNTNNNCTTNCTCGACNTTCCTACAGTCCTCNCTNNTNNTTTNTTCNTNCTATCTTATATTNCTNTC
ANNTCNCTGNNNCNNCTNTGTNNTNCGCTCTTCNTCTANNNCTAGATAGTGANCTCATCNTCTAATTTT
CTNCTTTCCCTCACCT
NAAAAGCTGGAGCTCCACCGCGGTGGCGGCCGCTCTAGAACTAGTGGATCCCCCGGGCTGCAGGTAG
CTCCTCTTCATCTCCCCAGACCGGCCAGCAGGAACCCCCAAAGCCTNCCTGAGTCTGACAGCCAGGAC
GTGGGGCTGAGGGCCGGGAACAGCTTCCCCCAGGCAGCTCCTGCCAGGCTGTCCCGTGGATGCTTCAC
TTGCCTTTTTCCTCCTCTCCCAGAACAGTATTTGGGAGATTTTACTACTTATTCAGACTCCTGGTTATT
ATTGCAGATTGGCAAGTGCTTGTTTGGGGGATGAAGGGTAGGGCTCAGAAAGTGGAAATATGGTCTTC
CTGACCTTTCAGGTCCTTCAGCATCCTCTTCTCCCTGCCTCTGGGACCTTCCTGGTTTTCCAGGAGATCC
TGGCTGGAAGGGCTCTGAGCTCTGTAATAGAGCTGGGTCTAAGGGGAACTGGTGTGTAAAGACCCTGG
CATGGGGCAGAAACACACTGCTTGCCAGCAGCCTGGTCATCCTCTTCCCAGAACTTGTTGGCTGGTAG
TTCACTCCCAGCTGGATCCCTTCAGAGCCTGAGTCTTGGGCCACAGTCTGGGACCCAGCAGACACTGC
CCAGGACCCTGATGCATTCTNTTNTCGCCTGTCCCTNTGCCCTTCCTTCCTGGACCATACTACTTGGAAT
CACTGNAATCTGAATTCGCCTTAATTGGCANGGCCTGGGTCCAGTGCCCCTNTCCTTGGNCCTTGNAAG
AAAGGGCAAAAANAGCNCANGGGACCCCTTNCCCAGGNTAANAACCGAGCTTCCCCCAATNGGGAAG
GAACCTGCCGGTNGGCAAATTCCCCCCTTGNCCCAACCCCAAAAATTTTTAANGGGANGGNCCCCAA
NAAACCCNNAAAACTNCCCANGGNTTTCTTAANTTGGGAACCTNCCCCCCCNGACNTTTTGGACCTTG
TCCNANAAAGGACN
TNNNACAAAGCTGGAGCTCCCCGCGGTGGCGGCCGCTCTAGAACTAGTGGATCCCCCGGGCTGGAGG
AANTNGNCNCGAGGCATGGCTGCAGTCACCATCTACAGTGATTTTGGAGGCCCCCAAAAATAAAGNCA
GCCACAATTCCCATTTATTNGCGATCAAGTGATGGGACCAGATGCCATGATCTTAGTTTTCTGAATGTT
GAGCTTTAAGCCAACTTTTTCACTCTCCACTTTCACTTTCATCAAGAGGCTCTTTAGTTCTTCTTCACTT
TCTGCCATAAGGGTAGTGTCATCTGCATATCTGAGGTTATTGATATTTCTCTCGGCAATCTCGATACCA
GCTTGTGCTTTCTCCAGCCCAGCATTTCTCATGATGTACTCTGCATATAAGTTAAATAAACAGGGTGAC
AATAGACAGCCTTGATGTACTCCTTTTCCAATTTGGACCCAGTCTGTTGTTCCATGTCCAGTTCTAACTG
TTGCTTCCTGACCTGCATATAGGTTTCTCAAGAGGCACATCAGGTGGTCTGGTATCCCATCTCTTTCAG
AATTTTCCATAAGTTTATTGTGATCCACACAAGTCAAAAGCCTTTGGCATAATCAATAAAGCAAAAAT
AGATGTTTTTCTGGAACTCTCTTGCTTTTCCAATGGATCCAACGGATGTAATGGTTGAANAAACTGAAC
GGNTCTATGANGACCTAAAAAGACCTTNTAAAACTAACACCCAAAAAAGAAGGCCCTTTTGGTTATAN
GGGACTGGAATGCCAAAAGTAGGAAAGCAAGAAACCCCTGGAGTAACANGCAAANTTNGGCCTTGGA
GTNCCNAAAGGAAGCNGGGCCAAAGGCTTACCAAAANTTTGCCAANAAAAACNCCCTGGGNCATAAA
CAAACACCCTTTTTCCAAN
GCCTATANNGAGNNGNATNACAGTGGCGGCCGCTCTAGAACTAGTGGATCCCCCGGGCTGCAGGNAC
CATCTANCNNGANTGNNGNTGANCGGNCNNGTNNTGAGNAAANTNANNNCNGAGAAGACCNTGGTN
GTNGNNTTGCTGCTCATTGACGTTGNAGNNCTTGAACGAAGCTNCNTGGCTTGTTGCCCTNACATTAAT
NNAANGNGNAAAGNNNTATNANCACNCACTGCNGTNCATACNANNNNNGCNCANNNGAGCTCTNAC
CANCAAGNNNNTNNNGGATNTGCAGAANNNTCTNTCNAANNNGCNNNNGNACTACANANACNNNNN
NGNACTTAGANNNAANTGCNGCATNCANTGNAACNANNNGGCNTNATGCNANNNNNTGGANNNGGA
ACCNNACANNCGATGGAATNNGANNTNCNTAGANNCGANAGCCCNNTTGACTANTTGAATAAGGCNN
NTGNACCTTTCATAAAGANNGTNGAAAAGCNGATNTACNAGGCCATATCGTGNTGGCTNGAATNGTA
CANNCTGCTCNTGTTATGATAAACCTATTNCAATACTAAATAACCTAATTCNTGCTGCTCATACATNTA
ANATGCTGCTTTATACTTTGNNCCANGCTGACCCNTTCNTGAATATCCATNCANNNTTAGGGNCATGC
CNTNNAATCTTNTTNTCCTTGNCCAGAAGGCGNTACCANATNTNNCAATANAATTTAGCATGGACTA
GGAGGCCCTGAATNACCCANGGACATTACGNNGGANNTTGTTAATNCCATAATNGAANCNACTNCCN
NGTGNAACNATGANCNCTNNCGANCCNTANGNTGGTTNTNGCCNTAGCCNNAATTTCNCNATNCAA
ATTTGGNCCNNTTNAAGGNCTTTTTGCTCCTAATCTNNTNAACANTTCCCNCTTTCANACNANTTTNNA
NTNATANTTTCNTNGCCNTTTAANTCNNCCANNNNTTTNCGGGANNNNCTAAATNNTCCTANTGTTAA
GNCCGTTTTNTTTNGGGCCC
GCCTACNGGAGCTNCACCGCGGTGGCGGCCGCTCTAGAACTAGTGGATCCCCCGGGCTGGCAGGGTTT
TTTTTTTTTTTTTTTTTAAGTTTTTAAACCTTTTTTTANGGGGANCCNGNCTTTTTTNAAAAANCCCCCC
CCAAANCCCCCCANGNANCNTAANNNTTCCNTTTTTNCCCCCCNAANGAAGGGCANAANNCNTTNNA
AAANTTGGGNGNAANCCCAAAAANTTTTTNAAAGGGCCNANANTTTTTTTTTTNTNNAAANGAAAAN
NCCCCCNNAAAGGGNCNTTTTTTCCCCCCCCCCCNNCNNCNNGNNAAANTTTTTNGNAAAGNCCCCCC
TTTTAACCAAANCAAACNAAANTNCNNGANNGGGNCNGNAANNCCNNANNGGGGGGGTAACCCNNG
GNACCGGCCNAGNATTNAAAAAANTTTTTTAAACTTTTGNAACCNTTTTTTTTNTTTTTTAAGAANNGG
GTTCCNCNNTCNGGCCCTTNNCCCNNTTAAAAANTNCCAAAANAAAAAAGGCGGNNNANTNGGGTNT
TNNACCCAAAAANCTNNTTTGGAAAAAAAAGGCNTNNCCAAAATTTTTTNCCAAACNTTGNAAAAAT
NTGGNAAGGGGNTNTTTTTTTTTNAAAAAAAAAACTTTTNTTTGGGGGGGGNCCTCCCGAATTTTNTN
NGGGGGTGNGTTTTTCNGCCCCNANNAANAAAACCCCCCCCNGGNGGAATTTCNCCGGAGNAAACAN
AAAAAAGGGGGNNTTTTTTTNAAAACCNGGGGGGGGGGCNCCCCTCCAAAAAAAAAANGGGGGGGG
GGNGGGGGGNGAAANNTTGGGNCCCTTTTNCCGNGGCNNNAANTTTTTNNTTTTNCCNCGANNGGCN
CNCCCCNGGNNGGNTTTNNGGAATTTTTTTCCCTTAAAAAAANNNCCCNGGGGGGGCACNNNCCTTNC
ACNNNNCCCNGGGNTTCCCCCGCCCCCCNAAANNACGGGGNGGGC
NGTNNCNTAGGGNNANGNNTNCCTCNTCCNCTCCCNNNTCTTNAGNCCCTATAGTGGAGTGCGTATTA
CAANGGNGGACGCTCTAGCNNCTAGNGGCATCCCCCCGGGCCNGCAGGCNATCCTNCTCTAGGTNNC
NNTTNNNNTNGACTCGGGTNNTNCANCGCCTTNCTNNAGTTCTTNTNCCCCGCGGATGANNACTGNNT
CCCATCTGNNTANTACCNCANGNNNTNCGCCTTNACNTGGNCAGNCCNCNGGCCCNCGATCGNNTGA
NACAAGTGNGCTCTNNTGGTTGCNGGACNANCACACTGCNTNNNANTNTCNCNTGNCACNCGCTNAN
ACNNTNANANNGANTTTGCTGGNACANGCANTGNCCGTCATNNCNTNTNTGCNNGGGCCAGTANNAG
NANTGGANACNNNNTTNCNCCGNAGCTTGTNTANGCCCAANTTCTTNNTNCTACCAGGGAGCNNANA
ANGNGGCCTCTTNTGNTANNCAAGGNGNNTGNNATNNTGGGNCNCCGNCNNCCNNGTCCCGATGNGG
CACCTNCTNTNNTATTANGGTCATNTGACNCNCCCCTGGGCGAACTTCTGAATCNNNNNNGNNAGNN
TCCTNGGGACGCCNTAANANTNTANNCCCCCCNNTNNCTAGNAGAGGNNGNTNCCNANNACCNGCCT
NANGGGCNGANCNCGNNNGNANNNNANCNCGTNNNCCATNTCNCCGCCATTNGTCTNGNNAANGCTT
ACTCNNCANNNNACCTCGNCTGTCTTNNCTNCCNTGNGCCNNGGNGNNGATCGCTNACNNGNNCNCC
GGCAAGCCCNGCNGNNNNNGNNNNNTNTAANTNNCCCTGNTGCCCNNNNTNNNGNTTNCCNNATGTC
NACNNCCATTNNNNTCCNNNCAGNNGCNNCNNCNNGTTTTGNTCCCTTCCTTATGTGTCCCCTCTTCTT
GCCCCCCNCCNTCGCAATNGGANCNNTTGGCCNNNTGNNTATCANNTNCTCANTNNGGNANCCATACC
CACNGANCACNTAGAANNGTATATCTCANCNCNGNGCATTANTTNNNTTANCTTNCTCNTNNGANTTN
TCTACTNCGCTCTCATNNNTGCCNTCNCNTATTNTCATTAGNNCANAANCACNNTGNTGANTNCNNTC
NNNTNNCCNCTCATANNNNNNCANCNTNCAGTCTCNGNCCACANNGCNAGCGCTGTTNNNCNCNTGC
GNANNCANNTNTNTNTCGTANCANTGANANGANGNTTATNNACTGTTTGTGTTCTATCNAGNGCGNN
NCGCNCACANGNGGCTGNNACNNACATANANANNTCNGCTCATNCGATGGNGNNGCCCTANCGANTG
NNNANGCNCGCG
AACAAAACTGGAGCTCCACCGCGGTGGCGGCCGCTCTAGAACTAGTGGATCCCCCGGGCTGCAGGGTT
NAGNGTCCTCTTCAGCGACTCAGTACGGGGCTCATGAGCAGACACATGCGATGTATGTTTCNACTGGT
TCCTTNGTTAANAANNANNCNNNCCTNANGGCNACCTTGANCCNNNNNNTTCANANTCTTAANCNTTA
ANTNNCCCCANCGGANNNNAANNAANNCNNNATGGGGGGAACCATTCTNGGCCCAANNCTNGTNAA
CANCATTAACANCANGGCGGTTAAGNTTTTCATTTGGGCANNCCACANNANCANNCTGGCATTTAACA
TGNNGGGGTTGGCCAANCCCACCTTTATNAACCTGGCTAATANACACAGTCTACAGAAATATTTTTC
CCAACAGNACANNCAGACACGTGTTTTACAATATCATACTNTTCATGTGTAAGANCGGGGNTNCACNN
AATNTACACCACATNGGGCATGTGCNTCNGAACAANCCCACCCCCAACAANNNNNTGTGAAGGGNGT
CTGGGGGAAACCNAAAGGGGAAAATCCTCTTCCTTTTATGGGTNTACAACTGGGAGCACANAAAACT
AGAAATTCTTNTTTTTGTTTGNAAAAANATATNTGGATNNNTTGTACAACCCATAANAAGNNTGACAA
CTGACTTGTCGGNGGAAGAAAATTTGGACCNCCGTAGAAGGCCTTTTTTGAAAATTGTGGGNNGNTGC
ATAATAACAAAATGATGGTTGTCCAAAAACCCCCAAGGAACCCCAACNTTTGTTGGTGGNAAANTGG
GAGATTTTTTTTTTTTTATTGACCCCTTGAAAGGGNTTGANAAAACAAAATCATTTTTNTNAAAAAAAA
AGTANCCANNAGGGGGTTTTTGGTTGGTTTTTACCGCCTNNAAAAAAAAAAAAAAAAAATTCNNGGG
GGGGGGGNN
AACAAAACTGGAGCTCCACCGCGGTGGCGGCCGCTCTAGAACTAGTGGATCCCCCGGGCTGCAGGGTT
NAGNGTCCTCTTCAGCGACTCAGTACGGGGCTCATGAGCAGACACATGCGATGTATGTTTCNACTGGT
TCCTTNGTTAANAANNANNCNNNCCTNANGGCNACCTTGANCCNNNNNNTTCANANTCTTAANCNTTA
ANTNNCCCCANCGGANNNNAANNAANNCNNNATGGGGGGAACCATTCTNGGCCCAANNCTNGTNAA
CANCATTAACANCANGGCGGTTAAGNTTTTCATTTGGGCANNCCACANNANCANNCTGGCATTTAACA
TGNNGGGGTTGGCCAANCCCACCTTTATNAACCTGGCTTAATANACACAGTCTACAGAAATATTTTTC
CCAACAGNACANNCAGACACGTGTTTTACAATATCATACTNTTCATGTGTAAGANCGGGGNTNCACNN
AATNTACACCACATNGGGCATGTGCNTCNGAACAANCCCACCCCCAACAANNNNNTGTGAAGGGNGT
CTGGGGGAAACCNAAAGGGGAAAATCCTCTTCCTTTTATGGGTNTACAACTGGGAGCACANAAAACT
AGAAATTCTTNTTTTTGTTTGNAAAAANATATNTGGATNNNTTGTACAACCCATAANAAGNNTGACAA
CTGACTTGTCGGNGGAAGAAAATTTGGACCNCCGTAGAAGGCCTTTTTTGAAAATTGTGGGNNGNTGC
ATAATAACAAAATGATGGTTGTCCAAAAACCCCCAAGGAACCCCAACNTTTGTTGGTGGNAAANTGG
GAGATTTTTTTTTTTTTATTGACCCCTTGAAAGGGNTTGANAAAACAAAATCATTTTNTNAAAAAAAA
AGTANCCANNAGGGGGTTTTTGGTTGGTTTTTACCGCCTNNAAAAAAAAAAAAAAAAAATTCNNGGG
GGGGGGGNN
TTNAAAAAGCTGGAGCTCCACCGCGGTGGCGGCCGCTCTAGAACTAGTGGATCCCCCGGGCTGCAGGT
TACACCCCACCCCCCAACATAAGGACAAGTCAGTTTGTCAGTAGCTTCCTCTGGCTTGAACCCCCTCGA
TTTTTATAGCCCACTTAACCATGCATAACAGACAAGTCCCATATTTGTCAGTAGATGCCTTTTTTTTTTT
TNCCCCCCNNGGNTTAANCCTTAAGGGCCNAANCNNAAAAAAAAAANNCNNANCGGTTTANAAAANC
NACTTAGGGCCTTGGAATNNAACTTTGGCNTTGGNAANANATTACCTTTTNAGGNCCAAAGGGCCCCC
GGGNNCCNCCCNAAAACCTTTGCCCCGGGGNGGGGGNGGGGGGGGGGGCATTTAAAAANANNANNT
CCNTGCATNGGAAAAAAAATNGCNCTTTTCNTTAATTTAAATTTCCAAAAANTTTNCTTTTTNCCTTTC
CGGGGGAGCCCCCACCCCCCCTTTGGGAANGTTNTTNCCAAATTTGGTTTTTTNGGGGAAACCTGNTTT
GAAATTNAAAAACNTTTTTTTTTCCCAACCTGAAACCTTTGGGGGAAAAAATTTTGCCNGGGTTNNGG
NANGGNCNNNNAAAAAGGAAAAATGGNAAAACGGGGGANAAAAATTGGGNAAANAAAGGGGGAAC
TTTGGGAAAAAAAATTTTTTTTTTTTTTTAAAGGGGGGGGGGAAGGGGGAACTTTAANGGGNTTTTTA
AAAAAAATTTTGTTTTTGGGGGGNNAAAANNNCCCCCCNTTNTTNNTGGGGGAATTTTTTNAAAAAAN
NCCCCCCCCAANNGGGGGGGAAAAGGTTTTNAAAAAAAAAAAACTTTTTNNGGGGGGGGGGAACNN
NAAAAAGGGGGTTTTTTTTNCCCAAAAAAAAAATTTTNGGGGNAAAAANAAATTTTTTTGGNAAAAA
AANNTTTTAANGGGGGTTTTNGCCNNAAAAAAAACCGGGGGGNT
TTGANACAGCTGGACTCCCCGCGGTGGCGGCCGCTCTAGAACTAGTGGATCCCCCGGGCTGCAGGGCA
CCCCGTGCTGCTGACGGAGGCCCCTCTGAACCCCAAGGCCAACCGTGAGAAGATGACCCAGATCATGT
TCGAGACGTTCAACACTCCTGCCATGTATGTGGCCATCCAGGCTGTGCTGTCTCTGTACGCCTCTGGCC
GCACCACTGGCATTGTCATGGACTCTGGGGACGGGGTCACCCACACGGTGCCCATCTACGAGGGGTAC
GCCCTCCCCCACGCCATCCTGCGTCTGGACCTGGCTGGCGGGGACCTGACAGACTACCTCATGAAGAT
CCTCACGGAACGTGGTTACAGCTTTACCACCACAGCCGAGCGGGAAATTGTGCGTGACATCAAGGAGA
AGCTCTGCTACGTCGCCCTGGACTTCGAGCAGGAGATGGCTACCGCTGCATCCTCCTCCTCCTTGGAGA
ANACTATGAGCTGCCCGATGGTCANGTGATCACCATTGGCAATGAGCGGTTCCGGTGTCCCGANGCAC
TCTTTCAGCCGTCCTTCTTGGGTATGGAGTCCTGGNGCATNCATGAAANCACCTTCAACTTCATCATGA
AGTGNGACGTGGACATCCGAAAGGACCTGTATGCCACACGGNGCTTGTTNGNGGGAACACCATTGTA
CCCTGGCTTCNTTGACAGGATCAAAAGGAAATACCGGGGCTGGGTTCCAAGCCCGATGAAANATCAA
GAATCTTTGGTTCTTCCCGAACGCCAAGTACCTCGGGGGGGGATCGGGANGGTTCCATTCTTGGCCTTC
CCTGGNCCNCCTTTCAAAAANAAGGNGGGTTCAACCCANCCCGGAANTTTGACCAAGTNTTGGGCCCC
CTCCNTTTGGCCCCCCCGAAAANGCCTTTTTAAAAGGGACTNGGGAACCCAANAGCCTTTANANTTT
AAAAAGCTGGAGCTCCACCGCGGTGGCGGCCGCTCTAGAACTAGTGGATCCCCCGGGCTGCAGGAATT
CGGCACGAGGCAGCCTTGTACCTTCATCACAACGAGATCCAGGAAGTGGGCAGTTCTATGAAAGGCCT
CCGATCATTGATCTTGCTGGACCTGAGCTACAACCACCTTAGGAAGGTACCTGATGGACTGCCCTCAG
CCCTTGAGCAGCTGTACCTGGAGCACAACAACGTCTTCTCAGTCCCCGACAGCTACTTCCGGGGGTCA
CCCAAGCTGCTGTATGTGCGGCTATCCCACAACAGCCTCACCAACAATGGCCTGGCCTCAAATACCTT
CAATTCCAGCAGCCTCCTTGAGCTGACCTCTCCTACAACCAGCTGGAGAAGATCCCCCCAGTCAGCAC
CAACCTGGAGAACCTCTACCTCCAAGGCAATAGGATCAATGAGTTCTCCATCAGCAGCTTCTGCACCG
GGGTGGATGTCATGAACTTCTCCAAGCTGCAGGTGCTGCGCCTGGATGGCAACGAGATCAAGCGCAGC
GCGATGCCCGNTGACGCGCCCCTNTGCCTGCGCCTGGCTAGCCTCATGGAGATCTGAGCGCCACTGGG
CGCANGGCCATGCCCCCACGCCTNTTTGCATTTTGGCTTGATGGTTGGGTTTGGCTTTTGAATGGAAGG
TCTGGGAACAANACCGTGTGACAAAAGGTCCATNGGGCTCTNTTTTTAAACCTTNTTNCCTGGAGGGC
AGTTTTAGGGGGGGNCANGGGGAAAGGCCCCTTTTTTTGTTGGGGGGGAAAATGAANCCCTTCCGTTT
TCCAAAAAAAAAAAAANGGTTTGGGAAAAAAGGGGTNAAAACCCCTGGAAAGTCCCCAGTCCCCCCG
AAAANTTTNNTTTACCCCTTAANGGGTTTTTTCAAAGGGGAATCCAAGNNGCCTTGNAACCCNTTGGC
CTNGGAANNAANAAAAAAAAAACTGN
AAAGCTGGAGCTCCCCGCGGTGGCGGCCGCTCTAGAACTAGTGGATCCCCCGGGCTGCAGGGTAAACC
CAAGCCCTTGACCTCTTACAGGAGCTTTGTCTGCCCTCTTAATAACATCCGGCCTAACCATGTGATTTC
ACTTTAACTCAATGACCCTGCTAATAATTGGCCTAACAACAAATATACTAACAATATACCAATGATGA
CGAGATGTTATCCGAGAAAGCACCTTCCAAGGGCACCATACCCCAGCTGTCCAAAAAGGCCTCCGTTA
TGGAATAATTCTTTTTATTATCTCCGAAGTACTATTCTTTACCGGTTTTTCTGAGCTTTCTACCACTCA
AGCCTCGCCCCCACCCCTGAACTAGGCGGCTGCTGACCCCCAACAGGCATTCACCCACTAAACCCCCT
AGAAGTCCCACTGCTCAACACCTCTGTCCTATTGGCTTCCGGAGTTTCTATTACCTGAGCCCATCATAG
TTTAATAGAAGGGGACCGAAAGCATATATTACAAGCCCTATTTATCACCATCACATTAGGAGTCTACT
TCACACTACTACAAGCCTCAGAATACTATGAAGCACCTTTTACTATCTCCGACGGAGTTTACGGCTCAA
CTTTTTTTGTAGCCACAGGCTTNCACGGNCTTCCACGTCATCANTGGGGTCCACCTTCTTAATTGGCTG
GCTTCTTTCCGCCAATTAAAANTTCANTTACTTCTAACCACCACTTTNGGTTTTGAAAGCCCCTGGCCT
GGAACTGGAANTTTCGGAAAACGTANNCCGGACTTTTCCCTCTATGGTNCTATCCAATGGGGGAAGGC
TCCTNAAAANNNANNNNNNNNNNNNNNNNNNNNNNNNNNNNNNNNANNNNNNNNNNNNNNNNNNN
NNNNNNNAAAAAANGNGGGGGGGGG
AAAGCTGGAGCTCCACCGCGGTGGCGGCCGCTCTAGAACTAGTGGATCCCCCGGGCTGCAGGCCCGA
GCGCCGCTCTGGCCGCACTGCGCTCGCCCTGAGCTCCGGGCTCCTGCTAAGCCAGCGCCGCTGTCGCCT
CCCTCCAGTCGCCATCATGATCATCTACCGGGACCTCATTAGCCATGACGAGATGTTCTCCGACATCTA
CAAGATCCGGGAGGTCGCGGACGGGCTGTGTCTGGAGGTGGAGGGGAAGATGGTCAGTAGGACAGAG
GGTAACATCGATGACTCGCTCATTGGTGGAAATGCCTCCGGTGAAGGCCGCGAGGGCGAAGGTACCGA
AAGCACAGTAATCACTGGTGTCGATATTGTCATGAACCATCACTTGCAGGAAACCAGCTTCACAAAAG
AAGCCTACAAGAAGTACATCAAAGATTACATGAAGTCAATCAAAGGGAAACTTGAAGAACAGAGACC
AGAAAGAGTAAAACCTTTTATGACAGGGGCTGCAGAACAAATCAAGGACATCCTTGCTAATTTCAAAA
ACTATCAGTTCTTTATTGGTGAAAACATGAATCCAGATGGGATGGTTGCTCTGCTGGACTACCGTGAGG
ATGGTGTAACCCCATATATGATTTTCTTTAAGGATGGTTTAGAGATGGAAAAATGTTAACAAAGTTGG
CAGTTACTTTGGATCAATCACCTGTCGTCATAACTGGCTGGCTACTGCTTTTCATCCACACAACACCAG
GACTTANACAGATGGGACTGATGTCATCTCGAGCTCTTTCATTTTGTTTTTGAACCGTTTGATTTATTTT
GGAAGCGGANGCATTTGGTTTTTGAAAAAAACGTGGTCNTTGTAGGTTGNCTAAAAAATAAAACCGCA
NTTTAACTCAAAAAAAAAAAAAAAAAAAANTTCGNGGGGGGGGCCGGNTCCCCAATTNNCCCTTTTT
NNGGNGGNNNNNTNTTTTATNNNNNNNNNNNNNNNNNNC
NCCTATNNGGAGNNNCATNNCAGTGGCGGCCGCTCTAGAAGTAGTGGATCCCCGGGGCTGCAGGAAT
TCNGNCCCGAGGCTTAATCAGTGGTGGGAAGAACGGTCTCAGAACTGTTTGTCTCAATTGGCCATTTA
AGTTTAATAGTAAAANACTGGTTAATGATAACAATGCNTCGTAAAACCTTCANAAGGAAAGGANAAT
GTTTTTGTGGACCATATGTTTTGNGNGTGGCAGTTTAAGTTATTAGTTTTTAAAATCAGTNCTTTTTAAT
GAAANCAACTTGACCAAAAATCTGTCACANAATTTGAAACCCATTAAAAAAAAAAAAAAAAAACNNG
GGGGGGGGCCCNGGCCCCNATTNCCCCNTATNGGGNGNNTNTTNACANNNNNNNCNNNNNNNNNTNN
NNNNNCNTNTNNNNNTNNNNTCTNNCNNNNNCNNNTCNNNNCNNNNNCNNANNNTNCNNCNTTCNN
NNNNNNNNNNNNNNTNCNNNNCTNTTTCNNNNNNCNNNNNNNNNNTTCCNNNTNCTNTNNTCNNNCC
NNNTNCCNNTNACCTNNTCNNNNNTCNNNCTCTNANNNNNTNTNNNNNCCNNNNCCNNNCTNTT
NNNGNCNNANNNNNTTNNCNNNNNCNNNNNNTNCTNNNNTNCCCTTNNCNCNNNTTNNTNNTCCNNN
NNCCNNTNTNTNNTNCNNCNNCNNNTCCNTCNNNTNTNTNTNNNNNNNTNCCTNNNNTNTCNAN
NNTNTCNNTCTCCNNNNCTNNCNNTNNNNNTTCNNNCTNTTNCNNNNTCNNNNNNNCCCNNNCTCNTN
NNNNNTCNTTNNCTTNTNNNNNNTNNNNNNNTCNNNNNCCTCNNNCNNCACNNTCTTTNNNTANNTN
NNNCTCNNNNNTNTTNNTCNTNNNNTNNNTNNNTNCCNNNNNNNTTACCTNNCCTCNNNNNTNTN
CTNTNNNNNNNNNTNTNNCNNNNNNNNCCNTNTNCCNNNTNNNCTTCTNTNTNNNNNNNTCNNNNNN
NTNNTCCTCN
TCGNGNTAGGGGNGGATNGCNGNNATNGCCTCTNTTACCCANTCGNAGNCNTAATNNGGAGNCTNCA
CCNCAGTGGCAGGCACGCTGCTNGNANCNAGTGNGTNCTCAAANCGGTCATGCNCTAAATGNCCGCT
CGAGGCCATTTTTTNTCTNTNTNACNTNGGCGNTATNCTGNTCCGGNGCNGNNGAGGCCNCNACCTGN
AGAAGCANCNGTNNTTGNTCTNATCAGAACTTTNANTANNCCGGCTGTNGTGNACNANNTTACAGTNG
GNGTGNTACTGCNANTNTNNGGCATTTNTNGANTCGTCNCANNCTAAGGTATAGAANGNCGGNGTCGT
ATTANTGTCNTTGTTAATGGACNAAGTNNCGNCNNAGGGANTACNCGGCTTTANGGGCNGGTTGGAN
NCTNAANCCTTNGNTNNANTGNTTNGGNCNCNATCCNNTGTATNNGNNTCCNNTAGTNTNCNTCNNNN
GNTTTCTTGNNAANTGNGNCNATCTACTNAGTNCGGCCNGGGNGGATANCTNTNTNANCTGTTTACGC
TCTNNGACNCCGNNNNNTGTCAAAGTTTTGGANTGCCTNNTACTCCCANNCANGATGNGCGNNTNNAC
CNCCCAANTNTNNNGTAGGNACCCANCCNNGAGCACTNTNTTNTNCNTTAANNNTCAGCNCGCNCTNG
NNCAGGGTNTTNNNNANANGNTGGGNNNCCNNACNTNACAGTGGATNCTNACATCTGTCNCCACTNC
TTGANNNTGNNCNTTTGTNCCGGGNAGNTTNACNCNTGNGGGGNTNAAAGGNATNTTNCNTACCACN
TGNNTNTCTGGANTGGCGCNCNTTACAAATCTGNNAGTNNCGCTTGNGGNNACCNCCAGGGGNAAAN
TTNNNNNNGNTTGNGNNTNANTNGGNAANGCNCNTTCNCATNTNNCCCCCGNATACCCNNTTTACTNT
TTCNTTTTACTTATAANTTTNTACTNCTATGANNTTCACCNNGNNCTAGCAATNANAATAGNGCNTAN
NTNTCAGGNGCNACCNGCCNNTGGGTCGANTCTNTCANNATATCGCTANNANTATATNTCNGCANAN
NCTTCTNGCGGAAACNTATCGGCGCGNNTNANNNNTGTATCTNCNTNNNCANATAACTCTNAGTTGCN
GTGCNGNGTNNTGTTANNCTANATNNNCNGTGTAAATNNCNGCNCANTCCNNTGGTNCCGTANGGTCT
TNGAGTACAACTANTGCNNCNGTTNGCTTGGACGTTCTCNNCNTANNNCTGTAGNANAANNTCGCAGN
TANTNNTTNANACNNGTATAGANAATTACTCNCATTATCGNNTNANNNATNGANATTNGTNGTNCGTC
TNNNTANATTCGANGANTNTTGCANGNTTNTNTNATANATAGACGATAGNGTNANGAGTCN
NCAAAANCTGGAGCTCCACCGCGGTGGCGGCCGCTCTAGAACTAGTGGATCCCCCGGGCTGCAGGAA
NTTCGGCACGAGGCCTGGAGCATGAAGCCCGGAACTGCATGAACCGCTCAAAACAGGTCTTCACGTGC
TCTGTGTGCCAGGAAACGTTCCGCCGNAGGATGGANNTGCGGNNGCNCNTGGNGTCCNANNCGGGGN
AAANCCTTACAAGNNNTCNTCNTGTTCCNANNANTTANTNCAAAAAANGGACNNGCAAANCCNNNT
NATNAACTTGNNCGNACCCCCNANCCCCATNNCTNGNCCAACNNGNNCNANNGNNTTCTNGTCCNGA
NCGNACTTGNACTNGCCAAGGGTTNAANCACCNTGNGGGAGAAATGTTTGTGTGCCAAGAGATGCGC
GGCACACNGCATCTAGCCCCGNATGTGGNTTGNNATATGCATCTCAGGGCCAGCACACAGAGACGAG
AGNGGCATANACTGTGTGAGANTTTTNGANACNGNCTTTCACANAAAGGGCNTATCTAAATGTGCNTG
TGCACACGCACANGGGAGAAAANCTTTNACNGGNCCNNTTNTGGGAAAAGATTTTGCACCAANANAG
AGTTGTGGAAAAACACACGCGCCCNCACNGGCGAGAAGACCTTTTTCATGTGAGATTTGTGAGAACGC
TTTTCANAAAAAGNGGGCCTTCTTCATGAAATGTTGTCCANCNCCCCCNCCAACAAGGNGGNGCCCCC
NCTTNTTTTAGNANAATGTTGGGAAGAAACTTTTTANATTGGTGGAGGAAACTTCTTCNTCCNCCTNAA
NGAAANAAAACAACGGGGGGGGANGNAAGGAACCNNCNGGCCCCNCAATTGATGGGTTACNAANNN
ANNCCCCCGCCNGGGGCCCCCCCTTCCTGGGGGGGGGGCNNGGANGANACATTCCCCCGGGCNGGGG
GGNAAAAAAAAACCNCCCNNNNGGGNNAANNNTTNTTTNAATTT
NCAAAANCTGGAGCTCCACCGCGGTGGCGGCCGCTCTAGAACTAGTGGATCCCCCGGGCTGCAGGAA
NTTCGGCACGAGGCCTGGAGCATGAAGCCCGGAACTGCATGAACCGCTCAAAACAGGTCTTCACGTGC
TCTGTGTGCCAGGAAACGTTCCGCCGNAGGATGGANNTGCGGNNGCNCNTGGNGTCCNANNCGGGGN
AAANNCCTTACAAGNNNTCNTCNTGTTCCNANNANTTANTNCAAAAAANGGACNNGCAAANCCNNNT
NATNAACTTGNNCGNACCCCCNANCCCCATNNCTNGNCCAACNNGNNCNANNGNNTTCTNGTCCNGA
NCGNACTTGNACTNGCCAAGGGTTNAANCACCNTGNGGGAGAAATGTTTGTGTGCCAAGAGATGCGC
GGCACACNGGATCTAGCCCCGNATGTGGNTTGNNATATGCATCTCAGGGCCAGCACACAGAGACGAG
AGNGGCATANACTGTGTGAGANTTTTNGANACNCNCTTTCACANAAAGGGCNTATCTAAATGTGCNTG
TGCACACGCACANGGGAGAAAANCTTTNACNGGNCCNNTTNTGGGAAAAGATTTTGCACCAANANAG
AGTTGTGGAAAAACACACGCGCCCNCACNGGCGAGAAGACCTTTTTCATGTGAGATTTGTGAGAACGC
TTTTCANAAAAAGNGGGCCTTCTTCATGAAATGTTGTCCANCNCCCCCNCCAACAAGGNGGNGCCCCC
NCTTNTTTTAGNANAATGTTGGGAAGAAAGTTTTTANATTGGTGGAGGAAACTTCTTCNTGCNCGTNAA
NGAAANAAAACAACGGGGGGGGANGNAAGGAACCNNCNGGCCCCNCAATTGATGGGTTACNAANNN
ANNCCCCCGCCNGGGGCCCCCCCTTCCTGGGGGGGGGGCNNGGANGANACATTCCCCCGGGCNGGGG
GGNAAAAAAAAACCNCCCNNNNGGGNNAANNNTTNTTTNAATTT
TTNANAAAAGCTGGAGCTCCACCGCGGTGGCGGCCGCTCTAGAACTAGTGGATCCCCCGGGCTGCAGG
TTACACCCCACCCCCCAACATAAGGACAAGTCAGTTTGTCAGTAGCTTCCTCTGGCTTGAACCCCCTCG
ATTTTTATAGCCCACTTAACCATGCATAACAGACAAGTCCCATATTTGTCAGTAGATGCCTTTTTTTTTT
TTNCCCCCCCNGGNTTAANCCTTAAGGGNCNAANCNNAAAAAAAAAAGNCGNANCCGNTTANNAAAC
CNACTTAGGGCCTTGGAATTNAACTTTGGCNCTGGGANTAACTTACCTTTTNAGNCCAAANGGCCCC
CGGGAGCCTCCCCAAANCCTTTGNCCCGGGGAGGGGGNGGGGGGGGGGGCATNTAAAAANAAGANN
TCCCTTCATTGGGAAAAAAATNGGCCTTTTNTTAATTTAAATTTNCCAAANATTTTCTTTTTCCCTTTN
CGNGGGNGCCCCCAACCCCCCTTTGGGAAAATTGTTGCCAATTGGGTTTTTGGGGAAACCTGNTTT
GANATTNAAAACCTTTTTTTTTTCCCACCCGAACCTTTGGGGAAANAAATTTGCCCGGGGTTCGGGGA
GGGNNCCAAGAAAGGAAAACTGCCAAAACCGGGNANAAAAATTGGGAAANAAAGGGGGACCTTTGG
GAANAAAAATTTTTTTTTTTTTTAAAGGGGGGGGGAANGGGGAAACTTAAANGGGCCTTTTAAAAAA
TTTGTTTTTGGGGGCAAAAANNCCCCCCCNTTTTTTTTTGGGGAACTTTTTTAAAAAANNNCCCCCCCN
AAANGGGNAGNAAAAGGNTTTAAAAAAAAAAAAACTTNTNGGGGGGGGGGGAACNNNAAAAGGGGGT
TTTTTTTTCCCCCANAAAAANATTTTTGGGGNAAAAAAAAATTTTTTTTGGGNAAAAAAANCNTTNAA
NGGGGGTTTGGGNNNNAAAAAAAACCCGGGGGGT
TTGGANACCACTGGACTCCACCGCGGTGGCGGCCGCTCTAGTGGCTAGTGGATCNCCCGGGCTGCAGGT
TAAGGAAGTCAGCACCTACATTAAGAAAATTGGCTACAACCCCGACACGGTAGCATTTGTGCCAATTT
CTGGCTGGAATGGTGACAACATGCTANAACCAAGTGCTAATATGCCATGGTTCAAGGGATGGAAAGTC
ACACGCNATGNCTGGCNTTNCNANNNGAANCNCNNTGCCTGAACCTNTNGATTGCTTTATGGCAGNAA
CTGGCCCNATNNACAAACCNTTNTGTTTGCNTCTCCAGGATTNCTATAAATTTGGTTGGNATNGGNACT
GNNCTTGTGGTTCTGNGTNTAGANATGGTGTTTTNAAANCTGGCATGGTNGGNCACCTTTGCTCCAA
NAACTGTANCANCTGNAGCTGAAAGNTTTTTANGAANTGCNCCATGAAAACANTGAGTGANCCCCTTG
GNTGTGGACTATGTNTNTCTTATANTGNGNAAAAAACGNGNNNTTTCAACANATGGNCCGAANTNNN
NANNNTTTGCTTNNTNATCNGAAAAAANTTATNCANCCCNNTNGNAACCTNGNTTGNCTTCNNNGNNT
TNAGCGGNTTATTTNTGAAACCATTNNTTGGGCNATTTTANNTGGNTGGTTNNNTTCCCGCCTAGNGGT
TGGGANTNGNCACTACNNGCTTNNCATTCNCNTTNNAATGTTNTGNNTTGAGNTTNAAGNATAAANAA
TTGANTCCNTTTTTNCTNNGNAAAAACAGANTTTGAAAGAAATGGGCCTTATNTTNCTNTNNAAANCT
NGNTNGACACCCNCCCTTTNTCTCTTNTNTTGNATCCTNGTGATNTTCCNACTTCNTCNNNGNGCATNA
NNTTTCNTTTNTCTTNTCNCNTCCCGTGNNNCCTCNNNTNNCTATNNTGACCTCCTCNATTGGAAACTA
CNCTCTTACNNTCTNTTATTAGCNCTATTATNAGTNTTGTGTCTANTNTCTANCGCGTTTTTGAGATTA
CTNGNAATTTCNTTNCNNACTNNTTGTCCT
CAAAGCTGGAGCTCCACCGCGGTGGCGGCCGCTCTAGAACTAGTGGATCCCCCGGGCTGCAGGAAA
AAAGTCAGAAGTAGCAGTGCCACCATGGGTTGATAGTAATGATGAAGAAACAATTCAACAACAAATC
TTGGCCCTGTCAGCTGACAAGAGGAACTTCCTTCGTGACCCTCCAGCCGGGGTGCAGTTTAACTTTGAC
TTTGATCAGATGTATCCCGTCGCACTGGTCATGCTCCAAGAGGATGAGCTCCTGAGCAGGATGAGATT
TGACCTTGTCCTAAACTTGTGAAGGAAGACGTGTTTTGGAGGAACTACTTCTACCGTGTCTCCCTAAT
TAAGCAGTCGGCCCAGCTCACAGCCCTGGCAGCCCAGCAGCAGGCTGCTGGGAAGGAGGAGAAGAAC
CACGGCAGACAGAATGAGTTGCCGCTGACAGAGGCAGTAGGGCCCAAAACACCACCTGTCGTAATCA
AATCTCAGCTTAAAAGTCAAGAGGACGAAGAGGAGATTTCTACCAGCCCATGTGTTTCTGAGTTTGTC
AGTGATGCCTTCGATTCCTGTAACTTAGATCAGGAGGATCTGAGGAAGGAAATGGAGCAGCTGGTGCT
GGACAAAAAAGAGGAGGGGACAGTCGCACTGGAAGAGGATTCTGTAGATTGGGAGAAAGAACTACA
ACAGGAACTTCAGGAATATGAAGTGGTGGCAGAATCAGAANAAACGAGATGAAAACTTGGGATAAGG
AAATAGAGGAAATGCTTGAAGGAGGAAAAATAAACTGTTTGTTTCCTGAAANAAAANAAANAACTGN
AACCTTGCCTNTAACTGGCATTTAAATTTNTANAGTTGANAACTCNCTGAAATCAAAAGGGGCCAANG
GAATTTACNTTTNTAAAATTCAAAATTTTTTTGTTTTTNAGCCTGNAACNTGCCTTTTTTTN
TTNNNNAAAGCTGGAGCTCCCCGCGGTGGCGGCCGCTCTAGAACTAGTGGATCCCCCGGGCTGCAGGA
ATTCGGCACGAGGCCGAGCGCCGCTCTGGCCGCACTGCGCTCGCCCTGAGCTCCGGGCTCCTGCTAAG
CCAGCGCCGCTGTCGCCTCCCTCCAGTCGCCATCATGATCATCTACCGGGACCTCATTAGCCATGACGA
GATGTTCTCCCGACATCTACAAGATCCGGGAGGTCGCGGACGGGCTGTGTCTGGAGGTGGAGGGGAA
GATGGTCAGTAGGACAGAGGGTAACATCGATGACTCGCTCATTGGTGGAAATGCCTCCGCTGAAGGCC
CCGAGGGCGAAGGTACCGAAAGCACAGTAATCACTGGTGTCGATATTGTCATGAACCATCACTTGCAG
GAAACCAGCTTCACAAAAGAANCCTACAAGAAGTACATCAAAGATTACNTGAAGTCAATCAAAGGGA
AACTTGAAGAACAGAGACCAGAAAGAGTAAAACCTTTTATGACAGGGGCTGCAGAACAAATCAAGCA
CATCCTTGGTAATTTCAAAAACTATCAGTTCTTTATTGGTGAAAACATGAATCCANATGGCATGGNTGC
TCTTGCTTGGACTACCGNGAGGATGGGGTAACCCCATATATGAATTTTNTTTAAGGATGGTTAAAAAA
TGGGAAAAATGTTAACCAAGTTGGCAGTTACTTTTGGATCAATCACCTGTCGGCATAACTGGGCTGG
GTTACTGGTTTTNATTCCCCCCAACACCCNGGACTTAAAACAGAANGGGAACTGGATGNCANTNTNCA
AGCCTNTTTANTTTGNTTTTGAAACGTTGATTTATTTTGGGAGCGGGGNGGCNTTTGGTTTTTTGANA
AAAAACGGGGTCATNGGANGGTTGGGCTTAAAAAAAAAAAACCCCTT
CCTATCTGGAGCTNCANCNCAGTGGCGGCCGCTCTAGAACTAGTGGATCCCCCGGGCTGCAGGAATTC
NGCACNAGGTGTANNCNGGCCNCCNTTCAGCNCATGTGCCNNCCGNNANTGGNCATCTCCTGACTCCA
TGATTGCATGCTGGCTATTACATGNNCCCTGTGAGGNAGTCACNACCTCTNACACNGCTAACAGACTN
GANGATGAGGCCNTNNGCCCACTTTACCTGANNTTGTGNTCNGNCNTNANGTAGNAACCACATGCCCA
ATGNNTCATAGCCCTGGCTANNAGAACANGNACGACCTGNGCCAANANAGGACCTTTNCTNACGTGA
TNTNAGGAGGGCAGGGNTTCGANACTTTCTATATNNTATAGCACTTGTTNNGTTGCCTCTGCTCACCTN
ACNGAATATNAAAANTNNATCNGTGNGAGNGAAAGTCNGCTCGNATNGNATTCNTTGATTGATCANG
TNATTTGGGCTNCATCCCTTGNAANTAANGAGCTGTGCTATCNTTGTNCNACTGTNTTTACANACNNA
ATANCNATCTGTTNNNCCGGNATGCCNGCATTATTTTTGAATNAAANCTNTNNTTNNCANNTNGNTGG
ATCCNGACTNCNNGCGNNCGAACCNNNTTGNANTNGCTNTAATGCGGGANTGTNNTAGCCAAAAANT
NCGGGNTTNANTCNTCTTTATTTCTTNNACCGCTTNGGANCCTNGTTGCCTCTATGGCTNTTTGNNTGN
CAACNNCAACNACATNACCTNTTTTNTTACNTNTCCGNGACNCCTTTNNNNAGATTNACCCTNGTNCT
CNGNNAANNNTAATTTCATNCNAATTTNTTCCCTTTGAAGCCNTTNTTTTGCNACCCCCTTTGGNANGG
CNNTTNGNTTNCNCCNNGAAAATGGNANGTCNNTCNGGACCCCCAAAATACTAAAAAAANTGGTGTG
NACTGTTTCTATCTAACTNNTACAGNATCCCTTTTTGAGANGCGNNNACCGGCAANNNNGNTCNCCNC
TTTTATGAACNNTGGATTNTTTGGCNNTTANTGGCT
NCCTATNNGGAGNNNNATNNCAGTGGCGGCCGCTCTAGAACTAGTGGATCCCCCGGGCTTGCAGGAA
TTTCGGNNCGAGGGCCGGGCCTCTCAGAAACATCACATATCACTNGCAAGAATAGCATTGCATACATG
GATCATGCCAGNGGNANTGTAAANAANGCCTTGAANCTGANGGGGTCAAANGAAGGTGAATTCAAGG
NTGANGGAANTANCAAATTCCCNTNCCCAGTTNTGGAGGATGGTNGCCCAAAACACACTGGGGAANG
GGGCAAAACAGNNTTCCAGTATCAAACNCCCAAGGCCGTCAAACTACCTATTGNAAATATTGCNCCCT
ATGATATCGGGGGCCTGANCAAAAATTNGGGGNGNACNTTGCCCTNTTTGNTTTTTATAANCCAAACT
NTATCCAAAGTCCCANAAAAGCTTCACACNCCATATGNCCCTTNTGCTCNAATCNTGGCCCCAGNAC
AAGTGACCAAGTNCCAGTTNTTTTTTCCCTGANATCGAGGGTCTCAAAGGCCAAGAGGGCTTCCCTGG
NGGCTGNCATCTGNTGACTCTGAACAGNGTGGAGAGATGGCTGTTAAAGATCTCACGCCANNCTGGCC
CCNTCTGGACGCCTNTCTGAAGAACCCCCACANGACATGGCGCGGTTCTGCGCGAANACCANGNCTTA
TGAATGTCCCGCNCCCCNGGNCGTGAANGANCCCCCCCACAAGAAANCTGTTGGANNGCGAAAAAAN
CCCTGNTGTNTTTTNNNATCCCCAAAATTNNTTTTTCCCCCCANAACCNCCGGTNGCNTNNGGGGGGG
NGGATTTNCTTCTTCCTAAANNNNCCCCTTGNTTTAAANACNANNCCNNGGCCCCGGTTTTTTTNNNCC
GANCNAANNNCCANGGGTTTGGGGTNNNAAAAAAAAAACCCCNNCCCNNNANGGGGNNGCTNGGGG
NCCCATTCTTTTTNNNCCCNNNNAAAAAGAAAAGGNTCCNCCT
GCCCTANCTGGAGCTNCACCGCGGTGGCGGCCGCTCTAGAACTAGTGGATCCCCCGGGCTGCAGGGTC
AACATGACTGACAATGATCTTATCAATATTCTTGACCCTTTTTATCATCTTTCAACTAAAAGTTTCAAA
ACACAACTTTTATCACAATCCAGAACTGACACCAACAAAAATATTAAAACNAAACNCCCCTTGGGAAA
CAAAATGAACGAAAATTTATTTACCTCTTTTATTACCCCTGNAATTTTAGGNCTCCCTNTCGTAACCCT
TATCGNACTATTCCCAAGCCTACTATTCCCAACATCAAACCGACTAGTAAGCAATCGCTTTGNAACCCT
CCAACAATGAATACTTCAACTTGGATCAAAAACAAATAATGAGTATCCACAATTCTAAAGGACAAACAT
GAACATTAATATTAATATCTCTGATCCTATTTATTGGATCAACAAACCTAGTAGGCCTATTACCCCATT
CATTCACACCAACAACACAACTATCAATAAACCTAGGCATAGCCATCCCCCTGTGAGCAGGAGCCGTA
ATTACAGGATTCCGCAATAAAACTAAAGCATCACTTGCCCATTTCTTACCACAAGGAACACCCACTCC
ACTAATCCCAATACTAGTAATTATTGAAACTATCAGCCTTTTTATTCAACCTATAGCCCTCGCCGTGCG
GNGGAACAGCTANCATCACTGCAGGACACCTATTATTCACCTAATCGGGAGGAGCTACACTTGCACT
AATAAGCCATTAGCACTACAACCAGCTCTTAATTACATTCACCATTCTAATCCTACTAACCAATTCTAG
AGGTTGCAGTAGCTTATATTCCAAGGCCTATGGATTCACCTCTTCTAAGTCAGGNCCTATTNTCTTGC
TTGGACAACCCCCTTTNNNNNAAANNNNNNNNNNNGNNNGC
NNNTATNTGGAGCTNCACCNCNGTGGCGGCCGCTCTAGAACTAGTGGATCCCCCGGGCTGCAGGNNC
NNATGACNATCNTGNTGTTNTNATNGTCTGANGCNNTNTCCGGGGTGNNNNNNTTTNTNTTACTTACN
NGTANCCNGNCCCCCCNTCCNGTGNNGGGTGGCCTNNCCCANAAATNGCCNATTTTTNTTNGGGNCCN
NAAAGGGNCANNNGGNATAAANGCCANNTNNNTGNCANCTTGGNAAAAAACAAGGTNNACCTTTNN
NCCTTGAANGCCTGGAANCCNGNNCCTTTTCCNNCCNCNNGGACNTTGNCAAAANNTGGNGAANACN
NGGNTNTTAAAATNGNAATGNACTGCNCCCCNTTNANNGTTAAAGGGNNGACNTAATTNTNNGTTGT
GTCTCNACTTTATACACNCCNCATTTATAGGCCGGGGCNCNTNCACAAAACCCCGGGNACAGGGNGN
GAGGGCCNCANATNNCCCGNGCNANANTCACACACTCCCCCCTTTGNATNNATGGGCANATGNTGTGT
TTCCTNTCCNGTGCGCNAGTGCATGTGCNCNCNTCTNTATATACCNTGGCTCNANNAGGTGGCNNTA
TNANAATTTATNACCGTTTTTGNATNNGACAATGTNGCAGNAANNTACTGCTGNTTATCAGACTGCTT
GTGGGGGCNNCAANAATCATNTTACTCTNGNGACCCCCCGANGAACAGGGGGGGGCCCCCTTCTCTTT
NATTNNTCTTNGGGAGGGCNCCCTTNTTNNTTTAANCCNCCCCCCTTCATTNNNANNAANTTNGNGNC
NNTAANAAAAAAAAAAAAAAANNGGGNGGGGGGGGGGGGGGGGGCCCCCTTCCNTCCCTTTTTTTCC
CNANTTTNTTTAATAGNTGGGNNNANAAGAANNTTTCNTNNGAANAAAAANACCCCNNANNGGNGAN
ANANACNNTTTTNTTTTTTC
NNNTATNTGGAGCTNCACCNCNGTGGCGGCCGCTCTAGAACTAGTGGATCCCCCGGGCTGGAGGNNC
NNATGACNATCNTGNTGTTNTNATNGTCTGANGCNNTNTCCGGGGTGNNNNNNTTTNTNTTACTTACN
NGTANCCNGNCCCCCCNTCCNGTGNNGGGTGGCCTNNCCCANAAATNGCCNATTTTTNTTNGGGNCCN
NAAAGGGNCANNNGGNATAAANGCCANNTNNNTGNCANCTTGGNAAAAAACAAGGTNNACCTTTNN
NCCTTGAANGCCTGGAANCCNGNNCCTTTTCCNNCCNCNNGGACNTTGNCAAAANNTGGNGAANACN
NGGNTNTTAAAATNGNAATGNACTGCNCCCCNTTNANNGTTAAAGGGNNGACNTAATTNTNNGTTGT
GTCTCNACTTTATACACNCCNCATTATAGGCCGGGGCNCNTNCACAAAACCCCGGGNACAGGGNGN
GAGGGCCNCANATNCCCGNGCNANANTCACACACTCCCCCCTTTGNATNNATGGGCANATGNTGTGT
TTCCTNTCCNNGTGCGCNAGTGCATGTGCNCNCNTCTNTATATACCNTGGCTCNANNAGGTGGCNNTA
TNANAATTTATNACCGTTTTTGNATNNGACAATGTNGCAGNAANNTACTGCTGNTTATCAGACTGCTT
GTGGGGGCNNCAANAATCATNTTACTCTNGNGACCCCCCGANGAACAGGGGGGGGCCCCCTTCTCTTT
NATTNNTCTTNGGGAGGGCNCCCTTNTTNNTTTAANCCNCCCCCCTTCATTNNNANNAANTTNGNGNC
NNTAANAAAAAAAAAAAAAANNGGGNGGGGGGGGGGGGGGGGGCCCCCTTCCNTCCCTTTTTTTCC
CNANTTTNTTTAATAGNTGGGNNNANAAGAANNTTTCNTNNGAANAAAAANACCCCNNANNGGNGAN
ANANACNNTTTTNTTTTTTC
TTGAANAACNNTCTGGAGCTCCACCGCGGTGGCGGCCGCTCTAGAACTAGTGGATCCCCCGGGCTGCA
GGAATTCGNCACGAGGCNTANTACAGGNTCGGTCTNCTTGCCATNNTCTTGGGTCTGCNTNANACCC
ATTAGGTTNANATAGANCACTTACACGNGNNTAACCTATGCCNCCNNNATNTTTNACANCNNNNCNTA
CTTCTNTNGNCCCCGCTNCCGNATAANCTTTTCGGCCTTTTCCCGAGTGGNCAGCACCTTGNANCTTTC
NNGNCGGCCTGNGCNCCGGCNTNANCNTGGATGGAAACNACGGTGGGGGCCCCCTGTTTGGGGGGCA
TTACAACTGTTTCCGTTAACCCAAGCCTGGTTGACCCCCNTTAAGCTGGANGGGGAATNCCNANATC
CANGGCCGNCCGNCCCCANGNNAAGGGANCCNATTAATGACCCCTTNNANAANAAAATTTNNCCTTC
TTTATTGGANAAAGGGGCCGGGTNCCTNGGNNCNNNCANAAACCAGGNTTTTGGGANACCNNAANTG
GAANCCTTTCTTNCANNNNCNANGAAAAACTNGGCCCGGAANNAANATTANAANNATNTTTNNTTTG
NNAANNTNCATTAAACAACCTTTCCNNTTGGGNGNTGGAAAACTTTTGGCCCNCGNAGAAAACNTTA
AAATTNGNAANTNNNAAACTTTGTGAAAAANTNNTNNGGGNCTTGNGGGGGGGACCTTCTANNNANC
CAANTTTTNGGGGTTNAAATCCCCAAANNCCCNCCNANACCNTTGGNAAAAAGGNNTTNTTNANAA
TNAAAAAAANGNNNNNNNANAAAACNTTCTNNNNAAACCGGGGAAACNTNNNNANNCCCCCCCTNN
NTAAAGGCCTCNCCCNNNNNNNNTNNNNNNTTTTAAAAAANGNTTTTTTTTTGTAAAAATNNNNNNNN
CNNNNNATCNCNNNTTTNNAANTTTTNTGACCNCCTCCCNNGGGGNTTCTCTTTCTTTGNCAAAANAN
CACCCNTCCNNNGGNCTTNNGANGGNGNCNTTTTCT
TTTNAACAANAGCTGGAGCTCCACCGCGGTGGCGGCCGCTCTAGAACTAGTGGATCCCCCGGGCTGCAG
GTTAAGGAAGTCAGCACCTATTTAAGAAAATTGGCTACAACCCCGACACAGTAGCATTTGTGCCAATT
TCTGGCTGGAATGGTGACAACATGCTAGAACCAAGTGCTAATATGCCATGGTTCAAGGGATGGAAAGT
CACCCGTAAGGACGGCAATGCCAGTGGAACCACCCTGCTTGAAGCTCTGGATTGCATTCTGCCACCAA
CTCGCCCAACTGACAAACCCTTGCGTTTGCCTCTCCAGGATGTCTATAAAATTGGTGGTATTGGTACTG
TCCCTGTGGGTCGTGTGGAGACTGGTGTTCTCAAACCTGGCATGGGTGGTCACCTTTGCTCCAGTCAAT
GTAACAACTGAAGTGAAGTCTGTAGAAATGCACCATGAAGCATTGAGTGAAGCCCTTCCTGGGGACA
ATGTGGCTTTAATGTCAAAAACGTGTCTGTCAAAGATGTCCGTCGTGGCAATGTGGCTGGTGACAGCA
AAAATGATCCACCCATGGAAGCTGCTGCTTCACAGCTCAAGTGATTATTTTGAACCATCCAGGCCAAA
TCAAGTGCTGGATATGCACCTGGGCTGGATTGTCACACAGCTTACATTTGCTTGCAAGTTTGCTTGAAC
TGAANGAAAAAAATTGGATCGTCGNTCTGGGGAAAAAACTGGNAANAAGGNCCCTAAAATTNTTGAA
ATCTGGNGACNCTTGCCATCGNTGGAAATGGGTNCCTGGCNAANCCCCTTGGNGGGCNAANAACTTTT
TTTTAANTATNCCTCCCCTTGGGNCCGGTTTTTGCTTGGGCCGGGAAATTGAAAAAAAAAAANNCNNT
TGGGGGGGNGGNAATNNAAANNCNNNNGGGACAAAAAAAGGCNANNTTGGAAACTTGGCNAAGGGC
CCCCAAANTTTTGCCCA
AAAAGCTGGAGCTCCACCGCGGTGGCGGCCGCTCTAGAACTAGTGGATCCCCCGGGCTGCAGGTTAAG
GAAGTCAGCACCTATATTAAGAAAATTGGCTACAACCCCGACACAGTAGCATTTGTGCCAATTTCTGG
CTGGAATGGTGACAACATGCTAGAACCAAGTGCTAATATGCCATGGTTCAAGGGATGGAAAGTCACCC
GTAAGGACGGCAATGCCAGTGGAACCACCCTGCTTGAAGCTCTGGATTGCATTCTGCCACCAACTCGC
CCAACTGACAAACCCTTGCGTTTGCCTCTCCAGGATGTCTATAAAATTGGTGGTATTGGTACTGTCCCT
GTGGGTCGTGTGGAGACTGGTGTTCTCAAACCTGGCATGGTGGTCACCTTTGCTCCAGTCAATGTAACA
ACTGAAGTGAAGTCTGTAGAAATGCACCATGAAGCATTGAGTGAAGCCCTTCCTGGGGACAATGTGGG
CTTTAATGTCAAAAACGTGTCTGTCAAAGATGTCCGTCGTGGCAATGTGGCTGGTGACAGCAAAAATG
ATCCACCCATGGAAGCTGCTGGCTTTCACAGCTCANGTGATTATTTTGAACCATCCAGGCCAAATCAG
NGCTGGATATGCACCTGTGCTGGATTGTCACACAGCTCACATTGCTTGCAAGTTTGCTGAACTGAAGG
ANAAAAATTGATCGTCGTTCTGGGAAAAAGCCTGGAANATGGNCCCTAAATTTNTTGAAAATTGGGGG
ACCCCTGCCATCGTTGGATATGGGTCCTGGGCAAAGCCCCATTGTGGGTCGNAAAGCNTTNTTTTGAA
TTATNCCCCCCCCTNGGGCCCGTTTTTGGTTGGGCGTGAAATGGAAAAANAAAAAANNTNCNTTTTN
GGGTGTCTTTAAAAACCGGGGGGAAAAAAAAAGGCANCTTTGGGACCTTGGNAAAGGNNCNCCCAAA
TTTTGNCCCAAAAAAAN
TTNANCAAAACTGGAGCTCCCCGCGGTGGCGGCCGCTCTAGAACTAGTGGATCCCCCGGGCTGCAGGA
ATTCGGCACGAGGCCGANGTGNNACGCGCGAGGCTGCTCTGCCGCCGCTGANATGCGTNACGTTGCCT
CCTACTTGTNGGCCGCCCTCNGGGGCNACTCCTCCCNCANNGCCAAGGACATNAAAAAGATCCTGGAC
AGTGTGGGCATCTAGGCAGACGACGATCGGCTCAACANGGNCATCANNGAGCTCCACGGAAAGAACA
TCNAGGGACGTCATTGCTCANGGTATCGGCAAGCTGGCCAGTGTGCCGGCTGGTGGGGCTGTGGCCGT
CTNCGNTGCCCCAGGATCGGCAGCACCCGCTGNGGGTNCTGNTCCAGCCGCAGCAGANGANAAGANN
GANGAGAANAAGGAANANCCGGAGGAGTGAGATGATNACANGGNCTTGGGCTNGTTTGACTANAGTC
CCGCTCCTCTGCACANAAAACCNTTTTTATGTAGGANACTTATTACANNANNNNNNTGGGGGGGGNCC
CAACCCNNCCATTACNNTATTGGNCNGTTTTANAAANNCCNNNTNANNCCNNNNNGTGGGNGNCCNNT
TNCCAANTCANNNCCNAANGGGGATGNNNTTNNCCCNNNNNNNGNNNNTTTTNNNNANGNGNNNNN
NNNNNNNGNNNNNNNGNNNNNNNNNNNNNNNNNNNTNNNNNNNNGGGGNNNNCCNNNNNNNNNNN
NNNNNNNTNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNN
NCAANANCTGGAGCTCCACCGCGGTGGCGGCCGCTCTAGAACTAGTGGATCCCCCGGGCTGCAGGAA
TTCGGCACGAGGGTCATGCAGGCCCTGCTGGGCCCGCAGTCCCTGCCTCGGACCAAGGAGGAGAAGC
ACAAGCCAGCCCCCCAGGAGGACTCGGTAGCTGCCAGCAAGCAAGCTTCCCCAGCCCCCAACATCAG
CAGCGGCTCCCAGACCCCCCGGCGGAAAAAGTCGCCGTGATCTGTCAGCCACATGTCCTTAGAGGGTC
AGGGTCGCGCTTGCCTCTGGCTCTCTGCCACAGGAGGGCCCAGCCGCCGTGCCTTAGCCAGCTGCCAT
GGAGACGCAGGTCAGGGTGCTGGCCTGTGGGGGGCGCCAGCCCTTCTGCTTCAAGGGCATCCCCCCAC
CCCCACCACGTCCGGTCCCAGGACACCCTCTGACCTGCTCGACGGCCCAGGAGGCCCATGTGTGACCT
GGCAATCGGGGACCCTTGCTCTCTGCCCCCACGGCAGGGGTCCGAGTCTGAGCCCCAACCCCGGCTCC
TGCACACGGGGTGGAGGTAGGGTGGGATGAGGCTGACCTGCTGGGGAAAAGGAGGGAAGACGCCCC
AAGGTCTCCTCTACAGCAGCCTGTCTGCGGTCCGGATCCGTGAACCAATAAAGGCAATTGTGTCTGAC
CAAAAAAAAAAAAAAAAAAAAAAAANTTGGGGGGGGGGGCCGGGNCCCAATTNCCCCTTTANNNGG
GNGNNNNNNNNNANNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
C
NNNANCTGGAGCTCCACCGCGGTGGCGGCCGCTCTAGAACTAGTGGATCCCCCGGGCTGGCAGGGAA
AAGGTNGAAGATCTCCAGCCCGAATTCATAGAGATTGGTAATCAACCANAGGTTCACGAGCAGACATT
TGACTGCTTACCTATCCAGACATTCTCTGGTCTCCCTAAATCTGAACCCACACCATGGATGATTTTGAA
CGTCGCANAGAACTCAGAAGGCAAAAGCGAGAAGAGATGCGTCTGGAAGCAGAAAGAATCGCCTACC
ANCGGAATGACGATGACGAGGAAGAGGCCGCCCGGGAACGGCGCCGCCGCGCCCGACAGGAAAGGC
TGCGGCAGAAGCAANAGGAAGAAGCCTTGGGGCAGGTGACCGACCAGGTGGAGGTCAACACCCANA
ACAGTGTGCCTGACGAGGAGGTCAAGACAACCACGACCAACACTCAGGCGGAGGGCGATGATGAAGC
TTGCTCTNCTGGANCNACTGGCTCGGCGGGAGGAAAGACGCCAAAAGCGCCTCCAGGAACCCTGGAC
GACAGAAGGAGTTTGACCCACAATAACAGATGCCAGTTTGTCNCTCTNCACANAAGAATGCCAAACC
ACNCCACAGACAATGAAACNGCGGAAAAGGAAGGAANAANTGAAAGTCCCNGGAAAAGGCAGGACC
TGGAAGNAAACCGANATACTCCCCCNGTCNCCCCACAAAAATGGATTGGGATGGAACCCGGNANAAA
AAANNNNNNNNCCGNNNNTCTTTTTNNNCNNNNNANNNNNNNNNCTTGGGGGGGGGGGGCCCNNAC
CCCNNNTTCNTTTTTTTTTNNNGGGGNNNNNNNNNNNNANNNNNNNNNNNNNNNNNNNNNNNNNNG
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNC
NNCCCCCGGCNCNTAGTCNTTNTCACTCNNNNNNACCNCNNTTNNAGCCCCTATAGTGGAGGTTCGTA
TTNCNTGGGCAGGNCGCTCTAGAACTAGTGGNTCCCCCGGGCTGCAGGCNTTTCTCNNCCCANGNTTT
TTTNTTTTTTTCCTNCTNTGCTCTCCNNGNCNNTTACCTCNGGGGGCCTNCCCGCTCNCNCNTTCTTTGT
TCCAGACTTTNTNGCTACGCNTCNGNCNGNTCNTNNNNCTCGCNNGCCTGCCCNCTCCCCGTNNCCTTGN
TTNTNATACTNNTTNTANTTGTTCNCCNCNCGCCGNCTTCCTCGCTCTTCTNTACTTTCNTTNACGACCN
NCCCTCCNGNTCTNANTCGTNCTTNNGCNNNGNNNGCNCTCTNGNNTTTTCTNCTNCNTTCNCGTGTNG
TNCCCNATCCNTCCTGGGCTCTTNNTCTNGCCGNCTCCGCAGNNNACCNNTNNANNCTNCNNTTTNCC
CGGGCCCNCCCNNCNGTCTCTCTTCNNAANNTTCNGCGGGNCTCGNNTNGNGGTCGATNNGCCTCNNN
NTCTNCGNTGTCNGCTCTCNNCGTTNTGNGNCCTNCCNTNNGCCGCCCNTGCNNCCTNCCNNTGCCAC
CNCNTNGACNNNCCNCTGNNNNTNTCCTNCNTNCGGTNNCTANGTNTTNNCCCTATCTNCGGGNNNT
TNNATTNTNNCTNGCNCTCNNNNGNNNGCTCCNANNCTGCGTGGNTCNCGNCGTNTTCCCCGNATTTT
GNCNGNNTNTNCCCCNCTNTANGGNTNNTCGGGCCTNCCACTNATTTTCTNNTCNNNTNGTNNCATNN
CNTNTCCCCCCCTACNNCNNACTNNTNGCCCNTCCCTCCGGGTNCCTCTTNNAAANNCTNCTACNTTNC
NCCTCTCTTNCCTCCTTCCNNCTCCCCCTCTACNTCTCCCGANTTAGTTTATNCCCTCNTCNGACTNTTC
TGCTCNNNNNCTTGNNCATNCTTTGNTTNTTCTGACTGACANGCCCGNAGCTTTGTTTCATTTNNGCCT
NTATNATTCNNCTCGTGNNNTCANCCGNCCTNTCTCATCTGCTNNTNCGCCTANCTCGTGNCTATNCNT
CCCACTCNANCCCCTACTCTCCNCTNCGTTATCCCCGNCNTAGTCCCGNACTTATTGNCTTCNTTTCCT
NTCNCCTNNTCANCGTTNCNNATCNCTTNCTTCTNTNTNACNNCAGTCTCTNCTACTGTCTCNTNCCT
CAGNTCCTNTNCATTCNTCGNNGTNTCCTCTTCNCCTTTGNNTACATCCTTNTGCNCCTNCCGNGCNCA
TNGCCTCNTTCTNTANTNNTCCTGCTGACTATANCCCNCCCCG
GCCTATANNGAGNNGNATTACAGTGGCGGCCGCTCTAGAACTAGTGGATCCCCCGGGCTGNAGGAAN
TTCGGCACGAGGGTCAGAGCCAGGTCCATTTTTTAAAAGGNAGCTGGCNGCTGTAATNACAAANCNTT
TTNANGGAAANGGNACCTAAAANTNNCTTNNNCCTTGNTNGTGNNTTTTNTTNAAAACNTTNACCNTT
NAAGGGGNAANNNANGGNNNNANAANGGGNCNTTNNCNGTTGGGNNNATAACNNNACATAANTGAA
CCCTNGNGCNCNNGANCCAATGNAACNGGGNGCCANCCNCCTNANGACACCGGAAGAAGCNTNAACT
NGAGACACCCGGAGAGAACCCTTACCTGCGCAGGGACNTATAGATNTGTNTGCACAGANATTTATAN
AAGAGANCTNAGAGGGGGANATAANTAAAANGCGTGGCTCNCATGCNGNGCTNTAAGATNGGGACTC
NCNATCTGATNCCNCAGGGAGCTCTATATNGCNNCCACTNTNCACGTGTGNTNNNCAANCCTCTATAT
GTNCCCNTANCCNCGCGCCTCNTNATCAATNCACACAATGGCATTTTTGTGGGGGCAAATTGTTNTTTA
NAAATAANNTCTCCACACCCCACCCCCATATATTAGGTGGAATAATAATCTCNAGGTGGTGCCCCTTTT
TCCCCTNGTNGCCCCCCANANAAGGAGGAAGATANNATAATTGGGNCTNGGAATNANCCATCCCCCT
GAACNTCCTGGTTTTTCCNCNAAANAACTACCCACTTGGNGGAANGTTGGCAAGCCTTCCCCTTNNNT
TNCTTGCTGGTNNAACCCTGGCCGTTGCCCNNCCAACCTTATTCTTCCTCCCCCCGAAAGAAACTTGAN
TTGANCCTTTGTTTGNGNGGGCCNGGGGGNCNGNAATAAAAAACCCAACCCNTTTCTTNTTTTTCCAC
CCNNCCCCCTTNAANGGGAAAAAGGNGCNTCCNTCC
GCCTATANNGAGNNGNATTACAGTGGCGGCCGCTCTAGAACTAGTGGATCCCCCGGGCTGNAGGAAN
TTCGGCACGAGGGTCAGAGCCAGGTCCATTTTTTAAAAGGNAGCTGGCNGCTGTAATNACAAANCNTT
TTNANGGAAANGGNACCTAAAANTNNCTTNNNCCTTGNTNGTGNNTTTTNTTNAAAACNTTNACCNTT
NAAGGGGNAANNNANGGNNNNANAANGGGNCNTTNNCNGTTGGGNNNATAACNNNACATAANTGAA
CCCTNGNGCNCNNGANCCAATGNAACNGGGNGCCANCCNCCTNANGACACCGGAAGAAGCNTNAACT
NGAGACACCCGGAGAGAACCCTTACCTGGGCAGGGACNTATAGATNTGTNTGCAGAGANATTTATAN
AAGAGANCTNAGAGGGGGANATAANTAAAANGCGTGGCTCNCATGCNGNGCTNTAAGATNGGGACTC
NCNATCTGATNCCNCAGGGAGCTCTATATNGCNNCCACTNTNCACGTGTGNTNNNCAANCCTCTATAT
GTNCCCNTANCCNCGCGCCTCNTNATCAATNCACACAATGGCATTTTTGTGGGGGCAAATTGTTNTTTA
NAAATAANNTCTCCACACCCCACCCCCATATATTAGGTGGAATAATAATCTCNAGGTGGTGCCCCTTTT
TCCCCTNGTNGCCCCCCANANAAGGAGGAAGATANNATAATTGGGNCTNGGAATNANCCATCCCCCT
GAACNTCGTGGTTTTTCGNCNAAANAACTACCCACTTGGNGGAANGTTGGCAAGCCTTCCCCTTNNNT
TNCTTGCTGGTNNAACCCTGGCCGTTGCCCNNCCAACCTTATTCTTCCTCCCCCCCAAAGAAACTTGAN
TTGANCCTTTGTTTGNGNGGGCCNGGGGGNCNGNAATAAAAAACCCAACCCNTTTCTTNTTTTTCCAC
CCNNCCCCCTTNAANGGGAAAAAGGNGCNTCCNTCC
TTNANAACAGCTGGAGCTCCACCGCGGTGGCGGCCGCTCTAGAACTAGTGGATCCCCCGGGCTGCAGG
AATNNNNCACNANGANNTGGNCCTGANNCCANCNCGCCANNGGNNNTTANGGGGTNGGCCCCAAACT
NGGNGTGNCAANATTTTCTTAATCTGANCTTCAAANNCCTACTGGGTGGAAGCCCTAANGCCGATATG
ACAAGGTGCCTTCCCCTATCTGGAACTTGGACTNTAACCTNCATACCGCNTTTGTNTNTNCNCGGACCC
TGNTGGNNGACANCTCNACTGACACGNTGGGACNCTGATGGTGATNGCCTGACTCCCAAATCNCCTNC
CCTTCNCTTATGNAAAGTGGTGGNAACAAAAAANCAANTTGCCNGGNNGGGGNGAGGAAACCACAAN
GNCCTGAACNAAGGAANGGTTCCTTGNCAAGGGNTGGCNNAAGGCCGNTTTTGNACNTTCTANTAGN
NNTNACTNNTTANTTCACCNGGTCACTTANTGGATTAATTAAAGGCCTTTATNNNNCTNGNAACCNT
NCGGANTTCATTCATTTCANGNACNGTTTAATTTGGNTAANCNTATANGCATGGGGAGGCNNGAAATA
GGGGACNCTNGGGTTNANANAAAAAGAANAAAACCCAACCCGCCGTGCCCCCCCCTTTTTTNTGAATC
NAAATTNTTCAAGGGGGAATGGNGGGNGAAAAAACNTTTTANTNGNGCCCGGCNNACTTTNAAATGG
NNAAANGGGGGGAACCTTTAANACCCCCCCGGCCCCTGGGGGAAANCCCCTTTTAAATCTTTTCCCCN
GTTGGGGATNCCANAGGCTTTTTTGGGGCTNTTTGGGGGGGGAAAAAGAAAAAAAATTTNCCCGGGN
CCCTTNCCNNTTCCNTTTGCCCGTTTTTAAAAAANGGGGCCNTNGGGCCCTTTGTTAAAAAAATTTCCC
CCATTNCNTTTTTNCCCCCTAAANCCCATTTGNGNCCCACCCCCTTGNGN
TTNANCACAACTGGAGCTCCACCGCGGTGGCGGCCGCTCTAGAACTAGTGGATCCCCCGGGCTGCAGG
NTCTNAACCAACCATAAAGGATTGGAACCCTTNATCTACTATTNGGNGCTTGGGCCGGTATAGNANGA
ACAGNTCTNNGCCTTCTAANTCGCGCTGAANNAGGCCAACCCGGAACTGGTGCTCGNAGACGACCAA
ATCTACAACGTAGATGTGAACCGCACACGCATTTGCAATAATCNTCTTTCATAGTAATACCAATCATAA
TTGGAGGATTCGGGTAACTGACTTGTTCCGCTAATAATTGGNGCTCCCGATNTAGCATTTCCNCGAATA
AATAATATAAGCTTCTGACTCCTCCCTNCNTCATTCCTACTACTCNTCNCATCCTCTATAGNNGAAGCT
GGGGCAGGAACAGGCTGAACCGTGTACCCTCCCTTAGCAGGCAACCTANCCCATGCAGGAGCTTANA
GTAGANNTAACCATTTTCTCTTTACACTTAGCAGGAGTTTCCTCAATTNTANGAGCCATCAACTTCATN
ACAACAATTATNAACATAAAGCCCCCCGNAATGTCACAATACCATAGCCCTCTGGTCGTATGATCCGA
ATAATACCGGNGGGCTACTACTACTCTCGCTNCCTGNATTAAGAAGCCGGGATCACAATGCTATTAAC
AGACCGGAACCTAAATACAACCTTTTTTGAACCCCGGGNGGAGGGNGGANANCCTTTTTNTATATCAA
CCCTTTATTCTGGATTNTTTGGGACNCCCCCCGAAAGNCTAAAATTTAAANCCTTACCCNGGGGTTNGG
AAAAAATCTNNTCAAAATCGGGGANCCTACTTCCTTCGGGNAAAAAAAANAANCCCTTTCNGGAATTT
TTNGGGNAANAGGGTNGGGGCTTTTAAAGGGCCAAACNGGAANTTNTAAGGGTTTCAATCGGGANGA
ANCCCCCCCCATTN
TNNNNNANAAGCTGGAGCTCCACCGCGGTGGCGGCCGCTCTAGAACTAGTGGATCCCCCGGGCTGCA
GGCGAGNTTTTTTTTTTTTTTTTNAANANANNCTANTTNTNTTTNNNAAANAAANGGGGGNGGGGGGG
NNCNNATGCCCTGACNNANAAGNAANCTCAACNNGNNTNNACCGNATNTTTTNTTNGCTNCAACNNC
TNTCCAANATGTTNGAGNGNGGANGGCANAAACCNTGATTTTTACTTGNCTCCNACGAAACCANCNTT
NTTTNNGNNCGGNNNAANTNCCNAGNNTTTNCCTTTTCCNNATNCANGNGGGGGNANANCCNAAAAN
TTTTTTCANAAANGGGTNTTTTTTNNNCNCCTTNANGGNNACCCCNNATGNGGNNNAAANGGNAAAN
NGCAANAGGGGANCCCTTTNNTTTGGTANNTAANGGGGGGNNGGNCCNNANAAAANTTTCNGGTTTT
NNNNTTTTGGAAAATAAAGGGNTTNCCCCCAAAACCTTTAAAGGGGGTTTNATGGGAAAATTAAAAA
AAAAAAAAAACCCCCCNCCTNCNNNGGAAAANAAATTAAAAGTTNGGGACCAAANAAGTNTTAAAA
AAACCTTTTTTAAAAAANNCCGGAATTTTTTTNTCCGGGAAAGGAAAACCTTTTTTTTTTTTTTTTTAAG
GGGAAAAANAATTCCNTNNGGGCCAAAAAANTTTTTTTGGGANGGGGGGGGGGGGGTTTGTTTTTCNC
CNAAAAAAGTTTTTCCCCNCCCNCCCCCCGGGACCCNGTTTTTTTNAAAACCTTNNGGGGACCCCTTTT
TTTAAATTTTCTTTGGGAAAAAGNCCCGGGGNAAAAAACTGTTTAAACCNGGGGGGNAAATTCNTTTG
GGGGGAAAAAAAATGNGGNTTTTTAACCCNTTTNAAAAAANGGGGNGGGGCCCAAAAAAAAAAAAA
ANGGGGGNGGGGAAACCNTTTCCCGGGGNCCCCTTGGGNG
TNNNNACANCTGGAGCTCCCCGCGGTGGCGGCCGCTCTAGAACTAGTGGATCCCCCGGGCTGCAGGN
NGGAGCANAGGGNCCCACCGGCGACTTCAGAGGGAACTCATTTACTGTTACCGTGCTCATCTCAGGAA
CAAAACCATTTCTTAGTGAGATAATATAGTATCAAAGCAACATGCAACCAAAACCTTTTGACATTTTA
GGGCTAGTTGGTACTTTCGAGTAGACATGTTTGGTGGCTGGTGTCAAAAGTCCGTAAAGCATTTGCTGC
CAAGTTAGTGGAATGCTTGCTTTTATTAAAATGACTGTAATCTTCCTTGTTACAGACAAGCTATCATTG
GAAACCTCCTTTTACAGTACAGGGTGCTCAGAAACGTTTTGAGTACTTTGAAAGCACTATATTTATGTT
GCTCCAAGGAGTTGATTTTTCTCCAGGTTTAAAGAGAATCACAGCTTCGAAACTTTCATTAAAAATGA
GAGACAGAAGCCACAGGGAAAATAAAGCAAATAGGATTTTCAATATAAATATCAGNGAGGAAAAAAT
ATCCCAGTCTGTTGGAGTTAGTGTTCACGCAGTCAAGAACGTCGNTTNCATTTGCTCANAAAATCCTTT
CATGGGAAGTTTGGAGAACCATGAATGTCGCAGACTTGGTCTGGTGGGGTTGCACTTTATTATGGGGG
GGGGGGGGGGNGGTTGGGGGGNTTAAAATAAAACCAAGTCATGNGCTTTTATTTCTACCTTGTAATTT
ACCGGANGTTCCCCCAANCTTTTTAANGGNGGGNCTTNNNNCTGGAAAGGTTTTTTTGGNNAAANGGG
AAGGGGAACCTTCAAAANGGCCGGAACCTTTTTTTTTTTTTTCTNAAAGNTTAANTNGAAAAAAGGGG
GCNTTTTTTTNGGAAANAAACCNGGGGGGCCCCCAAAAAAANGGGGGNATTTTTGGNNCA
AGCCCNCATAGGNGGAGNCGCATNACAGGGGGGGGGGCCCGTTNTAGTAACTAGGTTGGATGCCCCG
GGCTGCCAGGNANATNTNCNCGCANNAGGCNANTNNTCACCNCGTTTGNTCTAGGAAGTCTNNCGNA
TGAATNATGGTGCTCNTCNGCCAGGTGCAGCNTTTCGANCAGTAGNNTCTACCNNATGCCTTCATTTG
CCNATNANGGTTCACTGTNTAATNGCATGCATGAGCCACTNCGNANATATTGANTGTAGCGTNCGNNC
TGNCTTGGGACCCGGGAGTANTATTTCNGTNNCCTAGNGCGCANGNACNCCTNTTNCNTTCGTCNNAA
CNCCTTGTNACANGTCACGNCTATCANNAGACNCGNAGNTAAGNNANANNNGACNGTGCATNNNCTT
GTTTCACTCNCTANCNCTNTGANNTGNNTNGACNNTTNNAGNCTCTACACGNACTCCGNNATATNATN
TCNTNTAGNGGGCAACNNCCNTANTGANCANGGTCTCNCAATGGATGCGNACGNNCCCTNCNTCCNN
TTAACTNTTTNCATNTAAACCNTNTGGTGCCTTAANTANTNTNACTTNCATANCCACTCGCNANGTGCG
GNTGNTNACNTTNGGTNGTCACATTNNGACNNNAANNGTTCNCNGNANTNGCGACATNCTNNGACCC
ANNTGTAAANNATCNNNNTAGATCTCGGTCATNCNTCTTCANATNNANCNNGCGTGNATNGAGCNNN
ANNACGACATCCATTAATNTNCNTNGAANNNCGGGTTCNGNNACCNTANNATTTGNTNTCAACGNNT
NACTCAANNNCCNANNGTGAATTNTNTNTATCCCCCNTNNGNCGNCGCNGCGNCCCNCNANNTANTC
NNTNTGTCTNATGCCNCAAANTNGNNNTTAAANCNNTNTACTNNATCATTTTATNNCCTTCAANCNNG
TCGAACGCANNACAATANTCTACTTANANTTNCNNCNNNGTGTCGGNNATCAANNAANTGTCNGCAT
NGNTTNNNGTGNNTNNAACTNGAGNNCAAGACATCCTCTTCTNGCCGCNANCNTGCATCNGTGNGAT
NCANGCACTNNCNGTGNNNCTNCNNGTTCTNTANCATNNTCCTTCNATNNNNNNCAGANCNTCANACC
AAATNTANCGCTTCTCANTNTTNNATCCTGTACNNTNGANNCCNCGNNTNAGNTTNTTCTCNTANANT
TACGNTNTGTTNTGTTGGCGCANCNCTNCGAGTCNTATNATANGNTANNNNANAGANACANTTACNTC
TTACNANAGTNTGNGNCGNCNGNNGCCTAACTTCTTCNCACNCACGTNCANNATTTNTCNCGCNATCG
TNGTGANGACATGNNATGAGGNNTATTNANNCNCNTNAGACNNCNAGNTTATCTACNNTATNANANC
TACTNTNTNTCTCTNTCNTATNCCCC
NCAAANCTGGACTCCCCGCGGGGGCGGCCGCTCTAGAACTAGTGGATCCCCCGGGCTTGCAGGAATTT
CGGCNCGAGGCGGAGGCCGNACGATGCCTGGNGTTACTGNAAAAGATGTCAACCANCAGGANTTCNT
CNNAGCTCTGGCAGCCTTCCTCAAAAAGTCCGGNAAGCTNANAGTCCCTGAATGGNTGGACACCGTCA
NGCTGGGCAAGCATAAAGAACTTGCTCCCTACGATGAGAACTGGTTNTACACACGAGCTGTTTCCANG
GTCGGCACCTGTACCTCCGGGGTGGCGCTGGGGTTGGCTGCATGACCAAGATCTATGGGGGGCGCCAA
AGGAACGGTGTCATGCCCAGCCACTTCAGACAGAGGCTCCAAGAGCCGTGGCCCGGCGGGTCCTCCA
AGCCCTGGAGGGGCTGAAAATGGTGGAAAAGGACCAAGATGGGGGCCGCANACTGACGCCTGAGGG
ACAGANANATCTGGACAGAATTTGTGGACANGTGGCAGCTGCCAACAATGAAGCNTTAGACAAAATG
ATGCTGGGTTAATAAATTGCCTCGTTCGTAAAAAAAAAAAAAAAAAAAATTTNGGGGNGGGCCCGNC
CCCAATTCCCCCTATNGGGAGNNNNNNTANNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNC
GCCCTATAGNGAGTCGTATTACAGGGGGCGGCCGCTCTAGAACTAGTGGATCCCCCGGGCTGCAGGCT
GGTTACANCATNNAGTACNANAAGGCTGGGTCCCCTNCTAAAGANGTGGGNCCTNGGCCNCNNCCTG
GTGTCACATAAGCTACTATNACTGGGCCGGAACCNNGANCANATTAGNCANTCCAAGACATTGNCCTT
GANANCANTNAGANAAATGNGNCTTTGATNGGGANGAANANGACNGATGAACNNCNNNAGGTGGTA
ACCCTTGCACACCCCAATCTTCATGGACCAGCAGATCTTGGATGTNNCCTCCNCAGGACNAANGACCC
NTTTCATCANCNACCCTGNNNNTGACNCNGGAAACAGNATTNANNTTTCCNTTNCTTGTGGACNGCNN
NCCNNNCTTGGTCNANAAATGATCTTTGNNGANCATGGATTTAGNCCANCCACNNCGACCACCANGG
CCNCCCCAGTNAGGNNTAGGCGAAAACCGTNTCCGCCGAANGNAANNGATGANATCCAAATTGNCCA
TNTCCCCAGNGGAGACNTAGNCCNTCTTCTTTACCCTCACGTTGTGNGACTCNCTTCCTANTGCTTTCT
ACAGGGACAAGNAGNATTTCTCTCACACAACCNNTCTNATGNACCCGCATTCCACGAANAGCCTCTGA
GCNTATCATTGTCATGTTCATCCCNANTTAGGCTACTGATGAAATAACCCCTTTNCTGGTTTCCNAAAT
TCCCTTGGAACNCCNCNNGCTATGTGGCTANNCTNTNNACGGNGGNCTAACCNAGNAGGGGGCCCAN
CCCTNCCTANNTTTNNTTANGANGGANGGCNATGTATTANTNACCCACCTTCATTGGCTTGANTGGTTT
NNCCGAAGNAAGTTGGGTTTTNCCCGCCNGGNCNANNTCTTGNTAGACCCNGNGGGNCTANGNTTNN
NTCCCNCACTGANAGAACATNCNNGTNTTTGACCCCCCTACCGCCNNTC
GCCTATANNGAGNNGNATTACAGGNGGCGGCCCGGTCTAGAACTAGGTGGATCGCCCGGGCTGCAGG
AANTTCGGCACGAGGCTGACACTTTGTATTTCCTCTGGCGAAGAGAACTNGGCATCTTCCTNAAAACC
CTGCCCNNTCGGAAANTNNAAAACAAANCAANNCNCCNNTTTTTNACCCTTNANTTNCNANTTTTAAA
NTNAAANNNAANCCCCNAACCTTANTTTCNNGGNNNNCNNGCTNNNGGNTTTTAAGGNCCNTTANNC
CTTTTTTTNGGNCCTNNNCNNAACNTANCNAGAACTGGATGNCNNGACCTTGGTCCCANGTTAACNTC
ANNTCTGGNGTCATTANTTGCCCTGCCAGTGGTCTTTATAAAATTAGCAGGCNCACAATCTGGGAGGAN
ATATATTTAAGNGCANGGGGANANGCTCAATCATTGCGTGCGCTTTANNNGTATGANANANNNATCTT
ATCACCTNTAGGGANNAGAAAAGAGATGAGNGCGNTAATTTCANAAAACCTGAGTANGGAGGANATG
CGCCAGACAAGANCNTCGCTGNATTTCNCTCCCTNGTGGGNAGAGGNGNTACCCCTCTCNNCCAGAAC
CCCAGAAGAACCTCCACTTTTGNAGAAGCTCTTTTNTNCCNNAAGANCCTGGCTGAANAANACTGCCC
AAAACCATACTTCNAGAATGAGGANTCTCAGTGGGGGGGCNAAGNACTGNTGGGGGGCNCCATCGTC
ACAAGGNGGGTCNGGGAAGNTAATGGGGGNCCCTCCNTTTTTTNATTGGATGACCNCCTTAAACCNCN
TTTGGNAGGNANTTGGGAGGGGAACCCCNNCCCCAGCGGGGGGGAAANAAANACNCCGNTTNGTTCC
TTGGAANGNCTNNGANNNAAAAACANTTTGNCCCCNCGGGAAAGGANTNGAANGGGTTGGGAAAACC
TGGCCTTTCCNTTTCCCC
GCCTATANNGAGNNGNATTACAGGNGGCGGCCCGCTCTAGAACTAGGTGGATCCCCCGGGCTGCAGG
AANTTCGGCACGAGGCTGACACTTTGTATTTCCTCTGGCGAAGAGAACTNGGCATCTTCCTNAAAACC
CTGCCCNNTCGGAAANTNNAAAACAAANCAANNCNCCNNTTTTTNACCCTTNANTTNCNANTTTTAAA
NTNAAANNNAANCCCCNAACCTTANTTTCNNGGNNNNCNNGCTNNNGGNTTTTAAGGNCCNTTANNC
CTTTTTTTNGGNCCTNNNCNNAACNTANCNAGAACTGGATGNCNNGACCTTGGTCCCANGTTAACNTC
ANNTCTGGNGTCATTANTTGCCCTGCCAGTGGTCTTATAAAATTAGCAGGCNCACAATCTGGGAGGAN
ATATATTTAAGNGCANGGGGANANGCTCAATCATTGCGTGCGCTTTANNNGTATGANANANNNATCTT
ATCACCTNTAGGGANNAGAAAAGAGATGAGNGCGNTAATTTCANAAAACCTGAGTANGGAGGANATG
CGCCAGACAACANCNTCGCTGNATTTCNCTCCCTNGTGGGNAGAGGNGNTACCCCTCTCNNCCAGAAC
CCCAGAAGAACCTCCACTTTTGNAGAAGCTCTTTTNTNCCNNAAGANCCTGGCTGAANAANACTGCCC
AAAACCATACTTCNAGAATGAGGANTCTCAGTGGGGGGGCNAAGNACTGNTGGGGGGCNCCATCCTC
ACAAGGNGGGTCNGGGAAGNTAATGGGGGNCCCTCCNTTTTTTNATTGGATGACCNCCTTAAACCNCN
TTTGGNAGGNANTTGGGAGGGGAACCCCNNCCCCAGCGGGGGGGAAANAAANACNCCGNTTNGTTCC
TTGGAANGNCTNNGANNNAAAAACANTTGNCCCCNCGGGAAAGGANTNGAANGGGTTGGGAAAACC
TGGCCTTTCCNTTTCCCC
TTGAANACANCTGGAGCTCCACCGCGGTGGCGGCCGCTCTAGAACTAGTGGATCCCCCGGGCTGCAGG
CTANGTTGCNTNANCATGGCTAAACGCACGAAGANNGNNNGAATCNTGGGCNANTACGGGACCCGTN
ATGGTGCCTCNCTCATGAANATGGTNAAGANNATNGAAATCATCCAGCACGCCAAGTATACCTGCTCC
TTCTGTGGCNAAACCAAGATGAAAAGAATAGCTGTGGNCATTTGGCNCTGNGNTTCCTGCATGAAAAC
AGTANCTGNTGGTGCCTGGACCTACANCACCACCTCTGCCGTCACAGNCAAGNCGNNCATCANANGAC
TGAAGGAACTGAAGGACCAGNAGAGGCNCCACCATTCGATACCCTTGCTAGCCTATNAAATGGGTTA
ATTTATCTANCNGAANATCCTTTTTTCCACCTCGGGATGCCCGCNTNNTGTNAAAAGGGGTTCNCNNG
GNACCCTTTTCTTTAAANNTTAAAACCGCNNCGACCANCCTTTNGGGTAAAAANCANAGGAAAAAACC
NTNCAAACCGGNNTNTTGGACCNTAGACNANATCGNGCTTTTGGATTNNTGGGGAACANTTTCCGTGN
GCCATTTTGAGATTCAAATNTNGGTTTTTTGTTTGCAAANGCCANTTCNNNNCCCCTGGGCCTTGGGTN
GGGGNAAAANGGCCCCCCCNTTTGGCNAAAAANTTTCCTTGGCTTAACCTTTNAAAANGNTTNTTTTTT
TTTANCTNCAACGAAAAGNCCCCCTTGTTTTTAANNCCCCCTCNAANCTNGGGGNNTTTGNTTTGGNN
GNGTTTCCNANTNCNGNNNGAAAAAAACNCNNTTTANGGGGTTTTGNGGNGNTTNTTGGATTGTTTTC
TTGGGNNCCAAANAAANCTTTTNNAACCCAACNNNAAANGGGCCCCGGGGTGNNGCCCTTTTTTGGG
NAATNAATATTAANGGGTNT
NAAAAGCTGGAGCTCCACCGCGGGTGGCGGCCGCTCTAGAACTAGTGGATCCCCCGGGCTGCAGGGTT
TTGTTCCTTATTTTACTAATGATAATTAACATCCAACATGGCTTCTCATGCTATTTCTACCTCACTTTGG
TTTTGGGGTGTTCCTAATAATTGTGCACACCTGATTTGACAGCTTCACCACCTGTCCATTGTGTGAACA
TTTTCCTCCATTTTCTTTTTTCCTTTATAATTTCCAAAAGAAAACCCAAAGCTCTAAGGTAACAAATGAC
ATGAAGATTTGATTTTAGTCTTGGCTTTTTTTTTTTTTTTNCCNTTAAGGGNNCNNGGANTTTTTTTTTN
NCNGNAAATTNGGGCCTTTTNTTTTTTTTTTTTTTTTAANCCNGNNNTAAAAAAANGGGNTTNNTTTTN
TTNNNNAAAAAAANTTNAANTTTTTTTTTAAAANCNNGGGGAAAAAAANGGNNGGGTTTNGGTAAAA
NNTTTTTGGGTNAANTTTAANNTTTAAAANNNTNNNTNGGGGGGGGGCCCAAAAANTTTTNAAAAAAA
NCGGNANGCTTTNTNGNCNNNTTTNANNCNNNAATNGGGNNATTTTTTNGNGNNCNTTTANCCCCCCN
AGGNNNAAANGGGAAGCNNNAAGGGANCNNGAANCAAAACCCCCCCCGGGGGNGTTTTNNNTTTGTT
TTAAGGGGNCNGGGNTTTTAAATGGGGAACCNNAAAAAAATTNGGGTTAAACCCCCCCACCCNGGGA
CCCNTCCCCCAAANNTTTTTTTTAAAAANNTNNNNTTNNCCGGNGTTTCCCCAAAANATTNAAANNTT
TCCCCAAAAAAANNNCCCCCCNAANTGGGGNNAANAAANCCGGGGNTNNGGGGGGNNNCCNAAAAA
AANNCCCCCCCCTTTTTNTTNAAAAAAAAAAAAAAAAAAAAAANNNGGGGGGGGGGGGGGCCCCGC
CCCCTTNN
TTNNANCNCNATCTGGAGCTCCACCGCGGTGGCGGCCGCTCTAGAACTAGTGGATCCCCCGGGCTGCA
GGAATTCGGCACGAGGACGGNTTGCANCCAACATTTACAACAAACAGAGTTTCACCCAGAGTTTGAA
GAGGAGAATATCACTGAAGAATATATTTTATTCCTGTGGTGTAACCTATGAAATAGTATCCAACATAC
CAAAGGCAACAGAGGAGATTGAGGACCGGGAGACGCTGGCTCTCATGGCTGCAAGGAGTTGAGAATG
AAGGCACATCGGATGGGGAGACGTACATCGAGAAGTACACGCGGGGCGTGCTGCAGGTGGAGAACAT
TCTGAGCCTGGAACGACTCCGGCAGGCGCTGACGGCNAAAGNAGCACTTTCCACCAAAGCTCGGCAC
CTACCGCGAAGCCTCAGCACGCCCAACGTTCACAATGTCTNTTCCAGTCGACCAGACCTTTCTGGNTTT
GACGATGATGATAAGGGTTGGCCAGAGAACCAGTTAGACATGTNTGATTACAGCCCGAGTTACCAGG
ATGTATCGNGTTATGGAACTTTGCCTAGGGATTCACCTCGAAGGAGTAAAGAAGGTATGGTTGTACAT
TANAAAATNCTCNTGCCTTAANAGTCAGCCCTTTTAAAAGCATTTTTTTCTTAAGCCACCAAAGTTTTT
TTCAAGNCCNTTATTGCNTTGTAAAAAGAGGGGNCCTTTAAAAAAAGGGNNTAGNGGCTTGGAAACC
ANNGGCCCTTTTTTTAANCCCNGGNNGGGNCTNTTGGGGGGNGGGGGGGAAAATTGTTTTTNAAAGG
GTCTTAAACCGGGAAAGCTTNTATANTTTTNCCCNCCCCTTTNTGTTCCCTTGGGGGGAGGTTTCCGCT
TGACTTTNAAANNGNTTTNCCAAATGGCCCGGTTGGGAAAAAAACAAAAAAAANNGNGTTTTTTTTT
TTTAAAANGGNN
NCGNGCNCCNCNTGNCTCNTTNNNCNNTNNNNNNNNNTNNTNNNGNCNCCCCNCNCTTTNNTNNANC
GTCTANCCCTACNCCACNNNCNANCNCNCAAGGGGNNGTNCNATATTNNATTTGCNTATNTTTAAGCC
NANCNGGAGTTCGCATNACAGNGGCGNNCCGCTCTAAAACTNGTGNTATCCCCCGGGCTCGNAGGAA
TTGGGCCCCAGGCNATNNTNTTATTTCTGCNTTTTGTTTNANCTGTNCCGGGNCNTCNNNNNGTNATCC
NNCTTNCATAACTNANNTTNTNCAGNACCACTCCTNGNNNGTNNNNNATAANNNNNGTTCTAATNCT
TNTCTCCCGGNCTNTTTTCTGNNCTCACTTCATTAAANNTNAATACNTTNNGCCNCNTNNTNNNTACAC
CATTNTTNGCTATCANNNNAGCANNNTTNTNNNCNNACCTNTATCNTCNTNNTCNTATNNNNNCTNTT
CNCTTNNTTCTTAAGCAGNNNGTNNTANTNNTNTNCTTNNTNNNNNNNNGNTTNNGCNCCNTCCNTAN
TTGNAACTACCTCCCTCTNANAANTTNNCNANTNCATNTTTCTCNCNTNNTCTATTTTTANTTTCTATTN
NNGNTNTCCCCNCNCTTCCTCTTNATTNANNTTTTNCNCCCCCNNTNACNNCNNCTTCTTGTNGTNCCA
GNNGGTCCCNNNCANTGTNTTTTNCTAACNCGCTTNNNAGNNNNTGTNNNTCNTNTNNTNGCTNNCNN
ACTNNNCNNNNGNATNNNGGCNGCTATNNNNNTTCCNNNCACNCANCCNCTNTTTNCNTCGAGTCTTN
NANTTANCCGCTTCTNANTCNTCNATNTCTATNATTNTTCTNNTNACTGNTAAGNCNGCCGGTNTCANN
GNTNGANTCATTANNTNTTANCANNATCACGNNCTAGCNANTTTTNNANCNNCTANTTGTNTAGTANA
TNNCNNTNNTTCNCATGCGCTTCNTNCTNTTGCNCTATCTNTTNCNNNNGATCNCNACNNTCCATCTCN
TTTANTNGCTNATTNNCACTTANNTGTGANCATATCNTANTGACACNGTNGCGGTGTATNAGCTTCNTA
TNNNTANCNCNNNNGTNNANNNCNCCTGNNNCNTNCGTANNGNTATTNTNTCTTTCNCANNNCNCGTAT
NCCNCCTTCGTTCNCNNGNAGNGTGNTNNCNGATGCTGCNCACTGTTCTCNNNTACANNGNTCNANNT
NTCTTTTACTCNCNNNCNNANACNCANGCNNNNNNAGNNCGNTTNANNNCNCNTCATCNTACTNATN
NANACGANAATNCNCTGACCNNNCANAGANATGCCTNNACACCGANCCAGACANCGACTGTCNCACG
TNTTGCNCNANNTNTTGNNTACNAGTNANANACGCTGCATAGNNATTNNTTNANTNTNCTNCGAGTT
ANANATCTANTGACAGNNATCGCTCGNATNNTNNATGNAANATTNATNTGNCGANAGNTTNNTNNNA
NGCTCNATNATCATNTGATNTTACGCC
NNNNACTGGAGCTCCACCGCNGTGGCGGCCGCTCTAGAACTAGTGGATCCCCCGGGCTGCAGGCCGG
CCTTCAAGTGGTGGCTGACAGGCCTGGTAGTGGGGAAGGGTGTCCTGGGTTGTTAGAGGGTCTCCAGG
GTCTTCTGGTTGTCCTTCCCTCCCTTTTCCACCATGAGATCTATGCCACACAGGCCAGTGTCTGCAGAA
CCTTGCTTTCTAGGTTTGGTTTCTGANCTCCTGTCTGTACTTTTGCCTCCTCCAAGGTTTTTTCCCCTAAA
AATGCCCATTCTCATGCCTCCCCACTGTGGGTCTGCTTGCCTTTCTCTTGCAAGTTGGACAGTCCTCTGC
CTNTTGGCCAGACCCCATCCATTTGGACTGCCTAAGTCACTCGAGATCGACCCCCACCCACACGTACA
ACCCCAGTGGCCAGAGTAGGCAGGGAGAAAGTGAAGAGAATCCAGCTGCCTCTCGAGGGAAGTGAGA
TGTGTGAGAGCACTGTANCTCCCCACCTGTGGACTCTGGGCCAAGAAAGGGGTGTGANTATCTGGCGC
ANGGTGCCAACATTCTTGCAAGCAGCTGCTGAAATCTGCATCTTCCCAGTTGGCTGGTACTCCATANCC
CTTGGACTTCTGCCCCCCTTCTTCTNCCTGTTGGTCTGCCCTTCTCANAACAGANCCTTTCCCTTCACTC
CNTGGCCCCCNTGGGCTGACCAAAATGGTGNTTTTTTATGGGANCCCCTTGGCCCAANAACCCTGGGG
GGCAAGAAAANACTGGCATGAATGNAAAAACCGCCNTCTNGCTTTCTTGGGAGGCAGGCTTTTGNGG
GCAAAAGGAAAAAGGGTCCCGGCCTNGCCCTTTGGGNAANGCAATTNTTNTTGTTGNGGAAGGGGGG
CCCTTCGACCCCCCCCTTGNGTTGTNGGGNCACCCCCCCCCCNNGGTTTNTTTTTTTAAAAAGAAACCC
AGGGNTTNCCNCCCCCCCTTCCCNGGGNANAAGGGGAANCTCG
GCCCTATNNGGAGCNGCATNNCAGTGGCGGCCGCTCTAGAACTAGTGGATCCCCCGGGCTGCAGGAA
TNCGNCACGAGGNNTGTCTTTANCGTNCNCNTNGAGGGGGNNNCCNGGTTCTCNGNAGCCCCNANAN
NNACACCTNNTNGNAATCGGNNNTNTCCNACAAANCCTGNCTGNCNNATNGNNANATNTNGNCNNAA
NATCCNACNANACNANAGTTTCCCATTCNTTCCTTNNNGGANNNAAAGACANTNAAAANNGGAAGCA
GGGNGGGNANNAGGGNNACNGNCCCNAACCCTNGAAAANNGCCTTNNAACCGNGGAGNGAAATCCC
NTACCNTATTNAACNGCCNGCCNNCTCTGNCAAAAACCNAANNATATTNCCCTNGTTGGTCNGTNAA
CCCNCAATNAAANACNGNATANANCCGGATACANATNTACCCATNTNGNNCCACCAANGCCAATGGG
TGGCTNCCTTTNTTACCAAANCNNGNTTTCNNTGGCTCTNAANGGCAGTCCGGCTNACNTTTAAAGAT
AANGGAANGTGGATNNGNTNCNATNAACTNANANGANNTTCGNAANGNNACCTATGNANAANAAGA
NCCCCCANNTTTGNTCCTNNCCACCTNTGGGTTNGGTTNNNNTNANCNNTAAAGGNANACTNCNATTT
CCCTCCGGNGANTAANTCNCTTACAAAAAANGCNANCAANNTATTTGNGCTACTTTTTAAAANACCAG
ATNNTCAATTATTNAGCCCNTTCTTATNCCCTCTTNANTCCATANNNCNTTGGGGGCNCNGGNCTGAT
TTTTAAGGTCTTNTNNTAANNNAATTAANTNTNNNACCNCTAAGGNAGCCCCNCCCCNNCTNCNANAN
NGGGNAANTTCNNNNTTTNNTCCGCNGNGCGNNGNNCCTTTNGTCNCACTNNCNNCTTNCCATCTTAA
NCCNNTNNGNGNCTNCTNTNNNTTATCNGTANGAGGTCNNNGGAGNTANNNGNACCANCCCTCCNNT
TNNCNGANGNNTNNNCGNC
GCCTATANNGAGNNGNATTACAGGNGGCGGCCCGCTCTAGAACTAGGTGGATCCCCCGGGCTGCAGG
AANTTCGGCACGAGGGTGACACTTTGTATTTCCTCTGGCGAAGAGAACTNGGCATCTTCCTNAAAACC
CTGCCCNNTCGGAAANTNNAAAACAAANCAANNCNCCNNTTTTTNACCCTTNANTTNCNANTTTTAAA
NTNAAANNNAANCCCCNAACCTTANTTTCNNGGNNNNCNNGCTNNNGGNTTTTAAGGNCCNTTANNC
CTTTTTTTNGGNCCTNNNCNNAACNTANCNAGAACTGGATGNCNNGACCTTGGTCCCANGTTAACNTC
ANNTCTGGNGTCATTANTTGCCCTGCCAGTGGTCTTATAAAATTAGCAGGCNCACAATCTGGGAGGAN
ATATATTTAAGNGCANGGGGANANGCTCAATCATTGCGTGCGCTTTANNNGTATGANANANNNATCTT
ATCACCTNTAGGGANNAGAAAAGAGATGAGNGCGNTAATTTCANAAAACCTGAGTANGGAGGANATG
CGCCAGACAACANCNTCGCTGNATTTCNCTCCCTNGTGGGNAGAGGNGNTACCCCTCTCNNCCAGAAC
CCCAGAAGAACCTCCACTTTTGNAGAAGCTCTTTTNTNCCNNAAGANCCTGGCTGAANAANACTGCCC
AAAACCATACTTCNAGAATGAGGANTCTCAGTGGGGGGGCNAAGNACTGNTGGGGGGCNCCATCCTC
ACAAGGNGGGTCNGGGAAGNTAATGGGGGNCCCTCCNTTTTTTNATTGGATGACCNCCTTAAACCNCN
TTTGGNAGGNANTTGGGAGGGGAACCCCNNCCCCAGCGGGGGGGAAANAAANACNCCGNTTNGTTCC
TTGGAANGNCTNNGANNNAAAAACANTTGNCCCCNCGGGAAAGGANTNGAANGGGTTGGGAAAACC
TGGCCTTTCCNTTTCCCC
TNANCAANANCTGGAGCTCCACCGCGGTGGCGGCCGCTCTAGAACTAGTGGATCCCCCGGGCTGCAG
GCCCGCGGATCTGTCTCTTGCTTCAACAGTGCTTGGACGGAACAGACCCAGGGACGCCCNTTGCTCCA
NCCTCCGACCACCNTCNANCNTTTTTTCNAGCCNNANCNTTNGNAGNNAGCCNTTNANTNGNCNGGAA
NNNCNGGANCNANCCNCNNTTTTTAANNTCNTNATANTANCNNNCNANNNNNNACNTCCNAAATNNG
NAAAANTANTNTNNCNNNGGGGNNGGCNGCNGCNACCNGCNGNGGTAACANNGNANTGGGGGGCCN
TCTAACCTATACTTTTNTTTGGGTTNTATTTTCACACGCGNAACNATGGCCCTGTGAGAGGGTGNGGNA
CANTTTTTTCGANAATNGNCCAAGAGAAAAGCGAGAGGGCGCNGAGCGTCTTNNNANNGTGNAANAN
NAGNGGGGGGGGCGCNCTCTTTNTGNNNNNGCNCAANCCNTATCAAAAAGAGAGGGGNGAAAANCA
CGAGACTNTNNNGGCGCCCTTTTCNAGAAAAAAAACCNAGNANCACCCNGTGGANATNTGGGNGCTG
NGTCTTGNCCCGAGACACCCCACNTGTGACNTCTTTGGTGGAAAACCTCCTTCTATGANGAAGGGGAG
NTCCTCATCAAGAAGGGGGGCNATCTTNNAACCTCCTCCGGATGGTTGGTGCCCNCCATGGTGNGGN
NGAGGAANTTTTTNTTAANNGNNGCTCCCCNTCNANNACTAATAAGGTTCTTGNGAACAACCGGNCGG
TCANTTGAAAAACCCCAACTAAANGGGNGGCTGGAAAGACCCCCCTTTTNNGGAAATAAGGAAGGTT
TTTTTTAAAAAACCCGGGGGGGCNCCNTTTNNTCCNNGGNNGGNANNGNAAAGNAAAAAANNTTT
TTGANAANNNCTGGAGCTCCACCGCGGGGGCGGCCGCTCTAGAACTAGTGGATCCCCCGGGCTGCAG
GAANNNNCGANGAGGCTNNACNCTNANANGAGCTTTNNGTGTNCTCNANGTNNAACNCCGCNTANCT
NTGTGATTNGCCTNNACNTCNNTGNNCCTGGTTNTGCTNNNCCNACCNCCATCTANNCTNTCNANATC
CNNATNATGNNATANATGANAACCCGGACAAAGCNCAATTCGTGGGCCCCCTTACCCTAGCATGTCCA
GAACGGNTTTANGGANTGGANTACGNTTTTTTNNTTATAAACNGAANCCNATTCTTTACCGNTTTTTTT
TGAGCTTAGTACCNNTAANNCCTTGCCCCGGCCCATNAATTAGGGGANTNCTGNCNGGGGNGNGGCN
TTANCCCATTAANCCTTTTNGANGTNNAANNGNNAATCACNTCTGTCNNATGGGTTTCCGAAGTTTCT
ATTACTTGAGCCAATNTTANNACAANNNAGGGGACNNCGAAGGNNNATGNTTACATGGCCTTATTNA
TGGCCATCNCCTTTGGTNATTGTTNTTAGGGCCAATAANAGGCNTTAAAATACACTTNNAACACACTTT
TNNNNATANCTCNCCCGGGTTTTTCCGGATNTAANTTTTTTTTTTTGTGNAAAAGGGGNTTCCCTTNGC
TCTAAAGGAAAANAANTGGGGGNCACCCCTTTTCTAAAATGNGNGAGNTNTCTTTTCGCCCNCNTTAA
AAAATTTTTTTTTCNTTTTTNNTAANCCNCCCTTTTTCNTCTTTTAAACCCNTNTGGCGGNGAAANGTNA
TTTTTGGAAAANGGGGNNTCACACTTTTCCCNTTTAGAANAATATTTGTTGNGGGGGGNNCNTTTTTAA
ANANGANGNCNCCNCTTTTNGNCCCNNGGNTTNTNNGNANTTNGCCCCNNCCCATNNNCCCNNTTNN
ACTGGGGGGGGGGGGGGGNTTNCCCCNTNATTTNNGNAAANNAAATATGGGGCCT
TTGANCACAGCTGGAGCTCCACCGCGGTGGCGGCCGCTCTAGAACTAGTGGATCCCCCGGGCTGCAGG
AATTCGGCACGAGGCTTTANATAGTATTTTAAAAGGTAACTAATGCTTTTGACAGTATAAACAAAAAG
CAAGAATATAAGGCCTAGAAAAATTGTCCTTCGAAAATATCAATGATGTACCCTGGAATGTGAAGATG
TCCCCTTATAGTAAATTGATTTATTTGAATATTGTAATCACTCTTCAGACTTTGAATGAGTCATTACA
GTGTGTATCTGTATATCTGTAGAGAAAAATGACAACCGAAATGTAACTTTAAAATCAATAAATTTGGC
ACTGTCTGTTGCACCACTACTGTATCTTTTTGAAAGAATTAACAGTATTTTCTTATTGTAAAAGGTAATT
CTGATGATTTCCACATTCACCAGACATGTTTTTTTTTTCCTGTACTATTTGCATCCCCAAATATTTAAT
ACAAGTAGGTTTTGCACGGCTTGANCNTTCAGAGATTAACAGAATGACANCTCANTTTTAATTCTAAA
GCCATGAAATGTTGCTTTGANACTGAACAATCTGAATCTTATTTATAATGACNCCCAATATCCATGTTT
CCCTACTGGAAAANACCTGACAGTTCTTGCCTACACAGCAAGAACTTATTAAATCTTGGAAATNCCGG
GCCTNTTTTAAATTCTGGGGANGCCGGGAAACAAAATATTTTGAGGGGAAATTGGTNTAANACAAAG
GCNTTTGAAAAGGNTTTGCTGGCTTNAAACCTCCAACAGGTACATTNTTCGGAAAGGCAACGATTCCC
TGCGAAATAAATTTAATAAAGGTAAAAGCCCAACCCTTAAAAAAAAAAAAAAAAAAACCTTGGGGGG
GGGGGCCCCGGNNCCCCAATTTCCCCCTTTTAANGGGGGNNNC
AGNNNNNNNNNGGNNNNNTTNANAAAGCTGGAGCTCCACCGCGGTGGCGGCCGCTCTAGAACTAGTG
GATCCCCCGGGCTGCAGGAATTNGNTNCTNNNNTTNCTANTNCNTTNTATAANNAGGCACAACCTCTG
GNNCATTTNTCANGNNNAGNNTGTAANNCGTGNCTCNATAAAANGCNNANNGGANTAANANTANNGC
NGCCTNNCTNCGTGAANGCTTNCCATANCTATTNTNCATTACATTCCACATCANGATATCGAAACNAT
TCTACANNNTATNNACACTAGCATNACATATNTNTATTNGCTTTACTTNCTTATACNNGGAAACTGNN
GGTTTACTCTNTNAAATATTTATTGTANGTCACTNTTANCTATCNNACTCTTGANNGGTNTNNAACNAC
TTNACCAGAACTACCTATTGGGTAANGATNACATTACTGANGCCACTTANAACTTNAACCTGNAATGN
TNTCTTGCNCTAAAAGTGAACTGNCTAANTTANTGAAAANTACNNGTGNCCCCTACGCANTTGGACNA
ATAAAAACCNTNNTTNTCTTNTNCCCAAAAANATGNNTTGNCTTAATTTCCCACATAAANGTTNATTTT
CACCTCAAATANACTTNAAAATTTNTTTCTATANGTNNGGGNCNCCCCAATTNTAAACTTTNCCCGNCC
CCNTTAAANAAATTCTTNTTCNAGTTTTTTTANCCTTTTTNCCCNAANAATCATTGGNGGGGGGCNCCG
TGGNAACNCACTGGCGNTCTTNTTCCNAGCACTNANTTTNNTNGCCCGGAAACCCAAATTCCCNTTTTT
CCCTNGGGANNCTTCTGNGTNAAAANCCCCCNNGTTTTTTTTAAGAAANCTNTTGNCCTNGGCTTTAA
AACCCCNTCCTTNCCNANANNGGGTTTGGGGCGTGGACCAAGGAAATTTTNGCCCATTANCCCAACGC
AAGGGTTTTNTGGGGGGAAGGTTCCCAAAAGCCAAANNNACNCNCGGGGGTGNGTGCCCCNNGAAAC
CCCCCCCCTNGGGTTTTNGGGGGNNNNNTTTTTCCNAAN
ANCAANACTGGAGCTCCCCGCGGTGGCGGCCGCTCTAGAACTAGTGGATCCCCCGGGCTGCAGGAATT
CGGCACGAGGCCTTGACGGGCCTCACCAGAGGGGCCACCTACAACATCATAGTGGAGGCAGTAAAAG
ACCAGCAGAGGCANAAGGTTCGCGAGGAGGTGGTTACCGNGGGCAATTCTGTTGACCAGGGCCTGAG
TCAGCCCACANATGACTCCTGCTTCNACCCCTACACGGTCTNCGATTATGCCATTGGAGAGGAGNGGN
ANCGATTGTCTGANTCTGGCTTTAAACTCTCGNGCCAGNGCTTATGCTTTGGCAGAAGGTCATTTNACA
TGCAATTCATCTANATGGTGCCNTGANAATGGAATGAACTNCCANATTGGATAGNAGNNNNATGGTA
AGGNATAGAATGGCCAAATGATGAGCTGNCTTNTCTTGGAANTGGACTTTGAGAGTTNAAGTGTGATC
CTCATGNGGCCGCNTGCTATCCGATGGGATGACTTACCACNNTGGNAGAACAGNGGNGGANGCGACT
GTCTTGGGGCCNTTTGCTTACTGCACATGCTTTGGAGGCCAACGAAGNTTGGCGCTTTGACAACTGAC
NNANACCTGGNGCTTGAACCCGGTACCTAANGCTTCCANTGGCCACTCCTACAACCAGTTTTTCCAGA
NNTTCCATCCGAGAACAAAACCTTAATGTCAACTGGGCCAATTGAGTGCTTCATTGCCTTTTGGATGTN
CAGGCTTGACCNTANAAAAATTCCCGAAAAGTAANTTTTTTCCAACCCCANAAAAAACAAGCGGTGG
GATTTTCTTGNCCAAGNTCCCTTNCCAAACTGGGAAGNGGNGNNTTCCCAANACCCCGCATNTTTAAG
GGGNGTTCCTTTTNNTTAAAACCCCTTTTTTNTTTTGGGAAANAAACTT
GCCTATNNGGAGCTNCANCNCAGTGGCGGCCGCTCTAGAACTAGTGGATCCCCCGGGCTGCAGGAATT
CGGCACGAGGAGAGATGGGAGGGTGATCAGACTGTCTCATGAGGGCCAGGAGCTTCAGGACTTCCTTC
TGTCTCAGATGTCACAGCATCAGGTGCACGCAGTTCAGCAGCTAGCCAAGGTTATGGGCTGGCAGGTC
CTGAGCTTCAGTAATCACGTGGGACTGGGGCCTGTAGAGAGCATTGGCAATGCCTGTGCCATAACTGT
GGCCTCCCCGAACGGTGACTATGCTATCTCAGTTCGTAATGGCCCTGAAAGTGGCAGCAAGATTATGG
TTCAGTTTCCCCGTAACCAGTGTAAAGACCTTCCAAAAAGTGATGTTTTACAAGATAGCAAGTGGAAT
CATCTTCGTGGGCCATTCAAAGAAGTTCAGTGGAATAAAATGGAAGGCCGAAACTTTGTTTTATAAAAT
GGAGCTGCTTATGTCTGCACTTAGTCCTTGTCTACTGTGATTTTTTTTTCTGGACCAGTTTCCAGATTTT
TTTTCCCCAGAGAAGTTTCCAGAAACTTTCAGGAATGTTTGCAGATCAACAATAAGCACAAAGAAGAG
GAATCTTCTAAAGGAATATTGTTTTTAAAAAGTAATAATTAGCAAATGTTTATTTAGCATTTTGAAACC
TTTCACTTTTATAAGTGACAAAGTGCTTTAAAATGCAAAAAAGTTTATGTACAGTTATAGGGTATACCA
ANCATGGAAGAGATGGTNCTTTTTTTATGCAGTTGGACTTGAAACATTACTACTATTGGATGGCATTAGC
TTTTTAAAAATATCCCNTTGATGTTGANATCCAAATAAAGATTTTGGTTAAAAAATCCNAACCCTNTGA
ACCCTTAAACCTAAATNNATTATNGCNTAACCATTCCCNTTGTTGGTNATGGCTTNAAAAAAANTTAA
AACCCCNTAAAAACCCAGCTTTTNACAAAAAATCT
NCCNATCTGGAGCTCCACCGCGGTGGCGGCCGCTCTAGAACTAGTGGATCCCCCGGGCTGCAGTTTTT
NANGAATGGCAGCTTTAATCATCCTGGCCAAGGCCCCTCAGGCTCCGTCCACTGCAGGCCTCCTTGGTC
ATAAACCCACCATCAACCATGCCTGAACACTTTTCCCAGGTGGCCCCTGCTCTTGGCCACTGACGTGGG
AACCTGAGGTCATGTCAGTCTGTGGATCTCTCCTGGGTGTGGATCCAGCAGCCCTGAGCCTGCTCTTGC
CATCTCCAGCAGCTCCTGGGGGACATGGCCAGCATCAGCGGGAGGCTGGTTGGGGAAGACAAGTGTT
CCCGCCACTGGAGCAGGCATTCCTCCTGGGCCGAGCTTCTTTGTGCTTTGAGACTGATGGAAGAGCGG
AGGTGCTGGGGCAGAGGCCCAGCCCAGCCCCTTGAGGCAGGAGAATCACCGAGAAGGCGGTTTCCTC
ACAGCACAGACAGGAGAACAGACACACGTGGGCACACACAGGCTCCACATCCTGTGCTGCTGCAGGG
GGCATGGCACAGCCCAGTGACCCTTCAAAGCTTCCAGGTAAGGATGCAAAGGAAGGCCTCAGGGAGT
GGACCANGCCCTGGCCCTGGGCCCCAAACTTTCCCAAAAAGAAGAACCCCAAGCCTGCAGGGGCATN
TTTGAGAAGCTAGAAGGGGAGCCCCCTNCAANANAAGGCCATGGCTTGACAAAGGGACCGGAACCCAA
CCAAGGAGCCTNTGNGACTTAACCGCCCCGANCATTGCTTTTTGCCAACTTTTGNGGGCTTTNTTTGGN
CCTNCCNCAACNAAGGGNNCANNNTGGGANAAAAACGGGTTTTTAAATTAACCCCCCCCTTCNGNTTT
GGGANGGNTTTTGGGCNCGGNTTGCCCNTTCCCTTTGGGGGGCCCNCCGGGGNGGGGGGGCCCTTTTN
GGNAANCNAAAATNNNTANTTNGNGGCCCCAAACANCACAAAAAAGNTTTTNGGAAAACCTTTTC
GCCCTATCNGGAGCTNCACCNCNGTGGCGGCCGCTCTAGAACTAGTGGATCCCCCGGGCTGCAGGNAN
AAATTGNNANCAATGCATTCATNGNCCTTCGAGCCCCATCAAACATTGNCATACATGATGAANTTGCG
GTTCCCTCCTGGGAATCTGCCTAGTCCTACAAATCCTCACAGGCCTATTCCTAGCAATACACTACACAT
CCNACACAACAACAGCATTCTCCTCTGTTACCCATATCTGCCGAGACGTGAACTACGGGGTGAATCAT
CCGATACATACACGCAAACGGAGCTTCAATGTTGTTTATCTGCTTATATNTGCACGTNNGACGAGGCTT
ATATTACGGGTCTTACACTTTTCTAGAAACNTGANATANTGGAGTNATCCTTNTGCTCACAGTAATAGC
CACAGCATTNATAGGATACGTCCTACCATGAGGACAAATATCATTCTGAGGAGCAACAGTNATCACCA
ACCTCTTATCAGCAATCCCATACATCGGCACAAATTTAGTCGAATGAATCTGANGCGGATTCTCAGTA
GACAAAGCAACCCTTACCCGAGTCTTCGCTTTCCATTTTATNCTTTNCATTTATNATCATAGCAATTGC
CATAGTCCACCTACTATTCCTCCACGANACANGCTCCAACAACCCNACANGGAATTTCCTCAGACGTA
GACAAAAATCCCNTTCCACCCCCTACTATACCTTTAANGACATCTTAGGGGGCCCCTCTTACTAATTCT
AGCTCTAATACTACCTAGNTNCTATTTCGCACCCCGACCCTCCTNGGGANAACCCCATATAACTTNCAC
CCCCAAGCCCNNTCCGNNTTNCAACCACACCCCCCTTCANATTNAAANCCCGGNGGGGGGNNNNNCTT
TTTTTGNCNTTACCGCNATTCCTTACGNATCCAATTCCCCCAACCAAACTTNGGGAAGGANGTTNCNC
GAGCCCATNNGGAGNNGCATNNCNGTGGCGGCCGCTCTAGAACTAGTGGATCCCCCGGGCTGCAGGA
TTTCGGNNCGANGGNNNTNCANGGGGTNTACCTCTGCCCCCAGGTNCTGNANCATCNGCTCCTACACC
AGTGGGCCCGGNTCCCGCATCAGCTCCTNGNCCNTTTCCCGAGTGGGCAGCAGNNGCNNCTNTCGGAN
GCGGNCTGGNCACCGGCATGAGCATGGCTGGAANCTACNGTGGGGCCCCAGGNTTGGGGGGCATCAC
AGCTGTCACGGTGAACCANANCCTGCTGAGCCCCCTCAAGCTGGAGGTGGATCCGAACATCCAGGCCG
TCCGCTCCCAGGAGAAGGAGCNNATCAAGACCCTCAACAACAAATTTGCCTCCTTCATCGACAAGGTG
CGGCACCTGGAGCAACAGAACAAGGTTCTGGAGAGCAAATTGAACCTCCTTGCAGCNNNNCNAAAGA
NTGNCNCGNNGCNACNTAANACNCCATGTTTNATAGCTACATTNACAACCTCCGTCGGCAGCTGNAAA
ACTTTTGGCCCCACGATAANCCTGAAGCTGGAAGTGGAGCTTGGCAACATGCAGGGGCTTGGTGGGAN
TGACTTNCNAGACCANTGTTTCGNCGGNTGAAAAANTNANANGCCGCACANACNATGNAGGANTGAC
ATTTCCNATCANTCAAGAANGANTGTGGGNCGAAAGNNTTACNTTGAAACNNGGNNAGAANCTTGGA
TTNCCCNCCCTGGGAAAGGGNTTGGACTTGGATNGAANANCNAANTTTCTTANNAGGCAANCCTGGTT
TGNAATAAGGNTCNTTCNNTNGTNNNAGNNAGCTCNTCANTAATTTTNTGGAACCCNTNAGNGGGGTC
TCTTGNCTCNTNGGNCAAANNANNTCNGGNAANCTTGGANCCTCNTGANTGGCNNTCNNTTTCTTTGC
TGGGCATNNGGNCCCCNNTTTTGTNANGGNGNATNGNNNAATCCCNNAATTTCGGGTTNGAANGCCCT
NTGGGCNNNTC
GAGCCCATNNGGAGNNGCATNNCNGTGGCGGCCGCTCTAGAACTAGTGGATCCGCCGGGCTGCAGGA
TTTCGGNNCGANGGNNNTNCANGGGGTNTACCTCTGCCCCCAGGTNCTGNANCATCNGCTCCTACACC
AGTGGGCCCGGNTCCCGCATCAGCTCCTNGNCCNTTTCCCGAGTGGGCAGCAGNNGCNNCTNTCGGAN
GCGGNCTGGNCACCGGCATGAGCATGGCTGGAANGTACNGTGGGGCCCCAGGNTTGGGGGGCATCAC
AGCTGTCACCGTGAACCANANCCTGCTGAGCCCCCTCAAGCTGGAGGTGGATCCCAACATCCAGGCCG
TCCGCTCCCAGGAGAAGGAGGNNATCAAGACCCTCAACAACAAATTTGCCTCGTTCATCGACAAGGTG
CGGCACCTGGAGCAACAGAACAAGGTTCTGGAGACCAAATTGAACCTCCTTGCAGCNNNNCNAAAGA
NTGNCNCGNNGCNACNTAANACNCCATGTTTNATAGCTACATTNACAACCTCCGTCGGCAGCTGNAAA
ACTTTTGGCCCCACGATAANCCTGAAGCTGGAAGTGGAGCTTGGCAACATGCAGGGGCTTGGTGGGAN
TGACTTNCNAGACCANTGTTTCGNCGGNTGAAAAANTNANANGCCGCACANACNATGNAGGANTGAC
ATTTCCNATCANTCAAGAANGANTGTGGGNCGAAAGNNTTACNTTGAAACNNGGNNAGAANCTTGGA
TTNCCCNCCCTGGGAAAGGGNTTGGACTTGGATNGAANANCNAANTTTCTTANNAGGCAANCCTGGTT
TGNAATAAGGNTCNTTCNNTNGTNNNAGNNAGCTCNTCANTAATTTTNTGGAACCCNTNAGNGGGGTC
TCTTGNCTCNTNGGNCAAANNANNTCNGGNAANCTTGGANCCTCNTGANTGGCNNTCNNTTTCTTTGC
TGGGCATNNGGNCCCCNNTTTTGTNANGGNGNATNGNNNAATCCCNNAATTTCGGGTTNGAANGCCCT
NTGGGCNNNTC
TTGGAAACNGCTGGAGCTCCCCGCGGTGGCGGCCGCTCTAGAACTAGTGGATACCCGGGCTGCAGGA
ATNANNAACCANGNTNTGCCCAAGAANCAAAAGCGGAATTGTNCCATNGGTCANTGGATTTCTAATGA
AAACTCGGCAATGAANTCANGTACAACTCCAAGAGAAGACACTTGGAGAAGAACCAAGCTGGGTNTA
TAANAAGCAAGCTGGNTCTATGAGAAGTGGTCTTAACATGTAGACCACTTTTTTAAGCAGCCAGATCA
CAATGAAAACATCACTACTGTAATGCTTGGCCCATGATGTTATTTCCTCACTATCAGTCTGAGACCCAG
CAATAAATATAANACGTTGCACAAAAAAAAAAAAANTNCTGNGGGGGGGNNGGGGCCCCNNCTGTTC
CNNTTTGGGNTTTTGANAAAAACATTNTTTTCANTTNTGGGNCTTTTNTTTNTAATNNTNNNAANTANTT
TTNNTCNCCTCAATTGTANGGTANCNCCTTTCTCCNTCNNNNNTACCGNTTNCNNTNTNNNNNNCCN
ANTCNNCTCNNCGANTTNCNTTTTNCTTTTCAGANTTAANTNNNNCNNNTGTTTCNNNNAGCCNNNGA
NTTTTCTNNNTTTCNTTTATTNTNNTGNGAGAGNNATNTATNANNGTNNATTTCNTNNCNTGATAGNN
GTTACACNNNNTTTTTNTNGCNTNAANCTACCCCCCCTTNGANANTNNTTTTTAANNGNNNNTTNAAC
NNTTNNGCGNTNTATTCNNTATTTGNATTTNCCTNGTGTTTCTTNTTNNGTCGNNTCTTANNTTCTCTCT
TTTTTACCNTANANNGTCCTNTNCNAGCNTCTTTGCCTTANNTTCCTTTTCTCGTNNTGTNAGCCTNGAC
ACCNCTAGANTTTTTTNTNTATTCGTNTTGGGGNNTNTCNNCTGANACNNTTTANTTTACTCNTTTNTN
NNTATACTNTTATTCTNGTAGTNNTTATCTATTCNNAATAAATCNTNAGGAGNTNANACCTACTCATT
NTANCTANNATGTGTTTTNTNTTNACNTCTNCT
AAAAGCTGGAGCTCCACCGCGGTGGCGGCCGCTCTAGAACTAGTGGATCCCCCGGGCTGCAGGGAGA
GGCTCCGCCTGTCCCCTAGTCCCACCTCGCAGCGCAGCCGAGGCCGGGCCTCCTCCCACTCGTCCGAG
ACACAGAGTGGGAGCAGCGTCACCAAAAAGCGCAAGCTGGAGTCCACCGAGAGCCGGAGCAGCTTCT
CCCAGCATGCTCGCACCAGCGGGCGCGTGGCCGTGGAGGAGGTGGACGAGGAAGGCAAGTTCGTGCG
CCTGCGCAACAAGTCCAACGAGGACCAGTCCATGGGCAACTGGCAGATCAAGCGCCAGAATGGAGAT
GACCCCCTGCTGACCTACCGCTTCCCACCAAAGTTCACTCTGAAGGCCGGGCAGGTGGTGACGATCTG
GGCTGCAGGAGCTGGGGCCACCCACAGCCCCCCTACTGACTTGGTGTGGAAGGCTCANAACACCTGGG
GCTGCGGAAACAGCCTGCGTACAGCTCTCATCAACTCCACTGGGGAAGAGGTGGCCATGCGCAAGCTG
GTGCGCTCAATGATCGTGGTCGAGGACGACAACGATGAGGATGGAGACGATCTGCTCCATCACCACCA
CGGNTCCCACGCAGGCAGCAGCTTGGGGGACCCCGNCGAGTACAACCTTGCGCTTCGCGCACCGTGCT
GTGCGGGAATTTGCGGGCAAGCCCGCAAAACAAGGCGTNTGGCCAACTTCGGGANNCCCAGNGGGCG
GATCCCNTCTCNTTTGGCTTCTTCCGCCTTCAGNGGGCACAGTCACTTCCAAGTTTTCCCAAAGTGTGG
GGGGGCAAANGGGGGGGGGGAAATTTTNGGGGNANAAGCTTGGTAAACCCGNTTTNTAACTTTTTNG
GGNAAATTCCNAAANCCCCNAAACCCAAAAACCCCCCCAANNTNGGGTNGGGGTTNNTTTNGGGGGG
GGNNTNAAAACAAANAAAAN
TNNNNCAAAAGCTGGAGCTCCACCGCGGTGGCGGCCGCTCTAGAACTAGTGGATCCCCCGGGCTGCA
GGAATTCGGCACGAGGCCTTGACGGGCGTCACCAGAGGGGCCACCTACAACATCATAGTGGAGGCAG
TAAAAGACCAGCAGAGGCAGAAGGTTCGCGAGGAGGTGGTTACCGTGGGCAATTCTGTTGACCAGGG
CCTGAGTCAGCCCACAGATGACTCCTGCTTCGACCCCTACACGGTCTCCCATTATGCCATGGAGAGG
AGTGGGAGCGATTGTCTGATTCTGGCTTTAAACTCTCGTGCCAGTGCTTAGGCTTTGGCAGTGGTCATT
TCAGATGCGATTCATCTAAATGGTGCCATGACAATGGAGTGAACTACAAGATTGGAGAGAAGTGGGA
TCGTCAGGGAGAGAATGGCCAGATGATGAGCTGCACGTGTCTTGGAAATGGAAAANGAGAATTCAAG
TGTGATCCTCATGANGCCACATGCTATGACGATGGGAAGACTTACCACGTTGGGAAAACAGTGGCAGA
AGGAATATCTTGGTGCCATTTGCTCCTGCACATGCTTTGGANGCCAGCGGGGCTGGCGCTTGTGACACT
GNCGCAAACCTTGGGGCTGAACCCCGGTACCGAAGGCTTCACTTGNCCACTCNTACAACCAGTNTTTC
CCAGAGATNCCNTCAGAAAACCAACNCTTATGGTCACTGGGCCAATTGAGNGCTTCATTGGGTTTGGA
TGTNCAGGCTGACCNAAAAAAATTTCCCGANAGTATTTTTTTCCAACCCCNAAAAAACAAGCGGNGGG
NTTTTTTGGCCAAGGGNCCTTTCCAAACTGGGNNNGGGGTTTCCCNAANCGCCNAGGTTANGGGGGGT
TCCNTTTTTTTAAANCCCNTTTGGNTTTGGGAANAN
NNTATCTGGAGCTCGACCGCGGTGGCGGCCGGTCTAGAACTAGTGGATCCCCCGGGCTGCAGGNNNNN
NGCNCNNNGGTCCTGGNNNNATGTGAGAANNNNGNCAANCNCTGNCNGGCTGNTGCCNTGGNCNTGN
ACNTCNNNACNNNTNAGGNGCNNCACACNCTGGCTNGNACNNNCGCANATGNCCTTACNGNNNCTNG
GACCNAAATGNNNGNCANGACCCGNNACTNNAACNGNNTACANGNTGNCATCNGGGNTNTGNNANN
CCTGNCGGNTTACNNGNTGGCTNNNANNGNTCANGCTTNGCAAGGGNNTNAGATGACTNNTANAGAT
NCNANGNCNAANCNGACCNNGNTNCTGGCCAGGATTNACNAACGCCCTCTGCTACATGGTNAGTGCT
GTGNGAGAGNNCNATGAAGTNANNTTTGTATTNNTGGCCCTNNNNGNTNNNANTNCCAGNNACTGGA
AGNTNATGATNNGNTATGAGAGCNNNCNNGNGTCTNACCTGCGCNTACTNNATNNNCGNANCCTGAN
CNCNCACGNNTACTTNCNTNGNNTGACNTCGNACTACGGGACNCNTGCTTCANNTACAGACTGNNCCC
TTGTTNACTGAATCNACCNAGATTGCGANCCTGNCNTNNTANNNANTATNCNTANCNTGCNTGGNGAA
GANANCGTNCTTATNACTNGCTGACCTNTTTCCCGNNNANAGTNGCTTGCATTTCNCTGCNACCCTATC
AAGAATGNTTTGGNTTACNAGNAAAACNNACCTCCGCCCCNNNTAAANATNTGGCGCGANCTNGAAN
CANATNGTNTTTGCNCCTGTTNTAAGCCCGNNACNTGGGGGNNATTTATANTGCNTTGCTTTTTGAN
NCTTTTNGGCCCTTTTCNTCCNCNAAAAGCCAAAAGCAATTNNGNTTNTTGGAAACNTCCTTTGGGNG
NGCTTGNNTCCCCCCNCGNTGGANTNGGNANGTTTTTTGGGNTNNGGTGGGCCNTNTTAAAANTTN
CCCCCCNCTCTCTNTNCCGCTNTNTTTTCTGNTTNTTNTNNNTTNNNNTCTCNTCNCCCCTCCNGNCNTN
TTTCNCTNGCCCNGCGGCGCCCTCTNCNTCCNCCNTGNGGCCTCNNNNNCANATGNNTGNNNNNNNNT
TTNAGCCCTTTCAGTGGAGTCGTATTNCNAGNGGAGNCCGCTCTAANAACTAGTGGATCCCCNCGGGN
CNTCNNGTCNTCTNGGNTNNGNTNTTTTTTCCTNCTCNGNNACCTCTNGTNTTTTTNNCCCCNGGGGGN
NCCCTNCCTCCTTTGNNCTTTCCTCNCGCCCNCCTNTTTCCGNGTTTTCTCCTNCNCCTGATTCTCGTCNC
GNCCNTACTCCTGGTTTGNCNTTNTTCCCTGTNCNNTCTTCTNCTCTTNCNCGCGTNTCNCNGNCCCNN
CTACCTCCGCCCNTNNNNCNCNNTTTNTTNCNCGNCCCNGTTCCTGNNCTCTNGNGTNCCTNCCCCCTCN
TCCTCCNTTNCCCTNGTNCNNACNNCTNCCTNCNNCTTANNCTNGCGGCCTCNTCCCCNNCNNCNCCN
NNCCTCCTCGNTCCCCNCNCCNCTTTCTCCCGCNCCNNTCNCTCCCGGCNTCCCTTCCTCCCTCCTTNCT
CCCTTCCCCNCNTGNCNNCTNNNCNCCCTNTCNGNCCCCGANANNNNNTTNNCCTCNGGCCCCNCNCT
CCCNCCNNTTANNTNTCCCCCTCCGTCCACGCGCCTCCTCTCNCGTCGNNCCNNTAGNCNNTTCCGNNC
NGGCCGCCNCCNGTTNCTNCTCNTCCCNANTTCNTCACTNGCGNTGCCGNNTNCNNGNCANTGNNCTN
NNNTCNCCTCGNGTCCNCCTNNNTNNTNGCNCTTAGTCCCTCTATNTNNNTCNNNCCCGTCTCCNCTCT
CGNNTTCNATNTTNCGTCTNCGNNGNNNCNCNATGTNCTCTNCGCCGNNTTGTCNAATCNTNCNCNNN
TCTNTCNCCGTCGTGGCNCNTGCNNNCNCCCTNCAANTCTCCCACTTTTTCCCTTTCCNNCNTCCTTCNT
CNTNNTNTTTCTCCGNNNTTNCCCGTTCNTGCCNTCCCNGGTCNCCCTCAGCCTCNCTNTTNCCCNCCTT
CCCNCGGNCCTCNCGACTNCCTCCTGNCGCCNCTCCTNTGNTGNTGNNNTCCNCTCNTCTACNNCCNNCN
CTNNCCCNCCTCNNNNTACTTCGTATGGNGCTCNNANCTCTTCNNCGCTNCCCTNTTTCTACNTNCCTC
NTCTNNNNGCTNTNTTCCGCCGTNTNTTNCATTGTANGNGGNCTCTCTCCGNCNTCNCCTCNTTCNNGT
CTCNNCTCTCTCTCTNCNCNNTTNTNNNNCCTCCCCGCNCGTCGGTTCTCCNTNTNTTNTGGCACTCNN
CGTCTGNTCGCCTCGCTNCNTNGNNTNCNCTCGTNCCGGTTTCCCCNCTTCTCTNTTCCNNTNACTGTC
NNCTCCCCACNNACCNNTGCNTNCTNCNTNCCCTCCNTNTCCCCCCNGCGTGNNCCTCCCNNCGNGTC
TNTCNCTCATCTCNNCTTGGGTCTGACGCNTNCGCTAAGGNNNCNCNCCNATNCCNTNGCTCNCTCCG
CNCTCTTCCNTCCTTNCNGTNNCGTANNGCACCNACATNTTNGTGANCCCNTCGCTCTGGNCNCCNCCT
CNCCGCTTNGTGCTCTACTCGNTCCGCCNCNCTNTNCTCCGTCTCTCCCNCGCGC
GCCCTATAGGGAGNCGNATTACAGTGGCGGCCGCTCTAGAACTAGTGGATCCCCCGGGCTGCAGGAAT
TTCGGTACGAGGATTNTTGTCANCCTGNCGACCNGGANGGGGGGTCNNGGGGANGCAATTTNGCCCA
TATNNTGATCTACNTCTTACNNCGTTTTTTAAATCCGCATGAGANGTCCCAGTGACACNGNGAGACNA
TNCACAGNGNTCNTNGNNNGCTNCGAATGACCNCTTGNNNGNNATCATGACCAGNCCGANGGNTGAT
TNTAGAATTGCCTGTGGGACCAGCTAACNNTCNNGCTGNGGCNNNGCNGTAAGTATNTGNNTGNANT
GNCCCNAATCTANCTGNNGANGGACTNAACTTTGNNTATTNNNNNCATCTGNNACNCGNANCANCAG
CAGAGANGCNTCTGNGNNTNGAACGACNNNCTCTGTGNATATANNNCAATTNNGGGGGNACANCTGC
CANANCACCTNNNTTGNNTANTTGNGCATANCTGNAAAGGNANTNNNTTCNNCTCTNACCGTNGGGCT
NTTNAACNAACNCNTTGNTNCAGGNGTGCNTGNTCTNTNNGANACNNGANTGNNNTNNNCCNCNNNT
CGNANCNNATNNNCANGNANGANANGNANGNGGCCAGTNANTNNNGNNTCTTANACCNANTCCCNN
TGCACTACTTCTTAGCNNAACCTNCTGGNCTTNAANNNNTNCNNTTAAGAANTNTNATNTTTCTACTGT
CAGNNAANTTAATCGTCNNNNNGAATNANTTNTCCNNGGGGCNNGAAANAATTANCTAANTCCNTTA
NTGNTCCACNTNNAAGGTGCCTGNTCCCCTNGNANCGAAAANGANCTTCNTNCACTTGGGNNNCGNN
CNNTNAATTNNCAAAATNANNTNCNNNNTNNTTTNTTCTGNGGTGGTTGTTGTTNTTNNAACNANTGC
NNTCNTNNNCAGNGGNNAGGATNTCTCCTGNATNACTTCNTAGAGNAANTNATCTNNCNANCNANNN
TTCNTCNAANAGGNCTATTANCNGTCCTNCGNTANANTNGNNAANNAGNCCAGNACCCCACTGTNNT
GTCTGNGNTTNNNNNTATNNNTNGNNNNNCCG
NCNNNATCTGGAGCTCCANCNCNGTGGCGGCCGCTCTAGAACTAGTGGATCCCCCGGGCTGCAGGANT
TCGGCACGAGGGTCCTACAAGGTGTCTACCTCTGCCGCCAGGTCCTTNANCANCNGTTCCTACACNATT
GGCCCCGGTTCCGNATTAANTTCTTGGNCNTTTNCCNANNGGGCNNNNACAACCNNTTTTGNGGGGGN
CNNGGCNCCNGNNTNAACNTNGGTNGAANCTNCCGGGGGGNCCCANGNTTGGGGGGCNTTACANCTN
NCACCNNGAACCAAAACCTGNTNANCCCCCTTAAGCTGGANGNGGATNCCAACATTCAAGCGGTCCG
CACCCAGGAGAAGGAGCCGATCAAGACCCTTAACACACACAATTTGCCTCNTCTTTATGACAAGGTGC
GNCACCTGTGAGCACAAAGAANANGGTNCTTGNAGACACCATNTGAACTCNTGNNCCATACATAAAA
NNTGTGCGGAGCNANATATACANCATGTNTGTGAGNTATATTNANAACCTCTGTCGGCAGNTGNNAAC
TCTGNNCGACGAGAAACTNNAGACTGTAAAATGNNGCTTGTCNCATGTAGGGGCTGGTGGGGGACTN
TTCAACAACGATTGNGGATGATGATANNAANCNCCANAAATCGNGGAATGAATTTTTCTCATCAAANA
ANGATGTGGATGAAGCTTNTNTGANCAACGTGGAACTGNNGNCCCCCCCTGCNANGGGCTGCTTGATG
GAAAANAACTTTTTCCAGCCGCTGNNTTAAAAAAGAAAATCCTTNNNAAANNAGCCTNACTNTTTNTTT
TCTTACCCCCNNGGGTCCTTTTTCTTGGAACAAACAACGCCCANCTTGGANCTTGGATNGGGNTTNATT
CCNTTGNNGGGNANNGGGCCCCCCTTTGAAGGAAAANNNCNCNCCCCCCCCCNGNCTTNATACCNAA
GNCNTTTTTCN
NCNNNATCTGGAGCTCCANCNCNGTGGCGGCCGCTCTAGAACTAGTGGATCCCCCGGGCTGCAGGANT
TCGGCACGAGGGTCCTACAAGGTGTCTACCTCTGCCCCCAGGTCCTTNANCANCNGTTCCTACACNATT
GGCCCCGGTTCCGNATTAANTTCTTGGNCNTTTNCCNANNGGGCNNNNACAACCNNTTTTGNGGGGGN
CNNGGCNCCNGNNTNAACNTNGGTNGAANCTNCCGGGGGGNCCCANGNTTGGGGGGCNTTACANCTN
NCACCNNGAACCAAAACCTGNTNANCCCCCTTAAGCTGGANGNGGATNCCAACATTCAAGCCGTCCG
CACGCAGGAGAAGGAGCCGATCAAGACCCTTAACACACACAATTTGCCTCNTCTTTATGACAAGGTGC
GNCACCTGTGAGCACAAAGAANANGGTNCTTGNAGACACCATNTGAACTCNTGNNCCATACATAAAA
NNTGTGCGGAGCNANATATACANCATGTNTGTGAGNTATATTNANAACCTCTGTCGGCAGNTGNNAAC
TCTGNNCCACGAGAAACTNNAGACTGTAAAATGNNGCTTGTCNCATGTAGGGGCTGGTGGGGGACTN
TTCAACAACGATTGNGGATGATGATANNAANCNCCANAAATCGNGGAATGAATTTTTCTCATCAAANA
ANGATGTGGATGAAGCTTNTNTGANCAACGTGGAACTGNNGNCCCCCCCTGCNANGGGCTGCTTGATG
GAAAANAACTTTTTCCAGCCGCTGNNTTAAAAAAGAAAATCCTTNNNAAANNAGCCTNACTNTTNTTT
TCTTACCCCCNNGGGTCCTTTTTCTTGGAACAAACAACCCCCANCTTGGANCTTGGATNGGGNTTATT
CCNTTGNNGGGNANNGGGCCCCCCTTTGAAGGAAAANNNCNCNCCCCCCCCCNGNCTTNATACCNAA
GNCNTTTTTCN
TNNNNAAAGCTGGAGCTCCACCGCGGTGGCGGCCGCTCTAGAACTAGTGGATCCCCCGGGCTGCAGG
CCTGACCTGGCAGGCCTGGAGGCGCTGCGGCGGCGGGAGCAGCTCCCCAGCCTGGCCCAGCAGGAGG
ACCCAGAAAAGGTAAAACTTCTGCCTGAAGTTCAACTGTGCCTCGAGACGTGTGGTCCTGAATGCCAG
CAGAGCTATGGGAAGGTGGAAAGTACACAGCTCGACAACGAGTCCCAGTGAGGCCAGCCTCCGGCCT
GACAGCAGAGGAACCAACCCAACCTAACATGTCACCAGTGTCAAGGATGCTCCAGGTCCTTGCAGGA
CCCTGGCACTTCCCACCCAGTTAGATTTTGCTCGACAAAGCCTCTCTTTCATTCTCCAAAGCATCCACG
GGCTTCACATTTCCTCCAGAGGCCTCCCAGGAANAACGCTNGATGGTGTCTCCCTGCTGGCANANAAA
AGCCCTGGNAGCTGGTGGCCTGCACCAGTGCGTGATGTGGGGTGACNTGNCGGCCGGGGGGACCCCC
GCGGGGGCCTCAAAAAGCTCANAGAAAGGAAGCNCCNTTCACAAAGATACCTTCCCCCCGCCCCAGC
CTGTTGAGCCGNAAACACCCAGCCATCCTGAAAGCCCACAGNTTTGCCAAGGACTTTCCTTGTNGGGN
GGGGNCCCCCTTGGGCTTCATTAAAATAAAAAAGTCGTCGNGGCAACCCCCTTNAAAAAAAAGGNG
GGCCNAANTTTNGGGNAAATCGGTTGCCTTTTTTTTGGGCTGGACCTTGCCCCCCAATTGGGGGTTTTG
CCCGGGCAAGGGGNCCCGGGCCGGGCCCTTCCNGGGCTTTCNTTTGGGGCACGCCTAATTGGNCCTGG
GNNNNGGGCCCCCCCCCCCTAAACAAAAAACANGGCCTTTAAANCCNCGGGGGGACNTTTTTAACNC
CCCTTTTGGGGGGGGGGTTNCCCNTTTGGCCNTTTTCNCCCA
TTNAAAAAACTGGACTCCCCGCGGTGGCGGCCGCTCTAGAACTAGTGGATCCCCCGGGCTGCAGGGTT
TGGTGGACTCAGACAGNCCATAGTGGCCTTTTGACCTTGAGCAAGGGGCTCAACCTCTCTGAGCTTCT
GTCTCTTCTGCAAAGCGGAGCTCTTAATAGTGGTAACCCACAACGGCGAGGAAGCCAGCCAGTTTNTG
GGTAAAACCCTTGGCGTNCATTAAAGCTGATTTCTCCTCACTGGCCCTGTGAAGCAGGTTCAGTTATTA
CTCTACGAGATAGATGGGGAAGCCAAGGCTGANAGAAGGTTTGGGGCTGGGACCCTAAACACCTGAG
TGCAANACTGGCCCCCCTCCACCGAGGGAAGTGGGCTGAAGCCTGGCCCAGGCGCACCCCTCCNTGGG
GAGAGCCTGGGATGCCCTTCAGCCTTAGTGGCTCCTGACTCATGGAGCCCTCAGCCACCCCAGGGCCC
CAGATGGGGGTCCCCACGGAGGGCAGGGAACGGTCCCCGGAGCCTCCANACTATGAANACNAGTTTC
TCCGNTNTCTGNGGCGTGATTATCTGGTACCCGAAGCAGTACCAAGNGGGTCCTNATCGNANCCTACG
TGGNTTGTGTTNTTCGAANCCCTGGGGGGCAAACACACTTGGTGTGCCTGGCCGTGTGGCNCANCCCG
CNTGANGGACGGNCACCAAATTACTTTATTGGNAAACCTGGTCCGTGGGTTGACGTGCNTGGNGAACG
GCCATTTTTGCCTTGCCGGNTAACNTTNTTAATAAAAAAATCACNTGGAATCCTTGGGTTTTTTTGGGC
CATTGCCCNTTTGGGAANGGGCAATTCCCCNTATTTTACANGGNCCGGTTTTTTGTTTCCCCNGGGCAA
AGGCTTTAANCGGTTAAATTTTTNTTTNNCCTTTGGGACGCGGTTGGGNTTTGCCANTTTTGGCCCNCC
CTTTTGNTTTTTNAAAAAAACCA
CAAAAGCTGGAGCTCCACCGCGGTGGCGGCCGCTCTAGAACTAGTGGATCCCCCGGGCTGCAGGTTTT
TGCCAGCCTCAGACATACTGGTTACATCTGTATTGAACTTTTGACCCTAGAAACCAATGGAGTTATTTC
ACCAGAAATAACGAATATGCCTGAGGTGCATGGGAATATAGTTAGCTGTACTCTGAAGATACATTTTT
TTTTTTTTANCTAGGGCCCNCANCNTNTAACCNNGTAAAAGNAAAAANCNGTNNNAAANGTNCNTTTT
TTNAGTNCNCCAANTGGNAACCNTTTNGCNGNTNNGGGGNTAAAANTTTCTTTGGGCCANTTANCTAT
NGGATTNATNTTNATNATAANNGGTNATTAAATTTTTTTCCNAATTTANCCNGNNTANCNAANCGGGG
TTNNCNAANANNGCCCAAATNGTANGGTNGCCNGNTTCANNAAAANGGGTNTTNGGNANNANTNTTA
NAANCCNTNNCAAATTTNANGCNGGANTNNACAACNTCNTCAATTTNTNATAAAAAAAATCTTTNGA
ACCCAANGGGATNAATTANGGGTTATTAANAATTGGNTTGGANATCNNACNGGGGCAAATTTTTTAAA
AGGAGNAAATTGGCCTTTATAANGNAAATNGGGGATTTTGGTTNNACANGGGGGTTCCTATAGGTTTA
AATTTTGANNCTTTTNAAAAAACAAAAAGGGTTNGGGGAAATTGGGNANAAATTTTNAAANGGGTACC
GGTNTTTTAAAAAGCNCAAAAATTTTCCCCTTGGGAANNGGCCTTTTAAATNCCTTTTAAAAAATTNTN
TNNTTTTAAAATCTANGGGGAAANTTTTCCNCGGAAAAATTTTTTTCANGGGGTCAAAAAANCCCTTT
NNTTTTTTNAACATTTGGGGCCCCGGGNCCGGGGGGGGNTTGNCCCCANAAGGGGCCTTTGNAAAAA
AATTTAANTTAAATTNNT
TTNANCAANCGCTGGAGCTCCACNCGCGGTGGCGGCCGCTCTAGAACTAAGTGGATCCCCCGGGCTGC
AGGAATTCGGCACGAGGGTTTGATATACCTTCTATTTTTATGGAGAACATAAGTTTAATTGGTGTTTTG
TGTTTCTAAGCAATTGACTAATAAAACACAAAGTTGGTTAGTAATTATTTTGTTAACTTGGTGAAGTCA
AGTATAACTATTATTTATTACTTGTACATTNGATAAGAAAATTTTCAGATTTTGAATNGCACAGATCAC
ATTATATCTATTGCATTACTTTTCTATACTTCTAACTGGTCTCTACTCTGGTTGATATTTTTTGTAGTTAA
CTTTACCTATGAAAACTTTTAAGGGTTTATAAGACTCAACCAGAACTACATATCTGGTGATTATCTCAT
TAATTAACAGCATAAAATTTATACTATAATGTTTCAGTCANTATAATTCCACTTAATAATTAGTGAAAA
TTTCATATTATCCTAAGTATTACAGTATTAGTCCATTTTTTTCTTCTCCTAAATGAAGGGGTGGTTTATT
TTGGTCTGGGGGTTATATCATTTTAACCTTCTTTTAATTCTTTTTTGGGCTGGGGGNAATATTTNTANATNA
CNGACCCCCTCTAACTTACANAGNNANTAGAGACATTTAAAAAAACCTNGNGGGGGGGCCCGGNNCC
CCAATTCNCCCTATANGGGGGGNGGGNAAAAANNNNNNNNNNNNNNNNNNNNNNNNNNNNNNNNN
NGGNCNNNANAANAANNCCCNNNNANNNNNNNTNNNNCCNNCCCNNNANAAANCNNNNNNNNGNNN
NNNNNNNCCNNNGGNNCCNNNCNNNNNNNGGNNNNGGNNNNNNNNNNNNNCCCCCNNNNNNNNNN
GNNNNNNCNTTNNNTNAANNCCCNTTTNNNNNNNGGNANNNN
AAANCTGGAGCTGCACCGCGGTGGCGGCCGCTCTAGAACTAGTGGATCCCCCGGGCTGGAGGAATTCG
GCACGAGGGGAATATNTTTGGTGCCATTTGCTCCTGCACATGCTTTGGAGGCCAGCGGGGCTGGCGCTG
TGACAACTGCCGCAGACCTGGGGCTGAACCCGGTAACGAAGGCTCCACTGCCCACTCCTACAACCAGT
ATTCCCAGAGATACCATCAGAGAACAAACACTAATGTCAACTGCCGAATTGAGTGCTTCATGCCTTTG
GATGTACAGGCTGACAGAGAANATTCCCGAGAGTAATATTTCCAACCCAGAGAAACAAGCGTGGATC
TCTGCCAAGGTCCATCCAAACTGGAGTGGTGTTCGCAGACCCCATGTTAGTGTGTTCCTTTCTTAAGCC
CTTTGCTCTGGAGTAACTTTCTCCAGCTTCAGCTCAACTCACAGCTTCTCCAAGCATCACCCTGGGAGTG
TTTTCAGACTTTTCTCATACATGGGAGCAGTANGTTATCTATTCTTCAAAATATTCCATGCCAATCAGT
ATTTTAAATGAAGTGNTTCTAACTTGTGATTNGGATTGGGATCCATAGGGAAGCACATGCACCAACCA
AGATACAAAATGTCTTGAAATGATATTACCAAAATTNTAAGTAGGAAAGTTACCTGAACACTTCTGCT
TNCACTTAACTGACTGGCCCGCAATATTGTANGAAACAAGCATGGTCCTTTGTACTGTGGNATTCAAA
ACAGCCACAAGTACTCACTTTTTCCAAAAGGAATCTAGTAATTGGCTAAAAAAATCTTTTTCNTTTAAC
TGATTAATCAAATTTTTCCAGTATTTTTTATATGGGAAAAAAAATGGACTGAANANACNTAATATGCA
GTNGATAANAAGGNAACTGGGCCNAATATGGGGGGGGATATTTTTTATACCGGATGGGNCCAAGCTTT
TNTACTGGGGNAAANAAACTGGTTTAANT
NCCTACNGGAGCTNCACCGCNGTGGCGGCCGCTCTAGAACTAGTGGATCCCCCGGGCTGCAGGGTGA
AAGGGAACTGTTTAGTTTTAACAAGACTAAGTTAATCATGGGAGCCCTCAGGTGAAAGAGTTTTAGTA
ACAACAACTCCTCTTTCCCATAGTCAAGGTGGATTTGTCAGGCCCACCTGAAATGTGCCCATAGTCAA
ATCATTTACTCGCTCCTCCTANATCGCTGCCCTTGACGTTTCCCATTGCTGTACGTATTTCTATGTATCT
GTTTTTCTCTTAGAATGTCCACCTCTTGGGACTTGCTTAGATGACTGAATATGAATATTATTGGTATTAG
ACAATAATTTGCTTTCCATCCAGTATGCTAGTTCTTATTCAAGAACTGTGGTCAGAGGGTATTTGTATA
TGAGTATCCTTTGCTTGTCTTTCTAGNACTGAAAATTGACAGAAGTAACTGGCTGTGCAGAATGTAATA
GAAGCTTTTCATATTCTTTTATTCTTAAAATCAGTATCTTTTTACAGTATTGTTTGTACATGATCCTTTTT
TGTACATTTAAGAATATTTTGATTACATTAAATAAGACTGCTGATTTTGCTACTTTTTTTAAGGGGTCTT
CAAGTAAGTAAAAAACATACATTATAGGGTAGAGGGAAATGGACCTTGAATTTCTATCTTCTCTCCCA
CACTAAAGCTGNAAACACTTTGACATATTTTGNNCTGAAACACTTGGGTCAAATATATACCTTNTTGG
GTTTAAAAACCTGGTNCCAATCAAANTNTTTTTTTTTAAAATTTTAAAAGGCTNGGNGGAAAATNAAC
CCTTTTTGGANGGAAAAAAGGGGGGCCTCCAAAATTTTAGGGAAGGGATNTGAATAGGGCCCAGNNT
TGCNNAANNCNNATANTTAAAATTTTCTNAANTTNNCCNCCCNAAAAAAANGGGGGGNNGGGGGCCA
NTAAAAAAAAAAAACTGGTTNNNNNGGANGCTAAGGGNNNNNCAANGGNCCCCG
GCCCTATNNGGAGNNGNATCNCAGTGGCGGCCGCTCTAGAACTAGTGGATCCCCCGGGCTGCAGGCA
AAATCAACACCGATGAGATTATGACCTCACTCAATCAGTCAATGGACAAATAGAAAGCCTCATTAGT
CCTGATGGNTCCCGNAAAAACCCTGNACGGAACTGNAGGGACCTGAAATTCTGNCNTTCTGAACTCCA
GANNGGAGAATATTGGGTTGATCCTAACCNAGGGTGCNAATTGGATGCTNTTAAAGNCTACTGGAAC
ATGGAAACTGGGGAAACGTGCNTNAGTGCCNNTCCTTTGACTATNCCACANAANAACNGGNGGACNG
ATTCTGGTGCTGANAANAAAGNTGTTTGGTTTGGANAATCCNTGGAGGGTGGNTTTCANTTTANCTAT
GGCAATCCTGAACTTCCCGAAGACNTCCTCGATGTCCANCTGGCATTCCTCCGACTTCTCTCCAGCCGG
GCCTCTCAGAACATCACATATCACTGCNCAGAATAGCATTGCNTACATGGATCATGCCAGTGGGGAAT
GTAAAGAAAGCCTTGAAGCTGATGGGGTCANATGAAGGTGAATTCANGGCTGAAGGANATAGGANCA
TTCACATACACAGTTCTGGAGGATGGTGTGCACAAAAACACACTGNGGAATGGNGGCCAAAAACAGT
CCTTTCAGTATCNAAACACGCANGGCCGGCCAGACTACCCTATTGTAGAATATTTGCACCCTATTGAN
ATTCGGGGGGGGCNTGATCAAAAAATTTGGGTGGCCGGACCATTNGNCCCCTGGTNTTGCTTTTTTTTN
AAACCCAAACTTCTTTTCCAAAGNNCCCACAAAAAAGCCTTCCCCCCTNCCATTATNGTTTCCCTTTTT
GGTTNCTTAAATCCTTGGTNAGNCCNGCTACCAAGTNGNCCCAAACNTTCCCANTTTTTTTNTTTTTCC
CCNAAAATNTCTTTGGNGGAAAAAA
AAANCTGGAGCTCCACCGCGGTGGCGGCCGGTCTAGAACTAGTGGATCCCCCGGCCTGCAGGGAAAA
AAGAAGGTTCCTGCTGTGCCAGAAACCCTTAAGAAAAANNCGGANAAATTTCNCAGAANCTNANAAT
NANCCNACNGAAAANAANTTGNCCCAAANAANNNTNCAAAGGNCAGGGNGNANCNTANTTNTTAA
AAANCNTANCCTTNCCCCCAGGNATNCCNGCCNANNCCNAACNNNAATTNCAATGGNTTNGANGG
NNCCAAAAGCCNGTNACTTNTNTNNCCCNCNGAACCCNAATTGGCGTTTGGCATTCNGANNANANG
NATNAATGGTGTGANCCCGAAGGGTCNAAAGGGCTGCANCTTCTTTCCTTCGNCNGATATTTNCGGGG
CCTTTTGANNCTCACCACGGCGTNAATTNCATGCTCTGAANNNTGGAGGCCTACANNNNNTGNGGGNN
CACNNATNTGAAATCTGTGAATGANNTNANTTACACNCGNGGNTCGNCGNAATCTCCACAAAGCGAA
TTNCCTGNCACACAANGCATNNATTGCTCNATCTCTTGGGAAANATGGAATCATCTGCNTGGAGGATC
TNATNCATGANGACATATNCNCGGGANACGTTTNAAAAAAGCAAACACTTCCTTNTGGNCCNTTAAAA
NTCTTCTTCNCNACGGGGGGGTGAAGAAAAAAAACCCCCCCTTTTTTGAAAGAGGGGGANGCTTGCTG
GCAGNGGAGAACACCTNAACNGGCTTTTTTATAAGGANGAACTACNGNGTTTCCNGGAAGATTATTGG
NNGTCNGGGTGGTTAAACAACCNNACCCAAAAAAAAAAAAAAAAANNTNNGGGGGGGGGGCGGGCC
CCCNNNNNNNNNNTTTTTTTNNGGGGGGNNNTNTTTNNNNNNNNNNNNNN
AAANCTGGAGCTCCACCGCGGTGGCGGCCGCTCTAGAACTAGTGGATCCCCCGGCCTGCAGGGAAAA
AAGAAGGTTCCTGCTGTGCCAGAAACCCTTAAGAAAAANCGGANAAATTTCNCAGAANCTNANAAT
NANCCNACNGAAAANAANNTTGNCCCAAANAANNNTNCAAAGGNCAGGGNGNANCNTANTTNTTAA
AAANCNTANCCTTNCCCCCAGGNATNCCNGCCNANNNCCNAACNNNAATTNCAATGGNTTNGANGG
NNCCAAAAGCCNGTNACTTNTNTNNCCCNCNGAACCCNAATTGGCGTTTGGCATTCNGANNANANG
NATNAATGGTGTGANCCCCAAGGGTCNAAAGGGCTGCANCTTCTTTCCTTCGNCNGATATTTNCGGGG
GCTTTTGANNCTCACCACGGCGTNAATTNCATGCTCTGAANNNTGGAGCCCTACANNNNNTGNGGGNN
CACNNATNTGAAATCTGTGAATGANNTNANTTACACNCGNGGNTCGNCGNAATCTCCACAAAGCGAA
TTNCCTGNCACAGAANGCATNNATTGCTCNATCTCTTGGGAAANATGGAATCATCTGCNTGGAGGATC
TNATNCATGANGACATATNCNCGGGANACGTTTNAAAAAAGCAAACACTTCCTTNTGGNCCNTTAAAA
NTCTTCTTCNCNACGGGGGGGTGAAGAAAAAAAACCCCCCCTTTTTTGAAAGAGGGGGANGCTTGCTG
CCAGNGGAGAACACCTNAACNGGCTTTTTTATAAGGANGAACTACNGNGTTTCCNGGAAGATTATTGG
NNGTCNGGGTGGTTAAACAACCNNACCCAAAAAAAAAAAAAAAAANNTNNGGGGGGGGGGCGGGCC
CCCNNNNNNNNNNTTTTTTTNNGGGGGGNNNTNTTTNNNNNNNNNNNNNN
TTGAANAACNATCTGGAGCTCCACTCGCGGTGGCGGCCGCTCTAGAACTAGTGGATCCCCCGGGCTGC
AGGANTNNGGNNCGAGGCCTTGGTCCAGCGCTCGAGGNTGNTCTGCCTNCTGTTNNATNCNTTNNTTN
TTCTCCTACTNGCTGANCGCCNTCGGGGGCAGCTCCGCANCCCGCGCCANGGTCTTCANNNTNANCCN
GCANANCTTGNGCNTNGTGGNANACGACGATTGGCTCANNANGGGNTATCGGTGAGCTCATNGGTAA
GNNCATCTATGACTTCTTTGCTCTGGNTATCTTAAAGCTGGNCACTGTGCNGGTTGNTGNNGTTGTGGG
CNCCTCCNATGCCCCATGATTTNGCGACANTCCGCTGCAGGAATCTTGCTCCNTCCTTGGGGNTNCGN
NAANANCGAGGAGAATAAAGAAGAGTTNGANGANCTCNGANGAATGACCNTGGGCTTTGGNTTGNTT
TNACTAGAATCCCCGCTNCCCTGGGAAATANNACCCCTTTTTNTGTNAAAAAAAAAAAAAAAAAAAA
ATTTTTGGGGGGGGGGCCCNNNGCCCCNNNTNNCCNCCTTTTNTTTTNNNNNAAAAAAAACCCCTTTT
CCCNTNNCNTTNCCTCCANCCCGGNANTGNTTTTNCNTNAACACNGGTGTTTNTTTTNGNCGTCNNTTT
NANNNCCCCCTNTNTANNCCCCTNNTTTTTTTANNGTTTTCCTTTNCCNNCCNGNNGTGGGNACCCNTN
CCNNTNTNTTTNTNTNCTTGNGGNTTCNCNNGAGGNCCCNANCTGGGGGGGNTNCTCTTTCCCGTNCN
ANNNAANTANCTNNCTATTNNNTNAATTTCTTNNGGGTGTGNTTNGCCNCCCCNCAAAATCGTTTNCNG
GGGNNNNGNTTTTNTTTNAACCCNCNNTTNTTTTTGNNNNNGGGGGGGNNGAAAAANANATCCNCCCN
NATTATNGNCCCCCCNANNNCNCCCNCCNTTTTTTTGCANAAACNNCCCNGCTGCCT
TTGANCACAGCTGGACTCCACCGCGGTGGCGGCCGCTCTAGAACTAGTGGATCCCCCGGGCTGCAGGN
NGGAGCANAGGGNCCCACCGNCGACNNGAGGGAACTCNNTNACTGGNNCCGTGCTCATCTCAGGA
NCANANCCATTNCTTAGNGAGATAATATAGTATCAAAGCAACATGCAACCNANACCTTTTGACATTTT
AGGGCTAGTTGGTACTTNCCANTANACATGTTTGGTGGCTGGTGTCAAANGTGCGNAAAGNATTAGCT
GCCANGNNAGNGGAATGCTTGCTTTTATTAAAATGACTGTANTCTTCCTTGTTNCAGACAAGCTACAT
TGGAAACCTCCTTACAGTACAGGGNGNTCAGAAACGTTTTNAGTNCTTTGAAAGCACTATATTTATG
TTGCTCCANGGAGTTGATTTTTCTCCAGGTTTAAANAGAATNACAGCTTCTAACTTTTAATTANAANTG
NACAAGACAGAAAGCCCCGNGGANAATTAAAGCCAAANAGGATTTTTCATTNTAATTNTNGGGGGGG
AAAAAANATCCCAAGCNTTGTGGANNTAAGGNGTCNCCCCATCCAAAACNNTGTTTCCCNTTGGNTAA
AAAAANTCTTTCCTTGGAAGTTTNAAACCTTGAATNGCCCNNAAACTTGGGTNNGGNNGGGGGTGNCN
CTTAATTTANGGGGGGGGGGGGGGGNGGGTTTGGGGGGGGTTNAAAAAATNAAANNNAAGCCNTGN
GGGCCNTATTTNNCCACNNNGAAATTTACCCGNGNGGTTCCCCCAACCTTTTTAGANNGGGCNTTANN
NTTGTAAAGGGTTTTTTGCCAAANGGAAAGGGGACCNTCAAAAGGCCGGGAANCNNTTTTTTTTTTTT
NTTANGTTAANGGGGAAAANGNGCCCTTTTTGGGGANNANAAACGGGGGNCCCCAAAAAAANGGGG
GTNTTTTNGGNCNAANATTTNAACCCGGGTTNTGGNCCNNCTTTTTAGGNG
TNNANAAAGCTGGAGCTCCACCGCGGTGGCGGCCGCTCTAGAACTAGTGGATCCCCCGGGCTGCAGG
AATTCGGCACGAGGCTCACTCCTNTGGTGGANCTCANCACTCGGGAGGCCCTCGTNTNANGGACAGCT
GCTTCTCCCGGGCTGGGCTTTTCTCCCTGAGCTGCTCAGCTGGAACCAGGGACACGGTCTGAGCCCAA
CGCCAGTGTCTATTTTCCACAAAAACGGGCAATGCTGNGAGAGCCATCGGAAGACTGTCCTCTATGGC
AATGATCTCAGGGCTCAGTGGCAGGAAATCCTCAANAGGGTCACCAACCNGCCCNCTCAATGCATAA
AAAACTAGAATCTGANNATGNCTCCCAAGCTTTCCTGAAGNCTGTCGCTGAGGAGAANCCCCATGTAA
AACCCTACTTCCCANAACCATTTCGTGACTTAGAAGTNGGGAAGGGAANGGCTGCTAAAANTTGACT
TGAAAAAATGGAAGGATACCCCNGACCCCTGGAAGTCGTTCTGGGTTCAAGGAATGACCCATTCAATT
CCNGGGAGGGCCCGGCCACTTTCCANAATGGACTTACGGATGAGGGATGGGGAACCTGGCTTCTTTNA
ATCATTAATTGAANGTCTGGCGGNGAATGAATGAATGCCCAAGGACNCCCTGGCAAAGNTTGGAAAA
CAGGNCTTGGGGNAAACCCCCCTGGCNCAACCNAACNTTATTCGNGGGAAACCCNTGGGAGGGAAN
GGAAAAAGGAAAAANGGGGGANNAAAAAGGNNGGAAGGAAAAAATTGAAACCAANAGCCCNGGNG
GANAAACCAGTTTTTTAAGGGCGTTNTTACCAAAAGAACTNTTTTTTTTTTNAAAGCTCNAAAAAAAA
AAAAAAAAANAACTCGGGGGGGGGGGGCCCCGGNCCCCAAATTTNCCCTTTTNNGGGGNGNNNNNN
NNNNNAANANAGCCNNNNAAANNNGNNNNNTTNGNNNNAAANTTANNCCNNGNNTNNNNNNNANN
NNTTTTTTNGNNNNTTTNGGGANN
TTGGAAACCNCTGGAGCTCCCCGCGGTGGCGGCCGCTCTAGAACTAGTGGATCCCCCGGGCTGCAGGN
TTCCTTATTTNNNAAATNGGNTCTTAAAATAGGTTCATACTATCCTTTGNGNAGAATTGAAAACCTGCA
AGAATGTTGTTCATACATATCATGGAAATTGCACAGTAAGGGAGATTTTCATGTAAGATTTGTTTGTTCT
ANTTGCATTAATTCATAGCCATACTTGATGAATTAAGTCACAGTGGCTGACATTGATTCTGAATAAATT
GACCTAAATAAGTANGTATTAATGTACTCTNATCTAAAACTAATTTAGCATATTTGTTCTGTTTTTGAA
AGAACAGGAATTGCCCTTTAATACTTGTTTNAAATTTGAGNACCTAAACCTTCACACGAGTATAGATTC
AAATGGNCACATACATTACTTTTATAAAGNCCGTTATTATTGATTTATTTTAACCCCNAATCTGAGACA
NATNAAATATTGNTNTATTTGAAAAATGTCNTTTCTTTCCATTACTGTCATCTNACATTACCCCCATCTT
GAGGGGAANGANTCAGCNCTANGGTGNTTTNATGGATGTGAGTATTTCTCATTTTTACATAAAACTTC
ACAATTTATACTTCTCCAATCTCTTACCGAACTTTACANANTTTAAAAANNANTCNTTTTANGGCCTAN
CNANANNAACTNGNTTTTNTTNTNTGNGANNANCAGNANNTTNTTTATTNCCTTNATCCCGGCCCNTTT
TTATTTTNGACTTTTANNCCTTTANNTTNTTTTAGNNCCCCTGTNTTTTNTTTNNTATTATNTCNANNCC
NTATNTNNTNTTCTCTTTAANNNTTNTTTATANNTTTCACTTTGTTTTTATTTANTNANTCATCANNTCT
TNCNTNNTANTATNNATTNTNCTANTATCTCTNTTTCCATNCNNATANATATTNTTATNTCATNTATNN
TTTNNCTCNTTTCG
AAANCTGGAGCTCCACCGCGGTGGCGGCCGCTCTAGAACTAGTGGATCCCCCGGGCTGCAGGAATTCG
GCACGAGGATAGACTTAAACGAATACATTAACGGTGCTAAACACAGACATTTTAACCCAAGTCACGAC
ATTCTTAGCTGTAACTCAGCTCTCACGGCCTCTTGCTCACCCATGAATGGTCCCGTTTTCCTCTTGCCGC
GTGCACCTCCACCCATTGTCTTGGTGGCACATGGGTGGAACACTTGATCTGCTCGAGTCTGCCTTCAAC
ACACATTGCATCTTCAGATTTTCTACTTTTCTGTTCAAAACTAATATTCACCAAGTCAGACTTTGTGTTA
ATTTTATATCAGGGTATTGGCTGCCAGGGGGTCATCCCTAAGTGGCCTGAAGATGGACAAAGGGAAGT
AACAGGCACGTGATGTTGGCAAGGATGCTTCTAGGGCTAGAGGATCAGTGGTGGGAGAGAGCTGCAG
AATCCACCAGCCAGAACTGCAGATAACGAATCTTATGGTCAGGGGCTGTGACTGAGAGAAGGAAACT
GAGGTTGTGTTCTGAAAGTACATAAACTCTCACATATACCCAGTTCTTCACCATCTCCCCTCCTCACTTT
GCAGTGCCATTTCTTTTTGCATTANGCAATTTGCTCAGACTTTCCAAAGCCATGGCCCATCCGTTCTCTG
GAATCCCCCACACCTTCTGAGANGTGGATCACCACGTCCTGCANGGCTGCTCCCTTCCAAACTACCTTT
AAGAAGATGCAGGACAGGGGAGCTGTTTCAACCAGAAAGACCAAAATCAANNCCGAGGAGCCAAAC
GTGGTAAAACAGAAAAANGGCAGGGGGCNAAATGGTTTNCTTTTGGGNTTCNGGTTTCTTTTTTTCCA
TCTGGGATGGGTGGCCCCANANANTNTNTTTANTCGCNTAACATGGTNCTTCTTTTTTTCCCTTCCCAANT
TTTTTTTTTTTTTAATNAAAAA
NCNNNATCTGGAGCTCCANCNCNGTGGCGGCCCGCTCTAGAACTAGTGGATCCCCCGGGCTTGCAGGA
AATTCGGGCNACGAGGGTCATGCAGGCCCTGCTGGGCCCGCAGTCCCTGCCTCGGACCAAGGAGGAG
AAGCACAANCCAGCCCCCCAGGAGGACTCGGTANCTGCCAGCAAGCAAGCTTCCCCAGCCCCCAACA
TCAGCAGCGGCTGCCAGACCCCCCGGCGGAAAAAGNCGCCGTGATCTGTCAGCCACATGTCCTTAGAG
GGTCAGGGTCGCGCTTGCCTCTGGCTCTCTGCCACAGGAGGGCCCAGCCGCCGTGCCTTAGCCAGCTG
CCATGCATACGCANGTCAGGGNGCTGGCCTGTGGGGGGCGCCAGCCCTTCTGCTTCAAGGGCATCCCC
CCACCCCCACCACGTCCGGTCCCAGGACACCCTCTGACCTGCTCGACGGCCCAGGAGGCCCATGTGTG
ACCTGGCAATCGGGGACCCTTGCTCTNTGCGCCGACGGCAGGGGTCCGAGTCTGAGCCCCAACCCCGG
CTCCTGCACACGGGGTGGAGGTAGGGTGGNATGAAGCTGACCTTGCTTGGGGAGAGGAGGGAANACC
CCCANGGTCTNCTCTACAGCANCCTGTCTGNGGTCCGNATCCGTGAACCAATAAAGGCAATTGTGNCT
TGACCCNNNNTGNNTNNANANNAANNAATTTGGGGGGGGGGCCCCNAACCCAAATTNNCCNTNNANG
GGNNNNNNNNNTNACNNNNNNNNGGGNNNNANNNNNNNTNNNNNNNNNNNNNNNNNNNNNNNNNN
NNNNNNNNNNNNNNNAANNNANNNNNNTNNNNNNNNNNNNNNNNNNNNNNNNNNNNNNNNNNNN
NNNNNNNNNNNNNNNNNNNNNNNNNNNNNNNNNNNNNGNNNNNNNNNNNNNNNNNNNNNNNNNNN
NNNNNNNNNNNNNNNNNNTNNNNNNNNNNNNNNNNNANNNNNNNTNNNNNNNNNNNNNNNNNNNN
NNNNNCCN
GNCCTATAGNGGANGNCGNATTTNCAAGGGCGGCCGCTCTAGAACTAGTGGATCCCCCGGGCTGCAGG
ANNNCGCCATNATGNANTTTTGNTGCCCTNAACCTTCAACGAGGNTCNCGGGAANNTGANNNACATA
NTGTNTANNAAATTTCAANANNANCNGATCNTGGATGAANGANATTANCGCAGGANCNTCCANTGGT
NNNCTAANTGGANNCNCAAACTGGTNCNAAANNTNTAANNCNTNNNNAAANCCGATAACNCCNATAT
TTNTTNNTACTTCTGNNNAGNATATCTNCCCNNNACNNNATAGCAAGTCNNNGCGNANTNTGANGNTN
GACTCTTNNNNANAANANNGANNANACCATCCNNCATTNNTNATCNANGNAAGGAANNANANGGCN
NATCANNTTNAGNGNACNNCCNCNTTTNTANTCNTNGTATTNNTNGANTNAANNANTNNCNNANNA
ANTCNANNNNACNAAGGNAGAAAAGTNCTNTTNNNNTNNAATAANATNANAGNNTACNNTGTNNTN
ATCATTCGNGCTGCNCNNNANNNNNANAGNTATNTGNNNNTNTNNTNGANTGNNNNNGGGNAAGNN
NTANANTNNACTCACNGTAAAGNANNCNNNTCNNTCTCCTAGCANCNTTTCGTNACCAANNCCTCCTC
NCAAAAGNAGNATNTAANGANATGATGCNAANGCTCNCCAAACNNNATAACATNNTAANCNNCNNA
NANTTNATCANTANNCATNTNNANGTNNGNNNNCCACATTCCCATATCATATACCANNNCTCNGN
CCTGNCCACGANGTTTNTNATANCAATNTNGNNNNCCANNTGNNNNCNTNTNTNTNATNCANNNATN
NAAAGCCNNTTCGGNTCACNANNTNTTTCTTNTNTCNAANTNNCNNCTTANNNCANAACTAANNCCTA
TNNAGTNNNTNNTCNNATNNTGNNTCCNANACNGNCGANCTNNNGNCCACGNTNNGANACGGANCNC
GNNTCATANCCATCANGAANCNACGTCNATATCGNCCTCCNTCAANTNGACATNNNNACGTNNNCTTT
CAGNTGNCTCGNTNCNCNNATGCGNGNTACTANAGANTTNNNNGNCGNTNGNCG
TTNAGNNTATCTGGAGCTNCACCNCNGTGGCGGCCCGCTCTAGAACTAAGTGGGATCCCCCGGCCTGN
AGGTCNAGTTNTTTTTTTTTTTTTTTTTTTTTTTTTTTTTTTTNAAAAANNNNNTTTTTTTNNTTTTNAAA
NCNNNNCCNTTTTTTTNNNNNNGGGGGNNCNGNNNNTCCCCCCCCCCCCCCCCCCNGGNNNNCNNNN
NNNCCNAAAAAANNNNNNNGGCNNTTAAAAANCCCNNCNAAANNGGGCCCCGGGGGGGGGNNNTNNN
TTTNNGGGGGGNNNAAAAATTTTTTTTTTAAAAAAAAAAAAANNNTTTNCCCCCCCCCCCCCAANTT
TTTTNNGGNGNCCCCCGGGGGNAAAAAAAANNCNNTNNGNNTNNAAAAAAANNNNNGGGGGNTTNN
NNTNCCCCCCCCCNTTTNNGGGGNNGGGGGNNTNNTTTTTTTNGGGGCCCNNAAAAAAACCCCCCCNT
TTTTTTNCNNAAAAANCAANNGGGNTTNTTTTNGAAAAAACCCCCCCNNTTTTTANTCCCTNGNGGGG
CCANAAAANGGGGTTTNNNNNAAANATTNGGCGGGGNTTTTNTTTTNNANNCTTTTTTCCCCCCCCCC
AAAAAAAAAAAANGGGGGGNNTTTTTTTTTNGGGGGGAAAAAAANGNCCANAAAAANNCCCCCCTTG
GGNGGGGGTTTTTTCNCTGGGGGANATTTTTTTTNTCCNCCCCCCAAGATTTTTTCTNAANTTTTGGGG
GGGTTNTTNNNNGGNGAANNAAAAAAAAANTTTTTTTGGGTTNTAAAAAAAAAAAAACCCCCCCCCC
CCCCCNNTTTTTTNGGGGGNCCCNAAAAACNAAAGGGGGGGGGGGCCCCCCTTTNANACNCCCCCC
GGGGGGGGGGTTTTTTTCNCCANGNGGGGGGGGGGGNNCGCCNCCNTNNTTTTNTTTTTTTTTTGNGG
GGGTNTNTAANAAAANNNNNCCN
TNAGNNTATCTGGAGCTNCACCNCNGTGGCGGCCCGCTCTAGAACTAAGTGGGATGCCGCGGCCTGN
AGGTCNAGTTNTTTTTTTTTTTTTTTTTTTTTTTTTTTTTTTTNAAAAANNNNNTTTTTTTNNTTTTNAAA
NCNNNNCCNTTTTTTTNNNNNNGGGGGNNCNGNNNNTCCCCCCCCCCCCCCCCCCNGGNNNNCNNNN
NNNCCNAAAAAANNNNNNNGGCNNTTAAAAANCCCNNCNAAANNGGGCCCCGGGGGGGGNNNTNNN
TTTNNGGGGGGNNNAAAAATTTTTTTTTTAAAAAAAAAAAAAANNNTTTNCCCCCCGCCGCCCAANTT
TTTTNNGGNGNCCCCCGGGGGNAAAAAAAANNCNNTNNGNNTNNAAAAAAANNNNNGGGGGNTTNN
NNTNCCCCCCCCCNTTNNGGGGNNGGGGGNNTNNTTTTTTTNGGGGCCCNNAAAAAAAGCCCCCCNT
TTTTTTNCNNAAAAANCAANNGGGNTTNTTTTNGAAAAAACCCCCCCNTTTTTANTCCCTNGNGGGG
CCANAAAANGGGGTTTNNNNNAAANATTNGGCGGGGNTTTTNTTTTNNANNCTTTTTTCCCCCCCCCC
AAAAAAAAAAAANGGGGGGNNTTTTTTTTTNGGGGGGAAAAAAANGNCCANAAAAANNCCCCCCTTG
GGNGGGGGTTTTTTCNCTGGGGGANATTTTTTTTNTCCNCCCCCCAAGATTTTTTCTNAANTTTTGGGG
GGGTTNTTNNNNGGNGAANNAAAAAAAAANTTTTTTTGGGTTNTAAAAAAAAAAAAACCCCCCCCCC
CCCCCNNTTTTTTNGGGGGNCCCNAAAAACNAAAGGGGGGGGGGGCCCCCCNTTTANACNCCCCCC
GGGGGGGGGGTTTTTTTCNCCANGNGGGGGGGGGGGNNCGCCNCCNTNNTTTTNTTTTTTTTTTGNGG
GGGTNTNTAANAAAANNNNNCCN
TTNNNNAAAAGCTGGAGCTCCACCGCGGTGGCGGCCGCTCTAGAACTAGTGGATCCCCCGGGGTGGAG
GCAGGATCAAGCGATTCCTGGCCAAGAAACAAAAGCAGAATCGTCCCATTCCTCAATGGATTCGAATG
AAAACTGGCAATAAAATCAGGTACAACTCCAAGAGAAGACATTGGAGAAGAACCAAGCTGGGTCTAT
AAGAAGCAAGCTGGGTCTATGAGAAGTGGTCTTAACATGTAGACCACTTTTTTAAGCAGCGAGATCAC
AATGAAAACATCACTACTGTAATGCTTGGCCCATGATGTTATTTCCTCACTATCAGTCTGAGACCCAGC
AATAAATATAAAACGTTGCAAAAAAAAAAAAAAAAAANNTGGGGGGGGGCCCGGNNCCCAATTCCCCC
NTNNNGGGNNCCNNNTANAANNNGANNNTANCATTATTGGNTGCNNNCTGATAAACNNAACCATTTG
CAAACNTGCCCTTANNGGGACNTTGNTTTGNTCTTTGANGNCNTTNACCTTTGNNGACCCTNACCCCTN
CAACCANCATNTNATNCATTACCTNCCANNCCCANTTTNCTTTTTGGNAAAANTANNGNGGCCTGGNN
NNTGGGANCCCNNCCCTGGGCTNAAGNGNNANNNNTGNTNTTGNNNNNNNGNNCANNNGGNNANAA
AAANTACCTNTNCNNNCTTNNNNNTGNNNCNTTTTNNNAAANCNNNNNNNNNANNNGNNNCCTANNG
NNNTTNGGNGGGNTNNNNNNNNNNTTCNNGNGNANGGGCCNNNNNNNNGGGNTNGGNNNNNNNAA
NNNCCTTNGNNNANNGGNAANGNNNTTTNNNNNGNNCNANGGNNNGGGGGGGGGNCCCNNNNNNCC
NGGGGGNNNNNNNNNAANNNNCCNNNNNCNTTTNNGGGGGNTTNTNNNNCCCNTNNCCNNGGGG
TTGAAAAANCTGGACTCCACCGCGGTGGCGGCCGCTCTAGAACTAGTGGATCCCCGGGCTGCAGGNA
NNTGCTGNNACGCTCNGNCCNCTNGGNTATGTTNGCTCTCNCGNNGNNGNTGGANTAANTGNNNTTNC
NTGNCCCNTCNTGCCCTTTGGTCCTGAGGTGNTACTGTGGANNCTGGNCCTGNTGNTNCTCCCNGCCC
TACTGNANNNNTTGGACANCNAGGTCNTNNCTGNGNNNGCTATNANTNGACTNTCCTGNCNNACGCA
NATCNAAAGAANGATCACGATGGAGGCCNCNACNANNGNACTNANGATGCCNCTGAGNGCCGAGAC
CGTNANCTCNNGGTGGCNCCCCNCTCNTAACCGTNNGCTGNCNGNTNGAAAACANCCGNAGCCCNNA
ANGGNNTTTTCNAANAACNCTGCCGATNATGACGTGACGNAAGNATGTGCCCTTTGATTGGCNTGAAC
CGNTNATGTTGCTGGACCCTAANAANGGTTGGATCANGATGCCTNNNGGTTTTNGGAACCTGGCAACC
CGAGANAACTTTGGATTACNCANNGAANCCATTTTTGACCAATAANAAATGGTTTTTTGGGGNNNNAA
ACNCCAAGGAAAAAAAGGCAGGGNTGGTANCCNGNNNTNGNATTAAACCCCCTTTTTCCAANTTTAN
GTTTNTTGGGGGGANACGGTTTCCGAAACTTGGCAAAGGGNNANCGTTTCAAAATTAATTTTTCTTGG
ACACTNGAANTCCAACCCCNGGGCTTTANAAAAANTTTTTACTTTTCTATTTGCAAAAAAAAATTTTTT
TTTTTAAATTGGGCNNAAAAAAAAATNGGNANCCNTTCNGGANGGGGGCCTTCTTCCCCCAAGGGTTT
CCCATTNNAAATGGGGGNTTTCGGCCCNGGGGGNNAAAANNNCGGTTTTACCTTAAAGGNGNGNACC
TTNAAAANGGGTNGCCNAAATCCCCCCGGGGACCT
TNNNNNAAAAGCTGGAGCTCCACCGCGGTGGCGGCCGCTCTAGAACTAGTGGATCCCCCGGGCTGCA
GGAATTCGGCACGAGGCTTCGCTGGGGTCTGAGCCGAGCCGAGGCCTGGCTGCTGCCGCCGCCCACAA
GCTGCTGTCACCGGGCTCTGCACAGACAGGTAGAGGGCACCGAATTCCGGAGCGCCTACAGCCTGGAC
AAGCTGTACCCGGAATCGCGGGGCGCGGACACCGCCTGGAGGGTCCCGGGCGACGCAAAGCAAGGCA
ACGATGACATTCCTGTAGATCGCTTGACGATATCTTACTGTCGGAGCAGTGGTCCTGGGGGCCAGAAC
GCAACAAAGTGAACTCCAAGGCTGAAGTCAGGTTCCACCTGGCAAGCGCCGACTGGATCGCAGAGC
CCGTGCGGCANAAGATGGCTCTCACGCACAAAAATAAGATCAACAGGGCGGGAGAGCTGATCCTCAC
GTCTGAATACAGCCGCTATCAGTTCCGAAACCTGGCAGATTGCCTCCAGAAAATTCGAGACATGATTG
CCGAGGCCAGCCAGCCAGCCACAGAGCCATCCAAAGAAGATGCTGCACTTTAAAAACTCAGGATAGA
AAACATGAATCGCGAAAGGCTCCGAAAAAAAANAATAAACTTTTGCCATCAAAAACNAGCANGGAGG
GTCGGTACCGGACTTGAAAGCACCCCCTTNGAGCCAGCANAANCCCCTTCANGGGCANCCCANGTNG
GCTCGGGACACCCAGGAAAAACGGGNGTCTGTTCGGGNCANCTGGTTCGGNNTTTTACGTNGGGAAC
CCGTNNNAAANNAAGGNNCCNTTCGGGAAAACGNAAGGGTTTTGGCCCCTTGGTCCCCGCCAGGG
NNTTTTTAAAGGGGGNAATNNCCGGGTTTTTNNCCNCCCTTTGGGTAAANCCCANGGNCCCGGGNTTA
AANNGGCCATTAAAAATTNTTTAAACCCCNAAAAAAAAAAAAAAAAAAAAAAN
TTGANCAACAGCTGGAGCTCCACCGCGGTGGCGGCCGCTCTAGAACTAGTGGATCCCCCGGGCTGCAG
GCTTGATTTTTAGAAGAAAACAGCCAGACTGAATACCACCCAGTGACACCAGNGCAGGTAATAAGCA
CCACCCATGTTGCACGTCAGGCACACTTAATCCTCTACCCTACCAGGAAGATCGAAGAGTAATTTGTTC
TGGAAAATAACTATTATCTTGTCTGTTAGTTCATGTATCATAAATCCTATAACTGTATGTGGCTTTGCTG
CTTATTCTTACACTTCATCAGGTCCCAAAGACATATTTCCTCAGCACTGAGAGTGTTACAGGTACAGT
GTATTGTACCTGTAACAATCTTCACAGGTTTTTTGCTGTATATAGGATACGGTCTTTTTGCTGTATATAG
GATACAGTCAGATTATTTGATTCTATATACATTTTAATTCAGTTCAAAGGACCTTTTAAGACAGCACCA
TTTTTCTCACAATATAGACTTTGCTGGTTTCCAGCACATGAAATACATCTCCTGACAGAATATTTTAAT
GACACATGATAGGGTGTCAGTCAGCTGATCTGTCATTAGCTTGTATATTTCTGCCCGAGACCAGTCCTT
AGTATTCCAGTACTGATTTCAGGCAGCATGTCTTCTGAATGAATATCCTCAGCAGCCTACTTGNTAGCA
TGTATTTATTGNCATCTGGTTANAAAACAGTGAGCCCCAAGNGATTACCGGNCCTTGNTTCAGTCCCA
CTTATTCAGGAAANGGGAATNCCANAATCATGGATCCCCCAAAAAAACCTCGCCCCTTCCANNGAAAC
CGACGGGAATGGGAAATGTGCCCTCCANAAANTTTTTGGGGAAAANGGGCTTGGCAATCAAATCAAC
NTGGNACTGG
NNCCNTCTANNTCGTAAAAAATNNTNCCCTCNNCCNCCNCCCCCCCAACCGCTCCNCCAACTCATCTT
ACCNACCTCCCCANNGNNNNNNNNTNNNGTNCTCACTNCCCTTGAGNNNCAACTGGACTCCACGCGN
GGGCGNCCGCTCTACAACTAGTGGATCCCNCNNGCTGCNGNTAATTCNGACNCGAGGGNTANACATN
CTGCCTCCCCCTACTCACNTNNCTNTTNTAGGCCTTATTACGGACNAANGGTTANTTACTCNNCTTTCC
TNTNTCNTTNTCACATCGNNNNACNNTNNNATTNTAANANTANTNTTTANATACCTNNTNAACTANAA
TCCATCCNCNTTNAATNNTNTNTNGNCAAAATATNCATTNNTANACNTANTTGTCNATGNTTNTNTAA
ATCNNNTTTTACTTNNATTNNNCNTNTNAAANNNTTTNNTNNACNCTACTTNNATGNANTTTTNNANN
NTTNNTNTTANNTTCNTTATATNNTTTTATNTGGNTNTNTTCNNATCNNAACTATNATNTNNTCTTN
NNTANAANTACANAGACANTCNTTCTTATTTACCTACTNTTTTTCTCNCCCATACNTTNANTCTTCCTTC
ATNTAANNCTACANCNTNCAAATTTTTATCATANNTNNNTCTNTNNNCNTCCNGTNTTATCATNNNTTN
CCNGCTTACNTANCTCTNNCANTNNTCTTATTNTTTTNTNANNTTTTNNNCANCTNNGCTACNCCTAAT
CTCCCTNTNNTNAANGNTTTCAAACNANNTTNATANTTNATTCCCANTATANNCTACTTTNACAANCN
NNTCCTATTNTNNTNATNTNNTCAATNNTCNTTNTTCTATCNNTAATNNANANNNNTAACNANCCCN
TTNANTTCCATNATTTTTTNAAANNAANNTANNTCAATNGANNATCNCGTTTTTATTCCGTCNNCTNNT
TACGNCTANTTCTTNNTANANTANNTTTNTCTTTTTTTCTATNNGTACNNTTNNACTCNNCTACNTACN
CNNTTNNTCTGNTCNATCTNTAANATATCTCNCATATACTNTGTNNNACTNNNTCCTCNTATCTTATAT
TANCTGANTTACNTCACTTTCNTCAGNNNTTNCATNTATNGTTCCAACNTACTNTNTATNTGATANTCT
NTNCACTCACANNTCACNNTCTNTCACANTTCATATTTATTACAATATAGNNCACNNATACNNNNANTC
NCTNNCATNCTTACATTTNTGTTCNNCNTATNATTCTNNNNCATNNNATTNTGAGTNNTTATNTATCTT
CNNTANTCTCNCNCGACNTANTNTNNTCTTAACTTTATTTNNACTNTNNATATATNTNNTNTCANGATT
NACTANNNCNTNTTTTATATATAANGATACTANTTNGTCCTATNACTTCNTCTNTATANCTTTACACTN
NCTNNCNTCATAACNTNNTNNTTCTTNTCTNTTCTCAATCGNTCCGACNNTCNACCCANCTNTTCACTC
NNATATCGNCTGACNCTGCANGTTTACTCTNNATCCTCTACC
AAANCTGGAGCTCCCCGCGGGGGCGGCCGCTCTAGAACTAGTGGATCCCCCGGGCTGCAGGCTTTTTC
ACTTTGCTGAGGCCAAGTTCAGAGCAGGGGACTTCAGCACCACAGTTATCCAAAATGTAAATAAGGCC
CAAGTGAAGATTAGAGCAAAGAAAGATAATGTAGCAGGTGTTACATTGCCAGTATTTGAACATTATCA
TGAAGGAACTGACAGTTATGAACTGACTGGTTTAGCCAGAGGTGGGGAACAGTTGGCTAAACTGAAG
AGGAATTATGCCAAAGCAGTGGAACTACTGGTGGAACTGGCTTCACTGCAGACGTCCTTTGTTACTTT
GGATGAAGCTATTAAGATAACCAACAGGCGTGTAAACGCTNTTGAACATGTCATCATTCCCGGGATTG
AACGTACCCTTGCTTATATCATCACAGAGCTGGATGAAAGAGAGCGAGAGGAGTTCTACAGATTAAAG
AAAATACAGGAGAAGAAAAAGATTCTCAAGGAGAAGTCTGATAAGGACTTGGAGCAAGGGAGGGCA
GCCCGGAGAGGTGATAGAGCCTGCTAATCTCCTGGCTGAAGAGAAGGATGAAGATCTTCTGTTTGAAT
AAGCTTTCCTGCTCTGGTTCTTTCAGAAGGCCTAACATGTCACCATTTTAATTCATGGGTGTGTANGGT
TTGTTGNATGGGTATTTATTTTTTGGCTTAAGAATTCCCCCAGTTGNAAAATTTCCCCTGAATGTCTTGT
TTATGGGATCTACGTTTGCANAAATCATAATTAANCAACAATTTATCCCCCAATGAAANTTTGTAGAA
CCTGTCCCCAAACCTGTAAAATTTTTGCCTGNNNNAAGGGNCNCCCTTCTTTNANTTTTTACTTCNGNN
ACAAGGGGTTTTGNTTTTCCCAAANATANAAAANAAACCCCTTTTTNGGAAGCANTNNTNCTGGCCNNT
TNNTCCCAAANNCCCTTTGNTNCCNCCGGCCCCCN
NCNNNATCTGGAGCTCCACCGCNGGGGCGGCCGCTCTAGAACTAGTGGATCCCCCGGGCTGCAGGAN
CCCNCCNTTCNNACCGAGGNGCCCCTNNNNNCCNACGNCNNCCGNNANAANGTGACCCACATNATGT
TCNAAACTNTCNNTGNCNCAACCGTGTATGTGGANATTCAGGCGGAGCTNCCCCTGNACGCCNCTGGA
CNCACAACTGNCNTNGTGCTNNACTNTGGAGANGGCNTNACCCACGNNGGGCCCANNCATGAGGGNT
ACNNCCTGCCCCACCCCATTTNTNCGNNTGGACCTGGNTGNNCAAGATATCANCGANTACCTCATNAAG
ANANTGACCGAACAAGGCTNTTACTTCTTGACCNCCGNTGACCCTGACATTGTCCNTATACATCNNNG
NGAAACTGTGCTTCGTAGNCATGTACTCTGANAATGATATGGTNACTGNCGNATCTCTNCTCCTCCCTG
AAAAAGANCTATGATCTGCCTGATNGGCAAGCGATCACCATCANGAATGANCGTNCNCGCTGCNCTG
ANACCCTGNTCCAGACCTCNCTCATAGNNATGGNNNTATGNTGNCATACTACGATACGACCCNACAAC
NTAATTCATGANGTGTGAANATNGGCTATACAAANANGNGACGCTCTTATGCTNAACAATCGTANCTT
CTTNNCNNGGGTACCACCATGGTACCNCATNANCTTTNNTNGTCCGCAATGCCTTNNGGAANANCACG
GCCCATGGGNCCCCNCGGNCNATGANGNATCTNGGNATNATNCCCCCCTTCCCGAGCCNCCAAGNTNC
TTCCTGTCNNGAATTTGNTNGGATTGCCATACCNAGGNTCNTTCTTTGNNNCACCCTTTCCCNCCAAAN
TTGNGGGAACCCCCNAANCAGGAAGTTTCNAAGGGAAGNCCCGGNCCCNTTCCTTNTTNCCNCCCGNT
NAATGGGTTTTTTCCCCNCACCCCCCCCTCCCNCC
GCCTATCTGGAGCTNCACCNCNGGNGGCGGCCCGCTCTAGAACTAGGTGGGATCCCCCGGNCTGCAGN
ANTTCGGCACGAGGCGACGGCGGCAGCGGGCGNAACGGGACTGGAGGNACCANCGGAANGNAGGNG
GNTNCTGCAGCCGNCCCNANNGNNANGNCCTCNGAAGGNNCNGNNANGGTTNAANCCNTTNTTNTTC
CCNTNNNNTTTNTAACNNTNTAAANCNNTGGANTNCCNTNCCCCNNCNNGAAGGGCNGGCNANNCCA
GNGAATGGGGCNTTNAANNGGAANCCAGAAGGCCCAAATATTGAAGGGAAGAACNTCTTTCAACNGC
TGTCCCCACCNTGGAAGAANAATTATCATTGACACAGGAAATTACACCCCGGAGGAGCNCTNTAN
ACACGTNTTNACAGGAGCTCTNGNNTCTGNGTTGGNATACNNCANGGGGGCCGCCTGANCCCTCNGA
CATANACNTGCATATACANGTGTGCGAGATAAATTNAGCCCTCTGTTTCTTATATTGAACCCTATACC
ACCCACACANATATTTTGTCAGNGTCNTTGAGACCGCCNTATATATAATCAANGGNGAGCCNCCATGC
TGCTGGCTGATNTGANCTACNCTCTGNNCACCAAAGAAGNGGCACGCCTNTGTTGGTGGACCACNTNG
TCCCGNATGANAACAACANGCAGGGGGAGAAAAACTACCCTGGTTAAGGNNGGGTTGCATGGNGCTT
TCCTTCCAAAAAAAAGGGGGGGGGGNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNCT
GCCTATCTGGAGCTNCACCNCNGGNGGCGGCCCGCTCTAGAACTAGGTGGGATCCCCCGGNCTGCAGN
ANTTCGGCACGAGGCGACGGCGGCAGCGGGCGNAACGGGACTGGAGGNACCANCGGAANGNAGGNG
GNTNCTGCAGCCGNCCCNANNGNNANGNCCTCNGAAGGNNCNGNNANGGTTNAANCCNTTNTTNTTC
CCNTNNNNTTNTAACNNTNTAAANCNNTGGANTNCCNTNCCCCNNCNNGAAGGGCNGGCNANNCCA
GNGAATGGGGCNTTNAANNGGAANCCAGAAGGCCCAAATATTGAAGGGAAGAACNTCTTTCAACNGC
TGTCCCCACCNTGGAAGAANAATTATCATTGACACAGGAAATTATTACACCCCGGAGGAGCNCTNTAN
ACACGTNTTNACAGGAGCTCTNGNNTCTGNGTTGGNATACNNCANGGGGGCCGCCTGANCCCTCNGA
CATANACNTGCATATACANGTGTGCGAGATAATTNAGAGCCCTCTGTTTCTTATATTGAACCCTATACC
ACCCACACANATATTTTGTCAGNGTCNTTGAGACCGCCNTATATATAATCAANGGNGAGCCNCCATGC
TGCTGGCTGATNTGANCTACNCTCTGNNCACCAAAGAAGNGGCACGCCTNTGTTGGTGGACCACNTNG
TCCCGNATGANAACAACANGCAGGGGGAGAAAAACTACCCTGGTTAAGGNNGGGTTGCATGGNGCTT
TCCTTCCAAAAAAAAGGGGGGGGGGNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNCT
TTTNANNACNNTCTGGAGCTCCACCGCGGGTGGCGGCCGCTCTAGAACTAGTGGATCCCCCGGGCTGCA
GGNNTNNATCCNTGCNCATCCNAAAGGATGATGAAGTTCAGGTTGTACGANGGCNCTACAANGGGCA
GCAAATTGGCAAANTAGTCCAGGTTTACAGGAAGAAAATACGTTNNTCTACATTGAACGAGTGCATCT
GGGAGAAGGCTAATGGCACAACTGTCCACGTGGGCATTCACCCCAGCAAGGTGGTTATCACCANACTA
AAACTGGACAAAGACCGCAAAAAGATGCTCGAACGTAAAGCCAAATCTCGCCAAGTAGGAAAGGAAA
AGGGCANATATAANGAANANACANTTGAGAANATGCATGGAATAAAGTCATCTTGTCTACAGCTTTC
ATTAAAAACTGTTAAAATGAAAAAAAAAAAAAAAAANNTGGGGGGGGGGCCGGGACCCNNTTTGCCCT
TATNGGGNGTGANAAAGNTNNCGATGGTGGCCGNTTCTACCGGGCTGATGATGCCAATGTGGNCCGT
GACCGTGACCTTNAGGTGGACACCACCCTTCAAAANCCCTGANCCAGNNGATCGAGAACATCCNGAG
CCCTTGAAGGCNNNCGNAAGAACCCCGCCCCCNCCTTNCCGTGACCTCAANANGTTNCCCCNTTTGAC
TGGAAAAAAGGGANAAATACCTGGNATTGACCCCCAACCCANGGNTTGCNANCCTGGATTNCCCATTT
AAGGNTNTTGGNAANNATGGGAAACCCGGGNGAAAACCTTTGTTTTTCCCCCCCCCTTCNNCCCANN
NGNGGGCCCAAAAAAAAACNTGGTTTTTTNANCCNNGAAACCCCCNANGGNAAAAAAAGGCCCNTTN
TGGGNACCGGGNNAGAGNCNTNNACCCGNCGGNATTTNCANTTTNNNNTTTTGNNGGNCCNNGGGGT
CCCCNANCCTTCCCNTTTTNGNCCNTCCTNNNTNNANTTTTCCTTGNCC
TTNAANAAAACTGGACTCCACCGCGGTGGCGGCCGCTCTAGAACTAGTGGATCCCCGGGGTGCAGGA
ATTCGGCACGAGGCAGGCAGTCGTCAGTCAGGTGCTATTGTGTCCTGGGGGTGGGAGGGCCTCCTGAG
GCTCTGCCTGCCTTCTCTTCAGCCCCACTGAGCGTCAGCCTCGGGGTCTCCTGAGTCAGAACCCTCCCT
GCTGTCTCTGGTGGTCACTCAGGGCTTGCAGGTGACCACAGGGCCTCCCTCGANAGGAGGGGCTGCCG
ATGGNATCTCCACACATAGGCACTGGCGGTTGTTCCGCTTGTCCACCGACCTTACTCCNTGCCCGTCGT
CTGTCTCGGCCTGTCTGGGAATCGCCAACAANAGCGGCTTCTCACGGGCCCTTGGTGTGGTGGTGNCC
GCCTTGGGGTTCNTTTTTTTCAAANTAAGANGCGAACCTGCANNCAGTAACCCTTGCACTTCCTGCAAG
GACAACAAANNGNGAAGTCACTGNGAACAAGCNTTNTTAAAAAAAAATCTGAAAATAGTCCTAANTG
GCCAAAATGGTTTAAACGAAAAAAACATTTGACCTTTGGGAAAGAAAGGGAACCGGCNCCAAAAGGT
AGGGGGGGGCCATTCCGGATCCANGGGGTCAAAANGGCANTTGGCACAACCGGCCCAATGTGGCCTG
GTTAANCCCATGGAATNTGGGGTTGAANCCTAANTTCCCCCCCTTACANTTTTACANAACCAAAAAAA
AAANCCTTTTAAAAAAAGNCCCTTTTGGGCNTANATTCNCTNGAATTNCNTNGGGGCANTTTGGNNAG
CCAANTGGAAAGTTTTTTTTNAAAACCNTTTCCTTAAAAATAAAAAAGNNNNGGGNAAAATTTTTAAC
TTTTAAAGGCNGGGNGGGGNCCAAAAAAACCTTTTGGNNNNAAAAAACTTTAAAAGGAACCNTTTGG
TTTTAACCTTTGGNAANGGNNNGGGGNGGGNAAAAAAAACCCCNNNTT
TTNNNNAAAAGCTGGAGCTCCACCGCGGTGGCGGCCGCTCTAGAACTAGTGGATCCCCCGGGCTGCAG
GAATTCGGCACGAGGCTGCAGGCAGGTTGTTGGGTTTCGAGGCCAACGGGGCCAACGGGTCTAAAGC
AGTTGCAAGAACAGCAAGGAAAAGGAAGCCTTCTCCTGAGCCAGAAGGTGAAGTTGGGCCTCCTAAG
ATCAATGGAGAGGCCCAGCCGTGGCTGTCTACATCCACAGAAGGGCTCAAGATCCCCATTACTCCTAC
ATCCTCTTTTGTGTCGCCACCACCACCCACTGCCTCACCTCATTCCAACCGGACCACACCGCCTGAAGC
GGCCCAGAACGGCCAGTCCCCCATGGCAGCCCTGATTTTAGTAGCAGACAATGCAGGGGGCAGTCATG
CCTCGAAAGATGCCAACCAGGTGCACTCCACCACCAGACGGAACAGCAGCAGTCCACCCTCTCCGTCC
TCTATGAACCAAAGAAGGCTGGGCCCCCGAGAGGTGGGGGGCCAGGGGGCAGGCAGTGCCGGAGGA
CTGGAGCCAGTGCACCCCGCCAGCCTCCCGGACTCCTTTNTGGCCGCCAGCGGCCCACTGTGCTTGCAC
CCTNTGCCATGAACCGGNTGGAGGACACCCACTTTTGTGCAGTGGCCCTTCTGTTCCTTCGCACAAGTT
NTGCTTTCCCTTGCTCCAAAACAAAGCATTAAAACAGCANGGGAACTAAGTNGGAGAGGTCTATTGGT
CCCAGTGGGGGAAAAAATGCCCTNTTTGNGGGCCTCCAAATGTCCCCCTGGGNCCTTTATTGCAANGG
GGAAAATGCAANCCTTTCNTTNGNTTGGANAATTTGAAAGTGNAAAAAAANAAAAAAANACTTCGG
GAANTTTTCGGGTTCAAAAAAAANCCCCATGGANTAAATCTTTAACTTAAAACTGGGTTNGAATTGGA
TTTTATATTTTCCCCCTTTTTTTTTTTTTNTNC
TTNNNCAAAACTGGAGCTCCACCGCGGTGGCGGCCGCTCTAGAACTAGTGGATCCCCCGGGCTGCAGG
CGAACAGTCCTCACGCCGATCTCTTTCCTGGTTCCCACCCGCGCCAGGTGACNCGGAGAGCTGACACC
ATGGTTNATNAGGNGCTCTACCGGNCGCTGGTCTCCACCAAGTGGNTGNCGGAGTNCGTCCGGGCTGG
NAAGGTGGGCCCTGGCCTTCGGGTGCTGGATGCGTNCTGGTAGTCGCCGGGCACCCGCGAGGCCCGCA
AGGAATACCTGGAGCGCCATGTGGCCGGCGCCTCCTTTTTTGACATANAGGAGTGTCGGGACAAGGCC
TCGCCTTACGAGGTGATGCTGCCCAGCGAGGCGGGCTTCGCCGACTACATGGGCAGCCTGGGCATCAT
CAACGACACGCATGTGGTGGTGTACGATGGTGACGACCTGGGCAGCTTCTATGCACCGCGGGTCTGGT
GGATGTTCCGTGTGTTTGGCCACCGNACCGNGTCCGTGCTCAATGNTGGCTTTCCGGAACTGGCTGAA
NGAGGGCCACCCCGGTGACATCTGAGCCCTCACGCCCAAAAGCCAGCCATCTTCAAAAGCCACGCTGA
ACCCGCTCCCTTGCTCAAAGACCTACGAACANGTGCTGGAGAACCTCGAATGAAAGAAGGTTTGAACT
TGGTGGAATTCACGGGCCCANGGGCCGGTACCTTGGCCACACAAGCCCGGAAGCCAAAATGCAATAA
GGAACTTGGACTTCGGGCCACAATNCCAAGGCTTNGGNCNAACAATGCCCTTTTATTGAACTTTCCTTA
ACGGGAGGAATGGGCTTTTGNAAAAAAAACCCCAAAAGGAAACTTCCCGNGNCCATTGTTTCCAAGG
GCCAAAAAAAGGGNGGANGCTTTAANAAAAGNCCCCTTTATTTGGCCACANTTGGCCCGGGAAG
GGAGCTNCACCGCGGTGGCGGCCGCTCTAGAACTAGTGGATCCCCCGGGCTGCAGGAATTCGGCACG
AGGGNCCTCANCCTNTTTTCCCGGCGCCCGATGCTCTGTAAGAGCCTGCTCCCCATNGTGGTCNAGCAT
GTGACGAGCCACNCGCGGNCCCGCCATNAGGTCCAGGCCTGCCTGCTGGTCCAGAAGACGCTGCCCAC
AGGGGAGCTACGCGTGTGCTTTAAGGACCCAGAGNGGANGCAGCCTGATGGGCCAGACCCTGGGGAA
NGTCACCGATATCCTGCTGACGCTGCCAGAGGCTGAGACCAATGGCAGGGNACCANAAGGATNTGGN
GTCCTTGGAGCTGCTCANCACTCTGTTCAGGAATTTCCATCATGATAAGNTGACCATGGACCTGACCGC
CGTGGTGGGCCGTGCTGCANAGCCAGCAGCTAANGCTGCAGNANAGGCNACAGCAGGGGGAGCACTC
GGGCGGATCCNGCCGNCTTCACGACCTATACTGGCNGGCCATGAAGTTTCTCGGAGTCCAGCGCCCCA
AGTCAGAGAANGATACCAAGGAGGCCCCNTCAGGCCACACAGNTAGAGCCCCAGTTAGCATGANNGG
GAAGAAAAAGGGCTTCTTGCCAGAGACCANTAANCGCAAGAANCATNAANCCNTGNAGCCACCACTC
CATNGAGGCGGCGGGCGGCTNAANCCCGAAGCCACCAGTGGGGGACCATCCCCCCNNCGCCGGCCTN
GAAGAATAANAANTAANNAAGNNNATGAATTAAGAACCATNTTCCCCNCCCNCTTGTNCCGAAATGA
ANTGGGGACANCCTGGTTGCCAANNANTCNAACCCATAANGCCTCCTTGCTGGNNAGCCCCCNNNATC
CCCTANCCANATACCCCNAATNCCCCCAGAAAAAANACCCAAAANTCTGGCCCANNNANAACCNAAG
NCNNTCCCCTTGGCCTNANTAAATNCCGCCGGGGGNAGCCANTGNTCCTAAAAACNTNNCAAAAAAC
CTACCCCNAAC
GCNCTACTGGAGCTNCATCNCNGTGGCGGCCGCTCTAGAACTAGTGGATCCCCCGGGCTGCAAGGAAT
TCCGGCACGAGGCTCGTGTTCCGTGTCCTCCCGCGCTTCTTTGNTCGCTCTTCCGCACCCCGNGGCCCG
TCTCCTCCATCGATCGCCATGGAAGAAGAAATTGCCGCCCTCGTCATCNACAATGGTTTCTGGCATGTGC
AAAGCTGGCTTTGCTGGGGATGACNCCCCCCGGGCCGTGTTCCCGTCCATCGTTGGGCGTCCCCGACA
CCAGGGCGNNATGGNGGGCATGGGGCANAAGGACAGNTACNTGGGCNACGAGGCCCATAGCAAGCG
TGGCATTGTGACCCTTAAGTACCCNATCGAGCACGGNNTTGTCNCCAACTGGGACGACATGGANAANA
TCTGGCACCACACCTTTTACAACGAGCTGCGTGTGGCCCCCGAGGAGCACCCCGTGCTGNTGACGGNG
GCCCCTNTGAACCCCAAGGCCCAACCGTGAGAAAGATGACCCANATCATGTTCGAGACGTTCAACACT
TCTTGCCATTGTATGTGGCCATCCAGGCTTGTGCTTTNTTTTGTACNCCTCTGGCCNGCACCATTGNNNT
TTTCATGNCCTCTGGGGACGGGGTNCNCCCACACGGGGGCCCANTCTTACGNGGGGGNTCGCCCCTCN
CCCCCACANCCATTNCTTGNGGTTTGNAACCTGNCTNGCCCGNGAGCCTGAAAAANTTNNNNTNNTGA
AANATCCTCNCGGGANCGTTNTTTACNGCTTTAACNCCCCAACCCGGNCGGGNAAAATTNNTCCTCNN
NAATNNAAAGANAANTNTTNNGTTCCTNNAGCCTTGGNAATNNNNNACAAGNAAATGGGCTTTNCGC
TNGNNTTTTTTCTTCTNNCTTTGGAAAAAAANCTTTTTANCCTTTCNNAAGNTGNTNNGGTATNCNCCN
NTTGGCNANNGNTNGGGTTTCNGGTGNNCNNNNNTNTTTNNTCACCCACTTTTTNANGGGTATTGN
CNTCCTACCC
NAAAAGCTGGAGCTCCACCGCGGTGGCGGCCGCTCTAGAACTAGTGGATCCCCCGGGCTGCAGGAATT
CGGCACGAGGGTGTATTTTTAATATTTTATGAATGCCTTTTGTTCTCANTTTTGAGGCATAAACTGAAAT
TCTGAACGTACTGGAAAAGACTTTACTTACTTCTTGATACACTTACAACAAGCCTGTGAGAAAGTAGC
ATTATCTTTTTACTTATCCAAAGTGGAAAGGAATGGACTTTGAACTCAAAGCTAGGTCTAGTTAACGAC
GAGGCCTGTGCTCCTTAACTGATACATCAGACCACCTGGTTGCATGCTGCTTCTGGGCAAGCGAACTAG
TGCTGGATTGGCTAAATGTTCGTTTTTTGTTGTTCAATATGAAGTCCTTTTGGTCTTCAGATAACTAGAT
ACAGGATAAACAGTATCTAAGGGTTGTTGTTTTATTGTTTTAAAGCATGGATCAATTACAGCTCTTTTG
GGTTACACTGGAAATTTCTAGCCAGAAATTTCATGACTAANAAAAAAAAAAAAAANAAAAAAANNNN
CCCNGGGGGGGGGNCCGGGNCCCAANTTCCCCNTNANNGNGGGGGGTTNAANNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNTNNNNNNNNNNNNNN
NNNNNNNNNNNNNNNNTNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNTNNNNNN
NNNNNNNNNNNNNNNNNNNNNNNNNNNNCNNNNNNNNNNNNNC
GGNGNGGGGGNAGGANTCCACTNTTGNCTTNNNTTNTNTATCANTCCNNNNCTTCCCNTTCATACNCN
TCTCTNTTNGACNTGANTNNNCTCTNNCNACTCCAGNNANGGTNGNNGGTGANTNNANCNNTANNTA
ANNTAGAGANNNANCTGGGGNACTCNANCACNGNGGCNGACNCTCTANAAACNNNCTGGAGCCACTC
TTCCCNGCTCTCATNCCNTNNNCAANNAGNTANNTTATTTNNTTANTCCTAACCATTCNTCTCTNNNNN
NTGGGGATNNCTATTANAGNGNGTNACTCCNTCATNNTANCNNTCNTANANCTCGGGGNNCCACNNT
NTANTTTAGGCNNTNCNGATANTCNNNATNGNNATNCTCCATTNCNANTTNCNCGNCTNGACTATATT
TTATNGANNTNATNNATAAAAGANNNANNTNGCGNTCTTTCNATANNNNNGNTANNTNNNTGNTGNN
NATAGANNATATNGAANNATNTTGTTNCGNATTTNNNACTTACTNNTTGTANNNNANGTNTNNGNNCN
ATGTCNCCGANGTNTNNCTNNANNANGCANTGNNAGCNCGNNNGNTNNAGTCNCNTTNTGACNANGA
NTCACGNGNNNNNTACGTGCTNNTTNNNNTNGNTATTNNNNGNGAGGNNANNATANNGNCNNNNGTG
ATGNGTCTTTTGAGNATATTAGTGNNCATNTNNGAGGGGNGNAAGGTCCNTGNNTTNTNNTNTGNTCN
GNNTNANNNATGATGNCNNGGTTTNNNTATNNATNANCGATNCCNTGNGANNNNTCGNGTANTACTN
NTNGGGNTATTTGTNTCGTANNTGNCGATNGGNCNAGTTANGGCTTCTATAGTGTCTNGTANTCNATA
TCTTGGNGGNCCCCNCTTTATTANANGNCTNNAANNNGNAGNNNTNANNNTNTCACACNCCTANGAN
NNCACCCATNTCCTTNNTATNNTANTANNCATNATCTAGTTNNNTAATCATANCNNCTATTTCTGGNTN
AGATTGAGATGGTAACNCCTTNNNNNCTCCNCCTTNATCNNCGANGNNATNNNTCTANNAGANNCNT
ACGCNNTNCCTNCNCATNNANTTATTNNNANTACNTNNCNCCGATNTAACACGACNTGNNGGANNNC
GCNNANCGTAGGTAANAGTCNTANGNAGTAATTTNATNATGACTANNTGNACGGAAACTNNNTTACA
NTNCGTGAAACTATGGANGTTNNTATNANTGATNGNATTCGNAGGGGNCCTGTAGCGNTNNNAANAG
NNGNNGNTTNNTANGNTGCNNGTAACGTNCNATNNCNCAGCGANNNCGCAGTTNNNCNGTTGAAGNT
NTNTATGTATGTACCACGNCCGTAACNANGTNANATNNANGNACGNGCGTNTCNANCTAAATANACT
NGNACGCANGTACNCTAAGAGTCGACNTNNTCATCCTTGCTTACTCTTANNTCTCGNTTCTNCNCTNTC
TNCGTAAATCAATNTNACATAGANGTAANTCNNNCACAGNTAATAGNANCTACGGANATNTCCACNN
TCGTNTCTCNCCATTAAANNGAGTGAATGANTGAGNNTNTANTAACNTNTNACGNNAGNGTACNNNTC
NGNAGNCTNTTCGCATNNTGTNTGGTGTNNNNNACACCNAATGAAATANACAANTCAGCCGG
GGNGNGGGGGNAGGANTCCACTNTTGNCTTNNNTNTNTATCANTCCNNNNCTTCCCNTTCATACNCN
TCTCTNTTNGACNTGANTNNNCTCTNNCNACTCCACNNANGGTNGNNGGTGANTNNANCNNTANNTA
ANNTAGAGANNNANCTGGGGNACTCNANCACNGNGGCNGACNCTCTANAAACNNNCTGGAGCCACTC
TTCCCNGCTCTCATNCCNTNNNCAANNAGNTANNTTATTTNNTTANTCCTAACCATTCNTCTCTNNNNN
NTGGGGATNNCTATTANAGNGNGTNACTCCNTCATNNTANCNNTCNTANANCTCGGGGNNCCACNNT
NTANTTTAGGCNNTNCNGATANTCNNNATNGNNATNCTCCATTNCNANTTNCNCGNCTNGACTATATT
TTATNGANNTNATNNATAAAAGANNNANNTNGCGNTCTTTCNATANNNNNGNTANNTNNNTGNTGNN
NATAGANNATATNGAANNATNTTGTTNCGNATTTNNNACTTACTNNTTGTANNNNANGTNTNNGNNCN
ATGTCNCCGANGTNTNNCTNNANNANGCANTGNNAGCNCGNNNGNTNNAGTCNCNTTNTGACNANGA
NTCACGNGNNNNNTACGTGCTNNTTNNNNTNGNTATTNNNNGNGAGGNNANNATANNGNCNNNNGTG
ATGNGTCTTTTGAGNATATTAGTGNNCATNTNNGAGCCGNGNAAGGTCCNTGNNTNTNNTNTGNTCN
GNNTNANNNATGATGNCNNGGTTTNNNTATNNATNANCGATNCCNTGNGANNNNTCGNGTANTAGTN
NTNGGGNTATTTGTNTCGTANNTGNCGATNGGNCNAGTTANGGCTTCTATAGTGTCTNGTANTCNATA
TCTTGGNGGNCCCCNCTTTATTANANGNCTNNAANNNGNAGNNNTNANNNTNTCACACNCCTANGAN
NNCACCCATNTCCTTNNTATNNTANTANNCATNATCTAGTTNNNTAATCATANCNNCTATTTCTGGNTN
AGATTGAGATGGTAACNCGTTNNNNNGTCCNCCTTNATCNNCGANGNNATNNNTCTANNAGANNCNT
ACGCNNTNCCTNCNCATNNANTTTATTNNNANTACNTNNCNCCGATNTAACACGACNTGNNGGANNNC
GCNNANCGTAGGTAANAGTCNTANGNAGTAATTTNATNATGACTANNTGNAGGGAAACTNNNTTACA
NTNCGTGAAACTATGGANGTTNNTATNANTGATNGNATTCGNAGGGGNCCTGTAGCGNTNNNAANAG
NNGNNGNTTNNTANGNTGCNNGTAACGTNCNATNNCNGAGCGANNNCGCAGTTNNNCNGTTGAAGNT
NTNTATGTATGTACCACGNCCGTAACNANGTNANATNNANGNACGNGCGTNTCNANCTAAATANAGT
NGNACGCANGTACNCTAAGAGTCGACNTNNTCATCCTTGCTTACTCTTANNTCTCGNTTCTNCNCTNTC
TNCGTAAATCAATNTNACATAGANGTAANTCNNNCACAGNTAATAGNANCTACGGANATNTCCACNN
TCGTNTCTCNCCATTAAANNGAGTGAATGANTGACNNTNTANTAACNTNTNACGNNAGNGTACNNNTG
NGNAGNCTNTTCGCATNNTGTNTGGTGTNNNNNACACCNAATGAAATANACAANTCACCCGG
NNAAAGCTGGAGCTCCACCGCGGTGGCGGCCGCTCTAGAACTAGTGGATCCCCCGGGCTGCAGGCGG
GTTTGCCGCCGGGACACAGGTGTCGTGAAAACCACCGTTAAACCTAAGCCAAAATGGGAAAGGAGAA
GACCCACATCAACATCGTTGTCATTGGGCACGTAGATTCAGGGAAGTCTACGACGAGTGGCGATCTGA
TCTATAAATGTGGCGGGATCGAGAAGAGAACAATTGAAAAGTTCGAGAAGGAGGGTGCCGAGATGGG
AAAGGGCTCCTTCAAATATGCCTGGGTCTTGGACAAACTTAAAGCTGAACGTGAGCGTGGTATCACCA
TTGATATNTCCNTGTGGAAATTTGANACCAGCANGTNNTATGTAACCTTNNTNGNTGCCCNAGGNCAC
ANANNTTTCNTCAAAAACTNGATANCNGGCCCNTCCAAGNNNGNNNGNNTTGCCTNGATCGTGNTNG
NTGGGGTGGGNAATTTGAAGCCGGTNTCTCCAANAACGGGCANACCCGTGAGCATGCCCTTTTGGCTT
ACCCCTTGGGNGNGAAACAACTAATTGTTGGCGTTAACAAAATGGATTCCACTGANCCNCCCTATANC
CANAANAAATNCAAANAANTTGTTANGGAAGTCANCCCCTTTTTTAAAAAAATTGGGTTACAACCCCC
GACACAGTAACANTTTGGGCCAATTTTTGGCTGGAANGGGGAAAACCTTGCTTAAAACCAAGGGTTAA
ATTTGCCCTNGGTTTNAAGGGANTGGAAAAGNCCCCCGTAAGGGCGGGNAATTGCCCCNGGGGAAACC
NNCCCTTGTTTGNAAACCTTTGGGAATTGGNNTTTTGGGCCNCCAAATTTTNCCCNAAATTGGAAAAA
AACCCTTTGGGGTTTTGGCCTTTTCCCNGGGGNNGTTTTTTNAAAAAANGGGGGGGGATTNGGGACCCG
GGCCCCTTTNGGGGGCNGGGG
TNNNNCAAANCTGGAGCTCCACCGCGGTGGCGGCCGCTCTAGAACTAGTGGATCGCCGGGCTGCAGG
GTTCGATCCATGCCCATCCGAAAGGATGATGAAGTTTCAGGTTGTACGAGGGCACTACAAAGGGCAGC
AAATTGGCAAAGTAGTCCAGGTTTACAGGAAGAAATACGTCATCTACATTGAACGAGTGCAGCGGGA
GAAGGCTAATGGCAGAACTGTCCACGTGGGCATTCACCCCAGCAAGGTGGTTATCACCAGACTAAAAC
TGGACAAAGACCGCAAAAAGATCCTCGAACGTAAAGCCAAATCTCGCCAAGTAGGAAAGGAAAAGG
GCAAATATAAGGAAGAAACAATTGAGAAGATGCAAGAATAAAGTCATCTTGTCTACAGCTTTCATTAA
AAACTGTTAAAATGAAAAAAAAAAAAAAAANNTGGGGGGGGGNCCNGGNCCCNANTTCNCCNTATA
GGGAGTCGNNTNACAANNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNN
TNNNNNAAAAGCTGGAGCTCCACCGCGGTGGCGGCCGCTCTAGAACTAGTGGATCCCCCGGGCTGCA
GGAATTCGGCACGAGGCGCAGTATTTGTCTTCCTGTGACTGACTTATTTCATTCATCATGTCTTCAGTG
TTGTTGCAAAGAGTAGGATTTCCTTTTTAAAGGCAGAATAATATTGCATTGTGTATACATTCCATATTC
ACTCATCTGCTAGTGCACATTTGGGTTGTTTCCATTTCTTGGCTGTGGTGAGTGTGGTGTGGGGAGCCG
GCGGGAGGCACTCCACCCGTGGCAAAGGTCATGAGGAAGGAGGCTCGACATATGCAAAGGCGGGATC
GAGCCTCAGGAGTCCCCCTGGAAATTTTCGAGCATCTACCCCCATAACCAGAGCCTGCCTATTTCACTA
CTTTGTGCTCTCACCTACACCTCTGACTTTACGGGGGGCTGTCCCCCACCACCTCTTTCAGAGAAGGAG
TTAACTTAGAGCTCCAGTTAATAATAATTCCTGGGCATGACAGGAGTGTTCCAAACTTACCAACTCCTC
TGAAGGTTCTCTAGCCTGCCTGACAGGCTTGTCCGGCCCCATGTGATTGCTCACAGCCTCCGAACCATG
AAANGCAGGAGATGCGTTAAACCTTNTAAAAACAGGTTCCNTTAAAAAAGTTAANAAAACCCTTTAGT
ATAAGTATGGGGGGGCTGATTAAAAAATTGGATTGGGGGAAGGGTTTTTANTTTGTTGGGNCCAAGGG
TTAACTGGGTAAATTTTCCAAATNCCCTGGCCNTTTCCCCCCTTTTNTGGAATTTTTTTNNAAAAAAAT
NGGTTTTTACCCNTTTGATTTNAAAAACCGGTTNAACCCTTTNGGCCCAANAAAATTTTTTTTNTTTAA
TNAAAANCCCNTTTANCCCTTTNCCCCTTTAANGAAAAGGGACNTTTTTTTTGGGGGGGGGGGTTTTTT
TNAAAA
TTNANCAANACTGGAGCTCCACCGCGGTGGCGGCCGCTCTAGAACTAGTGGATCCCCCGGGCTGCAGG
GTTTTTTTTTTTTTTTTTNACCNGNNTNGGGGGTTTAANGGTTCNANNATNNNNNATNGTAANNAAACC
CCCCNNAGGGGGGCCCCNAAAAANTTNNTTTTNNTTTNAAAACCCTTTTTNGGGNNNANGGGGGGCCC
CCCCCTAATAANNCTNGGGTTGGNNNACCCNNNTTNGNNGAANGGGGNAAAAANNGCCCNGNANGG
NCCCNCCAANCNAAANNGATTCNTNCCNTTNGGGGGNANNAAAANTTTNNATTTTANNGGNCCGGGG
GCCCNGGNNNGNAANCNCNTTTNGNTTTTNGNNGGAANNNGCNGGNNNNNNGGGGGCCCCCCNGNA
NNGNNNNTNGNGGGNGNAAAAANCNTNGGGGNNGNNANNGCNCCNTTANNNAGGNANTGGNGGGTN
GTTTAANAAATNCCANNNGNGNGGTTCCNAANAANCNNGGNNNGAAAAAGGNTTTTNGGCCANGGAA
ANCCNTNTTNGCCAAGGGGNANANCCTCCCCNTTGGNGANCTGGAACTTTTTTNCNAGGAANAAAAA
GNAGGTTCCCGGGCCCTTTTTNTTTAAAATNCNNGGCCCNCNAAACNNAATTTTTTCNTTGTTGCCCC
CCCAAAATTTNCCCNNNGGGGNGGGGGCCANAAAAGGAAANANCCCTTTTTTTTGGGGAGAAANTTT
TTTGAGGNAANTTTGNNAAGGGCCCGGCCCCCCCCNNGGGNAAANGGGCCCNNTAATGGGNTTTGNG
GGGCAATTNCCTTTANCCCCCTTTNAAAAAAAAAAAAACATTTGGGGGGGGGNAACNCCNCTNCCCCT
TNAANTNCCANNAAAAAAANACCCCCCTTTTGGGGNGCGCCCAAAACCCTTNTGGGGGGGGGAGACC
CCT
CANNANCTGGAGCTCCACCGCGGTGGCGGCCGCTCTAGAACTAGTGGATCCCCCGGGCTGCAGGTTTT
CATGCAGCAGGCCTTTCCTGTTGTCATTGATGTCCTTACCCCCCTTCTCTCCCTETGGTGCCTAGTTAAC
CTTTTCAAATGCAGACGTAATGGCATTGCTTTTTTCTGCCATAGCTTCAGTGGTCCCCGTTATGGAGAG
TAGAAGTCATAAAACTCAAATCCCTACAGGGACTAGAAAGTTAAGGTAAATGGACAAAGTGAGATGG
ATGTGAGAGGAATGGGAGGAGAGAGGAAGTGCTGTGGCATATTGTCCCGTCTGAAAGGGGCACCTGC
CACCTGCTTCCACGTGGGAATGCGGGACCCAATTTGACATATCGTCCAATTTCTTAAAAGAAGCCGAA
AATCCAGAAATTTTTATGAGCGGTTTGCTGATTTTTAAATATTGACTTTTAGTTGAGTTTTTAAAATGCA
GGTCAGTATTTTGTGAATTGACCTACTTGCAGTTTTTGGAATGTGCCATTTTATCTTGTGATTCTGTTTG
TAAGTGATGCTTCTTCTAAATAACGCTGTTTGCTTCCTCCTGCCTCAAAACCTGGCAAAATTAGGTTCTT
TAAGATCCAGGTTAGCCTTTCCTGACCCCAAGACAGGCAGTTCTCCTCGTGTTCTCCAAGCTCTAGCA
CTTACTACATTACATTGCAATGCTTTGNGTTTTATTCCCTGGCTTTGAGCAAAGACATTATTTTATTCTT
CTTGCAAAATTATACACTTTGGTAAGTTAAATATTTTTTGCCATATAATGGATTAAACTCTTTANTTTTC
ATCTAATGGAGTCAAGNATCATAATNGCTCTTCATTGAATTTCNCTTTCTCCCACCATATTTTTAAAACC
NAAAAAATATTTTTCCATAANGGAANNCNTANTTTAAAGNAAACCCCCCTTTTGANACTTNAATTTGG
CATTGGCAAGGTTAAAAAANCCG
AAANCTGGAGCTCCACCGCGGTGGCGGCCGCTCTAGAACTAGTGGATCCCCCGGGCTGCAGGAATTCG
GCACGAGGGAGATCTCTGCGAACAGTTCAACTCCATGGAGCCCAACAAACAAAAGAATGTGTCTGAA
GAACTGGACAGAACCCCACCTGAGGTGTCGAANAAGCTTGAGGACATCCGGACTCGTTATGCCTTCTG
AGCTCTTTTCCTGTGGGGTTTATCANANACTTTATTTCCCTCACCACGTGACTGTGAATCTCTGTGACA
NAANAAGGGNGGTGATAATTAATACTTTGTAATGTTAAAGGTCTTAGCTCTTCGCATCCACTCTTTCCT
GGAGTGACAGGTTCCCCCTAAAATTGGCACTGAGATTTGCTGCACTTCTCGGTGCCTGGTACATAATCC
ATTGCCCCCAGGTTNTGGTGTGGAATACAGTGTCCCTTTTCCCAAAGCTTTCTTGCATTGATATTTGGC
GGCTATCCNGCCTACTTTNGTACACACCTTTAATTNTNANCTGGACCACCTGTACATACAGTTNTGAAT
GATGCTGTCTGTGGGGGNNCTAACNNAGCNGCCGACCAGGTTNNTTATANCACAGNGTCTGGAGNCCT
GNCTGTATCCAACCNAGCNATGAANACNTNCCTTTTGTNGNGAGGNGANNNNNNNNNNNNNNNNNNN
NNNNNTTNNGGGGGGGGGGGGGGGGNCCCCNNNNCCCNTTTTTTTNNNNAGGGGGGGNNNNNTNNN
NNNNNNNNNNNNNNNNNNNNNNNNNNNNNNNNNNNNNNNNNNNNNNNNNCNNNNNNNNNNNNNN
NNNNNNNNNNNNNNNNNNNNNNNNNNNNNNNNNNNNNNNNNNNNCNNNNNTNNNNNNNNNNNNNN
NNNNNNNNNNNNNNNCNNNNNNNNNNNNNNNNNNNNNNNNNNNNNCNNNNNNNNNNNNNCNNNNN
NNNNNCNNNNNNCNNNNNNNNNNNNNNNNNNNNNNNNNCNNC
NNNNATGTGGAGCTCCACCGCGGTGGCGGCCGCTCTAGAACTAGTGGATCCCCCGGGCTGCAGGAATT
CGGCAGGAGGCTCCCATCTGTCCAGTTAACATTTCCCTCTCTGTTTTCGAGATAGCTCTCCAAGAGGCT
GGGGTTCCTCAAATCTCATTTAGAACCGTGTGCTGAAAACCTGGATTGGTGACTGCCATGCTANGCCG
ATGTGCTGGCCCAGGGGAAGGAAACTGAGCGAGTAGAAGTCCCTACCANAAAGGGTTGGGTTTGGAG
AGGGGCATCCAGGGTACTGAAGTCCTANGTGCCAGGTATCTCCCCGCTCCTCTCANCTTGNGCAGAGG
TGTTGACAGTATTATCTTCATTTTCAGTCTCCTTCCAAGTTCTGTCCTAAGCTATTACCTTTGCAAGTTT
CTGGCCCCCACCCTATCAAGTGGGAATCCCTTCCTCTTCCACTTTATCTCCTTCATAATAACGTTGAATT
CCCTCCTTTCTCCTGTTTGAGAAAGGGGGTCAAGAGAAGCAAGAGATAGCCTTGGTCTCTTAAGTCTCC
AGAAAGCCCCTCCCCACCCCATGTTTTCTTATCAAAAAAAAATGATACGTATCAAGCCCTTCTGTACTT
ATATGGAAAGGGTATGTGCTGCCACCTAAACGTCATTTCCCCAAAACAGGGGAAGAAGAACACCTTTN
TATCTTTTTGGCAAAACAATCTTNATNTCNTCCTGGCTACTGNNTTCCCCACTTTNTTTGGATTTTTGGC
CCTATTTCTACACTNTCAAATTTTCTATTGGGGGNTGGAACGTTGGCTNGCNTTTTTTTTTNNTAATTGG
AAAANAATGACCATTGGCCCCCCANNAGCCCCAAAAATTNNATGGGGGAACCNGNAAAAATNTTAAA
AAAAAAANAAAAAANACGTTCNNGGGGGGGGCCCCCGGGTNNGCCAATTTNCCCCCTTTTTNAAGNNN
GNGGNC
GGGGNNGNGNACNAAATNTTAATTNNATNTTNNNNTNNNNCCCNCNCNCTCNCNNANTATTNTTTATT
GTGGAGGNGGGNCNGACCTCTACNNNGGGGGNGNGNTGNTAGTTACCNAACATATTNTATCCATTAG
GAGCTCTATANTNNTTNTNGNCNCATTTCTTTNATANTGTNCANNNGAGGGNCNCNGTTANTNNNCCNN
NNNNCTNTTTTTNACTTATATNTTCCATAANTCNGANTNANTTTNAGNGGNTTNTTATNNCGNTTAGCT
CANAGTGCNCNTCTGNNAGNTCNTTNCNTTAAATGANGCCCNGTCANNTNTATTANAANATNTGNGTG
CNNNNNCAAANCAGNANGNTGANNTNGGNCGTNATTTTGATNNANTNNCTATGTGCGANTTACNNGA
NGCTGACNTNGAGNANNANNGGNCCANAGNNNATCNCGGCCNANNACACANTTGACAGTANTGNNN
TNNGNGTAAANNAGATGANNNCGCNTGAGNNTTGTNTTCNCNTNCGNAANNCNCNCNNTCGANNATA
CNCGTANTNGAGNTTGACTGNNNGNGAGTANGGANANGNCANNNNNNCGATGATNNNGNNNNNNNT
NNGCAGGTCCGAANNNCNANAATANNCNNGAGTNNNNNNTACNGAGNNNNNGNNATAGCNTNNNGTT
NCNNTCCNGTTANNNATNTTNNNCNCGNNANGNGNTANNCGCGNTGTANNNNANNNTGGAANNNNA
CGAGANACCGNNAGATATNNATNACCNTNNTATNNCNANANAGGTCCANCTANTNNCNTTCGNNGTNA
TCTNTCNTNNNCGNNNGNATCNCGAGGNANNNCCAACTNCACNNACGNNNNNNCGGTCNNCATNNNC
NTGNNCTATGTNCTACACNGNGTCTCCTCNCNTNTGTNNACCNTATAAGNTGTCCTACGNGTAANACT
GANNNAACGTCNNACGTCCNNTATANTCGGGANCANNCNATACGTAANGTNCTAGNAGCGNCANNGT
NGTGACTTCTNANTANCANATCNTGANTNNAGGTTANTACNTACNATNNNACNNCNGAGATTANTNN
CGTNNTNNCNNNNGNTGATTTNTAACGCANGACCNCCATCCACGCATGATCATANTACGNTGGANCN
ANTNCGNNNTANTNTGNANACNNTCTTGTTCGTNNGNNNNTCNTCATCANGNNCANANNTNCTACGTN
GANTNGTCCACCTTAGCGANGCNNTTNTAANTNTANNNCAGATCCGNCCTCACCACNGTANTGNCTNG
NTNANNNTNATCGNCGCTCCCGCANNACTANCTACNNTAGGAATANNCACNTNNTNNNTNATNGNTC
GCACGTAGNANANACNTNNNCANNNTAGNGCGGTAGTNAGNGGTCCGCNCTGTACGTNTATATCNCC
NACNNNATGTCTGTGCGCANCACGGNTTACGNCNACACGNTNGGNNGATGATAATNGATNGATCGCT
CGACCGNNGGTNATGNNNNNNTGCANNGAANNNCGNAGTCGTANGCTACANAANNTNTNNANNNAT
AATAAANAAANACAGGGNATGNNANTATNTATGATATGGGANGNGTNNTTCTGNTTGANATNGNTNT
NNACNGNTNTNTAATGGATGGANCACGANGATANGNNNTNNTNTCNTTATGGNTGTANNNTATNCCA
TGTNCGCGNACNCNATGNGTAANGATGCNCANGCAACGAAGTNACNTNNTNAGGNNGACNTGNATGC
GG
GGGGNNGNGNACNAAATNTTAATTNNATNTTNNNNTNNNNCCCNCNCNCTCNCNNANTATTNTTTATT
GTGGAGGNGGGNCNGACCTCTACNNNGGGGGNGNGNTGNTAGTTACCNAACATATTNTATCCATTAG
GAGCTCTATANTNNTTNTNGNCNCATTTCTTNATANTGTNCANNNGAGGGNCNCNGTTANTNNNCCNN
NNNNCTNTTTTTNACTTATATNTTCCATAANTCNGANTNANTTTNAGNGGNTTNTTATNNCGNTTAGCT
CANAGTGCNCNTCTGNNAGNTCNTTNCNTTAAATGANGCCCNGTCANNTNTATTANAANATNTGNGTG
CNNNNNCAAANCAGNANGNTGANNTNGGNCGTNATTTTGATNNANTNNCTATGTGCGANTTACNNGA
NGCTGACNTNGAGNANNANNGGNCCANAGNNNATCNCGGCCNANNACACANTTGACAGTANTGNNN
TNNGNGTAAANNAGATGANNNCGCNTGAGNNTTGTNTTCNCNTNCGNAANNCNCNCNNTCGANNATA
CNCGTANTNGAGNTTGAGTGNNNGNGAGTANGGANANGNCANNNNNNCGATGATNNNGNNNNNNNT
NNGCAGGTGCGAANNNCNANAATANNCNNGAGTNNNNNTACNGAGNNNNNGNNATAGCNTNNNGTT
NCNNTCCNGTTANNNATNTTNNNGNCGNNANGNGNTANNCGCGNTGTANNNNANNNTGGAANNNNA
CGAGANACCGNNAGATATNNATNACGNTNNATNNCNANANAGGTCCANCTANTNNCNTTCGNNGTNA
TCTNTCNTNNNCGNNNGNATCNCGAGGNANNNCCAACTNCACNNACGNNNNNNCGGTGNNCATNNNC
NTGNNCTATGTNCTACACNGNGTGTCCTCNCNTNTGTNNACCNTATAAGNTGTCCTACGNGTAANACT
GANNNAACGTCNNACGTCCNNTATANTCCGGANCANNCNATACGTAANGTNCTAGNAGCGNCANNGT
NGTGACTTCTNANTANCANATCNTGANTNNAGGTTANTACNTACNATNNNACNNCNGAGATTANTNN
CGTNNTNNCNNNNGNTGATTTNTAACGCANGACCNCCATCCACGCATGATCATANTACGNTGGANCN
ANTNCGNNNTANTNTGNANACNNTCTTGTTCGTNNGNNNNTCNTCATCANGNNCANANNTNCTACGTN
GANTNGTCCACCTTAGCGANGCNNTTNTAANTNTANNNCAGATCCGNCCTGACCACNGTANTGNGTNG
NTNANNNTNATCGNCGCTCCGGCANNACTANCTACNNTAGGAATANNCACNTNNTNNNTNATNGNTC
GCACGTAGNANANACNTNNNCANNNTAGNGCGGTAGTNAGNGGTCCGCNCTGTACGTNTATATCNCC
NACNNNATGTCTGTGCGCANCACGGNTTACGNCNACACGNTNGGNNGATGATAATNGATNGATCGCT
CGACCGNNGGTNATGNNNNNNTGCANNGAANNNCGNAGTCGTANGCTACANAANNTNTNNANNNAT
AATAAANAAANACAGGGNATGNNANTATNTATGATATGGGANGNGTNNTTCTGNTTGANATNGNTNT
NNACNGNTNTNTAATGGATGGANCACGANGATANGNNNTNNTNTCNTTATGGNTGTANNNTATNCCA
TGTNCGCGNACNCNATGNGTAANGATGCNCANGCAACGAAGTNACNTNNTNAGGNNGACNTGNATGC
GG
TTNANAAAGCTGGAGCTCCACCGCGGTGGCGGCCGCTCTAGAACTAGTGGATCCCCCGGGCTGCAGGA
ATTCGGCACGAGGCNANCNNNNAGANNNNANANNTGACCNNGGATCANACCNATNANTTGAAGGNN
GNNCTTCGAGCTGNGNTACCGACCGGGGGANGNATNGATCCTGTACAGATANNGTGGNCATGAGATT
AGGGCCCTGTTCCAANCCTNNCCAAATGTTACGGTNNNCANTGNCTTGCGNAACCCCCCCATNNACGA
ANNNNACGANNAGGTTACTNANCNTTGGGNNCTTGCTNCNCANGNTNNTGACTNNGNGNCATAANAT
GGANCTNGGCCNNTACAATNNNTGNNCCANNCGTCTNAGCGTGTNCNTNAACNANANGAACGGNCNT
TNTACTGGGTTGCTTATTGGCCGGGNCCTNGTNCNTTACANTGGGATGCNATCNTGACAGNGGATCAA
TTANATATTCCCNTAGCCTTGGAATNAGGACCTTAATTTGCCTTGNNCAANTNAAAACAAGCCNCCCCC
NTANGGGNGGGNAACCGGCTTATGGATTCCGGCCCCATNAACCGGAGGGNGGCCCTTTCTTNTNGGA
ATTTTTCCGGGGCTTTNGGCCGTTTTTCNTTTTAAGGGCCTTTTTTTTTCNAAAANNGGGCCCNCCCANT
TCCCTTTTTAAANNAGAAAAATNTNCGGGGGACCNAAANNGGTAAAAAAAAANCCCTTGGGTTCCCC
TGTTTNTTGGGGCGGNCNNAAACANTTTGGGTNNTTTTTTTTNTTTTTNGGGGGGGANGCTTTGGNACT
TCACCTTTTGGCCCGGCNAAAAGGCCCTGACCCCGGGAACCNTTTNTTTGGAANCTNGGACNTTAAAT
GGGTTTTTTGGGCCCCCCCCCNTTATGGGTTANNCCTNGGGNCCNTTTAAANGNNANGGGGGCCCCNG
GGCCCTAAANTTTTTTTTTTTTTTTNCCCCCCCCTTTTNAAAAAAANTTTNCCCCCCTTAAAAATTT
TTGANAAAACTGGACTGCCCGCGGTGGCGGCCGCTCTAGAACTAGTGGATCCCCGGGCTGCAGGGATA
GNNTNATGCCNATNTGANGGNAATATTAAACAANGCACGCCCNAGGGCACTANAAAGGNCATCATAT
TGTCAANTTAATNCAGGTTTACAGGACCAANTACGNCATCTCATTGAACCGAGTCAGCCTGANAANNN
TATTGTCACNCCTNTCCNGNCGTACATNNANNCCATNNCNNCGNNNATNACNATACTTATNATTGNCG
NTNATANCCGANNTANCATCGTNGTNTAGCNAGNNNCTCGTCNTTTATNAAAGNATTCAGGGCAAAT
NTNAGGAAGACTCACTTGAGAAGATNCNATAATAAAGTTATCTTGTNTNCATNTTTTCATTANNAACT
NTTANAATGATAANNATTTAANTTTNTGGGGGGGGNTCCCTTTTTCCTTTTTTCCNNTTAACTNNNCCG
TTTAACNCNNNCTACTTCCTTTNTNNTTTTTGTCNTTTNGAGANTTTATTTTTTAATTTGTATTTTTNNNC
TNTAACATTTANCCTCTNTACNTTTTTGNTTTTNTNNNTTTNTTTTTNTATTTNTNNNTNTTNNNTTTTNT
TTNTNNTTTNNNTCNNNTTNTNNTTCACATTANTCTTNTTCTTCTNNTTGTNNCTNNNANCAATTNTNTT
TTTNNTCNCNNNNTNNCNTCGCTTATNTTTTTTANTNTTNTTNNNTGNNTTCNTTNNATANANTTTTNNT
TTTCNTTTCTNCTTTTNTAACNTNTACTAATANTTATANTTTTCNTTANNAATTTTCTCTCNTCTTCCTCT
TGTTTTTNTTNNCTATACACNTNTATATTTTTATTATTTNTTTCTTCTCNTTANCTCTATNCTCTTTTATTT
TTTTTCNTTNATCCTTTTCATNCTTCTTTTACTNTTTCTAATTTCGTATTTATNTCTTGTGANTNTNNACG
NTTAATNTTTTCTTCGNGCNTCCTTATCACTTNTTCTGTANNTNCANCTTCCTCTTCCT
NNCAAAAGCTGGAGCTCCACCGCGGTGGCGGCCGCTCTAGAACTAGTGGATCCCCCGGGCTGCAGGA
ATTCGNTNNNANGCCTACAAACCNTCCAACCANNCGCACTGNTACGGNAAAAGCNGCTTNATCGTCAT
ACGCCTTTNTTACCAGCATAATGNCCACATTNATATTTATCCACTTCGGGNCANGAACTAATTANTTCA
NACTGGNCACTGGACTAAACCATCCCAANTCTTAAATTAATCCCCTTAAGCNTTAAAAAAGANCTATT
TTCTTCATNAATATTTAATCCCAGTAGCACTAATCCGGTNCATGGATCTAATATAGAAATGTTAATTTG
NATATATATACCTCAGANCCCCATATTAANCAATTCTTTCAAATATCTACTTCCTAATCCTTAATTCTAT
GCCTAATCCCTGGAACCCGGAAANCACCCTTTTTCAGGCTTTCATTGGCTGGAAAAGNGGTCGGAAAT
CTTATATTTTCTCCTATCGGAAGGAGGATNCGGGNCGAAGNGAAGCCAACCCCAGGCAGCCCTTCCAG
GCAATCTTATATTACCGNATTNGGCGACTTGGGTTTATTTTAAGCAATAAGCANGGTTCCTAACCAAAT
CTTCATTACCTGGANACCTTCCACAGGATCTTTATTCTTAAACCCAACGAACTTCAACCTTACCCTTGG
ATTGGACTAACANTTAGCTGCCANCCGGAAAATCCCGCCCAATTTGGGCCTCCACCCGNGAACTTTCC
CTTTTGGCAATAANAAGGGCCCAACTTTCCCGNTTTAAGANNTACTNCATTTCAAGCCCCAAATAANG
GGGAGCCAGGGATTNTTTCCCCACCTAAAACCGGGTTNTTTTCCCCCTCACCGGNAAACCCATTTAANT
TCCCTTCCCAACCTNTTTACCNTTTTGGCTTTNGGGNGCCCNTTTCCCCCCCTTTTTTTTCCCGGGANNT
TTGGNCCCCNCNCGCCCCCAATGGGGCCTTTTAAAAAAAAAATTTGGGNTTTTTCCCAAAAANANANA
AAAAA
TTNANCAANNCTGGAGCTCCACCGCGGTGGCGGCCGCTCTAGAACTAGTGGATCCCCCGGGCTGCAGG
AATTCGGCACGAGGCACCAGCCCAGGCTTGGACAATGGGNGGGGACATGTTCAACGCCAGGTTCATTC
GAAACTTGCATGAACGTCGCAGCACCAGGCCTTGGTGACCGCAGCGCCCTCTCACACCTTCAAAATCA
TTATTATTTCTATCTCACACAGGACTTAGTCAAAGCTTGCCCTCGCCGTGTATGTATTCATTCATTCATT
TATTCATTCTACAGGCTTTTTTTTTTAAAATCAGGGCCAAGNCATTTGNATCTTATTTTTAACCANAGCA
ATAAAGTNTTTTTTACCACGANAAAAAAAAAAAAAAAANNNNGGGGGGGGGNCCNGGACCCNATTTC
CCCTTNNNGGGNGNNGATNAACNNNCGGGGGGGGCCNGGNCCCATNNNCCCTATAANANGNNANNN
AAAANNNNNNNNNTNNTNNGNANNACNNNNNNNNNGCATNNTTTTNTTNTCTAANNAANGGGNNNN
TNATTNNNGNNGNNTATNNCNNTNNCNTTNTTNANNNNTTTTTNNNGNNGNNNNNNANNANTNN
NANNNNNCNGNNCCNNTNNNNNNNNANNNNNNNNNNNNNNNNNNNNNNNNNNNNNNNNNNNNNG
NNNNNNNNNTTNNNNNNNNNNNNNNNNNNCNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNN
AAAANCTGGAGCTCCACCGCGGTGGCGGCCGCTCTAGAACTAGTGGATCCCCCGGGCTGCAGGAATTC
GATATCAAGCTTATCGATACCGTCGACCTCGAGGGGGGGCCCGGTACCCAATTCGCCCTATAGTGAGT
CGTATTACANNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NTNNNNNTNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNTNNNNTNNNNNNNNNNNNNNNNNNNNNN
NNNNNTNNNNNNNTNNNNNNNNNNNNNNNNNNNNNNNNNNNNNNNNNNNNNNNNNNNNNNNNNNN
NNNNNNNNNNTNNNNNNNNNNNNNNNNNNNNNNNNNNNNNNNNTNNNNNNNNNNTNNNNNNNNNN
NNNNNNNNNNNNNNNNNNNNNNNNNTNNNNTNNNNNNTNNCNNNNNNNNNNNNNNNNTNNNNNNN
NNNNNNNNNNNNTNNNNNNNNNNCNNNNNNNNNNNNNNNNNNTNNNNNNNNNNNTTTNNNNNNNN
NNNNNNNNNNNNNNNNNNNNNNNNNNNNNNNNNNNNNNNNNNNNTNNNNNNNNTNNNNNNNNCNN
NNNNNNNNNNNNNNNNCNNNNNNNNNNNNNNNNNNNNNTNTNNNNNNNNNNNNNNNNNNNNNTNN
NNNNNTNNNNNTNNNTNNNNNTNNNNNNNTNNNNNNNNNNNNNNNNNNTNNNNNNCNNNNNNNNC
NNNNNNNNNNNNNCNNNNNNNNNNNTNNNNTNNNNNNNNNTNTNNNNNNNCCN
GCCTATAGNGAGTNGNATTACAGTGGCGGCCGCTCTAGAACTAGTGGATGCCCCGGGCTGCAGGAATT
TCGGTATCGAGGCNCNNCGNTANCNCAGGCCTCTCGGNTTNGCCGGTCCNCCTTNNCCGCCNATNGAG
ACCGNAANCANNTCGCCATGGATGATGATATTGCTGCGCTCGTGGTCGACAACGGCTCCGGCATGTGC
AAGGCCGGCTTCGCGGGCGACGATGCTCCCCGGGCCGTCTTCCCGTCCATCGTGGGGCGCCCCCGGCA
CCAGGGCGTAATGGTGGNCATGGGCCANAAGGACTCGNACGTGGGGGATGAGGCTCANAGCAAGAG
AGGCATCCTGACCCTCAAAGTACCCCATTTGAGCACNGGTATCGTCCACCAACTGNGACNACATGGAN
AATATCTGGCACCACACCTTNTACAACGAGCTCCGTGTGGCCCCTGANGAGCACCCCGTGCTGCTGAC
GGANGCCCCNCTTGAACCCCCAAAGGCCAACCGTNAGAAAGATGACCCCANATTNTNTTTCTANACCT
TTNAAACACCNCCTGNCATGGTACTTGGGTCATTCCAANGCTGTGCTGTCCCTTGNATGNNCTCTGGCC
CGCACGAACTCNNCATTTNNGATGNACNNCNGGTGACCNGGGTTNAACCACACNGNTGCTCCATCTTA
TNAGGGGGTNCTCNCTTTCCCCNTGCCNTTCCTNGNNTTTNGGAACNTNGGCTTGGCCGGGGAACNGN
CTGGANTTACCTTANTGNAANCACTCTTCNCGGGNNCGNGGNNTATTNNNCTTTNACCACCCACNGGT
NGATNNCGGNAAAATNNTTCCNTTNANNTTTANNTTAGAAAATTTTTNTCTNATNTGGGCCTTGNATTC
TNANCAANAAAAATGTCNCNNAGTNNGCGATTNANANTTTCTCCTTTGNNANAGNAAGNTCNAAACT
NTNNTNCTTGGNTCNNGAGTNTTTANNCTANAGANNTNNAGGCNNTCTCCANCCCCCCTGTNNNNTTTT
TCNCCAGTTTCCTTTTCTTNTGNAATGTAATANNTGANCC
NCAANATCTGGAGCTCCACCGCGGTGGCGGCCGCTCTAGAACTAGTGGATCCCCCGGGCTGCAGGGCT
GGGCATTCTGGTGTACACCATGCTGGCGGGATACACTCCATTTGCCAATGGACCCAGTGACACACCAG
AGGAAATCCTGACCCGGATCGGCAGTGGGAAGTTTACTCTCAGTGGGGGAAACTGGAACACAGTTTCA
GACACAGCCAAGGACCTGGTGTCCAAGATGCTGCACGTGGACCCCCACCAGCGCCTNACAGCCAAGC
AGGTCCTGGAGCACCCGTGGATCACCCANAAAGACAAGGTTCCGGAGAGCCAGCTGTGACATCAGGA
CCTGCAGCTTGNGAAGGGAGCCATGGCAGCTACCTACTNANCACTNAACANCTNCAAGCCCAGCCCTN
ANCTGAAGCCCATCGAGTCGTCTATNCTGGCCCANCGGCGGGTGAGGAANCTGCCGTCGACCAGCGTG
TGAGGGGCCTGAGGATTCAAGCCGCANGGCGGTGCTAGCTGGATGGAGGCAGCGTGCTTCCCANAAN
AAGCAGGCCTGAATCCCAGGGCCAGAGGGAGCGGAGCCCCGAGGGGCCCAAGAAAGCAGCCCAGCC
CAAAATCACCCCANGGAGGGNGNTGAGAANTGCCCTTCTCCTTNCCCCAGGAACNGACTGTTCTCGGC
TCANGCTGTGGGTGGGNTTGAAATCGAATTCACTGTACAAAACTCTTTTATCTATTTTTTTAANGACAA
AAATGGGCNTCTACCCNCCCATGGGATTTTTTTACAAAGAATNCATTTTTGGCCTTTTTTGGGGAAAAA
CAAANTTATCCCCNTNGGAAACCCCCCAAGGAAGGGGGGCTNGAATTTCAGTTCAAANGGGCGCCTCCT
TGTGGGCTNGAAAACCCCCTTTAAANACAAACTTTNANGAATTTNCCCCCCAGGGGGGAANCCCCCCC
AAATTGGGGTGTTTTTTATNCCCNNCC
GGNNCGTGNNNNGTNTTTTNTTTTTTTTTNTNTNTTNNTNNNNCCCTACNNACATACTTTTAATTNNAN
NTNTTAANGCTNTNNCTNGGNGNANTGANNTGNATTNAAACANTTTNAAACCANTGGACTNCACGGGC
TGGCGGCCGCTCTANACTAGTGGATCCCCCGGCCTTGCANGGNAATTCCNGCACGAGGCCCNACTCNG
CCCACCGGTGGCCCANAAGAAACTTGGNNTATTGANCNAATNAACCCTNTNTGANNAANTACGGCCC
NGGTATNGGGTACTNNCNAAGAAGCNTTGACCCNGNCTGGANTTTCCANTTTCNGAGGNATGGGCG
GGCANAGNGGNCNCGCNNCCCTGNCCGAATGGGGCCNNTCCNACCTGNCTTTTTNCTTGNGCCCNGNA
AGGNCCAACCNANAGGCNTTNCANAAANCTTTANANNTTNCAATTGGNNAGAAGNTTTCNCNGTNNC
TGCNTTGGGCCNTACTNAANNNNGTAACTCCNATANAAANGCTCCTTGTNTTTCTTTNATAGGCTCNN
GNACGNTAGANNTNCNTGAATNNCNTGNNCCCCCAGGGGNCANTNNTCCCTGTTTCTCCNTNACANNN
GGNNTNCNCCTNTCGAATGNGCTGGTNCNNNANTTATCACCNCGAAACAANCTGCGGCGGCNCCCGA
AGAACNNTTGTACCAGTTACATNTAAAATTAACTAAATANAAAACTTCTCCCNCCTTTNTCCCANANA
ANTNCNTANNTTTTNGTNCCCCCCNNNGNANANCACTCCNGNANNCNCCCNAGAATNCACNNCAANA
NTTTCCNGATTTTNTAACTAATGNNGGNNNCNACNNNCAGGTTNTNANCTCTTNNNANAAAAGTNNTN
NTTNTNNCCCNATNCCCAANTNGGNGTNTNNTCTTTCCTCCNNCTAATCTNCATATAATNNTCCCNTT
NTTTTTNTTNGNAAAATACACTCANGCNTTANATAACNTCCACCTACTTNNNTCTANTACCCCNCTNTC
NACTTCTNACTCACNCACANGNTNGGTNTNTGACGTGGATCTTCCNTCTCNCNNAGNACGTNCTTNTC
GTNNNAGAAANATNAATNNTCANCGNCNAACTACTTGNNCNNCGNTTCNTAGTANCTNNTCCTACGA
CTNNNGGNCNTAGTATTCNNTNNTATGTCGTATNTCCCACCTCNNNANTATGCNTNGCCNTTGAANCA
ACNCTNTCTCAATNTNGGNANGGNNTNGTTNCNNTNTCCNTTNTNNTCANGNAATNACTCTANGANAG
NNGTATTNCCNNTATCNNNTCTTCTCGNTACNGNNGTTCGGTACGNTNNNANNCTNCAGTCGNNANNA
TGTCGTACGTCTGATGCANNCNNNGCNCGGTANCATCTNCTTATNTNCNGTTCNCANGATCNANTANT
GATTNTCGTATANGNTCNCCNACTNNCANGNTNTCTCNTATGAATCNCTTTCTCTATNTATGCTNCGTN
GCNAAGANGTNNGCATGTGTNCG
GNCNCNTGNCNNGTTNATNNNTTTTTTTTNTNTNTTTNTNNNNCCCTACNANATACTTTTAATTTTNAN
NTCTTAANGCNTNNCTNGGNGCGNTTGNTATGGATTCAANCTNTTNAAACCANTGGACTNCACCGCGT
TGGCGGCCGGTCTANACTAGTGGATCCCCCGGCCTGCNNGGNAATACGTGNACGAGGCCCNNCTCAGC
GGANCGGTGGCCCANAAGAAACTTGGNNTATTCANCNAATAANCCCNTATGGNNNAATTAGNCCCCN
NNTNGGTACTNNCNAAGAANCNTTNACCCNGNNGCNNTTTCCANTNTTCNNAGGAATGGGCNGGCCN
AGAGGNNNCGCANCNCTTGNCCGAATGGGGCCNNTCCNACTGNCTTTTTNCTTNGCCCNNNAAGGNC
AACNANNGGCTTNCANAACATTNNANNTNCNATGGNAGAACCNTGCNNGNCNGCNNTNGGCCNTACN
AACNNGGNAACCNNAANAAANGCTCCTGTNTTGCTTTTATAGGCTNNATCNNTAATTTNCANAATNNC
NNGNCCCCAGGGGGNCNNTNNTCCCTGTTTCTCCNTAACANNNGNNTACNCCTNTCGAATGNGCTGNT
NCCGNANTTATGACCNCGAAACACGTNNNCGNCCCTCGAACANCNNTTGTANGCCNNTTACNTNTAAN
ATTNACTAAATANAANACTTCTCCCTCCTTTNTCCCANACNATTNCNTNNNTTTTGGTNCCCCCCNNTT
GNANANCACTCTNGNCNCCNTCCCNAGANTNCACNNAANNNTTCTCCNGATTTTNTAACTAATGNNGG
NNNCNATNNCNCAGGTTNTTANCTCTTTNNANAAANTCCNTNCTTNTNNCNNNNTNCGCAANTGGNNT
NTNCNTCTTNCCTCCCCCTAATCTACCATNTNATTNCTCCCCNTNNTCTTNNTNTGNTNNNTNCACTCA
NCNNANATAACNTCCACCTAANNTACNTCTANTACCCCNCTGTCNACNTTCNCACTCACNCACAAGTA
GTTNTNNGACNTGTATCTTCCCTCTCNCNNAGNACGNNCTTNTCGTNGAGAAAAANNATNNTCANCGN
CNAACTACTNNGTCNNCGNTCTGTACGTAANCTCNTCCTACGACTNNNGGNGNTAGTATTCNNTCNAN
GTCGTATNTCCCACCTCNTNATTATGCNTNGCNGNTNCANTANCNCTNTCTNANTNNNNNGATGGNNT
NGTTNCTNTNTCCATTNTNNNTNNGNATNNAGTGNACGANNGNTGTATTNCCTNTATCNNCTCTTCTCG
CTACNGNGGTTCGTNCNNTNNGNTCTNCANTCGNNANNATGTCGTACGTCTNATGCANNCNTNGCNGG
NTANCATCTNCTTATNTANNNTNNCATGATCNATNTTTTNATTNTCGTATNNGCTCNCCNACAGGCANG
NTNTCTCCTATGAATCNGTTTCTGATNTATGCTNCGTNCCNAAGTAGATNCNATGTCNCGG
TTNANNAAANNCTGGAGCTCCACCGCGGTGGCGGCCGCTCTAGAACTAGTGGATCCCCCGGGCTGCAG
GAATTCGGCACGAGGNNAGCGTCAAGATNTGTNACTTCNNCNNGGATCANNCCGNANNGTTCAAGGA
GGNCTTCCTNCTGNTTNACCGAACGGGGNATGNCATNATCCTGTNCAGNCNGNGTGGGGATGNGANN
NGGNCCCTGGGCCATANTTCCACNANNNCCNNGGTGNTCNAAGTNTTGGGGAACCCNAAAAGTANTG
AACATTANCNNNAANNGACCTGAATTTTGGCNNTTTCCAACCCNTCTTGTAAATTNGNGCNANAACTA
GGNACAANGCCNCTNNCANGNCTNTGGGCNATGGCNNCNGGTGNTNNGCNAAGGAAGGAACCGGCC
TNACNNNGGANNNTAAAATCCGCTCGGTGNCCGNCCNNNNGGTTANNACATTACCNNTGGACAACTT
AAAGNTCCCNNGTNGCTTGGGCCTTAAGGCCTTTTANTGNTNGAANAANTANTAAAAACCTNGTCCGN
TGGGGCCCNAATNGCNTNANGANTTTTCCAAATGNGCCTNGACACCNTGCCCTTTCTTTGGGGGAAAA
TTNGTTTTNTGCCCCGACNCTTTTNAGNTTTNTTTGGGACAANNTNTTTTTCCCCAAATTTNTTTTTTTTT
GGANGAAGNNTTNGCCCNTNNACCNTTCCCCCAAATNAAACTTTNGTNTNNTTTGTTTTTTAAAAAAA
AAAAAAAAAAAAAAAAAAAAACCCNGNTTTTGGGGGGNCCCCNCNTNNNNCNNNNNNNNNNATTTT
NCCNAAAAANCCCANAANNNNNNNNNTNNGNCCCTNNNNNCNNCCNTTNTAANGGGGGGGGNCCCT
NGGGGNCNCCNTNNNNNTTTTTNTTNGGGGNNNNANANNNNNNTNGGGGGNNNTNGGCCCCCCCNCN
TTTTNNNGGGGNNCCNNTTTNNNNNNAANTNNNTTTTTTNNNNCCCNNNATNTTT
NAAAAGCTGGAGCTCCCCGCGGTGGCGGCCGCTCTAGAACTAGTGGATCCCCGGGCTGCAGGGAGAG
TCTGAGGCCAACGTGAGGGAAATCTTCGACAAGGCACGCCAAGCTGCCCCCTGTGTACTGTTCTTTGA
TGAGCTGGATTCGATTGCCAAGGCCCGTGGTGGCAACATTGGAGATGGTGGTGGGGCTGCCGACCGAG
TCATCAACCAGATCCTGACAGAAATGGATGGCATGTCCACAAAGAAAAATGTATTTATCATTGGCGCT
ACTAACCGACCTGACATCATTGATCCTGCCATCCTGCGACCTGGCCGCCTTGATCAGCTCATCTATATC
CCGCTTCCCGATGAGAAGTCCCGTGTTGCCATTCTCAAGGCCAACCTGCGCAAGTCCCCAGTTGCCAA
GGATGTGGATTTAGAGTTCCTGGCTAAAATGACTAATGGCTTCTCTGGAGCTGACTTGACCGAGATTTG
CCAACGTGCTTGCAATTGGCCATTCGTGAATCGATCGAGAGTGAAATTAGGCGAGAACGGGAGCGG
CAAACCAACCCGTCAGCAATGGAAAGTAGAAGANGATGATCCAGTGCCTGAGATGCGCCGAGATCAC
TTTGANGAAGCTATGCCCTTTGCCCCGCCGTTCTGTCAGTGATAATGACATCCGGAAAGTATGAAATG
GTTTGCTCANACCCTTCAACAAANTCNANGCTTTTGGCAACNTTCANAATTCCCTTTAAGGAAACCAN
GGGNGGAGCTGGNCCCCAGTCANGGCAATGGCGGNGGCACCAGGTNGCAATGTGTTCGCCNGAAAAA
TAATGGATGATGANCCTGTNTGGNTTAAANGGGGGGGGGNCNACCAAGCAANGAACCTGGCCTGGGT
TGGACCNTTTTTTCCCTTGNNGGGGGGGGGCCCCCCCCCCAGGAAGGGAACCCGGCCCTNNCT
AAAAGCTGGAGCTCCACCGCGGTGGCGGCCGCTCTAGAACTAGTGGATCCCCCGGGCTGCAGGAATTC
GGCACGAGGCTNTGAAGAGGTTGGGAAGTGACCACTTGGATGTCTGGGCCCTGCCAAGGGGACAGGG
AGGGTCAGAGGGAGGCCAGCTGCTTCCTGCCCCCACCCTTTCCCCAGGCCCACTGTGCTGCTTTTGTGC
CAAGATTAAGGTTAGCCAGTCCCTTCCCGTCTTGTTCTGTGTGCTTCCGCCTACCCCTCATCACCGCCAT
GCCCCTGCCCCACACCTCCCCAAAGAATTGAAATGTCAGCTCAGGATATGGGGCCAATTTATGTGAAT
CCAGCATGTCCCTGTGTCGGCCTAGTGTCCCTTCAACCCGGGCCAGCCAGAGCCCCCCTTGTCGCAAGG
CCTCGTCCTGTCGCCAACTTCTCGAGCACAATCGGGCTTCTCCCATCTCCAGGTGTTCTGCCGCTCTCA
ACCAGAGGTGCCTCTTCGAACTGGGGCGGGAGTCAAAACCTGCCCCCTCTGTGGGAGGGACGTGGAGC
CCCCTCAGCCCTCACTGAGTTCTTTGGAATTTGCTCTCAAGTGCCTTAANCAAAAGGAAAACCTTTGC
TTGGTGTATGGNGGAAGGTCCCTTGCCTTAAAACCTTCTTCCCCGGCTTTGTNCTTCCCCAAGNGCCTT
TCCNTTGGCCTTGCTGGAAACTTGGGGGTTTTCNCTTGTTTCCCCAANTCCCCACAAACACTTGGCAAA
AAAATTTTTTTATTTTNNTGNCCAATNGGGGNGGGGGGGTCAAAGCCCTTAAACCCTTNTTGGGGGCC
CTNGGGCCCTTGTTCCTTTNNTNTTTGGGGGCCCGCAATTTTTTTTTTTGGGGGGGGGGGGNGGGGNAA
AAAAAAGGGCCGGNNAAAACTTTTTNCCCAAACCCTTNTNANNTNAAANGGGGGGNNTTTTTTCNNCC
CCCCNCAANANNTTTTNAAAAAAAAAANCCCCNTTTTTNGNTTTTTTTTTTTGGGACNNT
TTTGGAANACNCATCTGGAGCTCCNCCGCGGTGGCGGCCGCTCTAGAACTAGTGGATCCCCCGGGCTG
CAGGTGCAAGTCTTTTAATAGATGTGATANCAGTGTTTNNAGAACTCACTGATNNGGATGCNAAACAC
ANAAGGTNNTCCCCNNNNNATTNCATAAANTATTCTTTNTNTGTCNNNCCGACNNCNNNCTCACACCN
TNGAGNATANACTTTTTTCTGTNTATCTACACNCNNNGTNCTTATCTTTCAAATTNAAGTNATTANCTA
TTATNNNNACCCCNTTCNGTGACCTNANNTGTNTTTCGACAACNCCNCNCCNTTTCNTNTCANTNCACC
ACCATNTTANTCCATTCTTTNTGCNTGNCATTATNCCNCNNANCCTCTNTGGTTNCTCNTTTANACTGC
NNTNTTCNTNNNCNTTCNTNCNATGTTNTCNNNNTCTNNTNNCTANNTTTCCNNNTNTGCNCANTCTNT
NNTTCCTTTTATTTTACGTAATNNNNTTNTNNNTCTTNCATNTCNATCCCTCTTATATNTCNTATNACAC
TCANNTNCNTNTNTTAACNGCNTNCTNNCTGNNNCCTCANCTNNGCTATATTTCTATNANACATTCNNC
TANANTNNNATATCCCTAATTCNACNCTNNNTCAAANTCNAGCNNCACCNCTCCNNNTTCANACCTTT
TCNNANTTTNCNCNCCAANNNTNANCTNTNTCTCACCNNNTATTNNCNCACTACNTNTCCCNCTTTNCN
TTACACCANTTAGANNACTCTNTTTNCATNCACAATTANNACNTTCTATCTTCTCTATANANNTNNNNC
CTTTTNNCCTCTANATTCGGTGAANCGNCAANANTATTTATNANGAGATNAATTNTCGTGCTAACNCTC
ACNNTCTTTATAATCTTANTCNACCTTNTTCNTNNNTATCNTATNNTNTTCTCTTTCGCTCTCNTTNCTC
NTCCNTAGNTTATTTTCTCTCTTCTTNNTCAANATCNGNACNGNNTNCATTAATATTCNATTCANTACC
ATCCTACTTGTNCCNCNTAGTNTTNCNCCNAAGTCNTCATATNTNANAATGAATANTGTGTNATTTGT
ATCACTCTTTAACTNTAGCCCG
NAAACTGGAGCTCCACCGCGGTGGCGGCCGCTCTAGAACTAGTGGATCCCCCGGGCTGCAGGATTTGA
ACCACTAGCCTTTGAAATAAAAGTCAGCCCCGTTATTAACAGTGCAGATACTGAGTCCACTTGGATTTC
TTGCAGCTTTCTTTGCACTGCTTTAGGCAAAAAGTGTTCATCTGGTTTTTTTCATTTGACACCCCAGTTT
GCTGCAGTGACAGAACTTAATGCCTAATCTGTAGAGTTCATATATATATATATAAAATATATATACCCA
CACAAACACATATATATATGAAGTTTAATGAACTGTCCAAGTGAAACATCCTAACTGTCCTTGTGATTA
AGGAGCCAGTTTTGCAGGCAGCCAGCGCGGTGTTTTTCTGTTGGTCTCCCAGCACATGCCCATTCCTTT
GCCCGCCCATTCCTGCTACACTCGATGGCAGCTGGTGACACCACTGTGAAGGTTTCTGCGGTCCTGTCT
TGAGTGTGGCAGGCTGGAGGTGTACTGCCAGAGGTGAGCTGGGGCGTGTTCTGTTCCCCACACATTTC
TAAACTGCTACAGGCAGTGTACTGGCTACCACCTCAAGTCCAGACACAAGTCTAAAGCTGAATTTTGT
GTCAGTAACCTTAACCCACCGAAACAAATCTTGAGTAGCTCGTATCCTGCTCTTANGAATTCTGNGTC
ATTTAAAAGTAATGTTCAACTTTCTTTTCTTTTTTATTGTTTAACCTAAGCACTGTTTACAGGTGTATGC
TAAAGCCTGAAAATTTTGTCTGGNGCTGNGGNGTATGNANCATTGGCCAATTTTTATATTTAATTGGCA
NNCAAAAAAGAAACCTTAAAGANTTTNTGAAAAAATGAAGGAAGCGNTGGGCTTTGGCTCCCTAAAT
CTGGGNGGGGTGCCTTGGNGGGAAAAAAGGGAAATTGGGGGTCTCTTTNAAAAAGGAAGCCTTTTTG
GGGGGNGGGGCACCCCAGGCTTTNNCCTGGGGNNNCCTGNTNGGG
GCCTATNGGAGCNGNATNACAGTGGCGGCCGCTCTAGAACTAGTGGATCCCCCGGGCTGCAGGAAT
TCGGCACGAGGCCGCGTTCGGACCTCGCTGCTTCAGCCTCCGGGTCCCTTTCCATCTTTCCACCCTGCG
ACTAGCAGCANANAAAAACTAATTCCATAATATTTTTTTGCCCCGTATACACTTTGAGGCGAGCAAAA
AGATTANAATAAATTTTAACAATGAGGGAAATCGNGCACATCCAGGCCGGNCAGGGNGGCAATCATAAA
TCGGTGCCAAGTTCTGGGAGGAGATCANGAGATGAACATGGTATNNACCCCNCCGGCNCCTATNNTG
GGGACAGCGACCTACAANNGGACCGNATCTCCGTGTACTACAATGAACCCACNGGNGGCAAATATGN
CCCTCGTGCTATGNTGGGGGATCTAGAACCCNGAACCNTGGACTCTGTTCGNTCAGGTTNTTNTGGNN
AGATCTNCACANCANACNTCTTTNTTTTTGGTCAGGGNGGGGCANGCAACNGGNGNGCGNGCGGGCT
ATTTATAANGAAGNAGCCCCTGGTTGANTGACGTCNCTNGATGGNTGTNGNANGGANGGNGNCANGA
TAGNNTTTGNCTGCCTGGCTTTNCTTTCAACCCACCNNCTTGGTGGGGGGGCCTGCCTCGGAAANGG
GAACCTTNCTCTTTCTNATNAAACCCGANAAANNGATTCTNNTCCCGGAANTGNAAAAACCTTCNCNN
NGGNGGNGNCCCCCCCNAAAANATTNNANNACTNNGNNGNNTGNGNCCTTAAANNCCNCCNCCCTTT
TTTNTTTATTATTGGNNGGGGGNANANNNTTNNANAATATCCTTTTTTNTTTTNAAAAANAGGCNTNTT
TTNAAAAATNNATTTTTTTNACCNTTNAAGGTNGGCCCCCCCACCCTNTTGGGNGNTTTGGNCGCCNTN
TNTTNNCCCCNCATGGNNNGGNGNNCCCCTNTTTCCTNCCTTTTTTCTTGGGNGNNANNANTTNGTNCC
CCCCC
GCCCTATANGGAGNTGNATNACAGTGGCGGCCGCTCTAGAACTAGTGGATCCCCCGGGCTGCAGGAN
TNGGNCTCGAAGATNTNAAGCCATGCCTTNTCCTGTACNACCANATNNTTATGNTTGANCTTCNNN
CTCCNGNTTNANANNNAGAGNANAAAGATGNAAAGGATGGCNNGGNGCNNTGNCNGGNGNCNNNGNGC
NAATNTNAACTTAAACCNAATNAATTATGGGGGNGGTAAATACAGACATTTTAACCCAAGCCACNAN
NNTNNTANNNGGNACNCAGCTTTTACGGNCNCNTGCTCNCCCANNAATNGCNCCGNTNTGCTTNTGCC
GNNNNCNCCTCCANCCATTGNCTTGGCNNTNCATNNGCGGAACNCNATGATTNCNGGCCGTCTGTGCT
ATNNNNNCCNNTTGGNTGGCTNGGANTTTCTACTGTTNTNTTCNNAANTNANATNNACNACNTCAGNC
TCTGCGGGTAATTCTATNTNAGGGGNNNTGGCTCGNCNCANAGGCACACCTCCTAANNTGGNGCNGN
GCAAAACCCCACGGGGGNAANNTTTCACTGNNCATCNNNTGGNTNNTGTAATGNNTTCNNCCCNGGG
GGNGTTTATTGAGCACNCCNNGGNGANNANNNTTNCNGATATTCTCCCCCCCNGGTNAGNCNNTGNG
CGAACCCNNAAATNTTTTNCCTANAANGCCNNTNNTTCGATACTAAAAAAAANAATGGGGGNNGNNN
TTTNATATATGCCNNTTNACCCCTCNCATATNNNNNCCCCNTNTTTAANNATAATNGGCCTCNTTTTAT
TTTAGNNGAGGCGGCTTNNTTTTTGTNNNNAGGGAANNTTTNGAAAAAATTTTNNCAAAAAAAATTGN
NCCCANTNTGTTTTTTTGAAAACCCCCNCCNNCTTTTNTCANATNTNGTNATAANCNACCTTCCTTTTA
NAGNCTTGNTTCGCCCCCCAAANATTTCTTTTANGGAATATTNNTGNAATGAAAAGNGGTC
GCCTATAGNGAGTCGTATTACAGTGGNGGCCGCTCTAGAACTAGTGGATCCGCCGGGCTGCAGGCNCG
AGGCCTGGGACAGGCCAGTCCCGTTCCTTCCGCATNCTCGCCCACCTNACAGGCACCGAGTTCATGCA
AGAACCCGGATGAGGAGCACNTGAANAATTNAANCCAGGTGCCNAGGACGGAANCCCNACCCCAAG
CNTAANNTAAACCCAAGNACCCNTGNCNNGGGCTNACAACCCCAANNCCAANAACCNGCCNNCCTTG
GNGGGGGNNCCTTNNNTTNNNNNACCCNNACCCCAAAACANAACNANANGGTGNTNAACCGGNANA
NACAAATCANGCACACACCAAGCGTATGCAAAANACCGACCTTNATTTCGCAAGCCGCNCTTTANAG
GGGNGCCTNTGGGGGGGNGGGGGGCACACAAAACGAAAAAAACTCGNTGTGCCACCCNAGGCCAAA
GGGNTNNCNNGGTGNCTTATNTNTTGNGGCTTNTGCCNNTGCATTATNCNNCCTGNAGAAGTTTTTGN
NCTCCCCACAAGTTGGGAAAAANNTTTNGAANAAAAANGGGNCGTTTTTTTTNAGANGAAAANGGGG
NCTTTTTTTNTCCNCCACNTNNTNANAATGATGNGNNNNTTATNCCAACTTTTTGTNNNNTGTCAANTA
AAAAAAAANTACNTGGTTGGAGGGNATTNACTCCCCCNTCNTNAAANAAANNATGNGNCANAGGGTT
TTGTTNTTTTNNTNCTGCNNCANTGCANAAGNGTNCCCNNTTTTTGANAANNNNTCTTTTTTTTTTTNA
GGGAGGNGGNGGCCCTTNNTTTNNNNGCCAGATTTATTNTTAAGATNTTTTGGTCNAAAAAANCTCNG
ANNGTGNTGTNTTTTTNTAAATANANNTTTTGGNGNACGNANTTTNNTTNAATNAAATNTNTTGTTTTT
TTTTGTTNTAGAATNATNANTTTTGTTTTNCGCGNCANTTTATTNATANNANANCCAGNGNTGNAAAA
NAAAATTNTTNN
GCCTATAGNGAGTCGTATTACAGTGGNGGCCGCTCTAGAACTAGTGGATCCCCCGGGCTGCAGGCNCG
AGGCCTGGGACAGGCCAGTCCCGTTCCTTCCGCATNCTCGCCCACCTNACAGGCACCGAGTTCATGGA
AGAACCCGGATGAGGAGCACNTGAANAATTNAANGCAGGTGCCNAGGACGGAANCCCNACCCCAAG
CNTAANNTAAACCCAAGNACCCNTGNCNNGGCCTNACAACCCCAANNCCAANAACCNGCCNNCCTTG
GNGGGGGNNCCTTNNNNNTTNNNNNACCCNNACCCCAAAACANAACNANANGGTGNTNAACCGGNANA
NACAAATCANGCACACACCAAGCGTATGCAAAANACCGACCTTNATTTCGCAAGCCGCNCTTTANAG
GGGNGCCTNTGGGGGGGNGGGGGGCACACAAAACGAAAAAAACTCCNTGTGCCACCGNAGGCCAAA
GGGNTNNCNNGGTGNCTTATNTNTTGNGGGTTNTGCCNNTGGATTATNCNNCCTGNAGAAGTTTTTGN
NCTCCCCACAAGTTGGGAAAAANNTTTNGAANAAAAANGGGNCGTTTTTTTTNAGANGAAAANGGGG
NCTTTTTTTNTCCNCCACNTNNTNANAATGATGNGNNNNTTATNCCAACTTTTTGTNNNNTGTCAANTA
AAAAAAAANTACNTGGTTGGAGGGNATTNACTCCCGCNTCNTNAAANAAANNATGNGNCANAGGGTT
TTGTTNTTTTNNTNCTGCNNCANTGCANAAGNGTNCCCNNTTTTTGANAANNNNTCTTTTTTTTTTTNA
GGGAGGNGGNGGCCCTTNNTTTNNNNGCCAGATTTATTNTTAAGATNTTTTGGTCNAAAAAANCTCNG
ANNGTGNTGTNTTTTNTAAATANANNTTTTGGNGNACGNANTTTNNTTNAATNAAATNTNTTGTTTTT
TTTTGTTNTAGAATNATNANTTTTGTTTTNCGCGNCANTTTATTNATANNANANCCAGNGNTGNAAAA
NAAAATTNTTNN
TTNANCAAANTCTGGAGCTCCACCGCGGTGGCGGCCGCTCTAGAACTAGTGGATGCCCCGGGCTGCAG
GAATTCGGGACGAGGGATGGTACAATCAAGGAAGTCGTTCTGGATCCAGACACCACCTCTTACAGCCT
GACGGATCTGAGCCCATCCAGCTACTACACAGCCAGGATTCAGGCACTGAACGGGACCCTAAGGAGC
AAGACAGTCAAGACCNTCTTCACCACGAGTGGAGTCCTGTACCGATTCCCCAGGGACTGCTCCCAGGC
AATGGTGAATGGAGACACGACCTCTGGCGGCTACACCATTTATCTGAACAACGACAAGACCCANAAG
CAGGAAGTCTTCTGTGACATGACCTCTGACNGGGGTGGATGGATTGNGTTCCTGANACGCAAAAATGG
ACGTGANGACTTNTATTCGAACTGGNAAGGCTACCCTTNTTGANTTGGGACNTNAAAAAANAAGTTTT
TGCNTTGGCTTGGACACTTTGNACCAAAATNCCAGCCCAGGGCCATACCAACCTCNGGTGGANCTTGC
GGAACCCCGGGGAAGNCGGCCCAGNCCTTTTANAAAAAAGTTAANGTNGGGAAAAGCCCGGACCCCC
TTNCCGGTTNAAGGNGGANGGGGTCCCANNGGCCCGGGAGGNGACCTCCTTTGGCCTTACCCAATNGG
NNNAATCCTTTTTCCNCNTTTAAAAAGGGACAANNAATTNGGCCATTAACCAANNTTGGGCCTTTTCN
TTAAANGGGGGCNTTTTTTGNTNCAAAAANCTGCCCCCCGGTTAAACCTGGGNGGGGGAAAANCNGG
GGAAAACCCCCCCCAANNNNGGGGGGTTAACCNGGGTTCCCCTTGGAANGGGGCCCCAAACNTTTTA
ATTCCANTTTTNTTTAAAAANAAANCTTTAAAACCCCCNNANTTTTTCNAAAACCTTTTAAAGGGGNG
GGCCCAAAAANGGGCTTAAANTTTTCCG
NCAAGCTGGAGCTCCCCGCGGTGGCGGCCGCTGTAGAACTAGTGGATCCCCGGGCTGGAGGNGAAAA
GCCNTGNNATGANGAATGGAANAGAGTGGATTGGGAGCAATTATNCTTTTGNCTGTCAAAATGAGTG
CCTTTTTAGGAAATGTGGTTCCCAATTGTTGGTTTCCTGCCATCGTGTGTTCTAAGGAAAATTATCCAT
GGCTCATTAAAAAAAAAGTGCTTTATAGCACTGAGATAAAATTTTACATGGATTAAAGTGATTGAG
TATTTTAATCGNAATTCGTCCAAAATCAATATCACTGTGTATTTCTGAGCATTTTCAAGTCTTTTANTTA
CATTCAGCAAATTAATAATGATATAATAAATTTAAACAATAGACATTGGCGTGGTCTTTTTCAAAAGT
ACTTAAGATTTGATTTATGGCTCTTACCCAAGAATCATCATGAAAAATCGATTTGTTCTGTAGCAGAAC
TCAGTAAGCATGTCTTATTTCAAATGGCATTTTATTTTAAAGACAGAACCACTTAATCTTGGAATAT
TGTTTCTTCAGTAAAGATTTCTGAAATTTCCATTTATTGGTCTTCTTTTCCCCAAACCTAATCATCCCAC
CTCATTCCAGATGTCCTACTTATGTTTTTCAAAAATGTACTTGAAAACTAATTGCCCCATCATGNGGGT
ATCAAATTTAAATAAGTGATTCTCTGNGGCCAGTTGTCTTTCTGGAACTTGGNGGGTAATGGAAANCCTG
GATCTGCCAAGAAACTTTAAAGGGAAAGGGAGTAACTGGGTTCTACTCCCCATGACCGGGGNCNGGG
ATTTCNTTTGGTNGAAAATCAAAATCATAAGTTGGATTCNCTTAAATAAAGCCCCCAGGATTAANGG
GGTTAAAAATTCAATCTTTTAAATCCCTCCTTNCCAAAGTNAAAA
TTNNNNCAACAAGCTGGAGCTCCACCGCGGTGGCGGCCGCTCTAGAACTAGTGGATCCCCCGGGCTGC
AGGNNANNNGCCCNGNNATGNAAAAAGGAAAAGAGNGGANNGNGAGCAATTAGGCTTNTGNCTGTC
ANAATGAGNGCCTTTTTAGGAAATGTGGTTCCCAATNGNTGGCTTNCTGCCATCGTGTGNTCTAAGGA
AAATTATCCATGGCTCATTAAAAAAAAAGTGCTTTATAGCACTGANATAAAANNTTACATGGANCAAN
GGGATTTGGAGTATTCNAATCGCAGTNCGNCCAAAATCAATANCACTGTGTATTTCTGAGCATTTNCA
AGTCTTTTAGTTNCATNCAGCAAATGAATAGTGATCTAATAAATTAAANCAATNNACNTTGGCGTGGN
CTTTTTCAAAAGANCTTAANATTGGNTTNATGGCTCTAACCCAAGAATNATCATGAAAAATCCATTTGT
TCTGNAGCAGACCTCANTAAGCATCTTNTTATTTCAAANGGCAATTTTATTTTAAGGACAGACGCCCTT
AATCTTGGAATATTNGTTCTTCAGNAAGNATTCTTGAAATTTCCATTNATNGGCCTTTTTTCCCCAACC
CTAATGATCCCAACCTCAATTCCANANGTCCTACTTAATGTTTTTCAAAAAANGTACTTGAAAAACTAA
TTGGCCACATCATGNGGGNAATNAAAATTAAAATAAGGGGATTTCTTNGGGGGCCCAGTTGGTNTTTT
GGAACCTTTGNGGGGNNAATGGGAANCCCTGGGATCCTTGCCCAAAAAACCTTTTAAANGGGGAAAGG
GGAGTNAACCNGGGGTTCCCANCNCCCCCCAAGGACCGGGGGCCNGGGTNTTTCCTTTGGGNGGAAA
AATCCAAAACCTTTGGTTGGAATTTCCCCTAAATAAGGCCCGGGAGGAATTAACGGGGTNAAAAATTC
CAATCTTTTAAAACCCCCCCTNCCCAAGGNNAAAAAAGCCTAAANGGN
TTNNGCACANCTGGAGCTCCCCGCGGTGGCGGCCGCTCTAGAACTAGTGGATCCCCCGGGCTGCAGGT
GCAAGTCTTTTAATAGATGTCATAACAGTGTTTGGAGAACTCACTGATGAAAATGTGAAACACAGAAA
GTATGCAAGGTGGAAGGCCACATATATTCATAATTGTTTAAAGAATGGAGAGACTCCTCAAGCAGGGC
CTGTTGGGATTGAAGAAGATAATGTAGATGTTGAGGAAAATGAAGATGCTGGAGCCACCTCTCTGCCC
ACTCAGCCACTTCAGCCGTCATCTTCCACATATGACGCAAGGAACATGCCATCGAGCAGCTATACTGG
AATCCAGATTCCCCCCGGTGCCCACGCTCCAGTAATACCCCAGCAGAAGTGCCTCACAGCACAGGTGT
AACAAGTAACACTATCCAGCCTACTCCACAGACTATTCCGGCCATTGATCCTGCAGTCTTCAGTACAGT
TTCCCAGGGAGATATCCGACTAACACCAGAGGACTTTGCTAGGGCTCAGAAATACTGCAAGTATGCTG
GCAGTGCTTTGCAGTATGAAGATGTCAGCACTGCAGTGCAGAATCTGCAGAAGGCCCTCAAGTTACTG
ACTACAGGCAGAGAATGAAGCCTTTGTCAACATCCATGCATTTTTTGGCTTAAAGAACTAACAGTCCA
CTACTCTATCTTCAGCCTATCAGGAGCACAGNTTTTAAGGAAGAACTTCAGTTNCATTGACTTTTAACC
AATGAAAACTGGGGCTGGGGATCAAAATTCCTATTGAAGTATTCATTAGCAGGCCTCAAACCAGNTTT
CANTGGGCCATTTAANGGGATCCNGGAAGNCCCTTAAGGGGATAAGCTTNTNTAATGGGGAACCANG
GNCCCTTTTATTTGGCCCNC
GCCTATCTGGAGCTNCACCGCNGTGGCGGCCGCTCTAGAACTAGTGGATCCCCCGGGCTGCAGGAATT
CGGCACGAGGCAGCCGCATCCCTGAGACAAGATGGTGAAGGTCGGAGTGAACGGATTCGGCCGCATC
GGGCGCCTGGTCACCAGGGCTGCTTTTAATTCTGGCAAAGTGGACATCGTCGCCATCAATGACCCCTTC
ATTGACCTTCACTACATGGTCTACATGTTCCAGTATGATTCCACCCACGGCAAGTTCAACGGCACAGTC
AAGGCAGAGAACGGGAAGCTCGTCATCAATGGAAAGGCCATCACCATGTTCCAGGAGCGAGATCGTG
CCAACATCAAGTGGGGTGATGCTGGTGCTGAGTATGTGGTGGAGTCCACTGGGGTCTTCACTACCATG
GAGAAGGCTGGGGCTCAGTTGAAGGGTGGCGCCAAGAGGGTCATCATCTCTGCACCTTCTGCCGATGC
CCCCATGTTTGTGATGGGCGTGAACCACGAGAAGTATAACAACACCCTCAAGATTGTCAGCAATGCCT
CCTGCACCACCAACTGCTTGGCCCCCCTGGCCAAGGTCATCCATGACCACTTTGGCATCGTGGAGGGA
CTTATGACCACTGTCCACGCCATCACTTGCCACCCANNAAGACTGTGGATGGCCCCTCCGGGGAAGCT
GTGGCNGTGACGGCCGAAGGGCTTGCCCAGAATATCATTCCCTGCTTCTACTGGCGCTTGCAAAGGCC
GTGGGCAAGGTCATTCCTTGAGCTCAACCGGGAAACTTCACNTGGCATGGGCCTTTCCNGTTCCCCACT
TCCAACGTGTCTGTTTGTGGATNTGAACCTNGCCGCCTTGGANNAAACCCTGGCCAAGTTTTGAATGA
AGANCCAANAAAGGNNGGCGAAACCNNGCNGTCNNAAGGGCCCTCTTCAAGGGCAATTTCTNAGGCT
TNCCCNNGAGGGACCCAGTTNNTNTCCCTNGGAATTTCAANAGAGAGAACCCTTNNTTTTTTTANCCTT
CCG
AAAACTGGAGCTCCCCGCGGTGGCGGCCGCTCTAGAACTAGTGGATCCCCCGGGCTGCAGGTTCTTAA
TTATCACAGCAAATACTTTGGTCTGATTGTCTGTATCTTGTGACAAGCGATAATGACCCAGTANAATTG
CATCCGTTCTGGTATTCCTGGNGCGTAAGCGTGGAACAATGGACTGGGGCTCCTCGGGCGTGGTGAGC
ATCATCACGTGGCCATCCGGGAAGAACGTTACGTACCTGTAATATTTCCACTTGGTGCCACGCTCTATAN
AAACCATCAAGANACTGCTCCCCTTGACAAATATAGGNGGTTTTACTGATATACACGCCATCAAATCG
AACACGGGGCCTTTCCAAAAACATCTCTCTCCANGATGTGTACGGAACAAGTTTAATACAGNTTCTGC
CCCAANCTTTCAAGCAGGCCAAACGCCNTATTTGAGGGTNTCTGGCANANATANANAATCCTCTGCAC
ACCTGTGACANCTGCTCTAATGATCTGANGTNCAGGTCGNTGCAGACCACCCAACGNAAGACGTACAT
CACGACCTCCATTGGCAGANCTTGATATATGAGTCNGACTGCTCTCAAGTTCAGGCTGACACAGNTNG
AGCACAGACTTCTGAAACGTGAGCTGCTGTTGGAAGTAAGACANGANGTCTGNCATCTTGCTGCATCA
TCAGTATCTTCAATGNAGCTGGTTCCCACGCCNTCACCATTTGGAGACCGGGTATAAAGTAATCTTGA
ACTCTATATCAGGGACAAGTTTGCATANCCCCTACAANAAAACTTGGATGGGTTNATAAAAAAGCTCCA
TTTGGTCTTGNTCNACCGGNTTTTAAGAAAAAATTTTGGANGGGGGGGGCCCGGTACCCAATTTNGCC
CCTTTAGNGGANNNANNNNNNNAAANNNNNNNNNNNNNNNNNNNNNNNNNNNNNNNNNNNNNNN
NNNNNNNNNNNNNNNNNNNNNNNNNNNNC
GCCCTATNTGGAGNTGNATNNCAGTGGCGGCCGCTCTAGAACTAGTGGATCCCCCGGGCTGCAGGAAT
TCGGCACGAGGCAAATTCCTTGCAGCAGGAACCCACTTAGGTGGCACCAACCTTGANCTTCCAAATGG
GAACAAGTACAATCTANCAAAAGGGAAAAGTGATGGGATTCTANATCATAAATCTGAAGAAGANCGN
GGGAAAAGCTTCTGGTGGNCGGTTGGGCCATTGGCGNCATTGAAAANCCGGNTNNNGGCAANGGCAT
ANTCNTCAAGAATNNTNGNCNANGANCTNGGNTNAAATTTGNTNGTGGCNNTGGAACCNNTTCTANN
GNTNGNCGGTTNANTTCGGGAACCTTTACTNAGCAAANNCNNGNCNNATTNAAGGANCCAANGNTTN
TGGNGGNCACCGATCCCANGGCTNANCANCAACCCCTTACGGAANCCTTTTANNTGAACCTGNCAACC
ATTGGCCTGTGCAACANGGACTNTTCTCTGGGCTTCGTGGACATTGGCATTCCGTGCAACAACAANGG
AGCGCACTCAGTGGGTCTGATGTGGTGGATGCNCTCCCCGGGGAANTCCTGCGCATGCGTGGGNACCA
NTTTTCCGAGAGCACNCCGNGGGGANGNGATGCNCGGAACTCTANTTNTNCAGAGATATCTTGNGGA
GAATTGAAAAAGGNANAAGCNCGGCNCCCANCTTGAGAAAAGCTGTGACCCAAAGGANGAGATTTCA
CNGGGGGAAAAGGACNTGGTTCCANNCCTNCANANNNTTACAGGTTNGCTCCTACCCTGNNGGNGGG
AAAACNNGNTCNTGAAAGNNGTGCAANTTGCCTTTCCNGGCCCANTTTANCCAGNNTCCCCACTGGAN
NAAACNNGGAGGGCTTCAACCGTTTCNNCTGAANNAATAGGGNNTNGNGNCCCCCCGACCCGCCCCA
NGTGNCCCNGGAANTGGGGTNGGAAAACCCCCCC
TNNANCCTATCTGGAGCTCCANCNCNGTGGCGGCCCGCTCTAGAACTAGTGGATCCCCCGGGCTGCAG
GCTCTGAAGGAAAGCAGAAAATAGTAGAAGGTGGAAATGGGCGAAAAGAGAATGGTCATTAGTAGCA
GGAGAAACACAGGACANGGATCCCATCTTATTGGGGTTCTCAGAGGACAGCAGGACANTTTTTCAGG
ACTAGTAGGTAGGGCAGNGTGANAANCCCNTCNTCCAAAAANANANAGGTAANCCTTACCCNTTACN
TNTACNGGTTNTGGNGCCCCCAGGNNCAGCCCNTTTTTNNTANCGCAAAAAATTAGGGNCCCAAANAA
GGGCACNCCCAGGGNCCAAAANGNTGGTATTATANTTAGNAGGGAGGNTGATGGNAAGNACCCAAAT
CNTAAAGGCCAGGTCNCCAANTTGAAACCAGGAAAGGGGGAGAGGAGATTAGGGAATCTCTGTGTNT
NGNGCACCCGGCTNTGGTCNCTCTTGTGGCAGTTTTTTTTAAAAANCNTTCTCTAGCTCTAGCCCNATA
ACNNTGTAANATNTNCCNACCCNCCAGACCNGTGAGAGCCCTNCCCAAANTNTTGGGGGGAGAAGNG
NGCTCTTTTCACATTTNAAGGGNCCNGNTNTTGTGANNANNTTCTTCTGAAGACTCTTNANTTCCTCTT
GTTTTCCCCCCANAGGGNCTTNTTGNAAAAAAAGGAGGACCCCCCCTCCCCCCANTATTAACCCCCCA
NAAGAAGAACNGGAAGANNCCTTCCTTNTTCCCCCNCCCNCCTCTTGCCNTAATTTTCCCCGCCTGGTC
TTGTTTTAAAAAAAACNNGGCAATTTATTTTTTNNAACGGCCTNGGAAGGGAAGTNCNNCCNACGNCC
CCTNTTTTTTTAANAAAAAAAATTNTTTTTCCCCCCCCCCCCCCCGTTGGGTTCCNTTTTTTTTTAAAGN
TNNANCCTATCTGGAGCTCCANCNCNGTGGCGGCCCGCTCTAGAACTAGTGGATCCCCCGGGCTGCAG
GCTCTGAAGGAAAGCAGAAAATAGTAGAAGGTGGAAATGGGCGAAAAGAGAATGCTCATTAGTAGCA
GGAGAAACACAGGACANGGATCCCATCTTATTGGGGTTCTCAGAGGACAGCAGGACANTTTTTCAGG
ACTAGTAGGTAGGGCAGNGTGANAANCCCNTCNTCCAAAAANANANAGGTAANCCTTTACCCNTTACN
TNTACNGGTTNTGGNGCCCCCAGGNNCAGCCCNTTTTTNNTANCCCAAAAAATTAGGGNCCCAAANAA
GGGCACNCCCAGGGNGCAAAANGNTGGTATTATANTTAGNAGGGAGGNTGATGGNAAGNACCCAAAT
CNTAAAGGCCAGGTCNCCAANTTGAAACCAGGAAAGGGGGAGAGGAGATTAGGGAATCTCTGTGTNT
NGNGCACCCGGCTNTGGTCNCTCTTGTGGCAGTTTTTTTTAAAAANCNTTCTCTAGCTCTAGCCCNATA
ACNNTGTAANATNTNCCNACCCNCCAGACCNGTGAGAGCCCTNCCCAAANTNTTGGGGGGAGAAGNG
NGCTCTTTTCACATTTNAAGGGNCCNGNTNTTGTGANNANNTTCTTCTGAAGACTCTTNANTTCCTCTT
GTTTTTCCCCCCANAGGGNCTTNTTGNAAAAAAAGGAGGACCCCCCCTCCCCCCANTATTAACCCCCCA
NAAGAAGAACNGGAAGANNCCTTCCTTNTTCCCCCNCCCNCCTCTTGCCNTAATTTTCCCCGCCTGGTC
TTGTTTTAAAAAAAACNNGCCAATTTATTTTTTNNAACGGCCTNGGAAGGGAAGTNCNNCCNACGNCC
CCTNTTTTTTTAANAAAAAAAATTNTTTTTCCCCCCCCCCCCCCCGTTGGGTTCCNTTTTTTTTTAAAGN
TTGANNAAANTCTGGAGCTCCACCGCGGTGGCGGCCGCTCTAGAACTAGTGGATCCCCCGGGCTGCAG
GAATTCGGNANGAGGCTTNNCNNTCCNGNNGACCTCGAGGNGGGGNCCCGGNGCCCNNTNNNGCATA
TANTNACTCGNATTACNTGTNNNTCTTTANNGNNTGTNACNTACTAGGGCGAATGGATGAANTGAAAG
TTGGAGGAAANGANCTATTNGACTTAANANGCTNTTNATGTATCNNAGCTGCTNTGANTGGANGTGCC
NATNNTNCGAAGAATTACACCTTTTATGANGGCCCAAAGACATATATTNTCANAAACTGNNNNTGACA
CAGNCACAGCGCTTCTGGACCTGTTTCAAAAATNTTAGAATATTTGNAATATATANGATNCGGTGTTTN
CGCTGGNTAAACGATANAGGCAGNTTNGTGGAGTNTGCATANANNAAANTNANNGGCCTCGGCCCNN
AAAANAAANCTNCCNTTTTTTTTTTACNNNCCCTTTCNCCCGCTTGGCTTCCCGATTTTTTANNTNAGG
NNTGGAANAATAATTTAAATGGCNCCCCTTTTAANGANTGCCNACAAATTTTTGAANGGCTTAGGCCN
AGTTTTCCCCCTNGGGCANNAACCCTTNCNTTTNNATTTNCCAGNNCCTNGNTTNCCAGGGGGNCATT
NTCTTTTTGGAAAGNAAANNGCCTTTNNCCCCCCTTANATGGTTTNNCCNTTANTTAATTGGNCCCAT
NTNNNTTTAAAAAAACCNGGGGNCCCCCCATGAANACCNGGCCCNTTTGTTTTNAATCCCCCCCTTTTT
TTNAGGGGAANNGGGGATTTCCAAAAANNNNNNNAACCCCCCCNNNAAAACCGTNTTCCNCCTCCCN
NTNNAAACCNNAGCNNNAATTGGTTTTTTTTNCCCCNNAAANAATNTTTTTTTTGNAAAANGGCCCTN
NGGGCNNCCANTCNACNTTNGNACCTTGGGNNCCANNGGGGGAAAAAAAACCCCCCNTTNTGGNGNN
ACCTNCT
NCNNANCTGGAGCTCCACCGCGGTGGCGGCCGCTCTAGAACTAGTGGATCCCCCGGGCTGCAGGAA
TTCGGCAGGAGGCTTNNTGGGGNCTGAGCCGAGGCGAGGCCTGACTGCTCCCGCCGNCCACAAGCTGC
TGTCAGNGGGCTCTGCACAGACAGGTANAGGGCACCNAATTCCGGAGCGCCTAGAGCGTGGACAAGC
TGTACCCGGAATCGNGGGGCGCGGACACCGCCTGGAGGGTCCCGGGCNACGCAAAGCAAGGCAACGA
TGACATTCCTGTNTATCGCTTGACGATATNTNACTGTCGGANCAGTGGTCCTGGGGGCCAGAACGTTA
ACAAANNGAACTCCAAGGCTGAAGTCAGGNTCCACCTGGCAAGCGCCGACTGNATCGCAGAGCCCGT
GCGGNAGAAGATGGCTCTCACGCACAAAAATAAGATCAACAGGGCGGGAGAGCTGATCCTCACGTCT
GAATACAGCCGCTATGAGTTCCGAAACCTGGCAGATTGCCTNCAGAAAATTCGAGACATGATTGCCGA
GGCCAGCCAGCCAGCCACAGAGCCATNCAAAGAAGATGCTTGCCTTCAGAAACTCANGATAGAAAAC
ATTGAATCGCGAAANGGTTCCGAAAAAAAGANAATAAACTTTTGNCATTNAANACGAGCAGGAGGGT
CGGTAGCGGACTTGAAAGCACCCCTTCGAGCCAGCAAAANCCGCTTCANGGCCNCCCAGGTGGGTTCG
GACACCAAGAAAACGGGNGTCTGGTCCGGGGCACTTGGTCGNNTTTTACCGTNGGAANCCGTNGNAA
AAAAGGTCCTTTCGGGAACCNAAGGTTTGTAAGNCCCTGGGCCNCCCAAGGTNNTTTTAAAGGGGAA
AACCCGNGTTTTTNCCANCCTTTCGTTTTNCCATTNGNCCCGGNNTTAANTTGGCAATNAAAAATTNTT
NAACTTCNAAAAAAAAAT
NCANNANCTGGAGCTCCACCGCGGTGGCGGCCGCTCTAGAACTAGTGGATCCCCCGGGCTGCAGGAA
TTCGGCACGAGGCTTNNTGGGGNCTGAGCCGAGCCGAGGCCTGACTGCTCCCGCCGNCCACAAGCTGC
TGTCACNGGGCTCTGCACAGACAGGTANAGGGCAGCNAATTCCGGAGCGCCTACAGCCTGGACAAGC
TGTACCCGGAATCGNGGGGCGCGGACACCGCCTGGAGGGTCCCGGGCNAGGCAAAGGAAGGGAACGA
TGACATTCCTGTNTATCGCTTGACGATATNTNACTGTCGGANCAGTGGTCCTGGGGGCCAGAACGTTA
ACAAANNGAACTCCAAGGCTGAAGTCAGGNTCCACCTGGCAAGCGCCGACTGNATCGCAGAGCCCGT
GCGGNAGAAGATGGCTCTCACGCACAAAAATAAGATCAACAGGGCGGGAGAGCTGATCCTCACGTCT
GAATACAGCCGCTATCAGTTCCGAAACCTGGCAGATTGCCTNCAGAAAATTCGAGACATGATTGCCGA
GGCCAGCCAGCCAGCGACAGAGCCATNCAAAGAAGATGCTTGCCTTCAGAAACTCANGATAGAAAAC
ATTGAATCGCGAAANGGTTCCGAAAAAAAGANAATAAACTTTTGNCATTNAANACGAGCAGGAGGGT
CGGTACCGGAGTTGAAAGCACCCCTTCGAGCCAGCAAAANCCCCTTCANGGCCNCCCAGGTGGGTTCG
GACACCAAGAAAACGGGNGTCTGGTCCGGGGCACTTGGTCGNNTTTTACCGTNGGAANCCGTNGNAA
AAAAGGTCCTTTCGGGAACCNAAGGTTTGTAAGNCCCTGGGCCNCCCAAGGTNNTTTTAAAGGGGAA
AACCCGNGTTTTTNCCANCCTTTCGTTTTNCCATTNGNCCCGGNNTTAANTTGGCAATNAAAAATTNTT
NAAACTTCNAAAAAAAAAT
TTNAACAAANCTGGAGCTCCCCGCGGTGGCGGCCGCTCTAGAAGTAGTGGATCCCCCGGGCTGCAGGN
NNNNANGGNGGNGNTCNTNGANCTCGCNCTGCTGGAGNGGGGNCNNGGGCNGNNNNNNGGNNTCNA
GTGCNGCGCTNAAGNCGAGCGCCAGTTCCAGTTCTTCAGACAAACAATGGTCCGAGTCTAACGGGAC
TGACTACCATAGCAGCTCACCTAGTCAAGCAGGCCAACAAGGAGTATTTGCTGGGGAGCACGGCAGA
GGAGAAAGCCGTGGTTCAGCAGTGGTTAGAGTACAGGGTAACGCGAGTGGACGGACACTCCAGTAAA
GATGAGATGCACACGGTCCTGAAGGATCTTAATTGATATCTTGAAGATAAAGTCTACCTTACAGGATA
TAACTTCACATTAGCAGATATCCTATTATATTATGGGCTTCATCGCTTTATTGTTGACCTGACAGTTCAA
GAGAAGGAGAAATATCTTAACGTGTCGCGCTGGTTTTGTCACATTCAGCACTGTCCAGGGATTAGGCA
GCATCTGTCTAGTGTTGTCTTCATCAAGAACAGACTATATACTAATTCCCAGTAGAAGCCACCCGCGCC
CCGGCCAGAAGAAGGAATAACCCATGTCCAGTGGGNAAGTGNGGTTTCNGANCCANAGGACTGACGT
GNACCTGATTTCNTTCATTTCTTNTATTTGGTNGAAANTTGGTAAAAATTTAAAGTACCAACCTCTTGT
CCCCAAGGATTTTTGGGCTGGGTTTTTTAAANTGGACCANTTTTTGCCAAGTTTTTTTTTTTAATGGNAA
AAAACTTTTCAACCTGGGTGGGCCAAAAACCTTTGGGGTTGGGAAAATCCAAGGTTTTTTCAAGTTNTT
TNTTNGGGTTTTTTTNTTGGACCCTTAAGATTGGAAAATTTTTNTGGGTNAAANAAAAAAGTTGGCCA
GCCNATNNGGAGNNCGCATNACAGGGGGCGGCCGCTCTANAACTTGTGGATCCCCCGGGCTGCAGGC
TCAGGGGAAAAANGNNNAGTNCTTCTAGAAAGTACNAGNGCTTGTGCCTGCTCCTGGGGGGAAGGG
ACCTTNNCGGATNTGAGGGTTAANCCTTANAGAAATNAGCTGTTGTGTAATACAANGANATCTACAAT
GACACCGACAGCCTGTTCCCAACAAGTCCTNNGCATCCTCTTCCTAAAGAGGACCCTTTTCATCTCTCC
TAGNAGACCAGGAAGNATNTTCATCCTACCCAGGAGAAANTTGTAGGAGAAGGCGCCTACAGAGGAT
GACTTCTAAGCGATGTTTGCTAAGGAGAGAGGGCACNGANAGATGCCATAATACNTTGATTGGNCTGT
GAATTTAACCTCCCTACCGTGGTAACCTCACNCNCTGTGCCANNGGCCTTGAGTTGTGTGGAAACAGC
CCANAAGGCATTAGNCTACNTGCAGGCTCTTGGGTGCTAAANAGCTTGACCTTCCTGGGCANNGTNTG
CCTGNATTGGNCCNGTCGANCGNNANNTGGAGTTATAANGATGTNTGGCTTCCCATTGGTNCCACTTC
CAAGTCCTGTTTCACACNCCATANATACNTTTAAGCCNNGTGCANNANACNTNCANAANTNTGNGNAT
TTCGCTGCGGNGCTATCGANTGAAANATTNTATGNNGTCCCNGNNTCTNANNANNGCGNCCNTTGCTG
GNCCNTTTGNTNGNANGNGGNCCTTCTATCTAAACCTGTAANNGGNAGTCACNCTANNCNNTCTCTTC
ATNGTCCTTCCNANTGGNGNGNNNCCTTNGGAACCTCTCCTTTNGAGNNCNNNNNNANCGTNCCNGGT
CNAAAATNANGCCTCGGAGNTAATNTCNTTTANTNTTNNNANCAACTTTTTTGNNNNNNTAATATTNG
NATCTTTNTGGNNNTTCTTNTCNTNCTGNNNTNCCTANTNTNTNANCNNTATTCNGNTTNCNCNNNGA
ATTGCNCNTCNNCANCTTNATCTCNTATATNCCNCTNTTCNTCGNAANNCNNCCCTATTAATCTTTNNT
NACCC
NCAANANCTGGAGCTCCACCGCGGGGGGCGGCCCGCTCTAGAACTAGTGGAATCCCCCGGGCTTNAA
GCCGNTNGCGGAANCCGGTGGGGAGGCGGCTGTCGGGGCTGAGGGCCGCCACAGGGCGCCGGCGAGA
TGGAGGTGACGGGGTTGTCGGCNCCCACAGNGAACGTGTTCATCAGCAGCTNTNTCAACAGNTTTCGT
TCCGAAAANCGGNNCAGNCNTAANCCTNCCGNACNTAATTNAAANGNNAANNANCANTTNGTGTGGG
GCAACCTGNTTCTTGNNTGTNANANNAGCTGTNTGNACCTGACAACANNNTTTGTTGTAANCTNNATC
ACNATGATACGCTNCTGGGCTCCTACCACCCAGANGACGACTGGTGCATCCNTNTTNTTGNCCACCAC
GGNGCCCCCCTTTNTGAGNATGAATGACATATCTAAGNNGGGNNAATATGNAATATCTCNNAAAAGC
NNTTTGTANAGAAAGNGAGGAANTANTCNNTCCTTTCCTTAAANGCANCANAANNNGGNTNNGTNTAT
TTAAAAAAAAANGGGGCCNCCCCAANGANNNGGAAANATCACNTCCCTTTTTTTANGGAGGAGGACG
GGNCAAGCCATANTNNTTGTGTGCAGCCCNTTNGGATGNTANGACCCCTGAGCCAGCCCCCNTCCCAT
NGGGCNNCCGTACTTNTTTTGTNNNNTTGNACNNANTTNAAACCNNGNNTTTTGGTATTNGGGATTCG
NNTTTCNNTTAACCCCAAACGNAAAAAANCAAAANNCNCTNGGCAATGGTGAAAACTTTTTTTTTATT
TTTGATNGCNANGNNNNGCTNTNNTNNATTCTTTTTTNNNTTAAAANGGGGNAAANTTNTNGCAATAA
AAAANAANAGNNNTTTNGNANTTAGANTGNNACCNNAANGGGNGAGGGNNNNNNTCTTTNNTTNNN
CTNNNNCCCANATTNCCTTCTTCTTTNNTNNTNNCCNGGGGTNCCCCTTNNNCCNTTNCCNNTTTTNN
NTTTTTTTACNTNCCNNCCCCNCCN
GCCCTATNNGGAGCTGCANCNCNGGNGGCGGCCGCTCTAGAACTAGTGGATCCCCCGGGCTGCAGGG
NACNTTGNGTANAGAGATCCTCTTGCGAGAGCTANAGGTGCCGGCGNGCNTGNCCCTGGAGANCNTT
CTGATTCCCTTCCTTNNGGNNATGGGGGATCANAAGCNGNTTTNANACCTTTTTAAGGTNCCGGNTAG
NACAAANGANCTNGNGCCNGCCCATTNTNNACANCCNTTGNACCTTCNGGAGCCCGAGAAACGAAAA
GAATTTNTTAACANCNNNCTTGGCTTGGCCTTTTNAATNCNAAGANTNNNGGNACNTNCGAAANCNTT
ACNCNCTTCNGGAACNGCNGANCNTTATNGANGNAAACGATCGGNATNTAGAANTAANGGGNCAGA
NTGNNGTNTTCCNGAAGCACCCCCGCAAAGCGGCCATTCTNAACATGCCGNTGGNGACNACNCTCTTN
TACTCNNGCTTNTACCACTACACACGAGGCTGAGGGGACGTTCNGCNGCCCANNGCAACGTGTNAGAN
AGACGTGTANGATNCCACACAAACAGGTACANGCTGNCACCGCTGTCTCGCTCNCGCCCCNNCTCTCN
GGCCCTGNGATTGNCATCAGATGCCCCTGTNTACCACCAACNAANATGTNTGGTGCTNCCCCCCACNA
AANANAGCNGCCCGCNCTGANNNCCCCTCNGNNNCGTGGNAGATTTTTGCNCCANCNAATATTGGCCC
CCNGTTNCAGGANNTTTCNNGANGNGTGTNCCATCNTNGTGNAAGAATGCCCTCCCCCACNGNNGGA
ACTTNNGCCCCCNCCNGGTCCCACTAGTNNCTTGATTGNNGGGNNTTGGTATANCTTTNCCGGNCGAN
CCTNNAANNNATGNNTCCNTCCTNTTTNGTTTCNCACNNCCCNGCANNCNNCNNGNGGGCTNTANANG
GNTTCTTTNNCGGNGGAAGGAAGAAAAAANTNGANTNGCCCNTTCTTNAANANANC
NNCAAANCTGGAGCTCCACCGCGGGGGGCGGCCCGCTCTAGAACTAGTGGATCCCCCGGGCTGCAGG
ATTNCNNCACGAGGCCNCAGTGTGGNACNTNTGGNNTNNACNNTCGANCAAAAGCNNCGTNGCCTGG
TNNNCGGANAANATTNGNGGANTTNNAGNCCCCAAGGGGGNCTTACCNTTCCNGGGCCGNCCANCNT
TTTNAGGGNTNNNACCCGNNTNGNGNTTCCCTTNCNNACNATAGTTNCNTTATTTNNCCANNACNGAA
ANAAGACNCCGGAATNANAATGAACNTGGGCCCANNNNTGGNTCGNCGGACCNNTNCTTNGAAGTAA
GGTTCTGGNCCCGGGGGTNGCTGGGTTTNCATAAACGGGGCGTCTNCATTGGGGGCTCTATGCCGCGA
NCCATNTTGTGGNCATTTGAGTNCANNGGATAAANTCTCTCTNTCTATNCACGNGGGGGGGGGGGCTG
TCTCTGAGAAANNCACTCCTGTGNCCCNNTTTCTCTNAANAAATGGGGTGGGGCANAATCTCGTGNTT
TTTTGCNGCCCCTCTCCNGTGTGANANGGCNNGATGNCGCTGNTTCTTACTANTAATAATGCNCTTNAT
ACCCNNNATAAAAANNTTTTAAANCANNNGGGGGGGGCCNNNCCCCCNNGGGGGGCNCNNGGGGNN
NNNTTTNTTNNNNNNNNNNNNNNTNAANNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNN
NNCAAANCTGGAGCTCCACCGCGGGGGGCGGCCCGCTCTAGAACTAGTGGATCCCCCGGGCTGGAGG
ATTNCNNCACGAGGCCNCAGTGTGGNACNTNTGGNNTNNACNNTCGANCAAAAGCNNCGTNGCCTGG
TNNNCGGANAANATTNCNGGANTTNNAGNCCCCAAGGGGGNCTTACCNTTCCNGGGCCCNCCANCNT
TTTNAGGGNTNNNACCCCNNTNGNCNTTCCCTTNCNNACNATAGTTNCNTTATTTNNCCANNACNGAA
ANAAGACNCCGGAATNANAATGAACNTGGGCCCANNNNTGGNTCGNCGGACCNNTNCTTNGAAGTAA
GGTTCTGGNCCCGGGGGTNGCTGGGTTTNCATAAACGGGGCCTCTNCATTGGGGGCTCTATCCCGCGA
NCCATNTGTGGNCATTTGAGTNCANNGGATAAANTCTCTCTNTCTATNCAGGNGGGGGGGGGGGCTC
TCTCTGAGAAANNCACTCCTGTGNCCCNNTTTCTCTNAANAAATGGGGTGGGGCANAATCTCGTGNTT
TTTTGCNGCCCCTCTCCNGTGTGANANGGCNNCATGNCGCTGNTTCTTACTANTAATAATGCNCTTNAT
ACCCNNNATAAAAANNTTTTAAANCANNNNGGGGGGGGCCNNNCCCCCNNGGGGGGCNCNNGGGGNN
NNNTTTNTTNNNNNNNNNNNNNNTNAANNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNN
TTNANAAAATCTGGAGCTCCACCGCGGGTGGCGGCCGCTCTAGAACTAGTGGATCCCCCGGGCTGGAG
GGCCGGGTTGAGAGGAGCGTGGCCTTCTCCTCTCCCCGCCATGGCGTGTGCTCGTCCACTGATATCAG
NGTACTCCGAAAAGGGGGAGTCCTCTGGCAAAAANGTCACTTTGGCTGCTGTGTTCAAGGCTCCCATT
CGACCCGATATTGTTAANTTTGTTCACNCCANCTTGCGCAAAANCANCANACAGCCCTATGCTGTNAG
TGAATTANCAGGNNATCAANCCAGNGCTGAGTCTTGGGGTNCCGGCANAGCTGNGGCTCGAATTCCC
AGGGTTCGAGGTGGCGGANCTCACCGTTCCGGNCAGGGCNCTTTTGAAACCNTGTGTCNNGGGGCCCG
ANNGTTTGCCCCANNTAAAACNTGNCAACGTTGGCCCCNGNAAAGNGAATNCAACCCAAAACCNATN
CCCNTTNNGTTTTGCANTGNTTGCCTAAACCTTACNAGGGNTGGTNATGTTTAAAGGNNATCNTATAA
AGGAAGTTCCTGAACTTCCTTTGGGGGGGGAAAANAAAGTNGAAGGTTACAAAAAAACCAAGGGAGG
TTGGTTTNNTTTTNAAAAAACTTAAGGCCTGGAATGAATTTCAAAAAGGGTNTNTNCCNTTTAAGNAA
TGAAACCTGGCNAAGGCAAAAAANAAAAACCGGNNCCCGTNTCCAGCCCANGGGGACCCTTGGTTTT
TTTTTAAGNGGGGCGAANGGNTTTTTTCAAGGCCNTTNAAAAAAATCCCCGGGAAANACCCTTGGNTN
AANGGGAACCCAACCTTNAACANTTTTGNAAACTTTTTCCCCTGGGGGGGNACCNNGGGAAACNTTTT
TTGCATTTTGGAACTGNAAAAGGCTTTTTCCCAAAAGTTTNAAAAAAANCTTNTTTGGGNCCNTTGGG
GGAAAAAACCNCCCNCCN
NAAAAGCTGGAGCTCCCCGCGGTGGCGGCCGCTCTAGAACTAGTGGATCCCCCGGGCTGCAGGAATTC
GATATCAAGCTNATNGATACCGNCTACCTNGNGGGNNGGCCNGGGNCCCANTTTACCCTATAGTGAGT
CTGATTANAGAGGNCCCTCCCAAAACCCTTGTTGAAACAGCCAANGAAGCCAAGGAGAANGCCNANG
AGACGGCACTGGCAGCTACAGAGAANGCCAAGGACCTTGCCAGCAAGGCANCCACCAANAAGCAGC
AGCAGCAGCAGCAGTTCGTGTAGCCAGCCCAGGCCCTGAAGGANCAGCCTAACANACTACCANCTCC
AGGCCCTGTGCCCCCTCCTCTTGTACTTATTATTAAAAGCCAGCTTCTAGGCGTCTCGAGTGTCTGGGT
GGTGGGCTGAGGGTAGGTGCCCTGTTTGCATCTCCAGCATAGCCAGCTGGTTACCCATATCTGAGCCG
GCTTCCTTACCCCTGCATCGGGTGGCTGANGACAGAAGCACAGTGACTTGCCCACGGTCACCGNATCC
ANGTGAAGTGGTCATGAATGCTANACTGTAAGCTCCATGAAGGCANGGACCGNTGTTTATCCTTCTGA
TGTGTCCCCAAGTATNTTGAAACAAAATTTGCGTGTAACANGCACTTCAATAAATACTTTGTTGAATTC
AATCGGACCCCAGAANAAAAAAAAAANNNTNTNNNAANTNTNGNGGGGGGGGGCCCGNCCCCAANT
TNNCCCTNNTGGGGGGGGNNNNTTAAAANNNNNNNNTTNNNTTNNNNNNGNNNGNNNNNNAAAA
NNNANTNNANNNCNCCCNNTNNNNNNGNNTNTTAANANNNNNNTTTNAANNNNNNTTTTNNNNNNTT
NNNNNAAAANNNNNNNNNCCCNNNNNNNNNNNNAAANNNNNNNNNNNNTNNNNNNNNNNNNNNNN
NTNCN
TNNNNNAAAAGCTGGAGCTCCACCGCGGGTGGCGGCCGCTCTAGAACTAGTGGATCGCCCGGGCTGC
AGGAATTCGGCACGAGGGCTCAGCCTGTCCATCGAGGGTCCCAGCAAAGTAGACATCAACACAGAGG
ACCTGGAGGATGGCACATGCAGGGTCAGCTACTGCCCCACGGAGCCTGGCAACTATATCATCAACATC
AAGTTTGCTGACCAGCACGTGCCTGGCAGCCCCTTCTCCGTGAAGGTGACAGGCGAGGGGCGTGTGAA
AGAGAGCATCACGCGCAGGCGACGGGCCCCATCGGTGGCTAATGTTGGCAGTCACTGTGACCTCAGCC
TCAAGATCCCAGAAATTAGCATCCAGGACATGACAGCCCAGGTGACTAGCCCATCAGGGAAGAGCCA
CGAGGCCGAGATTGTGGAAGGGGAGAACCATACCTATTGCATCCGCTTTGTGCCAGCTGAGATGGGCA
TGCACAGGGTCAGCGTGAAGTACAAGGGCCAGCACGTGCCCGGGAGCCCTTTCCAGTTCACCGTGGGG
CCCCTGGGGGAAGGGGGAGCCCACAAGGTCCGTGCCGGGGGCCCTGGCCTGGAAAGGGGTGAAGCTG
GAGTGCCAACCGAATTCAACATTTGGACCCNGGAAGCTTGCCNCGGGGGGCCTGGCCATTGTTGTCGA
GGGCCCCAACCAAGGGCGAAAATTTCCTTCAAGGACCGGAAGGATGGGTTCCTTGNGGCGTGGGNTTT
ACGNGGNCCANGGAGCCAGGNGAACTTTTGAGGGGNCNNTCAAGTTTNAANNGGGGGGGCCCATNCC
CCCCNAAANNNCCCNTTTTCGGGGGGCCCNGGGGGGTTTTTCCCGGTGGGGGACNCCCCCCCCCCCTT
ACTGGGTTTTAAGGNTTTTAGGGAGNNANGGGGNTTAAAGGGGNAANCCCCCCCCCCCCCNTTTTTTT
GAANCCANCCCCGNAATNGGGGGCCCAANG
TNNNNAAAACTGGAGCTCCCGGCGGTGGCGGCCGCTCTAGAACTAGTGGATCCCCCGGGCTGCAGGA
ATTCGGCACGAGGGGNGTTGCTGCTNCTTGCGCTGTGCGCCGCANGCGCCCGNNGGCTTTATTNCCAC
NTAGGANANACCGAANANCGCTGNTTCATCGAAGAAATCCCCGACNAGACCATGGTCATCGGGAAGT
ACCGNACNCANNTGTGGGANNANCAGANGGAGGNNTTNCTGCCNTCGACCCNCGNNCTGNNCATGCA
CGNGNAGGTGAANGACNNTGAAGGCAANGTGGTGCTNTCNCGGCAGNACGGATCAGAGGGCCGTTTN
ACCTTNNCTTCCCACACTCCTGGAGACCATCAGATTNGCCTGCACTCCAACTCTACCAGGATGGCTNTC
TTTGCTGGCAGGCAGACTGCTTGTACACCTAGACATCCAGGTTGGGGAGCATACCAACAACTACCCTG
AGATTGCTNGCCAAGGATAAGACTTGACGNGAGCTAGAGCTCCGAGCCCGCCAGCTGCTTGATCAGGT
GGACANATCCAGCAAGGAGCTTGGATTACCAAAGGTATCGTNAAGAGCGCTTCCGTCTGATNAGTGG
ACANNACCACCCANAGGGTTCTGNGGTGGNCCATNGCTCAAACTGTTATCCTTATCCTTCACTGGNAT
CATGGCCNATCCCNANCTTAAGAACTTCTTTTTAAGNCCAAAAGCTGGTGNAAATGGCCTCACCAACG
NGAAACCTTTCCTGGGANCCTAAAGNAATTNGGTACCTTTCCCCCACNCAATATCNTTATCCCNCCTGG
AATTTTTGAGGGAAAAAAAAAANTGAAAAAAAANAATNTTTTCCCCCNTTTGGTTNCCTTNGGNNNCC
AANGNATTTNNNAATNCNNCCCCNTTTTTNAAAACTTGGT
AAAACTGGAGCTCCACCGCGGTGGCGGCCGCTCTAGAACTAGTGGATCCCCCGGGCTGCAGGCTTTGC
TCCAGTCAATGTAACAACTGAAGTGAAGTCTGTAGAAATGCACCATGAAGCATTGAGTGAAGCCCTTC
CTGGGGACAATGTGGGCTTTAATGTCAAAAACGTGTCTGTCAAAGATGTCGGTCGTGGCAATGTGGCT
GGTGACAGCAAAAATGATCCACCCATGGAAGCTGCTGGCTTCACAGCTCAGGTGATTATTTTGAACCA
TCCAGGCCAAATCAGTGGTGGATATGCACCTGTGCTGGATTGTCACACAGCTCACATTGCTTGCAAGTT
TGCTGAGCTGAAGGAGAAGATTGATCGTCGTTCTGGGAAAAAGCTGGAAGATGGCCCTAAATTCTTGA
AATCTGGTGACGCTGCCATCGTTGATATGGTTCCTGGCAAGCCCATGTGTGTCGAGAGCTTCTCTGATT
ATCCTCCCCTGGGCCGTTTTGCTGTGCGTGACATGAGACAGACAGTCGCTGTGGGTGTCATCAAAGCA
GTGGACAAGAAGGCAGCTGGAGCTGGCAAGGTCACCAAGTCTGCCCAGAAAGCTCAGAAGGCTAAAT
GAATATTATCCCCAATACCTGCCACCCCAGTCTTAATCAGTGGTGGAAAGAAGGGTGTCAAAACTGTT
TGTCTCAATTGGCCATTTAAGTTTAATAAGTAAAAGACTGGTTAATGATAACAATGCATCGTAAAACC
TTCAANAAGGAAANGGAGAATGGTTTTTGTGGGACCATATGTTTTGTGGGGTGGCAANTTTAAAGTTA
TTAGTTTTTTNAAAATCAAGTACCTTTTTTAATGAAAAACCAAGCTGGCCCAAAAAATCTGGNCACAG
AAATTTGGAGACCCNTTTAAAAAAAGGTTTTAATGANNAAAAAAAAAAAAAAAAAACTTCGNGGGGGG
GCCCGGNCCCNATTTGCCCTTTNNTGGNGNNNNNNNNNTTNTNANNNC
CAAANCTGGACTCCACCGCGGTGGCGGCCGCTCTAGAACTAGTGGATCCCCCGGGCTGCAGGTGCTAG
TTTGAAAGTGGCTGCAGCCATTGCTGGCTATGTGTTTAGAGACAAGGTGAGATCAGAATTTAATAAGG
ACTTCCGGCAGCAGATGAAGAATTATCCAAAAGACAACCAGACGGCATCGATCCTGGATAAGATGCA
GAAAGATTTTGAGTGCTGCGGGGCGGCTAACTACACAGACTGGGAGAAGATCCTGGCCGTGACCAAT
AAAGTCCCTGACTCCTGCTGCGTCAATATCACTCATAACTGTGGGATTAATTTCGTTGTGAAGGACATC
CATACTGAGGGCTGTGTGGAGAAGATTGCGGCCTGGCTGAGGAAGAATGTGCTGGTGGTGGCTGCGG
CAGCCCTAGGCATTGCCTTTGTGGAGATCCTGGGTATTGTCTTAGCATGCTGCCTTGTGAAGAGCATCC
GAAGTGGCTATGAGGTGATGTAGGGGGTCTGTCTCTTCAGCCCCCTCATCCAGGGGAGTGGGGTAATA
TACTCCAGGTTTTTCAATTAAACGGATTATTTTTTCAGACGCCAAAAAAAAAAAAAAAAAAAAACCTG
GGGGGGGGGNCCCGNCCCCNNTTCNCCNTTTANGGGNGGNTTNANNGNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNCNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNC
NAAAATCTGGAGCTCCACCGCGGTGGGGGCCGCTGTAGAACTAGTGGATCCCCCGGGCTGCAGGGAAT
TCGGCACGAGGTAAACGAATACATTAACGGTGCTAAACACAGAACATTTTAAACCCCAGGTCCCGGAC
ATTTGTAAGCTGGTAACCTTCAAGCCTTCTTCACGGGCCTTCTTGNTTACCCNTGAATGGGCCCGNTT
TNCTTTTGNCGGGGGNNCCTTCNNCCATTGGCTTGGNGGNACATGGGNGGAACANTTGATCTGNTNNA
NNCTGGCTTNAANACNCNNTGGNTTTTTANAATTTNTNNNTTNTGGTNAAAANNNATNNTNNCCNAN
NNAAANNTTNGGNTAANTTTANNTTNAGGNATTGGCNNGCNNGGGGGCATNCCTTANNGGNCNGNAN
ANNGNCNAAGGGAAATAACNNGCACCTNANGGTGGCNANGATNCNTCTAAGGCNAAANGAACAATG
GTGGGAGANACCNGCCNAATCCACCACNCCCNAACTGGAGATAACNAATCTTATGGTCACNGGGCNG
TGACNGACACAAANGAGAACTGAGAGGTGTGGGTCTGANAAGNNCANATAACTCTTCTACACTATTA
CNCCANATTCTTTTCACATATCCCCCTCTCTCACTTTTGCNGAAGCGCANTTTNTTTTTTTGCNCTNTN
TNNCNAATNTTGCTCNNAACNNTTTTCAANAGACANNTNNGCCACNNCCCNTCTCTGGNNATCCCCCC
NCACCGTCTGTNGAGNGGGNNTAACCATGTGCTGCGGGGGTGGTTCCCCTCCTAAANTACTCTTTTTTG
AAGATGCAGCGACANNGGNGGCTTGTTTTTNCCCCCAAAANAACCNCANTTCTNNAAGCCGAGGGGC
CCCAACGTNGGGGNANAAAAACAAAAAAANGGCGGGGGGGGNGGAAATGGGGGTTNCTNTTTGGGG
GGGTTCCNGGGGNCCTTTTTTTCAACANCNTGGANGGNGNGNNCNCCACCNN
AAAACTGGAGCTCCACCGCGGGGGCGGCCGCTCTAGAACTAGTGGATCCCCCGGGCTGCAGGANTNN
NNCACGNGGCCNCAGTGTGGNAGCTATGGNATNTACNNTCGANTCAAGAANNACGTNGACTGTANAN
NGAANACAATGCATGAGTATNNNGCNCCNAGGNTNCACTAACCATNCANGGNCCACCANNCTCTCTA
AGGGCTGNNACCCNTNTNGACTTTCTCTTNCTNACAATAGTTCCATTATTTCACCATGACTGAGGACAG
GACACGGNAGTGANANTGAGCTAGNGCCANGACTTGNTTGNCNGGACACTGNNTNGGAGGTACGNTN
CTGNTNCTGTGGCTGNNNNNGTNNTNCANNANNNGGGGNCTANCTACATGGGGGCCTCTACCNCGAN
NCCNTNCTNNGGACTTGANNGTACANGGNANACCNTCTCTTTNTCTATTCATGGGGTGGGGGGTCCTC
TNTCTGANATAACCCATTCCTGTCACCANTTATTGACTGATNAAANTNGCTGNGANGGCATTCTCTTGA
TGCCTTGNNGGCATCCCCCTNNACCTGTNTNAANNGGACCACATGGCCNTTTGNTATGNACTCGGAAT
NAAGCNTGNNTATAACCTTTAATAAAATCTTTTTAAAACACGGGGGGGGGGCCCNGCCCCCNTGGGGG
CCCNNTGGGGGCCCCNATNNCCCCTATGGGGGNGNNGNNNANCANNNNNNCNNNNNNNNNNNNNNC
NNTNCNNNNNNCNNNNNGNNCNNNCNNNNNNNNNNNNCNNNNNNNCNNNNNNNNCNNNNNNNCNC
CCNNNNTNCNNNTNNNNCCNNNNNNNTNNNNNNNNNCNNNNNNNNNNNNNTNNANTNNTNNNTNNG
NNNTNTCNCNNNNNCNNCNNNNNNCCCNNNNNCCNCTANCNNNNTNNNNNCNACNTNNNNNNNNCN
NNNNNTNCCC
AAAACTGGAGCTCCACCGCGGGGGCGGCCGCTCTAGAACTAGTGGATCCCCCGGGCTGCAGGANTNN
NNCACGNGGCCNCAGTGTGGNACCTATGGNATNTACNNTCGANTCAAGAANNACGTNGACTGTANAN
NGAANACAATGCATGAGTATNNNGCNCCNAGGNTNCACTAACCATNCANGGNCCACCANNCTCTCTA
AGGGCTGNNACCCNTNTNGACTTTCTCTTNCTNACAATAGTTCCATTATTTCACCATGACTGAGGACAG
GACACGGNAGTGANANTGAGCTAGNGCCANGACTTGNTTGNCNGGACACTGNNTNGGAGGTACGNTN
CTGNTNCTGTGGCTGNNNNNGTNNTNCANNANNNGGGGNCTANCTACATGGGGGCCTCTACCNCGAN
NCCNTNCTNNGGACTTGANNGTACANGGNANACCNTCTCTTTNTCTATTCATGGGGTGGGGGGTCCTC
TNTCTGANATAACCCATTCCTGTCACCANTTATTGACTGATNAAANTNGCTGNGANGGCATTCTCTTGA
TGCCTTGNNGGCATCCCCCTNNACCTGTNTNAANNGGACCAGATGGCCNTTTGNTATGNACTCGGAAT
NAAGCNTGNNTATAACCTTTAATAAAATCTTTTTAAAACACGGGGGGGGGGCCCNGCCCCCNTGGGGG
CCCNNTGGGGGCCCCNATNNCCCCTATGGGGGNGNNGNNNANCANNNNNNCNNNNNNNNNNNNNNC
NNTNCNNNNNNCNNNNNGNNCNNNCNNNNNNNNNNNNCNNNNNNNCNNNNNNNNCNNNNNNNCNC
CCNNTNCNNNTNNNNCCNNNNNNNTNNNNNNNNNCNNNNNNNNNNNNNTNNANTNNTNNNTNNG
NNNTNTCNCNNNNNCNNCNNNNNNCCCNNNNNCCNCTANCNNNNTNNNNNCNACNTNNNNNNNNCN
NNNNNTNCCC
TTNNNNAAANNCTGGAGCTCCACCGCGGTGGCGGCCGCTCTAGAACTAGTGGATCCCCCGGGCTGGAG
GAATTCGGCACGAGGCCTGAAGACTCTGGTGGCTAAGGGGCTTCATCCCAGCTGAGAAGCGAAGAGC
CGCAGGCGGCCGCCGACTTCACAGCCGCTCGGGGTTTTTAGAGCTTCAGCTATAAAAATGGGCAGGAT
TTTCCTTGATCATATCGGTGGTNCCCGTCTGTTTTCCTGTGCAAACTGTGATNCGANCNTGNCCANCCG
TTCAAAANTCATCTNTACTCGGTTCACAGGCGCCANTGGCANAGCNTTTNTTTTTANCAAGGNAGTTA
NCCTNCNGTNTAGNGAAGTTTNAGAATCGGGNNNTNCTCANTGGCCGCCACNTGGTTCNNNANGTGAG
CTGGAAAACNNGCATTANCAANCTGGGATGGATNTATAAGTTTGGCANTGANAACNGCCAGNGTTAT
AAGGAAGGNCGNGNNATCNTGGACCGCCCCTTTTANTNCANAAAAAGNNAGGGCTTTTGAGGGANCA
TGTNCNNTCTGATACCTNTGGAAAAAAAAAAANAAAAATCCNTNTTTTCCCAGGTCTCCTTTCACTGA
AAAACAAAAANTTTACTTACATACCCTTGTCCCCTTAACATTNAAAGTCGGATTTCNTGAACCTGGGG
NAACAAAAANTTTGGGGAAAAATTTTTGAAGGGANCCTTCCCTTTTNTTTCCCTTTTTTTTTTTTNAAA
ATTNNNATTTTTCCCCCCCCCAACACCGGGGGGTGGAAAANAAAAATTTTTCCCCAAAGNCCCCGTTT
TTTTTTTTTTTTTTTCGCCCCGGNGGGTNCACCCNNNGGGGGGGGNAAAAAANTTTTTTTTCCCCCCCC
CNCCTGGGGGGGGNTTTTGGGNGAGNAAAAAAAAATTNGGGGGTTTTTTTAAAAGGGGGGGAAANNT
TTTTTTTTNNAAAATTTNNNANAGAGGAAAGGGGGNNCNAAAN
AAAAGCTGGAGCTCCCCGCGGTGGCGGCCGCTCTAGAACTAGTGGATCCCCCGGGCTGCAGGAATTCG
GCACGAGGCNAGNCTNTTTTNCCNNTNNCNTNGGNNGNCTGAANGCTTGGANCCCCTCCCNTNGGAA
NAAGANGCNTAAAACTCNGANANCGNCGGNTACATGCCATTNNTGAGGNCACACCNCCGAAANNAA
NTGGGTCCAACCANCTCCAAGCTTGCCNAATTCGGNGNCACGNAACATGNTAGGAANGCCTTTTTTGC
CNNACCCGNCGATGNAAANCGAGCNTCACNTTTGTGGNAGANCCNANTTCANANAAAANAAGGAGG
GNTTTCAACNACAGATTTTATTAGNATACNACATNGATACTATANNTCNCCCATGGNACAAATCCATG
ATTNAGAGGATNNTAAGANCGAGGCANNCAGGGNGNTTTATTTACANGAAACATTNNNGCCTTTACC
GAATNNATCTCCNGATGCNTNNCTTTTCAATGCTGTGCACTCGNGGAACAAAAATAAAATCCCCTGAT
TGCCAGGNATTGACCCNATNAATGGGAACGCACTTTNTNTNCTNACCCACAGACCAATTNTTAAGTTC
CNCCNCCTTTTTATGTGTTGATTTGCCCAAGNCCGCCTTATGANCTTTTTCTTGANACAAATNGNAANC
GGGACCTAACATAACCNCNCCTTGNTTCCCCGGGACCCTTNCAAAAATTTAAGCCTTGAACCGTTTTNC
TCGGCNACCCGGNGGGGCTTGCCCTANCCCCCCNCTNGAAAAAAAANGGGGAATTTTTTAAAAAAATT
TTTNTCCNAAAGNGGGGGGCCCTTTAAACCCCAGGTNGTTNTTAAAAACCCNCCAANTTTCCTGNAAA
AGNGGAAAGGGTTNCCNTTNGGCCTTAAAAAAANCCCNTTTTAAAAAATTTTNTTNGGGAAAAAAAG
GAAATTGTT
NAAAAGCTGGAGCTCCACCGCGGTGGCGGCCGCTCTAGAACTAGTGGATCCCCCGGGCTGCAGGCAA
CAACCTCTTGAAGTGACCGTTCCTGCCCCAGGCTTCCAACACAGCCATGTCTCTGCTCCTGCTGGTGGT
CTCTGCCCTCCACATCCTCATTCTCATCCTGCTTTTCGTGGCCACTTTGGACAAGTCCTGGTGGACTCTC
CCCGGCAAGGAGTCCCTGAATCTCTGGTACGACTGCACATGGAATAGTGACAACAAAACGTGGGCCTG
CAGTAACGTCAGTGAGAACGGCTGGCTGAAGGCAGTACAAGTCCTCATGGTGCTTTCCCTCATCCTCT
GCTGTCTGTCCTTCATCCTGTTCATGTTCCAGCTCTACACCATGCGGCGAGGAGGGCTATTCTATGCGA
CTGGCTTCTGCCAGCTTTGCACCAGCGTGGCGGTGTTTACCGGGGCGCTGATCTACGCCATTCATGCCG
AANAGATTCTGGCGGACCGCCCGTCGGGGGGCAGCTTTGGTTACTGCTTCGCCCTGGCCTGGGTGGCC
TTTCCCCTCGCCCTGGCCAGCGGCATCATCTACATNCACTTGGGGAANCCGGAGTGAAGGCTTGCNCC
TNCCCTTGGTTNCCCAANCGANTTCCCGGGCCCTTCATTNTAACCCGCAAGCCCCCANCCTTTCTTCNT
TTTTATTTTAAACAAAAAAATAAAACTGGTTAANCCGCCGGAAAAAAAAAAAAAAAAAAACTTGNNG
GGGGGGGCCCGGANCCCAAATTNNCCCTTTANNNNNGGGGGGNNNNNNNNNNAAAANNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNN
TTTGANNCNATCTGGAGCTCCANCNCNGTGGCGGCCGCTCTAGAACTAGTGGATCCCCCGGGCTGCAG
GAATTNNNCACGAGGATNNTNNNTNNCTGANNNANTGTTCAAGNACNGCNCTGGGNANNNTTANTNN
TGGNGNGTCNTNCNCTGNNGANNCTTANTATNAAANTGNNTNTNNNNNTCACTTANNACAAGATCTG
NTGCANNCNCNCAGTGNTNNANNCCNTCNNACNGNGNTNCTANTGNCGATGGAATNNNGNTATGGTG
ACGNNGACCNANACNACCNTTNNTGANATNCTNTGTNNTANANNNCCNGATTTGNTTNNGANAGTGA
NNCATCATANTCTTNGGGNTGCCCNTGATCNTNATCANGNTGNAGTATNTANACCNTGCACTGANGNG
GGCTATACATATANGACGTGTCTTTTGCCATGTTTGCNCCACNCANGGTATAGTGTTANTNGGATGTAA
TNATCTAAGCCGANTTNTGCNGAAGACTGTCTTGNATTANCCCTNNTCTTNTTGNAAGAATTTGNNTA
NCTGATTCGANCTTGGGNTAGTTGGGCNTTTTTCCCTTTNGCNTTGGGGAAGCTTTATNAATNATANGT
NCCACTTTGNGACTATCTGNCAGNTTTANGGNCTTGTTCCNTTGCCAATTGTTTTAGCNNTTTNCGNCC
CNCCCCCCCCCCCCCCCCCCTTNTTTTTANAAGNTGCNGTANTTTTTTCTTNNTTTTCNTTTTTTTTACN
ANAATNAANTTTTTTTTTTTTTTTANCCCNGNTTTTNNTTTNTTTTTNNNCAATAAATTTTNNNNGGGGG
GTNCTNNTTTTNCGGAACTTTTCNAANANTTNANAATTGNNATTNTTNTCCNNNCTNATNNTNATTTC
CANNCTNCAACCNTTATTANAAAATTATNTTTTCCNAAANAGGTTCNCCTTNNNNACANNNTTNNTAN
NATTTTCNTTTTGTNNAGNTTCTTTTNNAAAAACATTTTTC
GCCTATNTGGAGNTGNATNNCAGTGGCGGCCGCTCTAGAACTAGTGGATCCCCCGGGCTGCAGGTGCT
GACGGACCTGTGGAGCTCCTGGCACTCCTGGACCTCAAGGTATTGCTGGACAGCGTGGTGTGGTCGGC
CTGCCTGGTCAGAGAGGAGAAAGAGGCTTCCCTGGTCTTCCTGGCCCCTCTGGTGAACCCGGCAAACA
AGGTCCTTCTGGAGCAAGTGGTGAACGTGGCCCCCCTGGTCCCATGGGCCCCCCTGGATTGGCTGGAC
CCCCTGGCGAGTCTGGACGTGAGGGAGCTCCTGGTGCTGAAGGATCCCCTGGACGAGATGGTTCTCCT
GGCGCCAAGGGTGACCGTGGTGAGACCGGCCCTGCTGGACCTCCTGGTGCTCCTGGCGCTCCCGGTGC
CCCCGGCCCTGTCGGACCTGCCGGCAAGAGCGGTGATCGTGGTGAGACCGGTCCTGCTGGTCCTGCTG
GTCCCATTGGCCCCGTTGGTGCCCGTGGCCCCGCTGGACCCCAAGGCCCCCGTGGTGACAAGGGTGAG
AGAGGCGAACAGGGCGACAGAGGCATTAAGGGTCACCGTGGCTTGTCTGGTCTCGAGGGTCCCCGCGG
CCCTCCCGGCTCTCCTGGTGAGCAAGGTCCTTCCGGAGCCTCTGTCCTGCTGGTCCCCGCGGTCCCCCT
TGCTTCTGCTGGTTTCTCCCGGGCAAAAGAATGGACTCANTGGGTCTTCCCAGGCCCCATCGGTCCCCC
TGGGCCCTTNAAGGTCCCAACTGGNGAATGCTTGGTCCTTGCTTGGTCCTTCCCGGNCCCTCCNTGGAC
CCCCTTGNTNCCCCAAGGTCCTTCCAAGCGGGCGGGNTTNCNAATTTGANCTTCCTGNCCCANNCCAC
CTTAAANAAAAAGGCTCACCNAATGGTGGCCCGTTNCTANCCGGGCTTNTTAATNCCAATGGTGGTCC
TTNAACNNNACCNTCAAGTGNGGANANCCNCCCCTTAAAAACGTNANCCNANNTAANT
NCANNATCTGGAGCTCCACCGCGGTGGCGGCCGCTCTAGAACTAGTGGGATCCCGCGGGCTGCAGGCA
CCCATCAAGCCCCAAGCTGAAGTTCTATTTAAACTATTCCGTGAACACTATTAATATAGTTCCATAAAT
ACAAAGAGCCTTATCAGTATTAAATTTATCAAAAATCCCAATAACTCAACACAGAATTTGCACCCTAA
CCAAATATTACAAACACCACTAGCTAACATAACACGCCCATACACAGACCACAGAATGAATTACCTAC
GCAGGGGGTAATGTACATAACATTAATGTAATAAAGACATAATATGTATATAGNACATTAAATTATAT
GCCCCATGCATATAAGCAAGTACATGACCTCTATAGCAGNACATAATACATATAATTATTGACTGNAC
ATAGTACATTATGTCAAATTCATTCNTGATAGTATATCTATTATATATTCCNTACCATNAGATNACNAN
CTTAACNANGNNGCNGGGANACACCACCCNCTTGGTAGGGANGCCTTTTTTTNTTNCCNGCCCATAAA
ACGGGGGGGGGGGGGTTTTTANGGAATTTNNCNCGNCTTTNGGNTTTTTTTTNANGGCCTTTTTTTTAA
AAAGGGGCCNTTNTTTTNNTTTTAAAAAAAAAAATNTCGAANGNACNNAATGGGGTAATAAANCCCC
TNNGNCCNCCNAAAAAAGGNGGGGNGGGGNNAAANNTTNGGGATTTTTTTTTATTTTGGGGGGANGG
GGTGGGNCCTCANTTTTGGGCCCGNNAAAAGGCCTTANCCCCNGGAAATNTATTTNGGGGGGGGGNT
ATTAAANGGNTTTTTGGGGCCCCCCCCTTNTGGNAANAGNNGNGGCNTTTCCNGNNNNTNGGGGGCGC
AGGNTNTTATTTTTTTTTTTTTTTNCCCCCNTTTNAAAAAANTTTCCCCTTATNATTCNNCCCNNCCCNT
TTTNNAANAANTTTNCCNAAAAANNTNATTTNAAAATTTTCCCNNNNNTNNTNNAAANCCTTTNAAACCCC
N
NNNNATCTGGAGCTCCACCGCGGTGGCGGCCGCTCTAGAACTAGTGGATCCCCCGGGCTGCAGGGNAT
NTCCTCCCNNNGCCCACAGGCACNNCNNCTATCCGGNACCNTNAGGNNNCNCCNNNGCCTTCCNTGG
NCTTTGAGCNNGGNATAACNTATTTATGATGGNANACCGGGCACNCTTGGGAATGGNCAAAANGGNT
NNGCTNCNNNNTTGGGGCTNGNNGNCGGCTTANNTGAANNNNAGATNTNGNGNCCTNTNGGGACCTT
GGAAANGGGACGNCCCCTTNTCNNATGNNGAANTTANAACTCCTGAACCCCTGGANGNCCNGCNTAN
NAGAAGCCCCCTCAACCNGAAANCCCTGCCCTTTTNNNNCTTTTCCAGCNANANGTNNGGNCGCGAGG
GCCCNTCCTTTCTAATCGACNTTCCAGNNNNNCCNTTCTATTTTNAAAATTTTTTCANNTTNTACNNCC
GCTNNTNNNNTAAGAGAGCACNCACNTTGTCTANGNNTAAACNTCNAACCNTTTAGNATTCTCATAAT
NTCAAGCNCTAGANTTTGATAGGGGNNACAAAAACCCCTCGGGTTNNTAAGATGCCTTCAGNNGGAT
NTCAGGNGGTNNCCTTCTAGTGCNNGGNNAANGGNGNNGCCCACTTTCATTTNCCTGGGGCCCNTGTT
TCGGANAANTTTGTGGGAGGNNTGCCTNTTGCAAAAGGNGATTTGNCCCCCCNCCGCCCCTTCTTGNN
NTNNNAACAACAAGNGNCCCCGGCTTNNAGTGCCCCNNGANGGGGGGGTCCCNNNCGNNTNNCGNCN
TATNNCCCANGANCCCTCGCCACCATNGTNGGTNNCAANAANNNTGGGGGNNTTTTGAAATTTNTCCG
GNCGAGNTCCCNTGGAANNNNTCTNNNGANAAAAATGTTCGTCTCCCCCCNCCNNGTNNNTNNTATGG
GNCGCNNACTTGGTGGNCCCCCCCNNCTTTNNTNCNGCANNCGCCGCCNCTTTNTTTTTTGGANGGAN
TGGNNNATTTTNNNAAGGNTTAANNNGTCCCCC
GANACCCGANANTTANACNNTTNTCTNNANTANNNNTTTTTTTNTATNTNNNNTTNTNCNNNNNTNNN
NNGACGNCCNCNNNCNANNACTNCNANGACNTNNAGTANAGANGAGTGCTNCGCACGNNGNTCTTNN
GNNNGANNCGNGGGTCNNAGACTAGGCCTNCCNTCGNTTTNGGNGNCNNNNTNTGGCAGNTCGCATN
ACNAGTGGGCGGCCNCNTCCTATAATNAGTGCCGTTCCCCCTTTTTTNCNCNAANTGGTATTANCATNT
ATTTTTANNCCNCANNAAANCTCTNTNTACGNCNCTNGGGGACTTNATTAANCTTGNTCNNTTTATCA
NTNGCTNTCCCNCNGTCCTCANCCNCNTATNTNCNCTATCGTCNTCNGTCTTTTNTCTNTTNTNAACTN
ACNCNACACTACCAANNCTNCAANATANTNNCCTCATNATGNANCNNNCTNNNNNCTAGNNTCNTTN
TNNATTTTANTCATTATTCNNNTATNATTANNNCNGAGTNNTTNGGTNTCTTATCATGTNTANTTTAN
NCTNGACATANGNCTNNNTTNATNNCNCANTNCTNTAGNCAGAGCCCATNNTCTCNATNCTNTNTT
NATATNACNNTCNTCGACNTTTNANACNCNACAGTCANNTNNANNNNCNTGNCNCGGACTACCANNC
NCAATAATANATNANANTCACTGCCTNTCNNANNCNCCNGAANGGTTGACNNNTNCCTCGTTNCNCNC
TGANNCNCNCCCCCGTCATANCTTTNANGATTTGCCNNANCAGNANCCCCNCAGNNNACTNANNNNNG
AATANCCTNNTTATTCTCTNNCAATNCTTTCTCACCACANTTNTNTTTATNANATNANTGACCGACAGT
NCCTCGATTNTTCCTNGACTCCCTCTTTTCNACNCNACCAACANTCCNCNTGATACCNCNATAGTCCCT
CTNATNCTNTTATNNNNCACTACCCNTNNNCCTNCCTCNANNNTATCTNCNTTANNCCNNGTCTATNAT
AAGTTCTTTCNTNCNNGTATTCNTCTCNCNNTTCCCCACCCNTCNANCACACNCTANCTNTNNCTTCCC
TANTNNGNTNATTTAGANCNNNNGNNTTATTNNTCCAAGTANTGTNTNTCTTANGTNNCNANCTTNTA
TNCNGTCGTATNCCACTGACCAANGCTCTGTANTANACTCAGGTATCCTNTNATCNCNTAATTATAGNC
TTCTNGCNCTAGTNGNATNGTNTTTAGNATCAACTGCNNCCCCTNCNGATNTTTGCTCTATTATNNTCT
NGTANTNNTGANCNNAACACCACNNCNNTCANTCCTCANCNGANGTGAGNCNNCCATCGATNANTAC
ACTCCGTATCTCNTNNTTNTCNTCTNTGTNCCCCNCTNCTGTNTCNTACNNTCGCCNTACNGTGTNNAT
CTANATCGCTCTCGTATCNNACCTCTNNTNTNCNCCGTATNNNGNNNTCACTCTNATNNTCNATNCAC
ACGTNGNTNTACTAAANCTNANNNNTTAATNNCTCATTAGGTCCTNCNCTATNGTCACCTNCTNCTTAT
GGCTTGTNCTNANAAACGCGTNCCCAACCNTATATGGATCTNAGTCTATTGTGTTNCTCTCATATCATN
NANTTNTNACNCTAGGTNAGTNTCNTATAGNACGANNATTTTNGGNANAAGCNTTANTTNCGGNNNTA
TNNCG
GANACCCGANANTTANACNNTTNTCTNNANTANNNNTTTTTTTNTATNTNNNNTTNTNCNNNNNTNNN
NNGACGNCCNCNNNCNANNACTNCNANGACNTNNAGTANAGANGAGTGCTNCGCACGNNGNTCTTNN
GNNNGANNCGNGGGTCNNAGACTAGGCCTNCCNTCGNTTTNGGNGNCNNNNTNTGGCAGNTCGCATN
ACNAGTGGGCGGCCNCNTCCTATAATNAGTGCCGTTCCCCCTTTTTTNCNCNAANTGGTATTANCATNT
ATTTTTANNCCNCANNAAANCTCTNTNTACGNCNCTNGGGGACTTNATTAANCTTGNTCNNTTTATCA
NTNGCTNTCCCNCNGTCCTCANCCNCNTATNTNCNCTATCGTCNTCNCTCTTTTNTCTNTTNTNAACTN
ACNCNACACTACCAANNCTNCAANATANTNNCCTCATNATGNANGNNNCTNNNNNCTAGNNTGNTTN
TNNATTTTANTCATTTATTCNNNTATNATTANNNCNGAGTNNTTNGGTNTCTTATCATGTNTANTTTAN
NCTNCACATANGNCTNNNTTTGTNATNNCNCANTNCTNTAGNCAGACCCCATNNTCTCNATNCTNTNTT
NATATNACNNTCNTCGACNTTTNANACNCNACAGTCANNTNNANNNNCNTGNCNCGGAGTACCANNC
NCAATAATANATNANANTCACTGCCTNTCNNANNCNCCNGAANGGTTGACNNNTNCCTCGTTNCNCNC
TGANNCNCNCCCCCGTCATANCTTTNANGATTGCCNNANCAGNANCCCCNCAGNNNACTNANNNNNG
AATANCCTNNTTATTCTCTNNCAATNCTTTCTCACCACANTTNTNTTTATNANATNANTGACCGACAGT
NCCTCGATTNTTCCTNGACTCCCTCTTTTCNACNCNACCAACANTCCNCNTGATACCNCNATAGTCCCT
CTNATNCTNTTATNNNNCACTACCCNTNNNCCTNCCTCNANNNTATCTNCNTTANNCCNNGTCTATNAT
AAGTTCTTTCNTNCNNGTATTCNTCTCNCNNTTCCCCACCCNTCNANCACACNCTANCTNTNNCTTCCC
TANTNNGNTNATTTAGANCNNNNGNNTTATTNNTCCAAGTANTGTNTNTCTTANGTNNCNANCTTNTA
TNCNGTCGTATNCCACTGACCAANCCTCTGTANTANACTCACGTATCCTNTNATCNCNTAATTATAGNC
TTCTNGCNCTAGTNGNATNGTNTTTAGNATCAACTGCNNCCCCTNCNGATNTTTCGTCTATTATNNTCT
NGTANTNNTGANCNNAACACCACNNGNNTCANTCCTCANCNGANGTGAGNCNNCCATCGATNANTAC
ACTCCGTATCTCNTNNTTNTCNTCTNTGTNCCCCNCTNCTCTNTCNTACNNTCGCCNTACNGTGTNNAT
CTANATCGCTCTCGTATCNNACCTCTNNTNTNCNCCGTATNNNGNNNTCACTCTNATNNTCNATNCAC
ACGTNGNTNTACTAAANCTNANNNNTTAATNNCTCATTAGGTCCTNCNCTATNCTCACCTNCTNCTTAT
GGCTTGTNCTNANAAACGCGTNCCCAACCNTATATGGATCTNACTCTATTGTGTTNCTCTCATATCATN
NANTTNTNACNCTAGGTNAGTNTCNTATAGNACGANNATTTTNGCNANAAGCNTTANTTNCGGNNNTA
TNNCG
TTNNAAAAANCTGGAGCTCCACCGCGGTGGCGGCCGCTCTAGAACTAGTGGATCCCCCGGGCTGCAGG
GAGTTTCTGGAGCCAGTATCGGATCAATGTGACTGAAGTGAACCCCCTGGGGGCCAGCACCCGCCTGCT
GGATGTGAGCTTGCANAGCATNTTGCGCCCTGACCCGGCCCAGGGCCTGCGGGTAGAGTCAGTGCCTG
GNTATCCTCGCCGCCTGCGTGCTAGCTGGACNTACCNTGCNTCNTGGCCCCGCCAGCCCCANTTCNTG
NTNAAGTTCCGTNTGCAGTACCGTCCAGCANAGNATCCAGCCTGGTNTACGGTGNANCCAGNTGNATN
GAAGGAGGTAATNACCGATNCTGTGGNTGGCNTGCCCNATGTANNGCGGGTNACCGCCCGGAACTTT
NTGNATGCTGGCACCTGAANNTCNTGAAGCCCCAAGGCCTGGGGGANTCCAAGCACTGANCCCTTACC
AAAGGAAATGCCANTTGGGGACCAGCACCCCCCNCCAGACGGAGGAANAACCTTNGGCANACAGCCC
CCGTTTCCCCNAAAGCCGTNCNTTTTTACCCAAACCCANAGCCANTTTGACCANAAAAAACCCCCTGG
AACAAGNGGCCCGCCCTGGCANTTTTTGGNAAATTTTNTTTTTCTTGGNACTGGGGGCTTGGGGCCCTT
GGCATTNGGGGCTTTGGNTAANGGTTGAAACCAAATGAAAANGGGTGGGCCCCCNAAAAATTTNGGN
TTTTTGGNCCCCAAAAGATTTTAAGGGGAAAAACNTTTCAAGGAANCCCCAACCCTNTNAAAGGACCC
TTGGNGGGCCTTTTANTTTNNTTTCCNCCCAAAAGGTGGGGGGGGGGNGGGNGGGGCAAAAAANNAA
AACCCCCGGGGGGGNAAAANAAAATNCCCCCTNGGGGGGGGGGGTTTTTAAAAATGGNAAAANTTTT
TTTTGGGANCCCCCNTTTTTTTTTNNAA
TNNNNCAAANCTGGAGCTCCACCGCGGTGGCGGCCGCTCTAGAACTAGTGGATCCCCCGGGCTGCAG
GAATTCGGCACGAGGCAGGACACAGGTGTCGTGAAAACCACCGTTAAACCTAAGCCAAAATGGGAAA
GGAGAAGACCCACATCAACATCGTTGTCATTGGGCACGTAGATTCAGGGAAGTCTACCACGACTGGCC
ATCTGATNTACNAATGTGGCGGGATCGACAANAGANCANTNGAAAAGTNNGANATGGANNGTGCCGA
GATGGGAAAGGGCTCCTTCAAATATGCCTGGGTCTTGGACAAACTTAAAGCTGAACGTGAGCGTGGTA
TCACCATTGATATCTCCCTGTGGAAATTTGAGACCAGCAAGTACTATGTTACCATCATTGATGCCCCAG
GACACAGAGACTTCATCAAAAACATGATTACAGGCACATCCCAGGCTGACTGTGCTGTCCTGATCGNT
GCTGCTGGNGTTGGNGAATTNGAAGCCGGTATCTNCANGAACGGGCAGACCCGTGAGCATGCCCTTTT
GNCTTACACCCTGGGTGTGAAACAACTATTNGTTGGCGTTAACAAAATGGATTGCACTGGCCCCCTTT
ACCCNTAACAAATACTAATAATTTGTGNANGAAGCTAACNCCTATGTTTAAGAAAAANGGCNTGAGCCC
CTACCCACAAACATCGTGCGAATTTNTGGCTTGGGAANGGGGACAACNTTGCTTNAANGAAANGCTAA
TTTTGCCTTGGGTCAAGGGATGGGAAGGNCACCCGTAAGGGCCGGGAATGCCAANGGGAACCCCCCT
GGTTGAAACTTTTGGAATGGNTTTTTGGCCCCCAANTTTGGCCAAANTGAAAAAACCCTTTCNTTTGGC
TTTTTCCGGGGGNGGNCAAAAAAAANGGGGGGGTTTTGGGNCCTGGCCCCTGNGGGGGC
AAAAGCTGGAGCTCCACCGCGGTGGCGGCCGCTCTAGAACTAGTGGATCCCCCGGGCTGCAGGAATTC
GGCACGAGGAAAAACAAAACGGACCCGGGGGTACATGCGACCCTGAAGTCACTCAGTAGTCAGATTG
AAACCATGCGCAGCCCTGATGGCTCTAGAAAGCACCCTGCCCGGACTTGTGATGATTTAAAGCTTTGC
CATTCTGCAAAGAAGAGCGGAGAGTACTGGATTGACCCTAACCAGGGATCTGCTGAAGATGCAATCA
AAGTTTACTGCAACATGGAAACAGGAGAAACATGTATTTCAGCAAATCCATCCAGTGTACCACGTAAA
ACCTGGTGGGCCAGCAAATCTCCTGATAATAAGCCTGTTTGGTATGGTCTTGATATGAATCGAGGATCT
CAGTTTGTTTATGGAGACCACCAGTCACCTAATGCAGCCATTAGTCAGATGACCTTCTTGCGCCTTTTA
TCGAAAGAAGCGTGCCAGAACATGACCTACATCTGTAAAAACAGTGTAGGATACATGGATGATCAAGC
TAAGAACTTGAAGAAAGCTGGGGTTCTCAAAGGGTCAAATGACTTAAAAATCAAAGCAGANGGGAAC
CGTTAGATTCANATACATAGTTCTTCATGAATTCTTGCTCTAAACGAAATGGNAACGNGGGCAANACC
NTCTTTGAATNTANAACGCAAATGNGGCACGCTTGNCCATCATANATCTTGGCCCCTGGGGATGTTNG
CAGTACCANANCCAANAANTTTGGCGTTGAAATTNGGGCCCANTTTGNTTTGGGTTAAAGCGAAGNCC
GNNATCCCTTGGNCANTGGGCCCCCCCCCCCTTACCCTTAGNGGNCCCCCCCCCTTTCCCCAANAANTT
TTTACTGGTTTGAAACCTGAACCCTGGNAACCNTTTGAAANAANGGGGTGGANTTTGGCCTCCANCCC
NTN
TTGANCNCNNCTGGAGCTCCCCGCGGTGGCGGCCGCTCTAGAACTAGTGGATCCCCCGGGCTGCAGGA
ATTCGGCACGAGGNNCCNNNCNACNNTTNNTCNATGGGGGGNNCCTNGGTCAACGCNNGGTTCATTN
TANNCNNGCATGANCNTANNANCACCCGGCNTTGGTGACCGCNNNCCCCNCTNACACGTTGAANATC
ATTANTATTTCTATCTCACACANGNCTCATTCGAAGCTAGCCCTCGCCGAGTNTGTATTNNTCNANTCA
NNTNTTNATTATANNNNNNCTTTTTTNTANTNCNAACGNCANNCNACTCNGNTTNANTTTTTANNCAG
NGNACTATACCTTTNNNNACCACNAAAGGTTNATAAGACTCAACCNNANNGGNGTACCTGGNGACNA
TNTCNNNNNTTNNCAGCATNGAATTTATACTATAATGTTTCAGTCAATATAATTCCACTTAATAATTAG
NGAAAATTTCATATTATCCTAAGTATTACAGNATTAGTCCATTTTTTTCTTCTCCTAAATGAGGAGTGG
TTTATTTCCTCNGCGGNTTATATCATTAACTTTCTTTAATTCTTTTNTGNGCTGNGNGNAATATNGATAA
NATTACNGACCCCCTATAAAATAAATGNAAATTAAAAAAAAAAAACCCCCNCCCCCCGGGGGGGNCG
GGAANNNNNNGGTNCNCNCTTAANNTGGNGNNNTTTNNAANCCCCNGNTTNGGTTNNNCCCCCNNTTN
GGNAANNNNCCCCCNTTTNNGCNNNNTNTNGNTNNTTNNCNNNNGGNNCCGNAAAAANNNCCCCNGGG
GGGNGGGNCCNCAANTNGNNNNGGNCCCNNTTTTNNGNCCCCNNNTTTTNNAAAANNANNNNCCNNN
GNTNNANNNNTTTGGGGCCCCCNNNTTTTGGCCCNNGGGGCCT
GCCTATANNGAGNTGNATNNCAGNGGCGGCCGNTGTAGAACTAGTGGATCCCCCGGGCTGNAGGAAT
TCGATATCANGCTTATCGATACCGACGACCTCGAGGGGGGGCCCGGAACNNANTTNNCCCTATNGTGA
GTCNNATTTNCAAGAATAAACGGATCTTGAATAAAAAAAAAAAAAAAAAACCNGGGGGGGGANCGGN
NCCCNATTTCCCCTNNNAGGNGNTTTANTAAAAAGGACTGTNACTGTGNNNTCAGTGAACGNNNGCT
NANANTCANCCNNCTNACNCTCCTGTACCACANGNGNTATTNAACTNGGGGNTNTTATTAAGACTTTT
NTGANNACACTNNTNNTNNTCNTTTGNAANCTTNTCCNNTGCAGTGNTGTCAAATATNNNCNGNNNA
ATTTCTCTAAANNNNANACNNCTNCTNNTTTATAACCTANTTNCNNCCANNGNCNANNTTTNNANCCN
ANNNGNNNNAATGTAANTTACNANNCTTNGATAAACCCGTNATNNANTANACNNTNACCAAANCTGN
NATNGANGTGNCTAAAAAANATNATNNCNNNNAAANGTTTAGTTATTTAANTTNANTNNGNCNCNNA
TTNTTTNTTNNNATNNATNTNNACCCCNNNGGTCGNNANTNTNTNTTTTNTTTTTNCTANAATTNCAAT
TTCCTANTNNNNAANNTNNNANNNNACNATNTCCCTNANANNTNCAANTGANCTTNTGAAAANTNTA
ANNANNNATNCANTNCTCNANNANCTANANNNANNTANGGAAATTNATNTNGNNTGCNTTTTTATTA
NNTTTNNTTNTTNTNATTNTTCNACCNAAATCTNATATTNNCTTTNNTAANNNTCTANNNTTTTTCAT
NTTTNACTCAANCNTATNANTCNNACGNAATNCNNCNTNGNTNCANTANCCATNNTTNTNNCTANTAT
TNTCTNCAANANNCNGCAATCANCATCTGNANATCCTANCNNGCTGTNGTCATCTACATACNNTNNAT
NTNTANNTGNCTTNANANCGTANTNCAANTCANTCTTTCNNTTANTTNTNTNNNTATCTCATNTTNAC
T
NNNNACTGGAGCTCCANCGCNGTGGCGGCCGCTCTAGAACTAGTGGATCCCCCGGGCTTGAGGGCCC
NGGCTGGATCAGCTGCTGGGGACACTCTGACCCCTCAATGCGGGCCTTCCTGCTCTCCCTGGCTGCCCT
GCGTGACAACCACACCCACTCCGACATCCAGGTGAAACTCTTTGTGGTCCCTGCGGACNATGCCCAGG
CCCGAATCCNTTATGCCCGCGTNAACCATAACAAGTACATGGTGACTGAACGGGCCACCTACATCGGA
ACCTNCAANTGGTNTGGCANCTACTTCACCGAGACGGCNGGCACCTCTCTGCTGGTGACACAAAACGG
GCGGGGTGGCCTGNGGAGCCAGCTGGANGCCGTGTTCCTGAGGGACTGGGACTCCCNTTATAGCCATG
ACCTTGACGCTGCCGCNGACAGCGTGGGCAACGCCTGTCGNCTGCTCTGAGGCCCGGTCCGATGGCCA
GGCCAGGCCTGCAAGGCCCTGGTGTTCCGGGTCTCCATCCCCTGTCCCTGTGCCCCCCGNTTTTGTCTG
ACCCGTTGTGACTNCAACAGGCTCCCNCTTAGCAACCACCCCCACCCCAGCCTTTCATTTCTACCTCCA
CCCCCNNTTGGNCTGATGCTTGTGGGCCCCAAGAAGACCCANCCGAGCTGNGGGAAGGGCTCAATNC
CCAAAAANAAAAGTGGGGGGTGCATGCTTNGCCTTACCTTCCTGGGCCAACCCCANTTTCCAGGGCAA
AGNAAGGCCCCAGGTNATAAATAAAAAAGTCAAATTACTTTGGNTTTNAAAAAAAAAAAAAAAAAAA
ANTNGGGGGGGGGGCCCCCNNNCCCANTTTTCCCTTAAANANGGNNNNNNNNNAAANNNNNNNNNN
NNNNNNNNNNNNNNNNNTNNTNNNNANNNTNNNNANNNNNNNNNNANNNNNNANNNNNANNNNNN
NNNNNNNNNNNTNNNNNNNANNNNNNNNTNNNNNNNNNNNNNC
GCCCTATCTGGAGCTNNANCNCNGTGGCGGCCGCTCTAGAACTAGTGGATCCCCCGGGCTGCAGGAAT
TCGGCACGAGGCTGGNATACAGGAGGCCCTCCTACTTCTCTGGGAACTGCCGGACGAATTAAAGTGGG
GNACTGGAAGTGCTNTAAGGGAAAAAAAGANCNCGCGTCTTCAAATTCATTTGAAGANCTNAATAAG
NCTTTCCATGAAGCTGGTTGNGGGGGGNGNGGCANANTCCTACTGNATTCCCGTANANNGGGCTGNTA
TGGGNGCGGCCCACTTATTANAACANANAGGCCGCTGNCCGAGGNTNACTTCCACNCNCCTTTTCCTG
GCTTGAGGCNCCCAAACTNCTCAAANCTANACNGGCTGCTTCTNANAATTTCANGGAGGCGGGAGAA
GTATAACCCCGCTGGGGAAGAGGGGGCCACATGATGTGGTTCANCTGGAAGACACGCACACCACATG
CTCTCTGGGATNCTCTGTACAGAACGGACNACACTTGNNTCNACGCCNANNGTCACATNTCGACTGNG
CAACTCTTANNATNNAAGGACCCCTCGGGCCCNGTGGCTGTTNATATGCNCTATTTTGGAAAAAAAAC
ACAAATTTGTAGGAAATACCNNTTTTTTTTNACATTNNTNAANCAAGCAAACCTGAGACCCCGGGTCA
GGGTTTTTTCATTTNAAAAATGTGNTCTGCTTNTTATNGGGGCCAAAAAGTTGAAACAGCNTTNCCCCT
GTTTTTCCCCCATCANNAAGGGNNACAACANCCANGTTCGNCCTTCNTTTTTTTTTTTTTCCCCCCACCC
ACTTGGCNACCCCCCAAGCNGTNGTTGTTNTAAANTGTTNNCCCCCCCCCCCCCCCCCCCCCCCCCGCC
CCCCNTTTTNAANGGTANCTCTCCTNCTTCCACGCATTNNNTTNNNNGGANGCGGNTTGNNTCTTTNTC
CGCANNCATTTTTCNNNCNNCACACTNCATCNATANNCTNCCG
TGAGNCCNTNANNGNNTCGTATTACAGTGGCGGCCGCTCTAGAACTAGTGGATCCCCCGGGCTGCAGG
NCNAGTNNTNGTTTTNTTTTTNTNCCAGNGAAGGNNTTTTTTTGGGCCNGGGCNTNCTNNTCCNAAAA
ANNAATNGGNTNCATNGCTNCCCNAANCGGNNGTCNNCCAAAAANGCGNATGTNCTTNGNAAGGGNN
AACTTNTNNTANAAAAACCCCNNNGNGGTNGGNAGGGNNGACCGAAAGGNNANAAACAAACTNGTA
NGGCCNNGNCNTCGGGNNGNTNCCCGGTTTCANGNTTNGNGAANNTGGNATNAAACCTNCNGNCCTT
CCNANTTNTGCCNNTNTGAACNCNNGGGGATTTGNGGAGAGANNCCCACNNGACNGTTTNTTTNNNA
GNANNGNCAANCACNGNGCGACANNTNTACNATGTNGAAANCCAANACTTCTNATACNNNNCCCTT
TTNTCCCTGAANGGGAGAATAAGGGCNTTTCTTTGTGGNANAAANACCCTNATAAAGCNCCCTGGGAC
NCAATGTTNTGGNNCCCTGGNCCNNCNGCACAAAAANTTNNANNGGNNTANCNNNTCCTNNTNCCCA
NANTCNTANAGGGAATTTCNTTAAAAATNTCTGNNNCTNAANTNNTTCNCAAAGTTNTTTTAAANATG
NCAGNCNNACNGTTAANNCNCTCGCCTTNTTNTCNCANGAACNNTTTGGNCCNNNNCNCNNGNGTAT
AAANTTCGTGNANNNCNGNGNANCAAGTTTTTTTNAACACNNCNNNNATTATATATNGGNGNGGGCN
NANTCCNTTTCNCNGNNANCTTNTNNAGNNNGTTNACTCACTNNTAACCNNNNGTCNNGNTNGTCCT
CTTTATGNCANANNCAAAATCTCCNACNCTTCGCTTGAATTCNACCNNAAACCTTTTNCCTNCAAATTN
CCTAATNCTCNTGGTTNTNTCACTANAATAACATNNNNANAAANNNNCTCNCGNTTTTATCANCNCCA
TCNCNTGAAGGCCG
TTTNAGCCTATNNGGAGNNGNATNNCAGTGGCGGCCGCTCTAGAACTAGTGGATCCCCCGGGCTGCAG
GTCCGAGTTTTTTTTTTTTTTTTTTTNANNGGGNNGNCTTTTTTTGGGGGGGGGNTGGNNCCCCNNAA
AANNATGGGNNNCANNGGAANCCCANCCGGGGNCNCCCCNAAAANGGGGNTNNNCNTNNCCNGGGA
ANNTNTTTTTTAAAAAANCCCCNNNGGGTNGGACNGGAAANCCCNAGGNTNAAANCNAAACNGGA
AANCCNTGGGCNNNGGNNGNTTNCNGGGGGCCCGGNTGGGGNATTGGGNGGNAAANCNTNNGNNGG
GCCCGGGTNNCCCATTTNANCCCCAGGNNTTTNGNANGAANAAAATTNGNCCCCTNCNTTNGANGG
GGGNCNANTNTGGGCNGGCNCCNNCCAGGGNTGGGGGNCNNNNCCCGNAAANAAANNCNGGGNNGG
CCNGGGGGGNAGNGCCTTTTNGGTTNAAAAAACCTNTCTNNNNNNCCCNGGAAAAAANGNTNNGGCC
CNNGGCNNNACCCCNANCNTNGGGGGGGGNCCCCCTCCNGGNCCCGGGGAAAANGGTTTTCCCAAAN
NCNNTTCCCCCNTTGTNGNNCANTTTTGGGGAAGGTNCCANAATNGGGGCCCTTNTTTTTCNCCAAAN
ANGGNNGNTTCCCNCGGGGTTATTTTTNTNGTGGGGNGGCCCCCCCTCTTTTAAGGGGCCCTATTAN
AGACNGGGGGGGCCCCCANNCCCCCCCNGNGGNTTTTTTTGNGGNNGAANNNCCCCCTTTGGNCCCCC
NCGNNGGGNGGNGCCTTTTTGGGGANCCACCCCCCCCCCNNGCCTTNTNTTNCCCCCCCCAAAATTTTT
TTTNACCNNAACCCCCCCNNTTTGGTTGGGNGGGGGAAAAAAAAAATTTTNTNGGGNGGGGGCNTNG
TTTTTTTAACCCCCCCCGGGGGGGG
TTNANAAAATCTGGAGCTCCACCGCGGTGGCGGCCGCTCTAGAACTAGTGGATCCCCCGGGCTGCAGG
GAGTTCTGGAGCCAGTATCGGATCAATGTGACTGAAGTGAACCCCCTGGGGGCCAGCACCCGCCTGCT
GGATGTGAGCTTGCANAGCATNTTGCGCCCTGACCCGCCCCAGGGCCTGCGGGTANAGTCAGTGCCTG
GCTATCCTCGCCGCCTGCGTGCTANCTGGACNTACCNTGCNTCCTGGCCCCGCGAGCGCCANTTCNTGN
TCAAGTTCCGTTTGNAGNNCCGTCCAGCACAGNNTCCAGCNTGNTNTACGGTGGACCCAGNTGGATTG
AAGGAGGTNATNACCGATGNTGNGGTTGGCNTGCCCNATGTANGGCGGGTCACCGCCCGGAACTTTTT
GGATGNTGGAACCTGAAGNTCNTGAANCCCCAAGGCNTGGGGAANTCCAAGCANTGACCCCTTACNN
AAGGAAATNCCANTTGGGGACCANCACCCNCACNCAGACGGAGGAAAAACTTTTGGGCANACAGCCC
CCGNTCCCCNAAAGCCGTTCNTTTTTACCCANACCCAAAGCCANTTTGACCANAAAAAACCCCCTTGG
AACAAGGGGCCCGCCCCTNGCTATTTTTNGGAAATNTTTTTTTTTTNTGGAAACTGGGGGCTGGGGNCC
CCTGGCNTTTGGGGCNTTTTGGTTTAAGGGGTTGAAACCCAATNTGAAAAGGGGNNGGNCCCCCNAAA
AATTTTGGGNNTTTTTGGNCCCCAAATNTTTTTNAGGGGGNANAACTNTTTCNCAGGAANNCCCCAAA
CCCTTTNAAAANAACCNTTGGGGGGCCCTTTTTATNNTNTTTTNCCCCCCCAAANNNNGNGGGGGGGG
GGGGGGGGGGGAAAAANAAAAANCCCCGGGGGGGGAAAAANAAANNNCCCCTNGGGGGGGGGGGT
TTTTAAAAAGGGNAAAAATTTTTTTTGGAANNCCCTTTTTTTTTNNAA
AACAAANCTGGAGCTCCCCGCGGTGGCGGCCGCTCTAGAACTAGTGGATCCCCCGGGCTGCAGGTT
CGGCACGAGGCACGGCTTTTCCCCCCTCTGACATCGTCTGGGGTCCTGATTCTGTCCTCGTCTCTTTGA
AAGCCCTCTGATGAAACAACGGCCCCATCGCTGCCCTACAAGGGTTTCCATTTCCCCCAAGTCGTCTAT
GCATTTCTCTCACCGTTTTCTGCCTGATTCCTAGAGAAAACGGGCATCTCATGTTGCACCCTCTGAGAC
CCCCCCTTTCAGAAAATGCTGTGCATTTTGGAGACGTGAGGATTTCGTCCAAAGTTCTGAAAGCATTTG
CGGAGAGCTCGTTAGAAAATAAAAATGGGAAAAAGCAAAACGAAAAGGCGGTGTCCCCCCACGTCCC
TTTCCACTGAGTAAGACCTAGCTCTTTGCGATTGTGCAATAAACTACTTGCCGTGAGAGCAGGTGGCC
ACGCTCCTGCCCAGACTACTAAGTGACACAGCACANGACGGCGGGCTGCACGGGGGCGTCTCCCAAAC
GCCTNCCATGAGCTCAGCTTCCCCCACCACCACCACCAAGCACCGTGTCTTGGCGGNGTCTTTACATTT
TAACAAGTATTTTATTTTATCTGNAACCGCACTAAGTCTTAAGGGGTTTTAAAAGCTTCGGTTGGTTCA
TACTCACAATCAAAACTTTTTTTTTTTTTTTAAACCCCCCCNANGGNCCCCANTTGGCCCAAAAAAAAA
AAAGCCTTTTTTTTTTGNCCAAAAANACTTTTAAAAAAAAACCCTTTTTGNGGGGGGGGGTGGGNNC
CCCTTTCCCGGGNAANAAATTGGGGNNNTNGGCATTTTGGGGCCCCCGGCCCCGGGGAAAAANAAA
AAAACGTTTTCCCCCCCCTGGGNNNNGGGGGGGGGGGGGAAAANCCCCCGGGGNCCCCTTTTTTGGN
NNTTGGGTNAANCCCAAAANAN
NAAAAGCTGGAGCTCCACCGCGGTGGCGGCCGCTCTAGAACTAGTGGATCCCCCGGGCTGCAGGCAA
GTGCGGGGGCCGCCACGGCAGCCTCCCCAGGGGCAGCCCTTGCAGGGCGGCATGCTGGCCTCCTTACG
CCAGAGCGGCTCCGACCTGGCCTACAAGCTGTGCCACAATGAGTGGGTAGTGGTGCAGAGCTCCACGC
ACCTGGTGGACGTGGCTGGCTGCGCTGTGGACGTGCAGCTGCTCGAGGACTCCCACCAGGTGAGGAAA
GACGGCTGCAGCTGCAGCTGCTACTTTCAGCAGCGGTACCACCTGCCGTGCCGGCACATCCTGGCGCT
GCTGCACACCAGCCAGAGGCCTGTGAGCGAAGCCATGGTGTGCCGCGGGTGGCAGAAGAGGTACCAG
CACCTCCTCGGGCCCTGCGGGGAGCTCCGGGACCCCATTCTGATCCCAGGAGATGCAGGCCAGCCAGG
GGAACCGGGACGGAACGACATGATTCAGGACCTAAGCAGGGAGCTTGCAAATCTGCTGATGCAGAGT
GAGGGACCAGAGCTGGAGGAGCGCTGCTCCACGCTGCGCAAGATTGTGGACATTNTGGGCGGNCCCC
CGCCAGCCTTCTGAGCCCAGTCACCAANCCGANGGAATTTCANGGATGTGGGCTTGNCTNCCTTTTCTT
NTGGGGAAAGCANGAAGGAAGGGGANGGACTGGCTTCTTGCTGGCACCCACCATTTCATGGTTGAAA
CCCCTTTGCCCTTGGCATTGCTTAGGNCGGCAAGCCCCNTTTTCCNTTACAGNCCNAANAAACTTNAAA
GGGGGGCCANGAAATTTGCCTCGGGGGTCAGGGTTTTAAANCAAAGGTTTTTTTNGGGGNNGGGTT
AAGGAAAAAGGGTTNCCAAAAAAAAAAAAAAANCCCCTTTTGGGGGGGAAAANCCCNTTTNAAAATN
GGNCCCCNNTTTTTGGNCCCGGGGTTTT
AAAGCTGGAGCTCCACGGCGGTGGCGGCGGCTCTAGAACTAGTGGATCCCCCGGGCTGCAGGGAAAA
AAAGAGAAAAAATTTAAAATAAAAAAAAAGAAAGAAACCTCCAACGTATTTTATCACTACCTATAGG
AAGAAATCCTGCTTTGAGAGTATTTGTAATGCGGTTTTGTTGTCGTTTGTTGCTGCTTATTTCACTAAGA
AAAACCCAACAACTGAGACTGCCTAGCCCGCCGGTCCTGTGCGCTTTTATTGTGCTTCTAACCCCAGTA
GAGTAGAACTAAATTGCACTGAATGTATAGTTAACTGTCTTGAATCCTGTGTTTATGCAATGTGGTCGA
AAGAAAAACAACGTTAAAAATATATCTATAATAATAATTTTTGGTCATTCGTCTTTATGTCCAGCTATG
AATGTAGATTTTGTGTCCCGGCAGCAGCTCTGTTTCTGGTCGAAGTACTTTGTATTGTATACCGTGAGT
CATAATAAAAAAAAAAAAAAAAANCTNGGGGGGGGGCCNGGNCCCCANTNCCCCNTATNGGGAGCG
GTTANANNNNNNNNNNNNNNNNNNNNNNNNNCNCCCCCCCCCCCCNNNNNCCNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNN
AAANCTGGACTCCACCGCGGTGGCGGCCGCTCTAGAACTAGTGGATCCCCCGGGCTGGAGGCGTGGGT
AGAGCGCCTGAAGGCCTCAGGGACGCTTCGAGAGTATAAGGTGGTGGGGCGCTGCGTGCCGACCCCC
AAATGCCACACGCCACCCCTCTATCGCATGAGAATTTTTGCGCCTAATCATGTTGTTGCCAAGTCCCGC
TTCTGGTACTTTGTGTCTCAGTTGAAGAAGATGAAGAAATCCTCGGGGGAANTTGTCTACTGTGGACA
GGTGTTCGAGAAATCCCCCCTGCGAGTGAAGAACTTCGGCATCTGGCTGCGCTATGACTCCCGCAGCG
GCACCCACAACATGTACCGGGAGTACCGGGATCTGACCACTGCCGGCGCCGTCACCCAGTGCTACCGA
GACATGGGCGCCCGGCACCGAGCCCGGGCCCACTCAATCCAGATCATGAAGGTGGAGGAGATCGCAG
CCAGCAAGTGCCGCCGACCCGCGGTCAAGCAGTTCCACGACTCCAAGATCAAGTTCCCACTGCCCCAC
CGGGTCCTCCGTCGCCAGCACAAGCCACGCTTCACCACCAAGAGGCCCAACACCTTCTTTTANGTTCA
GGCCCTCTGTCCCCAGGGCTGCACAAATAAAACTTAGTGGGAAAACTGNNNNNNNNNNNNNNNNNNN
NNNNNNNNNNNNNNCCCTGGGGGGGGGGCCGGGCCCAATTTCCCCTTTTANNNGGGGNNNNNNTNNA
NANNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNC
GCCTATANGAGNNGNATNACAGTGGCGGCCGCTCTAGAACTAGTGGATCCGCCGGGGTGGAGGCTG
CGAANGCCAGGGAGTTCAAAGAATGCGTGGAGTGCTTCGAACGCTCTCTGAAGATTAATCCCATGCAG
CTCGGAGTGTGGTTTTCTCTGGGCTGTGCCTATCTGGCCTTGGAANACTACGGAGGTTCAGCGAGGGC
ATTTCAGCGGTGTGTGACTCTGGAACCCGACAATGCTGAAGCTTGGAACAATTTATCAACTTCTTATAT
CCGCTTAAAACAAAAAGTAAAAGCTTTTAAAACATTACAAGAAGCCCTCAAGTGCAACTATGAACATT
GGCAGATCTGGGAAAACTACATTCTCACTAACACTGATGGTGGAGAATTTTNTGANGCCATTAAAGNT
TATCATCGGCTCGTTGTNCCTACGANACNACTACGATGNTCCNGNNTTNNNAANCTTGNTNGGGNCN
GNCGANTGATNGNNATGGACCNGNCCGATNTGGCTNAGNANGAACCCGNCCTCNNCAAAGAAAGNTN
CCAGGAGNTNNTTTNGNNNAGTGTCTTTCNCNTAACTGGNNNNNNGNNGGGGAAANTCACGGNNGGG
GNGTTNGNCTGCTTCANATTNGAAANGGCCGCNGGGNTNANTNACCCTCCNTGANATAAAAAAGGGG
NACNCCCCTTNCANTNTCTTANTGNNANNCATNGGNNGNCCCNACCCCCCATNTTTNGGGGGAAAAA
AAAAATAACTNTTTTTTTNATNTAAGGGNNNNANANANCCNTANCCCNTTCGCACCNNTGGGCTTTTT
AATTTNAAANAAAAANAAAAATCCCNTCCCNGGGGGCCTTTTAAANATATNCGNGTTTCCAGNNNCCC
ANNCNANTTTTNGGGNGCTCTNNTTTTTTATAACANAAAAAACNNTTTTNTNCANTANTTGCCAANTG
GGAGAAAAATANNNCGNCGAGTATTNTTATGNTCNAAAAAAAACCNTNATTTTNNNCCCCTCTTTTNN
NTNCNNNNNTNTNNAATANTTCTTCNTNCNCTNTTCTCANCANNCCT
NCANNANCTGGAGCTNCACCGCGGTGGCGGCCGCTCTAGAACTAGTGGATCCCCCGGGCTGCAGGTA
GTAAACCCAAGCCCTTGACCTCTTACAGGGAGCTTTGGTCTGGCCCTTCTTAATAACNNTCCGGGCCCN
ANCNTGGGNATTNAACTTTNANCTTNANGGNCCNGGTTANNANTGGNCCNACCACCAANNTTNNNAC
CATNNNNCNANGGAGNNCAAAAGGTNNTCNNAAAANNNCCNTTCNAGGGNNCCNTNCCCCANCTGGC
CNAAAAGGGCNTCGGTATGGNANAANTCTTTTTANTANNTNCNAAGNACNANTCTTTANCGGANTTTT
CTNNANNTTTTTACNANTNAGGCNTGGCCCCAACCGTNGACNATAGGNGGTNGNNNACCCCAACANG
GCTTCTCACCACATATAACCCCCTATANAAGTCCAGATGNGCACACAGACTCTGTGTCTATATGNGNCT
TCGGNGAGTTCTATANACACTGAGNCCNNATATATANTATNATANAGGGANACGCGAGCGNATATAN
CACNCGCNNTNTNTANCNNNATCANNTTGAGAGTANNNTNCACTNTNNNACNNNNCTGAAAACNNTN
NNANACNNNTTNCTATATCCNACGGAGGANTACANGNCATCATTTTTGTANACACAGGGGTTCACG
GCGNTCTCGACGNTCATNTGGGGGCACCNNTCTTTAATAAGTGNGCTGCTTCCCGCCGACAAAAAAAA
ATATTTTACTTCTTAACCACCACTTCTGCTNTTAAGACGCNGGCCNGATGNTGNCGTTNTTNTAGACGA
NGTAGGGACGATTTCCTCCATGATGCTATCTATTGANGANGNGGGCTTNAANAANNNNAAAANNNNN
NNNNNNNNNNNNNNNNNNNNNNNNNNNNNNNNNNNNNNNNNNNNNNNNNNNNNNNTTNNGGGGGG
GGGNGCCCCCN
TGAGNCCNTNANNGNNTCGTATTACAGTGGCGGCCGCTCTAGAACTAGTGGATCCCCCGGGCTGCAGG
NCNAGTNNTNGTTTTNTTTTTNTNCCAGNGAAGGNNTTTTTTTGGGCCNGGGCNTNCTNNTCCNAAAA
ANNAATNGGNTNCATNGCTNCCCNAANCGGNNGTCNNCCAAAAANGCGNATGTNCTTNGNAAGGGNN
AACTTNTNNTANAAAAACCCCNNNGNGGTNGGNAGGGNNGACCCAAAGGNNANAAACAAACTNGTA
NGGCCNNGNCNTCGGGNNGNTNCCCGGTTTCANGNTTNGNGAANNTGGNATNAAACCTNCNGNCCTT
CCNANTTNTGCCNNTNTGAACNCNNGGGGATTTGNGGAGAGANNCCCACNNGACNGTTTNTTTNNNA
GNANNGNCAANCACNGNGCGACANNTNTACNATGTNGAAANNCCAANACTTCTNATACNNNNCCCTT
TTNTCCCTGAANGGGAGAATAAGGGCNTTTCTTTGTGGNANAAANACCCTNATAAAGCNCCCTGGGAC
NCAATGTTNTGGNNCCCTGGNCCNNCNGCACAAAAANTTNNANNGGNNTANCNNNTCCTNNTNCCCA
NANTCNTANAGGGAATTTCNTTAAAAATNTCTGNNNCTNAANTNNTTCNCAAAGTTNTTTTAAANATG
NCAGNCNNACNGTTAANNCNCTCGCCTTNTTNTCNCANGAACNNTTTGGNCCNNNNCNCNNGNGTAT
AAANTTCGTGNANNNCNGNGNANCAAGTTTTTTTNAACACNNCNNNNATTATATATNGGNGNGGGCN
NANTCCNTTTCNCNGNNANCTTNTNNAGNNNGTTTNACTCACTNNTAACCNNNNGTCNNGNTNGTCCT
CTTTATGNCANANNCAAAATGTCCNACNCTTCGCTTGAATTCNACCNNAAACCTTTTNCCTNCAAATTN
CCTAATNCTCNTGGTTNTNTCACTANAATAACATNNNNANAAANNNNCTCNCGNTTTTATCANCNCCA
TCNCNTGAAGGCCG
TGAGNCCNTNANNGNNTCGTATTACAGTGGCGGCCGCTCTAGAACTAGTGGATGCCCCGGGCTGCAGG
NCNAGTNNTNGTTTTNTTTTTNTNCCAGNGAAGGNNTTTTTTTGGGCCNGGGCNTNCTNNTCCNAAAA
ANNAATNGGNTNCATNGCTNCCCNAANCGGNNGTCNNCCAAAAANGCGNATGTNCTTNGNAAGGGNN
AACTTNTNNTANAAAAACCCCNNNGNGGTNGGNAGGGNNGACCCAAAGGNNANAAACAAACTNGTA
NGGCCNNGNCNTCGGGNNGNTNCCCGGTTTCANGNTTNGNGAANNTGGNATNAAACCTNCNGNCCTT
CCNANTTNTGCCNNTNTGAACNCNNGGGGATTTGNGGAGAGANNCCCACNNGACNGTTTNTTTNNNA
GNANNGNGAANCACNGNGCGACANNTNTACNATGTNGAAANNCCAANACTTCTNATACNNNNCCCTT
TTNTCCCTGAANGGGAGAATAAGGGCNTTTCTTTGTGGNANAAANACCCTNATAAAGCNCCGTGGGAC
NCAATGTTNTGGNNCCCTGGNCCNNCNGCACAAAAANTTNNANNGGNNTANCNNNTCCTNNTNCCCA
NANTCNTANAGGGAATTTCNTTAAAAATNTCTGNNNCTNAANTNNTTCNCAAAGTTNTTTTAAANATG
NCAGNCNNACNGTTAANNCNCTCGCCTTNTTNTCNCANGAACNNTTTGGNCCNNNNCNCNNGNGTAT
AAANTTCGTGNANNNCNGNGNANCAAGTTTTTTTNAACACNNCNNNNATTATATATNGGNGNGGGCN
NANTCCNTTTCNCNGNNANCTTNTNNAGNNNGTTTNACTCACTNNTAACCNNNNGTCNNGNTNGTCCT
CTTTATGNCANANNCAAAATCTCCNACNCTTCGCTTGAATTCNACCNNAAACCTTTTNCCTNCAAATTN
CCTAATNCTCNTGGTTNTNTCACTANAATAACATNNNNANAAANNNNCTCNCGNTTTTATCANCNCCA
TCNCNTGAAGGCCG
TTNNANAAAAGCTGGAGCTCCACCGCGGTGGCGGCCGCTCTAGAACTAGTGGATCCCCCGGGCTGCAG
GAATTCGGGACGAGGCTCTCTTTAGTTCTTTGTACGGAGGTGAAAAAAATAAAAAGATCCTTCAAAAA
TGGCAATGGTATGTGAATTCCTCAAGCAGGCCTGGTTTATTGAAAATGAANAGCAGGANTNCNTTAAA
ACGGTGAANGGATCCAANGGNGGTCCTGGGTCAGCAGNGANCCCNTATCCTACGTTCANTCCNTCCTC
GGATGTTGAGGCCTTGCNCAAAGCANTCACAGTTAAAGGNGNGGATNAANCACCCNTCNTTGAANTT
NTGACTAAAANAACCANTGCACAGNGTCAGCAAATCAAANCGGCCTATCTGCAGGANAAAGGAANGC
CCCTGNNTNAAGTNNTGAANAAGGCCNTCNTCGGNCACCTTGAGGANGTNGTTTTGNNTNTATGGAAA
NCTCCANCCCANTTNGATGCCNAAAAGCTCCGCGCTGCNNTGAAGGGCCTTGGAACTGATNAAAACCC
TNTGAATGAAATTTTTGNCTTCGAGAACTTCCCAGAGAGATCAGAGAATTTANCAGAGTCTNTAGAAA
AAAACTGAAGAAAGATNTGGCTAAAANACATCGCCTTAAACCCATTTGGANAATTTGAAAAGGNTTTT
GTTTGCTNTTTCTTANGGGGGACCCGACCCCAGGGACTTTGNTGAAAAANAACAANTTGGGTTTTTTN
AAANNCCCGGGCCNTTTTTTTAACCCCGGGAAAAAAAAAAAAAGGGGCCCAANNGGGAAANNGGTTN
CAATTACCTTTTTTTAACCCCCNGGGGGNTTTTNCCCNTTTTTCGGGNAANGGGTTTTAAAAAAANTTT
TCCCCNNTTTTCCCNCCCTTTNNNTTTAANANAANTTTTTTTGNCCCCCGGGGTTTNAAAAGGGGGGC
CCCTNNAAAAANNCN
TNNAACAAAAGCTGGAGCTCCCGGCGGTGGCGGCCGCTCTAGAACTAGTGGATCCCCCGGGCTGCAG
GAATTCGNTATGAAGCTTATCNATACCGTCCACCTCTANGGGGGGCCCGGTACCCANTGNNCCCTATA
NTGANNCACATTACAGCCCGGAGGAATGGTAGATGGCANGTGTTCTTGTTTTTAACCAATATGAGGGA
TGTCCTGAATTTGGTAGAACAACTGTGAGACAACTTCAGCAAGCTTTGTAAACAGGTTTTATATTCAGT
TTGGGGGTAATGAAGCATTTCAGTTTCGTGAGTAGAGGGCCTACTTTTCTTTCCTCACCCTGTATTTTA
AAAGTCTTCTAAATATGTAGTTTCGGTGTATAATTCTGGGCGGGGGGAGTTTTTTTGGGGGGGGAGCA
AAATATGTTTTGAGTTCTTTTCCCTTTAGGTCTGTCTAGAATTCCAAAGGCAAATGACTCGAGGTAACA
GGCGAAAAGAAAATCCAATATCAGGATAATCAGAACACTGCAGGTTTACAGTTATAGAAACTCTAGC
ACAAGCAGCTTTCACTTTGANGTCTGTGGCGCAACTTGTCCATTGGAAGTAATGGCTGGTAAGTTTATT
CTTTTTTTCCCCACCCCTTTATTAAAATATTTTTATAAAGGACCGTAACTGGGTATTTTTACTTAAATAN
TTTTGGGGGGGGNGNGAACANGGAAAAANGGGGGGGGGAAAAACTTTTTTTTTTTTAAAAATTTTCCC
CCANAATTCTTTTNNNNCTATTGGNCAATTTTGAGTTAAAAAAAAGGAATGGNCTTGTTACCTGGTTN
GCACCAACANCCTTTANGGGATNGGACCANTTTTTTTCNTTGGGAAAAAATNTTTCCCANNNGAAACC
TGNAAAGGGCCNCNCCCCAGGGCNNCTTTTGGGTNATTTTTCNNNCNTTTTNAGGGAAAAACTTNN
TNNAACAAAAGCTGGAGCTCCCCGCGGTGGCGGCCGCTCTAGAAGTAGTGGATCGCCCGGGCTGGAG
GAATTCGNTATGAAGCTTATCNATACCGTCCACCTCTANGGGGGGCCCGGTACCCANTGNNGCCTATA
NTGANNCACATTACAGCCCGGAGGAATGGTAGATGGCANGTGTTCTTGTTTTTAACCAATATGAGGGA
TGTCCTGAATTTGGTAGAACAACTGTGAGACAACTTCAGCAAGCTTTGTAAACAGGTTTTATATTCAGT
TTGGGGGTAATGAAGCATTTCAGTTTCGTGAGTAGAGGGCGTACTTTTCTTTCCTCACCCTGTATTTTA
AAAGTCTTCTAAATATGTAGTTTCGGTGTATAATTCTGGGCGGGGGGAGTTTTTTTGGGGGGGGAGCA
AAATATGTTGAGTTCTTTTCCCTTTAGGTCTGTCTAGAATTCCAAAGGCAAATGACTCGAGGTAACA
GGCGAAAAGAAAATCCAATATCAGGATAATCAGAACACTGCAGGTTTACAGTTATAGAAACTCTAGC
ACAAGCAGCTTTCACTTTGANGTCTGTGGCGGAACTTGTCCATTGGAAGTAATGGCTGGTAAGTTTATT
CTTTTTTTCCCCACCCCTTTATTAAAATATTTTTATAAAGGACCGTAACTGGGTATTTTTACTTAAATAN
TTTTGGGGGGGGNGNGAACANGGAAAAANGGGGGGGGGAAAAACTTTTTTTTTTTTAAAAATTTTCCC
CCANAATTCTTTTNNNNCTATTGGNCAATTTTGAGTTAAAAAAAAGGAATGGNCTTGTTACCTGGTTN
GCACCAACANCCTTTANGGGATNGGACCANTTTTTTTCNTTGGGAAAAAATNTTTCCCANNNGAAACG
TGNAAAGGGCCNCNCCCCAGGGCNNCTTTTGGGTNATTTTTCNNNCNTTTTNAGGGAAAAACTTNN
TTNNNNAAAAGCTGGAGCTCCACCGCGGTGGCGGCCGCTCTAGAACTAGTGGATCCCCCGGGCTGCAG
GAATTNGANNTGANGCTTATCGATACCNTCCACCTCNANGGGGNGCCCGGTNCCCAATNCNNCCTATA
NNGANNCNNATTACAGCCCGNAGGAATGGTAGCATGGCANGTGTTCTTGTTTTTAAGCAATATGAGGG
ATGCCCTGAATTTGGTAGAACAAGTGTGAGACAAGCTTCAGCAAGCTTTGTAAACAGGTTTTATATTCA
GTTTGGGGGTAATGAAGCATTTCAGTTTCGTGAGTAGAGGGCCTACTTTCTTTCCTCACCCTGTATTTT
AAAAGTCTTCTAAATATGTAAGTTTCGGTGTATAATTTCTGGGCGGGGGGAGTTTTTTTGGGGGGGGAG
CAAAATATGTTTTGAGTTCTTTTCCCTTTAGGTCTGNCTAGAATNCCAAAGGCAAATGACTCGAGGTAA
CAGGCGAAAAGAAAATCCAATNTCAGGATAATCANAACACTGCAGGTTTACAGTTATAGAAACTCTA
GCACAAGCAGCTTTCACTTTGAGGTCTGTGGCGCANCTGTCCATTGGAGNAATGGCTGGNAGTTTATT
NTTTTTTTCCCCACCCCTTTATTAAATATTTTTTATAAAGGACGTAACCGGGTTAATTTCAACTTAAATA
TTTNGGGGGGGGGGGNAACAGGGAAAAAGGGTGGGGGAAAAAACTTTTTTTTTTTTAAAAATTTCCCC
CAGAATTTTTTAGGNCTTTTTGGCCAATCNCGATTTAAAAAAAGGAATGGAAATGGCTAACCTGGTNG
GGAACCAACCNCCTTTAGGGANTGGAAAACCCTTTTTTTTNTTGGGAAAAGGTTTTCCCAAAAGGAGN
TCNAAAGGAACAAACCCCCNGCCGGGNTTGGGGNNGCTTTTAAACCCTTCCCCAAGNAAAACCCTCNN
AAATTGGT
TTGANAAANCTGGAGCTCCACCGCGGTGGCGGCCGCTCTAGAACTAGTGGATCCCCCGGGGTGCAGGA
ATTCGGCACGAGGCTTATNTNAANCGTTNGANNGNAGGTGGCNCCCNNTAAAATNTNCTTCANATATT
GNANCNTNTTNNGNATNCCTCATNNATGCNTGGTTGATTGAANANGNGNNATACTAATTAATNTGGAG
NGGNAGGGNNTNCANANGAGNTNCTNTATCATCAGCAAGCCNATAANNTANGATCAATNTATNNTCN
TATGTNGCNGCNTTGCTGATGGTATTCANAATNAAANNCNNGCACNATGCNANCGTGGNTTGNANNC
NTGACTAATGAAANACAGATGCACANCGNGACGNTATGAATACTGCTTNTCNGGAGGATAACGGAAN
GNACGNTGGATAATAANGNTTAAGAAGGCNNNTCNAGATGATNTTGNGGANTTTGNTTTGGCTCTNTN
GAANACTGCTCCCCAGNTAGATNNCCNATNGNTACGTGNTGACTTGANTGGCCTTCAGACTGACGNGG
ACNCTGTGANNGATNNTNTNCCATATGNAACTNTCAGAACTANCANGANAAATTATCCNGGGTCTCTC
TANAATNACTGANTAGAGGANCTNGATNNAAGACNTTTGCTCANNCNCATTTTGGTTTATTATGNTAA
AGGATTTTGCTTGCTCTTTCTTATGNNTGACCGNATATGANGGACTTCANTGTATAATGGAAAAACTTG
NTTGATTTTGACCCCAGGGCCTTATATTNAAGCCNGNGGGAATCAAAAAANTNNNGGTTCNAAATTTT
AAATGGGGTTCNCCTTACAANTTTTTTCCNCTTGGGACTTTTTNCCCATTTCCCGNAAAAGNGTTTTNN
NGNAAANNATTTCCCNNNNTTTNGNNNGGCTTTNCCTTTTNAACNANNNNNNCTGGGN
TNNANCNNNATCTGGAGCTCCACCGCGGTGGCGGCCGCTCTAGAACTAGTGGATCCCCCGGGCTGCAG
GCCTTGGTGTCCGAGCACTCTCTCCCTCTCTCTAGGGCGGCGACCTCCGGCGGCCGGAAAGTCACGAT
GTCCATCCTGAAGGTCCACGCCAGAGAGATCTTTGACTCTCGTGGGAATCCCACCGTTGAGGTTGATCT
CTTCACCGCGAAAGGTCTCTTCAGAGCTGCTGTGCCCAGTGGCGCTTCAACTGGAATCTATGAGGCCCT
GGAGCTCCGGGACAATGATAAGACGCGCTACATGGGGAAAGGTGTCTCAAAGGCTGTTGAGCACATC
AATAAAACTATTGCGCCTGCCCTGGTTAGCAAGAAGCTGAACGTCGTGGAGCAGGAGAAGATCGACA
AGCTGATGATAGAGATGGATGGCACAGAAAATAAGTCCAAGTTTGGTGCGAACGCCATCCTGGGCGT
GTCCCTGGCTGTCTGCAAGGCTGGTGCTGTGGAGAAGGGGGTGCCCCTCTACCGCCACATCGCCGACT
TGGCTGGCAATGCTGAGGTCATCCTGCCAGTTCCGGCTTTCAATGTCATCAACGGTGGCTCTCATGCTG
GCAACAAGCTGGCCATGCAGGAGTTTATGATCGTTCCTGTTGGGGCCCGAAAACTTCCGGGAGGCCAT
GCGCATCGGANCCAGAAGGTTTACCACAACCTGAAGAATGTGATCAAAGGAAAAAATATGGGGAAGG
ATGCCACCAACCGTGGGAGATGAAGGGCGGCTTTTGCCCCCAACATTCCTGGANGAACAAANAANCC
CTTGGAGCTGCTTGAANAATGCCNNTCGGCAANGGCTGGCTACCANCNGACAAAGGCCTTCATTCGGG
CATTGGACGTAACTTGCCCTNTGAGTTNTANAGGTCCGGGCAAGTNTTGAACCTGGACTTTAAAGTCC
CCCGATGACCCCACNNGGTTCATTCACAACCCGNAGAAGCTGGGCCCACCCTGTNCAATTCCTTNN
NNTATCTGGAGCTCCACCGCNGTGGCGGCCGCTCTAGAACTAGTGGGATCCCCCGGGCTGCAGGAATT
CCGCCCGGAGGGCAGTGGACTGCNNTCGCCACCTTCCTCTATGCGGAGGTCTTCGCTGNGCTGCTGCTC
TGCNTTCCCTTCATTTCTCCCAAAAAATGGCAAANNATTTTNAAGTCCCNCCTTGTGGAGTCGGTATGG
ACATATGGCACCACCTTCTTTGGGGCTCTCATTGNCANCCTTGNGCTACAGNACATTGATGCTGTTCGN
GAGATTNAAANNTATGATGATGNGACANANAAGGNGAACCTNCANAACNNCCCTGGGGCTGNGGAG
CACTTCCACATNAAGCTTTNCCGTGCCCAGAGGAACCTNTACATTGNTGGNTTTNCCTTGCTGCTGCCC
TTTCTGNNTANACGCCTGGNGACTCTCATCNCCCACCAGGNCNCGCTGTTGGGCTGCAANAAANNNTT
TAAAAAGCCGGCANAGAGCGCCAATGNTGCNTCCAAGACCTTGNATGGAGGANNAACCACCTCCNCA
AAGANTGNTNCTGGNGGGAACCACCTTNTTGGGGTGGGGACCNCCACTGAAATNGCCGCGGGAATAA
AAAAAGANNCNTNANGNTTTTTTTCAAAAAANCCANAACNNAAAGGNTGGNGTTTTACGTTTNCAAA
GGNGGGAAAANGGNGGGAAAANNNAAAATNTTTGCCNTTTNCCCAACCNNTTTTAAGGGGCTTTNCC
ANGNGGAANTNANACGCCCTGTTTNGGGGGNANAGNNCTAAATTTNGNGAGNNTTNGNTAANTGGGG
CCCCCNNGGCCCANAAAAAAANAAATNANCNTTTCCCCCNCCCNTTNTTGNGNCNNCTTNNCCNNNA
AAAAACCCCCTCTTTTTTTTGAACCCCCCCTATNTNTTTGGGCGNTTTTTTNTCTNANNGGGGGGGGG
GNGGCNCCNCCNTGNCNNTTTTTTTTAAGGGNGACCCGGGTCCCCCCCCCCCNAAAAAAAAANAGGCN
NGGCCC
TCGGCNNNGGGGNGNGCNNCNGNGTCCTNAACTCCGCTNNCNANAGCNANATNTGGAGCNCCACCNC
GGNGGCGGCCGCGTCTAGAACTANTGGANCNCCNCGGGCTGCACGNAATNNGGCNCNAGNNNTTTNN
CCTATTACAGGNCTAACANACACGNNNNACCCNGGGGGNCNCNTGCTGCNCNTNNNACTTNNCNTNC
NTTNNNCNNTAATANANTTCTTNTANNAGATATGATANNANNNNCCNNCGNCNCTCAAACTCNTNTTT
TACCNTNGTTNTNGCNATCCAATCGNNNGCTCGGCGCTGNNGNGNNNATGNNGTAGTTNNNACNNGC
TANAAGNNAGTNANTTNTTNNTNNNTTNNNNTTGTNTNNAGCNGCGNACCGNNTTATATTTNTAATNN
NNACAACGTNGTACTTCCNATTGTNGNNACTCTANNCCTNTNNGTGTAGACCTNTANNNCTNATTCTN
TATNTNNNGNNGNNCNANGNAGTTCATNTNTTTNTTGTNNANNGTNNCNCGNNNANACGNCTTANTTT
ATTNATTTACTNTATGCTGTNGNGNGCTNNNNTNTANTGNNTNNATANTTATNCGACGNNTANNTNNN
TGANTGTNTTANTANTNTNNNANCGTGNNTNATGNNNGNTCTCCNNNNNNNGNNTTCGANGCGNAAG
GNGCCGNNNNCNTNNAACCACTTTANGNAGCNNACGTGTNAACNTNNTCNATGTCNTNNNAGNCCNG
CNNCGNNGNNNNTCGNNTCCNNTNCGCTTGNNNGNNNNTNNANCTATNNTCNTNNNGNTTCTNNNT
NTAATCCTNTTTCTNCATNNATCCGNCCCCCCCCCCTNNCTCNNACNNTCTCNNTATCCNCTGCTCNCN
CANNGNNAACTAGNTNNTANCATTGTATNNCNTGGNATNNTANTTCACCANNGCATGANTTCGCCTNT
NANTCGTNTNATAAATNNNANNACNNCCNATTGNATTACTANATCACNGCTTTNTTAANTGNTCNNCG
NANNNGCNNGCATCTGNGCTTNNCGATNCNCANTTNCNTGTGAGNNGNGANNGACCGTATNNANTNT
NNNNNCNTCTNNCGNNANNNCNGCNCNTACTTNNNCCNCTCTGNTCTCTAGNACNTGACNANTTGNCN
ANCNTCNNNNCCACTCNGNNGANTANACACNGNGNTTCTCTTAATCTCTAATNCGGGACGGAAGGGN
CTCCCNCTACGNGGATNNNGTNGTGANTNTAANCNNNGCTGTNGATANNNTCGCCCCCCCCCCCCCCC
CCCTCTNTTANTTCNACTTNNTGTGTGTNTACNATNAAGCNAANTCTATNCTCTNCTCNCGTGCGTGTG
GGGGGNGGCNNTCNCCC
TTGANNANANANGGAGCTGCNCNCAGGGGCGGCCGCTCTAGAACTAGTGGATCCCCCGGGCTGCANG
NNNGAGNCCNNNTNGTATTTTTATNNANNGNCGGTCNNCNANTTGGGGGGGNATATNGNACNCAAC
ANAATATNNGANNNCGTTNNAAANCNTANCNATGTNTTGTTCTCCAAAGGGGGNGNNCCCNCCTNGC
NAGGGNCNTNNTTGTATTAATAAAAAACGCAGGNAGNTNGGACNNNNTANNCCTANCGNANANANNT
CNTNACTCGGNAACCNTAANNNTGNACAGNTCAGGNGNATTNNTAGNNCTGNNANTNCNTGGNANNA
NNCANANNNTAAANTNCCNNNNGNNNNNNGNNNGTNTTTNNCTCTAATTCNAACNNACCGGNGGNNA
NTTNNNNGTGGNNAGAGNCNNNTGACNCNNGNNTTNCTATNTATNNGNGNTGTGNANANATGNGAGA
CGNGNNCATNGNGANNGGGTNANACNGGTNNAATTNNCNTTTCCNANGACNANNCCTNCCNGCCNAN
GNTTTNGANNAAGCCATANTCCNNNNCACNTACCANCCTTNCTACAANNTNAATACGNCNTGCCANA
ACTNNCNNGGANTNNNNTNNGGCNCNAANCCCNCNANACNANCCCGGNTTANGGTTNNATANGGGCT
CGNGGNACNGNTNTAANTTATAATNATTTACTNAAAGACACCAGTNNNCNATNGACNNGTGNTCACG
NNNAGTNGGNTGANNGTTNNTNANTNCGNCAGATCCNNNTNTTNCGANAANNACTANGAGATNAGGN
GAGTNGCNCCNGGGGNTTCAAANTTTGGATGTTTNGGCNAGACGATNNTNTNTNCNGGNGACANACT
ATNNTACTNGTNGTGNCNNGGANNNGGCACNCTTGTNTANNGTNATNNCTANNAGTAGTGAAGACNN
CGTCACNTAGANTANGATTGGANGNGATNNTNCTCANTATGAGAGTAGACAATGNANNGNGANGANG
NTTNCNGTTTANNTAGNGGNTANTNNNNNCNNNTNTGAGANTNNTNAGNACTCGNNAGCTNTGTANN
NANTGCTTATGCCNCNAAACAANAACNTGGNNCTGAGANTGGNCNTGGNGGNNTCNTNNTGAANTAA
TACGCAGGAAGANCCG
TTGANNANANANGGAGCTGCNCNCAGGGGCGGCCGCTCTAGAACTAGTGGATCCCCCGGGCTGCANG
NNNGAGNCCNTNTNGTATTTTTATNNAANNGNCGGTCNNCNANTTGGGGGGGNATATNGNACNCAAC
ANAATATNNGANNNCGTTNNAAANCNTANCNATGTNTTGTTCCTCCAAAGGGGGNGNNCCCNCCTNGC
NAGGGNCNTNNTTGTATTAATAAAAAACGCAGGNAGNTNGGACNNNNTANNCCTANCGNANANANNT
CNTNACTCGGNAACCNTAANNNTGNACAGNTCAGGNGNATTNNTAGNNCTGNNANTNCNTGGNANNA
NNCANANNNTAAANTNCCNNNNGNNNNNNGNNNGTNTTTNNCTCTAATTCNAACNNACCGGNGGNNA
NTTNNNNGTGGNNAGAGNCNNNTGACNCNNGNNTTNCTATNTANNGNGNTGTGNANANATGNGAGA
CGNGNNCATNGNGANNGGGTNANACNGGTNNAATTNNCNTTTCCNANGACNANNCCTNCCNGCCNAN
GNTTTNGANNAAGCCATANTCCNNCACNTACCANCCTTNCTACAANNTNNAATACGNCNTGCCANA
ACTNNCNNGGANTNNNNTNNGGCNCNAANCCCNCNANACNANCCCGGNTTANGGTTNNATANGGGCT
CGNGGNACNGNTNTAANTTATAATNATTTACTNAAAGACACCAGTNNNCNATNGACNNGTGNTCACG
NNNAGTNGGNTGANNGTTNNTNANTNCGNCAGATCCNNNTNTTNCGANAANNACTANGAGATNAGGN
GAGTNGCNCCNGGGGNTTCAAANTTTGGATGTTTNGGCNAGACGATNNTNTNTNCNGGNGACANACT
ATNNTACTNGTNGTGNCNNGGANNGGCACNCTTGTNTANNGTNATNNCTANNAGTAGTGAAGACNN
CGTCACNTAGANTANGATTGGANGNGATNTNCTCANTATGAGAGTAGACAATGNANNGNCANGANG
NTTNCNGTTTANNTAGNGGNTANTNNNNNCNNNTNTGAGANTNNTNAGNACTCGNNAGCTNTGTANN
NANTGCTTATGCCNCNAAACAANAACNTGGNCTGAGANTGGNCNTGGNGGNNTCNTNNTGAANTAA
TACGCAGGAAGANCCG
TTTNNNNAAAGCTGGAGCTCCACCGCGGTGGCGGCCGCTCTAGAACTAGTGGATCCCCCGGGCTGCAGG
CAGGATCAAGCGATTCCTGGCCAAGAAACAAAAGCAGAATCGTCCCATTCCTCAATGGATTCGAATGA
AAACTGGCAATAAAATCAGGTACAACTCCAAGAGAAGACNTTGGANAANANCCAAGCTGGGTCTATA
AAAANCANGCTGGGTCTATGAAAAGNGGTCTTANCATGTAAACCANTTTTTTAANCAGCCANATCNCA
NNGAAANCNTCNNTACNGTANNGCTNGGCCCANGANGTNNTTTCNTNNNTNTCAGNGNGNAACCCAN
CATTAANTNTAAANCGTNGCAAAAAAAAAAAAAAAAANNTGGGGGGGGGNCCGGGNCCCAATTTCCC
CTTNAGGGGGGNCGTTNAAAAANNNNNNNNNNNNNNNNNNNNNNNNNNNNNNNNNNNNNNNNNNN
NNNNNNNNNCNTTNNNNNNNNNNNNNNNNNNNNNNNNNNNCNCNNNNNNNNNNNNNNNNNNNNNN
NNNNNNNNNNNNNNNNNNNNNNNNNNNNNNNNNNNNNNNNNNNNNNNNNNNNNNNNNNNNNGNN
NNNNCNNNNNNNNNNNNNNNNNNNNGNNNNNNNNNNNNNNNNNNNNNNNNNNNNNNNNNNNNNN
NNNNNNNNCNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNTN
AACAAAATCTGGAGCTCCACCGCGGTGGCGGCCGCTCTAGAACTAGTGGATCCCCCGGGCTGCAGGCA
CGGCAAGTTCAACGGCNCAGTCAAGGCAGAGACGGGAAGCTCGTCATCAATGGAAAGGCCATCACC
ATCTTCCAGGAGCGAGATCCTGCCAACATCAAGTGGGGTGATGCTGGTGCTGAGTATGTGGTGGAGTC
CACTGGGGTCTTCACTACCATGGAGAAGGCTGGGGCTCACTTGAAGGGTGGCGCCAAGAGGGTCATCA
TCTCTGCACCTTCTGCCGATGCCCCCATGTTTGTGATGGGCGTGAACCACGAGAAGTATAACAACACC
CTCAAGATTGTCAGCAATGCCTCCTGCACCACCAACTGCTTGCCCCCCTGCCAAGGTCATCCATGACCA
CTTTGCATCGTGGAGGGACTTTATGACCACTGTCCACGCCATCACTGCCACCCAAAAGACTGTGGATGG
CCCCTNCGGGAAGCTTGTGGCCGTGACGGCCGAAGGGCTTGCCAAAATATCATNCCTGCTTCTACTGG
CGCTTGCCANGCCGNGGCAAGGTCATTCCTTAANCTAACGGGAAACTTACTGGCATGGCCTTCCGCGT
CCCCAATTCCNAACGTGTTTGGTTGNGGATCTGANCCTGCCGCCTGGANAAANCCTGCCTAAGTTTGAT
GANATCAANAAGGNGGTGAAACCAGCGTCAAAAGGCCCTTTNAANGGCANTTTTAGGCTACCCTGGN
GGACCAGGTTGGTTCCTGGGAANTTNAAAAGGGAACTTTANTTTTTNACCNTTNNAAGCTNGGGGTT
GGNATTTGNCCTTAAAANACCCCTTTTGGAAAGCTTATTTTCCTGGGGACNAAAAAATNAAATTTGGC
TTCCCCCAAAAANGGGGGGGGGGGACCTTTTTGGGGCCCAAATGGGCCCCCCAAAGNGAGNAN
NACAAGCTGGAGCTCCACCGCGGTGGCGGCCGCTCTAGAACTAGTGGATCCCCCGGGCTGCAGGTTTT
AAATCAAGTCAACCACTTTTCAAAGTAAAGAGTTCACACAACTACTTTCATGAATTTTTTAATCCCGTT
GGAAACATATACCAGGAGTGTCCAGTATACTGGAATACAGGCACACTACCATTCATGGTATGAATTC
TGGGGTTTACACAACCAGTTTTGATGCAATCTGCTCAGTAATATAATTTGTCATTTTTATTANAAATAT
AATTTCTTCATGTGATGTCATGAAATTGTACATACTGCAGTGTGAATTTTTTTGTTTTGTTTTTTAATCTT
TAGTGTTTACTTCCTGCAGTGACTTTGAATAAATGAAGGAAAAAATTCAAAAAAAAAAAAAAAAAAC
NTGGGGGGGGGNCCNGGCCCCNATTNCCCCTTATNGGGNGNNGNNTNAAAANNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNN
TTNANCAAAGCTGGAGCTCCCCGCGGTGGCGGCCGCTCTAGAACTAGTGGATCCCCGGGGCTGCAGGC
AGGATCAAGCGATTCCTGGCCAAGAAACAAAAGCAGAATCGTCCCATTCCTCAATGGATTCGAATGAA
AACTGGCAATAAAATCAGGTACAACTCCAAGAGAAGACATTGGAGAAGAACCAAGCTGGGTCTATAA
GAAGCAAGCTGGGTCTATGAGAAGTGGTCTTAACATGTAGACCACTTTTTTAAGCAGCCAGATCACAA
TGAAAACATCACTACTGTAATGCTTGGCCCATGATGTTATTTCCTCACTATCAGTCTGAGACCCAGCAA
TAAATATAAAACGTTGCAAAAAAAAAAAAAAAAAACNTGGGGGGGGGNCCGGGNCCCNATTNCCCCNT
NTGGGGNGNNGTTTAACANNNNNNNNNNNNNNGNNNNNNNNNNNNNNNNNNNGNNNNNNNNNNNN
NNNNNNNNNNNNNNNNTNNNNNNNNNNNNNNNNNNNNNNNNNNNNNNNNNNNNNNCCNNNNNNNN
NNNGNNNNTNNNTNNNNNNNNNNNNNANNNNNNNNNNNNNNNTTNNNNNNNNNNNNANNNCCCCC
CNNNNNNNNNNTNCCCCCCCCNNNNNNNNNNCCNTNNNNNANANNNNTNCCNNNNNNNCNNNNNTT
NNTGNNNNNGNNNNNNNNNNNNNNNNNNNNNNNNGNNNNNNNNNNNGNNNNNNNNNNNNNNNNN
NNNNNCNNNNNCCCCCCCCCCCCCCCCCCCCCNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNN
GCCTATNTGGAGCTGCATCNCNGGGGCGGCCGCTCTAGAACTAGTGGATCCCCCGGGCTGCAGGTGAA
TTCCCGTNGANNTTANAGGGAAGCTTTNACAATGNCCGGAGCCCTTGATGTCCTGCNAATGAAGGAGG
AGGATGTCCTCAAATTNCNTGNANCAGGAACCCANTTAGGTGGCACCANCCTTGACTNCCANATGGAA
CAGTACATNTACAAAAGGAAAAGTGATGGCATCTACATNATAAATCTGAAGAGGACGTGGGAGAAGC
GTGTNGGCCGTTCGGNCCATTGCNGCCATTGAAAACCCGGCTGATNTCAGTGTCATATCCTCNAGG
AATACTGGCCAGCGAGCTGTGCTGAAGTTTGCTGCTGCCACTGGAGCCACTCCTATCGNTGGCCGCTTC
ACTCCGGGAACCTTNACTAACCAGATCCAGGCCGNATTCNGGGANCCAAGGCTTNNGGNGGTCAGCG
ATCCCANGGCTGACCACCAGNCCCTCACGGAAGCCTCTTACNTNAACCTGCNAACCATTGCCNTGTGN
AACAGNGACTCTCCTGTGCGCTACNTGGACATTGCCATCCCGTGCANCATCAAGGGAGCGCACTCAGT
GGGTCTGATGTGGTGGATGCTCGCCCNGGAAGTTCTGCGCATGCGTGGCANCATCTCCCGAAAGCACC
CGTGGGGAGGTCATGCCGGACTGTACTTCTACAGAGATCCTGANGAGATTGAAAATGGAAAAGCAGG
CNGCTGNTGANAAGCTGTGANCAANGANGAGTTTTNAAGGGNGANTGGACTGCTCCAGGCTCCAGAA
TTTACGGGNTGGTCNCCTTATGGTGGNGAAACTGNNNTTAAAGCCTTGCAGGTGCCTTCCNNGCCCA
TTTTANCNNNTCCCCATGGAAAACTGGNAGGGCTTAANCCTTCCATTGAAAAACTGGNCTTGCGGCCC
CCCCGGCCCNTGCNAAGGNAATNGGNTNGGAACCNCCCCCNN
NACAAANCTGGAGCTCCCCGCGGNGGCGGCCGCTCTAGAACTAGTGGGATCCCCCGGGCTGCAGGCTT
CGGGGTCGGCAGCGGCAAGACTGTTTGGCCGCCTCTACAAGGTCCCGGCCNTGTTCAGCTCCAGNGCC
AANATNGTCAAGCCCAACGGCNANAAGCCGGACNAGTTCNAGTCGGGGATCTCCCAGGCCNTGCTGG
ANCTGGAAATGAACTCGGACCTCAAGGCGCAGCTGCGGGAGCTGAACATCACGGCCGCCANGGANAT
CNAANTTGGNGGNGGCGGNAAAGCTATTATATCTCGTCCCCGTCGCGCANCTGAAGTNTTTCCAAA
AAATCCANGNGCCCCTGGTGCGCGANCTGGAAAAAAATTTCAGCGGGAAGCACGTCNTCTTCATCCCC
CAGAGGATAATTCTNNCTACGCCNNCTNAAAAAAGCCGTACGAAAAATAAGCAAAANCNTCCCAGGA
GCCNTTCTCTGACCGGTGTGCACGACGCCATGCTGGAGGANTTGTTTCTNCCAAANGAGATCGNGGGC
AAGAAGATCCGANTGAAGCTGGACGGCAGCCCACTNATAAAGGTCCNTCTGGACAAAGCTCAACANA
ACAACGTGGAAGCGCAAGGATGAAACGTTTTTTGGTGTNNANAAACANACTCNCNGGGCANGGGATG
TTTATTTTGAATTCCCCGAGTTTTCAGNTGGNAANCCACACCAACTAAATTTTTATTTTTTTTCTTCNNN
NNNNNNNNNNNNNNNNNNNNNNNNNNNNAAAANANGGGGGGGGGGGGGGGCCCGNNCCCCNNNT
TTTNNTTTTNTNGNGGGGAANNNNTNAAANNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNTNN
NNNNC
NNNNATCTGGAGCTCCACCNCGGGNGGCGGCCGCTCTAGAACTAGTGGATCCCCCGGGCTGCAGGAA
TTCGGCACGAGGTTAGCAAGAAAGCTGAACGTCCGTGGGAACAAGGGAAGAANGAATCCGANCAAG
GCTTGANGANANGAAGANGGATGGGACAAGAAAATAANGTCCNANTTTGGNGNCGAACCCCNTTCTN
GGCCNNNNCCTNGNTNGNTGNNANGGTNGNGNTNGGGAAAANGGGGNGCCCCTTTNCCNNCNCATTG
CCNACTTGGCTNGCNATGNTNANGNCATNCTNCCNNTTCCNGNTTTTAATNGNATNAACNGNGGNTTT
TATNCNNGCNACNANCNNGNCCTNCCNGAATTTANNANCCNTTCTNGTGGGGNCCAAAACNTTCNGG
ANGNCNTNCCCNTTNGAACNAAAGTTTACCACAACCNGAAGAATGTGCATNAAGGAGAAATATGGGA
AGGATGCCANCAACNTGGGAGATGAGAGGCNGCTTTTGCCCCCANCATCTCTGTGAGAANACAANAA
ACCCTGTAGCGTNNGTGAAAAAATNCGCATNTNGNAAAAGCTGTNGTANACACGACACAAGTGCGCC
ATNTNCATNNGACNCANNCTGNCCTCTTGNGNTTNTACACNNGTGGCANNTATTGACACTGTACTNTT
TNGTCGCCCCCNTGACCCCACCCANGTNCATNCTTCACCCCGACGAAGATGNTGCAACCTGTAGAANN
NGCCTTNTCATGGGACCTACNNCACTGGTNGTNTATTNTTAGGACCCCCTTNTAACCNACGANTGAN
CTNGGGAAAGACTTTNTNCAGAAANTTCATCTTGCNCCNCCCGCCAGGGNNATTCCNANGTNGGNTGG
GGGGGGATTGAACCCTCCCCCGGNGGACCCAANCCCCCCCNANCNGGGTTTCNTTCCANANGGNCNG
CNAAAGCANAANAAAAAATCTTTGNNNNANCTTTNCNTCCTTGCTNGGANNAGTTNTNCNCCACAATT
TGGTNTTTNNTNTNANCCNNACGCCCN
TTNAGCNTACTGGAGCTCCACCNCNGNGGCGGCCGCTCTAGAACTAGTGGATCCCCCGGGCTGCAGGA
ATTCGGCACGAGGCTAGAGAGGGGTCCATACGGCGTTGTCTTGAATTCCCGTCGTAACTTAANGGGAA
GCTTTNACAATGNCCGNANCCNTTGNTGTCNTNNAAANGAAGGAGNAGNATGTCNTNAANTTCNTTGC
ANNAGNANCCNNTTAAGGGGNNCCNANCNTNNNNTNCNAANNGNACNATNCNTTTNCCAAAGGNAA
ANGGNNGGGNTTTNNCTTANNAATTNAANAAGNCCNNGGAAAAACNTCTGGTGGGCCGTTCGCCCN
TTGCGCCNTTNAAACCCGGGTNNATGTCATGGCATATATCTGTCAGGAATANNGGGCACACGAGCTG
TGCTGANAATTTGGNGTGNTGCCATGTGAGNNCCACTCTNTATCNGTGGCCGTTTNCNCTCGGAAACN
TTTCTATACGCANANCCGNCCCCNNCNGAGNNCNAGNNTTTGNGGGGCACNCATANCAGNGGTGA
ACACCAANCCCTCNCAGGGNAANCCTTTTTANGNAACCCTCCCCACTTTTGCTGTGGACACAGACTCTT
CTTTTNTGTCTAGGGGAATTTTCATCCNCCGGNAACAACAAAGGGNGCGCNCCCCTCGGGGGCTTCTG
ATGTGGAGGNTTCTCCCCCGANAAACTNCTCTTCCTGCGGNCCACTTCCCAANAAAAACCCCCGGGNG
GACGTTCTGCCCCTCTTTTTTNTTCNAAANNNTTTCCNGAAGNATNANTTAAGGAAGGAACNAGCCC
NCCANCTTNNTAAAGGATTTTTNCCAACCCAAGGAANAANNTNGGGGGGGTGGAAANNGCCTCCCTC
CCCCCCACNANAAATNGANNGGGNTGNCCCCNNNCNGGGGGCCTNACTTTTNAAGGAAGGCCNNG
GNNNTTTTCCTTTCCCAANNCN
TTNAGCNTACTGGAGCTCCACCNCNGNGGCGGCCGCTCTAGAACTAGTGGATCCCCCGGGCTGCAGGA
ATTCGGCACGAGGCTAGAGAGGGGTCCATACGGCGTTGTCTTGAATTCCCGTCGTAACTTAANGGGAA
GCTTTNACAATGNCCGNANCCNTTGNTGTCNTNNAAANGAAGGAGNAGNATGTCNTNAANTTCNTTGC
ANNAGNANCCNNTTAAGGGGNNCCNANCNTNNNNTNCNAANNGNACNATNCNTTTNCCAAAGGNAA
ANGGNNGGGNTTTNNCTTANNAATTTNAANAAGNCCNNGGAAAAACNTCTGGTGGGCCGTTCGCCCN
TTGCGCCNTTNNAAACCCGGGTNNATGTCATGGCATATATCTCTCAGGAATANNGGGCACACGAGCTG
TGCTGANAATTTGGNGTGNTGCCATGTGAGNNCCACTCTNTATCNGTGGCCGTTTNCNCTCGGAAACN
TTTCTATACCCANANCCGNCCCCNTTTNCNGAGNNCNAGNNTTTGNGGGGCACNCATANCAGNGGTGA
ACACCAANCCCTCNCAGGGNAANCCTTTTTANGNAACCCTCCCCACTTTTGCTGTGGACACAGACTCTT
CTTTTNTGTCTAGGGGAATTTTCATCCNCCGGNAACAACAAAGGGNGCGCNCCCCTCGGGGGCTTCTG
ATGTGGAGGNTTCTCCCCCGANAAACTNCTCTTCCTGCGGNCCACTTCCCAANAAAAACCCCCGGGNG
GACGTTCTGCCCCTCTTTTTTNTTCNAAAANNNTTTCCNGAAGNATNANTTAAGGAAGGAACNAGCCC
NCCANCTTNNTAAAGGATTTTTNCCAACCCAAGGAANAANNTNGGGGGGGTGGAAANNGCCTCCCTC
CCCCCCACNANAAATNGANNGGGNTGNCCCCNNNCNGNGGGGGCCTNACTTTTNAAGGAAGGCCNNG
GNNNTTTCCTTTCCCAANNCN
TNNNNAAAAGCTGGAGCTCCACCGCGGGTGGCGGCCGCTCTAGAACTAGTGGATCCCCCGGGCTGCA
GGAATTCGGCACGAGGCTTGCATTCAGTATAACACAAATGTATTCCCAGTCGAAAGTTCAGTAAGGGG
TTGAGAGTAGCAACCAAGAGGTGGAGAGAACAGAGGCTGTTTTTATGATTNGCCAANATATCCAGTCT
CCTNTTTATTNGATTAGNAAAAATATCTTTGNGTNGAATTCAGGGTCAGTTTTGATCCTGTNCTGCANN
AATCTCCANTCAGTAAATNATTTCAGNCANNANCNGAACCNCTNTTANNGAAAANCTANTTACCACCC
TNTCANCACCANAANTCNCCAGGNCNTANCAAAAANCNCAGCNTATTTNGCCGGNNGAAATCTNGCT
GGGCCNTCTTTAAGAATCCNGNCACCACAAANCACTTTTNTCTCNGGGNCTCTTTAANAGATTTATTAT
TAGTATGACCNCNTCTAGGCAGTTGNGTNCNTNCTTNCNCGCCCACTCAGCACACACACGTCCNTCCTT
AAGGAAGCCAANACTTCAGGGAACCTNTNTTCAAAAACAAACTAAGGCTNTNTGATTTACAGGATGA
TGCTGTAAAAGTCACAACGTANCCNTTGGTTGTTGACANCCTGGAGTAGGGGCAAAAATNCCCCNCC
TTTAANGGAGGNCNAAACTTTGGTTTTATNTTGGAAAAATTTNGGGGACAAATTTAGNTTTTTTGGNA
AACAACNTTTTAAAANCCCTGGCCTCACCNTTTGGGGGGGGGTCCCGNTTAANAAGGAGCCCCCCAAT
CTTTTAAACNGGGGTTTTNAAAAACCAGGAGGTTTGGNGGGCCCCAAATTGAAAGGGGGNCCCNCCG
GAAGGGGTTCCCCCTTCCNGCCGGGGGAACCCAAAANCCCCNAAAAGGGGTTNAAAAANGGGTTTTTT
GGGTTNN
TTGGANACNATNTGGAGCTCCACCGCGGTGGCGGCCGCTCTAGAACTAGTGGATCCCCCGGGCTGCAG
GAAAAATTGNAAACNATGNTTCATCGACCTTCCAGCCCCATCAAACATCATCATGATGAAATTTCG
GTTCCCTCCTGGGAATCTGCCTAATCCTACATCCTCACAGGCCTATTCCTAGCAATACACTACACAT
CCGACACAACAACAGCATTCTCCTCTGTTACCCATATCTGCCGAGACGTGAACTACGGCTGAATCATC
CGATACATACACGCAAACGGAGCTTCAATGTTTTTTATCTGCTTATATATGCACGTAGGACGAGGCTTA
TATTACGGGTCTTACACTTTTCTAGAACATGAAATATTGGAGTAATCCTTCTGCTCACAGTAATAGCC
ACAGCATTTATAGGATACGTCCTACCATGAGGACAAATATCATTCTGAGGAGCAACAGTCATCACCAA
CCTCTTATCAGCAATGCCATACATCGGCACAAATTTAGTCGAATGAATCTGAGGCGGATTCTCAGTAN
ACAAAGCAACCCTTACCCGATCTCGNTTTCCATTTTATCCTTCCATTAATNATCATAGCAATNGCCA
TAGTCCACCTACTATTCCTCCACGAAACAGGGTTCCAACAAACCCAACAGGGAATTTNCTTCANACGT
ANACAAAATCCCCNTTCCCCCCCTACTTTNCCCNTTAAGGGACATTNTTANGGGGCCCTTTTTACTAAA
TTCTAAGGTTTTAAAACCTACTAAGGANCTATTCGCACCCCGACCTTCTTNGGGANACCCCANATAACT
TCCCCCCCAGCCAAATCCAATTTAAANAAACCCCCCTTNAAATTAAAACCCGGAGNGGAAACNTTTTT
TNTTTGGGATACCCCAAATTTTTACNAAANAATTCCCCCAACNAAACTTNGGGNGGGNGGTCCTTTNC
CCCTTAACCTTT
TTGAANACAGCTGGAGCTCCACCGCGGGGGCGGCCGCTCTAGAACTAGTGGATCCCCCGGGCTGCAG
GTTTTAAATCAAGTCAACNACTTTTCAAAGTAAAGAGTTCACACAACTACGNTCATGAATTTTTTAATC
CCGTTGGAAACATATACCAGGAGTGTTCCAGTATACTGGAATACAGGCACACTACCATTCATGGTATG
AATNCTGGGGTTTACACAACCAGTTTCGATGCNATCTGCTCGTNCTATACTGGNGNCNTTNTTNTTNGA
ANTATAATTTCNCTNCNTGTGANGNCTTGNNNTTGTNCNTACCGCTTTTGCCGATATTTTCTCCGCTCT
GNNATTTCCNCNNNTTNGNNGNGNNNCNTCNCTNGCAGTGTCCTTTGANTAACTGCAAGGNTCCNTNT
GTNGNCNCNNCGANAACNNAACAANNTTTCGGGGGGGCGNGCCCCCCCCCCCTCNTNTCTTCCCCNCN
CNNGNGGGGNCCGNATTAAAAAATCNCCCCCCCTCCCGCCTNCTNCNCTNTCTTTTTCNNNCTNGCCG
TCGNCTNAGCNCCATGNNGCATTCGNCNNNCGCGTTNCTCCCTNCNTGCNGANGCTACNNTCCGTCNT
NTNTNGTNGGCGNTGNTTNTGTNCNNNTNTCTNCNTNNTCTGANCNTNCNNCTNNACCCCCNCCGCNC
NTCNCCTCCCGCTCCNCNTTCNCCCNNCCNGCCTTTNCCCTCNTNNTNNTTGTCNGTTCGTCATCCTCT
TNCCGNTNCTCTNTNTNCNCCTGCCNCTNTNTNCGACGNATGNCNNCTCGTGCCCNNGNNCGNNGCC
TCCNTCGCNTCNCCNNCNNCTCCTCNNCCGCNNNGCNNGNGCNCNCCNTCTNCNACATGNTGTCGTCN
NNGNTCCTNTTGCGCNCTCNCGNNCGCTNCGGCNGGGGCGCTGTCGCTCGCGCTGCCGACTGCGNACC
TCNTNCNCNNNTCCANCNGCNGNANCNCTCTTTTTTGTCCGNNTCNGCGCGNNCTCGNTNTTCGTAGC
NNNCNAACNGCGTGTCGGCCGTANNTNTTGNCNGCNNCGTCGNCTNNCTNNGGCCC
ANCNCNACTGGAGCTCCACCGGGGTGGCGGCCGCTCTAGAACTAGTGGATCCCCCGGGCTGCAGGAAT
TCGGCACGAGGGTGAGANTNNNAGATGNGNGANCTTGGNANCAACNTTTNANGCTCCCGTTCATTCTG
NATCCTNAANANATGGANNTAACTGNCCTACCTTANCANGCNTGANAGTNTCTGGANNNTGCTACAT
GTGGTNCCNCACCCCCNANGNNNNTTAGAANTCTTTAGNCATTTAGNGACTTACTGTTATAGGGCCNNT
ACATNTAAGANAGCNTGTTNNCGCCNANAATATNTATTTGCANTTTTGTCACAACAACAAAAAAGCTC
AGCGGTNCCTTCTTCATGGNTTGGAGTGTGTGGTAGCAATGCATCAAGCTCAGCTCATTTCCAAGATTC
CACATATCTTGAAGGAGATGTATGACGCAGACCTTTTGGAGGAAGAGGTGATCATTAGCTGGTCGGAA
AAGACCTCTAAGAAATATGTCTCAAAAGAGCTTGCCAAAGAGATTCGTGTCAAAGCGGAACCTTTTAT
AAAATGGTTGAAGGAAGCAGAGGAAGAATCTTCCGGTGGTGAAGATGATGATGAAGATGAGAATATT
GAGGTGGTGTATTCAAAGACGCCNCCCCCCCCNAAAGTTGAAAGCTGTAAAGTCTGACAACCAAAG
ATGATGACNTTTGATATTGGATGNCCNTTNAAAGGGGGGGGGGTGCCACCCCAACCTTAAACAANNG
NAATNGCTTGAAAAATTNTTTTTTGCATTCATTTGGGCCCAAAAAGTGCCAACCATGGAAATGGGGCC
AAAAAGCCTTAAAAATNGGGNTTNAAACATTNAATGCCTTACACNTTTTTTTAACTTNAAAAAAATGG
TATTGGCTTGGGGAAAGGGGGCCCTGGTTTTTTTAAAANCCCCAACCAAAGAAAANTTTTTNGGGGGG
GTCCCNTCG
NTCCTTNNCATTTNTTATNNNANNNNTTTNTNNTCNCCCNCCNCNTNTTTTNNTNCCNCNCCCCCNNT
NGNCCANNGNNNNTTTTATTGGTTTTCTATCTTTNAGGCNNTAGGANNGCANACAGTGGCGGCCGCTCT
AGAACTAGTGGATCCCCCGGGCTGCAGGGGTATGATGCGCANNNGTNACTNCCAGGAGGCCGCGGCC
AGTGCTAAGGAGGCAGGTGAAGCGGCCAAAGCCAGGCGCTGTGAGCGCGGCCTGAAGACACTGGAGT
CCCAGCTGGCTGCTGTGAGGAAAGGCAAAAAGATCAGTGAGGATGAGATCCCGCCTCCAGTGGCCTT
GGGCAGGAGGCCCCTGGTCCCCCAGGAAACAACTAACAAGAGCCCTGAAGCAGAACCCCCANCTGCC
CCCTCCGTGGAGCCAGATAACCCCTCCNAGCCTGANACAAGCCTCTTGGGCATCCCTGGAATTTCTGC
CCCACCCGACNTCANACCCGGACCCACGGGCCCTGCTGCTAGGCCCGGCNNATAGAGTACAAAGTGG
CTGCTCTNAATGCCAAGCGTGCTGGAGACCTANACCGTGCCGGAGAGCTTATGAAGGGTTTGTGAAGA
GATTTGCTGCTGTCCTTGGANGCCCTTGGANAANTGGGNCAGCCTTGTGGNCCTNNATNGCTCATGCC
CCCCCNTNNTCCANANNNACNNTGGAAGCCCCTTCTCANANGGCTTGNTCCANAGCCCCCNNNGTAGC
NCCCCCTCCCGATGGCACCCCTNTNGNANNTNGNANCGGANATGCNACCCACAATNNAATTGGGCTCT
TTTNTACAATCNCCCATTCANNGNCCCNGGTANGGCCCCCTANNGCTTGATTCACNTCCANGAACAAN
TGCGCTTGTGTAATNCCCCNNTNNCANNCTANNANTGGCTTGGATCCAANANTNCCACGCCGNATNGG
CNANNGTCTCNCCCTANNGCNTCCNTGTNGGCAAATTTGGGNGNNATATATNAGCNNCNATAGAGCCC
CCNNNNANNTNCCACNCNTATNCTCNTNTTTTGGNNCANNTACNTATNTAANTTCAAANANANNGGCC
ATTTTTNCTNNTCCCNCNCNCATGGNNCTGTGAGANCNNNCAAATANTTTGTACTCTNTTGACCGCGG
NGTCTANCCTCNGGTANACTTTCCTNNNNANATTTATTCATCCNCTATNTACACNTTGGNTCTNNTGGG
NAGACACNCANNCNCGTNNGGTCTTATCNTNGANAGNNNNNCNATGTNTGAAATGTGATGNTCTCTN
CTCTTATACANGACTNTCTNNACCTNNCAATATACNCTCTGTCNCCC
AAANCTGGAGCTCCCCGCGGTGGCGGCCGCTCTAGAACTAGTGGATCCCCCGGGCTGCAGGGATTNCG
GCACCGAGGNNCCTGTNTNGNNTTCNCAGANTTGGACTNGGNCNCNGGNNNGGNNNNATGAANANCG
NTATTCANACTTNNNCTGCCNNTAGTCAANNNACTTTATAGGGACCNAACGTATGCGCTAATANAANA
TNCTTTACNAACCATTTAANCTGCTATTNCTTGATTTAAAATGGGNNCGCAAAANCCNGTTTGTAACNT
GTTCTTGTGCCCAGTGGCTNANATGACCAGACTCCCATNCGCCTCCAGNTTATACCTNACCTNCCNNTA
NAAGAGAAGACCNNCNGAACNCNATGATTTTNCTCTTNAGACTNNCGNTCTCNATAAAGANCNNNCN
GATATTANCNTNTTCATTGCNCACAAAACAANCTTTGNAANANACAAGGCTTCCACNCNCCCCNCCAA
ATACTAATGGNGCAGNCCTAACNCTGTGNTTTTGATGTTCNCGNCTACCCNTNAANGAGCGGGTGTNG
ANCTTCTGTTNGCNGGGATCCACCCGGNCATNNAACTGGCGGGTGATTNATTTNNTTTACTGCCNATC
NTTCCAATGCTGGAATCCAAGCTGNNATNCCTNGATTGCNNACAAAATGGTATNNTTGAACTCCGCTC
GTTTGNTTTTTCTTCATTGAANGGNACCCCNTGGCCTTNGTTNGGNTTGCCCCCTTTGAANNTGTCAAN
CCCGNNCNTTTNTNGGTTACCCTTNGGGCAAAGGGGAAANNGGTTTCCTTTNCTTCANTTGTTCANGG
NTNTTTNTANNNNNCNTGGTACNTTGTTNTNTCNAAAAACCCNGNTTNNCNGNNTTTCNTCTTAAAAA
ATAAAAAAAACNNTNNTCTATTNTNTGANGGGNTCNCCANTCCCTTNAATTNTNTANGNAAAACNATA
ANTNTNNANACNTNCCNNNAACTNTCNGTNTNNNNNNNTNAAANTATTNTANANAANATTTNCTNCT
NTTNTTC
CAAAAGCTGGAGCTCCACCGCGGTGGCGGCCGCTCTAGAACTAGTGGATCCCCCGGGCTGCAGGGAA
TTTGAGGATTCCTTCGATCTTCAGGCACTTAAGCTGGTCTCAACNCCTGGTCCCGGGGGGGCAGGGCCC
TCCTTTGNNAAGGGTTTCCNAGAANCTGGANGGAAAGGGGGACCAGNCAGGNCAAATGGGGTNGNNG
GCCNCCNNNCCCANTNAAANANCANCAACCNGTNANCGTGNNNACCAAAGGNNGGNNTTTGGGAAN
NNTTGNNTCCCANNTGNAAANTAACNGCTTCCTTCCCTTNCCACNTTNAAANTCANNAAACNTGNAAG
AAAANCATNTGCNAATTCTTTCNNGGTGGAACNGACCGGCNTGATATATCCNGACACGAATATGCNCN
CTGTGACNTTAAAGTAAGGGCNATATATCTTTGACANGTCCCAAAGCACATGCACACTCTGTNNAAAG
TNNTGGAAAAGANNTTTTCTTTCTACTCTTGTGGAGANAATTACTCNNGAANTTANATNGGGNAAGGG
NCCCTNGCCTTNCTCCACTCTNATTTTCTTATNCCCCTNTCCTTCTGAAATGTGTTAACTCATTTCACGC
NATGGTGGGGAAATGCNCTCTCCACCNTCATTNCTTTGCCCTCCNCANCAAAATCAGNGCCNCTGATN
GTCTTTTTTGGGCTTCCCCCTCCTTANGGGCCTTTTCCTGCNCANGAAGNCCCCACACCCNAACCTTTTT
GCTTTTGTCACTGCTGGGGGTTCAAAATTGATNGGGACCAGATCTTTTGTGCCCNCNCAGGAAATTTTT
TTTTTTTTTTTANAAAAATCCTTTANGGGGGAAAAAATTCCTANTGAAACCTGNGGGGCGTNTTNTTTG
CGGTGGCCAACANNAAAANTNACANNNACNCGCCCCANGGAGCCNTGAAATCTTGGTTCCCTGNAAA
AAAAN
TTGAGCCTATATGGAGNTGTATTACAGGGGCGGCCGCTCTAGAACTAGTGGATCCCCCGGGCTGCAGG
NNTCCATCTGTACNTGAGCCATGATGACCTGGNAGCGTCTGGGGGCCCCGGGAAAGGGGNAGGGTTN
GGCCCCAAACTTGGCTTCCGGGGAAGGACNCGGAAACCCTTATGGGGANAACATTGGNCCATCNTTG
GACTGNTTTTGNCTTTCGANCAAGCACCCCCAAATTCGGTGGNGGCAAAANTTTGGGGNCCCAGGACT
TCCCCCNNGCNTTTCCANGGNTGTGAAAANCATTGCTTGCCCCTNNTGCTGACNGCAAATGAGCANAA
GGGCCGGAATTATCCTNGACNANGTTNCTNNACNTGACTGNGCCACAACCCTCGGGGCATTTTTCCAC
CTGNCCCCNTTANGAGGACACCTATNTTGGAGGTGTATCTTGGAGCACGAAAGNGCAGNTTTCCAANC
CACATGNNCTTTTTCTAANGNCCACTTGNGACACCCTTTTGNANAGCNAAAAAAGTGAAAANGGCACC
CANTCCCNNCCCGTGGTGGTTNTTNCGTNGGGGGGAAGGGGGTNCTATTATTTNAACGNGCCAANGTG
GCTTTTGGCCCACCCCNTNTTATTTNACAANGACTGTCCGCAAATNGGGCCCCCAAGGGGTNGCNTTG
TTTCCACAACTTNACGCCCTTCNNGCCCCCNGNNTNGTTACNCGNNGCCTAACACCCNNAACCCCCCC
GAANAANNANCCCCCCCCCGGGANANNGTNCCAACAATTCTCTTNTGGGCNGGGTTTTTTACCTNNT
TTNTTTCGGAATNNTAACCNNANCCTTTTTACCCCAAGGTTTTNCCNANTTNNAGGGAANCCCTAANG
GAGNAAANAACNTCNNCCTAANGGAAGTTTTTACCCCGGAANNTNGNNTNGGAAACCCCTTNTATTA
GGNTTTTTTTTTNCCCTTTCCAACCAATAAATTAANCTTTTAAAAAAGNGAGTTTACCCCAAAATANNC
TATNTNTGGAANCCCCTTTGGTCCTTTATTTGGANCNCCCNNAAAAACTTTAACCCCTTNNNNNGGATT
TNAATTTAATGGGCCCCNNNTTNN
TTGAGCCTATATGGAGNTGTATTACAGGGGCGGCCGCTCTAGAACTAGTGGATCCCCCGGGCTGCAGG
NNTCCATCTGTACNTGAGCCATGATGACCTGGNAGCGTCTGGGGGCCCCGGGAAAGGGGNAGGGTTN
GGCCCGAAACTTGGCTTCCGGGGAAGGACNCGGAAACCCTTATGGGGANAACATTGGNCCATCNTTG
GACTGNTTTTGNCTTTCGANCAAGCACCCCCAAATTCGGTGGNGGCAAAANTTTGGGGNCCCAGGACT
TCCCCCNNGCNTTTCCANGGNTGTGAAAANCATTGCTTGCCCCTNTGCTGACNGCAAATGAGCANAA
GGGCCGGAATTATCCTNGACNANGTTNCTNNACNTGACTGNGCCACAACCCTCGGGGCATTTTTCCAC
CTGNCCCCNTTANGAGGACACCTATNTTGGAGGTGTATCTTGGAGCACGAAAGNGCAGNTTTCCAANC
CACATGNNCTTTTTCTAANGNCCACTTGNGACACCCTTTTGNANAGCNAAAAAAGTGAAAANGGCACC
CANTCCGNNCCCGTGGTGGTTNTTNCGTNGGGGGGAAGGGGGTNCTATTATTTNAACGNGCCAANGTG
GCTTTTGGCCCACCCCNTNTTATTTNACAANGACTGTCCGCAAATNGGGCCCCCAAGGGGTNGCNTTG
TTTCCACAACTTNACGCCCTTCNNGCCCCCNGNNTNGTTACNCGNNGCCTAACACCCNNAACCCCCCC
GAANAANNANCCCCCCCCCGGGANANNGTNCCAACAATTCTCTTTNTGGGCNGGGTTTTTTACCTNNT
TTNTTTCGGAATNNTAACCNNANCCTTTTTACCCCAAGGTTTTNCCNANTTNNAGGGAANCCCTAANG
GAGNAAANAACNTCNNCCTAANGGAAGTTTTTACCCCGGAANNTNGNNTNGGAAACCCCTTNTATTA
GGNTTTTTTTTTNCCCTTTCCAACCAATAAATTAANCTTTTAAAAAAGNGAGTTTACCCCAAAATANNC
TATNTNTGGAANCCCCTTTGGTCCTTTATTTGGANCNCCCNNAAAAACTTTAACCCCTTNNNNNGGATT
TNAATTTAATGGGCCCCNNNTTNN
ANCNCNATCTGGAGCTCCACCNCNGTGGCGGCCGCTCTAGAACTAGTGGATCCCCCGGGCTGCAGGAA
TTCGGCACGAGGCTTNNNNNNAGGNTANCNCAANGGNTNGNCCCATGGCNAGNGCNNTGTNNNNTCN
ANANTTNNNTNACNGNCNAACNANNAGACNTTGCTNACCNTAACNGNCNCTGNNANTTTNANNCCTA
TCATNATTNGTACTANCACAGNCNTAANGNNGACGCTGACCTTTCNCNGANAANGCNCCTTNAAAGG
NATCCNTNCCNCNNNGGGCCATTTTGGCCTTANATNAGGGNCNATTGNCTTTTGATATATTTAGTTAAG
NCCCATCNTTNCCNGAATTTTTTTCCCNTTATAACAAATAAAGCNTCCCCCCCANNNGGGACCTNGGC
GCCTNTTGNCCCNNAACNGGCTTTAACCCANTAACCCCNNTAAANGGCCCACNGCTGNACNCNTTNGN
CTTNTGGTTTTCCGGATTTNATATNNCCGNGNCCCCANNTNTTTTTANATTAANANGGGANCNANANC
NCATATTTTCANANCNCCCTTTNNGCGGTNGNCNNTATNACNNNNNNGNGGGNNCCCAAAAAAANTT
NTAAANNANAAANGGGAANNNTANTTNTTTTTNNNNCCNNTNCCCNAAAAAAAAAAAANAANAAAA
NNCTNNGGGNGGGGGGCCCANANNACNNNNTNTTNNTTTAANNANGGNNCNTACATNNCNNNTNNN
NNNNNNNNNNNNCNCNNNNNNNNTNNTNNNTNCNCCNNCCNTTTTTNNNNNNNANNNCCCNCCCNTN
CTTNTNCNNNNGTTTTTTTTTTTTTNNNNCCCNNCNNNNNNNTNTNTNTNNNTNNNANNNNCNNNNNN
TTTCNNNAAAANAAAAANNNNNNTNNNCNNNCNAANNTNNNTTNCNNTTTTTNTCNTTNNNNNNNTN
ANNNNNNNCNNNTNTNNCGCNNNNGNNTNGTGNGTCCCN
TTTGAANAACATNTGGAGCTCCACCGCGGTGGCGGCCGCTCTAGAAGTAGTGGATCCCCCGGGCTGCA
GGAATCCANCTGNAGGNTNNNGTTNNTGCCCNAGGNTTGCNACNCATGTCATGTCTATCNTGNNGCTG
ACGGNCTGNNNCCTCCACATGCTNATTCNCATGCTGCNTTTTTCGGCNNCTTTGNACANGGCCTGGAG
AACTNTNNNCGGNAAGGAGNCNCTGAATNTTTGTTACGACCGAACATGGAATNNTGACNNNAANNCN
NGNCNCTGCCTTATCANGAACNNTANCNGNTNGNTGAAAGCAGAACACGNCNTCATGGAGCTTTNCC
TCATCCTGTGCTGNCTGCTCTNTTCATGTACATGTGTACAGTGNTTANACCATGCGGCGAGGAGNGCTA
TNCTATGCCACTGNCTNNTGCNAACTTTGNTCCANGGTGGCAGGTGTTTACCGGAGCGCTNATNTACN
CCATTNATGCNNAANAGATTCTGGCGGACTTGCCCGTNGNGNNGCATTTTTTNCTTACTGCTTNACCCT
GTCCTGGGTGGCCTTTCCCCTCGCCCTGGACANCGGNATAATCTACCCCTTNTGCNGAANCAGAAATG
ANGGCTGCCCTCGCCTGGGTCCCAGCCGACTCCCGGNCCCTTCATCTACCCGNAAGCCCCCACCCTTCC
TNCTTTTNTATTTTAAAACAGAAAATAAAACTGTTTAACCCGCCGGAAAAAAAAAAAAAAAAAANTTN
GGGGGGGGGGCCCGGNCCCCAATTNCCCCTTAATNGGNGGNTNNNAANAAANNNNNNNNNNNNNNN
NNNNNNNNNTNNNNNNNNNNNNNNNNNNNNNNNNNNNNNNGNNNNNNNNNNNNNNNNNNNNNNN
NNNNNTTTNNNNNNNANNNNNNNNNNNNNNNNNNNNNNNNNNNNNNCCNNNNNNNNNNNNNNNNN
NNNC
TTGAACACNGCTGGAGCTCCACCGCGGTGGCGGCCGCTCTAGAACTAGTGGATCCCCCGGGCTGCAGG
TTTTTTTTTTNGANGNNTTCNTTTACTTTAATANCNCTACNTTNACCAGGNTATCACCATGGAANGTAA
ATCCAGGTTAAACNAAAATTCACAAGTCCAACAAAGCNTTCTGNGATNNNCCAAAGTTNGNNTAANG
NNNGAGCAAACCAGTTNGCTCATNAANGACTNCCATNGAAGGGATTTTATTTAGNNGANGATNCTTTG
GGAGAAAATAAACNTNCTGCCCNCNTTAAAAANGGGTTTTTNATTNNCCTTTGCNTTAGCNCTNAAAA
TNCANGTTCCAAAANNNTTTTAAAATTTATTNTAATNTNNCNNNCNCCAAAAATTTANTTNTGCATTTN
NCTAAANAGCTAANGGGGGANCCCCCAAGTAAANGCCNCTNNTCATNGTTNNNTGACTCTAAANATA
AAAACCCTACTTTCCAAAGGGGGGCTTNTGTNTNCNANGCTTTTTTAGNTTTNGGGTTAAAAAANNCN
CNCNCNAAACTNTGCGAGGGNNGGGTNNAAAANGGGGNGNNNTCCNNCNNCNCCNGGTCTTTNAAA
GGGGTTNCCTNTCAAANANCCNNCCCNACCCCTAAAANANNNCTCNTNCNGGCCCCANGNGGGCCGG
NAAAAAAGNTNNCATTTACGNGGGGGGAAAANNGNNNTCCCNNNCCTTTCCNTCAANCAGCNNCNGN
NTTTTNNTGGNNNCCCGGNGGGNNCCCNCTTTTNNGNNNNTNGGCANNNGGNGTCTCTTTTTTTAAAA
NACNAATTNTCANCNNAAANGATTNNCANAAGGGGGGGCCNGGGNCCCGCCCTTNCAGGGTNTNTTT
ANCAAAGGCGCACTGNNTNCTTNTNNNGGTCNGGCCCAGACNCTCCCCNGNNANGNGCCCTNNCTTN
GGGNCCATNTCTCCNCATGCCCNTNGGGCCNNNNNCCGAAACANCCGNCTCTTAAANGGGGGNANCN
NTTTTTNAANCANCCCCGTNCCCCNGTGCC
ANCAAAACTGGAGCTCCCCGCGGTGGCGGCCGCTCTAGAACTAGTGGATCCCCCGGGCTGCAGGTTTG
GCTTTAATATAGGAAGGGCCATGGGAGCCCCTGGCTGAACTGAAGCCTACCCCAAGCCTCCATAACCG
GTCTAGGATGAGGCGCTTCCTCTATTCCAGTGATGGATGTGACCGTCCAGCATCACTGACTATGCCCTA
GAGAACTGTGCTCCCAGCCTCACCAGGGTCTCATGGTAAAACTCTGGATTTAATTTTGAAGAAATCCT
ACCCGCCACCAGCCCTACCCTAACACCCAGTTTCCTTGGAGATATATAGCTGGGTTCTAAGCTTCACCA
GGCAAGACGGGATGGAAATCCTGTCACTGAGTACAGGCTGTGTTGTCTCACCCATACCCGCCATGGGC
TTCAGCTCCATTTCAAGCTGCCCAGGGCATTCCTTCATCCCTGGGGTGGCCAGCAGAGTAAGAGAAGG
GGAGGGGTAGTGGGTGTGATTATCTTAATATGCAGGTGAGAGAGGGGCTAGAGGAAGATGGCCATAG
GACAGCTTGGAGTTTTATCCCANGCCAGCTTGCTGTGGGTCTGGTAAGTTGGGTCATGGCTNCNCTGG
GACTGTGGGGAGGAGAGGGACACAGCTTTGAANGCGGTGANGAACAAGTCATATGTTCCAGCTNCGG
GACGTTGTGCTCAAGGAATTTGAAAACCCTTGCTATACCTAAGTCTGGGTACTACANTTTTTTCCACTN
CCCTTTTGNCGCTGGCTTGCCTTAACCCAGGNCCCAAACCCTTTCTGTTTTTANCCTTTAAAAAGGGAC
CCCNGGNAAGCTTAAANCCAAGGNTTTCCCTTACCCCTTTGGCNCTAAAGGGCCTNTTGCAAGGNCCC
CTTGGGTTGGGGNTAANACCAANGGGCCCGGGTTCCAG
NCCTATCTGGAGCTNCACCNCNGTGGCGGCCGCTCTAGAACTAGTGGATCCCCCGGGCTGCAGGAATT
CGGCACGAGGCTTCTATCTTGACCTGCATAAAACAGCGCTGGGAGGGATGTTTAAAAAAATTCAAATT
GCTAGCTTTATACGGAGGGATTGTCTCAGTGGATCTGGGTCTGGGTTGTGGCCCTGGAATCACCACTTT
TAAAAACAGCATCACAGGTGGTTCAGAGAGCACAGTGGGAGAGGCTGCTCTGAGGGATGGGAAAGTG
CAATTCAGTGTTGCCTGGAGGCAAGTGGACACCTTTTGCCTGAGATTCAGAATGGGTGGTCTCCTGCC
AGATACAATCACTGAGAGTTAGGAGTAACAAGGATGCATTTAGTCCAGAGGATTACCCAAGCTCTATG
AAAATGTTTCTCTAAATTAGAAAGTCCTCGTGCCTTCTCTGAAAAATGAATGTACAGGTATCCTGTTTA
CCAAGAGGTGTACTTAGGATTAAAAAACTAGTTTGAAAGTTGTTTTTCTTTGAAAAAGCAAAAATTTA
GCACTGTGACTTGAACCTCTGAATCTTTTCATGCACAGAAAACCGTAAGCCATAAGTAAAAATGAACA
AAAAGGACAGAGAGGATCATAATTTTGAAAATTAGTGAGAGAGTACCCCTTACCAGGGGCCTTCAAG
GATTAAGGACAGAAAAGATTTTANAAAGAAATGTTTCTTCTTGAGAAANGGAGTCTCACATGCCAGAA
CTCAAATTATAAATGTAGTGGTCNTATGACCANGGGGAAGATGCNTCTTGATTTCCCAAAGTGCTCCA
GTTTAAAATACNTAAAATGCTGGCCTTGGGACTTAANNCCCTTTAAAGGAAGNGAATTNCTANTTTTG
GAGCGGTATANGAAATTTCCTCCCTGGATTCCTCTTCCCTTTCAAAAAAANTGATTTGNTAAAAAAGC
NCNTTTNTTCCTTTTTANAACTTTTTTGNATGGCNTTNAAAAATTAAAAAGNCTTTTNN
ANNNCTGGAGCTCCACCGCGGTGGCGGCCGCTCTAGAACTAGTGGATCCCCCGGGCTGCAGGCTCTTT
TTTCTAGTCTTCTTCCCTGTAGGCAGTTTTAGGTGGAGTCAGGGGACAGGCAGCTTTCTGCTGAGGGAG
ATGACACGTCCGTTTTCCAANACAGAAAGTGGTTGGCAGAAGGTGTAAACCCTGAAGTCCCAGTCCCCG
AAATCTCATTTACCCTCAAGGTCTTCACAGTGATCCAGTGTCCTGAACCATTGCCTGAGCAATAGAATA
ACTGTGCTTTTGAAGTAATGTCTGACTCTGAAGGCAGCACCTGACCGCTCCCCCGCGTGCTGGGCTGGT
CGTGCCGATACTCTGGGCTCCCAGTTGCTGCTTCTCAGATATACCTCTTGCCCGACTGCCGCCTCCTCA
GTCCACCTCATCCACTCAACCATGCCCCACAGACATCTTAGGTAGAGGCAGGAGAAGCCAAGGCGTAT
GCACAGCTGCCCAGTGACTGCGCAAAAGAACTCACACTGGTGGCTGAGGCTGGAAGGAGACCAAGAG
TCNNCTTTTCACCACAGGTCACCAGTGTGATGACAATATTCCAGGCTTGACGTGGGAAGAGACAGCCN
ANCTCAGAGTTAACTAAATGCGTGGGGCTGTATTTTAACAACTGGGCAGTTCTCNGAAGGTGGNTCAC
NACTTTAGAAAAAGGAAAGCGACCCTGATGTTNCCTAATTAACCACCATNCANTAGGGGGANGCCAA
ATACANCCTTTGAATTGGCTTAAACTGGAGGAAGGCANCCCCAGGANTTTTTNTTCTTNGCCCCANCA
CNTTTTTTGATTTCCCANCAAANTNCCTAATTTGAANTTTTTAAACTTCAAAAAGGCCNANGGCCTTTT
NGGCTTTTNNAAAANATGGAAAAAAAAGGGNATTTTTTCCCCCTTTATNGCAANGCGGAAAGGGGCN
ANTAACCCNGAANNGGAAAAAANAAACCTTCNNACCANGCCCCGGGAATTTTCCACCNTTTCT
ANNNCTGGAGCTCCACCGCGGTGGCGGCCGCTCTAGAACTAGTGGATCCCCCGGGCTGCAGGCTCTTT
TTTCTAGTCTTCTTCCCTGTAGGCAGTTTTAGGTGGAGTCAGGGGACAGGCAGCTTTGTGCTGAGGGAC
ATGACACGTCCGTTTCCAANACAGAAAGTGGTTGGCAGAAGGTGTAAACCCTGAAGTCCCAGTCCCCG
AAATCTCATTACCCTCAAGGTCTTCACAGTGATCCAGTGTCCTGAACCATTGCCTGAGCAATAGAATA
ACTGTGCTTTTGAAGTAATGTCTGACTCTGAAGGCAGCACCTGACCGCTCCCCCGCGTGCTGGGCTGGT
CGTGCCGATACTCTGGGCTCCGAGTTGCTGCTTCTCAGATATACCTCTTGCCCGACTGCCGCCTCCTCA
GTCCACCTCATCCACTCAACCATGCCCCACAGACATCTTAGGTAGAGGCAGGAGAAGCCAAGGCGTAT
GCACAGCTGCCCAGTGACTGCGCAAAAGAACTCACACTGGTGGCTGAGGCTGGAAGGACACCAAGAG
TCNNCTTTTGACCACAGGTCACCAGTGTGATGAGAATATTCCAGGCTTGACGTGGGAAGAGACAGCCN
ANCTCAGACTTAACTAAATGCGTGGGGCTGTATTTTAACAACTGGGCAGTTCTCNGAAGGTGGNTCAC
NACTTTAGAAAAAGGAAAGCGACCCTGATGTTNCCTAATTAACCACCATNCANTAGGGGGANGGCAA
ATACANCCTTTGAATTGGCTTAAACTGGAGGAAGGCANCCCCAGGANTTTTTNTTCTTNGCCCCANCA
CNTTTTTTGATTTCGCANCAAANTNCCTAATTTGAANTTTTTAAACTTCAAAAAGGCCNANGGCCTTTT
NGGCTTTTNNAAAANATGGAAAAAAAAGGGNATTTTTTCCCCCTTTATNGCAANGCGGAAAGGGGCN
ANTAACCCNGAANNGGAAAAAANAAACCTTCNNACCANGCCCCGGGAATTTTCCACCNTTTCT
TTTGAGNCCNATANGGGAGNTGTATTACAGTGGCGGCCCGCTCTAGAACTAGTGGATCCCGCGGGCTG
CAGGAANTTCNGCNCGAGGCTNTTNNNGGTGTNCCCANACCNGCCNTTTNCCTTAAACCTCNGGAGGN
CCCGCANNGTTAANAGGGGAAATCATTACCNCCATGAGACAGGGGTNGNNCCGNAGANTCGTNNTGG
GGCGAGNTCCACTTGGACACCTTTGTATGNGGNANACATTTTTTNCANTGNAGGTANACTNTAAACG
GACTTATACTTGNCTCTTNANANAAACTANGGACTTTCCCAAATTNAATACNCNACTNNGNACTCCNA
CAAACTCTATTGANCAGNCAATGNATGANNCTNAACGANTTCATAANAGATGANAGAACCANNGCAC
CCCTTNNGNNGAATCNTTNNTGGTNTNCACAGAAANCTNNCTNATTNNNACTNTCATCTATNGNNAAC
CTNCNAGTGTGCGCTCGGGANTTGTAGTCNNCCCANATACNNNNATNNGTGCTTTCAGGAAAANCCTN
TGNCCNCTNNATCTTNTCCTNCTANATAAANCAACNTCTNGCCCCTCCTTCCTNANAAANATTAATCTT
NATTCACCTGCCCTTAAAANTCTTTNGNCNGATNCCNAGATGCNNTTTGACTTGGTTTTTGTNGAANGA
NANANANNNCTTNANTTCTGGCTNCCACCCGCCCCCNATTGNNTTNNNNAAGNAAANTCCACNTTCAN
TTTATANACCTTCTTGAANCTTGATACCNTCGATCAATNCTNTNTGTGGCGTTGGGGGCAATCNATTNT
CCCTNTTTCCCNTTNNTCTTACAATTTTAACTTTNTNNNNANTCCTCGCGANNCCNCTANANTTTNTTTA
GTGNNNNACTTTACAGGNGNNGGCNCCAATTGTCNCNNTGNAGTNTTCTTNACCTCTGGTCCAANATT
CNANTTGTANCGATNTTTGTANCCAACCNATTTTTTNTCTCCANAANNCTTATCNCATACTTGTAATTNT
TTNCNNTGGATGNCGTCNNNNGTTTTNCACTCTTTAANAAANANTCNNCCNNATAGATNNGTNNCTAG
CGGAANTCNTCCTNTTTNCTTCTNNCAATNNNTATTATAACGTTCTTANTNAACNATNCNCNTTCTANT
ATTCTTTAATGGCGNNTATNAANTTGNNTNTTGGTTCNCCNNGTCNTTCTNTTGNCAGNTNCGNNGGA
CTCNNCCCTCCTNTTCGNNATATNTTTAATCTTAATNTTNNNTTTTGCNAGNNNTGCGTTNTTATNNCA
TCTCCTCTGTNGNNACACNNCCC
TTTGAGNCCNATANGGGAGNTGTATTACAGTGGCGGCCCGCTCTAGAACTAGTGGATCCCCCGGGCTG
CAGGAANTTCNGCNCGAGGCTNTTNNNGGTGTNCCCANACCNGCCNTTTNCCTTAAACCTCNGGAGGN
CCGGCANNGTTAANAGGGGAAATCATTACCNCCATGAGACAGGGGTNGNNGCCNAGANTCGTNNTGG
GGCCAGNTCCACTTGGACACCTTTGTATGNGGNANACACTTTTTTNCANTGNAGGTANACTNTAAACG
GACTTATACTTGNCTCTTNANANAAACTANGGACTTTCCCAAATTNAATACNCNACTNNGNACTCCNA
CAAACTCTATTGANCAGNCAATGNATGANNCTNAACGANTTCATAANAGATGANAGAACCANNGCAC
CCCTTNNGNNGAATCNTTNNTGGTNTNCACAGAAANCTNNCTNATTNNNACTNTGATCTATNGNNAAC
CTNCNAGTGTGCGCTCGGGANTTGTAGTCNNCCCANATACNNNNATNNGTGCTTTGAGGAAAANCCTN
TGNCCNCTNNATCTTNTCCTNCTANATAAANCAACNTCTNGCCCCTCCTTCCTNANAAANATTAATCTT
NATTCACCTGCCCTTAAAANTCTTNGNCNGATNCCNAGATGCNNTTTGACTTGGTTTTTGTNGAANGA
NANANANNNCTTNANTCTGGCTNCCACCCGCCCCCNATTGNNTTNNNNAAGNAAANTCCACNTTCAN
TTTATANACCTTCTTGAANCTTGATACCNTCGATCAATNCTNTTTGTGGCGTTGGGGGCAATCNATTNT
CCCTNTTTCCCNTTNNTCTTACAATTTTAACTTTNTNNNNANTCCTCGCGANNCCNCTANANTTTNTTTA
GTGNNNNACTTTACAGGNGNNGGCNCCAATTGTCNCNNTGNAGTNTTCTTNACCTCTGGTCCAANATT
CNANTTGTANCGATNTTTGTANCCAACCNATTTTNTCTCCANAANNCTTATCNCATACTTGTAATTNT
TTNCNNTGGATGNCGTCNNNNGTTTTNCACTCTTTAANAAANANTCNNCCNNATAGATNNGTNNCTAG
CGGAANTCNTCCTNTTTNCTTCTNNCAATNNNTATTATAACGTTCTTANTNAACNATNCNCNTTCTANT
ATTCTTTAATGGCGNNTATNAANTTGNNTNTTGGTTCNCCNNGTCNTTCTNTTGNCAGNTNCGNNGGA
CTCNNCCCTCCTNTTCCNNATATNTTTAATCTTAATNTTNNNTTTGCNAGNNNTGCGTTNTTATNNGA
TCTCCTCTGTNGNNACACNNCCC
TTTGAGNCCNATANGGGAGNTGTATTTACAGTGGCGGCCCGCTCTAGAACTAGTGGATCCCCCGGGCTG
CAGGAANTTCNGCNCGAGGCTNTTNNNGGTGTNCCCANACCNGCCNTTTNCCTTAAACCTCNGGAGGN
CCCGCANNGTTAANAGGGGAAATCATTACCNCCATGAGACAGGGGTNGNNCCCNAGANTCGTNNTGG
GGCCAGNTCCACTTGGACACCTTTGTATGNGGNANAGACTTTTTTNCANTGNAGGTANACTNTAAACG
GACTTATACTTGNCTCTTNANANAAACTANGGACTTTCCGAAATTNAATACNCNACTNNGNACTCCNA
CAAACTCTATTGANCAGNGAATGNATGANNCTNAACGANTTCATAANAGATGANAGAACCANNGCAC
CCCTTNNGNNGAATCNTTNNTGGTNTNCACAGAAANCTNNCTNATTNNNACTNTCATCTATNGNNAAC
CTNCNAGTGTGCGCTCGGGANTTGTAGTCNNCCCANATACNNNNATNNGTGCTTTCAGGAAAANCCTN
TGNCCNCTNNATCTTNTCCTNCTANATAAANCAACNTCTNGCCCCTCCTTCCTNANAAANATTAATCTT
NATTCACCTGCCCTTAAAANTCTTTNGNCNGATNCCNAGATGCNNTTTGACTTGGTTTTTGTNGAANGA
NANANANNNCTTNANTTCTGGCTNCCACCCGCCCCCNATTGNNTTNNNNAAGNAAANTCCACNTTCAN
TTTATANACCTTCTTGAANCTTGATACCNTCGATCAATNCTNTTTGTGGCGTTGGGGGCAATCNATTNT
CCCTNTTTCCCNTNNTCTTACAATTTTAACTTTNTNNNNANTCCTCGCGANNCCNCTANANTTTNTTTA
GTGNNNNACTTTACAGGNGNNGGCNCCAATTGTCNCNNTGNAGTNTTCTTNACCTCTGGTCCAANATT
CNANTTGTANCGATTTGTANCCAACCNATTTTTNTCTCCANAANNCTTATCNCATACTTGTAATTNT
TTNCNNTGGATGNCGTCNNNNGTTTTNCACTCTTTAANAAANANTCNNCCNNATAGATNNGTNNCTAG
CGGAANTCNTCCTNTTTNCTTCTNNCAATNNNTATTATAACGTTCTTANTNAACNATNCNCNTTCTANT
ATTCTTTAATGGCGNNTATNAANTTGNNTNTTGGTTCNCCNNGTCNTTCTNTTGNCAGNTNCGNNGGA
CTCNNCCCTCCTNTTCCNNATATNTTTAATCTTAATNTTNNNTTTTGCNAGNNNTGCGTTNTTATNNCA
TCTCCTCTGTNGNNACACNNCCC
ANACAGTTGGAGCTCCCCGCGGTGGCGGCCGCTCTAGAACTAGTGGATCCCCCGGGCTGCAGGAATTC
GGCACGAGGATGGTGAAGTCCANGGNCATACTGAGAGTATTGCCATATAAATATTTCACCATAGATTTG
AGGATTGACCTAAGGGTTCTCTGTGGAGCACTCTTTTTCAGTGACTCAACTCTGAACTTCAAATTCTGA
GATCTGATGAAACATATAGTTCTCCAACTTGAAATCTGTTTTCTGTCTACATGGAAAACAAGCTATATC
TACACAAATGGAAGCTTATTAATTAAGTGCACTGGTGTCGTAAACTGGCTCCATTTCAGACAAGTTGA
CTCCTTTCCAAAAGAAAGAAGCTAAACAGAAAACTACCCCTTATGCAGAGATGACCAGCCTCTATCAG
CCCTCGGACATTTGTATGTGTCTGGAGGTTCCTGGGGATCACTAGTTTTTTAATTCAGAAGAAGCAGAA
GACATCAGCAGGGTTCCTCTTACCTTACTAATGAATAAACTGAGGCCCAAAGCACCTGATTACAACTTT
TGGTAACTTTCAAATATGCTGGTAAGTGGANTTTTGAAAAAGAAAAACAAAATCAACATGACTGCAGT
GGCGAGATCCGCAAACTCCATGGGGGTTCAGAATTATTGNACAGATGCCCCAAACTTATCCTTGGGTC
TTAAGTTAGGCAAACTCATGAACAAGATATATGCATGTACATACATTTATCTAACTTGNCAGGCTATA
AGANAGACTGGCAACTTAAATGNATACATCCTTCTATTGGCANCCTTTGGGGTCAGCNTTTCTTCNTTA
AAGGTTTTNTTTAANTTTAANTTTTCCTTTTNAAAAAATNTCNTTTTTACCTTCCCTACTTGGGGGGCCT
TTCTAAATAAACCTTTTTNTTTTCAA
TNNNNCCCNNCTGGAGCTCCACCCGCGGNGGCGGCGGCTCTAGAACTAGNGGATCCCCCGGGCTGNA
GGNTTTTTTTTTNGNNGGGNNCATTNACTTTAANANCNCTACATTTCCCANGTTATCCCCANGGAANGT
AAATTCAGGTTAGACGAGANTTCACANGTCCANCAAAGCNTTCTGNGATATNCGAAAGTTNGTNTAAN
GTCTGACCAANCCAGCTNGCTCATCAANGACTTCCATNGNAGGGATTTTATTTAGTNGANGATNCTTT
GGGANAAAATAANCATNCNGCCCACATTTAAAANGGGTTTTTCATTTNCCTTTGCNTTANCACTTAAA
ATNCATGTTTCAAAATAATTTAAAATTATTCTAATNTANCAGCAGCAAAATATAATTCTGCANTTNCTA
AANAGCTAAAAGGGANCCNCAAGTTAAGTTNCTGTCATGTTTATGATTCTAAAATAAATNCTACTTCA
AAGNGGCTCTGTTACCAGCTTTTTAGTTTTGGTTTAAAAACNCNCNCAGACTTGCAGGGAGGTTTATAA
NGGGGGATTCCATGCNCTGGTCTGGAAAGGGTTCCTCAAGAGCCCANCCNGCCCTAAAACAGTNCTC
AGTCCNCAAGGCCGNCANAAAGGTACCATTAACCTGGGGNAAAATAGGATTCCTACCTTTCCTTCAAA
AGCGTTTGGATNTTCTGGCAGCAACTGGGGNNCNTTTTCCAGGATGGCCTTGGTGNGCTTNTTTTAAAA
NTAAATTTTCACNANAAANAATTTAANAAAGGGGGGNCCGGNCCCCCCTTTTCAAGNGNGNTTTTNAA
AAGGGGCCCTGGTTTNTTTTTGGGGGCNGGGCCCGGNCCCCCCCGGGAANGGGCCCCCACCTTGGGGG
ACCCATTNCCCCCCNTNAACCTTGGGGCCCCCNCGGCCAAAAANAAACCCCTCCNAAAAAAGGGGGG
NCCCCATTTTTCAAAAAATCCCCCAGGTTNTTTGGGG
ANCAACAGCTGGAGCTCCACCGCGGTGGCGGCCGCTCTAGAACTAGTGGATCCCCCGGGCTGCAGGTT
TCTACCCATTGCCTGCGGTCATTTCTTTATCTGAGCTCTTTGGTTTGATGTCACCTTTGAGCCAGAAACA
GAGATACCCCTGATATTTTTTTCTTCATATTTCTCTTTCTTTCCTTAGCCCGTAGGTTTTGTCAGTTTTG
ACAGTCGCTCAGAAGCAGAAGCTGCCAAGAATGCTTTGAATGTAAGTACGAATGATGTTCATCTTCGA
GGGTTTCCGTATGAGGTGAAGGGAAAGTAATCGAGTCTCTTTGGACAGGAAAGCAAATGGAAGGGAT
GTGACTACTTGATCTAAGAGGAACGGACTCTAATTAGCCGCATAAAGAGATTTTGCCTAAAATTGTGG
TTAGATGTGCAGACTATTAAATAACACTTTGCTCGTCTCTAGCCCTTACTGTAATGCTCAAGATAACCT
GATGCTGGGGATTTTATTTTTATTCAGCCCAATCACATCCCATGGTGGGTATGCAGTTTACTTCAGCCG
CCATTATATTCTCAGTGGCTAACCCCAAGTCAAGAATAGATCAAAATCTTGGCTTTCCTTAACACGTGA
NGGTTTTAACTTATGTGGCTTTTAAGGGCTTCCGTGGCTGNCCCNTTGGTGAAGACTCTGGGTTNCCAA
TGCACANGCACGAGCTCCATNCCTAGTCAGGGCTGNCTAAAAATATATTTTTNAAAAGNATGTGACTT
TTAAAAAAAAGGTGGAGGCCTTTATTCTGGATTGGATTTAAAAATTGCATATCTGGNTTCCCCAGTAA
ACATTCCGCANTTGGGGGGGCATAAANAAAGGATTTCCTTTTTCCAAGGCCCGAAAGGGGAAAAAGN
GGAAAGGG
NGNNTNNCNATNCCTATTATTAAAANNNNAGTTTCCNNNCNCCCTTTTTCTNCNATCCTTTNNNCCNTT
NNTNCCCNNCCNTNGGGNGGNTNTTNNTTGGNGACTATCTTGAAGNCCTATNGGNNCGCACACNGTG
GCGGCCGCTCTANAACTAGTGGTCCCCCCGGGCTGCNGGCNATTCNGCCACGAGGCAATGNCTTCAGN
CNCNCNNCNNTCCTTTNTTGTATNTNGGNCAATNATTNTGACNATGGNTCACATNATNCGANATNCAT
NACTNTTGTCCCNTNTAGNNTTTTTTNCNNTTCCCTANNTTNNTNNGTNANTCAATNNGNTNTTCCTNC
NNNGATCNNNTNNGTCCNTTCTTTNNNTNNNNNCANNNTNTNCATTTTCTTTGNTTTTCNNNTTNTNAT
NCNTGGNGNTTANGTACTCCCTAGANGNTTTNNNCNNNGTNGGNNTTNGTTNGNTCTTNNANTCTTTN
GCGNTTNTTNCNATANTATNACNGNNNAGAATCNCNATNAACCTNNTNNACTNNTTNTNNTTCTAANG
ACNANTGCTNNTTCTTTTNNCTNNNTTCNNNTNNGTCNNNTGGTNTTTTGTNNTTNACTTTTGTTNGNN
CTTACAGNNTTNTNTGNNNTGNGNATCGTCTCATNNGTTTANTNNANNTTCNCTTTGTTGGNGTTCNNT
TNTNGTNTCGNANTNTANCNTANATNANCTTNTTNGCCNTATNAANNNAGCTTCANNNCNTTANTNNA
GTNCTGANNNNATAATNCGAAGATCNNTNANCAATTNTTTTNTNCTNTTTCNGACGNNTATTTNTCNC
NNGCNNTTNTGCAANCTTNNTNATNGNCAGACCCCNATTTNTTCNANTNTANGNNCTGTNNNGNAGGA
NTCANTANCTNNGNNNNTGCNTNTAATTANNTTTCTCANNTGNANAATATGCTNTCNTTCTNCNNNAN
ATNNNCNNNTANCNANCNGTTGNCAAACNNTTTCTTTTATNNNCTNTCNTTCGTNNAANNNTNNGATT
CACNNTNATGTNTANCTNNTTTGTGNNGCNNNTANTANTTACTATATCTGGGAAANNATANTNTGNCT
AGTNTNTAGNTANAGCANTTTCNNTNCGTATTATCNTTTCCNNCTCCANNNNTNNTNATNTNATGNGA
ATNCNTNNTATNCGTNCNTGNTNTNTTACTTNATNGTCTATAANANTNNTTNTATNCTTCTNTNACTTA
TATATTNATCTCNNNACTGTGTNANTCTGAGNTGANATCGATAATACTACNACTTTATATNNTTTAGNT
NTANTGNGNCGCAGTGTNTGAGNNANATNTGTGTGTGAATGTTATCTGTATNTTTGTTANACAATANCN
GTATATTAACTTATNTCGNACGTNTACNGTCGCTNTCGNGNACGTNATGAGNNTCGTGANNTNATTNN
CGANATNTTAGATNTGNGANTTNAGANACNTGTNTCGCNTCGCGCGCTNNGATTGANTATNTNTTNTN
CC
AANNGTGGAGCTCCCCGCGGTGGCGGCCGCTCTAGAACTAGTGGGATCCCCCGGGCTGCAAGGGCGN
CCGTTCTGGCCGCACTGCGCTCGCCCTGAGCTCCGGGCTCCTGCTAAGCCAGCGGGGNTGTCGCCTCCN
TCCAGTCGCCATCATGATCATCTACCGGGACCTCATTAGCGATGAGGAGATGTTCTCCGACATCTACAA
GATCCGGGAGGTCGCGGACGGGCTGTGTCTGGAGGTGGAGGGGAAGATGGTCAGTAGGACAGAGGGT
AACATCGATGACTCGCTCATTGGGTGGAAATGCCTCCGGTGAANGCGCCGAGGGCGAAGGNNGCGAA
AGCACAGTAATCANTGGTGTCGATATTGTCATGAACCATTACTTGNANGAAACCNNCTTCAGAAAAGA
NNCCTACAAGAAGTAGATCAAAGATTACATGAAGTCAATCAGAGGGAATCTTGAANAACAGANACCA
GAAAGAGTNCTNCTTTTATGACNGGGGCTGCANAACAAATCAAGCACATTCTTGCTAATTAAAAAAAC
TATCAGTTCTTTATTGGNGAAAACATTGAATNCAGATGGNATGGTTGCTCTGCTGGACTACCGNGAGG
GATGGCCCNACCCCATATATGATTTTCTNTANGGATGGGTTAANAGANGGAAAAATGTAACNAGGNT
GGCTNTTCTTTGGATCNATCCCTGTCANCATAACTGGGTTGGGTNCTGGTTTTCTTCCCNCGNCACCNT
NGAATTAAACAGATGGGACTGGANGNCATCTNNANCTTTTTTNTTTGGNTTGAAACGTTNATTAATTG
GGAGCGGNGNGTTTTTTTTTTTGAAAAAAACNGGGGTTNNGGGGGTGGNTAAAAAATAAAACCGCTTT
TAAACTCNAAAAAAAAAAAAAAAAAAAAANTTNGGGGGGGGGGGCCCCCNNCCCCATTTNNNCTTTNN
NNNANNNNNNNTNNAANNNNNNNNNNNNNNC
ACTNGCAGAAGTGNGNNCCGANGNACGACGTGACACNNNCNACNCTTGCNANCNCCCATCTGGAGGT
GNCACTNACANTGGCAGGCCGCTCCTANNACTAGATGGCTTCCCCCCNGGTCTGGCANGTAAGTNGNT
NANNCNGGTNNTTNNGGTAGAANTCNTNCCATCNGGAGNACCACNNCCCGNGNGNGNNAAGTCTNNG
CNCCNNTTNTGNTTNCNNGNNGNAGNNTNGTTGAGGNGCGTGNNNGCGGCANTANNANNGNNNNNGN
NCGGNGGCCNNNNNAGNNGNTNTGNGNGTTNATNNNGNNAGANCGGGNGGNNAGGNAANNTCANNN
NANNGNCCNTGTNNNGAGCCCNGNGGNATTNTNTCNNCTAGNGCNAGNTNCNGNGTTGACTTGNNNT
TNNTNTNNNNGATNCCCCGNCNCGCGNNCNNNNNNTCTNNNCCCANNGANCTGGNNNNCGNNCACCN
NAATTCNNNGNTNTNGTCGGNNNGNTCNGTGNATNNTGTAGTNTNANTNNNCGGTNTNNNCGGNANG
NCTNNGNNATGNGTGGNTNNNGNTNNGANNGTCCGNNTANCGNNTGGNNCNGNGNGNNGGGCCCNT
NANNNNGNCNANNNNNCNACNNNTNANAANGNNNATCTNGCNANTNNANANNNNNNGCNAANCGA
NANNNTNNTANNNNNGNNTTTNNCAANGGGTNGTNNNTGTNNCGNGCCTTGNGGNNCNNNCCNNTNN
TTATCNNNNNCNCTAANNATCNTGNNNTGTNTNCTANNGNTNGNNGNNGTGCNTGCGNNTNTTGTNCN
NTGTCNNGTNGTACNCAGGCTGNGNGCNAGNNNNCNNNNGCNNCTNNANGATCTGTAGNCANGNCAN
GCNATTNCGNNNANTCATACNTNCATCTTNACTAGCTCGTTNAGNNGNCTNCAAGTCTACATNTGNNT
GGNACAATCCNCTGTNCTANGGNCGAGGCGCTTNACTTACNNAAGTTTNNAGTAGNNTCNANTCANG
AGGACGANTGCNTTACNNACTCNNCGCNCNGCGGNANCTNCANNCNGCANCNAGTNNTGTNNTATTA
NNNGTNTTNGCNAGANTNNGANANCACNGNANTNGNGTTGTNCGCGCNCTCNNACNNCNCTGCCNCA
NCCGANNACGNCGNNNGNCNNGGTGTCCNGGTGANCNNATACNNNAGANANAGNTCNGTNCNGANN
NANNGAATANNTCAGCAGTGCACCANTNGCATGNCGCGCANATANACNCGACTNCGNTCNGTGCGAC
NANTGTNTNATNANAGNNCGATCGCGTNCGCGCCCGCNGANACTCNCTNTGNNNTTNTGNTATCNCGA
NCANATNCANGCNCNCGCGCCC
TTNAGCCNATNNGGAGCNGCANNNCNGTGGCGGCCGCTCTAGAACTAGTGGGATCCCCCNGGCTGGA
NGNANTNNGGCACGAGGCAGAAGTTGTGGGAGTTANCAGAAAAAAAGGTGNCCAGCGCACAAAAGA
TAAAGNGNTCACTATGTGAGCCTTCANANAGNGNTCACTGATCCNTGCTCTAAATGGGCCTGATTCTG
GGGCTCGAGCTGACCAAGCGGATATCACCTANGGNGGGGCTGNTCATTTANGGAAATGAAAAAGTAG
GACAAGNCACTTACAGGCCCTTTAAGCACTCTCACTGGCCAGGGCAGANCCCATGGCCACCCCAAGCC
CAGGGAGTGTGATCCTTCCGCGTGCCTTGGAAGAGAAGCAGAGATATTTGGAAGACGATACTACCAAC
TAACACATAGCTGTATTTCTCTCTCTCTACAAAATTTCTTTTGAAAGTATTTTTTCTTTTTAATTGCGGN
GAAATTTACCTTCAGTNAAATGCAAAGACTTAGTGCACACCTTTGTAGATNTTTATGTGCACCCTANAA
CCCATCAACCANATCAAGACACTGAACGCTTTCACACCCTGGTCACTGCCNCGCCTATGGACACCCGC
AGCCTGCCACAAATAACCACTGGTCTTGACCGTTATCACCAGAAGATATGGTTGGTTTAAAAGCTTGA
TGTAAAAAAATTCTGGGGACTCTTGCTCAACATTGNATCTGGNGAAGTNATCCATGGTGGGGATCAAT
TTATTAAATANCTCNCTTGTAGGGGTCCTGGCATGGNGACTTAAAAACCACNGGTTTTTGGAATCCGTT
CCTACTGGTGGGNGGGAAANTTTTGGGTTTGNNNTCCAANCTTGNTAGTTGGTTNTNNAAATAAAAGG
TNCCTTCCNNACCCNNNNNNNNNNNNNNNNNNNNNNNNAAANNNCNTNGGGGGGGGGGGGGNGGGN
CCNCCAAAANNNCCCCCCATATTNGGGGGG
TTNAGCCNATNNGGAGCNGCANNNCNGTGGCGGCCGGTCTAGAACTAGTGGGATCCCCCNGGCTGGA
NGNANTNNGGCACGAGGCAGAAGTTGTGGGAGTTANCAGAAAAAAAGGTGNCCAGCCCACAAAACA
TAAAGNGNTCACTATGTGAGCCTTCANANAGNGNTCACTGATCCNTGGTCTAAATGGGCCTCATTCTG
GGGCTCGAGCTGACCAAGCGGATATCACCTANGGNGGGGCTGNTCATTTANGGAAATGAAAAAGTAG
GACAAGNCACTTACAGGCCCTTTAAGCACTCTCACTGGCCAGGGCAGANCGCATGGCCACCCCAAGCC
CAGGGAGTGTGATCCTTCCGCGTGCCTTGGAAGAGAAGCAGAGATATTTGGAAGACGATACTACCAAC
TAACACATAGCTGTATTTCTCTCTCTCTACAAAATTTCTTTTGAAAGTATTTTTCTTTTTAATTGCGGN
GAAATTTACCTTCAGTNAAATGCAAAGACTTAGTGCACACCTTGTAGATNTTTATGTGCACCCTANAA
CCCATCAACCANATCAAGACACTGAACGCTTTCACACCCTGGTCACTGCCNCGCCTATGCACACCGGC
AGCCTGCCACAAATAACCACTGGTCTTGACCGTTATCACCAGAAGATATGGTTGGTTTAAAAGCTTCA
TGTAAAAAAATTCTGGGGACTCTTGCTCAACATTGNATCTGGNGAAGTNATCCATGGTGGGGATCAAT
TTATTAAATANCTCNCTTGTAGGGGTCCTGGCATGGNGACTTAAAAACCACNGGTTTTTGGAATCCCTT
CCTACTGGTGGGNGGGAAANTTTTGGGTTTGNNNTCCAANCTTGNTAGTTGGTTNTNNAAATAAAAGG
TNCCTTCCNNACCCNNNNNNNNNNNNNNNNNNNNNNNNAAANNNCNTNGGGGGGGGGGGGGNGGGN
CCNCCAAAANNNCCCCCCATATTNGGGGGG
TTNANCNCTATCTGGAGCTNCACCNCNGTGGCGGCCGCTCTAGAACTAGTGGATCCGCCGGGCTGCAG
GAATTCGGCNCGAGGNCGTTNTCGTATNGCCTNNNNNNGATNNNTGCAGGGNANTNNNNNNTNACCT
NAAGGANAANNCCTGNCTCCGANAGAANCTGCAAGGCGGNCCNTCCGNGGTTNGTCCNCTGTTNATT
TNACTANGAAAACCCCANAACNNGAAANTGCTTNGCCNGCCGGGCGNNTNCNNTTTTTTNGNGCTNTG
AACCCAATAAANNTAAATNTNNGTNNAACNNAAGNTNTGTTCANCNGNTTTAATTCCTNNGTCAAGNC
ANTNCGNTTGANNAAAAANCAGCNTTAAAAATATCNGNGATAANAACAATTTGAGGGCNATTCCTNT
TTNTGNCCAGNNGGGANTGTANTTTNGGNGTNCANNCTTTGTCTNGGGNTAGGGCCGAAACCTTNTGT
NTTNNNCCCCTGGGNTTTCCNANCCTGAAAAAANGAATTNCCGGGGGGNNCCTCNCCCNCNAAAGCC
CNNNTNNTTTTGTNTGTTTNANNGGAAGACCTTTTTCANNTTTTTAAACCCCNCCCNGGGGCCAANCCT
CTTCTCNTNNGGGGCCCANCCCNACNTTGGNATTTTNTTTTGGGNAAAAAAANAANCGCNGGNAANNT
GTGCTTTTTTTNTNTGCCCCGAGATTTNCNTTTNATAAACCNCCTGGGGNTTAAAANNACCCTNGGCNC
CNTNTNTTTTTTTTNANAANNATNAGNGGAAAANGGGGGNNNAAAAAAACACCCAGACCCANTTTTT
TGNATGGGGGGTTTTTTTGGGGGGNNTNTCTCCAAAAAANAAAANATTTTNGGNGCGGCTNTTTTTTA
GAAAACNNCAACCATCTTTTTTTTTTGAAGAAGGGGGGNNAANAANNAGGCCCTNCNNCAANTTTTTT
TTNNNTGGGGGGGNCCCCCCCCCNCNCCCACTATTTTTNTTGGGTT
TTNNNNCNNCTGGAGCTCCCCGCGGTGGCGGCCGCTCTAGAACTAGTGGATCCCCCGGGCTGCAGGAA
TTCGGCACGAGGCTGACGAGTATCTTTCACATGATTAACCAAAAAATGCATTACTGCCTTTGGCACACT
GCAAAGCAATCAAATAATCCATTTATTTGAGAATAAAAACCTAAATTTTAAAAAATACATATATTTTC
AAAACGATTAAGAAATTATCCAGCAGTATTCTAGTCTGATTTATATACATAATACTAACCTGTCTTGAA
TATTCTTTCTGACAATAAGAAAATATGATGATTTGAGTCGTTCAATAACTTCACAGTCTCGCTGTTCAC
GGGCAGATAGTTTTCGTGCAACAGGAACTGGCTAAATTGGGTGAGGGGAGAACAAATTTACCGTTGTG
CCATTTTGAAATGACAACACGTAACAGTACGTTTTTGAGATACCAGTTCCTTCTGTCTTTTAAAATGCT
TTCTTGGTAACTTGGTTCTTTGTCCTCAATCATTCTACAGCTTACCAAATTCTACAGCTTACCACATCCA
GCAAATTCACAGCGTGGCCTTTTTGTGGACTGGCTGGCATAATTGGANTTGGTTTTGATTTTTCCTCAG
CTAGTAACTCCTCAGCTTTTGGAAGTTTTCAGCATTCCTCTCCAGTTGNCTGGTGGTGGNTCTTGGAAC
AGCATNTNCAACCCCACCACCTTCAGATGCAGCCTGAAATAACATGTTCCAAACGTGTACCAACCATA
TCAACTGGCATTATCTAAACAAGGTTANTTTTTGGTANGCGTCAAGGAAAAACCCTCTAATNCCATNT
GGAGNTTAATCTATTAAAAAAAGCCNGGTCAAAAANAAATGGGGACTGGGCTTGTCCACTGGAACCC
TGGACTTGCTTAAAAAAAA
TTNNANCNCTATCTGGAGCTCCACCGCGGTGGCGGCCGCTCTAGAACTAGTGGATCCCCCGGGCTGCA
GGAATCGGCACGAGGNCCGTGTTNTGCNTCNAANNCTGCTACGCTANCNCGGTNAANGCTCAGNAA
NTGGANNTGNTATGGTGAACGCTACTGACNTCTTTTCTGTCTCCCGTTGGCGCNTNTTGATAACTNATT
CTGACCCAGTGACTGGGCCAGNTCAATGCGAACCATTTNNTTTATTTATGCGNNANTGGATGNGNNNCG
GGGNTCAAAGCCTTTATGAGAANNTCTACCGTTCTNGGCTNNANGCNAAGGGAGGAGTAAATTTTNNT
TTTGCAGCTGGACCNGNNNNTCTTGGNCAANCCACGCTCCATCTGGTTTGAAGACTATTNAAATTGGC
NNTTCTTGAAGNTNNGCTCGAAGNAANGGGGATGGNGATACCCAACTTCNGAANGCGGCTTTGGNNT
TGGNAANANAGANNCCCCCCTTNTNNNGAGAAGGCCTNTTTNNAATNCCGGATNAGGCAGAAAAACT
TNATTTGGATGGTTGGGNATGGTTNNNACTTTTGTCCNTTGANCTGGANNTTGGATGACCTTTCTAAGA
ACAAAAGGCNCAGGTTAANTTTNTGCCNAATTTNCATTCCCNTGAAACCNGAAAGGNAACCTTTTAAT
TNTGTGGTGGCCAAANATGCAAAAAANTTNCTTTTNAAANNAATTANAAGGGGGGGANTTTNACCGG
GNGGGGTTTTAAAANTGGTNAGCCAATTTNTTTTTTTTNTTTGGNAAANTAATTTTTCCCCCGGANA
AATTTTTTGNGGGGCGGGGCNNNGGGACCCCTTTNTTTCCNNNAAACNTTTTNNCTTNNAAAANAATT
TTTTTTTTNAAAAGGGGCCTTTTTTTTTCNNTTTTNCCCNCCCAAANANGGGGAAAANAAATTTNTCCC
CTTTTTTTTNGCCNCCCGGGNCANGNCTANCCCCNGGGAAAAANGGGGGGCCGATTNTTTTTANTTNT
CACNNNTTTTTTGT
TTNANAAAAGCTGGAGCTCCCCGCGGTGGCGGCCGCTCTAGAACTAGTGGATCCCCCGGGCTGCAGGA
ATTCGGCACGAGGGCACACTGGCGGAAACGGAGGCTCGCTTCGGAGCCCAGCTGGCGCAGATCCAGG
CGCTGATCAGTGGTATCGAAGCCCAGCTGAGTGACGTGCGAGCTGACACGGAGCGGCAGAACCAGGA
GTACCAACACCTCATGGACATGANGACCCGGCTGGAGCANGANATTGCCACCTACCNAAACCTGGTGG
AGGGCCAGGATGCCTACTTCAACGACCTGTCACTCGCGAAGGCCCTGTGAGTCTGGCGGCCTCCTGGC
TTTCCCGCTCTGCTGCAGAAGGAATTCTCCTGGGTAAGAGGCATGGGAGGGGAGGGACCCTTACCCTT
GCCTCTCCCCCTGACCTGCCAATAAAACTTTATGGCCCAAAGGAAAAAAANNATTNTGNAANNGNTNN
NNANANNNNANNNTNNNNATNNNATNNNNNNNNNNNNNNNNNANANNNNANNAAAAAATNGGGGG
GGGGGGGGCCCCCCCCATNTNCTTNANAGGGGNGGGNNNNAAAAANNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNN
AAANCTGGACTCCACCGCGGTGGCGGCCGCTCTAGAACTAGTGGATCCCCCGGGCTGGAGGAATTCGG
CACGAGGCAAGGACTTCACTGCCACCTTCGGCCCCCTGGACAGCCTGAATACGCGCCTCTTCCTGCAG
GGACCCAAGATTGCAGAGGAGTTTGAGGTGGAGCTGGAGCGAGGCAAGACGCTGCAGATCAAAGCCT
TGGGCATAAGTGACCTCAACCGGGCGGGCCAGAGGCAGGTCTTCTTCGAGCTCAAGGGACAGCTGCGG
TCCATCCTGGTCAAGGACACGCAGGCCATGAAGGAGATGCACTTCCACCCCAAGGCCCTGAAGGACGT
CAAGGGCCAGATTGGGGCACCCATGCCCGGGAAGGTGATAGACATCAAGGTGGGAGCAGGGGCCAAG
GTGACGAAGGGCGAACCCGTGTGTGTGCTGAGCGCCATGAAGATGGAGACTGTAGTGACCTCGCCCGT
GGAGGGCACTGTCCGCAAGGTCCACGTGACTAAGGACATGACGCTGGAGGGTGACGACCTCATCCTG
GAGATCGAGTGACCACGCTGAGACTGGCAGCCTGACGATCCCCGGCCCTGCCTTCAGGACACTGTGCT
GCAGGGCAGGCCCAGGCCGGCCAGTGCCCGAGGCCAGGAAGGCCTTGGCCCTGCAGGTNCTGTCCGC
GGCTTTTTACGGCANGACAGACTGGCTTGTGGCGGTCCATTTCCCCACCTTCCCACCCATTNTGTNCNT
TTCCTTGCGGTCGACAAGTCGCTTCACGTTTTTCATTCTTCCTGGCCAAATAAAAGGTCCCCTTCCTTTG
CTTGGAAAACTTGCAAGTGGGCCCTGGGGCCCCGTGGGGAACCANACCTAGGGGGTGCTACCTCCTGC
AGGGGAANAACCTGAGGGCCCAGGTTGNAAAAACTTTGTTCAATAAAAAATGGGTTTCCCCNCCCTTC
AAAAAAAAAAAAAAAAAAAAAANNTGGGGGGGGGGCCCCGGCCCCCCTTTTCCCTTTTANAANN
GCCNATNNGGAGCTGCATCNCNGTGGCGGCCGCTCTAGAACTAGTGGATCCCCCGGGCTGCAGGGNT
AGTTTTTTAGGGTGATAAATACCACCCAAAACCCAAGCTGCCTGCAGCTCTTCCTCCGTGGTCATCTCT
CCTTCGCTTAGGTAACTGAACCTGCATTTTCCACCTAAACAAAGACATGCCATTTGCTATAGCTGCTCA
GCAGGCTGAGTTTATTTCAGACCCTTCCTCANAGGAACAAAGGCCGGAGACACAGGGCTGCCTGTCAC
TCATTTCTTTTTGTTATTGNNGCATCCCCACCCCTGCCCCCAGCACTCACGCATCAAAAAAAAAAA
AANNGTTTNTNTNTNTNTNGTNTTNNNANTTTTTCNNGGNNCNNGGTTNGCNAANTTTTGNAACCCNG
GGGGANAAATNACCNAAANTTTNGCTTTTTGGANGNCCCGGGCCAACTNCCCCCNCAANGGGAANGN
CCCAAAAAGGGGTTNNTTTTAAANCTTNTTNTTTNGACNCCNNGGGNAGGNATTTGTNNNCAAANCTT
TTTTTATATNNNTTTTTTNGAAAAAAANTTTTTTNNGGGCCCTTTTTTTTACTCCGGGNNGGNNANNCN
NNGGNANNAGGNTTTTNAANAACCGGGGGGGGGNCCTTCTTTTTNTTTTNGAANGGNNGGGGGAAAA
ANCNNACCCCGNTAAANTNGATTTTTTNGGGCCCCTANATTTTCCCCGGCGNGGGNNAACCCNTAGTN
GGANNGGGGNNGATNCCCNGNCCCNATNTTNNNTANCTNNGGGGNNNATAAAANCNNTNCNGGNGN
NAAAGGAAAAAAAATNTTCCCCNTTACCCGGGGGCCCGGGGGNTTTNNTCCTTNNNCAAANGGGNTT
TTNTTTTNNTTNNTTCCNCCCCCCCCNTTANAAAAAAAAAATNTTNNAAGTGGNGGGGGGNNGGNGCC
CANTCTNCTNTNATAAAAAAANAAAAAANCCCCTCCCNNCNNGTGCCCCNGGGGAANNTNGTTTCTCC
CAANCCCCTAAAAAAANAGG
TNCCCNATGAAGNGGNCNCNNNTNACNNANNCGCTNGCTTNAACTNCTTTNANGCCCNCATANNGGA
TNCGCATTACAGTGGTCNGTCCGCNCCTANAACTTGTGCGANNCCCCNNGGNCTGCNANCNNTCNGG
NTGCTCTNGTCATNGAANANTGNCANGNCCTCTGCGCTNNGNNCACTCNNTNNNATNTTACNTTGCGG
GNCTCTTTGTGTCCTNTACGNTNNTNTTNTGANGNGNTCNCCCNTTGAANNTNGNNTCTGGGNTAGGT
NTNGNTNAGNNTTTTNTNNTTTCTCTNGNNCANGCNNCNCNNCNNNAGATATTTNCCCGNNANNNTTA
TNTAGNNAGGCCCNNAATGTCCNTTCTCTNTTATGGANCTTNTNTCCANANGNNTNTACNAAATTNTT
NANNTNTNGNCCNAACNATTCCTNCNAGATNNTNTCNGGNATTTNTCTTGNNNTCTATCNNANNNNTT
GTCNCNTTNCTANNANNACCNCCNCNGNCTCCCTTTNATNCTGGNAGCGCCCACANNAAANNGTGC
ATTTGTTATNNTNCCTNNAGGCACTTTGGNAATCTNTNCGGNTTTNTNTNNNGTACNNNNGATGGTGN
TTCTNCNNAGNTCTNNATNANNNANNANATNGCNNNANCTTGANCCTNNTTTNTGNTTNGCNNATNTA
NAAGNANNNCAATTNTNAATTACATNNNCCTAAAGNNGCCCTNCTTNNCAGCNACCNNTCATNNNTTT
ANCCNCNCGGNCTCCCCGTCTCGTTTCATTNCTTTNTTANNNNTNNCTNNNNTAAATAATNGTTTCTTN
ATTNCTNCNNGCNNTAAGNANCGNCACNTNTCTTNCACCTNNNTTNNCNNCNTNCNCNGNATTCNTNN
NCNNNNCNNTCNNTNCTCNGTNTATNCCCNCNGACAGCAANANCNATTGCATNCTGNNTTCNNCCACG
CGCTCTTAACCNACTACNNCNCNNNNGNNTCTTCTTNTNNNTAGCANNTNNCNANAGNNCANNGCNA
CACCTTTANTTANTTCNGTAANNCNNGNTTNTGCTGNTCANCNNGNGNGNCCTNTGTATCANCNNTG
CGNGCTCNCTNNANCANCCCTNGNCANNAATTNCGTGNANNTACTNAATTCANCTCATCTNNACAGTC
TCNTCTGNTTGTNTNCANNTATNNTNANTGGNGACNTGGGCNCCGNTCNTNCCCACTNATNGNNNGT
GTCNNATCTCNTNTNTACCTNNNCNNTGTCTNCNTTTGTTNCTGCATTNTNAANAACTCTGNNTCGTTA
NCNCNCCNTNCTACTATGCTNTCATCTCTNTGTTTCNNGNNATNATNNCTCTTTCTTNTNCG
TTTGAGNCCTNNAGGGGANGTCGTAATTNCNNTGGAGNACNGCTCTAGAACTANGTGGATCCCCCGG
GCTGCAGGNAATGGNGGCACGAGGCAATATNGTGAGCNTCTACNTTANGACNATGNGTNCNAAGCG
GGNNNATNCTTTANTGTGNTCNACTTTTNCAGATNCNATACNCNNNGAGGGCNTNACTNGTNCCNGAT
TGCTCCTANATNGGNACNTAANCNCGGCNCTACNANACNCNCTTATTCTNAGNNACNCACGNTAGACA
CNGTNGNNTNNNNTANNNNTANGNNNNNACNGACACNCNGTGAGGCTCNTGNNNAGTTNACNNCCNC
TNCTCTTANAANNANTTCTTNNCCTGNACAANNNNCAGNAATCNTTNTNGTTNAAANNNAGTAGCNN
NTCNNATNTTCTNAATCTCTTTCNTCTTGCTNTANGACTNAAAAAANCCNNNAGNTTNAANACNCANA
CAGNNACNANNNGCTTTTNTANAGANNCANNTNCGCGACNTGTANGNTNTGNNAAATNGNCGANA
AATTNCNNGNCGNCANGCACNACTNTTTNNNTTTTTCANTTCCCGGTNAAATNTTTAACNTTTNCNCNC
TCACTNACTCNNTCACTTNNCNANCNACTCNAGTANNGNCAANCGNCNNTCTTGNTNCNCNNACTTAT
ATNTTCNANNNANNAACANTNTNCNTCTACANTNNTNNCTNGTCTGNNATNCATACGCNAGNGNNCA
TCTNNGCNNGTCNANTATNNACCTNTCGNCCCNCTAGANNGTNCNCGATNCNAANCATNANTTNNAT
CGTGGTANTCNNNNATCNNNCAAATTNNNNNNGNTNCCCNTGNCNTCTNNNAGTTCTCNNNTNNATN
NNNACNANGTCNANGTNNCNTTACTACNNGTNNNANAGTTGANANTCNANANGNNNCANCGTNGNCG
TCTCGTANAATGGNCNNNNTAATTCNCNTNACTANGTCNNANGCTCTCCGNGTACNGCNATACATGTA
CANTTNNNTCNNGNNNAGCGGNCCNNTNNTNTACTCTCCTNACTANNNNNANGTTANCNCTNANGTA
CAACNCGNTNGACNTTNNTNNANGTATGAAGANNNNTGTNTTANNANNNCGTGGATNCTCNANGATT
ATNCCTANTCAANTAGNTCTNCNCNNCANGANCCANAATNNNNACNCANCACGTATGTCANNNCNNN
TGTACGANCNAGGNCNNACCCGTNGNNTNTCNCACGTNGCTNTCATNCG
TTTGAGNCCTNNAGGGGANGTCGTAATTNCNNTGGAGNACNGCTCTAGAACTANGTGGATCCCCCGG
GCTGCAGGNAATGGNGGCACGAGGCAATATTNGTGAGCNTCTACNTTANGACNATGNGTNCNAAGCG
GGNNNATNCTTTANTGTGNTCNACTTTTNCAGATCNATACNCNNNCAGGGCNTNACTNGTNCCGAT
TGCTCCTANATNGGNACNTAANCNCGGCNCTACNANACNCNCTTATTCTNAGNNACNCACCNTAGACA
CNGTNGNNTNNNNTANNNNTANGNNNNNACNGACACNCNGTGAGGCTCNTGNNNAGTTNACNNCCNC
TNCTCTTANAANNANTTCTTNCCTGNACAANNNNCAGNAATCNTTNTNGTTNAAANNNAGTAGCNN
NTCNNATNTTCTNAATCTCTTTCNTCTTGCTNTANGACTNAAAAANCCNNNNAGNTTNAANACNCANA
CAGNNACNANNNNGCTTTTNTANAGANNCANNNTNCGCGACNTGTANGNTNTGNNAAATNGNCGANA
AATTNCNNGNCGNCANGCACNACTNTTTNNNTTTTTCANTTCCCGGTNAAATNTTTAACNTTTCNCNC
TCACTNACTCNNTCACTTNNCNANCNACTCNAGTANNGNCAANCGNCNNTCTTGNTNCNCNNACTTAT
ATNTTCNANNNANNAACANTNTNCNTCTACANTNNTNNCTNGTCTGNNATNCATACGCNAGNGNNCA
TCTNNNGCNNGTCNANTATNNACCTNTCGNCCCNCTAGANNGTNCNCGATNCNAANCATNANTTNNAT
CGTGGTANTCNNNNATCNNNCAAATTNNNNNNGNTNCCCNTGNCNTCTNNNAGTTCTCNNNTNNATN
NNNACNANGTCNANGTNNCNTTACTACNNGTNNNANAGTTGANANTCNANANGNNNCANCGTNGNCG
TCTCGTANAATGGNCNNNNTAATTCNCNTNACTANGTCNNANGCTCTCCGNGTACNGCNATACATGTA
CANTTNNNTCNNGNNNAGCGGNCCNNTNNTNTACTCTCCTNACTANNNNNANGTTANCNCTNANGTA
CAACNCGNTNGACNTTNNTNNANGTATGAAGANNNNTGTNTTANNANNNCGTGGATNCTCNANGATT
ATNCCTANTCAANTAGNTCTNCNCNNCANGANCCANAATNNNNACNCANCAGGTATGTCANNNCNNN
TGTACGANCNAGGNCNNACCCGTNGNNTNTCNCACGTNTGCTNTCATNCG
TNNAGCNCNATCNGGAGCTNCACCNCNGTGGCGGCCGCTCTAGAACTAGTGGATCCCCCGGGCTGCA
GGAATCGGCACGAGGTGACTATTCTCAACCAACCATAAAGATATTGGTACCCTTTATCTACTATTTGG
GGCTTGGGCCGGTATAGTAGGAACAGCTNTAAGCCTTCTAATTCGCGCTGAATTAGGCCAACCCGGAN
CTCTGCTCGGAGACNACCAAATCTACAACGTAGTTGTANCCGCNCACNCATTGNAATAATCTTNTTC
ATAGTAATNCCAATCNTAATGGGAGGATNCGGTANCNGACTTGTNCCCCTAATAATNGGGGCTGCCGA
TATAGCATTTCCCCNAATAAATAATATAAGCTTCNGACTCCTCCCTCCNTNATTCNTACTACTCCTNGC
ATCCTCTATAGTTGAAGCTGGGGCAGGAACAGGCTGAACCGTGTNCCCTCCCTTAGCAGGCAACCTAG
CCCATGCNGGAGCTTCAGTANATCTAACGATTTTCTNTTTACACTTAGNAGGAGTTTCCTCANTTTTAG
GAGCCATCANTCTTCTTTACAACANTTATCAACATAAGGCCCCCCGGAGGGNCACAATACCAAACCCC
TTTGTTCGTAAGANCCGGAAANAATTACCGGGCGGANTANAANTACTTTTCGCTCCCGTGGAATNAGC
AGNCCGGGCATCCACAAGTGNTTTTTAACAAGCCCGGGAACCCTAAAANACGACCCTTTTTTCCACCC
CGGGGNNGGANGGAGGAAANCCCTTNTTTTTATTATCAACCCNTTTNTTNTTGAATTNTTTTGGGNCCC
CCCCGGAAGGNNTTTTTTTTTTAAATCCCNNCCCNGGGGGGNNNGNAAAAAATTTTTTTTATTTTGGGG
GNCCCTCCCTTCCTGGGGGAAAAAAAAAAAACCCCTTTCCGGNTTTTTTGGGGAAANAGGGNGGGGG
CCTAAAAAGGGCAAAACCGGGTTTTNT
TTNANCNCNATCTGGAGCTCCACCGCGGTGGCGGCCGCTCTAGAACTAGTGGATCGCCCGGGCTGCAG
GAATTCGNNACGAGGCTNANCNATACCGTNGACNTCNNGGGGGGGCCCGGNACCCAANTNNNCCTAT
AGTGNNNCNTATTNCANGNCCNGNNNACNNNGCTTNNNCNNCAGGGTNTGTCTNNCCANTTGGNNCN
AGANNACNTGNGCATTGCAGCTCAGGACTNNACNGGTTNNGAAGGTNACATNNGNATNATTNACCTT
TAAAATNANTCNTGCNCTCATGGCTNTGTTTNTNTTGGCTNTTGCCCCATNTGATGANCACTNTTANGT
GNNTNACCTTGNACCTGATCANTGCTNCCCNCTNNTNATGCNTTGCTTCNTNACANNTGACAGNACT
TACAACATTNTNTAACTGGNTCNACCCGNNTNANTNANAAAAGAACTNGTGNTAACCATTTTANAAAT
CCANGTCACCNNNCTATNTNAGNGATNNATGTGTATNNGGAAANGATCNNTACATTATCAANTGATN
AGAAGCCCTGTNTNNCAATGNACATTNTACNTNATTNTTTAATATGNNGTACNATAAAATTTTAACAT
TTGGCGGACNTTGACTGCTTTGTTAATGATTTGCNGGGGGGNNNNNGCACNCCTTTTGGNGGGGNNGN
NCGGGGGCCNAANTTNGNCNTTTTNTGGGGGNNCNTNAAANANNNNNNNNNNNNNNNNGGNGGNNN
NCNCNNNAANNNAANNTNNNCCAAANNTNGGGGNAANNCCCCTNCCCNNTTTTGGNNNNNNNNNNN
NNCNNNNNNNTNTNNTTTNNNNNNNNNNNNNNNNNNNNNNCNNNNNTTCCNNNNNTNTTNGGNNNC
NNANNNNNNNNNNNNNNNNNNCNNNNNNNNNNNNNNNNNNNNNNNNNNNNNNNGNNNNNTTNNNN
NNNNNGNNNNNNNNNNNNTNNNTTNNNNNNNC
TTNNNCNCTATCTGGAGCTNCACCNCNGTGGCGGCCGCTCTAGAACTAGTGGATCCCCCGGGCTGCAG
GAATTCGGCACGAGGCNCNNNAATNCNTNTNNTAGNTGTNTGGANNCACANGCCGGNCCTNNATATC
NGTTCACCANACTGTGANTCATNAACACNAAGCTTTCTNCCTAGGTTTTNTAATGGCNATTTCGAATTA
GANNAGNCTGTGGNTTGAACTGCANGACTACAGAGGTTGNGAAAATCACAGTNACNTNANCGCCCNA
GAAGNGCATCCCCCCTCTCNTGNCTCAAGTCTGNCTNGGCTGTTCGCCCNATCGTTGAACACTTTTAAG
GTTCTGNCCTTGCNCCTGGNTCACTGNTACCCCTCNCTTAAATNATTAACCCTCCTGAGAGCTGACAGN
TACTCGTTGATATNTATACCTGGNTTNACCAAATTGAATGAAAANGGANCTTGTGCAAANAATTTAAN
AAACCGNTNTCACNACGTNTTGTAAGAGATCNCTGTAATNGGGAAANGTCCTTTACTTTATCAATTAT
GTAAGGCTTTTCTATATCGAATGGNCCTTNNTNCANATCATTCATATGGNGANCANAAAATTTTAAAA
TTAANGGGACTTGACTGNTTTTGTNAANGATTTCCANNNAAAAAAAAAAAAANCNTTGGGGGGGGGN
GGCCGGNNCCCCCCATTNCCCCNNTNTTNGGGGGNGNCNNNAANNANNNNNNNAGGGGGGGGGGGG
GGGNTTTTNTTNCCCCNNAAAAANGCNTTTNNCCCCCCCCCNCCCCCNNNNCNNTTTTTTNAAAANCN
NNNANNANNCCTTTTTTTNNNTNCNNTTNGGAAAATCCCCCGNNNNNNNANNNNTTTTNNNNTNGGG
GAAANTCNNNNGGGGGGNAAAAANNNNGNTCTTTTNNCNCGTNTNNNANNNNGGGGGGGGGCCCCC
NNNNAAANNNNNGGGGGGGGGGNNCCCCTTCCNTGGNCCCNTC
ANCAANAGCTGGAGCTCCACCGCGGTGGCGGCCGCTCTAGAACTAGTGGATCCCCCGGGCTGCAGGA
ATTCGGCACGAGGGGTGGCTGGAGATGGTAGTGGAGAGGGGAGTGTGGGTTTTGGAGTCGAGCAGAC
AAGTATAAACCCTCATCCTCTTGTTACTCGTAGCTGCTTCCTGTTGTTGTAAAGATTAATGACATCATG
TGAAGCACCAGCACACCATCATCATAACAACAATATCTAACTTTATTTAGCCCTATAGTGAGTCGTATT
ACANNNNNNNNNNNNNNNNNNTNNNNNNNTNNNAANNNNNCNNNNNGNTGGNNNGTNNNNNCNNT
NCNNNNNNNNNGNNNANNNNNNNNCNNTGNNNNNATNNTNNANCNNNNNNNNNNNNNNNNNNNNN
NNNNNNNNNNNNNNNCNNNNNANNCNNNANNNNNNNNNNNNNNNNNNNNTNNNNNTNNNNNNNNNN
NNNNTNNNNNNNNNNNNNNNNNTNNTNNNNNNNNNTNNNNNNNNNNNNNNNNNNNNNNNNNNNNNN
NNNNNNNNNNNNNNNNNNNNNNNNNNNNNNNNNNNNNNNNNNNTNNNNNNNNNNNNNNNNNNNNN
NNNNNNNNNNNNNNNNNNNNNNNNNNNNNNNNNNNNNNNNNNNNNNNNNNNNCNNNCNNNNNNCN
NNNNNNNNNNNNNNNNNNNNNNNNNNNNNNNNNNNNNNNNNNNNNNNNNNNNNNNNNTNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNTNNNNNNNNNNNNNNNNNNNNNNNNNNNNNNNNNNNNNNNNANNNN
NNNNNNNNNNNNNNNNNNNNNNNNNNNNNNNNNANNNNNNNNNNNNNNNNNNNNNNNNNNNNNN
NNNNNNNNNNNNNNNNNNNNNNNNNNNTC
AAAGCTGGAGCTCCACCGCGGTGGCGGCCGCTCTAGAACTAGTGGATCCCCCGGGCTGCAGGAATTCG
GCACGAGGCTTGAAGATCAGCCGCCATCATGAACGACACAGTAACTATCCGGACTAGGAAGTTCATG
ACCAACCGACTGCTTCAGCGGAAACAAATGGTCATCGATGTTCTTCACCCTGGAAAGGCAACAGTACC
TAAAACAGAAATTCGGGAAAAACTGGCCAAAATGTACAAGACCACACCAGATGTCATCTTTGTATTTG
GGTTCAGAACGCATTTTGGTGGTGGCAAGACAACTGGCTTTGGCATGATTTACGATTCCTTGGATTACG
CGAAGAAGAATGAGCCCAAACACAGGCTTGCGAGACATGGCCTATATGAGAAGAAAAAGACCTCACG
AAAACAGCGGAAGGAACGCAAGAACAGAATGAAGAAAGTCAGGGGGACTGCAAAGGCCAACGTTGG
TGGTGGCAAAAAGTGAGCTGGAGATTGGACAACAGAAGGAGTAAAGATTCTGCAGTGACTGTATCTG
TGGTGATTGTGCAGATTTTTCATGAAAGAGAGAATAAACTAAGACCTTTTACAAAAAAAAAAAAAAA
AAACCTNGNGGGGGGGCCNGNTCCCNATTTCCCCTTATAGNGAGNTCGNNNTNANNNNNNNNNNNNN
NNNTNNNNNNNNNNNNNNNNNNNTNNNNNNNNNNNNNNNNNNNNNNNNNNNNNNNNNNNNNNNNN
NNNNNNNNNNNNNNNNNNNNNNNNNTNNTNNNNNNNNNNNNNNNNNNCNNNNNNNNNNNNNNNNT
NNNNNNNNNNNNNNNNNNNNNNNNNNNNNTNNNNNNNNNNNNNNNNNNNNNTNNNNNNNNNNNNN
NNNNNCNNNNNNNNNNNNNNCNCNTNTNNNNNNTNNNNNNNTNNNNNNNCNNNNNNNNNNNNCNN
NNNNNNNNNNNNNNNNNNNNNNNNNTTNNNNNNCNGNNCNNNCN
GCNCGNNNTCCNNNNTACTTTTTATNTANTATTATTATTTNTTCTACANTTNNTACNNCTNNATNTTCA
ANTTNANNTCTACNTNTNNTTTCTTCNGTTGGCGTNCGGTTATNTGGCCTTCNCCTTTGANNCCTATATG
GAGTCGTATTACAGTGGCGGACNNTNTATCAACTAGGGGGGATCCCTCCNGGCTTTCACNATTTCTNT
NTATNNANNNTCNNTTACCACCCATAANCCCNNGNCNNNNTTANNANCTTTCNGGCTNTTGANTCACC
TTTAATTTCATTCCCCNTNAANACTANTTAACTAAGNCCTNNCGCNTTCNCGNGNCTTAANTNCTTNNT
TNTTCNTCCCTTCCNTCNTNCCNANTNTGACACTNTNNTNGNNCCAGNCNTNTCANTNNTANTTCTNT
TATNCACACNCGCNNNNCTNTTGCNNGTNTNANTCTATNTNAGNACNNTCACNNNTNCNTCTNTCTGT
CATTTTCCTNCNNNATNNTACTNTTNTATNNCACTNCACATCTGTATCNNCNNTCTNCNNTTATTTCC
AGACGNTACNATTNNCTCNANTATTTTTGNGCTCCNTNNNTNATNGATCNTATNTTNTTANNTGNCTGA
CNCCCTNAGACNGTNNATATNACTTNATTTNNTTNNNNTTNNCTNNCANTCANNNNNGATACTCGTAT
CNCATTGTNNACCNNTNCTNNNACTGTANNCATTTNNCTANTTNNGNTTTANANNANATNNCTNTNCC
AGATGACCNTTTATTTTCNATTCTCTCTNCATTTCCAGNNTCCNNANATCATTATTAGNCNACTANNAN
TCNTCCATANNAATNCTNTTATTTTACTTGACTCTCTTNANCNGATANTTNCCNTNGTANNTNTCTTTT
ANACCACTCCTNTGNCNCNATNANCTCTTTNAACNTACTNCCCGTCGTCTTCNACNTGCNTCTNCCNTN
GNCCNTATTTNATTTTGCNNTNANCCCNNNTCCTNACTTCNNGTCATACNACTNGACTCGNATCTACCN
NTTCCTATGACTCNNNANNAGGNTATCNTCNTATGTNTNTNTGATNCTTCGTAATTCNNTCGGNTNNCT
NCTNNTNCGTTTNTTTNTTTGTANTGNNNNTTTCNTTCTNCNACGTACGTCCATCNNTNCNGTNNCTTN
NTNGNNNCNATGTNTNNCTNNNGACNNACNAANTACTCNCGTGTCNTNACNCATCGATCTCTCTCACA
TATNCTTACNATCGANAANTTATANTCNNTCTTGTTAGTCANNTATCATNTCTNNNCNNNANNNTNNA
TTNCNCTGTTAGCTNTNNACTAAGCGNGANNNCNTANATANGNTNTATCTCATNAACNCCTANCGTAT
ATTNGCTCTCTCCGTNACTCCAACTCNTCTAATNCGCGCTNNCTCCNCGACANACTTATGCTCTATCCA
TTCCTCACANTAANCATNTGTATCNTATCNANGGNTGCNACGACNANANANNATTGNCTGTNNTNNTC
ATCNACGCACGCTCTNCTNCCTACGNTNGTTNACGNTCTNNATCAANCTCNCCTANCTNTNTTCCNNTC
TCTACTTATGNGATCCTNCNCGATTTANCCNCNCCGTGTGTATGCNAGNCTCTTNNGTGATANATACGN
NCCCTCTNATCCNCTATNCGATNTCCG
CTCGACNGNNGCGGGGTNGGCCNGGTGANCNGCTCNTGACNCCCTNTNCAGCCCTCATAGTGGAGTN
CGTATTACATNNNAGGNCGATCCCTCNGGCTGCGTNTCCCNCANGCCCNNNNCTCCCTNNGGNGNTGC
NNTNNCTATNTTNTTTCNCTNTCNNNNTCTNCACNTTCTCNANNCGCCTTGNCNNNNTGGNGNNNCAT
NCANATNTTTTTNCGCTNTCGTNCNANCGTCGCNCNGTATNGGNCGTAGNGNNNCCGGGGNNGNGGC
CGNTCCANCNCCCGCTTATNNCNTNGGGANNCCACCCNCNCGTNCNNCCNCTATATCCNCNNCCNGTN
CTTTTTGTNCCGNCCCGNNGGNCNGNTGTNNNCTCTNNTNTNCCNCCCNCTGCNGTNNCTNCNTNTCC
NTTTNTTTTNCNCTNNNTNTNCNACGCATGNTNNNCGTCCGCNNGTCCCTCTNNNNNGNNNCTTNTNN
NTNCTNTCNNTNTTTTCTACGTANTTGGTCGAGGNCGNNNNNNCCACTGCNTGCTGTTNCCTNGGNGN
NGANGNGCCNGGCTTCTNTCCTGTNTTNTCNCTCNNNCCTNCGTCTGANCCNNGNTTTNNCNTCNCACT
GTNNGTTTAGNGCCGGCNNGCGCNTGTANNTTNCACNCCTNCCTTATTNNTGCGNGGCNNCNTCGATN
NATTNNCTTTCANNNCTNTGATCCCCACAANCNNNTGNTNTCNCNACNNNCTCTNTCCCNTATCCNCTC
ACTNCTCNNATGGGGNNNTTTCCCTNTTCNCCNNTNTTNTATCTTCNTANCTTTCCCTATNTCCCGTNN
NNCCTCCNCNNATNTNNCCGCGCATNGNTCNCTCGTGNNTTACATCNNNCCNTCTGGTTGCTCCNTCN
GANGATNCNTTAACNCTTNCTNCGTGNTCGNCNNCNTNCTTNCNATNNTNTACNGCCGNCTGNNCGNN
ATTNACTGNGNTCCCGCNGGNCNATTNNTNCNTTTCATCCTTNNNATTTCTTANTNCNTNTACNNNCGT
GGTCGTNGTAATCAGCTANCCCTCAANGTGNCCTCTNTGTCNNTNNNNTNCGNCTGTCTNGGCNNCNT
CATATGNNNTNTNTNTNNCNNTCGANTNCTCNCACNTTCGTNCNTGCTTCCGGGNGTNCNGAGCGACN
TTCGTTGNTCNGTNCCCCCNCTCNGTTCNNTCCTGANGNGNGTGNCNCNNNTGTNTNCTTNCTTTCTCT
NCGTATNTTCTCNNTCGNCACNNCTCGNNGNCGNAGCGANAGNAGTGNNCGTNANANCTAGTCNNNC
TGNGCAACTGANGNACNGCGTNATAGTCNTATGCCCGCGNTNNGNGNACATGTNTCTGNNCNTTNNCN
NATNTGNNNNNGCTNNNANGTNTNACATCTCGCTCGANNTNNTCACTNCGACGCGCGGTCGTCGCTCC
GACNCATNNTCTGTNTNNAANGGNTNNNTCNANCTNNNCACG
GCCTATNNGGAGCTGCACCNCNGTGGCGGCCCGCTCTAGAACTAGTGGATCCCCCGGGCTGNAGGAAT
TCGGCACGAGGCAGAGTTGTGGAGTTACAGAAAAAAGTNGTCCAGCCCACAAAACATAANGTGGTTN
ACTATGTGNACCCTTNAAAAAGGGTCANTGGNNCNNGNCTTNAANGGGCCNNANTNTGGGGNNTGAN
CNNNCCNANCNGANNTNANCNAAGGGGGGGNTGGTNATTTAAGGAAATGAAAAAGNANGACNAGNC
ACTTACNGGCCCTTTAANCNCTNTNACTGGNCNNGGCNNANNCCATGGNCACCCCAAGCCCANGGAG
TGTGANCCTTNCGCGTGCCTTGGAAGAGAANCCNAGATATTTGGAAGACGATACTACCCCTAACACAT
ATGCTGTGTTTTCTCTCTCTACANAATTTTTTTGAAAAGATTTTTTCTTTTTAATTGCGGGNGAAAATTA
CCTTCAGTAAAATGCGNAGACNTAAGTGCNCACCNTGTNNATTTTATGTGCNCCCTATACCCNTCNCC
CAATCAAGACACTGGACGCTTTCACACCCTGGTGACTGTNCCGCTATGCACACNCGCAGCCTGGNACA
AATAACACTGGTGTTGANCGTGTTAACAAGAGATATGTTTGGTNTTAAANCCTCATTGTAAAAAATAC
TGGGGACTCTTGCTCACAATTGTTCTGGGAGGTATTCNTNGTGGGGATCATTAATACCCCCTTGN
GGGGTCTGGCATGGNGACTTAAAACACNGCTTATGAANACNTTCCTACTGGTNGGNGGGAAATTTGGG
GGTGGGNGCCANCTTGTNNGTTGGTTTTNAATAAAGNNCCTCCNAACCNCCNNNNNNNNNNNNNNNN
NNAAAAACCCNGGGGGGGGGGGGGGGGGGCCCNANTNTTTNNCTTTAANNNNGGG
GCCTATNNGGAGCTGCACCNCNGTGGCGGCCCGCTCTAGAACTAGTGGATCCCCGGGGCTGNAGGAAT
TCGGCACGAGGCAGAGTTGTGGAGTTACAGAAAAAAGTNGTCCAGCCCACAAAACATAANGTGGTTN
ACTATGTGNACCGTTNAAAAAGGGTCANTGGNNCNNGNCTTNAANGGGCCNNANTNTGGGGNNTGAN
CNNNCCNANCNGANNTNANCNAAGGGGGGGNTGGTNATTTAAGGAAATGAAAAAGNANGACNAGNC
ACTTACNGGCCCTTTAANCNCTNTNACTGGNCNNGGCNNANNCCATGGNCACCCCAAGCCCANGGAG
TGTGANCCTTNCGCGTGCCTTGGAAGAGAANCCNAGATATTTGGAAGACGATACTACCCCTAACACAT
ATGCTGTGTTTTCTCTCTCTACANAATTTTTTTGAAAAGATTTTTTCTTTTTAATTGCGGGNGAAAATTA
CCTTCAGTAAAATGCGNAGACNTAAGTGCNCACCNTGTNNATTTTATGTGCNCCCTATACCCNTCNCC
CAATCAAGACACTGGACGCTTTCACACCCTGGTGACTGTNCCGCTATGCACACNCGGAGCCTGGNACA
AATAACACTGGTGTTGANCGTGTTAACAAGAGATATGTTTGGTNTTAAANCCTCATTGTAAAAAATAC
TGGGGACTCTTGCTCACAATTGTTCTGGGAGGTATTCNTNGTGGGGATCATTTAATTATACCCCCTTGN
GGGGTCTGGCATGGNGACTTAAAACACNGCTTATGAANACNTTTCCTACTGGTNGGNGGGAAATTTGGG
GGTGGGNGCCANCTTGTNNGTTGGTTTTNAATAAAGNNCCTCCNAACCNCCNNNNNNNNNNNNNNNN
NNAAAAACCCNGGGGGGGGGGGGGGGGGGCCCNANTNTTTNNCTTTAANNNNGGG
TTNNANAAAAGCTGGAGGTCCACCGCGGTGGCGGCCGCTCTAGAACTAGTGGATCCCCCGGGCTGCAG
GCAGGATCAAGGGATTCCTGGCCAAGAAACAAAAGCAGAATCGTCCCATTCCTCAATGGATTCGAATG
AAAACTGGCAATAAAATCAGGTNCAACTCCAAGAGAAGACATTGGAGAANAACCAAGCTGGGTCTAT
AANAAGCANGCTGGGTCTATGANAAGNGGNCTTANCATGTANACCACTTTTTTANGCAGCGAGATCAC
AATGAAANCATCACTACTGTAANGCTNGGCCCATGATGTTATTTCCTCACTATCAGNCTGAGACCCAN
CATTAANTATAAANCGTNGCAAAAAAAAAAAAAAAAACNTGGGGGGGGGCCCGGNCCCCAATTCCCC
CTTATNGGGNGTCGATAANAANNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNNNNNNNNNNNNNNNN
NNNNNNNNNNNNNNNNNNNNNNNNNNNNNNNNNN
ANANCTGGAGCTCCACGGCGGTGGCGGCCCGCTCTAGAACTAGTGGATCCCCGGGCTGCAGGNTTTAA
ATCAAGTCAACCCTTTTCAAAGTAAAGAGTTCACACAACTACTTTCATGAATTTTTTAATCCCGTTGGA
AACATATACCAGGAGTGTTCCAGTATACTGGAATACAGGCACACTACCATTCATGGTATGAATTCTGG
GGTTTACACAACCAGTTTTGATGCAATCTGCTCAGTAATATAATTTGTCATTTTTATTAGAAATATAAT
TTCTTCATGTGATGTCATGAAATTGTACATACTGCAGTGTGAATTTTTTTGTTTTGTTTTTTAATCTTTAG
TGTTTACTTCCTGCAGTGACTTTGAATAAATGAAGGAAAAAATTCAAAAAAAAAAAAAAAAANACNC
NGGGGGGGCCCCGGACCCAAATCCNCCNNAANGGGNGGCNTNNANAANNNACAAGGTGCGGCACCT
GGAGCACANAACAAGGTTCTGGANACCAAATGGACCTNCTGCACACANAAGACTGCCCGGACAACAT
AACAACATGGTTTGANAACTNCATTAAGAACCTTCNGTNGGNAGNTGGAAACTCTGGCCCANGAAAA
ACTTAAACTTGGAAAGTGGNNCTTGGCAACNNCCAGGGGCTGGGGGGAGGACTTCNAGACCCANGN
TTNNNGGNNNNAAATCCNAAANCCCCCANACNTNGGANAAATGAANTTTNNNNTTCNTNNAAAAAGG
GTNNNGGNTNAAACTTNNCNTNNACCAAGGNNNAACNTGGNNNCCCCCCCNNGNAANGGGNNTNNN
TNNNNAAAAAANAAANTTTTTNCAGGGNNANNTTTTNAAAAAAANNAAAANCCNNGNNNAANNN
CNNNNNTAAAAATTTTTNNACCCNNNCCGGGGGNCCNTNNNCNNTGGNNNAAAAAANCCNNNNNNC
NTTGGNNCNNGG
GCCTATCTGGAGCTNCACCNCNGNGGCGGCCGCTCTAGAACTAGTGGATCCCCCGGGCTGCAGGANTT
CGNNNCGNGGGTTCACTTTNCATAGTAACNNGGNCACACCATTAGGTTTCATGTATCACATTTAACCA
NGNCAGGNNACATNTNCCATGGGNCGNTCCAGCTTACTNGGATTACAGGCACNCTACCATTCATGGCA
TNAGTCCTGNNTACNANCAACCAGCTTNNNTGCATGCCNTGCAGCCATATNTTTGCNGCNTTATGAT
AACCAAAGCNTAGCCNTGTCAATGGGATGNCTTGAAATTGNNCANNCTGNTNCGGTGANTGTNTTTCG
TTTNNANTTTGANCTTGTTTACAGAATAACTCACCTGCATGGNCTCNTNACTANATCNACTGANAGAA
ANCTTGAGAAANATAACCTTTCTNCTGGGGGGGNCTCCCCNTGCCTCTAAATCCCTTTAATGGCNACC
GTCCCCCTCAGGCNCACCNTGGNGACCTGTGACANAGGATTTTTCAAAATTACNCCCCTCCTNTGTCCC
AGGCCCCANACTTTNTCCAAGGGACAACAGCAAACCAAAANTGCCATNCAAAGGTCCANCANCCTTTT
TGCCCCCATTTTCCCACTTTGCCATTGGGGNGCAAGTTTGCCTTTTGCCAAACCNCAAAGTNGGAGGGA
ANTNGGAAATGGGACCAACTTTTTACNATGGGANTAAAAACGGTTTTTTNTAACNAAANANAACCCCT
TTCCCAAAAGGGGGGGNTTTNTNGGGNGGGGGCCCTTTNTTTTTTTCACNGGNAAANANGGGANAGG
GTTTTTTTTTTTTNTNCCCCCCTTTTGGAGGCCTTTNTTTTTTTTNNNTTTTACCCCNTNCAAAANAATTN
CCCTTTCAAGGGGCCCAAACCTAAAGGGTAAAACAAACCTCCNGTTTTGGGNCCCCTTTGGAAAAAAN
CCNGGGGAAGGGCCCCCCCTNANGGGGTTTTTTTTTTTTTTCAGGGCTTTTTTTTGGAAAGGGGCCCCC
AGGGAAT
ANCAANNCTGGAGCTCCCCGCGGTGGCGGCCGCTCTAGAACTAGTGGATCCCCCGGGCTGCAGGAATT
CGGCACGAGGCTGAGAAGCCANATNCTAAGGAGAAAAAACCTGAAGCCAAGAAGGCTGATGCTGGC
ANGAAGGCTAAAAAGGTGGANAAGGTGAAGAAAGTNNANAAGGGGAAGCCCCACTGCAGCCGANAT
CCTGTCCTGGNCAGAGGAATTGGCAGATATTCCCGATCAGCCATGTACTCCAGAAAGGCCTTGTACAA
GAGAAAGTNTTCAGCTGCTAAATCCAAGGTCNAAAAGAAAAAGAAGGTTCGNGTTCTTGCTACTGTCA
CAAAACCATTTGGTGGAGACAAGAATGGTGGTACCCGAGTGGTCAAACTTCGCAAAATGCCTAGGTAT
TACCCTACTGAAGATGTGCCTCGAAAACTGTTGAGTCATGGCAAAAAACCCTTCAGTAAGCATGTGAG
GAAGCTGCGTGCCAGCATCACTCCTGGGACCATCCTGATCATCCTCACTGGGCCGCCACAAAAGGCAA
GAAGGTTGNTTTCCTGAAGCAGCTTGGGCAGTGGGCTTTGCTACTTTGTGAACTTGGGCCTTTTCCC
TCAATCGAAGTTTCTTTTTGNGTANAACACACCCAAAAANTTTGTCATTTGNCNNCCTCCCNCCAAAA
ATCGATATNCAGNGGGNGTGAAAATNCCCAAAACATTTTCACTTGACNCCTTACNTTTAANAAAAAA
AAAAAGCTTTGGTTNAANCCCCANACCCCCCGGGNGGGGCGANAANTCTTTCCNCCCCCGGNGGNGG
NGGGGNAAATTNCCGAGAATNCCCCNNNNCCCNCGCCAANGGNTCCAATANAAAAAAAGCTTTTNGG
ACCTTCCCNAAAATTTTTTGGGNAAAAAAAANAAAAAAAGGTTTGNCCCTTTAAAATTTCCAANGGGG
GTTAACCTTTCGGGTTNCCCNGNGNTTTGGN

We claim:

1. An expressed sequence tag (“EST”), wherein said EST is an isolated, enriched, or purified nucleic acid sequence representing all or part of a gene, the expression of which, or its complementary sequence, in a cell identifies said cell as a developmentally competent cell.

2. An expressed sequence tag (“EST”), wherein said EST is an isolated, enriched, or purified nucleic acid sequence representing all or part of a gene, the expression of which, or its complementary sequence, in a cell identifies said cell as a developmentally incompetent cell.

3. A gene expression database comprising:
two or more expressed sequence tags (“ESTs”), wherein said ESTs are isolated, enriched, or purified nucleic acid sequences representing all or part of two or more genes, the expression of which, or their complementary sequences, in a cell identifies said cell as a developmentally competent cell.

4. A gene expression database comprising:
two or more expressed sequence tags (“ESTs”), wherein said ESTs are isolated, enriched, or purified nucleic acid sequences representing all or part of two or more genes, the expression of which, or their complementary sequences, in a cell identifies said cell as a developmentally incompetent cell.

5. A gene expression database comprising:
one or more first expressed sequence tags (“ESTs”), wherein said ESTs are isolated, enriched, or purified nucleic acid sequences representing all or part of one or more genes, the expression of which, or their complementary sequences, in a cell identifies said cell as a developmentally competent cell; and
one or more second expressed sequence tags, wherein said ESTs are isolated, enriched, or purified nucleic acid sequences representing all or part of one or more genes, the expression of which, or their complementary sequences, in a cell identifies said cell as a developmentally incompetent cell.

6. A gene expression database according to any one of claims 3, 4, or 5, comprising at least 10 different ESTs.

7. A gene expression database according to any one of claims 3, 4, or 5, comprising at least 100 different ESTs.

8. A gene expression database according to any one of claims 3, 4, or 5, comprising at least 1000 different ESTs.

9. A gene expression database according to any one of claims 3, 4, or 5, comprising at least 5000 different ESTs.

10. A gene expression database according to any one of claims 3, 4, or 5, comprising at least 10,000 different ESTs.

11. A gene expression database according to any one of claims 3, 4, or 5, wherein ESTs are obtained from the same species as said cell.

12. A gene expression database according to any one of claims 3, 4, or 5, wherein said cell is a mammalian cell.

13. A gene expression database according to claim 12, wherein said mammalian cell is an ungulate cell.

14. A gene expression database according to claim 13, wherein said ungulate cell is a bovine cell.

15. A method of identifying an expressed sequence tag (“EST”), wherein said EST is an isolated, enriched, or purified nucleic acid sequence representing all or part of a gene, the expression of which, or its complementary sequence, identifies said cell as a developmentally competent cell, the method comprising:
comparing one or more first nucleic acid molecules obtained from one or more embryos produced by nuclear transfer using a developmentally competent nuclear donor cell to one or more second nucleic acid molecules obtained from one or more embryos produced by nuclear transfer using a developmentally incompetent nuclear donor cell; and
identifying a nucleic acid molecule present in said first nucleic acid molecules that is not present at a detectable level in said second nucleic acid molecules.

16. A method of identifying an expressed sequence tag (“EST”), wherein said EST is an isolated, enriched, or purified nucleic acid sequence representing all or part of a gene, the expression of which, or its complementary sequence, identifies said cell as a developmentally incompetent cell, the method comprising:
comparing one or more first nucleic acid molecules obtained from one or more embryos produced by nuclear transfer using a developmentally competent nuclear donor cell to one or more second nucleic acid molecules obtained from one or more embryos produced by nuclear transfer using a developmentally incompetent nuclear donor cell; and
identifying a nucleic acid molecule present in said second nucleic acid molecules that is not present at a detectable level in said first nucleic acid molecules.

17. A method of preparing a gene expression database comprising two or more expressed sequence tags (“ESTs”), wherein said ESTs are isolated, enriched, or purified nucleic acid sequences representing all or part of two or more genes, the expression of which, or their complementary sequences, identifies said cell as a developmentally competent cell, the method comprising:
comparing a first plurality of nucleic acid molecules obtained from one or more embryos produced by nuclear transfer using a developmentally competent nuclear donor cell to a second plurality of nucleic acid molecules obtained from one or more embryos produced by nuclear transfer using a developmentally incompetent nuclear donor cell;
identifying two or more nucleic acid molecules present in said first plurality of nucleic acid molecules that are not present at a detectable level in said second plurality of nucleic acid molecules to provide two or more identified nucleic acid molecules; and
combining said identified nucleic acid molecules in a gene expression database.

18. A method of preparing a gene expression database comprising two or more expressed sequence tags (“ESTs”), wherein said ESTs are isolated, enriched, or purified nucleic acid sequences representing all or part of two or more genes, the expression of which, or their complementary sequences, identifies said cell as a developmentally incompetent cell, the method comprising:
comparing a first plurality of nucleic acid molecules obtained from one or more embryos produced by nuclear transfer using a developmentally competent nuclear donor cell to a second plurality of nucleic acid molecules obtained from one or more embryos produced by nuclear transfer using a developmentally incompetent nuclear donor cell;
identifying two or more nucleic acid molecules present in said second plurality of nucleic acid molecules that are not present at a detectable level in said first plurality of nucleic acid molecules to provide two or more identified nucleic acid molecules; and
combining said identified nucleic acid molecules in a gene expression database.

19. A method of preparing a gene expression database comprising one or more first expressed sequence tags (“ESTs”), wherein said EST is an isolated, enriched, or purified nucleic acid sequence representing all or part of a gene, the expression of which, or its complementary sequence, identifies said cell as a developmentally competent cell, and one or more second expressed sequence tags, the expression of which in a cell identifies said cell as a developmentally incompetent cell, the method comprising:
comparing a first plurality of nucleic acid molecules obtained from one or more embryos produced by nuclear transfer using a developmentally competent nuclear donor cell to a second plurality of nucleic acid molecules obtained from one or more embryos produced by nuclear transfer using a developmentally incompetent nuclear donor cell;
identifying one or more nucleic acid molecules present in said first plurality of nucleic acid molecules that are not present at a detectable level in said second plurality of nucleic acid molecules and identifying one or more nucleic acid molecules present in said second plurality of nucleic acid molecules that are not present at a detectable level in said first plurality of nucleic acid molecules to provide two or more identified nucleic acid molecules; and
combining said identified nucleic acid molecules in a gene expression database.

20. A method according to any one of claims 17, 18, or 19, wherein said comparing step comprises comparing said first and/or said second plurality of nucleic acid molecules to a reference nucleic acid library obtained from an animal of the same species as said developmentally competent or said developmentally incompetent nuclear donor cell.

21. A method of producing one or more animals by nuclear transfer procedures using a competent nuclear donor cell, the method comprising:
(a) performing one or more nuclear transfer procedures to provide one or more nuclear transfer embryos;
(b) culturing each of said nuclear transfer embryos to provide one or more embryos comprising at least two cells;
(c) separating at least one cell from each of said embryos to provide one or more isolated embryonic cell populations;
(d) determining the developmental competence of each of said embryonic cell populations by comparing one or more nucleic acid molecules obtained from each of said embryonic cell populations to a gene expression database, whereby said comparison identifies those embryos resulting from nuclear transfer of a developmentally competent nuclear donor cell; and
(e) implanting embryos resulting from nuclear transfer of a developmentally competent nuclear donor cell into one or more recipient females for development into said one or more animals.

22. A method according to claim 21, wherein said one or more animals are one or more mammalian animals.

23. A method according to claim 22, wherein said one or more mammalian animals are one or more bovine animals.

24. A method according to claim 21, wherein said nuclear transfer procedures comprise using a transgenic nuclear donor cell.

25. A method of identifying a developmentally competent nuclear donor cell line, the method comprising:
(a) separating one or more cells from a cell line to provide one or more separated cells;
(b) performing one or more nuclear transfer procedures using each of said separated cells to provide one or more nuclear transfer embryos; and
(c) determining the developmental competence of each of said nuclear transfer embryos by comparing a plurality of nucleic acid molecules obtained from each of said embryos to a gene expression database, whereby said comparison identifies those embryos resulting from nuclear transfer of a developmentally competent nuclear donor cell.

26. A method according to claim 25, wherein said method further comprises culturing said nuclear transfer embryos prior to determining the developmental competence of each of said nuclear transfer embryos.

27. A method according to claim 25, wherein said one or more animals are one or more mammalian animals.

28. A method according to claim 27, wherein said one or more mammalian animals are one or more bovine animals.

29. A method according to claim 25, wherein said nuclear donor cell line is a transgenic nuclear donor cell line.

30. A method of producing one or more embryos by nuclear transfer procedures using a competent nuclear donor cell, the method comprising:
(a) performing one or more nuclear transfer procedures to provide one or more nuclear transfer embryos;
(b) culturing each of said nuclear transfer embryos to provide one or more embryos comprising at least two cells;
(c) separating at least one cell from each of said embryos to provide one or more isolated embryonic cell populations; and
(d) determining the developmental competence of each of said embryonic cell populations by comparing one or more nucleic acid molecules obtained from each of said embryonic cell populations to a gene expression database, whereby said comparison identifies those embryos resulting from nuclear transfer of a developmentally competent nuclear donor cell.

31. A method to assess the effect of one or more changes in a nuclear transfer protocol, the method comprising:
(a) performing one or more nuclear transfer procedures according to a first nuclear transfer protocol to produce one or more first protocol nuclear transfer embryos;
(b) performing one or more nuclear transfer procedures according to a second nuclear transfer protocol comprising one or more changes to said first nuclear transfer protocol, to produce one or more second protocol nuclear transfer embryos;
(c) determining the developmental competence of each of said first protocol and second protocol nuclear transfer embryos by comparing a plurality of nucleic acid molecules obtained from each of said embryos to a gene expression database, whereby said comparison identifies those embryos; and
(d) assessing the effect of said one or more changes by comparing the developmental competence of said first protocol nuclear transfer embryos to the developmental competence of said second protocol nuclear transfer embryos.

32. A method according to claim 31, wherein said method further comprises culturing said first protocol nuclear transfer embryos and/or said second protocol nuclear transfer embryos prior to determining the developmental competence of each of said nuclear transfer embryos.

33. A method according to claim 31, wherein said nuclear transfer procedures comprise using a mammalian nuclear donor cells.

34. A method according to claim 33, wherein said mammalian nuclear donor cells are bovine nuclear donor cells.

35. A nucleic acid array comprising:
two or more different nucleic acid molecules, the expression of which in a cell identifies said cell as a developmentally competent cell, wherein each of said different nucleic acid molecules is affixed to a solid matrix.

36. A nucleic acid array comprising:
two or more different nucleic acid molecules, the expression of which in a cell identifies said cell as a developmentally incompetent cell, wherein each of said different nucleic acid molecules is affixed to a solid matrix.

37. A nucleic acid array comprising:
at least one nucleic acid molecule, the expression of which in a cell identifies said cell as a developmentally competent cell; and
at least one nucleic acid molecule, the expression of which in a cell identifies said cell as a developmentally incompetent cell,
wherein each of said different nucleic acid molecules is affixed to a solid matrix.

38. A nucleic acid array according to any one of claims 35, 36, or 37, wherein each different nucleic acid molecule is positioned at a different spatial location on said solid matrix.

39. A nucleic acid array according to any one of claims 35, 36, or 37, wherein said solid matrix comprises a material selected from the group consisting of polyacrylamide gel, agarose gel, nitrocellulose membrane, nylon membrane, glass, magnetic materials, magnetic beads, polymeric beads, or silicon.

40. A gene expression database comprising:
two or more expressed sequence tags (“ESTs”), wherein said ESTs are isolated, enriched, or purified nucleic acid sequences representing all or part of two or more genes, the expression of which, or their complementary sequences, in a stem cell identifies said stem cell as capable of committing to a specific cell lineage.

41. A gene expression database comprising:
two or more expressed sequence tags (“ESTs”), wherein said ESTs are isolated, enriched, or purified nucleic acid sequences representing all or part of two or more genes, the expression of which, or their complementary sequences, in a stem cell identifies said stem cell as incapable of committing to a specific cell lineage.

42. A gene expression database comprising:
at least one expressed sequence tag (“ESTs”), wherein said EST is an isolated, enriched, or purified nucleic acid sequence representing all or part of a gene, the expression of which, or its complementary sequence, in a stem cell identifies said stem cell as capable of committing to a specific cell lineage; and
at least one expressed sequence tag, wherein said EST is an isolated, enriched, or purified nucleic acid sequence representing all or part of a gene, the expression of which, or its complementary sequence, in a stem cell identifies said stem cell as incapable of committing to a specific cell lineage.

43. A method of preparing a gene expression database comprising two or more expressed sequence tags (“ESTs”), wherein said ESTs are isolated, enriched, or purified nucleic acid sequences representing all or part of two or more genes, the expression of which, or their complementary sequences, in a stem cell identifies said stem cell as capable of committing to a specific cell lineage, the method comprising:
comparing a first plurality of nucleic acid molecules obtained from one or more stem cells known to be capable of differentiation into a specific cell type to a second plurality of nucleic acid molecules obtained from one or more stem cells known to be incapable of differentiation into a specific cell type; and
identifying two or more nucleic acid molecules present in said first plurality of nucleic acid molecules that are not present at a detectable level in said second plurality of nucleic acid molecules.

44. A method of preparing a gene expression database comprising two or more expressed sequence tags (“ESTs”), wherein said ESTs are isolated, enriched, or purified nucleic acid sequences representing all or part of two or more genes, the expression of which, or their complementary sequences, in a stem cell identifies said stem cell as incapable of committing to a specific cell lineage, the method comprising:
comparing a first plurality of nucleic acid molecules obtained from one or more stem cells known to be capable of differentiation into a specific cell type to a second plurality of nucleic acid molecules obtained from one or more stem cells known to be incapable of differentiation into a specific cell type; and
identifying two or more nucleic acid molecules present in said second plurality of nucleic acid molecules that are not present at a detectable level in said first plurality of nucleic acid molecules.

45. A method of preparing a gene expression database comprising one or more expressed sequence tags (“ESTs”), wherein said ESTs are isolated, enriched, or purified nucleic acid sequences representing all or part of one or more genes, the expression of which, or their complementary sequences, in a stem cell identifies said stem cell as capable of committing to a specific cell lineage, and one or more expressed sequence tags, wherein said ESTs are isolated, enriched, or purified nucleic acid sequences representing all or part of one or more genes, the expression of which, or their complementary sequences, in a stem cell identifies said stem cell as incapable of committing to a specific cell lineage the method comprising:
comparing a first plurality of nucleic acid molecules obtained from one or more stem cells known to be capable of differentiation into a specific cell type to a second plurality of nucleic acid molecules obtained from one or more stem cells known to be incapable of differentiation into a specific cell type; and
identifying one or more nucleic acid molecules present in said first plurality of nucleic acid molecules that are not present at a detectable level in said second plurality of nucleic acid molecules, and identifying one or more nucleic acid molecules present in said second plurality of nucleic acid molecules that are not present at a detectable level in said first plurality of nucleic acid molecules.

46. A method of identifying one or more molecules that induce developmental competence in a cell line, the method comprising:
(a) contacting a developmentally incompetent cell line with one or more molecules to provide a treated cell line;
(b) separating one or more cells from said treated cell line to provide one or more separated cells;
(c) performing one or more nuclear transfer procedures using each of said separated cells to provide one or more nuclear transfer embryos; and
(d) determining the developmental competence of each of said nuclear transfer embryos by comparing a plurality of nucleic acid molecules obtained from each of said embryos to a gene expression database, whereby said comparison identifies those embryos resulting from nuclear transfer of a developmentally competent nuclear donor cell.

47. A method according to claim 46, wherein said cell line is selected from the group consisting of a cell line derived from cells isolated from an embryo arising from the union of two gametes in vitro or in vivo; an embryonic stem cell line; a cell line arising from inner cell mass cells isolated from of embryos; a cell line obtained from pre-blastocyst cells; a fetal cell line; a primordial germ cell line; a germ cell line, an embryonic germ cell line, a somatic cell line isolated from an animal; a cumulus cell line; an amniotic cell line; a fetal fibroblast cell line; a genital ridge cell line; a differentiated cell line; a lineage-specific cell line; and a totipotent cell line.

48. A method of inducing totipotence in a cell line, the method comprising:
(a) contacting said cell line with one or more molecules identified as inducing developmental incompetence in a cell line by the method of claim 46, whereby one or more cells of said cell line become developmentally competent.

49. A method of treating a disease in an animal by inducing totipotence in one or more cells of the animal, the method comprising:
(a) administering one or more molecules identified as inducing developmental competence in a cell line by the method of claim 46 to said animal, whereby one or more cells of said animal become developmentally competent.

50. A method according to claim 49, wherein said one or more cells are selected from the group consisting of neurons, glial cells, muscle cells, neural cells, and bone marrow cells.

51. A method of identifying one or more molecules that induce developmental incompetence in a cell line, the method comprising:
(a) contacting a developmentally competent cell line with one or more molecules to provide a treated cell line;
(b) separating one or more cells from said treated cell line to provide one or more separated cells;
(c) performing one or more nuclear transfer procedures using each of said separated cells to provide one or more nuclear transfer embryos; and
(d) determining the developmental competence of each of said nuclear transfer embryos by comparing a plurality of nucleic acid molecules obtained from each of said embryos to a gene expression database, whereby said comparison identifies those embryos resulting from nuclear transfer of a developmentally incompetent nuclear donor cell.

52. A method of preventing a full term pregnancy in an animal by inducing developmental incompetence in one or more cells in said animal, the method comprising:
(a) administering one or more molecules identified as inducing developmental incompetence in a cell line by the method of claim 51 to said animal, whereby one or more cells in said animal become developmentally incompetent.

53. A method according to claim 52, wherein said one or more cells are selected from the group consisting of spermatocytes, spermatozoa, oocytes, fertilized oocytes, and embryos.

54. A method of treating a disease in an animal by inhibiting totipotence in one or more cells of the animal, the method comprising:
(a) administering one or more molecules identified as inducing developmental incompetence in a cell line by the method of claim 51 to said animal, whereby one or more cells in said animal become developmentally incompetent.

55. A method of identifying one or more molecules that induce lineage-specific development in a stem cell line, the method comprising:
(a) contacting a stem cell line known to be incapable of differentiation into a specific cell type with one or more molecules to provide a treated cell line;
(b) determining the capability of said treated cell line to differentiate into said specific cell type by comparing a plurality of nucleic acid molecules obtained from said treated cell line to a gene expression database, whereby said comparison identifies stem cells capable of committing to a specific cell lineage.

56. A method of inducing development of a cell into a specific cell type, the method comprising:
(a) contacting a cell with one or more molecules identified as inducing lineage-specific development in a cell line by the method of claim 55, whereby said cell line develops into said specific cell type.

57. A method of treating a disease in an animal by inducing development of one or more cells of the animal into a specific cell type, the method comprising:
(a) administering one or more molecules identified as inducing developmental competence in a cell line by the method of claim 55 to said animal, whereby said one or more cells of the animal develop into said specific cell type.

58. A method of identifying one or more molecules that inhibit lineage-specific development in a stem cell line, the method comprising:
(a) contacting a stem cell line known to be capable of differentiation into a specific cell type with one or more molecules to provide a treated cell line;
(b) determining the capability of said treated cell line to differentiate into said specific cell type by comparing a plurality of nucleic acid molecules obtained from said treated cell line to a gene expression database, whereby said comparison identifies stem cells incapable of committing to a specific cell lineage.

59. A method of inhibiting development of a cell into a specific cell type, the method comprising:
(a) contacting a cell with one or more molecules identified as inhibiting lineage-specific development in a cell line by the method of claim 58, whereby said cell line is prevented from developing into said specific cell type.

60. A method of treating a disease in an animal by inducing development of one or more cells of the animal into a specific cell type, the method comprising:
(a) administering one or more molecules identified as inducing developmental incompetence in a cell line by the method of claim 59 to said animal, whereby said one or more cells of the animal are prevented from developing into said specific cell type.
